(19) United States
(12) Patent Application Publication
(10) Pub. No.: US 2023/0079045 A1
(43) Pub. Date: Mar. 16, 2023

(54) ANTI-INFLUENZA VIRUS COMPOUND, PREPARATION METHOD AND USE THEREOF

(71) Applicant: Sichuan Haisco Pharmaceutical Co., Ltd., Chengdu City, Sichuan Province (CN)

(72) Inventors: Yao Li, Chengdu City (CN); Lei Chen, Chengdu City (CN); Guobiao Zhang, Chengdu City (CN); Wenjing Wang, Chengdu City (CN); Zongjun Shi, Chengdu City (CN); Gang Hu, Chengdu City (CN); Haitao Huang, Chengdu City (CN); Haodong Wang, Chengdu City (CN); Bo Xu, Chengdu City (CN); Xiaobo Zhang, Chengdu City (CN); Guoliang LIU, Chengdu City (CN); Dengyu Zheng, Chengdu City (CN); Shilin Huang, Chengdu City (CN); Jianfei Zhao, Chengdu City (CN); Changwei Song, Chengdu City (CN); Chen Zhang, Chengdu City (CN); Fei Ye, Chengdu City (CN); Jia Ni, Chengdu City (CN); Pangke Yan, Chengdu City (CN)

(73) Assignee: Sichuan Haisco Pharmaceutical Co., Ltd., Chengdu City, Sichuan Province (CN)

(21) Appl. No.: 17/636,977

(22) PCT Filed: Aug. 24, 2020
(86) PCT No.: PCT/CN2020/110764

§ 371 Date: Feb. 21, 2022

(30) Foreign Application Priority Data
Aug. 22, 2019 (CN) 201910751699.8
Sep. 23, 2019 (CN) 201910898511.2
Oct. 15, 2019 (CN) 201910970947.8
Nov. 7, 2019 (CN) 201911065183.4
Nov. 18, 2019 (CN) 201911124201.1
Dec. 4, 2019 (CN) 201911216664.0
Dec. 25, 2019 (CN) 201911352521.2
Jan. 9, 2020 (CN) 202010020571.7
Feb. 28, 2020 (CN) 202010129782.4
Mar. 27, 2020 (CN) 202010214019.1
Apr. 27, 2020 (CN) 202010341066.2
May 21, 2020 (CN) 202010432952.6
Jun. 1, 2020 (CN) 202010460155.9
Aug. 3, 2020 (CN) 202010765603.6

(51) Int. Cl. C07D 413/14; C07D 451/02; C07D 487/08; C07D 471/10; C07D 487/10; C07D 471/08; C07D 401/14; C07D 417/14; C07D 405/14; A61P 31/16

(52) U.S. Cl. CPC C07D 413/14; C07D 451/02; C07D 487/08; C07D 471/10; C07D 487/10; C07D 471/08; C07D 401/14; C07D 417/14; C07D 405/14; A61P 31/16

(57) ABSTRACT

Provided are a compound of formula (I-a), an isomer thereof or a pharmaceutically acceptable salt thereof as a Hemagglutinin inhibitor, and a preparation method thereof. The compound is useful for preparing a medicament for treating a disease related to Hemagglutinin.

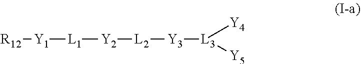

### TECHNICAL FIELD

The present disclosure falls within the field of medicinal chemistry. In particular, the present disclosure relates to an anti-influenza virus compound, an isomer thereof or a pharmaceutically acceptable salt thereof, a preparation method therefor and the use thereof as a Hemagglutinin (HA) inhibitor.

### BACKGROUND ART

Influenza (flu for short) is an acute respiratory infection caused by influenza viruses and is also a highly infectious and fast-transmitting disease. The most notable features of influenza in epidemiology comprise sudden outbreak and rapid dispersion, resulting in various degrees of epidemics. Influenza has a certain seasonality (the epidemic peak in northern China generally come in winter and spring, whereas influenza is epidemic throughout the year in south China with the peak generally in summer and winter). More and more studies have shown that the seasonality of influenza is highly diverse in tropical area, especially in Asia, with both a half-yearly or a year-round periodic epidemic, and a year-round cycle. According to statistics on the influenza attack rate of unvaccinated population in 32 randomized controlled cohorts of influenza vaccination worldwide, the attack rate of symptomatic influenza is 4.4% for adults and 7.2% for population over 65 years old, and the attack rate of all influenza (including asymptomatic influenza infection) in adults is 10.7%. According to the WHO reports, seasonal influenza epidemic can cause 3-5 million severe cases and 290,000-650,000 deaths globally each year. In northern and southern cities of China, the average annual excess mortalities caused by influenza-related respiratory and circulatory system diseases are 12.4 per 100,000 and 8.8 per 100,000, respectively. From a global perspective, influenza is one of the main reasons for hospitalization and excess death among population ≥65 years old. The influenza epidemic can also cause a large number of school-age children to absent from school and parents to absent from work, increase outpatients and hospitalization costs, and cause heavy social and economic burdens.

Influenza viruses are divided into four types: A, B, C and D according to nucleocapside protein (NP) and matrix protein (MP). RNA polymerases used in the replication of influenza viruses do not have corrective activity, making an error for about every 10,000 nucleotides they copy and resulting in a high frequency of mutation; moreover, due to the segmental influenza virus genome, gene reassortment may occur when viruses of different subtypes or different genotypes infect the same cell at the same time, resulting in a relatively higher number of mutations in the viral genome. Influenza A virus often mutates due to large number of hosts and structural characteristics thereof; and under the immune pressure of population, a significant antigenic variant arises every 2-3 years, which makes general susceptibility of the population and causes seasonal epidemics. For example, the 2009 pandemic influenza A (H1N1) virus is a reassortant strain derived from poultry, pig and human.

Influenza vaccination is an effective way to prevent influenza, which can significantly reduce the risk of influenza and serious complications. However, seasonal influenza vaccine still has limitations. Firstly, influenza vaccine has a good protective effect on healthy adults but has a poor effect on the elderly. Secondly, seasonal influenza vaccine must be updated every year to prevent the type of viruses which are predicted to be pandemic in a new influenza season. However, due to the emergence of a new virus type, the protective effect of the vaccine may not be as expected.

In addition to influenza vaccination, treatment with anti-influenza virus medicaments is the most fundamental and important part of influenza treatment, but influenza viruses are prone to mutation, which causes resistance to antiviral medicaments. The medicaments currently on the market for the treatment of influenza viruses can be divided into the following two types according to mechanisms of action: a neuraminidase inhibitor and an M2 ion channel blocker. The mechanism of action of a neuraminidase inhibitor is to prevent viruses from being released from infected cells and invading neighboring cells, thus reducing replication of the viruses in the body, and a neuraminidase inhibitor is active against both influenza A and B. Neuraminidase inhibitors marketed in China include oral oseltamivir, inhaled zanamivir and intravenous peramivir. It has been reported that more than 80% of seasonal influenza A viruses (H1N1) are resistant to oseltamivir. Zanamivir for inhalation is not recommended for severe patients or patients suffering from complications. The current clinical application data of peramivir is limited, and adverse reaction thereof should be closely observed. M2 ion channel blockers block the ion channel of influenza virus M2 protein, thereby inhibiting virus replication, but the blockers only have an inhibitory effect on influenza A viruses. M2 ion channel blockers comprise amantadine and rimantadine. Amantadine and rimantadine are only effective against influenza A viruses. However, the current monitoring data show that influenza A viruses are resistant to amantadine and rimantadine and are not recommended by the Bureau of Medical Administration, National Health Commission of the People's Republic of China.

According to the surface antigen hemagglutinin (HA) and neuraminidase (NA) protein structures and gene characteristics, influenza A viruses are divided into different subtypes. Hemagglutinins that have been discovered currently comprise 18 subtypes (H1 to H18), and neuraminidases that have been discovered currently comprise 10 subtypes (N1 to N10). The entry of a virus into a host cell is the first important step of the initiation of a viral replication cycle. Influenza virus protein hemagglutinins can recognize the potential binding site of sialic acid (SA) (N-acetylneuraminic acid) on glycoproteins of a host cell. The HAs contained in influenza viruses which infect humans have high specificity to α2-6SA. After HAs bind to receptors, the viruses are endocytosed; and the acidic pH of endosomes causes changes on the conformation of HA proteins, thereby regulating the internal fusion of the viruses and recipient cells and releasing RNPs of the viruses into cytoplasm. Therefore, by using HAs as targets and binding to the HAs, the conformational change of HA2 caused by low pH conditions is inhibited, thereby inhibiting the process of fusion of viral envelopes with host endosomal membranes, which has become a new anti-influenza virus strategy. Currently, there are multiple vaccines for HAs in the clinical stage, such as CR9114 (WO 2013/007770) and CR6261 (WO 2008/028946). Small molecule compounds with different structural features for the treatment of influenza have also been reported in the literature. A series of benzisoxazole compounds for the treatment of influenza are reported in WO 2012/144752. WO 2012/037119 reports that sulfonamide derivatives act on envelope glycoproteins of HAs and have an anti-influenza virus effect. In 2010, Roche reported a series of phenylacetamide derivatives, which showed high anti-influenza virus activity when used in combination with HA proteins (Bioorganic & Medicinal Chemistry Letters. 20(2010): 3507-3510). WO 2013/074965 reports that aminoalkyl phenol ether is used as an HA inhibitor for the treatment of influenza.

WO 2018/141854 reports a series of piperazine derivatives which are used to prevent and treat influenza by binding to conserved regions of HAs. WO 2011/147199 discloses a high-throughput and virtual screening method which can identify potential antiviral agents. The method is used to identify compounds which inhibit viral infections by binding to viral nucleoproteins. However, until now, there is no HA small molecule inhibitor entered into the clinical stage. Oral small molecules are superior to antibodies in the aspect of development costs and administration methods, can be used in both prevention and treatment, and are superior to vaccines and antibodies in terms of target population. Therefore, it is very necessary to develop more effective and safer small molecule medicaments targeting HAs for the treatment of influenza.

### SUMMARY OF THE DISCLOSURE

In view of the limitation of influenza vaccines currently on the market and the drug resistance to anti-viral medicaments, it is urgently required to develop a new anti-influenza virus compound for the treatment of influenza.

An object of the present disclosure is to provide an anti-influenza virus compound having a novel structure, an isomer thereof, or a pharmaceutically acceptable salt.

The present disclosure provides a compound represented by general formula (I), an isomer thereof or a pharmaceutically acceptable salt thereof:

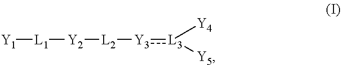

wherein: [symbol]
represents a single bond or a double bond;
Y₁, Y₂, Y₃, Y₄ and Y₅ are each independently selected from: C₃₋₁₂ carbocyclyl, C₅₋₁₅ aryl, 5-15 membered heteroaryl, 3-12 membered heterocyclyl, C₂₋₈ alkenyl, C₂₋₈ alkynyl,

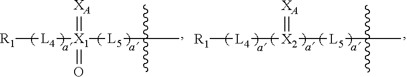

wherein
the above-mentioned groups are optionally substituted with 0-5 substituents selected from: halogen, ═O, hydroxyl, cyano, nitro, silyl, C₁₋₈ alkyl, C₁₋₈ alkoxy, C₂₋₈ alkenyl, C₂₋₈ alkynyl, C₃₋₈ cycloalkyl,

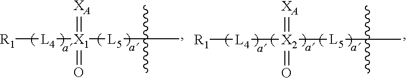

3-8 membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; X₁ is selected from P or S; X₂ is selected from C, PR₁ₐ or S; L₄ and L₅ are each independently selected from: a bond, NR₂, CR₃R₄, O, or S; X_{A} is selected from C, NR₂ₐ, O and S; a is 0 or 1; a′ is selected from 0, 1, 2, 3, 4, 5 or 6; R₁, R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the above-mentioned substituents are optionally further substituted with 0-5 groups selected from: halogen, C₁₋₈ alkyl, C₁₋₈ alkoxy or C₁₋₈ haloalkyl; or R₁ is absent;
or Y₂ is absent;

L₁ is absent or selected from: a bond, C₂₋₈ alkynyl, C₂₋₈ alkenyl,

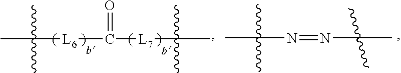

3-8 membered heterocyclyl, C₃₋₈ cycloalkyl, C₅₋₁₀ aryl or 5-10 membered heteroaryl, wherein the alkynyl, alkenyl,

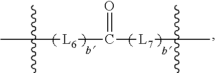

heterocyclyl, cycloalkyl, aryl or heteroaryl is optionally substituted with 0-5 groups selected from: hydroxyl, halogen, C₁₋₈ alkyl or C₁₋₈ haloalkyl;
L₂ is absent or selected from a bond,

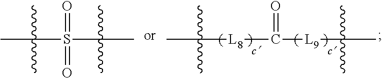

L₆, L₇, L₈ and L₉ are each independently selected from a bond, O, S, NR₅ or CR₆R₇; b and c are each independently selected from 0 or 1; b′ and c′ are each independently selected from 0, 1, 2, 3, 4, 5 or 6; R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl;
L₃ is absent or selected from: a bond,

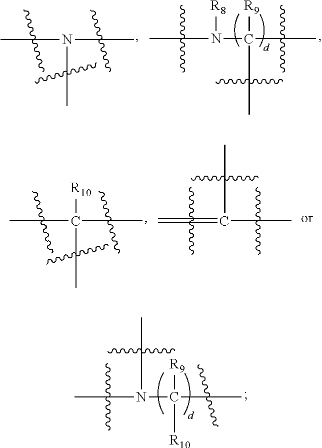

d is an integer selected from 1-6; and R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl.

According to some embodiments of the present disclosure, Y₁, Y₂ and Y₃ are each independently selected from 5-15 membered heteroaryl, C₅₋₁₅ aryl, 3-12 membered heterocyclyl or C₃₋₁₂ carbocyclyl.

According to some embodiments of the present disclosure, Y₄ is selected from C₅₋₁₅ aryl, 5-15 membered heteroaryl, 3-12 membered heterocyclyl, C₃₋₁₂ carbocyclyl, C₂₋₈ alkenyl or C₂₋₈ alkynyl.

According to some embodiments of the present disclosure, Y₅ is selected from 3-12 membered heterocyclyl, C₃₋₁₂ carbocyclyl, 5-15 membered heteroaryl, C₅₋₁₅ aryl,

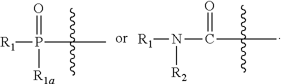

According to some embodiments of the present disclosure, Y₁ is selected from 5-10 membered heteroaryl, C₅₋₁₀ aryl or 3-8 membered heterocyclyl.

According to some embodiments of the present disclosure, Y₂ is selected from C₅₋₁₀ aryl or 5-10 membered heteroaryl.

According to some embodiments of the present disclosure, Y₃ is selected from C₃₋₁₂ carbocyclyl, 3-8 membered heterocyclyl, C₅₋₁₀ aryl or 5-10 membered heteroaryl.

According to some embodiments of the present disclosure, Y₄ is selected from C₅₋₁₀ aryl, 5-10 membered heteroaryl, C₂₋₈ alkenyl or C₂₋₈ alkynyl.

According to some embodiments of the present disclosure, Y₅ is selected from 3-8 membered heterocyclyl, 5-10 membered heteroaryl,

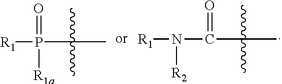

The present disclosure provides a compound represented by formula (II), an isomer thereof or a pharmaceutically acceptable salt thereof:

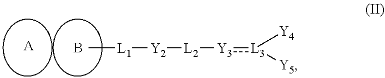

wherein
ring A is 3-8 membered heterocyclyl, 5-8 membered heteroaryl or C₅₋₁₀ aryl;
ring B is C₅₋₁₀ aryl, 5-8 membered heteroaryl, 3-8 membered heterocyclyl or C₃₋₈ carbocyclyl.

According to some embodiments of the present disclosure,

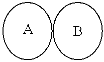

is selected from:

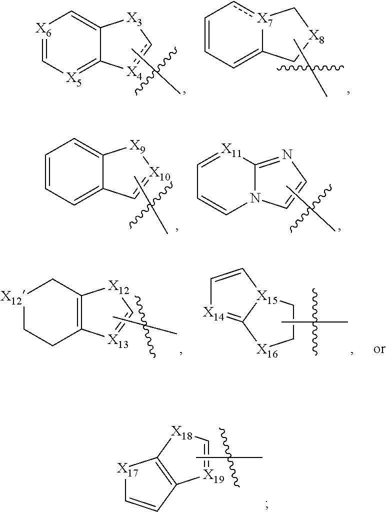

[symbol]

represents a single bond or a double bond;
X₃, X₄, X₅, X₆, X₇, X₉, X₁₂, X₁₂′, X₁₆, X₁₇ and X₁₈ are each independently selected from C, NR₁₁, O or S;
X₈, X₁₀, X₁₁, X₁₃, X₁₄, X₁₅ and X₁₉ are each independently selected from C or N;
and R₁₁ is selected from halogen, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, ═O, 3-8 membered heterocyclyl, C₅₋₁₀ aryl or 5-10 membered heteroaryl.

The present disclosure provides a compound represented by formula (III), an isomer thereof or a pharmaceutically acceptable salt thereof:

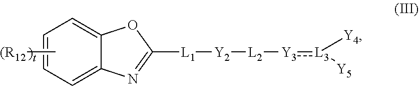

wherein
R₁₂ is selected from halogen, hydroxyl, cyano, nitro, C₁₋₈ alkyl, C₂₋₈ alkenyl, C₂₋₈ alkynyl, C₃₋₈ cycloalkyl,

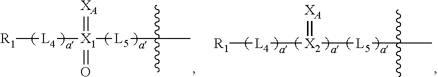

3-8 membered heterocyclyl, C₅₋₁₀ aryl or 5-10 membered heteroaryl.

According to some embodiments of the present disclosure, R₁₂ is optionally substituted with 0-5 groups selected from: halogen, ═O, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, ═O, 3-8 membered heterocyclyl, C₅₋₁₀ aryl or 5-10 membered heteroaryl, wherein the above-mentioned groups are optionally further substituted with one or more substituents selected from: halogen, ═O, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, ═O, 3-8 membered heterocyclyl, C₅₋₁₀ aryl or 5-10 membered heteroaryl.

According to some embodiments of the present disclosure, R₁₂ is optionally substituted with 0-5 groups selected from:

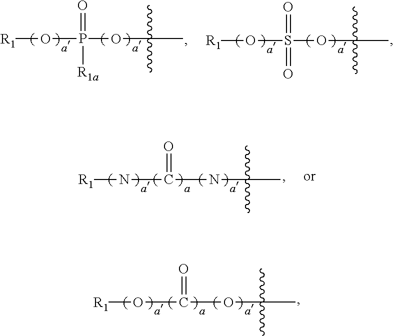

wherein the above-mentioned groups are optionally further substituted with one or more substituents selected from: halogen, ═O, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, ═O, 3-8 membered heterocyclyl, C₅₋₁₀ aryl or 5-10 membered heteroaryl.

According to some embodiments of the present disclosure, t is selected from 0, 1, 2, 3 or 4.

According to some embodiments of the present disclosure, R₁₂ is selected from: acetamido, C₃₋₈ cycloalkylacylamino, C₁₋₈ alkylacylamino, 3-8 membered heterocyclyl, carboxamido, C₁₋₈ alkylaminoacyl, C₃₋₈ cycloalkyloxyacylamino, C₃₋₈ cycloalkylamino, CN, C₁₋₈ haloalkylacylamino, 3-8 membered heterocyclylacylamino, C₃₋₈ cycloalkylacylamino, C₁₋₈ alkoxyacylamino, C₁₋₈ alkylaminoacylamino, 5-10 membered heteroaryl, C₁₋₈ alkoxy, 3-8 membered heterocyclyloxy, C₃₋₈ cycloalkylsulfonamido and C₁₋₈ alkylaminoacyloxy, wherein the above-mentioned groups are optionally further substituted with 0-5 substituents selected from: halogen, ═O, C₁₋₈ alkoxy, C₁₋₈ alkylcarbonyl, OH or C₃₋₈ cycloalkyl.

According to some embodiments of the present disclosure, Y₁ is selected from:

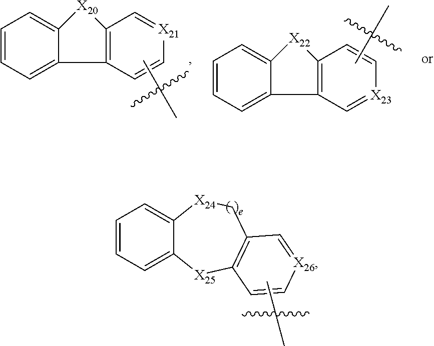

and X₂₀, X₂₂, X₂₄ and X₂₅ are each independently selected from a bond, C, O, NR₂ or S; e is 0 or 1; and X₂₁, X₂₃ and X₂₆ are selected from C or NR₂.

According to some embodiments of the present disclosure, Y₂ is selected from: C₅₋₁₀ aryl or 5-10 membered heteroaryl, wherein the aryl or heteroaryl is optionally substituted with 0-5 substituents selected from: cyano, hydroxyl, C₁₋₈ haloalkyl, C₁₋₈ alkoxy, C₁₋₈ alkyl or C₃₋₈ cycloalkyl.

According to some embodiments of the present disclosure, Y₂ is selected from: C₅₋₁₀ aryl or 5-10 membered heteroaryl, wherein the aryl or heteroaryl is optionally substituted with 0-5 substituents selected from: halogen, cyano, hydroxyl, C₁₋₈ haloalkyl, C₁₋₈ alkoxy, C₁₋₈ alkyl or C₃₋₈ cycloalkyl.

According to some embodiments of the present disclosure, Y₂ is selected from

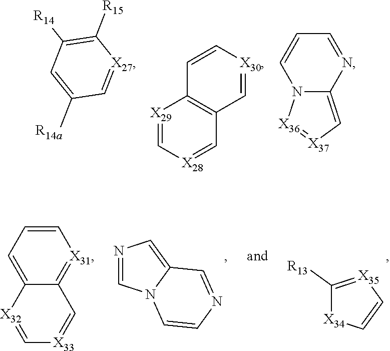

and X₂₇, X₂₈, X₂₉, X₃₀, X₃₁, X₃₂, X₃₃, X₃₅, X₃₆ and X₃₇ are selected from CR₁₆ or N; R₁₃, R₁₄, R₁₄ₐ and R₁₅ are selected from halogen, hydroxyl, cyano, nitro, C₁₋₈ alkyl, C₂₋₈ alkenyl, C₂₋₈ alkynyl, C₃₋₈ cycloalkyl,

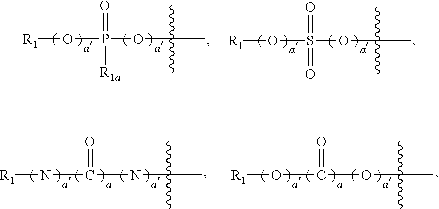

3-8 membered heterocyclyl, C₅₋₁₀ aryl or 5-10 membered heteroaryl; the above-mentioned groups are optionally further substituted with one or more substituents selected from: halogen, ═O, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, ═O, 3-8 membered heterocyclyl, C₅₋₁₀ aryl or 5-10 membered heteroaryl; X₃₄ is selected from CR₁₇, N, O or S; R₁₆ and R₁₇ are each independently selected from: H, cyano, C₁₋₈ alkyl, C₁₋₈ alkyl or C₃₋₈ cycloalkyl;
and according to some embodiments of the present disclosure, R₁₄ and R₁₅ together with the atoms to which they are attached form C₃₋₈ carbocyclyl, 3-8 membered heterocyclyl or 5-10 membered heteroaryl.

According to some embodiments of the present disclosure, Y₂ is selected from

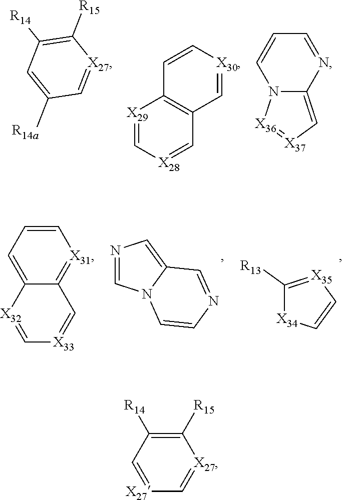

and
X₂₇, X₂₇′, X₂₈, X₂₉, X₃₀, X₃₁, X₃₂, X₃₃, X₃₅, X₃₆ and X₃₇ are selected from CR₁₆ or N;
R₁₃, R₁₄, R₁₄ₐ and R₁₅ are selected from halogen, hydroxyl, cyano, nitro, C₁₋₈ alkyl, C₂₋₈ alkenyl, C₂₋₈ alkynyl, C₃₋₈ cycloalkyl,

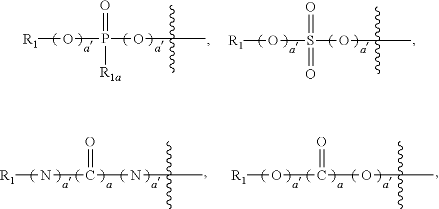

3-8 membered heterocyclyl, C₅₋₁₀ aryl or 5-10 membered heteroaryl; the above-mentioned groups are optionally further substituted with one or more substituents selected from: halogen, ═O, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, ═O, 3-8 membered heterocyclyl, C₅₋₁₀ aryl or 5-10 membered heteroaryl; X₃₄ is selected from CR₁₇, N, O or S; R₁₆ and R₁₇ are each independently selected from: H, cyano, C₁₋₈ alkyl, C₁₋₈ alkyl or C₃₋₈ cycloalkyl;
and according to some embodiments of the present disclosure, R₁₄ and R₁₅ together with the atoms to which they are attached form C₃₋₈ carbocyclyl, 3-8 membered heterocyclyl or 5-10 membered heteroaryl.

The present disclosure provides a compound represented by formula (IV), an isomer thereof or a pharmaceutically acceptable salt thereof:

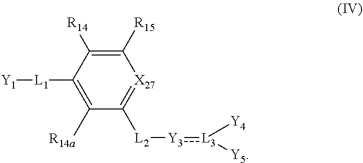

The present disclosure provides a compound represented by formula (V), an isomer thereof or a pharmaceutically acceptable salt thereof:

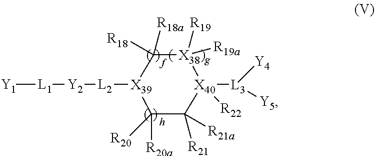

wherein X₃₈, X₃₉ and X₄₀ are each independently selected from: CR₂₃R₂₃ₐ, NR₂₄, O or S;
f, g and h are each independently selected from: 0, 1 or 2;
and R₁₈, R₁₈ₐ, R₁₉, R₁₉ₐ, R₂₀, R₂₀ₐ, R₂₁, R₂₁ₐ, R₂₂, R₂₃, R₂₃ₐ and R₂₄ are each independently selected from H, halogen, hydroxyl, cyano, nitro, C₁₋₈ alkyl, C₂₋₈ alkenyl, C₂₋₈ alkynyl, C₃₋₈ cycloalkyl,

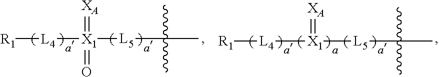

3-8 membered heterocyclyl, C₅₋₁₀ aryl or 5-10 membered heteroaryl.

According to some embodiments of the present disclosure, R₁₉ or R₁₉ₐ is absent.

According to some embodiments of the present disclosure, R₂₂ is absent.

According to some embodiments of the present disclosure, R₁₈/R₁₈ₐ together with the atoms to which they are attached form carbonyl, C₃₋₈ carbocyclyl or 3-8 membered heterocyclyl.

According to some embodiments of the present disclosure, R₁₉/R₁₉ₐ together with the atoms to which they are attached form carbonyl, C₃₋₈ carbocyclyl or 3-8 membered heterocyclyl.

According to some embodiments of the present disclosure, R₂₀/R₂₀ₐ together with the atoms to which they are attached form carbonyl, C₃₋₈ carbocyclyl or 3-8 membered heterocyclyl.

According to some embodiments of the present disclosure, R₂₁/R₂₁ₐ together with the atoms to which they are attached form carbonyl, C₃₋₈ carbocyclyl or 3-8 membered heterocyclyl.

According to some embodiments of the present disclosure, R₁₈/R₁₉ together with the atoms to which they are attached form C₃₋₈ carbocyclyl.

According to some embodiments of the present disclosure, R₂₀/R₂₁ together with the atoms to which they are attached form C₃₋₈ carbocyclyl.

According to some embodiments of the present disclosure, R₁₈ and R₂₀ together form

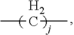

and j is selected from 0, 1 or 2.

According to some embodiments of the present disclosure, R₁₉ and R₂₁ together form

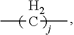

and j is selected from 0, 1 or 2.

According to some embodiments of the present disclosure, R₁₉ and R₂₀ together form

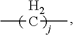

and j is selected from 0, 1 or 2.

According to some embodiments of the present disclosure, Y₃ is selected from

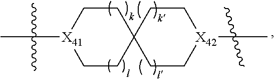

X₄₁ and X₄₂ are each independently selected from: CR₂₅R₂₅ₐ, NR₂₆, O or S; k, k′, 1 and 1′ are each independently selected from: 0 or 1;

and R₂₅, R₂₅ₐ and R₂₆ are each independently selected from H, halogen, hydroxyl, cyano, nitro, C₁₋₈ alkyl, C₂₋₈ alkenyl, C₂₋₈ alkynyl, C₃₋₈ cycloalkyl,

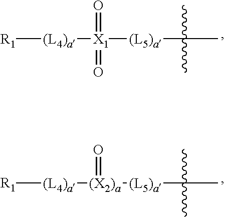

3-8 membered heterocyclyl, C₅₋₁₀ aryl or 5-10 membered heteroaryl.

According to some embodiments of the present disclosure, Y₃ is selected from

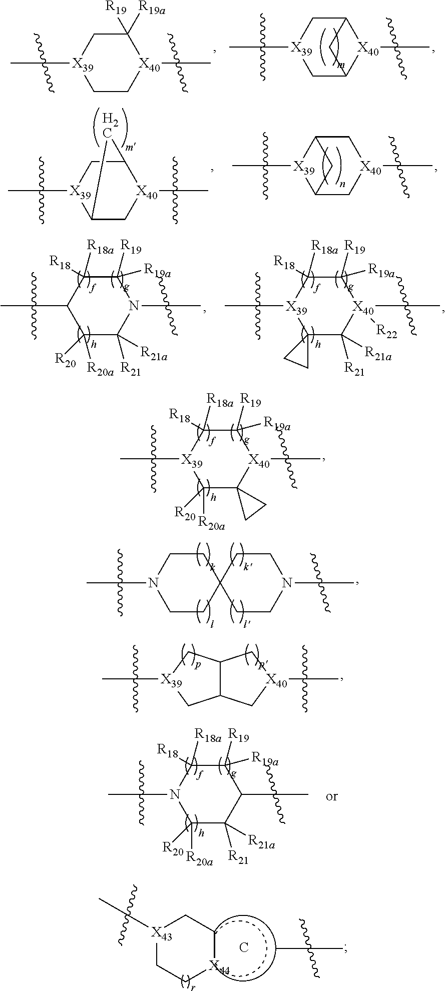

p, m, n, r and p′ are each independently selected from 0, 1 and 2; m′ is selected from 1 or 2;
X₄₃ and X₄₄ are each independently selected from: CR₂₇R₂₇ₐ, NR₂₈, O or S;
ring C is C₃₋₈ carbocyclyl, 3-8 membered heterocyclyl or C₅₋₁₀ aryl;
and R₂₇, R₂₇ₐ and R₂₈ are each independently selected from halogen, hydroxyl, cyano, nitro, C₁₋₈ alkyl, C₂₋₈ alkenyl, C₂₋₈ alkynyl, C₃₋₈ cycloalkyl,

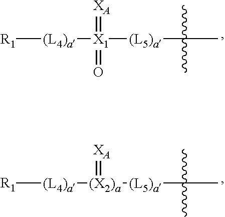

3-8 membered heterocyclyl, C₅₋₁₀ aryl or 5-10 membered heteroaryl.

According to some embodiments of the present disclosure, R₁₉ and R₁₉ₐ are each independently selected from H, ═O, halogen or C₁₋₈ alkyl, and R₁₉ and R₁₉ₐ are not simultaneously H.

According to some embodiments of the present disclosure, R₁₉ and R₁₉ₐ together with the carbon atoms to which they are attached form carbonyl.

According to some embodiments of the present disclosure, Y₃ is selected from

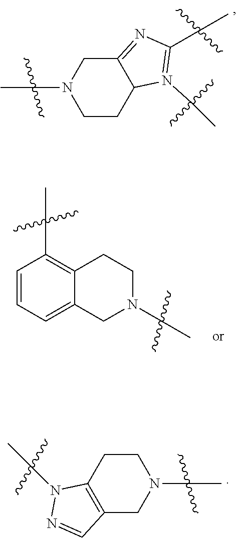

According to some embodiments of the present disclosure, Y₃ is selected from

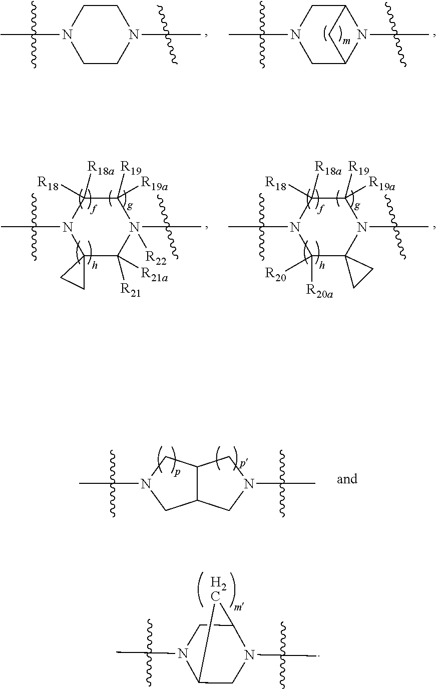

According to some embodiments of the present disclosure, Y₄ is selected from: 5-10 membered heteroaryl, C₂₋₈ alkenyl, C₂₋₈ alkynyl and C₅₋₁₀ aryl; and Y₄ is optionally substituted with 0-5 groups selected from: halogen, ═O, hydroxyl, cyano, nitro, C₁₋₈ alkyl, C₁₋₈ haloalkyl, C₁₋₈ alkoxy, C₂₋₈ alkenyl, C₂₋₈ alkynyl, C₃₋₈ cycloalkyl,

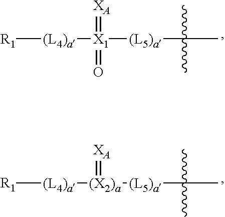

3-8 membered heterocyclyl, C₅₋₁₀ aryl or 5-10 membered heteroaryl.

According to some embodiments of the present disclosure, Y₄ is substituted with one or more substituents selected from: CN, hydroxyl, ═O, halogen, C₁₋₈ alkyl, C₁₋₈ haloalkyl, C₁₋₈ alkoxy, 3-8 membered heterocyclyl, C₃₋₈ cycloalkyl, C₂₋₈ alkenyl or C₂₋₈ alkynyl.

According to some embodiments of the present disclosure, Y₅ is selected from

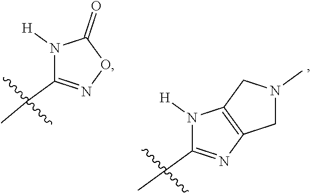

6 membered heteroaryl, 3-8 membered heterocyclyl, C₁₋₈ alkoxyphosphoryl, C₁₋₈ alkoxyacylamino, cyanoacylamino, triazole or tetrazole.

According to some embodiments of the present disclosure, Y₅ is optionally substituted with 0-5 substituents selected from: C₁₋₈ alkyl, ═O, C₁₋₆ alkoxy, C₁₋₈ haloalkyl, CN, 3-8 membered heterocyclyl, C₃₋₈ cycloalkyl, acylamino, C₅₋₁₀ aryl or 5-10 membered heteroaryl.

According to some embodiments of the present disclosure, the above-mentioned triazole or tetrazole is optionally substituted with 1-5 substituents selected from: C₁₋₈ alkyl, C₁₋₈ haloalkyl, 3-8 membered heterocyclyl, C₃₋₈ cycloalkyl, acylamino, C₅₋₁₀ aryl and 5-10 membered heteroaryl.

According to some embodiments of the present disclosure, L₁ is selected from a bond, C₂₋₈ alkynyl, C₂₋₈ alkenyl,

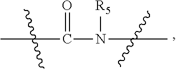

—N═N—, cyclopropyl, cyclobutyl, cyclopentyl, cyclohexyl, furyl, pyrrolyl or thiazolyl.

According to some embodiments of the present disclosure, R₅ is selected from H, halogen, C₁₋₈ alkyl or C₁₋₈ haloalkyl.

According to some embodiments of the present disclosure, L₂ is selected from a bond,

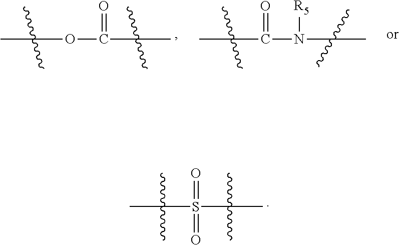

According to some embodiments of the present disclosure, L₃ is absent.

According to some embodiments of the present disclosure, L₃ is selected from a bond,

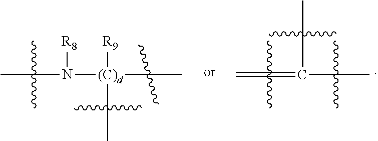

According to some embodiments of the present disclosure, R₈ and R₉ are each independently selected from H, halogen, C₁₋₈ alkyl or C₁₋₈ haloalkyl.

According to some embodiments of the present disclosure, L₃ is

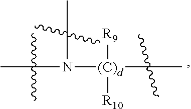

wherein the N-terminal thereof is connected to Y₄, and the C-terminal thereof is connected to Y₅;
and according to some embodiments of the present disclosure, R₈, R₉ and R₁₀ are each independently selected from H, halogen, C₁₋₆ alkyl or C₁₋₆ haloalkyl.

According to some embodiments of the present disclosure, R₂₂ is selected from halogen, hydroxyl, CN, C₁₋₈ alkyl or C₁₋₈ haloalkyl.

According to some embodiments of the present disclosure, Y₁ is selected from 5-10 membered heteroaryl, C₅₋₁₀ aryl or 5-8 membered heterocyclyl, wherein the groups are optionally substituted with 0-5 substituents selected from: halogen, hydroxyl, sulfhydryl, cyano, nitro, C₁₋₈ alkyl, C₂₋₈ alkenyl, C₂₋₈ alkynyl, C₃₋₈ cycloalkyl, 3-8 membered heterocyclyl, ═O, C₁₋₈ alkylacylamino, C₃₋₈ cycloalkylacylamino, 3-8 membered heterocyclyl, carboxamido, C₁₋₈ alkylaminoacyl, C₃₋₈ cycloalkyloxyacylamino, C₃₋₈ cycloalkylamino, C₁₋₈ haloalkylacylamino, 3-8 membered heterocyclylacylamino, C₃₋₈ cycloalkylacylamino, C₁₋₈ alkoxyacylamino, C₁₋₈ alkylaminoacylamino, 5-10 membered heteroaryl, 3-8 membered heterocyclyloxy, C₃-8 cycloalkylsulfonamido, C₁₋₈ alkylaminoacyloxy, C₁₋₈ alkylacyl, C₃₋₈ cycloalkyloxy and 3-8 membered heterocyclyloxyacylamino, wherein the above-mentioned substituents are optionally further substituted with 0-5 groups selected from: carbonyl, CN, C₁₋₈ alkylacyl, OH, C₃₋₈ cycloalkyl, C₁₋₈ alkyl, halogen or C₁₋₈ haloalkyl;

Y₂ is absent or selected from C₅₋₁₀ aryl or 5-10 membered heteroaryl, wherein Y₂ is optionally substituted with 0-5 substituents selected from: CN, C₁₋₈ haloalkyl, C₁₋₈ alkyl or C₃₋₈ cycloalkyl;
Y₃ is selected from: C₃₋₈ carbocyclyl, 3-8 membered heterocyclyl, C₅₋₁₀ aryl or 5-10 membered heteroaryl; preferably, Y₃ is selected from

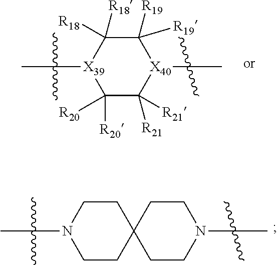

Y₃ is optionally further substituted with 0-5 substituents selected from: halogen, hydroxyl, C₁₋₈ alkyl, C₃₋₈ cycloalkyl, ═O and CN;
Y₄ is selected from C₅₋₁₀ aryl, 5-10 membered heteroaryl, C₂₋₈ alkenyl and C₂₋₈ alkynyl, wherein Y₄ is optionally substituted with 0-5 substituents selected from: CN, halogen, hydroxyl, ═O, C₁₋₈ alkyl, C₁₋₈ haloalkyl, C₁₋₈ alkoxy, 3-8 membered heterocyclyl, C₃₋₈ cycloalkyl or C₂₋₈ alkenyl;
Y₅ is selected from 3-8 membered heterocyclyl, 5-10 membered heteroaryl,

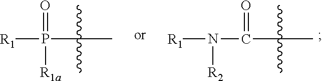

preferably, Y₅ is selected from C₁₋₈ alkoxyphosphoryl, C₁₋₈ alkoxyaminoacyl or cyanoaminoacyl; and Y₅ is optionally further substituted with one or more substituents selected from: C₁₋₈ alkyl, C₁₋₈ haloalkyl, 3-8 membered heterocyclyl, C₃₋₈ cycloalkyl, ═O, CN or C₁₋₈ alkoxy.

The present disclosure provides a compound represented by formula (VI), an isomer thereof or a pharmaceutically acceptable salt thereof:

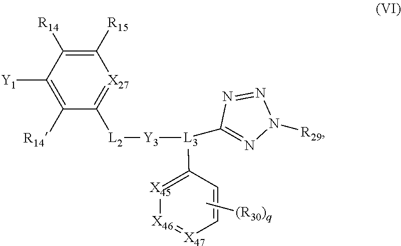

wherein,
R₂₉ is selected from: H, C₁₋₈ haloalkyl, 3-8 membered heterocyclyl, C₃₋₈ cycloalkyl or C₁₋₈ haloalkyl;
X₄₅, X₄₆ and X₄₇ are each independently selected from: CR₃₁ or N;
and R₃₀ and R₃₁ are each independently selected from: H, CN, halogen, C₁₋₈ alkyl, C₁₋₈ haloalkyl, C₁₋₈ alkoxy, 3-8 membered heterocyclyl, C₃₋₈ cycloalkyl or C₂₋₈ alkenyl.

According to a particular embodiment of the present disclosure, Y₁ is selected from:

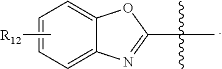

According to a particular embodiment of the present disclosure, Y₂ is selected from:

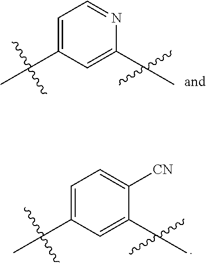 [SMILES: *c1ccnc(*)c1 ; *c1ccc(C#N)c(*)c1]

According to a particular embodiment of the present disclosure, Y₃ is selected from:

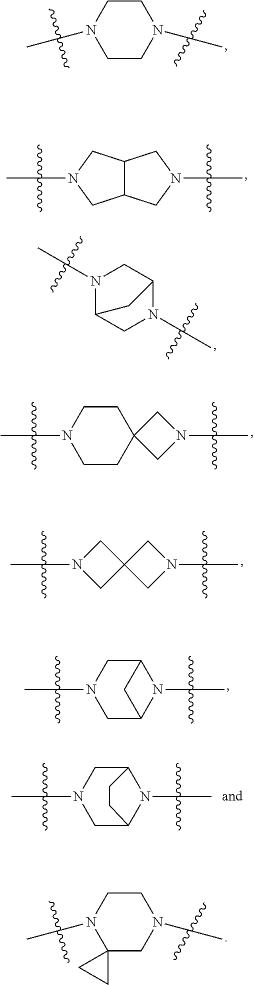

According to a particular embodiment of the present disclosure, Y₄ is selected from: phenyl, o-fluorophenyl, m-fluorophenyl, p-fluorophenyl,

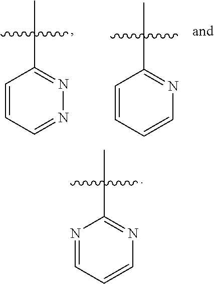 [SMILES: *c1cccnn1 ; *c1ccccn1 ; *c1ncccn1]

According to a particular embodiment of the present disclosure, Y₅ is selected from:

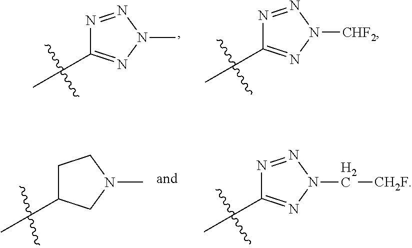

According to a particular embodiment of the present disclosure, L₁ is selected from: a bond.

According to a particular embodiment of the present disclosure, L₂ is selected from: carbonyl.

According to a particular embodiment of the present disclosure, L₃ is selected from: CH.

The present disclosure relates to a compound represented by formula (VII), an isomer thereof or a pharmaceutically acceptable salt thereof:

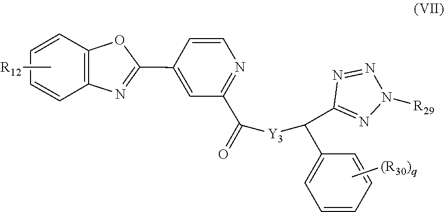

wherein the definition of each substituent is the same as before.

In certain embodiments, R¹² in the compound represented by formula (VII), the isomer thereof or the pharmaceutically acceptable salt thereof is selected from C₁₋₈ alkoxy, 3-8 membered heterocyclyl, C₁₋₆ alkoxy-C(O)—NH—, C₁₋₆ alkyl-C(O)—NH— or C₃₋₈ cycloalkyl-C(O)—NH—, wherein the above-mentioned groups are optionally further substituted with 0-3 groups selected from: halogen or 3-8 membered heterocyclyl; in certain embodiments, R¹² is selected from

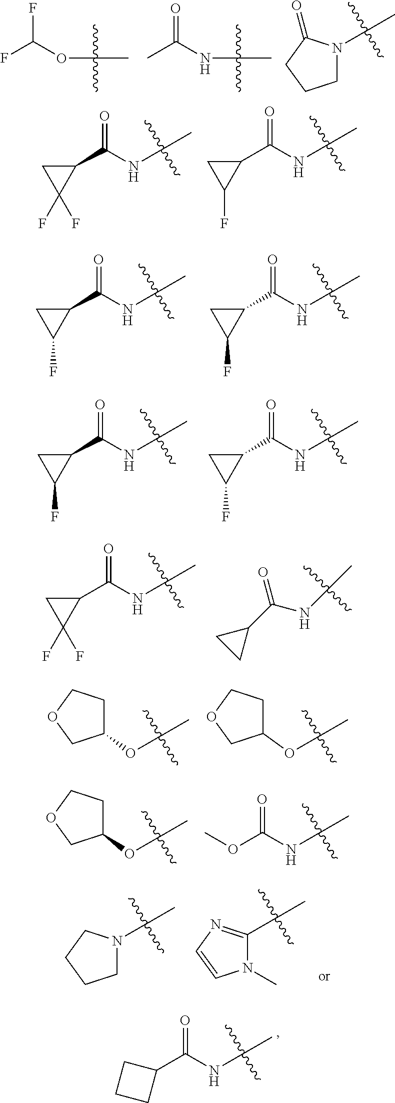

or R¹² is selected from

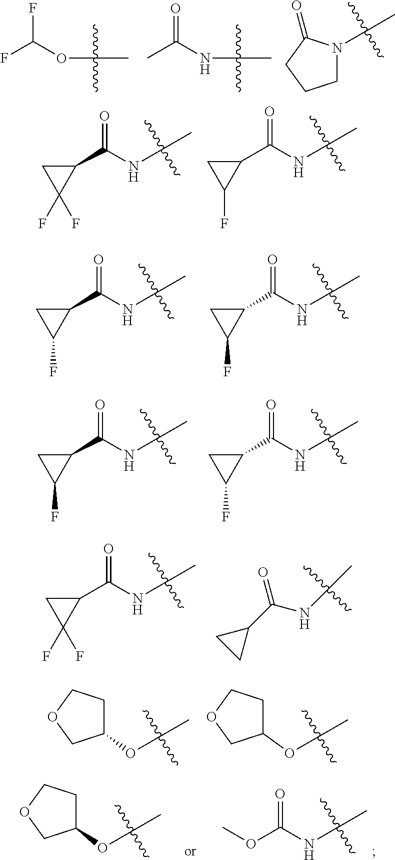

in certain embodiments, R¹² is selected from

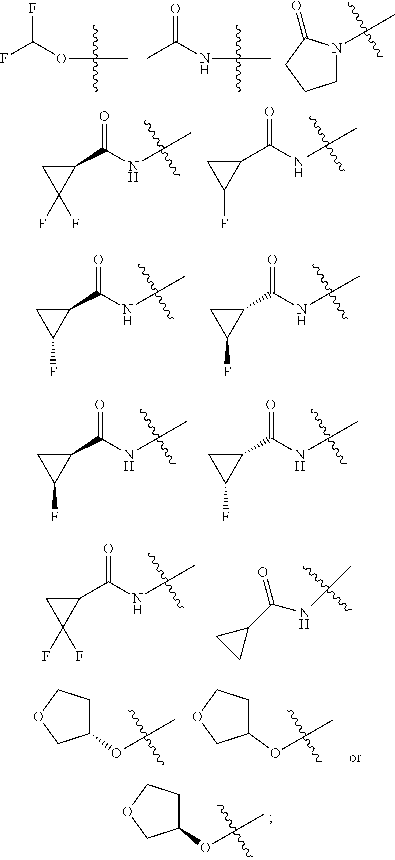

Y₃ is selected from piperazinylene or

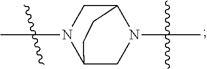 [SMILES: *N1CC2CCC1CN2*]

in certain embodiments, Y₃ is selected from piperazinylene;
and R²⁹ is selected from C₁₋₆ alkyl or halo C₁₋₆ alkyl; in certain embodiments, R²⁹ is selected from methyl, fluoroethyl or difluoromethyl; R³⁰ is selected from H or halogen; and in certain embodiments, R³⁰ is selected from H.

The present disclosure further discloses a compound represented by formula (VIII), an isomer thereof or a pharmaceutically acceptable salt thereof:

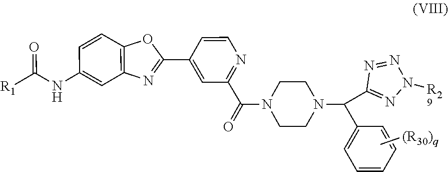

wherein the definition of each group is the same as before.

Embodiment one: the present disclosure discloses a compound of formula (I), an isomer thereof or a pharmaceutically acceptable salt thereof,

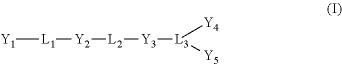

wherein Y₁ is selected from

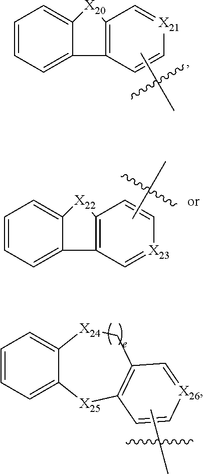

wherein the above-mentioned groups are optionally substituted with 0-5 substituents selected from: halogen, ═O, hydroxyl, cyano, nitro, silyl, C₁₋₈ alkyl, C₁₋₈ alkoxy, C₂₋₈ alkenyl, C₂₋₈ alkynyl, C₃₋₈ cycloalkyl,

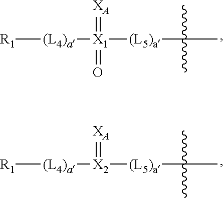

membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₂₀, X₂₂, X₂₄ and X₂₅ are each independently selected from a bond, C, O, NR₂ or S;
e is 0 or 1;
X₂₁, X₂₃ and X₂₆ are selected from C or NR₂; [symbol]
represents a single bond or a double bond;
Y₂, Y₃, Y₄ and Y₅ are each independently selected from: C₃₋₁₂ carbocyclyl, C₅₋₁₅ aryl, 5-15 membered heteroaryl, 3-12 membered heterocyclyl, C₂₋₈ alkenyl, C₂₋₈ alkynyl,

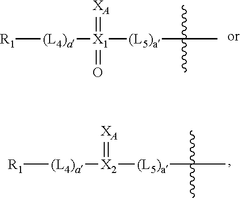

wherein
the above-mentioned groups are optionally substituted with 0-5 substituents selected from: halogen, ═O, hydroxyl, cyano, nitro, silyl, C₁₋₈ alkyl, C₁₋₈ alkoxy, C₂₋₈ alkenyl, C₂₋₈ alkynyl, C₃₋₈ cycloalkyl,

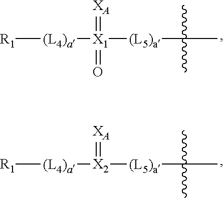

3-8 membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₁ is selected from P or S;
X₂ is selected from C, PR₁ₐ or S;
L₄ and L₅ are each independently selected from: a bond, NR₂, CR₃R₄, O, or S;
X_{A} is selected from C, NR₂ₐ, O and S; a is 0 or 1;
a′ is selected from 0, 1, 2, 3, 4, 5 or 6;
R₁, R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the above-mentioned substituents are optionally further substituted with 0-5 groups selected from: halogen, C₁₋₈ alkyl, C₁₋₈ alkoxy or C₁₋₈ haloalkyl; or R₁ is absent;
or Y₂ is absent;
L₁ is absent or selected from: a bond, C₂₋₈ alkynyl, C₂₋₈ alkenyl,

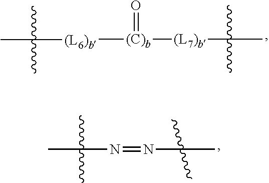

3-8 membered heterocyclyl, C₃₋₈ cycloalkyl, C₅₋₁₀ aryl or 5-10 membered heteroaryl, wherein the alkynyl, alkenyl,

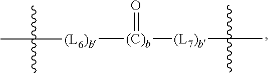

heterocyclyl, cycloalkyl, aryl or heteroaryl is optionally substituted with 0-5 groups selected from: hydroxyl, halogen, C₁₋₈ alkyl or C₁₋₈ haloalkyl;
L₂ is absent or selected from a bond,

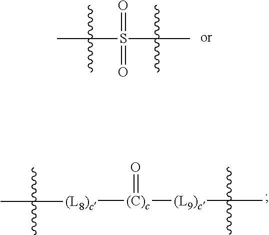

L₆, L₇, L₈ and L₉ are each independently selected from a bond, O, S, NR₅ or CR₆R₇;
b and c are each independently selected from 0 or 1; b′ and c′ are each independently selected from 0, 1, 2, 3, 4, 5 or 6;
R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl;
L₃ is absent or selected from: a bond,

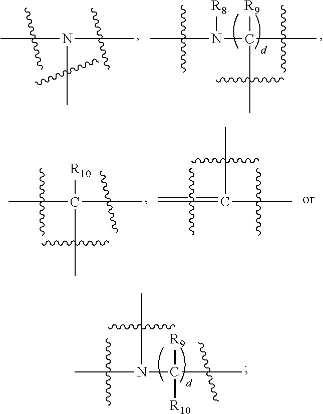

d is an integer selected from 1-6;
and R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl.

In certain embodiments, Y₁ is selected from

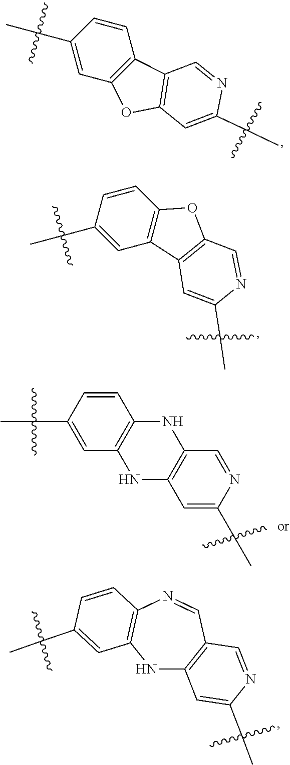 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

wherein the above-mentioned groups are optionally substituted with 0-5 substituents selected from: acetamido, and the definitions of other groups are consistent with those of embodiment one.

In certain embodiments, L₁ is selected from a bond, and the definitions of other groups are consistent with those of embodiment one.

In certain embodiments, L₂ is selected from

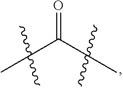 [SMILES: *C(*)=O]

and the definitions of other groups are consistent with those of embodiment one.

In certain embodiments, L₃ is selected from

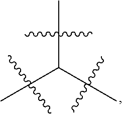 [SMILES: *C(*)*]

and the definitions of other groups are consistent with those of embodiment one.

In certain embodiments, Y₂ is selected from phenyl or

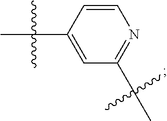 [SMILES: *c1ccnc(*)c1]

in certain embodiments, Y₂ is selected from phenyl; in certain embodiments, Y₂ is selected from

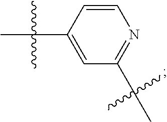 [SMILES: *c1ccnc(*)c1]

and the definitions of other groups are consistent with those of embodiment one.

In certain embodiments, Y₃ is selected from

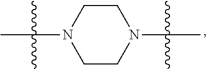 [SMILES: *N1CCN(*)CC1]

and the definitions of other groups are consistent with those of embodiment one.

In certain embodiments, Y₄ is selected from phenyl, and the definitions of other groups are consistent with those of embodiment one.

In certain embodiments, Y₅ is selected from

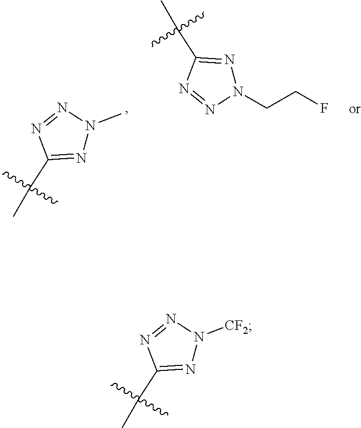

in certain embodiments, Y₅ is selected from

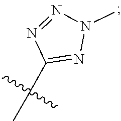 [SMILES: *c1nnn(C)n1]

in certain embodiments, Y₅ is selected from

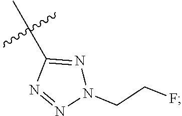 [SMILES: *c1nnn(CCF)n1]

and in certain embodiments, Y₅ is selected from

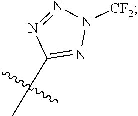

and the definitions of other groups are consistent with those of embodiment one.

In certain embodiments, L₁ is selected from a bond; L₂ is selected from

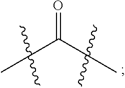 [SMILES: *C(*)=O]

L₃ is selected from

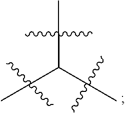 [SMILES: *C(*)*]

Y₂ is selected from phenyl or

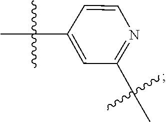 [SMILES: *c1ccnc(*)c1]

Y₃ is selected from

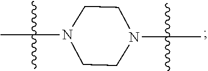 [SMILES: *N1CCN(*)CC1]

Y₄ is selected from phenyl; Y₅ is selected from

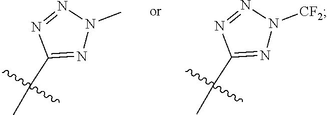

and the definition of Y₁ is consistent with that of embodiment one.

In certain embodiments, L₁ is selected from a bond; L₂ is selected from

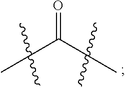 [SMILES: *C(*)=O]

L₃ is selected from

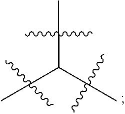 [SMILES: *C(*)*]

Y₂ is selected from

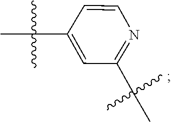 [SMILES: *c1ccnc(*)c1]

Y₃ is selected from

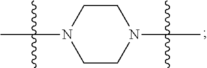 [SMILES: *N1CCN(*)CC1]

Y₄ is selected from phenyl; Y₅ is selected from

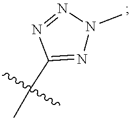 [SMILES: *c1nnn(C)n1]

and Y₁ is selected from

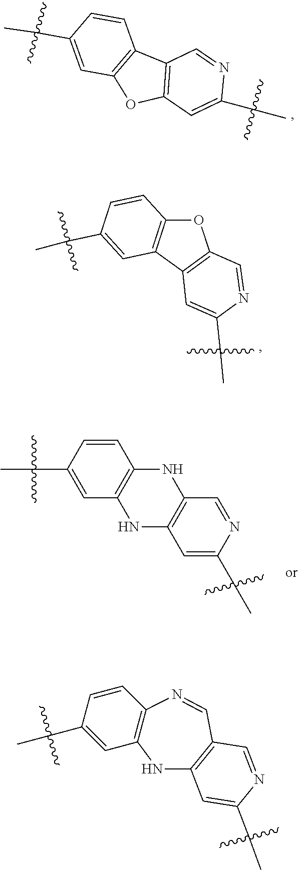 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

with 0-5 substituents selected from: acetamido.

Embodiment two: the present disclosure discloses a compound of formula (I), an isomer thereof or a pharmaceutically acceptable salt thereof,

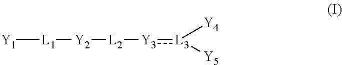

wherein Y₃ is selected from:

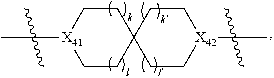

wherein the above-mentioned groups are optionally substituted with 0-5 substituents selected from: halogen, ═O, hydroxyl, cyano, nitro, silyl, C₁₋₈ alkyl, C₁₋₈ alkoxy, C₂₋₈ alkenyl, C₂₋₈ alkynyl, C₃₋₈
cycloalkyl,

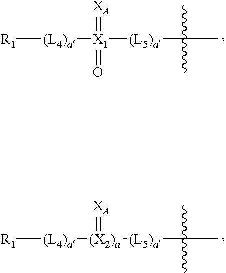

3-8 membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₄₁ and X₄₂ are each independently selected from: CR₂₅R₂₅ₐ, NR₂₆, O or S;
k, k′, 1 and 1′ are each independently selected from: 0 or 1;
R₂₅, R₂₅ₐ and R₂₆ are each independently selected from H, halogen, hydroxyl, cyano, nitro, C₁₋₈ alkyl, C₂₋₈ alkenyl, C₂₋₈ alkynyl, C₃₋₈ cycloalkyl,

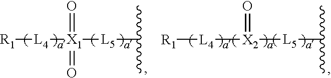

3-8 membered heterocyclyl, C₅₋₁₀ aryl or 5-10 membered heteroaryl; [symbol]
represents a single bond or a double bond;
Y₁, Y₂, Y₄ and Y₅ are each independently selected from: C₃₋₁₂ carbocyclyl, C₅₋₁₅ aryl, 5-15 membered heteroaryl, 3-12 membered heterocyclyl, C₂₋₈ alkenyl, C₂₋₈ alkynyl,

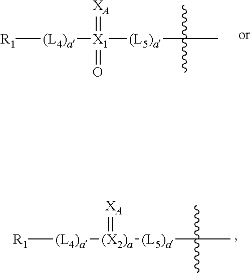

wherein the above-mentioned groups are optionally substituted with 0-5 substituents selected from: halogen, ═O, hydroxyl, cyano, nitro, silyl, C₁₋₈ alkyl, C₁₋₈ alkoxy, C₂₋₈ alkenyl, C₂₋₈ alkynyl, C₃₋₈ cycloalkyl,

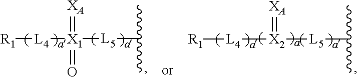

3-8 membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₁ is selected from P or S;
X₂ is selected from C, PR₁ₐ or S;
L₄ and L₅ are each independently selected from: a bond, NR₂, CR₃R₄, O, or S;
X_{A} is selected from C, NR₂ₐ, O and S; a is 0 or 1;
a′ is selected from 0, 1, 2, 3, 4, 5 or 6;
R₁, R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the above-mentioned substituents are optionally further substituted with 0-5 groups selected from: halogen, C₁₋₈ alkyl, C₁₋₈ alkoxy or C₁₋₈ haloalkyl; or R₁ is absent;
or Y₂ is absent;
L₁ is absent or selected from: a bond, C₂₋₈ alkynyl, C₂₋₈ alkenyl,

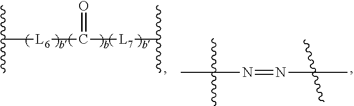

3-8 membered heterocyclyl, C₃₋₈ cycloalkyl, C₅₋₁₀ aryl or 5-10 membered heteroaryl, wherein the alkynyl, alkenyl,

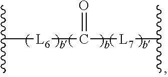

heterocyclyl, cycloalkyl, aryl or heteroaryl is optionally substituted with 0-5 groups selected from: hydroxyl, halogen, C₁₋₈ alkyl or C₁₋₈ haloalkyl;
L₂ is absent or selected from a bond,

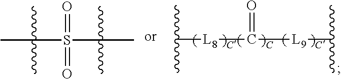

L₆, L₇, L₈ and L₉ are each independently selected from a bond, O, S, NR₅ or CR₆R₇;
b and c are each independently selected from 0 or 1; b′ and c′ are each independently selected from 0, 1, 2, 3, 4, 5 or 6;
R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl;
L₃ is absent or selected from: a bond,

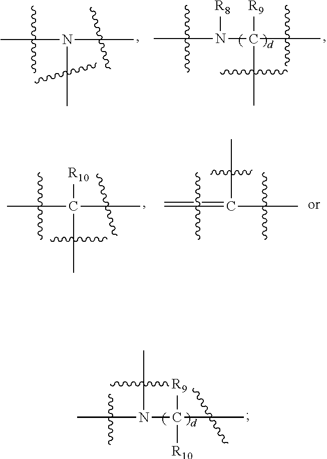

d is an integer selected from 1-6;
and R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl.

In certain embodiments, L₁ is selected from a bond, and the definitions of other groups are consistent with those of embodiment two.

In certain embodiments, L₂ is selected from

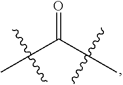 [SMILES: *C(*)=O]

and the definitions of other groups are consistent with those of embodiment two.

In certain embodiments, L₃ is selected from

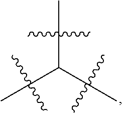 [SMILES: *C(*)*]

and the definitions of other groups are consistent with those of embodiment two.

In certain embodiments, Y₂ is selected from phenyl or

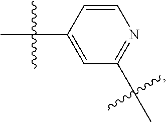 [SMILES: *c1ccnc(*)c1]

and the definitions of other groups are consistent with those of embodiment two.

In certain embodiments, Y₁ is selected from

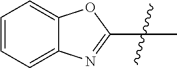 [SMILES: *c1nc2ccccc2o1]

and optionally substituted with acetamido, cyclopropylcarboxamido or

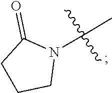 [SMILES: *N1CCCC1=O]

in certain embodiments, Y₁ is optionally substituted with acetamido; in certain embodiments, Y₁ is optionally substituted with cyclopropylcarboxamido; in certain embodiments, Y₁ is optionally substituted with

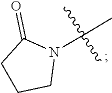 [SMILES: *N1CCCC1=O]

and the definitions of other groups are consistent with those of embodiment two.

In certain embodiments, Y₄ is selected from phenyl, and the definitions of other groups are consistent with those of embodiment two.

In certain embodiments, Y₅ is selected from

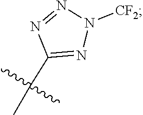

in certain embodiments, Y₅ is selected from

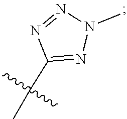 [SMILES: *c1nnn(C)n1]

in certain embodiments, Y₅ is selected from

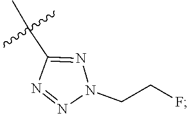 [SMILES: *c1nnn(CCF)n1]

in certain embodiments, Y₅ is selected from

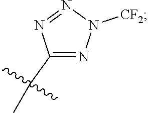

and the definitions of other groups are consistent with those of embodiment two.

In certain embodiments, L₁ is selected from a bond; L₂ is selected from

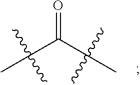 [SMILES: *C(*)=O]

L₃ is selected from

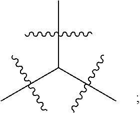 [SMILES: *C(*)*]

Y₂ is selected from phenyl or

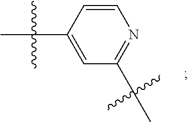 [SMILES: *c1ccnc(*)c1]

Y₄ is selected from phenyl; Y₅ is selected from

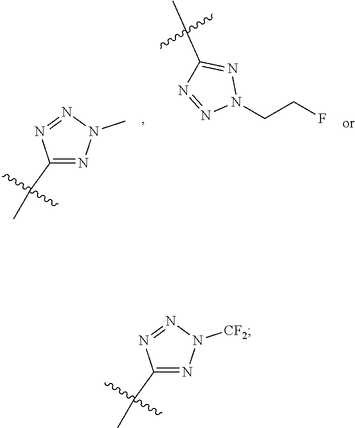

and the definitions of other groups are consistent with those of embodiment two.

In certain embodiments, L₁ is selected from a bond; L₂ is selected from

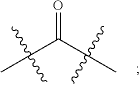 [SMILES: *C(*)=O]

L₃ is selected from

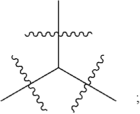 [SMILES: *C(*)*]

Y₂ is selected from

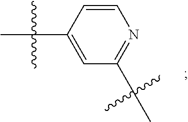 [SMILES: *c1ccnc(*)c1]

Y₄ is selected from phenyl; Y₅ is selected from

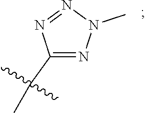 [SMILES: *c1nnn(C)n1]

Y₁ is selected from

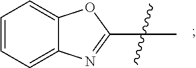 [SMILES: *c1nc2ccccc2o1]

Y₁ is optionally substituted with acetamido, cyclopropylcarboxamido or

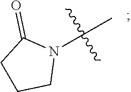 [SMILES: *N1CCCC1=O]

and the definition of Y₃ is consistent with that of scheme two.

In certain embodiments, Y₃ is selected from

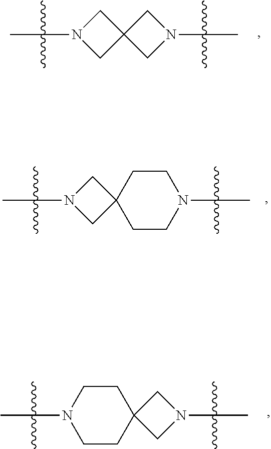 [SMILES: *N1CC2(C1)CN(*)C2 ; *N1CCC2(CC1)CN(*)C2 ; *N1CCC2(CC1)CN(*)C2]

and the definitions of other groups are consistent with those of embodiment two.

In certain embodiments, Y₃ is selected from

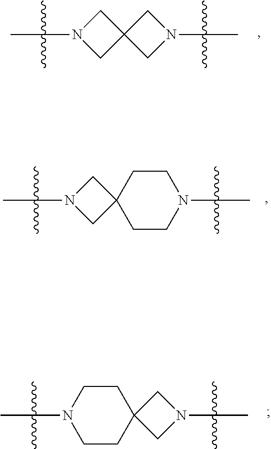 [SMILES: *N1CC2(C1)CN(*)C2 ; *N1CCC2(CC1)CN(*)C2 ; *N1CCC2(CC1)CN(*)C2]

L₁ is selected from a bond; L₂ is selected from

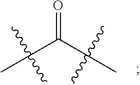 [SMILES: *C(*)=O]

L₃ is selected from

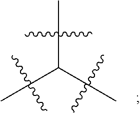 [SMILES: *C(*)*]

Y₂ is selected from

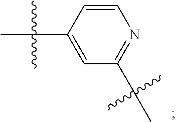 [SMILES: *c1ccnc(*)c1]

Y₄ is selected from phenyl; Y₅ is selected from

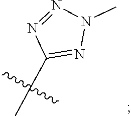 [SMILES: *c1nnn(C)n1]

Y₁ is selected from

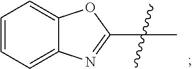 [SMILES: *c1nc2ccccc2o1]

and Y₁ is optionally substituted with 0-5 of the following groups: C₃₋₈ cycloalkyl,

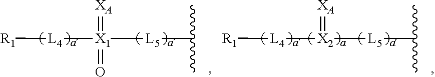

3-8 membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl.

In certain embodiments, Y₃ is selected from

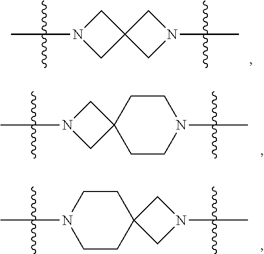

L₁ is selected from a bond; L₂ is selected from

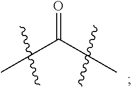 [SMILES: *C(*)=O]

L₃ is selected from

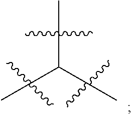 [SMILES: *C(*)*]

Y₂ is selected from

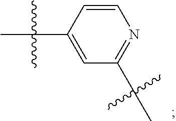 [SMILES: *c1ccnc(*)c1]

Y₄ is selected from phenyl; Y₅ is selected from

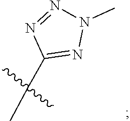 [SMILES: *c1nnn(C)n1]

Y₁ is selected from

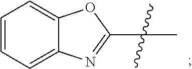 [SMILES: *c1nc2ccccc2o1]

and Y₁ is optionally substituted with 0-3 of the following groups: acetamido.

In the compound of formula (I), the isomer thereof or the pharmaceutically acceptable salt thereof in embodiment two of the present disclosure, Y₃ also can be selected from

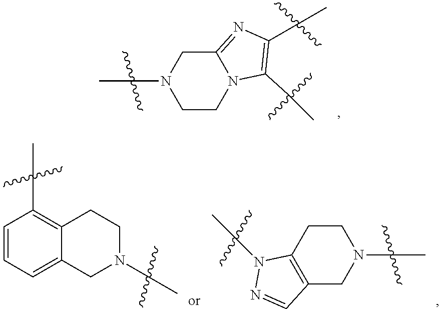 [SMILES: *c1nc2n(c1*)CCN(*)C2 ; *c1cccc2c1CCN(*)C2 ; *N1CCc2c(cnn2*)C1]

and other groups are consistent with those of embodiment two.

In certain embodiments, Y₃ is selected from

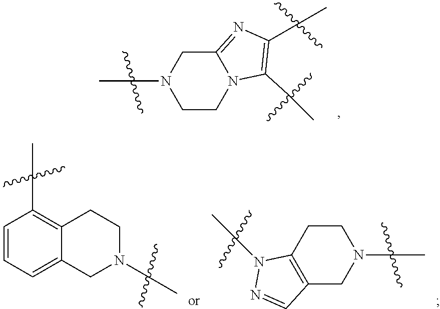 [SMILES: *c1nc2n(c1*)CCN(*)C2 ; *c1cccc2c1CCN(*)C2 ; *N1CCc2c(cnn2*)C1]

L₁ is selected from a bond; L₂ is selected from

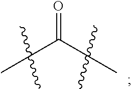 [SMILES: *C(*)=O]

L₃ is selected from

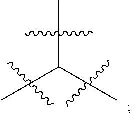 [SMILES: *C(*)*]

Y₂ is selected from

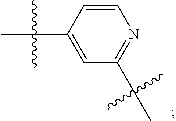 [SMILES: *c1ccnc(*)c1]

Y₄ is selected from phenyl; Y₅ is selected from

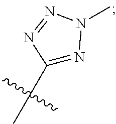 [SMILES: *c1nnn(C)n1]

Y₁ is selected from N

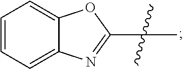 [SMILES: *c1nc2ccccc2o1]

and Y₁ is optionally substituted with 0-3 of the following groups: C₃₋₈ cycloalkyl,

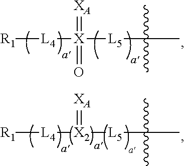

3-8 membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl.

In certain embodiments, Y₃ is selected from

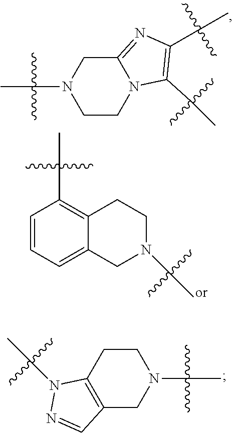 [SMILES: *c1nc2n(c1*)CCN(*)C2 ; *c1cccc2c1CCN(*)C2 ; *N1CCc2c(cnn2*)C1]

L₁ is selected from a bond; L₂ is selected from

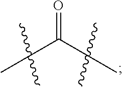 [SMILES: *C(*)=O]

L₃ is selected from

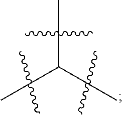 [SMILES: *C(*)*]

Y₂ is selected from

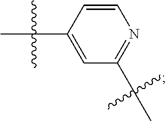 [SMILES: *c1ccnc(*)c1]

Y₄ is selected from phenyl; Y₅ is selected from

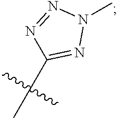 [SMILES: *c1nnn(C)n1]

Y₁ is selected from

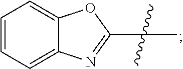 [SMILES: *c1nc2ccccc2o1]

and Y₁ is optionally substituted with 0-3 of the following groups: acetamido.

Embodiment three: the present disclosure relates to a compound of formula (I), an isomer thereof or a pharmaceutically acceptable salt thereof,

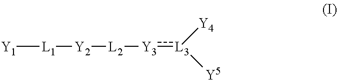

wherein L₃ is

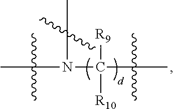

wherein the N-terminal thereof is connected to Y₄, and the C-terminal thereof is connected to Y₅;
R₉ and R₁₀ are each independently selected from H, halogen, C₁₋₆ alkyl or C₁₋₆ haloalkyl; [symbol]
represents a single bond or a double bond;
Y₁, Y₂, Y₃, Y₄ and Y₅ are each independently selected from: C₃₋₁₂ carbocyclyl, C₅₋₁₅ aryl, 5-15 membered heteroaryl, 3-12 membered heterocyclyl, C₂₋₈ alkenyl, C₂₋₈ alkynyl,

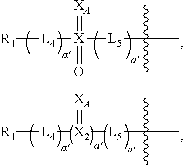

wherein
the above-mentioned groups are optionally substituted with 0-5 substituents selected from: halogen, ═O, hydroxyl, cyano, nitro, silyl, C₁₋₈ alkyl, C₁₋₈ alkoxy, C₂₋₈ alkenyl, C₂₋₈ alkynyl, C₃₋₈ cycloalkyl,

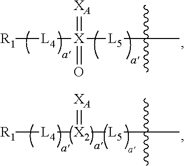

3-8 membered heterocyclyl, C₅₋₁₀ aryl or 5-10 membered heteroaryl, wherein the above-mentioned substituents are optionally further substituted with 0-5 groups selected from: halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₁ is selected from P or S;
X₂ is selected from C, PR₁ₐ or S;
L₄ and L₅ are each independently selected from: a bond, NR₂, CR₃R₄, O, or S;
X_{A} is selected from C, NR₂ₐ, O and S; a is 0 or 1;
a′ is selected from 0, 1, 2, 3, 4, 5 or 6;
R₁, R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the above-mentioned substituents are optionally further substituted with 0-5 groups selected from: halogen, C₁₋₈ alkyl, C₁₋₈ alkoxy or C₁₋₈ haloalkyl; or R₁ is absent;
or Y₂ is absent;
L₁ is absent or selected from: a bond, C₂₋₈ alkynyl, C₂₋₈ alkenyl,

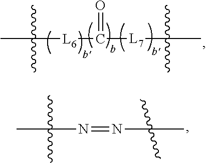

3-8 membered heterocyclyl, C₃₋₈ cycloalkyl, C₅₋₁₀ aryl or 5-10 membered heteroaryl, wherein the alkynyl, alkenyl,

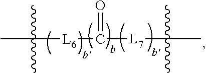

heterocyclyl, cycloalkyl, aryl or heteroaryl is optionally substituted with 0-5 groups selected from: hydroxyl, halogen, C₁₋₈ alkyl or C₁₋₈ haloalkyl;
L₂ is absent or selected from a bond,

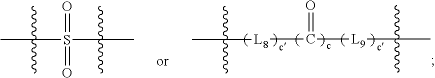

L₆, L₇, L₈ and L₉ are each independently selected from a bond, O, S, NR₅ or CR₆R₇; b and c are each independently selected from 0 or 1; b′ and c′ are each independently selected from 0, 1, 2, 3, 4, 5 or 6; R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl; and d is an integer selected from 1-6.

In certain embodiments, [symbol] represents a single bond, and other groups are consistent with those of embodiment three.

In certain embodiments, L₁ is selected from a bond, and other groups are consistent with those of embodiment three.

In certain embodiments, L₂ is selected from

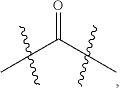 [SMILES: *C(*)=O]

and other groups are consistent with those of embodiment three.

In certain embodiments, Y₂ is selected from phenyl or

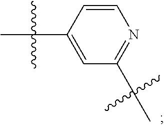 [SMILES: *c1ccnc(*)c1]

in certain embodiments, Y₂ is selected from phenyl; and in certain embodiments, Y₂ is selected from

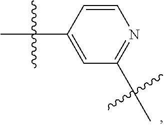 [SMILES: *c1ccnc(*)c1]

and other groups are consistent with those of embodiment three.

In certain embodiments, Y₁ is selected from

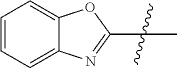 [SMILES: *c1nc2ccccc2o1]

and optionally substituted with acetamido; and other groups are consistent with those of embodiment three.

In certain embodiments, Y₄ is selected from phenyl, and other groups are consistent with those of embodiment three.

In certain embodiments, Y₅ is selected from

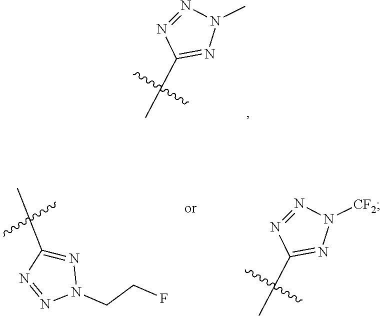

in certain embodiments, Y₅ is selected from

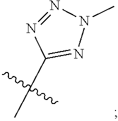 [SMILES: *c1nnn(C)n1]

in certain embodiments, Y₅ is selected from

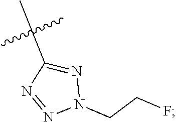 [SMILES: *c1nnn(CCF)n1]

and in certain embodiments, Y₅ is selected from

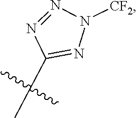

and other groups are consistent with those of embodiment three.

In certain embodiments, L₁ is selected from a bond; L₂ is selected from

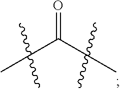 [SMILES: *C(*)=O]

Y₁ is selected from

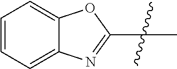 [SMILES: *c1nc2ccccc2o1]

and optionally substituted with acetamido; Y₂ is selected from phenyl or

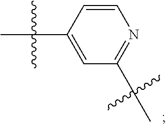 [SMILES: *c1ccnc(*)c1]

Y₃ is selected from

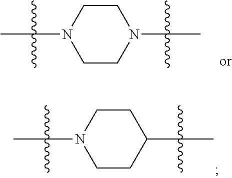 [SMILES: *N1CCN(*)CC1 ; *C1CCN(*)CC1]

Y₄ is selected from phenyl; Y₅ is selected from

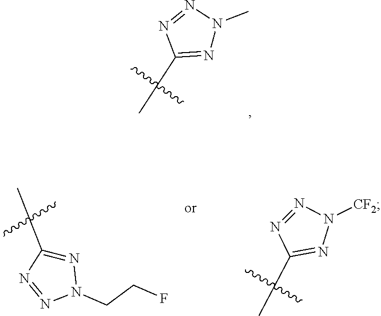

and the definition of L₃ is consistent with that of embodiment three.

In certain embodiments, L₃ is

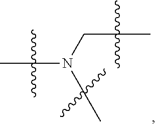 [SMILES: *CN(*)*]

wherein the N-terminal thereof is connected to Y₄, and the C-terminal thereof is connected to Y₅; and the definitions of other groups are consistent with those of embodiment three.

Embodiment four: the present disclosure relates to a compound represented by general formula (VII), an isomer thereof or a pharmaceutically acceptable salt thereof:

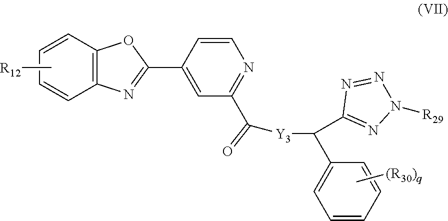

wherein R₁₂ is selected from C₁₋₈ alkoxy, 3-8 membered heterocyclyl, C₁₋₆ alkoxy-C(O)—NH—, C₁₋₆ alkyl-C(O)—NH— or C₃₋₈ cycloalkyl-C(O)—NH—, wherein the above-mentioned groups are optionally further substituted with 0-3 groups selected from: halogen or 3-8 membered heterocyclyl; in certain embodiments, R₁₂ is selected from C₁₋₈ alkoxy, 3-8 membered heterocyclyl, C₁₋₆ alkoxy-C(O)—NH—, C₁₋₆ alkyl-C(O)—NH— or C₃₋₈ cycloalkyl-C(O)—NH—, wherein the above-mentioned groups are optionally further substituted with 0-3 groups selected from: halogen; in certain embodiments, R₁₂ is selected from 3-8 membered heterocyclyl, C₁₋₆ alkyl-C(O)—NH— or C₃₋₈ cycloalkyl-C(O)—NH—, wherein the above-mentioned groups are optionally further substituted with 0-3 groups selected from: halogen; in certain embodiments, R₁₂ is selected from C₁₋₆ alkyl-C(O)—NH— or C₃₋₈ cycloalkyl-C(O)—NH—, wherein the above-mentioned groups are optionally further substituted with 0-3 groups selected from: halogen; in certain embodiments, R₁₂ is selected from C₃₋₈ cycloalkyl-C(O)—NH—, wherein the above-mentioned group is optionally further substituted with 0-3 groups selected from: halogen; in certain embodiments, R₁₂ is selected from C₁₋₆ alkyl-C(O)—NH—, wherein the above-mentioned group is optionally further substituted with 0-3 groups selected from: halogen; in certain embodiments, R₁₂ is selected from C₁₋₆ alkoxy-C(O)—NH—, wherein the above-mentioned group is optionally further substituted with 0-3 groups selected from: halogen; in certain embodiments, R₁₂ is selected from 3-8 membered heterocyclyl, wherein the above-mentioned group is optionally further substituted with 0-3 groups selected from: halogen; in certain embodiments, R₁₂ is selected from C₁₋₈ alkoxy, wherein the above-mentioned group is optionally further substituted with 0-3 groups selected from: halogen;
q is selected from 0, 1, 2, 3, 4 and 5; in certain embodiments, q is selected from 0 or 1; in certain embodiments, q is selected from 0;
Y₃ is selected from piperazinylene or

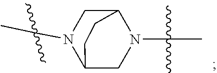 [SMILES: *N1CC2CCC1CN2*]

in certain embodiments, Y₃ is selected from piperazinylene; in certain embodiments, Y₃ is selected from

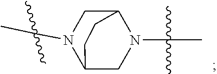 [SMILES: *N1CC2CCC1CN2*]

R₂₉ is selected from halo C₁₋₆ alkyl; in certain embodiments, R₂₉ is selected from difluoromethyl or fluoroethyl; in certain embodiments, R₂₉ is selected from difluoromethyl; in certain embodiments, R₂₉ is selected from fluoroethyl;
R₃₀ is selected from H or halogen; and in certain embodiments, R₃₀ is selected from H.

In certain embodiments, embodiment four of the present disclosure relates to a compound represented by general formula (VII), an isomer thereof or a pharmaceutically acceptable salt thereof:

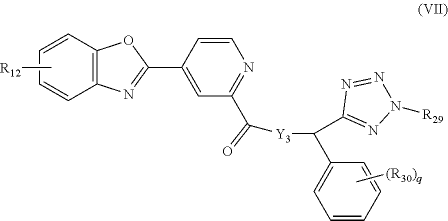

wherein R₁₂ is selected from C₁₋₈ alkoxy, 3-8 membered heterocyclyl, C₁₋₆ alkoxy-C(O)—NH—, C₁₋₆ alkyl-C(O)—NH— or C₃₋₈ cycloalkyl-C(O)—NH—, wherein the above-mentioned groups are optionally further substituted with 0-3 groups selected from: halogen or 3-8 membered heterocyclyl;
q is selected from 0, 1, 2, 3, 4 and 5;
Y₃ is selected from piperazinylene or

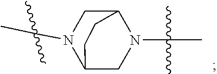 [SMILES: *N1CC2CCC1CN2*]

R₂₉ is selected from halo C₁₋₆ alkyl;
and R₃₀ is selected from H or halogen.

In certain embodiments, R₁₂ is selected from C₁₋₈ alkoxy, 3-8 membered heterocyclyl, C₁₋₆ alkoxy-C(O)—NH—, C₁₋₆ alkyl-C(O)—NH— or C₃₋₈ cycloalkyl-C(O)—NH—, wherein the above-mentioned groups are optionally further substituted with 0-3 groups selected from: halogen; in certain embodiments, R₁₂ is selected from 3-8 membered heterocyclyl, C₁₋₆ alkyl-C(O)—NH— or C₃₋₈ cycloalkyl-C(O)—NH—, wherein the above-mentioned groups are optionally further substituted with 0-3 groups selected from: halogen; in certain embodiments, R¹² is selected from C₁₋₆ alkyl-C(O)—NH— or C₃₋₈ cycloalkyl-C(O)—NH—, wherein the above-mentioned groups are optionally further substituted with 0-3 groups selected from: halogen; in certain embodiments, R₁₂ is selected from C₃₋₈ cycloalkyl-C(O)—NH—, wherein the above-mentioned group is optionally further substituted with 0-3 groups selected from: halogen; in certain embodiments, R₁₂ is selected from C₁₋₆ alkyl-C(O)—NH—, wherein the above-mentioned group is optionally further substituted with 0-3 groups selected from: halogen; in certain embodiments, R₁₂ is selected from C₁₋₆ alkoxy-C(O)—NH—, wherein the above-mentioned group is optionally further substituted with 0-3 groups selected from: halogen; in certain embodiments, R₁₂ is selected from 3-8 membered heterocyclyl, wherein the above-mentioned group is optionally further substituted with 0-3 groups selected from: halogen; in certain embodiments, R₁₂ is selected from C₁₋₈ alkoxy, wherein the above-mentioned group is optionally further substituted with 0-3 groups selected from: halogen; and the definitions of other groups are consistent with those of embodiment four.

In certain embodiments, R₁₂ is selected from

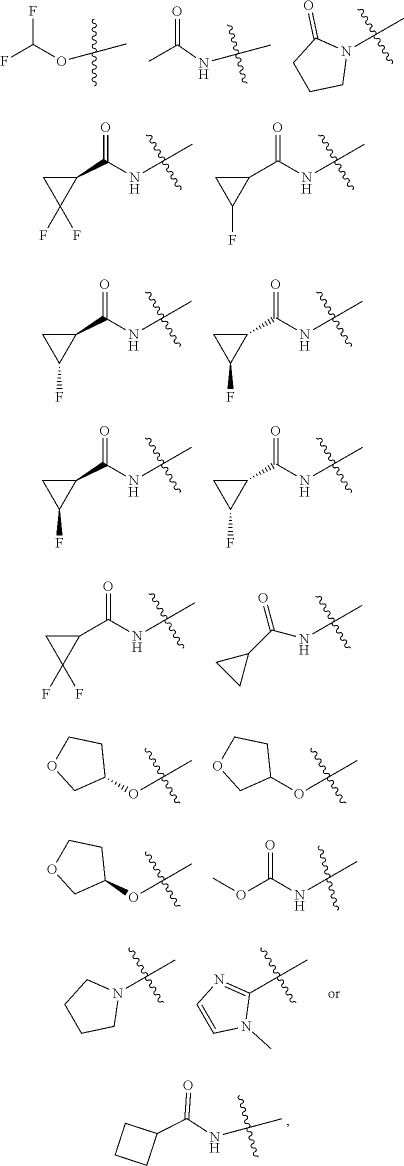

and the definitions of other groups are consistent with those of embodiment four.
In certain embodiments, R¹² is selected from

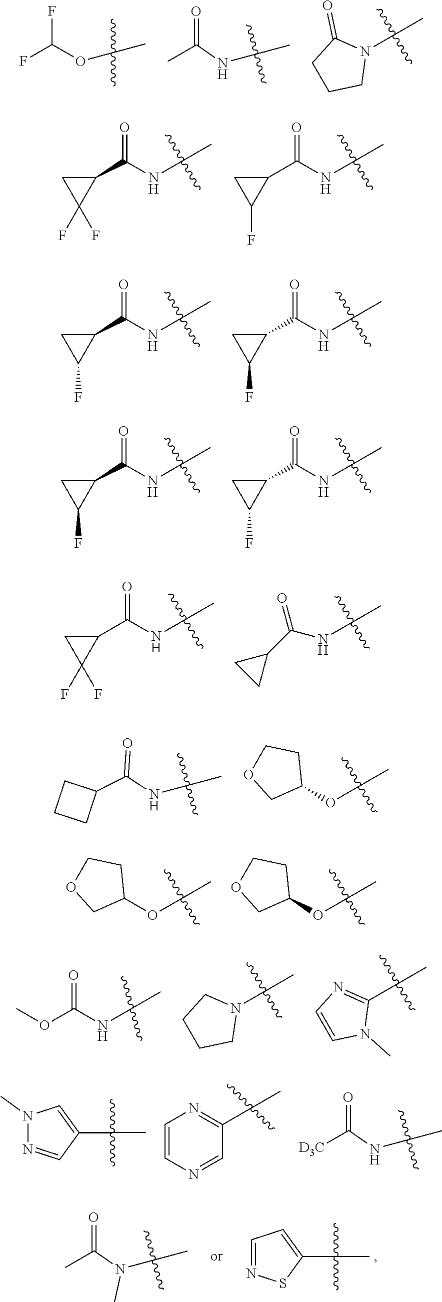

and the definitions of other groups are consistent with those of embodiment four.
In the compound represented by general formula (VII), the isomer thereof or the pharmaceutically acceptable salt thereof according to embodiment four, R¹² is selected from

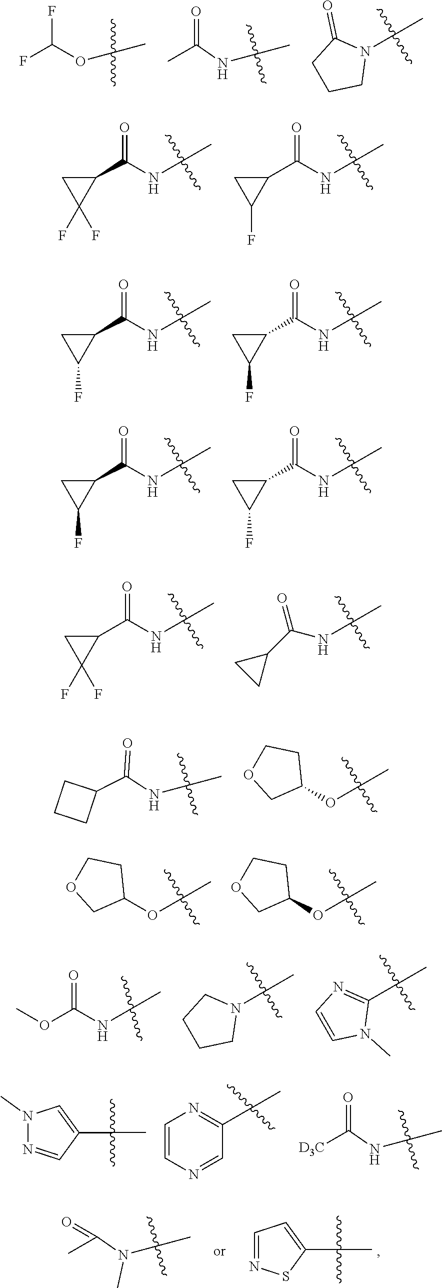

or R¹² is selected from

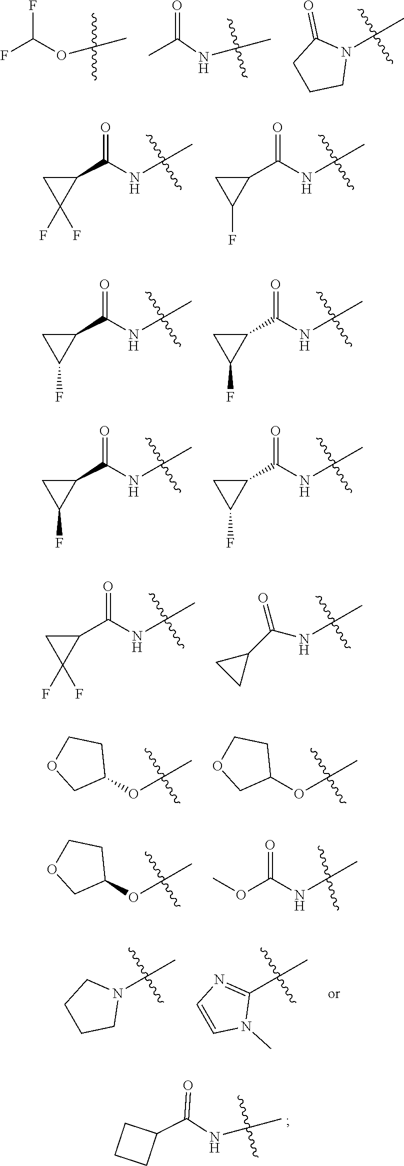

q is selected from 0, 1, 2, 3, 4 and 5;
Y₃ is selected from piperazinylene;
R₂₉ is selected from difluoromethyl;
and R₃₀ is selected from H.

In certain embodiments, q is selected from 0, and the definitions of other groups are consistent with those of embodiment four.

In certain embodiments, R¹² is selected from

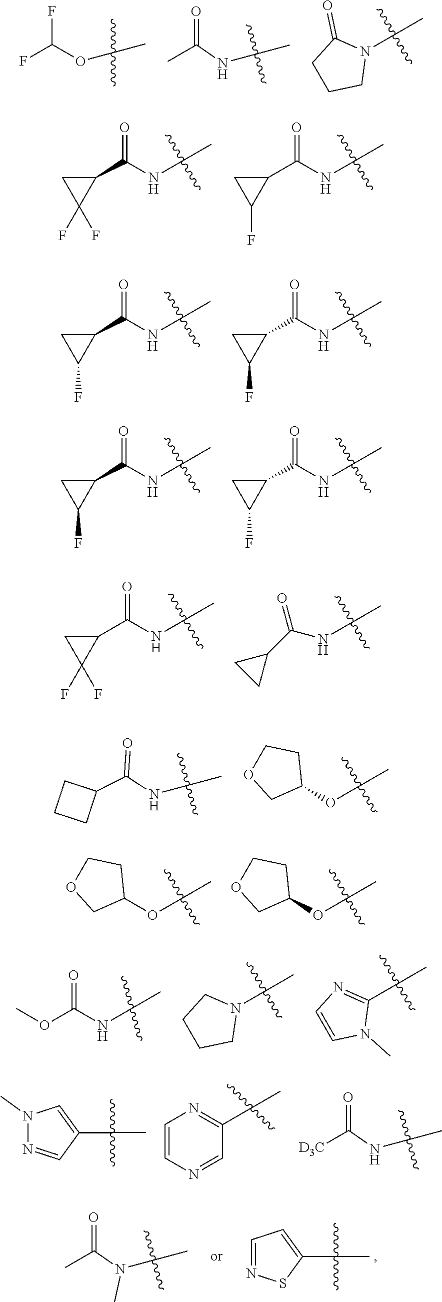

R¹² is selected from

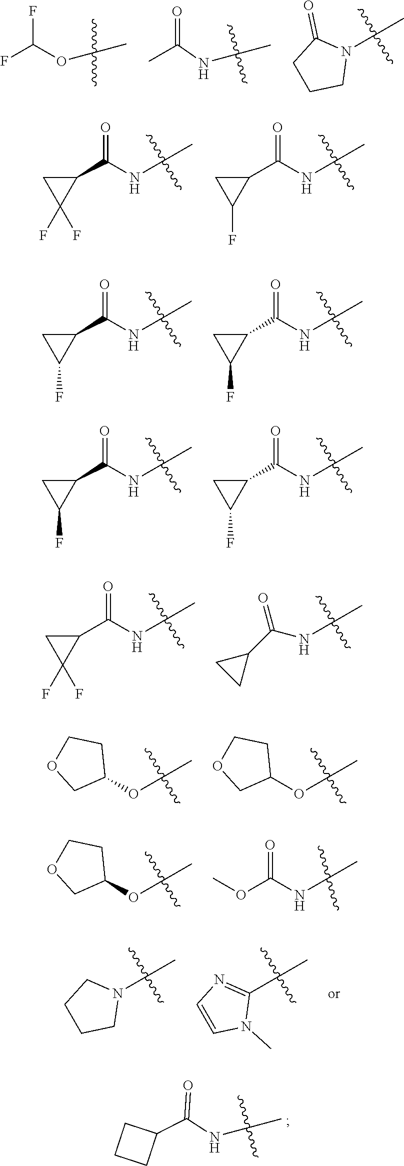

q is selected from 0;
Y₃ is selected from piperazinylene;
R₂₉ is selected from difluoromethyl;
and R₃₀ is selected from H.

In certain embodiments, R¹² is selected from

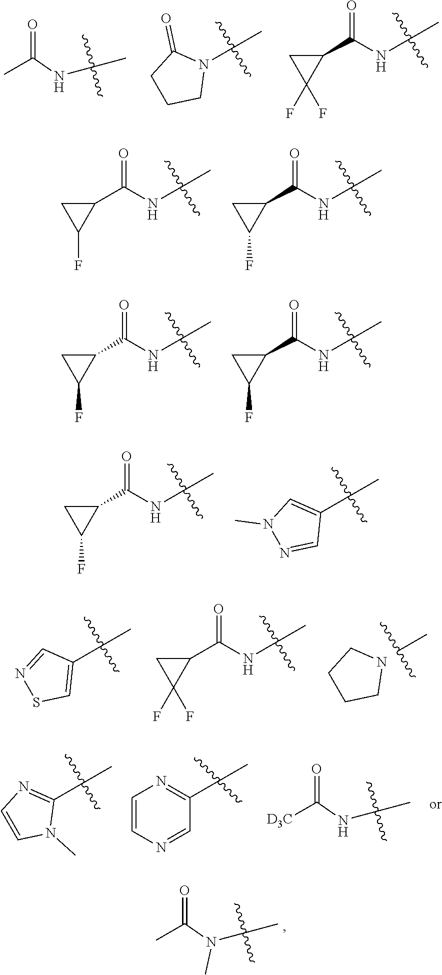

R¹² is selected from

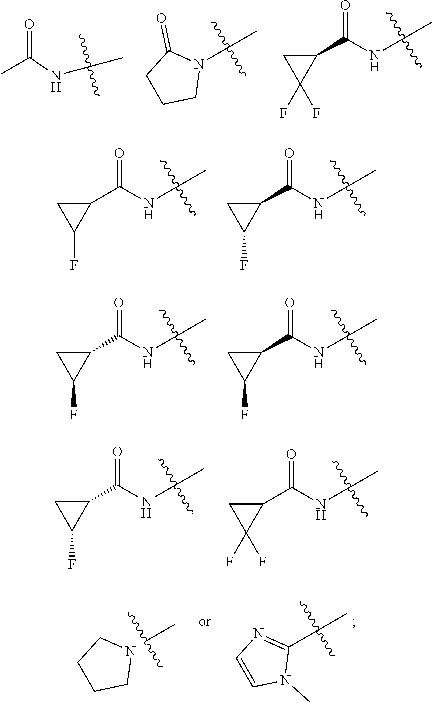

q is selected from 0;
Y₃ is selected from piperazinylene;
R₂₉ is selected from difluoromethyl;
and R₃₀ is selected from H. In certain embodiments, R¹² is selected from

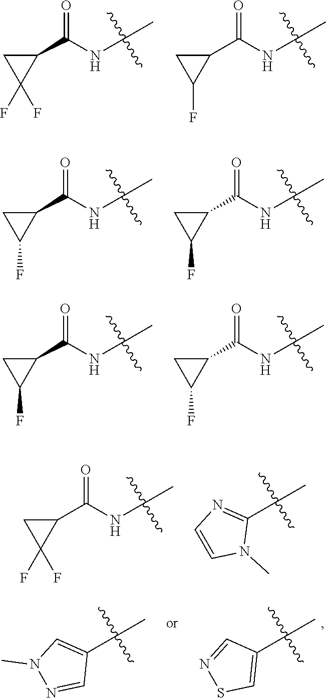

or
R¹² is selected from

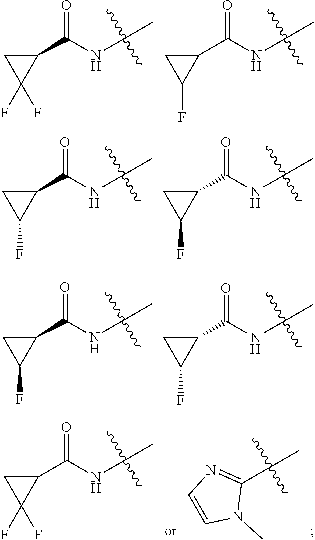

q is selected from 0;
Y₃ is selected from piperazinylene;
R₂₉ is selected from difluoromethyl;
and R₃₀ is selected from H.

Embodiment five of the present disclosure relates to a compound represented by general formula (I-a), an isomer thereof or a pharmaceutically acceptable salt thereof,

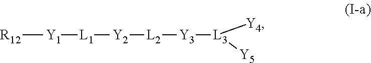

wherein:
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally further substituted with 0-5 R^{y1}; in certain embodiments, Y₁ is selected from C₅₋₁₅ aryl, 5-15 membered heteroaryl and 3-12 membered heterocyclyl, wherein the aryl, heteroaryl and heterocyclyl are optionally further substituted with 0-5 R^{y1}; in certain embodiments, Y₁ is selected from 5-15 membered heteroaryl and 3-12 membered heterocyclyl, wherein the heteroaryl and heterocyclyl are optionally further substituted with 0-5 R^{y1}; in certain embodiments, Y₁ is selected from 5-15 membered heteroaryl, wherein the heteroaryl is optionally further substituted with 0-5 R^{y1}; in certain embodiments, Y₁ is selected from phenyl,

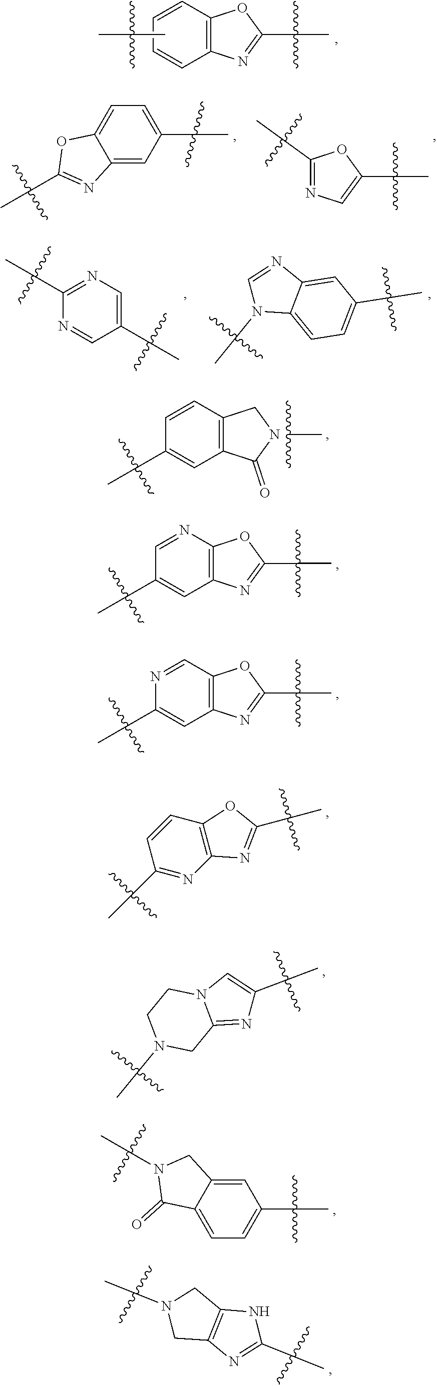

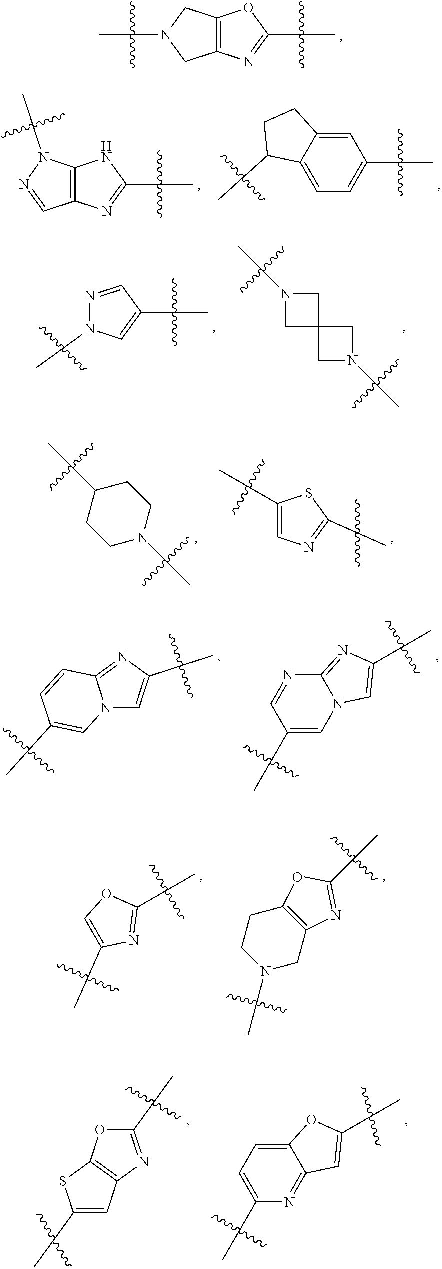

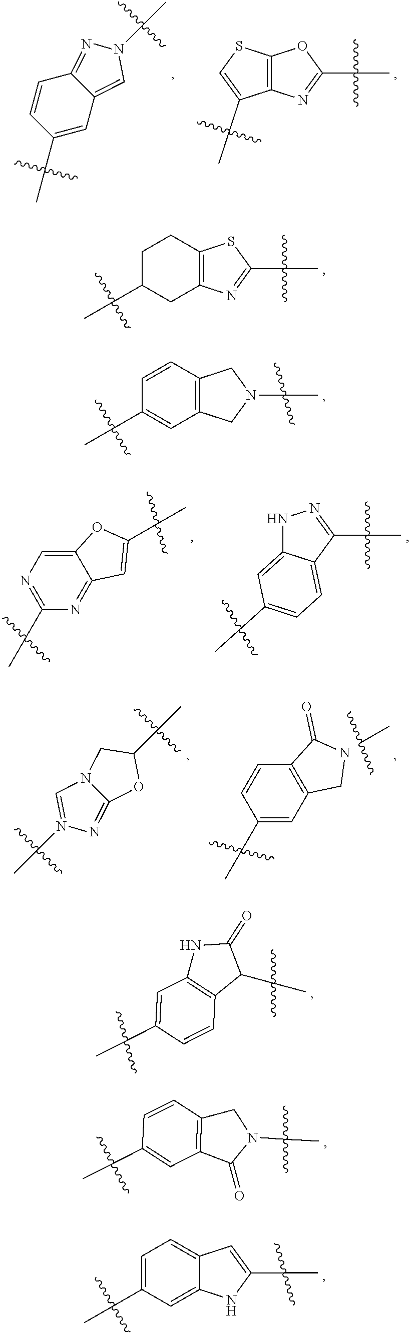

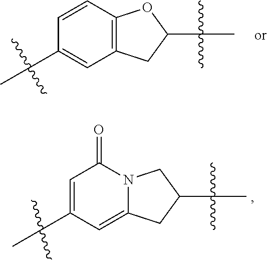 [SMILES: *c1ccc2c(c1)CC(*)O2 ; *c1cc2n(c(=O)c1)CC(*)C2]

wherein the above-mentioned groups are optionally substituted with 0-5 R^{y1};
in certain embodiments, Y is selected from phenyl and

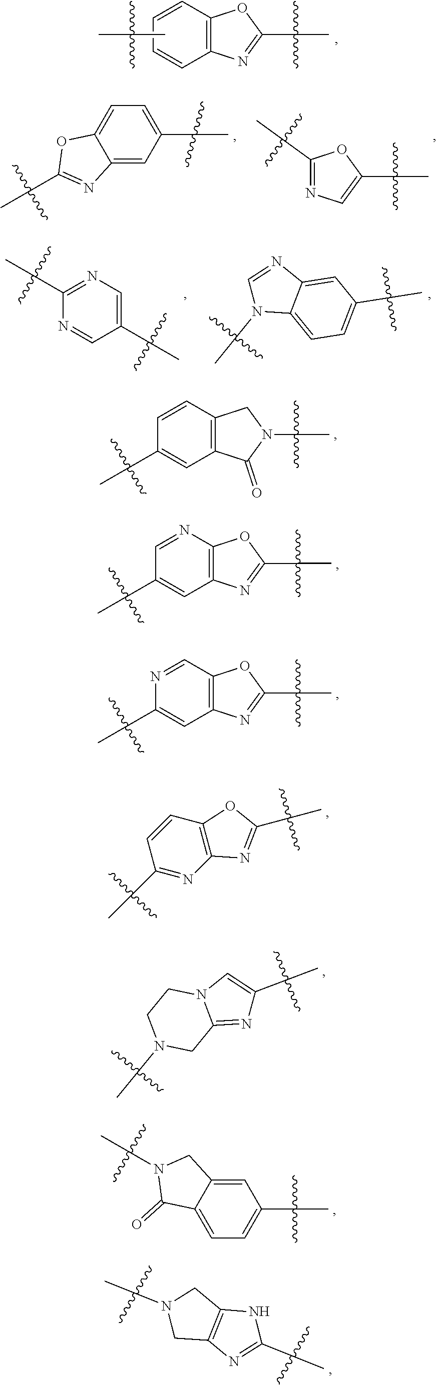

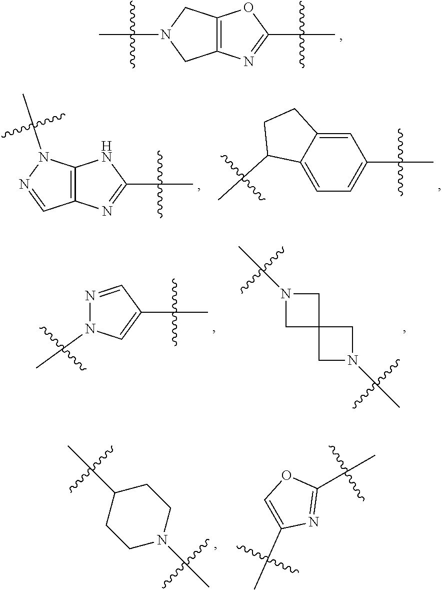

and optionally substituted with 0-5 R^{y1}; in certain embodiments, Y₁ is selected from phenyl or,

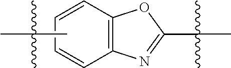

and optionally substituted with 0-5 R^{y1}; in certain embodiments, Y₁ is selected from phenyl or

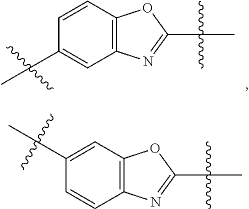 [SMILES: *c1ccc2oc(*)nc2c1 ; *c1ccc2nc(*)oc2c1]

and optionally substituted with 0-5 R^{y1}; in certain embodiments, Y₁ is selected from phenyl or

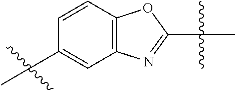 [SMILES: *c1ccc2oc(*)nc2c1]

and optionally substituted with 0-5 R^{y1}; in certain embodiments, Y₁ is selected from

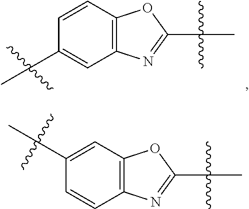 [SMILES: *c1ccc2oc(*)nc2c1 ; *c1ccc2nc(*)oc2c1]

and optionally substituted with 0-5 R^{y1}; in certain embodiments, Y₁ is selected from

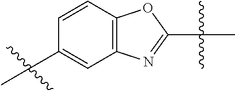 [SMILES: *c1ccc2oc(*)nc2c1]

and optionally substituted with 0-5 R^{y1}; in certain embodiments, Y₁ is selected from

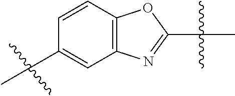 [SMILES: *c1ccc2oc(*)nc2c1]

and optionally substituted with 0-5 R^{y1};
Y₂ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y2}; in certain embodiments, Y₂ is selected from C₅₋₁₅ aryl, 5-15 membered heteroaryl and 3-12 membered heterocyclyl, wherein the aryl, heteroaryl and heterocyclyl are optionally substituted with 0-5 R^{y2}; in certain embodiments, Y₂ is selected from 5-15 membered heteroaryl and 3-12 membered heterocyclyl, wherein the heteroaryl and heterocyclyl are optionally substituted with 0-5 R^{y2}; in certain embodiments, Y₂ is selected from 5-15 membered heteroaryl, wherein the heteroaryl is optionally substituted with 0-5 R^{y2}; in certain embodiments, Y₂ is selected from phenyl,

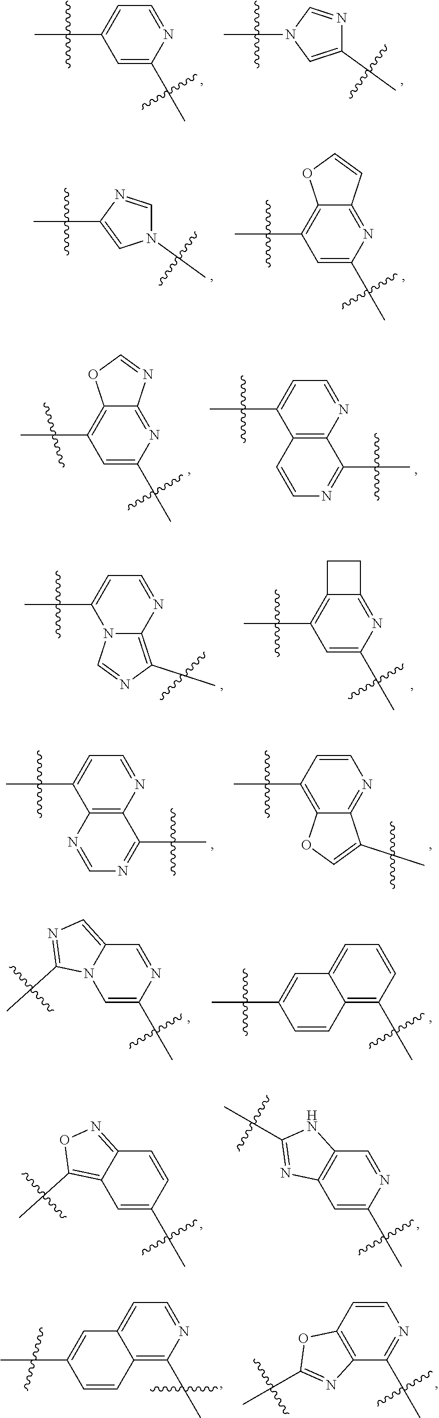

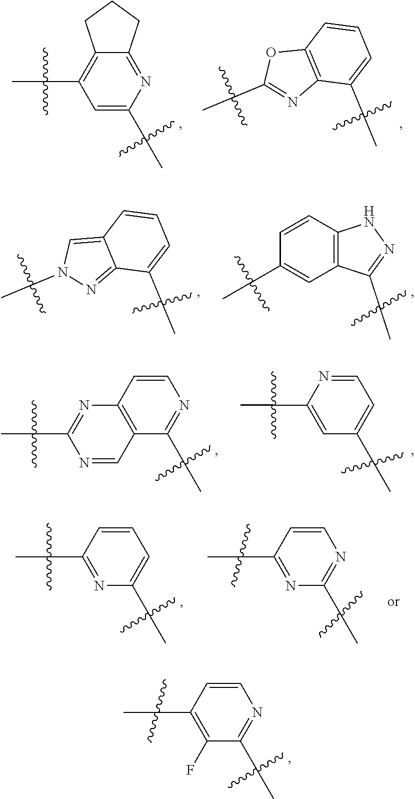

wherein the above-mentioned groups are optionally substituted with 0-5 R^{y2}; in certain embodiments, Y₂ is selected from phenyl,

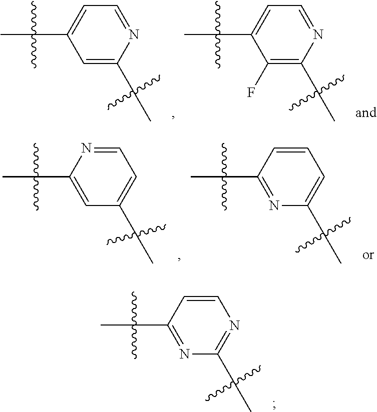 [SMILES: *c1ccnc(*)c1 ; *c1ccnc(*)c1F ; *c1ccnc(*)c1 ; *c1cccc(*)n1 ; *c1ccnc(*)n1]

in certain embodiments, Y₂ is selected from phenyl,

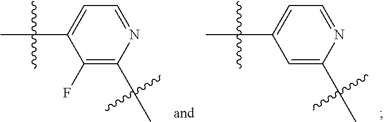 [SMILES: *c1ccnc(*)c1F ; *c1ccnc(*)c1]

in certain embodiments, Y₂ is selected from phenyl and

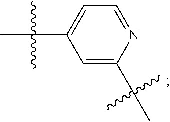 [SMILES: *c1ccnc(*)c1]

in certain embodiments, Y₂ is selected from

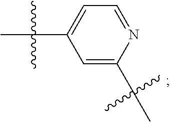 [SMILES: *c1ccnc(*)c1]

Y₃ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y3}; in certain embodiments, Y₃ is selected from C₅₋₁₅ aryl, 5-15 membered heteroaryl and 3-12 membered heterocyclyl, wherein the aryl, heteroaryl and heterocyclyl are optionally substituted with 0-5 R^{y3}; in certain embodiments, Y₃ is selected from 5-15 membered heteroaryl and 3-12 membered heterocyclyl, wherein the heteroaryl and heterocyclyl are optionally substituted with 0-5 R^{y3}; in certain embodiments, Y₃ is selected from 3-12 membered heterocyclyl, wherein the heteroaryl and heterocyclyl are optionally substituted with 0-5 R^{y3}; in certain embodiments, Y₃ is selected from

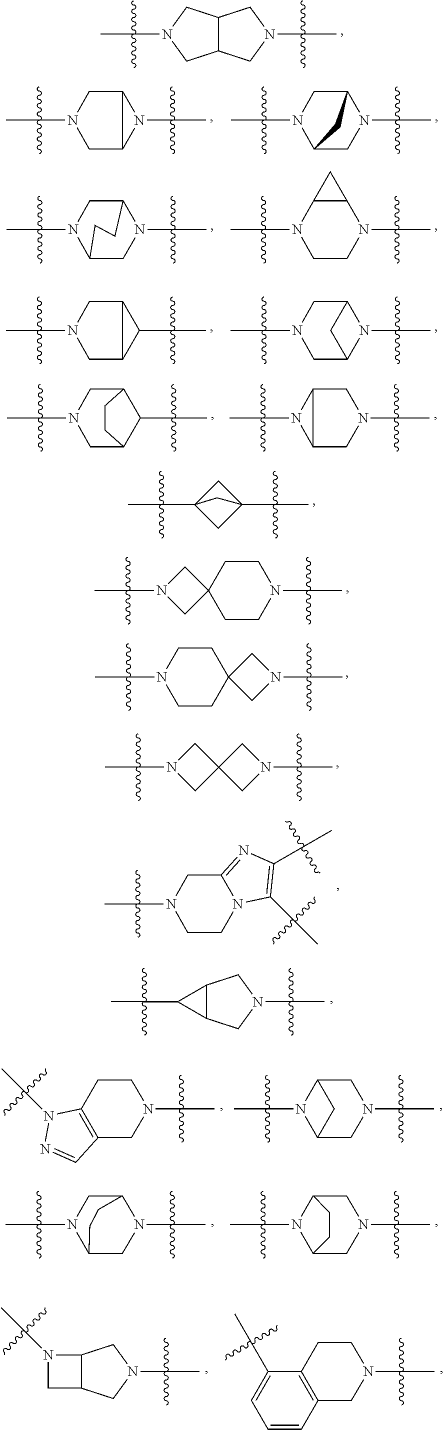

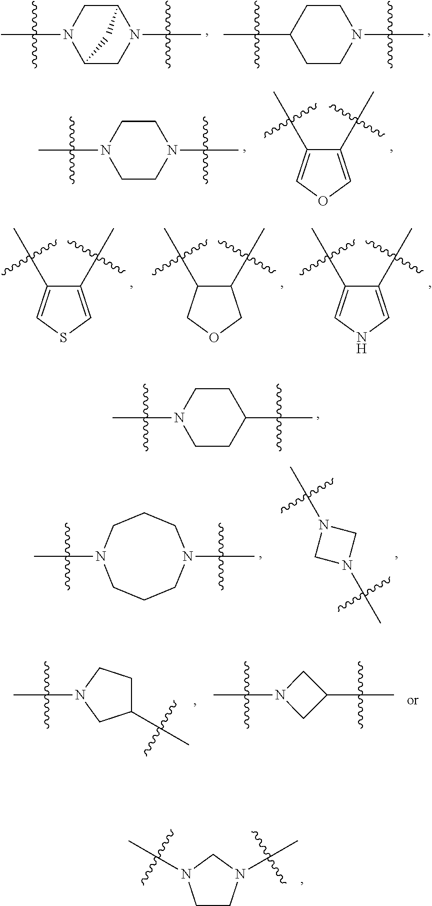

wherein the above-mentioned groups are optionally substituted with 0-5 R^{y3}; in certain embodiments, Y₃ is selected from

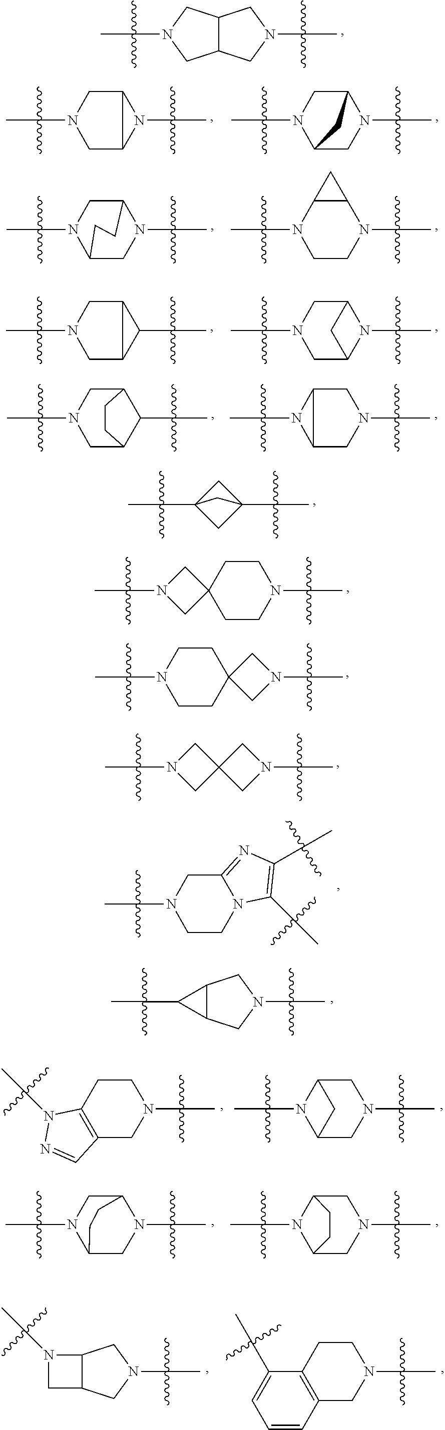

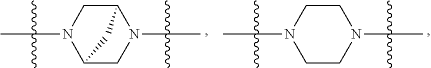 [SMILES: *N1C[C@@H]2C[C@H]1CN2* ; *N1CCN(*)CC1]

wherein the above-mentioned groups are optionally substituted with 0-5 R^{y3}; in certain embodiments, Y₃ is selected from

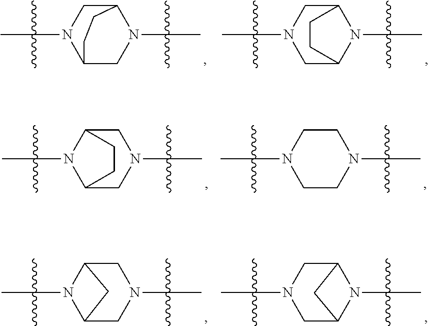 [SMILES: *N1CC2CCC1CN2* ; *N1CC2CCC(C1)N2* ; *N1CC2CCC(C1)N2* ; *N1CCN(*)CC1 ; *N1CC2CC(C1)N2* ; *N1CC2CC(C1)N2*]

wherein the above-mentioned groups are optionally substituted with 0-5 R^{y3}; in certain embodiments, Y₃ is selected from

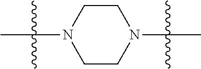 [SMILES: *N1CCN(*)CC1]

and optionally substituted with 0-3 R^{y3}; in certain embodiments, Y₃ is selected from

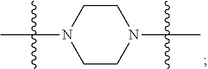 [SMILES: *N1CCN(*)CC1]

in certain embodiments, Y₃ is selected from

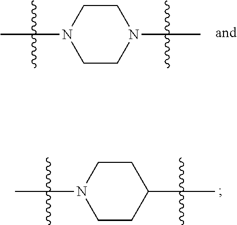 [SMILES: *N1CCN(*)CC1 ; *C1CCN(*)CC1]

in certain embodiments, Y₃ is selected from

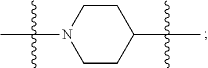 [SMILES: *C1CCN(*)CC1]

Y₄ is selected from C₃₋₁₂ carbocyclyl, C₅₋₁₅ aryl, 5-15 membered heteroaryl, 3-12 membered heterocyclyl, C₂₋₈ alkenyl and C₂₋₈ alkynyl, wherein the carbocyclyl, aryl, heteroaryl, heterocyclyl, alkenyl and alkynyl are optionally substituted with 0-5 R^{y4}; in certain embodiments, Y₄ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y4}; in certain embodiments, Y₄ is selected from C₅₋₁₅ aryl, 5-15 membered heteroaryl and 3-12 membered heterocyclyl, wherein the aryl, heteroaryl and heterocyclyl are optionally substituted with 0-5 R^{y4}; in certain embodiments, Y₄ is selected from C₅₋₁₅ aryl and 5-15 membered heteroaryl, wherein the aryl and heteroaryl are optionally substituted with 0-5 R^{y4}; in certain embodiments, Y₄ is selected from C₅₋₁₅ aryl, wherein the aryl is optionally substituted with 0-5 R^{y4}; in certain embodiments, Y₄ is selected from C₅₋₁₅ aryl; in certain embodiments, Y₄ is selected from phenyl, ethynyl, cyclopentyl, and

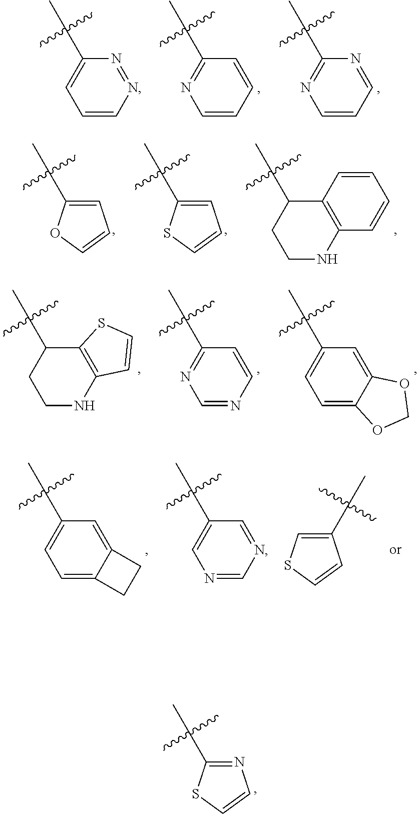

wherein the above-mentioned groups are optionally substituted with 0-5 R^{y4}; in certain embodiments, Y₄ is selected from phenyl optionally substituted with 0-5 R^{y4}; in certain embodiments, Y₄ is selected from phenyl;
Y₅ is selected from C₃₋₁₂ carbocyclyl, C₅₋₁₅ aryl, 5-15 membered heteroaryl, 3-12 membered heterocyclyl, —C(O)NHOCH₃, —C(O)NHCN, —P(O)(OCH₃)₂ and —C(O)OCH₂CH₃, wherein the carbocyclyl, aryl, heteroaryl and heterocyclyl are optionally substituted with 0-5 R^{y5}; in certain embodiments, Y₅ is selected from C₅₋₁₅ aryl, 5-15 membered heteroaryl and 3-12 membered heterocyclyl, wherein the aryl, heteroaryl and heterocyclyl are optionally substituted with 0-5 R^{y5}; in certain embodiments, Y₅ is selected from 5-15 membered heteroaryl and 3-12 membered heterocyclyl, wherein the heteroaryl and heterocyclyl are optionally substituted with 0-5 R^{y5}; in certain embodiments, Y₅ is selected from 5-15 membered heteroaryl, wherein the heteroaryl is optionally substituted with 0-5 R^{y5}; in certain embodiments, Y₅ is selected from 5-15 membered heteroaryl, wherein the heteroaryl is optionally substituted with 0-3 R^{y5}; in certain embodiments, Y₅ is selected from

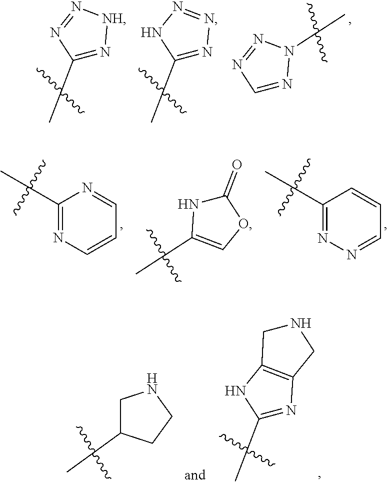

wherein the above-mentioned groups are optionally substituted with 0-3 R^{y5}; in certain embodiments, Y₅ is selected from

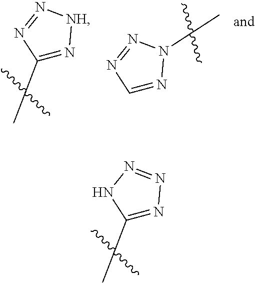

and optionally substituted with 0-3 R^{y5}; in certain embodiments, Y₅ is selected from

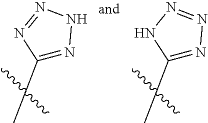 [SMILES: *c1nn[nH]n1 ; *c1nnn[nH]1]

and optionally substituted with 0-3 R^{y5}; in certain embodiments, Y₅ is selected from

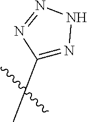 [SMILES: *c1nn[nH]n1]

and optionally substituted with 0-3 R^{y5}; in certain embodiments, Y₅ is selected from

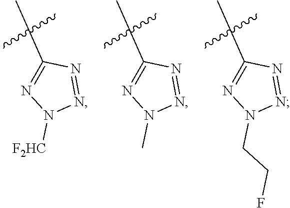

in certain embodiments, Y₅ is selected from

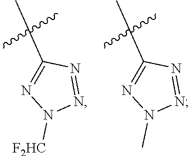

certain embodiments, Y₅ is selected from

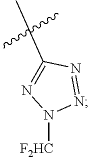 [SMILES: *c1nnn(C(F)F)n1]

in certain embodiments, Y₅ is selected from

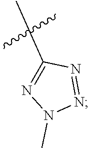 [SMILES: *c1nnn(C)n1]

in certain embodiments, Y₅ is selected from

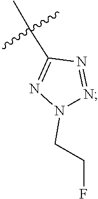 [SMILES: *c1nnn(CCF)n1]

in certain embodiments, Y₅ is selected from tetrazolyl and

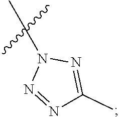

in certain embodiments, Y₅ is selected from

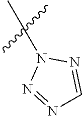

and optionally substituted with 0-3 R^{y5}; in certain embodiments, Y₅ is selected from

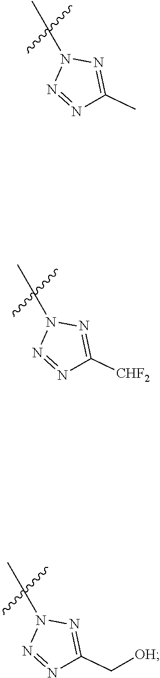

R^{y1}, R^{y2}, R^{y3}, R^{y4} and R^{y5} are each independently selected from halogen, ═O, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; in certain embodiments, R^{y1}, R^{y2}, R^{y3}, R^{y4} and R^{y5} are each independently selected from halogen, ═O, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ hydroxylalkyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; in certain embodiments, each R^{y1} is independently selected from halogen, ═O, hydroxyl, cyano, C₁₋₈ alkyl and C₁₋₈ alkoxy, wherein the alkyl and alkoxy are optionally further substituted with 0-5 groups selected from: halogen; in certain embodiments, each R^{y1} is independently selected from halogen, ═O, hydroxyl and C₁₋₈ alkyl, wherein the alkyl is optionally further substituted with 0-5 groups selected from: halogen; in certain embodiments, each R^{y1} is independently selected from halogen, ═O and C₁₋₈ alkyl, wherein the alkyl is optionally further substituted with 0-5 groups selected from: halogen; in certain embodiments, each R^{y1} is independently selected from halogen and C₁₋₈ alkyl, wherein the alkyl is optionally further substituted with 0-5 groups selected from: halogen; in certain embodiments, each R^{y1} is independently selected from methyl, ═O, hydroxyl and F; in certain embodiments, each R^{y1} is independently selected from methyl, ═O and F; in certain embodiments, each R^{y1} is independently selected from methyl; in certain embodiments, each R^{y1} is independently selected from F; in certain embodiments, each R^{y2} is independently selected from halogen, ═O, hydroxyl, cyano, C₁₋₈ alkyl and C₁₋₈ alkoxy, wherein the alkyl and alkoxy are optionally further substituted with 0-5 groups selected from: halogen; in certain embodiments, each R^{y2} is independently selected from halogen, cyano, C₁₋₈ alkyl and C₁₋₈ alkoxy, wherein the alkyl and alkoxy are optionally further substituted with 0-5 groups selected from: halogen; in certain embodiments, each R^{y2} is independently selected from halogen, methyl, trifluoromethyl, difluoromethyl, cyano and methoxy; in certain embodiments, each R^{y2} is independently selected from halogen; in certain embodiments, each R^{y2} is independently selected from F; in certain embodiments, each R^{y3} is independently selected from halogen, ═O, hydroxyl, cyano and C₁₋₈ alkyl, wherein the alkyl is optionally further substituted with 0-5 groups selected from: halogen; in certain embodiments, each R^{y3} is independently selected from halogen, hydroxyl and C₁₋₈ alkyl, wherein the alkyl is optionally further substituted with 0-5 groups selected from: halogen; in certain embodiments, each R^{y3} is independently selected from halogen, ═O, hydroxyl, methyl, difluoromethyl and trifluoromethyl; in certain embodiments, each R^{y3} is independently selected from F, ═O and hydroxyl; in certain embodiments, each R^{y4} is independently selected from halogen, C₁₋₈ alkyl, C₃₋₈ cycloalkyl and C₁₋₈ alkoxy, wherein the alkyl, cycloalkyl and alkoxy are optionally further substituted with 0-5 groups selected from: halogen; in certain embodiments, each R^{y4} is independently selected from F and cyclopropyl; in certain embodiments, each R^{y4} is independently selected from F; in certain embodiments, each R^{y5} is independently selected from C₁₋₈ alkyl and C₁₋₈ alkoxy, wherein the alkyl and alkoxy are optionally further substituted with 0-5 groups selected from: halogen and hydroxyl; in certain embodiments, each R^{y5} is independently selected from methyl, fluoroethyl and difluoromethyl; in certain embodiments, each R^{y5} is independently selected from methyl and difluoromethyl; in certain embodiments, each R^{y5} is independently selected from methyl; in certain embodiments, each R^{y5} is independently selected from difluoromethyl;
or R^{y3} and the atom on Y₃ ring form C₃₋₈ cycloalkyl, and in certain embodiments, form cyclopropyl; or
R^{y1} and R^{y2} together with L₁ form 4-8 membered heterocyclyl, C₄₋₈ carbocyclyl or 5-8 membered heteroaryl;
R₁₂ is selected from H, cyano, C₁₋₈ alkoxy, C₃₋₈ cycloalkyl,

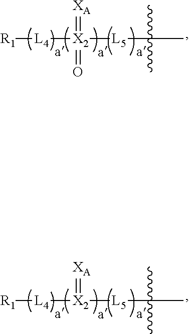

3-8 membered heterocyclyl, C₅₋₁₀ aryl or 5-10 membered heteroaryl, wherein the cycloalkyl, heterocyclyl, aryl or heteroaryl is optionally further substituted with 0-5 of the following groups: halogen, ═O, acetamido, hydroxyl, cyano, sulfhydryl, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; in certain embodiments, R₁₂ is selected from H, cyano, C₁₋₈ alkoxy, C₃₋₈ cycloalkyl,

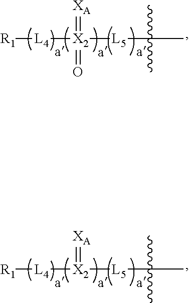

3-8 membered heterocyclyl, C₅₋₁₀ aryl or 5-10 membered heteroaryl, wherein the cycloalkyl, heterocyclyl, aryl or heteroaryl is optionally further substituted with 0-5 of the following groups: halogen, ═O, acetamido, hydroxyl, cyano, sulfhydryl, nitro, C₁₋₈ alkyl, C₁₋₈ alkoxy, C₁₋₈ haloalkyl, C₂-8 alkenyl, C₂₋₈ alkynyl, C₃₋₈ cycloalkyl, 3-8 membered heterocyclyl, C₅₋₁₀ aryl or 5-10 membered heteroaryl; in certain embodiments, R₁₂ is selected from cyano,

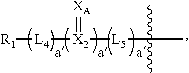

3-8 membered heterocyclyl, C₅₋₁₀ aryl or 5-10 membered heteroaryl, wherein the heterocyclyl, aryl or heteroaryl is optionally further substituted with 0-5 of the following groups: halogen, ═O, acetamido and hydroxyl; in certain embodiments, R₁₂ is selected from

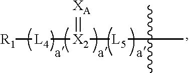

3-8 membered heterocyclyl and 5-10 membered heteroaryl, wherein the heterocyclyl or heteroaryl is optionally further substituted with 0-5 of the following groups: halogen, ═O, acetamido and hydroxyl; in certain embodiments, R₁₂ is selected from

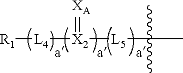

and 5-10 membered heteroaryl, wherein the heteroaryl is optionally further substituted with 0-5 of the following groups: halogen, ═O, acetamido and hydroxyl; in certain embodiments, R₁₂ is selected from

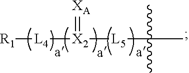

in certain embodiments, R₁₂ is selected from:

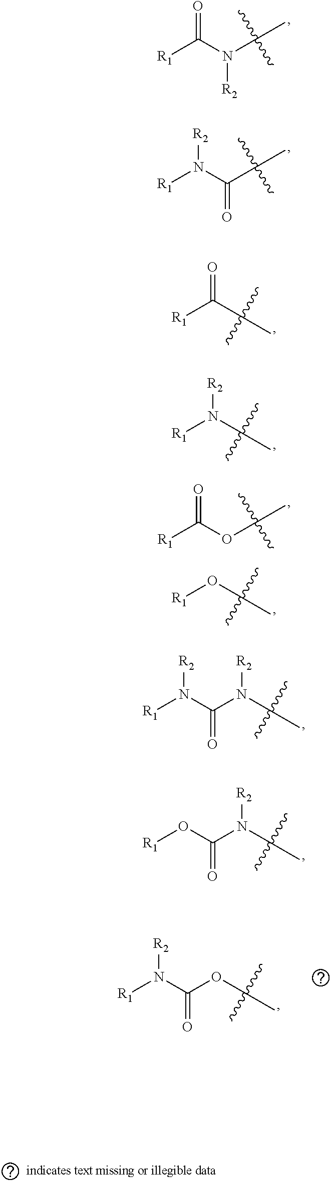

3-8 membered heterocyclyl, C₅₋₁₀ aryl or 5-10 membered heteroaryl, wherein the heterocyclyl, aryl, and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
in certain embodiments, R₁₂ is selected from

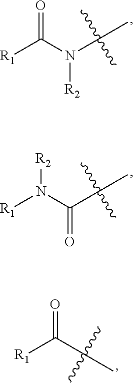

3-8 membered heterocyclyl or 5-6 membered heteroaryl, wherein the heterocyclyl and heteroaryl are optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl; in certain embodiments, R₁₂ is selected from 5-10 membered heteroaryl, wherein the heteroaryl is optionally further substituted with 0-5 of the following groups: halogen, ═O, acetamido and hydroxyl; in certain embodiments, R₁₂ is selected from acetyl, acetamido, difluoromethyloxy, cyano, pyridyl, thiazolyl, pyrrolidinyl, pyrazinyl,

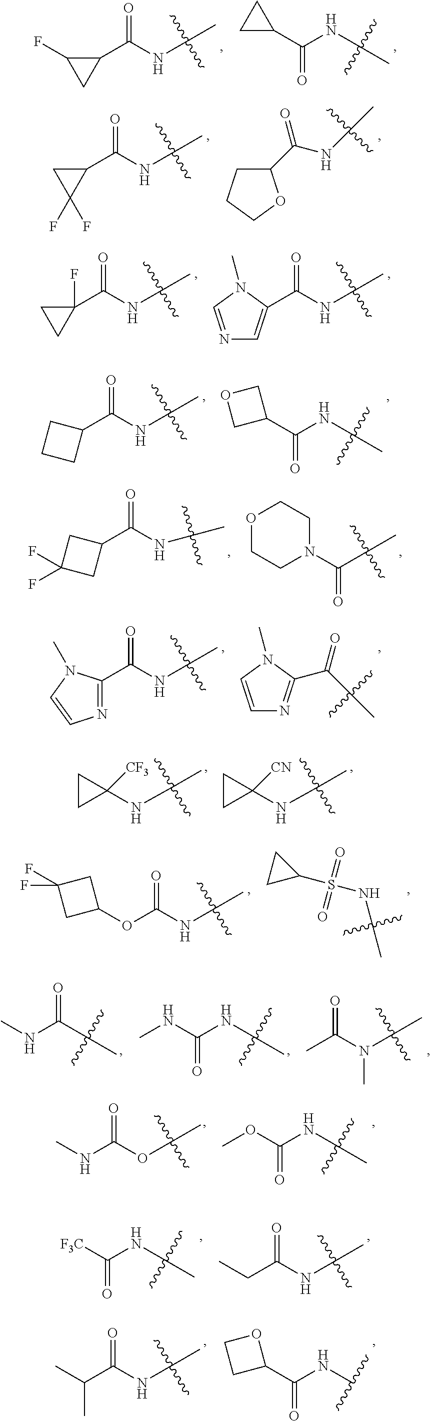

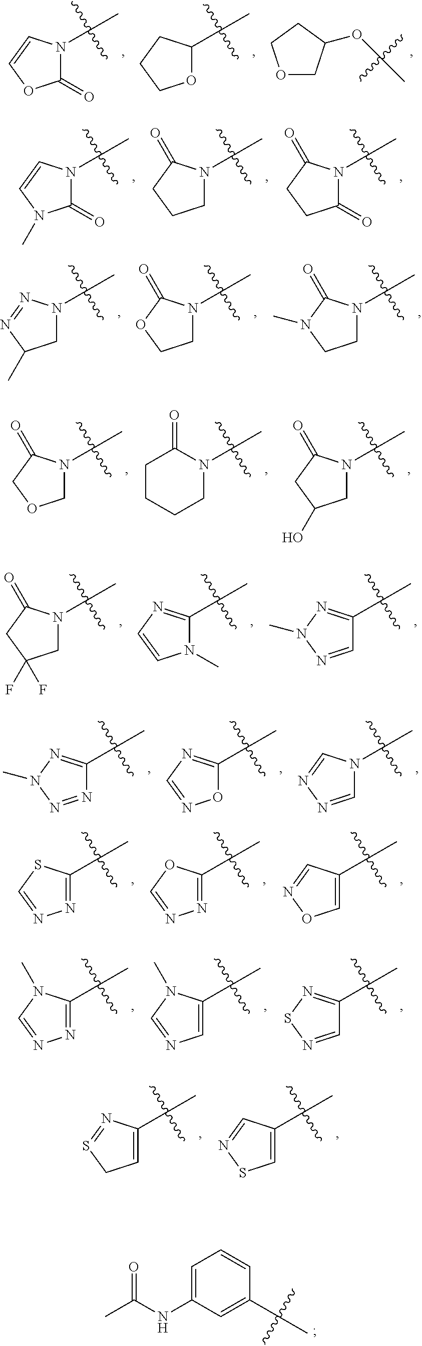

in certain embodiments, R₁₂ is selected from

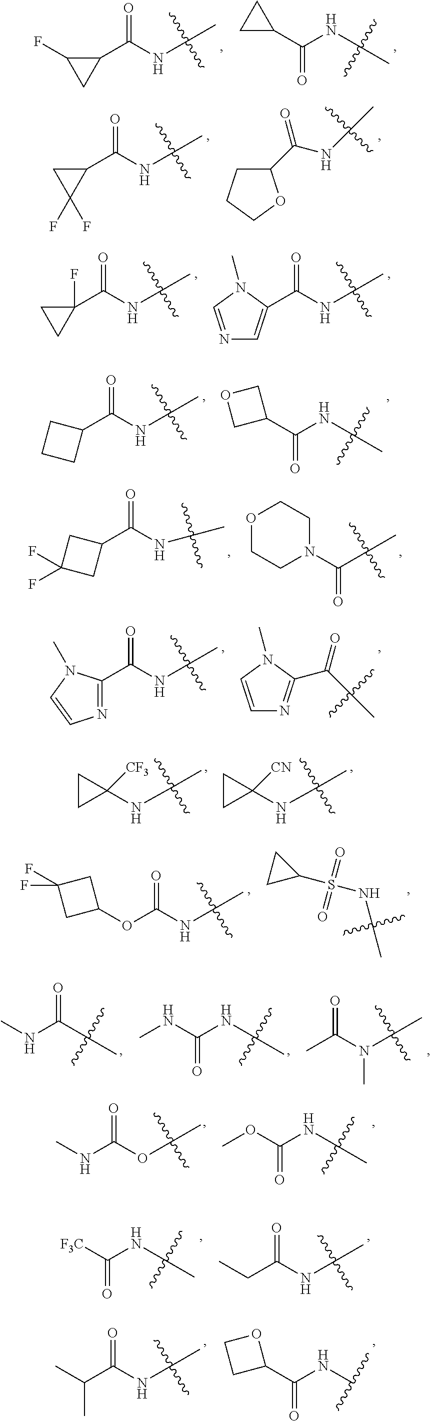

in certain embodiments, R₁₂ is selected from

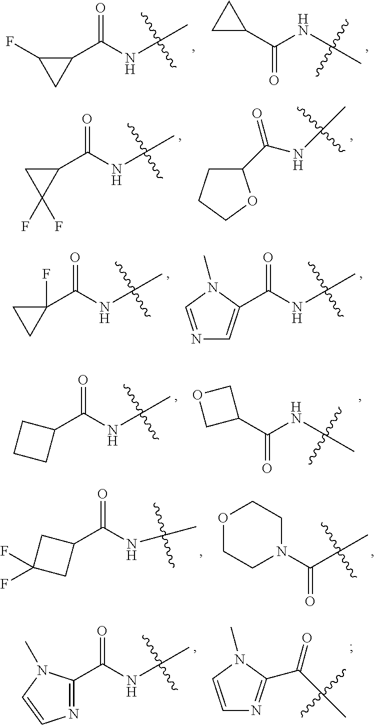

in certain embodiments, R₁₂ is selected from

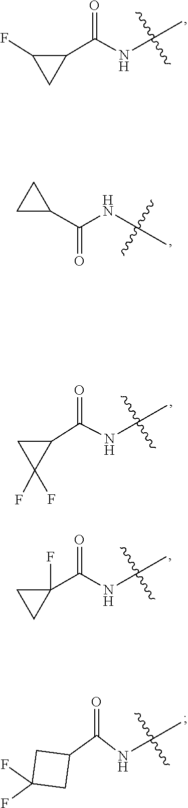 [SMILES: *NC(=O)C1CC1F ; *NC(=O)C1CC1 ; *NC(=O)C1CC1(F)F ; *NC(=O)C1(F)CC1 ; *NC(=O)C1CC(F)(F)C1]

in certain embodiments, R₁₂ is selected from

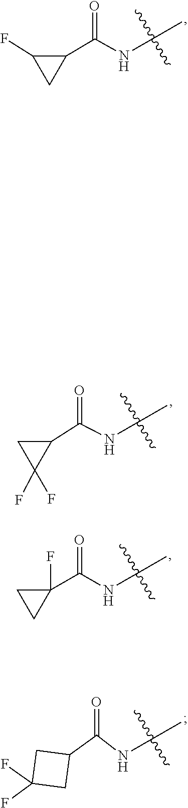 [SMILES: *NC(=O)C1CC1F ; *NC(=O)C1CC1(F)F ; *NC(=O)C1(F)CC1 ; *NC(=O)C1CC(F)(F)C1]

in certain embodiments, R₁₂ is selected from:

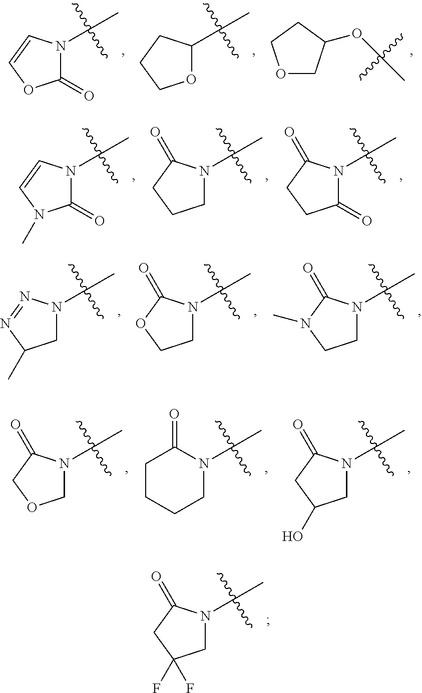

in certain embodiments, R₁₂ is selected from:

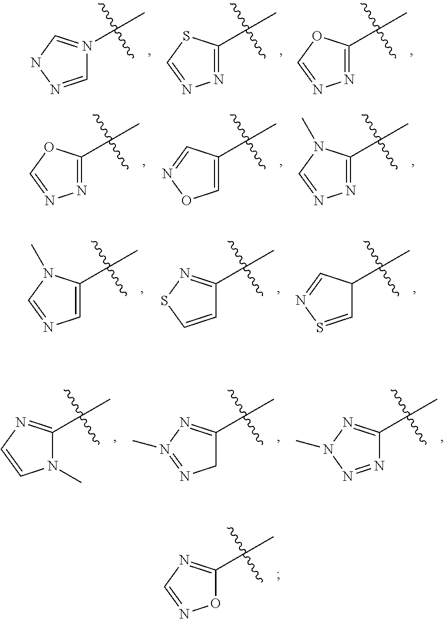

in certain embodiments, R₁₂ is selected from:

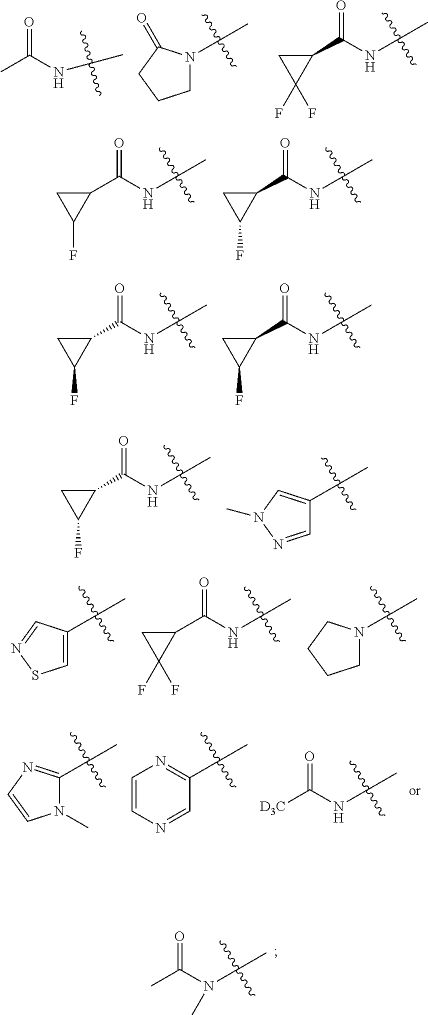

in certain embodiments, R₁₂ is selected from:

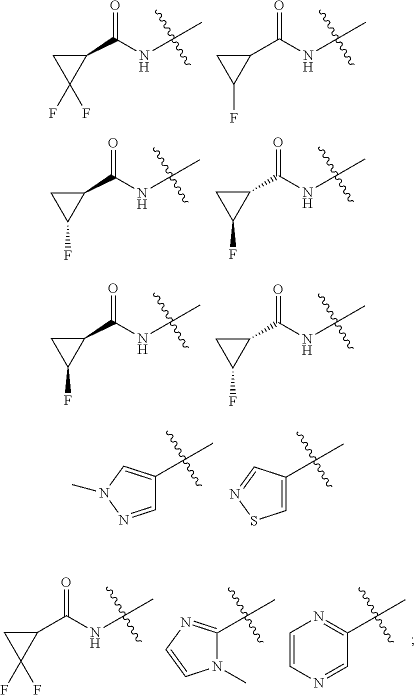

in certain embodiments, R₁₂ is selected from acetamido,

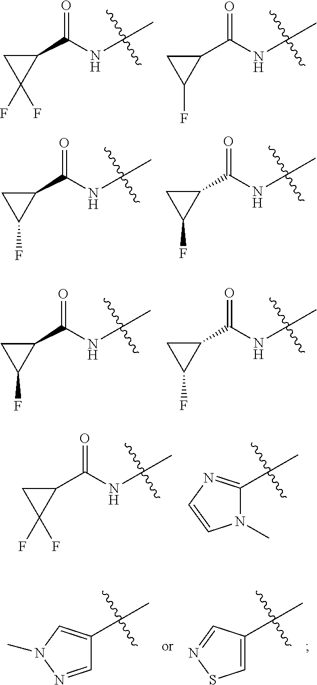

in certain embodiments, R₁₂ is selected from acetamido,

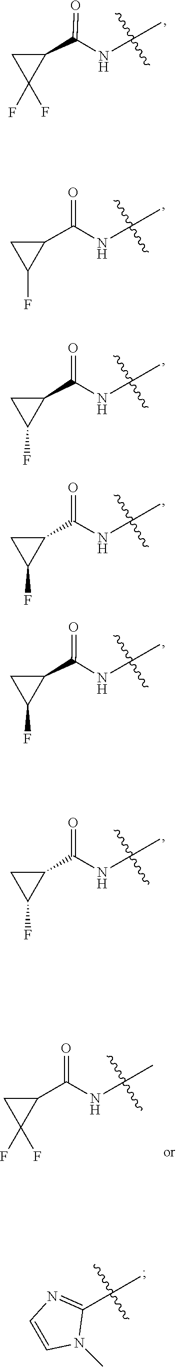

in certain embodiments, R₁₂ is selected from acetamido; in certain embodiments, R₁₂ is selected from

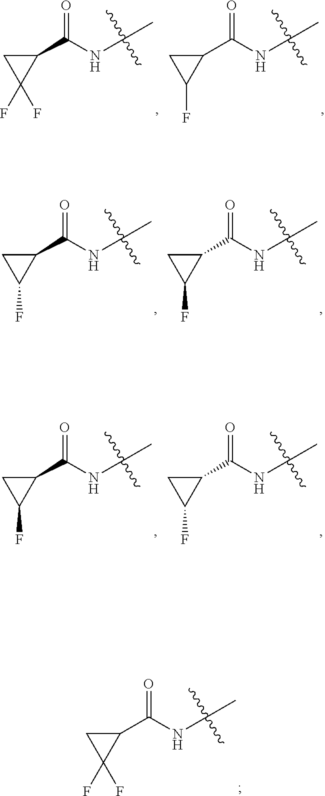

in certain embodiments, R₁₂ is selected from

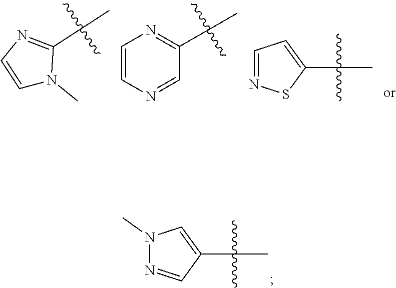

in certain embodiments, R₁₂ is selected from

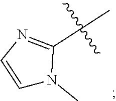 [SMILES: *c1nccn1C]

X₁ is selected from P or S; in certain embodiments, X₁ is selected from P;
X₂ is selected from C, PR₁ₐ or S; in certain embodiments, X₂ is selected from C; L₄ and L₅ are each independently selected from: a bond, NR₂, CR₃R₄, O, or S; in certain embodiments, L₄ and L₅ are each independently selected from: a bond, NR₂, CR₃R₄ and O; in certain embodiments, each L₄ is independently selected from a bond, NR₂ and O; in certain embodiments, each L₄ is independently selected from a bond, NR₂ and CR₃R₄; in certain embodiments, each L₄ is independently selected from CR₃R₄; in certain embodiments, each L₄ is independently selected from O; in certain embodiments, each L₄ is independently selected from a bond; in certain embodiments, each L₄ is independently selected from NR₂; in certain embodiments, each L₅ is independently selected from: a bond, NR₂, CR₃R₄ and O; in certain embodiments, each L₅ is independently selected from: NR₂ and O; in certain embodiments, each L₅ is independently selected from: a bond and NR₂; in certain embodiments, each L₅ is independently selected from: a bond; in certain embodiments, each L₅ is independently selected from NR₂; in certain embodiments, each L₅ is independently selected from CR₃R₄;
X_{A} is selected from C, NR₂ₐ, O and S; in certain embodiments, X_{A} is selected from O;
a is 0 or 1; in certain embodiments, a is 1; in certain embodiments, a is 0;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, C₁₋₈ alkyl, cyano, ═O, C₁₋₈ alkoxy or C₁₋₈ haloalkyl; in certain embodiments, R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: hydroxyl, halogen, C₁₋₈ alkyl, cyano, ═O, C₁₋₈ alkoxy or C₁₋₈ haloalkyl; in certain embodiments, R₁ is selected from H, halogen, cyano, C₁₋₈ alkyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, cycloalkyl, alkoxy, haloalkyl, heterocyclyl, aryl or heteroaryl is optionally further substituted with 0-5 groups selected from: deuterium, hydroxyl, halogen, C₁₋₈ alkyl, cyano, ═O, C₁₋₈ alkoxy or C₁₋₈ haloalkyl; in certain embodiments, R₁ is selected from C₁₋₈ alkyl, C₃₋₈ cycloalkyl, 3-8 membered heterocyclyl and 5-10 membered heteroaryl, wherein the alkyl, cycloalkyl, heterocyclyl or heteroaryl is optionally further substituted with 0-5 groups selected from: deuterium, hydroxyl, halogen, C₁₋₈ alkyl, cyano, ═O and C₁₋₈ haloalkyl; in certain embodiments, R₁ is selected from methyl, cycloalkyl, cyclobutyl, oxacyclopentyl and

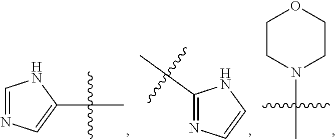 [SMILES: *c1cnc[nH]1 ; *c1ncc[nH]1 ; *N1CCOCC1]

wherein these groups are optionally substituted with 0-3 groups selected from: deuterium, hydroxyl, halogen, C₁₋₈ alkyl, cyano, ═O and C₁₋₈ haloalkyl; in certain embodiments, R₁ is selected from methyl and cycloalkyl, wherein these groups are optionally substituted with 0-3 groups selected from: deuterium, hydroxyl, halogen, C₁₋₈ alkyl, cyano, ═O and C₁₋₈ haloalkyl; R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: halogen, C₁₋₈ alkyl, C₁₋₈ alkoxy or C₁₋₈ haloalkyl; in certain embodiments, R₁ₐ, R₂, R₂ₐ, R₃ and R₄ are each independently selected from: H, halogen, carbonyl C₁₋₈ alkyl, C₃₋₈ cycloalkyl, C₁₋₈ alkoxy and C₁₋₈ haloalkyl, wherein the alkyl, cycloalkyl, alkoxy and haloalkyl are optionally further substituted with 0-3 groups selected from: halogen, C₁₋₈ alkyl, C₁₋₈ alkoxy or C₁₋₈ haloalkyl; in certain embodiments, R₁ₐ, R₂, R₂ₐ, R₃ and R₄ are each independently selected from: H, halogen, C₁₋₈ alkyl, C₃₋₈ cycloalkyl, C₁₋₈ alkoxy and C₁₋₈ haloalkyl, wherein the alkyl, cycloalkyl, alkoxy and haloalkyl are optionally further substituted with 0-3 groups selected from: halogen; in certain embodiments, each R₁ₐ is independently selected from: H and C₁₋₈ alkoxy; in certain embodiments, R₂ and R₂ₐ are each independently selected from: H and C₁₋₈ alkyl, wherein each alkyl is optionally further substituted with 0-3 groups selected from: halogen; in certain embodiments, R₂ and R₂ₐ are each independently selected from: H; in certain embodiments, R₃ and R₄ are each independently selected from: H, halogen, C₁₋₈ alkyl and C₁₋₈ haloalkyl; in certain embodiments, R₃ and R₄ are each independently selected from: H;
L₁ is selected from a bond, —(CR₆)ₜ—NR₅—(CR₇)_{t′}—, C₂₋₈ alkynyl, C₂₋₈ alkenyl,

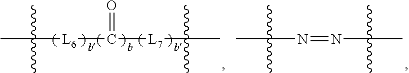

3-8 membered heterocyclyl, C₃₋₈ cycloalkyl, C₅₋₁₀ aryl or 5-10 membered heteroaryl, wherein the alkynyl, alkenyl, heterocyclyl, cycloalkyl, aryl or heteroaryl is optionally substituted with 0-5 groups selected from: hydroxyl, halogen, C₁₋₈ alkyl or C₁₋₈ haloalkyl; in certain embodiments, L₁ is selected from a bond, —(CR₆)ₜ—NR₅—(CR₇)_{t′}—, C₂₋₈ alkynyl, C₂₋₈ alkenyl,

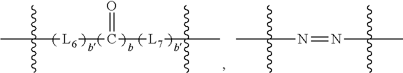

and C₃₋₈ cycloalkyl, wherein the alkynyl, alkenyl and cycloalkyl are optionally substituted with 0-3 groups selected from: hydroxyl, halogen, C₁₋₈ alkyl or C₁₋₈ haloalkyl; in certain embodiments, L₁ is selected from a bond and —(CR₆)ₜ—NR₅—(CR₇)_{t′}—; in certain embodiments, L₁ is selected from a bond;
t and t′ are selected from 0, 1, 2 and 3; in certain embodiments, t and t′ are selected from 0 and 1;
L₂ is selected from a bond,

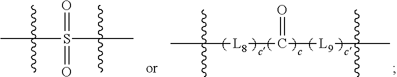

in certain embodiments, L₂ is selected from

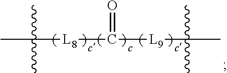

in certain embodiments, L₂ is selected from carbonyl; in certain embodiments, L₂ is selected from a bond;
L₆, L₇, L₈ and L₉ are each independently selected from a bond, O, S, NR₅ or CR₆R₇; in certain embodiments, L₆, L₇, L₈ and L₉ are each independently selected from a bond and NR₅; in certain embodiments, L₆, L₇, L₈ and L₉ are each independently selected from a bond;
b and c are each independently selected from 0 or 1; in certain embodiments, b and c are each independently selected from 0;
b′ and c′ are each independently selected from 0, 1, 2, 3, 4, 5 or 6; in certain embodiments, b′ and c′ are each independently selected from 0 and 1; in certain embodiments, b′ and c′ are each independently selected from 0;
R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl; in certain embodiments, R₅, R₆ and R₇ are each independently selected from: H, halogen, C₁₋₈ alkyl and C₁₋₈ haloalkyl; in certain embodiments, each R₅ is independently selected from: H and C₁₋₈ alkyl, and R₆ and R₇ are each independently selected from: H, halogen, C₁₋₈ alkyl and C₁₋₈ haloalkyl; in certain embodiments, each R₅ is independently selected from: H, and R₆ and R₇ are each independently selected from: H, halogen and C₁₋₈ alkyl; in certain embodiments, each R₅ is independently selected from: H and C₁₋₈ alkyl, and R₆ and R₇ are each independently selected from: H and C₁₋₈ alkyl; in certain embodiments, each R₅ is independently selected from: H and C₁₋₈ alkyl, and R₆ and R₇ are each independently selected from: H;
L₃ is selected from a bond,

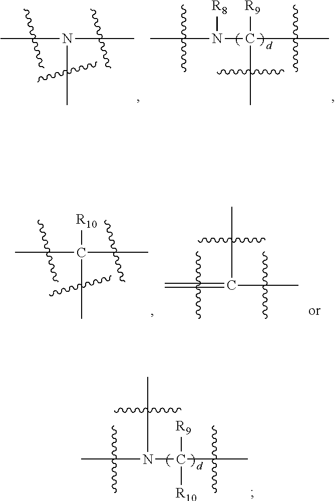

in certain embodiments, L₃ is selected from

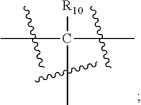

in certain embodiments, L₃ is selected from

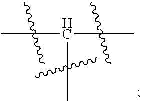 [SMILES: *C(*)*]

d is an integer selected from 1-6; in certain embodiments, d is selected from 1;
R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl; in certain embodiments, R₈, R₉ and R₁₀ are each independently selected from H, C₁₋₈ alkyl; in certain embodiments, R₈, R₉ and R₁₀ are each independently selected from H;
provided that:
L₁, L₂ and L₃ are not simultaneously a bond;
when Y₁ is selected from

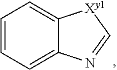

X^{y1} is O or S, L₁ is a bond, L₂ is carbonyl, L₃ is

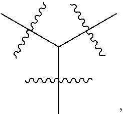 [SMILES: *C(*)*]

Y₂ is selected from

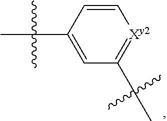

X^{y2} is C or N, Y₃ is selected from

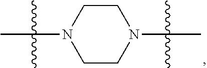 [SMILES: *N1CCN(*)CC1]

Y₄ is selected from a benzene ring, Y₁ is substituted with 0 R^{y1}, and R₁₂ is selected from acetamido, halogen, methyl, methoxy, trifluoromethyl, trifluoromethyloxy, hydroxyl, methanesulfonylamido, methylamino or N,N-dimethylamino, Y₅ is not selected from the following groups:

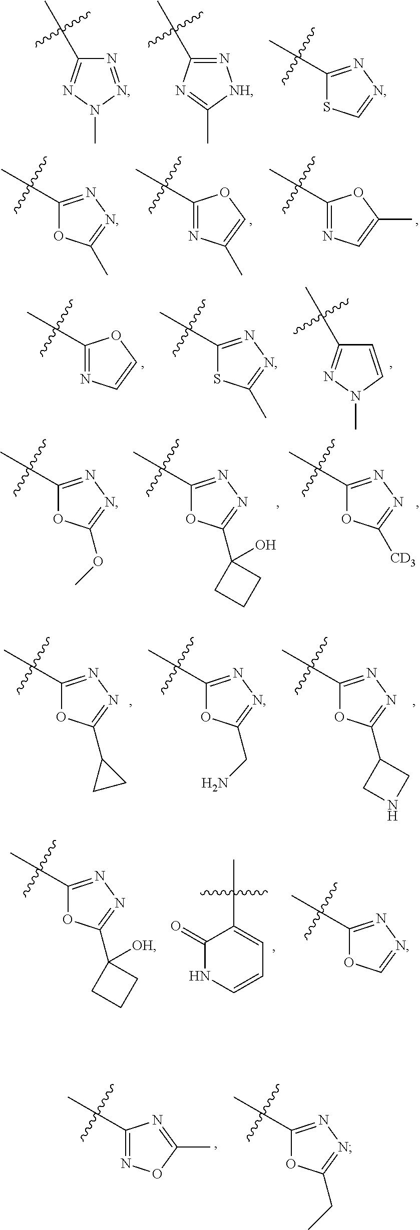

when R₁₂—Y₁ is selected from

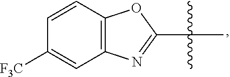 [SMILES: *c1nc2cc(C(F)(F)F)ccc2o1]

L₁ is a bond, L₂ is carbonyl, L₃ is

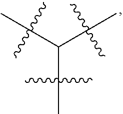 [SMILES: *C(*)*]

Y₃ is selected from

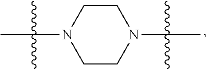 [SMILES: *N1CCN(*)CC1]

Y₄ is selected from a benzene ring and Y₅ is selected from

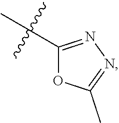 [SMILES: *c1nnc(C)o1]

Y₂ is not selected from the following groups:

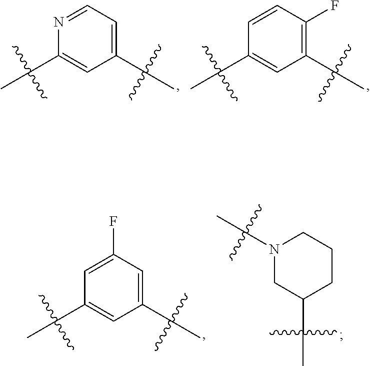 [SMILES: *c1ccnc(*)c1 ; *c1ccc(F)c(*)c1 ; *c1cc(*)cc(F)c1 ; *C1CCCN(*)C1]

when Y₅ is selected from acetamido, L₃ is a bond;
and when Y₅ is —C(O)OCH₂CH₃, it does not have the following structure:

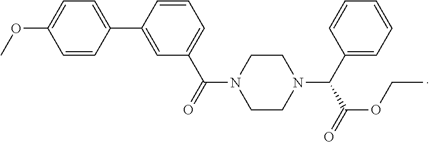 [SMILES: CCOC(=O)[C@@H](c1ccccc1)N1CCN(C(=O)c2cccc(-c3ccc(OC)cc3)c2)CC1]

In certain embodiments, embodiment five of the present disclosure relates to a compound represented by general formula (I-a), an isomer thereof or a pharmaceutically acceptable salt thereof:

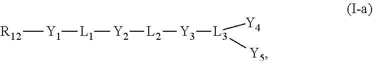

wherein:
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally further substituted with 0-5 R^{y1};
Y₂ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y2};
Y₃ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y3};
Y₄ is selected from C₃₋₁₂ carbocyclyl, C₅₋₁₅ aryl, 5-15 membered heteroaryl, 3-12 membered heterocyclyl, C₂₋₈ alkenyl and C₂₋₈ alkynyl, wherein the carbocyclyl, aryl, heteroaryl, heterocyclyl, alkenyl and alkynyl are optionally substituted with 0-5 R^{y4};
Y₅ is selected from C₃₋₁₂ carbocyclyl, C₅₋₁₅ aryl, 5-15 membered heteroaryl, 3-12 membered heterocyclyl, —C(O)NHOCH₃, —C(O)NHCN, —P(O)(OCH₃)₂ and —C(O)OCH₂CH₃, wherein the carbocyclyl, aryl, heteroaryl and heterocyclyl are optionally substituted with 0-5 R^{y5};
R^{y1}, R^{y2}, R^{y3}, R^{y4} and R^{y5} are each independently selected from halogen, ═O, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ hydroxylalkyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; or
R^{y3} and the atom on Y₃ ring form C₃₋₈ cycloalkyl; or
R^{y1} and R^{y2} together with L₁ form 4-8 membered heterocyclyl, C₄₋₈ carbocyclyl or 5-8 membered heteroaryl;
R₁₂ is selected from H, cyano, C₁₋₈ alkoxy, C₃₋₈ cycloalkyl,

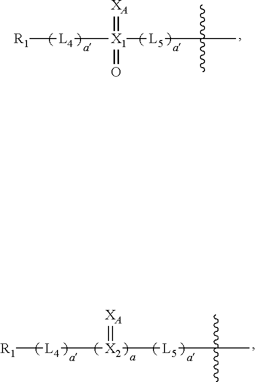

3-8 membered heterocyclyl, C₅₋₁₀ aryl or 5-10 membered heteroaryl, wherein the cycloalkyl, heterocyclyl, aryl or heteroaryl is optionally further substituted with 0-5 of the following groups: halogen, ═O, acetamido, hydroxyl, cyano, sulfhydryl, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₁ is selected from P or S;
X₂ is selected from C, PR₁ₐ or S;
L₄ and L₅ are each independently selected from: a bond, NR₂, CR₃R₄, O, or S;
X_{A} is selected from C, NR₂ₐ, O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, C₁₋₈ alkyl, cyano, ═O, C₁₋₈ alkoxy or C₁₋₈ haloalkyl;
R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: halogen, C₁₋₈ alkyl, C₁₋₈ alkoxy or C₁₋₈ haloalkyl;
L₁ is selected from a bond, —(CR₆)ₜ—NR₅—(CR₇)_{t′}—, C₂₋₈ alkynyl, C₂₋₈ alkenyl,

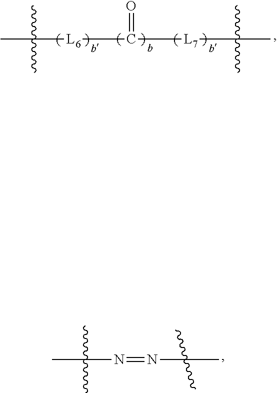

3-8 membered heterocyclyl, C₃₋₈ cycloalkyl, C₅₋₁₀ aryl or 5-10 membered heteroaryl, wherein the alkynyl, alkenyl, heterocyclyl, cycloalkyl, aryl or heteroaryl is optionally substituted with 0-5 groups selected from: hydroxyl, halogen, C₁₋₈ alkyl or C₁₋₈ haloalkyl;
t and t′ are selected from 0, 1, 2 and 3;
L₂ is selected from a bond,

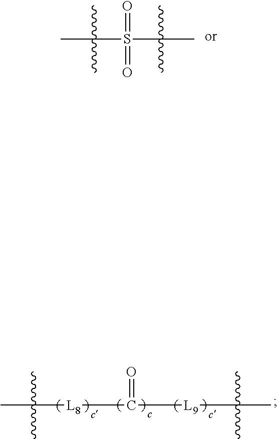

L₆, L₇, L₈ and L₉ are each independently selected from a bond, O, S, NR₅ or CR₆R₇; b and c are each independently selected from 0 or 1; b′ and c′ are each independently selected from 0, 1, 2, 3, 4, 5 or 6; R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl;
L₃ is selected from a bond,

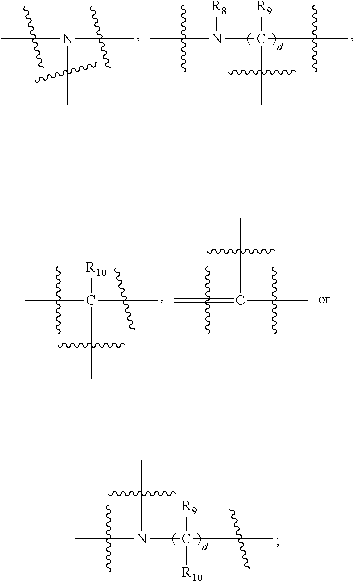

d is an integer selected from 1-6; and R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl; provided that:
L₁, L₂ and L₃ are not simultaneously a bond;
when Y₁ is selected from

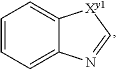

X^{y1} is O or S, L₁ is a bond, L₂ is carbonyl, L₃ is

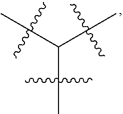 [SMILES: *C(*)*]

Y₂ is selected from

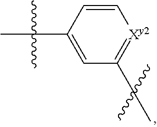

X^{y2} is C or N, Y₃ is selected from

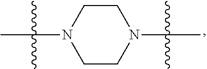 [SMILES: *N1CCN(*)CC1]

Y₄ is selected from a benzene ring, Y₁ is substituted with 0 R^{y1}, and R₁₂ is selected from acetamido, halogen, methyl, methoxy, trifluoromethyl, trifluoromethyloxy, hydroxyl, methanesulfonylamido, methylamino or N,N-dimethylamino, Y₅ is not selected from the following groups:

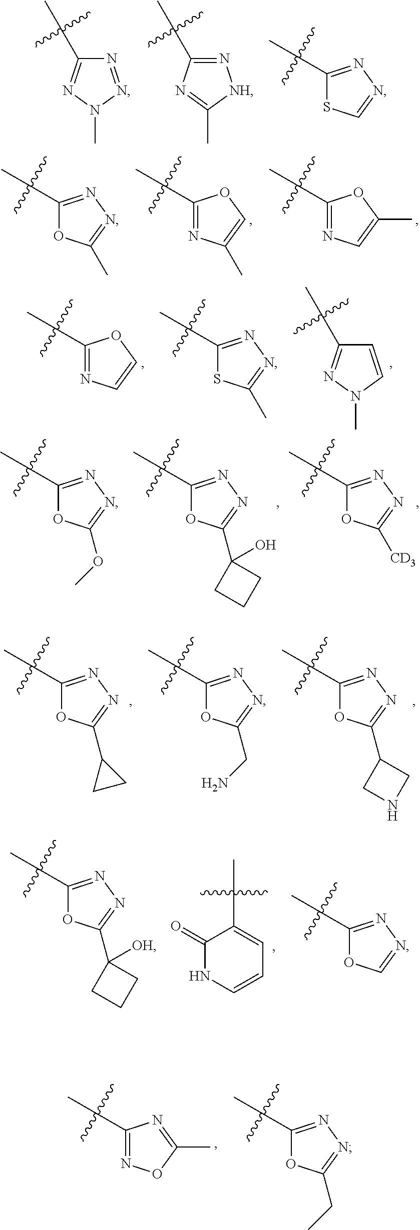

when R₁₂—Y₁ is selected from

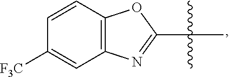 [SMILES: *c1nc2cc(C(F)(F)F)ccc2o1]

L₁ is a bond, L₂ is carbonyl, L₃ is

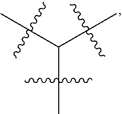 [SMILES: *C(*)*]

Y₃ is selected from

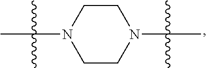 [SMILES: *N1CCN(*)CC1]

Y₄ is selected from a benzene ring and Y₅ is selected from

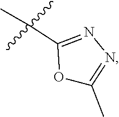 [SMILES: *c1nnc(C)o1]

Y₂ is not selected from the following groups:

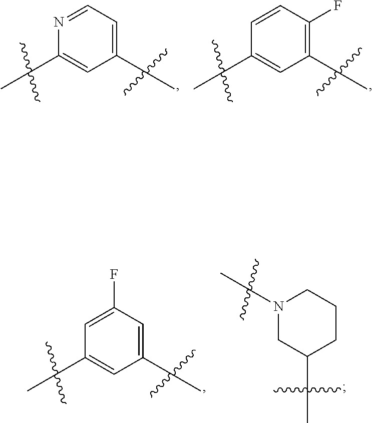 [SMILES: *c1ccnc(*)c1 ; *c1ccc(F)c(*)c1 ; *c1cc(*)cc(F)c1 ; *C1CCCN(*)C1]

when Y₅ is selected from acetamido, L₃ is a bond;
and when Y₅ is —C(O)OCH₂CH₃, it does not have the following structure:

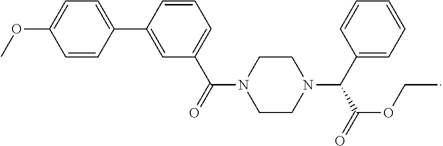 [SMILES: CCOC(=O)[C@@H](c1ccccc1)N1CCN(C(=O)c2cccc(-c3ccc(OC)cc3)c2)CC1]

For the compound of formula (I-a), the isomer thereof or the pharmaceutically acceptable salt thereof of the present disclosure,

Y₅ is selected from 5-15 membered heteroaryl and 3-12 membered heterocyclyl, wherein the heteroaryl and heterocyclyl are optionally substituted with 0-5 R^{y5}, and the definitions of other groups are consistent with those of embodiment five of the present disclosure.

Embodiment six of the present disclosure relates to a compound of formula (I-a), an isomer thereof or a pharmaceutically acceptable salt thereof, having a structure of formula (I-1),

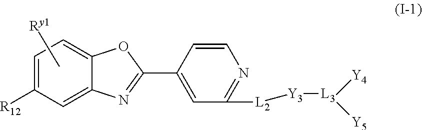

wherein Y₃ is selected from 3-12 membered heterocyclyl, 5-15 membered heteroaryl, a 5-12 membered fused ring, a 5-12 membered spiro ring or a 5-12 membered bridged ring, wherein the heterocyclyl, heteroaryl, fused ring, spiro ring and bridged ring are optionally further substituted with 0 to 3 of the following groups: halogen, hydroxyl, cyano, C₁₋₈ alkyl or C₁₋₈ haloalkyl; in certain embodiments, Y₃ is selected from 3-12 membered heterocyclyl, wherein the heterocyclyl is optionally further substituted with 0 to 3 of the following groups: halogen, hydroxyl, C₁₋₈ alkyl or C₁₋₈ haloalkyl; in certain embodiments, Y₃ is selected from a 5-12 membered fused ring, a 5-12 membered spiro ring or a 5-12 membered bridged ring, wherein the fused ring, spiro ring and bridged ring are optionally further substituted with 0 to 3 of the following groups: halogen and hydroxyl; in certain embodiments, Y₃ is selected from

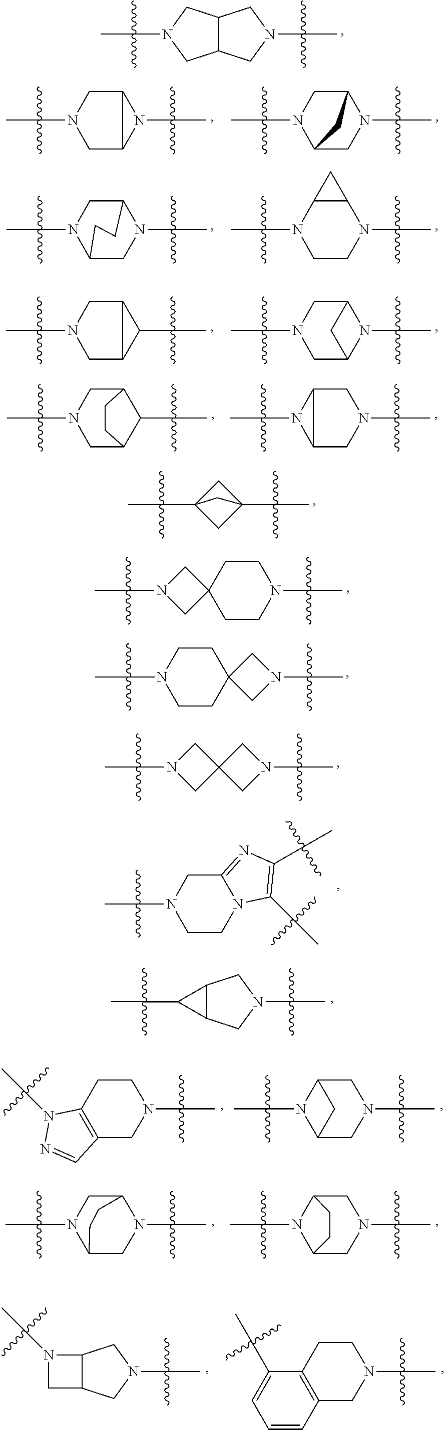

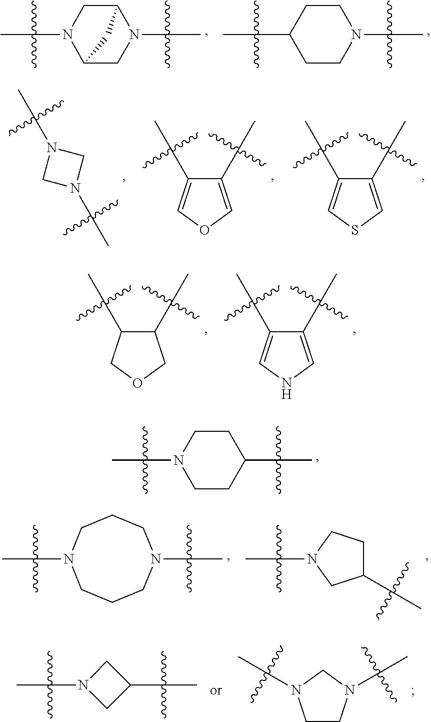

in certain embodiments, Y₃ is selected from

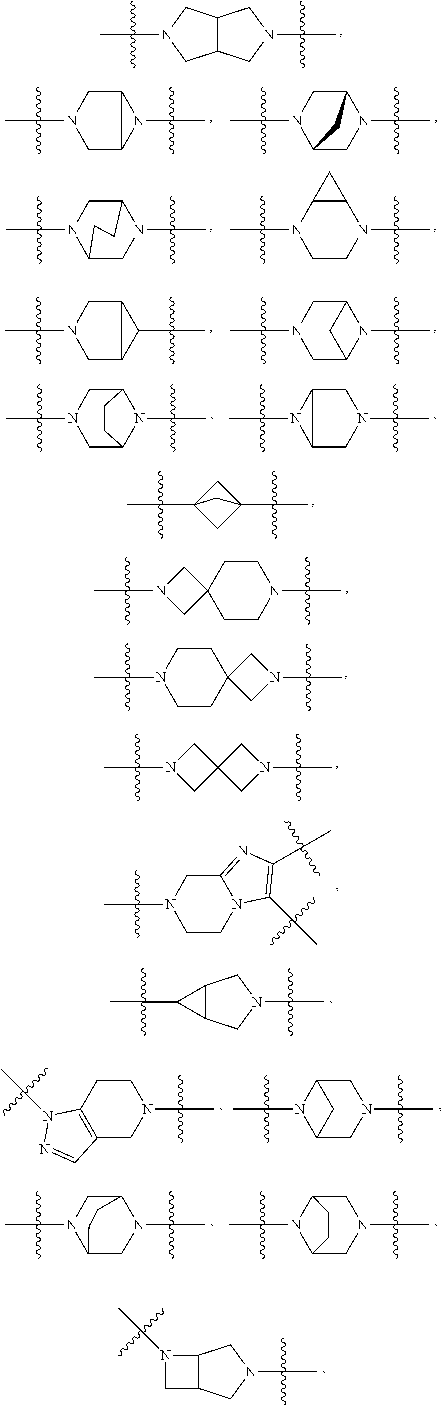

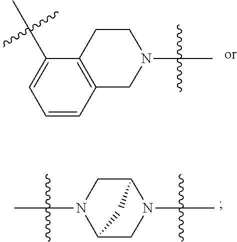 [SMILES: *c1cccc2c1CCN(*)C2 ; *N1C[C@@H]2C[C@H]1CN2*]

in certain embodiments, Y₃ is selected from

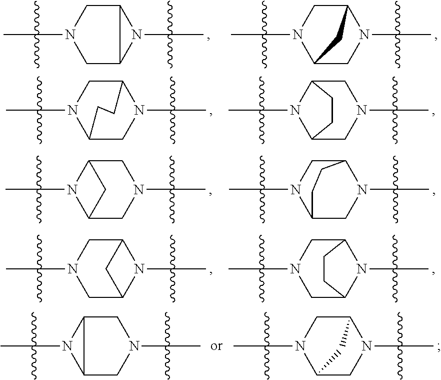

Y₄ is selected from C₅₋₁₅ aryl, 5-15 membered heteroaryl, 3-12 membered heterocyclyl, C₃₋₁₂ carbocyclyl, C₂₋₈ alkenyl or C₂₋₈ alkynyl, wherein the aryl, heteroaryl, heterocyclyl, carbocyclyl, alkenyl or alkynyl is optionally further substituted with 0 to 3 of the following groups: halogen, cyano, C₁₋₈ alkyl, C₃₋₈ cycloalkyl or C₁₋₈ haloalkyl; in certain embodiments, Y₄ is selected from C₅₋₁₅ aryl optionally further substituted with 0 to 3 of the following groups: halogen, cyano, C₁₋₈ alkyl, C₃₋₈ cycloalkyl or C₁₋₈ haloalkyl; in certain embodiments, Y₄ is selected from phenyl optionally further substituted with 0 to 3 of the following groups: halogen, cyano, C₁₋₈ alkyl, C₃₋₈ cycloalkyl or C₁₋₈ haloalkyl; in certain embodiments, Y₄ is selected from phenyl;
Y₅ is selected from 3-12 membered heterocyclyl or 5-15 membered heteroaryl, wherein the heterocyclyl and heteroaryl are optionally further substituted with 0 to 3 of the following groups: halogen, ═O, hydroxyl, cyano, nitro, C₁₋₈ hydroxylalkyl, C₁₋₈ alkyl, C₁₋₈ haloalkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl; in certain embodiments, Y₅ is selected from 5-15 membered heteroaryl, wherein the heteroaryl is optionally further substituted with 0 to 3 of the following groups: halogen, ═O, hydroxyl, cyano, nitro, C₁₋₈ alkyl, C₁₋₈ haloalkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl; in certain embodiments, Y₅ is selected from 5-15 membered heteroaryl, wherein the heteroaryl is optionally further substituted with 0 to 3 of the following groups: C₁₋₈ alkyl and C₁₋₈ haloalkyl; in certain embodiments, Y₅ is selected from

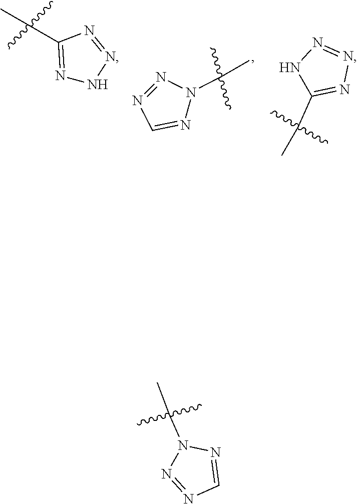 [SMILES: *c1nn[nH]n1 ; *n1ncnn1 ; *c1nnn[nH]1 ; *n1ncnn1]

optionally further substituted with 0 to 3 of the following groups: C₁₋₈ alkyl and C₁₋₈ haloalkyl; in certain embodiments, Y₅ is selected from

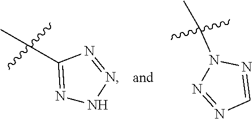 [SMILES: *c1nn[nH]n1 ; *n1ncnn1]

optionally further substituted with 0 to 3 of the following groups: C₁₋₈ alkyl and C₁₋₈ haloalkyl; in certain embodiments, Y₅ is selected from

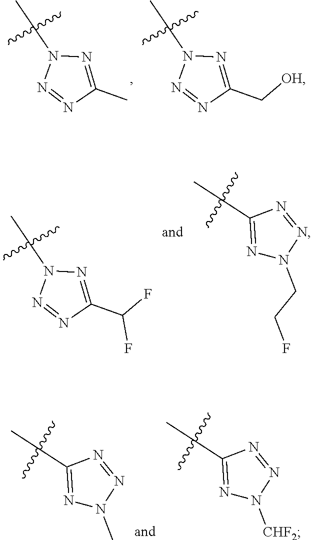

in certain embodiments, Y₅ is selected from

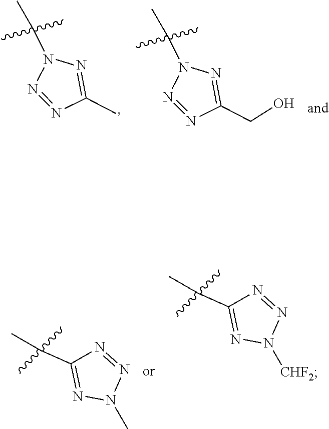

in certain embodiments, Y₅ is selected from

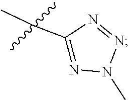 [SMILES: *c1nnn(C)n1]

in certain embodiments, Y₅ is selected from

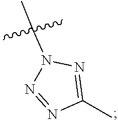 [SMILES: *n1nnc(C)n1]

embodiments, Y₅ is selected from

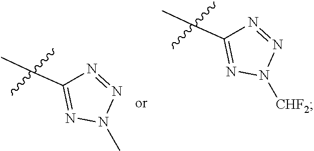

in certain embodiments, Y₅ is selected from

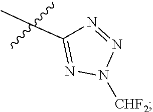 [SMILES: *c1nnn(C(F)F)n1]

R₁₂ is selected from 3-8 membered heterocyclyl, C₅₋₁₀ aryl or 5-10 membered heteroaryl or

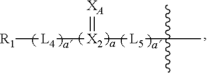

wherein the heterocyclyl, aryl and heteroaryl are optionally substituted with 0-3 of the following groups: halogen, ═O, acetamido, hydroxyl, cyano, sulfhydryl, nitro, C₁₋₈ alkyl, C₁₋₈ alkoxy or C₁₋₈ haloalkyl; in certain embodiments, R₁₂ is selected from 3-8 membered heterocyclyl and

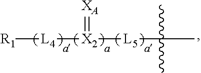

wherein the heterocyclyl is optionally substituted with 0-3 of the following groups: carbonyl; in certain embodiments, R₁₂ is selected from

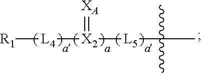

in certain embodiments, R₁₂ is selected from acetamido, cyclopropylcarboxamido and

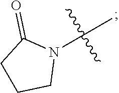 [SMILES: *N1CCCC1=O]

in certain embodiments, R₁₂ is selected from acetamido;
R^{y1} is selected from H, halogen, cyano or C₁₋₈ alkyl; in certain embodiments, R^{y1} is selected from H and halogen; in certain embodiments, R^{y1} is selected from H;
X₂ is selected from C;
L₄ and L₅ are each independently selected from: O, NR₂ or CR₃R₄; in certain embodiments, L₄ and L₅ are each independently selected from: O and NR₂;
X_{A} is selected from O and S; in certain embodiments, X_{A} is selected from O;
a is 0 or 1; in certain embodiments, a is 0; in certain embodiments, a is 1;
a′ is selected from 0, 1, 2, 3, 4, 5 or 6;
R₁ is selected from: H, C₁₋₈ alkyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, cycloalkyl, alkoxy, haloalkyl, heterocyclyl, aryl and heteroaryl are optionally further substituted with 0-5 groups selected from: hydroxyl, halogen, C₁₋₈ alkyl, cyano, ═O, C₁₋₈ alkoxy or C₁₋₈ haloalkyl; in certain embodiments, R₁ is selected from: C₁₋₈ alkyl and C₃₋₈ cycloalkyl, wherein the alkyl and cycloalkyl are optionally further substituted with 0-3 groups selected from: halogen, C₁₋₈ alkyl or C₁₋₈ haloalkyl; in certain embodiments, R₁ is selected from: C₁₋₈ alkyl, wherein the alkyl is optionally further substituted with 0-3 groups selected from: halogen, C₁₋₈ alkyl or C₁₋₈ haloalkyl; in certain embodiments, R₁ is selected from: C₁₋₈ alkyl, wherein the alkyl is optionally further substituted with 0-3 groups selected from: halogen;
R₂, R₃ and R₄ are each independently selected from: H, halogen, C₁₋₈ alkyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, optionally further substituted with 0-5 groups selected from: halogen, C₁₋₈ alkyl, C₁₋₈ alkoxy or C₁₋₈ haloalkyl; in certain embodiments, R₂, R₃ and R₄ are each independently selected from: H, halogen, C₁₋₈ alkyl and C₃₋₈ cycloalkyl, wherein the alkyl and cycloalkyl are optionally further substituted with 0-3 groups selected from: halogen; in certain embodiments, each R₂ is independently selected from: H and C₁₋₈ alkyl, and R₃ and R₄ are each independently selected from: H, halogen and C₁₋₈ alkyl, wherein the alkyl is optionally further substituted with 0-3 groups selected from: halogen; in certain embodiments, R₂, R₃ and R₄ are each independently selected from: H and C₁₋₈ alkyl; in certain embodiments, R₂, R₃ and R₄ are each independently selected from: H;
L₂ is selected from a bond and

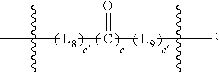

in certain embodiments, L₂ is selected from a bond, ═O, —C(O)NH— and —O—C(O)—; in certain embodiments, L₂ is selected from carbonyl and —C(O)NH—; in certain embodiments, L₂ is selected from carbonyl;
L₈ and L₉ are each independently selected from a bond, O, S, NR₅ or CR₆R₇; in certain embodiments, L₈ and L₉ are each independently selected from a bond, NR₅, O or CR₆R₇; in certain embodiments, L₈ and L₉ are each independently selected from a bond and NR₅; in certain embodiments, each L₈ is independently selected from a bond, and each L₉ is independently selected from a bond and NR₅;
c is selected from 1;
each c′ is independently selected from 0, 1, 2, 3, 4, 5 or 6;
R₅, R₆ and R₇ are each independently selected from H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl; in certain embodiments, R₅, R₆ and R₇ are each independently selected from H, halogen and C₁₋₈ alkyl; in certain embodiments, each R₅ is independently selected from H and C₁₋₈ alkyl, and R₆ and R₇ are each independently selected from H, halogen and C₁₋₈ alkyl; in certain embodiments, each R₅ is independently selected from H and C₁₋₈ alkyl, and R₆ and R₇ are each independently selected from H and C₁₋₈ alkyl; in certain embodiments, R₅, R₆ and R₇ are each independently selected from H;
L₃ is selected from a bond,

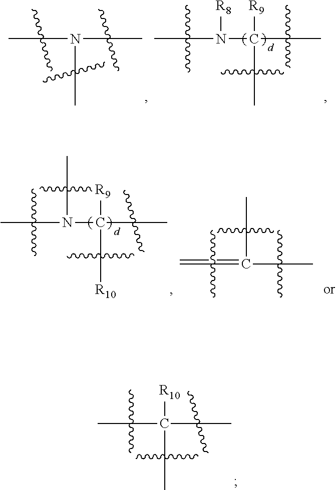

in certain embodiments, L₃ is selected from

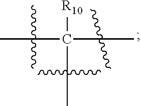

d is an integer selected from 1-6; in certain embodiments, d is selected from 1;
R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl; in certain embodiments, R₈, R₉ and R₁₀ are each independently selected from H, halogen and C₁₋₈ alkyl; in certain embodiments, R₈, R₉ and R₁₀ are each independently selected from H and halogen; and in certain embodiments, R₈, R₉ and R₁₀ are each independently selected from H; provided that: Y₃ is not selected from piperazinyl.

Embodiment six of the present disclosure relates to a compound having a structure of formula (I-1), an isomer thereof or a pharmaceutically acceptable salt thereof, wherein

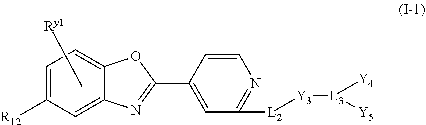

Y₃ is selected from 3-12 membered heterocyclyl, a 5-12 membered fused ring, a 5-12 membered spiro ring or a 5-12 membered bridged ring, wherein the heterocyclyl, fused ring, spiro ring and bridged ring are optionally further substituted with 0 to 3 of the following groups: halogen, hydroxyl, cyano, C₁₋₈ alkyl or C₁₋₈ haloalkyl;
Y₄ is selected from C₅₋₁₅ aryl, 5-15 membered heteroaryl, 3-12 membered heterocyclyl, C₃₋₁₂ carbocyclyl, C₂₋₈ alkenyl or C₂₋₈ alkynyl, wherein the aryl, heteroaryl, heterocyclyl, carbocyclyl, alkenyl or alkynyl is optionally further substituted with 0 to 3 of the following groups: halogen, cyano, C₁₋₈ alkyl, C₃₋₈ cycloalkyl or C₁₋₈ haloalkyl; Y₅ is selected from 3-12 membered heterocyclyl or 5-15 membered heteroaryl, wherein the heterocyclyl and heteroaryl are optionally further substituted with 0 to 3 of the following groups: halogen, ═O, hydroxyl, cyano, nitro, C₁₋₈ hydroxylalkyl, C₁₋₈ alkyl, C₁₋₈ haloalkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁₂ is selected from 3-8 membered heterocyclyl, C₅₋₁₀ aryl or 5-10 membered heteroaryl or

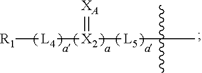

wherein the heterocyclyl, aryl and heteroaryl are optionally substituted with 0-3 of the following groups: halogen, ═O, acetamido, hydroxyl, cyano, sulfhydryl, nitro, C₁₋₈ alkyl, C₁₋₈ alkoxy or C₁₋₈ haloalkyl;
R^{y1} is selected from H, halogen, cyano or C₁₋₈ alkyl;
X₂ is selected from C;
L₄ and L₅ are each independently selected from: O, NR₂ or CR₃R₄;
X_{A} is selected from O and S;
a is 0 or 1;
a′ is selected from 0, 1, 2, 3, 4, 5 or 6;
R₁ is selected from: H, C₁₋₈ alkyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, cycloalkyl, alkoxy, haloalkyl, heterocyclyl, aryl and heteroaryl are optionally further substituted with 0-5 groups selected from: hydroxyl, halogen, C₁₋₈ alkyl, cyano, ═O, C₁₋₈ alkoxy or C₁₋₈ haloalkyl;
R₂, R₃ and R₄ are each independently selected from: H, halogen, C₁₋₈ alkyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, optionally further substituted with 0-5 groups selected from: halogen, C₁₋₈ alkyl, C₁₋₈ alkoxy or C₁₋₈ haloalkyl;
L₂ is selected from a bond and

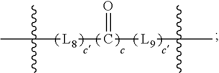

L₈ and L₉ are each independently selected from a bond, O, S, NR₅ or CR₆R₇;
c is selected from 1;
each c′ is independently selected from 0, 1, 2, 3, 4, 5 or 6;
R₅, R₆ and R₇ are each independently selected from H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl;
L₃ is selected from a bond

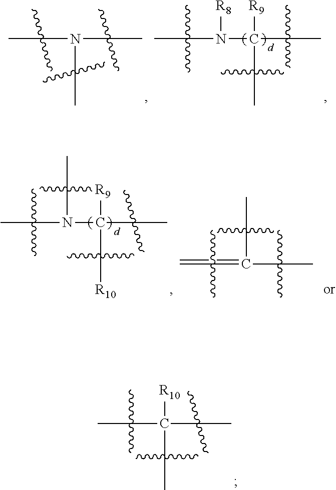

d is an integer selected from 1-6;
and R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl;
provided that: Y₃ is not selected from piperazinyl.

Embodiment seven of the present disclosure relates to a compound of formula (I-1), an isomer thereof or a pharmaceutically acceptable salt thereof, wherein Y₃ is selected from

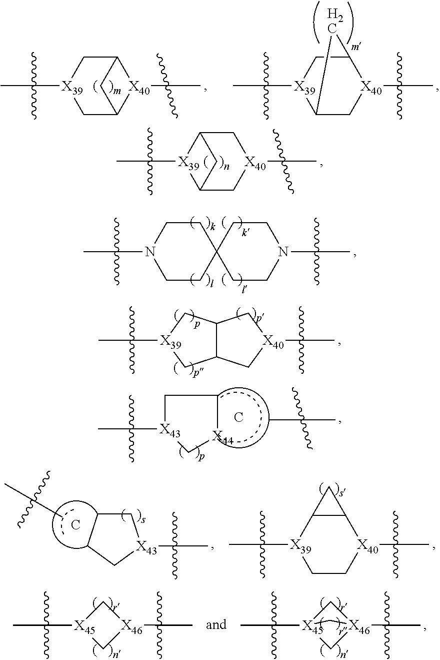

wherein the above-mentioned groups can be optionally substituted with 0-3 substituents selected from: halogen, hydroxyl, cyano, C₁₋₈ alkyl or C₁₋₈ haloalkyl;
p, m, n, s, s′, r′, r″, n′, r, p′ and p″ are each independently selected from 0, 1 and 2; m′ is selected from 1 or 2;
k, k′, 1 and 1′ are each independently selected from: 0 or 1;
X₄₃ and X₄₄ are each independently selected from: CR₂₇R₂₇ₐ or NR₂₈;
X₃₉ and X₄₀ are each independently selected from: CR₂₃R₂₃ₐ or NR₂₄;
X₄₅ and X₄₆ are each independently selected from: CR₂₃R₂₃ₐ or NR₂₄; ring C is C₃₋₈ carbocyclyl, 3-8 membered heterocyclyl or C₅₋₁₀ aryl;
R₂₃, R₂₃ₐ and R₂₄ are each independently selected from H, halogen, hydroxyl, cyano, C₁₋₈ alkyl or C₁₋₈ haloalkyl;
and R₂₇, R₂₇ₐ and R₂₈ are each independently selected from H, halogen, hydroxyl, cyano, C₁₋₈ alkyl or C₁₋₈ haloalkyl;
provided that: Y₃ is not selected from piperazinyl; and the definitions of other groups are consistent with those of embodiment six of the present disclosure.

For the compound of formula (I-1), the isomer thereof or the pharmaceutically acceptable salt thereof of the present disclosure, Y₃ is selected from

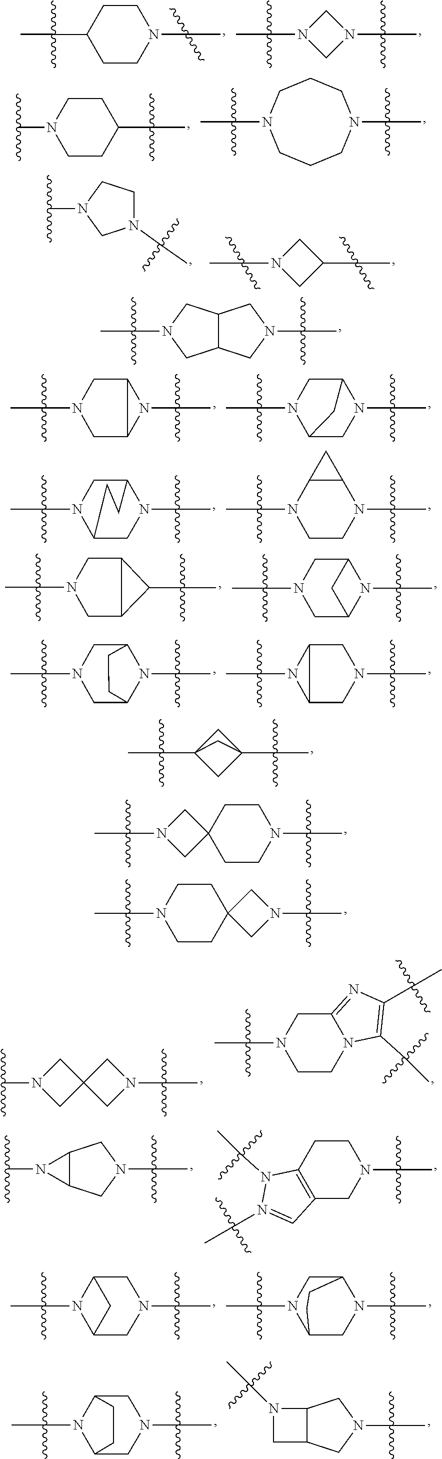

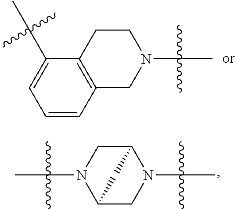

wherein the above-mentioned groups are optionally further substituted with 0 to 3 of the following groups: halogen, hydroxyl, cyano, C₁₋₈ alkyl or C₁₋₈ haloalkyl, and other groups are consistent with those of embodiment six of the present disclosure; or other groups are consistent with those of embodiment seven of the present disclosure.

For the compound of formula (I-1), the isomer thereof or the pharmaceutically acceptable salt thereof of the present disclosure, Y₃ is selected from

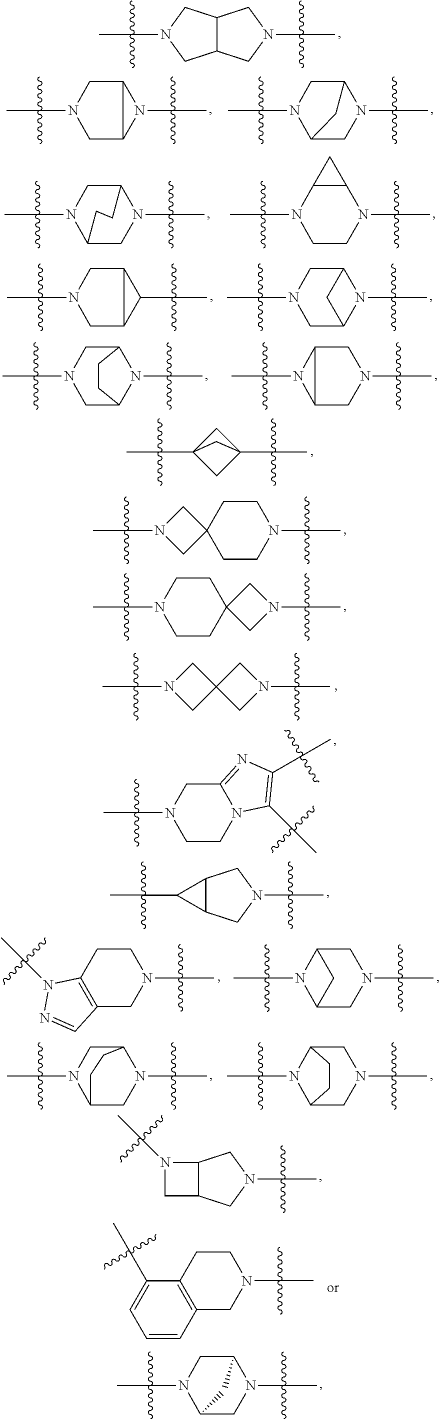

wherein the above-mentioned groups are optionally further substituted with 0 to 3 of the following groups: halogen, hydroxyl, cyano, C₁₋₈ alkyl or C₁₋₈ haloalkyl; and other groups are consistent with those of embodiment six of the present disclosure.

For the compound of formula (I-1), the isomer thereof or the pharmaceutically acceptable salt thereof of the present disclosure, Y₃ is selected from

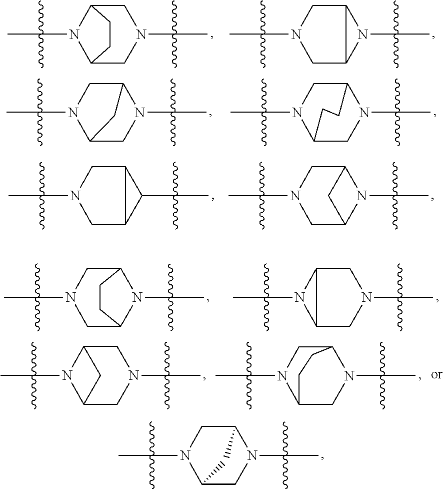

wherein the above-mentioned groups are optionally further substituted with 0 to 3 of the following groups: halogen, hydroxyl, cyano, C₁₋₈ alkyl or C₁₋₈ haloalkyl; and other groups are consistent with those of embodiment six of the present disclosure.

For the compound of formula (I-1), the isomer thereof or the pharmaceutically acceptable salt thereof of the present disclosure, Y₃ is selected from

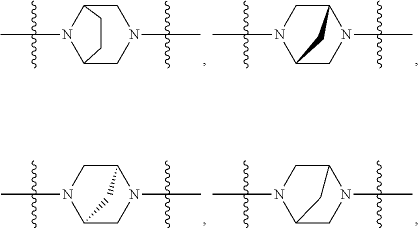 [SMILES: *N1CC2CCC(C1)N2* ; *N1C[C@H]2C[C@@H]1CN2* ; *N1C[C@@H]2C[C@H]1CN2* ; *N1CC2CC1CN2*]

wherein the above-mentioned groups are optionally further substituted with 0 to 3 of the following groups: halogen, hydroxyl, cyano, C₁₋₈ alkyl or C₁₋₈ haloalkyl; and other groups are consistent with those of embodiment six of the present disclosure.

For the compound of formula (I-1), the isomer thereof or the pharmaceutically acceptable salt thereof of the present disclosure,

Y₄ is selected from phenyl; Y₅ is selected from

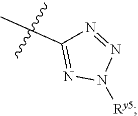

R^{y5} is selected from methyl or fluoroethyl;
L₂ is selected from carbonyl; L₃ is selected from

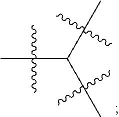 [SMILES: *C(*)*]

R₁₂ is selected from acetamido, cyclopropylacetamido, fluorocyclopropylacetamido or

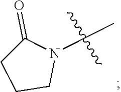 [SMILES: *N1CCCC1=O]

R^{y1} is selected from H, and the definition of Y₃ is consistent with that of embodiment six; or the definition of Y₃ is consistent with that of embodiment seven.

For the compound of formula (I-1), the isomer thereof or the pharmaceutically acceptable salt thereof of the present disclosure,

Y₄ is selected from phenyl; Y₅ is selected from

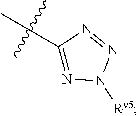

R^{y5} is selected from methyl or fluoroethyl; L₂ is selected from carbonyl;
L₃ is selected from

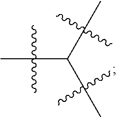 [SMILES: *C(*)*]

R₁₂ is selected from acetamido, cyclopropylacetamido, fluorocyclopropylacetamido or

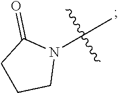 [SMILES: *N1CCCC1=O]

R^{y1} is selected from H;
and Y₃ is selected from

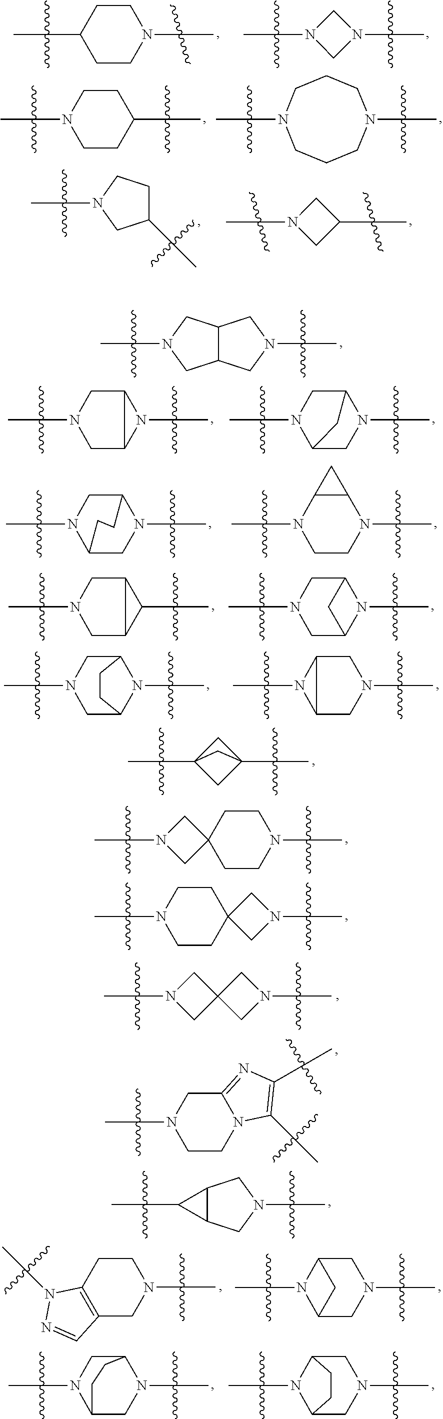

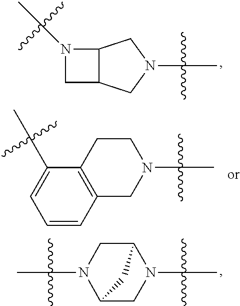

wherein the above-mentioned groups are optionally further substituted with 0 to 3 of the following groups: halogen, hydroxyl, cyano, C₁₋₈ alkyl or C₁₋₈ haloalkyl.

For the compound of formula (I-1), the isomer thereof or the pharmaceutically acceptable salt thereof of the present disclosure,

Y₄ is selected from phenyl; Y₅ is selected from

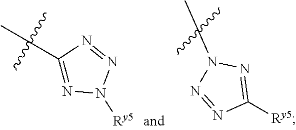

R^{y5} is selected from methyl or fluoroethyl; L₂ is selected from carbonyl;
L₃ is selected from

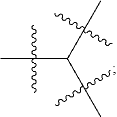 [SMILES: *C(*)*]

R₁₂ is selected from acetamido, cyclopropylacetamido, fluorocyclopropylacetamido or

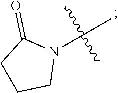 [SMILES: *N1CCCC1=O]

R^{y1} is selected from H;
and Y₃ is selected from

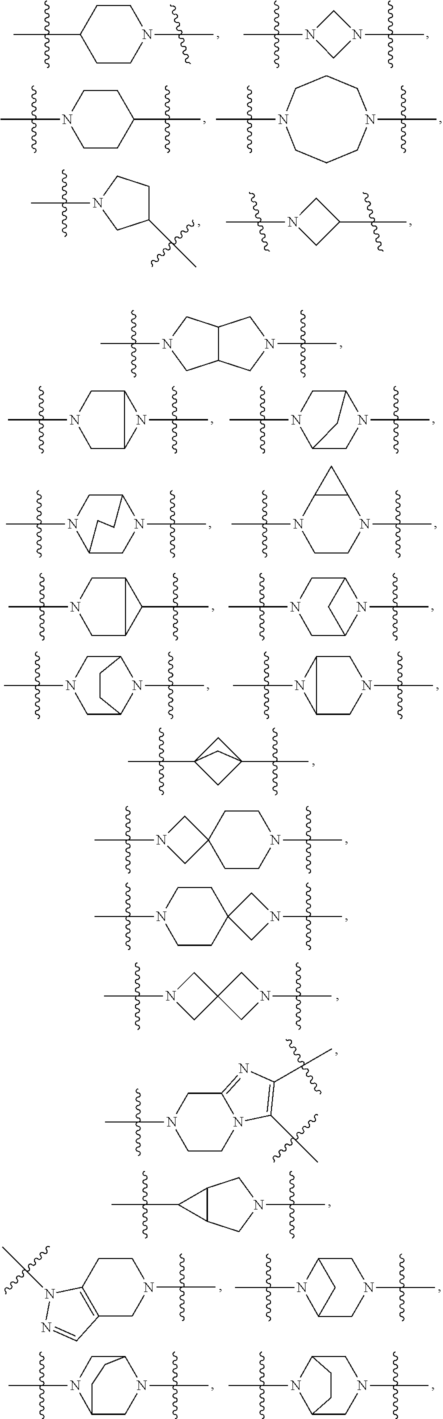

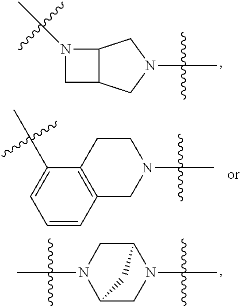

wherein the above-mentioned groups are optionally further substituted with 0 to 3 of the following groups: halogen, hydroxyl, cyano, C₁₋₈ alkyl or C₁₋₈ haloalkyl.

For the compound of formula (I-1), the isomer thereof or the pharmaceutically acceptable salt thereof of the present disclosure,

Y₄ is selected from phenyl; Y₅ is selected from

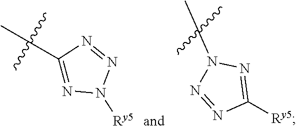

R^{y5} is selected from methyl or fluoroethyl; L₂ is selected from carbonyl;
L₃ is selected from

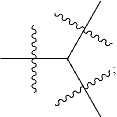 [SMILES: *C(*)*]

R₁₂ is selected from acetamido, cyclopropylacetamido, fluorocyclopropylacetamido or

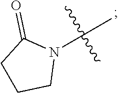 [SMILES: *N1CCCC1=O]

R^{y1} is selected from H;
and Y₃ is selected from

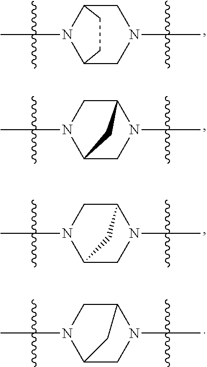

Embodiment eight of the present disclosure relates to a compound of formula (I-a), an isomer thereof or a pharmaceutically acceptable salt thereof, wherein Y₄ is selected from phenyl optionally substituted with 0-5 R⁴⁴;

and Y₅ is selected from tetrazolyl and

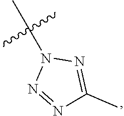 [SMILES: *n1nnc(C)n1]

wherein the tetrazolyl is further substituted with difluoromethyl, fluoroethyl and hydroxymethyl, and the definitions of other groups are consistent with those of embodiment five of the present disclosure.

Embodiment nine of the present disclosure relates to a compound of formula (I-a), an isomer thereof or a pharmaceutically acceptable salt thereof, which has a structure of formula (I-2),

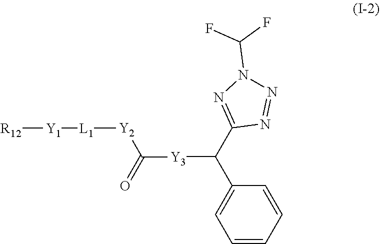

wherein,
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1}; in certain embodiments, Y₁ is selected from C₅₋₁₅ aryl, 5-15 membered heteroaryl and 3-12 membered heterocyclyl, wherein the aryl, heteroaryl and heterocyclyl are optionally substituted with 0-5 R^{y1}; in certain embodiments, Y₁ is selected from 5-15 membered heteroaryl and 3-12 membered heterocyclyl, wherein the heteroaryl and heterocyclyl are optionally substituted with 0-5 R^{y1}; in certain embodiments, Y₁ is selected from phenyl and

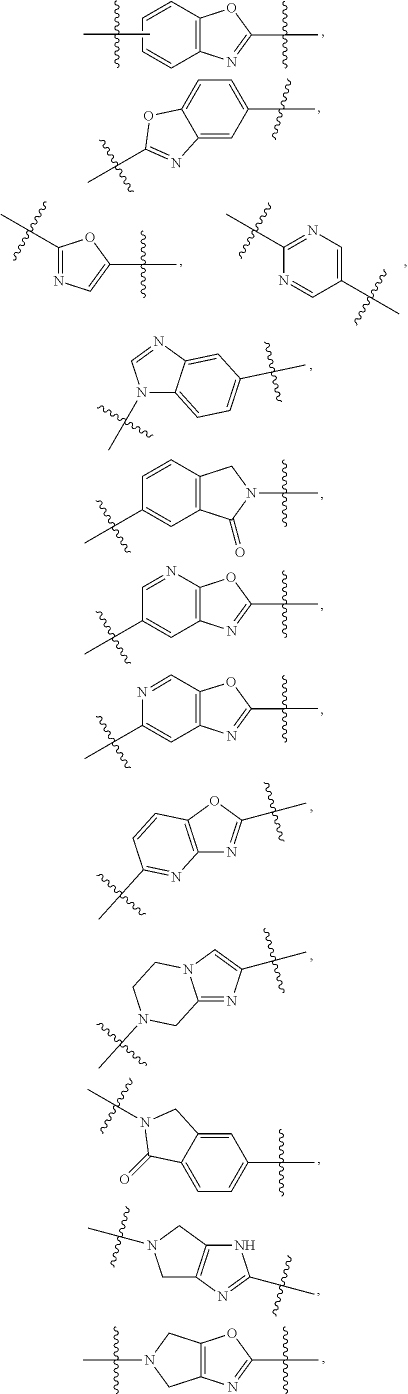

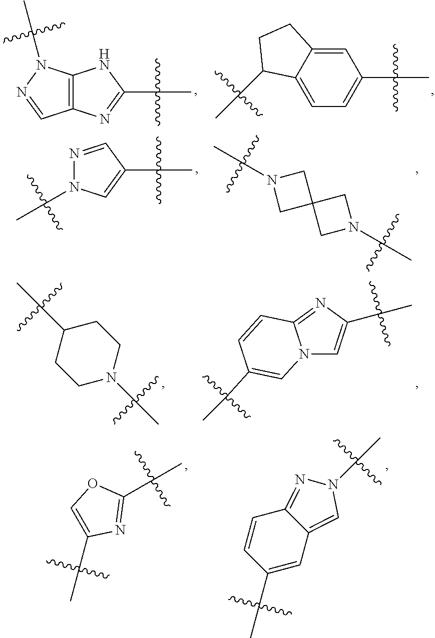

wherein the above-mentioned groups are optionally substituted with 0-3 R^{y1}; in certain embodiments, Y₁ is selected from

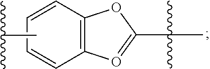

in certain embodiments, Y₁ is selected from

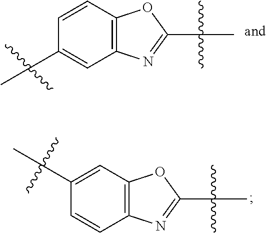 [SMILES: *c1ccc2oc(*)nc2c1 ; *c1ccc2nc(*)oc2c1]

in certain embodiments, Y₁ is selected from

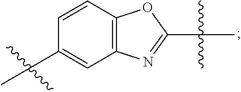 [SMILES: *c1ccc2oc(*)nc2c1]

R₁₂ is selected from cyano,

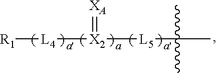

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, sulfhydryl, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; in certain embodiments, R₁₂ is selected from

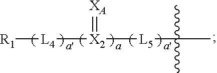

in certain embodiments, R₁₂ is selected from 3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, sulfhydryl, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; in certain embodiments, R₁₂ is selected from 3-8 membered heterocyclyl, C₅₋₁₀ aryl or 5-10 membered heteroaryl, wherein the heterocyclyl, aryl and heteroaryl are optionally substituted with 0-3 of the following groups: halogen, ═O, acetamido and C₁₋₈ alkyl; in certain embodiments, R₁₂ is selected from 3-8 membered heterocyclyl and 5-10 membered heteroaryl, wherein the heterocyclyl and heteroaryl are optionally substituted with 0-3 of the following groups: halogen, ═O, acetamido and C₁₋₈ alkyl; in certain embodiments, R₁₂ is selected from 3-8 membered heterocyclyl, wherein the heterocyclyl is optionally substituted with 0-3 of the following groups: halogen, ═O, acetamido and C₁₋₈ alkyl; in certain embodiments, R₁₂ is selected from 5-10 membered heteroaryl, wherein the heterocyclyl is optionally substituted with 0-3 of the following groups: halogen, ═O, acetamido and C₁₋₈ alkyl;
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl; in certain embodiments, R^{y1} and R^{y2} together with L₁ form

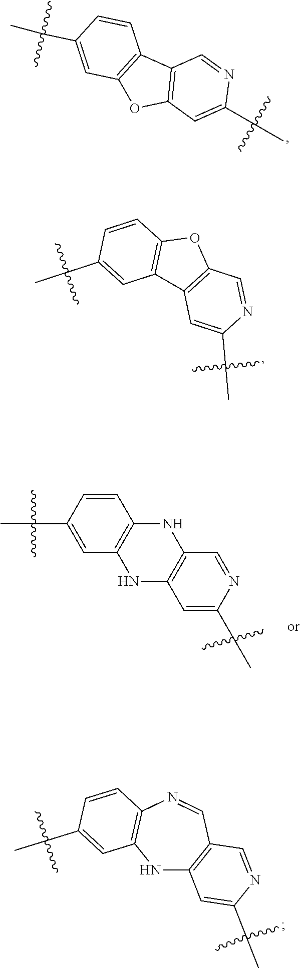 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

X₂ is selected from C;
L₄ and L₅ are each independently selected from: a bond, NR₂, CR₃R₄, O, or S; in certain embodiments, L₄ and L₅ are each independently selected from: a bond, 0 and NR₂; in certain embodiments, L₄ and L₅ are each independently selected from: a bond and NR₂; in certain embodiments, L₄ and L₅ are each independently selected from: 0 and NR₂; in certain embodiments, L₄ and L₅ are each independently selected from: NR₂;
X_{A} is selected from O and S; a is 0 or 1; in certain embodiments, X_{A} is selected from O;
a′ is selected from 0, 1, 2, 3, 4, 5 or 6; in certain embodiments, a′ is selected from 0 and 1; in certain embodiments, a′ is selected from 1;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; in certain embodiments, R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: hydroxyl, halogen, cyano, ═O, C₁₋₈ alkyl, C₁₋₈ alkoxy or C₁₋₈ haloalkyl; in certain embodiments, R₁ is selected from C₁₋₈ alkyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, cycloalkyl, alkoxy, haloalkyl, heterocyclyl, aryl or heteroaryl is optionally further substituted with 0-5 groups selected from: halogen, cyano, ═O, C₁₋₈ alkyl, C₁₋₈ alkoxy or C₁₋₈ haloalkyl; in certain embodiments, R₁ is selected from C₁₋₈ alkyl, C₃₋₈ cycloalkyl and 3-8 membered heterocyclyl, wherein the alkyl, cycloalkyl and heterocyclyl are optionally further substituted with 0-5 groups selected from: halogen, cyano, ═O, C₁₋₈ alkyl, C₁₋₈ alkoxy or C₁₋₈ haloalkyl; in certain embodiments, R₁ is selected from C₁₋₈ alkyl and C₃₋₈ cycloalkyl, wherein the alkyl and cycloalkyl are optionally further substituted with 0-5 groups selected from: halogen, cyano, ═O, C₁₋₈ alkyl, C₁₋₈ alkoxy or C₁₋₈ haloalkyl; in certain embodiments, R₁ is selected from C₁₋₈ alkyl; in certain embodiments, R₁ is selected from C₃₋₈ cycloalkyl, wherein the cycloalkyl is optionally further substituted with 0-5 groups selected from: halogen, cyano, ═O, C₁₋₈ alkyl, C₁₋₈ alkoxy or C₁₋₈ haloalkyl; in certain embodiments, R₁ is selected from 3-8 membered heterocyclyl, wherein the heterocyclyl is optionally further substituted with 0-5 groups selected from: halogen, cyano, ═O, C₁₋₈ alkyl, C₁₋₈ alkoxy or C₁₋₈ haloalkyl;
R₂, R₃ and R₄ are each independently selected from: H, halogen, C₁₋₈ alkyl or C₁₋₈ haloalkyl; in certain embodiments, each R₂ is independently selected from: H and C₁₋₈ alkyl, and R₃ and R₄ are each independently selected from: H, halogen, C₁₋₈ alkyl or C₁₋₈ haloalkyl; in certain embodiments, each R₂ is independently selected from: H and C₁₋₈ alkyl, and R₃ and R₄ are each independently selected from: H and C₁₋₈ alkyl; in certain embodiments, each R₂ is independently selected from: H, and R₃ and R₄ are each independently selected from H;
and the definitions of other groups are consistent with those of embodiment five of the present disclosure.

In certain embodiments, embodiment nine of the present disclosure relates to a compound represented by general formula (I-a), an isomer thereof or a pharmaceutically acceptable salt thereof, which has a structure of formula (I-2),

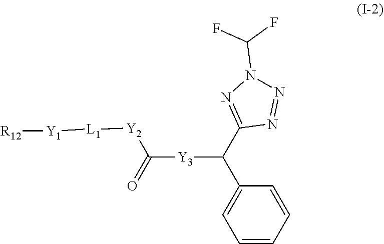

wherein,
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R₁₂ is selected from cyano,

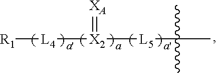

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
X₂ is selected from C;
L₄ and L₅ are each independently selected from: a bond, NR₂, CR₃R₄, O, or S;
X_{A} is selected from O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; in certain embodiments, R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: hydroxyl, halogen, cyano, ═O, C₁₋₈ alkyl, C₁₋₈ alkoxy or C₁₋₈ haloalkyl; R₂, R₃ and R₄ are each independently selected from: H, halogen, C₁₋₈ alkyl or C₁₋₈ haloalkyl;
and the definitions of other groups are consistent with those of embodiment five of the present disclosure.

For the compound of formula (I-2), the isomer thereof or the pharmaceutically acceptable salt thereof of the present disclosure, Y₃ is selected from

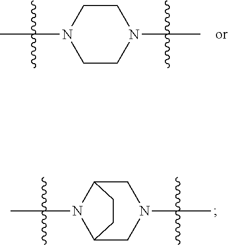 [SMILES: *N1CCN(*)CC1 ; *N1CC2CCC(C1)N2*]

and the definitions of other groups are consistent with those of embodiment nine of the present disclosure.

For the compound of formula (I-2), the isomer thereof or the pharmaceutically acceptable salt thereof of the present disclosure, Y₃ is selected from

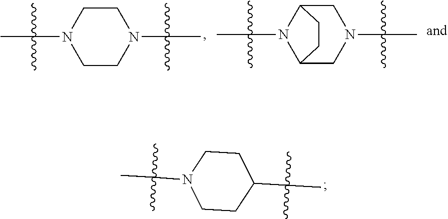 [SMILES: *N1CCN(*)CC1 ; *N1CC2CCC(C1)N2* ; *C1CCN(*)CC1]

and the definitions of other groups are consistent with those of embodiment nine of the present disclosure.

For the compound of formula (I-2), the isomer thereof or the pharmaceutically acceptable salt thereof of the present disclosure, Y₂ is selected from

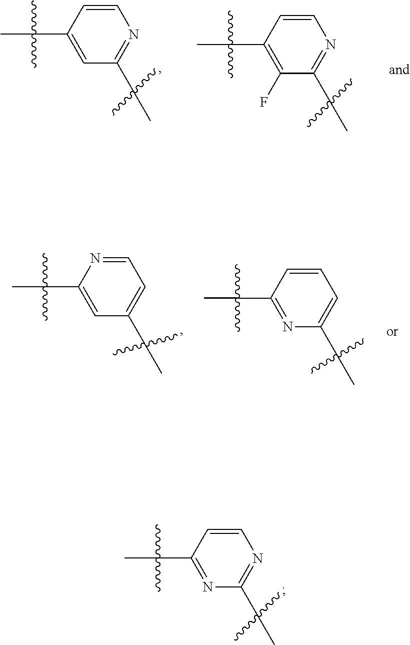 [SMILES: *c1ccnc(*)c1 ; *c1ccnc(*)c1F ; *c1ccnc(*)c1 ; *c1cccc(*)n1 ; *c1ccnc(*)n1]

and the definitions of other groups are consistent with those of embodiment nine of the present disclosure.

For the compound of formula (I-2), the isomer thereof or the pharmaceutically acceptable salt thereof of the present disclosure, L₁ is selected from a bond,

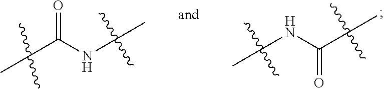 [SMILES: *NC(*)=O ; *NC(*)=O]

and the definitions of other groups are consistent with those of embodiment nine of the present disclosure.

For the compound of formula (I-2), the isomer thereof or the pharmaceutically acceptable salt thereof of the present disclosure,

Y₃ is selected from

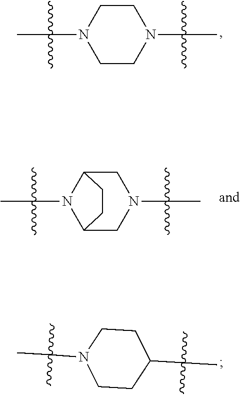 [SMILES: *N1CCN(*)CC1 ; *N1CC2CCC(C1)N2* ; *C1CCN(*)CC1]

in certain embodiments,
Y₃ is selected from

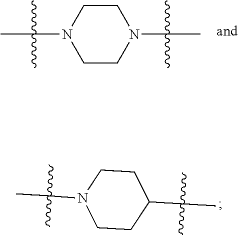 [SMILES: *N1CCN(*)CC1 ; *C1CCN(*)CC1]

in certain embodiments, Y₃ is selected from

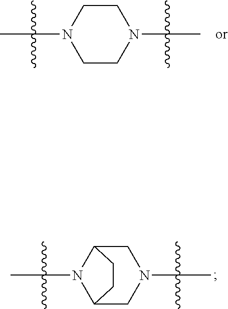 [SMILES: *N1CCN(*)CC1 ; *N1CC2CCC(C1)N2*]

in certain embodiments, Y₃ is selected from

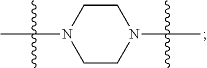 [SMILES: *N1CCN(*)CC1]

Y₂ is selected from

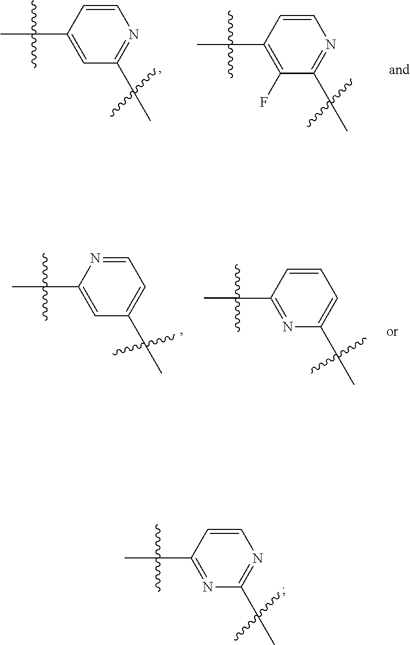 [SMILES: *c1ccnc(*)c1 ; *c1ccnc(*)c1F ; *c1ccnc(*)c1 ; *c1cccc(*)n1 ; *c1ccnc(*)n1]

in certain embodiments, Y₂ is selected from

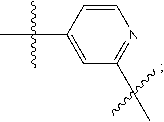 [SMILES: *c1ccnc(*)c1]

L₁ is selected from a bond,

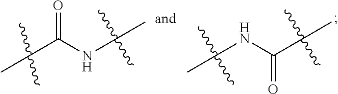 [SMILES: *NC(*)=O ; *NC(*)=O]

in certain embodiments, L₁ is selected from a bond and

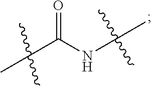 [SMILES: *NC(*)=O]

in certain embodiments, L₁ is selected from a bond; Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R₁₂ is selected from

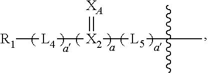

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
X₂ is selected from C;
L₄ and L₅ are each independently selected from: a bond, NR₂, CR₃R₄, O, or S;
X_{A} is selected from O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
and R₂, R₃ and R₄ are each independently selected from: H, halogen, C₁₋₈ alkyl or C₁₋₈ haloalkyl.

For the compound of formula (I-2), the isomer thereof or the pharmaceutically acceptable salt thereof of the present disclosure, Y₃ is selected from

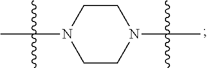 [SMILES: *N1CCN(*)CC1]

and the definitions of other groups are consistent with those of embodiment nine of the present disclosure.

For the compound of formula (I-2), the isomer thereof or the pharmaceutically acceptable salt thereof of the present disclosure, Y₂ is selected from

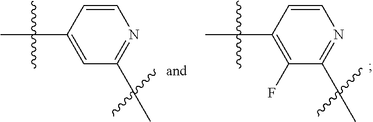 [SMILES: *c1ccnc(*)c1 ; *c1ccnc(*)c1F]

and the definitions of other groups are consistent with those of embodiment nine of the present disclosure.

For the compound of formula (I-2), the isomer thereof or the pharmaceutically acceptable salt thereof of the present disclosure, L₁ is selected from a bond; and the definitions of other groups are consistent with those of embodiment nine of the present disclosure.

For the compound of formula (I-2), the isomer thereof or the pharmaceutically acceptable salt thereof of the present disclosure,

Y₃ is selected from

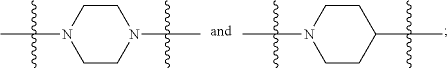 [SMILES: *N1CCN(*)CC1 ; *C1CCN(*)CC1]

in certain embodiments, Y₃ is selected from

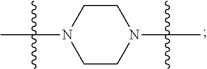 [SMILES: *N1CCN(*)CC1]

Y₂ is selected from

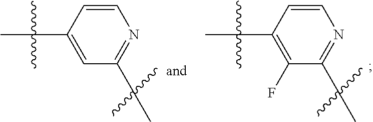 [SMILES: *c1ccnc(*)c1 ; *c1ccnc(*)c1F]

in certain embodiments, Y₂ is selected from

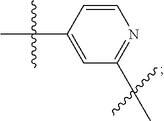 [SMILES: *c1ccnc(*)c1]

L₁ is selected from a bond;
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R₁₂ is selected from

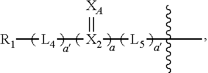

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
X₂ is selected from C;
L₄ and L₅ are each independently selected from: a bond, NR₂, CR₃R₄, O, or S;
X_{A} is selected from O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
and R₂, R₃ and R₄ are each independently selected from: H, halogen, C₁₋₈ alkyl or C₁₋₈ haloalkyl.

For the compound of formula (I-2), the isomer thereof or the pharmaceutically acceptable salt thereof of the present disclosure,

Y₁ is selected from phenyl,

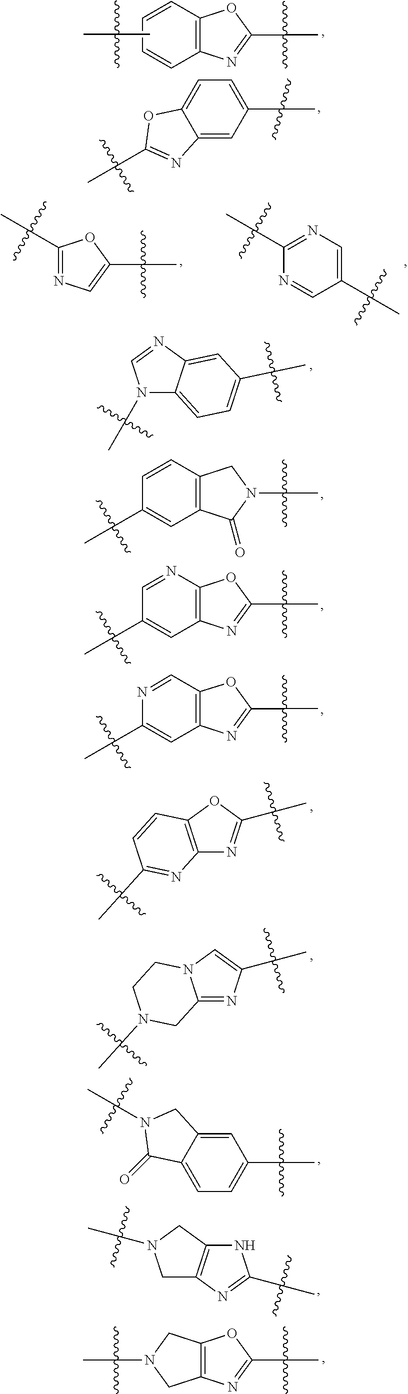

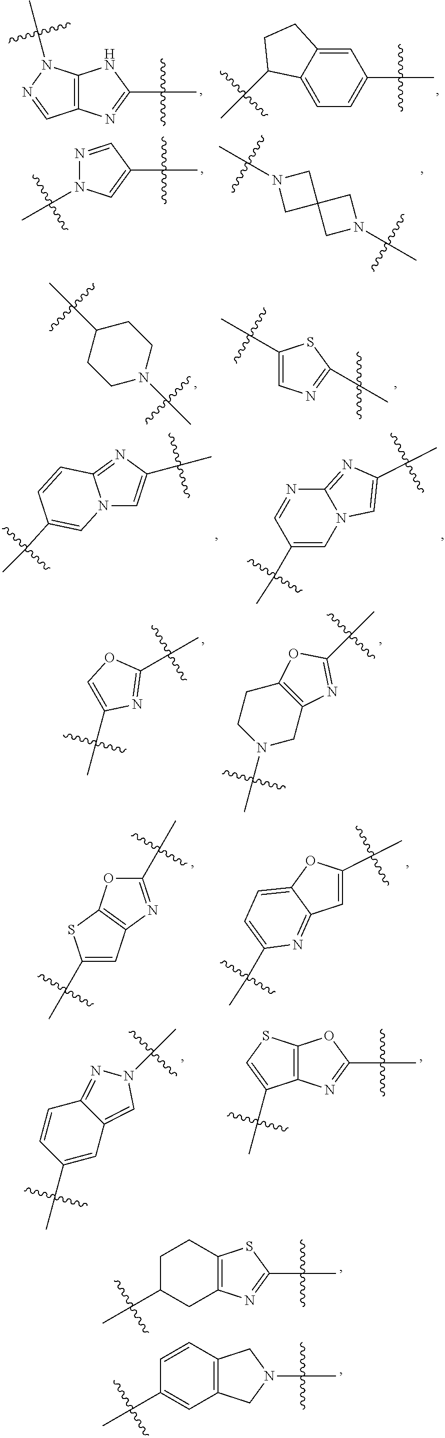

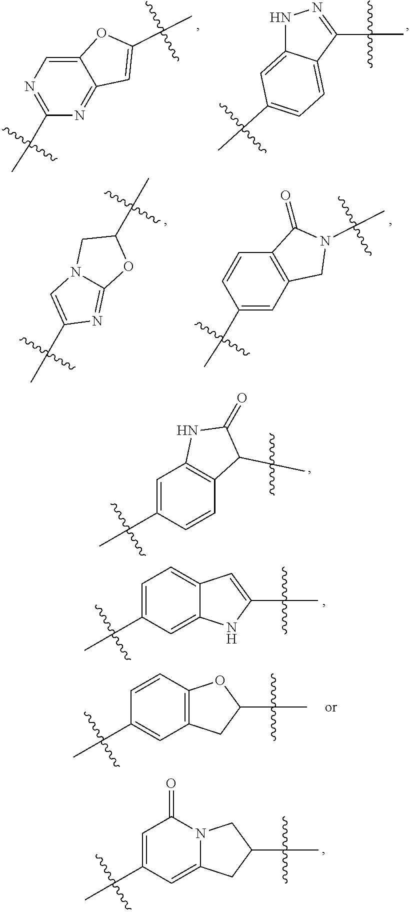

wherein the above-mentioned groups are optionally substituted with 0-3 R^{y1}; or
R^{y1} and R^{y2} together with L₁ form the following structure:

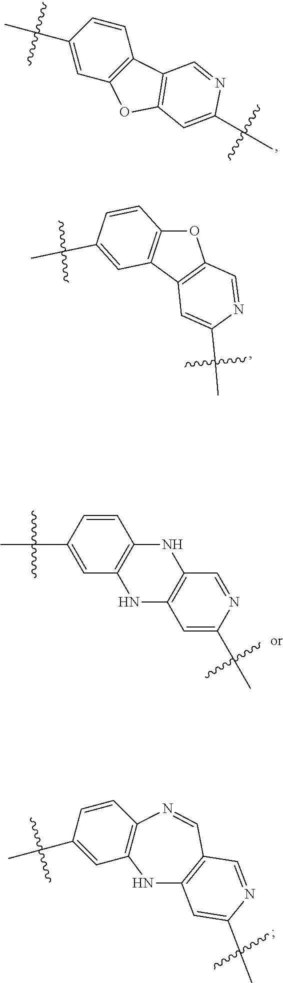 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

R₁₂ is selected from cyano,

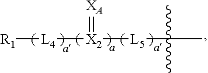

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₂ is selected from C;
L₄ and L₅ are each independently selected from: a bond, NR₂, CR₃R₄, O, or S;
X_{A} is selected from O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
and R₂, R₃ and R₄ are each independently selected from: H, halogen, C₁₋₈ alkyl or C₁₋₈ haloalkyl.

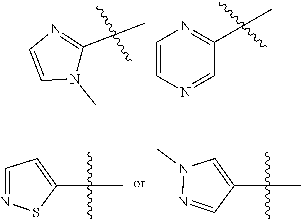

For the compound of formula (I-2), the isomer thereof or the pharmaceutically acceptable salt thereof of the present disclosure,
Y₁ is selected from

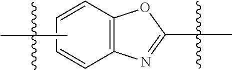

and optionally substituted with 0-3 R^{y1}; in certain embodiments, Y₁ is selected from

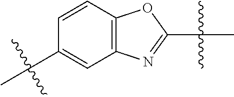 [SMILES: *c1ccc2oc(*)nc2c1]

and optionally substituted with 0-3 R^{y1};
R^{y1} is selected from F, methyl, difluoromethyl or trifluoromethyl;
R₁₂ is selected from cyano,

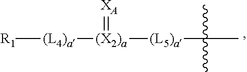

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₂ is selected from C;
L₄ and L₅ are each independently selected from: a bond, NR₂, CR₃R₄, O, or S;
X_{A} is selected from O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
and R₂, R₃ and R₄ are each independently selected from: H, halogen, C₁₋₈ alkyl or C₁₋₈ haloalkyl.

For the compound of formula (I-2), the isomer thereof or the pharmaceutically acceptable salt thereof of the present disclosure,

R₁₂ is selected from

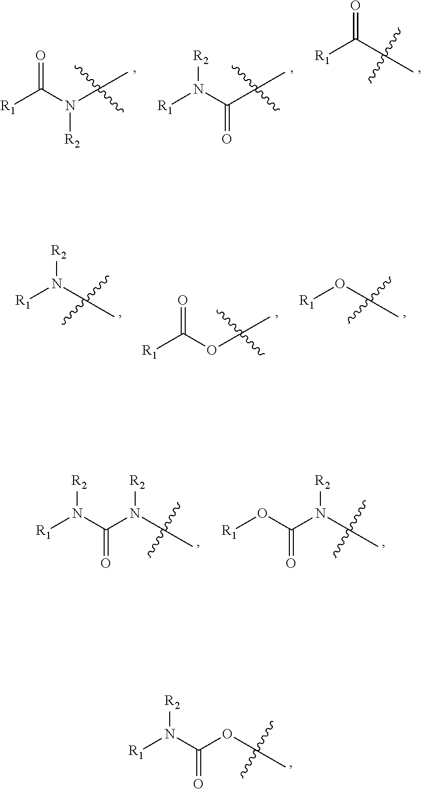

membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
and the definitions of other groups are consistent with those of embodiment nine.

For the compound of formula (I-2), the isomer thereof or the pharmaceutically acceptable salt thereof of the present disclosure, R₁₂ is selected from

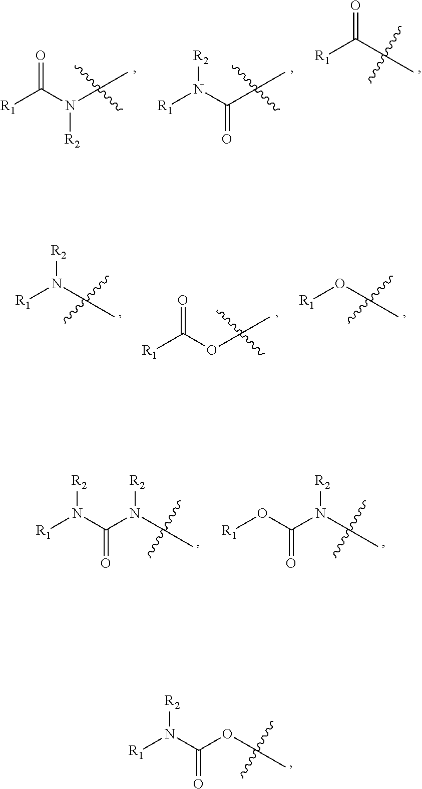

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₃ is selected from

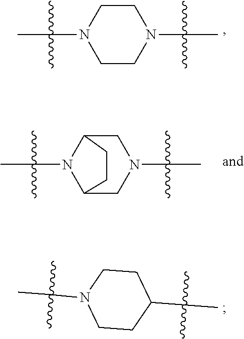 [SMILES: *N1CCN(*)CC1 ; *N1CC2CCC(C1)N2* ; *C1CCN(*)CC1]

Y₂ is selected from F and

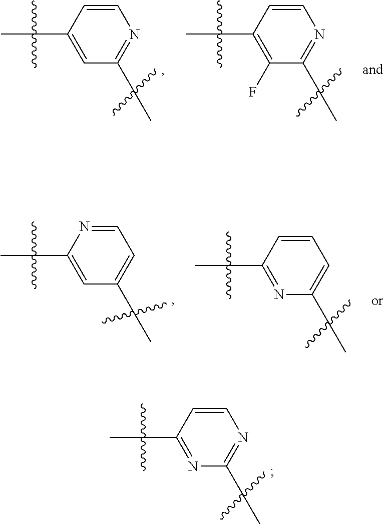 [SMILES: *c1ccnc(*)c1 ; *c1ccnc(*)c1F ; *c1ccnc(*)c1 ; *c1cccc(*)n1 ; *c1ccnc(*)n1]

L₁ is selected from a bond,

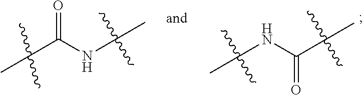 [SMILES: *NC(*)=O ; *NC(*)=O]

Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl.

For the compound of formula (I-2), the isomer thereof or the pharmaceutically acceptable salt thereof of the present disclosure,

R₁₂ is selected from

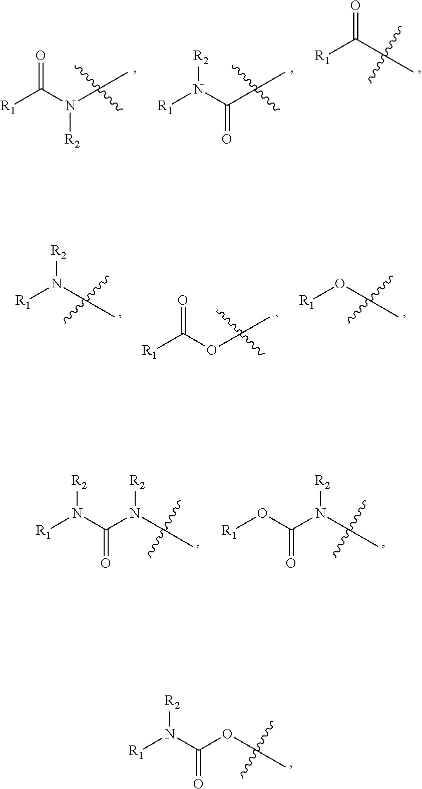

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₃ is selected from

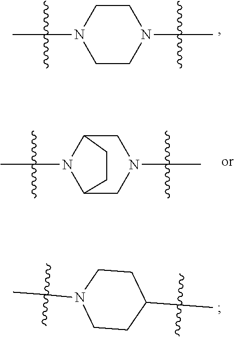 [SMILES: *N1CCN(*)CC1 ; *N1CC2CCC(C1)N2* ; *C1CCN(*)CC1]

Y₂ is selected from

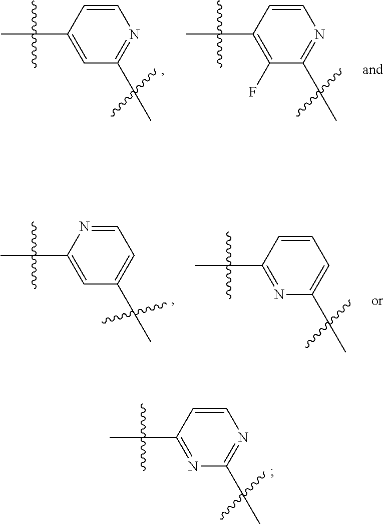 [SMILES: *c1ccnc(*)c1 ; *c1ccnc(*)c1F ; *c1ccnc(*)c1 ; *c1cccc(*)n1 ; *c1ccnc(*)n1]

L₁ is selected from a bond,

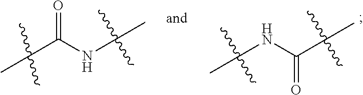 [SMILES: *NC(*)=O ; *NC(*)=O]

Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl.

For the compound of formula (I-2), the isomer thereof or the pharmaceutically acceptable salt thereof of the present disclosure,

R₁₂ is selected from

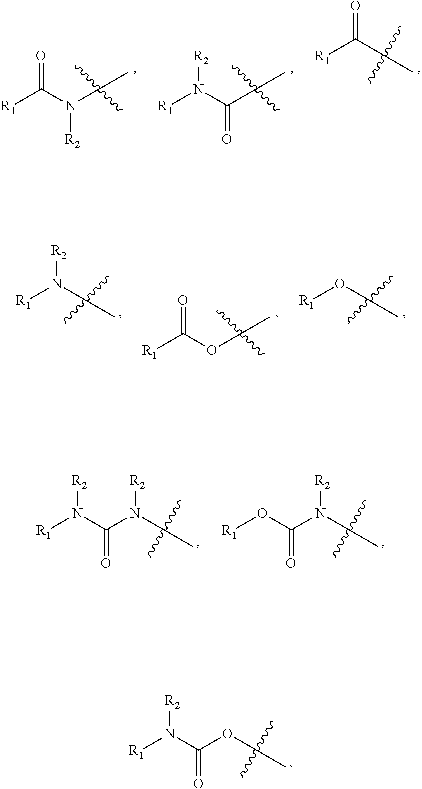

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₃ is selected from

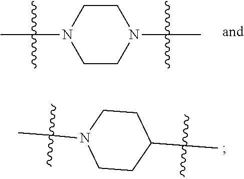 [SMILES: *N1CCN(*)CC1 ; *C1CCN(*)CC1]

Y₂ is selected from

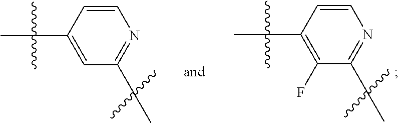 [SMILES: *c1ccnc(*)c1 ; *c1ccnc(*)c1F]

in certain embodiments, Y₂ is selected from

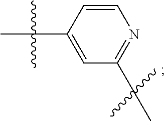 [SMILES: *c1ccnc(*)c1]

L₁ is selected from a bond;
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl.

For the compound of formula (I-2), the isomer thereof or the pharmaceutically acceptable salt thereof of the present disclosure,

R₁₂ is selected from

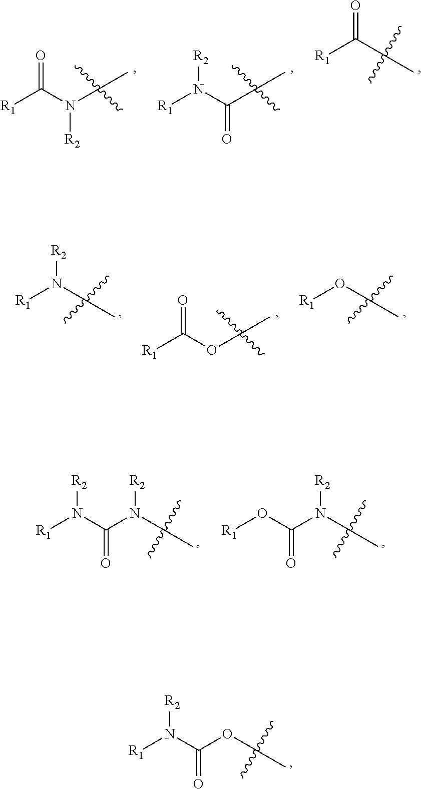

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₃ is selected from

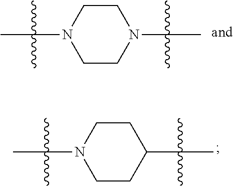 [SMILES: *N1CCN(*)CC1 ; *C1CCN(*)CC1]

Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
and Y₂ and L₁ are consistent with those of embodiment nine of the present disclosure.

For the compound of formula (I-2), the isomer thereof or the pharmaceutically acceptable salt thereof of the present disclosure,

R₁₂ is selected from

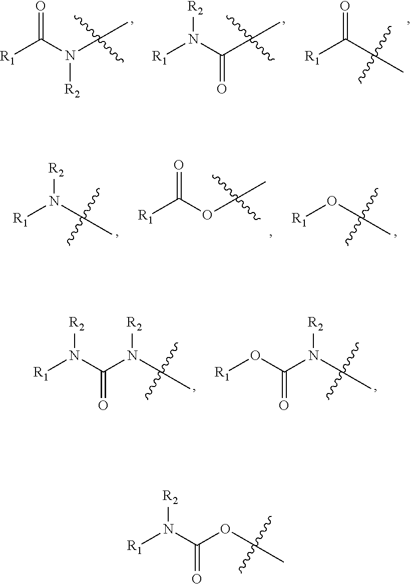

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₂ is selected from

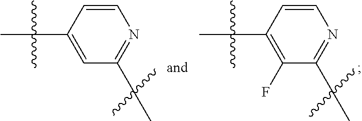 [SMILES: *c1ccnc(*)c1 ; *c1ccnc(*)c1F]

in certain embodiments, Y₂ is selected from

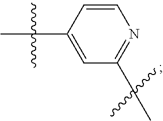 [SMILES: *c1ccnc(*)c1]

Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
and L₁ and Y₃ are consistent with those of embodiment nine of the present disclosure.

For the compound of formula (I-2), the isomer thereof or the pharmaceutically acceptable salt thereof of the present disclosure, R₁₂ is selected from

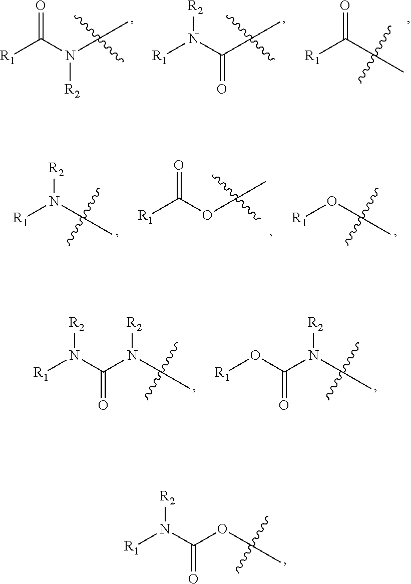

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
L₁ is selected from a bond;
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
and Y₂ and Y₃ are consistent with those of embodiment nine of the present disclosure.

For the compound of formula (I-2), the isomer thereof or the pharmaceutically acceptable salt thereof of the present disclosure,

R₁₂ is selected from

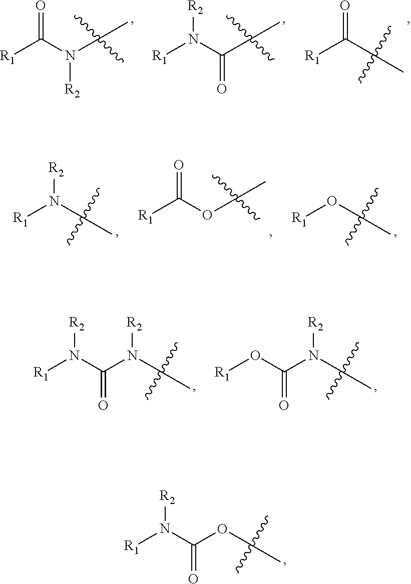

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₁ is selected from:

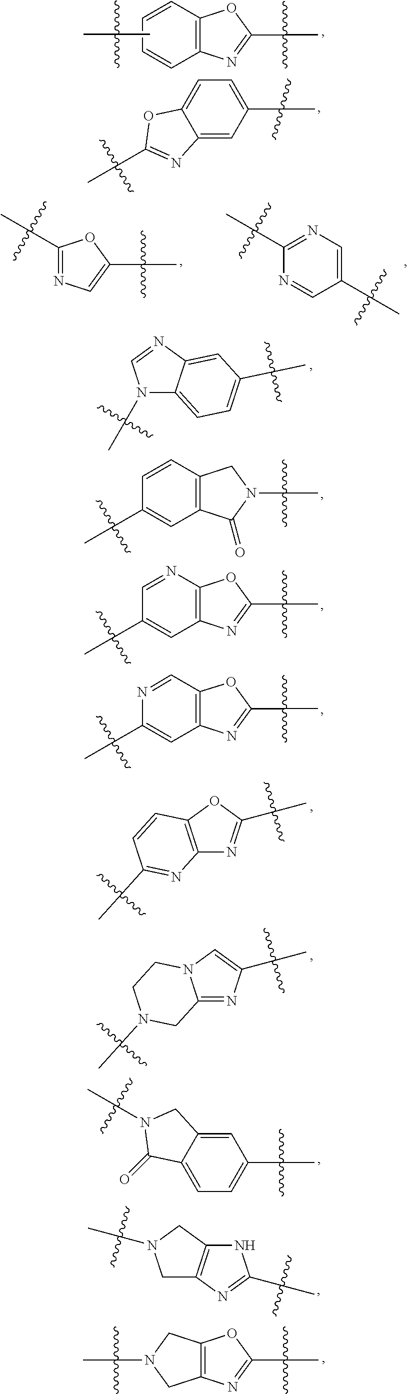

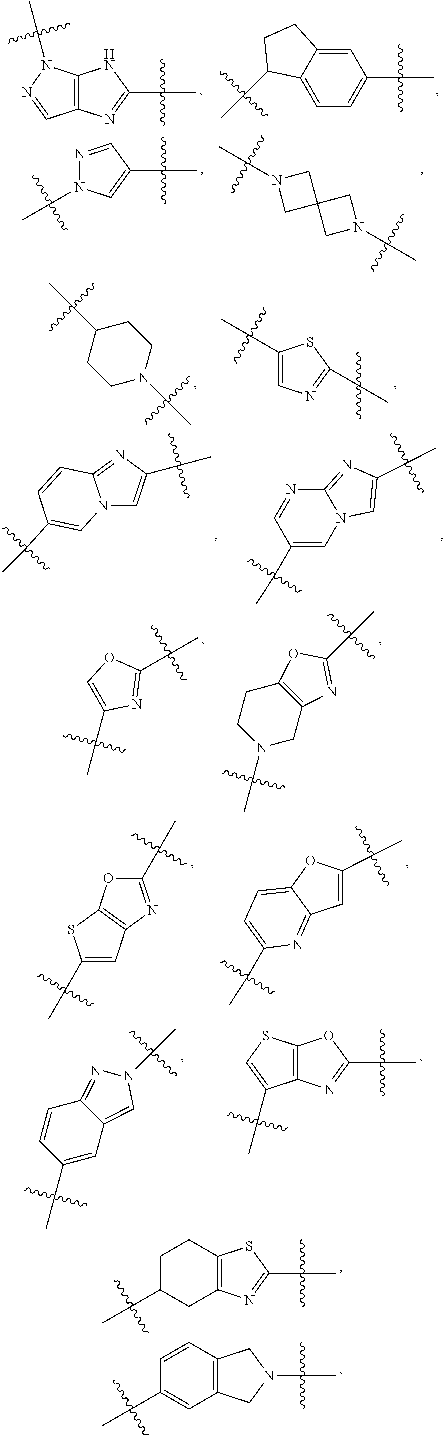

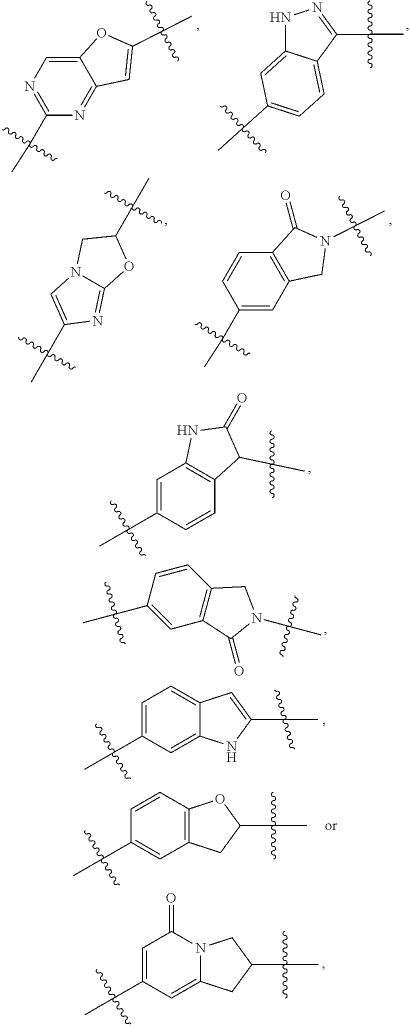

wherein the above-mentioned groups are optionally substituted with 0-3 R^{y1}; or
R^{y1} and R^{y2} together with L₁ form the following structure:

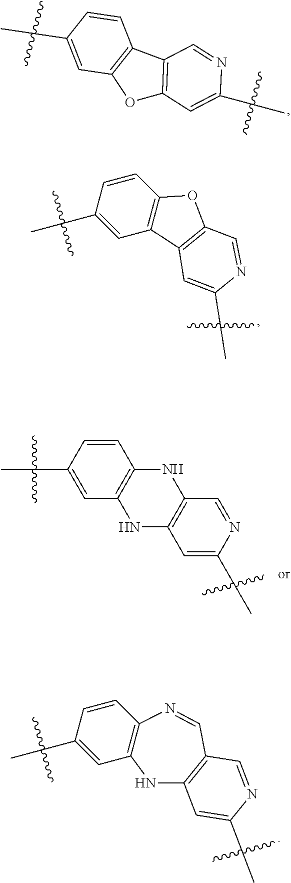 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

For the compound of formula (I-2), the isomer thereof or the pharmaceutically acceptable salt thereof of the present disclosure,

R₁₂ is selected from

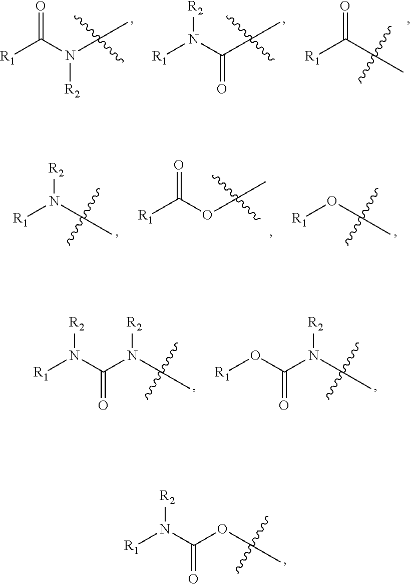

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₁ is selected from

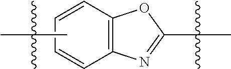

and optionally substituted with 0-3 R^{y1}; in certain embodiments, Y₁ is selected from

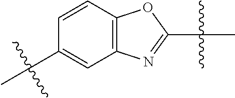 [SMILES: *c1ccc2oc(*)nc2c1]

and optionally substituted with 0-3 R^{y1};
R^{y1} is selected from F, methyl, difluoromethyl or trifluoromethyl;
and the definitions of other groups are consistent with those of embodiment nine.
For the compound of formula (I-2), the isomer thereof or the pharmaceutically acceptable salt thereof of the present disclosure,
R₁₂ is selected from

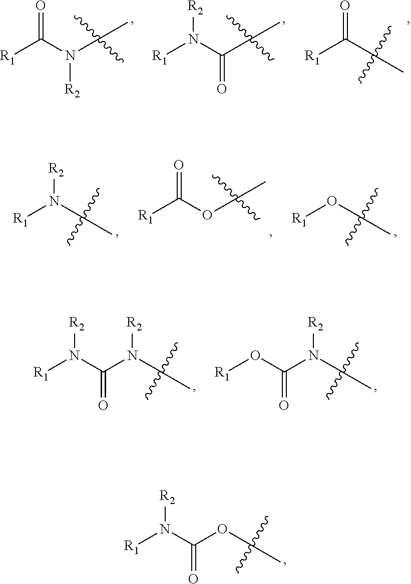

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
Y₁ is selected from

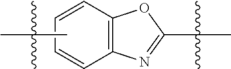

and optionally substituted with 0-3 R^{y1}; in certain embodiments, Y₁ is selected from

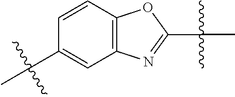 [SMILES: *c1ccc2oc(*)nc2c1]

and optionally substituted with 0-3 R^{y1};
R^{y1} is selected from F, methyl, difluoromethyl or trifluoromethyl;
Y₃ is selected from

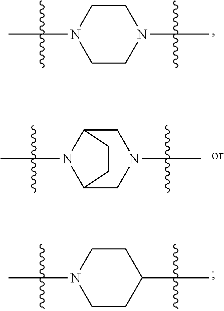 [SMILES: *N1CCN(*)CC1 ; *N1CC2CCC(C1)N2* ; *C1CCN(*)CC1]

Y₂ is selected from

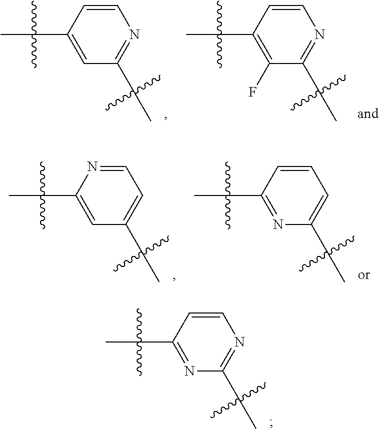 [SMILES: *c1ccnc(*)c1 ; *c1ccnc(*)c1F ; *c1ccnc(*)c1 ; *c1cccc(*)n1 ; *c1ccnc(*)n1]

and L₁ is selected from a bond

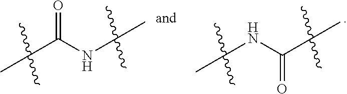 [SMILES: *NC(*)=O ; *NC(*)=O]

For the compound of formula (I-2), the isomer thereof or the pharmaceutically acceptable salt thereof of the present disclosure,

R₁₂ is selected from

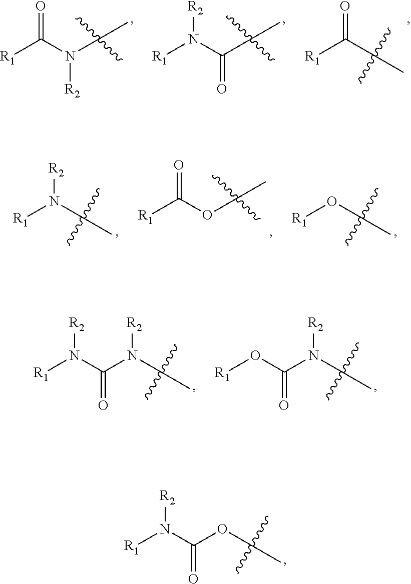

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
each R₂ is independently selected from: H or C₁₋₈ alkyl;

Y₁ is selected from

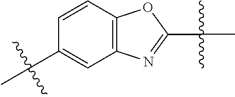 [SMILES: *c1ccc2oc(*)nc2c1]

and optionally substituted with 0-3 R^{y1};
R^{y1} is selected from F, methyl, difluoromethyl or trifluoromethyl;
Y₃ is selected from

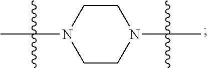 [SMILES: *N1CCN(*)CC1]

Y₂ is selected from

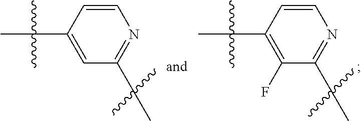 [SMILES: *c1ccnc(*)c1 ; *c1ccnc(*)c1F]

and L₁ is selected from a bond.

For the compound of formula (I-2), the isomer thereof or the pharmaceutically acceptable salt thereof of the present disclosure, R₁₂ is selected from pyridyl, thiazolyl, pyrazinyl, acetamido,

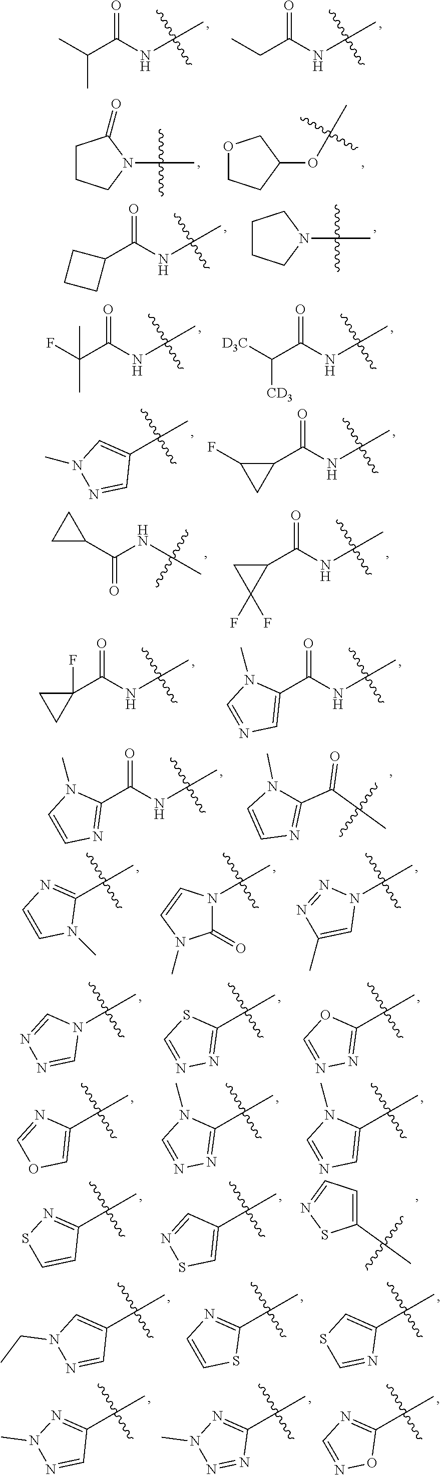

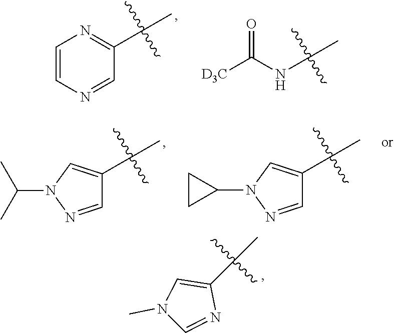

and the definitions of other groups are consistent with those of embodiment nine.

For the compound of formula (I-2), the isomer thereof or the pharmaceutically acceptable salt thereof of the present disclosure, R₁₂ is selected from pyridyl, thiazolyl, pyrazinyl, acetamido,

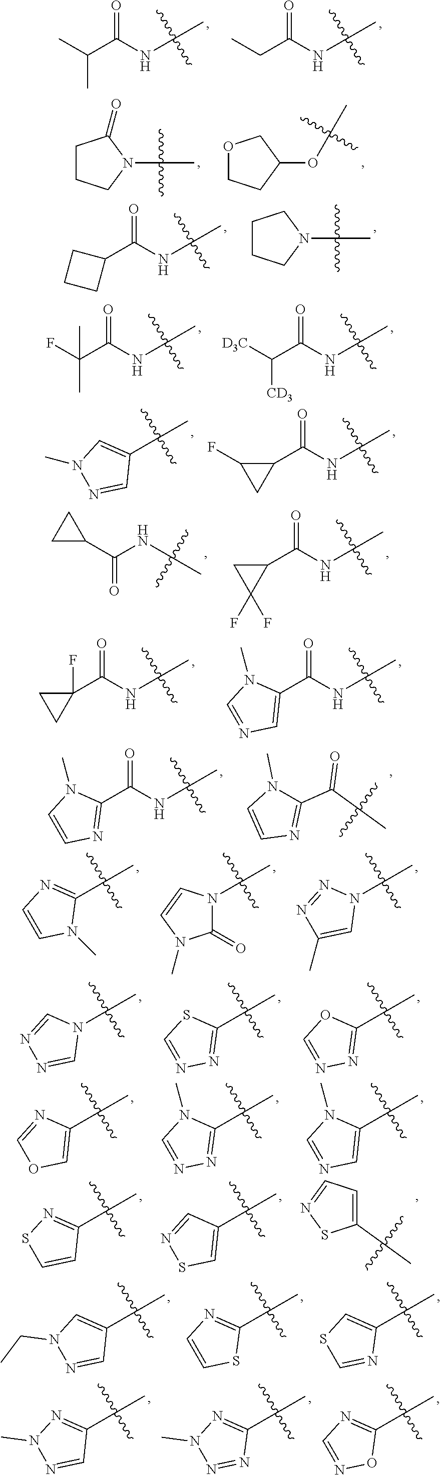

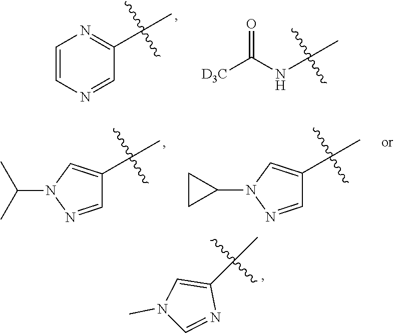

and the definitions of other groups are consistent with those of embodiment nine.

For the compound of formula (I-2), the isomer thereof or the pharmaceutically acceptable salt thereof of the present disclosure, R₁₂ is selected from pyridyl, thiazolyl, pyrazinyl, acetamido,

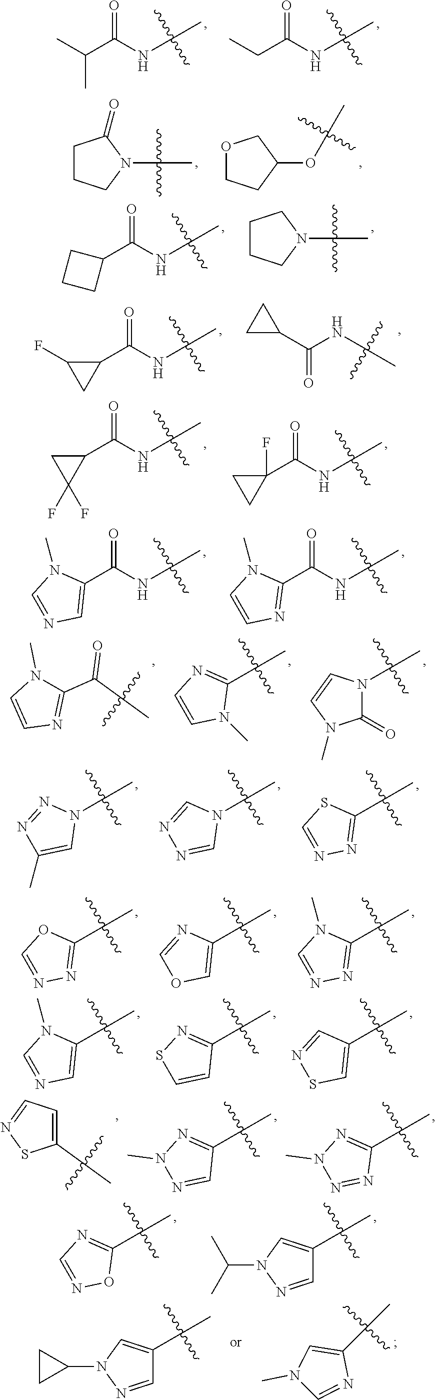

and the definitions of other groups are consistent with those of embodiment nine.

For the compound of formula (I-2), the isomer thereof or the pharmaceutically acceptable salt thereof of the present disclosure, R₁₂ is selected from pyridyl, thiazolyl, pyrazinyl,

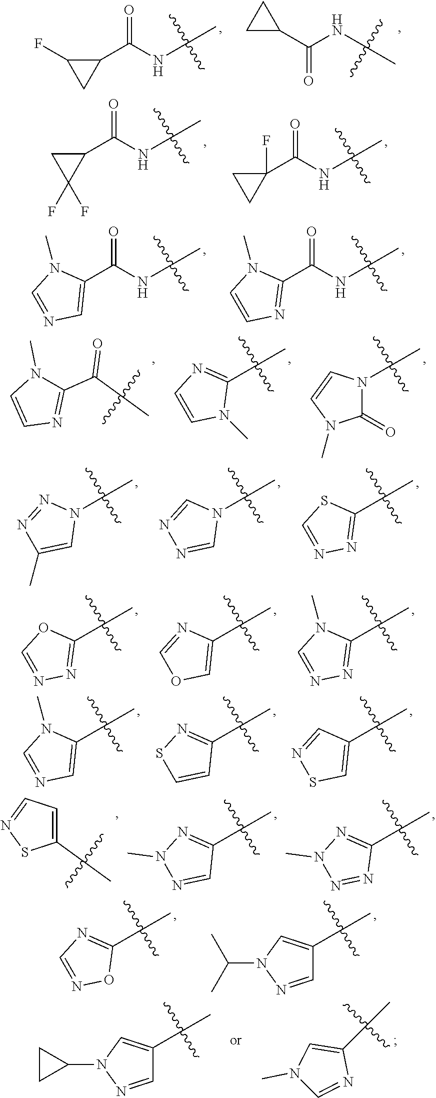

and the definitions of other groups are consistent with those of embodiment nine.

For the compound of formula (I-2), the isomer thereof or the pharmaceutically acceptable salt thereof of the present disclosure,

R₁₂ is selected from pyridyl, thiazolyl, pyrazinyl, acetamido,

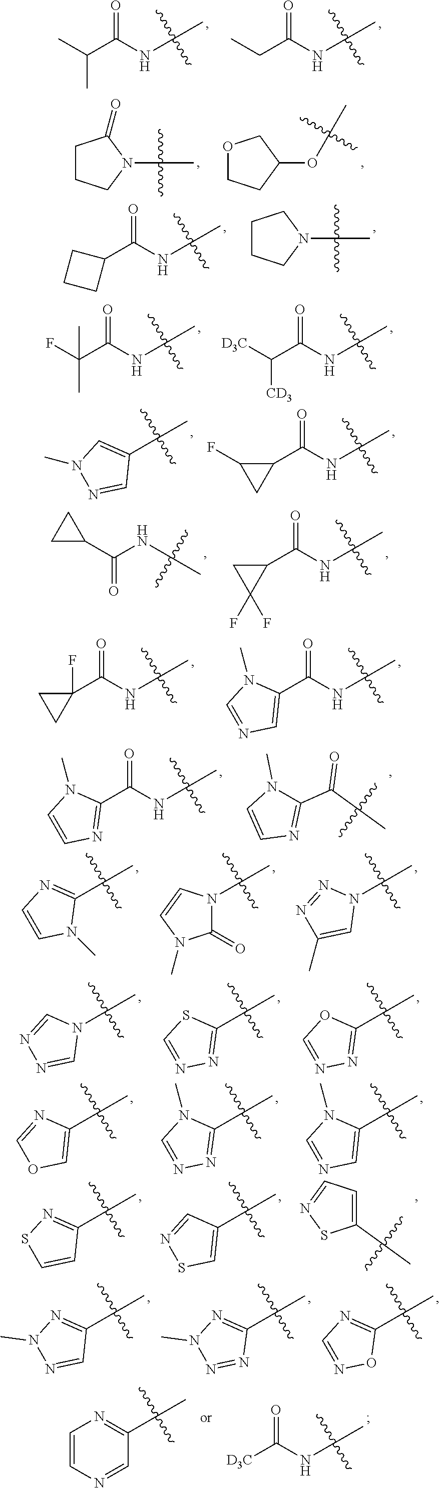

and the definitions of other groups are consistent with those of embodiment nine.

For the compound of formula (I-2), the isomer thereof or the pharmaceutically acceptable salt thereof of the present disclosure, R₁₂ is selected from pyridyl, thiazolyl, pyrazinyl, acetamido,

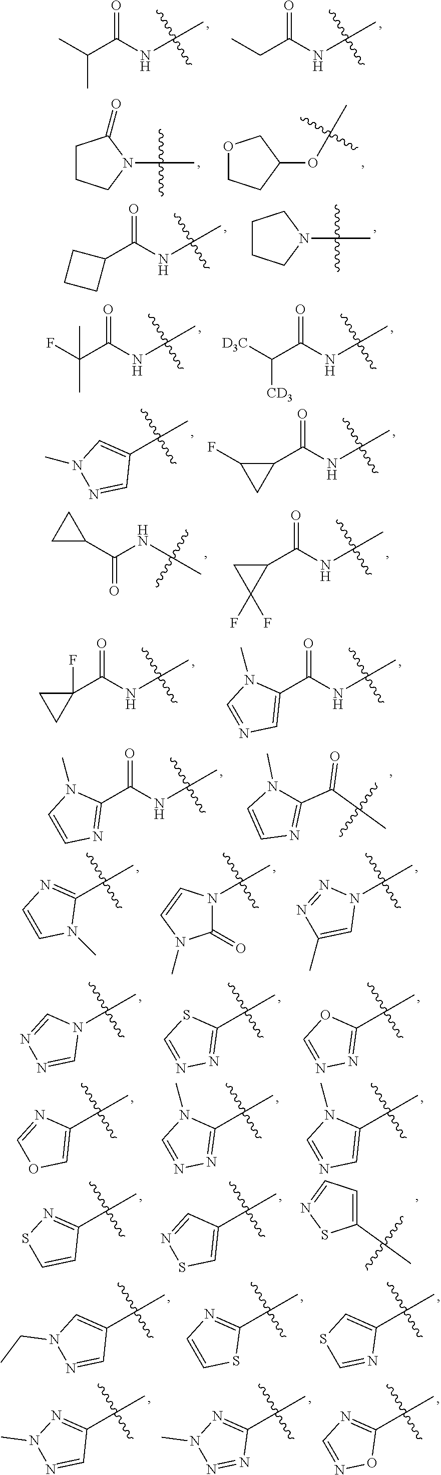

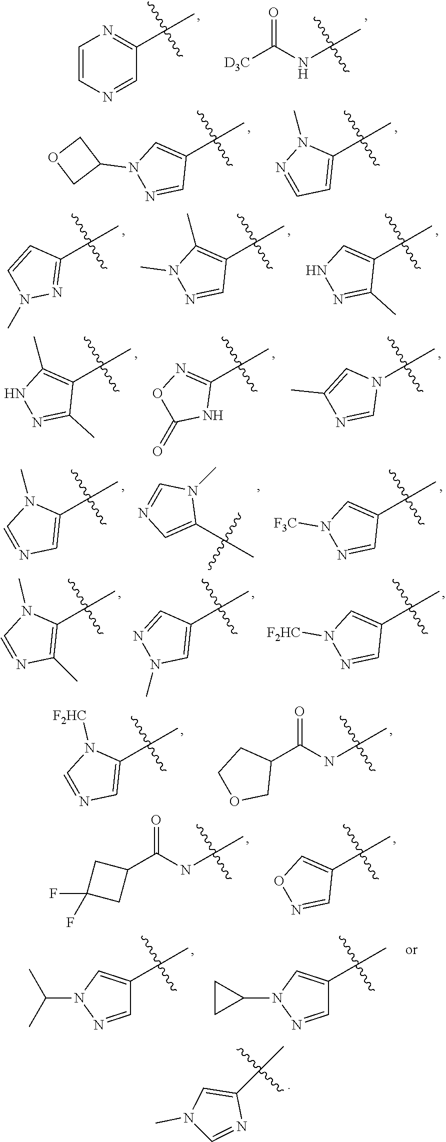

For the compound of formula (I-2), the isomer thereof or the pharmaceutically acceptable salt thereof of the present disclosure,

R₁₂ is selected from pyridyl, thiazolyl, pyrazinyl, acetamido,

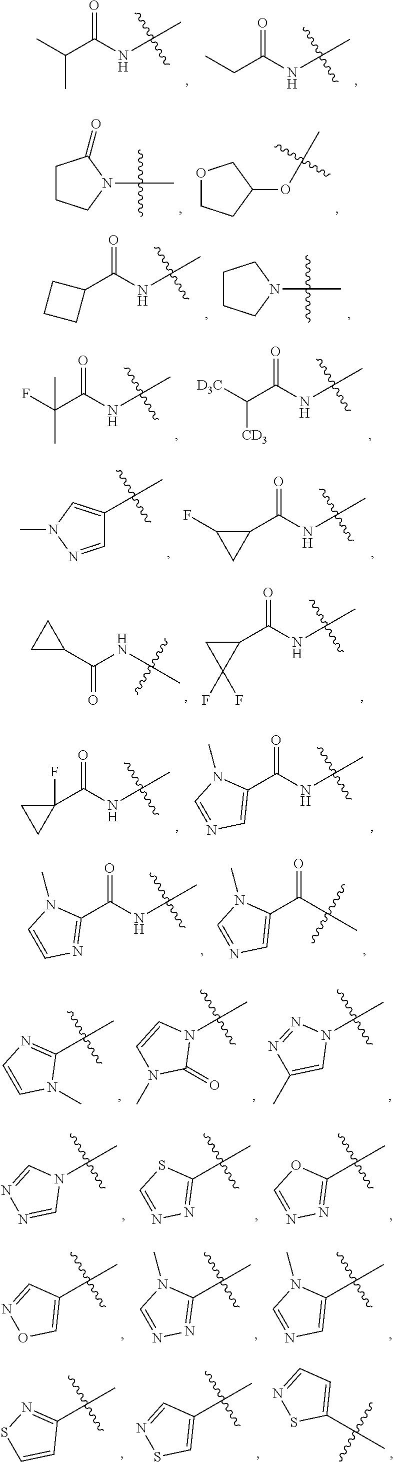

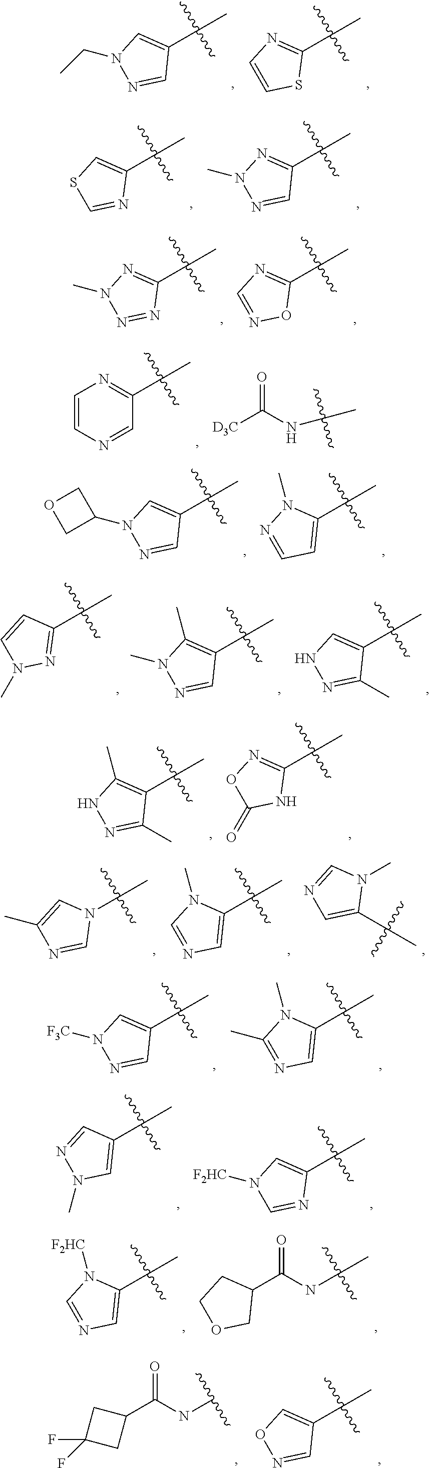

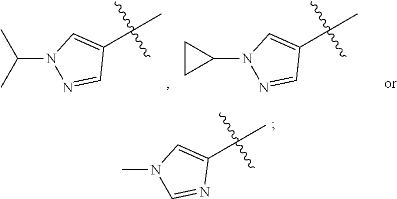

Y₃ is selected from

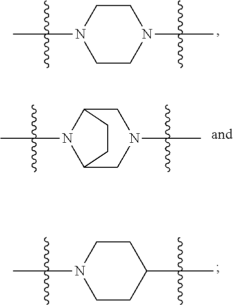 [SMILES: *N1CCN(*)CC1 ; *N1CC2CCC(C1)N2* ; *C1CCN(*)CC1]

Y₂ is selected from

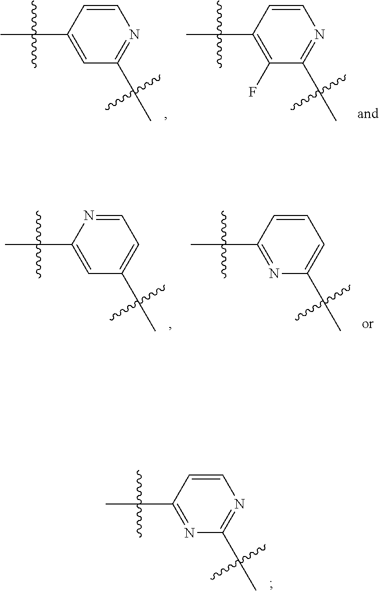 [SMILES: *c1ccnc(*)c1 ; *c1ccnc(*)c1F ; *c1ccnc(*)c1 ; *c1cccc(*)n1 ; *c1ccnc(*)n1]

L₁ is selected from a bond,

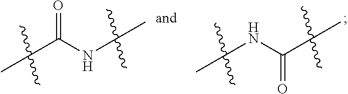 [SMILES: *NC(*)=O ; *NC(*)=O]

Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl.

For the compound of formula (I-2), the isomer thereof or the pharmaceutically acceptable salt thereof of the present disclosure,

R₁₂ is selected from pyridyl, thiazolyl, pyrazinyl, acetamido,

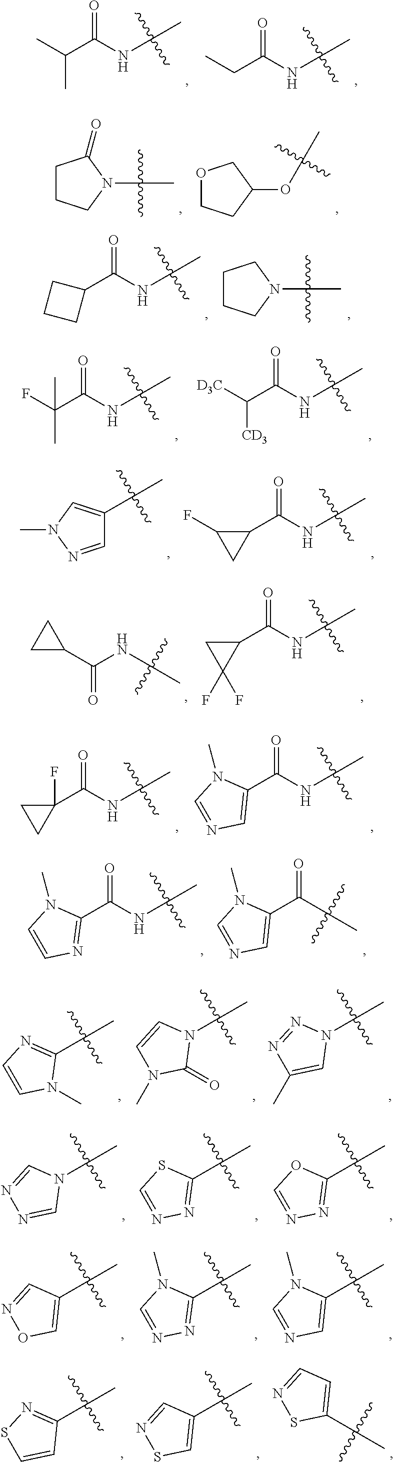

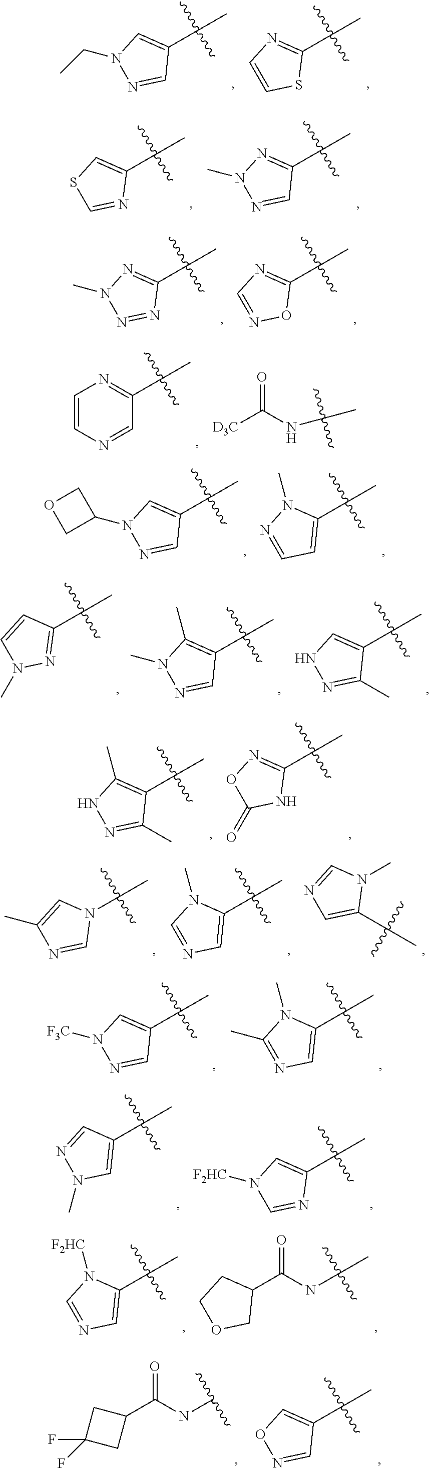

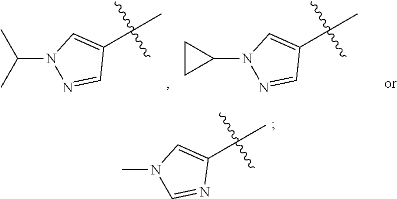

Y₃ is selected from

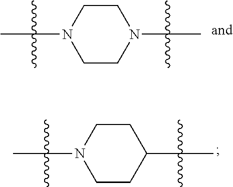 [SMILES: *N1CCN(*)CC1 ; *C1CCN(*)CC1]

Y₂ is selected from

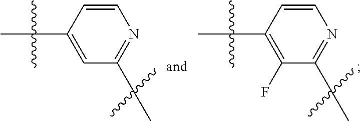 [SMILES: *c1ccnc(*)c1 ; *c1ccnc(*)c1F]

L₁ is selected from a bond;
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl.

For the compound of formula (I-2), the isomer thereof or the pharmaceutically acceptable salt thereof of the present disclosure,

R₁₂ is selected from pyridyl, thiazolyl, pyrazinyl, acetamido,

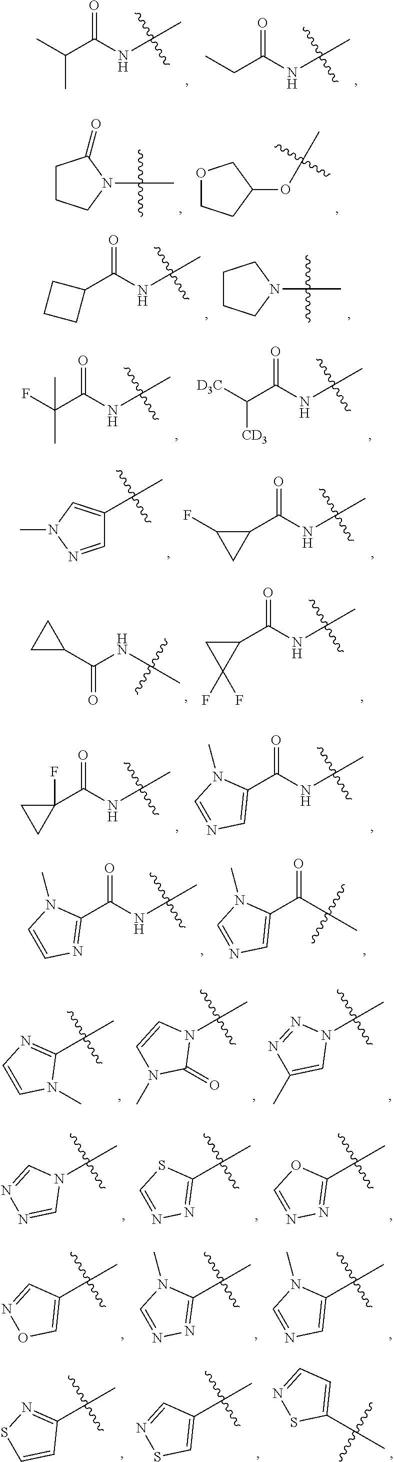

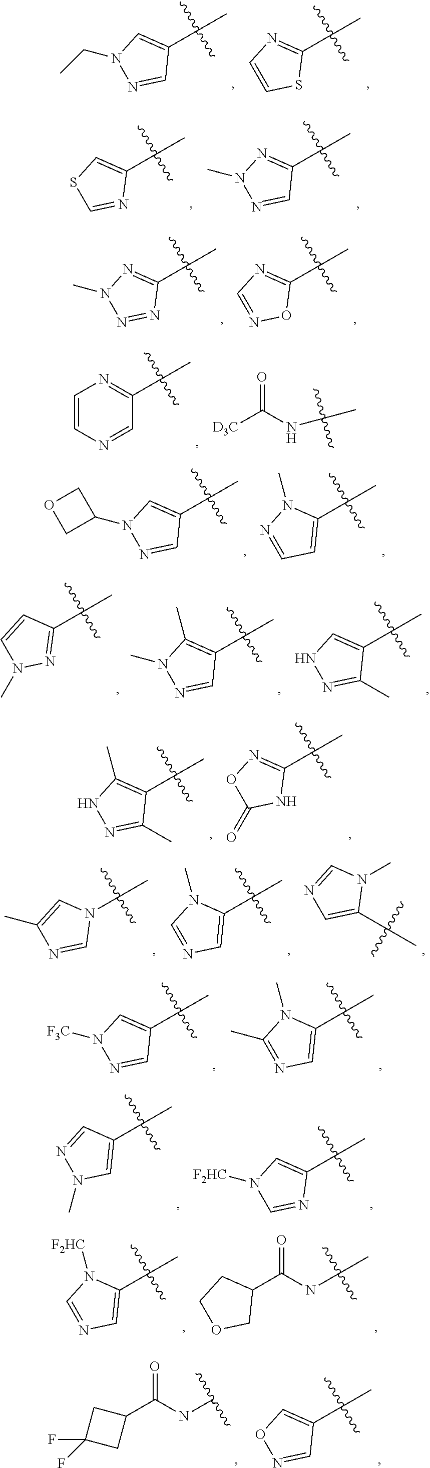

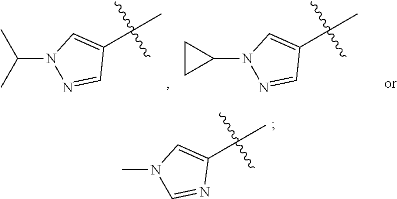

Y₃ is selected from

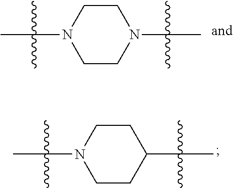 [SMILES: *N1CCN(*)CC1 ; *C1CCN(*)CC1]

Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
and the definitions of groups Y₂ and L₁ are consistent with those of embodiment nine of the present disclosure.

For the compound of formula (I-2), the isomer thereof or the pharmaceutically acceptable salt thereof of the present disclosure,

R₁₂ is selected from pyridyl, thiazolyl, pyrazinyl, acetamido,

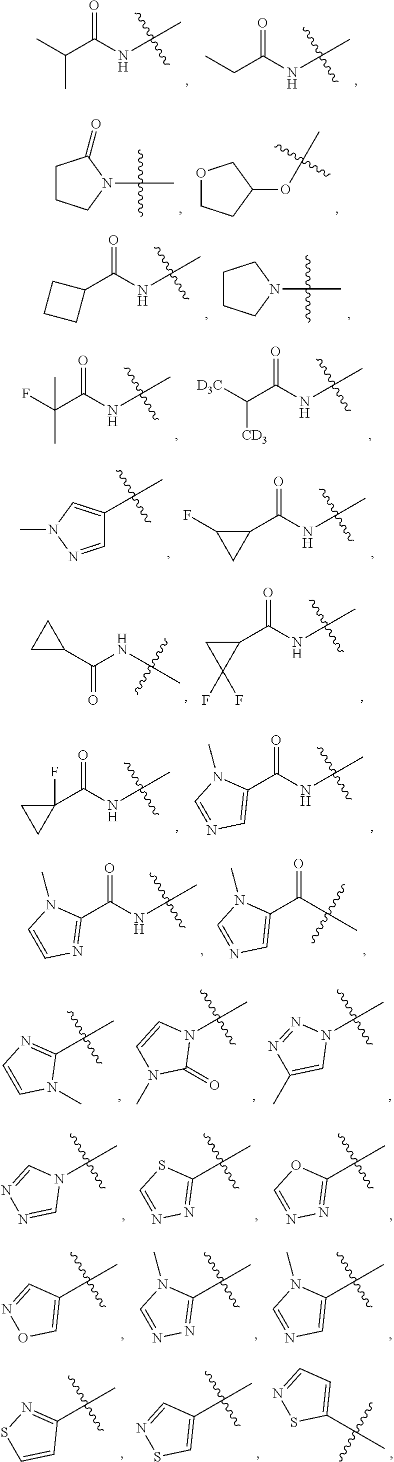

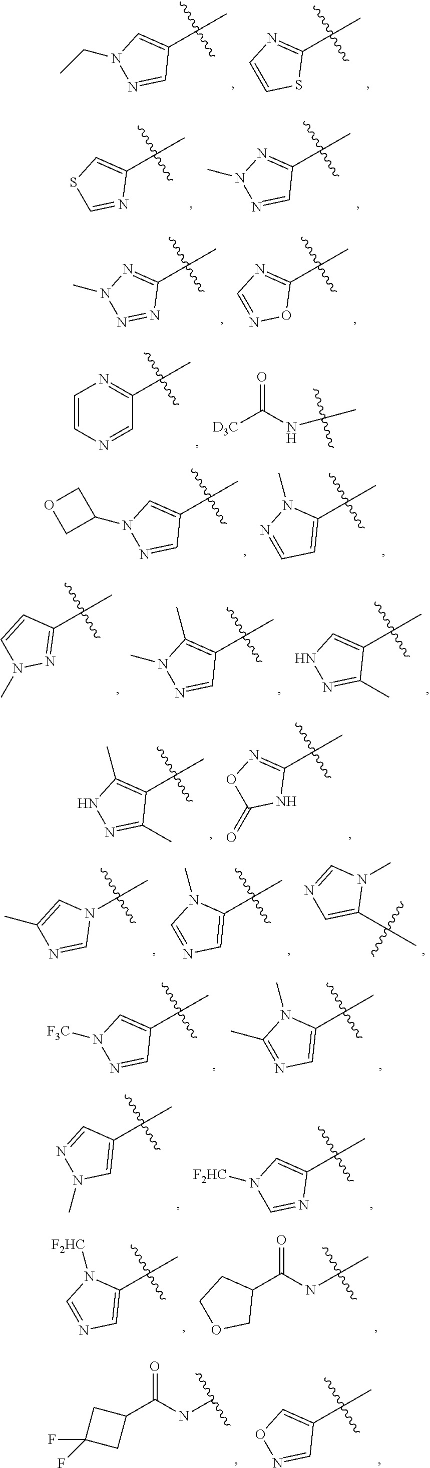

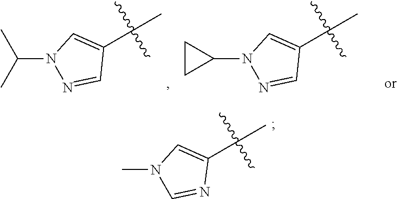

Y₂ is selected from

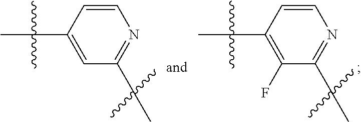 [SMILES: *c1ccnc(*)c1 ; *c1ccnc(*)c1F]

Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
and the definitions of groups Y₃ and L₁ are consistent with those of embodiment nine of the present disclosure.

For the compound of formula (I-2), the isomer thereof or the pharmaceutically acceptable salt thereof of the present disclosure,

R₁₂ is selected from pyridyl, thiazolyl, pyrazinyl, acetamido,

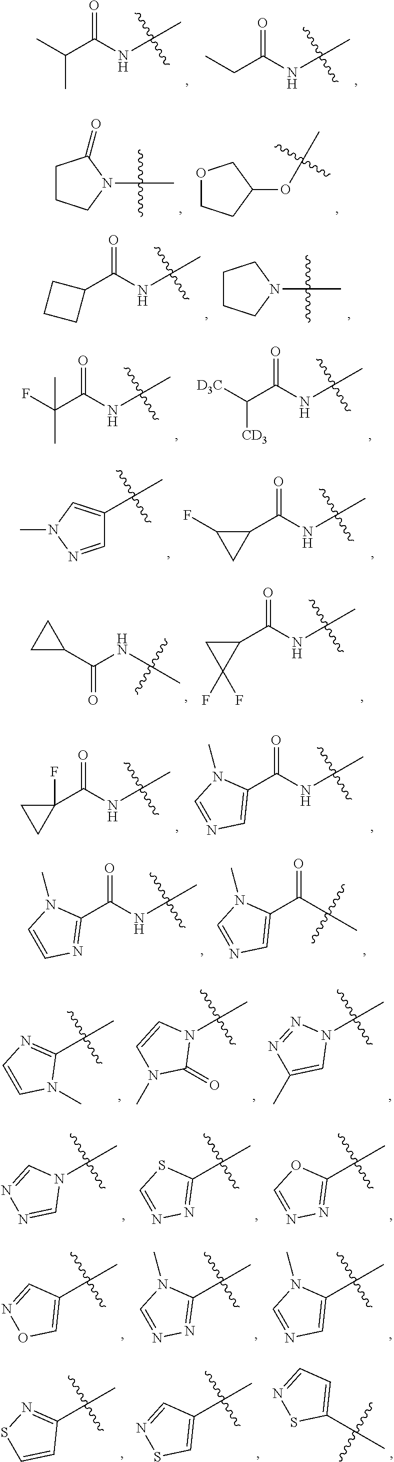

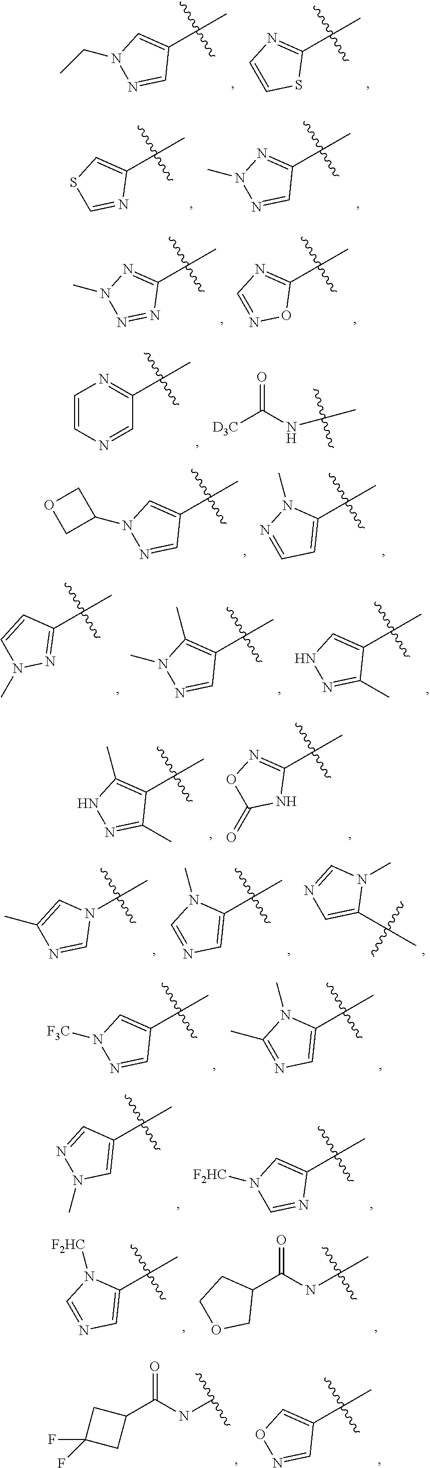

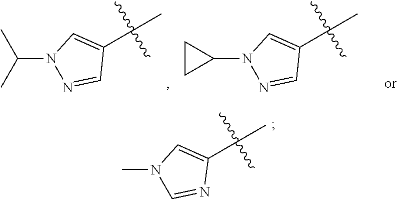

L₁ is selected from a bond;
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
and the definitions of groups Y₂ and Y₃ are consistent with those of embodiment nine of the present disclosure.

For the compound of formula (I-2), the isomer thereof or the pharmaceutically acceptable salt thereof of the present disclosure,

R₁₂ is selected from pyridyl, thiazolyl, pyrazinyl, acetamido,

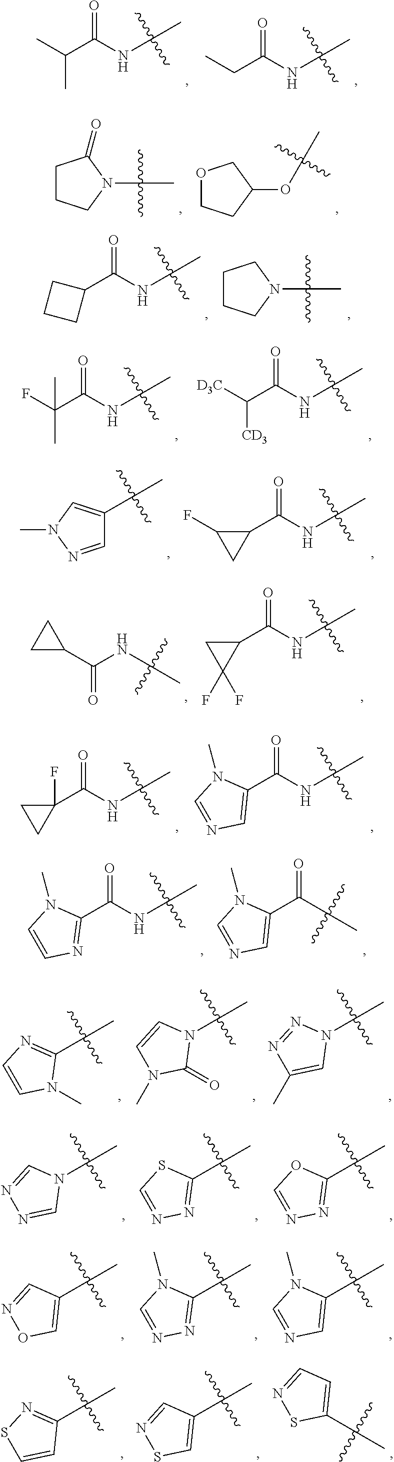

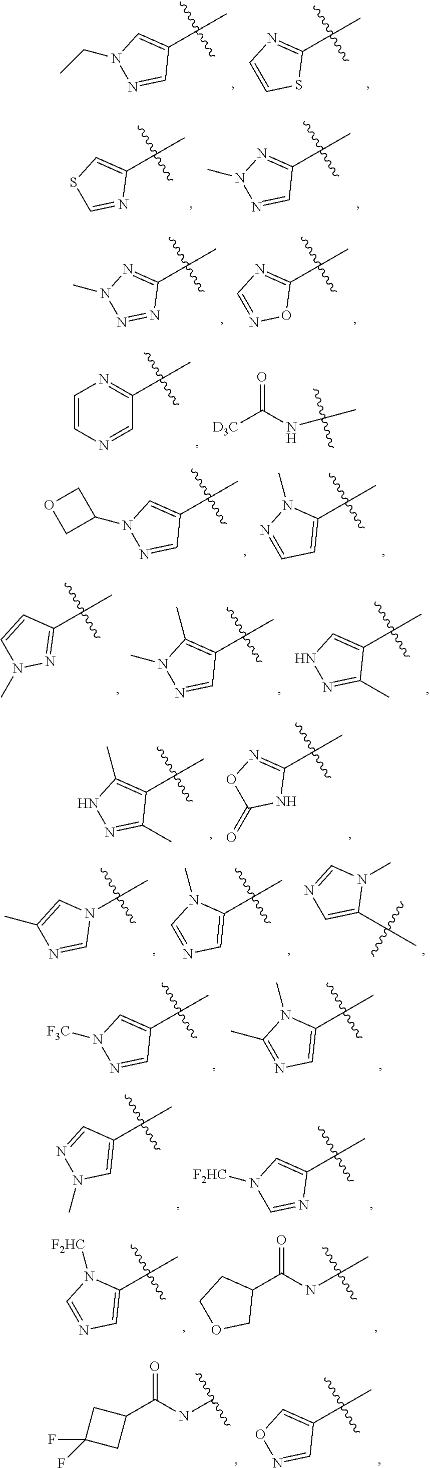

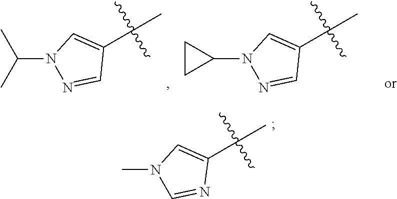

Y₁ is selected from

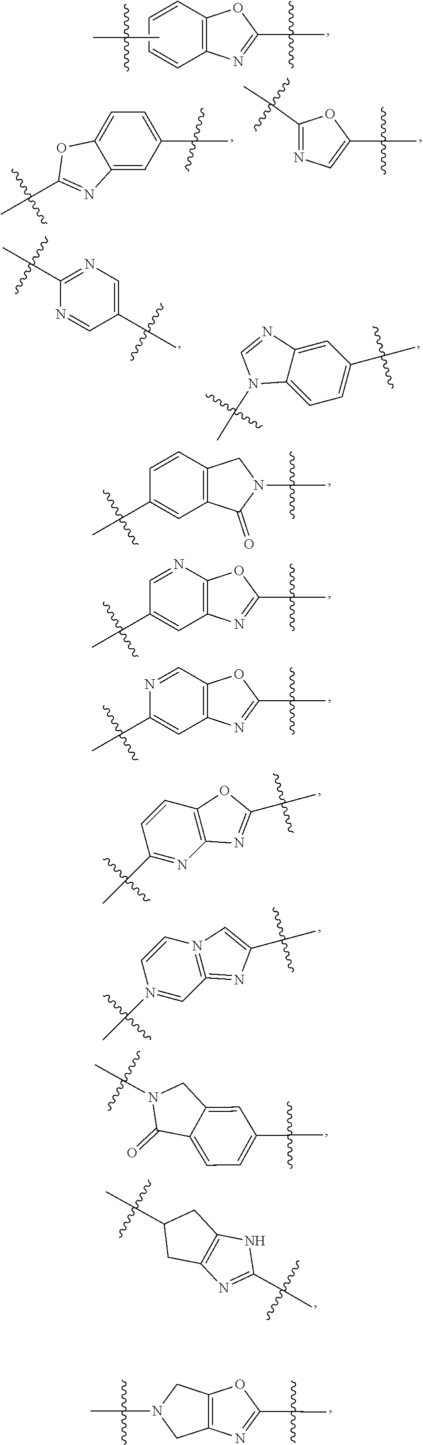

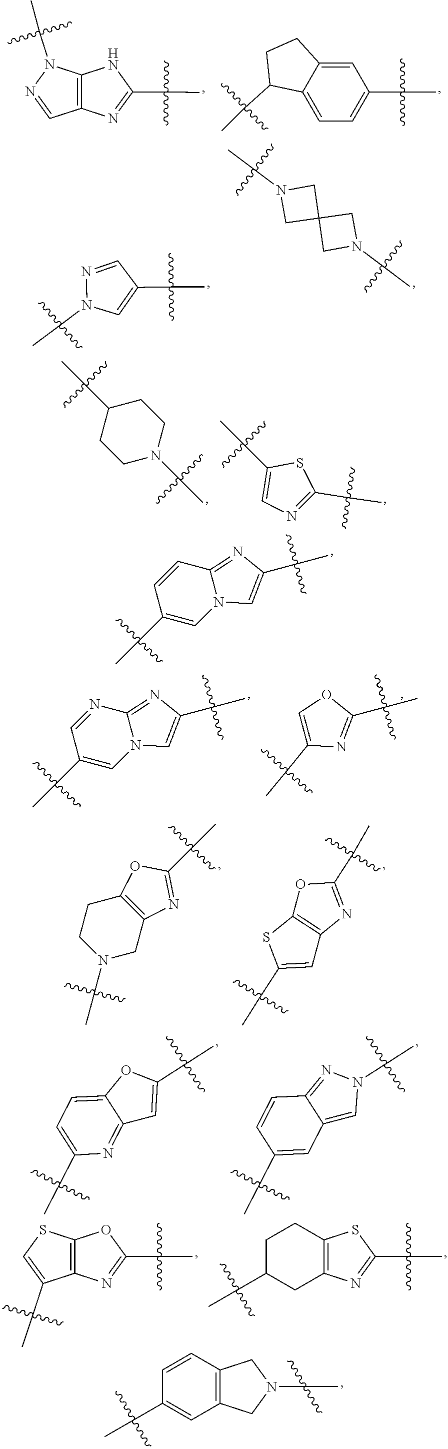

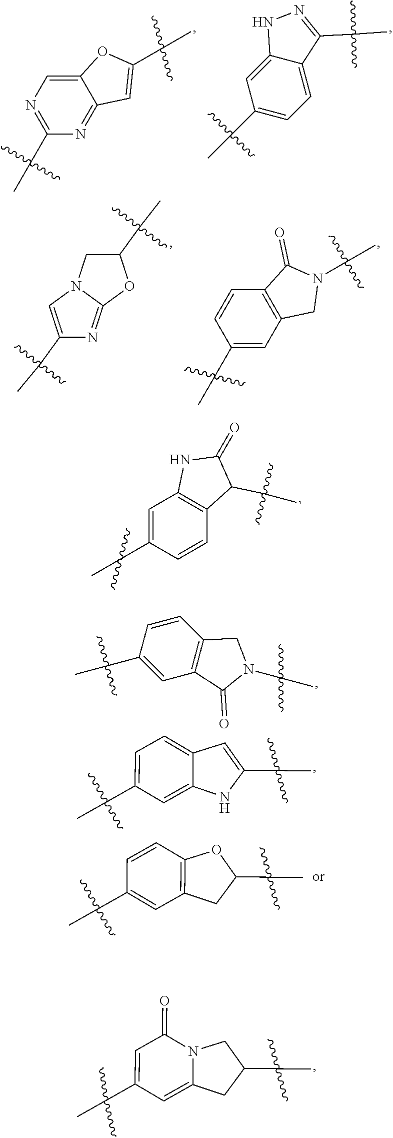

wherein the above-mentioned groups are optionally substituted with 0-3 R^{y1}; or
R^{y1} and R^{y2} together with L₁ form the following structure:

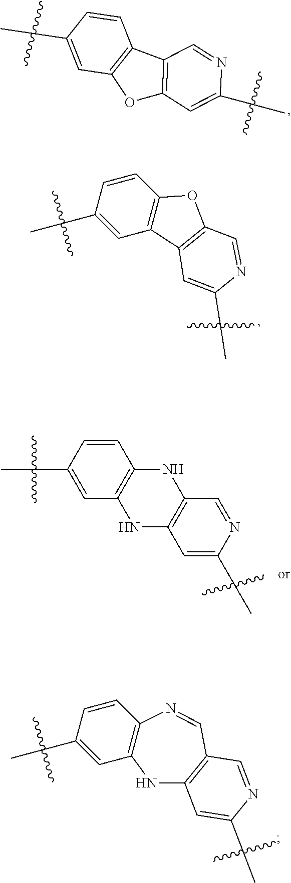 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

and the definitions of other groups are consistent with those of embodiment nine.

For the compound of formula (I-2), the isomer thereof or the pharmaceutically acceptable salt thereof of the present disclosure,

R₁₂ is selected from pyridyl, thiazolyl, pyrazinyl, acetamido,

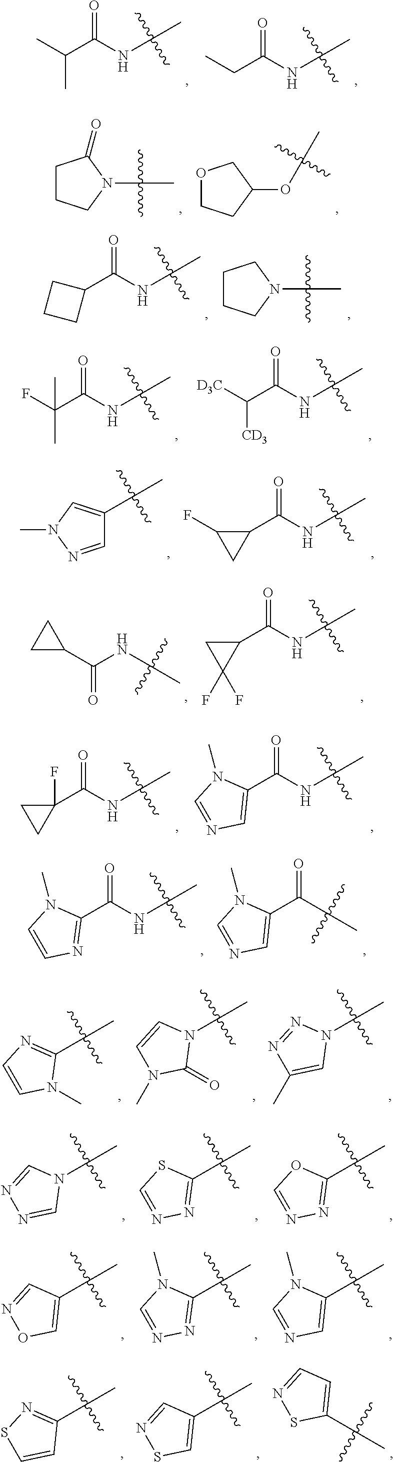

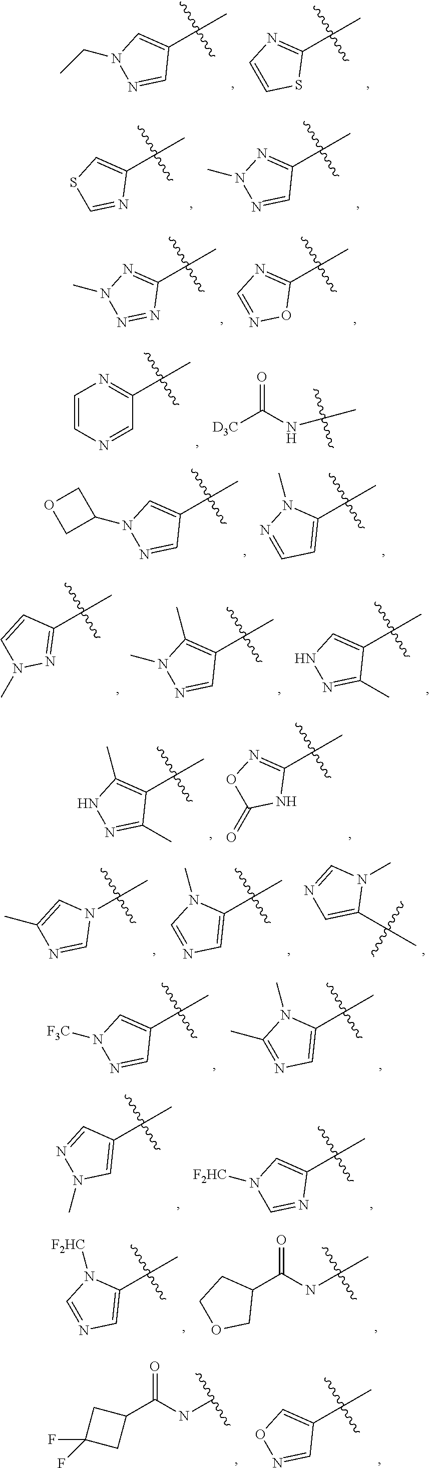

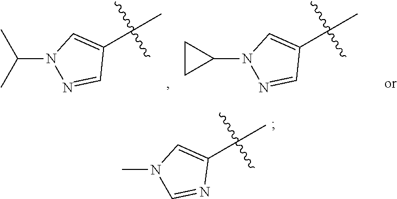

Y₁ is selected from

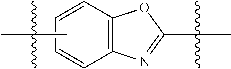

and optionally substituted with 0-3 R^{y1}; in certain embodiments, Y₁ is selected from

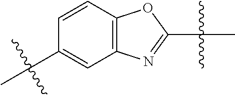 [SMILES: *c1ccc2oc(*)nc2c1]

and optionally substituted with 0-3 R^{y1};
R^{y1} is selected from F, methyl, difluoromethyl or trifluoromethyl;
and the definitions of other groups are consistent with those of embodiment nine.

For the compound of formula (I-2), the isomer thereof or the pharmaceutically acceptable salt thereof of the present disclosure,

R₁₂ is selected from pyridyl, thiazolyl, pyrazinyl, acetamido,

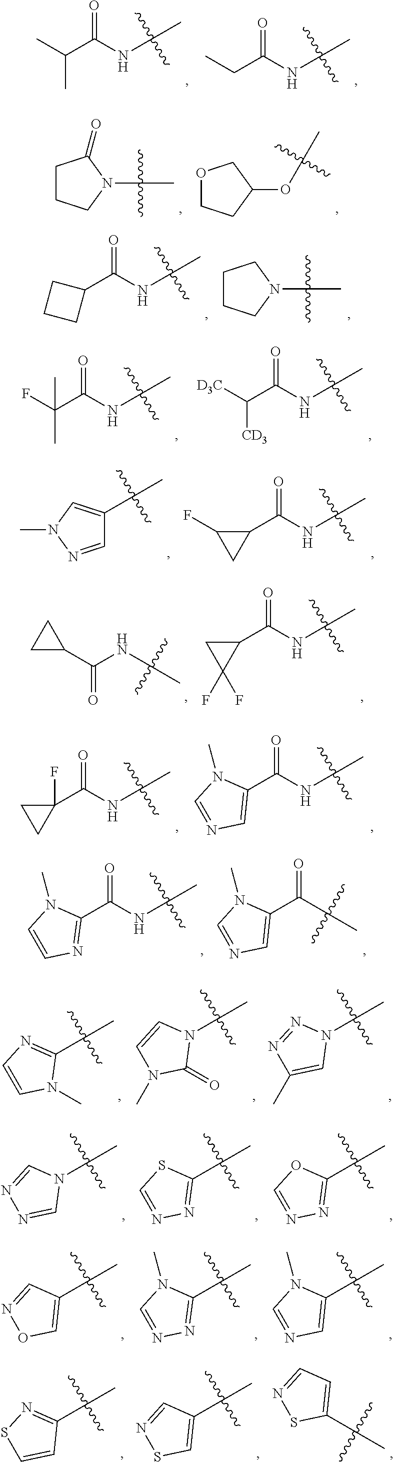

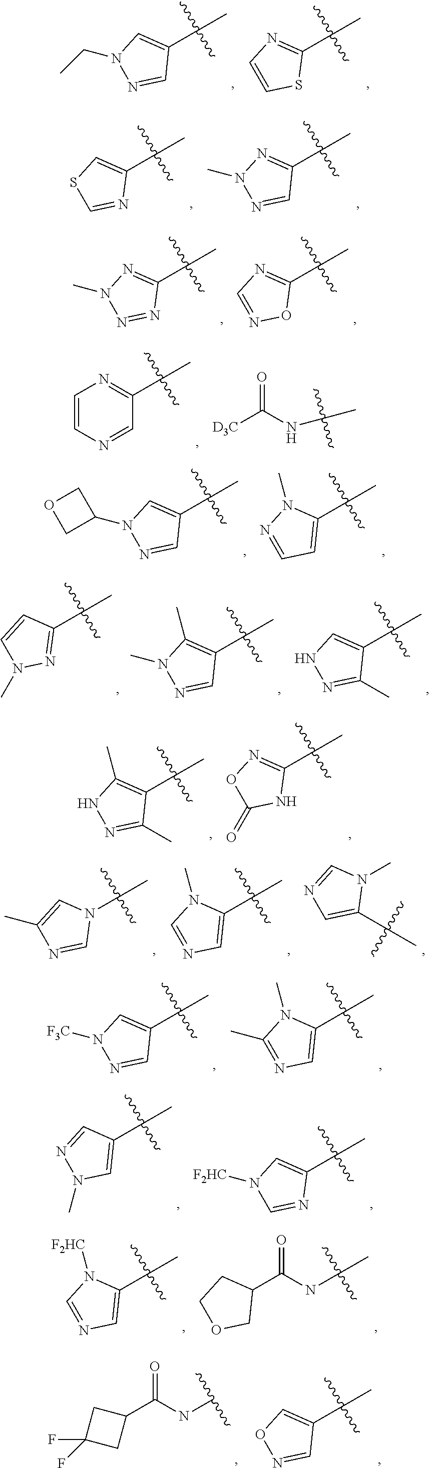

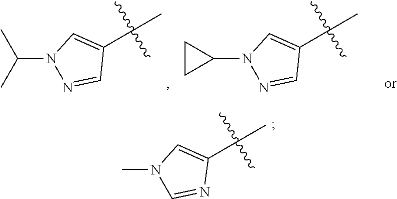

Y₁ is selected from

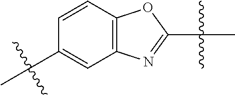 [SMILES: *c1ccc2oc(*)nc2c1]

and optionally substituted with 0-3 R^{y1};
R^{y1} is selected from F, methyl, difluoromethyl or trifluoromethyl;
Y₃ is selected from

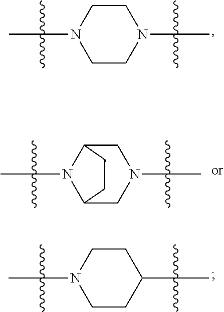 [SMILES: *N1CCN(*)CC1 ; *N1CC2CCC(C1)N2* ; *C1CCN(*)CC1]

Y₂ is selected from

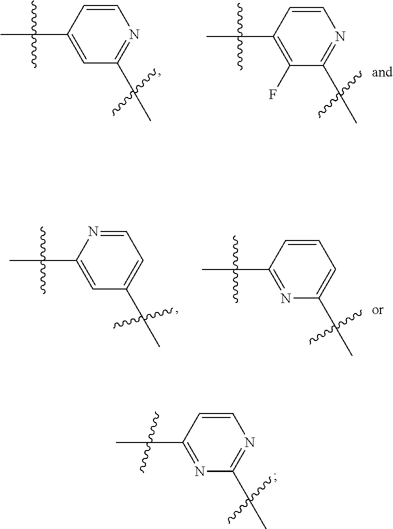 [SMILES: *c1ccnc(*)c1 ; *c1ccnc(*)c1F ; *c1ccnc(*)c1 ; *c1cccc(*)n1 ; *c1ccnc(*)n1]

and L₁ is selected from a bond,

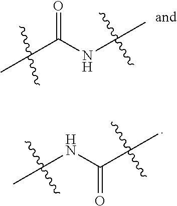 [SMILES: *NC(*)=O ; *NC(*)=O]

For the compound of formula (I-2), the isomer thereof or the pharmaceutically acceptable salt thereof of the present disclosure,

R₁₂ is selected from

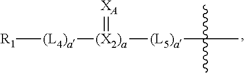

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₂ is selected from C;
L₄ and L₅ are each independently selected from: a bond, NR₂, CR₃R₄, O, or S;
X_{A} is selected from O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl,
C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
R₂, R₃ and R₄ are each independently selected from: H, halogen, C₁₋₈ alkyl or C₁₋₈ haloalkyl;
Y₁ is selected from

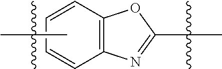

and optionally substituted with 0-3 R^{y1}; in certain embodiments, Y₁ is selected from

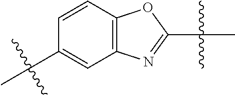 [SMILES: *c1ccc2oc(*)nc2c1]

and optionally substituted with 0-3 R^{y1};
R^{y1} is selected from F, methyl, difluoromethyl or trifluoromethyl;
Y₃ is selected from

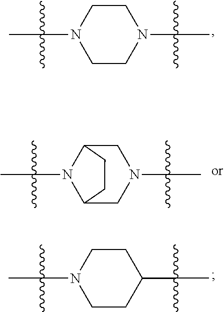 [SMILES: *N1CCN(*)CC1 ; *N1CC2CCC(C1)N2* ; *C1CCN(*)CC1]

Y₂ is selected from

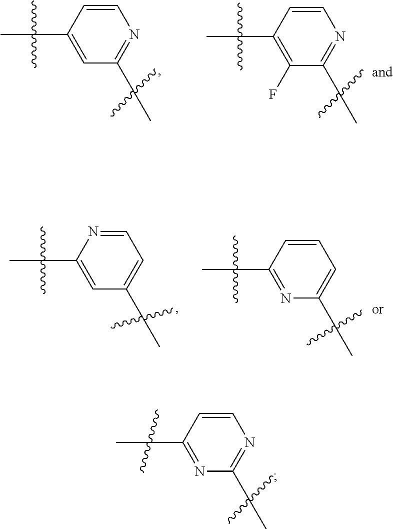 [SMILES: *c1ccnc(*)c1 ; *c1ccnc(*)c1F ; *c1ccnc(*)c1 ; *c1cccc(*)n1 ; *c1ccnc(*)n1]

and L₁ is selected from a bond,

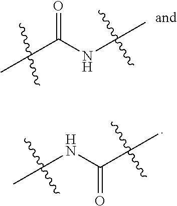 [SMILES: *NC(*)=O ; *NC(*)=O]

For the compound of formula (I-2), the isomer thereof or the pharmaceutically acceptable salt thereof of the present disclosure,

R₁₂ is selected from

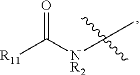

and in certain embodiments, R₁₂ is selected from

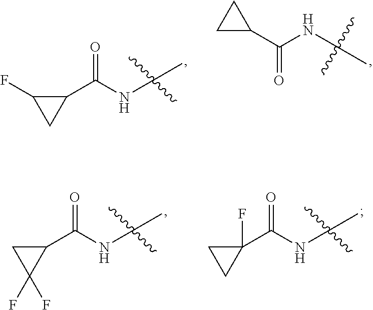 [SMILES: *NC(=O)C1CC1F ; *NC(=O)C1CC1 ; *NC(=O)C1CC1(F)F ; *NC(=O)C1(F)CC1]

in certain embodiments, R₁₂ is selected from

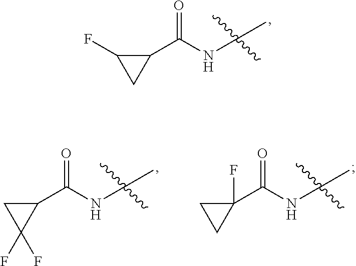 [SMILES: *NC(=O)C1CC1F ; *NC(=O)C1CC1(F)F ; *NC(=O)C1(F)CC1]

R₁ is selected from C₁₋₈ cycloalkyl, and in certain embodiments, R₁ is selected from cyclopropyl;
R₂ is selected from H or C₁₋₈ alkyl;
Y₁ is selected from

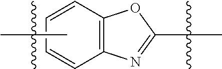

and optionally substituted with 0-3 R^{y1}; in certain embodiments, Y₁ is selected from

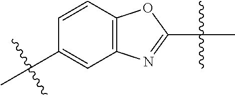 [SMILES: *c1ccc2oc(*)nc2c1]

and optionally substituted with 0-3 R^{y1};
R^{y1} is selected from F, methyl, difluoromethyl or trifluoromethyl;
Y₃ is selected from

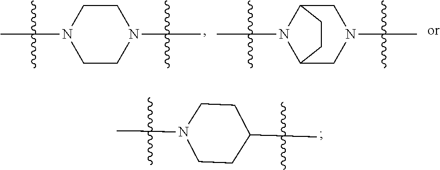 [SMILES: *N1CCN(*)CC1 ; *N1CC2CCC(C1)N2* ; *C1CCN(*)CC1]

Y₂ is selected from

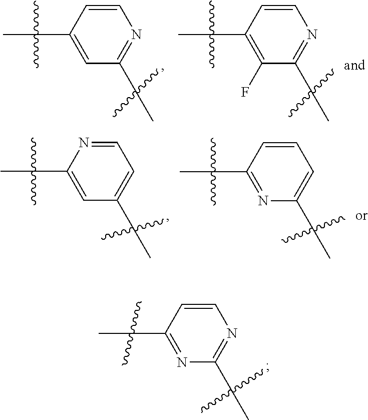 [SMILES: *c1ccnc(*)c1 ; *c1ccnc(*)c1F ; *c1ccnc(*)c1 ; *c1cccc(*)n1 ; *c1ccnc(*)n1]

and L₁ is selected from a bond,

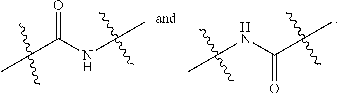 [SMILES: *NC(*)=O ; *NC(*)=O]

For the compound of formula (I-2), the isomer thereof or the pharmaceutically acceptable salt thereof of the present disclosure,

R₁₂ is selected from 5-10 membered heteroaryl, wherein the heteroaryl is optionally substituted with 0-3 of the following groups: C₁₋₈ alkyl and C₁₋₈ haloalkyl; in certain embodiments, R₁₂ is selected from

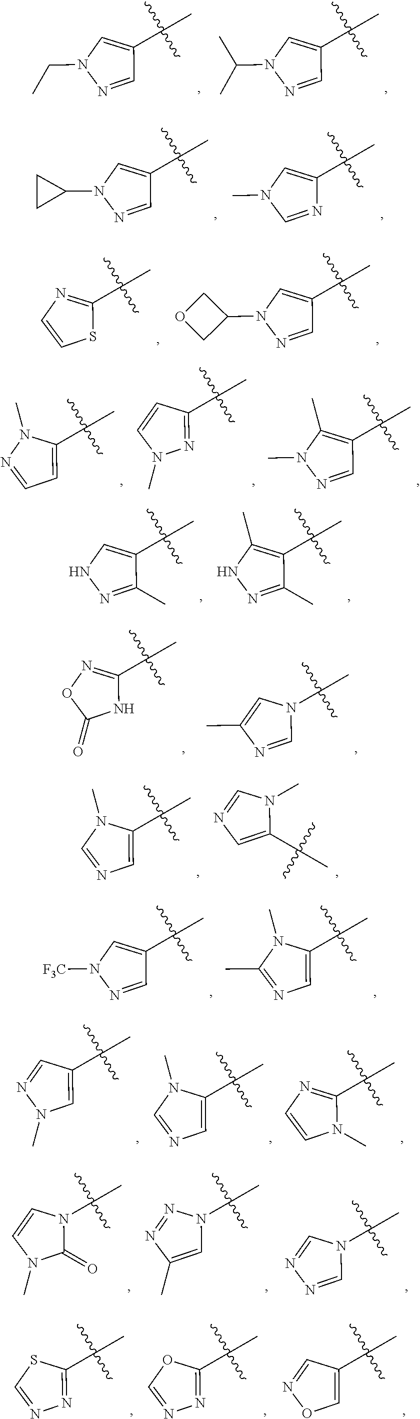

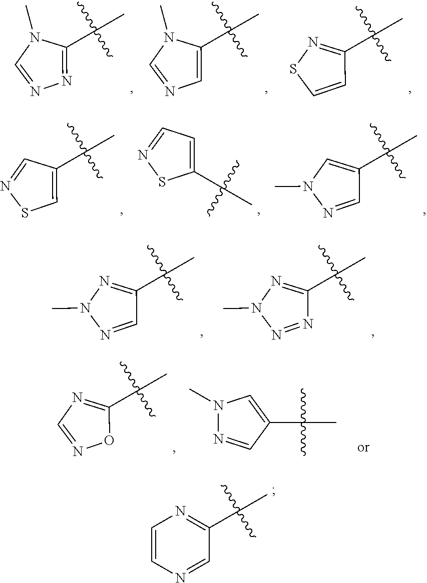

in certain embodiments, R₁₂ is selected from

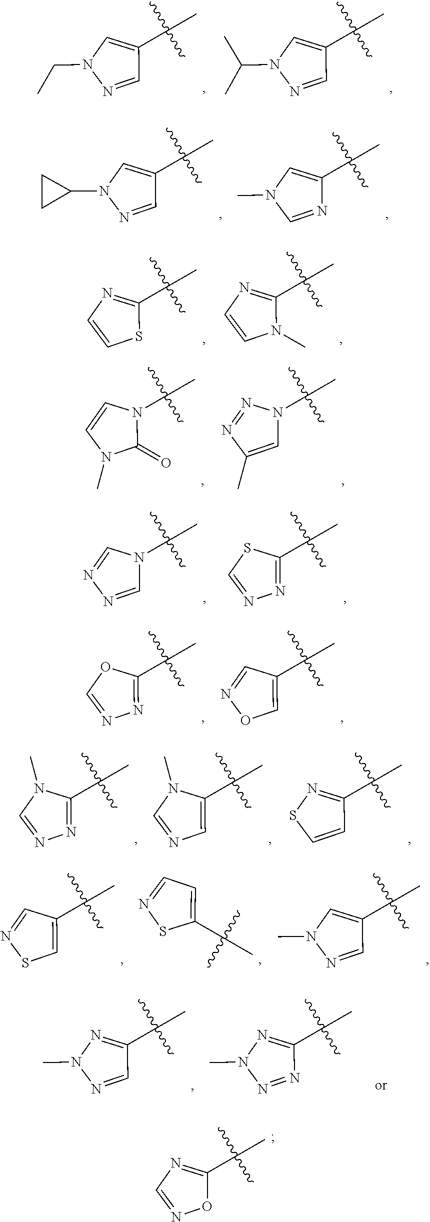

in certain embodiments, R₁₂ is selected from

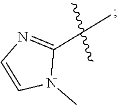 [SMILES: *c1nccn1C]

in certain embodiments, R₁₂ is selected from

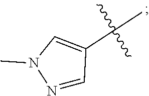 [SMILES: *c1cnn(C)c1]

in certain embodiments, R₁₂ is selected from

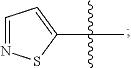 [SMILES: *c1ccns1]

and in certain embodiments, R₁₂ is selected from

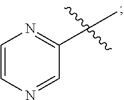 [SMILES: *c1cnccn1]

Y₁ is selected from

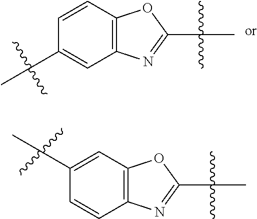 [SMILES: *c1ccc2oc(*)nc2c1 ; *c1ccc2nc(*)oc2c1]

or N optionally substituted 0-3 R^{y1};
R^{y1} is selected from F, methyl, difluoromethyl or trifluoromethyl;
Y₃ is selected from

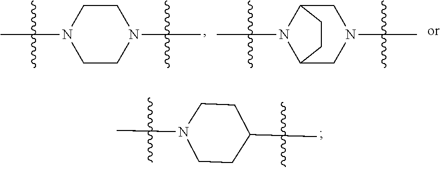 [SMILES: *N1CCN(*)CC1 ; *N1CC2CCC(C1)N2* ; *C1CCN(*)CC1]

Y₂ is selected from

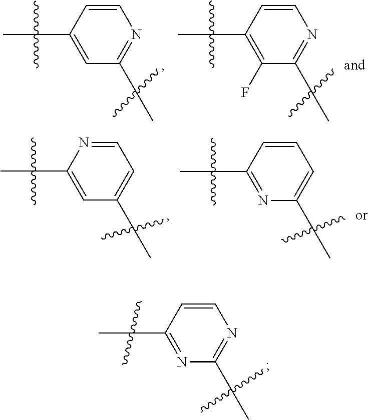 [SMILES: *c1ccnc(*)c1 ; *c1ccnc(*)c1F ; *c1ccnc(*)c1 ; *c1cccc(*)n1 ; *c1ccnc(*)n1]

and L₁ is selected from a bond,

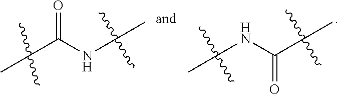 [SMILES: *NC(*)=O ; *NC(*)=O]

For the compound of formula (I-2), the isomer thereof or the pharmaceutically acceptable salt thereof of the present disclosure, R₁₂ is selected from

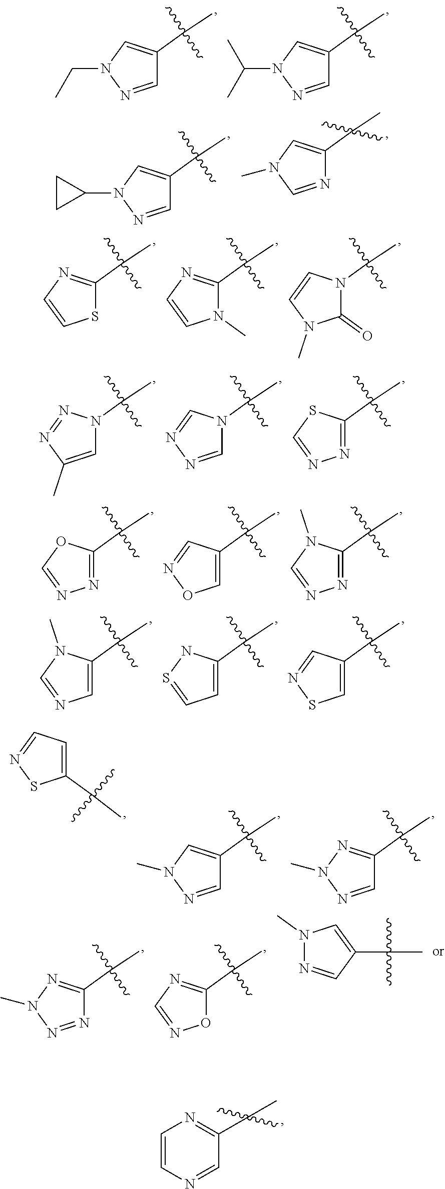

or R₁₂ is selected from

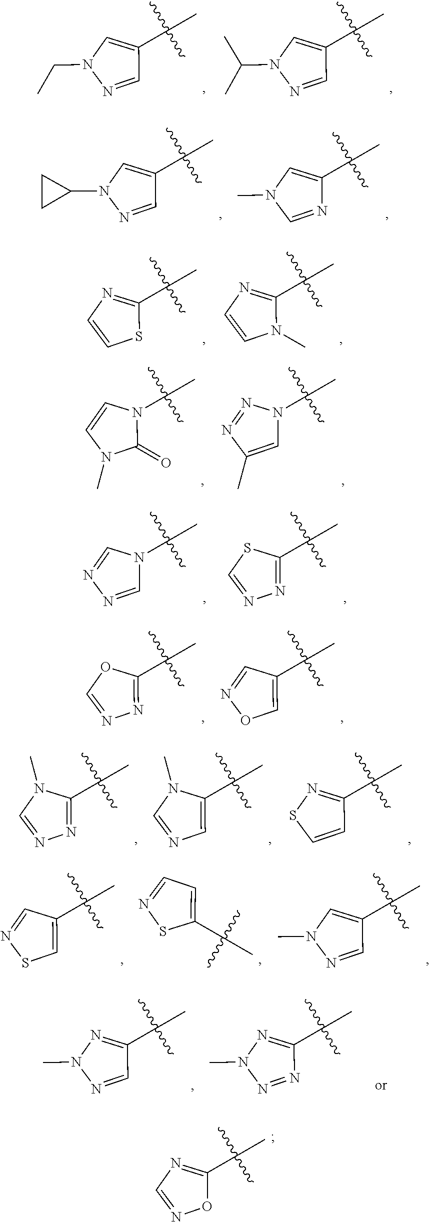

or R₁₂ is selected from

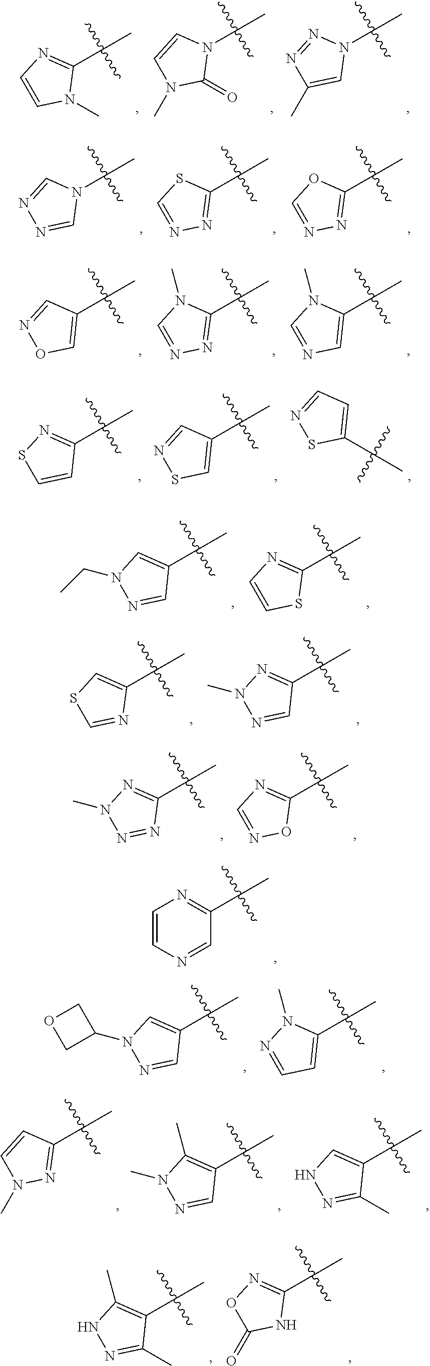

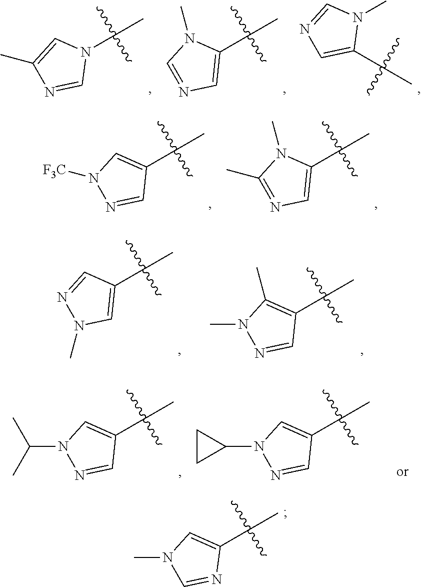

Y₁ is selected from

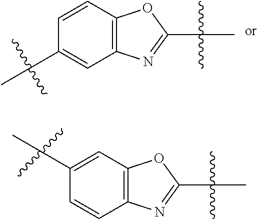 [SMILES: *c1ccc2oc(*)nc2c1 ; *c1ccc2nc(*)oc2c1]

optionally substituted 0-3 R^{y1};
R^{y1} is selected from F, methyl, difluoromethyl or trifluoromethyl;
Y₃ is selected from

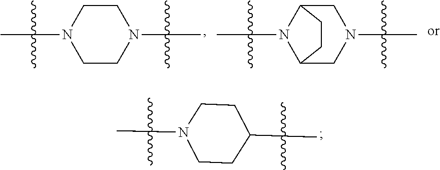 [SMILES: *N1CCN(*)CC1 ; *N1CC2CCC(C1)N2* ; *C1CCN(*)CC1]

Y₂ is selected from

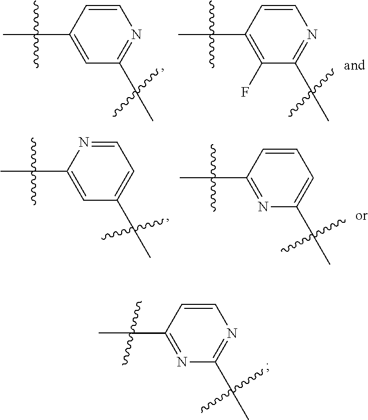 [SMILES: *c1ccnc(*)c1 ; *c1ccnc(*)c1F ; *c1ccnc(*)c1 ; *c1cccc(*)n1 ; *c1ccnc(*)n1]

and L₁ is selected from a bond,

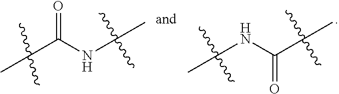 [SMILES: *NC(*)=O ; *NC(*)=O]

Embodiment ten of the present disclosure relates to a compound of formula (I-a), an isomer thereof or a pharmaceutically acceptable salt thereof,

R¹² is selected from 3-8 membered heterocyclyl, 5-10 membered heteroaryl or

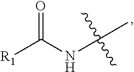

wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy, C₃₋₈ cycloalkyl or C₁₋₈ haloalkyl;
R₁ is selected from C₃₋₈ cycloalkyl, wherein the cycloalkyl is optionally further substituted with 0-3 halogen groups;
R^{y1} is selected from halogen, cyano and C₁₋₈ haloalkyl;
and the definitions of other groups are consistent with those of embodiment five of the present disclosure.

The compound of formula (I-a), the isomer thereof or the pharmaceutically acceptable salt thereof of the present disclosure has a structure of formula (I-3) or formula (I-3-1),

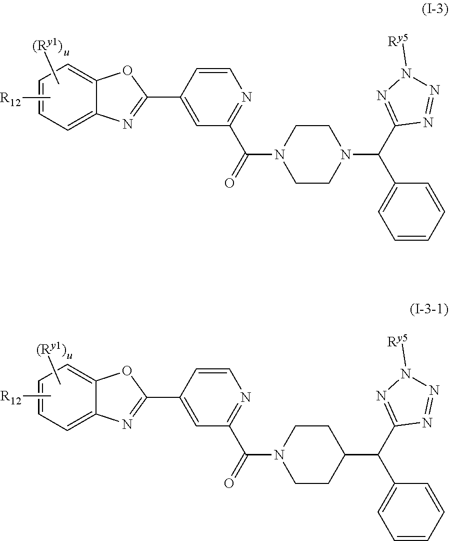

wherein,
R₁₂ is selected from 3-8 membered heterocyclyl, 5-10 membered heteroaryl and

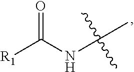

wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy, C₃₋₈ cycloalkyl or C₁₋₈ haloalkyl; in certain embodiments, R₁₂ is selected from 3-8 membered heterocyclyl, 5-10 membered heteroaryl and

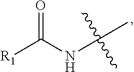

wherein the heterocyclyl and heteroaryl are optionally substituted with 0 to 3 of the following groups: halogen, ═O, acetamido, hydroxyl, C₁₋₈ alkyl and C₃₋₈ cycloalkyl; in certain embodiments, R₁₂ is selected from

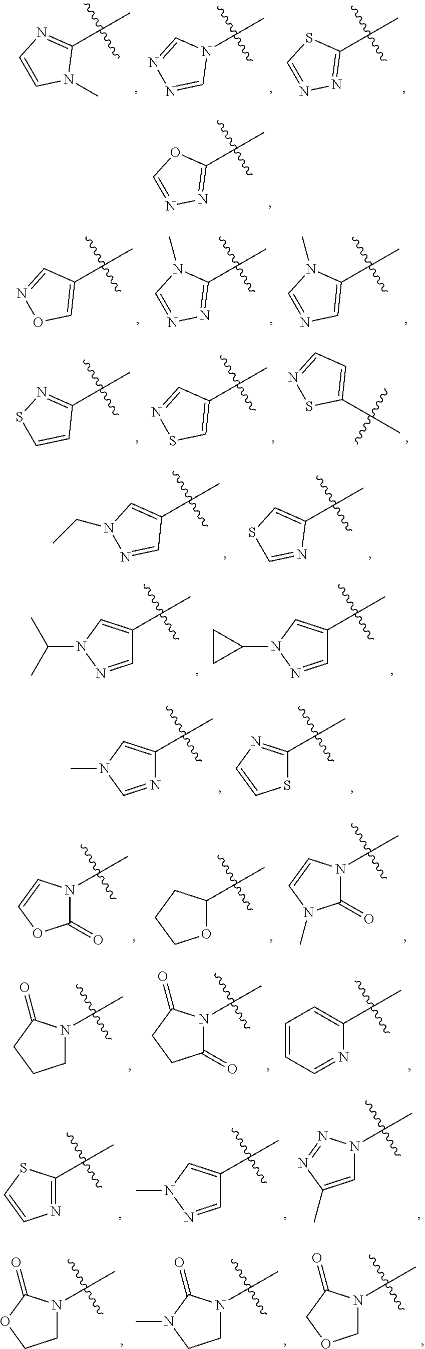

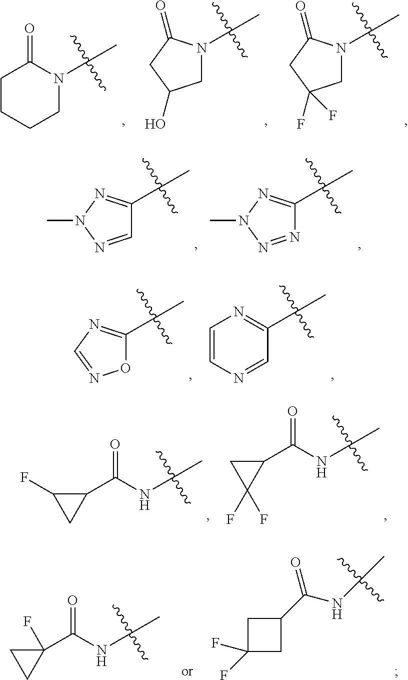

in certain embodiments, R₁₂ is selected from

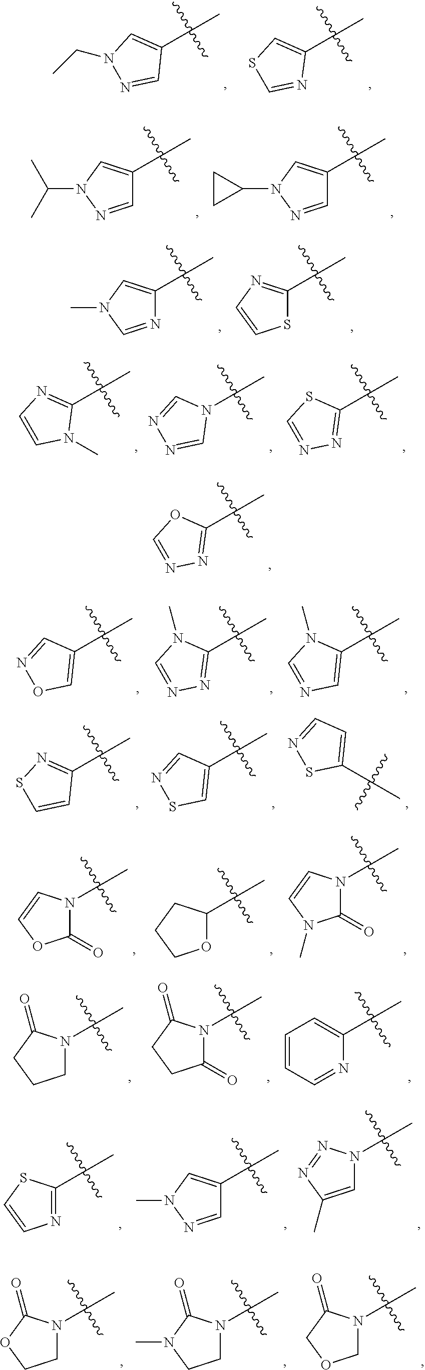

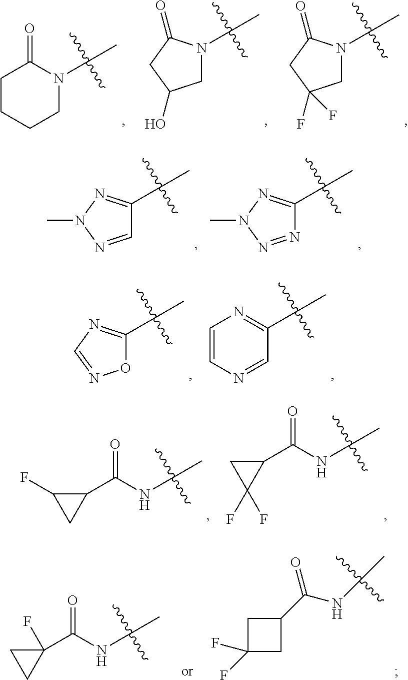

in certain embodiments, R₁₂ is selected from

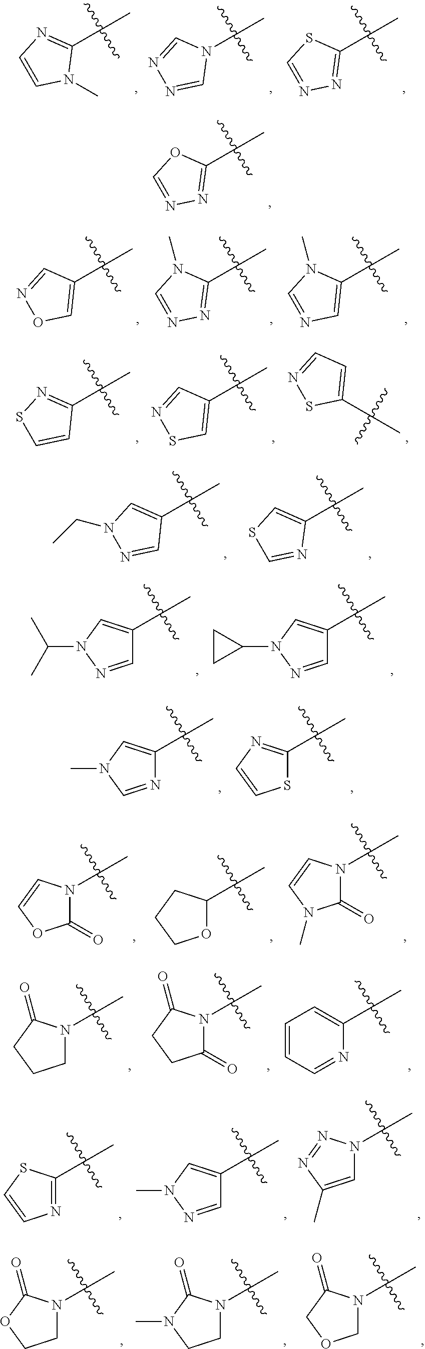

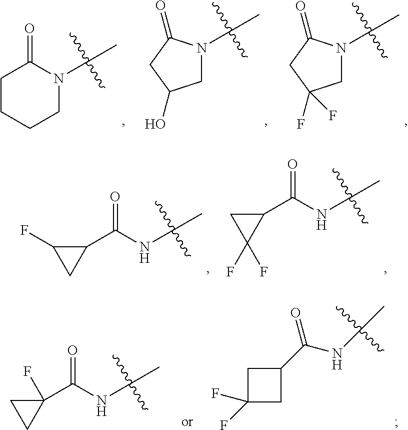

in certain embodiments, R₁₂ is selected from

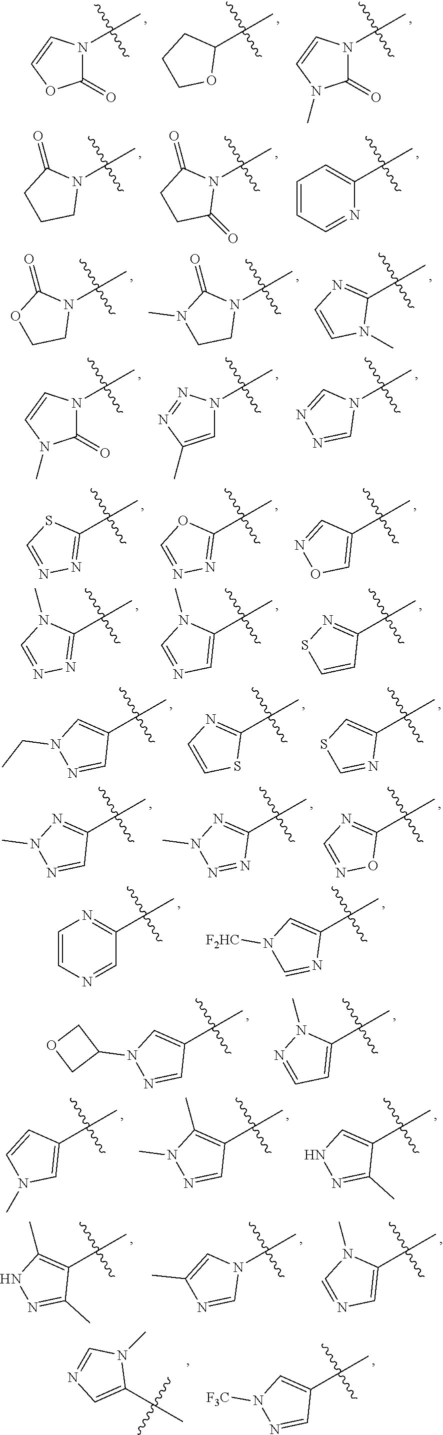

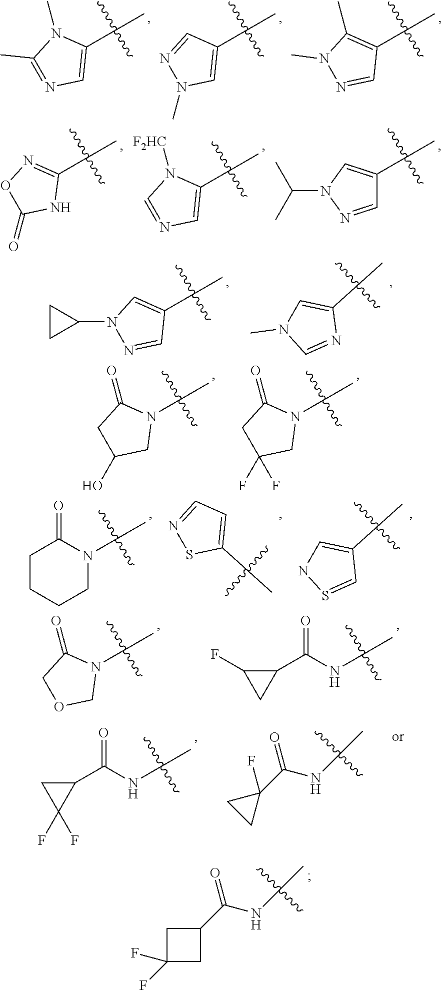

in certain embodiments, R₁₂ is selected from

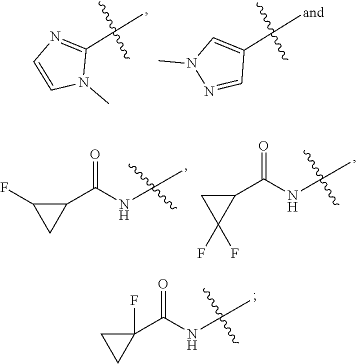

in certain embodiments, R₁₂ is selected from

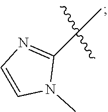 [SMILES: *c1nccn1C]

in certain embodiments, R₁₂ is selected from

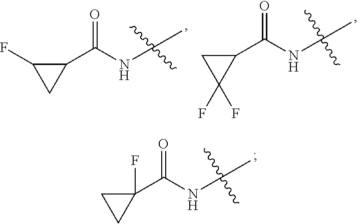 [SMILES: *NC(=O)C1CC1F ; *NC(=O)C1CC1(F)F ; *NC(=O)C1(F)CC1]

in certain embodiments, R₁₂ is selected from

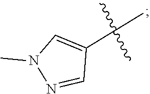 [SMILES: *c1cnn(C)c1]

R₁ is selected from C₃₋₈ cycloalkyl, wherein the cycloalkyl is optionally further substituted with 0-3 halogen groups; in certain embodiments, R₁ is selected from cyclopropyl, wherein the cyclopropyl is optionally further substituted with 0-3 halogen groups;
R^{y1} is selected from F, methyl, difluoromethyl or trifluoromethyl;
R^{y5} is selected from C₁₋₈ alkyl, C₁₋₈ haloalkyl or C₃₋₈ cycloalkyl; in certain embodiments, R^{y5} is selected from methyl, difluoromethyl and fluoroethyl; in certain embodiments, R^{y5} is selected from methyl and difluoromethyl; in certain embodiments, R^{y5} is selected from methyl; in certain embodiments, R^{y5} is selected from difluoromethyl;
u is selected from 0, 1 or 2; in certain embodiments, u is selected from 0 or 1; and in certain embodiments, u is selected from 0.

The compound of formula (I-a), the isomer thereof or the pharmaceutically acceptable salt thereof of the present disclosure has a structure of formula (I-3),

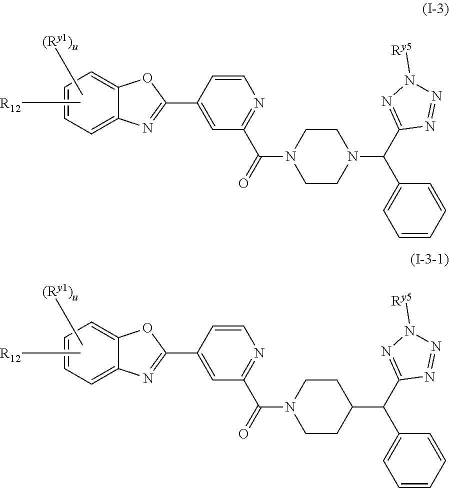

wherein,
R₁₂ is selected from 3-8 membered heterocyclyl, 5-10 membered heteroaryl and

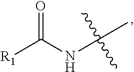

wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy or C₁₋₈ haloalkyl;
R^{y1} is selected from F, methyl, difluoromethyl or trifluoromethyl;
R^{y5} is selected from C₁₋₈ alkyl, C₁₋₈ haloalkyl or C₃₋₈ cycloalkyl; in certain embodiments, R^{y5} is selected from C₁₋₈ alkyl or C₃₋₈ cycloalkyl; and u is selected from 0, 1 or 2.

The compound of formula (I-a), the isomer thereof or the pharmaceutically acceptable salt thereof of the present disclosure has a structure of formula (I-3),

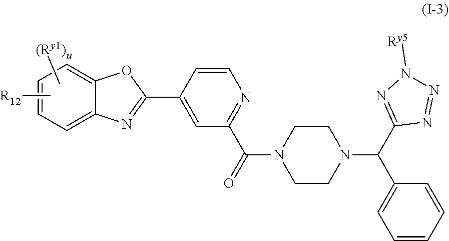

wherein,
R₁₂ is selected from 3-8 membered heterocyclyl, 5-10 membered heteroaryl and

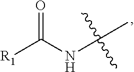

wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy or C₁₋₈ haloalkyl;
R^{y1} is selected from F, methyl, difluoromethyl or trifluoromethyl;
R^{y5} is selected from C₁₋₈ alkyl, C₁₋₈ haloalkyl or C₃₋₈ cycloalkyl; in certain embodiments, R^{y5} is selected from C₁₋₈ alkyl or C₃₋₈ cycloalkyl; and u is selected from 0, 1 or 2.

For the compound of formula (I-a), the isomer thereof or the pharmaceutically acceptable salt thereof of the present disclosure, R₁₂ is selected from

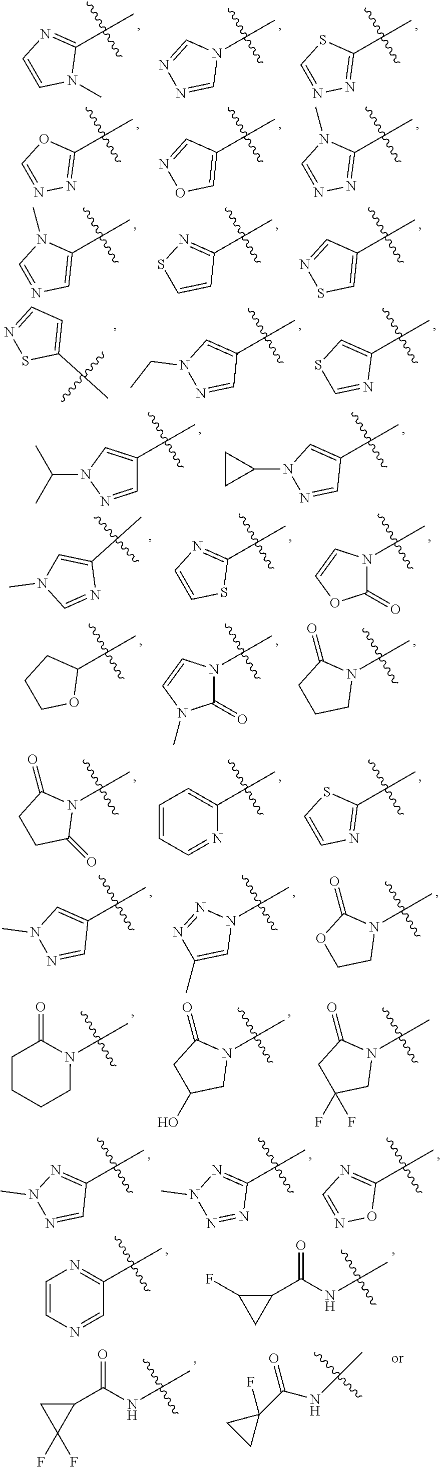

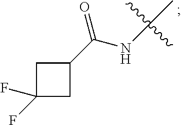 [SMILES: *NC(=O)C1CC(F)(F)C1]

or R₁₂ is selected from

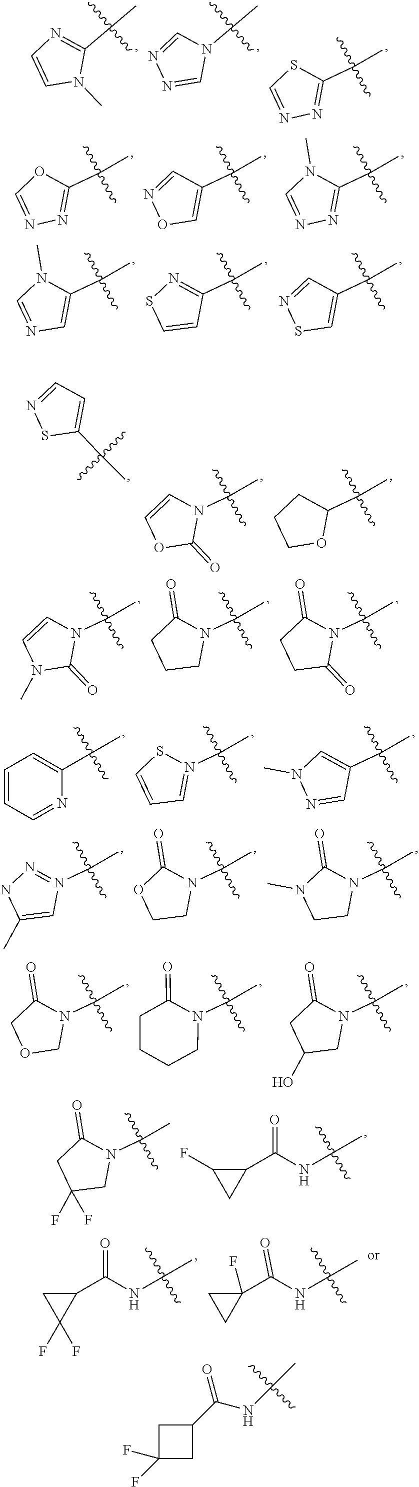

or R₁₂ is selected from

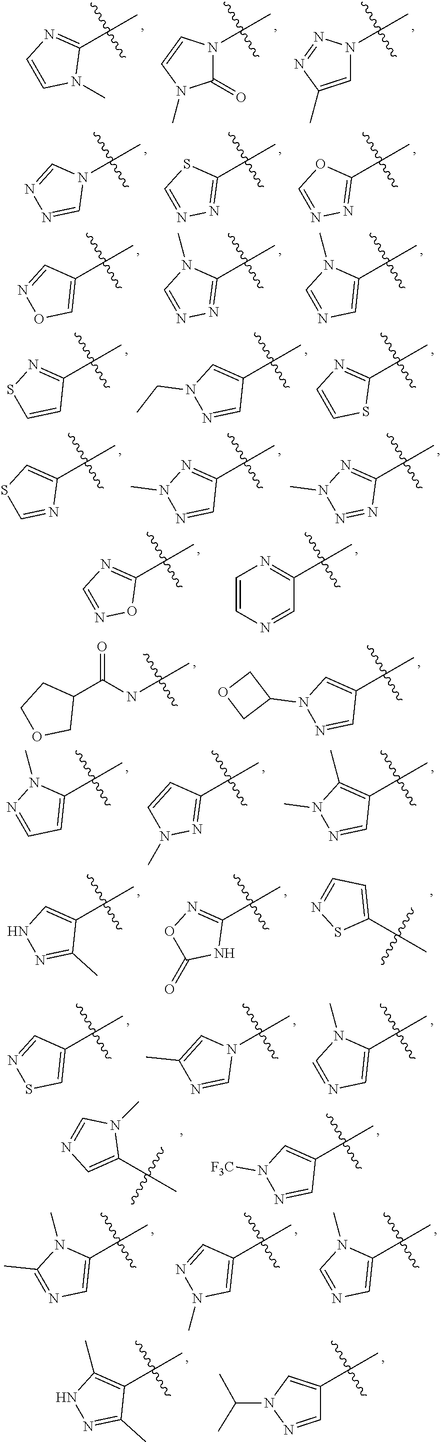

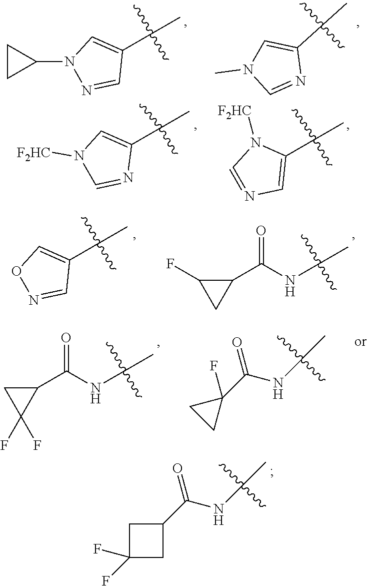

and the definitions of other groups are consistent with those of embodiment five of the present disclosure.

For the compound of formula (I-3), the isomer thereof or the pharmaceutically acceptable salt thereof of the present disclosure, R₁₂ is selected from

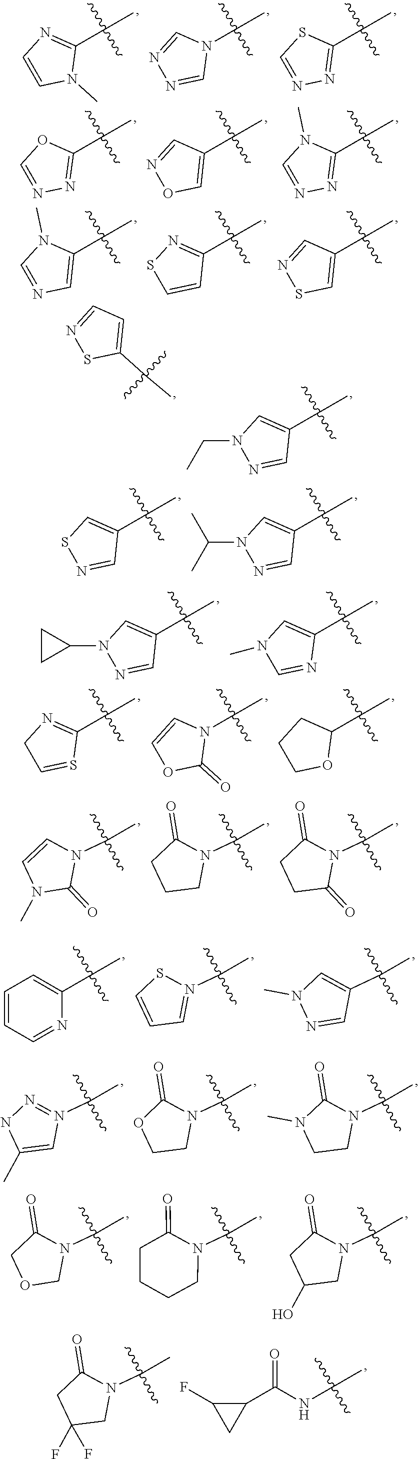

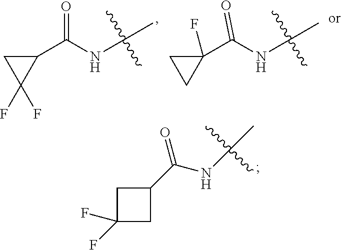 [SMILES: *NC(=O)C1CC1(F)F ; *NC(=O)C1(F)CC1 ; *NC(=O)C1CC(F)(F)C1]

in certain embodiments, R₁₂ is selected from

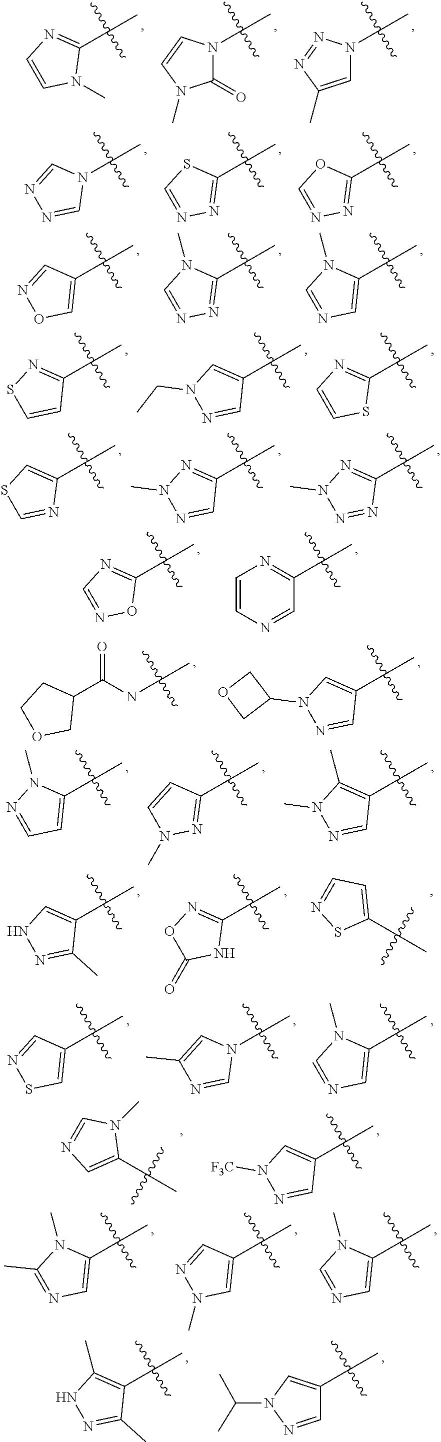

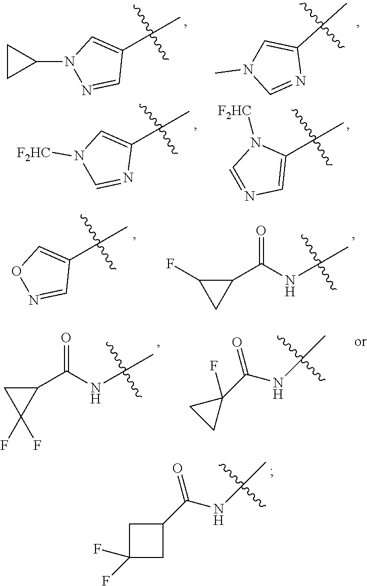

in certain embodiments, R₁₂ is selected from

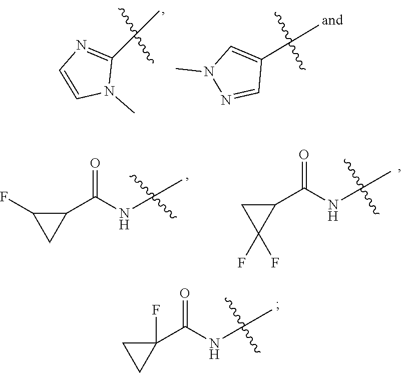

and in certain embodiments, R₁₂ is selected from

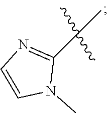 [SMILES: *c1nccn1C]

in certain embodiments, R₁₂ is selected from

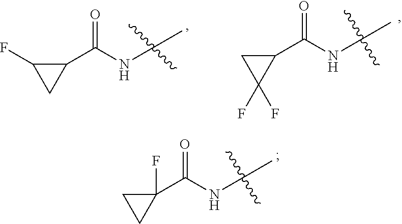 [SMILES: *NC(=O)C1CC1F ; *NC(=O)C1CC1(F)F ; *NC(=O)C1(F)CC1]

in certain embodiments, R₁₂ is selected from

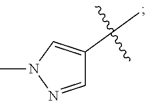 [SMILES: *c1cnn(C)c1]

in certain embodiments, R₁₂ is selected from

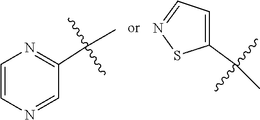 [SMILES: *c1cnccn1 ; *c1ccns1]

R^{y1} is selected from F, methyl, difluoromethyl or trifluoromethyl;
R^{y5} is selected from C₁₋₈ alkyl, C₁₋₈ haloalkyl or C₃₋₈ cycloalkyl; in certain embodiments, R^{y5} is selected from methyl, difluoromethyl and fluoroethyl; in certain embodiments, R^{y5} is selected from methyl and difluoromethyl; in certain embodiments,
R^{y5} is selected from methyl; in certain embodiments, R^{y5} is selected from difluoromethyl;
u is selected from 0, 1 or 2; in certain embodiments, u is selected from 0 or 1; and in certain embodiments, u is selected from 0.

For the compound of formula (I-3), the isomer thereof or the pharmaceutically acceptable salt thereof of the present disclosure, R¹² is selected from 3-8 membered heterocyclyl, 5-10 membered heteroaryl or

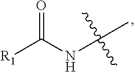

wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy or C₁₋₈ haloalkyl;
R₁ is selected from C₃₋₈ cycloalkyl, wherein the cycloalkyl is optionally further substituted with 0-3 halogen groups;
R^{y1} is selected from halogen, cyano and C₁₋₈ haloalkyl;
R^{y5} is selected from C₁₋₈ alkyl, C₁₋₈ haloalkyl or C₃₋₈ cycloalkyl; in certain embodiments, R^{y5} is selected from methyl, difluoromethyl and fluoroethyl; in certain embodiments, R^{y5} is selected from methyl and difluoromethyl; in certain embodiments, R^{y5} is selected from methyl; in certain embodiments, R^{y5} is selected from difluoromethyl;
u is selected from 0, 1 or 2; in certain embodiments, u is selected from 0 or 1; and in certain embodiments, u is selected from 0.

Embodiment eleven of the present disclosure relates to a compound of formula (I-a), an isomer thereof or a pharmaceutically acceptable salt thereof, having a structure of formula (I-4), wherein

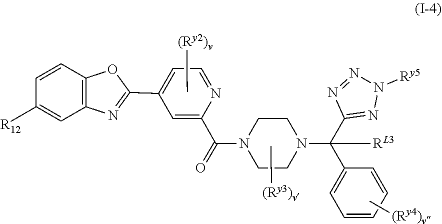

R₁₂ is selected from cyano, C₁₋₈ alkoxy and

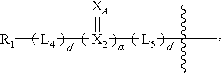

wherein the alkoxy is optionally substituted with 0-5 halogen substituents; in certain embodiments, R₁₂ is selected from C₁₋₈ alkoxy and

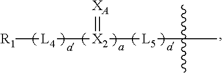

wherein the alkoxy is optionally substituted with 0-5 halogen substituents; in certain embodiments, R₁₂ is selected from

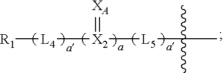

X₂ is selected from C;
L₄ and L₅ are each independently selected from: NR₂, CR₃R₄ or O; in certain embodiments, L₄ and L₅ are each independently selected from: NR₂ or O;
X_{A} is selected from O; a is 0 or 1; each a′ is independently selected from 0 or 1;
R₁ is selected from: H, halogen, sulfhydryl, cyano, nitro, C₁₋₈ alkyl, C₃₋₈ cycloalkyl, C₂₋₈ alkenyl, C₂₋₈ alkynyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: halogen, C₁₋₈ alkyl, cyano, ═O, C₁₋₈ alkoxy or C₁₋₈ haloalkyl; in certain embodiments, R₁ is selected from: H, C₁₋₈ alkyl, C₁₋₈ alkoxy, C₃₋₈ cycloalkyl, 3-8 membered heterocyclyl or 5-10 membered heteroaryl, wherein the alkyl, alkoxy, cycloalkyl, heterocyclyl or heteroaryl is optionally further substituted with 0-5 groups selected from: halogen, C₁₋₈ alkyl, cyano, ═O or C₁₋₈ haloalkyl;
R₂, R₃ and R₄ are each independently selected from: H or C₁₋₈ alkyl; in certain embodiments, R₂, R₃ and R₄ are each independently selected from: H;
R^{y2} is selected from halogen, cyano, C₁₋₈ alkyl, C₁₋₈ haloalkyl or C₁₋₈ alkoxy; in certain embodiments, R^{y2} is selected from halogen, cyano and methyl; in certain embodiments,
R^{y2} is selected from cyano;
R^{y3} is selected from halogen, cyano, ═O, C₁₋₈ alkyl, C₁₋₈ haloalkyl and C₁₋₈ alkoxy, or together with the carbon atoms on the piperazine ring to which they are attached forms cyclopropyl; in certain embodiments, R^{y3} is selected from halogen, ═O and methyl, or together with the carbon atoms on the piperazine ring to which they are attached forms cyclopropyl; in certain embodiments, R^{y3} is selected from halogen, ═O and methyl;
R^{y4} is selected from halogen, cyano, C₁₋₈ alkyl, C₁₋₈ haloalkyl, C₁₋₈ alkoxy or C₃₋₈ cycloalkyl; in certain embodiments, R^{y4} is selected from halogen, cyano, methoxy and trifluoromethyl;
R^{y5} is selected from C₁₋₈ alkyl or C₃₋₈ cycloalkyl; in certain embodiments, R^{y5} is selected from methyl;
R^{L3} is selected from H;
v, v′ and v″ are each independently selected from 0, 1 or 2; and in certain embodiments, v, v′ and v″ are each independently selected from 0 and 1;
provided that:
when R^{y5} is selected from methyl and R₁₂ is selected from acetamido, halogen, methyl, methoxy, trifluoromethyl, trifluoromethyloxy, hydroxyl, methanesulfonylamido, methylamino or N,N-dimethylamino, R^{L3} is not selected from H, or v, v′ and v″ are not simultaneously 0;
and the definitions of other groups are consistent with those of embodiment five of the present disclosure.

The compound of formula (I-a), the isomer thereof or the pharmaceutically acceptable salt thereof of embodiment eleven of the present disclosure has a structure of formula (I-4), wherein

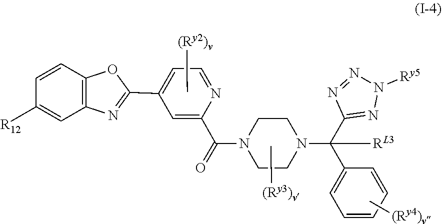

R₁₂ is selected from cyano, C₁₋₈ alkoxy and

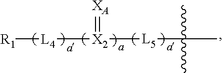

wherein the alkoxy is optionally substituted with 0-5 halogen substituents;
X₂ is selected from C;
L₄ and L₅ are each independently selected from: NR₂, CR₃R₄ or 0;
X_{A} is selected from O; a is 0 or 1;
each a′ is independently selected from 0 or 1;
R₁ is selected from: H, halogen, sulfhydryl, cyano, nitro, C₁₋₈ alkyl, C₃₋₈ cycloalkyl, C₂₋₈ alkenyl, C₂₋₈ alkynyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: halogen, C₁₋₈ alkyl, cyano, ═O, C₁₋₈ alkoxy or C₁₋₈ haloalkyl;
R₂, R₃ and R₄ are each independently selected from: H or C₁₋₈ alkyl;
R^{y2} is selected from halogen, cyano, C₁₋₈ alkyl, C₁₋₈ haloalkyl or C₁₋₈ alkoxy;
R^{y3} is selected from halogen, cyano, ═O, C₁₋₈ alkyl, C₁₋₈ haloalkyl and C₁₋₈ alkoxy, or together with the carbon atoms on the piperazine ring to which they are attached forms cyclopropyl;
R^{y4} is selected from halogen, cyano, C₁₋₈ alkyl, C₁₋₈ haloalkyl, C₁₋₈ alkoxy or C₃₋₈ cycloalkyl;
R^{y5} is selected from C₁₋₈ alkyl or C₃₋₈ cycloalkyl;
R^{L3} is selected from H;
v, v′ and v″ are each independently selected from 0, 1 or 2; provided that:
when R^{y5} is selected from methyl and R₁₂ is selected from acetamido, halogen, methyl, methoxy, trifluoromethyl, trifluoromethyloxy, hydroxyl, methanesulfonylamido, methylamino or N,N-dimethylamino, R^{L3} is not selected from H, or v, v′ and v″ are not simultaneously 0;
and the definitions of other groups are consistent with those of embodiment five of the present disclosure.

For the compound of (I-4), the isomer thereof or the pharmaceutically acceptable salt thereof of the present disclosure,

R₁₂ is selected from cyano,

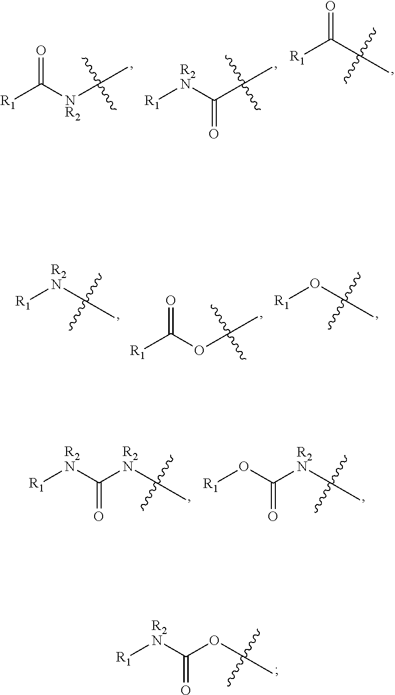

R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: hydroxyl, halogen, cyano, ═O, C₁₋₈ alkyl, C₁₋₈ alkoxy or C₁₋₈ haloalkyl; each R₂ is independently selected from: H or C₁₋₈ alkyl;
R^{y2} is selected from halogen, cyano, C₁₋₈ alkyl, C₁₋₈ haloalkyl or C₁₋₈ alkoxy;
R^{y3} is selected from halogen, cyano, ═O, C₁₋₈ alkyl, C₁₋₈ haloalkyl and C₁₋₈ alkoxy, or together with the carbon atoms on the piperazine ring to which they are attached forms cyclopropyl;
R^{y4} is selected from halogen, cyano, C₁₋₈ alkyl, C₁₋₈ haloalkyl, C₁₋₈ alkoxy or C₃₋₈ cycloalkyl;
R^{y5} is selected from C₁₋₈ alkyl or C₃₋₈ cycloalkyl;
R^{L3} is selected from H;
v, v′ and v″ are each independently selected from 0, 1 or 2;
provided that:
when R^{y5} is selected from methyl and R₁₂ is selected from acetamido, halogen, methyl, methoxy, trifluoromethyl, trifluoromethyloxy, hydroxyl, methanesulfonylamido, methylamino or N,N-dimethylamino, R^{L3} is not selected from H, or v, v′ and v″ are not simultaneously 0;
and the definitions of other groups are consistent with those of embodiment five of the present disclosure.

For the compound of (I-4), the isomer thereof or the pharmaceutically acceptable salt thereof of the present disclosure,

R₁₂ is selected from acetamido, C₁₋₈ haloalkoxy, —O-3-8 membered heterocyclyl, —C₁₋₈ alkyl-O—C(O)NH—, —C₁₋₈ haloalkyl-O—C(O)NH—, C₃₋₈ cycloalkyl-NH—,

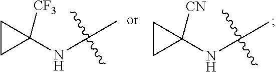 [SMILES: *NC1(C(F)(F)F)CC1 ; *NC1(C#N)CC1]

in certain embodiments, R₁₂ is selected from acetamido, difluoromethyloxy, oxacyclopentyloxy, methoxycarboxamido;
R^{y5} is selected from methyl;
R^{L3} is selected from H;
v′ is each independently selected from 0, 1 or 2; in certain embodiments, v′ is each independently selected from 0 and 1;
v and v″ are each independently selected from 0;
R^{y2} is selected from halogen, cyano, C₁₋₈ alkyl, C₁₋₈ haloalkyl or C₁₋₈ alkoxy;
R^{y3} is selected from halogen, cyano, ═O, C₁₋₈ alkyl, C₁₋₈ haloalkyl and C₁₋₈ alkoxy, or together with the carbon atoms on the piperazine ring to which they are attached forms cyclopropyl;
and R^{y4} is selected from halogen, cyano, C₁₋₈ alkyl, C₁₋₈ haloalkyl, C₁₋₈ alkoxy or C₃₋₈ cycloalkyl, and in certain embodiments, R^{y4} is selected from halogen;
provided that:
when R^{y5} is selected from methyl and R₁₂ is selected from acetamido, halogen, methyl, methoxy, trifluoromethyl, trifluoromethyloxy, hydroxyl, methanesulfonylamido, methylamino or N,N-dimethylamino, R^{L3} is not selected from H, or v, v′ and v″ are not simultaneously 0;
and the definitions of other groups are consistent with those of embodiment five of the present disclosure.

For the compound of (I-4), the isomer thereof or the pharmaceutically acceptable salt thereof of the present disclosure,

R₁₂ is selected from methoxycarboxamide, difluoromethoxy or —O-oxacyclopentyl;
R^{y2} is selected from halogen, cyano, C₁₋₈ alkyl, C₁₋₈ haloalkyl or C₁₋₈ alkoxy;
R^{y3} is selected from halogen, cyano, ═O, C₁₋₈ alkyl, C₁₋₈ haloalkyl and C₁₋₈ alkoxy, or together with the carbon atoms on the piperazine ring to which they are attached forms cyclopropyl;
R^{y4} is selected from halogen, cyano, C₁₋₈ alkyl, C₁₋₈ haloalkyl, C₁₋₈ alkoxy or C₃₋₈ cycloalkyl;
R^{y5} is selected from C₁₋₈ alkyl or C₃₋₈ cycloalkyl;
R^{L3} is selected from H;
v, v′ and v″ are each independently selected from 0, 1 or 2;
and the definitions of other groups are consistent with those of embodiment five of the present disclosure.

For the compound of (I-4), the isomer thereof or the pharmaceutically acceptable salt thereof of the present disclosure,

R₁₂ is selected from methoxycarboxamide, difluoromethoxy or —O-oxacyclopentyl;
R^{y5} is selected from methyl;
R^{L3} is selected from H;
v′ is each independently selected from 0, 1 or 2; in certain embodiments, v′ is each independently selected from 0 and 1;
v and v″ are each independently selected from 0;
R^{y2} is selected from halogen, cyano, C₁₋₈ alkyl, C₁₋₈ haloalkyl or C₁₋₈ alkoxy;
R^{y3} is selected from halogen, cyano, ═O, C₁₋₈ alkyl, C₁₋₈ haloalkyl and C₁₋₈ alkoxy, or together with the carbon atoms on the piperazine ring to which they are attached forms cyclopropyl;
R^{y4} is selected from halogen, cyano, C₁₋₈ alkyl, C₁₋₈ haloalkyl, C₁₋₈ alkoxy or C₃₋₈ cycloalkyl, and in certain embodiments, R^{y4} is selected from halogen;
and the definitions of other groups are consistent with those of embodiment five of the present disclosure.

Embodiment twelve of the present disclosure relates to a compound of formula (I-a), an isomer thereof or a pharmaceutically acceptable salt thereof, having a structure of formula (I-5), formula (I-6), formula (I-7) or formula (I-8),

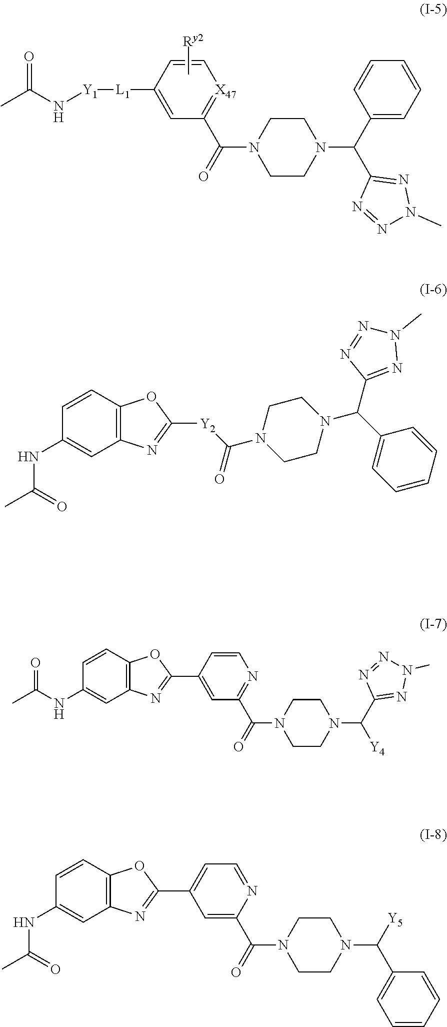

wherein,
Y₁ is selected from 5-15 membered heteroaryl, C₅₋₁₅ aryl or 3-12 membered heterocyclyl, wherein the heteroaryl, aryl or heterocyclyl is optionally substituted with 0-3 R^{y1}; in certain embodiments, Y₁ is selected from phenyl,

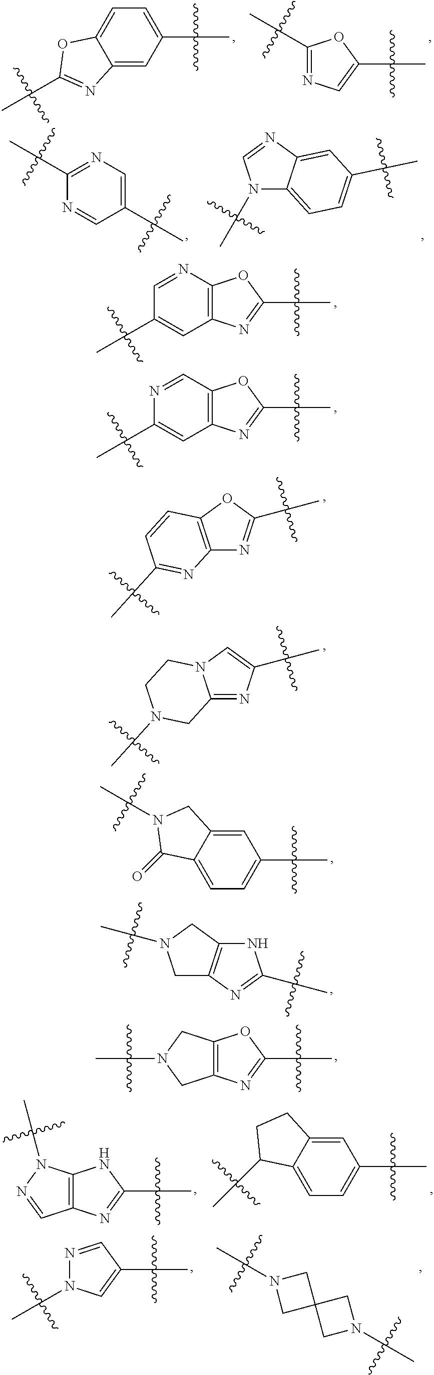

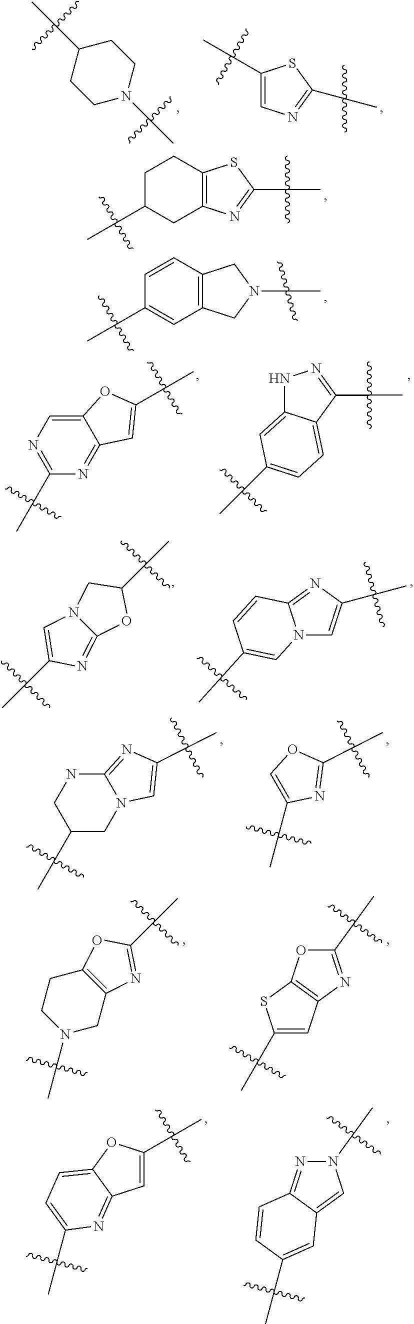

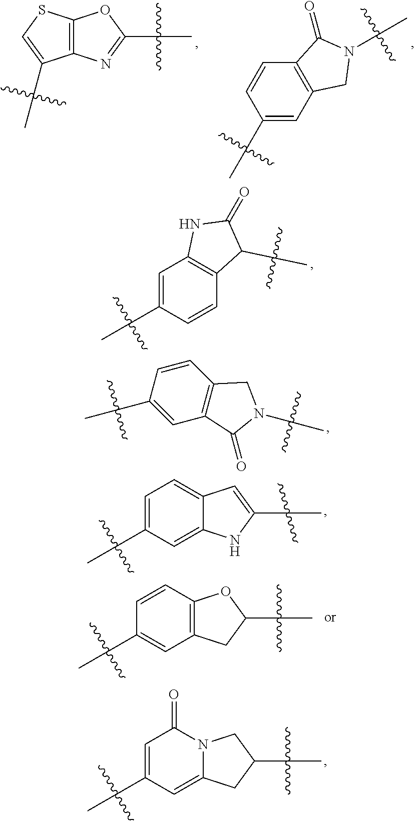

wherein the above-mentioned groups are optionally substituted with 0-3 R^{y1}; in certain embodiments, Y₁ is selected from

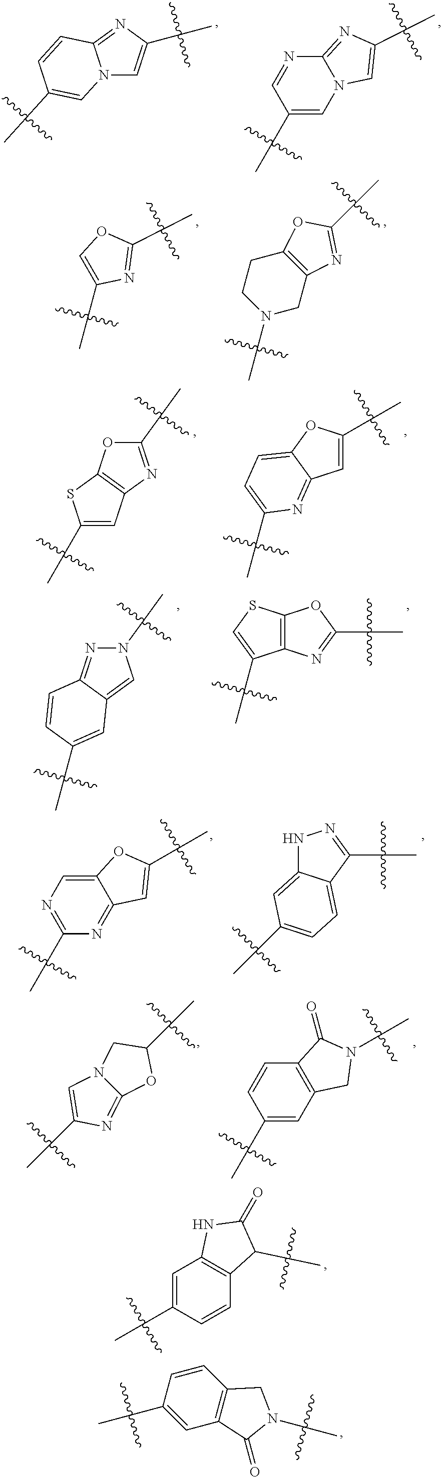

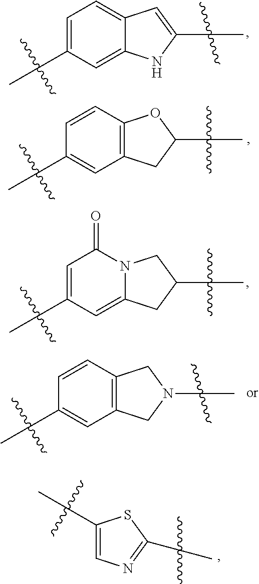

wherein the above-mentioned groups are optionally substituted with 0-3 R^{y1};
R^{y1} is selected from halogen, ═O, hydroxyl, cyano, nitro, C₁₋₈ alkyl, C₁₋₈ alkoxy or C₁₋₈ haloalkyl; in certain embodiments, R^{y1} is selected from halogen, ═O, hydroxyl and C₁₋₈ alkyl;
L₁ is selected from a bond;
R^{y2} is selected from H, halogen and cyano; or
R^{y1} and R^{y2} together with L₁ form 4-8 membered heterocyclyl, C₄-8 carbocyclyl or 5-8 membered heteroaryl; in certain embodiments, R^{y1} and R^{y2} together with L₁ form the following structure:

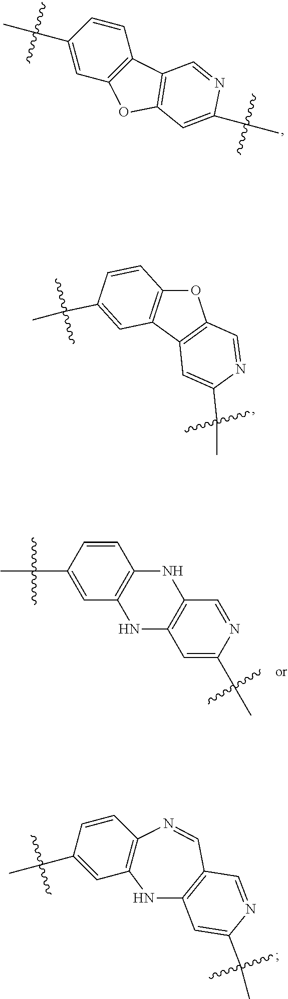 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

X₄₇ is selected from CH or N; in certain embodiments, X₄₇ is selected from N;
Y₂ is selected from 5-15 membered heteroaryl, C₅₋₁₅ aryl or 3-12 membered heterocyclyl, wherein the heteroaryl, aryl or heterocyclyl is optionally substituted with 0-3 of the following groups: halogen, ═O, hydroxyl, cyano, nitro, C₁₋₈ alkyl, C₁₋₈ alkoxy or C₁₋₈ haloalkyl; in certain embodiments, Y₂ is selected from phenyl,

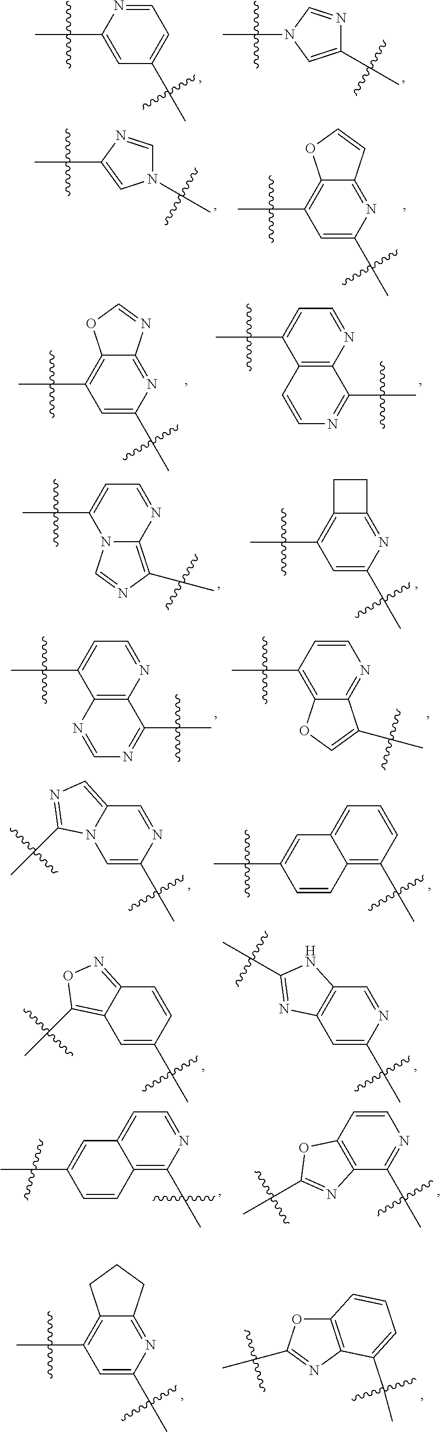

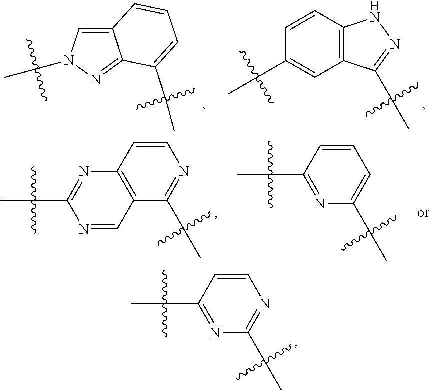

wherein the above-mentioned groups are optionally substituted with 0-3 groups selected from: C₁₋₈ haloalkyl, C₁₋₈ alkyl and cyano; in certain embodiments, Y₂ is selected from

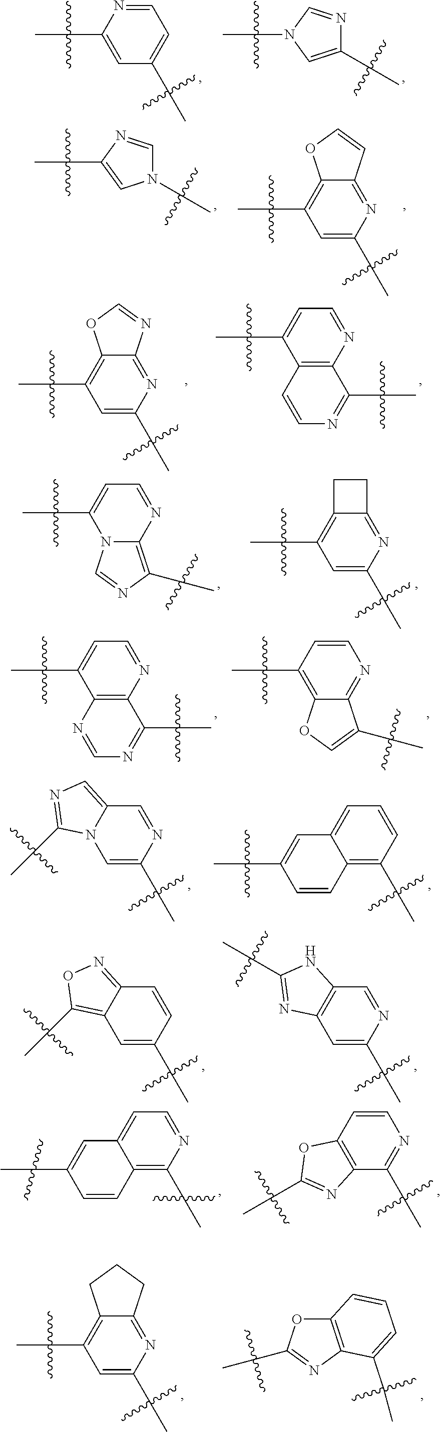

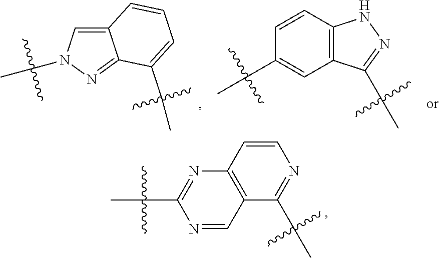 [SMILES: *c1cccc2cn(*)nc12 ; *c1ccc2[nH]nc(*)c2c1 ; *c1ncc2c(*)nccc2n1]

wherein the above-mentioned groups are optionally substituted with 0-3 groups selected from: C₁₋₈ haloalkyl, C₁₋₈ alkyl and cyano;
Y₄ is selected from 5-15 membered heteroaryl, C₅₋₁₅ aryl, 3-12 membered heterocyclyl or C₂₋₈ alkynyl, wherein the heteroaryl, aryl, heterocyclyl or alkynyl is optionally substituted with 0-3 of the following groups: halogen, ═O, hydroxyl, cyano, nitro, C₁₋₈ alkyl, C₁₋₈ alkoxy, C₃₋₈ cycloalkyl or C₁₋₈ haloalkyl; in certain embodiments, Y₄ is selected from cyclopropylethynyl, cyclopentyl, methoxyphenyl,

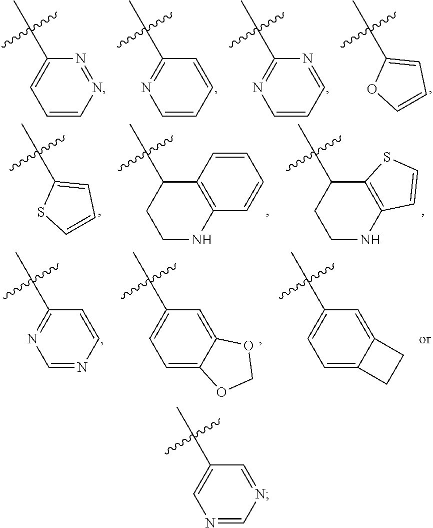

Y₅ is selected from 5-15 membered heteroaryl, 3-12 membered heterocyclyl and

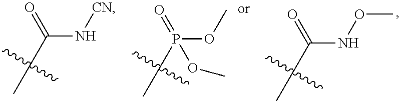

wherein the heteroaryl and heterocyclyl are optionally substituted with 0-3 of the following groups: halogen, ═O, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl; and in certain embodiments, Y₅ is selected from

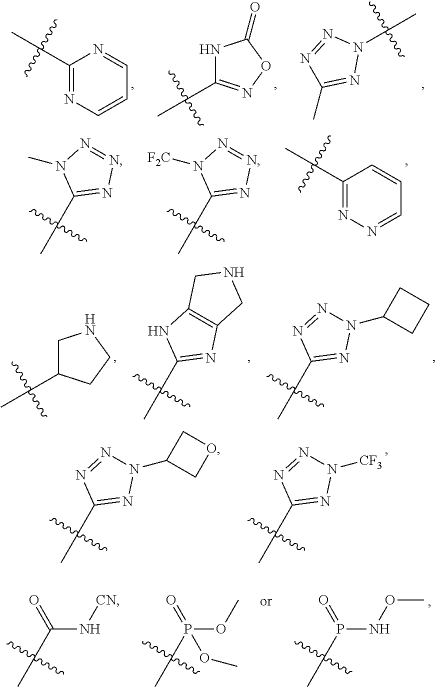

wherein the above-mentioned groups are optionally substituted with 0 to 3 of the following groups: C₁₋₈ haloalkyl and C₁₋₈ alkyl;
provided that: Y₁ is not selected from

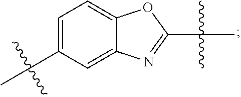 [SMILES: *c1ccc2oc(*)nc2c1]

Y₂ is not selected from

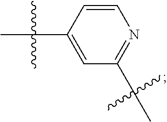 [SMILES: *c1ccnc(*)c1]

Y₄ is not selected from phenyl; and Y₅ is not selected from

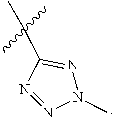 [SMILES: *c1nnn(C)n1]

The compound of formula (I-a), the isomer thereof or the pharmaceutically acceptable salt thereof of the present disclosure has a structure of formula (I-5), formula (I-6), formula (I-7) or formula (I-8),

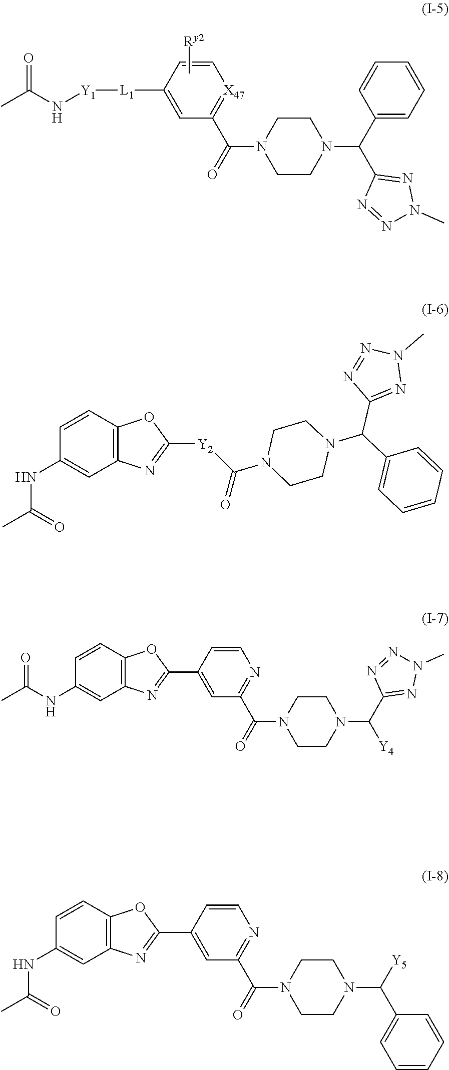

wherein,
Y₁ is selected from 5-15 membered heteroaryl, C₅₋₁₅ aryl or 3-12 membered heterocyclyl, wherein the heteroaryl, aryl or heterocyclyl is optionally substituted with 0-3 R^{y1};
R^{y1} is selected from halogen, ═O, hydroxyl, cyano, nitro, C₁₋₈ alkyl, C₁₋₈ alkoxy or C₁₋₈ haloalkyl;
L₁ is selected from a bond;
R^{y2} is selected from H, halogen and cyano; or
R^{y1} and R^{y2} together with L₁ form 4-8 membered heterocyclyl, C₄-8 carbocyclyl or 5-8 membered heteroaryl;
X₄₇ is selected from CH or N;
Y₂ is selected from 5-15 membered heteroaryl, C₅₋₁₅ aryl or 3-12 membered heterocyclyl, wherein the heteroaryl, aryl or heterocyclyl is optionally substituted with 0-3 of the following groups: halogen, ═O, hydroxyl, cyano, nitro, C₁₋₈ alkyl, C₁₋₈ alkoxy or C₁₋₈ haloalkyl;
Y₄ is selected from 5-15 membered heteroaryl, C₅₋₁₅ aryl, 3-12 membered heterocyclyl or C₂₋₈ alkynyl, wherein the heteroaryl, aryl, heterocyclyl or alkynyl is optionally substituted with 0-3 of the following groups: halogen, ═O, hydroxyl, cyano, nitro, C₁₋₈ alkyl, C₁₋₈ alkoxy, C₃₋₈ cycloalkyl or C₁₋₈ haloalkyl;
and Y₅ is selected from 5-15 membered heteroaryl, 3-12 membered heterocyclyl and

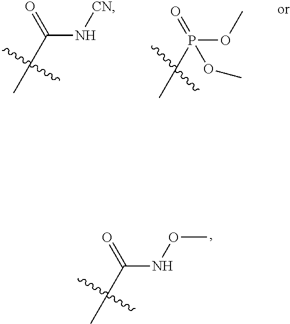

wherein the heteroaryl and heterocyclyl are optionally substituted with 0-3 of the following groups: halogen, ═O, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl;
provided that: Y₁ is not selected from

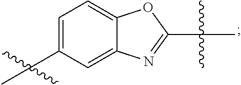 [SMILES: *c1ccc2oc(*)nc2c1]

Y₂ is not selected from

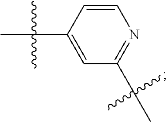 [SMILES: *c1ccnc(*)c1]

Y₄ is not selected from phenyl; and Y₅ is not selected from

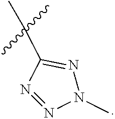 [SMILES: *c1nnn(C)n1]

For the compound of formula (I-5), formula (I-6), formula (I-7) or formula (I-8), the isomer thereof or the pharmaceutically acceptable salt thereof of the present disclosure, Y₁ is selected from phenyl,

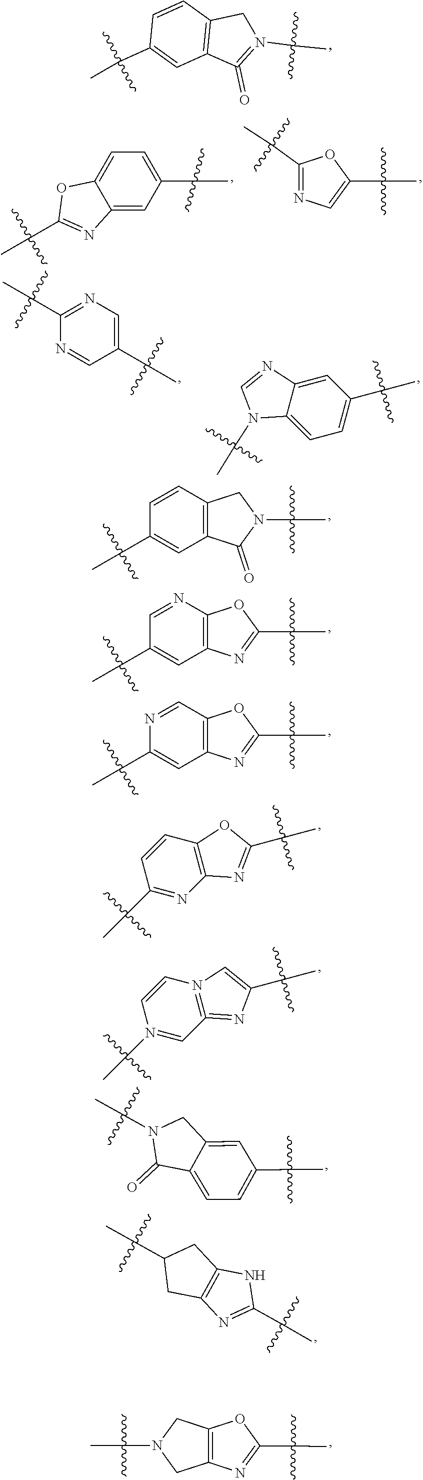

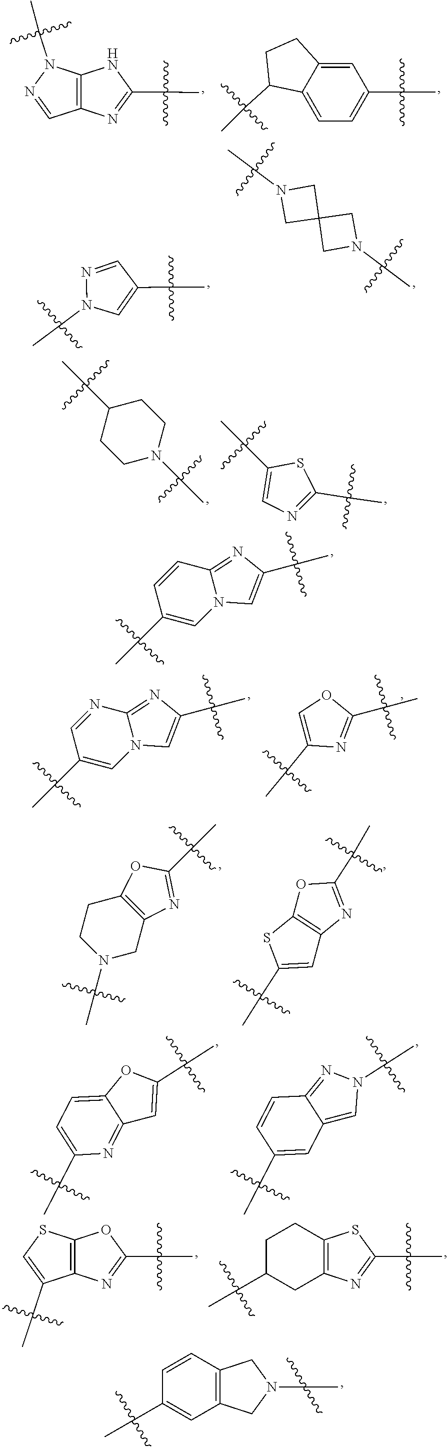

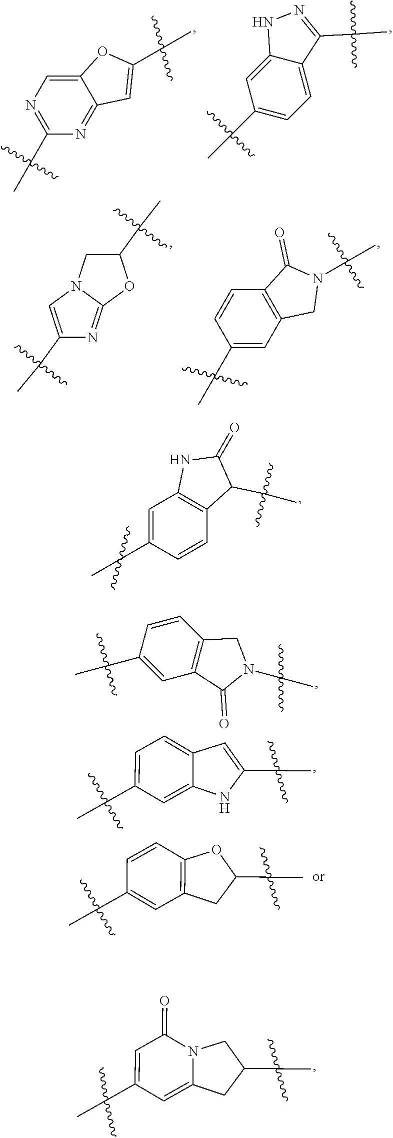

and optionally substituted with 0-3 R^{y1};
in certain embodiments, Y₁ is selected from

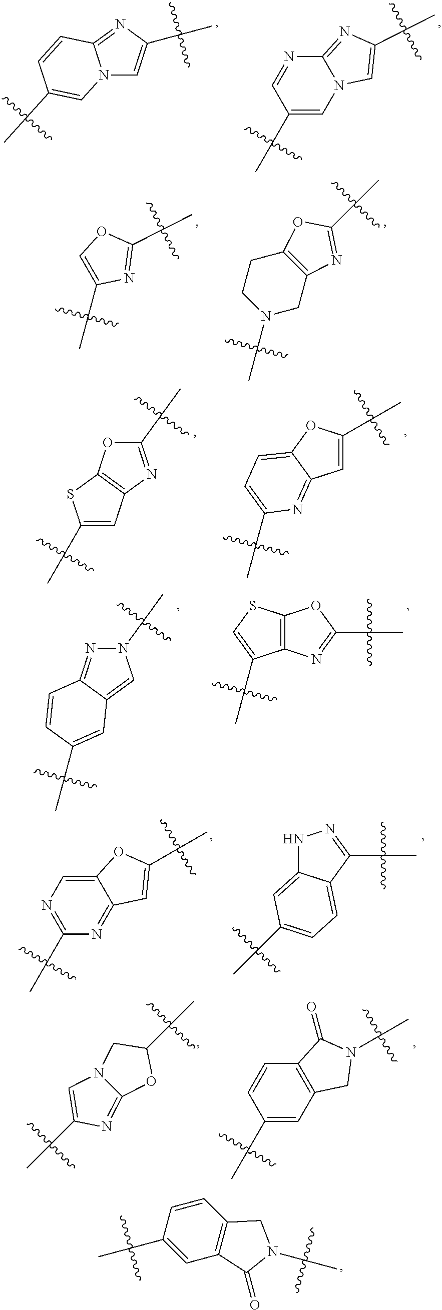

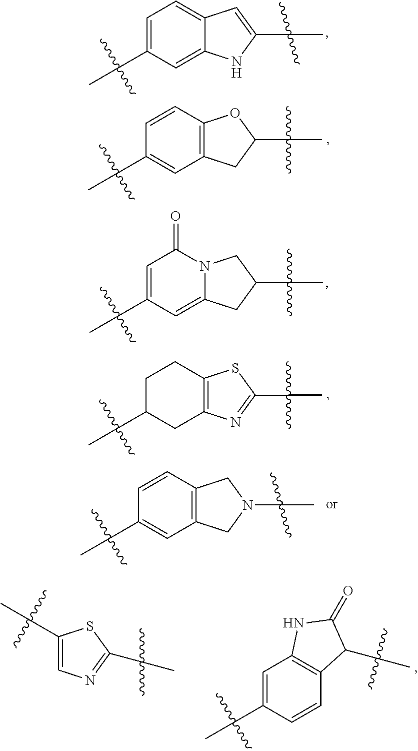

optionally substituted with 0-3 R^{y1};
R^{y1} is selected from carbonyl, hydroxyl or C₁₋₈ alkyl;
R^{y2} is selected from H; or
R^{y1} and R^{y2} together with L₁ form the following structure:

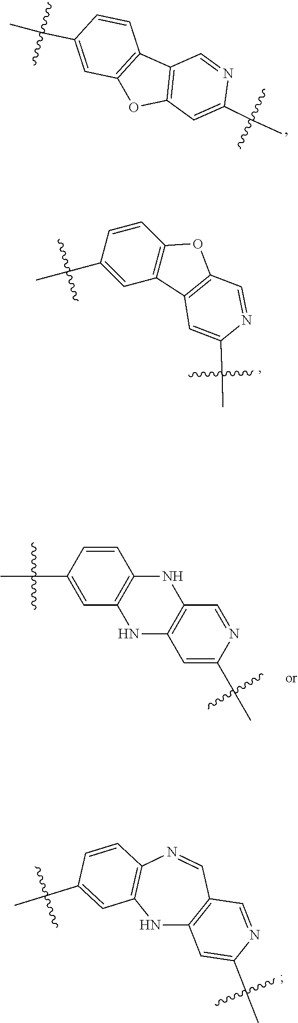 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

X₄₇ is selected from CH or N; in certain embodiments, X₄₇ is selected from N;
Y₂ is selected from phenyl,

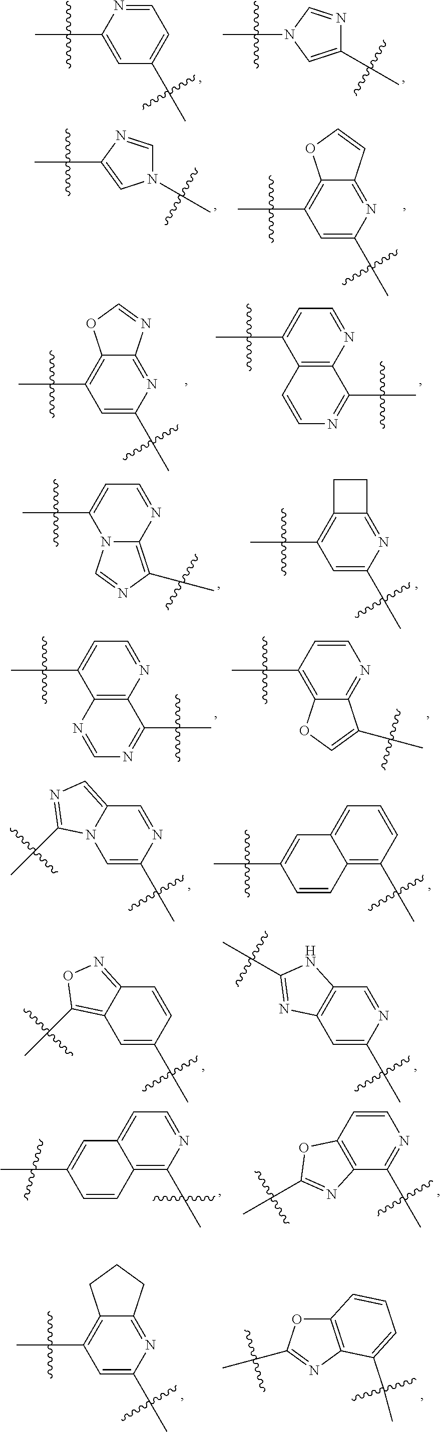

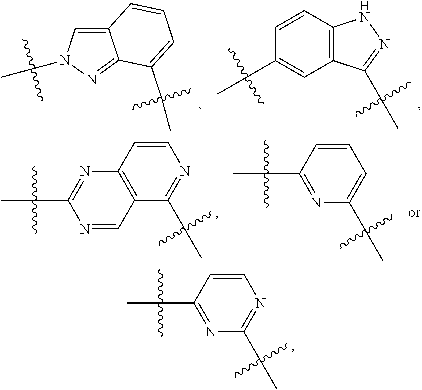

wherein the above-mentioned groups are optionally substituted with 0 to 3 of the following groups: C₁₋₈ haloalkyl, C₁₋₈ alkyl and cyano;
Y₄ is selected from cyclopropylethynyl, cyclopentyl, methoxyphenyl,

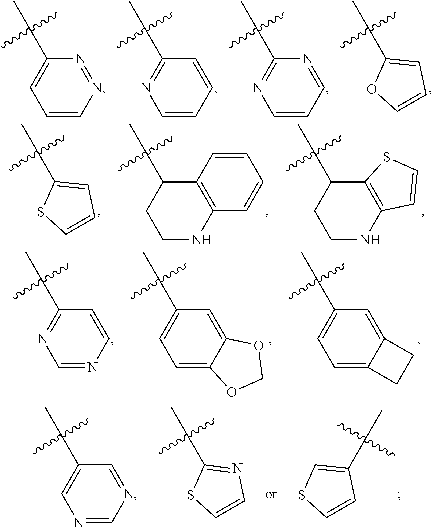

and Y₅ is selected from

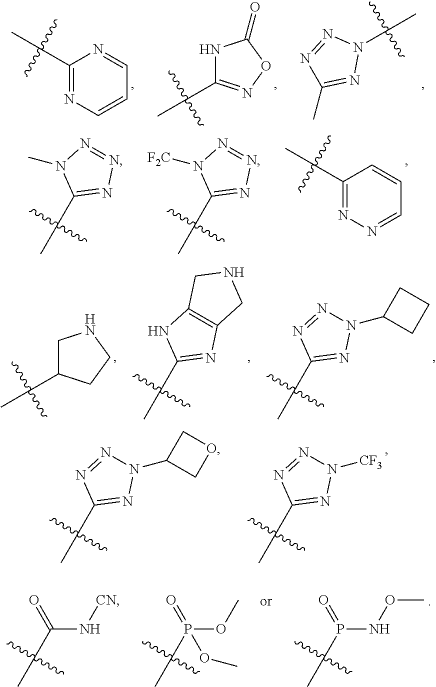

Embodiment thirteen of the present disclosure relates to a compound of formula (I-a), an isomer thereof or a pharmaceutically acceptable salt thereof, having a structure of formula (I-9),

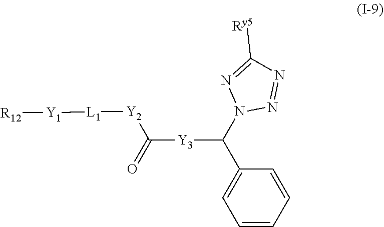

wherein R^{y5} is selected from C₁₋₃ alkyl, C₁₋₃ haloalkyl, hydroxyl-substituted C₁₋₃ alkyl or C₃₋₈ cycloalkyl;
R¹² is selected from cyano,

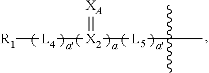

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
and L₁ is selected from a bond.

Embodiment fourteen of the present disclosure relates to a compound of formula (I-9), an isomer thereof or a pharmaceutically acceptable salt thereof, wherein Y₁ is selected from

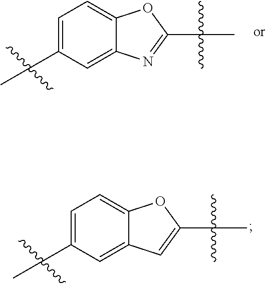 [SMILES: *c1ccc2oc(*)nc2c1 ; *c1ccc2oc(*)cc2c1]

Y₂ is selected from

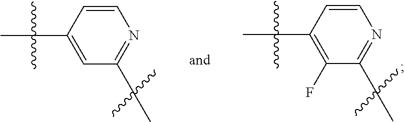 [SMILES: *c1ccnc(*)c1 ; *c1ccnc(*)c1F]

Y₃ is selected from

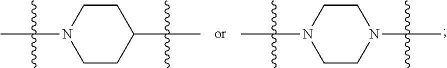 [SMILES: *C1CCN(*)CC1 ; *N1CCN(*)CC1]

R^{y}s is selected from methyl, difluoromethyl, hydroxymethyl, hydroxyethyl, cyclopropyl and cyclobutyl, and in certain embodiments, R^{y}s is selected from methyl, difluoromethyl, hydroxymethyl and cyclopropyl; and other groups are consistent with those of embodiment thirteen.

Embodiment fifteen of the present disclosure relates to a compound of formula (I-9), an isomer thereof or a pharmaceutically acceptable salt thereof, wherein R₁₂ is selected from

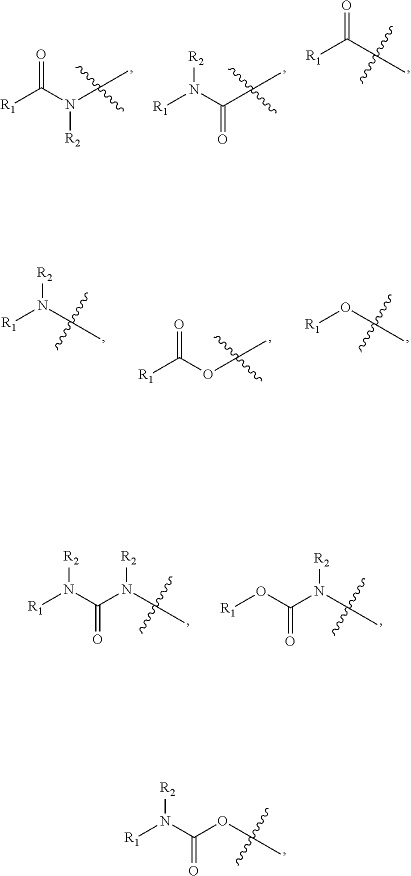

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
the definitions of other groups are consistent with those of embodiment thirteen; or
the definitions of other groups are consistent with those of embodiment fourteen.

Embodiment sixteen of the present disclosure relates to a compound of formula (I-9), an isomer thereof or a pharmaceutically acceptable salt thereof, wherein

R₁₂ is selected from pyridyl, thiazolyl, pyrazinyl, acetamido,

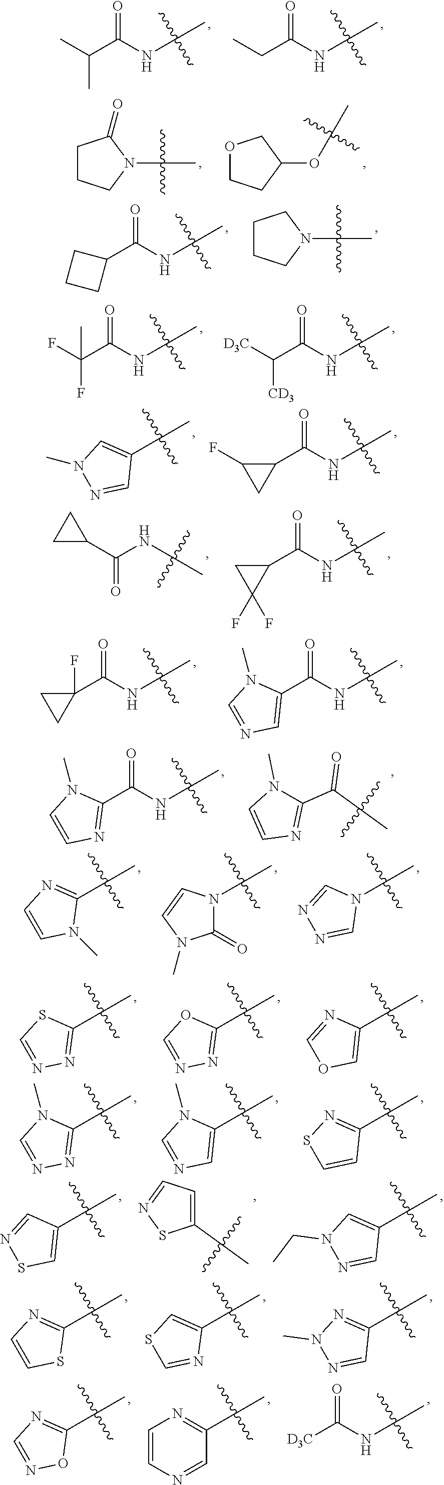

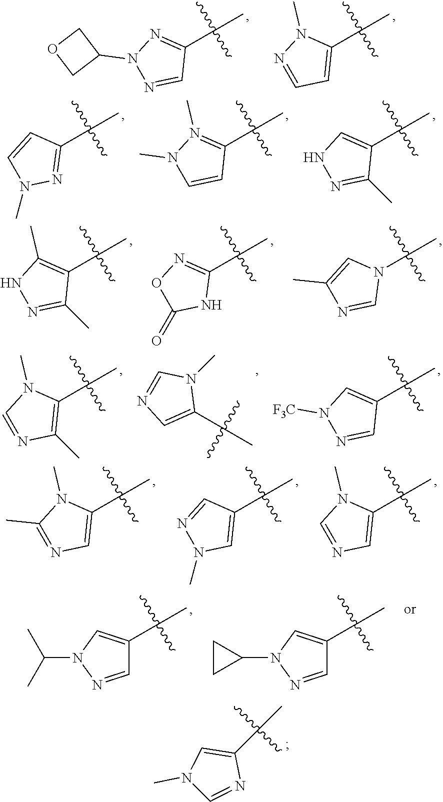

the definitions of other groups are consistent with those of embodiment thirteen; or
the definitions of other groups are consistent with those of embodiment fourteen; or
the definitions of other groups are consistent with those of embodiment fifteen.

For the compound, the isomer thereof or the pharmaceutically acceptable salt thereof of the present disclosure, L₁ is selected from a bond, ethynyl, ethenyl, azo group,

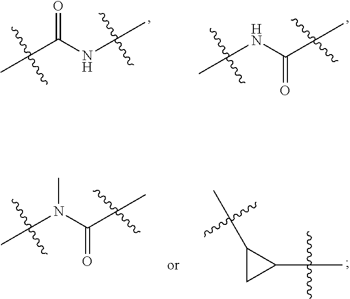

and the definitions of other groups are consistent with those of embodiment five of the present disclosure.

For the compound, the isomer thereof or the pharmaceutically acceptable salt thereof of the present disclosure, L₂ is selected from a bond, ═O, sulfonyl,

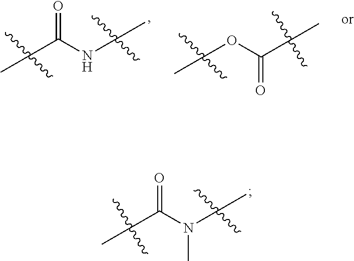

and the definitions of other groups are consistent with those of embodiment five of the present disclosure.

For the compound, the isomer thereof or the pharmaceutically acceptable salt thereof of the present disclosure, L₃ is selected from a bond, methylene, halomethylene, imino,

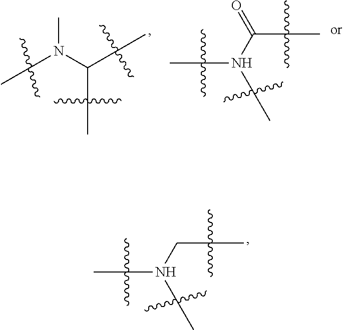

and the definitions of other groups are consistent with those of embodiment five of the present disclosure.

For the compound, the isomer thereof or the pharmaceutically acceptable salt thereof of the present disclosure,

L₁ is selected from a bond, ethynyl, ethenyl, azo group,

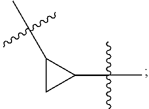 [SMILES: *C1CC1*]

L₂ is selected from a bond, ═O, sulfonyl,

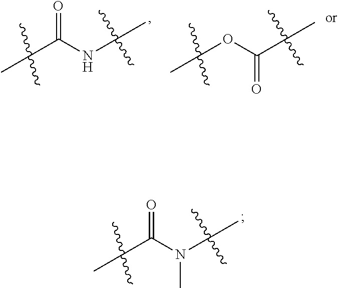

L₃ is selected from a bond, methylene, halomethylene, imino,

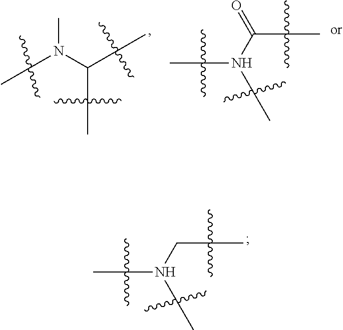

and the definitions of other groups are consistent with those of embodiment five of the present disclosure.

For the compound, the isomer thereof or the pharmaceutically acceptable salt thereof of the present disclosure,

L₁ is selected from a bond; L₂ is selected from carbonyl; L₃ is selected from

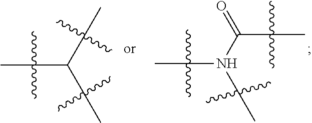

and the definitions of other groups are consistent with those of embodiment five of the present disclosure.
For the compound, the isomer thereof or the pharmaceutically acceptable salt thereof of the present disclosure,
L₁ is selected from a bond; L₂ is selected from carbonyl; L₃ is selected from

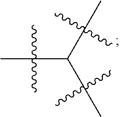 [SMILES: *C(*)*]

and the definitions of other groups are consistent with those of embodiment five of the present disclosure.

For the compound, the isomer thereof or the pharmaceutically acceptable salt thereof of the present disclosure,

Y₁ is selected from phenyl,

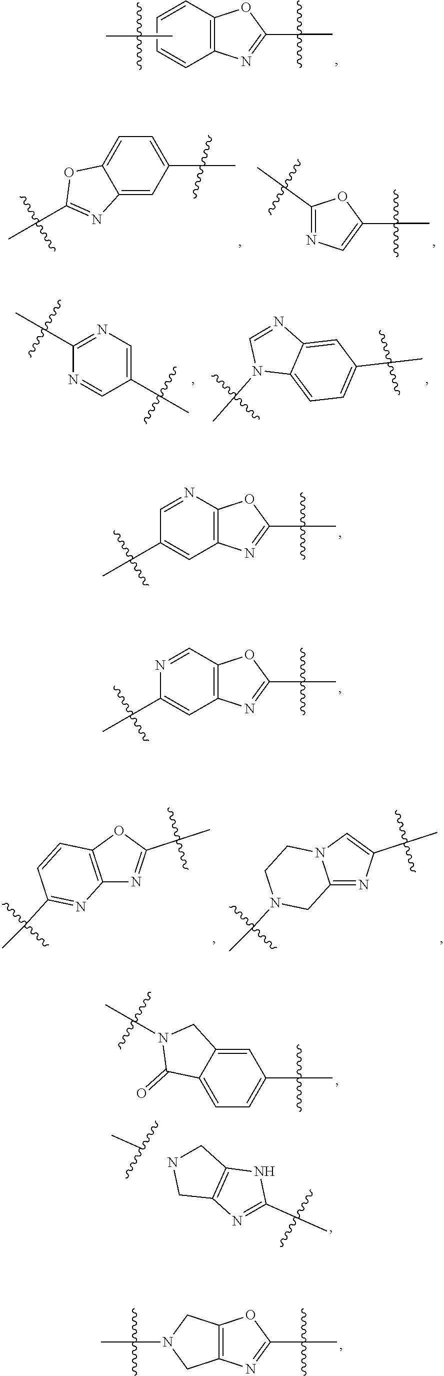

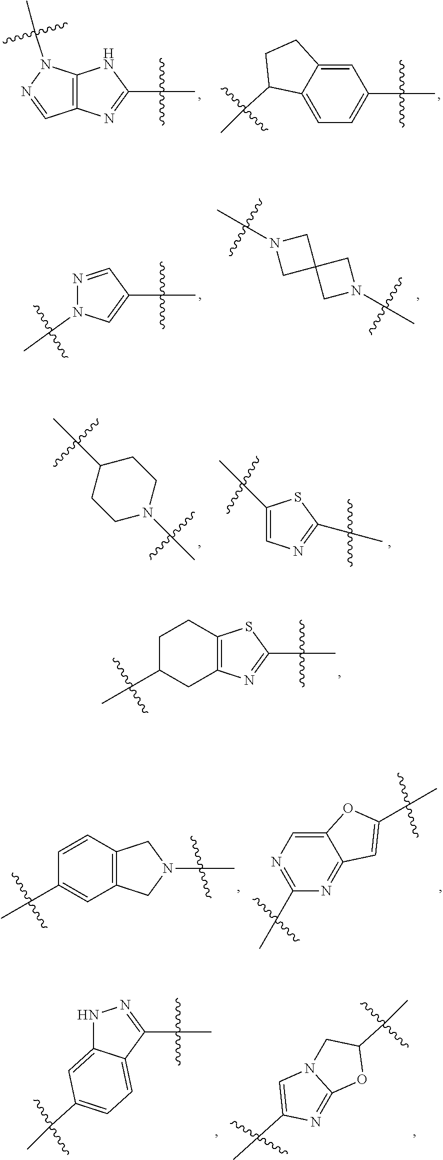

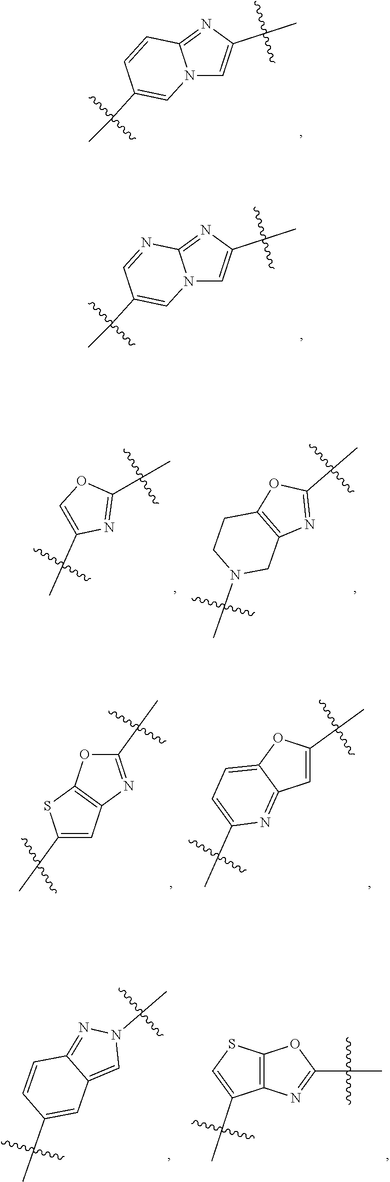

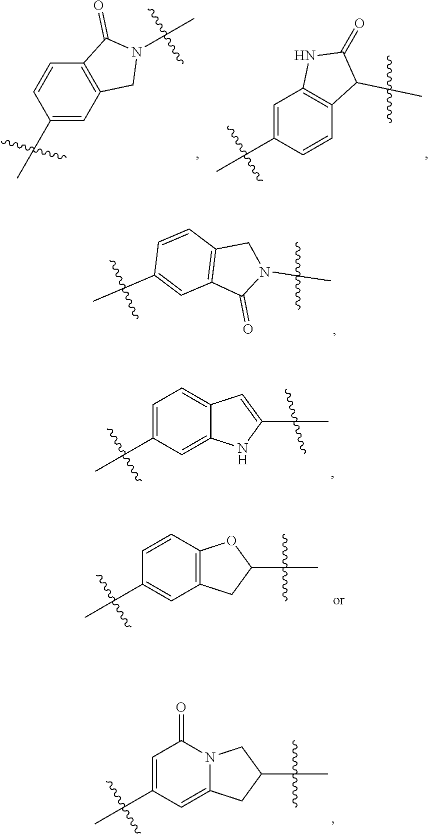 [SMILES: *c1ccc2c(c1)CN(*)C2=O ; *c1ccc2c(c1)NC(=O)C2* ; *c1ccc2c(c1)C(=O)N(*)C2 ; *c1ccc2cc(*)[nH]c2c1 ; *c1ccc2c(c1)CC(*)O2 ; *c1cc2n(c(=O)c1)CC(*)C2]

wherein the above-mentioned groups are optionally substituted with 0-5 R^{y1}, and the definitions of other groups are consistent with those of embodiment five of the present disclosure.

For the compound, the isomer thereof or the pharmaceutically acceptable salt thereof of the present disclosure, Y₁ is selected from phenyl and

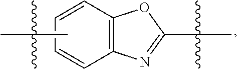

definitions of other groups are consistent with those of embodiment five of the present disclosure.

For the compound, the isomer thereof or the pharmaceutically acceptable salt thereof of the present disclosure, Y₁ is selected from

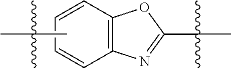

and optionally substituted with 0-5 R^{y1}, and the definitions of other groups are consistent with those of embodiment five of the present disclosure.

For the compound, the isomer thereof or the pharmaceutically acceptable salt thereof of the present disclosure, Y₁ is selected from

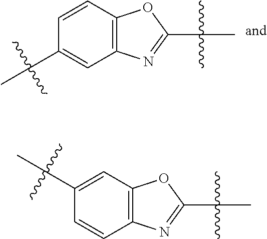 [SMILES: *c1ccc2oc(*)nc2c1 ; *c1ccc2nc(*)oc2c1]

and optionally substituted with 0-5 R^{y1}, and the definitions of other groups are consistent with those of embodiment five of the present disclosure.

For the compound, the isomer thereof or the pharmaceutically acceptable salt thereof of the present disclosure, R¹² is selected from acetyl, acetamido, difluoromethyloxy, cyano, pyridyl, thiazolyl, pyrrolidinyl, pyrazinyl,

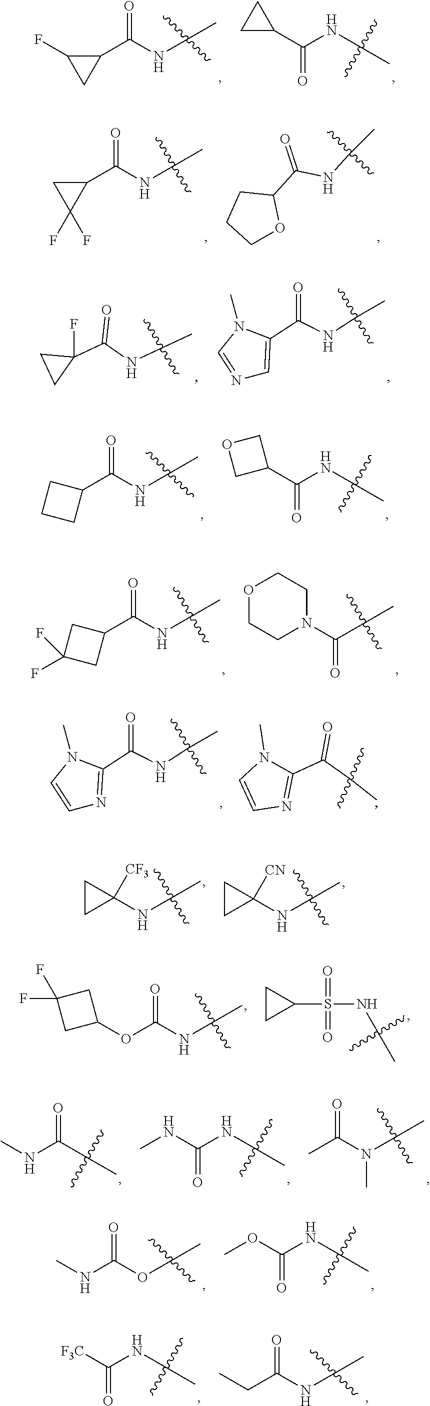

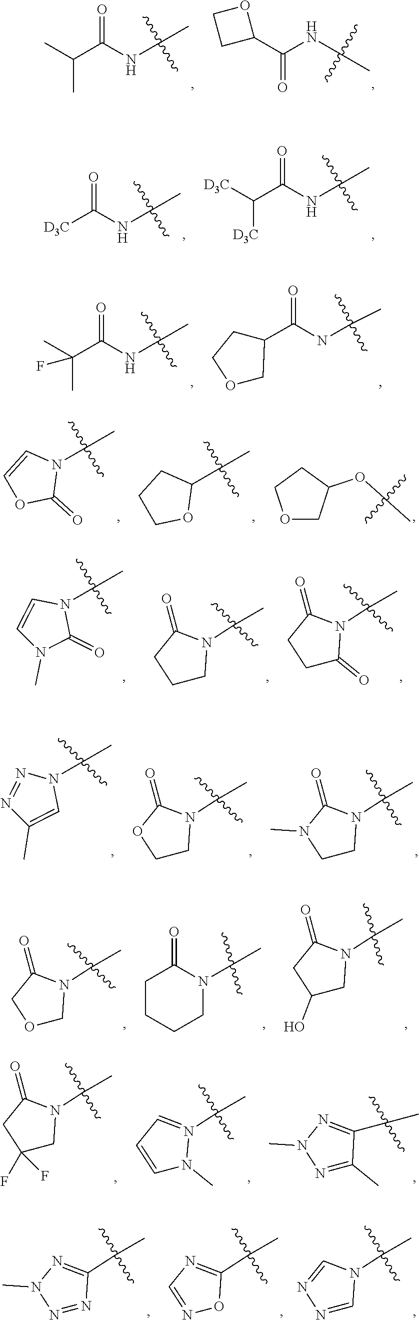

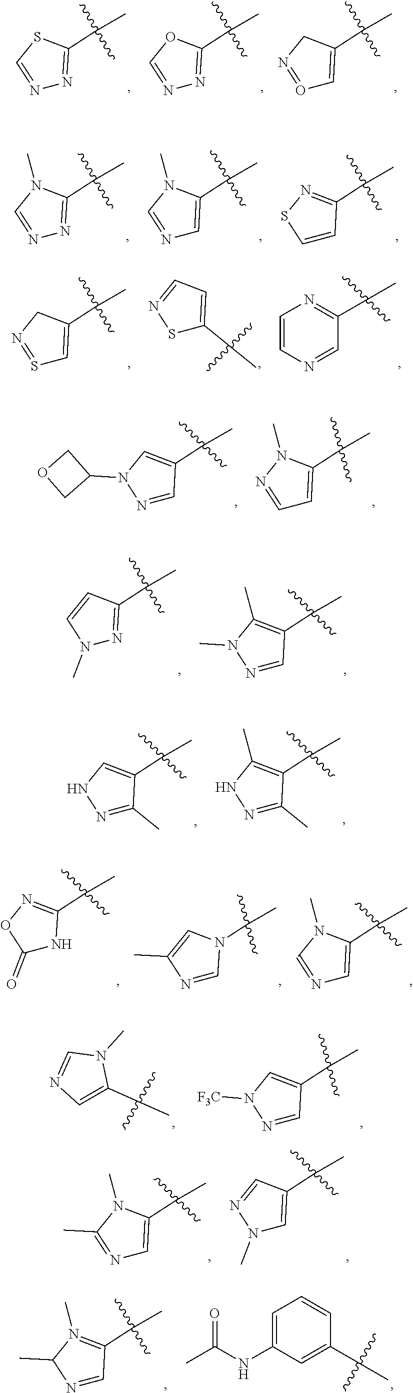

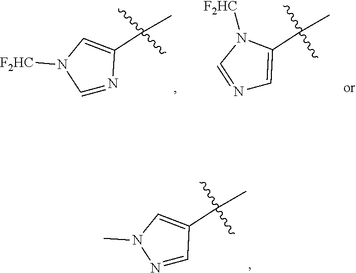

and the definitions of other groups are consistent with those of embodiment five of the present disclosure.

For the compound, the isomer thereof or the pharmaceutically acceptable salt thereof of the present disclosure, R₁₂ is selected from pyridyl, thiazolyl, pyrazinyl, acetamido,

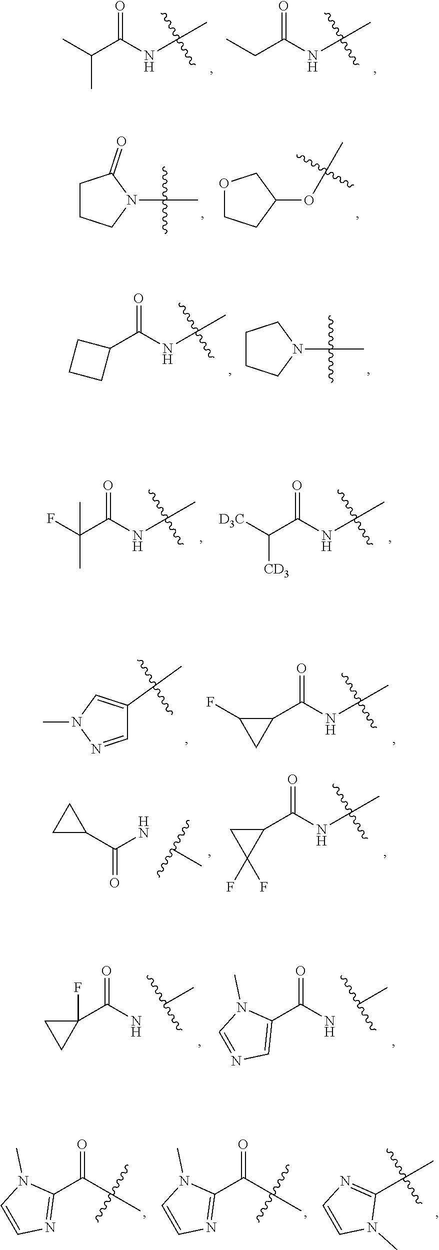

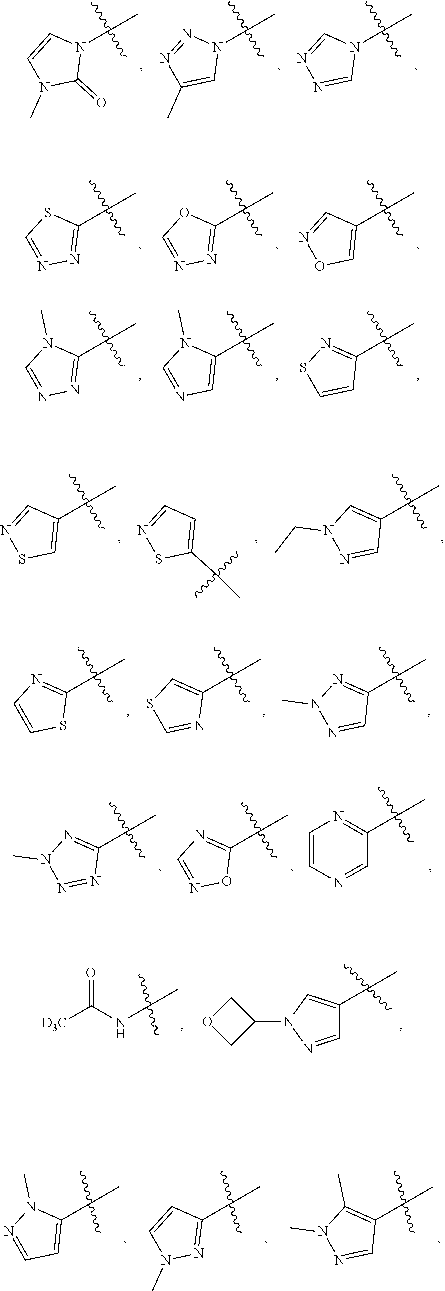

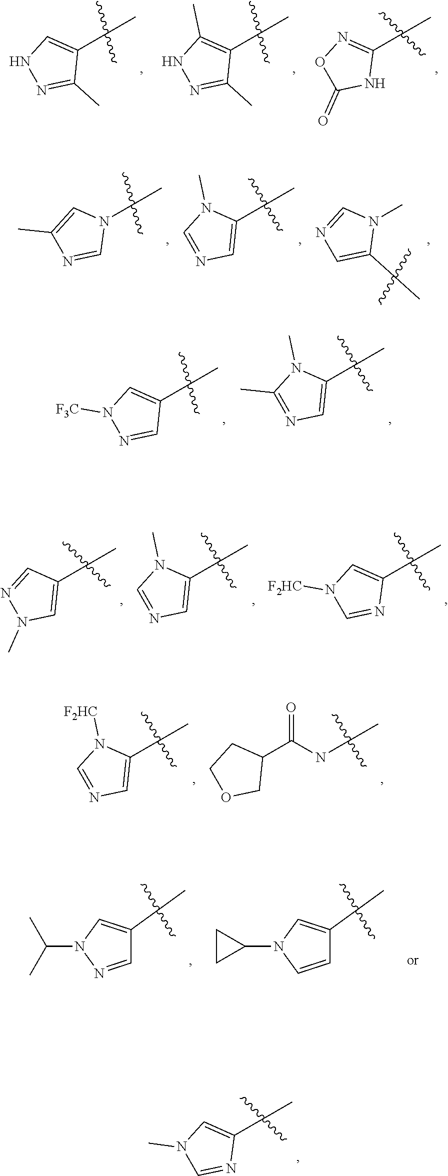

and the definitions of other groups are consistent with those of embodiment five of the present disclosure.

For the compound, the isomer thereof or the pharmaceutically acceptable salt thereof of the present disclosure, R¹² is selected from acetyl, acetamido, difluoromethyloxy, pyrrolidinyl,

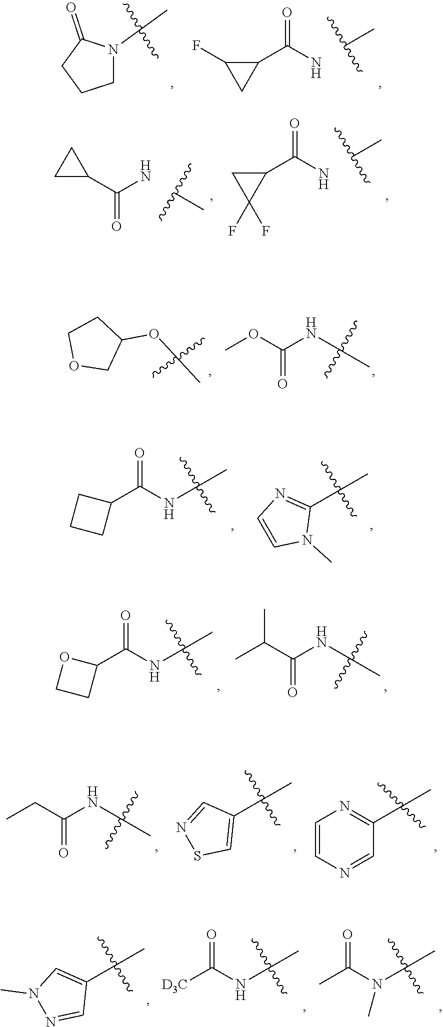

and the definitions of other groups are consistent with those of embodiment five of the present disclosure.

For the compound, the isomer thereof or the pharmaceutically acceptable salt thereof of the present disclosure, R¹² is selected from acetyl, acetamido, difluoromethyloxy, pyrrolidinyl,

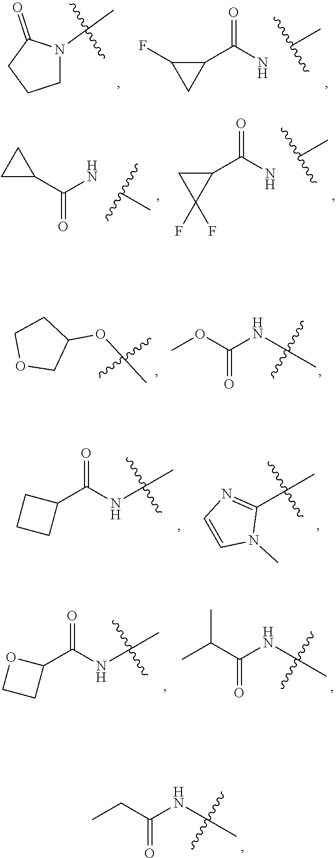

and the definitions of other groups are consistent with those of embodiment five of the present disclosure.

For the compound, the isomer thereof or the pharmaceutically acceptable salt thereof of the present disclosure, R¹² is selected from acetyl, acetamido, difluoromethyloxy, pyrrolidinyl,

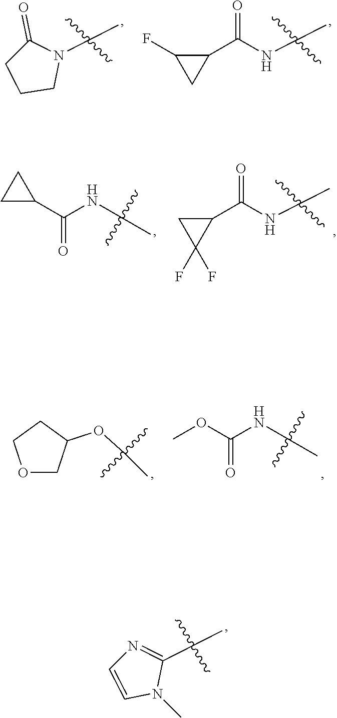

and the definitions of other groups are consistent with those of embodiment five of the present disclosure.

For the compound, the isomer thereof or the pharmaceutically acceptable salt thereof of the present disclosure, R¹² is selected from

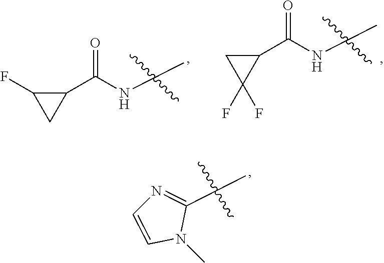

and the definitions of other groups are consistent with those of embodiment five of the present disclosure.

For the compound, the isomer thereof or the pharmaceutically acceptable salt thereof of the present disclosure, Y₃ is selected from

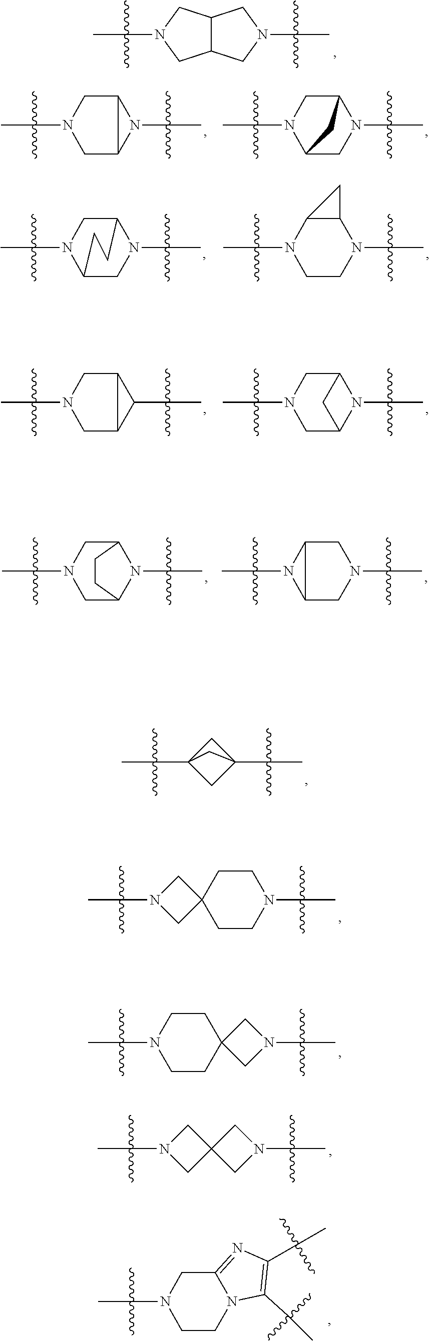

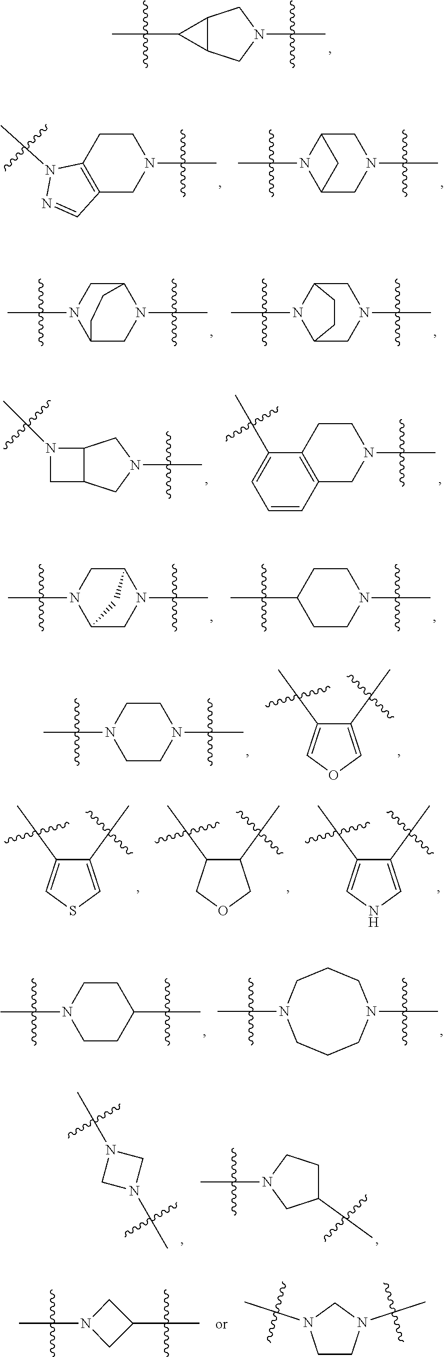

and optionally substituted with 0-3 R³; and the definitions of other groups are consistent with those of embodiment five of the present disclosure.

For the compound, the isomer thereof or the pharmaceutically acceptable salt thereof of the present disclosure, R^{y3} is selected from halogen, hydroxyl, C₁₋₈ alkyl and ═O, or together with the atom on a ring form cyclopropyl; and the definitions of other groups are consistent with those of embodiment five of the present disclosure.

For the compound, the isomer thereof or the pharmaceutically acceptable salt thereof of the present disclosure, Y₃ is selected from

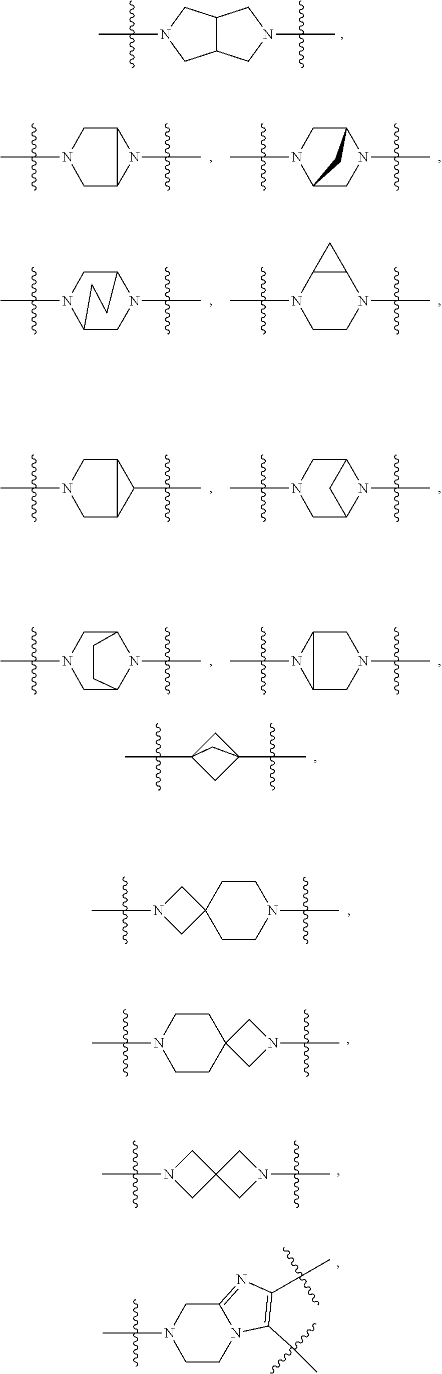

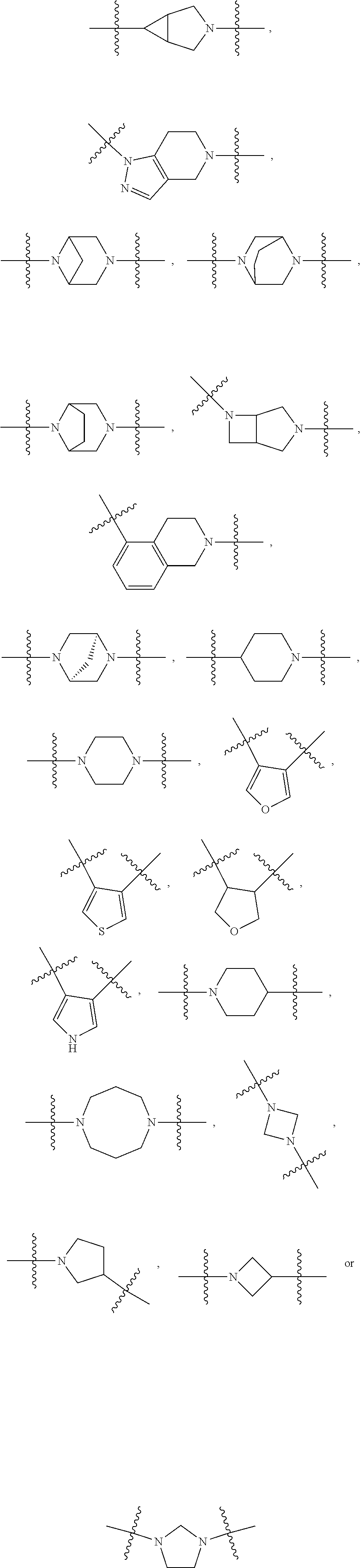

optionally substituted with 0-3 R^{y3};
R^{y3} is selected from halogen, hydroxyl, C₁₋₈ alkyl and ═O, or together with the atom on a ring form cyclopropyl;
and the definitions of other groups are consistent with those of embodiment five of the present disclosure.

For the compound, the isomer thereof or the pharmaceutically acceptable salt thereof of the present disclosure, Y₃ is selected from

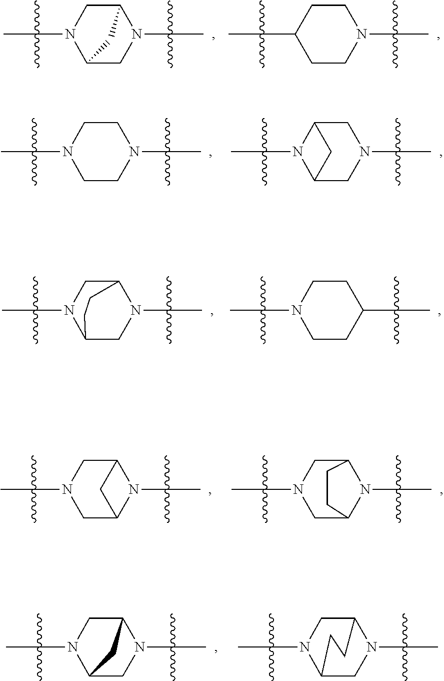

and optionally substituted with 0-3 R^{y3};
R^{y3} is selected from halogen, hydroxyl, C₁₋₈ alkyl and ═O, or together with the atom on a ring form cyclopropyl;
and the definitions of other groups are consistent with those of embodiment five of the present disclosure.

For the compound, the isomer thereof or the pharmaceutically acceptable salt thereof of the present disclosure, Y₃ is selected from

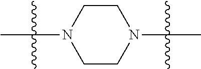 [SMILES: *N1CCN(*)CC1]

and optionally substituted with 0-3 R^{y3};
R^{y3} is selected from halogen, hydroxyl, C₁₋₈ alkyl and ═O, or together with the atom on a ring form cyclopropyl;
and the definitions of other groups are consistent with those of embodiment five of the present disclosure.

For the compound, the isomer thereof or the pharmaceutically acceptable salt thereof of the present disclosure, Y₂ is selected from phenyl,

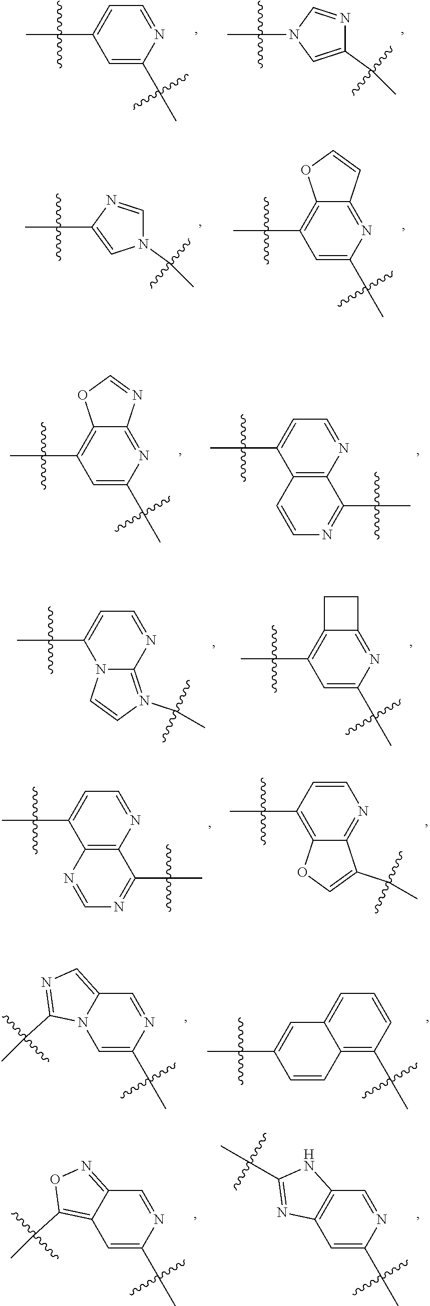

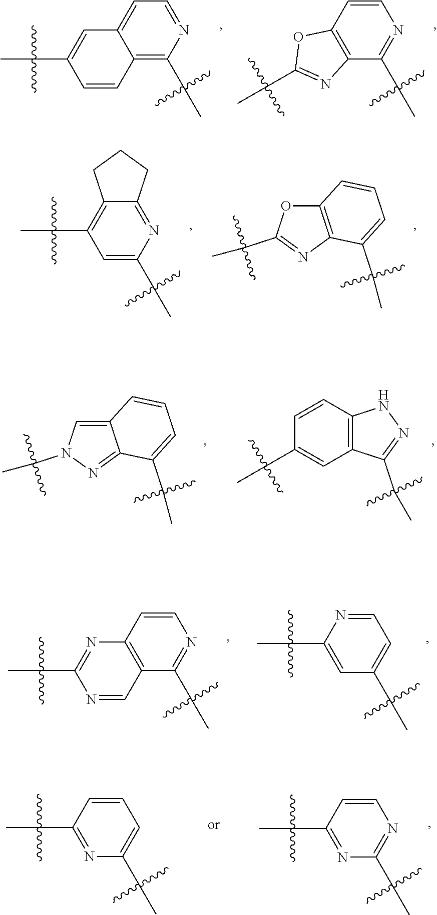

wherein the above-mentioned groups are optionally substituted with 0-3 R^{y2}; and the definitions of other groups are consistent with those of embodiment five of the present disclosure.

For the compound, the isomer thereof or the pharmaceutically acceptable salt thereof of the present disclosure, R^{y2} is selected from cyano, halogen, C₁₋₈ haloalkyl and C₁₋₈ alkyl, preferably cyano, F, trifluoromethyl, difluoromethyl and methyl; and the definitions of other groups are consistent with those of embodiment five of the present disclosure.

For the compound, the isomer thereof or the pharmaceutically acceptable salt thereof of the present disclosure,

Y₂ is selected from phenyl,

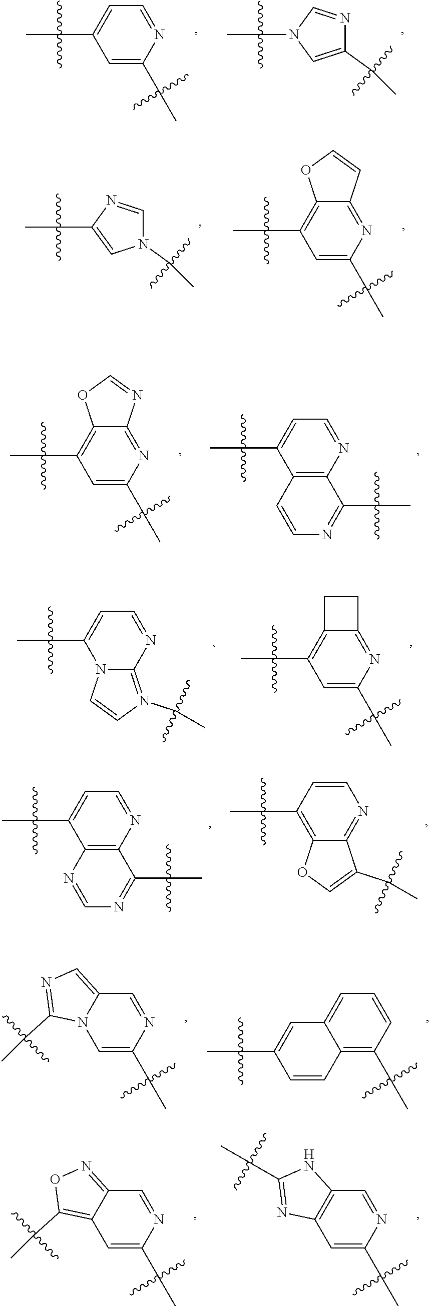

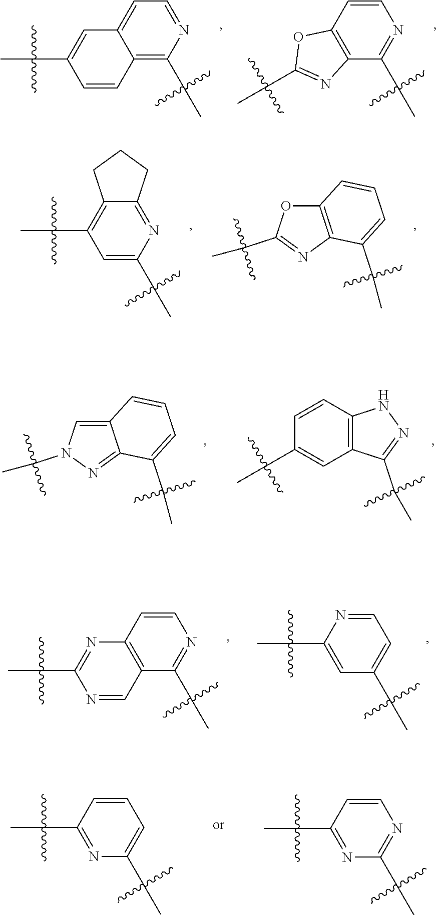

optionally substituted with 0-3 R^{y2};
R^{y2} is selected from cyano, halogen, C₁₋₈ haloalkyl and C₁₋₈ alkyl, preferably cyano, F, trifluoromethyl, difluoromethyl and methyl; and the definitions of other groups are consistent with those of embodiment five of the present disclosure.

For the compound, the isomer thereof or the pharmaceutically acceptable salt thereof of the present disclosure,

Y₂ is selected from phenyl and

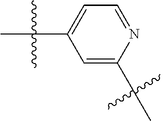 [SMILES: *c1ccnc(*)c1]

and optionally substituted with 0-3 R^{y2}; and the definitions of other groups are consistent with those of embodiment five of the present disclosure.

For the compound, the isomer thereof or the pharmaceutically acceptable salt thereof of the present disclosure, R^{y2} is selected from cyano, halogen, C₁₋₈ haloalkyl and C₁₋₈ alkyl, preferably cyano, F, trifluoromethyl, difluoromethyl and methyl; and the definitions of other groups are consistent with those of embodiment five of the present disclosure.

For the compound, the isomer thereof or the pharmaceutically acceptable salt thereof of the present disclosure,

Y₂ is selected from phenyl and

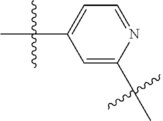 [SMILES: *c1ccnc(*)c1]

and optionally substituted with 0-3 R^{y2}; R^{y2} is selected from cyano, halogen, C₁₋₈ haloalkyl and C₁₋₈ alkyl, preferably cyano, F, trifluoromethyl, difluoromethyl and methyl; and the definitions of other groups are consistent with those of embodiment five of the present disclosure.

For the compound, the isomer thereof or the pharmaceutically acceptable salt thereof of the present disclosure, Y₄ is selected from phenyl, ethynyl, cyclopentyl,

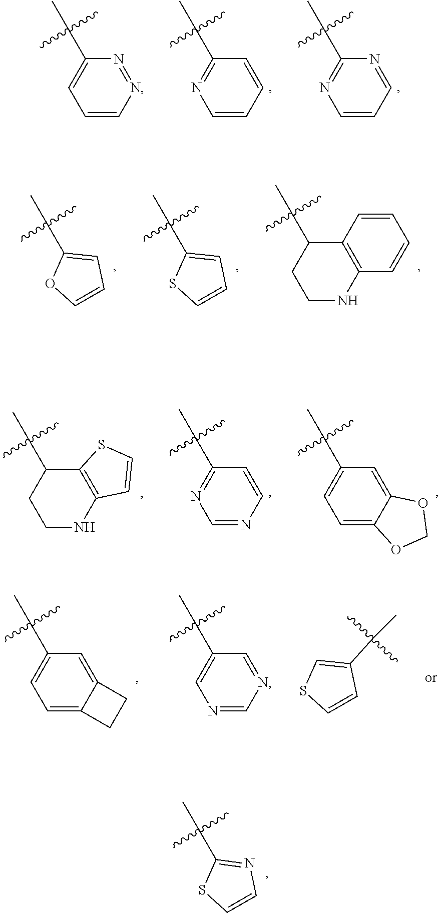

wherein the above-mentioned groups are optionally substituted with 0-3 R^{y4}; and the definitions of other groups are consistent with those of embodiment five of the present disclosure.

For the compound, the isomer thereof or the pharmaceutically acceptable salt thereof of the present disclosure, R^{y4} is selected from halogen, cyano, C₅₋₁₀ aryl, C₁₋₈ haloalkyl, C₁₋₈ alkoxy and C₃₋₈ cycloalkyl; in certain embodiments, R^{y4} is selected from F, cyano, phenyl, trifluoromethyl, methoxy and cyclopropyl; and the definitions of other groups are consistent with those of embodiment five of the present disclosure.

For the compound, the isomer thereof or the pharmaceutically acceptable salt thereof of the present disclosure, Y₄ is selected from phenyl, ethynyl, cyclopentyl,

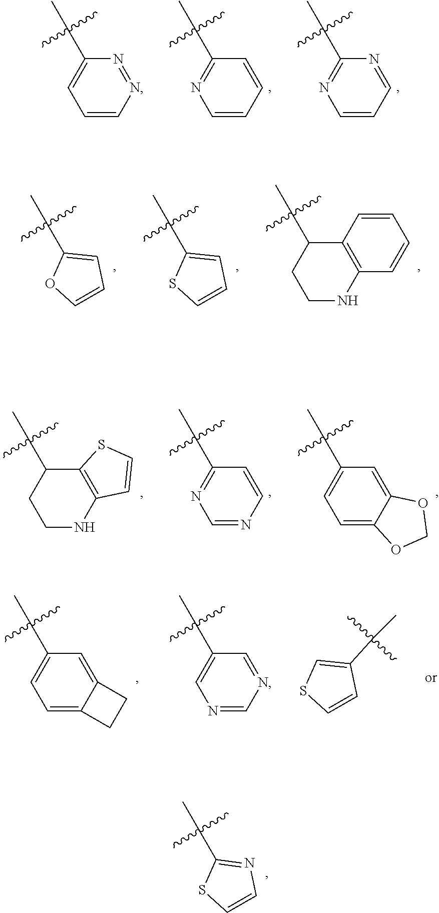

wherein the above-mentioned groups are optionally substituted with 0-3 R^{y4}; R^{y4} is selected from halogen, cyano, C₅₋₁₀ aryl, C₁₋₈ haloalkyl, C₁₋₈ alkoxy and C₃₋₈ cycloalkyl; in certain embodiments, R^{y4} is selected from F, cyano, phenyl, trifluoromethyl, methoxy and cyclopropyl; and the definitions of other groups are consistent with those of embodiment five of the present disclosure.

For the compound, the isomer thereof or the pharmaceutically acceptable salt thereof of the present disclosure, Y₄ is selected from phenyl, wherein the phenyl is optionally substituted with 0-3 R^{y4};

R^{y4} is selected from halogen, cyano, C₅₋₁₀ aryl, C₁₋₈ haloalkyl, C₁₋₈ alkoxy and C₃₋₈ cycloalkyl; in certain embodiments, R^{y4} is selected from F, cyano, phenyl, trifluoromethyl, methoxy and cyclopropyl; and the definitions of other groups are consistent with those of embodiment five of the present disclosure.

For the compound, the isomer thereof or the pharmaceutically acceptable salt thereof of the present disclosure, Y₅ is selected from

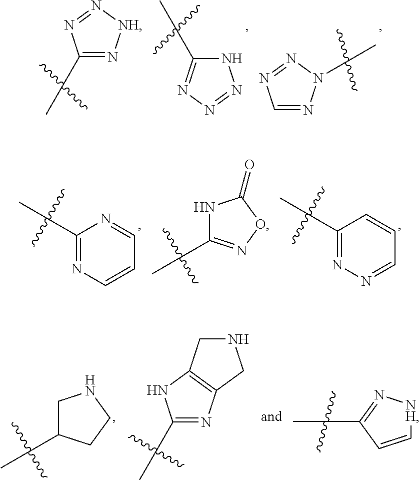

wherein the above-mentioned groups are optionally substituted with 0-3 R⁵; and the definitions of other groups are consistent with those of embodiment five of the present disclosure.

R^{y5} is selected from C₁₋₈ alkyl, C₁₋₈ haloalkyl, C₃₋₈ cycloalkyl and 3-8 membered heterocyclyl, preferably methyl, cyclopropyl, cyclobutyl, trifluoromethyl, difluoromethyl, fluoroethyl and oxacyclobutyl; and the definitions of other groups are consistent with those of embodiment five of the present disclosure.

For the compound, the isomer thereof or the pharmaceutically acceptable salt thereof of the present disclosure, Y₅ is selected from

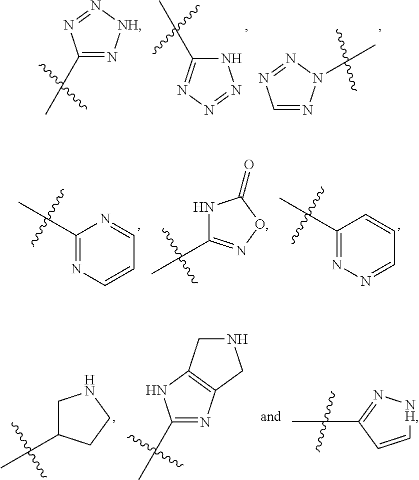

wherein the above-mentioned groups are optionally substituted with 0-3 R^{y5}; R^{y5} is selected from C₁₋₈ alkyl, C₁₋₈ haloalkyl, C₃₋₈ cycloalkyl and 3-8 membered heterocyclyl, preferably methyl, cyclopropyl, cyclobutyl, trifluoromethyl, difluoromethyl, fluoroethyl and oxacyclobutyl; and the definitions of other groups are consistent with those of embodiment five of the present disclosure.

Embodiment seventeen of the present disclosure relates to a compound represented by general formula (I-a), an isomer thereof or a pharmaceutically acceptable salt thereof,

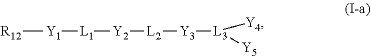

wherein:
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally further substituted with 0-5 R^{y1};
Y₂ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y2};
Y₃ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y3};
Y₄ is selected from C₃₋₁₂ carbocyclyl, C₅₋₁₅ aryl, 5-15 membered heteroaryl, 3-12 membered heterocyclyl, C₂₋₈ alkenyl and C₂₋₈ alkynyl, wherein the carbocyclyl, aryl, heteroaryl, heterocyclyl, alkenyl and alkynyl are optionally substituted with 0-5 R^{y4};
Y₅ is selected from C₃₋₁₂ carbocyclyl, C₅₋₁₅ aryl, 5-15 membered heteroaryl, 3-12 membered heterocyclyl, —C(O)NHOCH₃, —C(O)NHCN, —P(O)(OCH₃)₂ and —C(O)OCH₂CH₃, wherein the carbocyclyl, aryl, heteroaryl and heterocyclyl are optionally substituted with 0-5 R^{y5};
R^{y1}, R^{y2}, R^{y3}, R^{y4} and R^{y5} are each independently selected from deuterium, halogen, ═O, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ hydroxylalkyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl, 5-10 membered heteroaryl or acetoxy; or
R^{y3} and the atom on Y₃ ring form C₃₋₈ cycloalkyl; or
R^{y1} and R^{y2} together with L₁ form 4-8 membered heterocyclyl, C₄₋₈ carbocyclyl or 5-8 membered heteroaryl;
R₁₂ is selected from H, cyano, C₁₋₈ alkoxy, C₃₋₈ cycloalkyl,

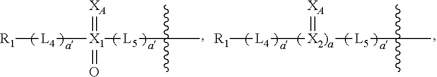

3-8 membered heterocyclyl, C₅₋₁₀ aryl or 5-10 membered heteroaryl, wherein the cycloalkyl, heterocyclyl, aryl or heteroaryl is optionally further substituted with 0-5 of the following groups: halogen, ═O, acetamido, hydroxyl, cyano, sulfhydryl, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₁ is selected from P or S;
X₂ is selected from C, PR₁ₐ or S;
L₄ and L₅ are each independently selected from: a bond, NR₂, CR₃R₄, O, or S;
X_{A} is selected from C, NR₂ₐ, O and S; a is 0 or 1; a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, C₁₋₈ alkyl, cyano, ═O, C₁₋₈ alkoxy or C₁₋₈ haloalkyl;
R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: halogen, C₁₋₈ alkyl, C₁₋₈ alkoxy or C₁₋₈ haloalkyl;
L₁ is selected from a bond, —(CR₆)ₜ—NR₅—(CR₇)_{t′}—, C₂₋₈ alkynyl, C₂₋₈ alkenyl,

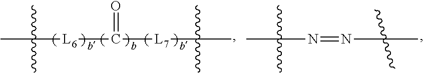

3-8 membered heterocyclyl, C₃₋₈ cycloalkyl, C₅₋₁₀ aryl or 5-10 membered heteroaryl, wherein the alkynyl, alkenyl, heterocyclyl, cycloalkyl, aryl or heteroaryl is optionally substituted with 0-5 groups selected from: hydroxyl, halogen, C₁₋₈ alkyl or C₁₋₈ haloalkyl;
t and t′ are selected from 0, 1, 2 and 3;
L₂ is selected from a bond,

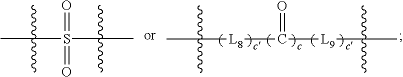

L₆, L₇, L₈ and L₉ are each independently selected from a bond, O, S, NR₅ or CR₆R₇; b and c are each independently selected from 0 or 1; b′ and c′ are each independently selected from 0, 1, 2, 3, 4, 5 or 6; R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl;
L₃ is selected from a bond,

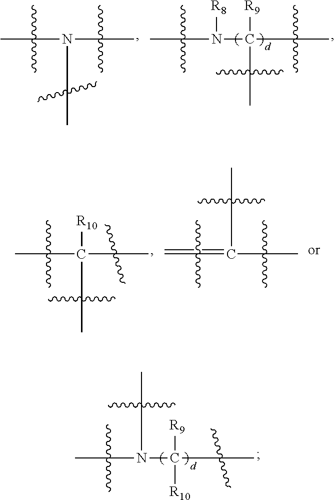

d is an integer selected from 1-6; and R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl;
provided that:
L₁, L₂ and L₃ are not simultaneously a bond;
when Y₁ is selected from

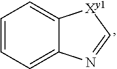

X^{y1} is O or S, L₁ is a bond, L₂ is carbonyl, L₃ is

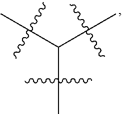 [SMILES: *C(*)*]

Y₂ is selected from

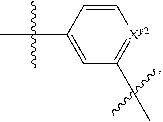

X^{y2} is C or N, Y₃ is selected from

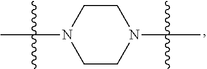 [SMILES: *N1CCN(*)CC1]

Y₄ is selected from a benzene ring, Y₁ is substituted with 0 R^{y1}, and R₁₂ is selected from acetamido, halogen, methyl, methoxy, trifluoromethyl, trifluoromethyloxy, hydroxyl, methanesulfonylamido, methylamino or N,N-dimethylamino, Y₅ is not selected from the following groups:

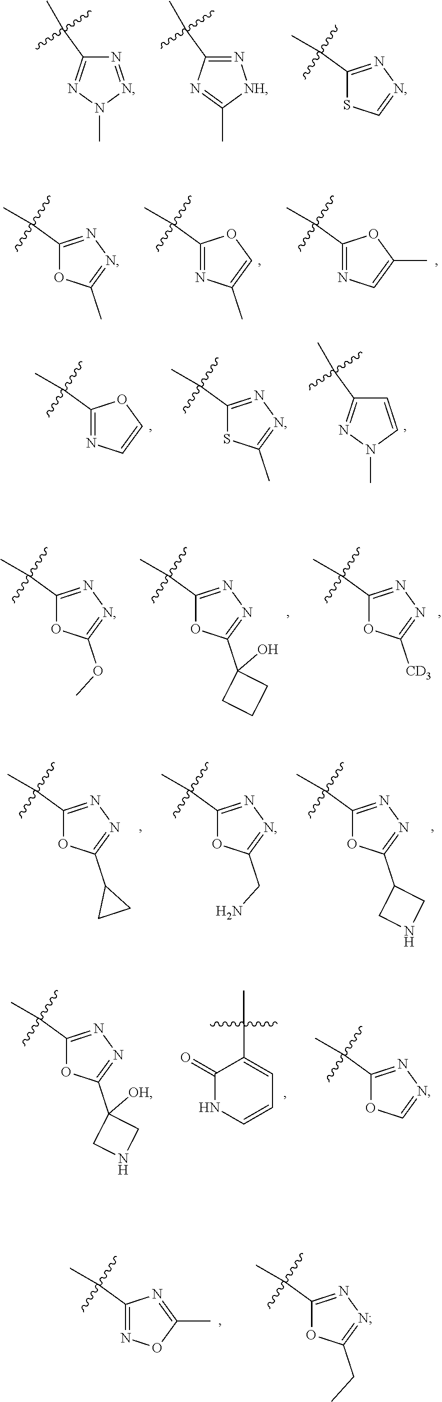

when R₁₂—Y₁ is selected from

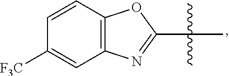 [SMILES: *c1nc2cc(C(F)(F)F)ccc2o1]

L₁ is a bond, L₂ is carbonyl, L₃ is

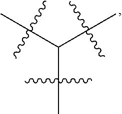 [SMILES: *C(*)*]

Y₃ is selected from

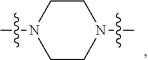

Y₄ is selected from a benzene ring and Y₅ is selected from

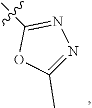

Y₂ is not selected from the following groups:

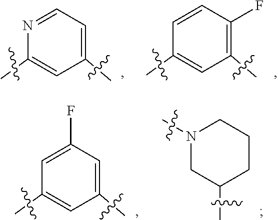

when Y₅ is selected from acetamido, L₃ is a bond;
and when Y₅ is —C(O)OCH₂CH₃, it does not have the following structure:

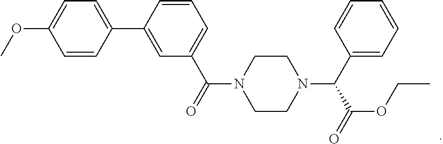 [SMILES: CCOC(=O)[C@@H](c1ccccc1)N1CCN(C(=O)c2cccc(-c3ccc(OC)cc3)c2)CC1]

Embodiment eighteen of the present disclosure relates to a compound of formula (I-a), an isomer thereof or a pharmaceutically acceptable salt thereof, having a structure of formula (I-1),

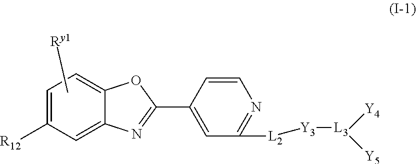

Y₃ is selected from

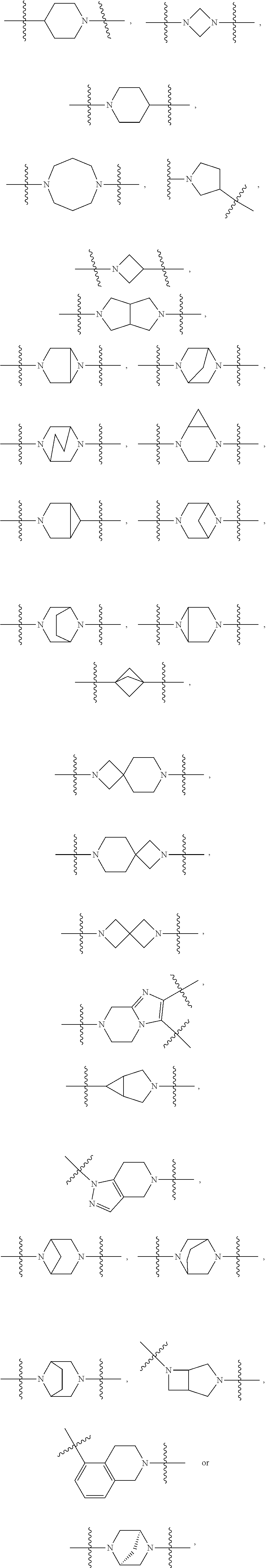

wherein the above-mentioned groups are optionally further substituted with 0 to 3 of the following groups: halogen, hydroxyl, cyano, C₁₋₈ alkyl or C₁₋₈ haloalkyl;
Y₄ is selected from phenyl;
Y₅ is selected from

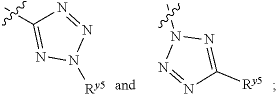

R^{y5} is selected from methyl or fluoroethyl;
L₂ is selected from carbonyl;
L₃ is selected from

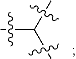

R₁₂ is selected from acetamido, cyclopropylacetamido, fluorocyclopropylacetamido or

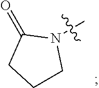

and R^{y1} is selected from H.

In certain embodiments, for the formula (I-1) of embodiment eighteen, Y₃ is selected from

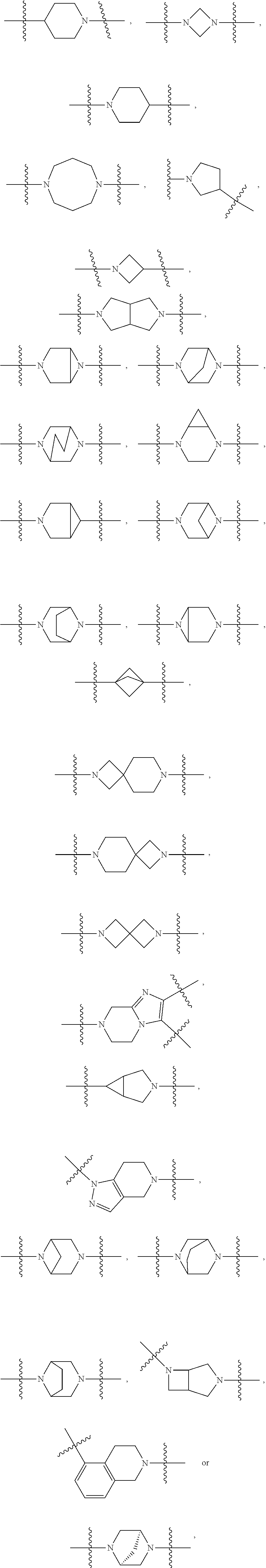

wherein the above-mentioned groups are optionally further substituted with 0 to 3 of the following groups: halogen, hydroxyl, cyano, C₁₋₈ alkyl or C₁₋₈ haloalkyl.

Embodiment nineteen of the present disclosure relates to a compound of formula (I-a), an isomer thereof or a pharmaceutically acceptable salt thereof, wherein

Y₄ is selected from phenyl optionally substituted with 0-5 R^{y4};
Y₅ is selected from tetrazolyl

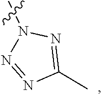

wherein the tetrazolyl is further substituted with monofluoromethyl, difluoromethyl, fluoroethyl, hydroxymethyl, hydroxyethyl and methoxymethyl, preferably substituted with difluoromethyl, fluoroethyl and hydroxymethyl;
and the definitions of other substituents are consistent with those of embodiment seventeen.

Embodiment twenty of the present disclosure relates to a compound of formula (I-a), an isomer thereof or a pharmaceutically acceptable salt thereof, which has a structure of formula (I-2),

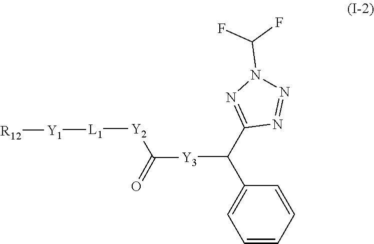

wherein,
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R₁₂ is selected from cyano,

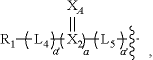

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R^{y1} is selected from halogen, cyano and C₁₋₈ haloalkyl; or
R^{y1} and R^{y2} together with L₁ form 4-8 membered heterocyclyl, C₄₋₈ carbocyclyl or 5-8 membered heteroaryl;
X₂ is selected from C;
L₄ and L₅ are each independently selected from: a bond, NR₂, CR₃R₄, O, or S;
X_{A} is selected from O and S; a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
R₂, R₃ and R₄ are each independently selected from: H, halogen, C₁₋₈ alkyl or C₁₋₈ haloalkyl;
and the definitions of other substituents are consistent with those of embodiment seventeen or nineteen.

Embodiment twenty-one of the present disclosure relates to a compound of formula (I-2), an isomer thereof or a pharmaceutically acceptable salt thereof, wherein

Y₃ is selected from

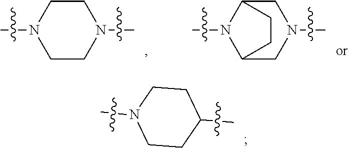

and/or
Y₂ is selected from

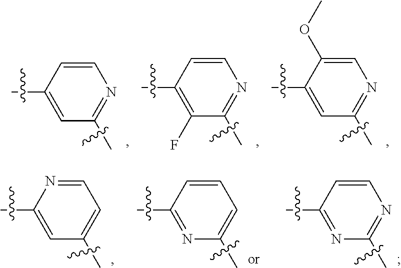

and/or
and L₁ is selected from a bond,

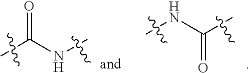

and the definitions of other substituents are consistent with those of embodiment seventeen, nineteen or twenty-one.

For the compound of formula (I-2), the isomer thereof or the pharmaceutically acceptable salt thereof of the present disclosure, Y₃ is selected from

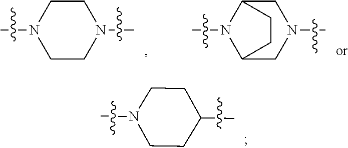

Y₂ is selected from

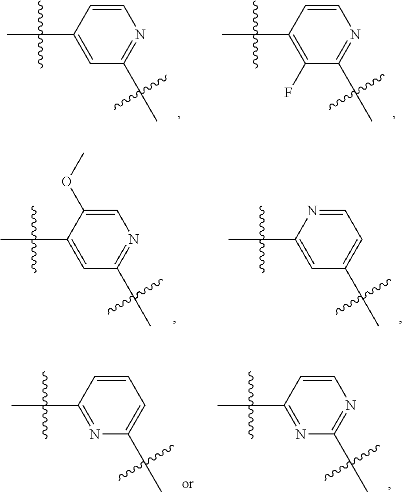

L₁ is selected from a bond,

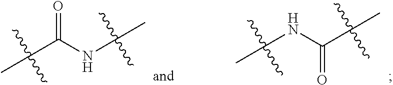 [SMILES: *NC(*)=O ; *NC(*)=O]

and the definitions of other substituents are consistent with those of embodiment seventeen, nineteen or twenty-one.

Embodiment twenty-two of the present disclosure relates to a compound of formula (I-2), an isomer thereof or a pharmaceutically acceptable salt thereof, wherein

Y₃ is selected from

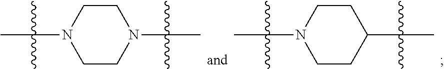 [SMILES: *N1CCN(*)CC1 ; *C1CCN(*)CC1]

and/or
Y₂ is selected from

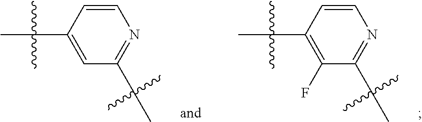 [SMILES: *c1ccnc(*)c1 ; *c1ccnc(*)c1F]

and/or
L₁ is selected from a bond;
and the definitions of other substituents are consistent with those of embodiment seventeen, nineteen or twenty-one.

For the compound of formula (I-2), the isomer thereof or the pharmaceutically acceptable salt thereof of the present disclosure,

Y₃ is selected from

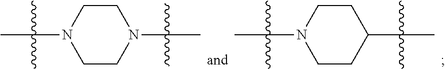 [SMILES: *N1CCN(*)CC1 ; *C1CCN(*)CC1]

Y₂ is selected from

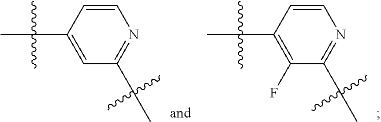 [SMILES: *c1ccnc(*)c1 ; *c1ccnc(*)c1F]

L₁ is selected from a bond;
and the definitions of other substituents are consistent with those of embodiment seventeen, nineteen or twenty-one.

Embodiment twenty-three of the present disclosure relates to a compound of formula (I-2), an isomer thereof or a pharmaceutically acceptable salt thereof, wherein

Y₁ is selected from phenyl,

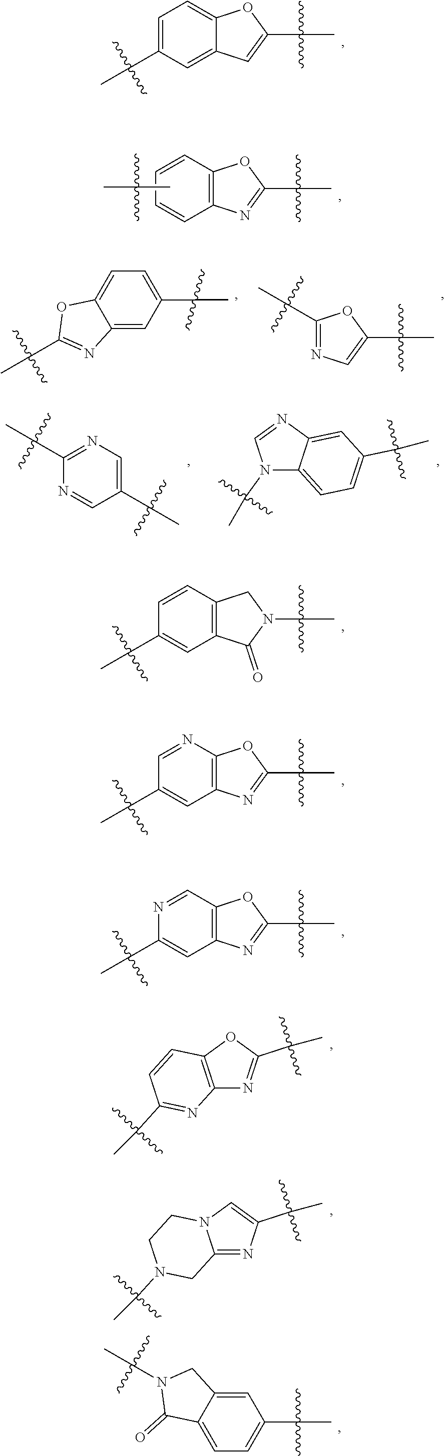

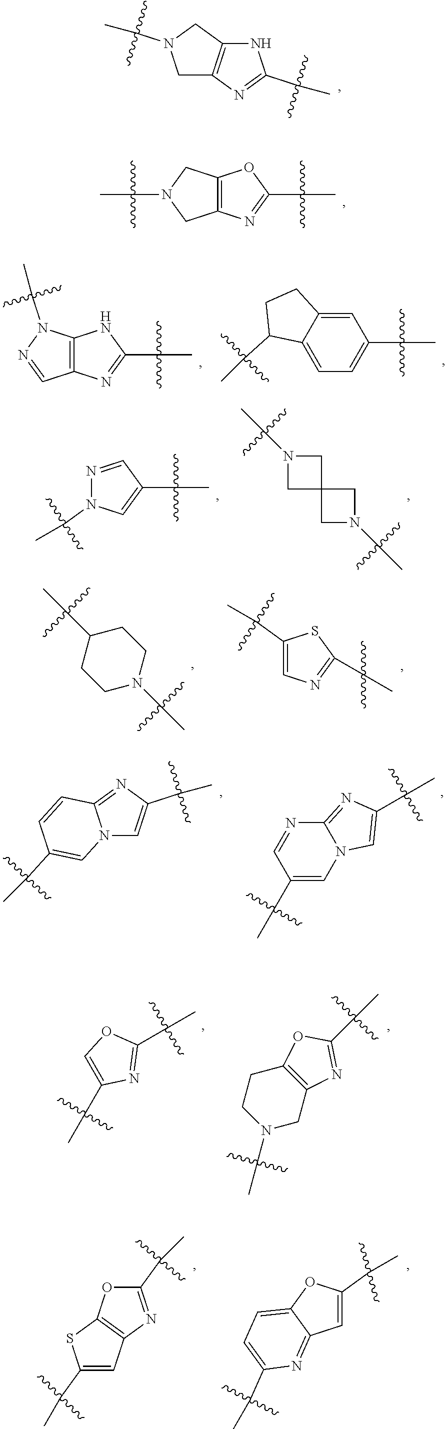

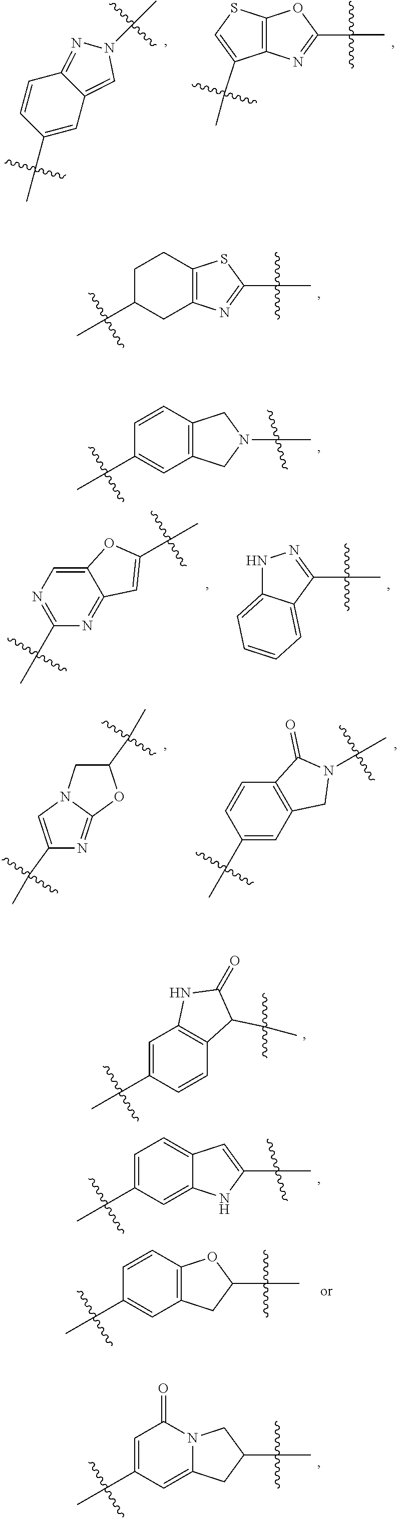

wherein the above-mentioned groups are optionally substituted with 0-3 R^{y1}; or
Y₁ and Y₂ together with L₁ form the following structure:

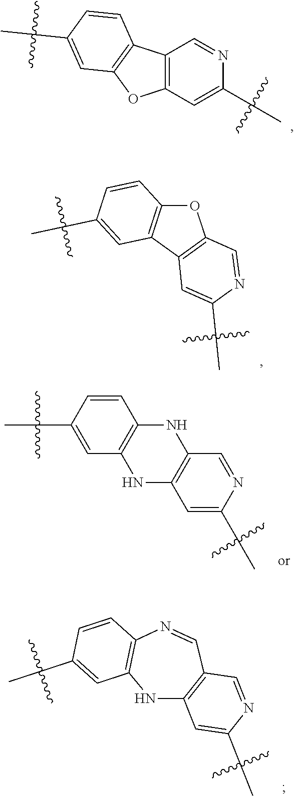 [SMILES: *c1ccc2c(c1)oc1cc(*)ncc12 ; *c1ccc2oc3cnc(*)cc3c2c1 ; *c1ccc2c(c1)Nc1cc(*)ncc1N2 ; *c1ccc2c(c1)Nc1cc(*)ncc1C=N2]

and the definitions of other substituents are consistent with those of any one of embodiments seventeen and nineteen to twenty-two.

Embodiment twenty-four of the present disclosure relates to a compound of formula (I-2), an isomer thereof or a pharmaceutically acceptable salt thereof, wherein

Y₁ is selected from

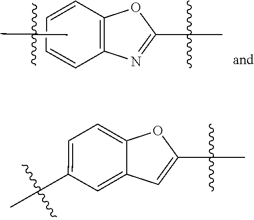

and optionally substituted with 0-3 R^{y1}; further, Y₁ is selected from

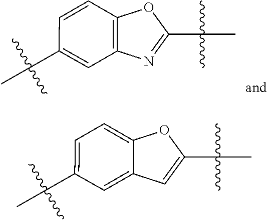 [SMILES: *c1ccc2oc(*)nc2c1 ; *c1ccc2oc(*)cc2c1]

and optionally substituted with 0-3 R^{y1};
R^{y1} is selected from F, methyl, difluoromethyl or trifluoromethyl;
and the definitions of other groups are consistent with those of any one of embodiments seventeen and nineteen to twenty-two.

Embodiment twenty-five of the present disclosure relates to a compound of formula (I-2), an isomer thereof or a pharmaceutically acceptable salt thereof, wherein

Y₁ is selected from

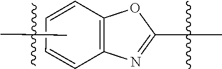

and optionally substituted with 0-3 R¹; further, Y₁ is selected from

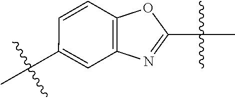 [SMILES: *c1ccc2oc(*)nc2c1]

and optionally substituted with 0-3 R^{y1};
R^{y1} is selected from F, methyl, difluoromethyl or trifluoromethyl; and the definitions of other groups are consistent with those of any one of embodiments seventeen and nineteen to twenty-four.

Embodiment twenty-six of the present disclosure relates to a compound of formula (I-2), an isomer thereof or a pharmaceutically acceptable salt thereof, wherein R₁₂ is selected from

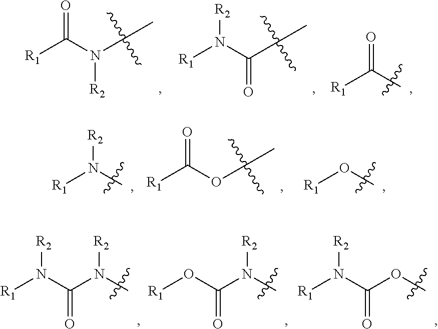

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; each R₂ is independently selected from: H or C₁₋₈ alkyl;
and the definitions of other groups are consistent with those of any one of embodiments seventeen and nineteen to twenty-five.

Embodiment twenty-seven of the present disclosure relates to a compound of formula (I-2), an isomer thereof or a pharmaceutically acceptable salt thereof, wherein

R₁₂ is selected from

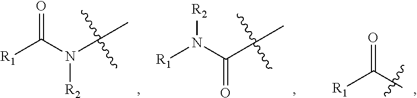

3-8 membered heterocyclyl or 5-6 membered heteroaryl, wherein the heterocyclyl and heteroaryl are optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl;
R₁ is selected from: C₁₋₄ alkyl, C₃₋₆ cycloalkyl, 3-6 membered heterocyclyl or 5-6 membered heteroaryl, wherein the alkyl, cycloalkyl, heterocyclyl or heteroaryl is optionally further substituted with 0-3 groups selected from: deuterium, hydroxyl, halogen, cyano, ═O, C₁₋₄ alkyl, C₁₋₄ alkoxy or C₁₋₄ haloalkyl;
each R₂ is independently selected from: H or C₁₋₂ alkyl;
and the definitions of other groups are consistent with those of any one of embodiments seventeen and nineteen to twenty-five.

Embodiment twenty-eight of the present disclosure relates to a compound of formula (I-2), an isomer thereof or a pharmaceutically acceptable salt thereof, wherein

R₁₂ is selected from acetyl, pyridyl, thiazolyl, pyrazinyl, acetamido,

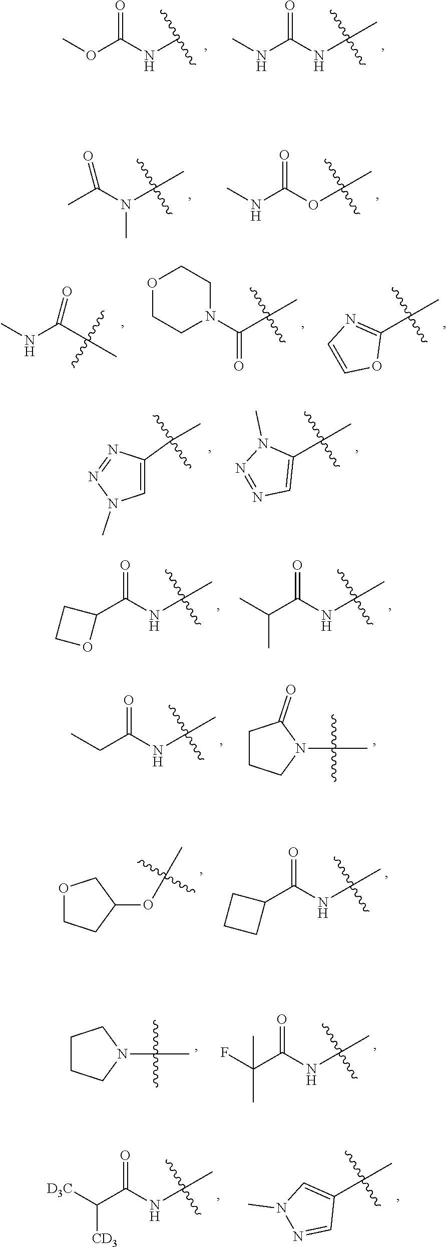

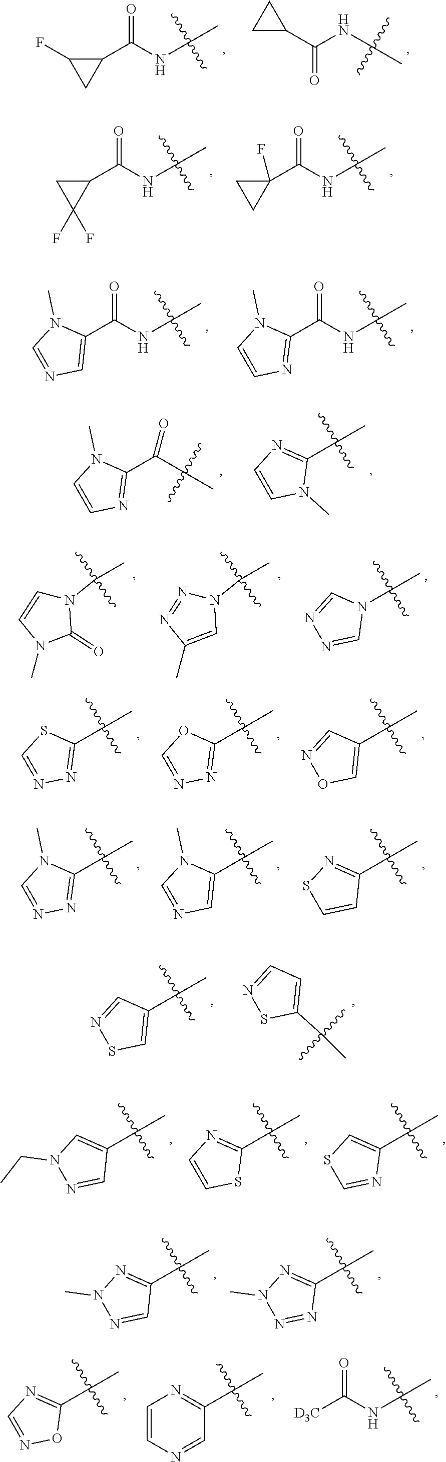

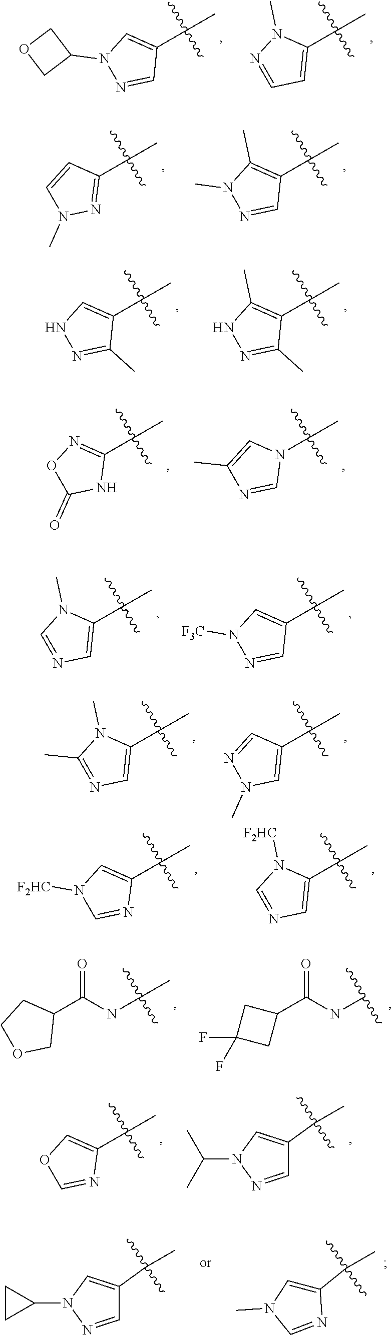

and the definitions of other groups are consistent with those of any one of embodiments seventeen and nineteen to twenty-five.

Embodiment twenty-nine of the present disclosure relates to a compound of formula (I-2), an isomer thereof or a pharmaceutically acceptable salt thereof, wherein

R₁₂ is selected from

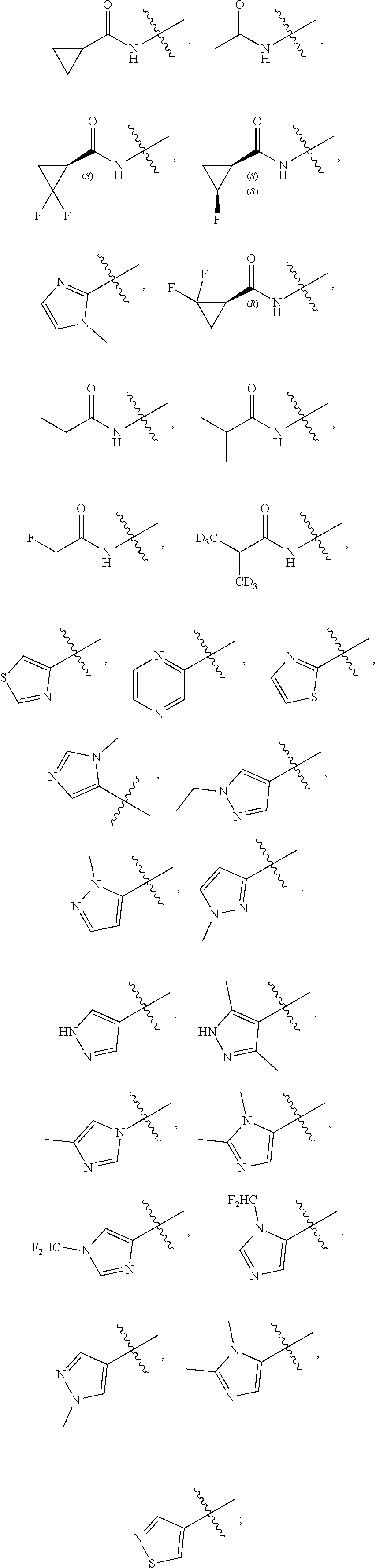

and the definitions of other groups are consistent with those of any one of embodiments seventeen and nineteen to twenty-seven.

Embodiment thirty of the present disclosure relates to a compound of formula (I-a), an isomer thereof or a pharmaceutically acceptable salt thereof, wherein R¹² is selected from 3-8 membered heterocyclyl, 5-10 membered heteroaryl or

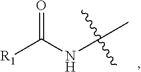

wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy, C₃₋₈ cycloalkyl, 3-8 membered heterocyclyl, or C₁₋₈ haloalkyl;
R₁ is selected from C₃₋₈ cycloalkyl, wherein the cycloalkyl is optionally further substituted with 0-3 halogen groups;
R^{y1} is selected from halogen, cyano and C₁₋₈ haloalkyl;
and the definitions of other groups are consistent with those of embodiment seventeen of the present disclosure.

Embodiment thirty-one of the present disclosure relates to a compound of formula (I-a), an isomer thereof or a pharmaceutically acceptable salt thereof, having a structure of formula (I-3) or formula (I-3-1),

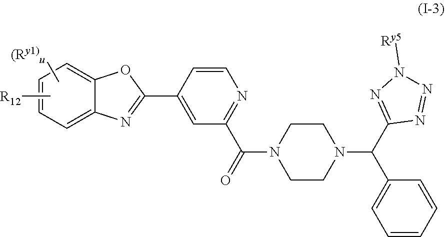

wherein,
R₁₂ is selected from 3-8 membered heterocyclyl, 5-10 membered heteroaryl and

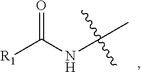

wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy, C₃₋₈ cycloalkyl or C₁₋₈ haloalkyl;
R^{y1} is selected from F, methyl, difluoromethyl or trifluoromethyl;
R^{y5} is selected from C₁₋₈ alkyl, C₁₋₈ haloalkyl, C₁₋₈ alkyl-C₁₋₈ alkoxy, C₁₋₈ hydroxylalkyl or C₃₋₈ cycloalkyl; preferably C₁₋₄ alkyl, C₁₋₄ haloalkyl, C₁₋₄ alkyl-C₁₋₄ alkoxy, C₁₋₄ hydroxylalkyl or C₃₋₆ cycloalkyl; preferably C₁₋₈ haloalkyl, C₁₋₈ alkyl-C₁₋₈ alkoxy and C₁₋₈ hydroxylalkyl; and preferably C₁₋₄ haloalkyl;
u is selected from 0, 1 or 2;
and the definitions of other groups are consistent with those of embodiment seventeen or thirty of the present disclosure.

Embodiment thirty-two of the present disclosure relates to a compound of formula (I-a), an isomer thereof or a pharmaceutically acceptable salt thereof, wherein R₁₂ is selected from 5-6 membered heteroaryl and

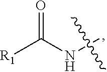

wherein the heteroaryl is optionally substituted with 0 to 3 of the following groups: halogen, ═O, acetamido, hydroxyl, C₁₋₄ alkyl, C₁₋₄ alkoxy, C₃₋₆ cycloalkyl or C₁₋₄ haloalkyl;
R₁ is selected from C₃-5 cycloalkyl, wherein the cycloalkyl is optionally further substituted with 0-2 halogen groups;
R^{y5} is selected from methyl, monofluoromethyl, difluoromethyl, monofluoroethyl, trifluoromethyl, hydroxymethyl, hydroxyethyl and methoxymethyl, and in certain embodiments, R^{y5} is selected from methyl, difluoromethyl, monofluoroethyl and trifluoromethyl;
u is selected from 0;
and the definitions of other groups are consistent with those of embodiment seventeen, thirty, or thirty-one of the present disclosure.

Embodiment thirty-three of the present disclosure relates to a compound of formula (I-a), an isomer thereof or a pharmaceutically acceptable salt thereof, wherein R₁₂ is selected from

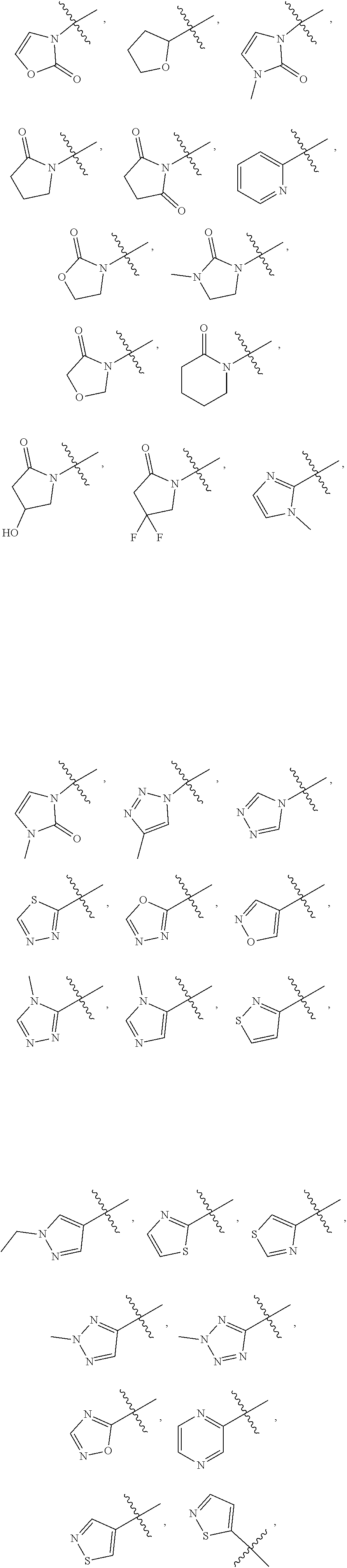

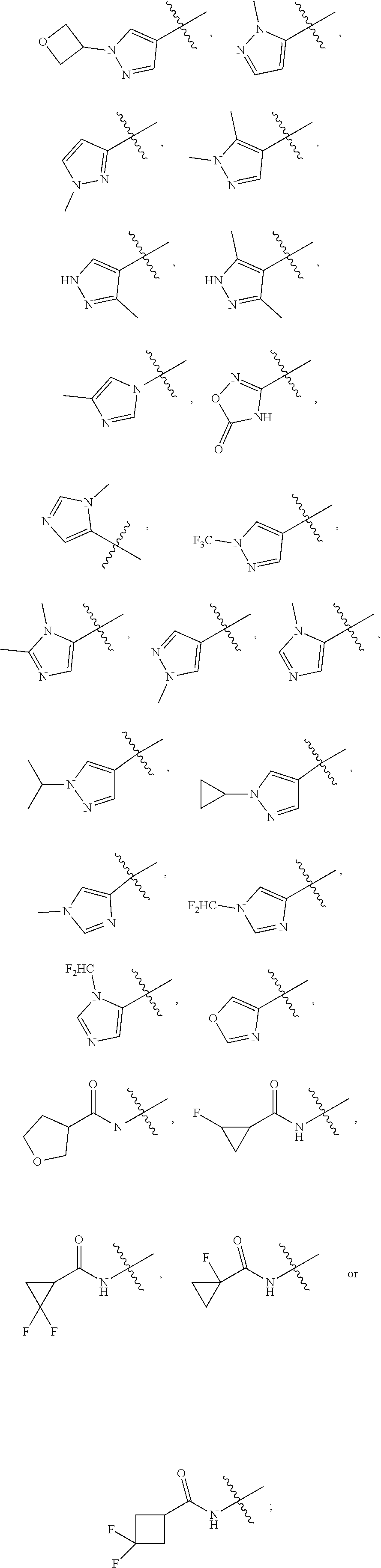

and the definitions of other groups are consistent with those of any one of embodiments seventeen and thirty to thirty-two of the present disclosure.

Embodiment thirty-four of the present disclosure relates to a compound of formula (I-a), an isomer thereof or a pharmaceutically acceptable salt thereof, having a structure of formula (I-9) or (I-9-1),

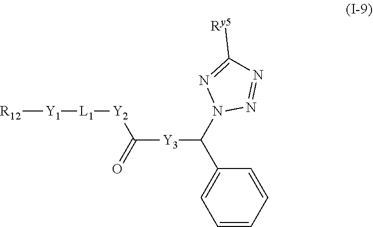

wherein each R^{y5} is independently selected from deuterium, halogen, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ hydroxylalkyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl, 5-10 membered heteroaryl and acetoxy; further, R^{y5} is selected from C₁₋₃ alkyl and C₃₋₈ cycloalkyl, wherein the alkyl is optionally further substituted with 0-5 groups selected from: deuterium, hydroxyl, acetoxy, halogen, C₁₋₃ alkoxy, amino and C₁₋₃ alkylamino; in certain embodiments, R^{y5} is selected from methyl, hydroxymethyl, acetoxymethyl, hydroxyethyl, aminomethyl, methylaminomethyl, difluoromethyl, monofluoroethyl, —CD₃ and methoxymethyl; in certain embodiments, R^{y5} is selected from methyl and difluoromethyl;
Y₄ is selected from 5-15 membered heteroaryl or phenyl, wherein the phenyl is further substituted with 1-5 R^{y4}, and the heteroaryl is optionally substituted with 1-5 R^{y4}; further, Y₄ is selected from 5 membered heteroaryl, or phenyl which is further substituted with 1-5 deuterium and halogen; in certain embodiments, Y₄ is selected from thienyl, perdeuterated phenyl, or fluorophenyl;
R¹² is selected from cyano,

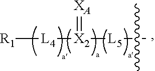

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
L₁ is selected from a bond; and the definitions of other groups are consistent with those of embodiment seventeen of the present disclosure.

Embodiment thirty-five of the present disclosure relates to a compound of formula (I-9) or (I-9-1), an isomer thereof or a pharmaceutically acceptable salt thereof, wherein

Y₃ is selected from

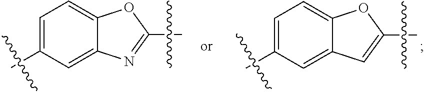

Y₂ is selected from

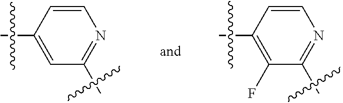

or selected from

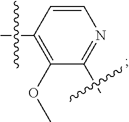

Y₃ is selected from

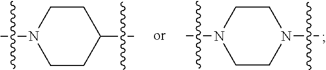

R^{y5} is selected from methyl, difluoromethyl, fluoroethyl, hydroxymethyl, hydroxyethyl, cyclopropyl, cyclobutyl, aminomethyl, —CH₂NHCH₃, —CH₂OC(O)CH₃ and —CD₃; in certain embodiments, R^{y5} is selected from methyl, difluoromethyl, fluoroethyl, hydroxymethyl, hydroxyethyl, cyclopropyl and cyclobutyl; in certain embodiments, R^{y5} is selected from methyl and difluoromethyl;
and the definitions of other groups are consistent with those of any one of embodiments seventeen and thirty-four of the present disclosure.

Embodiment thirty-six of the present disclosure relates to a compound of formula (I-9) or (I-9-1), an isomer thereof or a pharmaceutically acceptable salt thereof, wherein

Y₁ is selected from

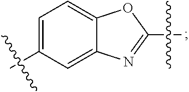

Y₂ is selected from

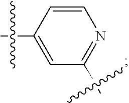

Y₃ is selected from

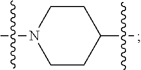

R^{y5} is selected from methyl, difluoromethyl, fluoroethyl, hydroxymethyl, hydroxyethyl, cyclopropyl, cyclobutyl, aminomethyl, —CH₂NHCH₃, —CH₂OC(O)CH₃ and —CD₃, and further, R^{y5} is selected from methyl, difluoromethyl and hydroxymethyl;
and the definitions of other groups are consistent with those of any one of embodiments seventeen and thirty-four of the present disclosure.

Embodiment thirty-seven of the present disclosure relates to a compound of formula (I-9) or (I-9-1), an isomer thereof or a pharmaceutically acceptable salt thereof, wherein

R₁₂ is selected from

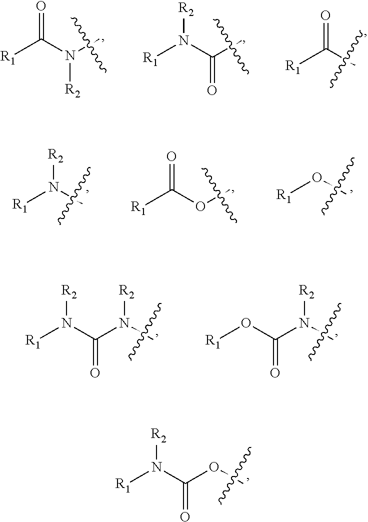

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
each R₂ is independently selected from: H or C₁₋₈ alkyl;
and the definitions of other groups are consistent with those of any one of embodiments seventeen and thirty-four to thirty-six of the present disclosure.

Embodiment thirty-eight of the present disclosure relates to a compound of formula (I-9) or (I-9-1), an isomer thereof or a pharmaceutically acceptable salt thereof, wherein

R₁₂ is selected from

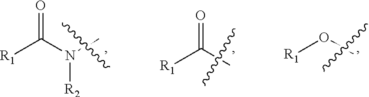

5 membered heteroaryl and 6 membered heteroaryl, wherein the heteroaryl is optionally substituted with 0-3 of the following groups: halogen, C₁₋₄ alkyl, C₁₋₄ alkoxy and C₁₋₄ haloalkyl;
R₁ is selected from three membered cycloalkyl, four membered cycloalkyl, five membered cycloalkyl and 5 membered heterocycloalkyl, wherein the cycloalkyl and heterocycloalkyl are optionally further substituted with 0-2 groups selected from: deuterium, hydroxyl, halogen, cyano, ═O, C₁-2 alkyl, C₁₋₂ alkoxy or C₁₋₂ haloalkyl; each R₂ is independently selected from: H or C₁₋₂ alkyl;
and the definitions of other groups are consistent with those of any one of embodiments seventeen and thirty-four to thirty-six of the present disclosure.

Embodiment thirty-nine of the present disclosure relates to a compound of formula (I-9) or (I-9-1), an isomer thereof or a pharmaceutically acceptable salt thereof, wherein

Y₁ is selected from

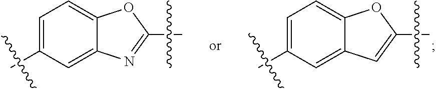

Y₂ is selected from

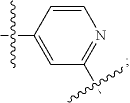

Y₃ is selected from

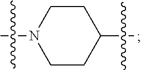

L₁ is selected from a bond;
R^{y5} is selected from methyl, difluoromethyl, fluoroethyl, hydroxymethyl, hydroxyethyl, cyclopropyl, cyclobutyl, aminomethyl, —CH₂NHCH₃, —CH₂OC(O)CH₃ and —CD₃, and further, R^{y5} is selected from methyl, difluoromethyl and hydroxymethyl;
R₁₂ is selected from

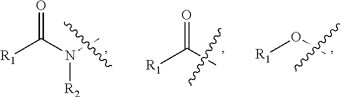

5 membered heteroaryl and 6 membered heteroaryl, wherein the heteroaryl is optionally substituted with 0-3 of the following groups: halogen, C₁₋₄ alkyl, C₁₋₄ alkoxy and C₁₋₄ haloalkyl;
R₁ is selected from 3 membered cycloalkyl, 4 membered cycloalkyl, 5 membered cycloalkyl, 5 membered heterocycloalkyl and 5 membered heteroaryl, wherein the cycloalkyl and heterocycloalkyl are optionally further substituted with 0-2 groups selected from: deuterium, hydroxyl, halogen, cyano, ═O, C₁₋₂ alkyl, C₁₋₂ alkoxy or C₁₋₂ haloalkyl;
and each R₂ is independently selected from: H or C₁₋₂ alkyl.

Embodiment forty of the present disclosure relates to a compound of formula (I-9) or (I-9-1), an isomer thereof or a pharmaceutically acceptable salt thereof, wherein

R₁₂ is selected from acetyl, pyridyl, thiazolyl, pyrazinyl, acetamido,

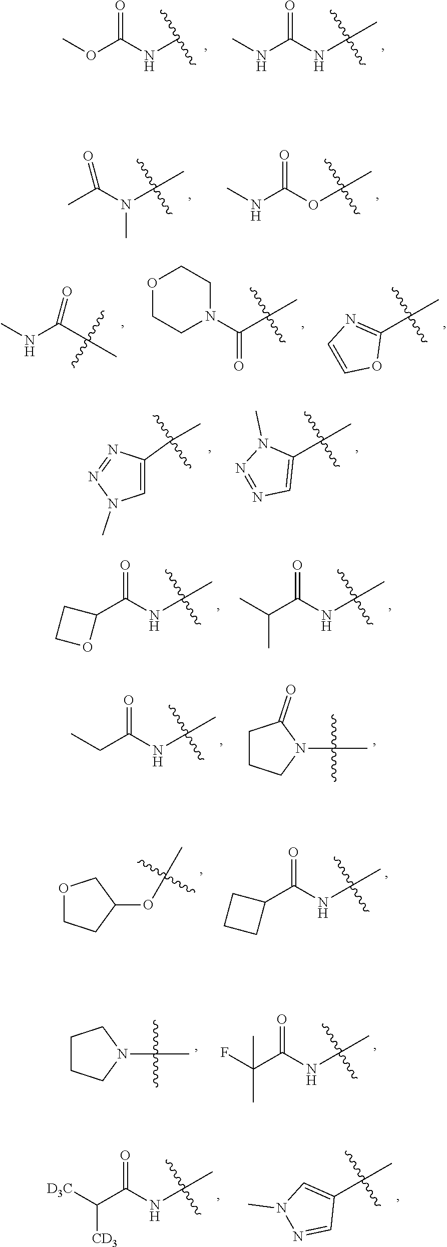

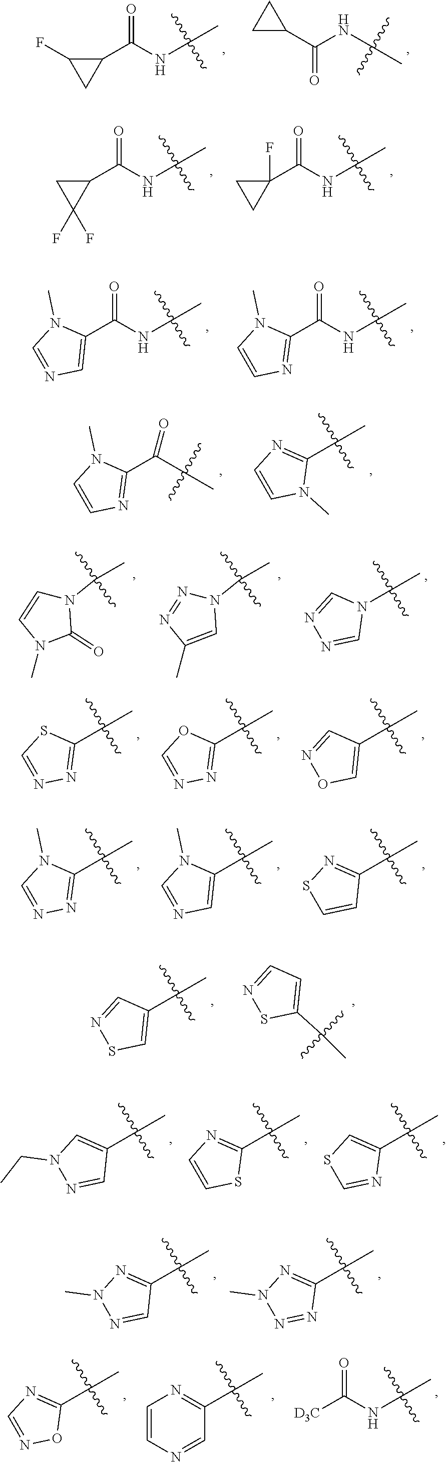

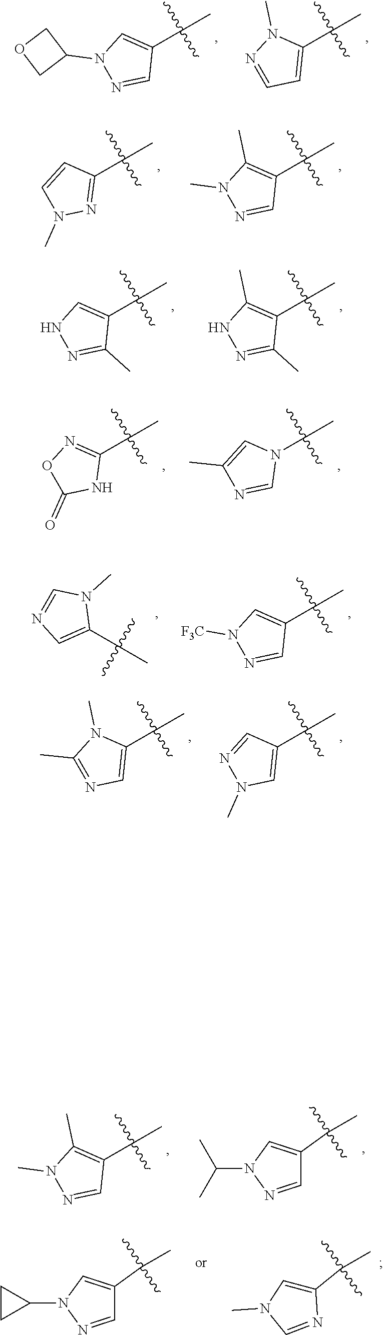

and the definitions of other groups are consistent with those of any one of embodiments seventeen and thirty-four to thirty-six of the present disclosure.

In embodiment forty-one of the present disclosure, for the compound, the isomer thereof or the pharmaceutically acceptable salt thereof of any one of embodiments seventeen to twenty-seven, embodiments thirty to thirty-two and embodiments thirty-four to thirty-nine, R₁₂ is selected from

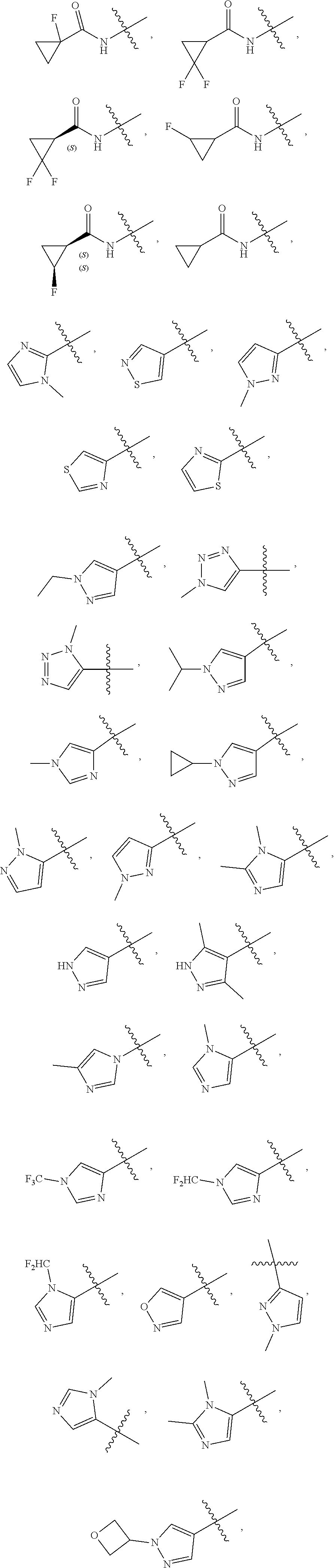

further, R₁₂ is selected from

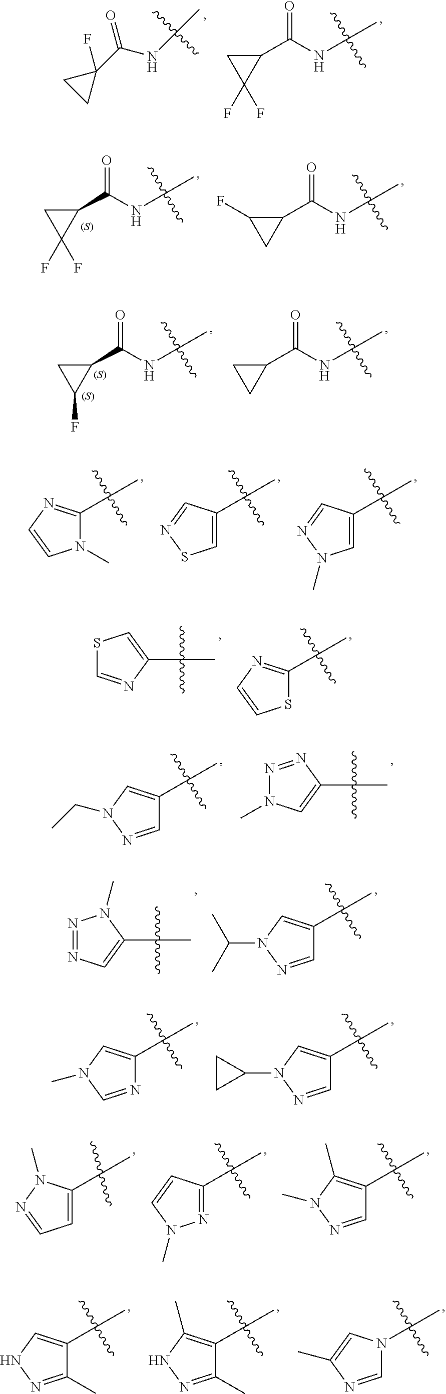

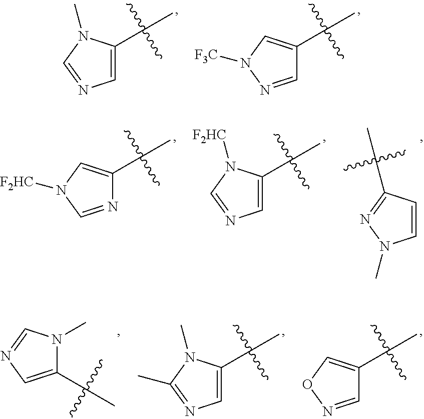

and further, R₁₂ is selected from

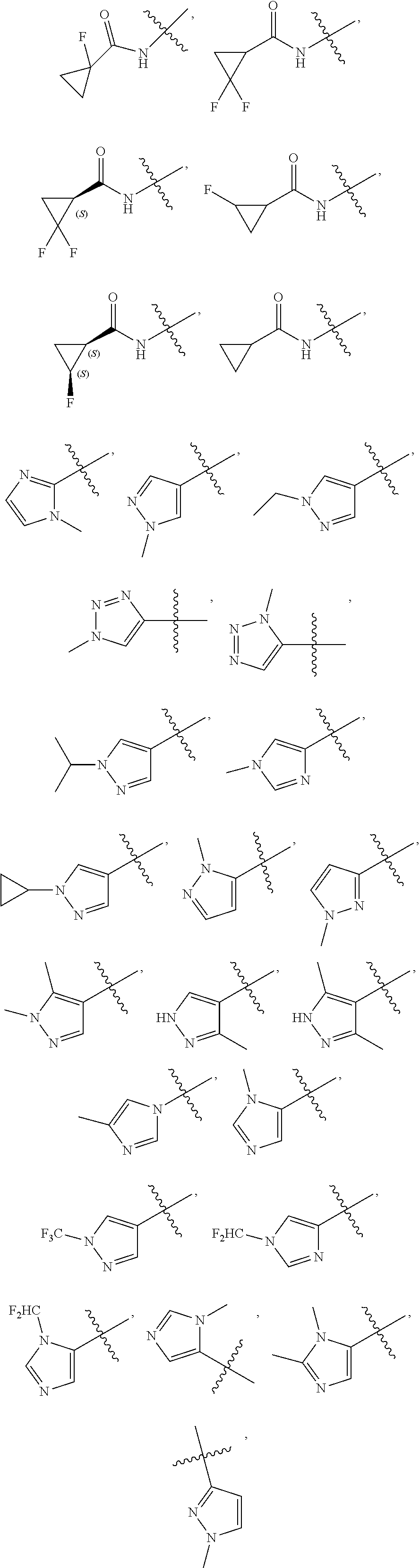

In any one of the above-mentioned technical embodiments herein, Y₃ is selected from

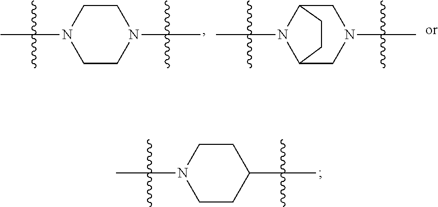 [SMILES: *N1CCN(*)CC1 ; *N1CC2CCC(C1)N2* ; *C1CCN(*)CC1]

further, Y₃ is selected from

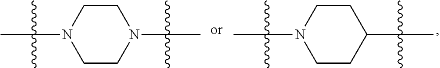 [SMILES: *N1CCN(*)CC1 ; *C1CCN(*)CC1]

further, Y₃ is selected from

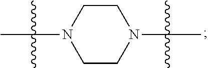 [SMILES: *N1CCN(*)CC1]

and further, Y₃ is selected from

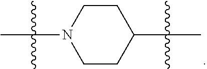 [SMILES: *C1CCN(*)CC1]

In any one of the above-mentioned technical embodiments herein, Y₂ is selected from

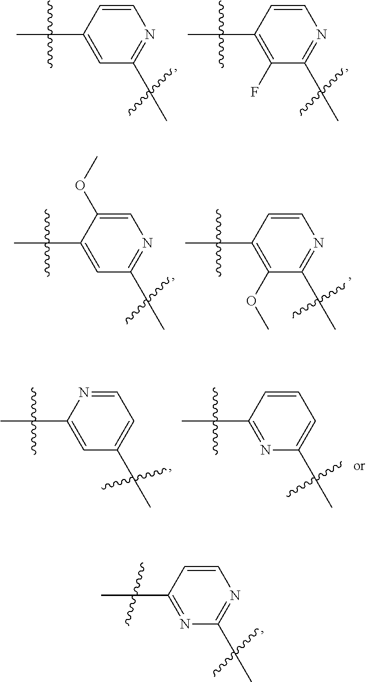

further, Y₂ is selected from

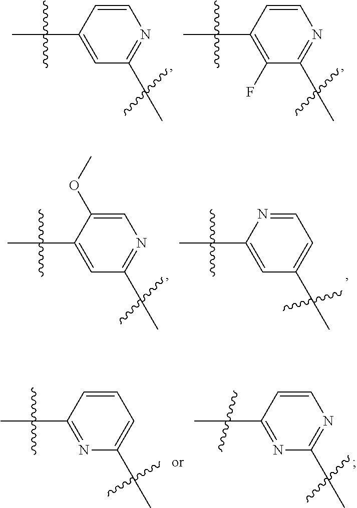

further, Y₂ is selected from

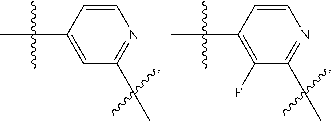 [SMILES: *c1ccnc(*)c1 ; *c1ccnc(*)c1F]

or is selected from

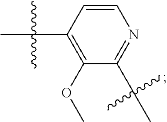 [SMILES: *c1ccnc(*)c1OC]

further, Y₂ is selected from

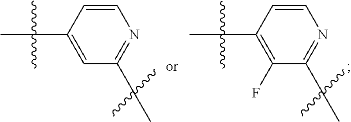 [SMILES: *c1ccnc(*)c1 ; *c1ccnc(*)c1F]

further, Y₂ is selected from

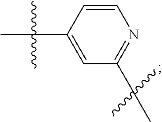 [SMILES: *c1ccnc(*)c1]

and further, Y₂ is selected from

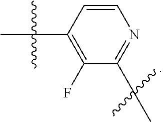 [SMILES: *c1ccnc(*)c1F]

In any one of the above-mentioned technical embodiments herein, L₁ is selected from a bond,

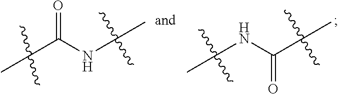 [SMILES: *NC(*)=O ; *NC(*)=O]

and further, L₁ is selected from a bond.

In any one of the above-mentioned technical embodiments herein, Y₁ is selected from phenyl,

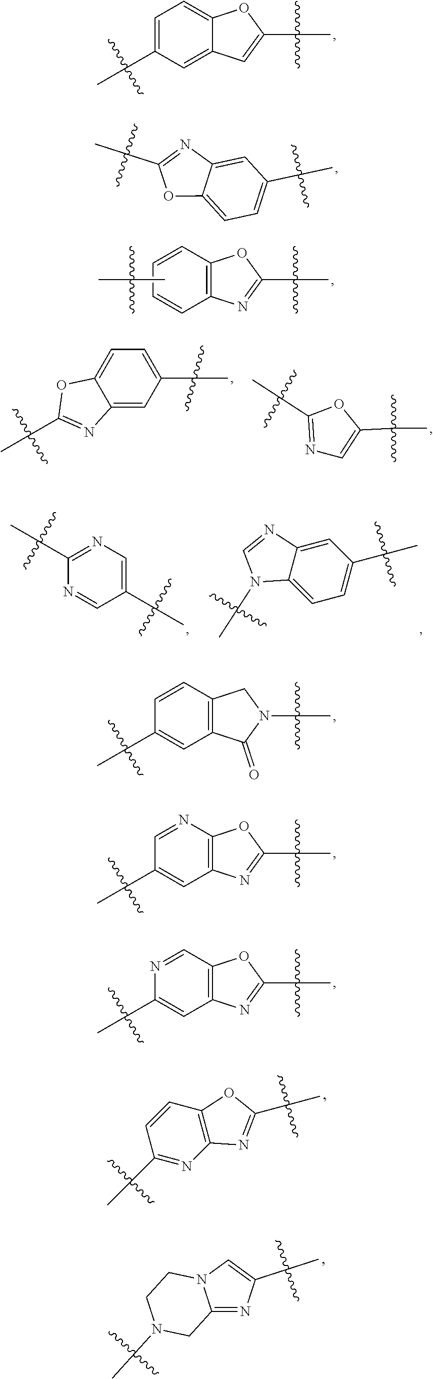

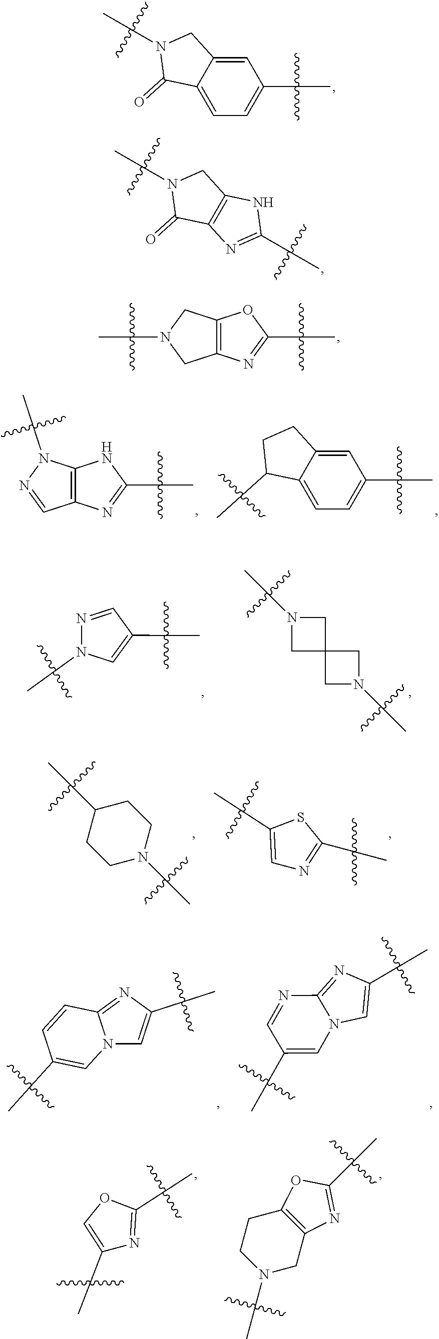

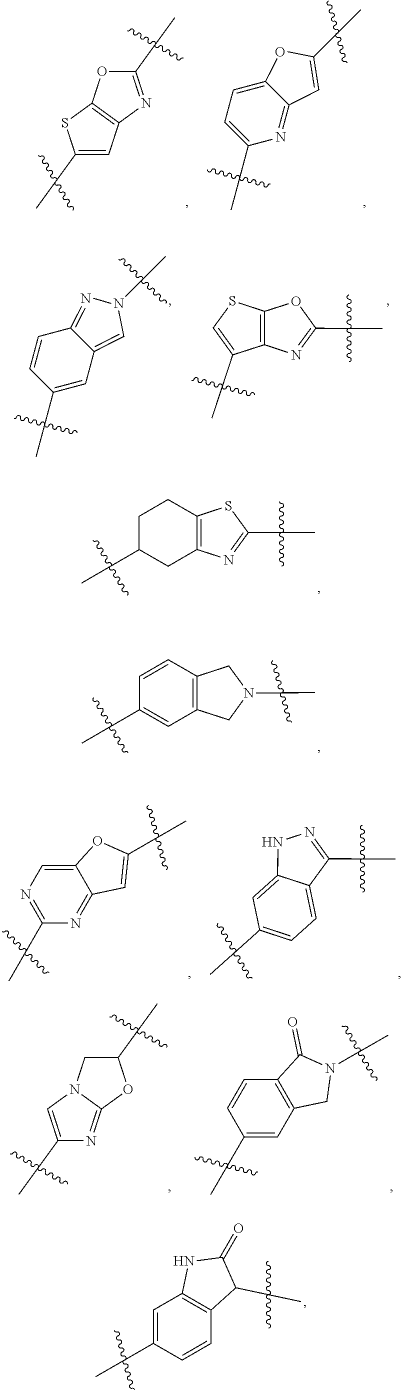

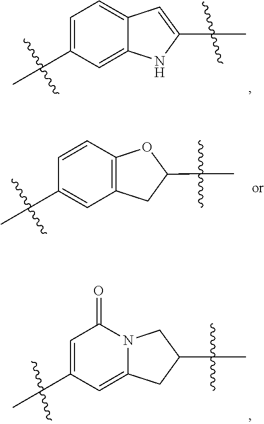 [SMILES: *c1ccc2cc(*)[nH]c2c1 ; *c1ccc2c(c1)CC(*)O2 ; *c1cc2n(c(=O)c1)CC(*)C2]

wherein the above-mentioned groups are optionally substituted with 0-3 R^{y1}; further Y₁ is selected from

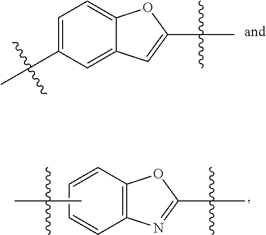

wherein the above-mentioned groups are optionally substituted with 0-3 R^{y1}; further Y₁ is selected from

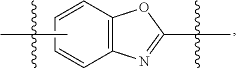

optionally substituted with 0-3 R^{y1}; further Y₁ is selected from

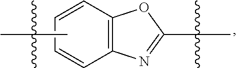

wherein the above-mentioned groups are optionally substituted with 0-3 R^{y1}; further, Y₁ is selected from

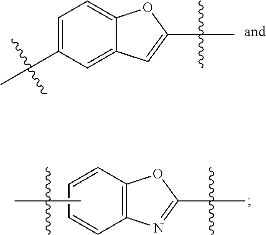

further Y₁ is selected from

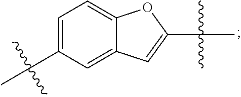 [SMILES: *c1ccc2oc(*)cc2c1]

and further, Y₁ is selected from

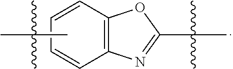

In any one of the above-mentioned technical embodiments herein, R^{y1} is selected from halogen, cyano, C₁₋₈ alkyl and C₁₋₈ haloalkyl; further, R^{y1} is selected from F, methyl, difluoromethyl or trifluoromethyl; and further, R^{y1} is selected from F and methyl.

In any one of the above-mentioned technical embodiments herein, Y₄ is selected from phenyl optionally substituted with 0-5 R^{y4}; further, Y₄ is selected from phenyl and thienyl optionally substituted with 0-5 R^{y4}; further, Y₄ is selected from phenyl optionally substituted with 0-5 R^{y4}; and further, Y₄ is selected from phenyl.

In any one of the above-mentioned technical embodiments herein, Y₅ is selected from tetrazolyl, wherein the tetrazolyl is further substituted with difluoromethyl, fluoroethyl and hydroxymethyl; further, Y₅ is selected from

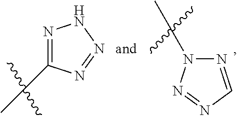 [SMILES: *c1nn[nH]n1 ; *n1ncnn1]

wherein the above-mentioned groups are further substituted with monofluoromethyl, difluoromethyl, trifluoromethyl, fluoroethyl, hydroxyethyl, hydroxymethyl and methoxymethyl; further, Y₅ is selected from

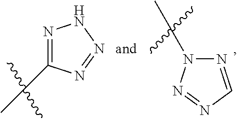 [SMILES: *c1nn[nH]n1 ; *n1ncnn1]

wherein the above-mentioned groups are further substituted with monofluoromethyl, difluoromethyl, trifluoromethyl, fluoroethyl, hydroxyethyl, hydroxymethyl and methoxymethyl; further, Y₅ is selected from

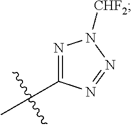 [SMILES: *c1nnn(C(F)F)n1]

further, Y₅ is selected from

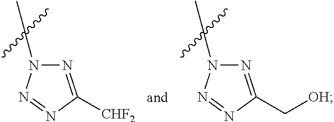 [SMILES: *n1nnc(C(F)F)n1 ; *n1nnc(CO)n1]

land further, Y₅ is selected from

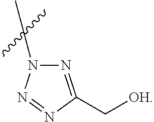 [SMILES: *n1nnc(CO)n1]

In any one of the above-mentioned technical embodiments herein, Y₅ is selected from

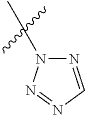 [SMILES: *n1ncnn1]

optionally further substituted with 1 R^{y5}, wherein R^{y5} is selected from deuterium, halogen, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ hydroxylalkyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl, 5-10 membered heteroaryl and acetoxy; further, R^{y5} is selected from deuterium, halogen, hydroxyl, cyano, nitro, C₁₋₄ alkyl, C₁₋₄ alkoxy, C₂₋₄ alkenyl, C₂₋₄ alkynyl, C₃₋₆ cycloalkyl, 3-6 membered heterocyclyl, phenyl or 5-6 membered heteroaryl, wherein the alkyl, alkoxy, alkenyl, alkynyl, cycloalkyl, heterocyclyl, phenyl and heteroaryl are optionally further substituted with 1-5 groups selected from: deuterium, halogen, ═O, acetamido, hydroxyl, sulfhydryl, cyano, nitro, silyl, C₁₋₂ hydroxylalkyl, C₁₋₃ alkyl, C₁₋₃ alkoxy, C₁₋₃ haloalkyl, C₂₋₄ alkenyl, C₂₋₄ alkynyl, C₃₋₆ cycloalkyl, 3-6 membered heterocyclyl, phenyl, 5-6 membered heteroaryl and acetoxy; further, R^{y5} is selected from C₁₋₄ alkyl, C₁₋₄ alkoxy, C₂₋₄ alkenyl, C₂₋₄ alkynyl, C₃₋₆ cycloalkyl, 3-6 membered heterocyclyl, phenyl or 5-6 membered heteroaryl, wherein the alkyl, alkoxy, alkenyl, alkynyl, cycloalkyl, heterocyclyl, phenyl and heteroaryl are optionally further substituted with 1-5 groups selected from: deuterium, halogen, ═O, acetamido, hydroxyl, sulfhydryl, cyano, nitro, C₁₋₂ hydroxylalkyl, C₁₋₃ alkyl, C₁₋₃ alkoxy, C₁₋₃ haloalkyl, C₂₋₄ alkenyl, C₂-4 alkynyl, C₃₋₆ cycloalkyl, 3-6 membered heterocyclyl, phenyl, 5-6 membered heteroaryl and acetoxy.

In any one of the above-mentioned technical embodiments herein, Y₅ is selected from

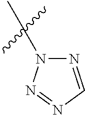 [SMILES: *n1ncnn1]

optionally further substituted with 1 R^{y5}, wherein R^{y5} is selected from C₁₋₃ alkyl and C₃₋₈ cycloalkyl, wherein the alkyl is optionally further substituted with 0-5 groups selected from: deuterium, hydroxyl, acetoxy, halogen, C₁₋₃ alkoxy, amino and C₁₋₃ alkylamino; further, Y₅ is selected from

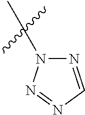 [SMILES: *n1ncnn1]

optionally further substituted with 1 R^{y5}, wherein R^{y5} is selected from C₁₋₃ alkyl, wherein the alkyl is optionally further substituted with 0-5 groups selected from: deuterium, hydroxyl, acetoxy, halogen, C₁₋₃ alkoxy, amino and C₁₋₃ alkylamino; further, Y₅ is selected from

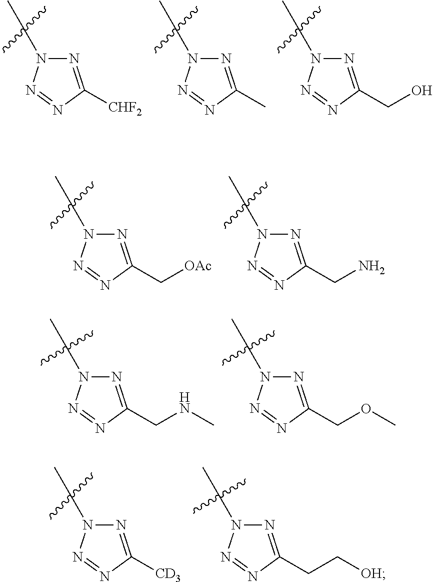

further Y₅ is selected from

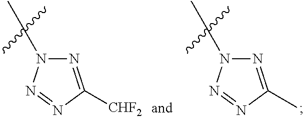

and further Y₅ is selected from

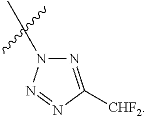 [SMILES: *n1nnc(C(F)F)n1]

In any one of the above-mentioned technical embodiments herein, Y₅ is selected from

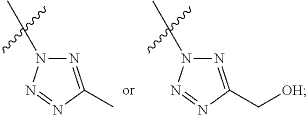

and further, Y₅ is selected from

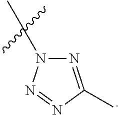 [SMILES: *n1nnc(C)n1]

In any one of the above-mentioned technical embodiments herein, L₂ is selected from carbonyl, and L₃ is selected from

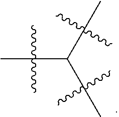 [SMILES: *C(*)*]

In any one of the above-mentioned technical embodiments herein, R₁₂ is selected from cyano,

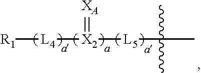

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
further, R₁₂ is selected from

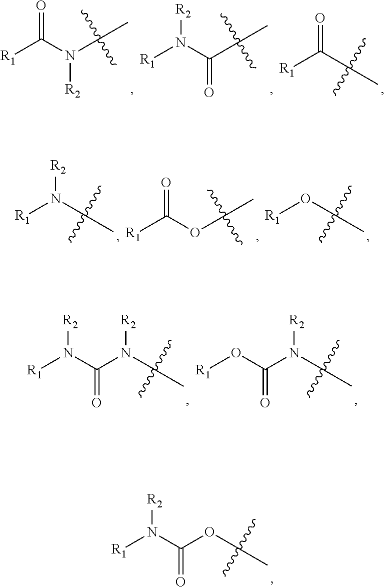

3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl; wherein R₁ is selected from C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl, and each R₂ is independently selected from: H or C₁₋₈ alkyl; further, R₁₂ is selected from

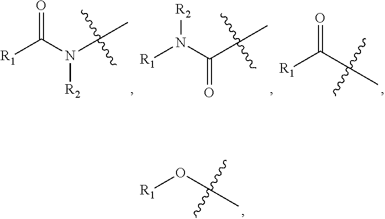

3-8 membered heterocyclyl or 5-6 membered heteroaryl, wherein the heterocyclyl and heteroaryl are optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl; wherein, R₁ is selected from C₁₋₄ alkyl, C₃₋₆ cycloalkyl, 3-6 membered heterocyclyl or 5-6 membered heteroaryl, wherein the alkyl, cycloalkyl, heterocyclyl or heteroaryl is optionally further substituted with 0-3 groups selected from: deuterium, hydroxyl, halogen, cyano, ═O, C₁₋₄ alkyl, C₁₋₄ alkoxy or C₁₋₄ haloalkyl, and each R₂ is independently selected from: H or C₁₋₂ alkyl;
further, R₁₂ is selected from

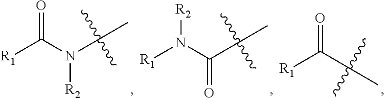

3-8 membered heterocyclyl or 5-6 membered heteroaryl, wherein the heterocyclyl and heteroaryl are optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl; wherein, R₁ is selected from C₁₋₄ alkyl, C₃₋₆ cycloalkyl, 3-6 membered heterocyclyl or 5-6 membered heteroaryl, wherein the alkyl, cycloalkyl, heterocyclyl or heteroaryl is optionally further substituted with 0-3 groups selected from: deuterium, hydroxyl, halogen, cyano, ═O, C₁₋₄ alkyl, C₁₋₄ alkoxy or C₁₋₄ haloalkyl, and each R₂ is independently selected from: H or C₁-2 alkyl;
further, R₁₂ is selected from

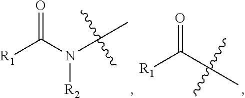

3-8 membered heterocyclyl or 5-6 membered heteroaryl, wherein the heterocyclyl and heteroaryl are optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl; wherein, R₁ is selected from C₁₋₄ alkyl, C₃₋₆ cycloalkyl, 3-6 membered heterocyclyl or 5-6 membered heteroaryl, wherein the alkyl, cycloalkyl, heterocyclyl or heteroaryl is optionally further substituted with 0-3 groups selected from: deuterium, hydroxyl, halogen, cyano, ═O, C₁₋₄ alkyl, C₁₋₄ alkoxy or C₁₋₄ haloalkyl, and each R₂ is independently selected from: H or C₁₋₂ alkyl;
further, R₁₂ is selected from

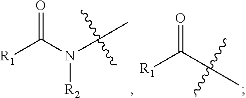

wherein, R₁ is selected from C₁₋₄ alkyl, C₃₋₆ cycloalkyl, 3-6 membered heterocyclyl or 5-6 membered heteroaryl, wherein the alkyl, cycloalkyl, heterocyclyl or heteroaryl is optionally further substituted with 0-3 groups selected from: deuterium, hydroxyl, halogen, cyano, ═O, C₁₋₄ alkyl, C₁₋₄ alkoxy or C₁₋₄ haloalkyl, and each R₂ is independently selected from: H or C₁-2 alkyl; further, R₁₂ is selected from 3-8 membered heterocyclyl; further, R₁₂ is selected from 3-6 membered heterocyclyl; further, R₁₂ is selected from 3-6 membered heterocyclyl; further, R₁₂ is selected from 5-6 membered heterocycloalkyl; and the heterocyclyl or heterocycloalkyl is optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl;
further, R₁₂ is selected from 5 membered heteroaryl and 6 membered heteroaryl, wherein the heteroaryl is optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl;
further, R₁₂ is selected from 5 membered heteroaryl, wherein the heteroaryl is optionally substituted with 0-3 of the following groups: halogen, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl;
further, R₁₂ is selected from 5 membered heteroaryl, wherein the heteroaryl is optionally substituted with 1-2 of the following groups: halogen, C₁₋₄ alkyl, C₁₋₄ alkoxy, C₁₋₄ haloalkyl, C₃₋₆ cycloalkyl or 3-6 membered heterocycloalkyl;
further, R₁₂ is selected from acetyl, pyridyl, thiazolyl, pyrazinyl, acetamido,

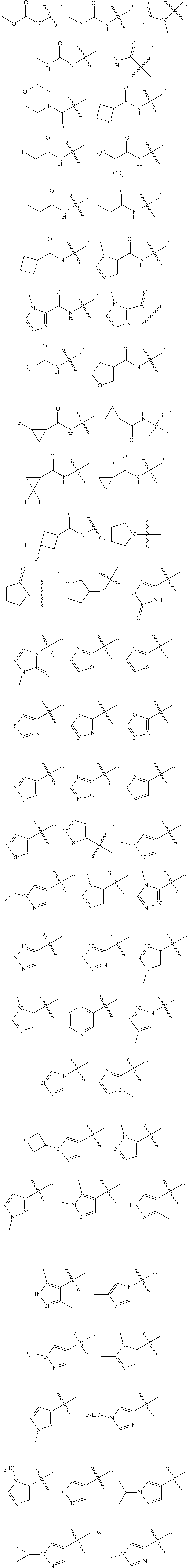

further, R₁₂ is selected from

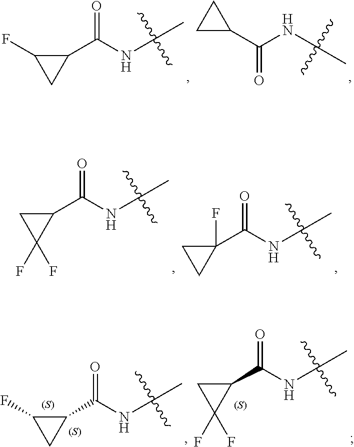

further, R₁₂ is selected from:

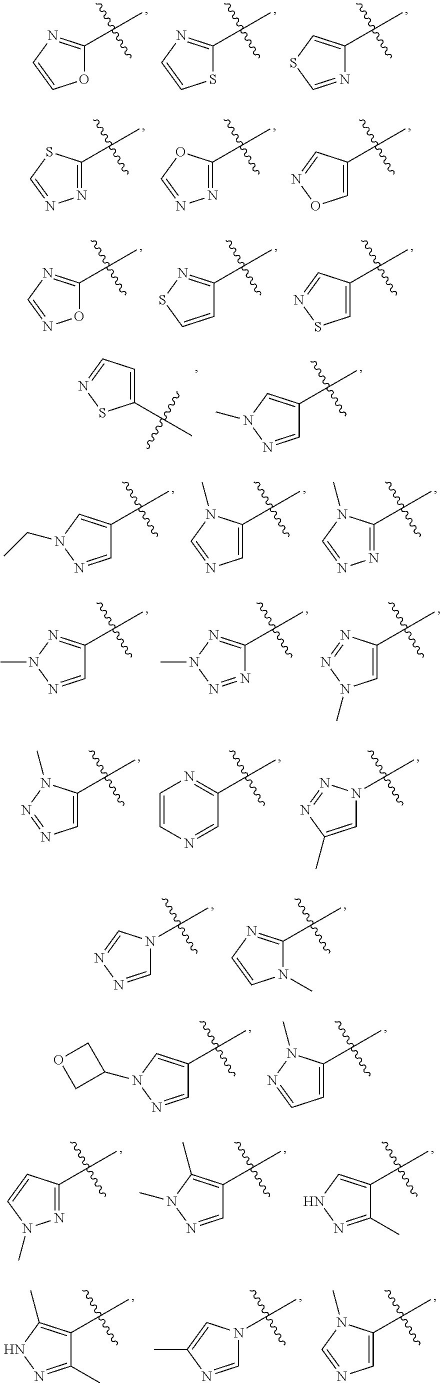

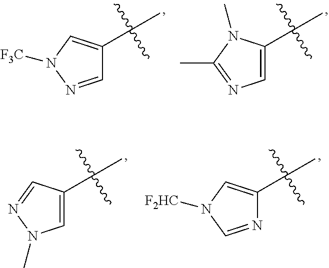

further, R₁₂ is selected from:

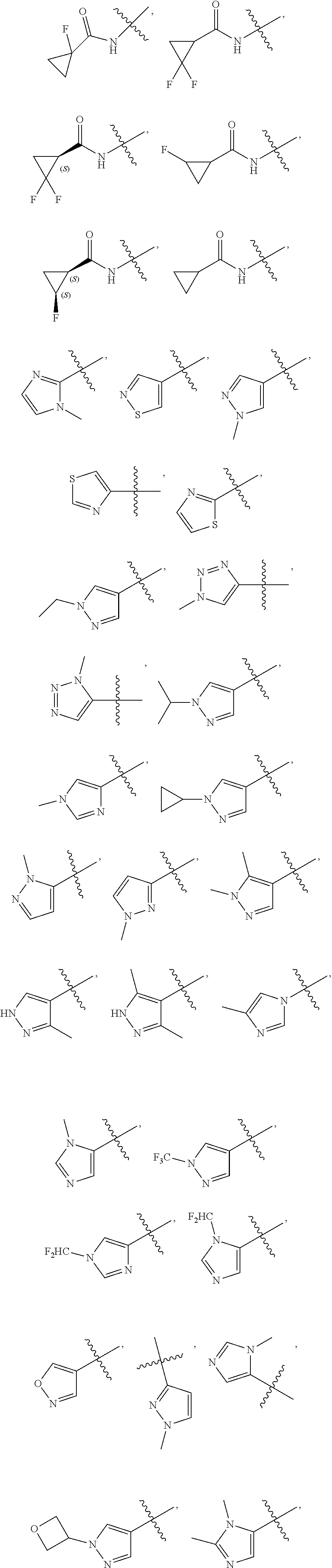

further, R₁₂ is selected from acetamido,

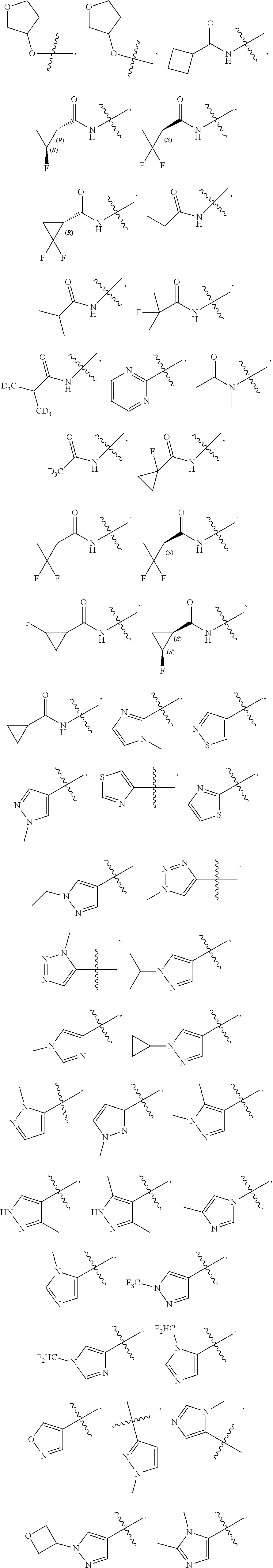

The above-mentioned compound of formula (I-a) of the present disclosure has the following configurations:

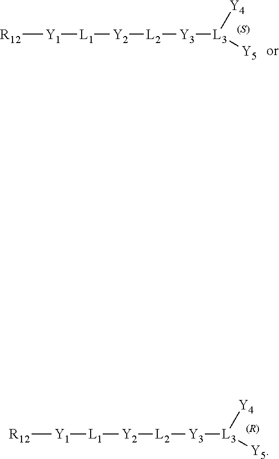

The above-mentioned compound of formula (I-2) of the present disclosure has the following configurations:

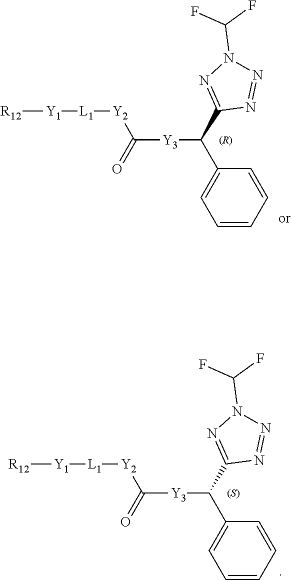

The above-mentioned compound of formula (I-3) of the present disclosure has the following configurations:

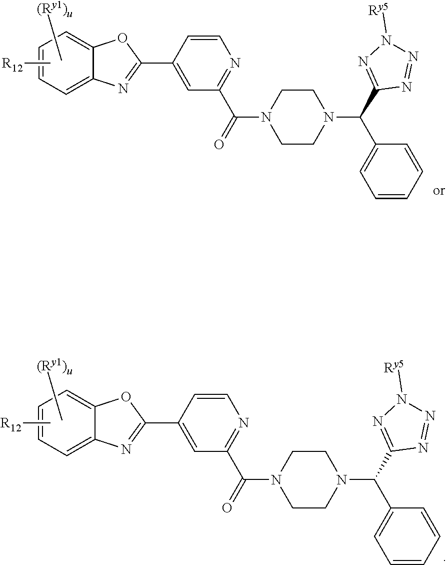

The above-mentioned compound of formula (I-3-1) of the present disclosure has the following configurations:

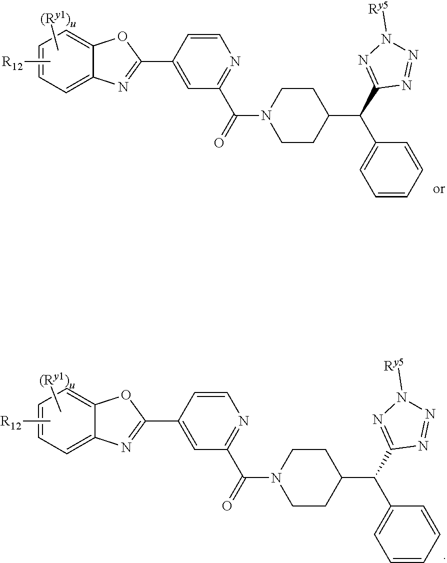

The above-mentioned compound of formula (I-9) of the present disclosure has the following configurations:

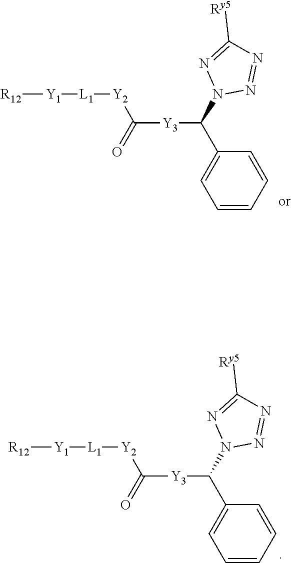

In the compound of the present disclosure [symbol] represents a single bond or a double bond; and in certain embodiments, [symbol] represents a single bond.

[symbol] of the present disclosure represents a group linking site, and when there are more than one group linking sites, groups can be linked arbitrarily, which can mean that the left side is linked to the left group, and the right side is linked to the right group when viewed from the front.

The present disclosure discloses a compound as shown below, an isomer thereof or a pharmaceutically acceptable salt thereof:

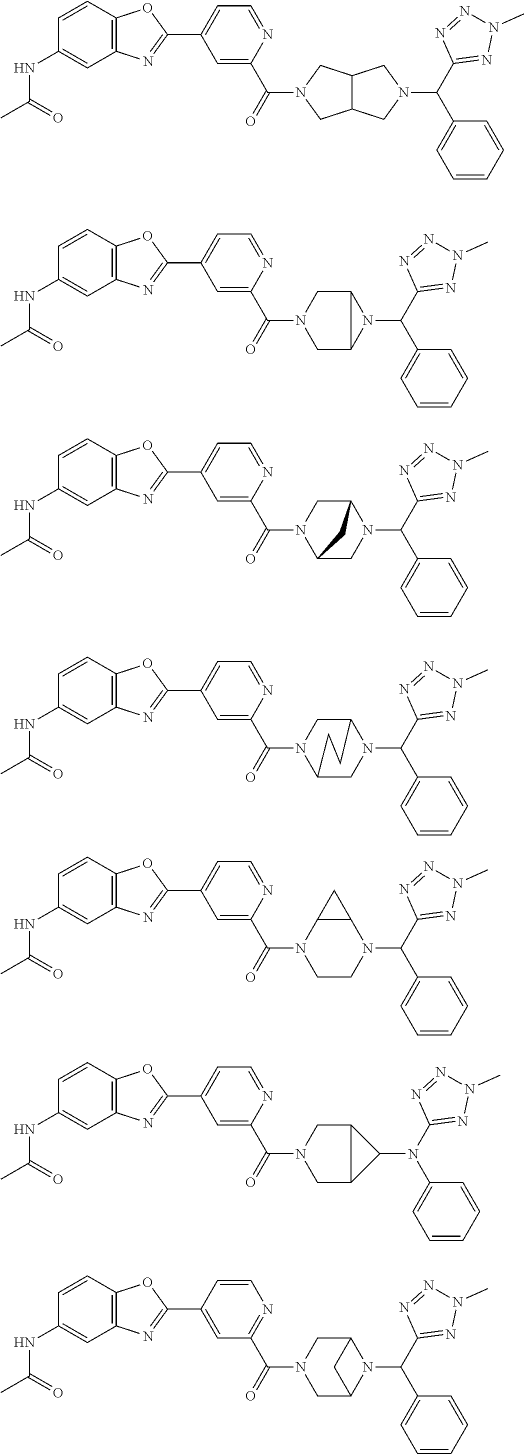

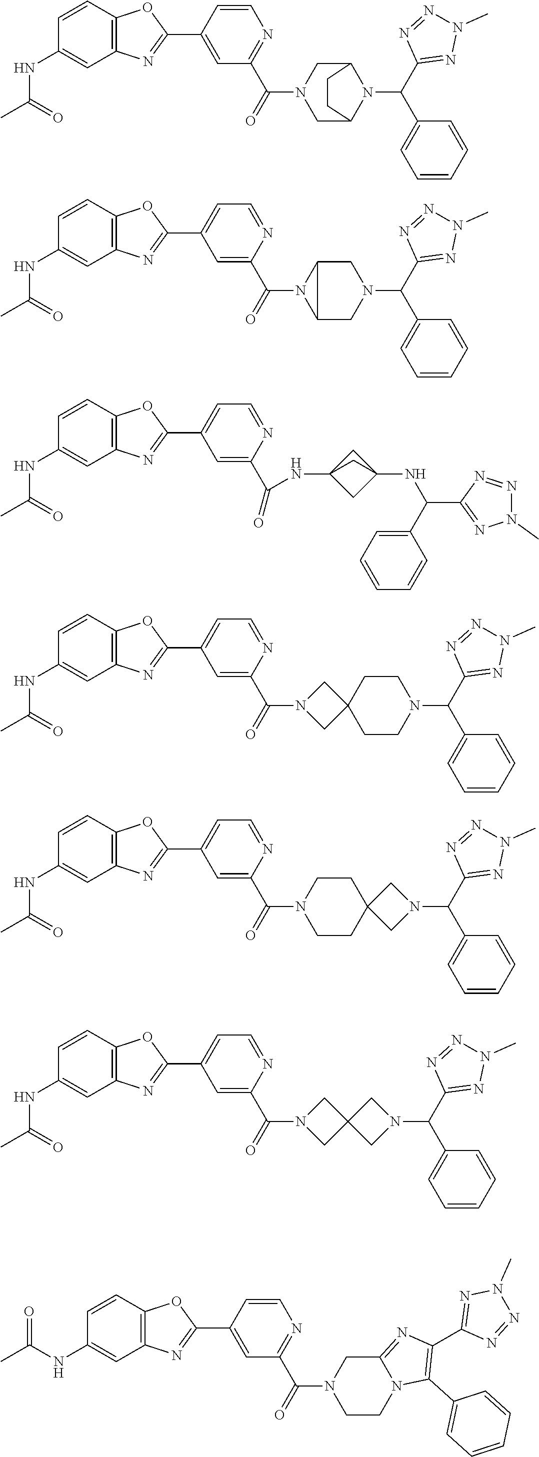

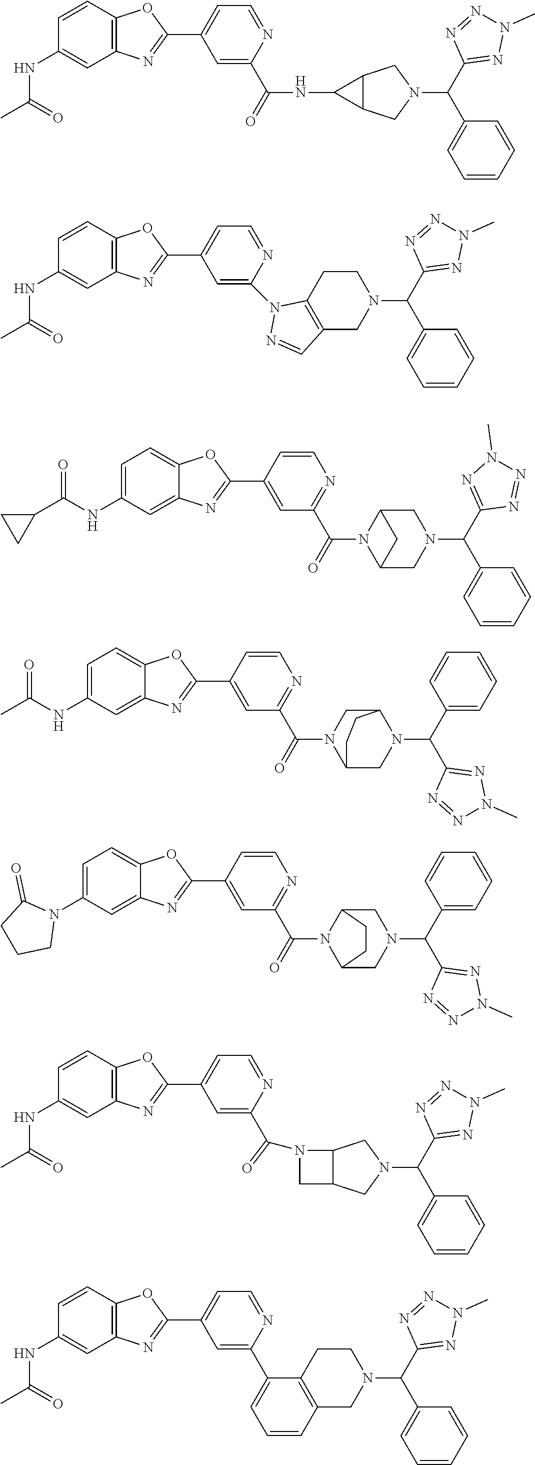

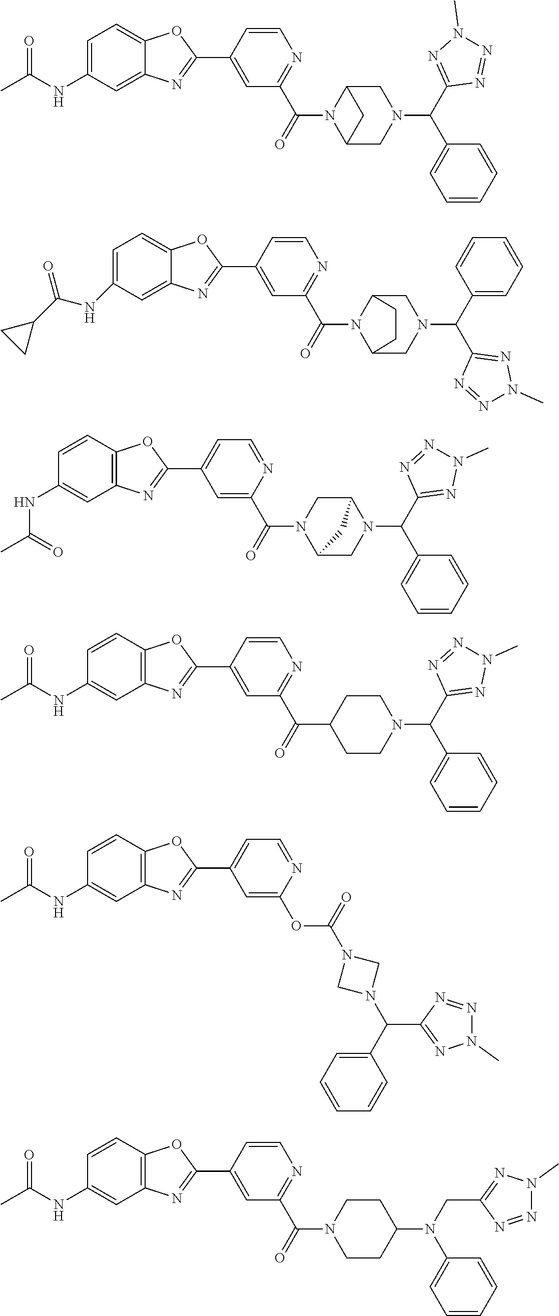

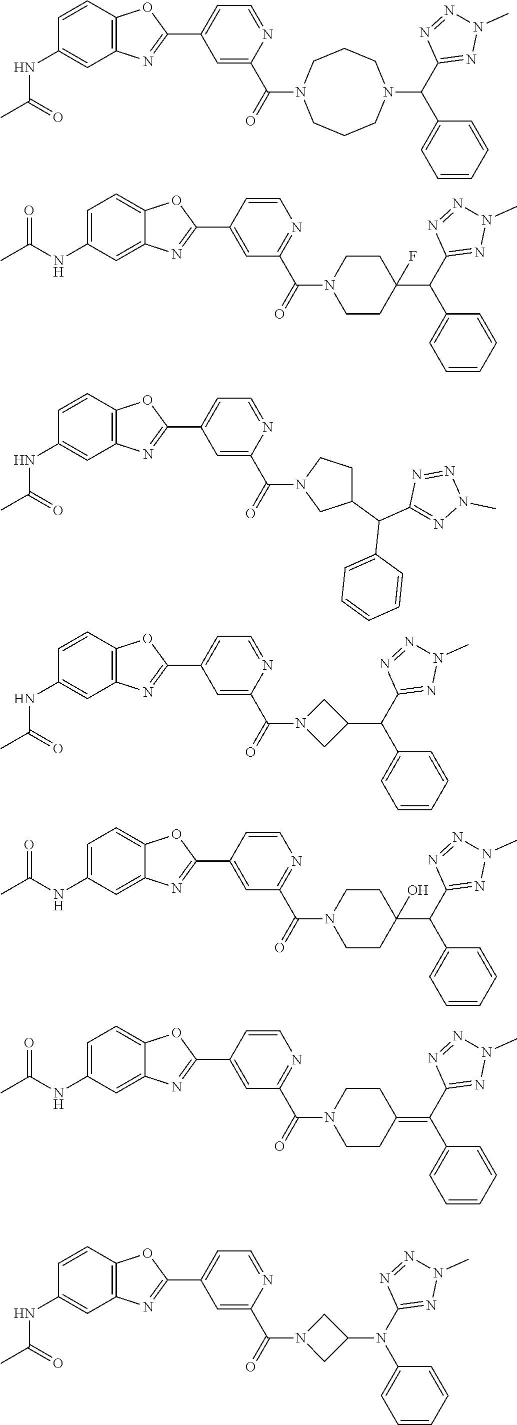

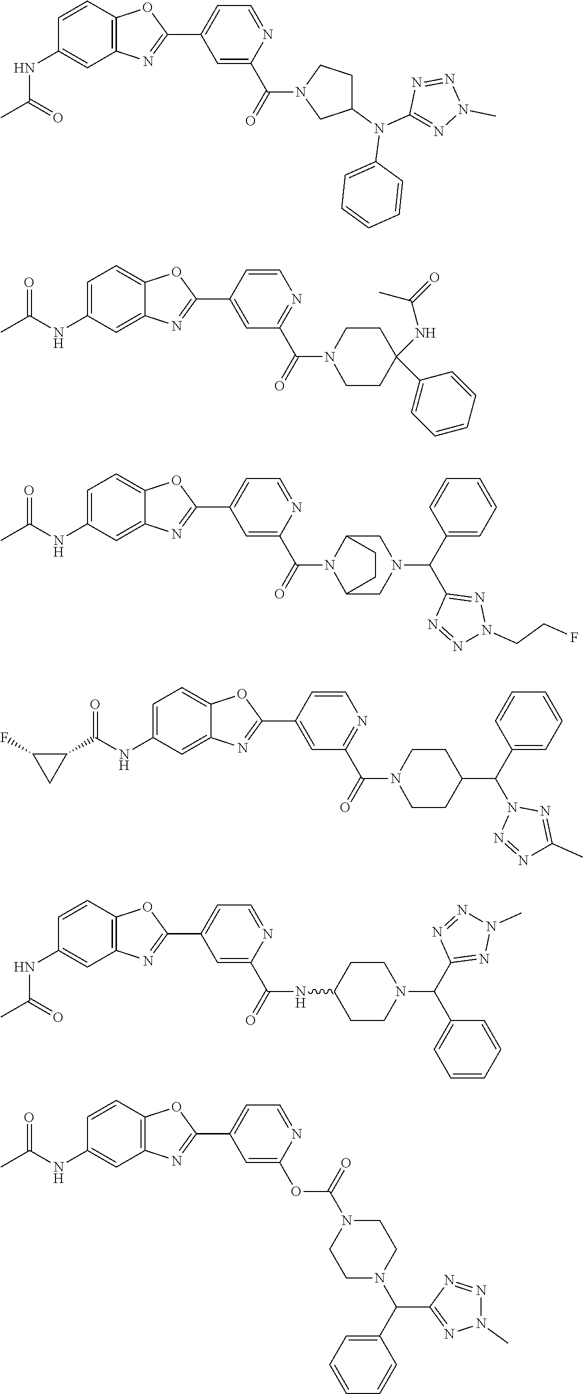

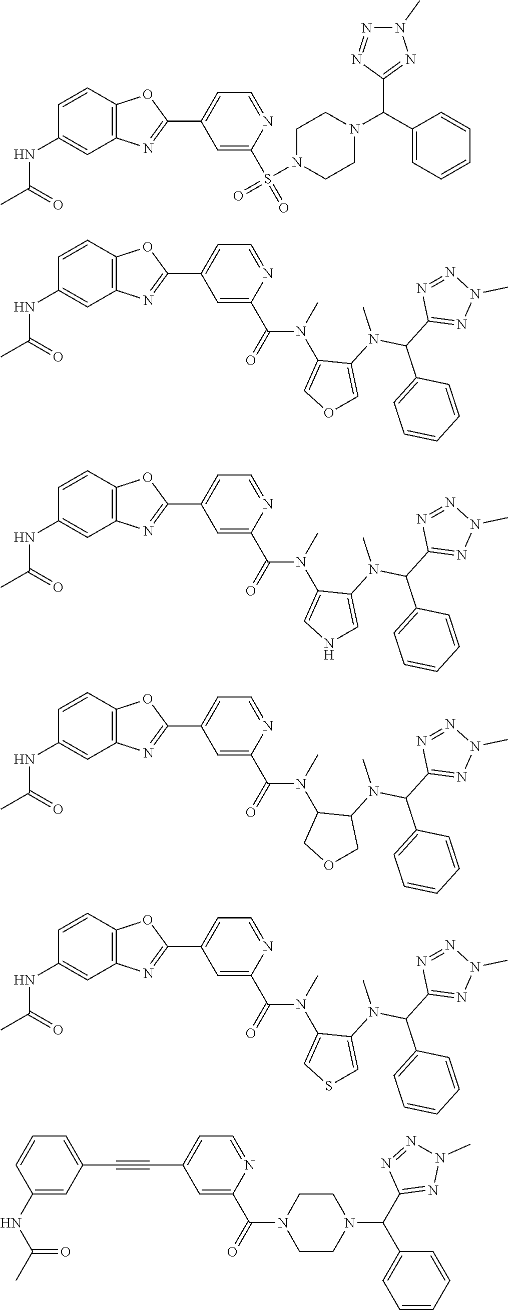

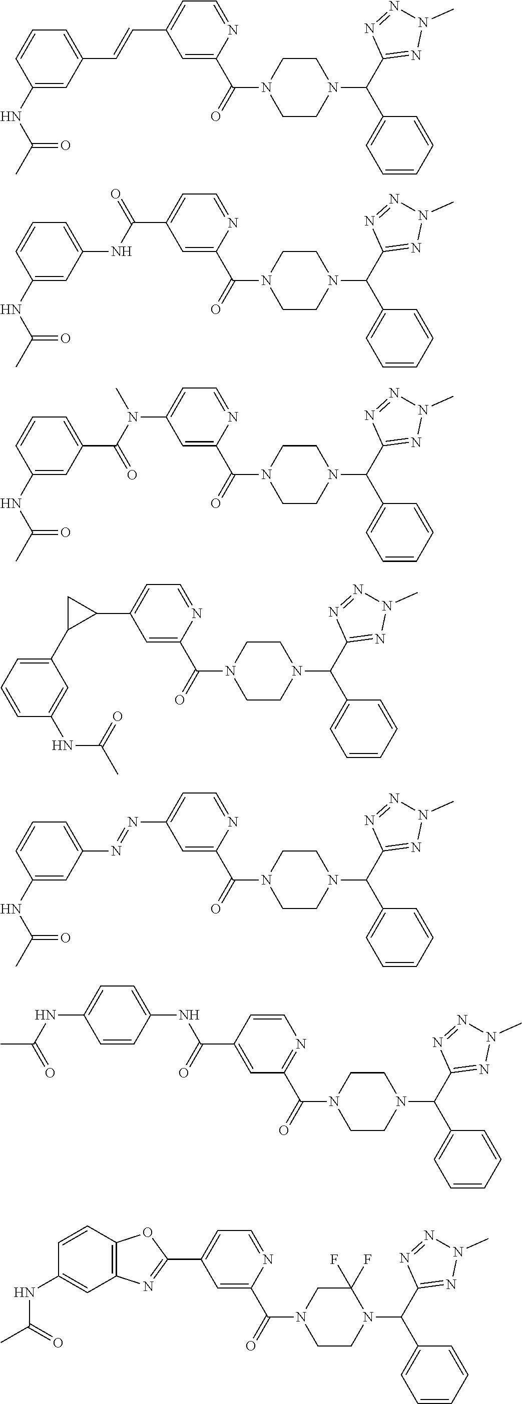

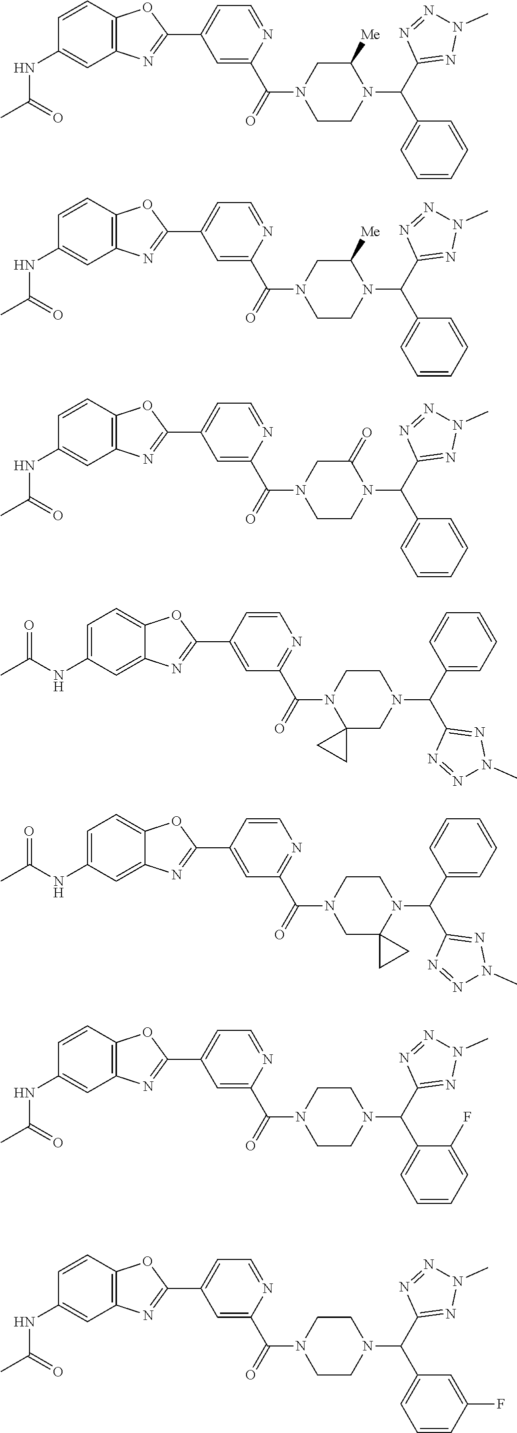

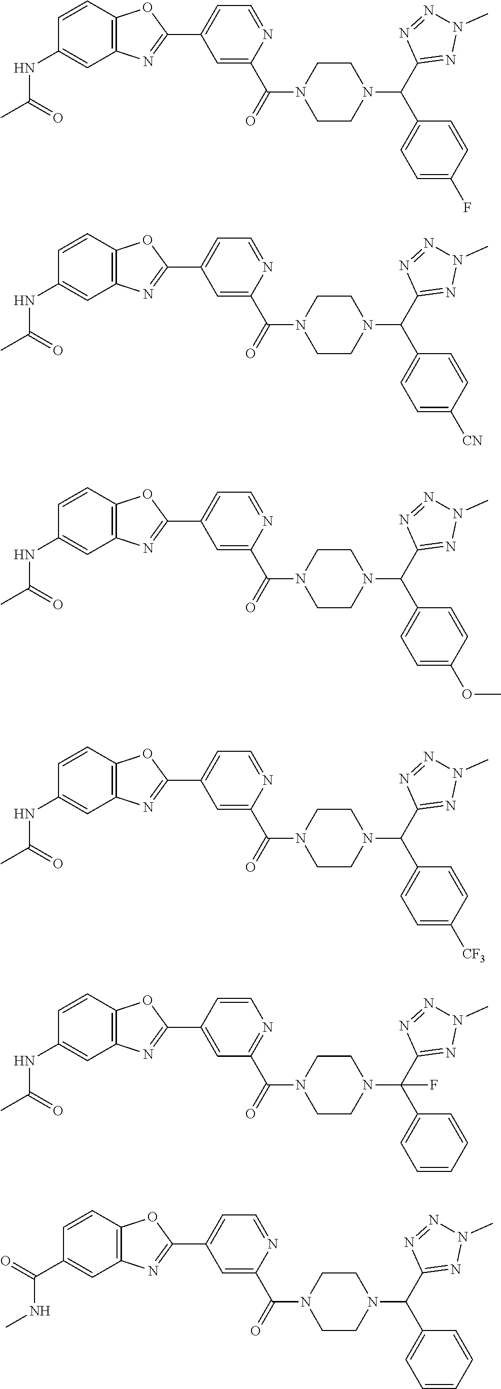

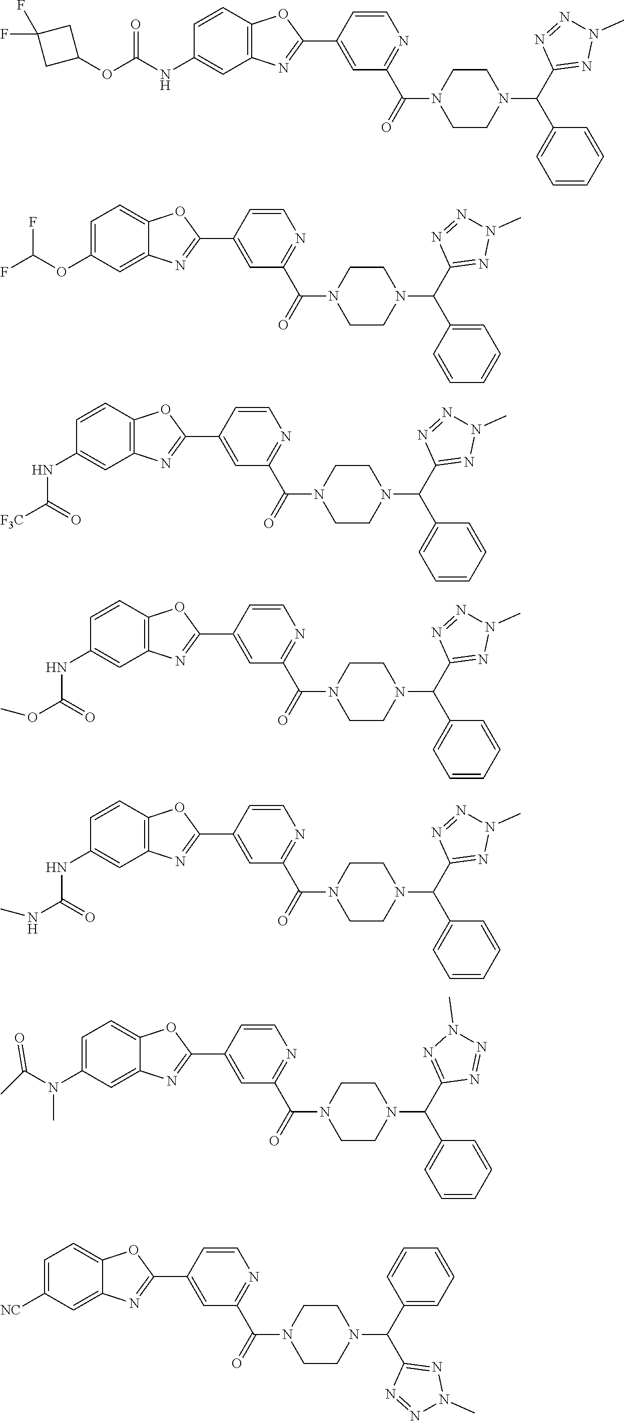

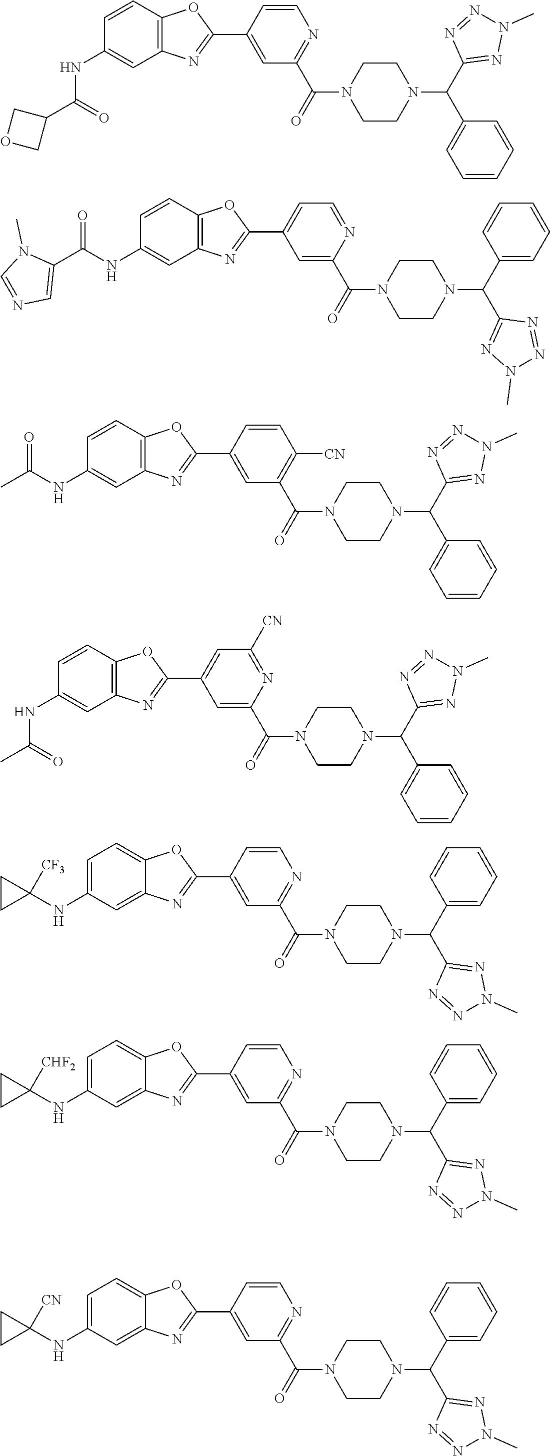

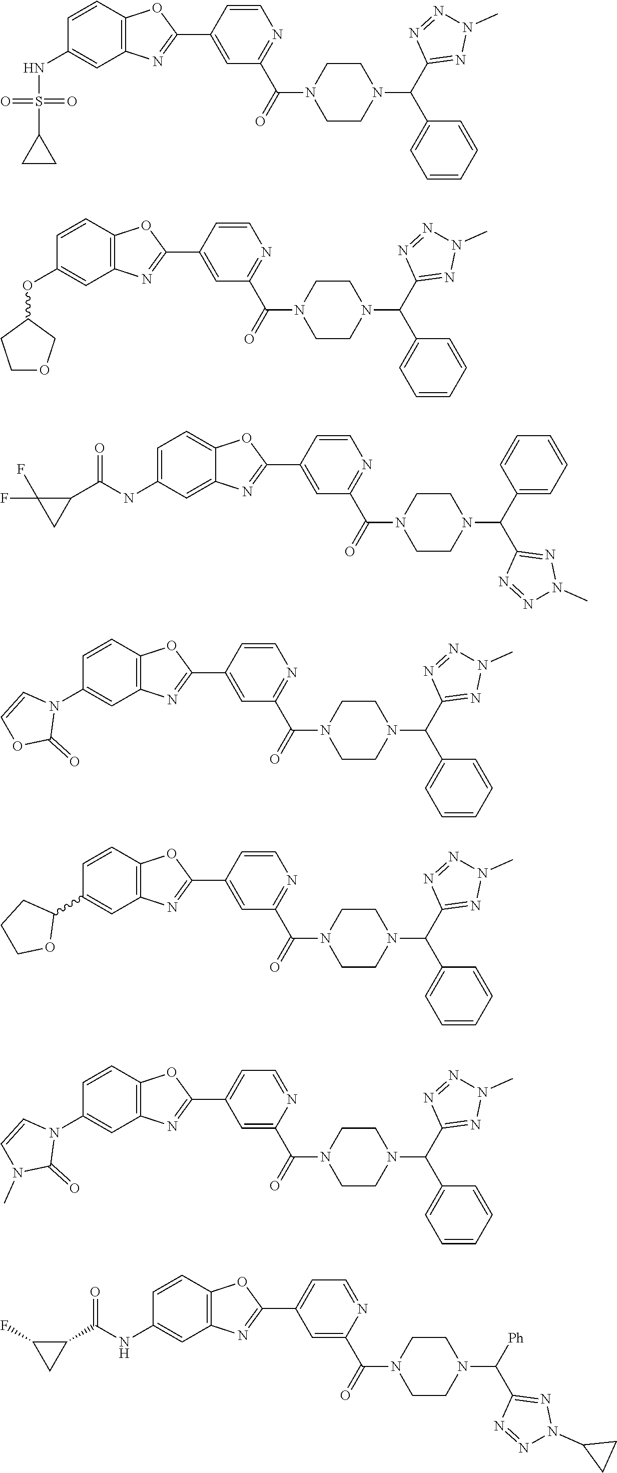

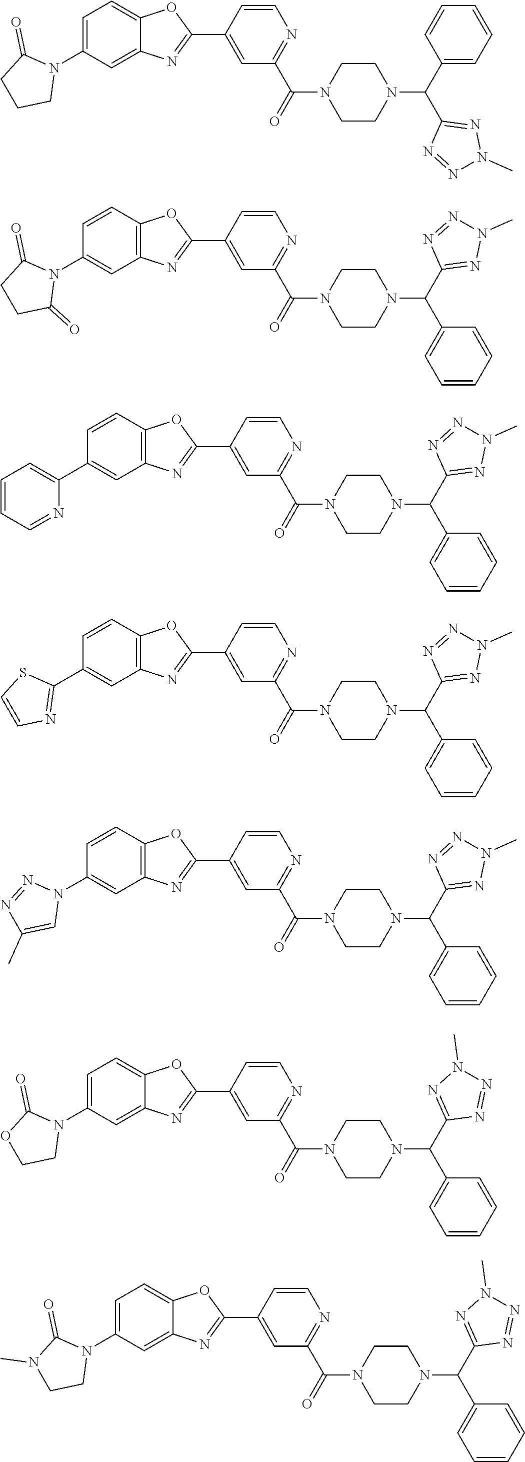

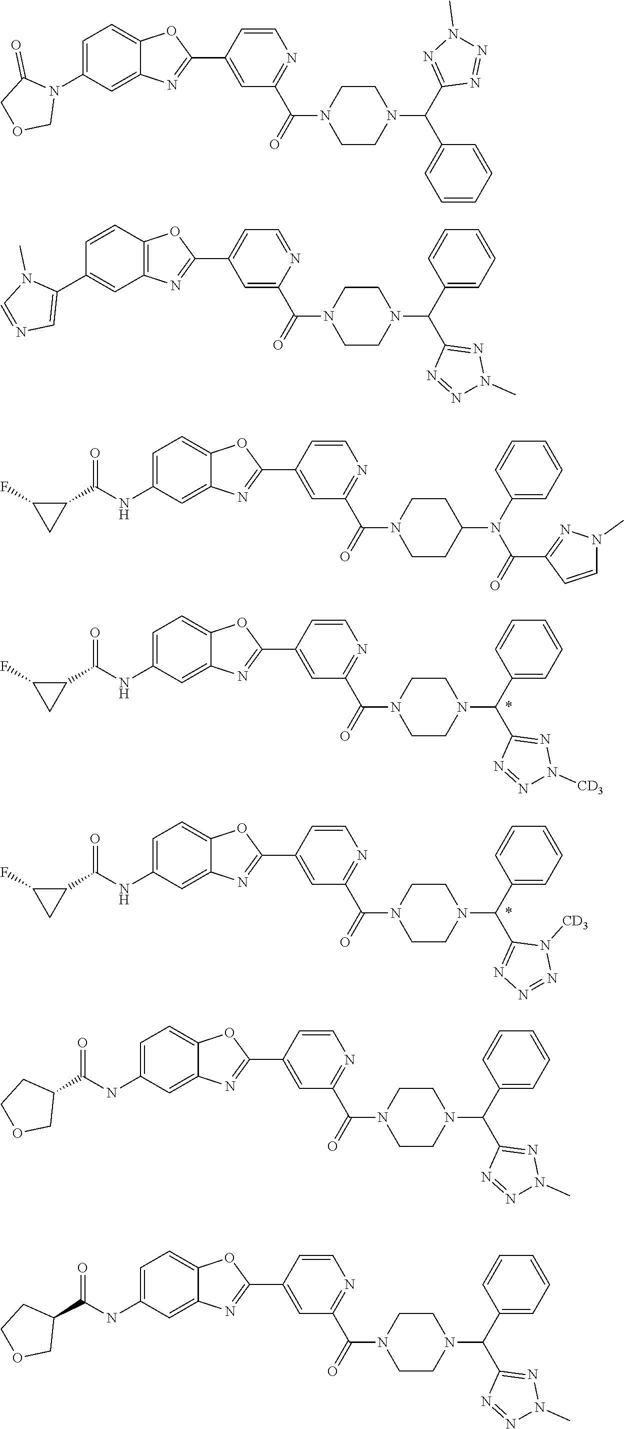

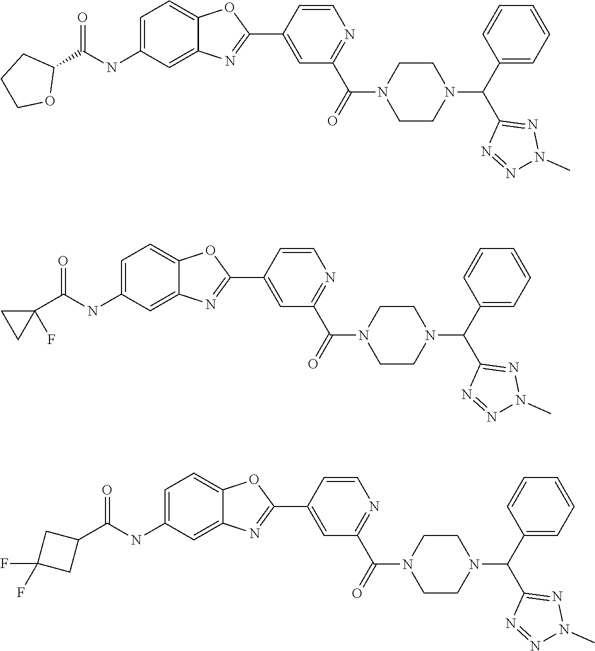 [SMILES: Cn1nnc(C(c2ccccc2)N2CCN(C(=O)c3cc(-c4nc5cc(NC(=O)[C@H]6CCCO6)ccc5o4)ccn3)CC2)n1 ; Cn1nnc(C(c2ccccc2)N2CCN(C(=O)c3cc(-c4nc5cc(NC(=O)C6(F)CC6)ccc5o4)ccn3)CC2)n1 ; Cn1nnc(C(c2ccccc2)N2CCN(C(=O)c3cc(-c4nc5cc(NC(=O)C6CC(F)(F)C6)ccc5o4)ccn3)CC2)n1]

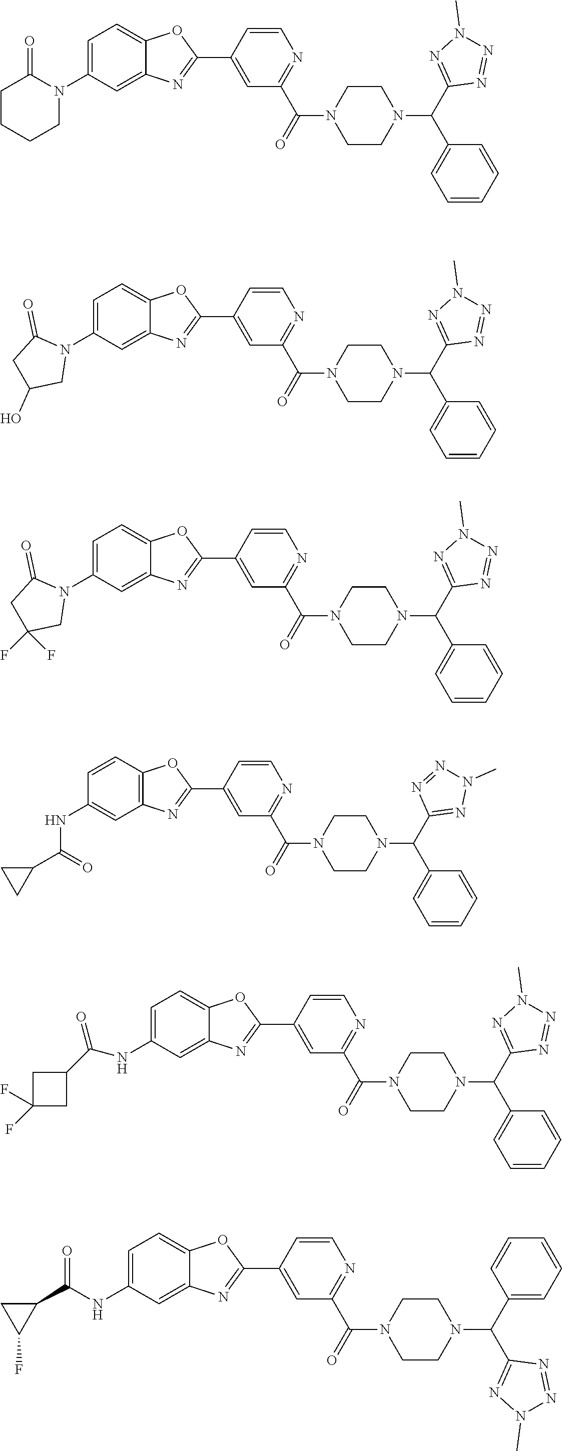

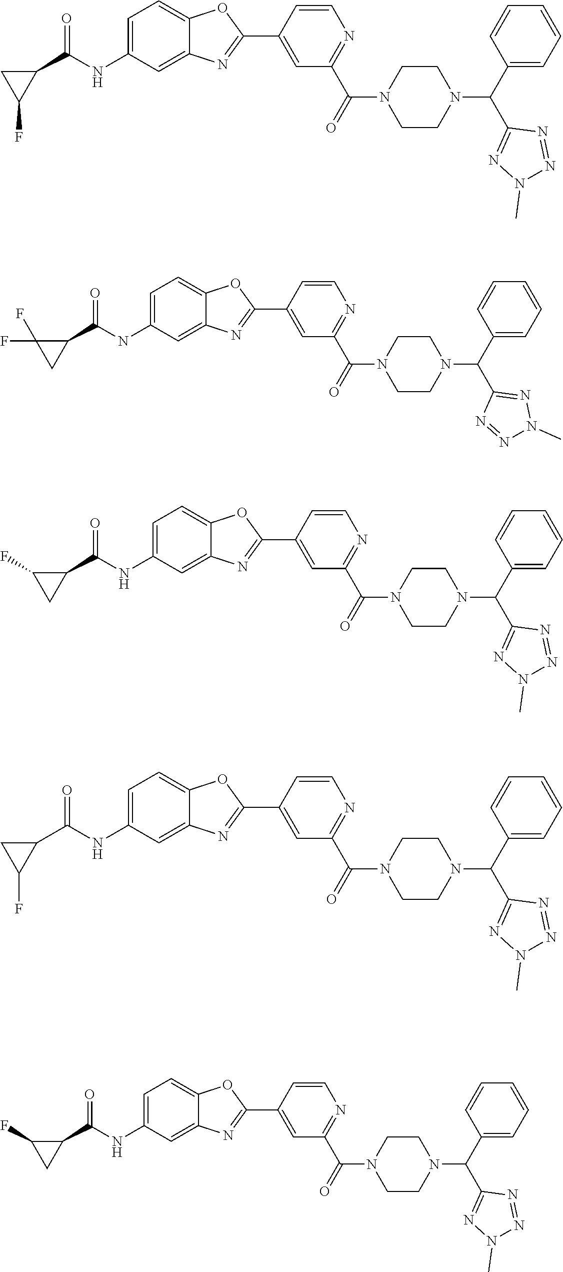 [SMILES: Cn1nnc(C(c2ccccc2)N2CCN(C(=O)c3cc(-c4nc5cc(NC(=O)[C@@H]6C[C@@H]6F)ccc5o4)ccn3)CC2)n1 ; Cn1nnc(C(c2ccccc2)N2CCN(C(=O)c3cc(-c4nc5cc(NC(=O)[C@H]6CC6(F)F)ccc5o4)ccn3)CC2)n1 ; Cn1nnc(C(c2ccccc2)N2CCN(C(=O)c3cc(-c4nc5cc(NC(=O)[C@H]6C[C@@H]6F)ccc5o4)ccn3)CC2)n1 ; Cn1nnc(C(c2ccccc2)N2CCN(C(=O)c3cc(-c4nc5cc(NC(=O)C6CC6F)ccc5o4)ccn3)CC2)n1 ; Cn1nnc(C(c2ccccc2)N2CCN(C(=O)c3cc(-c4nc5cc(NC(=O)[C@H]6C[C@H]6F)ccc5o4)ccn3)CC2)n1]

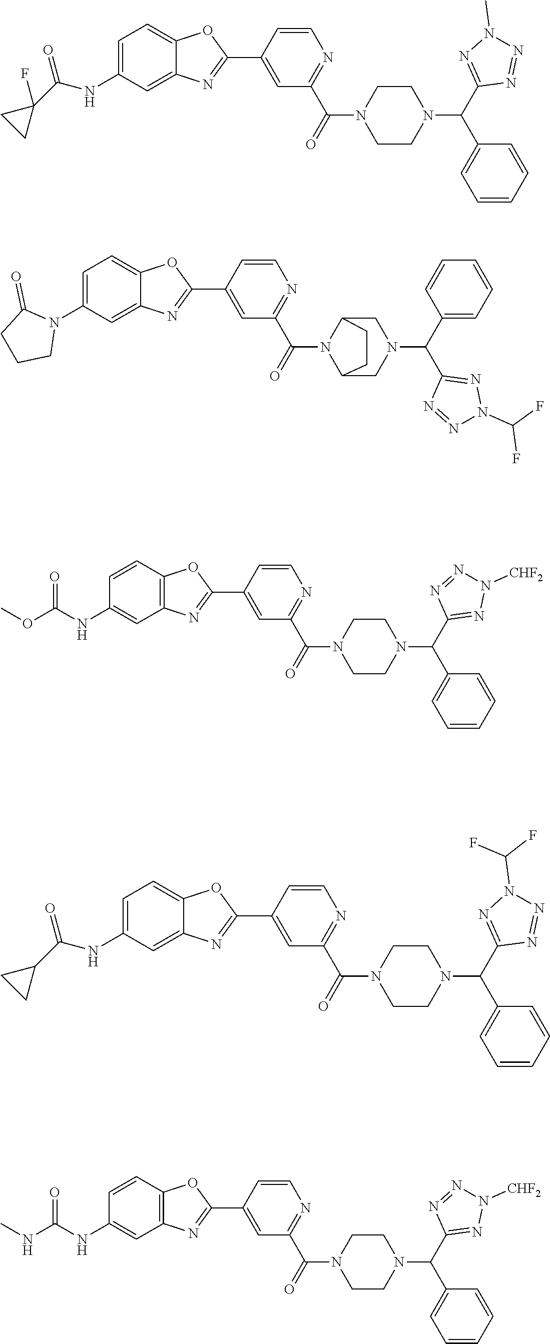 [SMILES: Cn1nnc(C(c2ccccc2)N2CCN(C(=O)c3cc(-c4nc5cc(NC(=O)C6(F)CC6)ccc5o4)ccn3)CC2)n1 ; O=C1CCCN1c1ccc2oc(-c3ccnc(C(=O)N4C5CCC4CN(C(c4ccccc4)c4nnn(C(F)F)n4)C5)c3)nc2c1 ; COC(=O)Nc1ccc2oc(-c3ccnc(C(=O)N4CCN(C(c5ccccc5)c5nnn(C(F)F)n5)CC4)c3)nc2c1 ; O=C(Nc1ccc2oc(-c3ccnc(C(=O)N4CCN(C(c5ccccc5)c5nnn(C(F)F)n5)CC4)c3)nc2c1)C1CC1 ; CNC(=O)Nc1ccc2oc(-c3ccnc(C(=O)N4CCN(C(c5ccccc5)c5nnn(C(F)F)n5)CC4)c3)nc2c1]

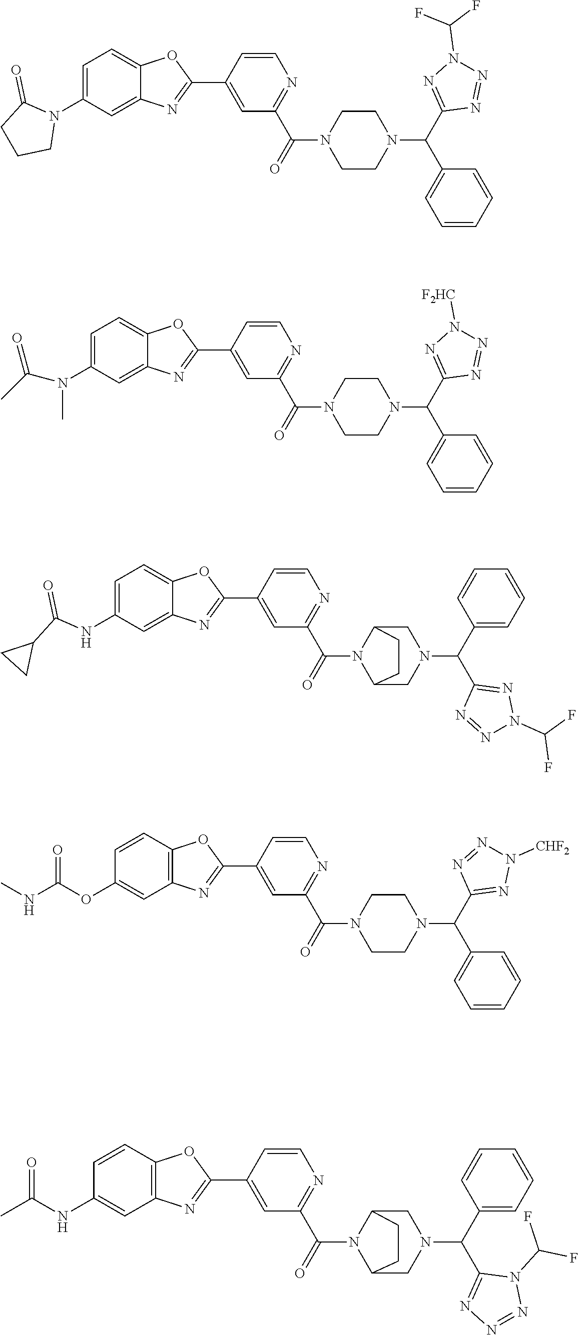 [SMILES: O=C(c1cc(-c2nc3cc(N4CCCC4=O)ccc3o2)ccn1)N1CCN(C(c2ccccc2)c2nnn(C(F)F)n2)CC1 ; CC(=O)N(C)c1ccc2oc(-c3ccnc(C(=O)N4CCN(C(c5ccccc5)c5nnn(C(F)F)n5)CC4)c3)nc2c1 ; O=C(Nc1ccc2oc(-c3ccnc(C(=O)N4C5CCC4CN(C(c4ccccc4)c4nnn(C(F)F)n4)C5)c3)nc2c1)C1CC1 ; CNC(=O)Oc1ccc2oc(-c3ccnc(C(=O)N4CCN(C(c5ccccc5)c5nnn(C(F)F)n5)CC4)c3)nc2c1 ; CC(=O)Nc1ccc2oc(-c3ccnc(C(=O)N4C5CCC4CN(C(c4ccccc4)c4nnnn4C(F)F)C5)c3)nc2c1]

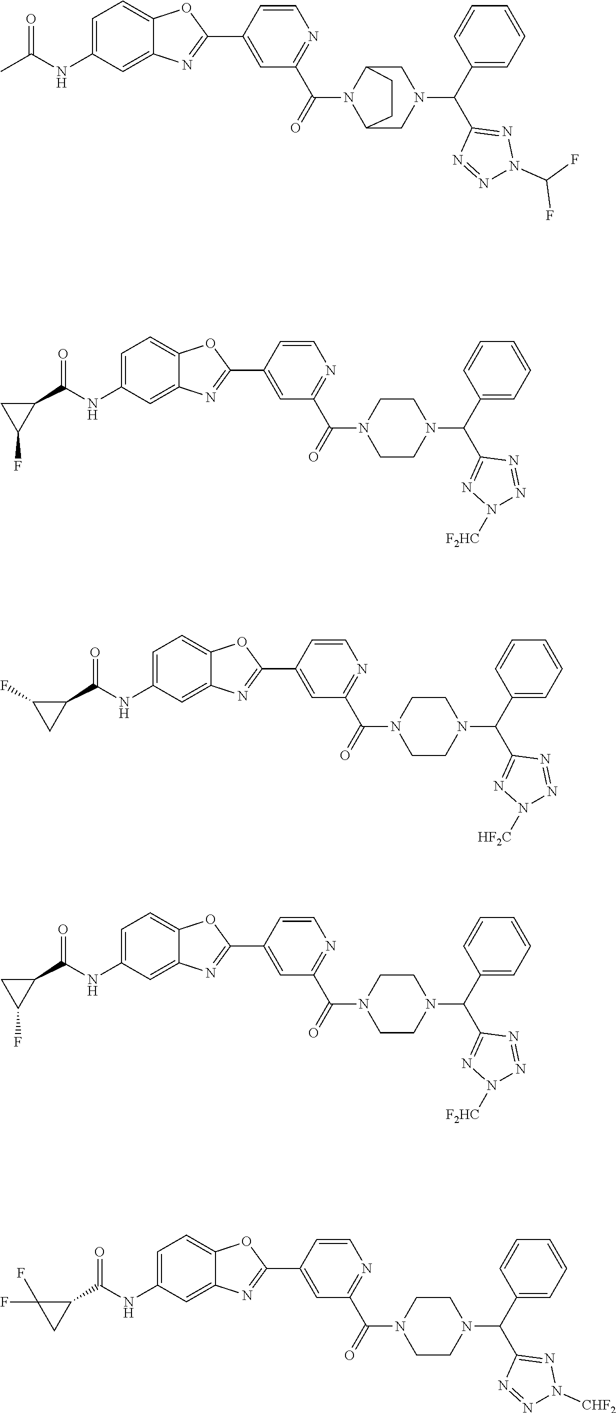

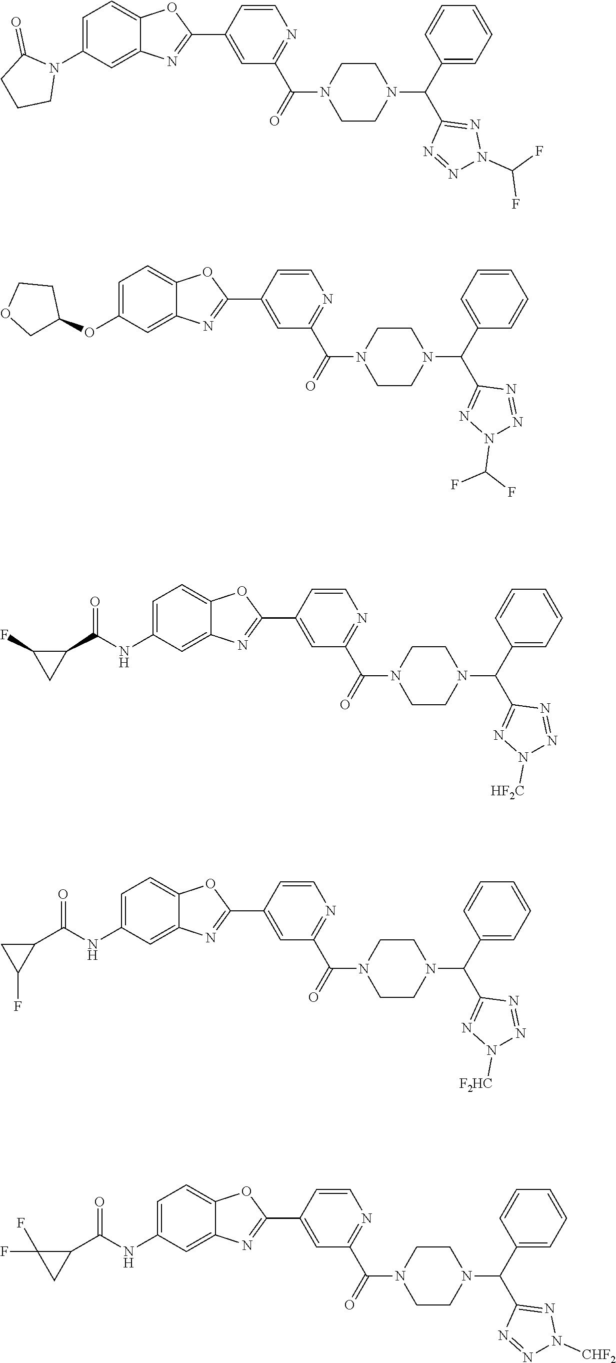

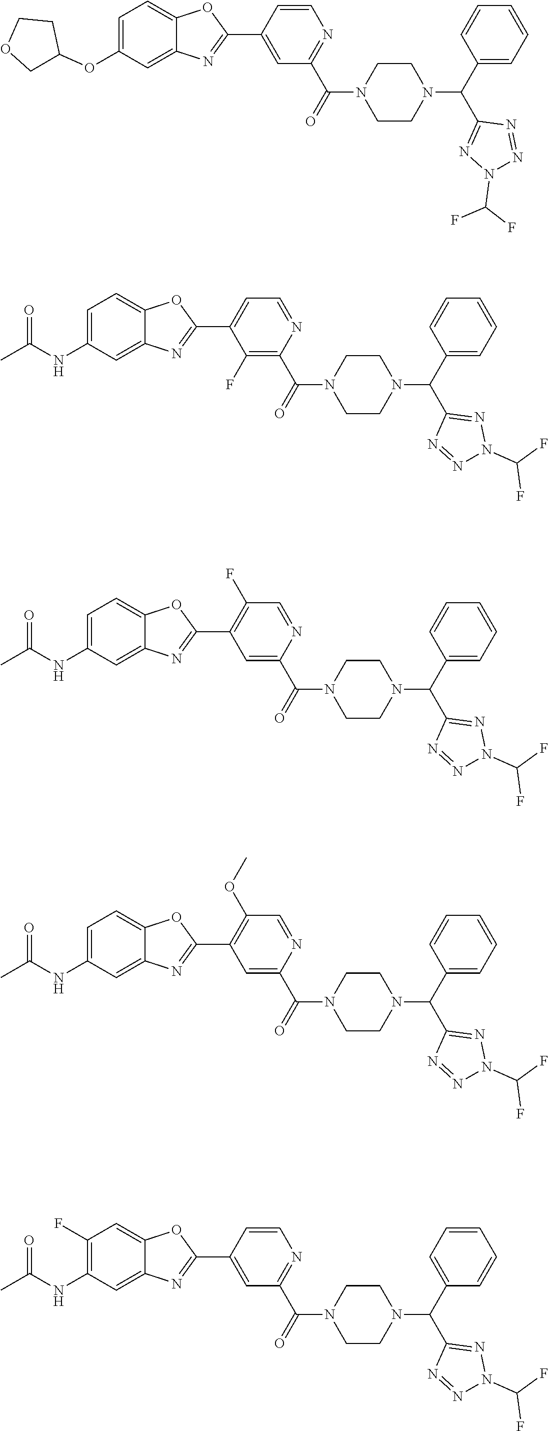 [SMILES: O=C(c1cc(-c2nc3cc(OC4CCOC4)ccc3o2)ccn1)N1CCN(C(c2ccccc2)c2nnn(C(F)F)n2)CC1 ; CC(=O)Nc1ccc2oc(-c3ccnc(C(=O)N4CCN(C(c5ccccc5)c5nnn(C(F)F)n5)CC4)c3F)nc2c1 ; CC(=O)Nc1ccc2oc(-c3cc(C(=O)N4CCN(C(c5ccccc5)c5nnn(C(F)F)n5)CC4)ncc3F)nc2c1 ; COc1cnc(C(=O)N2CCN(C(c3ccccc3)c3nnn(C(F)F)n3)CC2)cc1-c1nc2cc(NC(C)=O)ccc2o1 ; CC(=O)Nc1cc2nc(-c3ccnc(C(=O)N4CCN(C(c5ccccc5)c5nnn(C(F)F)n5)CC4)c3)oc2cc1F]

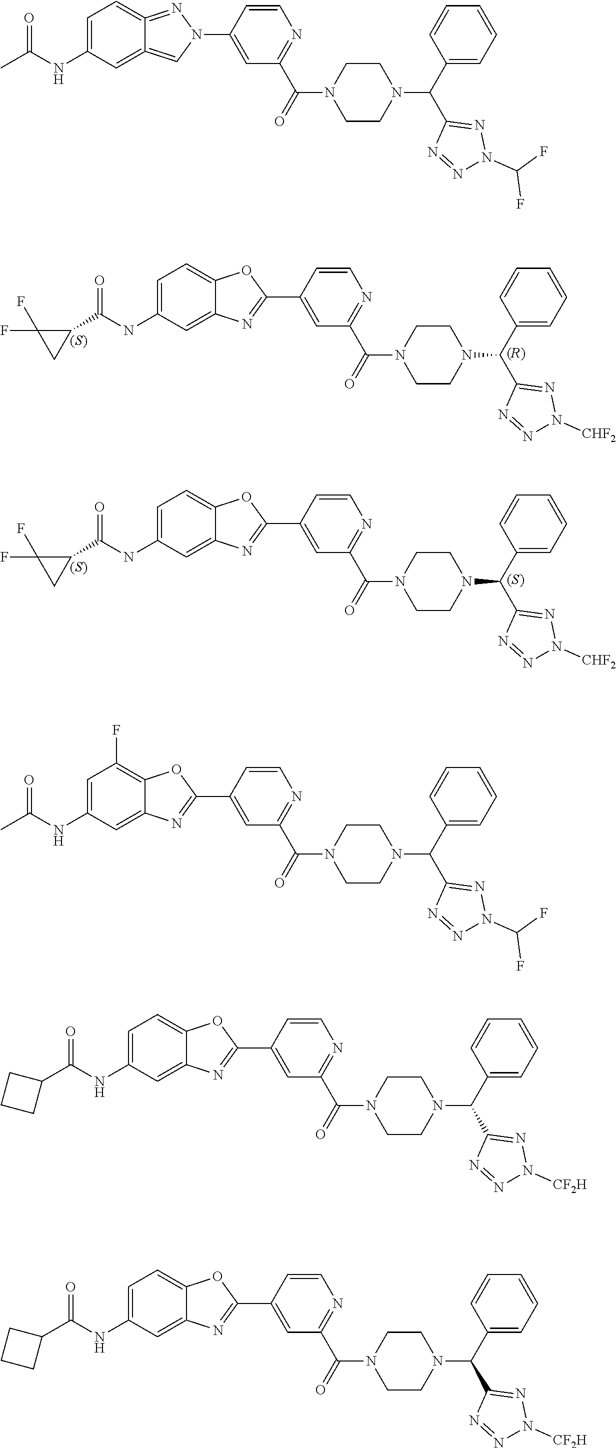

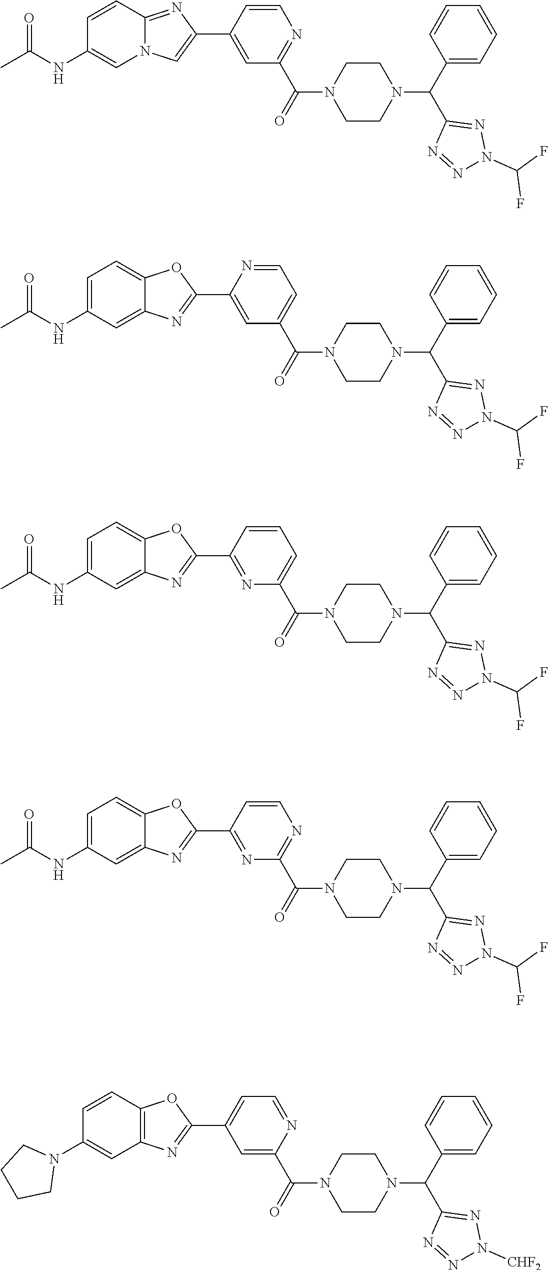 [SMILES: CC(=O)Nc1ccc2nc(-c3ccnc(C(=O)N4CCN(C(c5ccccc5)c5nnn(C(F)F)n5)CC4)c3)cn2c1 ; CC(=O)Nc1ccc2oc(-c3cc(C(=O)N4CCN(C(c5ccccc5)c5nnn(C(F)F)n5)CC4)ccn3)nc2c1 ; CC(=O)Nc1ccc2oc(-c3cccc(C(=O)N4CCN(C(c5ccccc5)c5nnn(C(F)F)n5)CC4)n3)nc2c1 ; CC(=O)Nc1ccc2oc(-c3ccnc(C(=O)N4CCN(C(c5ccccc5)c5nnn(C(F)F)n5)CC4)n3)nc2c1 ; O=C(c1cc(-c2nc3cc(N4CCCC4)ccc3o2)ccn1)N1CCN(C(c2ccccc2)c2nnn(C(F)F)n2)CC1]

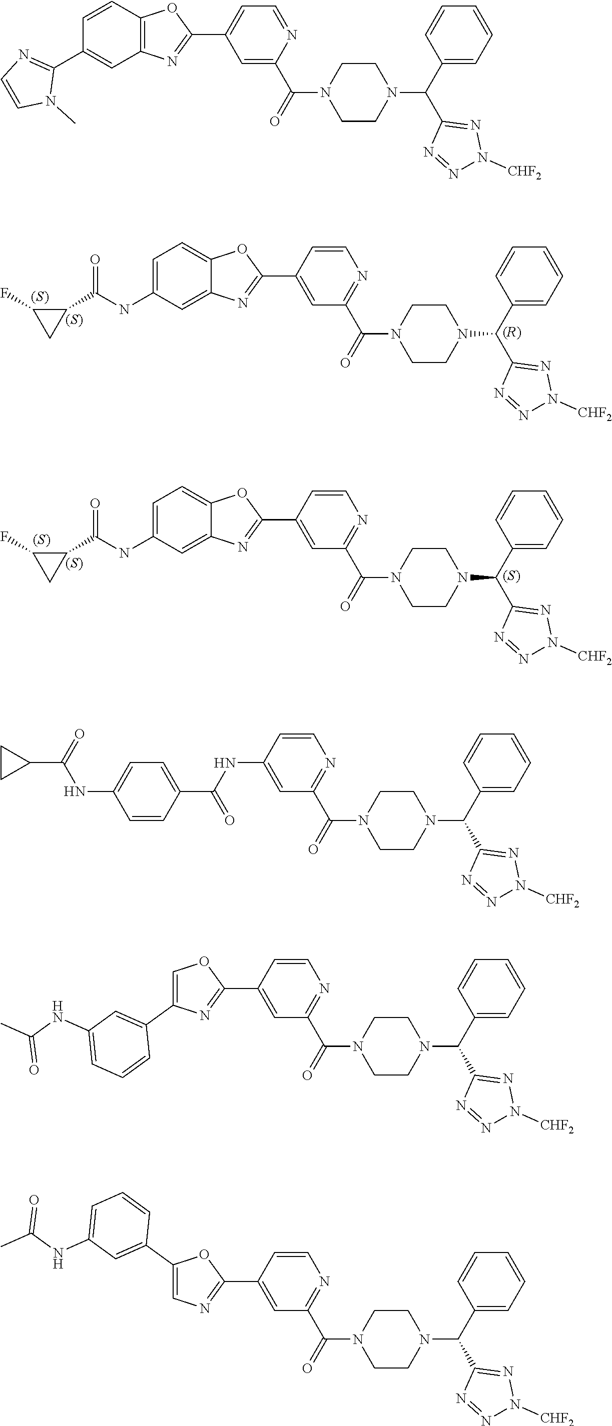

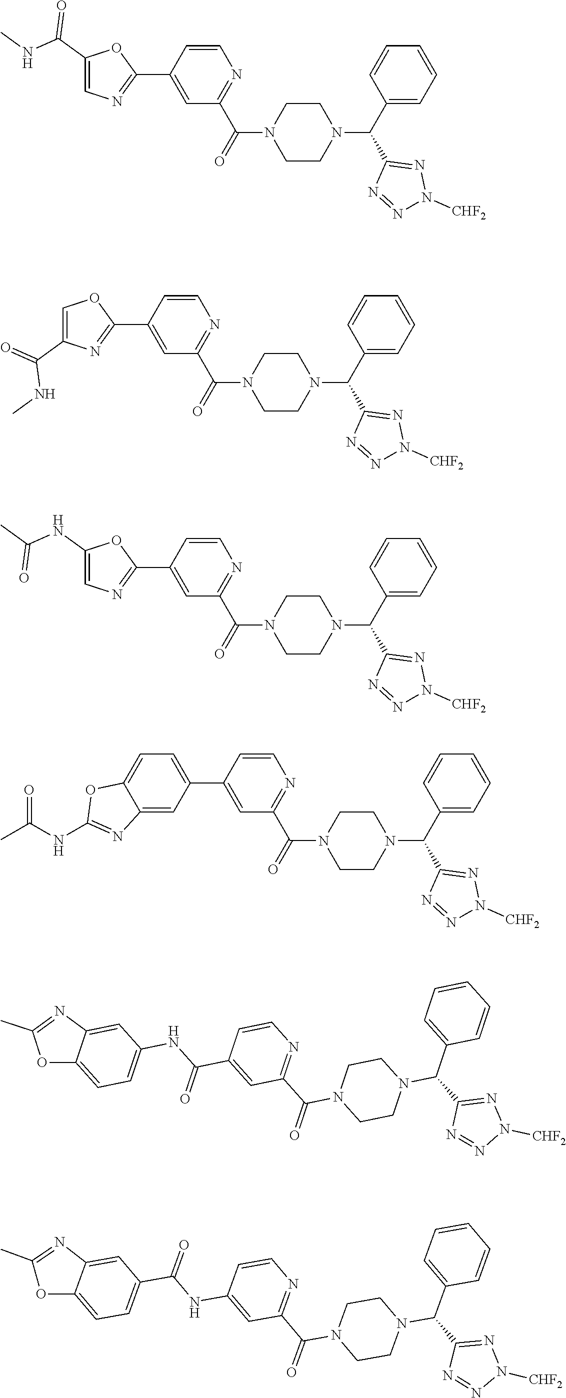 [SMILES: CNC(=O)c1cnc(-c2ccnc(C(=O)N3CCN([C@H](c4ccccc4)c4nnn(C(F)F)n4)CC3)c2)o1 ; CNC(=O)c1coc(-c2ccnc(C(=O)N3CCN([C@H](c4ccccc4)c4nnn(C(F)F)n4)CC3)c2)n1 ; CC(=O)Nc1cnc(-c2ccnc(C(=O)N3CCN([C@H](c4ccccc4)c4nnn(C(F)F)n4)CC3)c2)o1 ; CC(=O)Nc1nc2cc(-c3ccnc(C(=O)N4CCN([C@H](c5ccccc5)c5nnn(C(F)F)n5)CC4)c3)ccc2o1 ; Cc1nc2cc(NC(=O)c3ccnc(C(=O)N4CCN([C@H](c5ccccc5)c5nnn(C(F)F)n5)CC4)c3)ccc2o1 ; Cc1nc2cc(C(=O)Nc3ccnc(C(=O)N4CCN([C@H](c5ccccc5)c5nnn(C(F)F)n5)CC4)c3)ccc2o1]

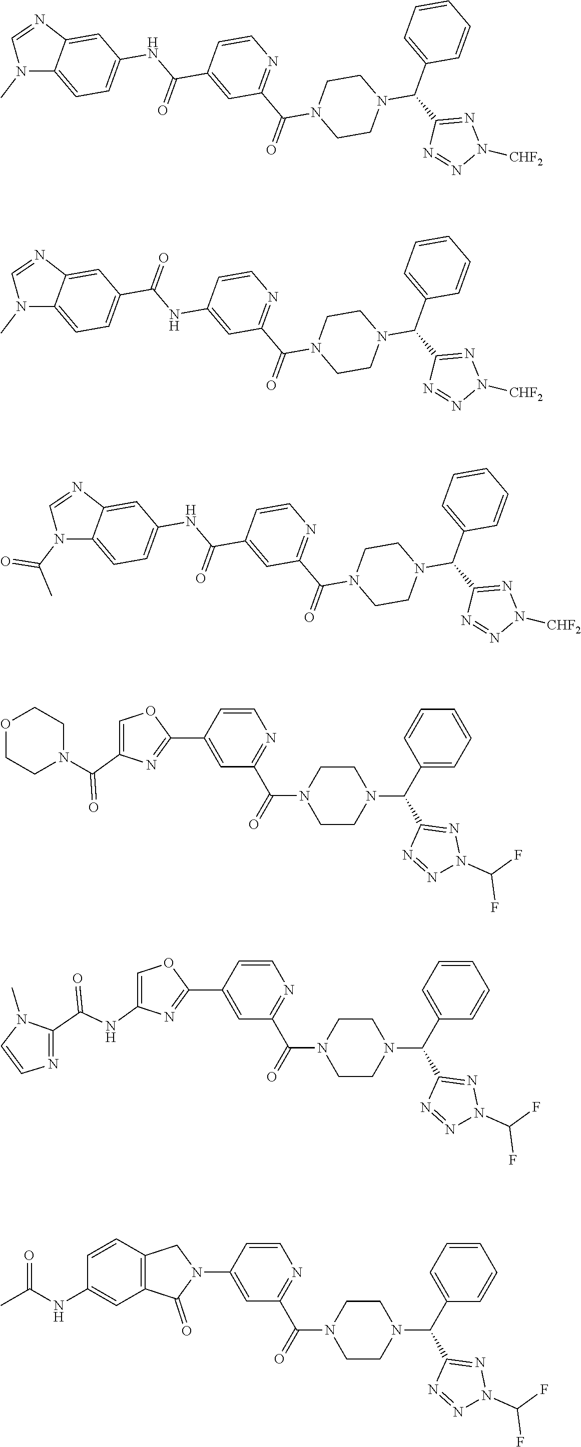 [SMILES: Cn1cnc2cc(NC(=O)c3ccnc(C(=O)N4CCN([C@H](c5ccccc5)c5nnn(C(F)F)n5)CC4)c3)ccc21 ; Cn1cnc2cc(C(=O)Nc3ccnc(C(=O)N4CCN([C@H](c5ccccc5)c5nnn(C(F)F)n5)CC4)c3)ccc21 ; CC(=O)n1cnc2cc(NC(=O)c3ccnc(C(=O)N4CCN([C@H](c5ccccc5)c5nnn(C(F)F)n5)CC4)c3)ccc21 ; O=C(c1cc(-c2nc(C(=O)N3CCOCC3)co2)ccn1)N1CCN([C@H](c2ccccc2)c2nnn(C(F)F)n2)CC1 ; Cn1ccnc1C(=O)Nc1coc(-c2ccnc(C(=O)N3CCN([C@H](c4ccccc4)c4nnn(C(F)F)n4)CC3)c2)n1 ; CC(=O)Nc1ccc2c(c1)C(=O)N(c1ccnc(C(=O)N3CCN([C@H](c4ccccc4)c4nnn(C(F)F)n4)CC3)c1)C2]

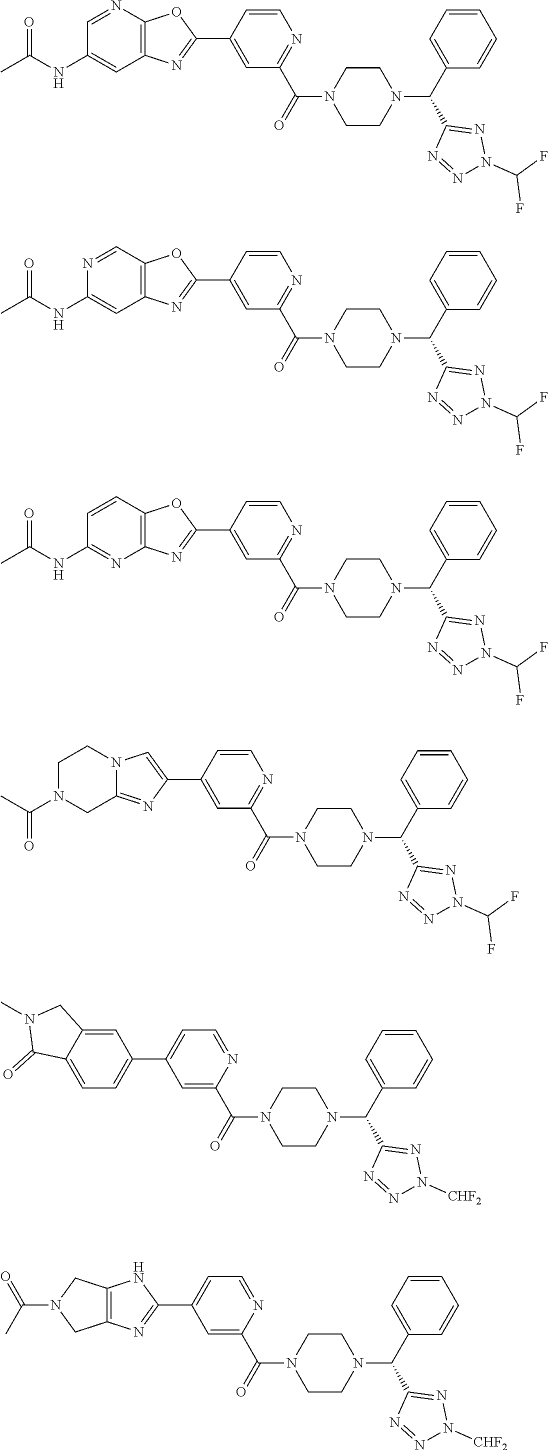 [SMILES: CC(=O)Nc1cnc2oc(-c3ccnc(C(=O)N4CCN([C@H](c5ccccc5)c5nnn(C(F)F)n5)CC4)c3)nc2c1 ; CC(=O)Nc1cc2nc(-c3ccnc(C(=O)N4CCN([C@H](c5ccccc5)c5nnn(C(F)F)n5)CC4)c3)oc2cn1 ; CC(=O)Nc1ccc2oc(-c3ccnc(C(=O)N4CCN([C@H](c5ccccc5)c5nnn(C(F)F)n5)CC4)c3)nc2n1 ; CC(=O)N1CCn2cc(-c3ccnc(C(=O)N4CCN([C@H](c5ccccc5)c5nnn(C(F)F)n5)CC4)c3)nc2C1 ; CN1Cc2cc(-c3ccnc(C(=O)N4CCN([C@H](c5ccccc5)c5nnn(C(F)F)n5)CC4)c3)ccc2C1=O ; CC(=O)N1Cc2nc(-c3ccnc(C(=O)N4CCN([C@H](c5ccccc5)c5nnn(C(F)F)n5)CC4)c3)[nH]c2C1]

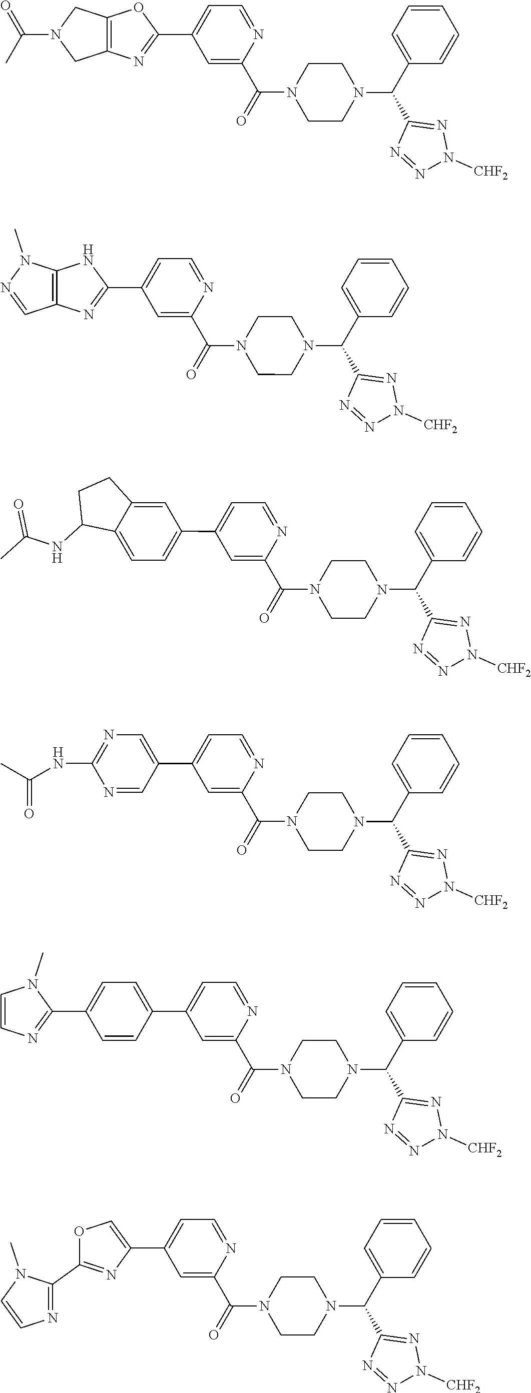 [SMILES: CC(=O)N1Cc2nc(-c3ccnc(C(=O)N4CCN([C@H](c5ccccc5)c5nnn(C(F)F)n5)CC4)c3)oc2C1 ; Cn1ncc2nc(-c3ccnc(C(=O)N4CCN([C@H](c5ccccc5)c5nnn(C(F)F)n5)CC4)c3)[nH]c21 ; CC(=O)NC1CCc2cc(-c3ccnc(C(=O)N4CCN([C@H](c5ccccc5)c5nnn(C(F)F)n5)CC4)c3)ccc21 ; CC(=O)Nc1ncc(-c2ccnc(C(=O)N3CCN([C@H](c4ccccc4)c4nnn(C(F)F)n4)CC3)c2)cn1 ; Cn1ccnc1-c1ccc(-c2ccnc(C(=O)N3CCN([C@H](c4ccccc4)c4nnn(C(F)F)n4)CC3)c2)cc1 ; Cn1ccnc1-c1nc(-c2ccnc(C(=O)N3CCN([C@H](c4ccccc4)c4nnn(C(F)F)n4)CC3)c2)co1]

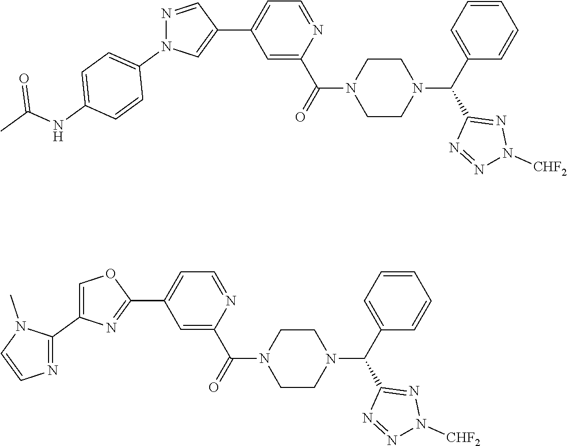 [SMILES: CC(=O)Nc1ccc(-n2cc(-c3ccnc(C(=O)N4CCN([C@H](c5ccccc5)c5nnn(C(F)F)n5)CC4)c3)cn2)cc1 ; Cn1ccnc1-c1coc(-c2ccnc(C(=O)N3CCN([C@H](c4ccccc4)c4nnn(C(F)F)n4)CC3)c2)n1]

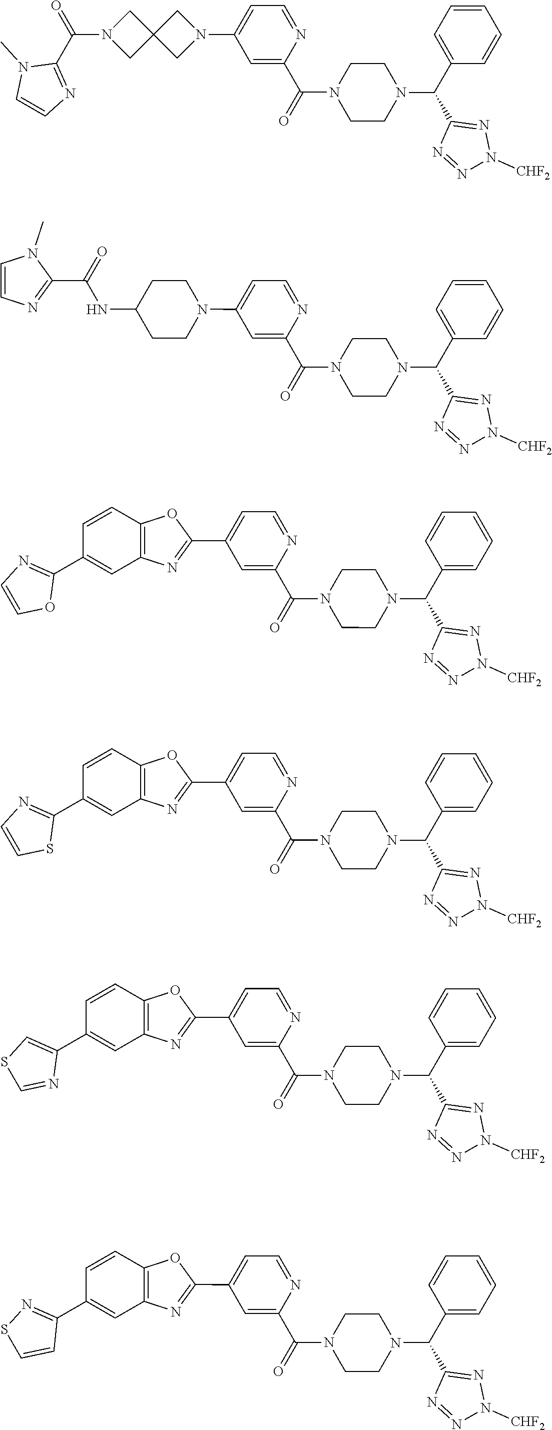 [SMILES: Cn1ccnc1C(=O)N1CC2(C1)CN(c1ccnc(C(=O)N3CCN([C@H](c4ccccc4)c4nnn(C(F)F)n4)CC3)c1)C2 ; Cn1ccnc1C(=O)NC1CCN(c2ccnc(C(=O)N3CCN([C@H](c4ccccc4)c4nnn(C(F)F)n4)CC3)c2)CC1 ; O=C(c1cc(-c2nc3cc(-c4ncco4)ccc3o2)ccn1)N1CCN([C@H](c2ccccc2)c2nnn(C(F)F)n2)CC1 ; O=C(c1cc(-c2nc3cc(-c4nccs4)ccc3o2)ccn1)N1CCN([C@H](c2ccccc2)c2nnn(C(F)F)n2)CC1 ; O=C(c1cc(-c2nc3cc(-c4cscn4)ccc3o2)ccn1)N1CCN([C@H](c2ccccc2)c2nnn(C(F)F)n2)CC1 ; O=C(c1cc(-c2nc3cc(-c4ccsn4)ccc3o2)ccn1)N1CCN([C@H](c2ccccc2)c2nnn(C(F)F)n2)CC1]

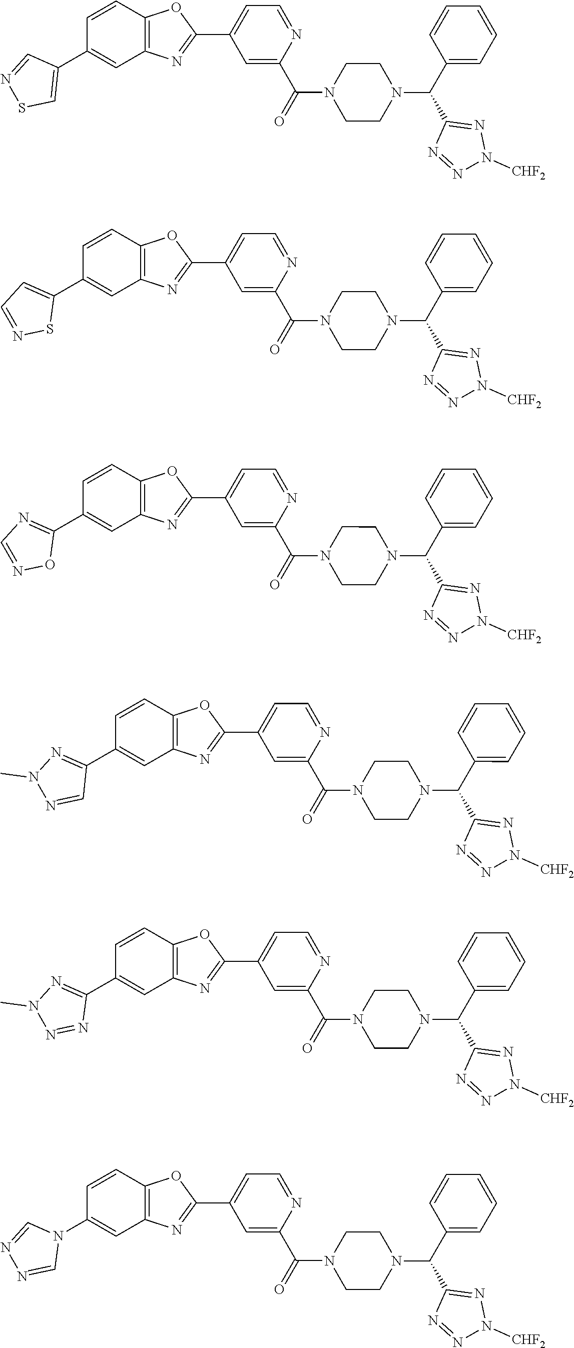 [SMILES: O=C(c1cc(-c2nc3cc(-c4cnsc4)ccc3o2)ccn1)N1CCN([C@H](c2ccccc2)c2nnn(C(F)F)n2)CC1 ; O=C(c1cc(-c2nc3cc(-c4ccns4)ccc3o2)ccn1)N1CCN([C@H](c2ccccc2)c2nnn(C(F)F)n2)CC1 ; O=C(c1cc(-c2nc3cc(-c4ncno4)ccc3o2)ccn1)N1CCN([C@H](c2ccccc2)c2nnn(C(F)F)n2)CC1 ; Cn1ncc(-c2ccc3oc(-c4ccnc(C(=O)N5CCN([C@H](c6ccccc6)c6nnn(C(F)F)n6)CC5)c4)nc3c2)n1 ; Cn1nnc(-c2ccc3oc(-c4ccnc(C(=O)N5CCN([C@H](c6ccccc6)c6nnn(C(F)F)n6)CC5)c4)nc3c2)n1 ; O=C(c1cc(-c2nc3cc(-n4cnnc4)ccc3o2)ccn1)N1CCN([C@H](c2ccccc2)c2nnn(C(F)F)n2)CC1]

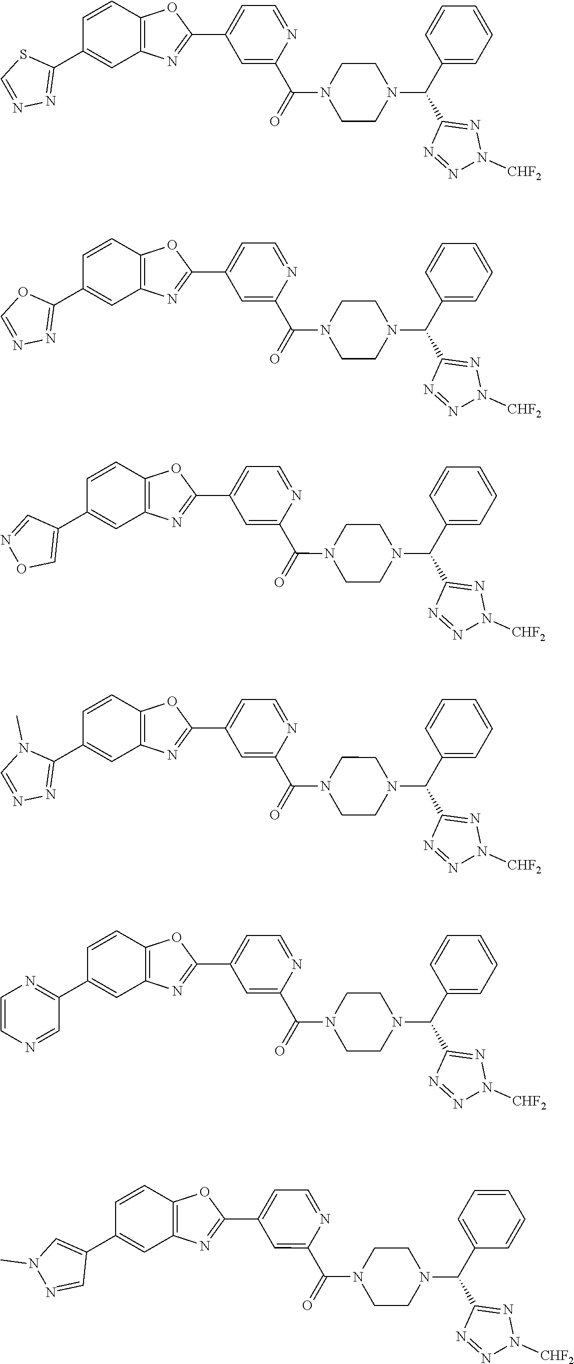 [SMILES: O=C(c1cc(-c2nc3cc(-c4nncs4)ccc3o2)ccn1)N1CCN([C@H](c2ccccc2)c2nnn(C(F)F)n2)CC1 ; O=C(c1cc(-c2nc3cc(-c4nnco4)ccc3o2)ccn1)N1CCN([C@H](c2ccccc2)c2nnn(C(F)F)n2)CC1 ; O=C(c1cc(-c2nc3cc(-c4cnoc4)ccc3o2)ccn1)N1CCN([C@H](c2ccccc2)c2nnn(C(F)F)n2)CC1 ; Cn1cnnc1-c1ccc2oc(-c3ccnc(C(=O)N4CCN([C@H](c5ccccc5)c5nnn(C(F)F)n5)CC4)c3)nc2c1 ; O=C(c1cc(-c2nc3cc(-c4cnccn4)ccc3o2)ccn1)N1CCN([C@H](c2ccccc2)c2nnn(C(F)F)n2)CC1 ; Cn1cc(-c2ccc3oc(-c4ccnc(C(=O)N5CCN([C@H](c6ccccc6)c6nnn(C(F)F)n6)CC5)c4)nc3c2)cn1]

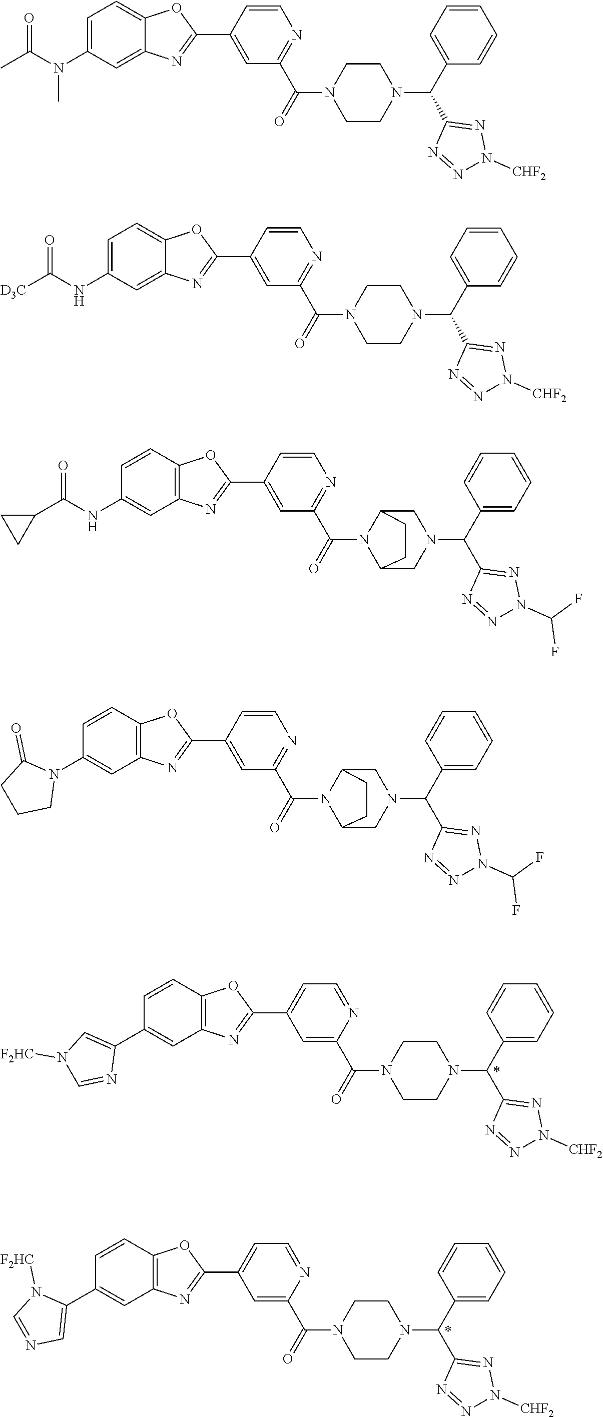

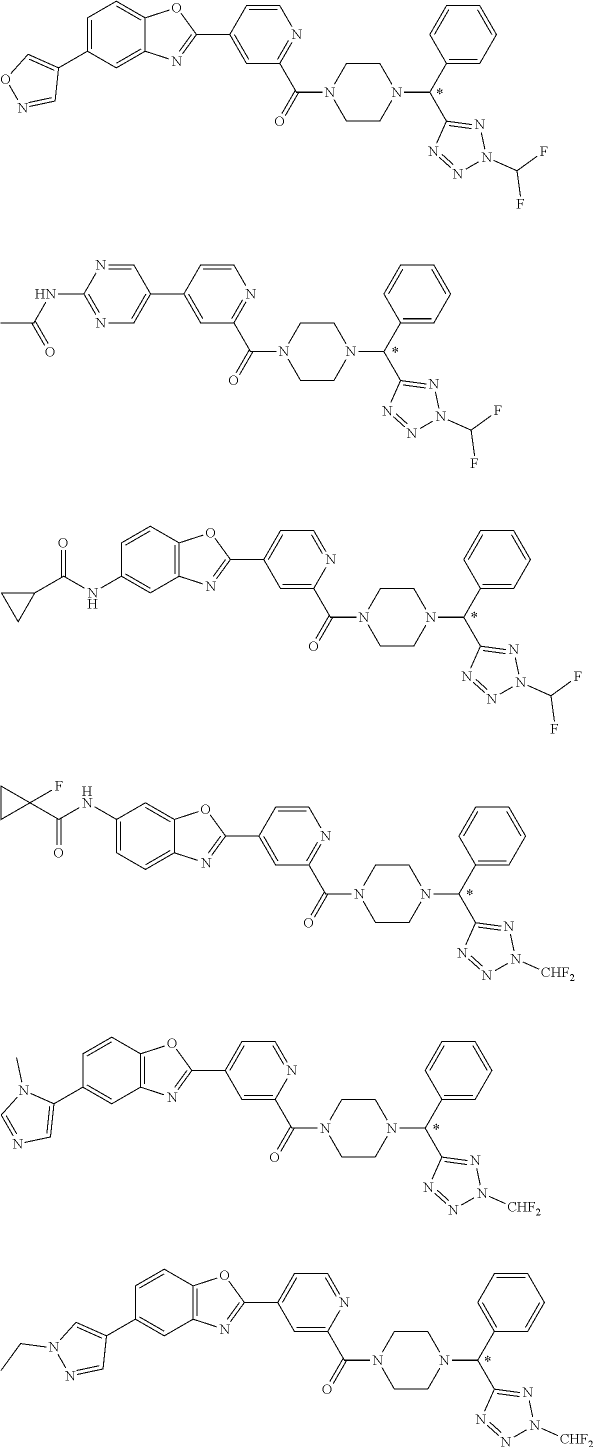 [SMILES: O=C(c1cc(-c2nc3cc(-c4cnoc4)ccc3o2)ccn1)N1CCN(C(c2ccccc2)c2nnn(C(F)F)n2)CC1 ; CC(=O)Nc1ncc(-c2ccnc(C(=O)N3CCN(C(c4ccccc4)c4nnn(C(F)F)n4)CC3)c2)cn1 ; O=C(Nc1ccc2oc(-c3ccnc(C(=O)N4CCN(C(c5ccccc5)c5nnn(C(F)F)n5)CC4)c3)nc2c1)C1CC1 ; O=C(c1cc(-c2nc3ccc(NC(=O)C4(F)CC4)cc3o2)ccn1)N1CCN(C(c2ccccc2)c2nnn(C(F)F)n2)CC1 ; Cn1cncc1-c1ccc2oc(-c3ccnc(C(=O)N4CCN(C(c5ccccc5)c5nnn(C(F)F)n5)CC4)c3)nc2c1 ; CCn1cc(-c2ccc3oc(-c4ccnc(C(=O)N5CCN(C(c6ccccc6)c6nnn(C(F)F)n6)CC5)c4)nc3c2)cn1]

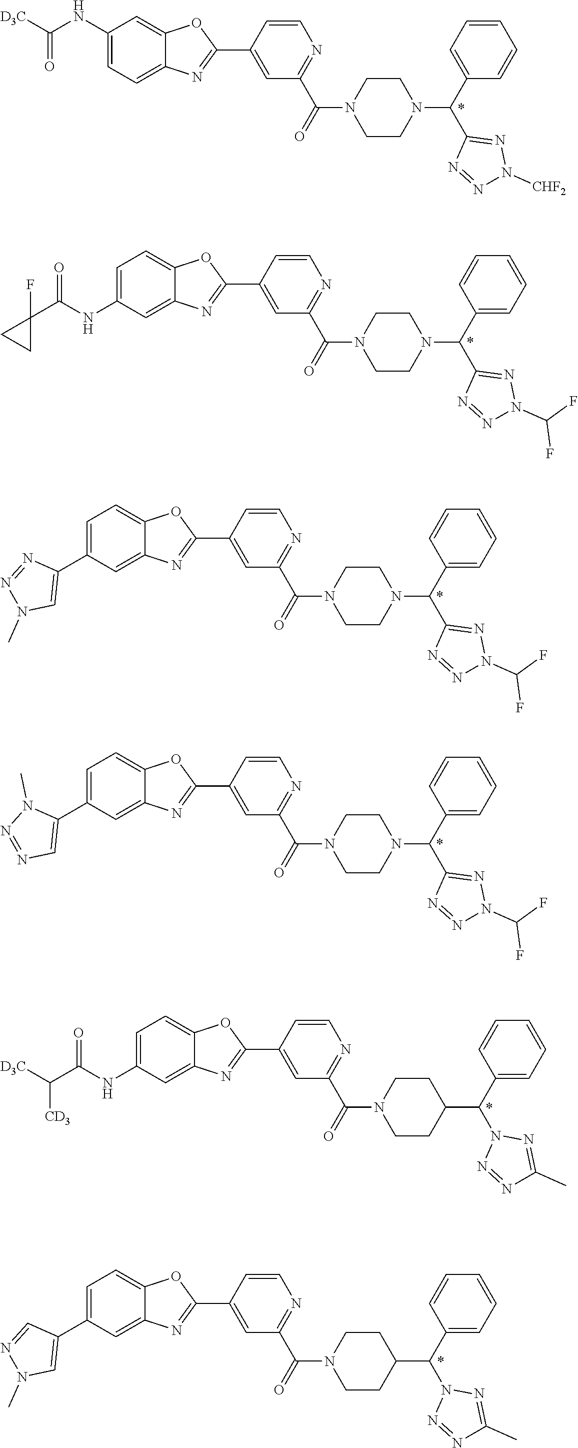

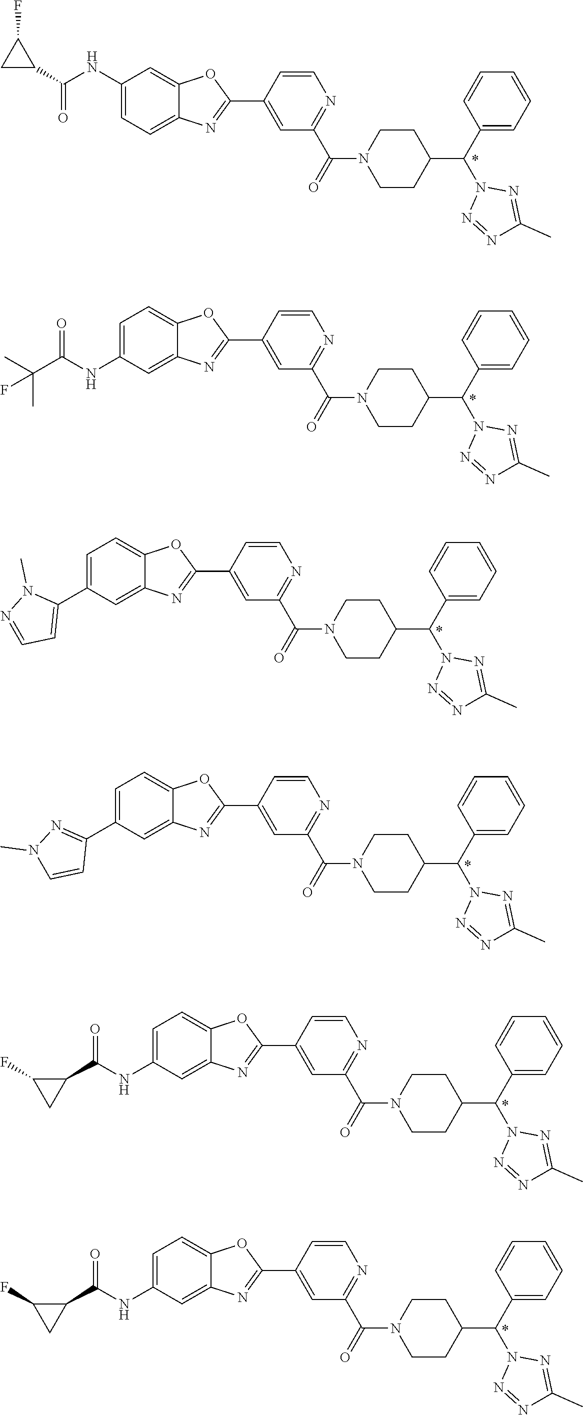

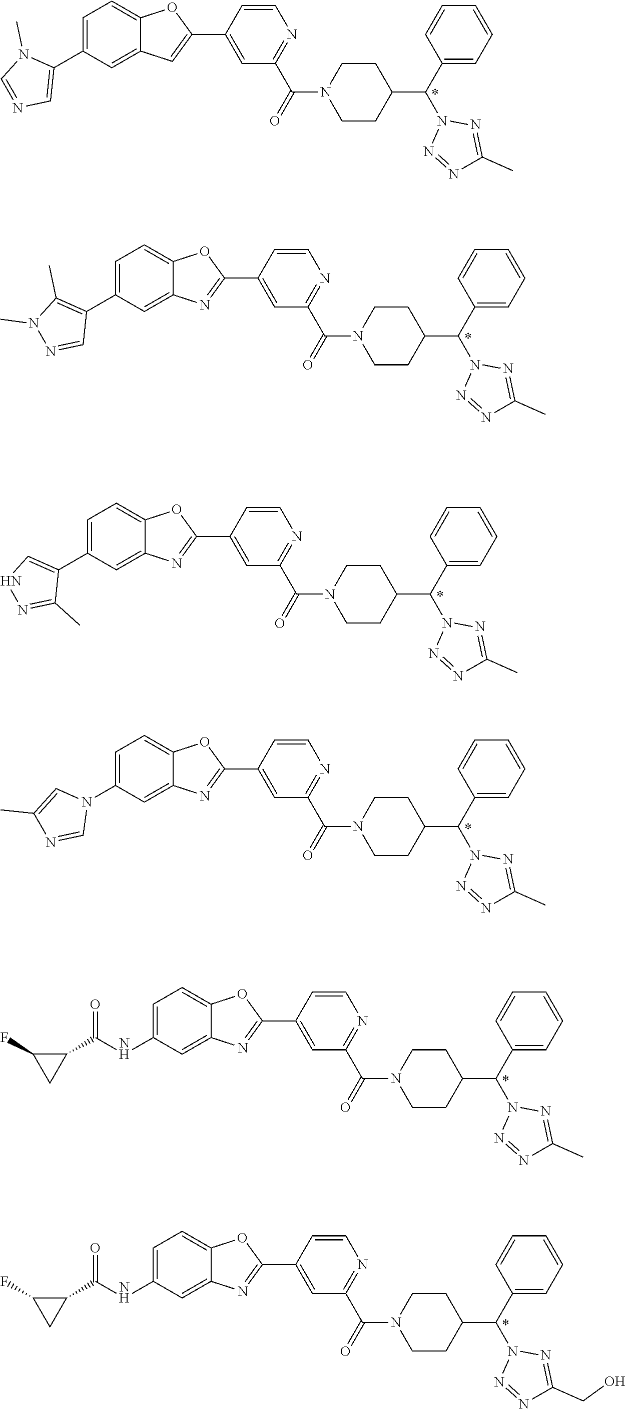

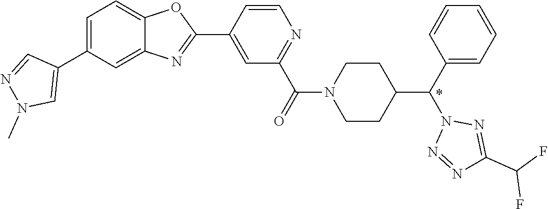 [SMILES: Cn1cc(-c2ccc3oc(-c4ccnc(C(=O)N5CCC(C(c6ccccc6)n6nnc(C(F)F)n6)CC5)c4)nc3c2)cn1]

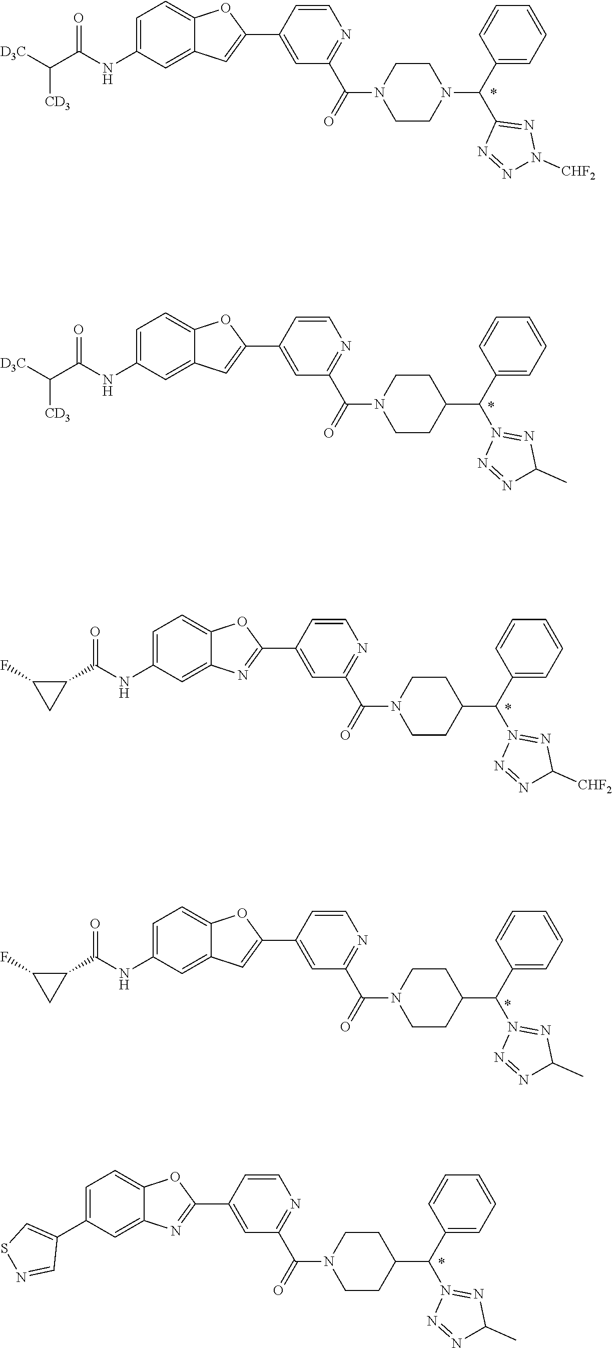

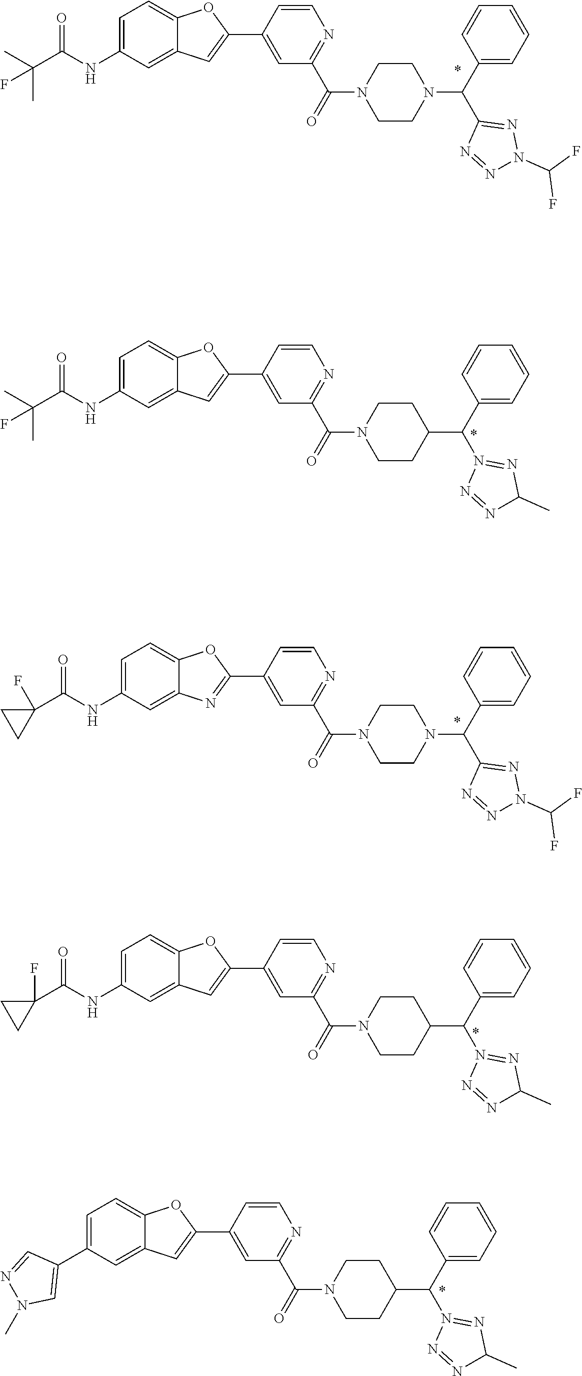

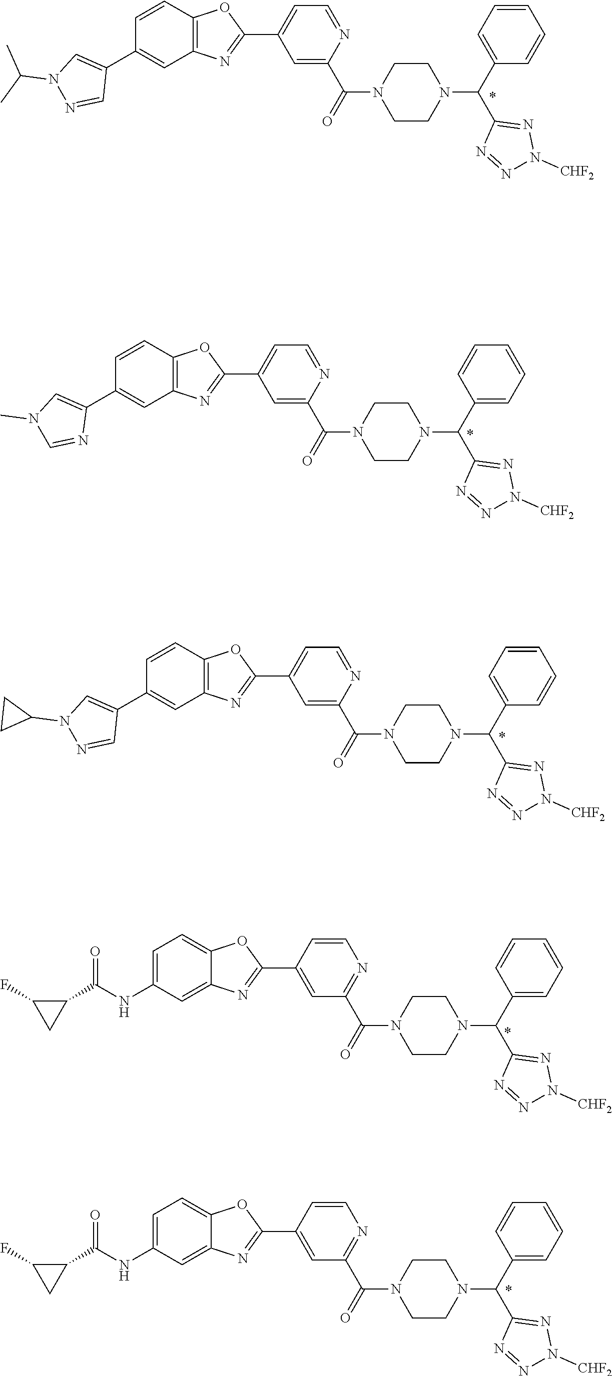 [SMILES: CC(C)n1cc(-c2ccc3oc(-c4ccnc(C(=O)N5CCN(C(c6ccccc6)c6nnn(C(F)F)n6)CC5)c4)nc3c2)cn1 ; Cn1cnc(-c2ccc3oc(-c4ccnc(C(=O)N5CCN(C(c6ccccc6)c6nnn(C(F)F)n6)CC5)c4)nc3c2)c1 ; O=C(c1cc(-c2nc3cc(-c4cnn(C5CC5)c4)ccc3o2)ccn1)N1CCN(C(c2ccccc2)c2nnn(C(F)F)n2)CC1 ; O=C(Nc1ccc2oc(-c3ccnc(C(=O)N4CCN(C(c5ccccc5)c5nnn(C(F)F)n5)CC4)c3)nc2c1)[C@@H]1C[C@@H]1F ; O=C(Nc1ccc2oc(-c3ccnc(C(=O)N4CCN(C(c5ccccc5)c5nnn(C(F)F)n5)CC4)c3)nc2c1)[C@@H]1C[C@@H]1F]

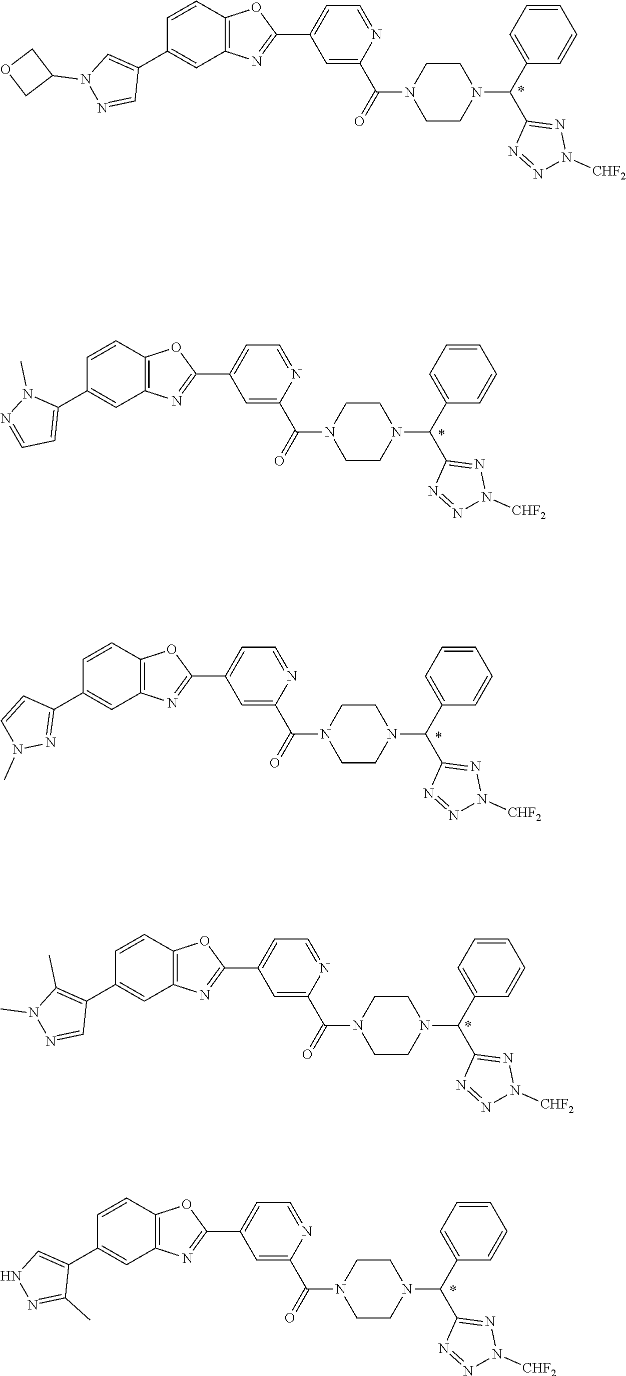 [SMILES: O=C(c1cc(-c2nc3cc(-c4cnn(C5COC5)c4)ccc3o2)ccn1)N1CCN(C(c2ccccc2)c2nnn(C(F)F)n2)CC1 ; Cn1nccc1-c1ccc2oc(-c3ccnc(C(=O)N4CCN(C(c5ccccc5)c5nnn(C(F)F)n5)CC4)c3)nc2c1 ; Cn1ccc(-c2ccc3oc(-c4ccnc(C(=O)N5CCN(C(c6ccccc6)c6nnn(C(F)F)n6)CC5)c4)nc3c2)n1 ; Cc1c(-c2ccc3oc(-c4ccnc(C(=O)N5CCN(C(c6ccccc6)c6nnn(C(F)F)n6)CC5)c4)nc3c2)cnn1C ; Cc1n[nH]cc1-c1ccc2oc(-c3ccnc(C(=O)N4CCN(C(c5ccccc5)c5nnn(C(F)F)n5)CC4)c3)nc2c1]

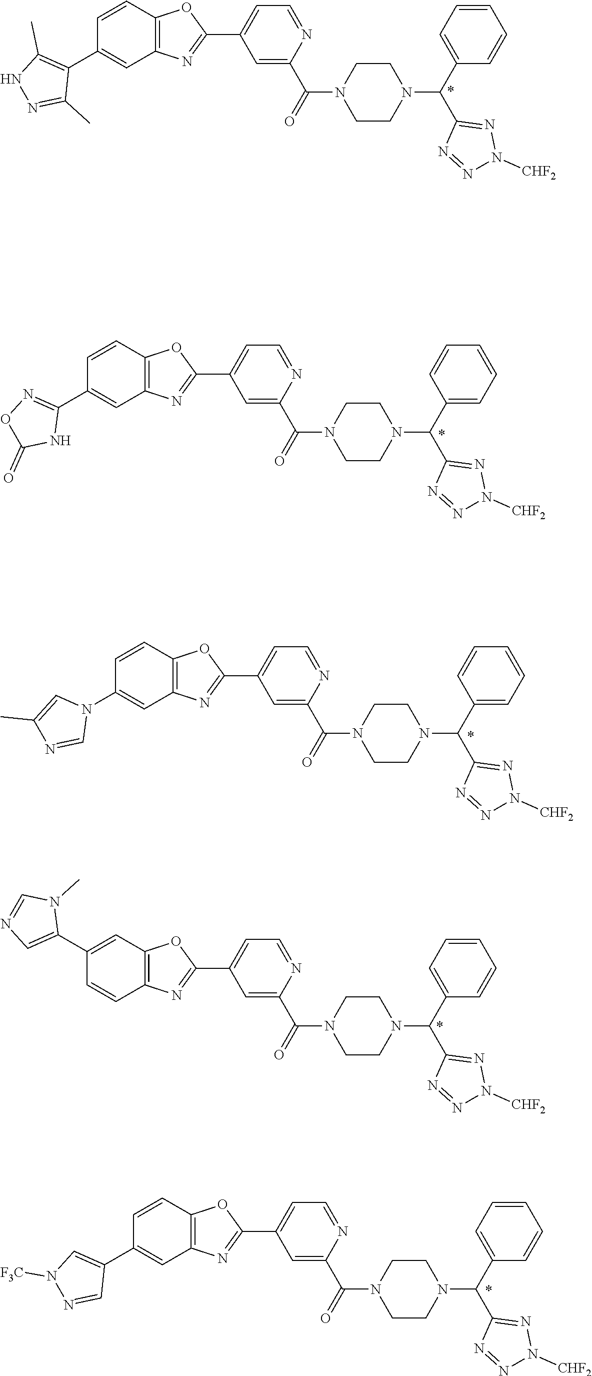 [SMILES: Cc1n[nH]c(C)c1-c1ccc2oc(-c3ccnc(C(=O)N4CCN(C(c5ccccc5)c5nnn(C(F)F)n5)CC4)c3)nc2c1 ; O=C(c1cc(-c2nc3cc(-c4noc(=O)[nH]4)ccc3o2)ccn1)N1CCN(C(c2ccccc2)c2nnn(C(F)F)n2)CC1 ; Cc1cn(-c2ccc3oc(-c4ccnc(C(=O)N5CCN(C(c6ccccc6)c6nnn(C(F)F)n6)CC5)c4)nc3c2)cn1 ; Cn1cncc1-c1ccc2nc(-c3ccnc(C(=O)N4CCN(C(c5ccccc5)c5nnn(C(F)F)n5)CC4)c3)oc2c1 ; O=C(c1cc(-c2nc3cc(-c4cnn(C(F)(F)F)c4)ccc3o2)ccn1)N1CCN(C(c2ccccc2)c2nnn(C(F)F)n2)CC1]

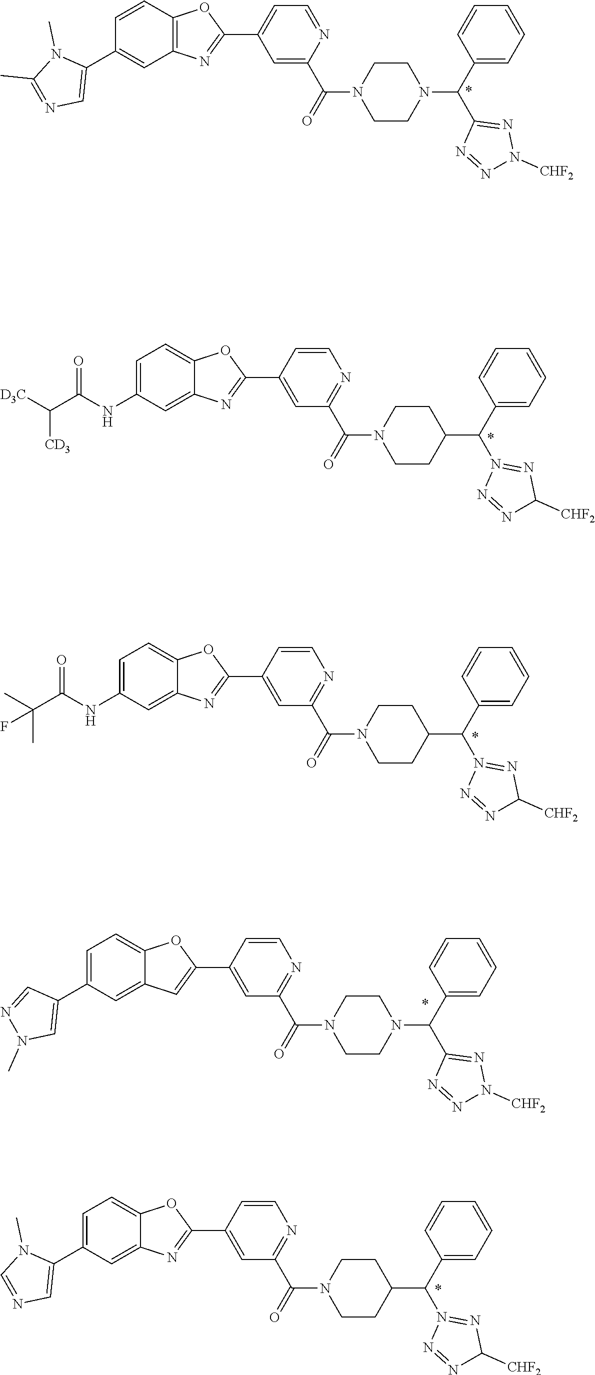

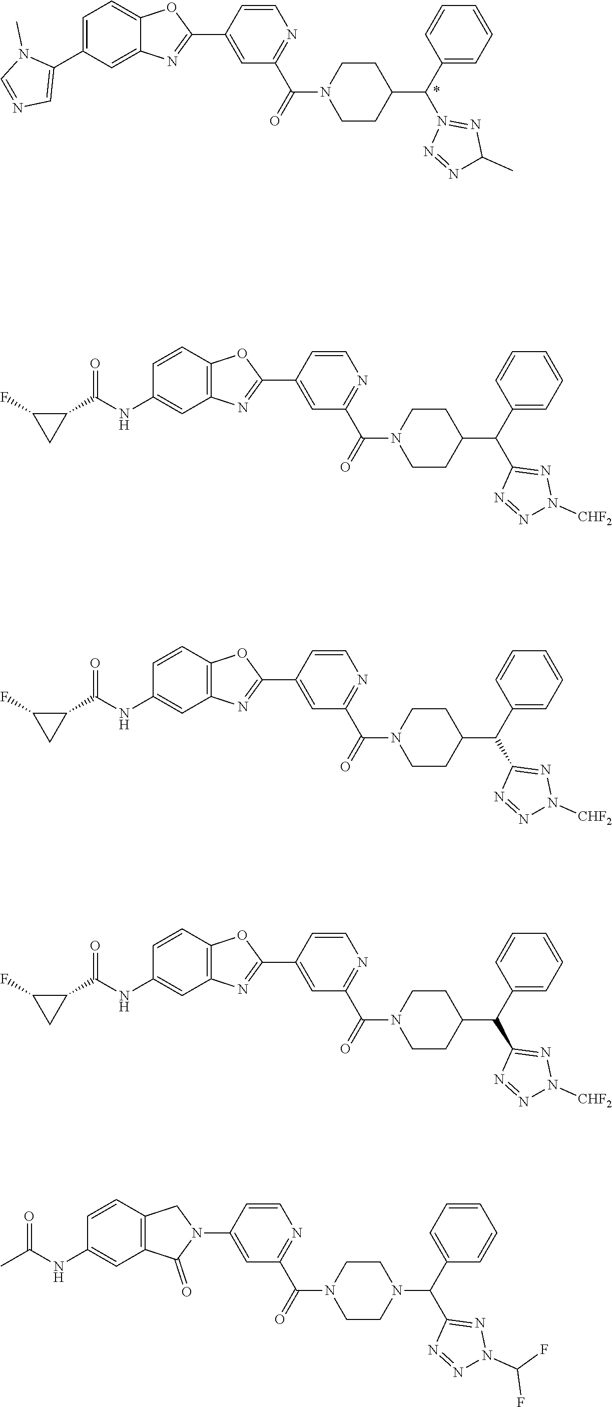

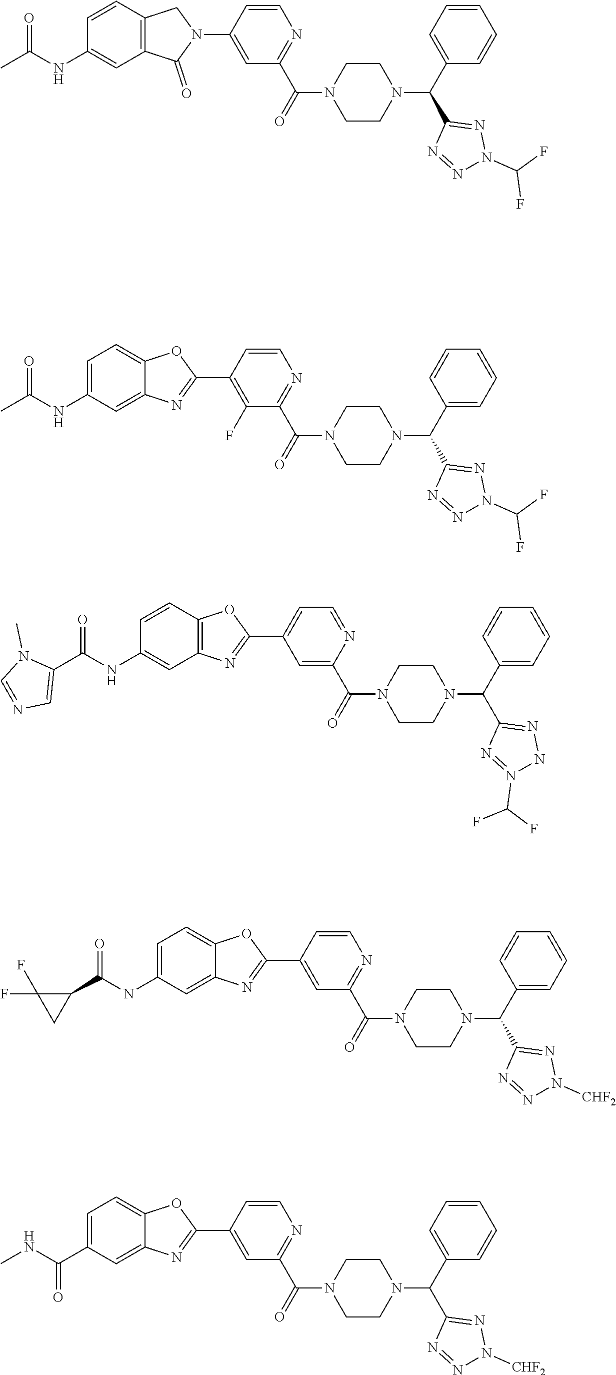

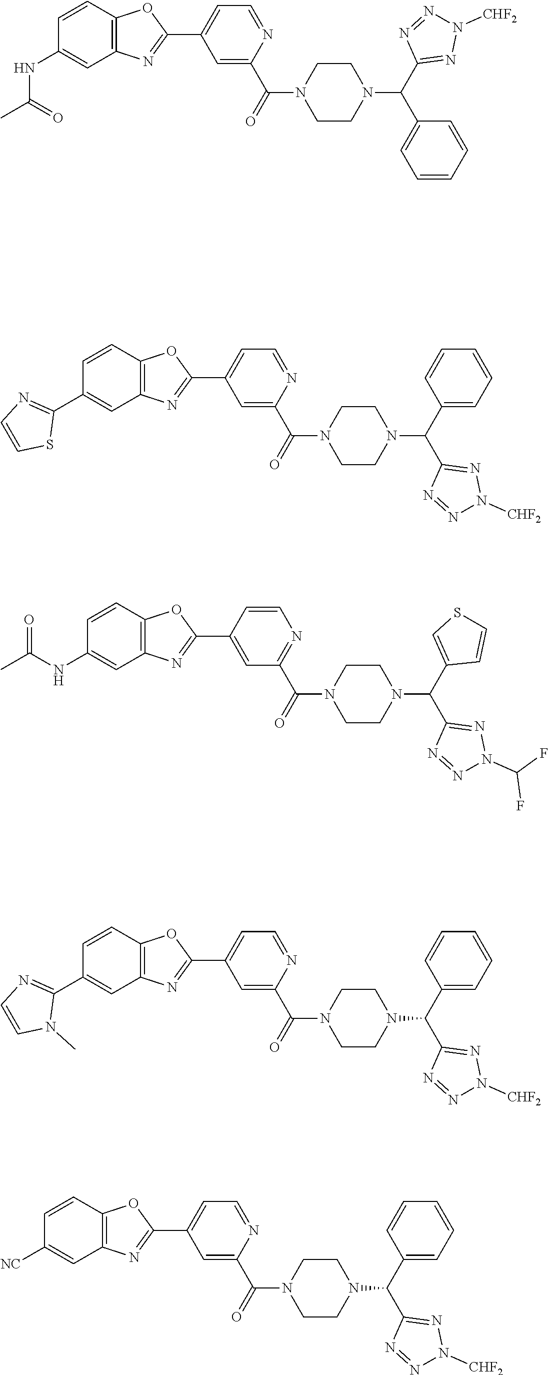 [SMILES: CC(=O)Nc1ccc2oc(-c3ccnc(C(=O)N4CCN(C(c5ccccc5)c5nnn(C(F)F)n5)CC4)c3)nc2c1 ; O=C(c1cc(-c2nc3cc(-c4nccs4)ccc3o2)ccn1)N1CCN(C(c2ccccc2)c2nnn(C(F)F)n2)CC1 ; CC(=O)Nc1ccc2oc(-c3ccnc(C(=O)N4CCN(C(c5ccsc5)c5nnn(C(F)F)n5)CC4)c3)nc2c1 ; Cn1ccnc1-c1ccc2oc(-c3ccnc(C(=O)N4CCN([C@H](c5ccccc5)c5nnn(C(F)F)n5)CC4)c3)nc2c1 ; N#Cc1ccc2oc(-c3ccnc(C(=O)N4CCN([C@H](c5ccccc5)c5nnn(C(F)F)n5)CC4)c3)nc2c1]

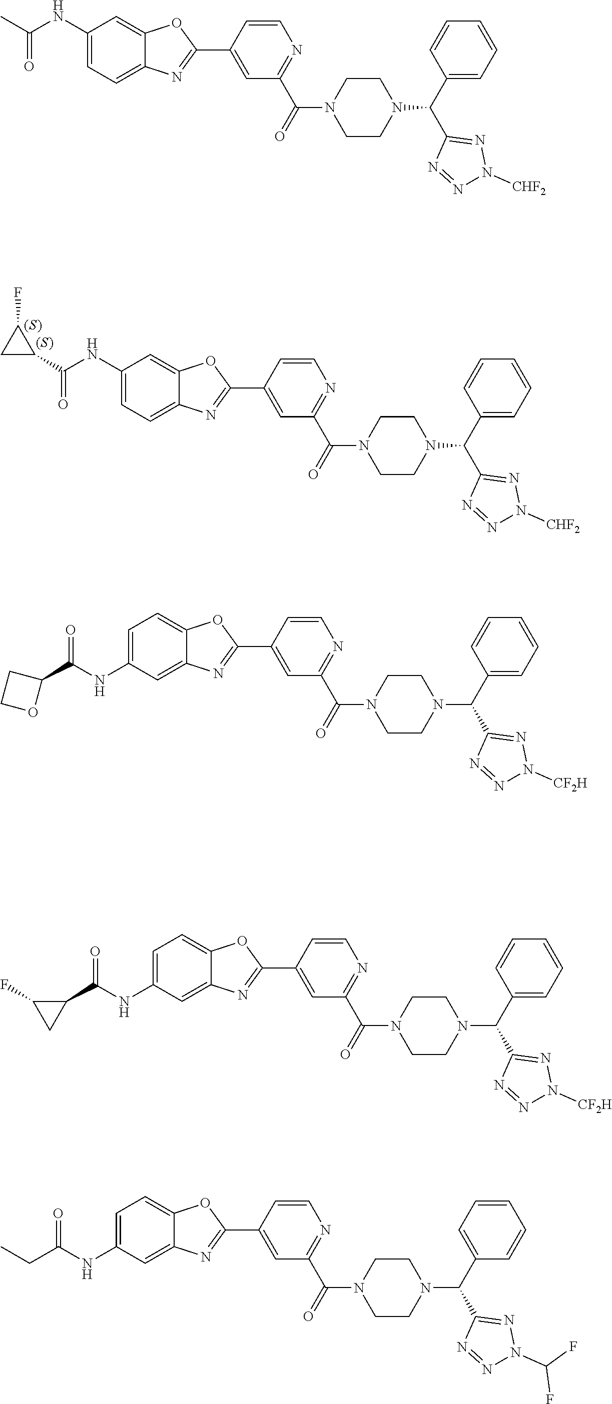

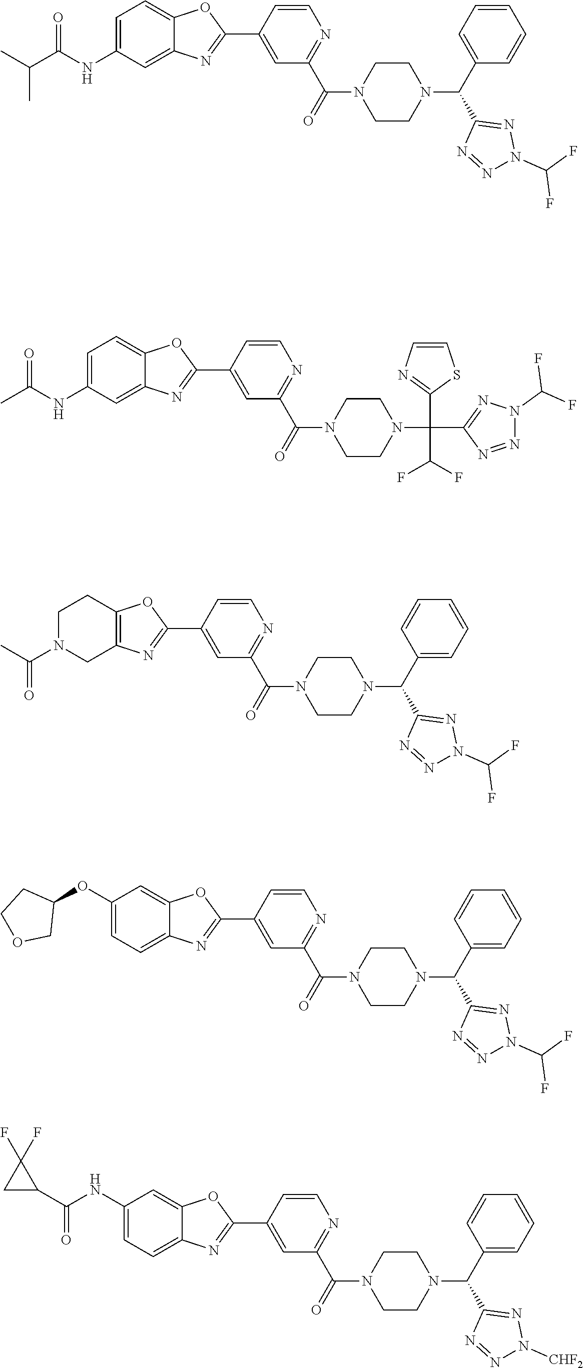 [SMILES: CC(C)C(=O)Nc1ccc2oc(-c3ccnc(C(=O)N4CCN([C@H](c5ccccc5)c5nnn(C(F)F)n5)CC4)c3)nc2c1 ; CC(=O)Nc1ccc2oc(-c3ccnc(C(=O)N4CCN(C(c5nnn(C(F)F)n5)(c5nccs5)C(F)F)CC4)c3)nc2c1 ; CC(=O)N1CCc2oc(-c3ccnc(C(=O)N4CCN([C@H](c5ccccc5)c5nnn(C(F)F)n5)CC4)c3)nc2C1 ; O=C(c1cc(-c2nc3ccc(O[C@@H]4CCOC4)cc3o2)ccn1)N1CCN([C@H](c2ccccc2)c2nnn(C(F)F)n2)CC1 ; O=C(Nc1ccc2nc(-c3ccnc(C(=O)N4CCN([C@H](c5ccccc5)c5nnn(C(F)F)n5)CC4)c3)oc2c1)C1CC1(F)F]

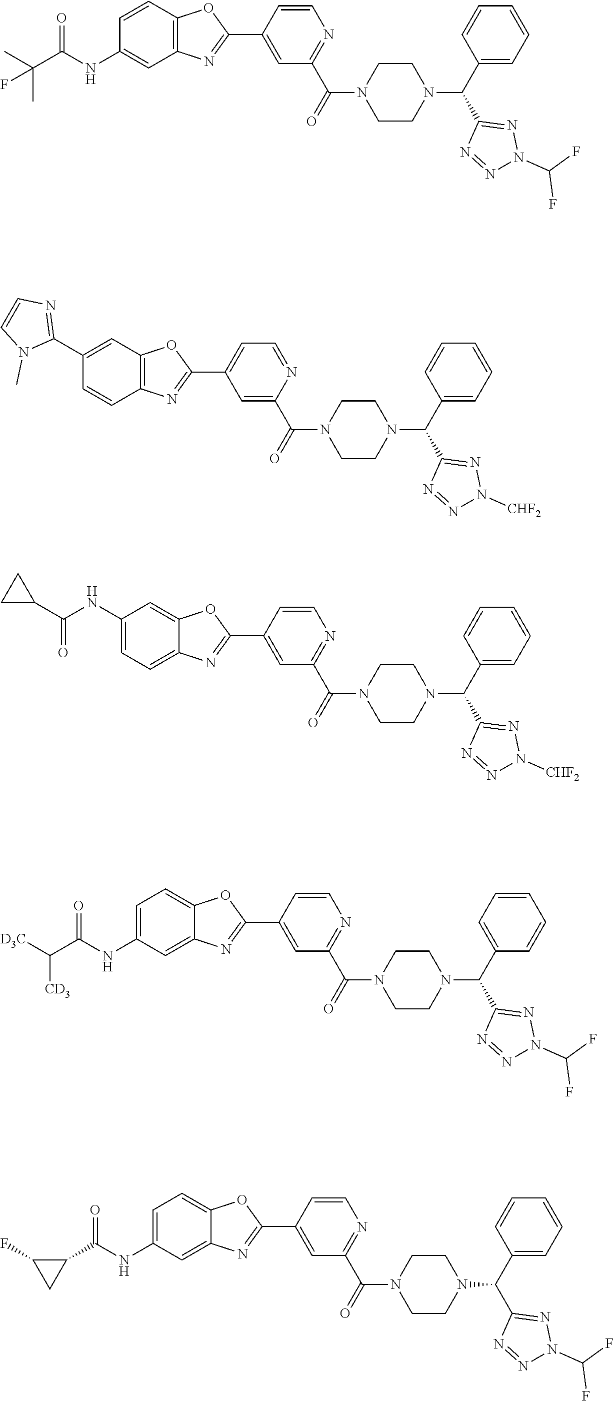 [SMILES: CC(C)(F)C(=O)Nc1ccc2oc(-c3ccnc(C(=O)N4CCN([C@H](c5ccccc5)c5nnn(C(F)F)n5)CC4)c3)nc2c1 ; Cn1ccnc1-c1ccc2nc(-c3ccnc(C(=O)N4CCN([C@H](c5ccccc5)c5nnn(C(F)F)n5)CC4)c3)oc2c1 ; O=C(Nc1ccc2nc(-c3ccnc(C(=O)N4CCN([C@H](c5ccccc5)c5nnn(C(F)F)n5)CC4)c3)oc2c1)C1CC1 ; [2H]C([2H])([2H])C(C(=O)Nc1ccc2oc(-c3ccnc(C(=O)N4CCN([C@H](c5ccccc5)c5nnn(C(F)F)n5)CC4)c3)nc2c1)C([2H])([2H])[2H] ; O=C(Nc1ccc2oc(-c3ccnc(C(=O)N4CCN([C@H](c5ccccc5)c5nnn(C(F)F)n5)CC4)c3)nc2c1)[C@@H]1C[C@@H]1F]

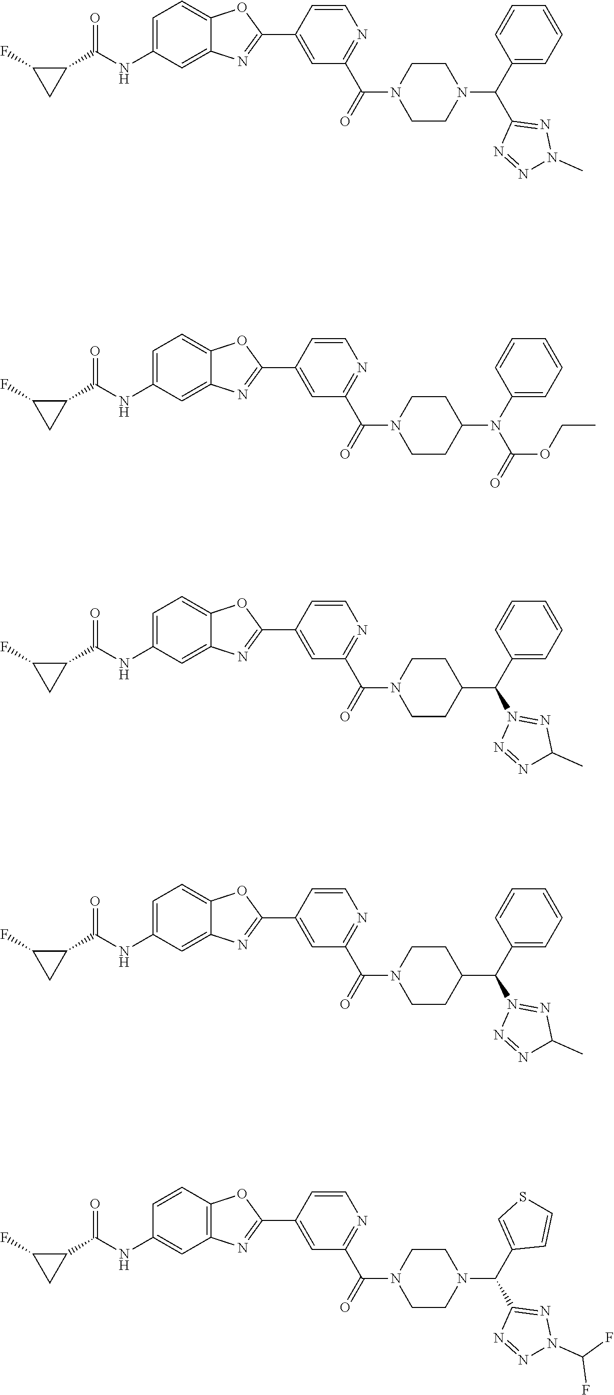

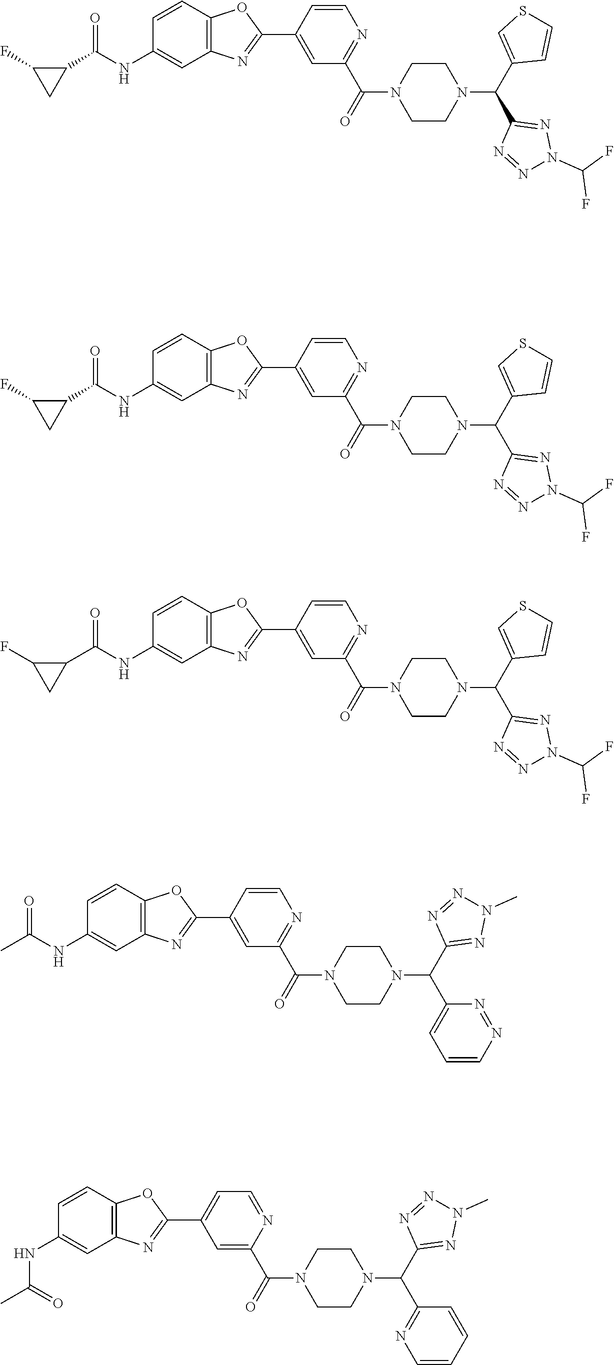

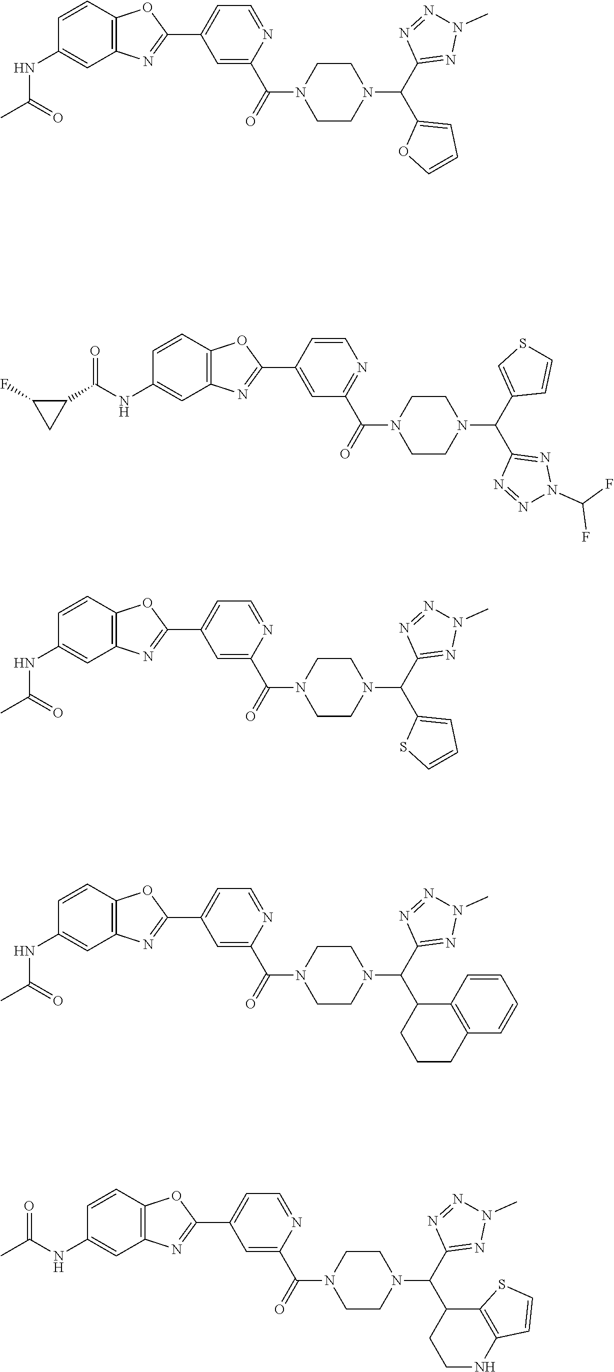

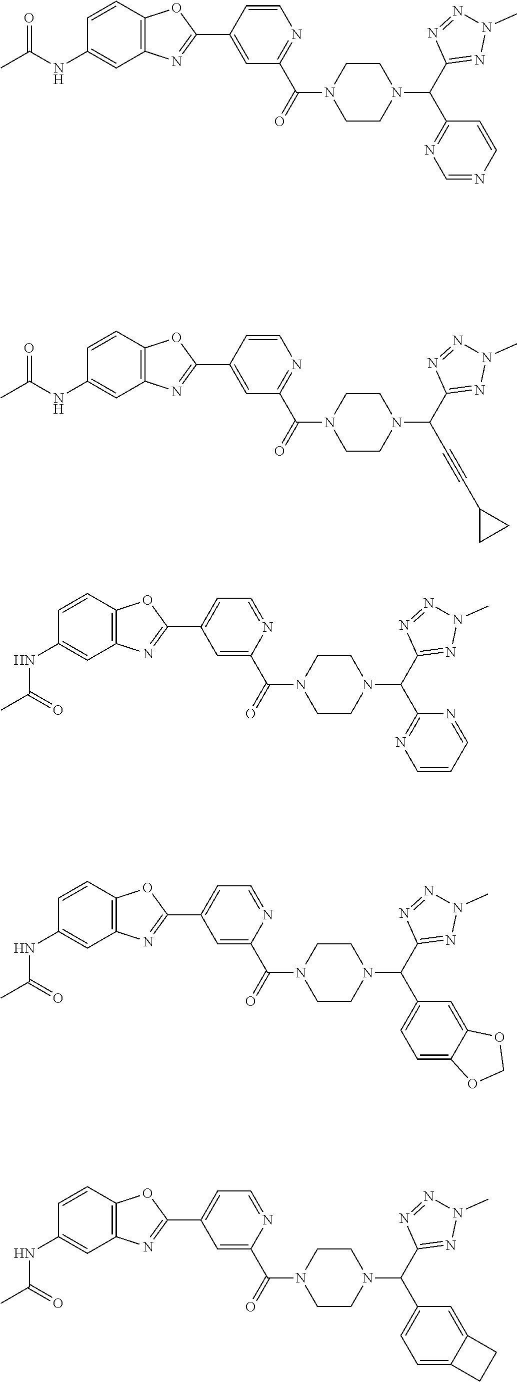

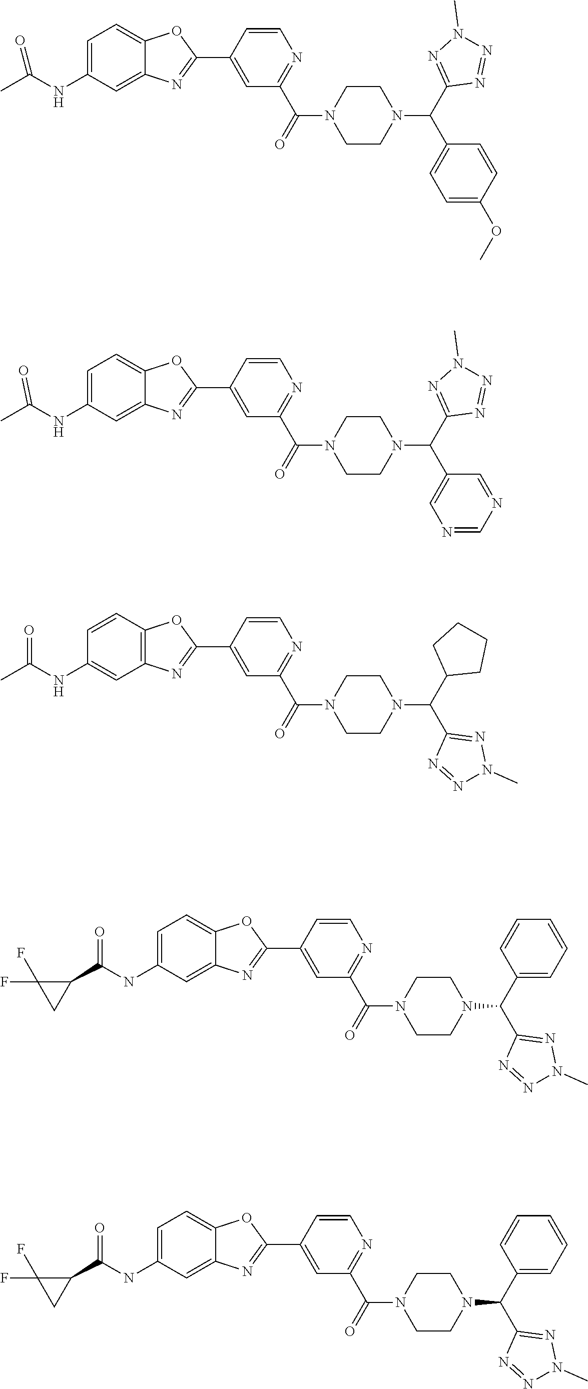 [SMILES: COc1ccc(C(c2nnn(C)n2)N2CCN(C(=O)c3cc(-c4nc5cc(NC(C)=O)ccc5o4)ccn3)CC2)cc1 ; CC(=O)Nc1ccc2oc(-c3ccnc(C(=O)N4CCN(C(c5cncnc5)c5nnn(C)n5)CC4)c3)nc2c1 ; CC(=O)Nc1ccc2oc(-c3ccnc(C(=O)N4CCN(C(c5nnn(C)n5)C5CCCC5)CC4)c3)nc2c1 ; Cn1nnc([C@@H](c2ccccc2)N2CCN(C(=O)c3cc(-c4nc5cc(NC(=O)[C@H]6CC6(F)F)ccc5o4)ccn3)CC2)n1 ; Cn1nnc([C@H](c2ccccc2)N2CCN(C(=O)c3cc(-c4nc5cc(NC(=O)[C@H]6CC6(F)F)ccc5o4)ccn3)CC2)n1]

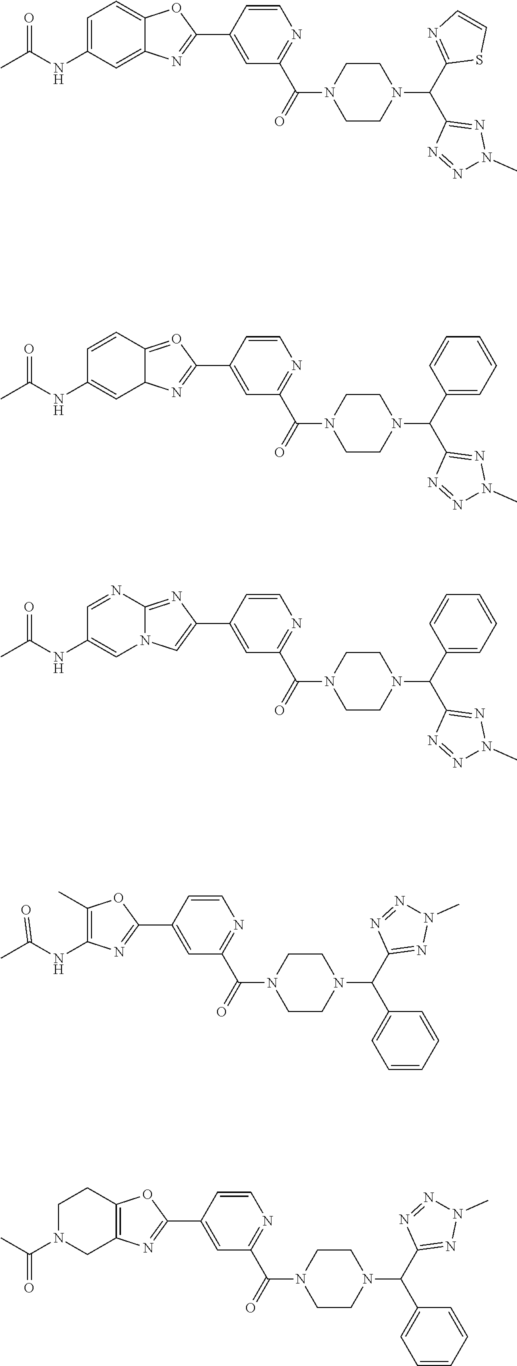

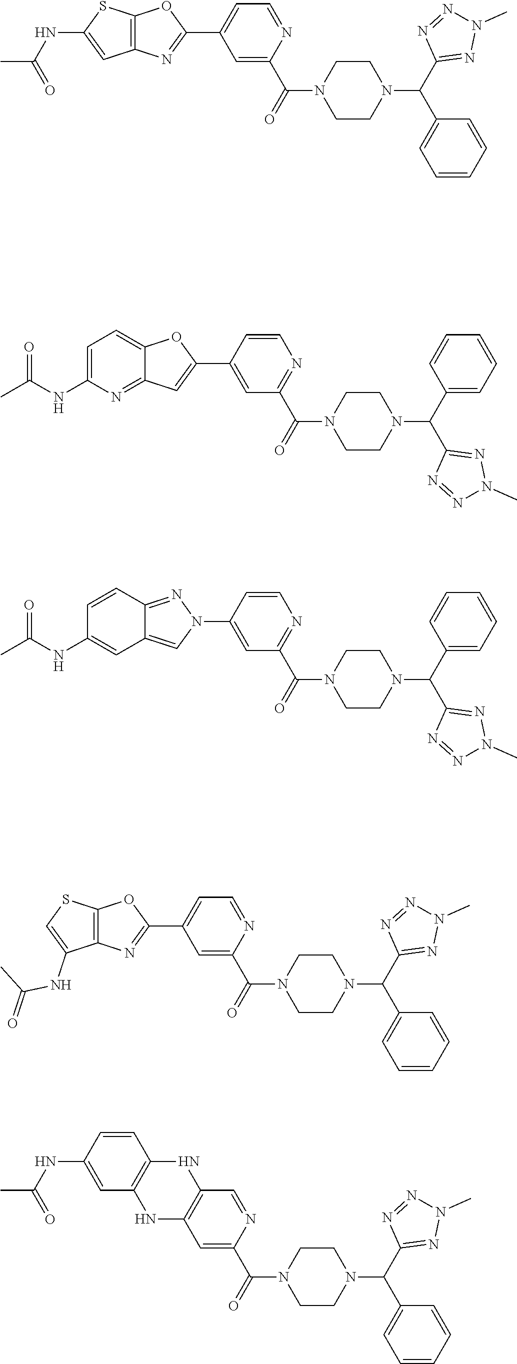

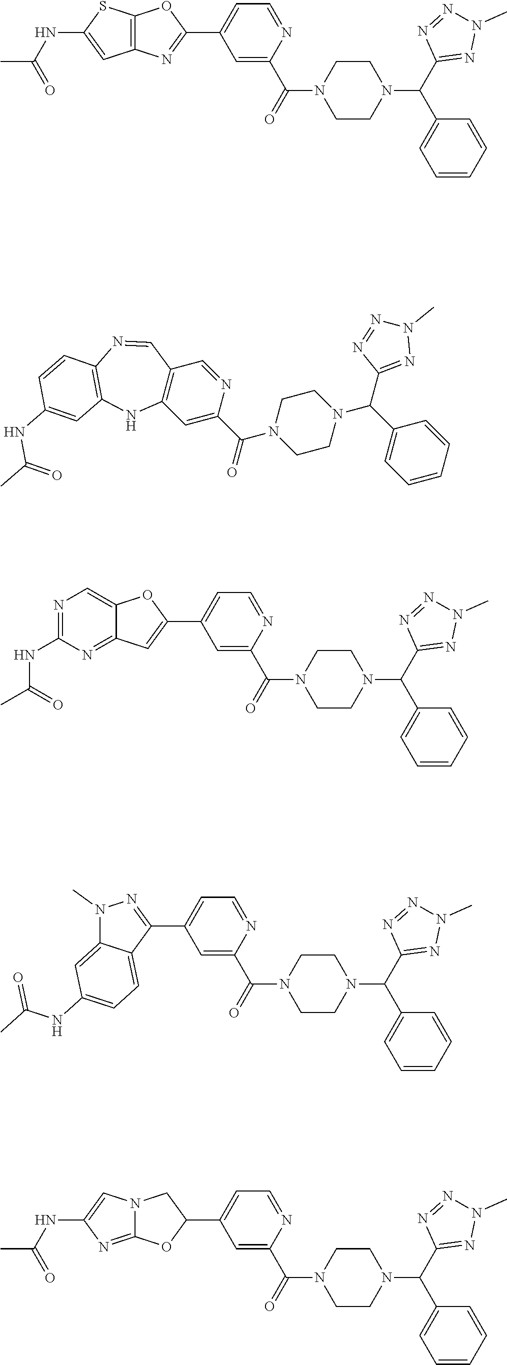

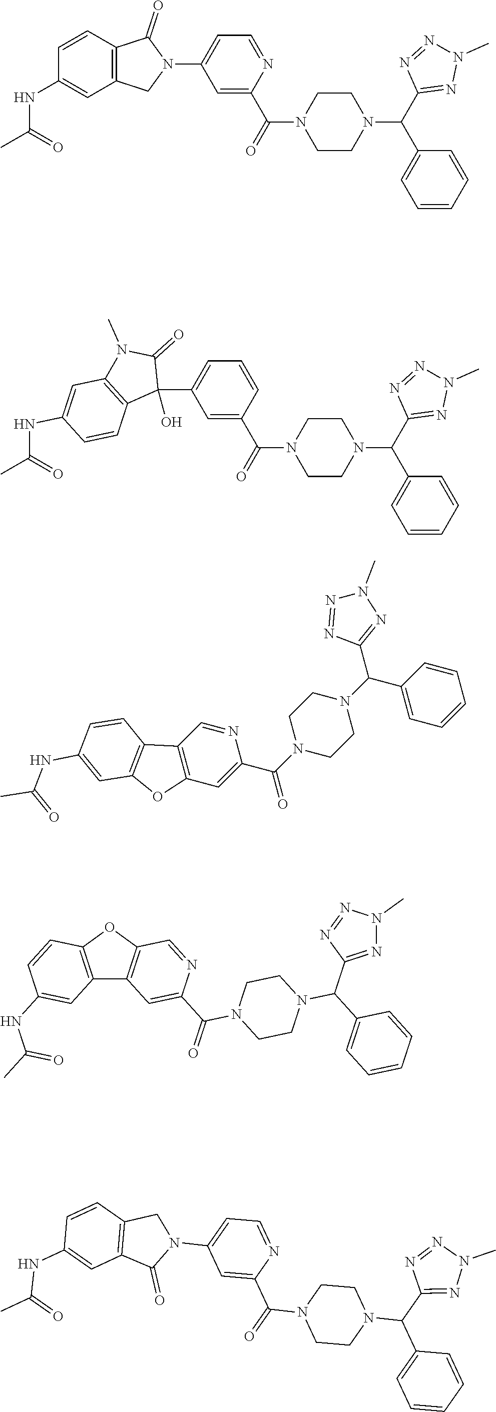

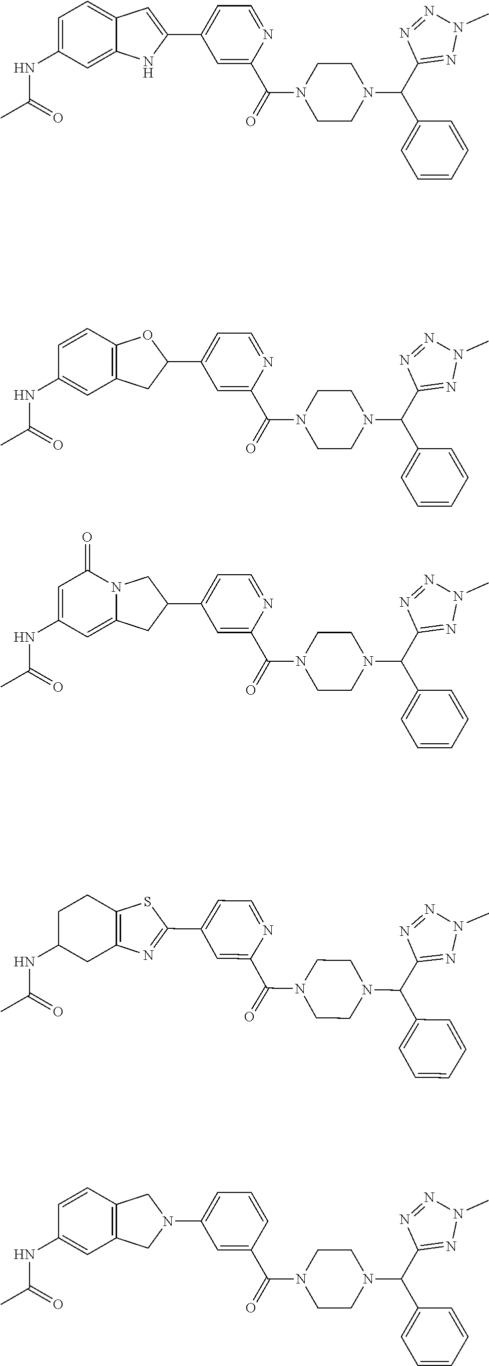

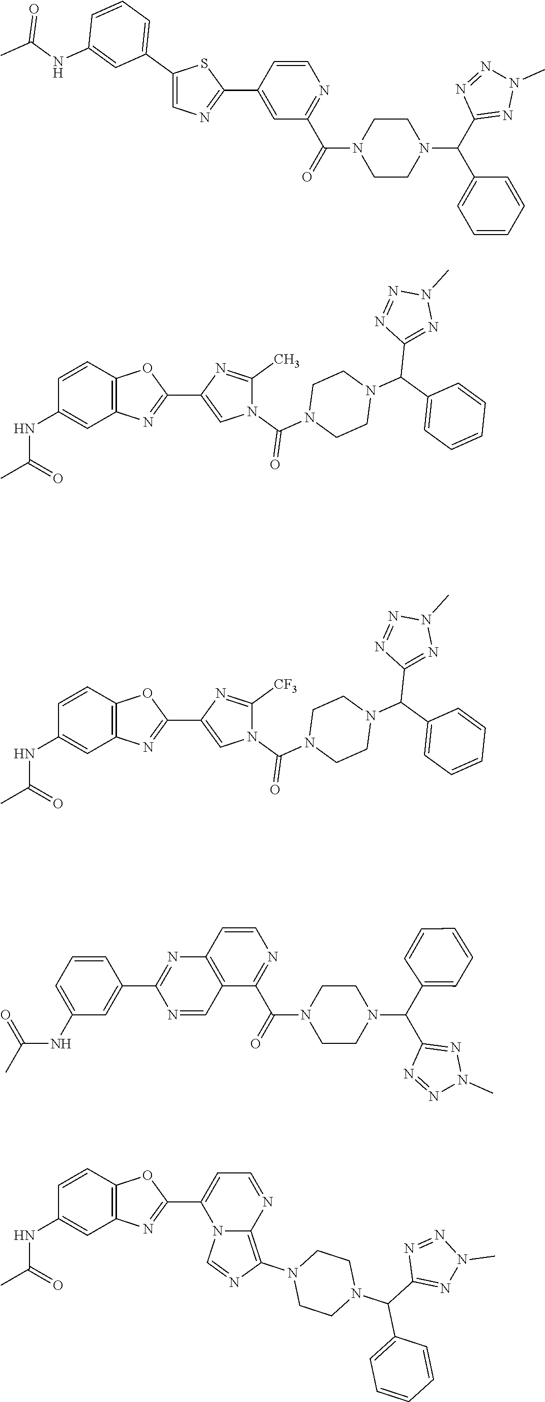

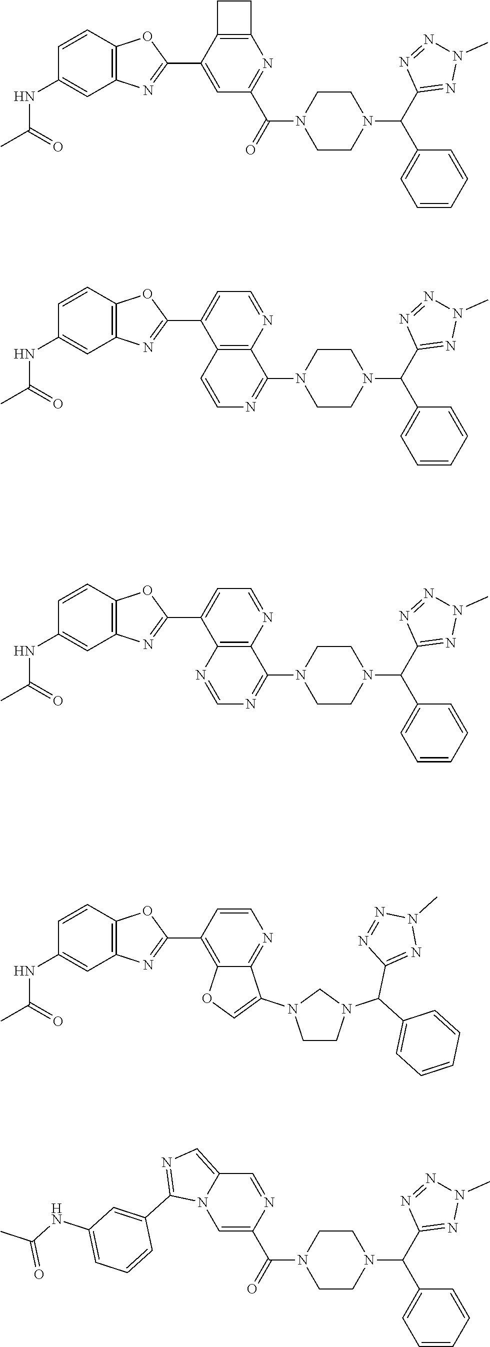

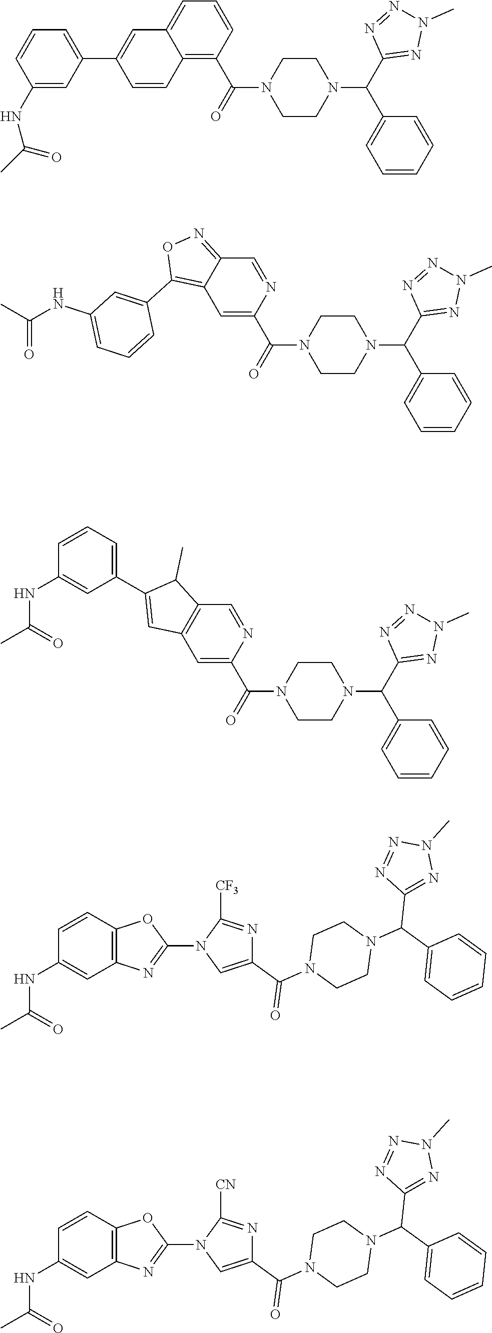

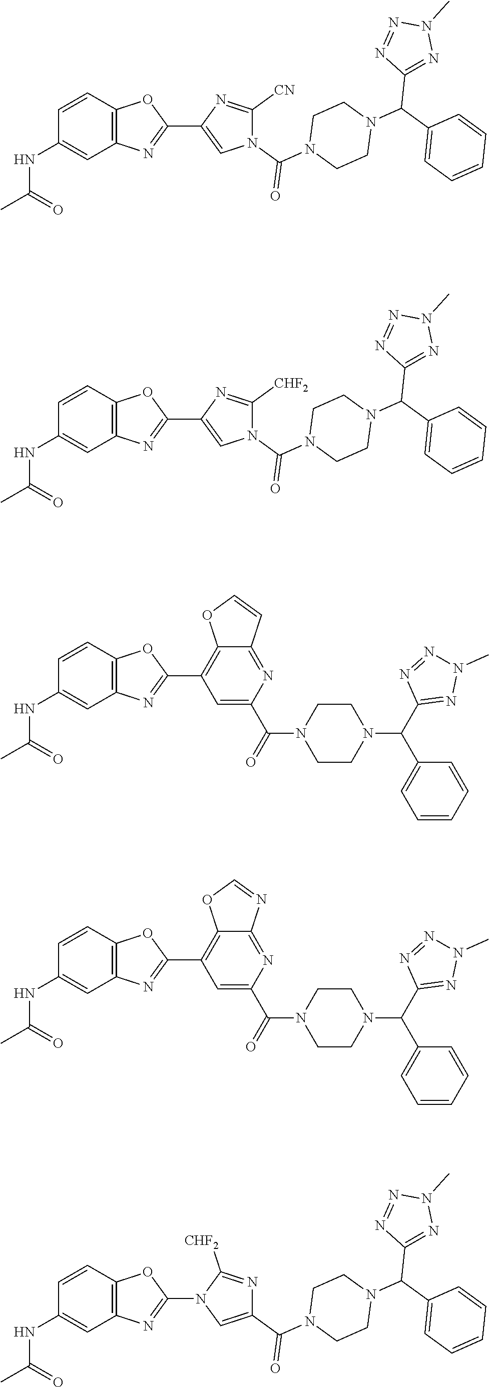

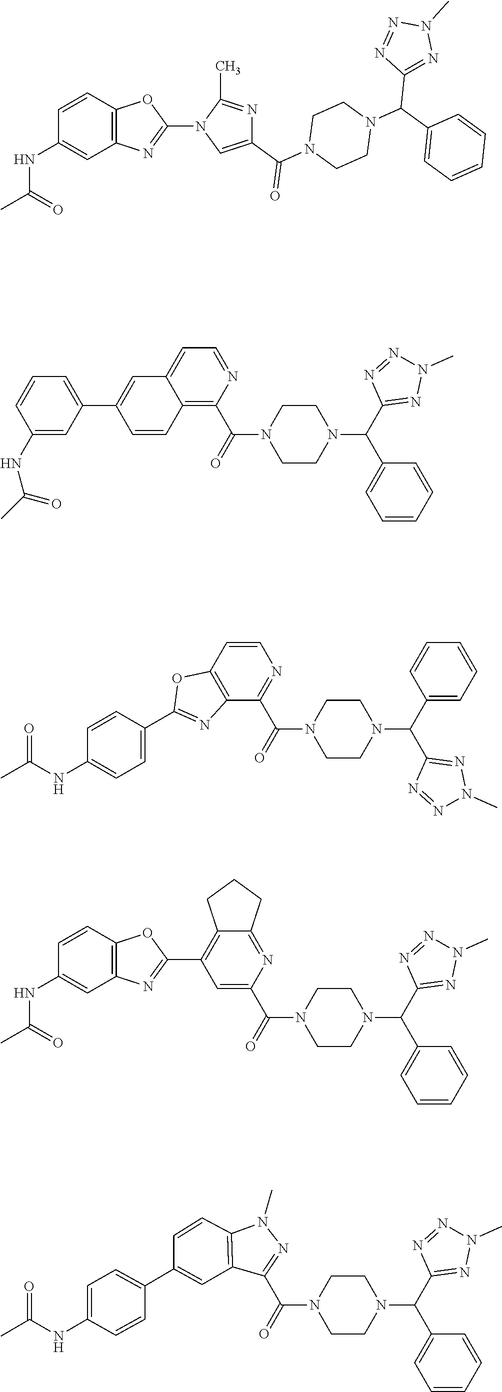

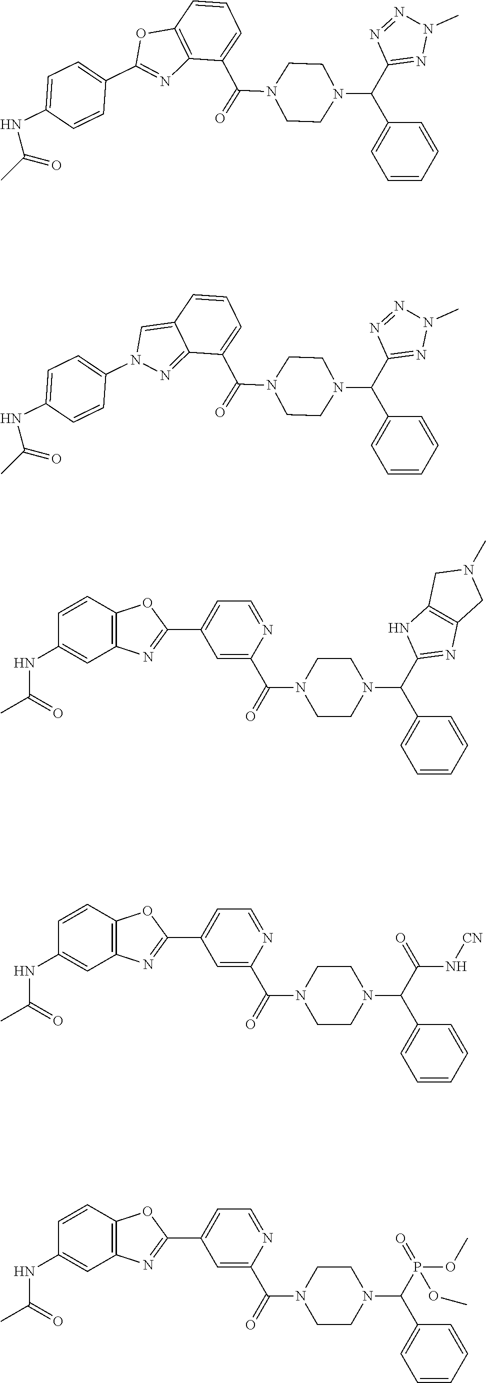

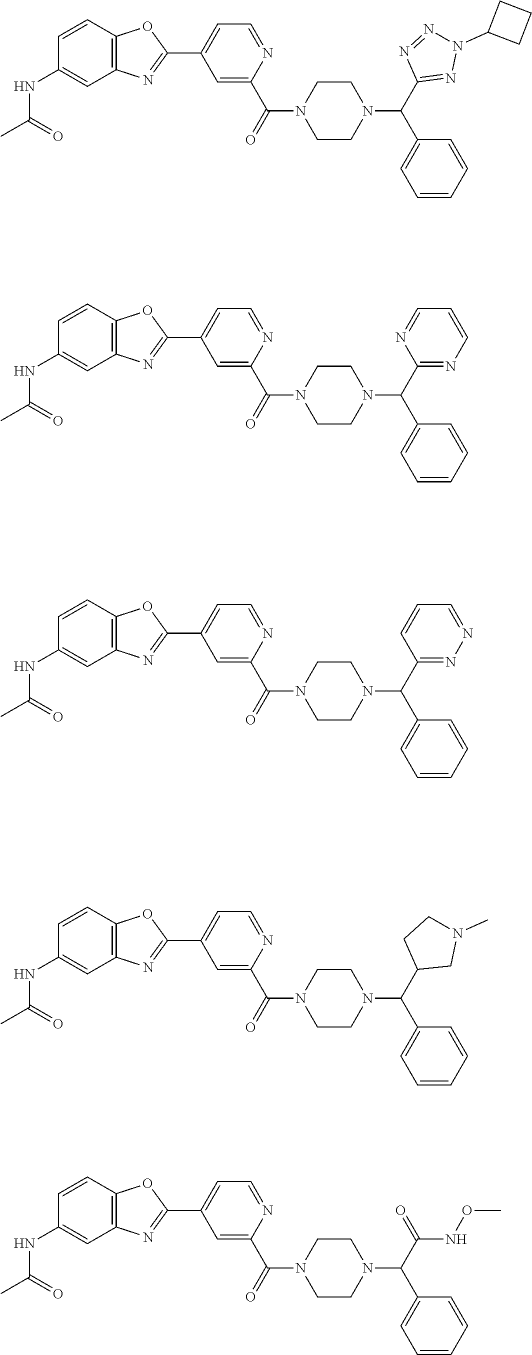

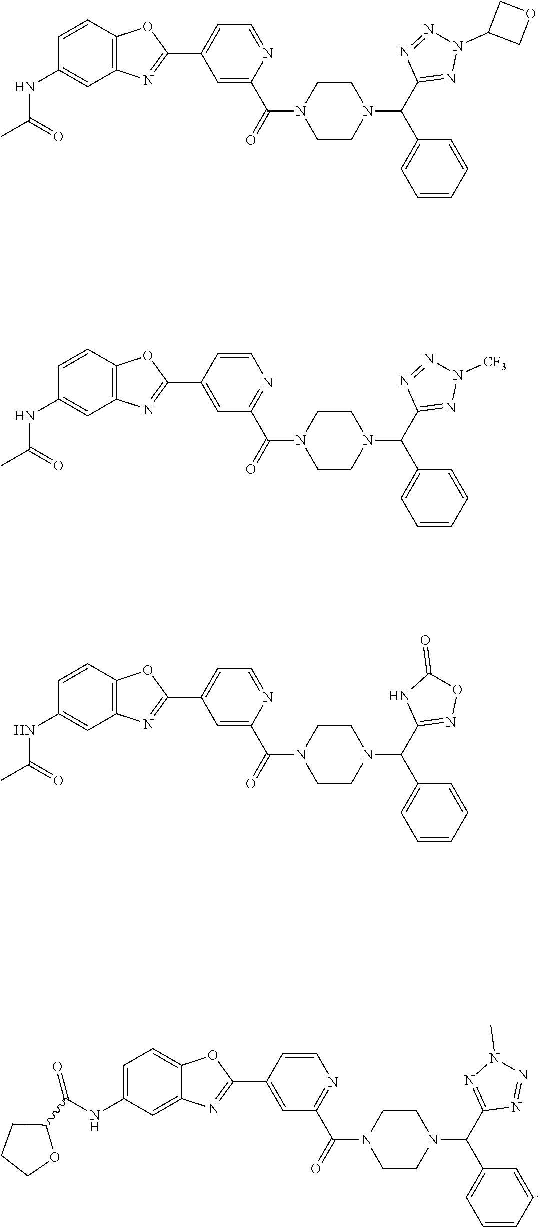 [SMILES: CC(=O)Nc1ccc2oc(-c3ccnc(C(=O)N4CCN(C(c5ccccc5)c5nnn(C6COC6)n5)CC4)c3)nc2c1 ; CC(=O)Nc1ccc2oc(-c3ccnc(C(=O)N4CCN(C(c5ccccc5)c5nnn(C(F)(F)F)n5)CC4)c3)nc2c1 ; CC(=O)Nc1ccc2oc(-c3ccnc(C(=O)N4CCN(C(c5ccccc5)c5noc(=O)[nH]5)CC4)c3)nc2c1 ; Cn1nnc(C(c2ccccc2)N2CCN(C(=O)c3cc(-c4nc5cc(NC(=O)C6CCCO6)ccc5o4)ccn3)CC2)n1]

The compound of the present disclosure can be selected from the following structures, the isomer thereof or the pharmaceutically acceptable salt thereof:

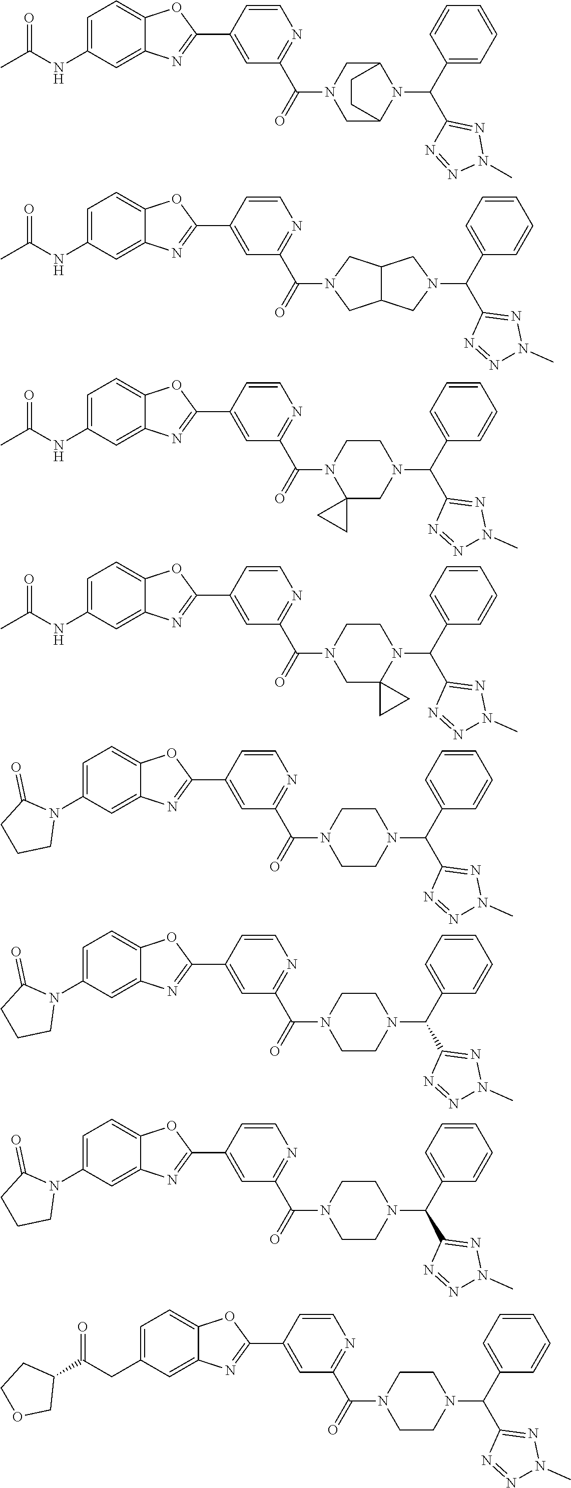

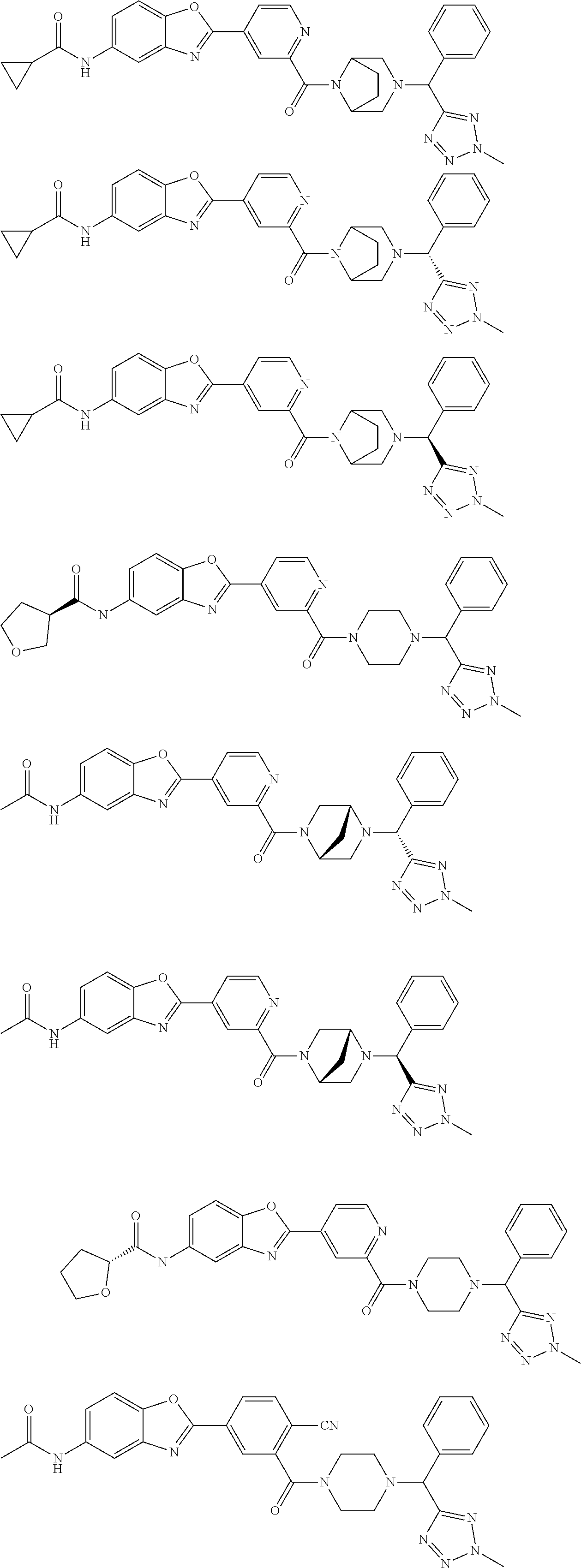

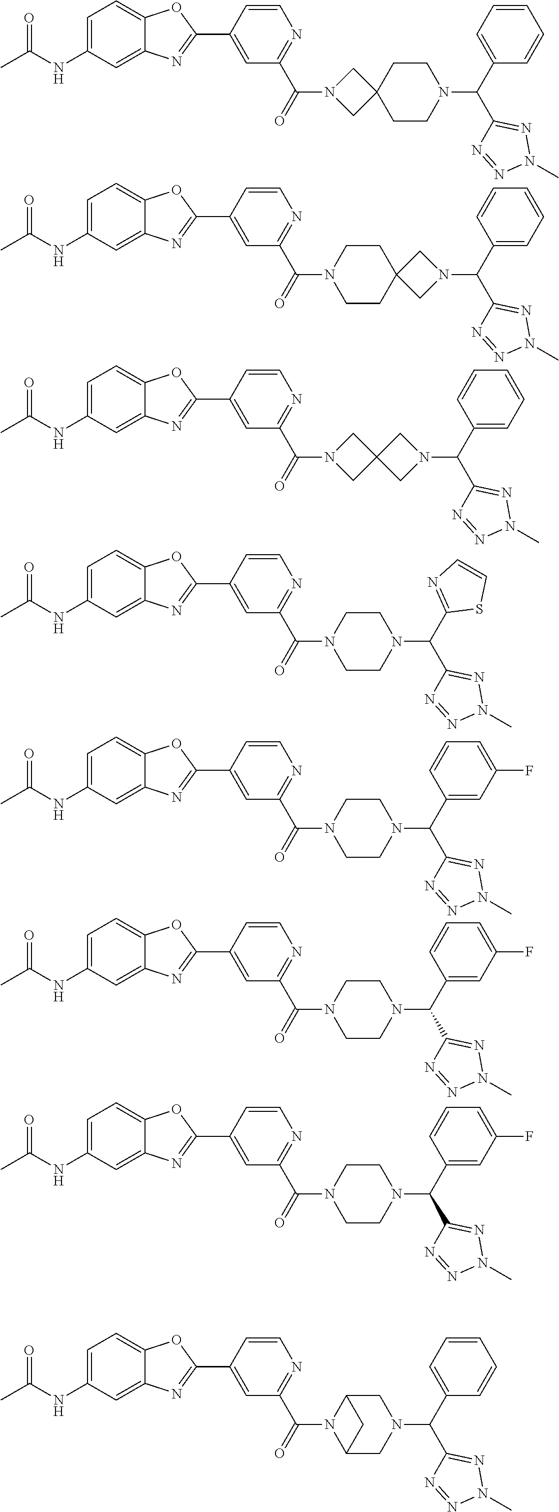

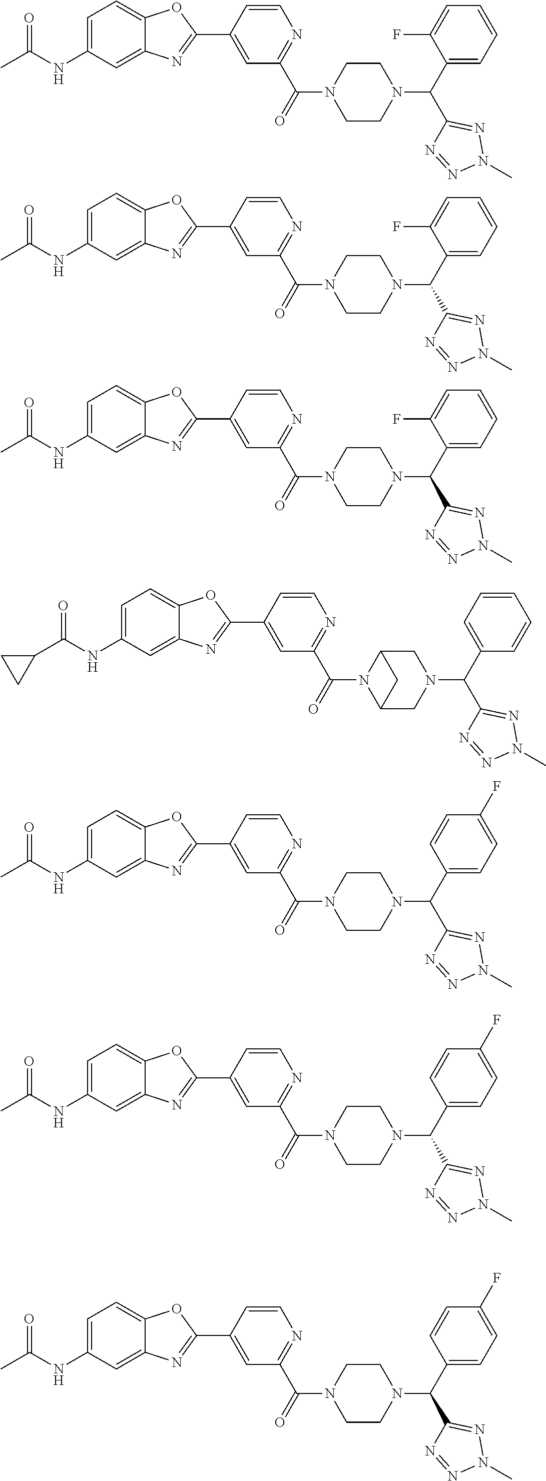

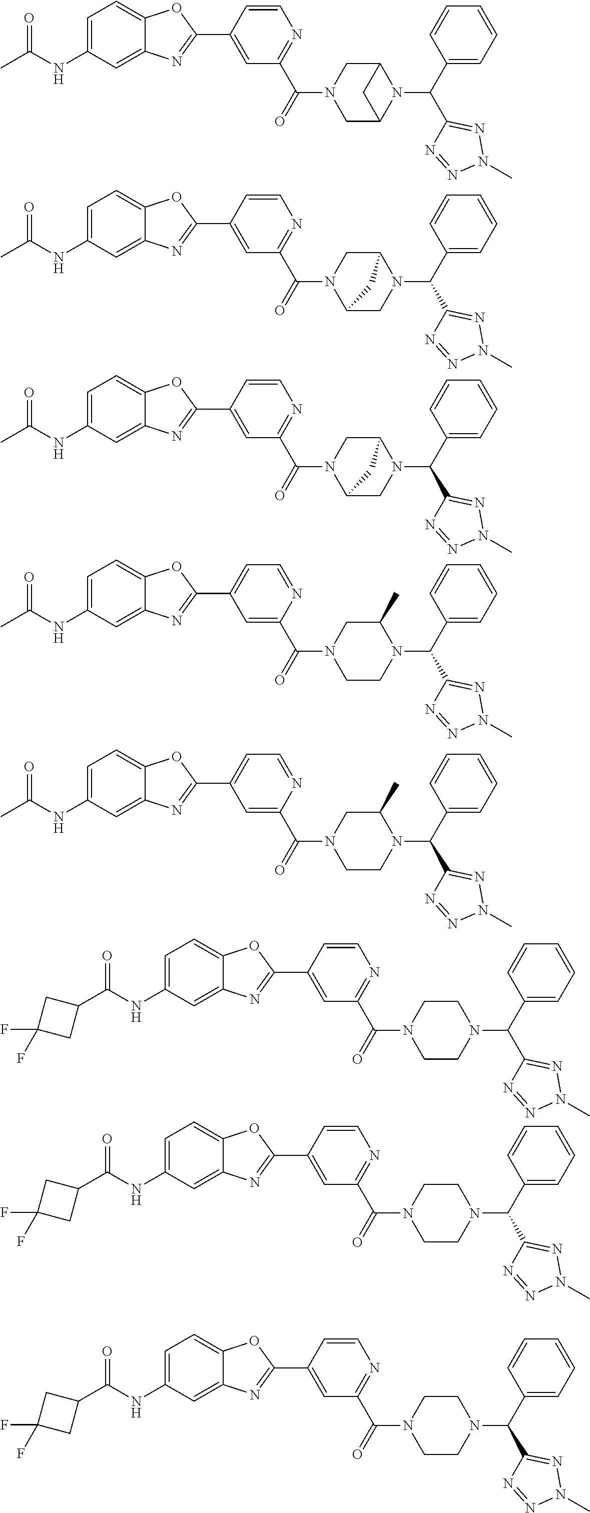

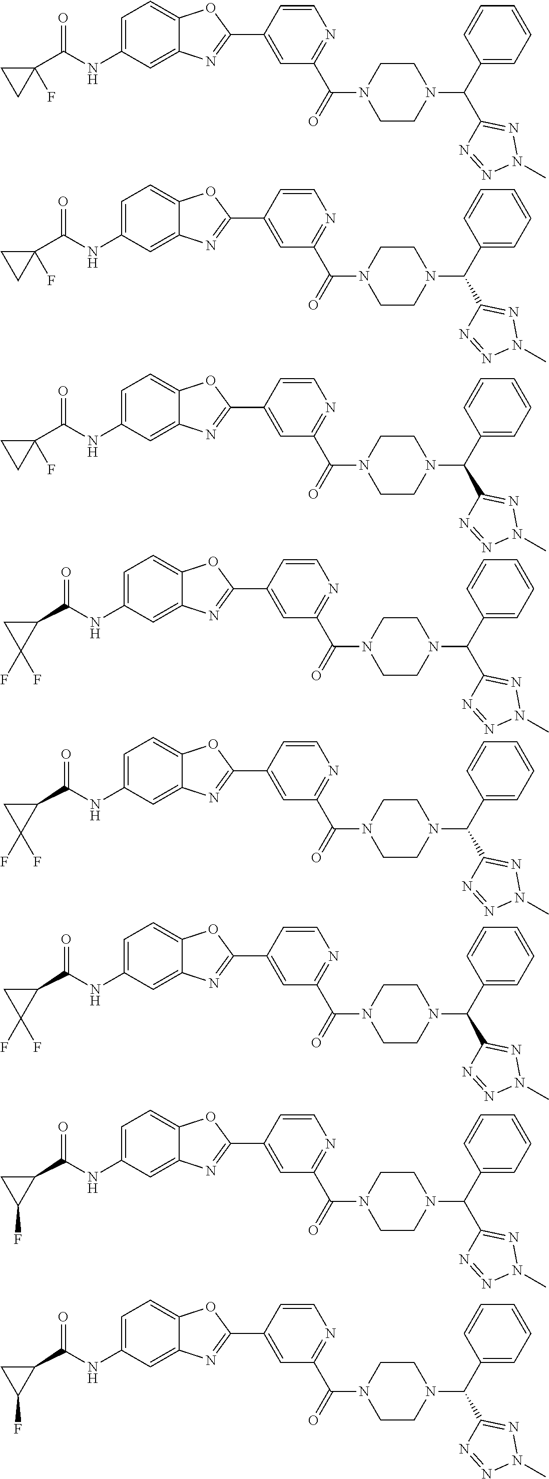

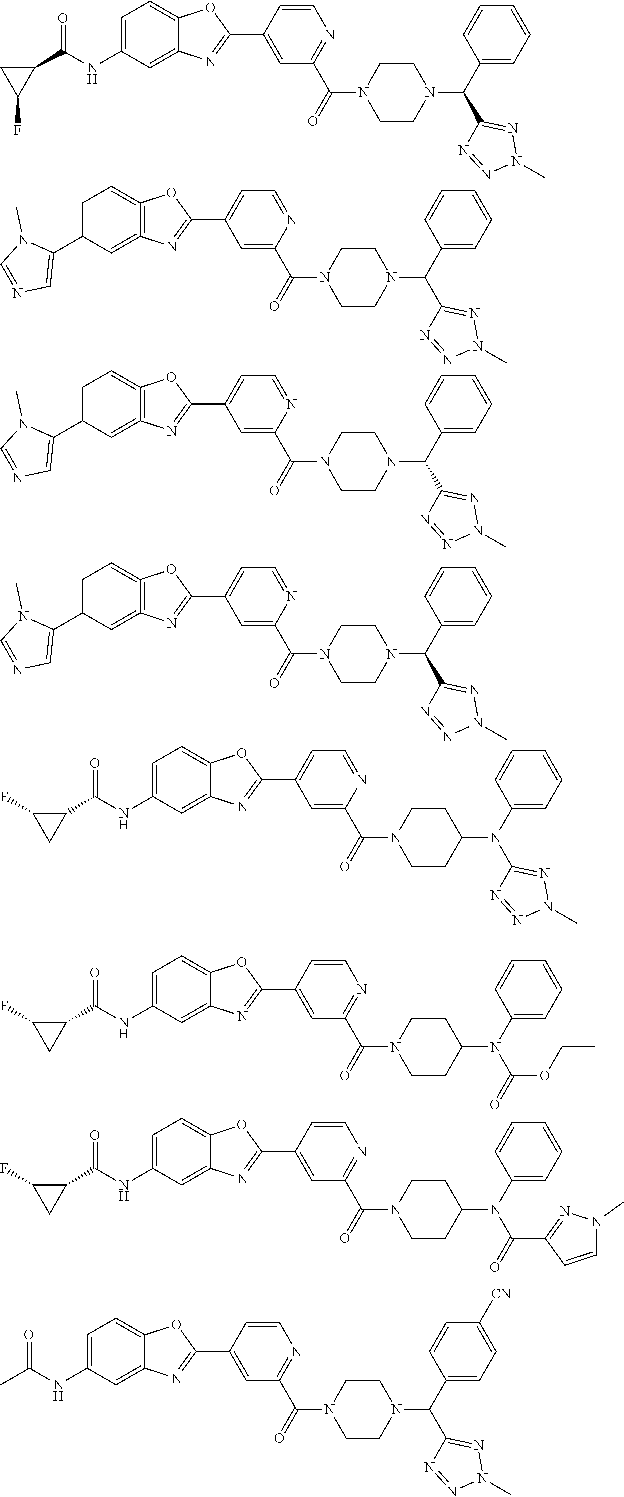

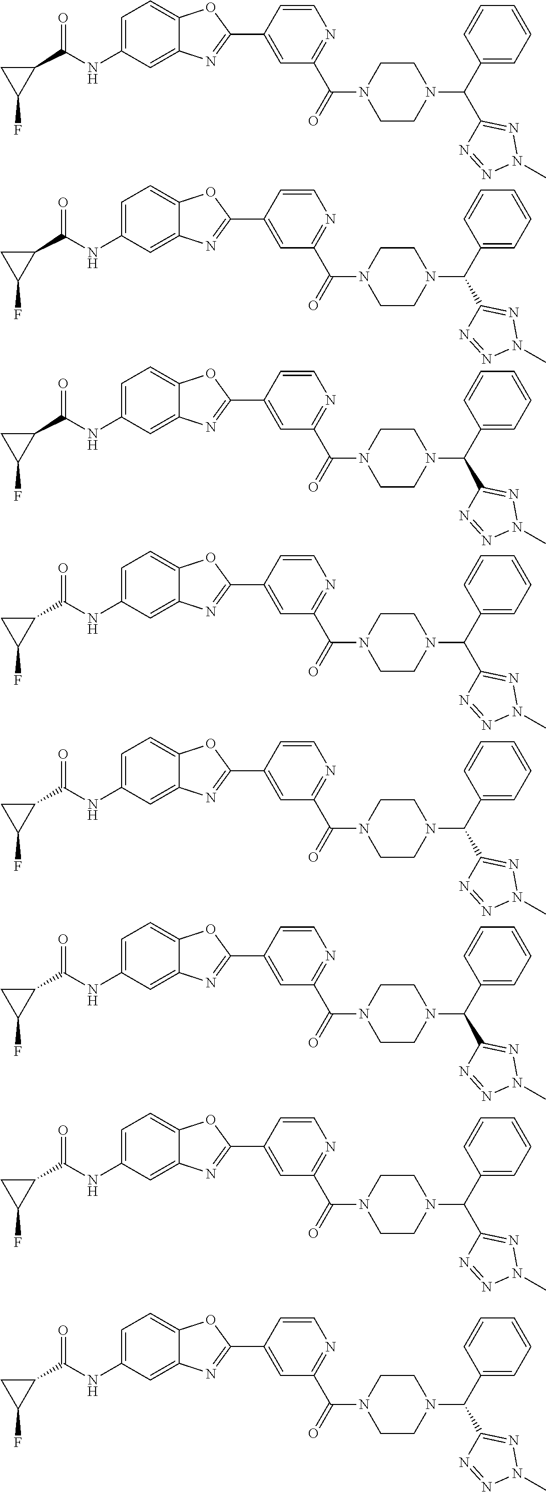

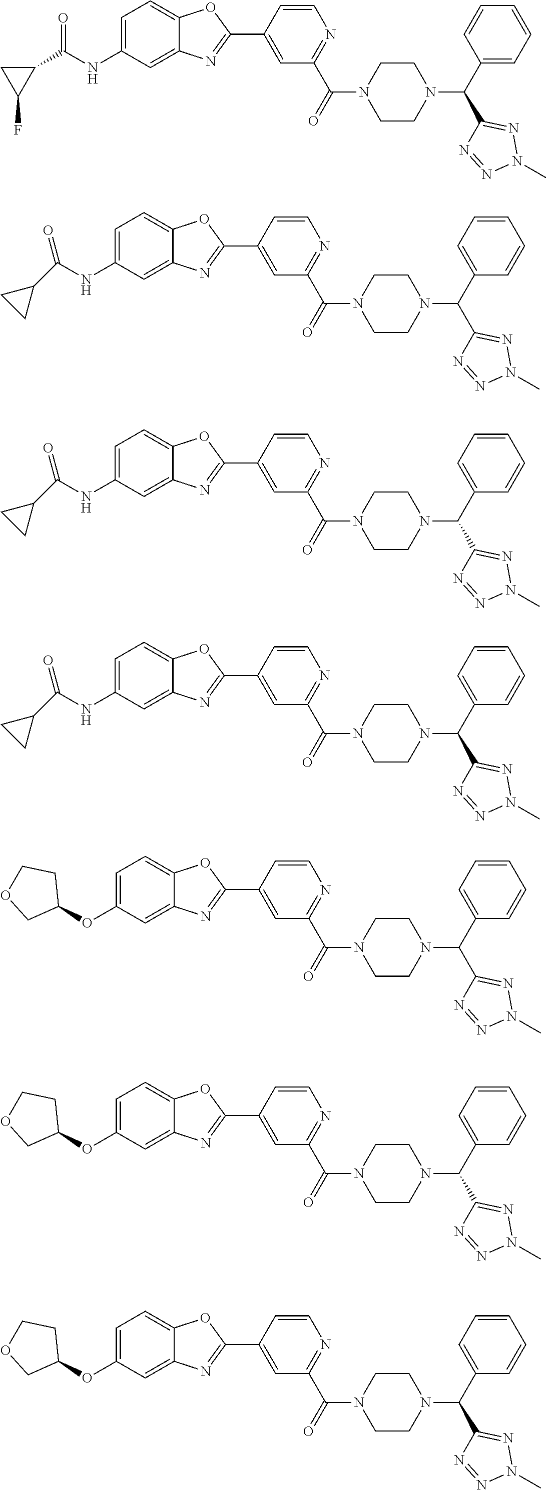

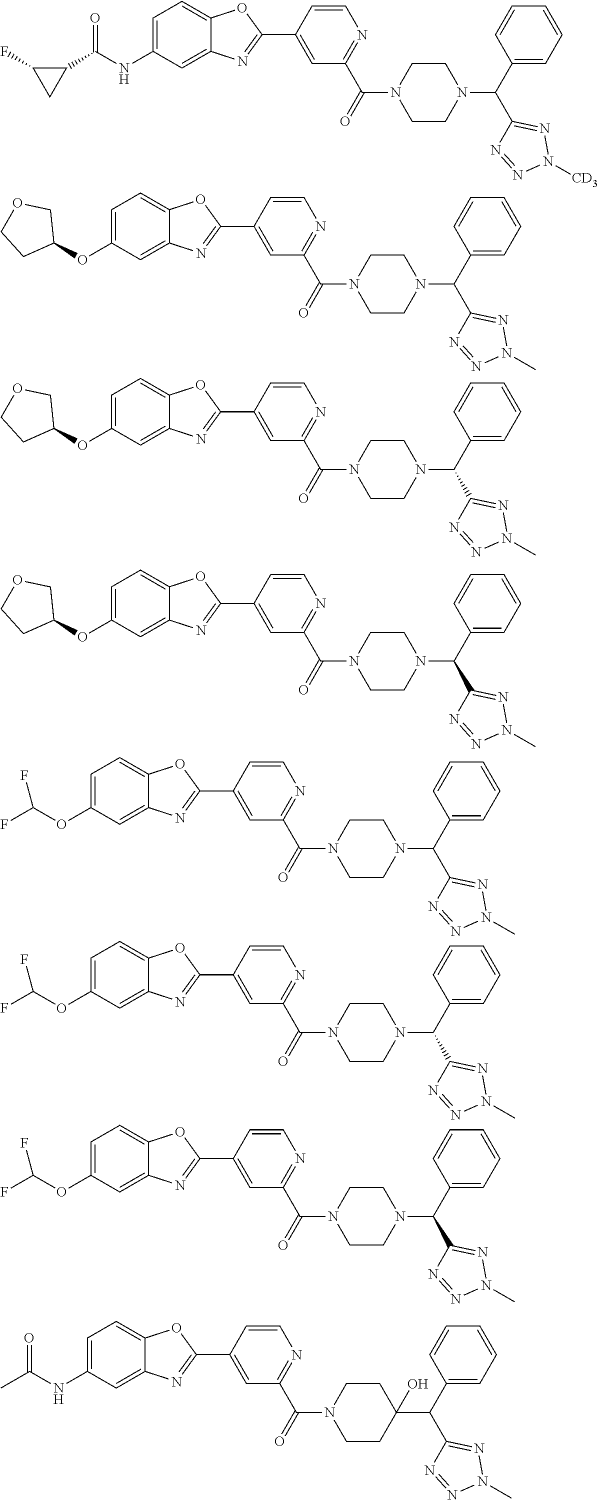

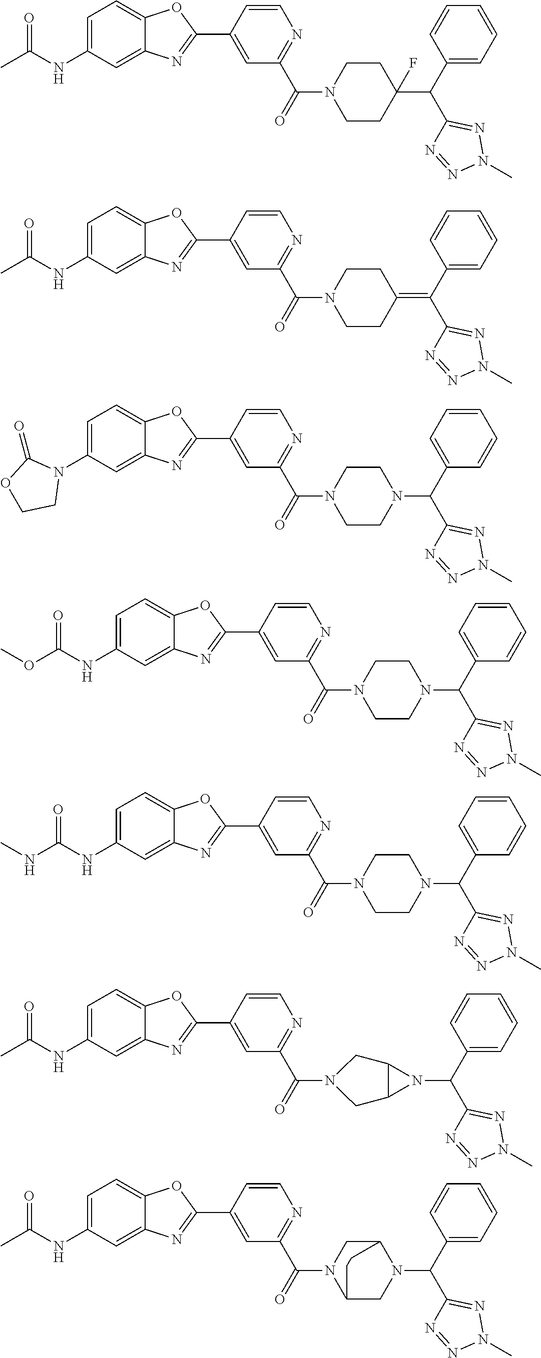

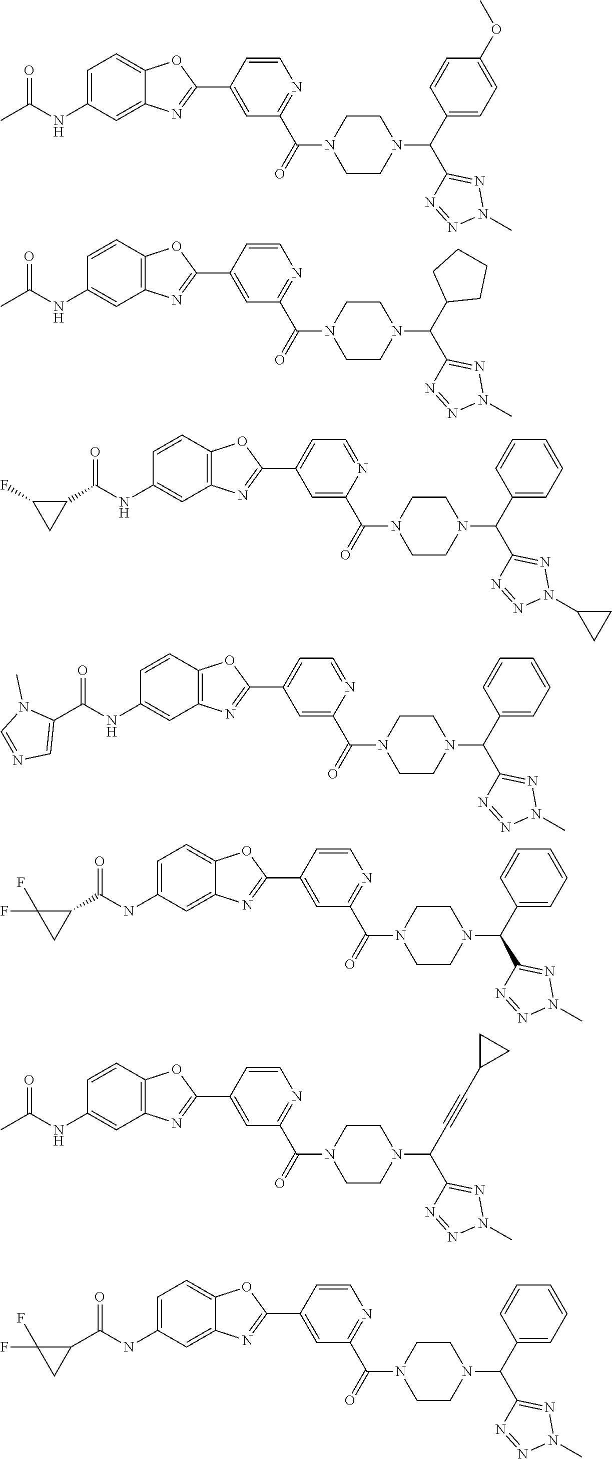

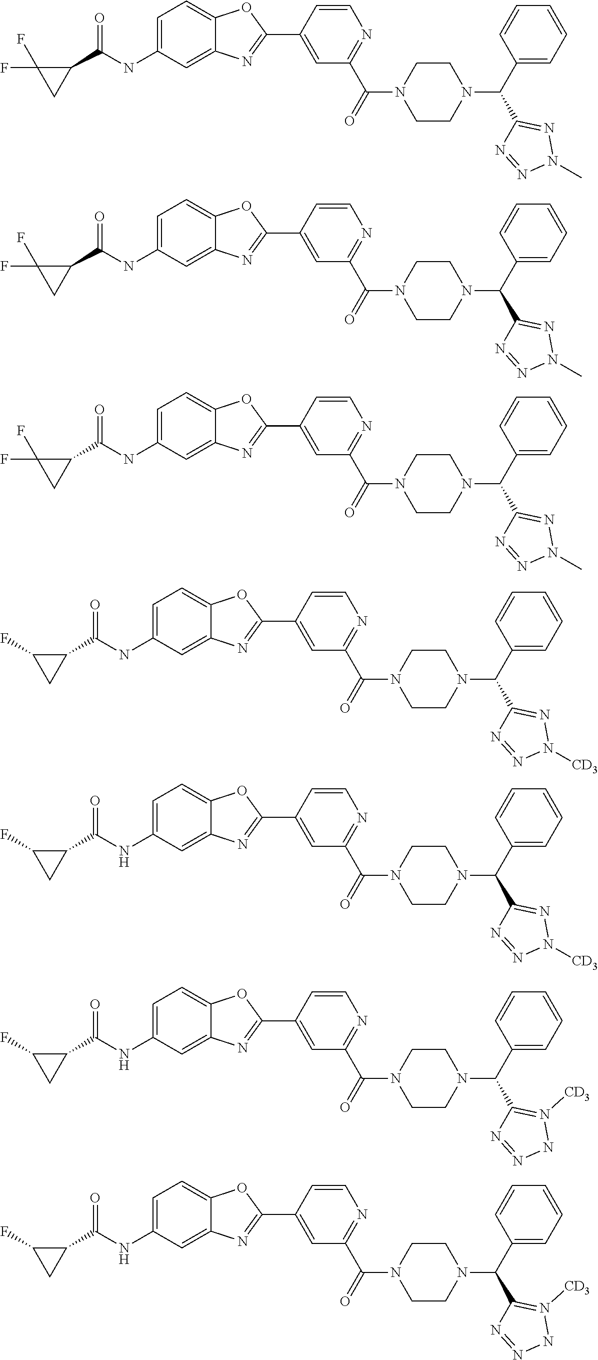

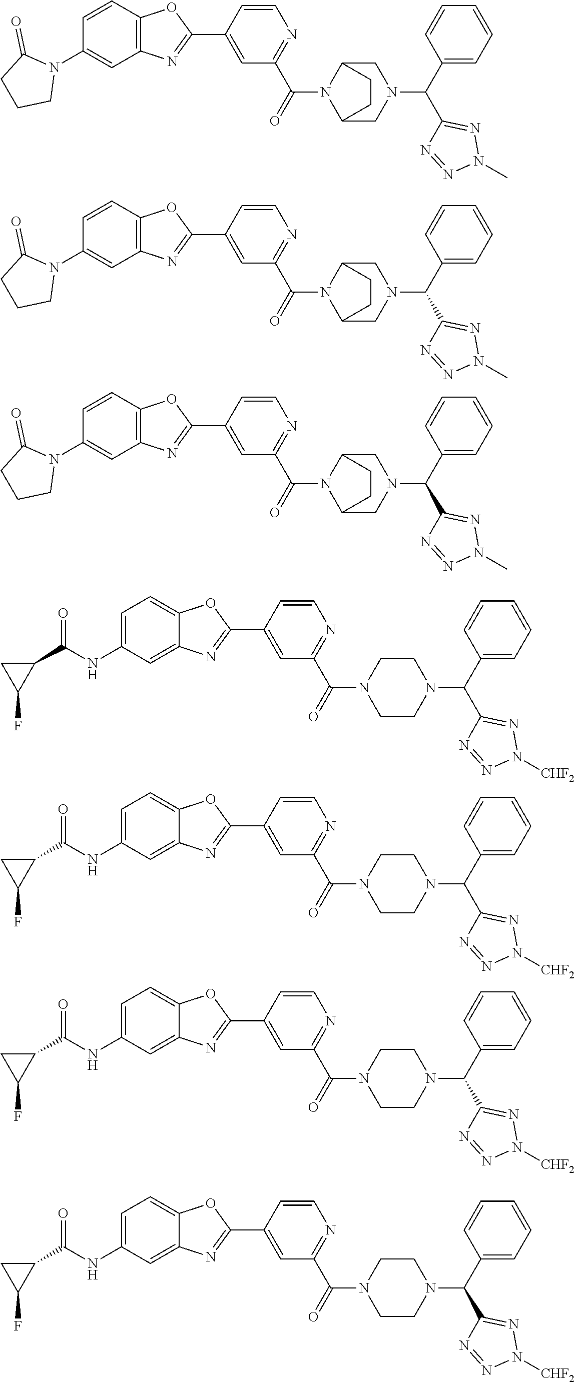

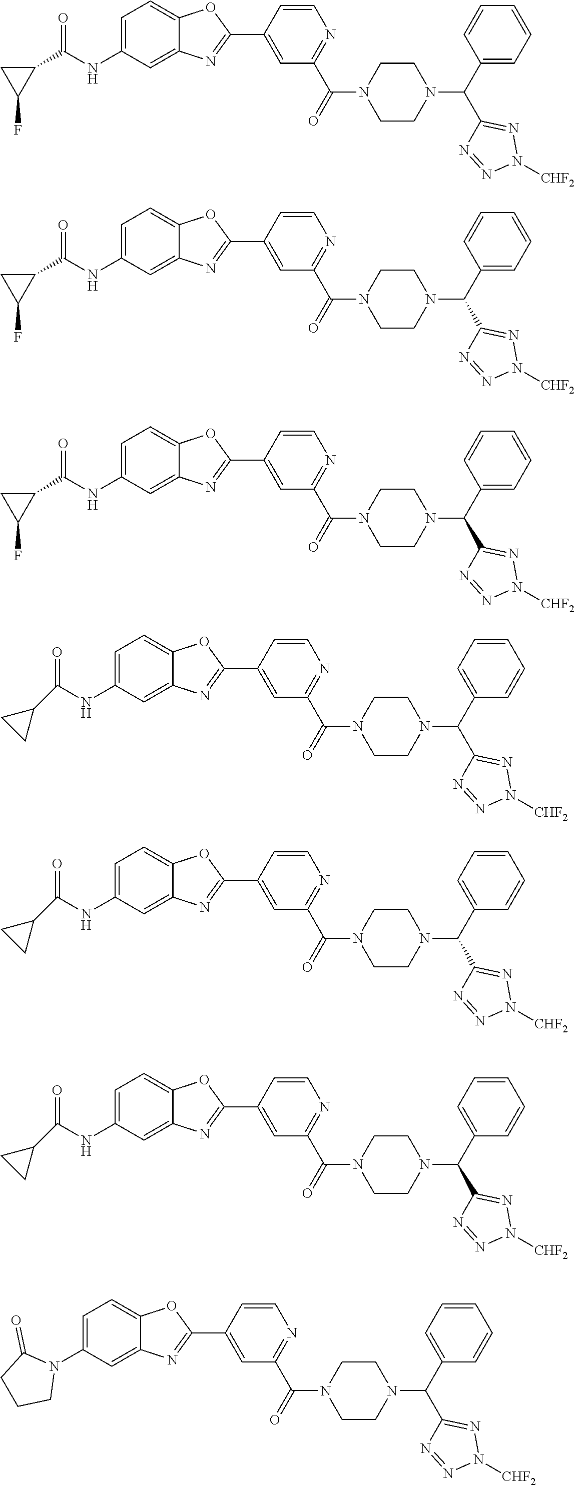

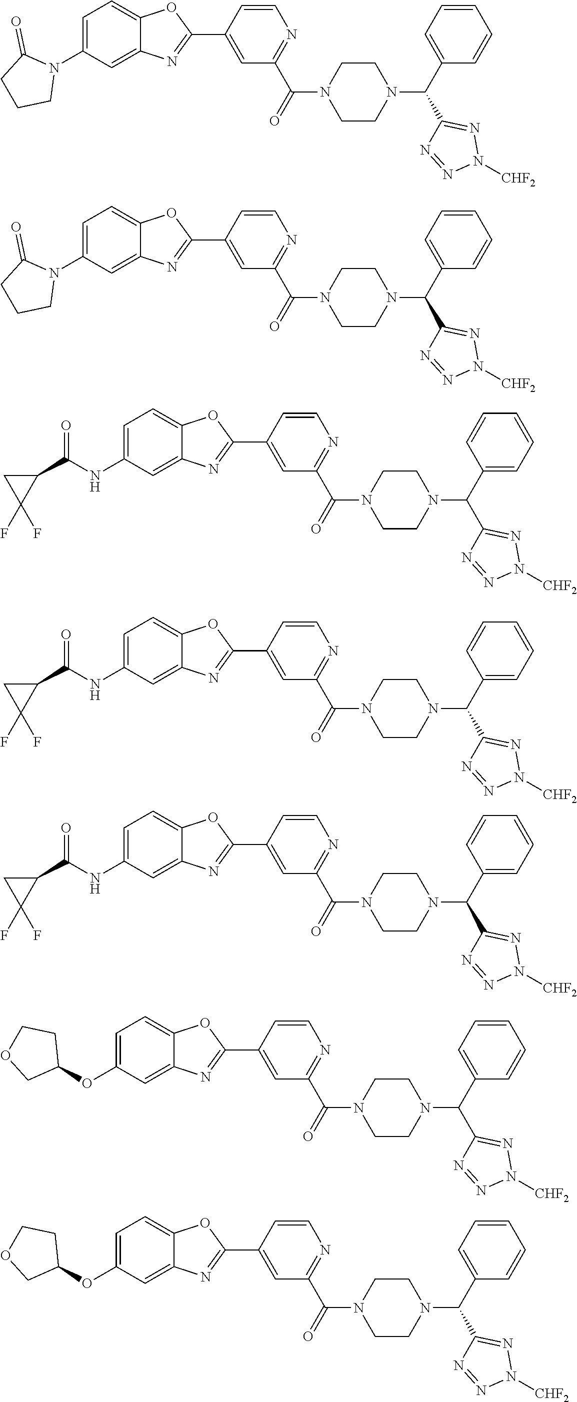

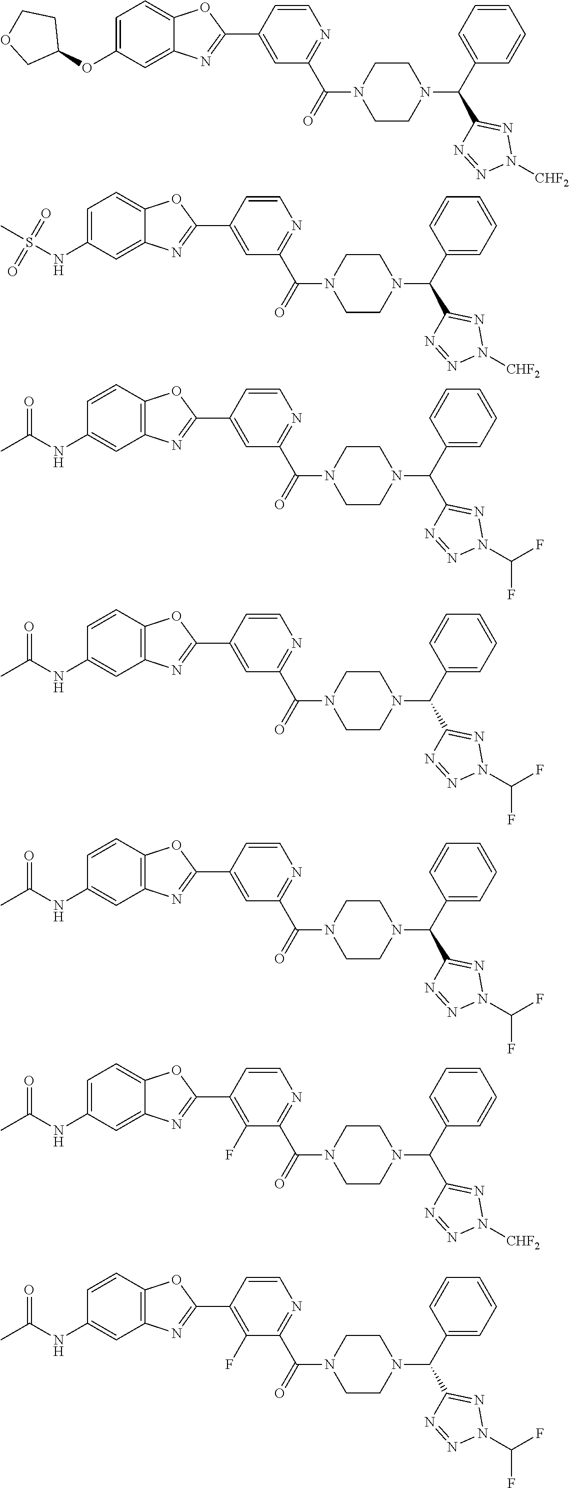

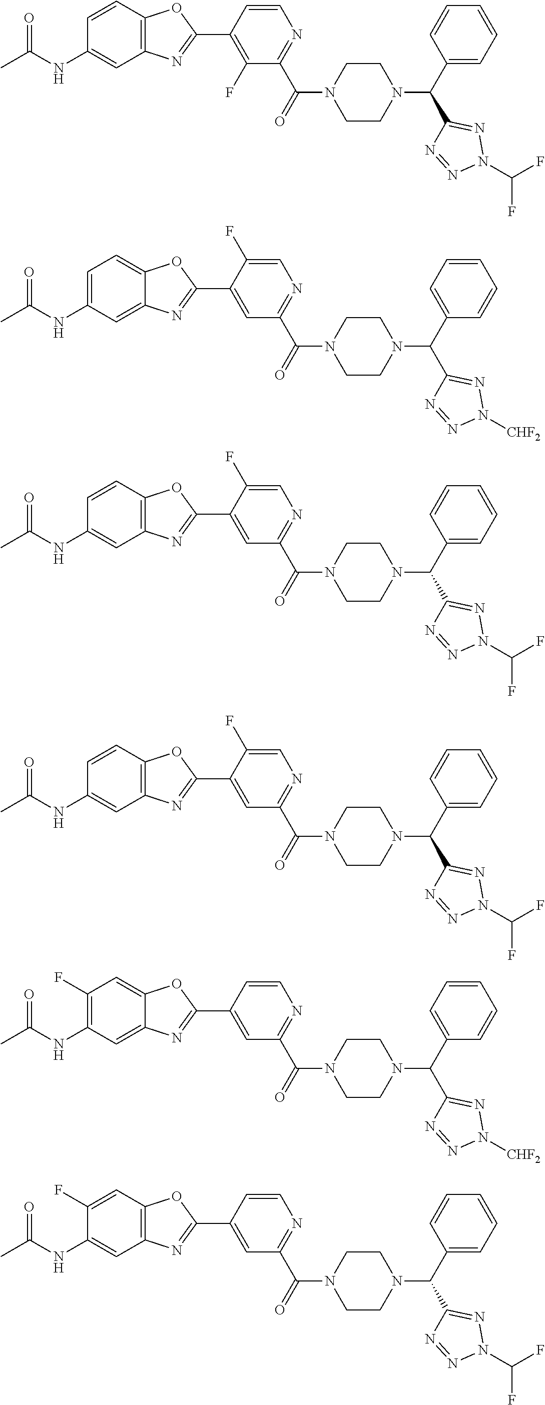

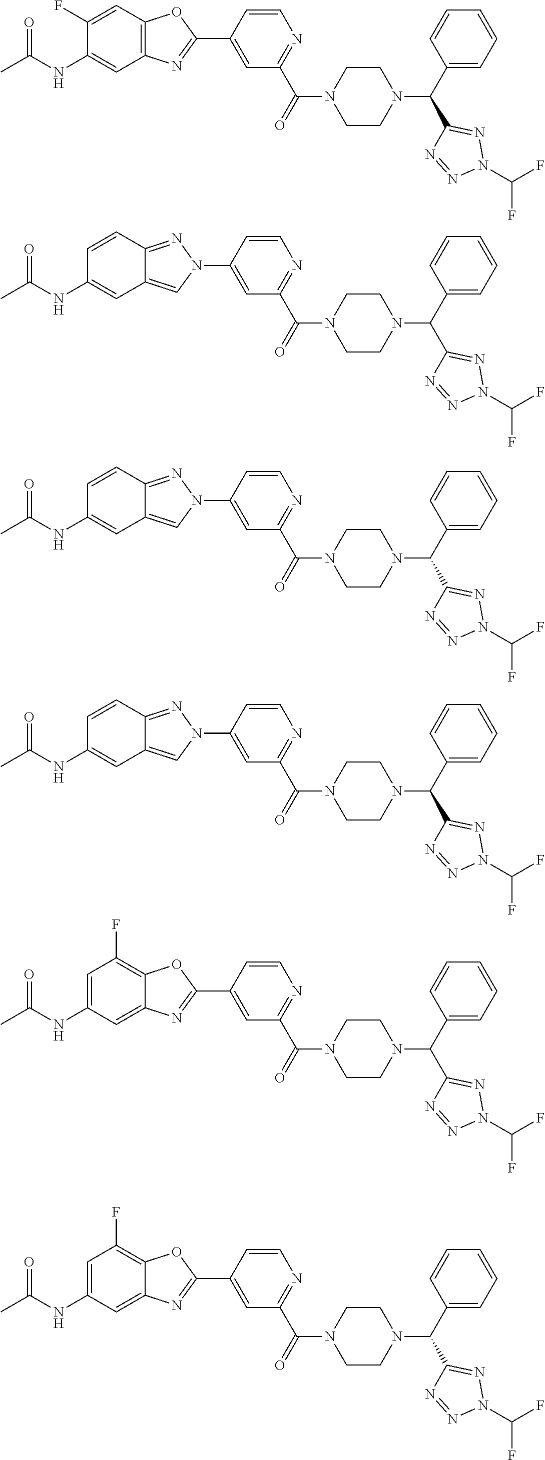

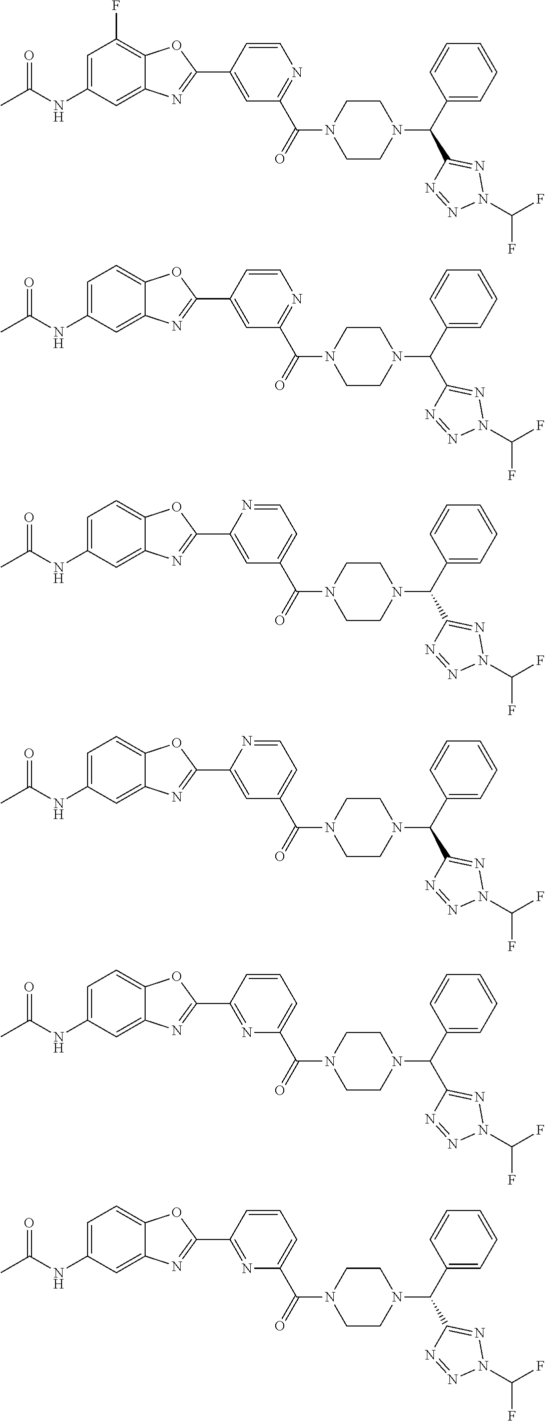

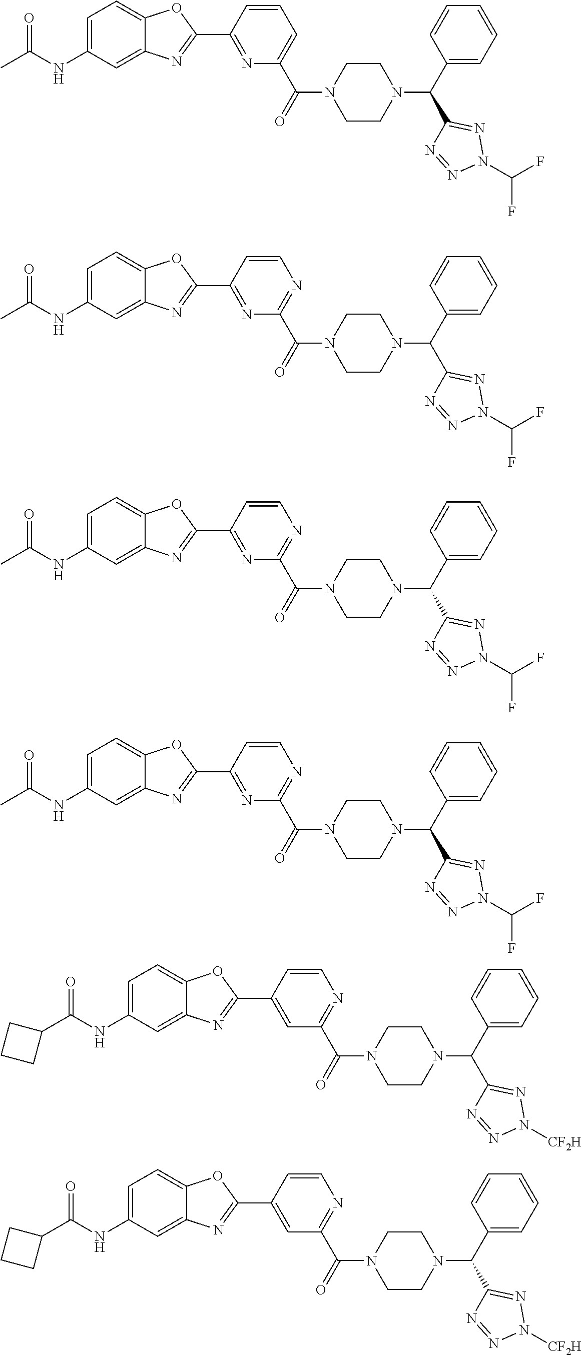

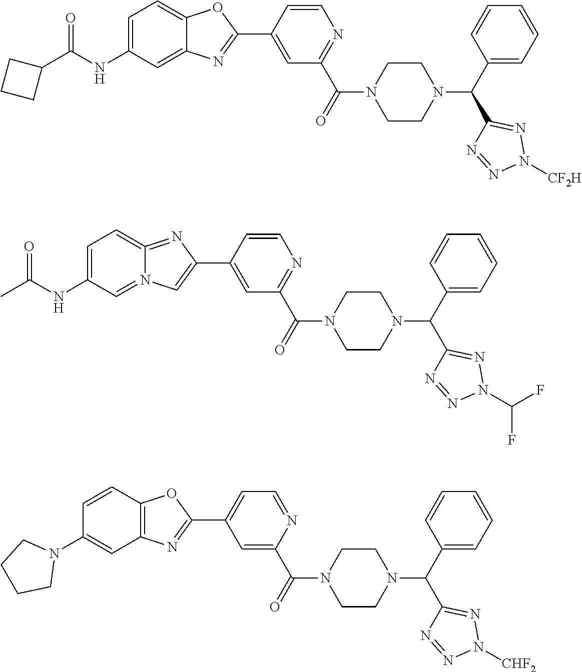

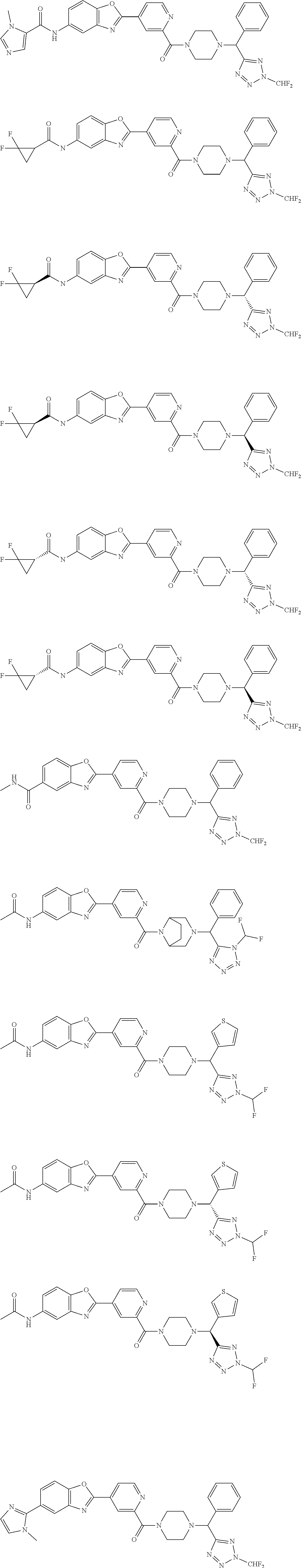

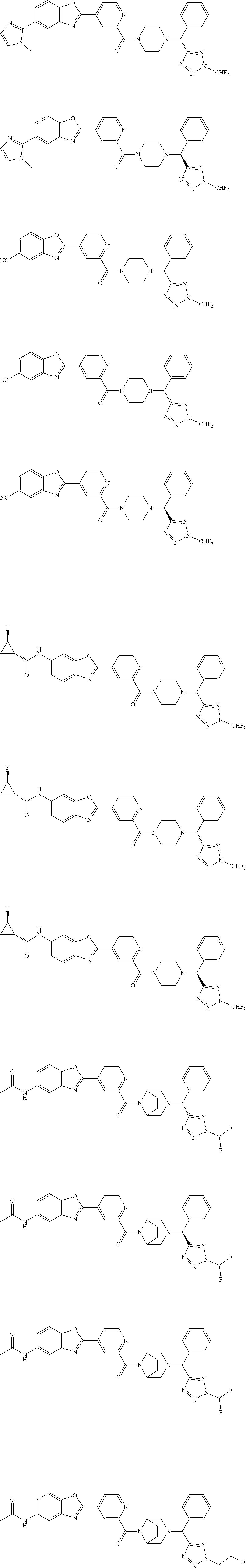

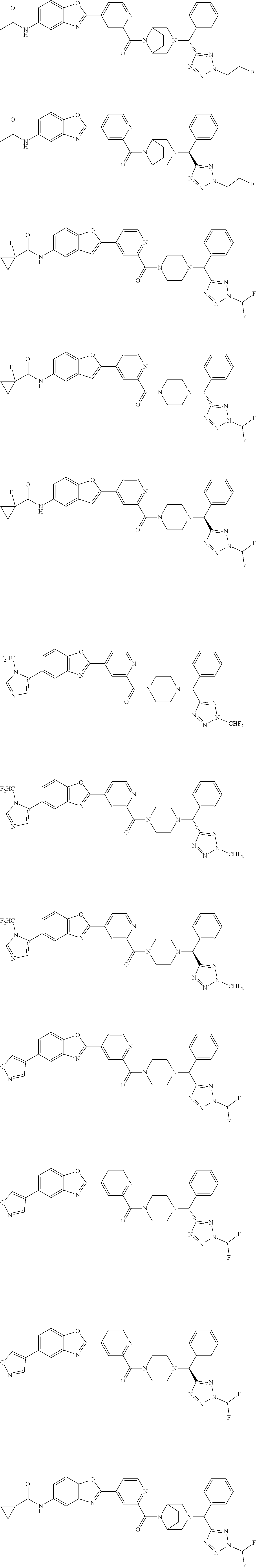

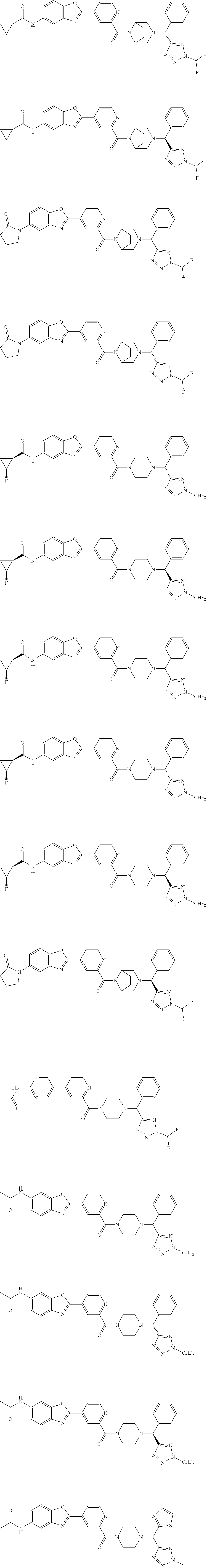

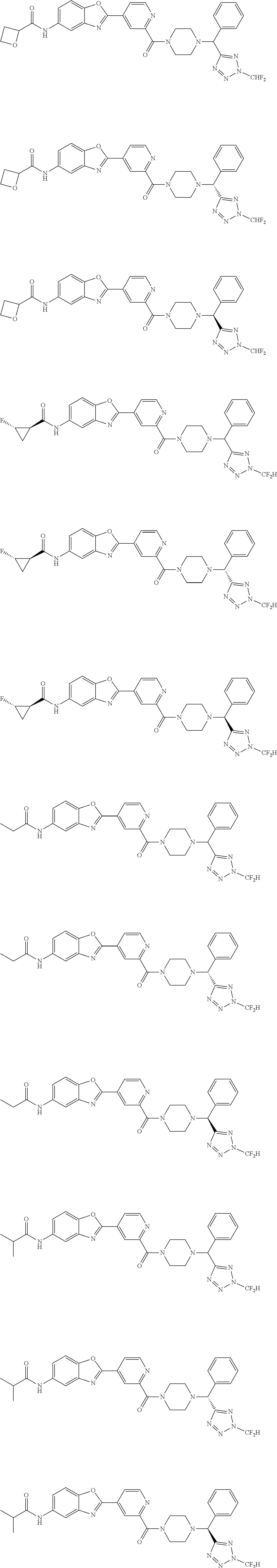

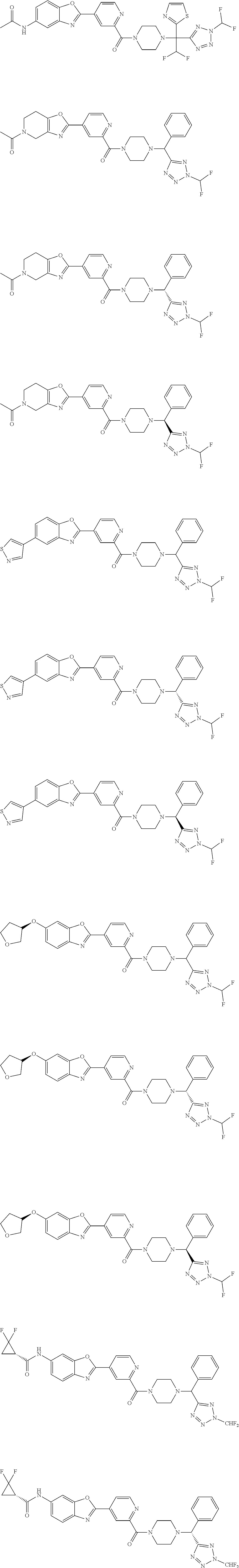

[chemical structure]

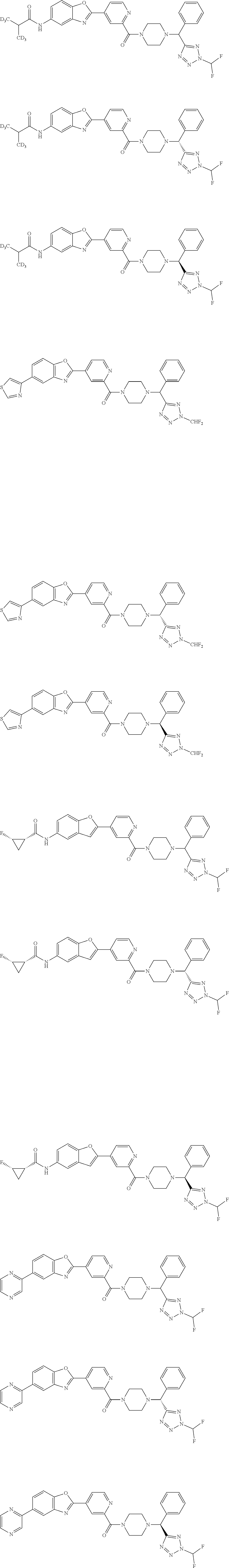

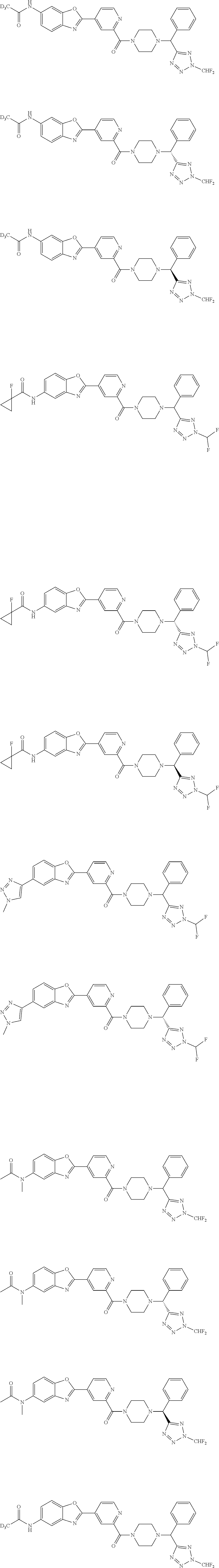

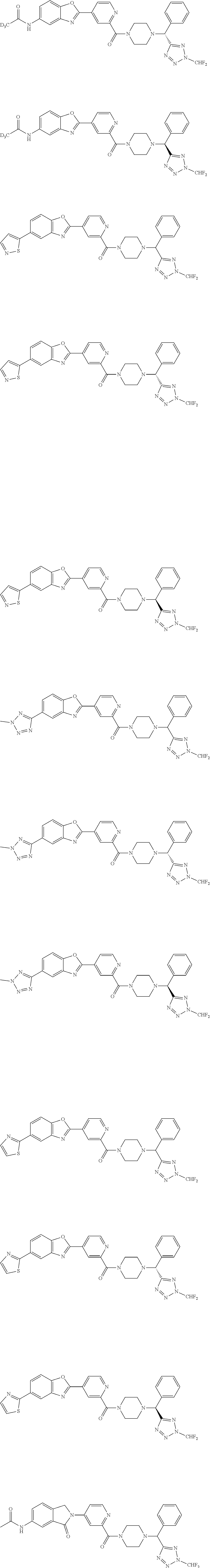

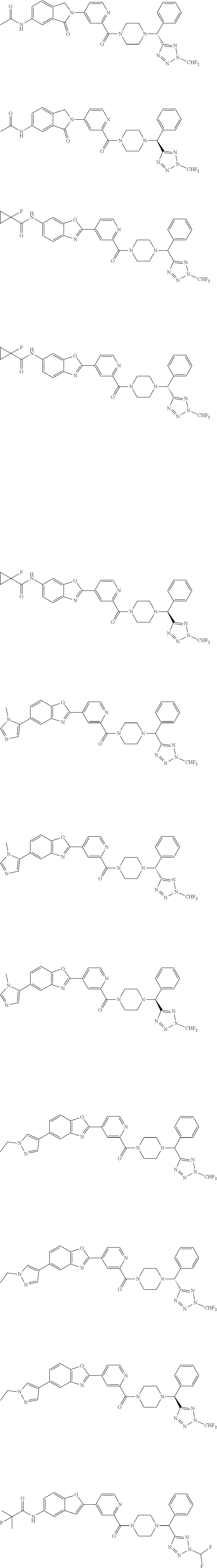

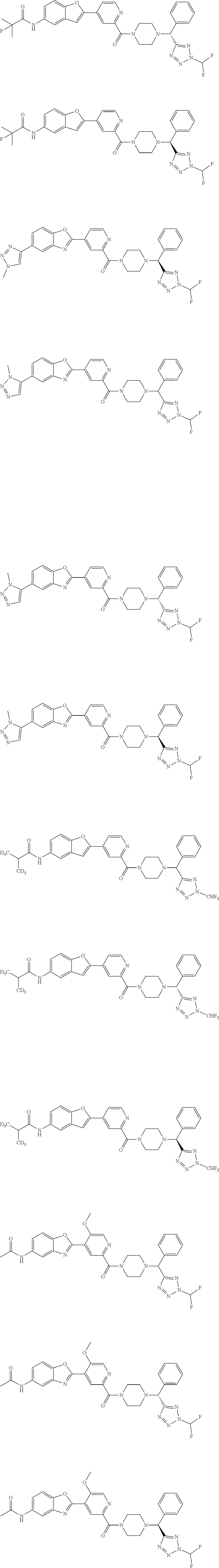

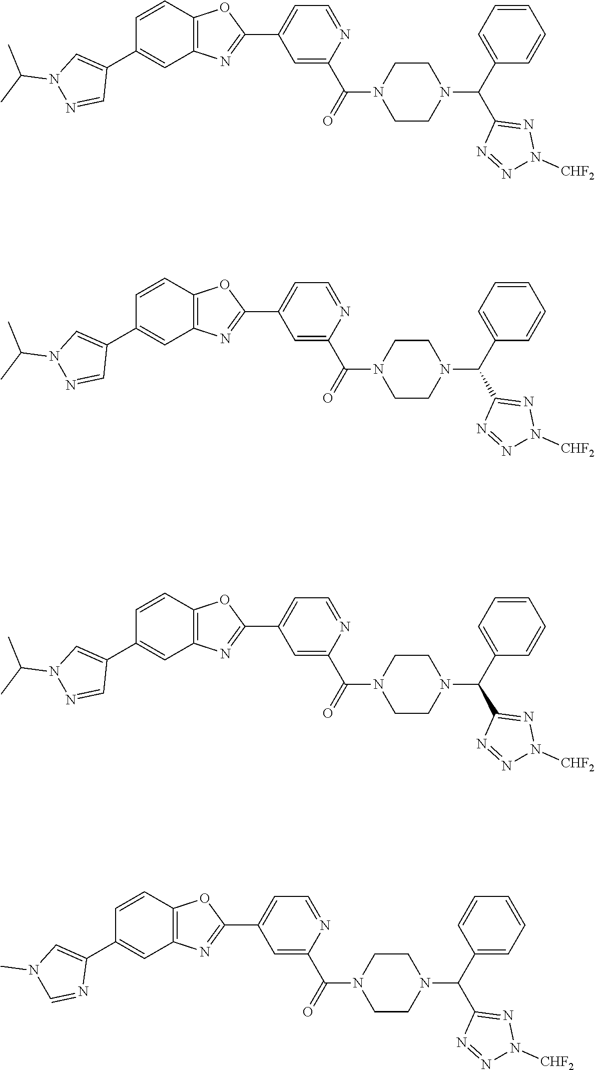 [SMILES: CC(C)n1cc(-c2ccc3oc(-c4ccnc(C(=O)N5CCN(C(c6ccccc6)c6nnn(C(F)F)n6)CC5)c4)nc3c2)cn1 ; CC(C)n1cc(-c2ccc3oc(-c4ccnc(C(=O)N5CCN([C@H](c6ccccc6)c6nnn(C(F)F)n6)CC5)c4)nc3c2)cn1 ; CC(C)n1cc(-c2ccc3oc(-c4ccnc(C(=O)N5CCN([C@@H](c6ccccc6)c6nnn(C(F)F)n6)CC5)c4)nc3c2)cn1 ; Cn1cnc(-c2ccc3oc(-c4ccnc(C(=O)N5CCN(C(c6ccccc6)c6nnn(C(F)F)n6)CC5)c4)nc3c2)c1]

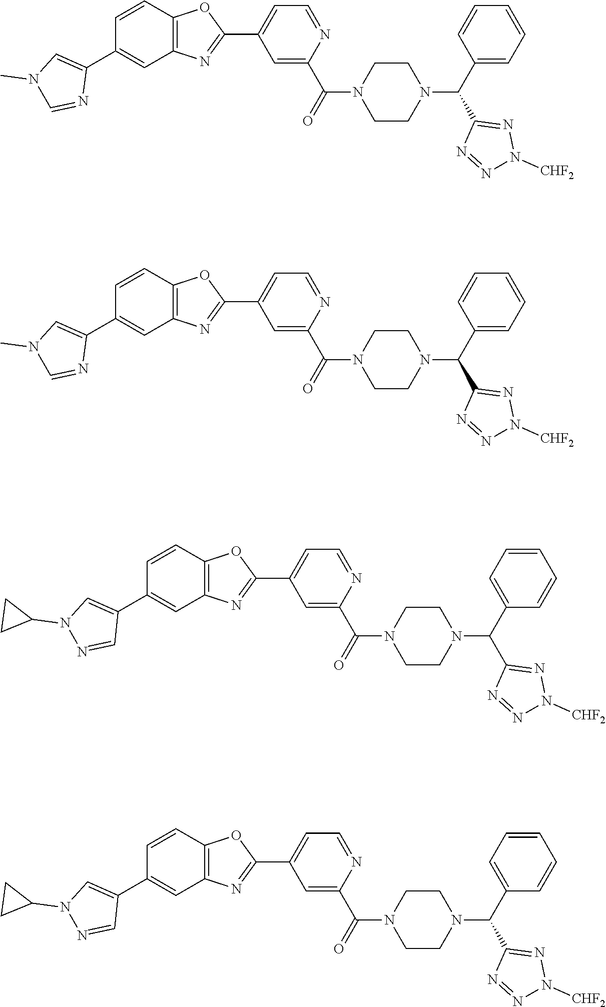 [SMILES: Cn1cnc(-c2ccc3oc(-c4ccnc(C(=O)N5CCN([C@H](c6ccccc6)c6nnn(C(F)F)n6)CC5)c4)nc3c2)c1 ; Cn1cnc(-c2ccc3oc(-c4ccnc(C(=O)N5CCN([C@@H](c6ccccc6)c6nnn(C(F)F)n6)CC5)c4)nc3c2)c1 ; O=C(c1cc(-c2nc3cc(-c4cnn(C5CC5)c4)ccc3o2)ccn1)N1CCN(C(c2ccccc2)c2nnn(C(F)F)n2)CC1 ; O=C(c1cc(-c2nc3cc(-c4cnn(C5CC5)c4)ccc3o2)ccn1)N1CCN([C@H](c2ccccc2)c2nnn(C(F)F)n2)CC1]

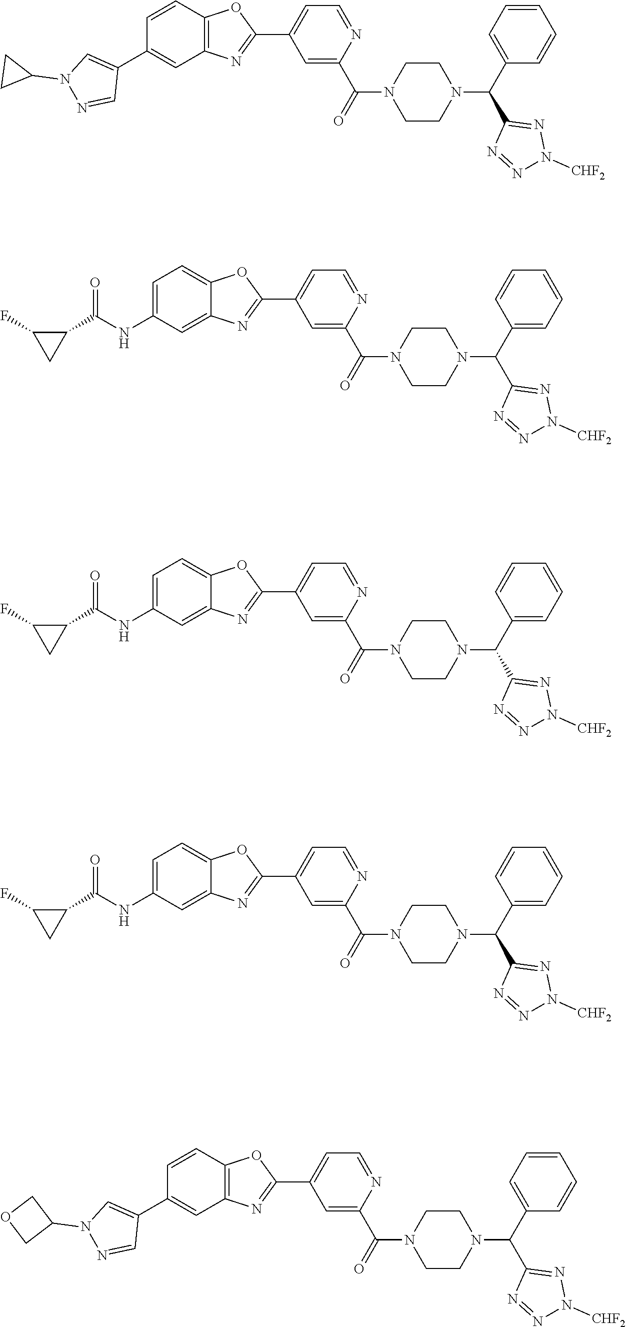 [SMILES: O=C(c1cc(-c2nc3cc(-c4cnn(C5CC5)c4)ccc3o2)ccn1)N1CCN([C@@H](c2ccccc2)c2nnn(C(F)F)n2)CC1 ; O=C(Nc1ccc2oc(-c3ccnc(C(=O)N4CCN(C(c5ccccc5)c5nnn(C(F)F)n5)CC4)c3)nc2c1)[C@@H]1C[C@@H]1F ; O=C(Nc1ccc2oc(-c3ccnc(C(=O)N4CCN([C@H](c5ccccc5)c5nnn(C(F)F)n5)CC4)c3)nc2c1)[C@@H]1C[C@@H]1F ; O=C(Nc1ccc2oc(-c3ccnc(C(=O)N4CCN([C@@H](c5ccccc5)c5nnn(C(F)F)n5)CC4)c3)nc2c1)[C@@H]1C[C@@H]1F ; O=C(c1cc(-c2nc3cc(-c4cnn(C5COC5)c4)ccc3o2)ccn1)N1CCN(C(c2ccccc2)c2nnn(C(F)F)n2)CC1]

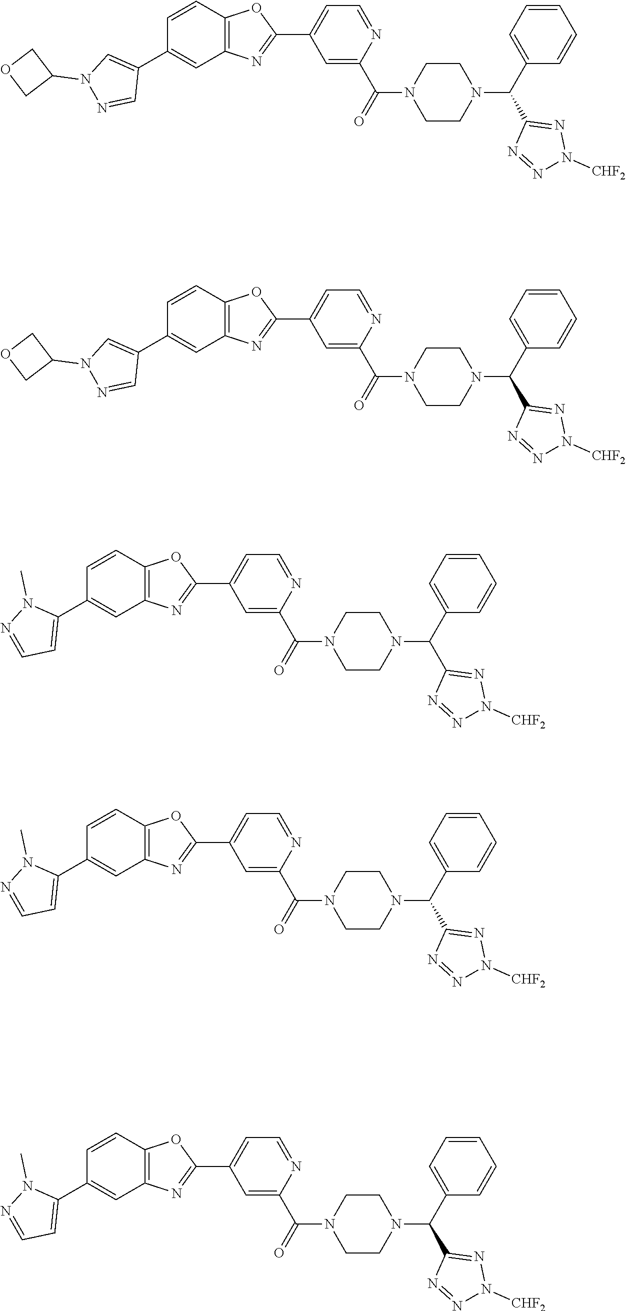 [SMILES: O=C(c1cc(-c2nc3cc(-c4cnn(C5COC5)c4)ccc3o2)ccn1)N1CCN([C@H](c2ccccc2)c2nnn(C(F)F)n2)CC1 ; O=C(c1cc(-c2nc3cc(-c4cnn(C5COC5)c4)ccc3o2)ccn1)N1CCN([C@@H](c2ccccc2)c2nnn(C(F)F)n2)CC1 ; Cn1nccc1-c1ccc2oc(-c3ccnc(C(=O)N4CCN(C(c5ccccc5)c5nnn(C(F)F)n5)CC4)c3)nc2c1 ; Cn1nccc1-c1ccc2oc(-c3ccnc(C(=O)N4CCN([C@H](c5ccccc5)c5nnn(C(F)F)n5)CC4)c3)nc2c1 ; Cn1nccc1-c1ccc2oc(-c3ccnc(C(=O)N4CCN([C@@H](c5ccccc5)c5nnn(C(F)F)n5)CC4)c3)nc2c1]

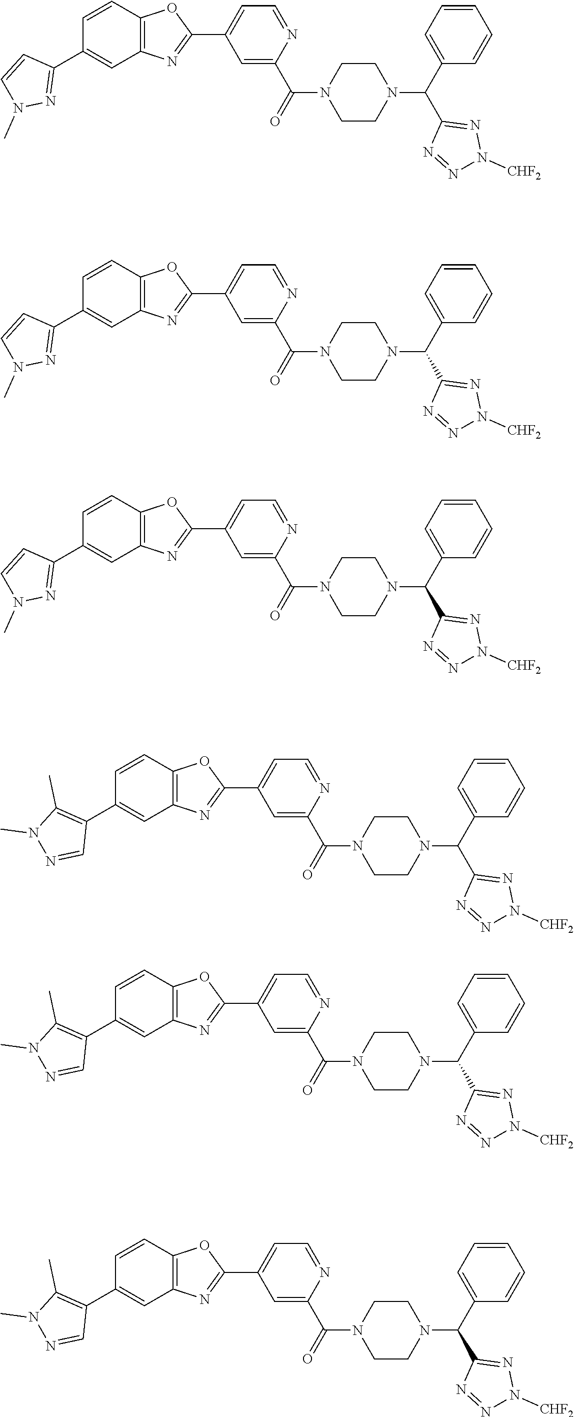 [SMILES: Cn1ccc(-c2ccc3oc(-c4ccnc(C(=O)N5CCN(C(c6ccccc6)c6nnn(C(F)F)n6)CC5)c4)nc3c2)n1 ; Cn1ccc(-c2ccc3oc(-c4ccnc(C(=O)N5CCN([C@H](c6ccccc6)c6nnn(C(F)F)n6)CC5)c4)nc3c2)n1 ; Cn1ccc(-c2ccc3oc(-c4ccnc(C(=O)N5CCN([C@@H](c6ccccc6)c6nnn(C(F)F)n6)CC5)c4)nc3c2)n1 ; Cc1c(-c2ccc3oc(-c4ccnc(C(=O)N5CCN(C(c6ccccc6)c6nnn(C(F)F)n6)CC5)c4)nc3c2)cnn1C ; Cc1c(-c2ccc3oc(-c4ccnc(C(=O)N5CCN([C@H](c6ccccc6)c6nnn(C(F)F)n6)CC5)c4)nc3c2)cnn1C ; Cc1c(-c2ccc3oc(-c4ccnc(C(=O)N5CCN([C@@H](c6ccccc6)c6nnn(C(F)F)n6)CC5)c4)nc3c2)cnn1C]

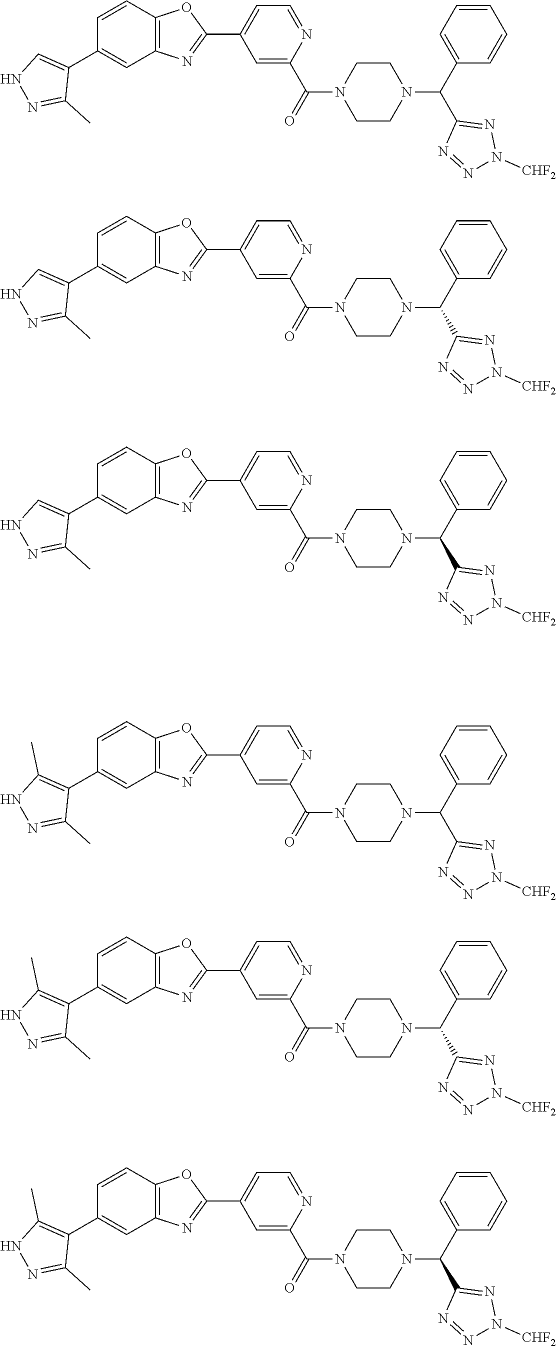

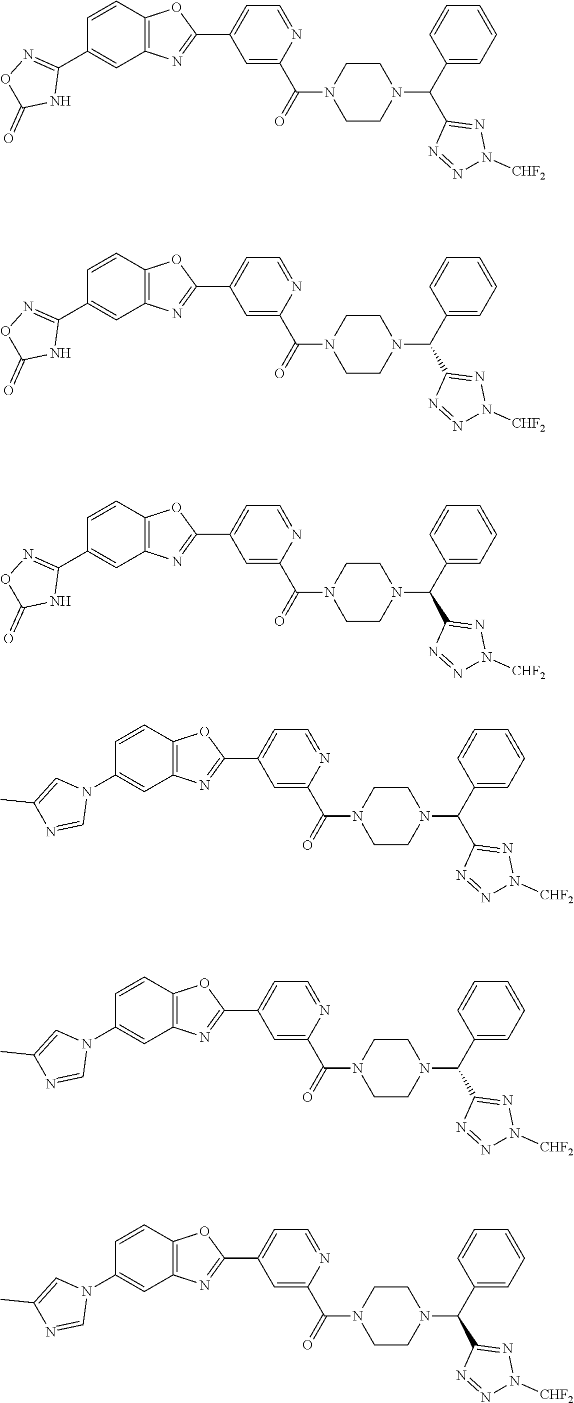

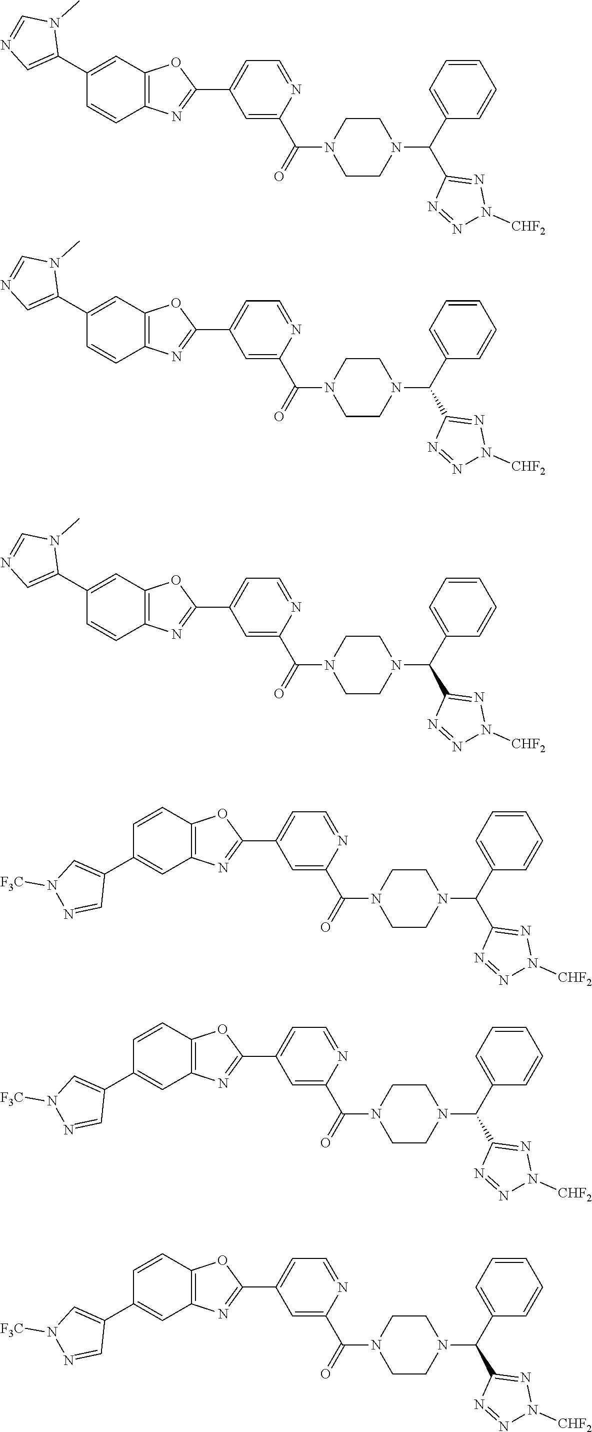

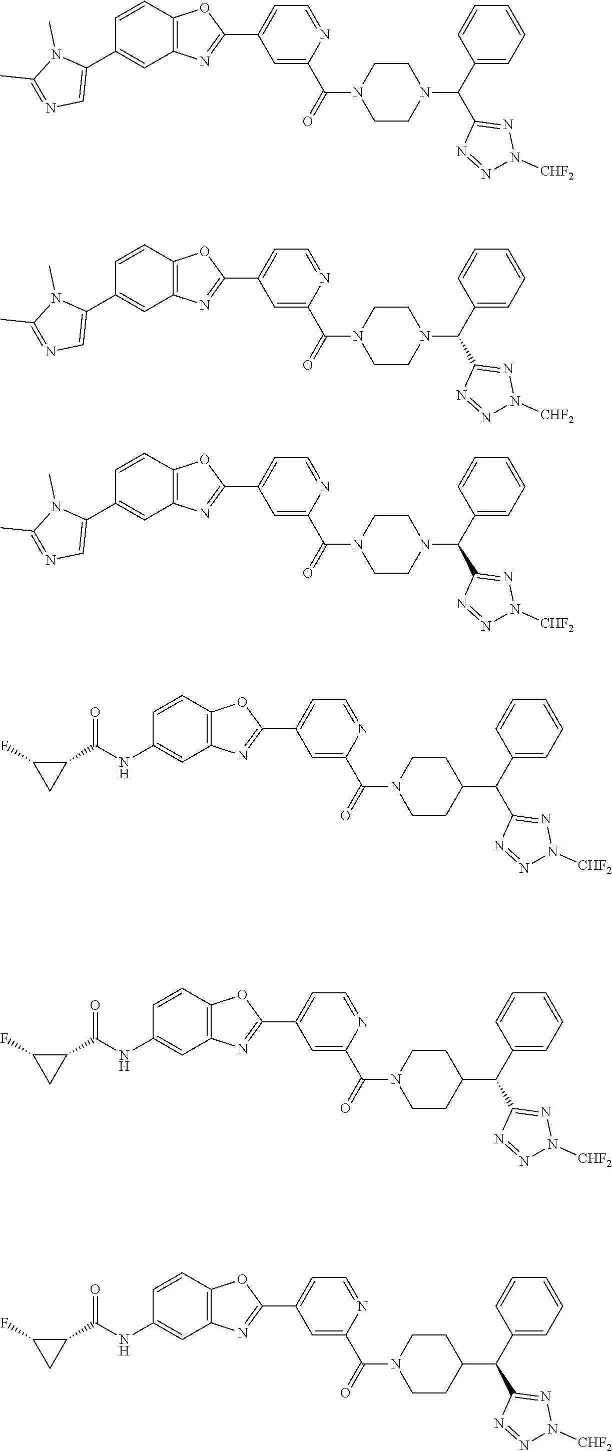

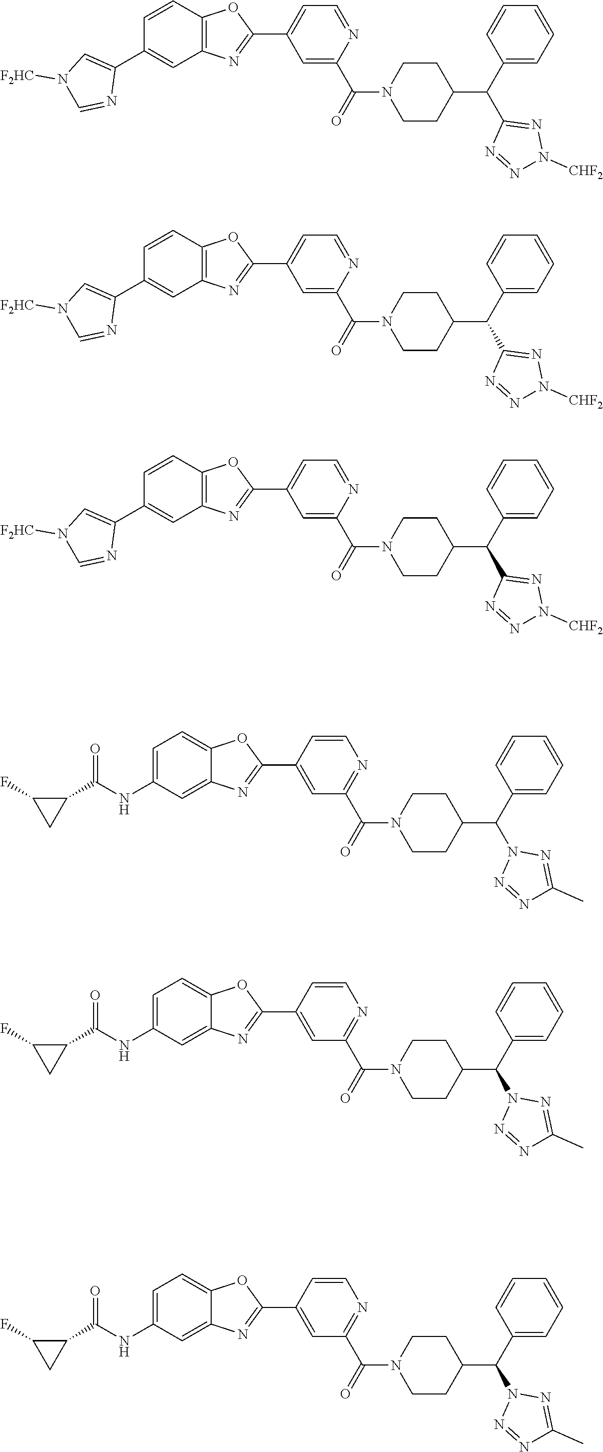

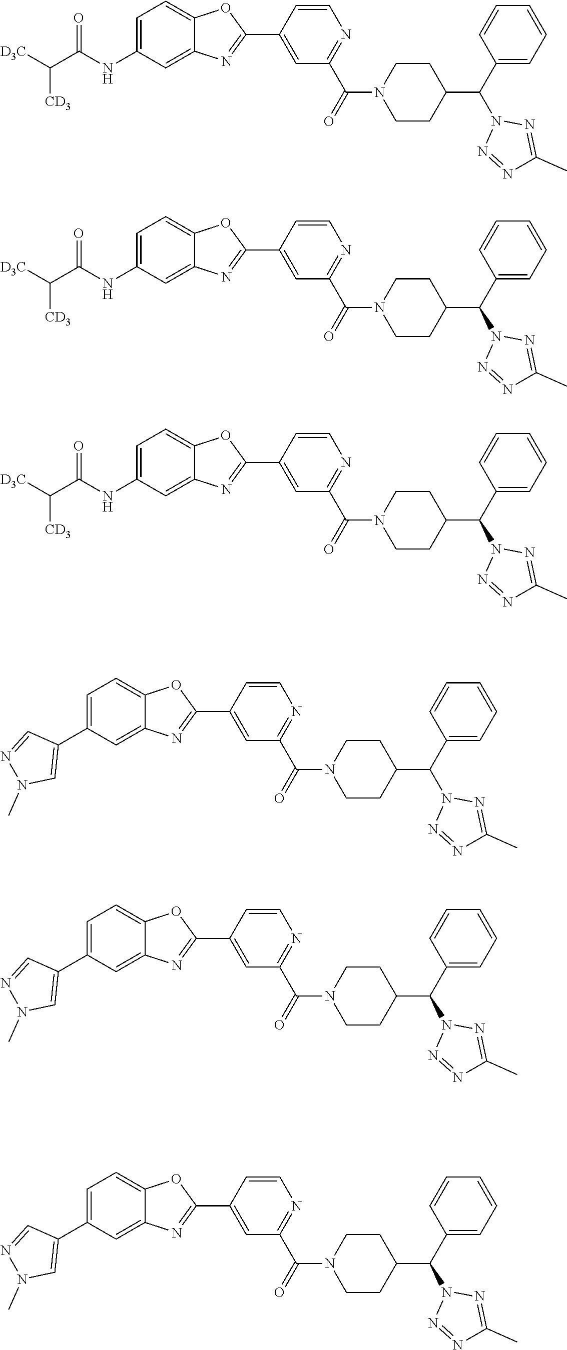

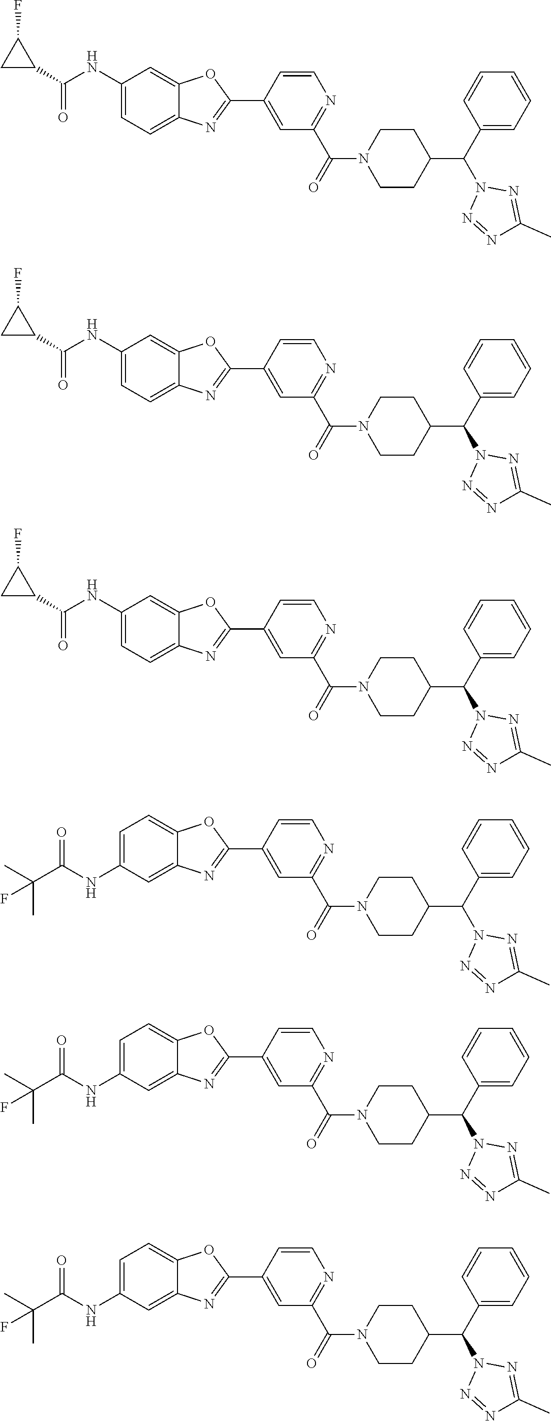

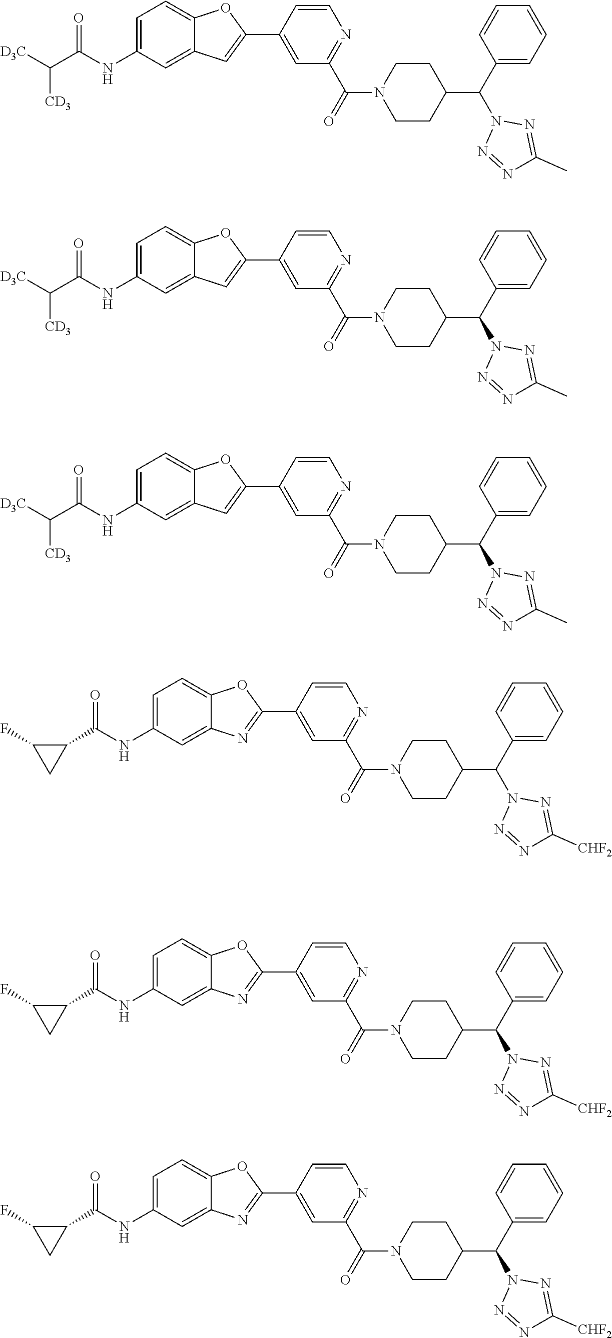

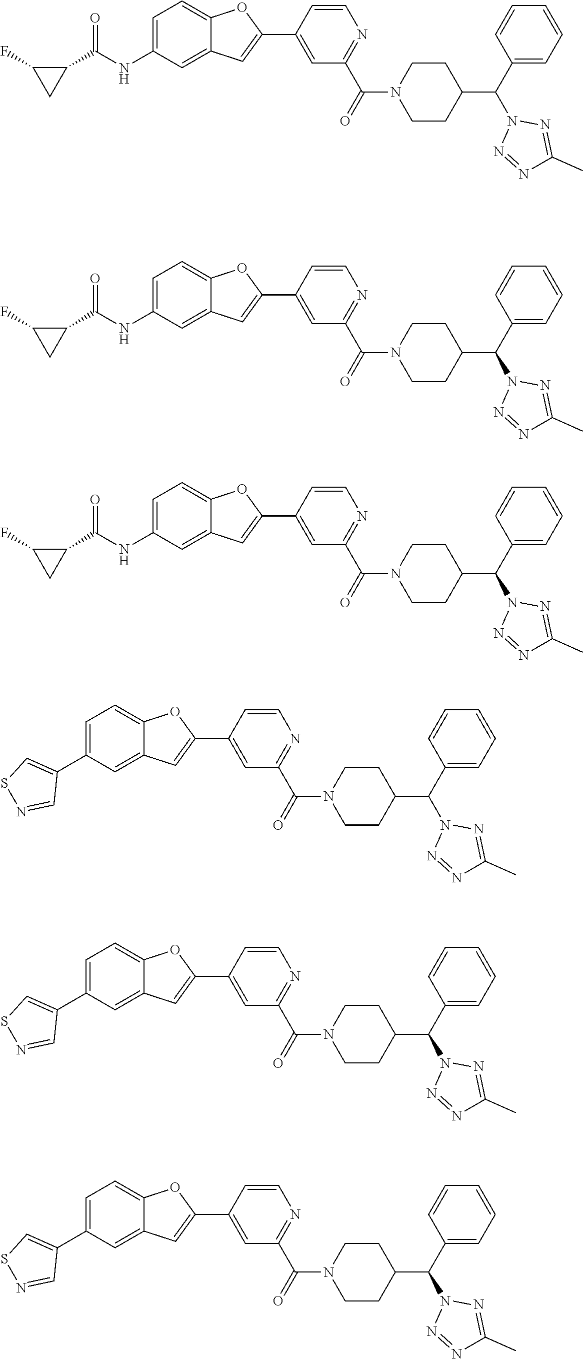 [SMILES: Cc1nnn(C(c2ccccc2)C2CCN(C(=O)c3cc(-c4cc5cc(NC(=O)[C@@H]6C[C@@H]6F)ccc5o4)ccn3)CC2)n1 ; Cc1nnn([C@H](c2ccccc2)C2CCN(C(=O)c3cc(-c4cc5cc(NC(=O)[C@@H]6C[C@@H]6F)ccc5o4)ccn3)CC2)n1 ; Cc1nnn([C@H](c2ccccc2)C2CCN(C(=O)c3cc(-c4cc5cc(NC(=O)[C@@H]6C[C@@H]6F)ccc5o4)ccn3)CC2)n1 ; Cc1nnn(C(c2ccccc2)C2CCN(C(=O)c3cc(-c4cc5cc(-c6cnsc6)ccc5o4)ccn3)CC2)n1 ; Cc1nnn([C@H](c2ccccc2)C2CCN(C(=O)c3cc(-c4cc5cc(-c6cnsc6)ccc5o4)ccn3)CC2)n1 ; Cc1nnn([C@H](c2ccccc2)C2CCN(C(=O)c3cc(-c4cc5cc(-c6cnsc6)ccc5o4)ccn3)CC2)n1]

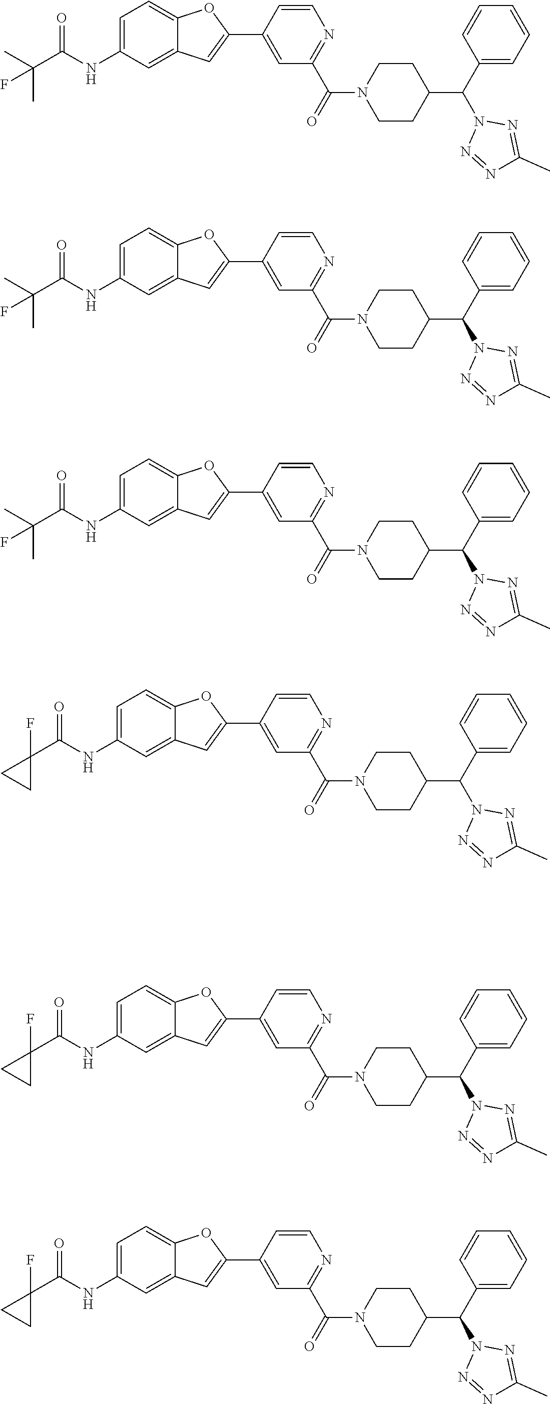

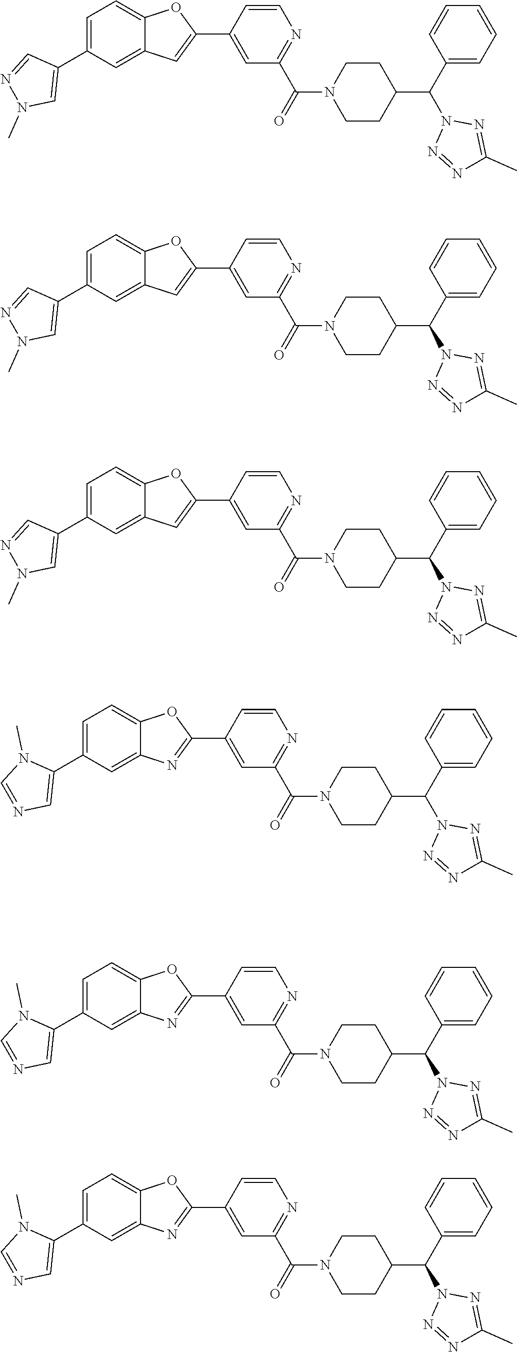

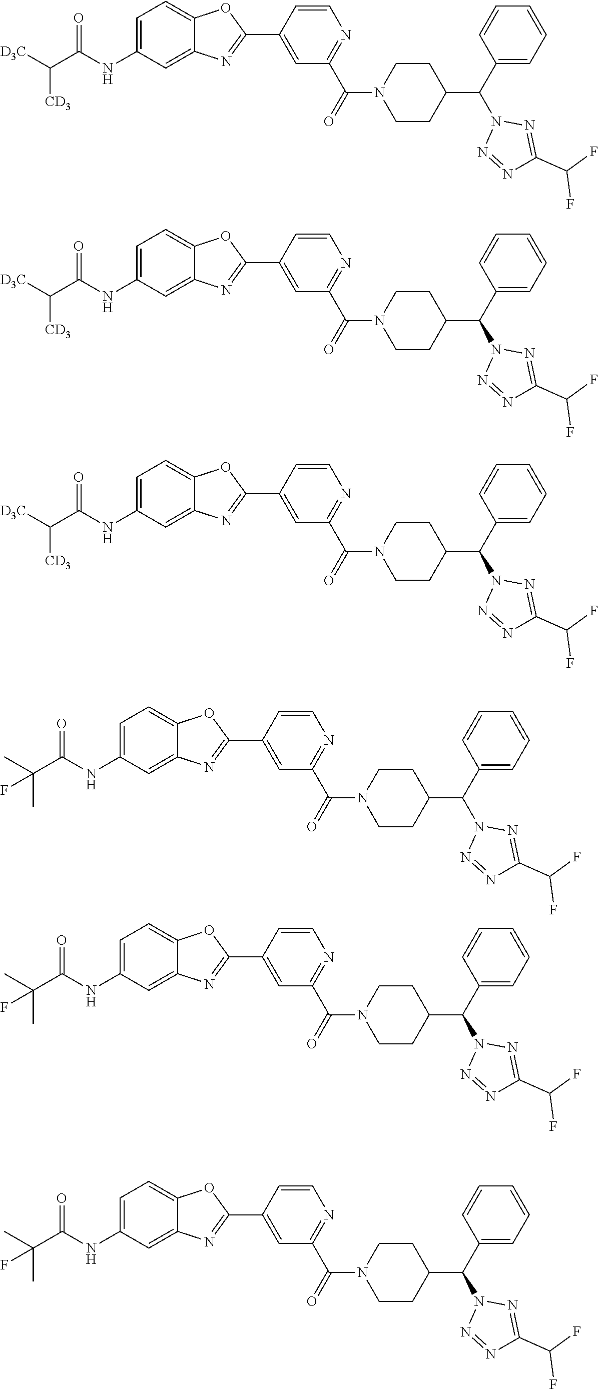

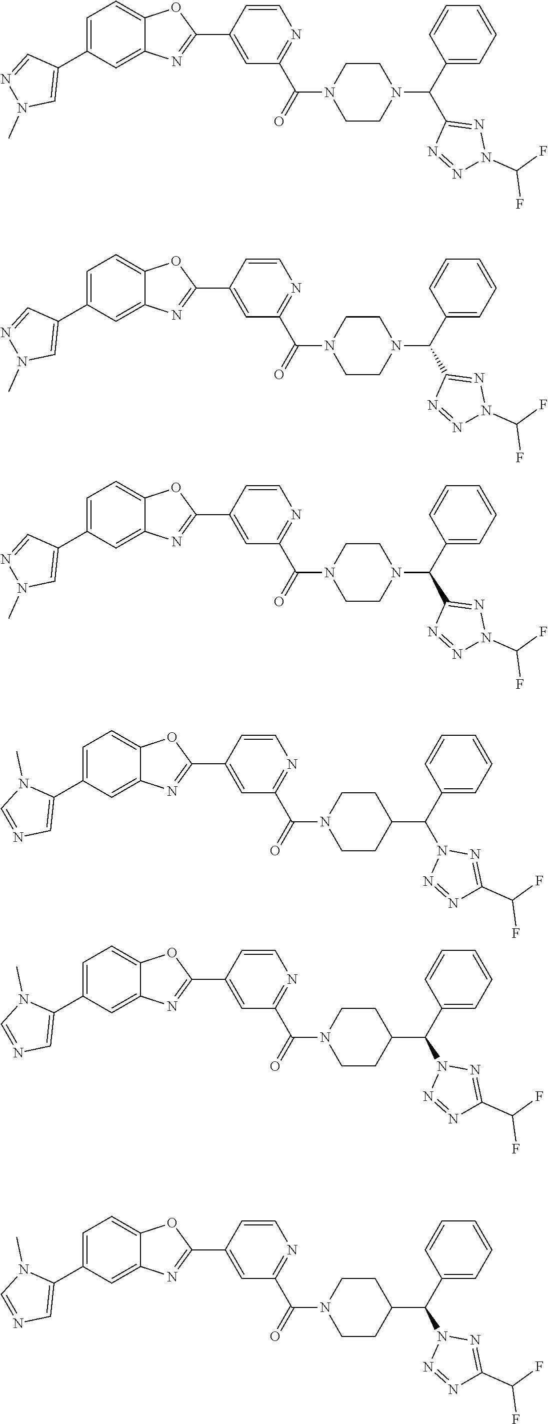

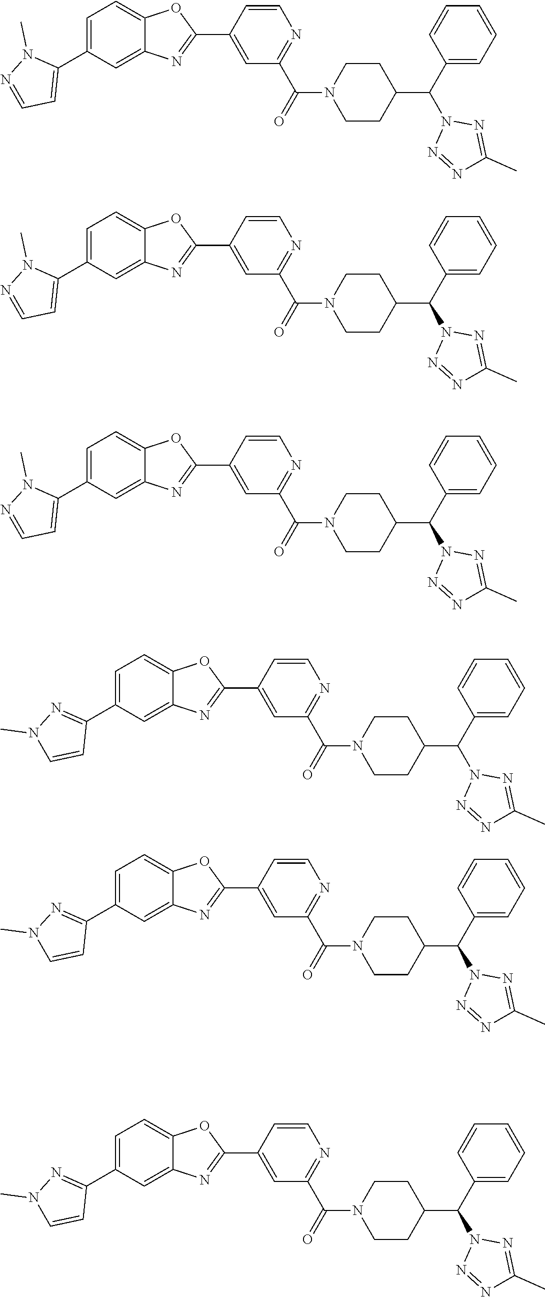

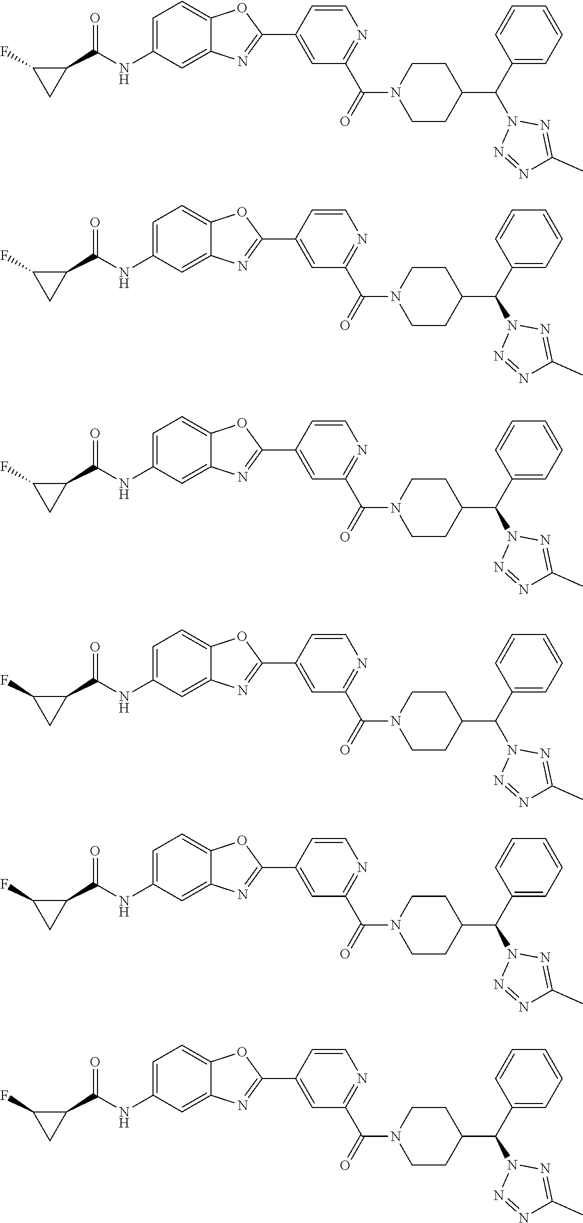

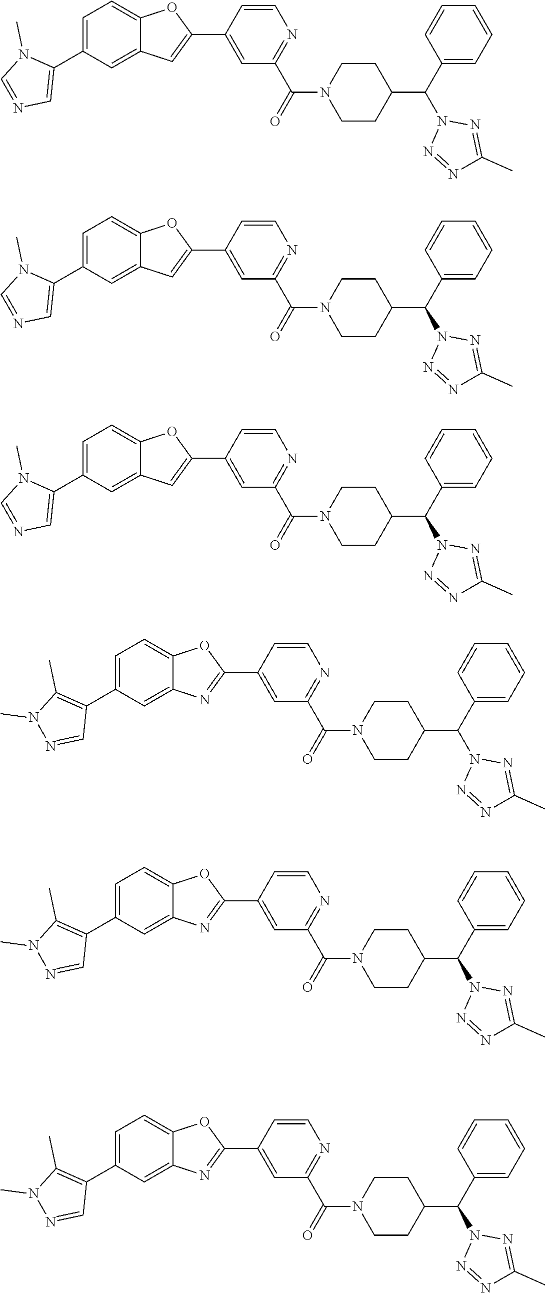

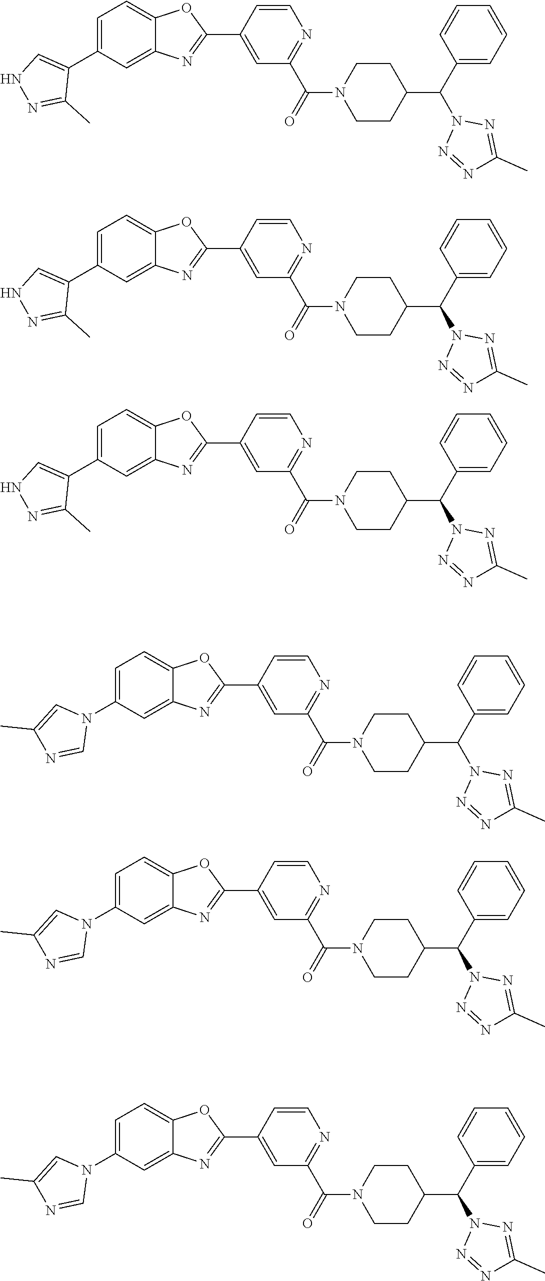

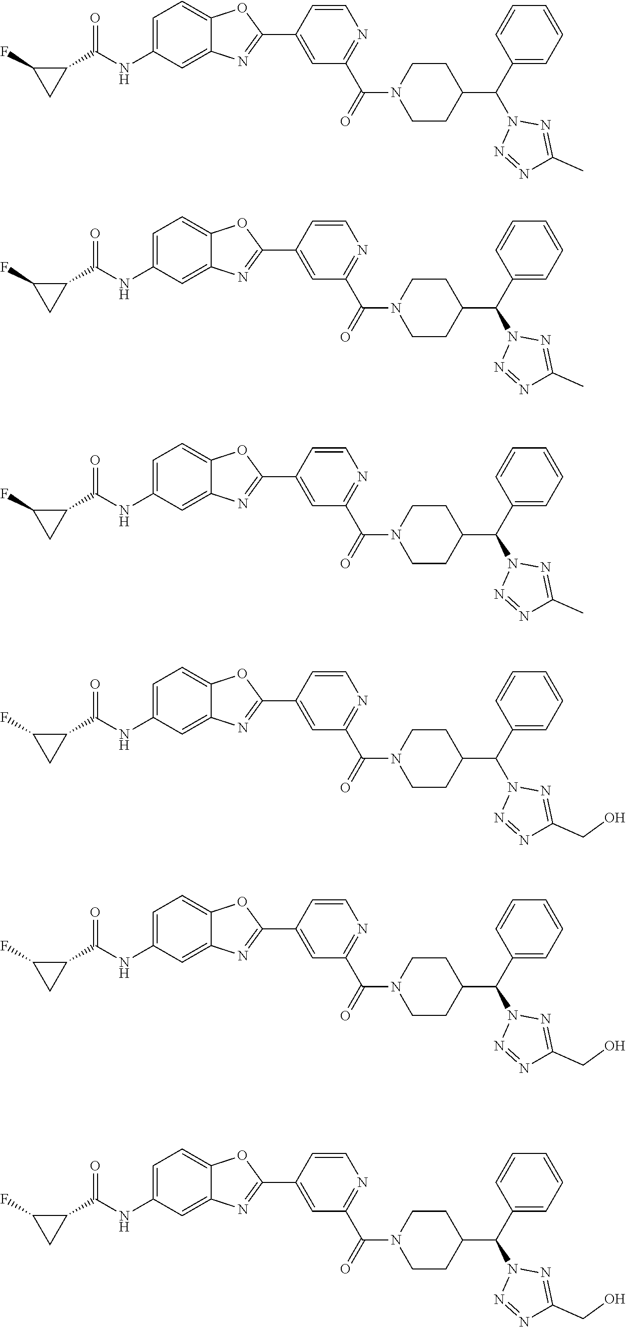

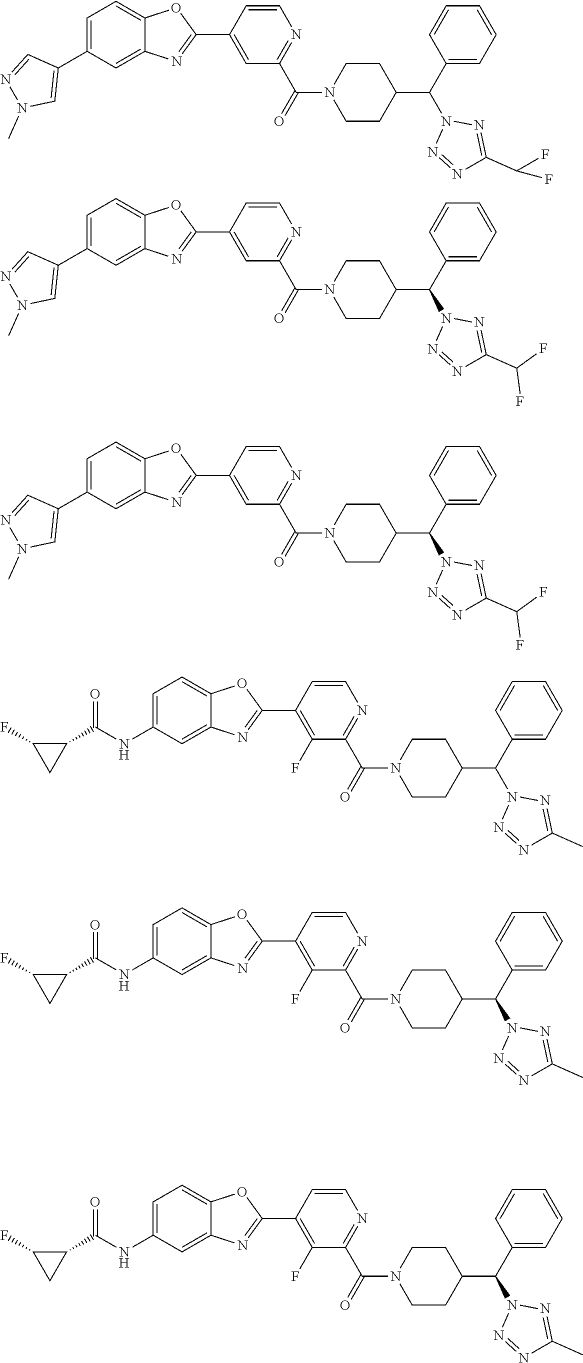

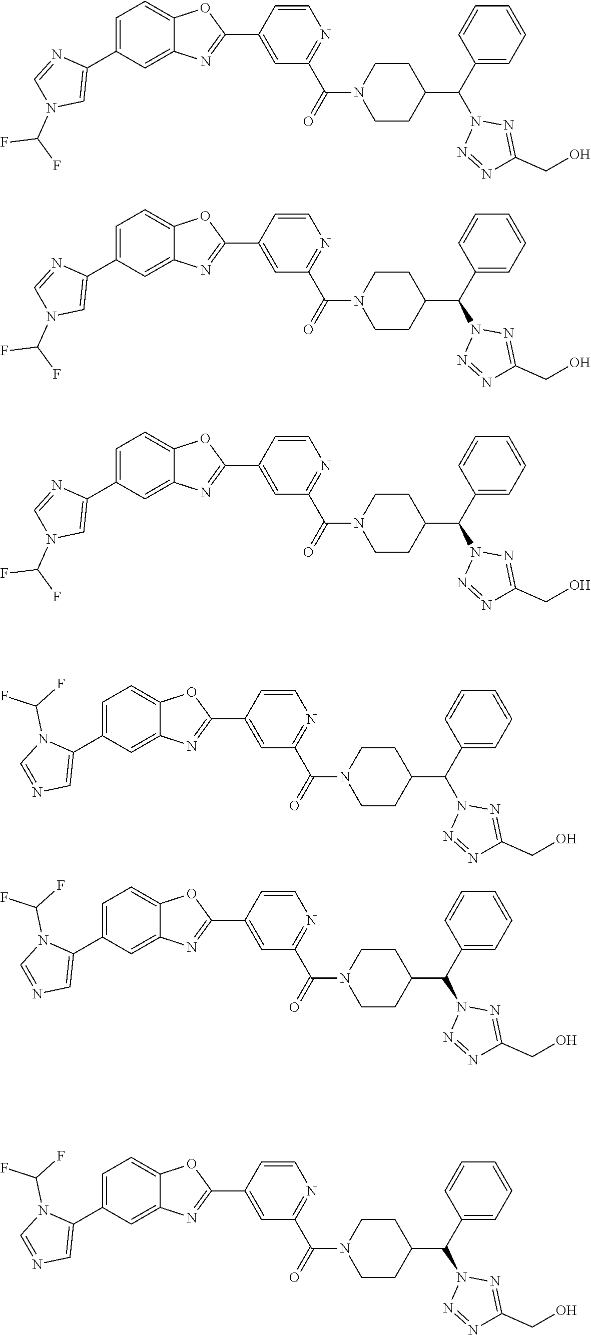

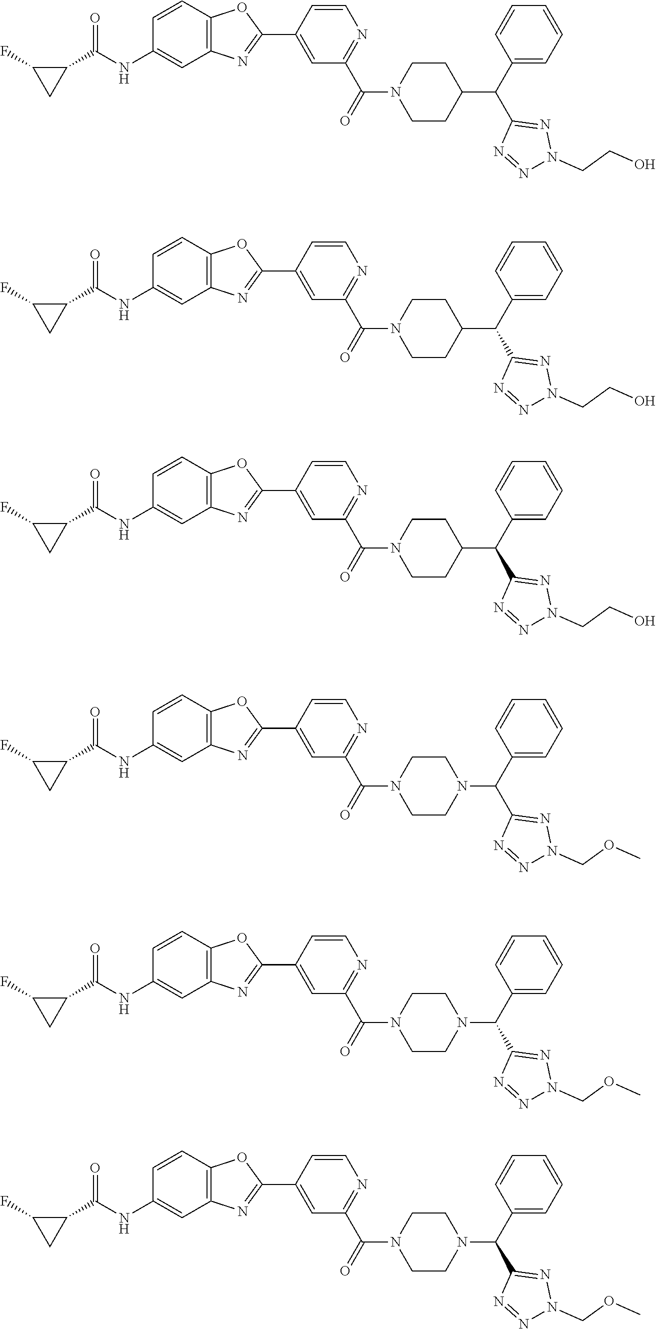

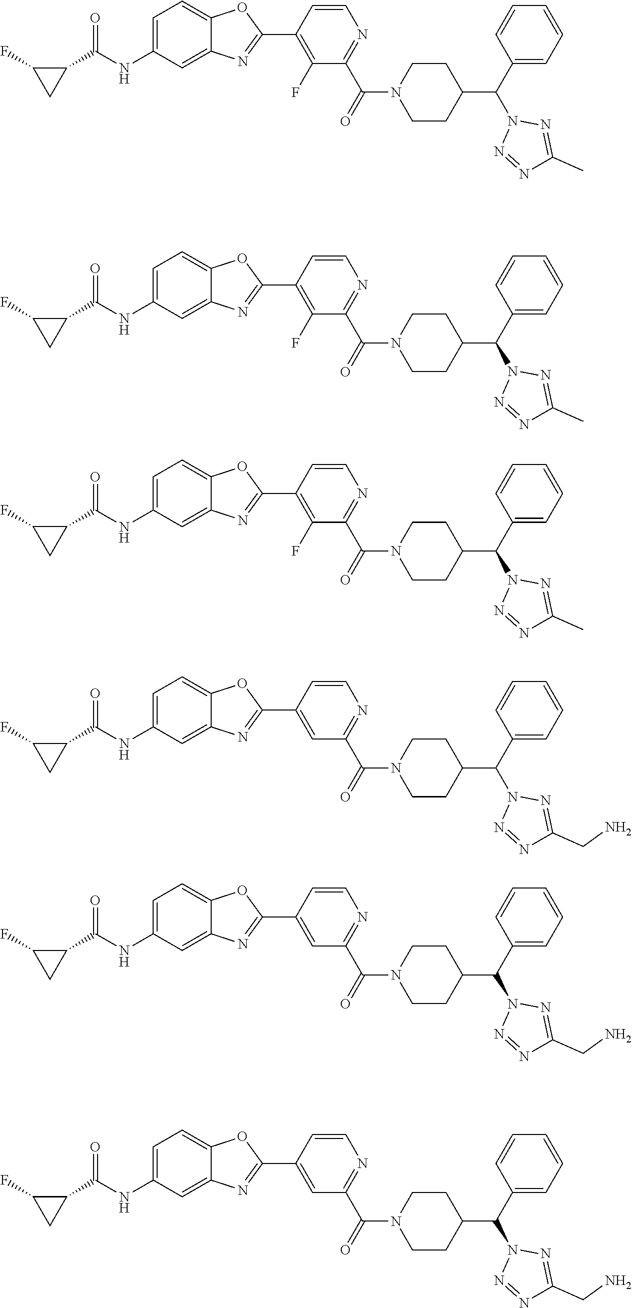

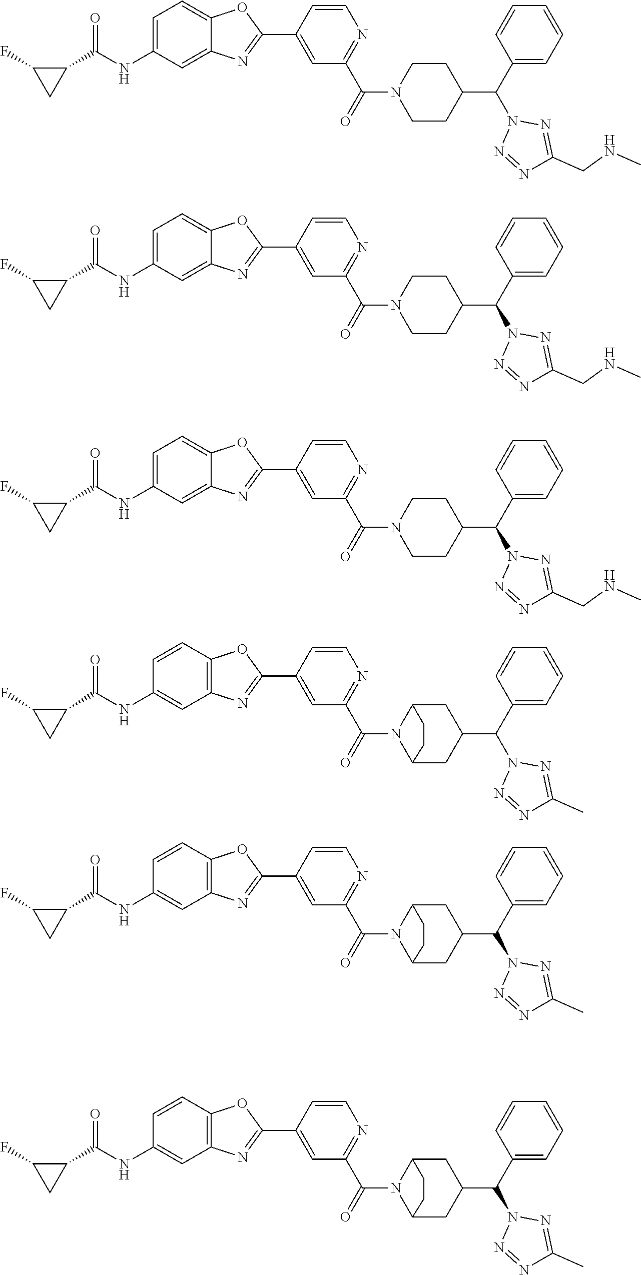

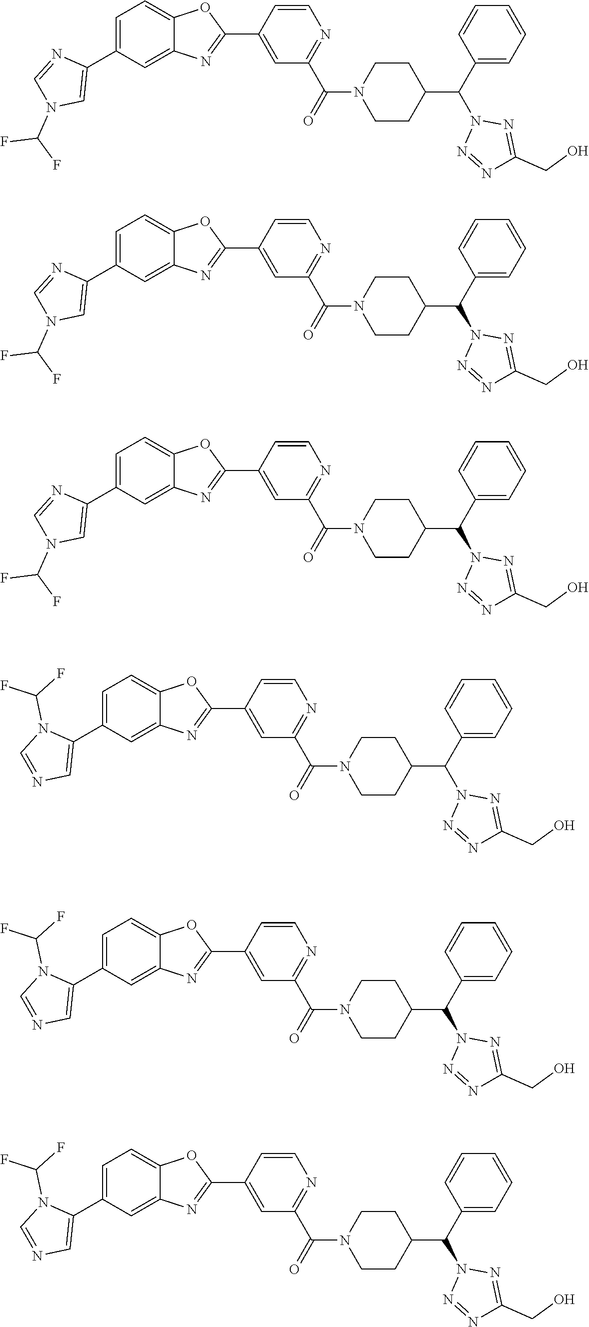

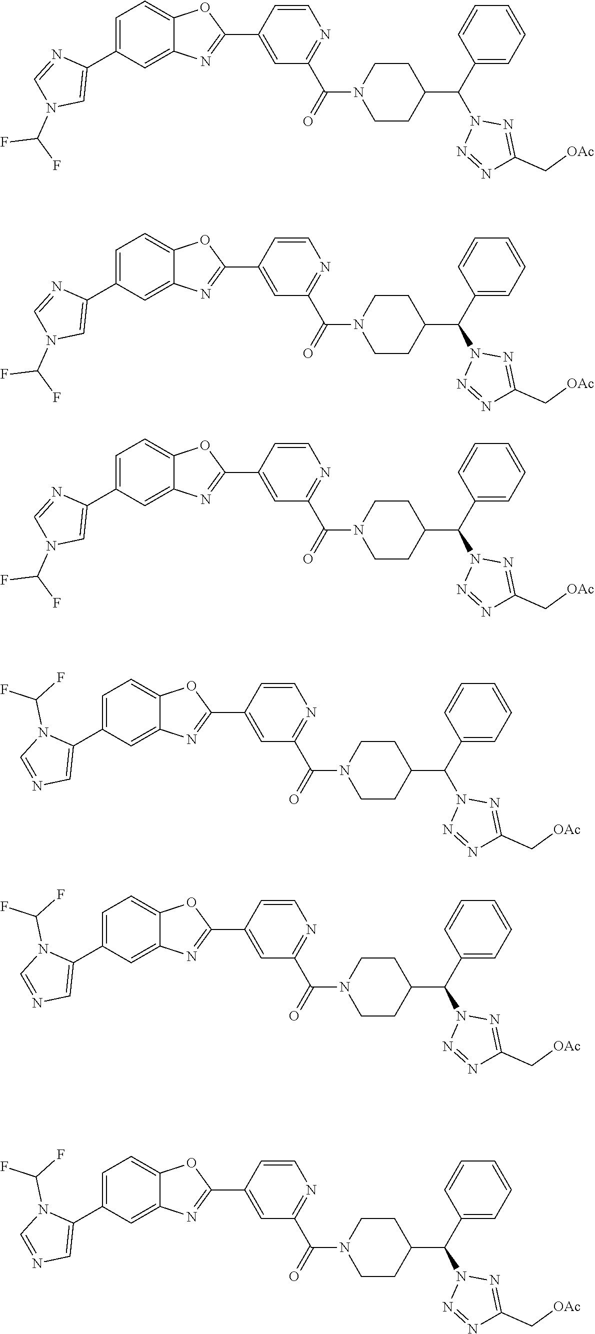

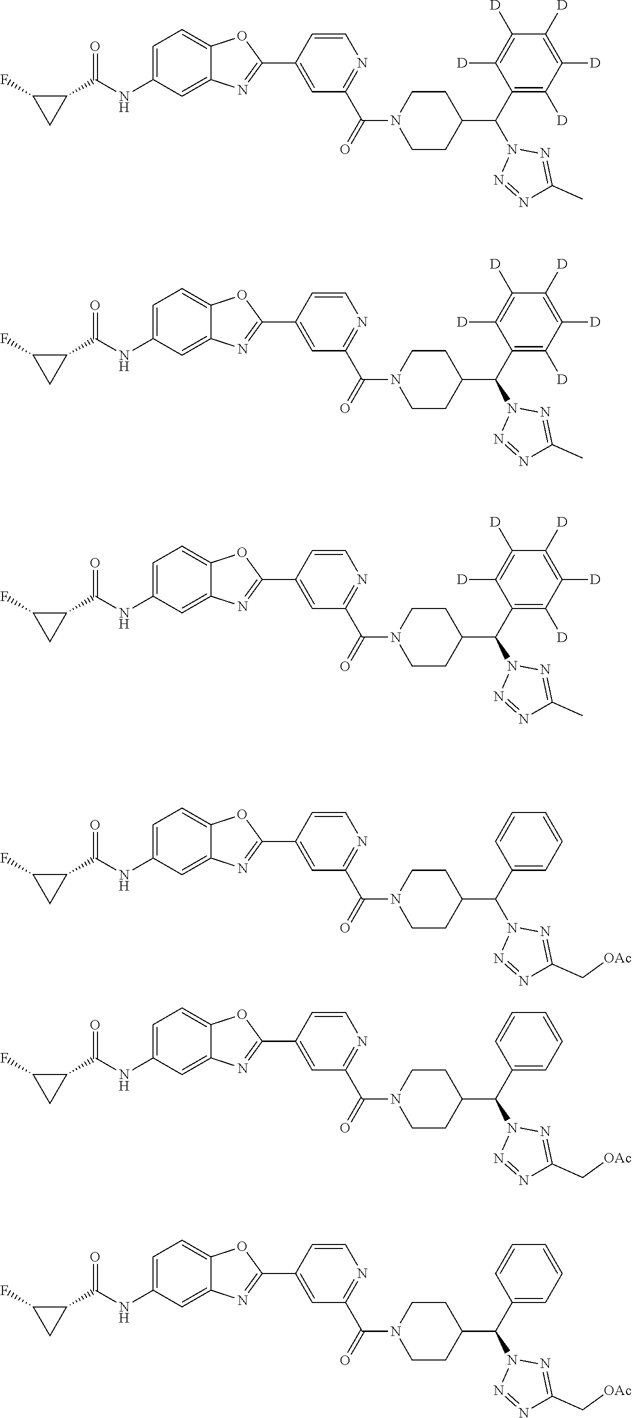

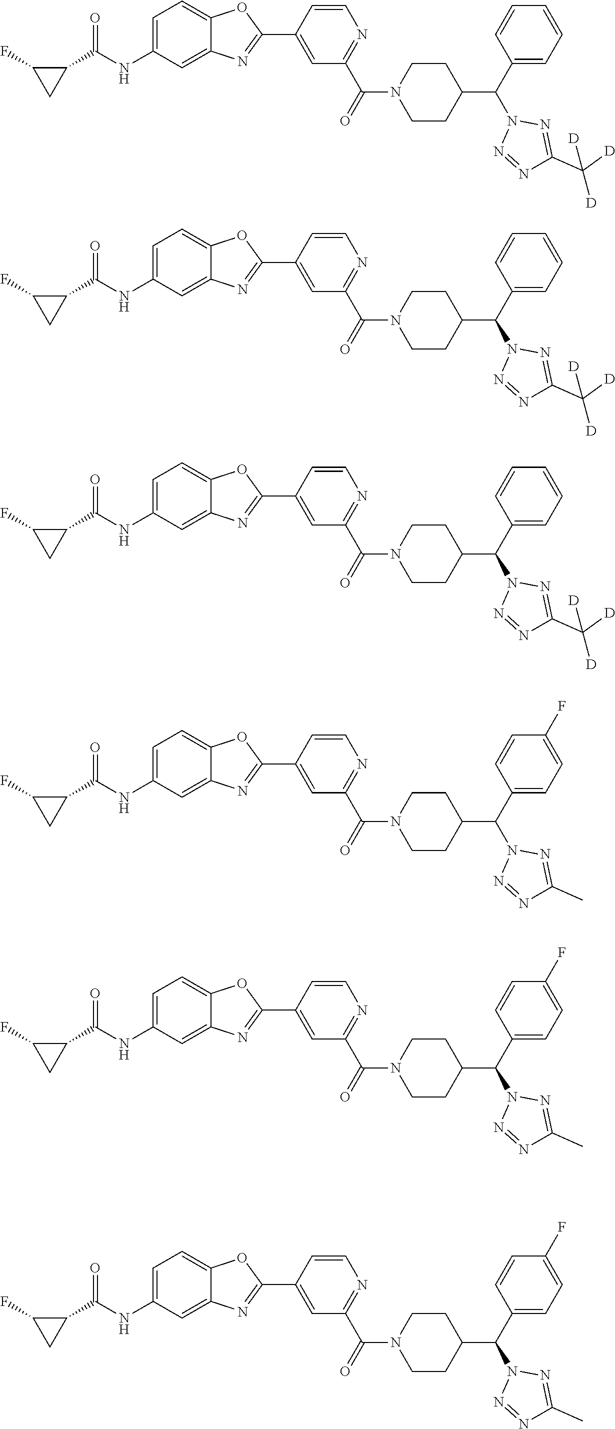

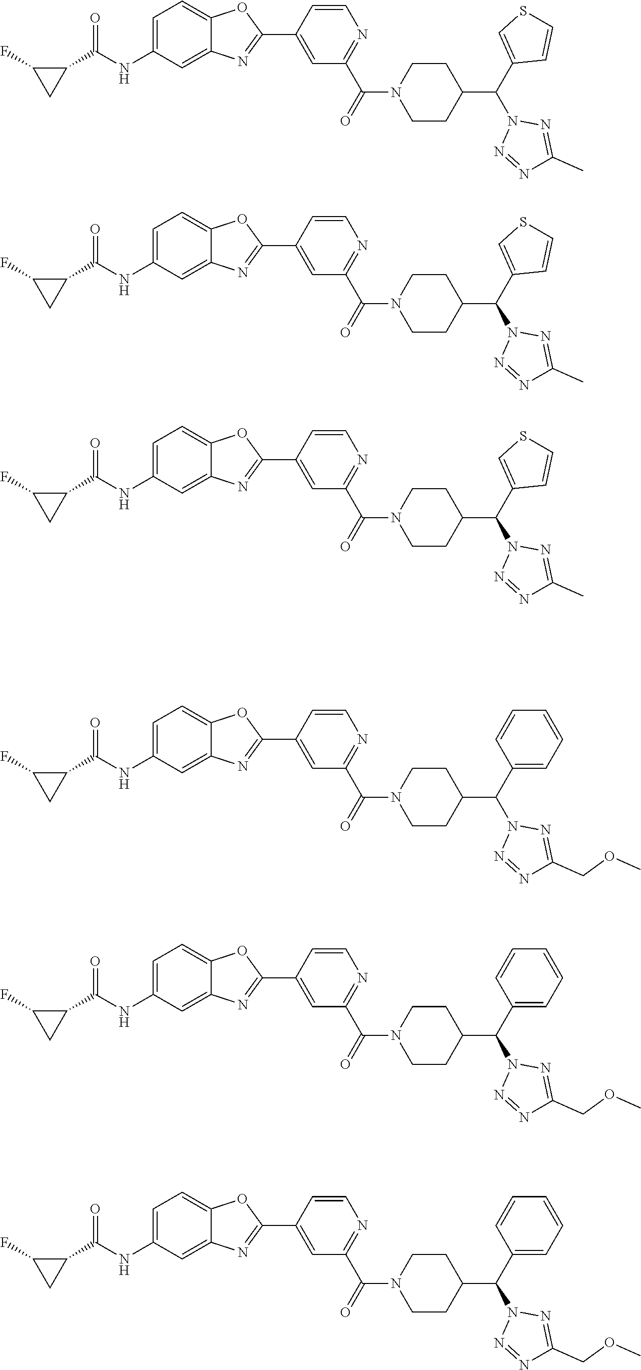

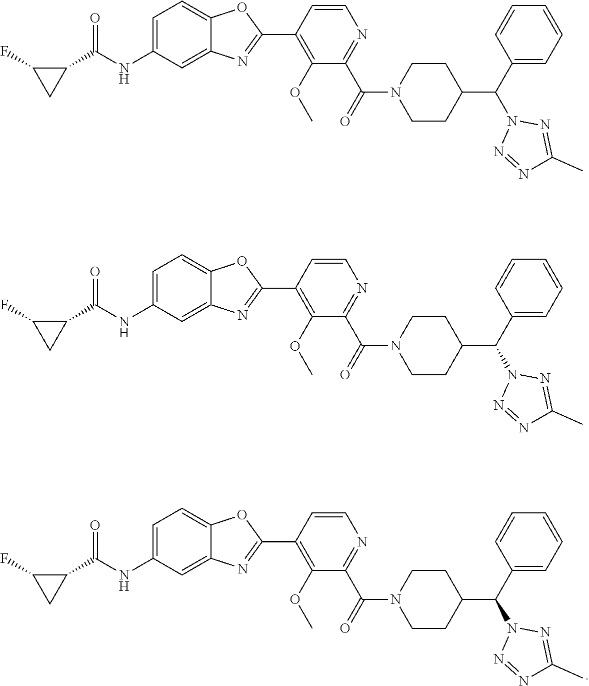 [SMILES: COc1c(-c2nc3cc(NC(=O)[C@@H]4C[C@@H]4F)ccc3o2)ccnc1C(=O)N1CCC(C(c2ccccc2)n2nnc(C)n2)CC1 ; COc1c(-c2nc3cc(NC(=O)[C@@H]4C[C@@H]4F)ccc3o2)ccnc1C(=O)N1CCC([C@H](c2ccccc2)n2nnc(C)n2)CC1 ; COc1c(-c2nc3cc(NC(=O)[C@@H]4C[C@@H]4F)ccc3o2)ccnc1C(=O)N1CCC([C@@H](c2ccccc2)n2nnc(C)n2)CC1]

Another object of the present disclosure is to provide a pharmaceutical composition, wherein the pharmaceutical composition contains a therapeutically effective amount of the above-mentioned compound, the isomer thereof or the pharmaceutically acceptable salt thereof, and a pharmaceutically acceptable carrier and excipient.

Another object of the present disclosure is to provide a pharmaceutical composition, which contains a therapeutically effective amount of the above-mentioned compound, the isomer thereof or the pharmaceutically acceptable salt thereof, and a pharmaceutically acceptable carrier and excipient.

Another object of the present disclosure is to provide the use of the above-mentioned compound, the isomer thereof or the pharmaceutically acceptable salt thereof and the composition thereof in the preparation of an anti-influenza virus medicament.

The compound of the present disclosure exhibits excellent anti-influenza virus effects and actions and has the advantages of improving oral bioavailability, reducing toxic side effects, improving safety, extending half-life, extending action time, etc.

The compound of the present disclosure can be synthesized according to the following routes:

### Synthetic Method I:

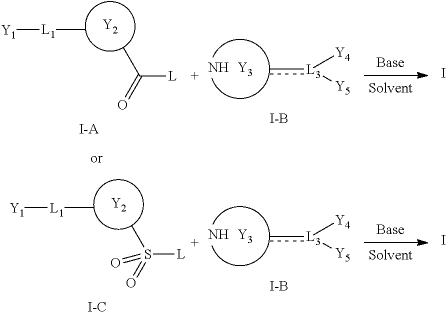

a compound of general formula (I) can be obtained by a condensation reaction of a compound of general formula I-A or I-C with a compound of general formula I-B under the conditions of a suitable base and solvent, wherein L represents a suitable leaving group;
and the definitions of Y₁, L₁, Y₂, Y₃, L₃, Y₄ and Y₅ are consistent with those of general formula (I);

### Synthetic Method II:

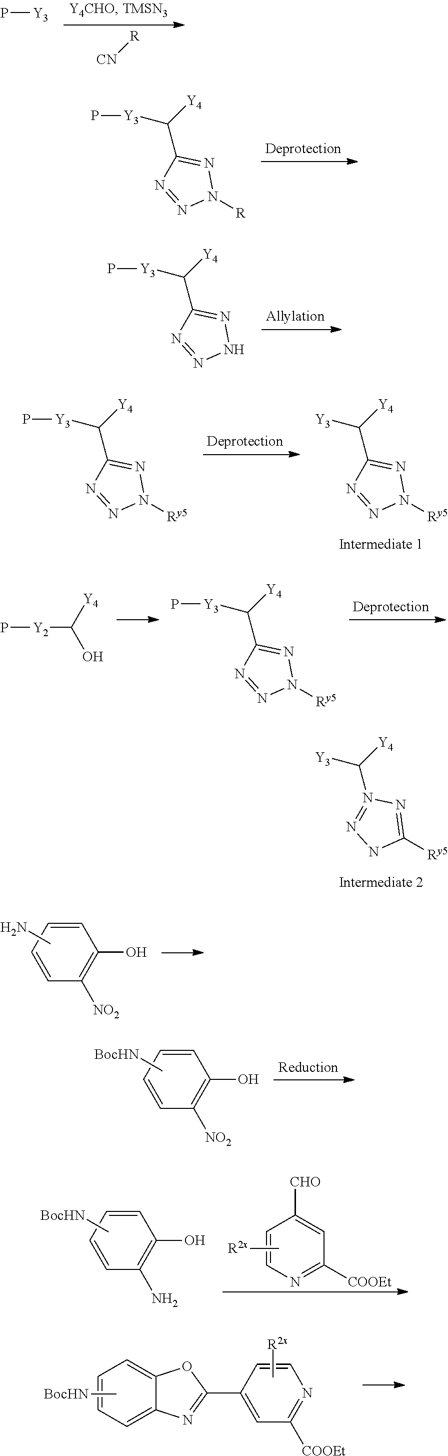

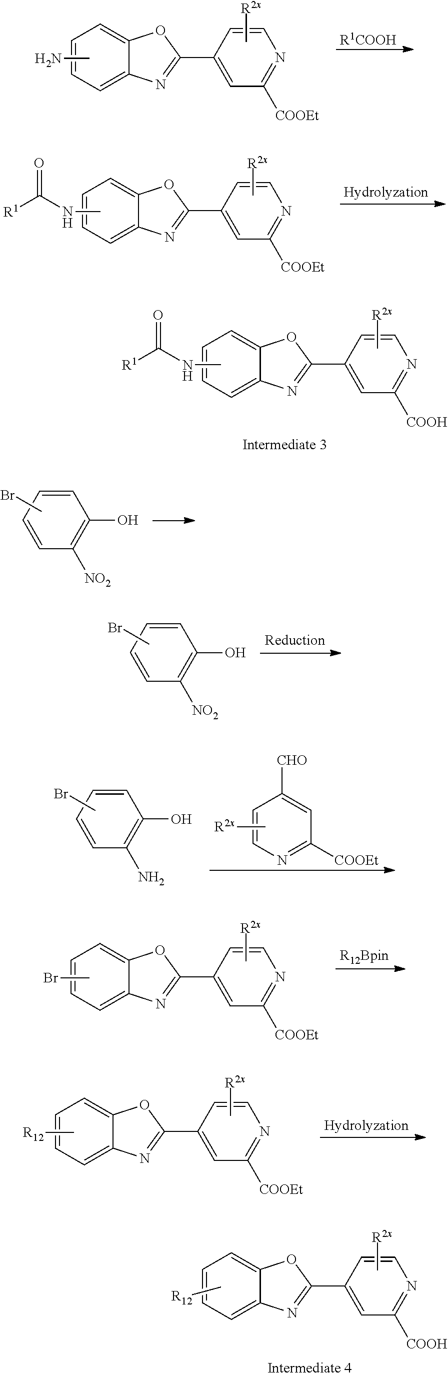

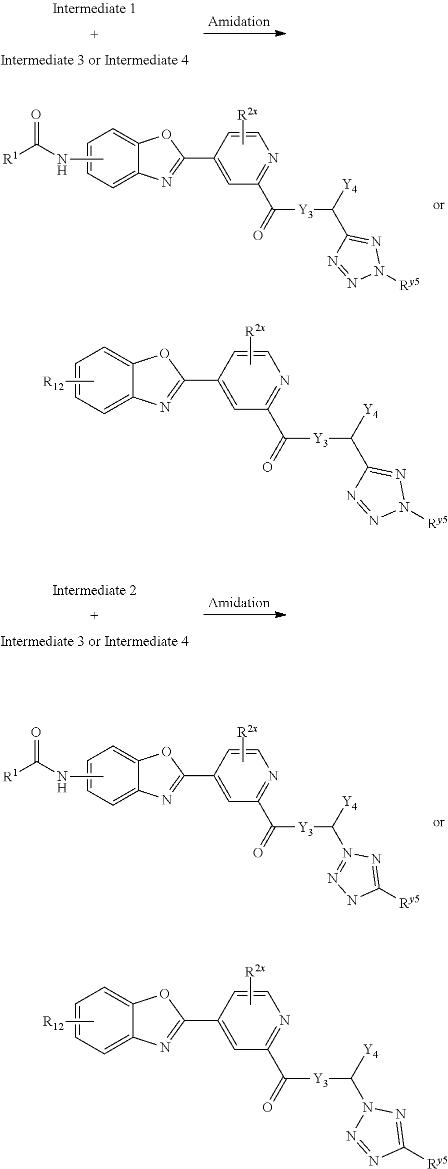

wherein P represents a leaving group, R^{2x} is a substituent of Y₂, and the definitions of Y₂, Y₃ and Y₄ are consistent with those of general formula (I);

### Synthetic Method III:

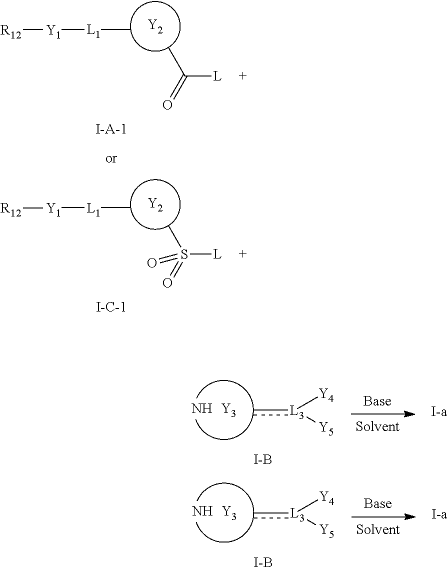

a compound of general formula (I-a) can be obtained by a condensation reaction of a compound of general formula I-A-1 or I-C-1 with a compound of general formula I-B under the conditions of a suitable base and solvent, wherein
the definitions of R₁₂, Y₁, L₁, Y₂, Y₃, L₃, Y₄ and Y₅ are consistent with those of general formula (I-a), and L represents a suitable leaving group.

Unless stated to the contrary, the terms used in the present disclosure have the following meanings.

The carbon, hydrogen, oxygen, sulfur, nitrogen or F, Cl, Br, I involved in the groups and compounds of the present disclosure all comprises their isotopes, and the carbon, hydrogen, oxygen, sulfur or nitrogen involved in the groups and compounds of the present disclosure is optionally further substituted with one or more of their corresponding isotopes, wherein the isotopes of carbon comprise ¹²C, ¹³C and ¹⁴C, the isotopes of hydrogen comprise protium (H), deuterium (D, also known as heavy hydrogen), tritium (T, also known as superheavy hydrogen), the isotopes of oxygen comprise ¹⁶O, ¹⁷O and ¹⁸O, the isotopes of sulfur comprise ³²S, ³³S, ³⁴S and ³⁶S, the isotopes of nitrogen comprise ¹⁴N and ¹⁵N, the isotopes of fluorine comprise ¹⁷F and ¹⁹F, the isotopes of chlorine comprise ³⁵Cl and ³⁷Cl, and the isotopes of bromine comprise ⁷⁹Br and ⁸¹Br.

Prefix “Cx-y” (wherein x and y are integers) used herein refers to the number of carbon atoms in an indicated group. Therefore, C₁₋₈ alkyl contains 1-8 carbon atoms, C₃₋₈ cycloalkyl contains 3-8 carbon atoms and C₁₋₈ alkoxy contain 1-8 carbon atoms.

The term “alkyl” refers to a straight or branched saturated aliphatic hydrocarbon group containing 1 to 20 carbon atoms, preferably alkyl containing 1 to 8 carbon atoms, more preferably alkyl containing 1 to 6 carbon atoms, and further preferably alkyl containing 1 to 4 carbon atoms. Non-limiting examples include methyl, ethyl, n-propyl, isopropyl, n-butyl, sec-butyl, neobutyl, tert-butyl, n-pentyl, isoamyl, neopentyl, n-hexyl and various branched isomers thereof, the alkyl can be optionally further substituted with 0 to 6 substituents selected from F, Cl, Br, I, hydroxyl, sulfhydryl, nitro, cyano, amino, alkylamino, acylamino, alkenyl, alkynyl, C₁₋₆ alkyl, C₁₋₆ hydroxyalkyl, C₁₋₆ alkoxy, 3 to 8 membered carbocyclyl, 3 to 8 membered heterocyclyl, 3 to 8 membered carbocyclyloxy, 3 to 8 membered heterocyclyloxy, carboxyl or a carboxylate group, and the definition of the alkyl described herein is consistent with this definition.

The term “alkylene” refers to a straight or branched chain divalent saturated hydrocarbon group, including —(CH₂)_{y}— (y is an integer from 1 to 10), and examples of alkylene include, but are not limited to, methylene, ethylene, propylene, butylene, etc.; the alkylene can be optionally further substituted with 0-5 substituents selected from F, Cl, Br, I, ═O, —CH₂F, —CHF₂, —CF₃, —OCH₂F, —OCHF₂, —OCF₃, hydroxyl, nitro, cyano, isocyano, alkyl, hydroxylalkyl, alkoxy, carbocyclyl, heterocyclyl, C₂₋₈ alkenyl and C₂₋₈ alkynyl. When the number of substituents in alkylene is 2 or more, the substituents can be fused together to form a cyclic structure. The “alkylene” herein is as defined above.

The “alkoxy” refers to —O-alkyl. Non-limiting examples include methoxy, ethoxy, n-propoxy, isopropoxy, n-butoxy, sec-butoxy, tert-butoxy, n-pentoxy, n-hexyloxy, cyclopropoxy and cyclobutoxy. The alkyl can be optionally further substituted with 0 to 5 substituents selected from F, Cl, Br, I, hydroxyl, sulfhydryl, nitro, cyano, amino, alkylamino, alkenyl, alkynyl, alkyl, hydroxyalkyl, alkoxy, carbocyclyl, heterocyclyl, carbocyclyloxy, heterocyclyloxy, carboxyl or a carboxylate group. The definition of the alkoxy described herein is consistent with this definition.

The term “alkenyl” refers to a straight or branched chain monovalent unsaturated hydrocarbon group having at least 1, usually 1, 2 or 3 carbon-carbon double bonds, with a main chain comprising 2 to 10 carbon atoms, further preferably 2 to 6 carbon atoms, and more preferably 2 to 4 carbon atoms. Examples of alkenyl include, but are not limited to vinyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene, 1,4-hexadiene, etc.; the alkenyl can be optionally further substituted with 0-5 substituents selected from F, Cl, Br, I, ═O, —CH₂F, —CHF₂, —CF₃, —OCH₂F, —OCHF₂, —OCF₃, hydroxyl, nitro, cyano, isocyano, alkyl, hydroxylalkyl, alkoxy, carbocyclyl, heterocyclyl, C₂₋₈ alkenyl and C₂₋₈ alkynyl. The “alkenyl” herein is as defined above.

The term “alkynyl” refers to a straight or branched chain monovalent unsaturated hydrocarbon group having at least 1, usually 1, 2 or 3 carbon-carbon triple bonds, with a main chain comprising 2 to 10 carbon atoms, further preferably 2 to 6 carbon atoms, and more preferably 2 to 4 carbon atoms. Examples of alkynyl include, but are not limited to ethynyl, 1-propynyl, 2-propynyl, butynyl, 2-butynyl, 3-butynyl, 1-methyl-2-propynyl, 4-pentynyl, 3-pentynyl, 1-methyl-2-butynyl, 2-hexynyl, 3-hexynyl, 2-heptynyl, 3-heptynyl, 4-heptynyl, 3-octynyl, 3-nonynyl, 4-decynyl, etc.; the alkynyl can be optionally further substituted with 0-5 substituents selected from F, Cl, Br, I, ═O, —CH₂F, —CHF₂, —CF₃, —OCH₂F, —OCHF₂, —OCF₃, hydroxyl, nitro, cyano, isocyano, alkyl, hydroxylalkyl, alkoxy, carbocyclyl, heterocyclyl, C₂₋₈ alkenyl and C₂₋₈ alkynyl. The “alkynyl” herein is as defined above.

The term “cycloalkyl” refers to a hydrocarbon ring of substituted or unsubstituted, saturated, partially unsaturated or fully unsaturated non-aromatic ring. Cycloalkyl can be a monocyclic, bicyclic or polycyclic ring. The bicyclic or polycyclic ring can be a fused ring, a spiro ring or a bridged ring. Unless otherwise specified, cycloalkyl usually contains 3 to 20 carbon atoms; when cycloalkyl is monocyclic cycloalkyl, the cycloalkyl contains preferably 3-15 carbon atoms, preferably 3-10 carbon atoms, also preferably 3-8 carbon atoms, more preferably 3-6 carbon atoms, and further preferably 3-4 carbon atoms; when cycloalkyl is bicyclic or polycyclic cycloalkyl, the cycloalkyl contains preferably 4-12 carbon atoms, preferably 4-11 carbon atoms, also preferably 5-11 carbon atoms, more preferably 6-11 carbon atoms, and further preferably 6-10 carbon atoms; and non-limiting examples include cyclopropyl, cyclobutyl, cyclopentyl, cyclohexyl, cycloheptyl, butenyl, cyclopentenyl, cyclohexenyl,

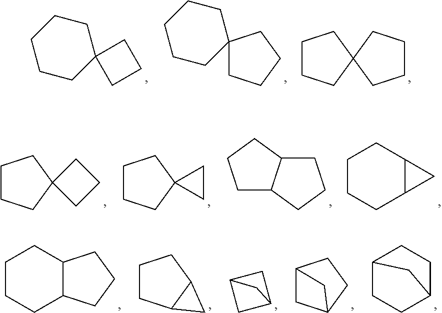

etc.

The term “heterocycloalkyl” refers to a ring of substituted or unsubstituted, saturated, partially unsaturated or fully unsaturated non-aromatic ring containing at least one heteroatom. Unless otherwise specified, heterocycloalkyl is a 3 to 20 membered ring. When heterocycloalkyl is monocyclic heterocycloalkyl, the heterocycloalkyl is preferably 3 to 15 membered, preferably 3-10 membered, also preferably 3-8 membered, and further preferably 3-6 membered ring; when heterocycloalkyl is bicyclic or polycyclic heterocycloalkyl, the heterocycloalkyl is preferably 4-12 membered, preferably 4-11 membered, also preferably 5-11 membered, more preferably 6-11 membered, and further preferably 6-10 membered ring; heterocycloalkyl can be a monocyclic, bicyclic or polycyclic ring, and the bicyclic or polycyclic ring can be a bridged ring, a fused ring and a spiro ring, in which the heteroatoms are selected from N, S, O, P and Si heteroatoms and an oxidation state thereof; when heterocycloalkyl is a bicyclic or polycyclic ring, at least one ring contains at least one heteroatom, and the heterocycloalkyl can be a bicyclic or polycyclic ring formed by a ring containing the heteroatom(s) and a ring containing no heteroatom; when heterocycloalkyl is connected to other groups, a connection point can be at a heteroatom or a carbon atom; and non-limiting examples include azacyclobutyl, morpholinyl, piperazinyl, piperidyl, tetrahydropyranyl, oxacyclobutyl, pyranyl, azacyclopentenyl, azacyclohexenyl, oxacyclopentenyl, oxacyclohexenyl, etc.

The term “carbocyclyl” or “carbocyclic ring” refers to a saturated or unsaturated non-aromatic monocyclic or polycyclic group, and the number of carbon atoms is 3-12, preferably 3-8. The polycyclic ring is formed by the sharing of one or two atoms between carbocyclyl and carbocyclyl. Non-limiting example includes cyclopropyl, cyclobutyl, cyclopentyl, 1-cyclopentyl-1-alkenyl, 1-cyclopentyl-2-alkenyl, 1-cyclopentyl-3-alkenyl, cyclohexyl, 1-cyclohexyl-2-alkenyl, 1-cyclohexyl-3-alkenyl, cyclohexenyl, cyclohexadienyl, cycloheptyl, cyclooctyl, cyclononyl, cyclodecyl,

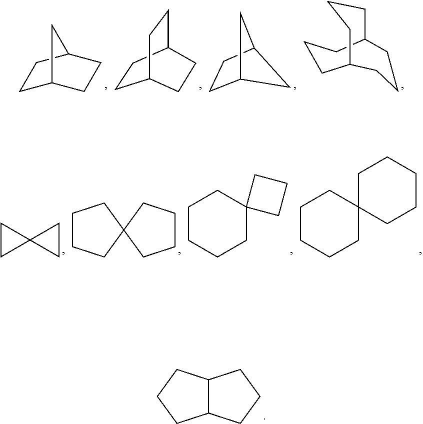

The carbocyclyl can be optionally further substituted with 0-5 substituents selected from F, Cl, Br, I, ═O, —CH₂F, —CHF₂, —CF₃, —OCH₂F, —OCHF₂, —OCF₃, hydroxyl, nitro, cyano, isocyano, alkyl, hydroxylalkyl, alkoxy, carbocyclyl, heterocyclyl, C₂₋₈ alkenyl and C₂₋₈ alkynyl. The “carbocyclyl” herein is as defined above.

The term “heterocyclyl” or “heterocyclic ring” represents a non-aromatic monocyclic or polycyclic group having a ring atom number of 3-12 and containing one or more heteroatoms (such as N, O and S), wherein the number of heteroatoms is 1-4. The polycyclic ring is formed by the sharing of one or two atoms between heterocyclyl and heterocyclyl or between heterocyclyl and carbocyclyl. Heterocyclyl can be connected to a heteroatom or a carbon atom. Non-limiting examples include epoxyethyl, epoxypropyl, azacyclopropyl, oxacyclobutyl, azacyclobutyl, thiacyclobutyl, 1,3-dioxolanyl, 1,4-dioxolanyl, 1,3-dioxanyl, azacycloheptyl, oxacycloheptyl, thiepanyl, dihydrofuryl, dihydropyranyl, tetrahydrofuryl, tetrahydrothienyl, tetrahydropyranyl, tetrahydrothiopyranyl, tetrahydropyrrolyl, tetrahydro imidazolyl, tetrahydrothiazolyl, tetrahydropyranyl, 2-pyrrolinyl, 3-pyrrolinyl, 2H-pyranyl, 4H-pyranyl, dioxacyclohexyl, 1,3-dioxolanyl, dithianyl, dithiolanyl, dihydrothienyl, pyrazolidinyl, imidazolinyl, imidazolidinyl, pyrrolocyclohexyl,

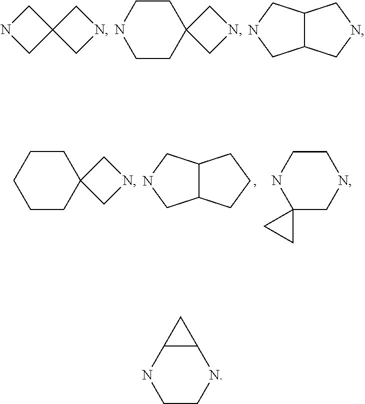

The heterocyclyl can be optionally further substituted with 0-5 substituents selected from F, Cl, Br, I, ═O, —CH₂F, —CHF₂, —CF₃, —OCH₂F, —OCHF₂, —OCF₃, hydroxyl, nitro, cyano, isocyano, alkyl, hydroxylalkyl, alkoxy, carbocyclyl, heterocyclyl, C₂₋₈ alkenyl and C₂₋₈ alkynyl. The “heterocyclyl” herein is as defined above.

The term “aryl” represents an aromatic monocyclic or polycyclic group having a ring atom number of 5-15, preferably 5-10 ring atoms. The polycyclic ring is formed by the sharing of one or two atoms between aryl and aryl, between aryl and heterocyclyl or between aryl and carbocyclyl. Non-limiting examples include phenyl, naphthyl, benzodihydrofuryl, carbocyclyl,

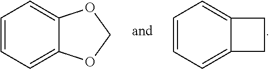 [SMILES: c1ccc2c(c1)OCO2 ; c1ccc2c(c1)CC2]

The term “heteroaryl” represents an aromatic heterocyclic ring, particularly an aromatic monocyclic or polycyclic group having a ring atom number of 5-15, preferably 5-10 and containing one or more heteroatoms (such as N, O and S), wherein the number of heteroatoms is 1-4. The polycyclic ring is formed by the sharing of one or two atoms between heteroaryl and heteroaryl, between heteroaryl and aryl, between heteroaryl and heterocyclyl, or between heteroaryl and carbocyclyl. Non-limiting examples include pyrrole, furan, thiophene, imidazole, oxazole, oxadiazole, oxatriazole, isoxazole, thiazole, thiadiazole, isothiazole, pyrazole, triazole, tetrazole, pyridine, pyrazine, pyridazine, pyrimidine, triazine, benzopyridyl,

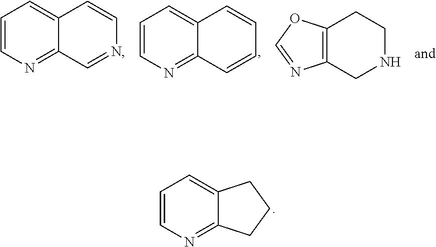

The heteroaryl can be optionally further substituted with 0-5 substituents selected from F, Cl, Br, I, ═O, —CH₂F, —CHF₂, —CF₃, —OCH₂F, —OCHF₂, —OCF₃, hydroxyl, nitro, cyano, isocyano, alkyl, hydroxylalkyl, alkoxy, carbocyclyl, heterocyclyl, C₂₋₈ alkenyl and C₂₋₈ alkynyl. The “heteroaryl” herein is as defined above.

The term “haloalkyl” refers to halogen-substituted alkyl as defined above. Non-limiting examples include fluoromethyl, difluoromethyl, trifluoromethyl, bromomethyl, dibromomethyl, tribromomethyl, fluoroethyl, 2-fluoroethyl-2-yl, 1,1-difluoroethyl-2-yl, 1,2-difluoroethyl-2-yl, 1,1,1-fluoroethyl-2-yl, 1-bromoethyl-2-yl, 2-bromoethyl-2-yl, 1,1,1-tribromoethyl-2-yl, etc.

The term “spiro ring” refers to a 5 to 20 membered polycyclic group sharing one carbon atom (referred to as a spiro atom) between substituted or unsubstituted monocyclic rings, which may contain 0 to 5 double bonds, and may contain 0 to 5 heteroatoms selected from N, O, S, S(═O) and S(═O)₂. Non-limiting examples include:

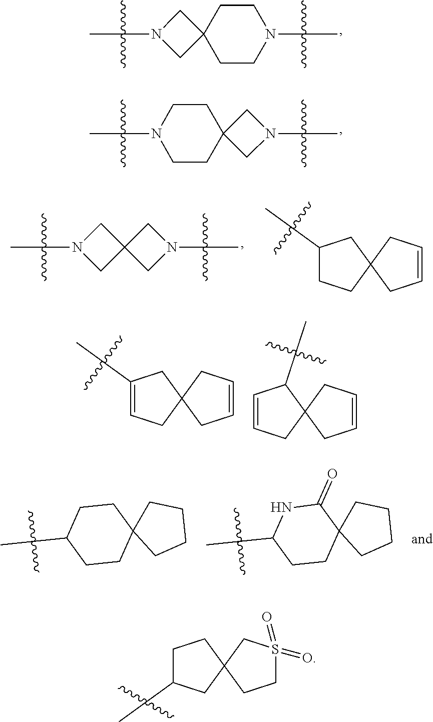

The term “fused ring” refers to a polycyclic group in which each ring in the system shares an adjacent pair of atoms with other rings in the system, wherein one or more of the rings may contain 0 or more double bonds and may be substituted or unsubstituted, and each ring in a fused ring system may contain 0 to 5 heteroatoms selected from N, S, S(═O), S(═O)₂ or O. Non-limiting examples include:

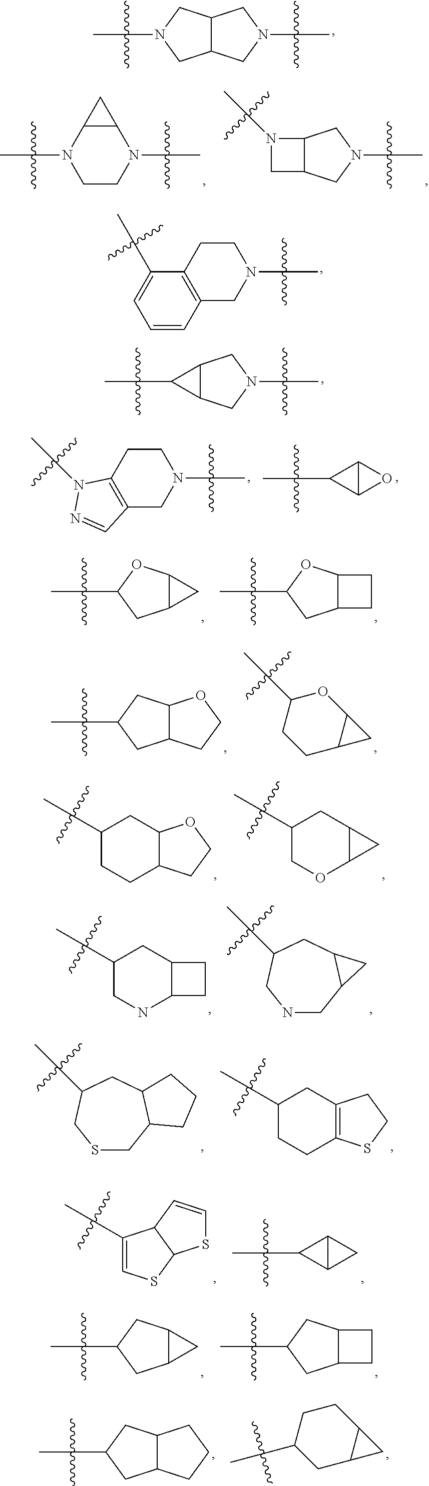

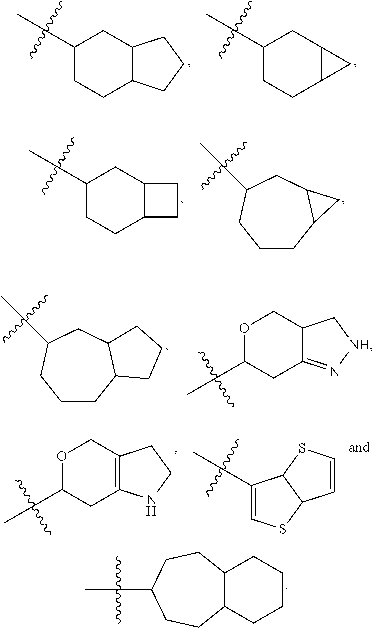

The term “bridged ring” refers to a polycyclic group in which any two rings share two atoms which are not directly connected, wherein the group may contain 0 or more double bonds and can be substituted or unsubstituted, and any ring in a bridged ring system may contain 0 to 5 heteroatoms or groups selected from N, S, S(═O), S(═O)₂ or O. Ring atoms contain 5 to 20 atoms. Non-limiting examples include

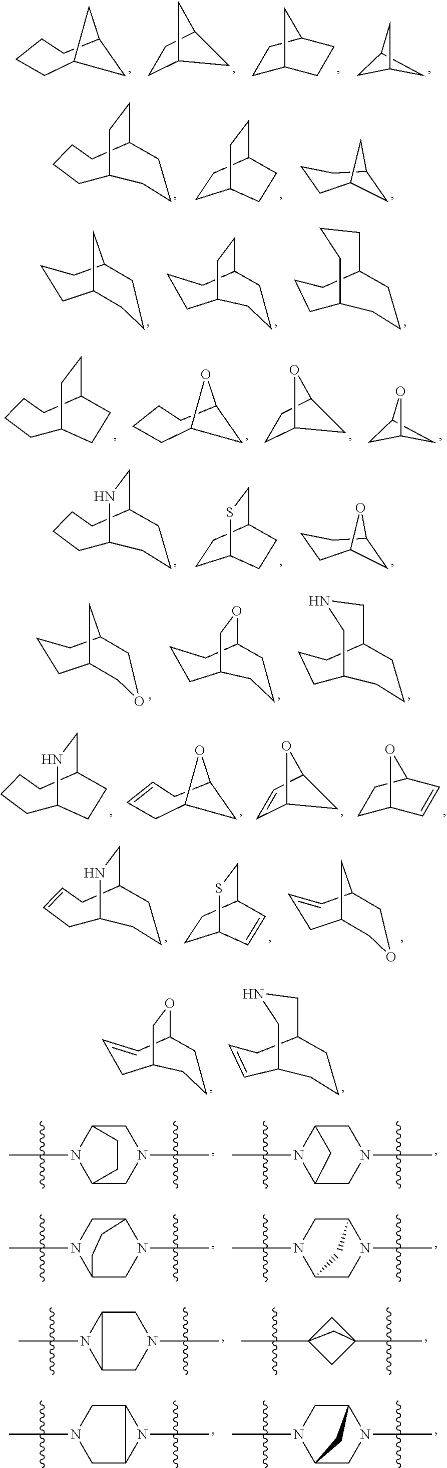

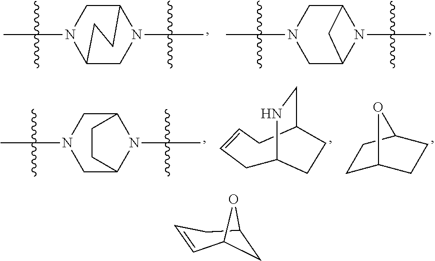

and adamantane.

The term “halogen” refers to fluorine, chlorine, bromine or iodine.

The term “heteroatom” refers to a non-metal element atom other than carbon or hydrogen, particularly a non-metal element atom from groups IVA, VA, and VIA, such as oxygen, sulfur, nitrogen, silicon, and phosphorus. When more than one heteroatom is present, the more than one heteroatom may be the same as each other, or some or all of the heteroatoms may be different from each other.

Unless stated, a connection point of a substituent as used herein can be from any suitable position of the substituent.

The term “pharmaceutically acceptable salt” or “pharmaceutically acceptable salt thereof” refers to a salt maintaining the biological effectiveness and characteristics of a free acid or a free base, and obtained by reacting the free acid with a non-toxic inorganic base or organic base or the free base with a non-toxic inorganic acid or organic acid, and includes alkali metal salt such as sodium salt, potassium salt, and lithium salt; alkaline earth metal salt, such as calcium salt and magnesium salt; other metal salt, such as iron salt, copper salt and cobalt salt; organic base salt, such as ammonium salt, triethylamine salt, pyridine salt, methylpyridine salt, 2,6-dimethylpyridine salt, ethanolamine salt, diethanolamine salt, triethanolamine salt, cyclohexylamine salt, ethylenediamine salt, guanidine salt, isopropylamine salt, trimethylamine salt, tripropylamine salt, triethanolamine salt, diethanolamine salt, ethanolamine salt, dimethyl ethanolamine salt, dicyclohexylamine salt, caffeine salt, procaine salt, choline salt, betaine salt, benethamine penicillin salt, glucosamine salt, N-methylglucosamine salt, theobromine salt, tromethamine salt, purine salt, piperazine salt, morpholine salt, piperidine salt, N-ethylpiperidine salt, tetramethylamine salt, dibenzylamine salt and phenylglycine alkyl ester salt; hydrohalide, such as hydrofluoride, hydrochloride, hydroiodide and hydrobromide; inorganic acid salt, such as hydrochloride, nitrate, sulfate, perchlorate and phosphate; lower alkanesulfonate, such as methanesulfonate, trifluoromethanesulfonate and ethanesulfonate; arylsulfonate, such as benzene sulfonate and p-toluenesulfonate; organic acid salt, such as acetate, benzoate, fumarate, formate, trifluoroacetate, furoate, gluconate, glutamate, glycolate, isethionate, lactate, maleate, malate, mandelate, mucic acid salt, pamoate, pantothenate, stearate, succinate, sulfanilate, tartrate, malonate, 2-hydroxypropionate, citrate, salicylate, oxalate, glycolate, glucuronate, galacturonate, citric acid salt, lysine salt, arginine salt, aspartate and cinnamate.

The term “pharmaceutical composition” represents a mixture of one or more compounds described herein or physiologically/pharmaceutically acceptable salts thereof and other components, wherein the other components comprise physiologically/pharmaceutically acceptable carriers and excipients.

The term “carrier” refers to a carrier or diluent that does not cause significant irritation to the organism and does not eliminate the biological activity and characteristics of the administered compound.

The term “excipient” refers to an inert substance added to a pharmaceutical composition to further depend on the administration of a compound. Examples of excipients include, but not limited to calcium carbonate, calcium phosphate, various sugars and different types of starch, cellulose derivatives (including microcrystalline cellulose), gelatin, vegetable oil, polyethylene glycols, diluents, granulating agents, lubricants, binders, disintegrants, etc.

“Isomer” includes “stereoisomer” and “tautomer”. The term “stereoisomer” refers to an isomer produced by different arrangements of atoms in a molecule in space, including cis-trans isomers, enantiomers and conformational isomers. The term “tautomer” refers to isomers that can be transformed into each other by a reversible chemical reaction called tautomerization, which is a transformation between functional groups usually caused by the accompanying migration of a hydrogen atom and a π bond (a double bond or a triple bond); such as, the following paired compounds: aldehyde/ketone-enol and imine-enamine.

The expression “each independently selected from” refers to that each substituent is independently selected from each other, and each substituent may be the same or different from other substituents.

The expression “optional” or “optionally” refers to that events or circumstances subsequently described may but not necessarily occur, and the description includes the occasions where the events or circumstances occur or do not occur. For example, the expression “alkyl optionally substituted with F” means that the alkyl may but not necessarily be substituted with F, and the description includes the case where the alkyl is substituted with F and the case where the alkyl is not substituted with F.

The term “treatment”. “treating” or “treat” refers to alleviating symptoms associated with diseases, disorders, or conditions, or stopping further development or deterioration of the symptoms.

### DETAILED DESCRIPTION OF EMBODIMENTS

The technical solutions of the present disclosure will be described in detail below in conjunction with examples, but the protection scope of the present disclosure includes but is not limited thereto.

The following examples enable a better understanding of the present disclosure, and do not limit the scope of the present disclosure in any way. The following intermediates and the raw materials and reagents used in the examples are all commercially available.

The abbreviations and the corresponding full names are as follows:
DDQ: 2,3-dichloro-5,6-dicyano-p-benzoquinone
Pd(dppf)Cl₂: 1,1′-bis(diphenylphosphino)ferrocene palladium dichloride
DMF: N,N-dimethylformamide
HATU: 2-(7-azobenzotriazole)-N,N,N′,N′-tetramethyluronium hexafluorophosphate
DIPEA: N,N-diisopropylethylamine
TMSCN: trimethylsilyl cyanide

Intermediate 1:

### 4-(5-acetamidobenzo[d]oxazol-2-yl)picolinic Acid

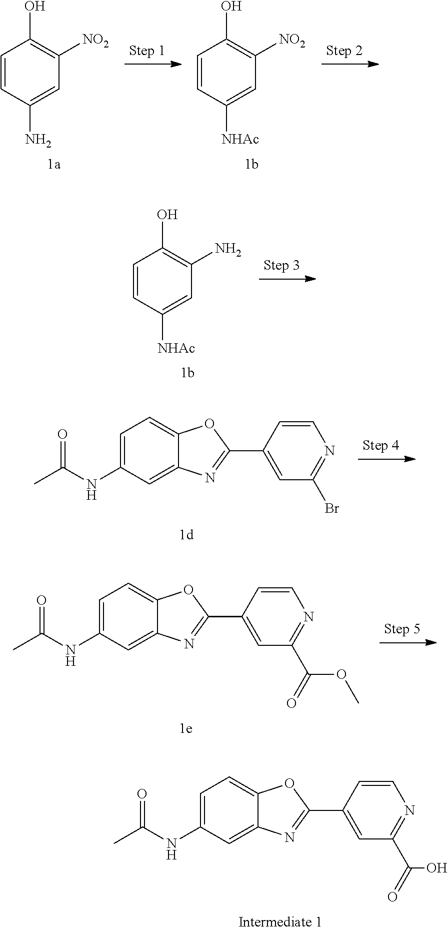

Step 1:

### N-(4-hydroxy-3-nitrophenyl)acetamide (1b)

Under nitrogen protection, anhydrous acetic acid (30 mL) and acetic anhydride (3.97 g, 39 mmol) were successively added to compound 1a (5.0 g, 32.5 mmol), and the reaction solution was warmed to 50° C.-60° C. and stirred for 3 hours. The reaction solution was poured into ice water (200 mL), stirred for 10 minutes and filtered. The filter cake was washed with water (5 mL) and dried to obtain compound 1b (5.4 g, 85%).

LC-MS (ESI): m/z=197.2 [M+H]⁺.

Step 2:

### N-(3-amino-4-hydroxyphenyl)acetamide (1c)

At room temperature, compound 1b (5.4 g, 27.62 mmol) was dissolved in anhydrous methanol (54 mL); Pd/C (1.08 g, with Pd content of 10% and water content of 50%) was added; hydrogen was introduced; and the mixture was warmed to 45° C. and reacted for 5 h. After filtration, the filtrate was concentrated to obtain compound 1c (4.11 g, 90%).

¹H NMR (400 MHz, DMSO-d₆) δ 9.40 (s, 1H), 8.70 (s, 1H), 6.92 (d, 1H), 6.58-6.51 (m, 2H), 4.70 (s, 2H), 1.94 (s, 3H).

LC-MS (ESI): m/z=167.2 [M+H]⁺.

Step 3:

### N-(2-(2-bromopyridin-4-yl)benzo[d]oxazol-5-yl)acetamide (1d)

Compound 1c (5.0 g, 30.1 mmol) was dissolved in methanol (50 mL); 2-bromopyridine-4-carbaldehyde (5.6 g, 30.1 mmol) was added; and the mixture was warmed to 70° C. and stirred for 15 h. The reaction solution was cooled to room temperature and concentrated under reduced pressure to remove methanol, and then dichloromethane (200 mL) and DDQ (8.19 g, 36.1 mmol) were successively added to the residue. The mixture was stirred for 2 h, and saturated aqueous sodium carbonate solution (100 mL) was added. The resulting solution was stirred for 10 min and filtered. The filtrate was extracted with dichloromethane (200 mL×2). The combined organic phase was washed with water (100 mL), dried over anhydrous sodium sulfate and filtered. The filtrate was concentrated under reduced pressure, and then the residue was separated and purified by column chromatography (eluent: MeOH/DCM=0%-15%) to obtain 1d (3.3 g, 33%).

¹H NMR (400 MHz, DMSO-d₆) δ 10.17 (s, 1H), 8.65 (d, 1H), 8.11 (d, 1H), 8.05 (d, 1H), 7.78 (d, 1H), 7.60 (d, 1H), 7.58 (d, 1H), 2.09 (s, 3H).

LC-MS (ESI): m/z=333.2 [M+H]⁺.

Step 4:

### Methyl 4-(5-acetamidobenzo[d]oxazol-2-yl)picolinate (1e)

Methanol (15 mL), dichloromethane (15 mL), Pd(dppf)Cl₂ (660 mg, 0.9 mmol) and triethylamine (3.64 g, 36.3 mmol) were successively added to compound 1d (3.0 g, 9.06 mmol); carbon monoxide was introduced; and then the reaction solution was warmed to 120° C. and stirred for 14 h. The reaction solution was cooled to room temperature and then filtered. The filtrate was concentrated under reduced pressure, and then the residue was separated and purified by column chromatography (eluent: MeOH/DCM=10%) to obtain 1e (1.4 g, 50%).

¹H NMR (400 MHz, DMSO-d₆) δ 10.17 (s, 1H), 8.65 (d, 1H), 8.11 (d, 1H), 8.05 (d, 1H), 7.78 (d, 1H), 7.60 (d, 1H), 7.58 (d, 1H), 3.96 (s, 3H), 2.09 (s, 3H).

LC-MS (ESI): m/z=312.1 [M+H]⁺.

Step 5:

### 4-(5-acetamidobenzo[d]oxazol-2-yl)picolinic Acid (Intermediate 1)

Anhydrous methanol (15 mL) and NaOH (0.36 g, 9.0 mmol, 5 mL) aqueous solution were successively added to compound 1e (1.4 g, 4.5 mmol). The mixture was stirred at room temperature for 5 h, concentrated under reduced pressure to remove most of methanol, adjusted to pH=2-3 by dropwise adding 1 N hydrochloric acid and filtered. The filter cake was washed with water (3 mL) and dried to obtain intermediate 1.

¹H NMR (400 MHz, DMSO-d₆) δ 10.17 (s, 1H), 8.65 (d, 1H), 8.11 (d, 1H), 8.05 (d, 1H), 7.78 (d, 1H), 7.60 (d, 1H), 7.58 (d, 1H), 2.09 (s, 3H).

LC-MS (ESI): m/z=296.1 [M−H]⁻.

Intermediate 2:

### 1-((2-methyl-2H-tetrazol-5-yl)(phenyl)methyl)piperazine

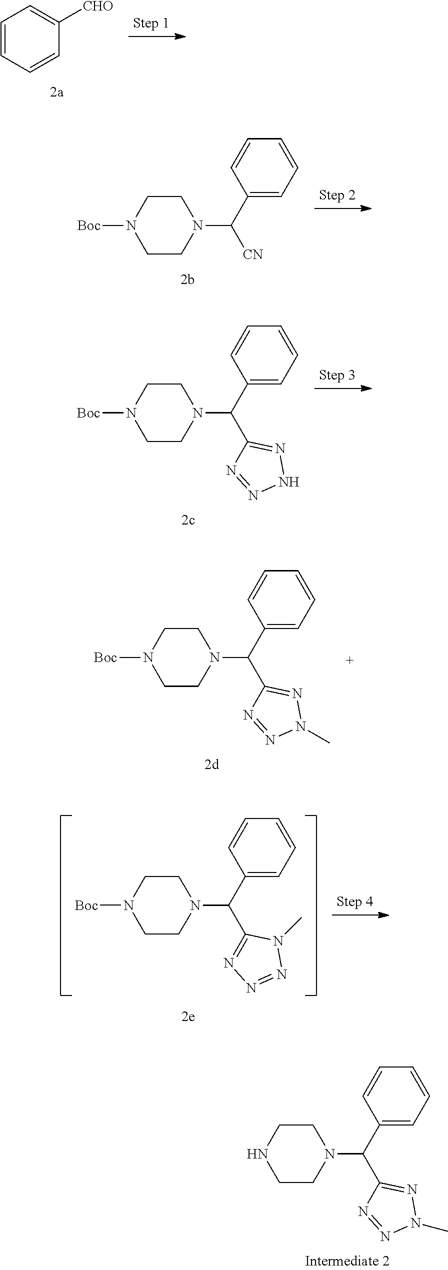

Step 1:

### tert-butyl-4-(cyano(phenyl)methyl)piperazine-1-carboxylate (2b)

Under nitrogen protection, acetonitrile (50 mL), 1-Boc-piperazine (5.0 g, 26.88 mmol), trimethylsilyl cyanide (3.2 g, 32.26 mmol) and iodine (0.68 g, 2.7 mmol) were successively added to compound 2a (2.9 g, 26.88 mmol). The reaction solution was stirred at room temperature for 15 hours, and saturated aqueous sodium carbonate solution (50 mL) was added. The residue was extracted with ethyl acetate (100 mL×2). The combined organic phase was dried over anhydrous sodium sulfate and filtered. The filtrate was concentrated under reduced pressure, and then the residue was separated and purified by column chromatography (eluent: EA/PE=10%-30%) to obtain 2b (4.0 g, 50%).

¹H NMR (400 MHz, CDCl₃) δ 7.54-7.52 (m, 2H), 7.41-7.39 (m, 3H), 4.87 (s, 1H), 3.48-3.43 (m, 4H), 2.54-2.52 (m, 4H), 1.46 (s, 9H).

LC-MS (ESI): m/z=246.2 [M+H]⁺.

Step 2:

### tert-butyl 4-(phenyl(2H-tetrazol-5-yl)methyl)piperazine-1-carboxylate (2c)

Under nitrogen protection, DMF (8 mL), toluene (24 mL), and azidotributyltin (6.6 g, 20.16 mmol) were successively added to compound 2b (4.0 g, 13.44 mmol), and the reaction solution was warmed to 130° C. and stirred for 30 h. The reaction solution was cooled to room temperature and then washed with saturated aqueous potassium carbonate solution (30 mL×2). The combined aqueous phase was adjusted to pH=2-3 with 6 N hydrochloric acid and then extracted with ethyl acetate (100 mL×2). The combined ethyl acetate phase was dried over anhydrous sodium sulfate and filtered. The filtrate was concentrated under reduced pressure to obtain 2c (1.7 g, 37.2%).

LC-MS (ESI): m/z=345.2 [M+H]⁺.

Step 3:

### tert-butyl-4-((2-methyl-2H-tetrazol-5-yl)(phenyl)methyl)piperazine-1-carboxylate (2d)

Acetonitrile (10 mL), potassium carbonate (2.0 g, 16.0 mmol), and iodomethane (1.7 g, 12.0 mmol) were successively added to compound 2c (1.0 g, 4.0 mmol). The reaction solution was stirred for 3 hours at room temperature and filtered. Water (100 mL) was added to the filtrate, and the resulting solution was extracted with ethyl acetate (100 mL×2). The combined organic phase was dried over anhydrous sodium sulfate and filtered. The filtrate was concentrated under reduced pressure, and then the residue was separated and purified by column chromatography (eluent: EA/PE=10%-20%) to obtain 2d (170 mg, 17%).

¹H NMR (400 MHz, CDCl₃) δ 7.52-7.51 (m, 2H), 7.34-7.28 (m, 3H), 4.92 (s, 1H), 4.33 (s, 3H), 2.47-2.32 (m, 4H), 3.45 (m, 4H), 1.43 (s, 9H).

LC-MS (ESI): m/z=359.2[M+H]⁺.

Step 4:

### 1-((2-methyl-2H-tetrazol-5-yl)(phenyl)methyl)piperazine (Intermediate 2)

Methanol (5 mL) and concentrated hydrochloric acid (2 mL) were successively added to compound 2d (170 mg, 0.47 mmol). The reaction solution was stirred at room temperature for 2 h and adjusted to pH=8-9 by dropwise adding saturated aqueous sodium carbonate solution. The aqueous phase was extracted with dichloromethane (50 mL×2). The combined organic phase was dried over anhydrous sodium sulfate, filtered and concentrated under reduced pressure to obtain intermediate 2 (110 mg, 90%).

¹H NMR (400 MHz, CDCl₃) δ 7.53-7.51 (m, 2H), 7.35-7.26 (m, 3H), 4.89 (s, 1H), 4.33 (s, 3H), 2.94-2.91 (m, 4H), 2.53-2.35 (m, 4H).

LC-MS (ESI): m/z=259.2[M+H]⁺.

Intermediate 3:

### Methyl 4-(5-((tert-butoxycarbonyl)amino)benzo[d]oxazol-2-yl)picolinate

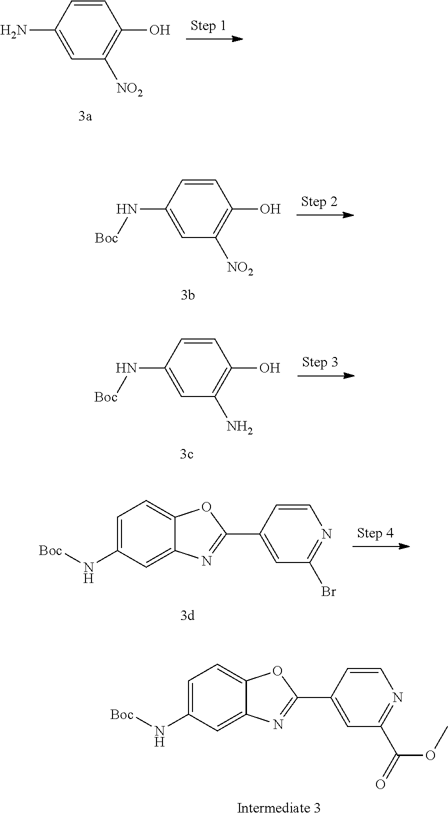 [SMILES: Nc1ccc(O)c([N+](=O)[O-])c1 ; CC(C)(C)OC(=O)Nc1ccc(O)c([N+](=O)[O-])c1 ; CC(C)(C)OC(=O)Nc1ccc(O)c(N)c1 ; CC(C)(C)OC(=O)Nc1ccc2oc(-c3ccnc(Br)c3)nc2c1 ; COC(=O)c1cc(-c2nc3cc(NC(=O)OC(C)(C)C)ccc3o2)ccn1]

Step 1:

### tert-butyl-(4-hydroxy-3-nitrophenyl)carbamate (3b)

Tetrahydrofuran (50 mL), di-tert-butyl dicarbonate (10.6 g, 48.7 mmol) were successively added to known compound 3a (5.0 g, 32.5 mmol). After the addition, the mixture was warmed to 70° C., reacted for 16 h and concentrated under reduced pressure to remove tetrahydrofuran. The resulting mixture was slurried with petroleum ether (100 mL) for 1 hour and then filtered. The filter cake was collected and dried to obtain compound 3b (6.1 g, 74%).

¹H NMR (400 MHz, CD₃OD) δ 8.25 (d, 1H), 7.56 (d, 1H), 7.06 (d, 1H), 1.52 (s, 9H).

LC-MS (ESI): m/z=255.1[M+H]⁺.

Step 2:

### tert-butyl-(3-amino-4-hydroxyphenyl)carbamate (3c)

At room temperature, compound 3b (6.1 g, 24.0 mmol) was dissolved in anhydrous methanol (60 mL); Pd/C (2.1 g, with Pd content of 10% and water content of 50%) was added; hydrogen was introduced; and the mixture was warmed to 45° C. and reacted for 5 h. After filtration, the filtrate was concentrated to obtain compound 3c (4.3 g, 80%).

LC-MS (ESI): m/z=225.1[M+H]⁺.

Step 3:

### tert-butyl (2-(2-bromopyridin-4-yl)-2,3-dihydrobenzo[d]oxazol-5-yl)carbamate (3d)

Compound 3c (4.3 g, 19.2 mmol) was dissolved in methanol (50 mL); 2-bromopyridine-4-carbaldehyde (3.6 g, 19.2 mmol) was added; and the mixture was warmed to 70° C. and stirred for 15 h. The reaction solution was cooled to room temperature and concentrated under reduced pressure to remove methanol, and then dichloromethane (200 mL) and DDQ (5.3 g, 23.0 mmol) were successively added to the residue. After the addition, the mixture was stirred for 2 h at room temperature, and saturated aqueous sodium carbonate solution (100 mL) was added. The resulting solution was stirred for 10 min and filtered. The filtrate was extracted with dichloromethane (200 mL×2). The combined organic phase was washed with water (100 mL), dried over anhydrous sodium sulfate and filtered. The filtrate was concentrated under reduced pressure, and then the residue was separated and purified by column chromatography (eluent: EA (ethyl acetate)/PE (petroleum ether)=10%-50%) to obtain 3d (4.1 g, 54%).

LC-MS (ESI): m/z=392.1[M+H]⁺.

Step 4:

### Methyl 4-(5-((tert-butoxycarbonyl)amino)benzo[d]oxazol-2-yl)picolinate (Intermediate 3)

Methanol (25 mL), dichloromethane (25 mL), Pd(dppf)Cl₂ (804.0 mg, 1.1 mmol) and triethylamine (4.24 g, 42.0 mmol) were successively added to compound 3d (4.1 g, 10.5 mmol); carbon monoxide was introduced; and then the reaction solution was warmed to 120° C. and stirred for 14 h. The reaction solution was cooled to room temperature and then filtered. The filtrate was concentrated under reduced pressure, and then the residue was separated and purified by column chromatography (eluent: EA/PE=10%-50%) to obtain intermediate 3 (3.5 g, 90%).

¹H NMR (400 MHz, CDCl₃) δ 8.95 (d, 1H), 8.89 (d, 1H), 8.26 (d, 1H), 7.86 (s, 1H), 7.54-7.47 (m, 2H), 6.67 (s, 1H), 4.08 (s, 3H), 1.55 (s, 9H).

LC-MS (ESI): m/z=370.1[M+H]⁺.

Intermediate 4:

### 4-(5-(cyclopropanecarboxamido)benzo[d]oxazol-2-yl)picolinic Acid

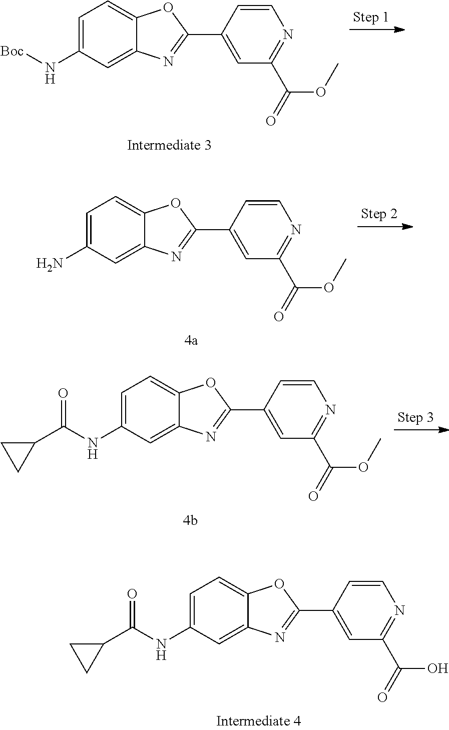 [SMILES: COC(=O)c1cc(-c2nc3cc(NC(=O)OC(C)(C)C)ccc3o2)ccn1 ; COC(=O)c1cc(-c2nc3cc(N)ccc3o2)ccn1 ; COC(=O)c1cc(-c2nc3cc(NC(=O)C4CC4)ccc3o2)ccn1 ; O=C(O)c1cc(-c2nc3cc(NC(=O)C4CC4)ccc3o2)ccn1]

Step 1:

### Methyl-4-(5-aminobenzo[d]oxazol-2-yl)picolinate (4a)

Intermediate 3 (600.0 mg, 1.62 mmol) was dissolved in dichloromethane (5 mL), and trifluoroacetic acid (2 mL) was added. After the addition, the mixture was stirred for 2 hours at room temperature, adjusted to pH=8-9 with saturated aqueous sodium carbonate solution and extracted twice with dichloromethane (50 mL×2). The organic phase was combined, dried and filtered. The filtrate was concentrated to obtain compound 4a (396.0 mg, 90%).

LC-MS (ESI): m/z=270.1[M+H]⁺.

Step 2:

### Methyl-4-(5-(cyclopropanecarboxamido)benzo[d]oxazol-2-yl)picolinate (4b)

Under nitrogen protection, compound 4a (196.0 mg, 0.72 mmol) was dissolved in dichloromethane (5 mL), and triethylamine (109.8 mg, 1.08 mmol) was added. The mixture was cooled to 0-5° C. in an ice bath, and cyclopropanecarboxylic acid chloride (82.5 mg, 0.80 mmol) was added. After the addition, the mixture was reacted for 1 hour; water (10 mL) was added; and the resulting solution was extracted twice with dichloromethane (50 mL×2). The organic phase was combined, dried and filtered. The filtrate was concentrated to obtain compound 4b (220 mg, 90%).

LC-MS (ESI): m/z=338.1[M+H]⁺.

Step 3:

### 4-(5-(cyclopropanecarboxamido)benzo[d]oxazol-2-yl)picolinic Acid (Intermediate 4)

Compound 4b (220 mg, 0.65 mmol) was dissolved in methanol (2 mL), and NaOH (52.0 mg, 1.3 mmol, 0.5 mL) aqueous solution was added. The mixture was stirred at room temperature for 5 hours, concentrated under reduced pressure to remove most of methanol, adjusted to pH=2-3 by dropwise adding 1 N hydrochloric acid and filtered. The filter cake was washed with water (3 mL) and dried to obtain intermediate 4 (178.5 mg, 85%).

LC-MS (ESI): m/z=322.1[M−H]⁻.

Intermediate 5:

### 4-(5-(2-oxopyrrolidin-1-yl)benzo[d]oxazol-2-yl)picolinic Acid

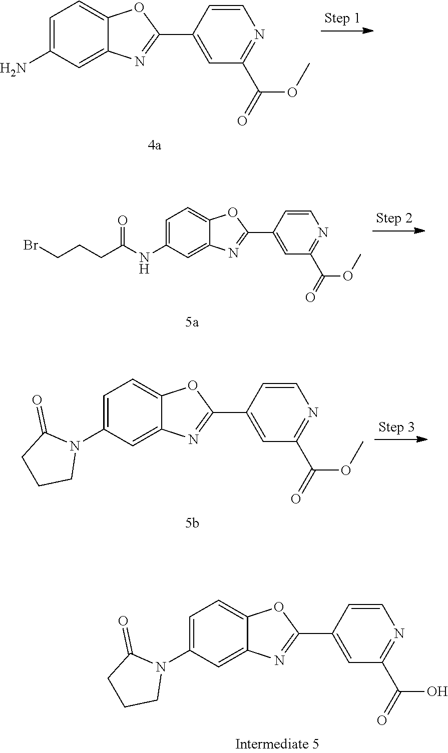 [SMILES: COC(=O)c1cc(-c2nc3cc(N)ccc3o2)ccn1 ; COC(=O)c1cc(-c2nc3cc(NC(=O)CCCBr)ccc3o2)ccn1 ; COC(=O)c1cc(-c2nc3cc(N4CCCC4=O)ccc3o2)ccn1 ; O=C(O)c1cc(-c2nc3cc(N4CCCC4=O)ccc3o2)ccn1]

Step 1:

### Methyl 4-(5-(4-bromobutanamido)benzo[d]oxazol-2-yl)picolinate (5a)

Under nitrogen protection, compound 4a (200 mg, 0.74 mmol) was dissolved in dichloromethane (5 mL), and triethylamine (150.0 mg, 1.48 mmol) was added. The mixture was cooled to 0-5° C., and 4-bromobutanoyl chloride (164.5 mg, 0.89 mmol) was added. After the addition, the mixture was reacted for 1 hour; water (10 mL) was added; and the resulting solution was extracted twice with dichloromethane (50 mL×2). The organic phase was combined, dried and filtered. The filtrate was concentrated to obtain compound 5a (224 mg, 90%).

Step 2:

### Methyl-4-(5-(2-oxopyrrolidin-1-yl)benzo[d]oxazol-2-yl)picolinate (5b)

Under nitrogen protection, compound 5a (280 mg, 0.67 mmol) was dissolved in tetrahydrofuran (2 mL). The mixture was cooled to 0-5° C., and sodium hydride (80 mg, 2.0 mmol, a content of 60%) was added. After the addition, the mixture was allowed to naturally warm to room temperature and reacted for 3 hours. The reaction solution was added to ice water (10 mL), and the resulting solution was extracted twice with dichloromethane (50 mL×2). The organic phase was combined, dried and filtered. The filtrate was concentrated to obtain compound 5b (192.0 mg, 85%).

LC-MS (ESI): m/z=338.1[M+H]⁺.

Step 3:

### Methyl-4-(5-(2-oxopyrrolidin-1-yl)benzo[d]oxazol-2-yl)picolinate (Intermediate 5)

Compound 5b (192.0 mg, 0.57 mmol) was dissolved in methanol (2 mL) and NaOH (48.0 mg, 1.2 mmol, 0.5 mL) aqueous solution was added. The mixture was stirred at room temperature for 5 hours, concentrated under reduced pressure to remove most of methanol, adjusted to pH=2-3 by dropwise adding 1 N hydrochloric acid and filtered. The filter cake was washed with water (3 mL) and dried to obtain intermediate 5 (166.0 mg, 90%).

LC-MS (ESI): m/z=322.1[M−H]⁻.

Intermediate 6:

### 1-((2-(difluoromethyl)-2H-tetrazol-5-yl)(phenyl)methyl)piperazine Hydrochloride

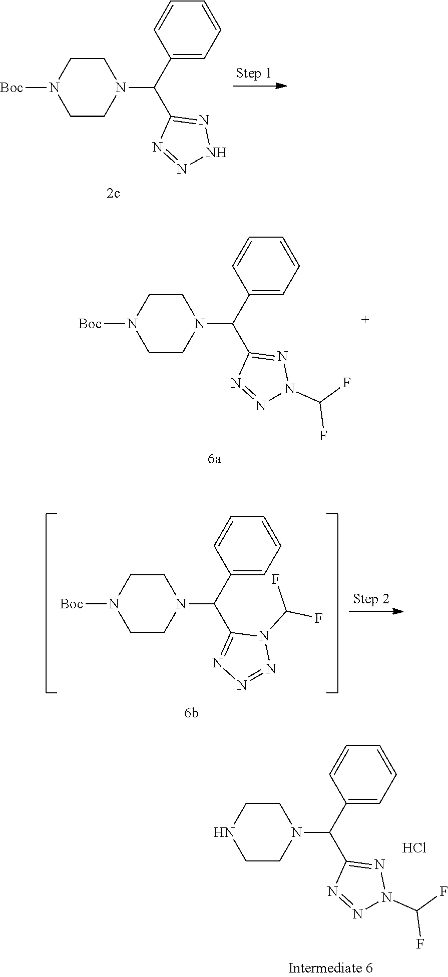

Step 1:

### tert-butyl 4-((2-(difluoromethyl)-2H-tetrazol-5-yl)(phenyl)methyl)piperazine-1-carboxylate (6a)

Acetonitrile (3 mL) was added to compound 2c (300 mg, 0.867 mmol), and the mixture was cooled to 0° C.; and then potassium hydroxide (973 mg, 17.3 mmol) and water (3 mL) were added. The reaction was stirred at 0° C. for 10 minutes, and diethyl bromofluorophosphate (463 mg, 1.734 mmol) was added quickly. The reaction solution was stirred at 0° C. for 30 minutes. Water (100 mL) was added to the reaction solution, and the resulting solution was extracted with ethyl acetate (100 mL×2). The combined organic phase was dried over anhydrous sodium sulfate and concentrated under reduced pressure, and then the residue was separated and purified by silica gel column chromatography (PE:EA=3:1) to obtain 6a (200 mg, 58%) and 6b (120 mg, 35%).

LC-MS (ESI): m/z=395.2 [M+H]⁺.

Step 2:

### 1-((2-(difluoromethyl)-2H-tetrazol-5-yl)(phenyl)methyl)piperazine Hydrochloride (Intermediate 6)

Methanol (5 mL) and concentrated hydrochloric acid (2 mL) were successively added to compound 6a (200 mg, 0.51 mmol). The reaction solution was stirred at room temperature for 2 hours. The reaction solution was directly concentrated under reduced pressure to obtain compound intermediate 6 (166 mg, 98%).

¹H NMR (400 MHz, CD₃OD) δ 8.26 (t, 1H), 7.74 (d, 2H), 7.52-7.50 (m, 3H), 6.13 (s, 1H), 3.56-3.54 (m, 4H), 3.45-3.30 (m, 4H).

LC-MS (ESI): m/z=295.2 [M+H]⁺.

Intermediate 7:

### (R/S)-1-((2-(difluoromethyl)-2H-tetrazol-5-yl)(phenyl)methyl)piperazine (Intermediate 7)

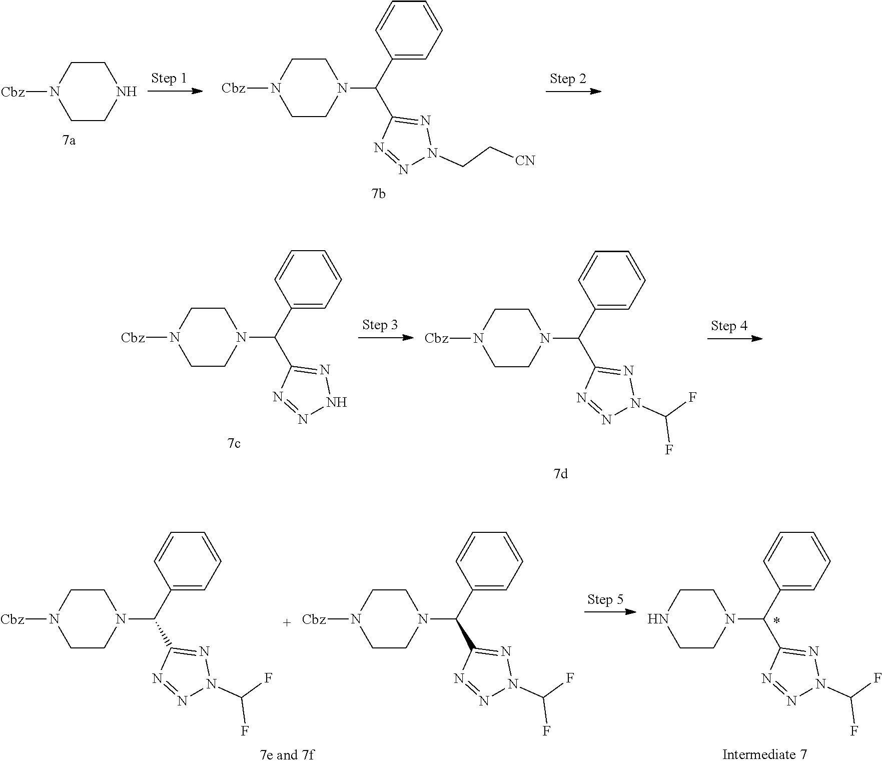

Step 1:

### benzyl4-((2-(2-cyanoethyl)-2H-tetrazol-5-yl)(phenyl)methyl)piperazine-1-carboxylate (7b)

Methanol (200 mL) and benzaldehyde (9.64 g, 91 mmol) were successively added to compound 7a (20 g, 91 mmol), and the mixture was stirred at room temperature until the system was clear. Then azidotrimethylsilane (15.7 g, 136 mmol) and 3-isocyanopropanenitrile (10.88 g, 136 mmol) were successively added, and the reaction system was stirred at room temperature for 5 hours. After completion of the reaction, the reaction solution was used directly in the next reaction.

Step 2:

### Benzyl 4-(phenyl(2H-tetrazol-5-yl)methyl)piperazine-1-carboxylate (7c)

Water (40 mL) and lithium hydroxide (4.37 g, 182 mmol) were successively added to the reaction solution of 7b, and the mixture was stirred at room temperature for 3 hours. Water (200 ml) was added to the reaction system. The impurities were extracted with ethyl acetate (100 mL×2), and then the aqueous phase was adjusted to PH=4-5 with dilute hydrochloric acid and extracted with ethyl acetate (100 mL×2). The combined organic phase was dried over anhydrous sodium sulfate, filtered and concentrated under reduced pressure to obtain 7c (30.26 g, 88%).

LC-MS (ESI): m/z=378.2 [M+H]⁺.

Step 3:

### benzyl4-((2-(difluoromethyl)-2H-tetrazol-5-yl)(phenyl)methyl)piperazine-1-carboxylate (7d)

Acetonitrile (40 mL) was added to compound 7c (30.26 g, 80 mmol). The mixture was cooled to 0° C., and then potassium hydroxide (89.8 g, 1.6 mol) and water (40 mL) were added. The reaction was stirred at 0° C. for 10 minutes, and diethyl bromofluorophosphate (42.7 g, 160 mmol) was added quickly. The reaction was stirred at 0° C. for 1 h. Water (100 mL) was added to the reaction solution, and the resulting solution was extracted with ethyl acetate (100 mL×2). The combined organic phase was dried over anhydrous sodium sulfate, filtered and concentrated under reduced pressure, and then the residue was separated and purified by silica gel column chromatography (eluent: PE/EA=1/2) to obtain 7d (19.9 g, 58%).

LC-MS (ESI): m/z=428.2 [M+H]⁺.

Step 4:

### (R)-benzyl4-((2-(difluoromethyl)-2H-tetrazol-5-yl)(phenyl)methyl)piperazine-1-carboxylate

### (S)-benzyl4-((2-(difluoromethyl)-2H-tetrazol-5-yl)(phenyl)methyl)piperazine-1-carboxylate

Compound 7d was resolved by chiral HPLC to obtain compound 7e and compound 7f, with purification conditions as follows:

(instrument name: Thar 200 preparative SFC (SFC-7); chromatographic column: ChiralPak AD, 300×50 mm I.D., 10 μm; mobile phase: A for CO₂ and B for Ethanol; gradient: B 20%; flow rate: 200 mL/min; column pressure: 100 bar; column temperature: 38° C.; absorption wavelength: 220 nm; cycle time: about 10 min); retention time for compound 7e: 3.429 min; retention time for compound 7f: 3.138 min.

Compound 7e: LC-MS (ESI): m/z=429.2 [M+H]⁺.

Compound 7f: LC-MS (ESI): m/z=429.2 [M+H]⁺.

Step 5:

### (R)-1-((2-(difluoromethyl)-2H-tetrazol-5-yl)(phenyl)methyl)piperazine (Intermediate 7)

10% palladium on carbon (1 g) and methanol (60 ml) were added to 7e (8 g, 18.7 mmol). After hydrogen replacement, the mixture was reacted at room temperature for 4 h. After completion of the reaction, the reaction mixture was filtered through celite. The filtrate was concentrated under reduced pressure to obtain intermediate 7 (5.2 g, 95%, a compound in a single configuration).

1H NMR (400 MHz, CDCl₃) δ 7.75-7.45 (m, 3H), 7.37-7.28 (m, 3H), 5.01 (s, 1H), 3.47 (s, 1H), 2.90 (t, J=4.9 Hz, 4H), 2.55-2.46 (m, 2H), 2.37-2.29 (m, 2H).

LC-MS (ESI): m/z=295.1 [M+H]⁺.

### Example 1

### N-(2-(2-(8-((2-methyl-2H-tetrazol-5-yl)(phenyl)methyl)-3,8-diazabicyclo[3.2.1]octane-3-carbonyl)pyridin-4-yl)benzo[d]oxazol-5-yl)acetamide

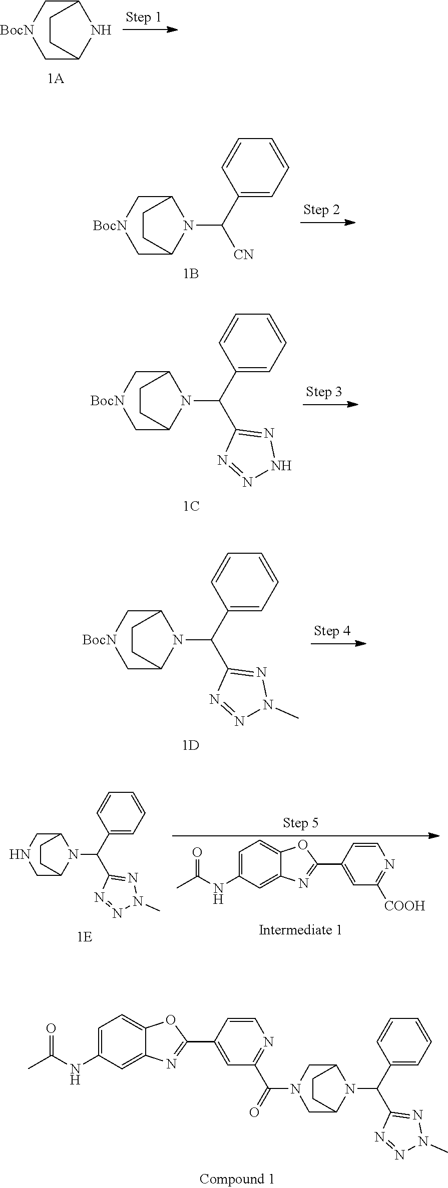

Step 1:

### tert-butyl 8-(cyano(phenyl)methyl)-3,8-diazabicyclo[3.2.1]octane-3-carboxylate (1)

Under nitrogen protection, acetonitrile (50 mL), benzaldehyde (2.5 g, 24 mmol), trimethylsilyl cyanide (3.6 g, 36 mol) and iodine (0.6 g, 2.4 mmol) were successively added to compound 1A (5.0 g, 24 mmol), and the reaction was stirred at room temperature for 16 hours. Saturated sodium bicarbonate solution (50 mL) was added, and the mixture was extracted with ethyl acetate (50 mL×2). The organic phase was combined, dried, filtered and concentrated under reduced pressure, and the residue was separated and purified by column chromatography (EA/PE: 20%-50%) to obtain compound 1B (5 g, 64%).

LC-MS (ESI): m/z=328.2 [M+H]⁺.

Step 2:

### tert-butyl 8-(phenyl(2H-tetrazol-5-yl)methyl)-3,8-diazabicyclo[3.2.1]octane-3-carboxylate (1C)

At room temperature, isopropanol (40 mL), sodium azide (0.8 g, 12.2 mmol) and zinc bromide (0.7 g, 3.0 mmol) were successively added to compound 1B (2.0 g, 6.1 mmol), and the mixture was heated to 85° C., refluxed and reacted for 16 hours. The reaction solution was cooled to room temperature, poured into water (150 mL), adjusted to pH=5-6 with hydrochloric acid (6 N) and extracted with ethyl acetate (60 mL×2). The organic phase was combined, dried, filtered and concentrated under reduced pressure, and the residue was separated and purified by column chromatography (MeOH/DCM: 2%-4%) to obtain compound 1C (0.3 g, 13%).

LC-MS (ESI): m/z=371.2 [M+H]⁺.

Step 3:

### tert-butyl 8-((2-methyl-2H-tetrazol-5-yl)(phenyl)methyl)-3,8-diazabicyclo[3.2.1]octane-3-carboxylate (1D)

At room temperature, methanol (1 mL) and tetrahydrofuran (4 mL) were added to compound 1C (0.3 g, 0.81 mmol). Trimethylsilyldiazomethane (0.5 g, 4.5 mmol) was added dropwise to the mixture at room temperature, and the reaction was stirred for 1 hour. The reaction solution was poured into water (30 mL) and extracted with ethyl acetate (20 mL×1). The organic phase was combined, dried, filtered and concentrated under reduced pressure, and the residue was separated and purified by column chromatography (EA/PE: 20%-50%) to obtain compound 1D (0.15 g, 48%).

¹H NMR (400 MHz, CDCl₃) δ 7.65-7.31 (m, 5H), 4.91 (s, 1H), 4.30 (s, 3H), 3.70-3.55 (m, 2H), 3.16-3.00 (m, 4H), 2.09-2.01 (m, 1H), 1.99-1.95 (m, 1H), 1.74-1.65 (m, 2H), 1.43 (s, 9H).

LC-MS (ESI): m/z=385.2 [M+H]⁺.

Step 4:

### 8-((2-methyl-2H-tetrazol-5-yl)(phenyl)methyl)-3,8-diazabicyclo[3.2.1]octane (1E)

At room temperature, MeOH (2 mL) and concentrated hydrochloric acid (1 mL) were added to compound 1D (0.15 g, 0.4 mmol), and the reaction was stirred for 1 hour. The reaction solution was adjusted to pH=8 with saturated sodium bicarbonate solution and extracted with ethyl acetate (10 mL×2). The organic phase was combined, dried, filtered and concentrated under reduced pressure to obtain compound 1E (85 mg, 75%).

LC-MS (ESI): m/z=285.2 [M+H]⁺.

Step 5:

### N-(2-(2-(8-((2-methyl-2H-tetrazol-5-yl)(phenyl)methyl)-3,8-diazabicyclo[3.2.1]octane-3-carbonyl)pyridin-4-yl)benzo[d]oxazol-5-yl)acetamide (Compound 1)

At room temperature, DMF (5 mL), intermediate 1 (89 mg, 0.3 mmol), HATU (0.13 mg, 0.33 mmol) and DIPEA (0.12 g, 0.9 mmol) were successively added to compound 1E (85 mg, 0.3 mmol). The reaction was stirred for 1 hour. The reaction solution was poured into water (50 mL) and extracted with ethyl acetate (20 mL×2). The organic phase was combined, dried, filtered and concentrated under reduced pressure, and the residue was separated and purified by silica gel column chromatography (MeOH/DCM: 2%-4%) to obtain compound 1 (40 mg, 24%).

¹H NMR (400 MHz, CD₃OD) δ 8.83-8.79 (m, 1H), 8.42-8.41 (m, 1H), 8.24-8.20 (m, 2H), 7.78-7.73 (m, 2H), 7.69-7.66 (m, 1H), 7.56-7.46 (m, 4H), 5.73-5.70 (m, 1H), 4.59-4.51 (t, 1H), 4.42-4.38 (d, 3H), 4.05-3.95 (m, 2H), 3.82-3.66 (m, 2H), 3.53-3.48 (m, 1H), 2.44-2.35 (m, 2H), 2.27-2.21 (m, 1H), 2.17 (s, 3H), 2.09-2.03 (m, 1H).

LC-MS (ESI): m/z=564.2 [M+H]⁺.

### Example 2

### 1-(2-(2-(4-((2-methyl-2H-tetrazol-5-yl)(phenyl)methyl)piperazine-1-carbonyl)pyridin-4-yl)benzo[d]oxazol-5-yl)pyrrolidin-2-one

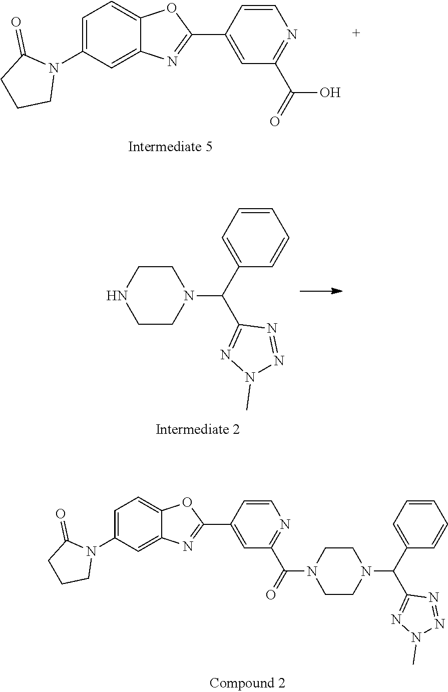 [SMILES: O=C(O)c1cc(-c2nc3cc(N4CCCC4=O)ccc3o2)ccn1 ; "+" ; Cn1nnc(C(c2ccccc2)N2CCNCC2)n1 ; Cn1nnc(C(c2ccccc2)N2CCN(C(=O)c3cc(-c4nc5cc(N6CCCC6=O)ccc5o4)ccn3)CC2)n1]

Intermediate 5 (38.7 mg, 0.12 mmol) was dissolved in N,N-dimethylformamide (1 mL), and the mixture was cooled to 0° C.; HATU (53.2 mg, 0.14 mmol) and DIPEA (46.5 mg, 0.36 mmol) were added; and then intermediate 2 (31.0 mg, 0.12 mmol) was added. After the addition, the mixture was stirred for 1 hour while the temperature was controlled at 0° C., and then ice water (5 mL) was added. The resulting solution was extracted twice with dichloromethane (20 mL×2). The organic phase was combined, dried over anhydrous sodium sulfate and filtered. The filtrate was concentrated under reduced pressure, and then the residue was separated and purified by column chromatography (eluent: EA/DCM=30%-70%) to obtain compound 2 (25 mg, 37%).

¹H NMR (400 MHz, CD₃OD) δ 8.79-8.77 (m, 1H), 8.31 (s, 1H), 8.14-8.13 (m, 1H), 8.06 (d, 1H), 7.77-7.72 (m, 2H), 7.54-7.52 (m, 2H), 7.35-7.27 (m, 3H), 5.05 (s, 1H), 4.35 (s, 3H), 4.03-3.99 (m, 2H), 3.84-3.83 (m, 2H), 3.59-3.57 (m, 2H), 2.66-2.44 (m, 6H), 2.26-2.19 (m, 2H).

LC-MS (ESI): m/z=564.2 [M+H]⁺.

### Example 3

### N-(2-(2-(5-((2-methyl-2H-tetrazol-5-yl)(phenyl)methyl)octahydropyrrolo[3,4-c]pyrrole-2-carbonyl)pyridin-4-yl)benzo[d]oxazol-5-yl)acetamide

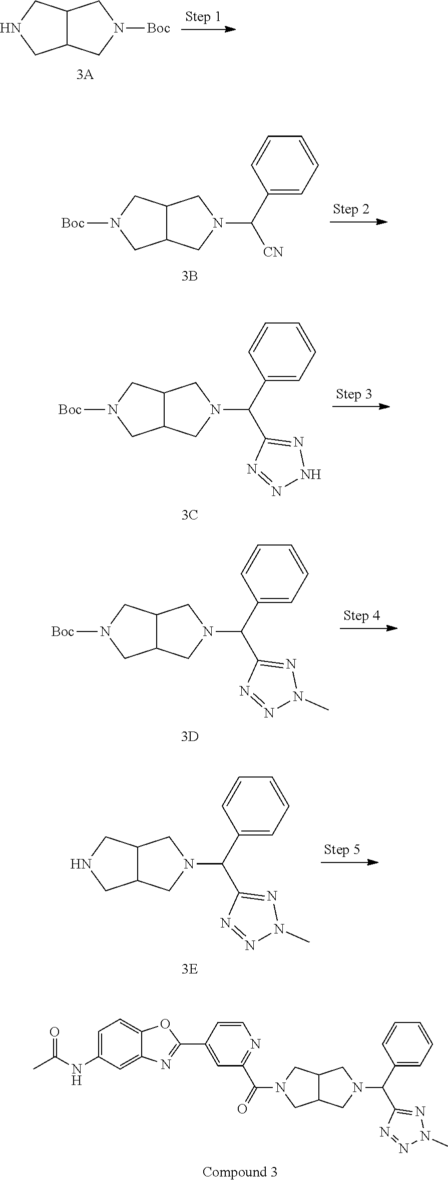 [SMILES: CC(C)(C)OC(=O)N1CC2CNCC2C1 ; CC(C)(C)OC(=O)N1CC2CN(C(C#N)c3ccccc3)CC2C1 ; CC(C)(C)OC(=O)N1CC2CN(C(c3ccccc3)c3nn[nH]n3)CC2C1 ; Cn1nnc(C(c2ccccc2)N2CC3CN(C(=O)OC(C)(C)C)CC3C2)n1 ; Cn1nnc(C(c2ccccc2)N2CC3CNCC3C2)n1 ; CC(=O)Nc1ccc2oc(-c3ccnc(C(=O)N4CC5CN(C(c6ccccc6)c6nnn(C)n6)CC5C4)c3)nc2c1]

Step 1:

### tert-butyl 5-(cyano(phenyl)methyl)hexahydropyrrolo[3,4-c]pyrrole-2(1H)-carboxylate (3B)

Under nitrogen protection, acetonitrile (50 mL), benzaldehyde (2.5 g, 23.6 mmol), trimethylsilyl cyanide (2.8 g, 28.3 mmol) and iodine (0.6 g, 2.4 mmol) were successively added to compound 3A (5.0 g, 23.6 mmol). The reaction was stirred at room temperature for 15 hours, and saturated aqueous sodium carbonate solution (50 mL) was added. The residue was extracted with ethyl acetate (100 mL×2). The combined organic phase was dried over anhydrous sodium sulfate and filtered. The filtrate was concentrated under reduced pressure, and then the residue was separated and purified by column chromatography (eluent: EA/PE=10%-30%) to obtain 3B (6.0 g, 77%).

¹H NMR (400 MHz, CDCl₃) δ 7.79 (d, 2H), 7.55-7.25 (m, 3H), 4.87 (s, 1H), 3.48-3.43 (m, 2H), 3.22-3.20 (m, 2H), 2.70-2.62 (m, 4H), 2.40-2.27 (m, 2H), 1.46 (s, 9H).

LC-MS (ESI): m/z=272.2 [M+H]⁺.

Step 2:

### tert-butyl5-(phenyl(2H-tetrazol-5-yl)methyl)hexahydropyrrolo[3,4-c]pyrrole-2(1H)-carboxylate (3C)

Under nitrogen protection, DMF (6 mL), toluene (18 mL), and azidotributyltin (4.6 g, 13.8 mmol) were successively added to compound 3B (3.0 g, 9.2 mmol), and the reaction solution was warmed to 130° C. and stirred for 30 hours. The reaction solution was cooled to room temperature and then washed with saturated aqueous potassium carbonate solution (30 mL×2). The combined aqueous phase was adjusted to pH=2-3 with 6 N hydrochloric acid and then extracted with ethyl acetate (100 mL×2). The combined organic phase was dried over anhydrous sodium sulfate and filtered. The filtrate was concentrated under reduced pressure to obtain 3C (1.2 g, 35%).

LC-MS (ESI): m/z=371.1 [M+H]⁺.

Step 3:

### tert-butyl5-((2-methyl-2H-tetrazol-5-yl)(phenyl)methyl)hexahydropyrrolo[3,4-c]pyrrole-2(1H)-carboxylate (3D)

Acetonitrile (15 mL), potassium carbonate (2.6 g, 12.9 mmol), and iodomethane (0.9 g, 6.5 mmol) were successively added to compound 3C (1.2 g, 3.24 mmol). The reaction solution was stirred for 3 hours at room temperature and filtered. Water (100 mL) was added to the filtrate, and then the resulting solution was extracted with ethyl acetate (100 mL×2). The combined organic phase was dried over anhydrous sodium sulfate and filtered. The filtrate was concentrated under reduced pressure, and then the residue was separated and purified by column chromatography (eluent: EA/PE=10%-30%) to obtain 3D (300 mg, 25%).

¹H NMR (400 MHz, CDCl₃) δ 7.79 (d, 2H), 7.55-7.25 (m, 3H), 4.87 (s, 1H), 4.30 (s, 3H), 3.48-3.43 (m, 2H), 3.22-3.20 (m, 2H), 2.70-2.62 (m, 4H), 2.40-2.27 (m, 2H), 1.46 (s, 9H).

LC-MS (ESI): m/z=371.2 [M+H]⁺.

Step 4:

### 2-((2-methyl-2H-tetrazol-5-yl)(phenyl)methyl)octahydropyrrolo[3,4-c]pyrrole (3E)

Methanol (5 mL) and concentrated hydrochloric acid (2 mL) were successively added to compound 3D (300 mg, 1.05 mmol). The reaction solution was stirred at room temperature for 2 hours and adjusted to pH=8-9 by dropwise adding saturated aqueous sodium carbonate solution. The residue was extracted with dichloromethane (50 mL×2). The combined organic phase was dried over anhydrous sodium sulfate and filtered. The filtrate was concentrated under reduced pressure to obtain compound 3E (207 mg, 93%).

LC-MS (ESI): m/z=285.2[M+H]⁺.

Step 5:

### N-(2-(2-(5-((2-methyl-2H-tetrazol-5-yl)(phenyl)methyl)octahydropyrrolo[3,4-c]pyrrole-2-carbonyl)pyridin-4-yl)benzo[d]oxazol-5-yl)acetamide (Compound 3)

Intermediate 1 (194.0 mg, 0.65 mmol) was dissolved in N,N-dimethylformamide (2 mL), and the mixture was cooled to 0° C.; HATU (296.4 mg, 0.78 mmol) and DIPEA (251.5 mg, 1.95 mmol) were added; and then compound 3E (207.0 mg, 0.72 mmol) was added. After the addition, the mixture was stirred for 1 hour while the temperature was controlled at 0° C., and then ice water (10 mL) was added. The resulting solution was extracted twice with dichloromethane (50 mL×2). The organic phase was combined, dried over anhydrous sodium sulfate and filtered. The filtrate was concentrated under reduced pressure, and then the residue was separated and purified by column chromatography (eluent: EA/DCM=30%-70%) to obtain compound 3 (150 mg, 39%).

¹H NMR (400 MHz, CDCl₃) δ 8.73-8.66 (m, 1H), 8.49 (d, 1H), 8.11-8.05 (m, 2H), 8.00 (s, 1H), 7.68-7.67 (m, 2H), 7.54 (d, 5H), 5.78 (d, 1H), 4.35 (d, 3H), 4.07-3.65 (m, 6H), 3.33-3.26 (m, 3H), 2.97-2.86 (m, 1H), 2.22 (s, 3H).

LC-MS (ESI): m/z=564.2[M+H]⁺.

### Example 4

### N-(2-(2-((1R,5S)-3-((2-methyl-2H-tetrazol-5-yl)(phenyl)methyl)-3,8-diazabicyclo[3.2.1]octane-8-carbonyl)pyridin-4-yl)benzo[d]oxazol-5-yl)cyclopropanecarboxamide

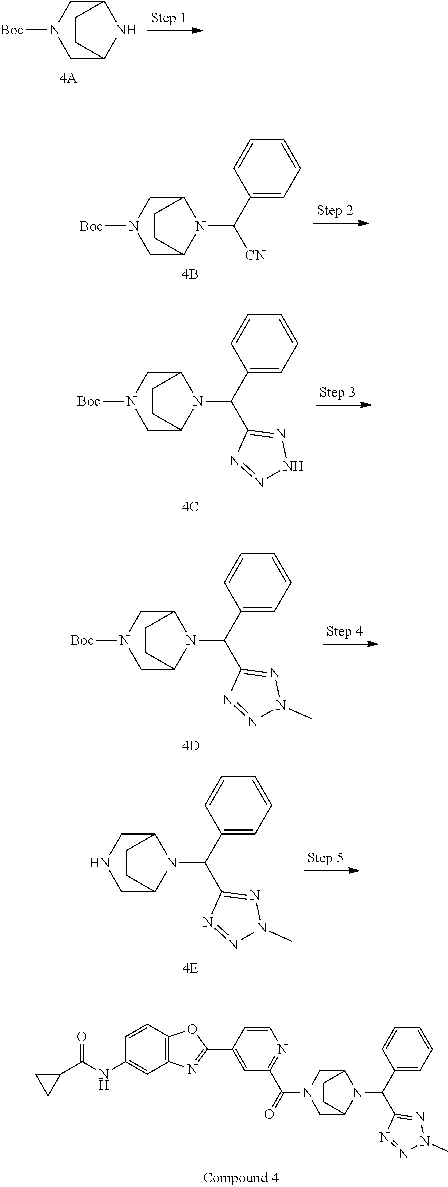 [SMILES: CC(C)(C)OC(=O)N1CC2CCC(C1)N2 ; CC(C)(C)OC(=O)N1CC2CCC(C1)N2C(C#N)c1ccccc1 ; CC(C)(C)OC(=O)N1CC2CCC(C1)N2C(c1ccccc1)c1nn[nH]n1 ; Cn1nnc(C(c2ccccc2)N2C3CCC2CN(C(=O)OC(C)(C)C)C3)n1 ; Cn1nnc(C(c2ccccc2)N2C3CCC2CNC3)n1 ; Cn1nnc(C(c2ccccc2)N2C3CCC2CN(C(=O)c2cc(-c4nc5cc(NC(=O)C6CC6)ccc5o4)ccn2)C3)n1]

Step 1:

### tert-butyl(1R,5S)-3-(cyano(phenyl)methyl)-3,8-diazabicyclo[3.2.1]octane-8-carboxylate (4B)

Compound 4B (1.4 g, 90%) was obtained by using compound 4A (1.0 g, 4.71 mmol) as a starting material with reference to the same operating method as that of compound 1.

¹H NMR (400 MHz, CDCl₃) δ 7.51 (d, 2H), 7.42-7.35 (m, 3H), 4.80 (s, 1H), 4.32-4.09 (m, 2H), 2.89-2.72 (m, 2H), 2.42-2.25 (m, 2H), 1.84-1.47 (m, 4H), 1.43 (s, 9H).

LC-MS (ESI): m/z=328.1 [M+H]⁺.

Step 2:

### tert-butyl-(1R,5S)-3-(phenyl(2H-tetrazol-5-yl)methyl)-3,8-diazabicyclo[3.2.1]octane-8-carboxylate (4C)

Compound 4C (480 mg, 31%) was obtained by using compound 4B (1.4 g, 4.24 mmol) as a starting material with reference to the same operating method as that of compound 1.

LC-MS (ESI): m/z=371.1 [M+H]⁺.

Step 3:

### tert-butyl(1R,5S)-3-((2-methyl-2H-tetrazol-5-yl)(phenyl)methyl)-3,8-diazabicyclo[3.2.1]octane-8-carboxylate (4D)

Compound 4D (110 mg, 29%) was obtained by using compound 4C (480 mg, 1.30 mmol) as a starting material with reference to the same operating method as that of compound 1.

¹H NMR (400 MHz, CDCl₃) δ 7.52 (d, 2H), 7.34-7.28 (m, 3H), 4.90 (s, 1H), 4.31 (s, 3H), 4.15-4.09 (m, 2H), 2.89-2.25 (m, 4H), 1.99-1.83 (m, 4H), 1.43 (s, 9H).

LC-MS (ESI): m/z=385.1 [M+H]⁺.

Step 4:

### (1R,5S)-3-((2-methyl-2H-tetrazol-5-yl)(phenyl)methyl)-3,8-diazabicyclo[3.2.1]octane (4E)

Compound 4E (70 mg, 86%) was obtained by using compound 4D (110 mg, 0.39 mmol) as a starting material with reference to the same operating method as that of compound 1.

LC-MS (ESI): m/z=385.1 [M+H]⁺.

Step 5:

### N-(2-(2-((1R,5S)-3-((2-methyl-2H-tetrazol-5-yl)(phenyl)methyl)-3,8-diazabicyclo[3.2.1]octane-8-carbonyl)pyridin-4-yl)benzo[d]oxazol-5-yl)cyclopropanecarboxamide (Compound 4)

Intermediate 4 (32.0 mg, 0.10 mmol) was dissolved in N,N-dimethylformamide (2 mL), and the mixture was cooled to 0° C.; HATU (35.5 mg, 0.12 mmol) and DIPEA (38.7 mg, 0.3 mmol) were added; and then compound 4E (30.0 mg, 0.11 mmol) was added. After the addition, the mixture was stirred for 1 hour while the temperature was controlled at 0° C., and then ice water (2 mL) was added. The resulting solution was extracted twice with dichloromethane (25 mL×2). The organic phase was combined, dried over anhydrous sodium sulfate and filtered. The filtrate was concentrated under reduced pressure, and then the residue was separated and purified by column chromatography (eluent: EA/DCM=30%-70%) to obtain compound 4 (25 mg, 41%).

¹H NMR (400 MHz, CD₃OD) δ 8.79 (s, 1H), 8.45 (s, 1H), 8.18 (d, 2H), 7.65-7.51 (m, 4H), 7.37-7.26 (m, 3H), 5.13 (d, 1H), 4.65 (s, 1H), 4.36 (d, 3H), 2.92-2.50 (m, 4H), 2.14-1.97 (m, 4H), 1.83-1.76 (m, 1H), 1.35-1.26 (m, 1H), 0.99-0.98 (m, 2H), 0.90-0.87 (m, 2H).

LC-MS (ESI): m/z=590.2 [M+H]⁺.

### Example 5

### N-(2-(2-(7-((2-methyl-2H-tetrazol-5-yl)(phenyl)methyl)-4,7-diazaspiro[2.5]octane-4-carbonyl)pyridin-4-yl)benzo[d]oxazol-5-yl)acetamide

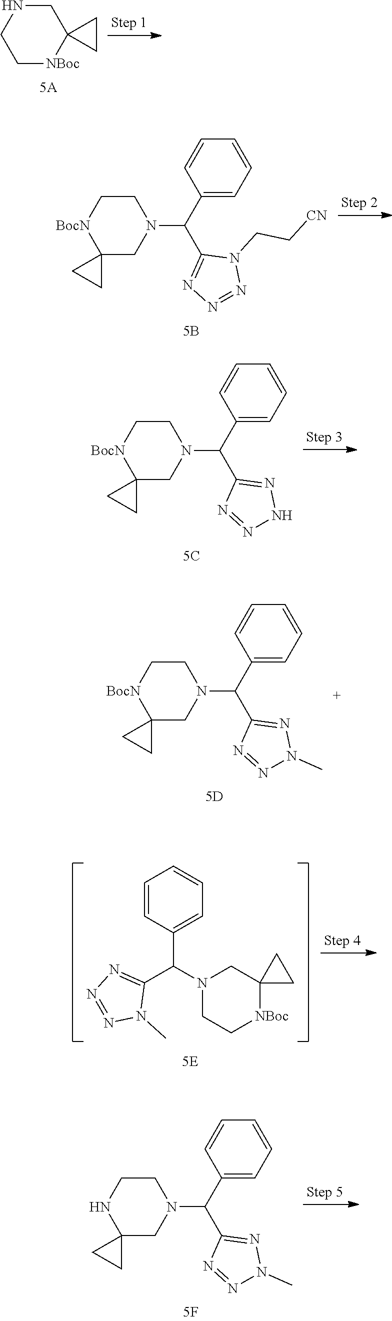

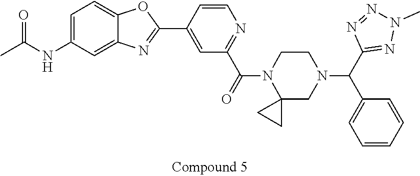 [SMILES: CC(=O)Nc1ccc2oc(-c3ccnc(C(=O)N4CCN(C(c5ccccc5)c5nnn(C)n5)CC45CC5)c3)nc2c1]

Step 1:

### tert-butyl 7-((1-(2-cyanoethyl)-1H-tetrazol-5-yl)(phenyl)methyl)-4,7-diazaspiro[2.5]octane-4-carboxylate (5B)

Compound 5A (100 mg, 0.47 mmol) was dissolved in methanol (10 mL), and benzaldehyde (50 mg, 0.47 mmol) was added at room temperature. After the mixture was reacted for ten minutes, azidotrimethylsilane (108 mg, 0.94 mmol), 3-isocyanopropanenitrile (75 mg, 0.94 mmol) were added successively. The reaction solution was stirred at room temperature for 2 hours, and water (50 mL) was added. The residue was extracted with ethyl acetate (100 mL×2). The combined organic phase was dried over anhydrous sodium sulfate and concentrated under reduced pressure, and then the residue was separated and purified by silica gel column chromatography (PE:EA=4:1) to obtain 5B (195 mg, 98%).

LC-MS (ESI): m/z=423.3 [M+H]⁺.

Step 2:

### tert-butyl 7-(phenyl(2H-tetrazol-5-yl)methyl)-4,7-diazaspiro[2.5]octane-4-carboxylate (5C)

Compound 5B (195 mg, 0.46 mmol) was dissolved in a mixed solvent of tetrahydrofuran (4 mL) and water (1 mL), and lithium hydroxide (55 mg, 2.30 mmol) was added at room temperature. The reaction solution was stirred at room temperature for 0.5 hours and concentrated under reduced pressure, and then the residue was separated and purified by silica gel column chromatography (PE:EA=1:1) to obtain 5C (169 mg, 99%).

LC-MS (ESI): m/z=371.2 [M+H]⁺.

Step 3:

### tert-butyl 7-((2-methyl-2H-tetrazol-5-yl)(phenyl)methyl)-4,7-diazaspiro[2.5]octane-4-carboxylate (5D)

Compound 5D (100 mg, 57%) was obtained by using compound 5C (169 mg, 0.46 mmol) as a starting material with reference to the same step as that of compound 1.

LC-MS (ESI): m/z=385.2 [M+H]⁺.

Step 4:

### 7-((2-methyl-2H-tetrazol-5-yl)(phenyl)methyl)-4,7-diazaspiro[2.5]octane (5F)

With reference to compound 1, compound 5F (97 mg, 100%) was obtained by the same operating step using compound 5D (100 mg, 0.34 mmol) as a starting material.

LC-MS (ESI): m/z=285.2 [M+H]⁺.

Step 5:

### N-(2-(2-(7-((2-methyl-2H-tetrazol-5-yl)(phenyl)methyl)-4,7-diazaspiro[2.5]octane-4-carbonyl)pyridin-4-yl)benzo[d]oxazol-5-yl)acetamide (Compound 5)

Compound 5 (40 mg, 14%) was obtained by using compound 5F (169 mg, 0.45 mmol) as a starting material with reference to the same step as that of compound 1.

¹H NMR (400 MHz, CDCl₃) δ 8.82-8.56 (m, 1H), 8.267 (s, 1H), 8.17-7.85 (m, 3H), 7.72 (s, 2H), 7.59-7.29 (m, 5H), 5.86 (s, 1H), 5.50-4.94 (m, 5H), 4.38 (s, 3H), 3.40 (s, 1H), 2.21 (s, 3H), 1.46-1.15 (m, 4H).

LC-MS (ESI): m/z=564.3 [M+H]⁺.

### Example 6

### N-(2-(2-((1R,4R)-5-((2-methyl-2H-tetrazol-5-yl)(phenyl)methyl)-2,5-diazabicyclo[2.2.1]heptane-2-carbonyl)pyridin-4-yl)benzo[d]oxazol-5-yl)acetamide

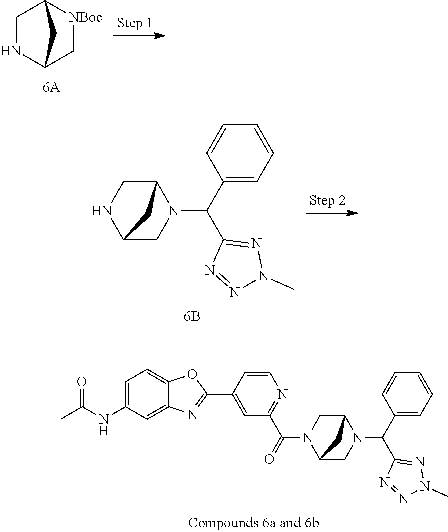 [SMILES: CC(C)(C)OC(=O)N1C[C@H]2C[C@@H]1CN2 ; Cn1nnc(C(c2ccccc2)N2C[C@H]3C[C@@H]2CN3)n1 ; CC(=O)Nc1ccc2oc(-c3ccnc(C(=O)N4C[C@H]5C[C@@H]4CN5C(c4ccccc4)c4nnn(C)n4)c3)nc2c1]

Step 1:

### (6R,4R)-2-((2-methyl-2H-tetrazol-5-yl)(phenyl)methyl)-2,5-diazabicyclo[2.2.1]heptane (6B)

Benzaldehyde 2a and 6A were used as starting materials to obtain 6B according to the synthetic step of compound 1.

LC-MS (ESI): m/z=271.2[M+H]⁺.

Step 2:

### N-(2-(2-((1R,4R)-5-((2-methyl-2H-tetrazol-5-yl)(phenyl)methyl)-2,5-diazabicyclo[2.2.1]heptane-2-carbonyl)pyridin-4-yl)benzo[d]oxazol-5-yl)acetamide (Compound 6a and Compound 6b)

Under nitrogen, dichloromethane (15 mL), DIPEA (0.13 g, 1 mmol), HATU (0.23 g, 0.6 mmol), and 6B (0.13 g, 0.5 mmol) were successively added to intermediate 1 (0.15 g, 0.5 mmol) at room temperature, and the mixture was stirred at room temperature for 1 h. After the reaction was completed, saturated aqueous sodium bicarbonate solution (30 mL) was added to the reaction solution. The mixture was extracted with dichloromethane (30 mL×1) and allowed to stand for phase separation. The aqueous phase was washed with dichloromethane (50 mL), and the combined organic phase was dried over anhydrous sodium sulfate and concentrated under reduced pressure to obtain a crude product, which was subjected to preparative high-performance liquid chromatography to obtain compounds 6a and 6b. Preparation method:

instrument: waters 2767 (preparative liquid phase chromatographic instrument); chromatographic column: Xbridge@ Prep C18 (19 mm×250 mm). The sample was dissolved in DMF and filtered with a 0.45 m filter to prepare a sample solution. Preparative chromatography conditions: a. mobile phases A and B: mobile phase A: acetonitrile; mobile phase B: containing 0.05% ammonia water/water; b. gradient elution: the content of mobile phase A rises from 10% to 70%; c. flow rate: 12 ml/min; elution time: 22 min; retention time for compound 6a: 12.36 min; retention time for compound 6b: 13.13 min.

Compound 6a:

¹H NMR (400 MHz, DMSO-d₆) δ 10.16 (s, 1H), 8.84 (s, 1H), 8.45 (s, 1H), 8.25-8.13 (m, 2H), 7.83-7.76 (m, 1H), 7.65-7.50 (m, 3H), 7.40-7.22 (m, 3H), 4.81 (s, 1H), 4.30 (s, 3H), 3.78-3.67 (m, 2H), 3.42-3.35 (m, 2H), 2.93-2.87 (m, 1H), 2.68-2.62 (m, 1H), 2.09 (s, 3H), 1.93-1.78 (m, 1H), 1.78-1.71 (m, 1H).

LC-MS (ESI): m/z=550.3 [M+H]⁺.

Compound 6b:

¹H NMR (400 MHz, DMSO-d₆) δ 10.17 (s, 1H), 8.84 (s, 1H), 8.47 (s, 1H), 8.30-8.13 (m, 2H), 7.83-7.77 (m, 1H), 7.62-7.58 (m, 3H), 7.40-7.22 (m, 3H), 4.83 (s, 1H), 4.30 (s, 3H), 3.76-3.69 (m, 2H), 3.39-3.35 (m, 2H), 2.83-2.78 (m, 1H), 2.69-2.61 (m, 1H), 2.09 (s, 3H), 1.96-1.90 (m, 1H), 1.79-1.72 (m, 1H).

LC-MS (ESI): m/z=550.3 [M+H]⁺.

### Example 7

### N-(2-(2-(4-((2-methyl-2H-tetrazol-5-yl)(phenyl)methyl)-4,7-diazaspiro[2.5]octane-7-carbonyl)pyridin-4-yl)benzo[d]oxazol-5-yl)acetamide

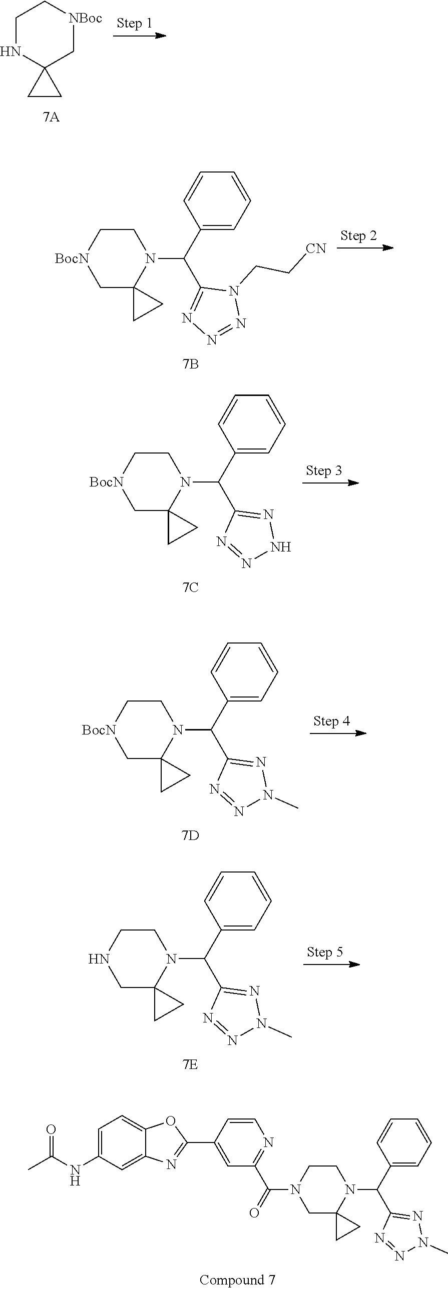 [SMILES: CC(C)(C)OC(=O)N1CCNC2(CC2)C1 ; CC(C)(C)OC(=O)N1CCN(C(c2ccccc2)c2nnnn2CCC#N)C2(CC2)C1 ; CC(C)(C)OC(=O)N1CCN(C(c2ccccc2)c2nn[nH]n2)C2(CC2)C1 ; Cn1nnc(C(c2ccccc2)N2CCN(C(=O)OC(C)(C)C)CC23CC3)n1 ; Cn1nnc(C(c2ccccc2)N2CCNCC23CC3)n1 ; CC(=O)Nc1ccc2oc(-c3ccnc(C(=O)N4CCN(C(c5ccccc5)c5nnn(C)n5)C5(CC5)C4)c3)nc2c1]

Step 1:

### tert-butyl 4-((1-(2-cyanoethyl)-1H-tetrazol-5-yl)(phenyl)methyl)-4,7-diazaspiro[2.5]octane-7-carboxylate (7B)

Compound 7A (200 mg, 0.94 mmol) was dissolved in methanol (7 mL), and benzaldehyde (100 mg, 0.94 mmol) was added at room temperature. After the mixture was reacted for ten minutes, azidotrimethylsilane (216 mg, 1.88 mmol), 3-isocyanopropanenitrile (150 mg, 1.88 mmol) were added successively. The reaction solution was stirred at room temperature for 2 hours, and water (50 mL) was added. The residue was extracted with ethyl acetate (100 mL×2). The combined organic phase was dried over anhydrous sodium sulfate and concentrated under reduced pressure, and then the residue was separated and purified by silica gel column chromatography (PE:EA=4:1) to obtain 7B (320 mg, 81%).

LC-MS (ESI): m/z=423.3 [M+H]⁺.

Step 2:

### tert-butyl 4-(phenyl(2H-tetrazol-5-yl)methyl)-4,7-diazaspiro[2.5]octane-7-carboxylate (7C)

Compound 7B (320 mg, 0.76 mmol) was dissolved in a mixed solvent of tetrahydrofuran (4 mL) and water (1 mL), and lithium hydroxide monohydrate (156 mg, 3.80 mmol) was added at room temperature. The reaction solution was stirred at room temperature for 0.5 hours and concentrated under reduced pressure, and then the residue was separated and purified by silica gel column chromatography (PE:EA=2:1) to obtain 7C (270 mg, 96%).

LC-MS (ESI): m/z=371.2 [M+H]⁺.

Step 3:

### tert-butyl 4-((2-methyl-2H-tetrazol-5-yl)(phenyl)methyl)-4,7-diazaspiro[2.5]octane-7-carboxylate (7D)

Compound 7D (130 mg, 46%) was obtained by using compound 7C (270 mg, 0.73 mmol) as a starting material with reference to the same step as that of compound 1.

LC-MS (ESI): m/z=385.2 [M+H]⁺.

Step 4:

### 4-((2-methyl-2H-tetrazol-5-yl)(phenyl)methyl)-4,7-diazaspiro[2.5]octane (7E)

Compound 7E (110 mg, 100%) was obtained by using compound 7D (130 mg, 0.34 mmol) as a starting material with reference to the same step as that of compound 1.

LC-MS (ESI): m/z=285.2 [M+H]⁺.

Step 5:

### N-(2-(2-(4-((2-methyl-2H-tetrazol-5-yl)(phenyl)methyl)-4,7-diazaspiro[2.5]octane-7-carbonyl)pyridin-4-yl)benzo[d]oxazol-5-yl)acetamide (compound 7)

Compound 7 (50 mg, 26%) was obtained by using compound 7E (110 mg, 0.34 mmol) as a starting material with reference to the same step as that of compound 1.

¹H NMR (400 MHz, CD₃OD) δ 8.76-8.80 (m, 1H), 8.31 (s, 1H), 8.17-8.20 (m, 2H), 7.55-7.68 (m, 4H), 7.24-7.36 (m, 3H), 5.91 (s, 1H), 4.35 (s, 1.5H), 4.32 (s, 1.5H), 3.82-3.96 (m, 1H), 3.63-3.72 (m, 1H), 3.38-3.46 (m, 1H), 3.04-3.21 (m, 2H), 2.93-2.96 (m, 1H), 2.17 (s, 3H), 0.53-0.59 (m, 1H), 0.37-0.46 (m, 2H), 0.18-0.30 (m, 1H).

LC-MS (ESI): m/z=564.3 [M+H]⁺.

### Example 8

### N-(2-(2-(7-((2-methyl-2H-tetrazol-5-yl)(phenyl)methyl)-2,7-diazaspiro[3.5]nonane-2-carbonyl)pyridin-4-yl)benzo[d]oxazol-5-yl)acetamide

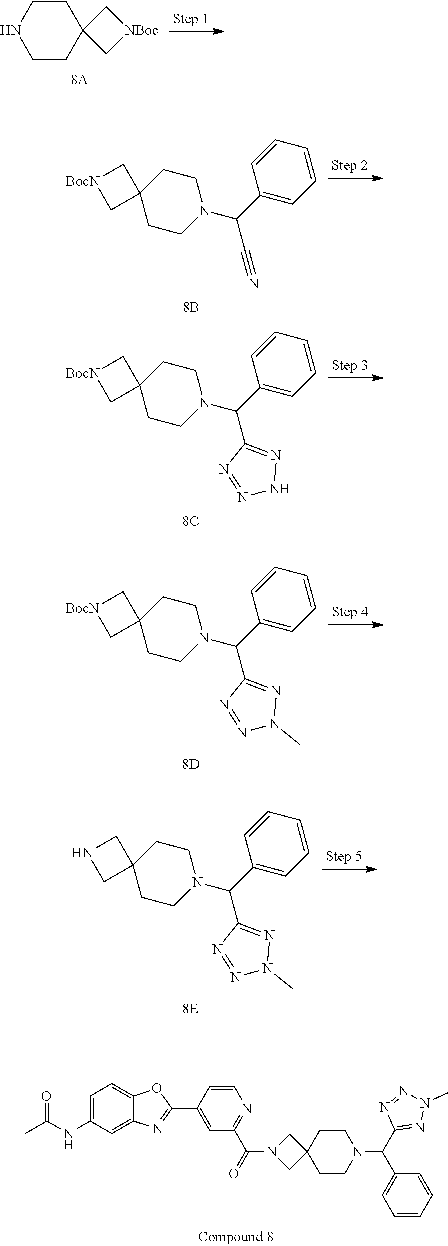 [SMILES: CC(C)(C)OC(=O)N1CC2(CCNCC2)C1 ; CC(C)(C)OC(=O)N1CC2(CCN(C(C#N)c3ccccc3)CC2)C1 ; CC(C)(C)OC(=O)N1CC2(CCN(C(c3ccccc3)c3nn[nH]n3)CC2)C1 ; Cn1nnc(C(c2ccccc2)N2CCC3(CC2)CN(C(=O)OC(C)(C)C)C3)n1 ; Cn1nnc(C(c2ccccc2)N2CCC3(CC2)CNC3)n1 ; CC(=O)Nc1ccc2oc(-c3ccnc(C(=O)N4CC5(CCN(C(c6ccccc6)c6nnn(C)n6)CC5)C4)c3)nc2c1]

Step 1:

### tert-butyl-7-(cyano(phenyl)methyl)-2,7-diazaspiro[3.5]nonane-2-carboxylate (8B)

Under nitrogen protection, acetonitrile (50 mL), 2-tert-butoxycarbonyl-2,7-diazaspiro[3.5]nonane 8A (4.27 g, 18.85 mmol), trimethylsilyl cyanide (1.87 g, 18.85 mmol) and iodine (0.51 g, 2.0 mmol) were successively added to compound benzaldehyde (2.0 g, 18.85 mmol). The reaction solution was stirred at room temperature for 15 hours and saturated aqueous sodium carbonate solution (50 mL) was added. The residue was extracted with ethyl acetate (100 mL×2). The combined organic phase was dried over anhydrous sodium sulfate and concentrated under reduced pressure, and then the residue was separated and purified by silica gel column chromatography (PE/EA=3:1) to obtain 8B (3 g, 46.6%).

¹H NMR (400 MHz, CDCl₃) δ 7.58-7.55 (m, 2H), 7.44-7.37 (m, 3H), 4.92 (s, 1H), 3.64 (s, 4H), 2.56 (s, 4H), 1.90-1.79 (m, 4H), 1.43 (s, 9H).

LC-MS (ESI): m/z=342.3 [M+H]⁺.

Step 2:

### tert-butyl-7-(phenyl(2H-tetrazol-5-yl)methyl)-2,7-diazaspiro[3.5]nonane-2-carboxylate (8C)

Under nitrogen protection, DMF (8 mL), toluene (24 mL), and azidotributyltin (2.34 g, 7.05 mmol) were successively added to compound 8B (1.6 g, 4.7 mmol), and the reaction solution was warmed to 130° C. and stirred for 30 h. The reaction solution was cooled to room temperature and then washed with saturated aqueous potassium carbonate solution (30 mL×2). The combined aqueous phase was adjusted to pH=2-3 with 6 N hydrochloric acid, and the residue was extracted with ethyl acetate (100 mL×2). The combined organic phase was dried over anhydrous sodium sulfate and concentrated under reduced pressure to obtain 8C (1.7 g, 77.7%).

LC-MS (ESI): m/z=385.3 [M+H]⁺.

Step 3:

### tert-butyl-7-((2-methyl-2H-tetrazol-5-yl)(phenyl)methyl)-2,7-diazaspiro[3.5]nonane-2-carboxylate (8D)

Acetonitrile (10 mL), potassium carbonate (2.0 g, 16.0 mmol) and iodomethane (1.55 g, 10.9 mmol) were successively added to compound 8C (1.4 g, 3.64 mmol). The reaction solution was stirred at room temperature for 3 hours and filtered. Water (100 mL) was added to the filtrate. The residue was extracted with ethyl acetate (100 mL×2). The combined organic phase was dried over anhydrous sodium sulfate and concentrated under reduced pressure, and then the residue was separated and purified by silica gel column chromatography (PE/EA=1:1) to obtain 8D (140 mg, 11.7%).

¹H NMR (400 MHz, CDCl₃) δ7.77-7.59 (m, 2H), 7.36-7.28 (m, 1H), 7.23-7.13 (m, 1H), 7.11-7.03 (m, 1H), 5.44 (s, 1H), 4.33 (s, 3H), 3.69-3.32 (m, 4H), 2.55-2.49 (m, 2H), 2.45-2.04 (m, 2H), 1.62-1.58 (m, 2H), 1.43 (s, 9H), 1.33-1.24 (m, 2H).

LC-MS (ESI): m/z=399.3[M+H]⁺.

Step 4:

### 7-((2-methyl-2H-tetrazol-5-yl)(phenyl)methyl)-2,7-diazaspiro[3.5]nonane (8E)

Methanol (5 mL) and concentrated hydrochloric acid (2 mL) were successively added to compound 8D (140 mg, 0.35 mmol). The reaction solution was stirred at room temperature for 2 h and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure, and then the residue was separated and purified by silica gel column chromatography (DCM:MeOH=30:1) to obtain intermediate 8E (100 mg, 95.4%).

LC-MS (ESI): m/z=299.3[M+H]⁺.

Step 5:

### N-(2-(2-(7-((2-methyl-2H-tetrazol-5-yl)(phenyl)methyl)-2,7-diazaspiro[3.5]nonane-2-carbonyl)pyridin-4-yl)benzo[d]oxazol-5-yl)acetamide (Compound 8)

Compound 8 (25 mg, 16.14%) was obtained with reference to the same method as that of Example 1, except replacing intermediate 2 with 7-((2-methyl-2H-tetrazol-5-yl)(phenyl)methyl)-2,7-diazaspiro[3.5]nonane (8E).

¹H NMR (400 MHz, CDCl₃) δ 8.81 (s, 1H), 8.73 (d, J=4.0 Hz, 1H), 8.15 (d, J=8.0 Hz, 1H), 8.02 (s, 1H), 7.63 (d, J=8.0 Hz, 2H), 7.55 (s, 3H), 7.41-7.36 (m, 3H), 5.59 (s, 1H), 4.38 (s, 3H), 3.22-2.96 (m, 2H), 2.85-2.51 (m, 2H), 2.22 (s, 3H), 2.08 (s, 4H), 2.04 (s, 3H).

LC-MS (ESI): m/z=578.3 [M+H]⁺.

### Example 9

### N-(2-(2-(4-((2-fluorophenyl)(2-methyl-2H-tetrazol-5-yl)methyl)piperazine-1-carbonyl)pyridin-4-yl)benzo[d]oxazol-5-yl)acetamide

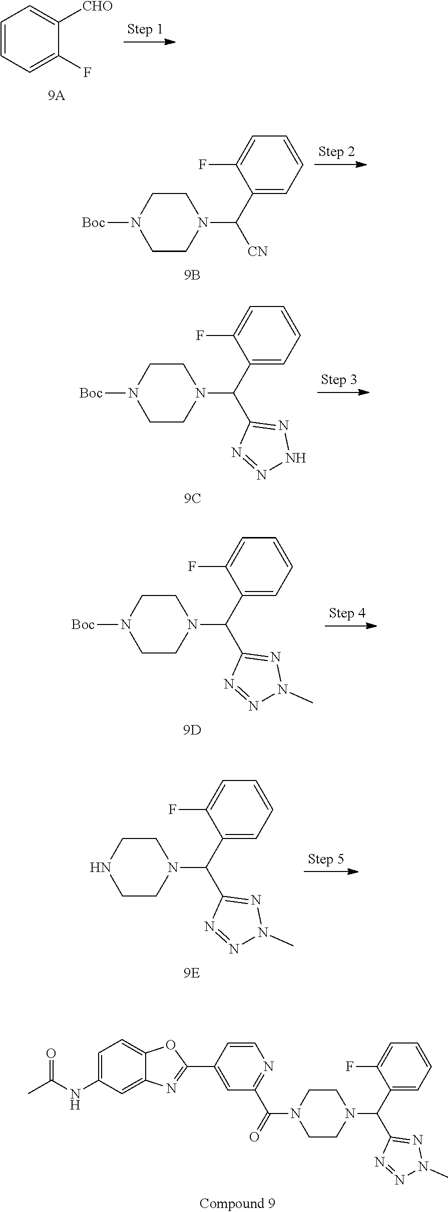 [SMILES: O=Cc1ccccc1F ; CC(C)(C)OC(=O)N1CCN(C(C#N)c2ccccc2F)CC1 ; CC(C)(C)OC(=O)N1CCN(C(c2nn[nH]n2)c2ccccc2F)CC1 ; Cn1nnc(C(c2ccccc2F)N2CCN(C(=O)OC(C)(C)C)CC2)n1 ; Cn1nnc(C(c2ccccc2F)N2CCNCC2)n1 ; CC(=O)Nc1ccc2oc(-c3ccnc(C(=O)N4CCN(C(c5nnn(C)n5)c5ccccc5F)CC4)c3)nc2c1]

Step 1:

### tert-butyl-4-(cyano(2-fluorophenyl)methyl)piperazine-1-carboxylate (9B)

Under nitrogen protection, acetonitrile (50 mL), 1-Boc-piperazine (5.0 g, 26.88 mmol), trimethylsilyl cyanide (3.2 g, 32.26 mmol) and iodine (0.68 g, 2.7 mmol) were successively added to compound 9A (2.9 g, 26.88 mmol). The reaction solution was stirred at room temperature for 15 h, and saturated aqueous sodium carbonate solution (50 mL) was added. The residue was extracted with ethyl acetate (100 mL×2). The combined organic phase was dried over anhydrous sodium sulfate and concentrated under reduced pressure, and then the residue was separated and purified by silica gel column chromatography (PE/EA=4:1) to obtain 9B (4.0 g, 50%).

LC-MS (ESI): m/z=246.2 [M+H]⁺.

Step 2:

### tert-butyl-4-((2-fluorophenyl)(2H-tetrazol-5-yl)methyl)piperazine-1-carboxylate (9C)

Under nitrogen protection, isopropanol (15 mL), sodium azide (1.95 g, 30 mmol) and zinc bromide (1.13 g, 5 mmol) were successively added to compound 9B (3.2 g, 10 mmol). The reaction solution was warmed to 80° C. and stirred for 30 h. The reaction solution was cooled to room temperature and then washed with saturated aqueous potassium carbonate solution (30 mL×2). The combined aqueous phase was adjusted to pH=2-3 with 6 N hydrochloric acid, and the residue was extracted with ethyl acetate (100 mL×2). The combined organic phase was dried over anhydrous sodium sulfate and concentrated under reduced pressure to obtain 9C (2.1 g, 57.8%).

LC-MS (ESI): m/z=363.3 [M+H]⁺.

Step 3:

### tert-butyl-4-((2-fluorophenyl)(2-methyl-2H-tetrazol-5-yl)methyl)piperazine-1-carboxylate (9D)

Acetonitrile (10 mL), and trimethylsilyldiazomethane (1.7 g, 12.0 mmol) were successively added to compound 9C (2.1 g, 5.79 mmol). The reaction solution was stirred at room temperature for 24 hours and filtered. Water (100 mL) was added to the filtrate. The residue was extracted with ethyl acetate (100 mL×2). The combined organic phase was dried over anhydrous sodium sulfate and concentrated under reduced pressure, and then the residue was separated and purified by silica gel column chromatography (PE/EA=1:1) to obtain 9D (300 mg, 13.75%).

¹H NMR (400 MHz, CDCl₃) δ 7.67-7.59 (m, 1H), 7.36-7.30 (m, 1H), 7.23-7.03 (m, 2H), 5.44 (s, 1H), 4.34 (s, 3H), 3.53-3.41 (m, 4H), 2.66-2.36 (m, 4H), 1.43 (s, 9H).

LC-MS (ESI): m/z=377.3[M+H]⁺.

Step 4:

### 1-((2-fluorophenyl)(2-methyl-2H-tetrazol-5-yl)methyl)piperazine (9E)

Dichloromethane (5 mL) and trifluoroacetic acid (2 mL) were successively added to compound 9D (170 mg, 0.47 mmol). The reaction solution was stirred at room temperature for 2 h and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure, and then the residue was separated and purified by silica gel column chromatography (DCM:MeOH=30:1) to obtain 9E (110 mg, 90%).

LC-MS (ESI): m/z=277.2[M+H]⁺.

Step 5:

### N-(2-(2-(4-((2-fluorophenyl)(2-methyl-2H-tetrazol-5-yl)methyl)piperazine-1-carbonyl)pyridin-4-yl)benzo[d]oxazol-5-yl)acetamide (Compound 9)

Under nitrogen, dichloromethane (10 mL), DIPEA (46 mg, 0.36 mmol), HATU (102 mg, 0.27 mmol), and 9E (50 mg, 0.18 mmol) were successively added to intermediate 1 (54 mg, 0.18 mmol) at room temperature, and the mixture was stirred at room temperature for 3 h. saturated aqueous sodium bicarbonate solution (30 mL) was added to the reaction solution. The mixture was extracted with dichloromethane (30 mL×1) and allowed to stand for phase separation. The aqueous phase was washed with dichloromethane (100 mL). The combined organic phase was dried over anhydrous sodium sulfate and concentrated under reduced pressure to obtain a crude product, which was separated and purified by silica gel column chromatography (DCM:MeOH=20:1) to obtain compound 9 (50 mg, 49.74%).

¹H NMR (400 MHz, CDCl₃) δ 8.72 (d, J=8.0 Hz, 1H), 8.35 (s, 1H), 8.09 (d, J=8.0 Hz, 1H), 7.99 (s, 1H), 7.85 (s, 1H), 7.51 (s, 3H), 7.34-7.31 (m, 1H), 7.22-7.18 (m, 1H), 7.11-7.06 (m, 1H), 5.58 (s, 1H), 4.36 (s, 3H), 3.98 (s, 2H), 3.79 (s, 2H), 2.81-2.63 (m, 4H), 2.22 (s, 3H).

LC-MS (ESI): m/z=556.2 [M+H]⁺.

### Example 10

### N-(2-(2-(6-((2-methyl-2H-tetrazol-5-yl)(phenyl)methyl)-2,6-diazaspiro[3.3]heptane-2-carbonyl)pyridin-4-yl)benzo[d]oxazol-5-yl)acetamide

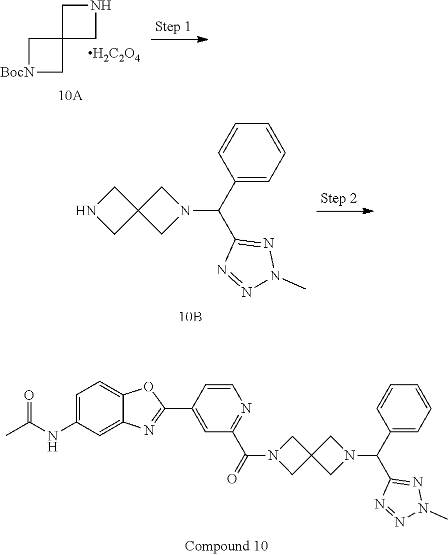

Step 1:

### 2-((2-methyl-2H-tetrazol-5-yl)(phenyl)methyl)-2,6-diazaspiro[3.3]heptane (10B)

Benzaldehyde and 10A were used as starting materials to obtain 10B according to the synthetic step of intermediate 2.

LC-MS (ESI): m/z=271.2[M+H]⁺.

Step 2:

### N-(2-(2-(6-((2-methyl-2H-tetrazol-5-yl)(phenyl)methyl)-2,6-diazaspiro[3.3]heptane-2-carbonyl)pyridin-4-yl)benzo[d]oxazol-5-yl)acetamide (Compound 10)

10B and intermediate 1 were used as raw materials to obtain compound 10 according to the synthetic step of compound 1.

¹H NMR (400 MHz, CDCl₃) δ 8.79 (s, 1H), 8.74-8.73 (m, 1H), 8.14-8.12 (m, 1H), 8.02 (s, 1H), 7.54-7.50 (m, 5H), 7.37-7.29 (m, 3H), 4.82 (s, 2H), 4.80 (s, 1H), 4.33 (s, 2H), 4.30 (s, 3H), 3.47-3.42 (m, 4H), 2.22 (s, 3H).

LC-MS (ESI): m/z=550.2 [M+H]⁺.

### Example 11

### N-(2-(2-(4-((3-fluorophenyl)(2-methyl-2H-tetrazol-5-yl)methyl)piperazine-1-carbonyl)pyridin-4-yl)benzo[d]oxazol-5-yl)acetamide

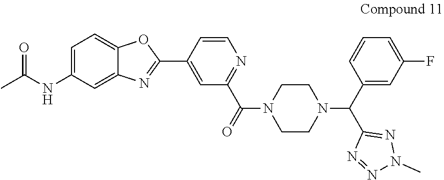 [SMILES: CC(=O)Nc1ccc2oc(-c3ccnc(C(=O)N4CCN(C(c5cccc(F)c5)c5nnn(C)n5)CC4)c3)nc2c1]

M-fluorobenzaldehyde was used as a starting material to obtain compound 11 according to the synthetic step of compound 1.

¹H NMR (400 MHz, CDCl₃) δ 8.72-8.71 (m, 1H), 8.34 (s, 1H), 8.08-8.07 (m, 1H), 7.99 (s, 1H), 7.85 (s, 1H), 7.51-7.48 (m, 2H), 7.36-7.32 (m, 2H), 7.05-7.00 (m, 1H), 5.20 (s, 1H), 4.38 (s, 3H), 4.05-3.91 (m, 2H), 3.78-3.74 (m, 2H), 2.89-2.55 (m, 4H), 2.22 (s, 3H).

LC-MS (ESI): m/z=556.2 [M+H]⁺.

### Example 12

### N-(2-(2-(4-((4-fluorophenyl)(2-methyl-2H-tetrazol-5-yl)methyl)piperazine-1-carbonyl)pyridin-4-yl)benzo[d]oxazol-5-yl)acetamide

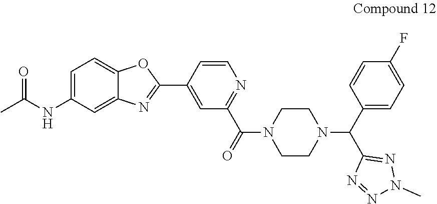 [SMILES: CC(=O)Nc1ccc2oc(-c3ccnc(C(=O)N4CCN(C(c5ccc(F)cc5)c5nnn(C)n5)CC4)c3)nc2c1]

P-fluorobenzaldehyde was used as a starting material to obtain compound 12 according to the synthetic step of compound 1.

¹H NMR (400 MHz, CDCl₃) δ 8.72-8.71 (m, 1H), 8.51 (s, 1H), 8.21-8.20 (m, 1H), 8.06 (s, 1H), 8.02-7.99 (m, 2H), 7.56 (s, 2H), 7.34 (s, 1H), 7.20-7.16 (m, 2H), 5.93 (s, 1H), 4.47 (s, 3H), 4.30-4.23 (m, 2H), 3.74-3.50 (m, 2H), 3.35-3.05 (m, 4H), 2.24 (s, 3H).

LC-MS (ESI): m/z=556.2 [M+H]⁺.

### Example 13

### N-(2-(2-(2-((2-methyl-2H-tetrazol-5-yl)(phenyl)methyl)-2,7-diazaspiro[3.5]nonane-7-carbonyl)pyridin-4-yl)benzo[d]oxazol-5-yl)acetamide

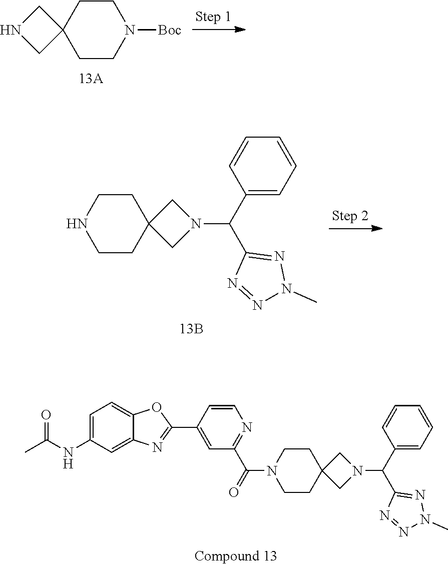 [SMILES: CC(C)(C)OC(=O)N1CCC2(CC1)CNC2 ; Cn1nnc(C(c2ccccc2)N2CC3(CCNCC3)C2)n1 ; CC(=O)Nc1ccc2oc(-c3ccnc(C(=O)N4CCC5(CC4)CN(C(c4ccccc4)c4nnn(C)n4)C5)c3)nc2c1]

Step 1:

### 2-((2-methyl-2H-tetrazol-5-yl)(phenyl)methyl)-2,7-diazaspiro[3.5]nonane (13B)

Benzaldehyde and 13A were used as starting materials to obtain 13B according to the synthetic step of intermediate 2.

LC-MS (ESI): m/z=299.3[M+H]⁺.

Step 2:

### N-(2-(2-(2-((2-methyl-2H-tetrazol-5-yl)(phenyl)methyl)-2,7-diazaspiro[3.5]nonane-7-carbonyl)pyridin-4-yl)benzo[d]oxazol-5-yl)acetamide (Compound 13)

13B and intermediate 1 were used as raw materials to obtain compound 13 according to the synthetic step of compound 1.

¹H NMR (400 MHz, CDCl₃) δ 8.74-8.73 (m, 1H), 8.25 (s, 1H), 8.07-8.06 (m, 1H), 7.97 (s, 1H), 7.71 (s, 1H), 7.53-7.45 (m, 3H), 7.42-7.33 (m, 3H), 5.30 (s, 1H), 4.32 (s, 3H), 4.15-4.09 (m, 2H), 3.74-3.69 (m, 2H), 3.47-3.40 (m, 2H), 3.20-3.12 (m, 2H), 2.21 (s, 4H), 2.04 (s, 3H).

LC-MS (ESI): m/z=578.3 [M+H]⁺.

### Example 14

### N-(2-(2-((1S,4S)-5-((2-methyl-2H-tetrazol-5-yl)(phenyl)methyl)-2,5-diazabicyclo[2.2.1]heptane-2-carbonyl)pyridin-4-yl)benzo[d]oxazol-5-yl)acetamide

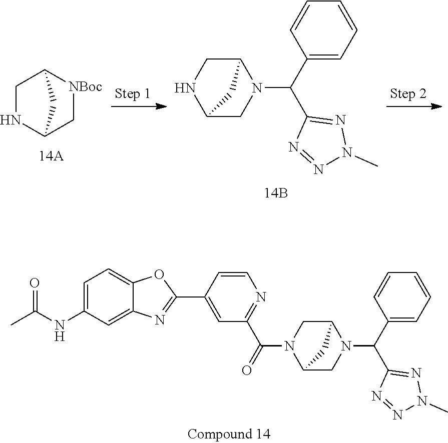

Step 1:

### (1S,4S)-2-((2-methyl-2H-tetrazol-5-yl)(phenyl)methyl)-2,5-diazabicyclo[2.2.1]heptane (14B)

Benzaldehyde and 14A were used as starting materials to obtain 14B according to the synthetic step of intermediate 2.

LC-MS (ESI): m/z=271.2[M+H]⁺.

Step 2:

### N-(2-(2-((1S,4S)-5-((2-methyl-2H-tetrazol-5-yl)(phenyl)methyl)-2,5-diazabicyclo[2.2.1]heptane-2-carbonyl)pyridin-4-yl)benzo[d]oxazol-5-yl)acetamide (compound 14)

14B and intermediate 1 were used as raw materials to obtain compound 14 according to the synthetic step of compound 1.

¹H NMR (400 MHz, DMSO-d6) δ 10.17 (s, 1H), 8.87-8.81 (m, 1H), 8.55-8.37 (m, 1H), 8.28-8.13 (m, 2H), 7.84-7.75 (m, 1H), 7.66-7.49 (m, 3H), 7.36-7.26 (m, 3H), 4.89-4.85 (m, 1H), 4.30 (d, 3H), 3.95-3.91 (m, 1H), 3.75-3.65 (m, 1H), 3.39-3.35 (m, 2H), 2.94-2.74 (m, 1H), 2.67-2.62 (m, 1H), 2.09 (s, 3H), 1.92-1.87 (m, 1H), 1.76-1.72 (m, 1H).

LC-MS (ESI): m/z=550.3 [M+H]⁺.

### Example 15

### N-(2-(4-cyano-3-(4-((2-methyl-2H-tetrazol-5-yl)(phenyl)methyl)piperazine-1-carbonyl)phenyl)benzo[d]oxazol-5-yl)acetamide

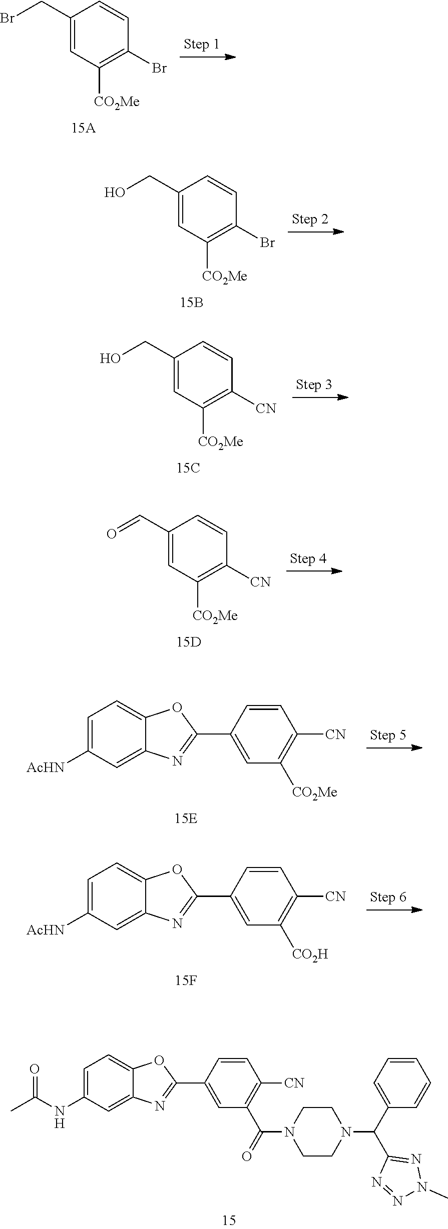

Step 1:

### Methyl 2-bromo-5-(hydroxymethyl)benzoate (15B)

1,4-dioxane (10 mL), water (10 mL), tetrabutylammonium bromide (0.8 g, 2.60 mmol) and sodium bicarbonate (12.00 g, 140.00 mmol) were successively added to compound 15A (4.0 g, 13.00 mmol). The reaction solution was stirred at 70° C. for 5 hours, cooled to room temperature and adjusted to pH=3-4 by adding dilute hydrochloric acid (2 mol/L). The resulting reaction solution was extracted with ethyl acetate (20 mL×2). The combined organic phase was dried over anhydrous sodium sulfate and filtered. The filtrate was concentrated under reduced pressure, and then the residue was separated and purified by silica gel column chromatography (PE/EA=3:1) to obtain 15B (3.0 g, 98%).

¹H NMR (400 MHz, CDCl₃) δ 7.76 (d, 1H), 7.62 (d, 1H), 7.32 (dd, 1H), 4.67 (s, 2H), 3.92 (s, 3H).

Step 2:

### Methyl 2-cyano-5-(hydroxymethyl)benzoate (15C)

Under nitrogen protection, N,N-dimethylformamide (5 mL), copper cyanide (0.20 g, 2.00 mmol) and potassium iodide (0.04 g, 0.20 mmol) were successively added to compound 15B (0.30 g, 1.00 mmol). The reaction solution was stirred at 130° C. for 7 hours and cooled to room temperature. Water (150 mL) was added, and the resulting solution was extracted with ethyl acetate (150 mL×2). The organic layer was combined, washed twice with 200 mL of saturated aqueous sodium chloride solution. The organic phase was dried over anhydrous sodium sulfate and filtered. The filtrate was concentrated under reduced pressure, and then the residue was separated and purified by silica gel column chromatography (PE/EA=2:1) to obtain 15C (0.10 g, 40%).

¹H NMR (400 MHz, MeOD) δ 8.15 (d, 1H), 7.85 (d, 1H), 7.75-7.70 (m, 1H), 4.73 (s, 2H), 3.98 (s, 3H).

LC-MS (ESI): m/z=192.2[M+H]⁺.

Step 3:

### Methyl 2-cyano-5-formylbenzoate (15D)

Toluene (50 mL) and manganese dioxide (4.5 g, 52.00 mmol) were successively added to compound 15C (2.0 g, 10.00 mmol). The reaction was stirred for 0.5 hours at 100° C., cooled to room temperature and filtered. The filtrate was concentrated under reduced pressure to obtain 15D (1.6 g, 81%), which was directly used in the next reaction.

LC-MS (ESI): m/z=190.1[M+H]⁺.

Step 4:

### Methyl 5-(5-acetamidobenzo[d]oxazol-2-yl)-2-cyanobenzoate (15E)

Methanol (50 mL) and 1c (1.7 g, 10.00 mmol) were successively added to compound 15D (1.6 g, 8.50 mmol). The reaction solution was stirred for 2 hours at 60° C., cooled to room temperature and concentrated to dryness. Dichloromethane (50 mL) and 2,3-dichloro-5,6-dicyano-1,4-benzoquinone (2.9 g, 13.00 mmol) were added, and the mixture was reacted at room temperature overnight. The reaction solution was filtered, and the filtrate was concentrated under reduced pressure to obtain 15E (2.0 g, 71%), which was directly used in the next reaction.

LC-MS (ESI): m/z=336.1[M+H]⁺.

Step 5:

### 5-(5-acetamidobenzo[d]oxazol-2-yl)-2-cyanobenzoic Acid (15F)

Methanol (5 mL), water (5 mL), and lithium hydroxide (0.57 g, 24.00 mmol) were successively added to compound 15E (2.0 g, 6.00 mmol). The reaction solution was stirred at room temperature for 2 hours and adjusted to pH=3-4 by adding dilute hydrochloric acid (2 mol/L). The resulting reaction solution was extracted with ethyl acetate (20 mL×2). The combined organic phase was dried over anhydrous sodium sulfate and filtered. The filtrate was concentrated under reduced pressure, and then the residue was separated and purified by silica gel column chromatography (PE/EA=1:1) to obtain 15F (60 mg, 3%).

LC-MS (ESI): m/z=322.1[M+H]⁺.

Step 6:

### N-(2-(4-cyano-3-(4-((2-methyl-2H-tetrazol-5-yl)(phenyl)methyl)piperazine-1-carbonyl)phenyl)benzo[d]oxazol-5-yl)acetamide (compound 15)

N,N-dimethylformamide (5 mL), HATU (106 mg, 0.28 mmol), DIPEA (0.22 g, 1.87 mmol) and intermediate 2 (72 mg, 0.28 mmol) were successively added to compound 15F (60 mg, 0.19 mmol). The reaction solution was stirred at room temperature overnight, and water (20 mL) was added. The resulting reaction solution was extracted with ethyl acetate (20 mL×2). The combined organic phase was washed with water (15 mL×2), dried over anhydrous sodium sulfate and filtered. The filtrate was concentrated under reduced pressure, and then the residue was separated and purified by silica gel column chromatography (DCM/MeOH=20:1) to obtain compound 15 (2.5 mg, 2.4%).

¹H NMR (400 MHz, CDCl₃) δ 8.35 (d, 1H), 8.26 (s, 1H), 7.98 (s, 1H), 7.83 (d, 1H), 7.75-7.58 (m, 2H), 7.52 (d, 2H), 7.50-7.38 (m, 3H), 5.67 (s, 1H), 4.40 (s, 3H), 4.15 (d, 2H), 3.62 (s, 2H), 3.29 (d, 2H), 2.96 (s, 2H), 2.24 (s, 3H).

LC-MS (ESI): m/z=562.1[M+H]⁺.

### Example 16

### N-(2-(2-((R)-3-methyl-4-((R/S)-(2-methyl-2H-tetrazol-5-yl)(phenyl)methyl)piperazine-1-carbonyl)pyridin-4-yl)benzo[d]oxazol-5-yl)acetamide (Compound 16a and Compound 16b)

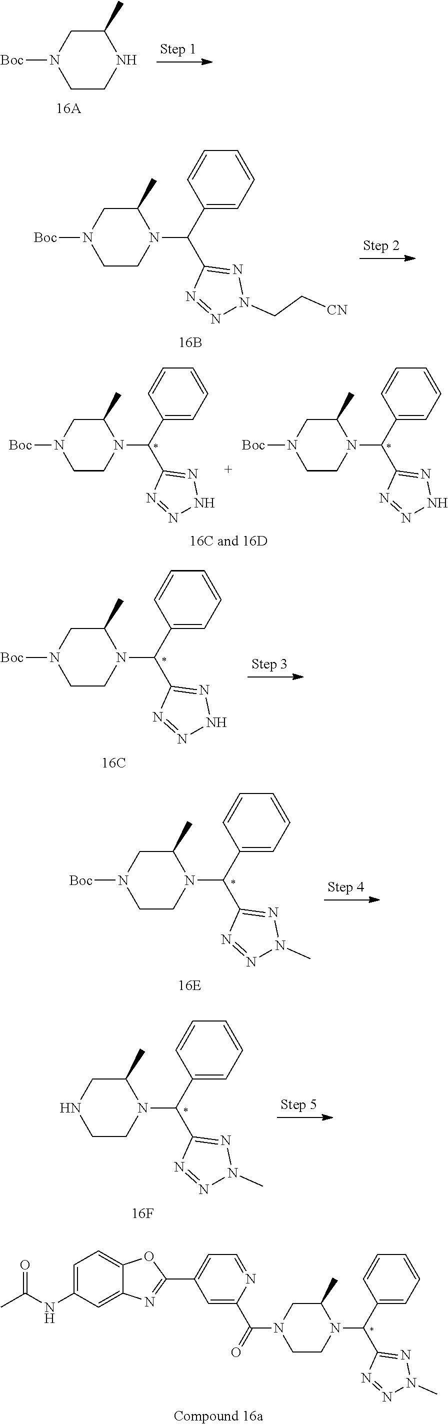

Step 1:

### tert-butyl (3R)-4-((2-(2-cyanoethyl)-2H-tetrazol-5-yl)(phenyl)methyl)-3-methylpiperazine-1-carboxylate (16B)

Methanol (20 mL) and benzaldehyde (1.14 g, 10.7 mmol) were successively added to compound 16A (2.14 g, 10.7 mmol), and the mixture was stirred at room temperature until the system was clear. Then azidotrimethylsilane (1.86 g, 16.05 mmol) and 3-isocyanopropanenitrile (1.3 g, 16.05 mmol) were successively added. The reaction solution was stirred at room temperature for 2 hours and concentrated under reduced pressure to obtain 16B (4.0 g, 92%).

Step 2:

### tert-butyl (3R)-3-methyl-4-(phenyl(2H-tetrazol-5-yl)methyl)piperazine-1-carboxylate (16C, Isomer 1)

### tert-butyl (3R)-3-methyl-4-(phenyl(2H-tetrazol-5-yl)methyl)piperazine-1-carboxylate (16D, Isomer 2)

Tetrahydrofuran (32 mL), water (8 mL), and lithium hydroxide (1.3 g, 29.4 mmol) were successively added to compound 16B (4.0 g, 9.8 mmol), and the mixture was stirred at room temperature for 1 hour and adjusted to pH 5-6 with saturated aqueous sodium carbonate solution. The aqueous phase was extracted with ethyl acetate (100 mL×2). The combined organic phase was dried over anhydrous sodium sulfate, concentrated under reduced pressure and subjected to prep-HPLC to obtain 16C (1.0 g, 29%, isomer 1) and 16D (316 mg, 9.2%, isomer 2). Preparation method: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 1% TFA); gradient elution: the content of mobile phase A rises from 20% to 60%; flow rate: 12 ml/min; elution time: 20 min; retention time: 14.4 min for 16C and 15.3 min for 16D.

LC-MS (ESI): m/z=359.3 [M+H]⁺.

Step 3:

### (3R)-tert-butyl-3-methyl-4-((2-methyl-2H-tetrazol-5-yl)(phenyl)methyl)piperazine-1-carboxylate (16E)

At room temperature, tetrahydrofuran (4 mL) and methanol (1 mL) were added to compound 16C (350 mg, 0.98 mmol), and the mixture was stirred to dissolution and clarification; and then trimethylsilyldiazomethane (223 mg, 1.96 mmol) was added dropwise, and the resulting mixture was stirred at room temperature for 30 minutes and diluted by adding water (50 mL). The residue was extracted with ethyl acetate (50 mL×2). The combined organic phase was dried over anhydrous sodium sulfate and concentrated under reduced pressure, and then the residue was separated and purified by silica gel column chromatography (PE:EA=1:1) to obtain 16E (200 mg, 55%).

LC-MS (ESI): m/z=373.1 [M+H]⁺.

Step 4:

### (2R)-2-methyl-1-((2-methyl-2H-tetrazol-5-yl)(phenyl)methyl)piperazine (16F)

Methanol (5 mL) and concentrated hydrochloric acid (2 mL) were successively added to compound 16E (200 mg, 0.54 mmol). The reaction solution was stirred at room temperature for 2 hours and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure to obtain compound 16F (130 mg, 89%).

LC-MS (ESI): m/z=273.1 [M+H]⁺.

Step 5:

### N-(2-(2-((3R)-3-methyl-4-((2-methyl-2H-tetrazol-5-yl)(phenyl)methyl)piperazine-1-carbonyl)pyridin-4-yl)benzo[d]oxazol-5-yl)acetamide (Compound 16a)

Intermediate 1 (69 mg, 0.23 mmol) was dissolved in N,N-dimethylformamide (2 mL), and the mixture was cooled to 0° C.; HATU (100 mg, 0.28 mmol) and DIPEA (86 mg, 0.69 mmol) were added; and then compound 16F (60 mg, 0.23 mmol) was added. After the addition, the mixture was stirred for 1 hour while the temperature was controlled at 0° C., and then ice water (10 mL) was added. The resulting solution was extracted twice with dichloromethane (50 mL×2). The organic phase was combined, dried over anhydrous sodium sulfate and concentrated under reduced pressure, and then the residue was separated by column chromatography (DCM:MeOH=20:1) to obtain compound 16a (50 mg, 41%, a compound in a single configuration).

¹H NMR (400 MHz, CD₃OD) δ 8.78-8.77 (m, 1H), 8.30 (s, 1H), 8.20-8.17 (m, 2H), 7.68-7.66 (m, 1H), 7.57-7.48 (m, 3H), 7.39-7.27 (m, 3H), 5.77-5.75 (d, 1H), 4.42-4.40 (d, 3H), 4.11-4.04 (m, 1H), 3.73-3.59 (m, 1H), 3.51-3.41 (m, 2H), 2.98-2.83 (m, 1H), 2.72-2.60 (m, 2H), 2.16 (s, 1H), 1.36-1.34 (d, 2H), 1.19-1.17 (d, 1H).

LC-MS (ESI): m/z=552.3 [M+H]⁺.

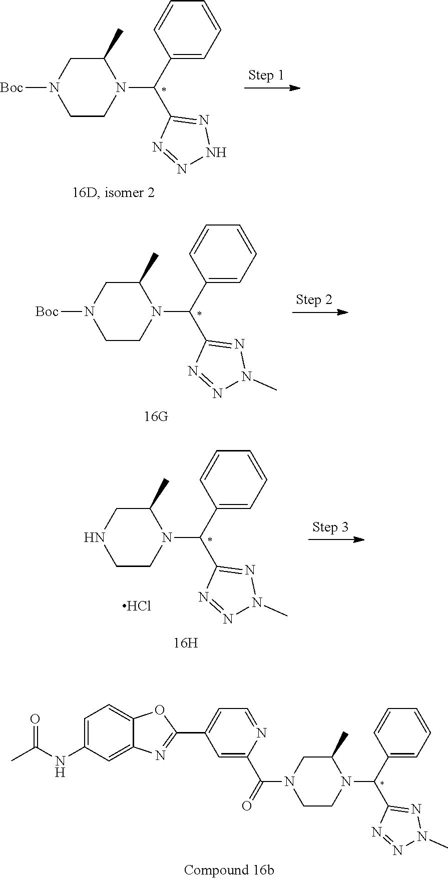 [SMILES: C[C@@H]1CN(C(=O)OC(C)(C)C)CCN1C(c1ccccc1)c1nn[nH]n1 ; C[C@@H]1CN(C(=O)OC(C)(C)C)CCN1C(c1ccccc1)c1nnn(C)n1 ; C[C@@H]1CNCCN1C(c1ccccc1)c1nnn(C)n1 ; Cl ; CC(=O)Nc1ccc2oc(-c3ccnc(C(=O)N4CCN(C(c5ccccc5)c5nnn(C)n5)[C@H](C)C4)c3)nc2c1]

Step 1:

### tert-butyl (3R)-3-methyl-4-((2-methyl-2H-tetrazol-5-yl)(phenyl)methyl)piperazine-1-carboxylate (16G)

Tetrahydrofuran (4 mL) and methanol (1 mL) were successively added to compound 16D (316 mg, 0.88 mmol), and then trimethylsilyldiazomethane (200 mg, 1.76 mmol) was added dropwise. The reaction solution was stirred at room temperature for 15 minutes, and then trimethylsilyldiazomethane (200 mg, 1.76 mmol) was added dropwise. Water (30 mL) was added to the system, and the residue was extracted with ethyl acetate (30 mL×2). The combined organic phase was dried over anhydrous sodium sulfate and concentrated under reduced pressure, and then the residue was separated and purified by silica gel column chromatography (PE:EA=1:1) to obtain 16G (280 mg, 85%).

¹H NMR (400 MHz, CDCl₃) δ 7.63-7.61 (d, 2H), 7.35-7.26 (m, 3H), 5.26 (s, 1H), 4.30 (s, 3H), 3.75-3.50 (m, 1H), 3.45-3.25 (m, 3H), 2.86-2.77 (m, 2H), 2.25-2.15 (m, 1H), 1.42 (s, 9H), 1.05-1.03 (d, 3H).

LC-MS (ESI): m/z=373.3 [M+H]⁺.

Step 2:

### (R)-2-methyl-1-((2-methyl-2H-tetrazol-5-yl)(phenyl)methyl)piperazine (16H)

4 mol/L of a solution of hydrogen chloride in methanol (4 mL) was added to compound 16G (280 mg, 0.75 mmol). The reaction solution was stirred at room temperature for 2 hours and concentrated under reduced pressure to obtain the hydrochloride of compound 16H (200 mg, 87%).

LC-MS (ESI): m/z=273.3[M+H]⁺.

Step 3:

### N-(2-(2-((R)-3-methyl-4-((2-methyl-2H-tetrazol-5-yl)(phenyl)methyl)piperazine-1-carbonyl)pyridin-4-yl)benzo[d]oxazol-5-yl)acetamide (compound 16b)

Intermediate 1 (66 mg, 0.22 mmol) was dissolved in N,N-dimethylformamide (2 mL), and the mixture was cooled to 0° C.; HATU (84 mg, 0.22 mmol) and DIPEA (72 mg, 0.55 mmol) were added; and then the hydrochloride of compound 16H (50 mg, 0.18 mmol) was added. After the addition, the mixture was stirred for 1 hour while the temperature was controlled at 0° C., and then ice water (10 mL) was added. The resulting solution was extracted twice with dichloromethane (50 mL×2). The organic phase was combined, dried over anhydrous sodium sulfate and concentrated under reduced pressure, and then the residue was separated by column chromatography (DCM:MeOH=20:1) to obtain compound 16b (50 mg, 49%, a compound in a single configuration).

¹H NMR (400 MHz, CD₃OD) δ 8.78-8.77 (m, 1H), 8.28 (s, 1H), 8.20-8.17 (m, 2H), 7.68-7.66 (m, 1H), 7.64-7.52 (m, 3H), 7.37-7.26 (m, 3H), 5.31-5.30 (d, 1H), 4.36-4.31 (d, 3H), 4.16-3.98 (m, 1H), 3.66-3.43 (m, 3H), 3.02-2.86 (m, 3H), 2.44-2.27 (m, 1H), 2.16 (s, 3H), 1.17-1.16 (d, 2H), 1.02-1.01 (d, 1H).

LC-MS (ESI): m/z=552.3[M+H]⁺.

### Example 17

### N-(2-(2-(3-((2-(difluoromethyl)-2H-tetrazol-5-yl)(phenyl)methyl)-3,8-diazabicyclo[3.2.1]octane-8-carbonyl)pyridin-4-yl)benzo[d]oxazol-5-yl)acetamide

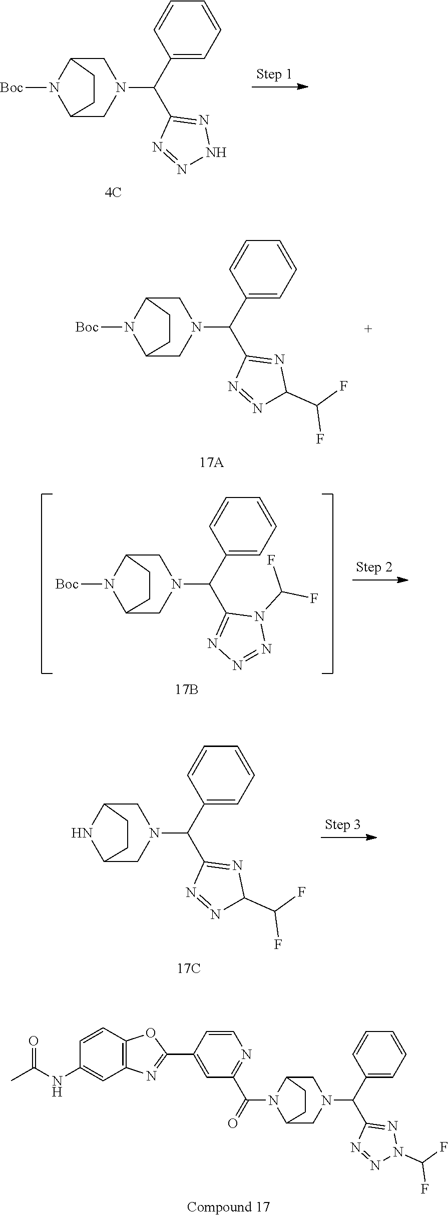

Step 1:

### tert-butyl-3-((2-(difluoromethyl)-2H-tetrazol-5-yl)(phenyl)methyl)-3,8-diazabicyclo[3.2.1]octane-8-carboxylate (17A)

### tert-butyl 3-((1-(difluoromethyl)-1H-tetrazol-5-yl)(phenyl)methyl)-3,8-diazabicyclo[3.2.1]octane-8-carboxylate (17B)

Acetonitrile (1.6 mL) was added to compound 4C (157 mg, 0.424 mmol), and the mixture was cooled to 0° C.; and then potassium hydroxide (473 mg, 8.48 mmol) and water (1.6 mL) were added. The reaction solution was stirred at 0° C. for 10 minutes, and diethyl bromofluorophosphate (225 mg, 0.848 mmol) was added. The reaction solution was stirred at 0° C. for 30 minutes. Water (100 mL) was added to the filtrate, and the residue was extracted with ethyl acetate (100 mL×2). The combined organic phase was dried over anhydrous sodium sulfate and concentrated under reduced pressure, and then the residue was separated and purified by silica gel column chromatography (developing solvent: PE:EA=2:1) to obtain 17A (80 mg, 45%, Rf value: 0.4) and 17B (75 mg, 42%, Rf value: 0.3).

LC-MS (ESI): m/z=421.2 [M+H]⁺.

Compound 17A: ¹H NMR (400 MHz, CDCl₃) δ 7.74-7.46 (t, 1H), 7.62-7.61 (d, 2H), 7.37-7.28 (m, 3H), 5.14 (s, 1H), 4.21-4.07 (m, 2H), 2.76-2.73 (m, 1H), 2.65-2.46 (m, 2H), 2.31 (s, 1H), 2.02-2.00 (m, 2H), 1.86-1.84 (m, 2H), 1.42 (s, 9H).

Compound 17B: ¹H NMR (400 MHz, CDCl₃) δ 7.92-7.64 (t, 1H), 7.46-7.43 (m, 2H), 7.40-7.36 (m, 3H), 5.27 (s, 1H), 4.20-4.10 (m, 2H), 2.84-2.43 (m, 4H), 2.00-1.87 (m, 4H), 1.41 (s, 9H).

Step 2:

### 3-((2-(difluoromethyl)-2H-tetrazol-5-yl)(phenyl)methyl)-3,8-diazabicyclo[3.2.1]octane (17C)

Methanol (5 mL) and concentrated hydrochloric acid (2 mL) were successively added to compound 17A (80 mg, 0.19 mmol). The reaction solution was stirred at room temperature for 2 hours and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure to obtain compound 17C (56 mg, 92%).

LC-MS (ESI): m/z=321.2 [M+H]⁺.

Step 4:

### N-(2-(2-(3-((2-(difluoromethyl)-2H-tetrazol-5-yl)(phenyl)methyl)-3,8-diazabicyclo[3.2.1]octane-8-carbonyl)pyridin-4-yl)benzo[d]oxazol-5-yl)acetamide (Compound 17)

Intermediate 1 (55 mg, 0.185 mmol) was dissolved in N,N-dimethylformamide (2 mL), and the mixture was cooled to 0° C.; HATU (80 mg, 0.21 mmol) and DIPEA (68 mg, 0.525 mmol) were added; and then compound 17C (56 mg, 0.175 mmol) was added. After the addition, the mixture was stirred for 1 hour while the temperature was controlled at 0° C., and then ice water (10 mL) was added. The resulting solution was extracted twice with dichloromethane (50 mL×2). The organic phase was combined, dried over anhydrous sodium sulfate and concentrated under reduced pressure, and then the residue was separated by column chromatography (DCM:MeOH=20:1) to obtain compound 17 (50 mg, 48%).

¹H NMR (400 MHz, CD₃OD) δ 8.79-8.77 (m, 1H), 8.43 (s, 1H), 8.30-8.00 (m, 3H), 7.68-7.66 (m, 1H), 7.56-7.50 (m, 3H), 7.39-7.28 (m, 3H), 5.21-5.20 (m, 1H), 4.63 (m, 1H), 3.32-3.29 (m, 1H), 2.90-2.77 (m, 1H), 2.70-2.56 (m, 2H), 2.50-2.44 (m, 1H), 2.16 (s, 3H), 2.14-2.07 (m, 2H), 2.01-1.96 (m, 2H).

LC-MS (ESI): m/z=600.2[M+H]⁺.

### Example 18

### N-(2-(2-(3-((1-(difluoromethyl)-1H-tetrazol-5-yl)(phenyl)methyl)-3,8-diazabicyclo[3.2.1]octane-8-carbonyl)pyridin-4-yl)benzo[d]oxazol-5-yl)acetamide

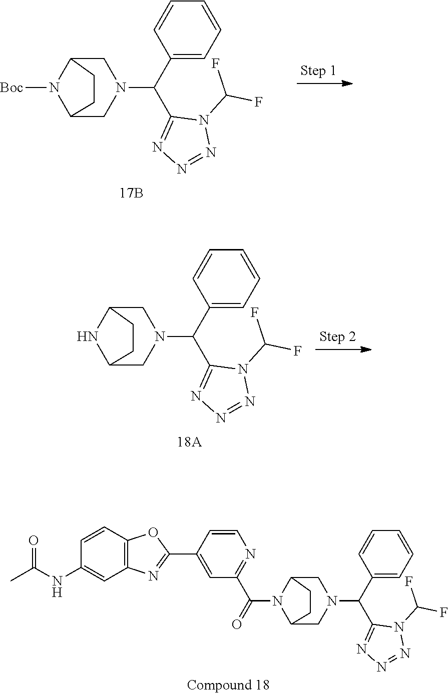 [SMILES: CC(C)(C)OC(=O)N1C2CCC1CN(C(c1ccccc1)c1nnnn1C(F)F)C2 ; FC(F)n1nnnc1C(c1ccccc1)N1CC2CCC(C1)N2 ; CC(=O)Nc1ccc2oc(-c3ccnc(C(=O)N4C5CCC4CN(C(c4ccccc4)c4nnnn4C(F)F)C5)c3)nc2c1]

Step 1:

### 3-((1-(difluoromethyl)-1H-tetrazol-5-yl)(phenyl)methyl)-3,8-diazabicyclo[3.2.1]octane (18A)

Methanol (5 mL) and concentrated hydrochloric acid (2 mL) were successively added to compound 17B (75 mg, 0.17 mmol). The reaction solution was stirred at room temperature for 2 hours and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure to obtain compound 18A (50 mg, 88%).

LC-MS (ESI): m/z=321.2 [M+H]⁺.

Step 2:

### N-(2-(2-(3-((1-(difluoromethyl)-1H-tetrazol-5-yl)(phenyl)methyl)-3,8-diazabicyclo[3.2.1]octane-8-carbonyl)pyridin-4-yl)benzo[d]oxazol-5-yl)acetamide (Compound 18)

Intermediate 1 (49 mg, 0.165 mmol) was dissolved in N,N-dimethylformamide (2 mL), and the mixture was cooled to 0° C.; HATU (72 mg, 0.187 mmol) and DIPEA (60 mg, 0.468 mmol) were added; and then compound 18A (50 mg, 0.156 mmol) was added. After the addition, the mixture was stirred for 1 hour while the temperature was controlled at 0° C., and then ice water (10 mL) was added. The resulting solution was extracted twice with dichloromethane (50 mL×2). The organic phase was combined, dried over anhydrous sodium sulfate and concentrated under reduced pressure, and then the residue was separated by column chromatography (DCM:MeOH=20:1) to obtain compound 18 (50 mg, 48%).

¹H NMR (400 MHz, DMSO-d₆) δ 10.16 (s, 1H), 8.82-8.79 (m, 1H), 8.58-8.42 (d, 1H), 8.31-8.20 (m, 1H), 8.23-8.22 (d, 1H), 8.16-8.14 (m, 1H), 7.80-7.77 (d, 1H), 7.60-7.56 (m, 1H), 7.47-7.38 (m, 5H), 5.62-5.60 (d, 1H), 4.70-4.67 (m, 2H), 2.81-2.71 (m, 1H), 2.67-2.55 (m, 2H), 2.42-2.38 (m, 1H), 2.09 (s, 3H), 1.96-1.81 (m, 4H).

LC-MS (ESI): m/z=600.2[M+H]⁺.

### Example 19

### 1-(2-(2-((1R,5S)-3-((2-methyl-2H-tetrazol-5-yl)(phenyl)methyl)-3,8-diazabicyclo[3.2.1]octane-8-carbonyl)pyridin-4-yl)benzo[d]oxazol-5-yl)pyrrolidin-2-one

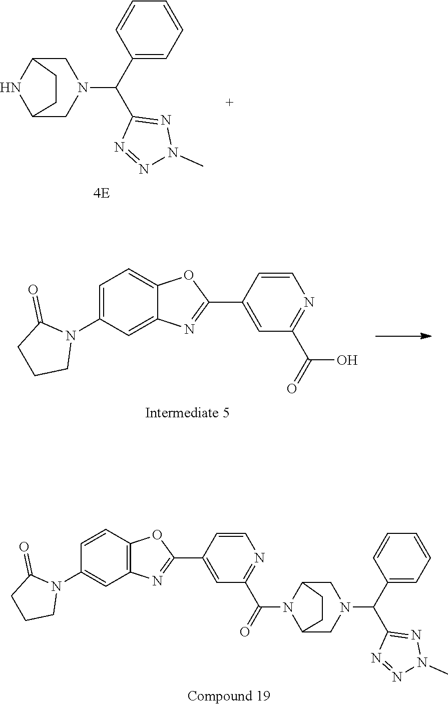 [SMILES: Cn1nnc(C(c2ccccc2)N2CC3CCC(C2)N3)n1 ; O=C(O)c1cc(-c2nc3cc(N4CCCC4=O)ccc3o2)ccn1 ; Cn1nnc(C(c2ccccc2)N2CC3CCC(C2)N3C(=O)c2cc(-c3nc4cc(N5CCCC5=O)ccc4o3)ccn2)n1]

Intermediate 5 (32.0 mg, 0.10 mmol) was dissolved in N,N-dimethylformamide (2 mL), and the mixture was cooled to 0° C.; HATU (35.5 mg, 0.12 mmol) and DIPEA (38.7 mg, 0.3 mmol) were added; and then compound 4E (30.0 mg, 0.11 mmol) was added. After the addition, the mixture was stirred for 1 hour while the temperature was controlled at 0° C., and then ice water (2 mL) was added. The resulting solution was extracted twice with dichloromethane (25 mL×2). The organic phase was combined, dried over anhydrous sodium sulfate and filtered. The filtrate was concentrated under reduced pressure, and then the residue was separated by column chromatography (eluent: EA/DCM=30%-70%) to obtain compound 19 (23 mg, 39%).

¹H NMR (400 MHz, CD₃OD) δ 8.76-8.74 (m, 1H), 8.40 (s, 1H), 8.14-8.13 (m, 1H), 8.01 (s, 1H), 7.74-7.68 (m, 2H), 7.50-7.48 (m, 2H), 7.35-7.24 (m, 3H), 5.03 (d, 1H), 4.75 (s, 1H), 4.68 (s, 1H), 4.35 (d, 3H), 4.00-3.96 (m, 2H), 2.87-2.41 (m, 6H), 2.21-1.90 (m, 6H).

LC-MS (ESI): m/z=590.2 [M+H]⁺.

### Example 20

### N-(2-(2-(3-((2-methyl-2H-tetrazol-5-yl)(phenyl)methyl)-3,6-diazabicyclo[3.1.1]heptane-6-carbonyl)pyridin-4-yl)benzo[d]oxazol-5-yl)acetamide

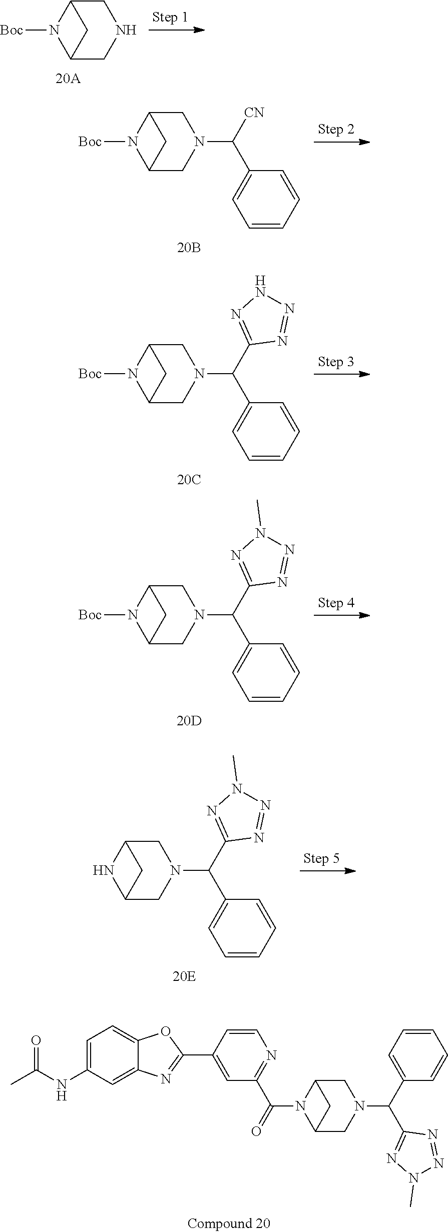

Step 1:

### tert-butyl 3-(cyano(phenyl)methyl)-3,6-diazabicyclo[3.1.1]heptane-6-carboxylate (20B)

Compound 20B (1.42 g, 90%) was obtained by using compound 20A (1.0 g, 5.0 mmol) as a starting material with reference to the same operating method as that of compound 1b.

LC-MS (ESI): m/z=314.1 [M+H]⁺.

Step 2:

### tert-butyl-3-(phenyl(2H-tetrazol-5-yl)methyl)-3,6-diazabicyclo[3.1.1]heptane-6-carboxylate (20C)

Compound 20C (270 mg, 17%) was obtained by using compound 20B (1.42 g, 4.5 mmol) as a starting material with reference to the same operating method as that of compound 1c.

LC-MS (ESI): m/z=357.1 [M+H]⁺.

Step 3:

### tert-butyl-3-((2-methyl-2H-tetrazol-5-yl)(phenyl)methyl)-3,6-diazabicyclo[3.1.1]heptane-6-carboxylate (20D)

Compound 20C (270 mg, 0.76 mmol) was dissolved in a mixed solvent of tetrahydrofuran (4 mL) and methanol (1 mL), and trimethylsilyldiazomethane (173.6 mg, 1.52 mmol, 2 mol/L n-hexane solution) was added. After the addition, the mixture was reacted at room temperature for 1 hour, and the reaction solution was concentrated under reduced pressure and separated by column chromatography (eluent: EA/PE=10%-30%) to obtain compound 20D (110 mg, 39%).

LC-MS (ESI): m/z=371.1 [M+H]⁺.

Step 4:

### 3-((2-methyl-2H-tetrazol-5-yl)(phenyl)methyl)-3,6-diazabicyclo[3.1.1]heptane (20E)

Compound 20E (75 mg, 93%) was obtained by using compound 20D (110 mg, 0.30 mmol) as a starting material with reference to the same operating method as that of compound 1e.

LC-MS (ESI): m/z=385.1 [M+H]⁺.

Step 5:

### N-(2-(2-(3-((2-methyl-2H-tetrazol-5-yl)(phenyl)methyl)-3,6-diazabicyclo[3.1.1]heptane-6-carbonyl)pyridin-4-yl)benzo[d]oxazol-5-yl)acetamide (Compound 20)

Intermediate 1 (49.0 mg, 0.17 mmol) was dissolved in N,N-dimethylformamide (1 mL), and the mixture was cooled to 0° C.; HATU (77.5 mg, 0.20 mmol) and DIPEA (70.0 mg, 0.54 mmol) were added; and then compound 20E (45.0 mg, 0.17 mmol) was added. After the addition, the mixture was stirred for 1 hour while the temperature was controlled at 0° C., and then ice water (5 mL) was added. The resulting solution was extracted twice with dichloromethane (20 mL×2). The organic phase was combined, dried over anhydrous sodium sulfate and filtered. The filtrate was concentrated under reduced pressure, and then the residue was separated by column chromatography (eluent: EA/DCM=30%-70%) to obtain compound 20 (35 mg, 38%).

¹H NMR (400 MHz, CD₃OD) δ 8.78-8.71 (m, 2H), 8.21-8.16 (m, 2H), 7.70-7.57 (m, 2H), 7.38-7.36 (m, 2H), 7.29-7.18 (m, 3H), 5.24 (d, 1H), 5.19-5.14 (m, 1H), 4.54 (s, 1H), 4.28 (s, 3H), 3.47-3.44 (m, 0.5H), 3.26-3.20 (m, 2H), 2.96-2.87 (m, 1.5H), 2.70-2.64 (m, 1H), 2.18 (s, 3H), 2.13-2.05 (m, 1H).

LC-MS (ESI): m/z=550.2 [M+H]⁺.

### Example 21

### N-(2-(2-(3-((2-methyl-2H-tetrazol-5-yl)(phenyl)methyl)-3,6-diazabicyclo[3.1.1]heptane-6-carbonyl)pyridin-4-yl)benzo[d]oxazol-5-yl)cyclopropanecarboxamide

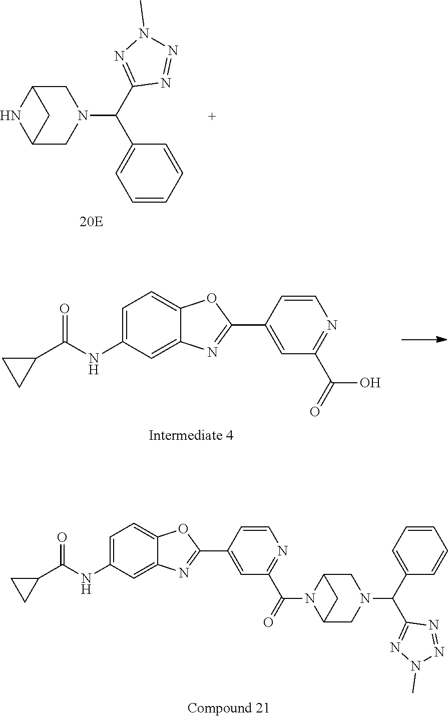 [SMILES: Cn1nnc(C(c2ccccc2)N2CC3CC(C2)N3)n1 ; O=C(O)c1cc(-c2nc3cc(NC(=O)C4CC4)ccc3o2)ccn1 ; Cn1nnc(C(c2ccccc2)N2CC3CC(C2)N3C(=O)c2cc(-c3nc4cc(NC(=O)C5CC5)ccc4o3)ccn2)n1]

Intermediate 4 (35.5 mg, 0.11 mmol) was dissolved in N,N-dimethylformamide (1 mL), and the mixture was cooled to 0° C.; HATU (45.6 mg, 0.12 mmol) and DIPEA (42.5 mg, 0.33 mmol) were added; and then compound 20E (30.0 mg, 0.11 mmol) was added. After the addition, the mixture was stirred for 1 hour while the temperature was controlled at 0° C., and then ice water (5 mL) was added. The resulting solution was extracted twice with dichloromethane (20 mL×2). The organic phase was combined, dried over anhydrous sodium sulfate and filtered. The filtrate was concentrated under reduced pressure, and then the residue was separated by column chromatography (eluent: EA/DCM=30%-70%) to obtain compound 21 (23 mg, 36%).

¹H NMR (400 MHz, CD₃OD) δ 8.79-8.70 (m, 2H), 8.22-8.17 (m, 2H), 7.71-7.58 (m, 2H), 7.38-7.36 (m, 2H), 7.29-7.18 (m, 3H), 5.24 (d, 1H), 5.19-5.16 (m, 1H), 4.54 (s, 1H), 4.28 (s, 3H), 3.47-3.44 (m, 0.5H), 3.25-3.11 (m, 2H), 2.95-2.87 (m, 1.5H), 2.70-2.64 (m, 1H), 2.11-2.07 (m, 1H), 1.84-1.77 (m, 1H), 1.0-0.87 (m, 4H).

LC-MS (ESI): m/z=576.2 [M+H]⁺.

### Example 22

### N-(2-(2-(3-((2-(2-fluoroethyl)-2H-tetrazol-5-yl)(phenyl)methyl)-3,8-diazabicyclo[3.2.1]octane-8-carbonyl)pyridin-4-yl)benzo[d]oxazol-5-yl)acetamide

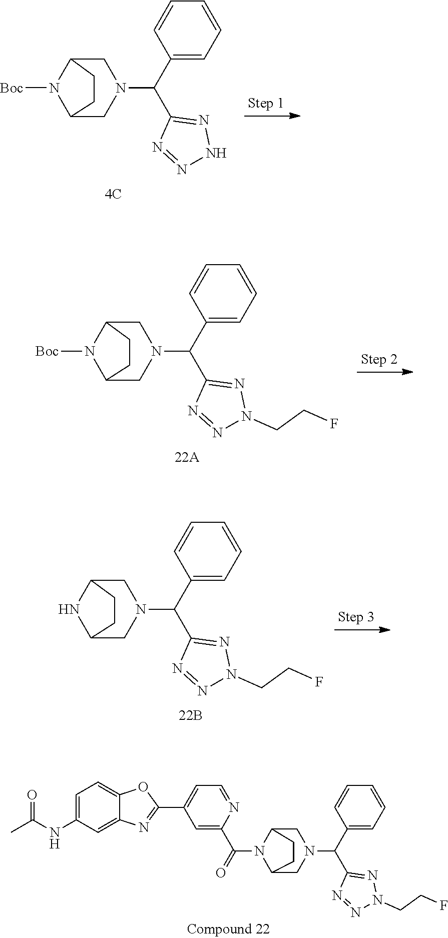 [SMILES: CC(C)(C)OC(=O)N1C2CCC1CN(C(c1ccccc1)c1nn[nH]n1)C2 ; CC(C)(C)OC(=O)N1C2CCC1CN(C(c1ccccc1)c1nnn(CCF)n1)C2 ; FCCn1nnc(C(c2ccccc2)N2CC3CCC(C2)N3)n1 ; CC(=O)Nc1ccc2oc(-c3ccnc(C(=O)N4C5CCC4CN(C(c4ccccc4)c4nnn(CCF)n4)C5)c3)nc2c1]

Compound 22

Step 1:

### tert-butyl-3-((2-(2-fluoroethyl)-2H-tetrazol-5-yl)(phenyl)methyl)-3,8-diazabicyclo[3.2.1]octane-8-carboxylate (22A)

N,N-dimethylformamide (4 mL), potassium carbonate (275 mg, 1.99 mmol) and 1-fluoro-2-iodoethane (692 mg, 4 mmol) were successively added to intermediate 4C (370 mg, 0.99 mmol). After the addition, the mixture was stirred at room temperature for 1 hour. Water (50 mL) was added, and the resulting solution was extracted twice with ethyl acetate (50 mL×2). The organic phase was combined, dried over anhydrous sodium sulfate and concentrated under reduced pressure, and then the residue was separated by column chromatography (PE:EA=1:1) to obtain 22A (330 mg, 79%).

¹H NMR (400 MHz, CDCl₃) δ 7.47-7.46 (d, 2H), 7.28-7.20 (m, 3H), 4.98 (s, 1H), 4.93-4.91 (t, 1H), 4.86-4.83 (t, 1H), 4.81-4.77 (m, 2H), 4.11-4.01 (m, 2H), 2.66-2.24 (m, 4H), 1.80-1.70 (m, 2H), 1.50 (s, 2H), 1.36 (s, 9H).

LC-MS (ESI): m/z=417.3 [M+H]⁺.

Step 2:

### 3-((2-(2-fluoroethyl)-2H-tetrazol-5-yl)(phenyl)methyl)-3,8-diazabicyclo[3.2.1]octane (22B)

Methanol (5 mL) and concentrated hydrochloric acid (2 mL) were successively added to compound 22A (330 mg, 0.79 mmol). The reaction solution was stirred at room temperature for 2 hours and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure to obtain compound 22B (130 mg, 52%).

LC-MS (ESI): m/z=317.3 [M+H]⁺.

Step 3:

### N-(2-(2-(3-((2-(2-fluoroethyl)-2H-tetrazol-5-yl)(phenyl)methyl)-3,8-diazabicyclo[3.2.1]octane-8-carbonyl)pyridin-4-yl)benzo[d]oxazol-5-yl)acetamide (Compound 22)

Intermediate 1 (55 mg, 0.18 mmol) was dissolved in N,N-dimethylformamide (2 mL), and the mixture was cooled to 0° C.; HATU (72 mg, 0.18 mmol) and DIPEA (62 mg, 0.48 mmol) were added; and then compound 22B (50 mg, 0.16 mmol) was added. After the addition, the mixture was stirred for 1 hour while the temperature was controlled at 0° C., and then ice water (10 mL) was added. The resulting solution was extracted twice with dichloromethane (50 mL×2). The organic phase was combined, dried over anhydrous sodium sulfate and concentrated under reduced pressure, and then the residue was separated by column chromatography (DCM:MeOH=20:1) to obtain compound 22 (50 mg, 49%).

¹H NMR (400 MHz, CDCl₃) δ 8.78-8.71 (m, 1H), 8.56-8.54 (d, 1H), 8.17-8.15 (m, 1H), 8.04 (s, 1H), 7.68-7.56 (m, 4H), 7.44-7.38 (m, 3H), 5.56 (d, 1H), 4.94-4.90 (m, 4H), 3.39-3.37 (m, 2H), 3.19-2.90 (m, 4H), 2.19 (s, 3H), 2.18-2.09 (m, 4H).

LC-MS (ESI): m/z=596.3 [M+H]⁺.

### Example 23

### N-(2-(2-(6-((2-methyl-2H-tetrazol-5-yl)(phenyl)methyl)-3,6-diazabicyclo[3.1.1]heptane-3-carbonyl)pyridin-4-yl)benzo[d]oxazol-5-yl)acetamide

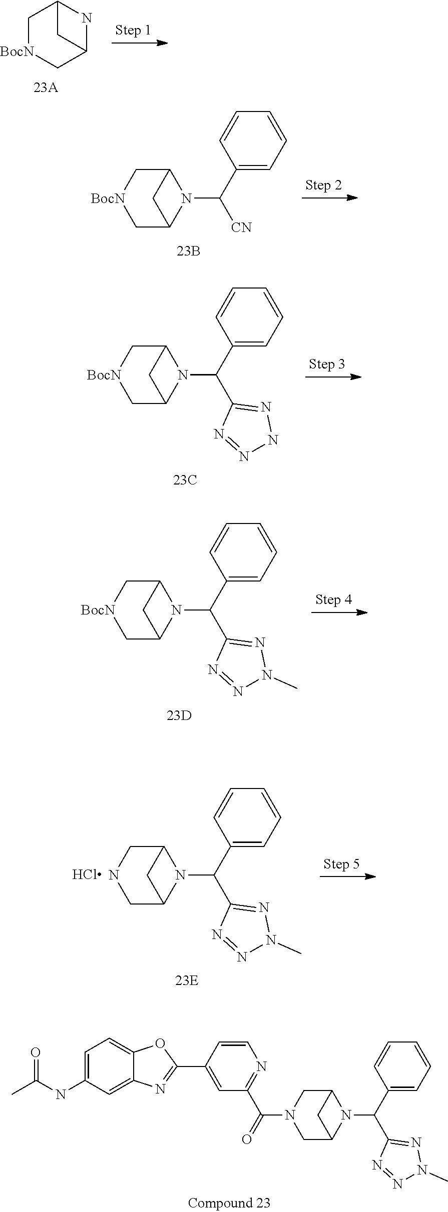 [SMILES: CC(C)(C)OC(=O)N1CC2CC(C1)N2 ; CC(C)(C)OC(=O)N1CC2CC(C1)N2C(C#N)c1ccccc1 ; CC(C)(C)OC(=O)N1CC2CC(C1)N2C(c1ccccc1)c1nn[nH]n1 ; Cn1nnc(C(c2ccccc2)N2C3CC2CN(C(=O)OC(C)(C)C)C3)n1 ; Cn1nnc(C(c2ccccc2)N2C3CNCC2C3)n1 ; CC(=O)Nc1ccc2oc(-c3ccnc(C(=O)N4CC5CC(C4)N5C(c4ccccc4)c4nnn(C)n4)c3)nc2c1]

Step 1:

### tert-butyl 6-(cyano(phenyl)methyl)-3,6-diazabicyclo[3.1.1]heptane-3-carboxylate (23B)

Compound 23A (2.0 g, 10.09 mmol) was dissolved in acetonitrile (50 mL). Benzaldehyde (1.1 g, 10.58 mmol), TMSCN (1.2 g, 12.10 mmol) and iodine (256 mg, 1.01 mmol) were added at room temperature. The reaction solution was stirred at room temperature for 12 hours, and water (100 mL) was added. The residue was extracted with ethyl acetate (100 mL×2). The combined organic phase was dried over anhydrous sodium sulfate and concentrated under reduced pressure, and then the residue was separated and purified by silica gel column chromatography (PE:EA=2:1) to obtain 23B (1.7 g, 54%).

LC-MS (ESI): m/z=314.2 [M+H]⁺.

Step 2:

### tert-butyl 6-(phenyl(2H-tetrazol-5-yl)methyl)-3,6-diazabicyclo[3.1.1]heptane-3-carboxylate (23C)

Compound 23B (500 mg, 1.60 mmol) was dissolved in isopropanol (10 mL). Sodium azide (208 mg, 3.20 mmol) and zinc bromide (180 mg, 0.80 mmol) were added at room temperature. The reaction solution was stirred at 90° C. for 12 hours, cooled, adjusted to pH 5-6 or so with 1 M hydrochloric acid and concentrated under reduced pressure, and the residue was separated and purified by silica gel column chromatography (DCM:MeOH=20:1) to obtain 23C (400 mg, 70%).

LC-MS (ESI): m/z=357.2 [M+H]⁺.

Step 3:

### tert-butyl 6-((2-methyl-2H-tetrazol-5-yl)(phenyl)methyl)-3,6-diazabicyclo[3.1.1]heptane-3-carboxylate (23D)

Compound 23D (120 mg, 29%) was obtained by using compound 23C (400 mg, 1.12 mmol) as a starting material with reference to the same step as that of compound 1.

LC-MS (ESI): m/z=371.2 [M+H]⁺.

Step 4:

### 6-((2-methyl-2H-tetrazol-5-yl)(phenyl)methyl)-3,6-diazabicyclo[3.1.1]heptanes (23E)

Compound 23E (100 mg, 100%) was obtained by using compound 23D (120 mg, 0.32 mmol) as a starting material with reference to the same step as that of compound 1.

LC-MS (ESI): m/z=271.2 [M+H]⁺.

Step 5:

### N-(2-(2-(6-((2-methyl-2H-tetrazol-5-yl)(phenyl)methyl)-3,6-diazabicyclo[3.1.1]heptane-3-carbonyl)pyridin-4-yl)benzo[d]oxazol-5-yl)acetamide (Compound 23)

Compound 23 (40 mg, 23%) was obtained by using compound 23E (100 mg, 1.12 mmol) as a starting material with reference to the same step as that of compound 1.

¹H NMR (400 MHz, CD₃OD) δ 8.85 (d, 1H), 8.47 (d, 1H), 8.21-8.25 (m, 1H), 8.20 (d, 1H), 7.66-7.70 (m, 3H), 7.56-7.59 (m, 1H), 7.36-7.40 (m, 1H), 7.24-7.34 (m, 2H), 5.41 (d, 1H), 4.33 (s, 1.5H), 4.26 (s, 1.5H), 4.01-4.16 (m, 2H), 3.84-3.87 (m, 1H), 3.61-3.77 (m, 2H), 3.43-3.54 (m, 1H), 2.68-2.72 (m, 1H), 2.17 (s, 3H), 1.68 (t, 1H).

LC-MS (ESI): m/z=550.2 [M+H]⁺.

### Example 24

### (1S)-2,2-difluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide

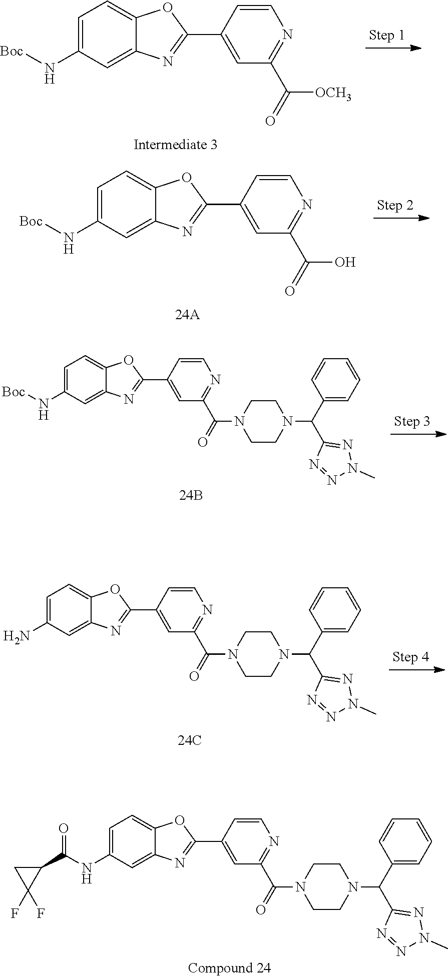 [SMILES: COC(=O)c1cc(-c2nc3cc(NC(=O)OC(C)(C)C)ccc3o2)ccn1 ; CC(C)(C)OC(=O)Nc1ccc2oc(-c3ccnc(C(=O)O)c3)nc2c1 ; Cn1nnc(C(c2ccccc2)N2CCN(C(=O)c3cc(-c4nc5cc(NC(=O)OC(C)(C)C)ccc5o4)ccn3)CC2)n1 ; Cn1nnc(C(c2ccccc2)N2CCN(C(=O)c3cc(-c4nc5cc(N)ccc5o4)ccn3)CC2)n1 ; Cn1nnc(C(c2ccccc2)N2CCN(C(=O)c3cc(-c4nc5cc(NC(=O)[C@@H]6CC6(F)F)ccc5o4)ccn3)CC2)n1]

Step 1:

### 4-(5-((tert-butoxycarbonyl)amino)benzo[d]oxazol-2-yl)picolinic Acid (24A)

Anhydrous methanol (10 mL) and NaOH (0.16 g, 4.0 mmol, 2 mL) aqueous solution were successively added to compound intermediate 3 (0.3 g, 0.81 mmol). The mixture was stirred at room temperature for 10 h, concentrated under reduced pressure to remove most of methanol, adjusted to pH=2-3 by dropwise adding 1 N hydrochloric acid and filtered. The filter cake was washed with water (3 mL) and dried to obtain intermediate 24A (180 mg, 63%).

LC-MS (ESI): m/z=356.1 [M+H]⁺.

Step 2:

### tert-butyl (2-(2-(4-((2-methyl-2H-tetrazol-5-yl)(phenyl)methyl)piperazine-1-carbonyl)pyridin-4-yl)benzo[d]oxazol-5-yl)carbamate (24B)

At room temperature, DMF (5 mL), intermediate 2 (89 mg, 0.3 mmol), HATU (0.13 g, 0.33 mmol) and DIPEA (0.12 g, 0.9 mmol) were successively added to compound 24A (107 mg, 0.3 mmol). The reaction was stirred for 1 hour. The reaction solution was poured into water (50 mL) and extracted with ethyl acetate (20 mL×2). The organic phase was concentrated, and the residue was separated and purified by silica gel column chromatography (PE:EA=1:3) to obtain compound 24B (90 mg, 50%).

LC-MS (ESI): m/z=596.3 [M+H]⁺.

Step 3:

### (4-(5-aminobenzo[d]oxazol-2-yl)pyridin-2-yl)(4-((2-methyl-2H-tetrazol-5-yl)(phenyl)methyl)piperazin-1-yl)methanone (24C)

Dichloromethane (1 mL) and trifluoroacetic acid (1 mL) were successively added to compound 24B (90 mg, 0.15 mmol). The reaction solution was stirred at room temperature for 2 h and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure, and then the residue was separated and purified by silica gel column chromatography (DCM:MeOH=20:1) to obtain intermediate 24C (70 mg, 95%).

LC-MS (ESI): m/z=496.2 [M+H]⁺.

Step 4:

### (1S)-2,2-difluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide (Compound 24)

At room temperature, DMF (5 mL), (S)-2,2-difluorocyclopropane-1-carboxylic acid (19 mg, 0.154 mmol), HATU (69 mg, 0.18 mmol) and DIPEA (0.05 g, 0.42 mmol) were successively added to compound 24C (70 mg, 0.14 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 24 (20 mg, 24%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate);

gradient elution: the content of mobile phase A rises from 40% to 70%; flow rate: 15 ml/min; elution time: 18 min; retention time: 11.85 min.

¹H NMR (400 MHz, DMSO-d₆) δ 10.66 (s, 1H), 8.82 (d, 1H), 8.22-8.23 (m, 2H), 8.16-8.17 (m, 1H), 7.82-7.84 (m, 1H), 7.58-7.61 (m, 3H), 7.36-7.45 (m, 3H), 5.57 (s, 1H), 4.38 (s, 3H), 3.63-3.79 (m, 4H), 2.67-2.88 (m, 5H), 1.95-2.09 (m, 2H).

LC-MS (ESI): m/z=600.3 [M+H]⁺.

### Example 25

### (1S,2R)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide

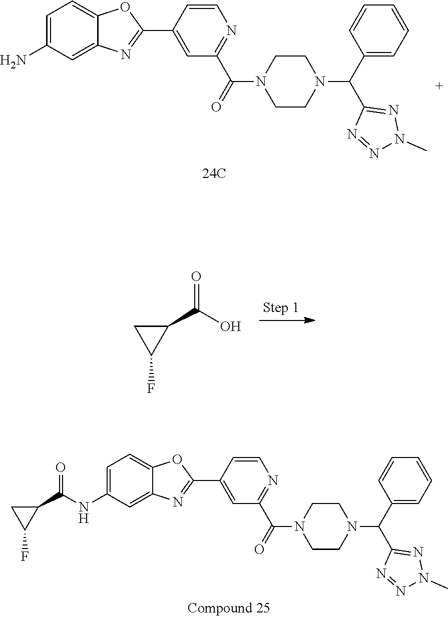 [SMILES: Cn1nnc(C(c2ccccc2)N2CCN(C(=O)c3cc(-c4nc5cc(N)ccc5o4)ccn3)CC2)n1 ; O=C(O)[C@@H]1C[C@H]1F ; Cn1nnc(C(c2ccccc2)N2CCN(C(=O)c3cc(-c4nc5cc(NC(=O)[C@@H]6C[C@H]6F)ccc5o4)ccn3)CC2)n1]

Step 1:

### (1S,2R)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide (Compound 25)

Compound 25 (50 mg, 43%) was obtained by using compound 24C (100 mg, 0.20 mmol) as a starting material with reference to the same step as that of compound 24.

¹H NMR (400 MHz, DMSO-d₆) δ 10.60 (s, 1H), 8.81 (d, 1H), 8.19-8.20 (m, 2H), 8.14-8.15 (m, 1H), 7.80-7.82 (m, 1H), 7.53-7.59 (m, 3H), 7.33-7.42 (m, 3H), 5.37 (s, 1H), 4.83-5.00 (m, 1H), 4.37 (s, 3H), 3.74-3.75 (m, 2H), 3.55-3.56 (m, 2H), 2.63-2.73 (m, 4H), 2.29-2.38 (m, 1H), 1.51-1.57 (m, 1H), 1.25-1.30 (m, 1H).

LC-MS (ESI): m/z=582.3 [M+H]⁺.

### Example 26

### (1S,2S)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide

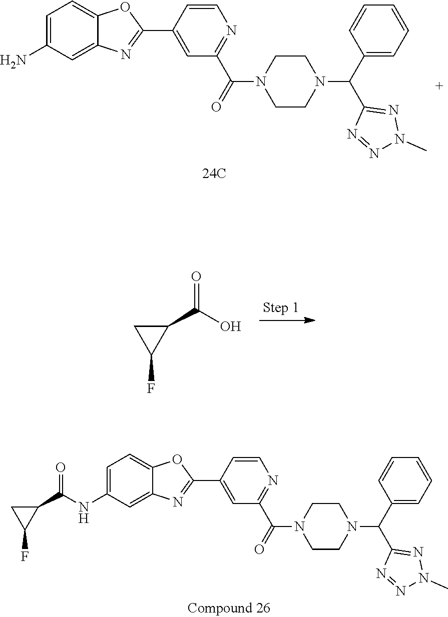 [SMILES: Cn1nnc(C(c2ccccc2)N2CCN(C(=O)c3cc(-c4nc5cc(N)ccc5o4)ccn3)CC2)n1 ; O=C(O)[C@@H]1C[C@@H]1F ; Cn1nnc(C(c2ccccc2)N2CCN(C(=O)c3cc(-c4nc5cc(NC(=O)[C@@H]6C[C@@H]6F)ccc5o4)ccn3)CC2)n1]

Step 1:

### (1S,2S)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide (Compound 26)

Compound 26 (48 mg, 41%) was obtained by using compound 24C (100 mg, 0.20 mmol) as a starting material with reference to the same step as that of compound 24.

¹H NMR (400 MHz, DMSO-d₆) δ 10.48 (s, 1H), 8.81 (d, 1H), 8.19-8.24 (m, 2H), 8.14-8.15 (m, 1H), 7.79-7.81 (m, 1H), 7.60-7.63 (m, 1H), 7.51-7.53 (m, 2H), 7.31-7.40 (m, 3H), 5.26 (s, 1H), 4.85-5.05 (m, 1H), 4.36 (s, 3H), 3.73-3.74 (m, 2H), 3.52-3.53 (m, 2H), 2.67-2.68 (m, 1H), 2.49-2.51 (m, 3H), 2.01-2.08 (m, 1H), 1.62-1.72 (m, 1H), 1.14-1.21 (m, 1H).

LC-MS (ESI): m/z=582.3 [M+H]⁺.

### Example 27

### (1R,2S)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide

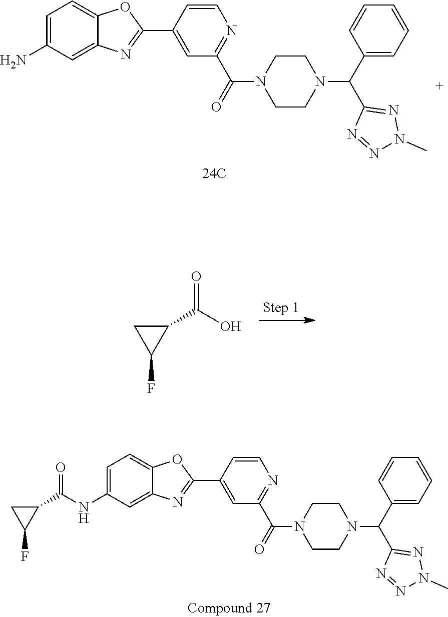 [SMILES: Cn1nnc(C(c2ccccc2)N2CCN(C(=O)c3cc(-c4nc5cc(N)ccc5o4)ccn3)CC2)n1 ; O=C(O)[C@H]1C[C@@H]1F ; Cn1nnc(C(c2ccccc2)N2CCN(C(=O)c3cc(-c4nc5cc(NC(=O)[C@H]6C[C@@H]6F)ccc5o4)ccn3)CC2)n1]

Step 1:

### (1R,2S)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide (Compound 27)

Compound 27 (8 mg, 16%) was obtained by using compound 24C (100 mg, 0.20 mmol) as a starting material with reference to the same step as that of compound 24.

¹H NMR (400 MHz, CDCl₃) δ 8.73 (d, 1H), 8.40 (s, 1H), 8.13 (d, 1H), 7.98 (s, 1H), 7.79 (d, 1H), 7.67 (dd, 2H), 7.52 (d, 2H), 7.46 (dd, 2H), 4.90 (dd, 1H), 4.05 (d, 4H), 3.25 (s, 4H), 3.00 (s, 3H), 2.05 (dd, 2H), 1.66-1.38 (m, 2H).

LC-MS (ESI): m/z=582.3 [M+H]⁺.

### Example 28

### (1R,2R)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide

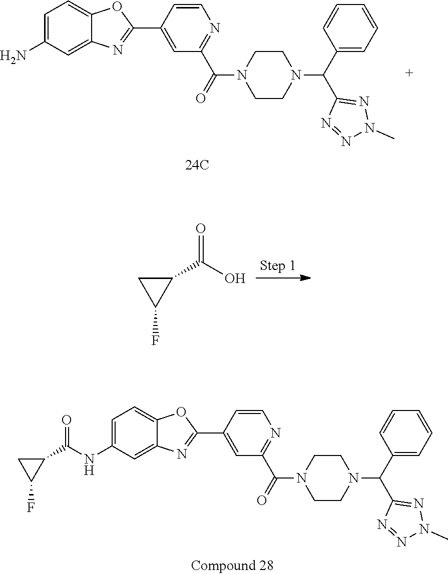 [SMILES: Cn1nnc(C(c2ccccc2)N2CCN(C(=O)c3cc(-c4nc5cc(N)ccc5o4)ccn3)CC2)n1 ; O=C(O)[C@H]1C[C@H]1F ; Cn1nnc(C(c2ccccc2)N2CCN(C(=O)c3cc(-c4nc5cc(NC(=O)[C@H]6C[C@H]6F)ccc5o4)ccn3)CC2)n1]

Step 1:

### (1R,2R)-2-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide (Compound 28)

Compound 28 (10 mg, 17%) was obtained by using compound 24C (100 mg, 0.20 mmol) as a starting material with reference to the same step as that of compound 24.

¹H NMR (400 MHz, CDCl₃) δ 8.73 (d, 1H), 8.40 (s, 1H), 8.13 (d, 1H), 7.98 (s, 1H), 7.79 (d, 1H), 7.67 (dd, 2H), 7.52 (d, 2H), 7.46 (dd, 2H), 4.90 (dd, 1H), 4.05 (d, 4H), 3.00 (s, 3H), 2.77 (s, 4H), 2.05 (dd, 2H), 1.66-1.38 (m, 2H).

LC-MS (ESI): m/z=582.3 [M+H]⁺.

### Example 29

### (1S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-2,2-difluorocyclopropane-1-carboxamide

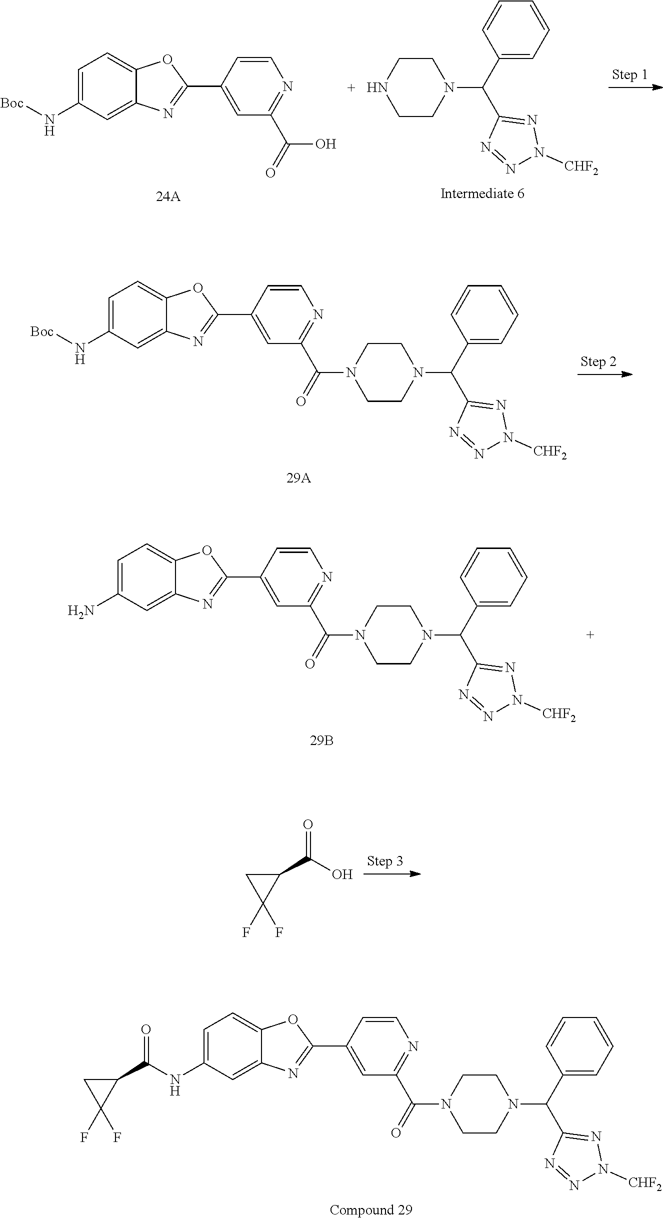

Step 1:

### tert-butyl (2-(2-(4-((2-(difluoromethyl)-2H-tetrazol-5-yl)(phenyl)methyl)piperazine-1-carbonyl)pyridin-4-yl)benzo[d]oxazol-5-yl)carbamate (29A)

At room temperature, DMF (5 mL), intermediate 6 (88 mg, 0.3 mmol), HATU (0.13 g, 0.33 mmol) and DIPEA (0.12 g, 0.9 mmol) were successively added to compound 24A (107 mg, 0.3 mmol). The reaction was stirred for 1 hour. The reaction solution was poured into water (50 mL) and extracted with ethyl acetate (20 mL×2). The organic phase was concentrated, and the residue was separated and purified by silica gel column chromatography (PE:EA=1:3) to obtain compound 29A (95 mg, 50%).

LC-MS (ESI): m/z=632.2 [M+H]⁺.

Step 2:

### (4-(5-aminobenzo[d]oxazol-2-yl)pyridin-2-yl)(4-((2-(difluoromethyl)-2H-tetrazol-5-yl)(phenyl)methyl)piperazin-1-yl)methanone (29B)

Dichloromethane (1 mL) and trifluoroacetic acid (1 mL) were successively added to compound 29A (95 mg, 0.15 mmol). The reaction solution was stirred at room temperature for 2 h and adjusted to pH=8-9 by dropwise adding saturated aqueous sodium carbonate solution. The resulting reaction solution was extracted with dichloromethane (50 mL×2). The combined organic phase was dried over anhydrous sodium sulfate and concentrated under reduced pressure, and then the residue was separated and purified by silica gel column chromatography (DCM:MeOH=20:1) to obtain intermediate 29B (75 mg, 94%).

LC-MS (ESI): m/z=532.2 [M+H]⁺.

Step 4:

### (1S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-2,2-difluorocyclopropane-1-carboxamide (Compound 29)

At room temperature, DMF (5 mL), (S)-2,2-difluorocyclopropane-1-carboxylic acid (19 mg, 0.154 mmol), HATU (69 mg, 0.18 mmol) and DIPEA (50 mg, 0.42 mmol) were successively added to compound 29B (75 mg, 0.14 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 29 (25 mg, 28%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.07 min.

¹H NMR (400 MHz, CD₃OD) δ 8.79-8.80 (m, 1H), 8.33-8.34 (m, 1H), 8.20-8.22 (m, 2H), 8.19 (t, 1H), 7.69-7.71 (m, 1H), 7.57-7.61 (m, 3H), 7.35-7.43 (m, 3H), 5.41 (s, 1H), 3.91-3.92 (m, 2H), 3.61-3.62 (m, 2H), 2.66-2.85 (m, 5H), 2.08-2.17 (m, 1H), 1.81-1.90 (m, 1H).

LC-MS (ESI): m/z=636.2 [M+H]⁺.

### Example 30

### (1S,2R)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-2-fluorocyclopropane-1-carboxamide

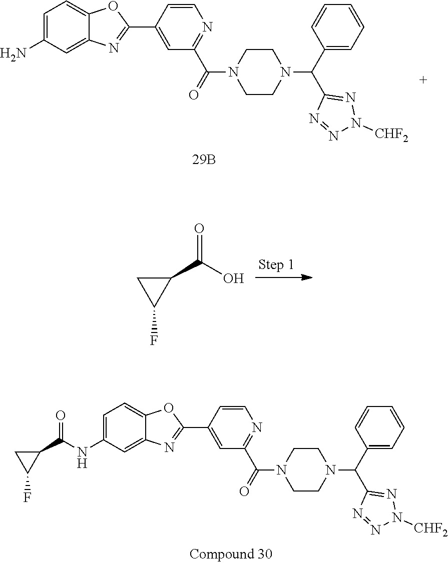 [SMILES: Nc1ccc2oc(-c3ccnc(C(=O)N4CCN(C(c5ccccc5)c5nnn(C(F)F)n5)CC4)c3)nc2c1 ; "+" ; O=C(O)[C@@H]1C[C@H]1F ; O=C(Nc1ccc2oc(-c3ccnc(C(=O)N4CCN(C(c5ccccc5)c5nnn(C(F)F)n5)CC4)c3)nc2c1)[C@@H]1C[C@H]1F]

Step 1:

### (1S,2R)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide (Compound 30)

Compound 30 (50 mg, 41%) was obtained by using compound 29B (100 mg, 0.20 mmol) as a starting material with reference to the same step as that of compound 29.

¹H NMR (400 MHz, DMSO-d₆) δ 10.59 (s, 1H), 8.79-8.80 (m, 1H), 8.55 (t, 1H), 8.17-8.20 (m, 2H), 8.12-8.14 (m, 1H), 7.79-7.81 (m, 1H), 7.56-7.59 (m, 1H), 7.48-7.50 (m, 2H), 7.31-7.41 (m, 3H), 5.31 (s, 1H), 4.84-5.00 (m, 1H), 3.70-3.72 (m, 2H), 3.49-3.50 (m, 2H), 2.58-2.60 (m, 1H), 2.46-2.48 (m, 2H), 2.29-2.37 (m, 2H), 1.50-1.59 (m, 1H), 1.24-1.32 (m, 1H).

LC-MS (ESI): m/z=618.3 [M+H]⁺.

### Example 31

### (1S,2S)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide

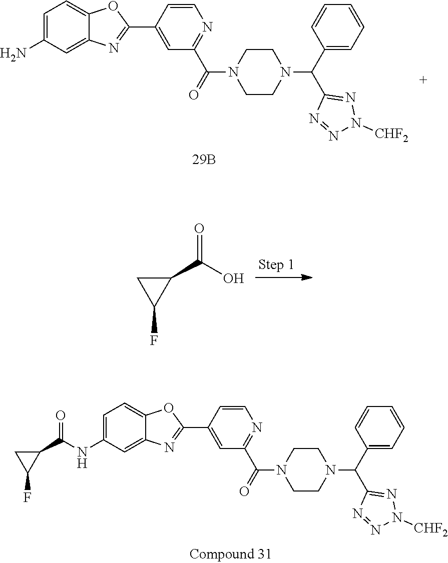 [SMILES: Nc1ccc2oc(-c3ccnc(C(=O)N4CCN(C(c5ccccc5)c5nnn(C(F)F)n5)CC4)c3)nc2c1 ; "+" ; O=C(O)[C@@H]1C[C@@H]1F ; O=C(Nc1ccc2oc(-c3ccnc(C(=O)N4CCN(C(c5ccccc5)c5nnn(C(F)F)n5)CC4)c3)nc2c1)[C@@H]1C[C@@H]1F]

Step 1:

### (1S,2S)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide (Compound 31)

Compound 31 (48 mg, 39%) was obtained by using compound 29B (100 mg, 0.20 mmol) as a starting material with reference to the same step as that of compound 29.

¹H NMR (400 MHz, DMSO-d₆) δ 10.48 (s, 1H), 8.79-8.81 (m, 1H), 8.56 (t, 1H), 8.18-8.24 (m, 2H), 8.13-8.15 (m, 1H), 7.78-7.81 (m, 1H), 7.61-7.63 (m, 1H), 7.50-7.52 (m, 2H), 7.32-7.42 (m, 3H), 5.38 (s, 1H), 4.85-5.06 (m, 1H), 3.71-3.73 (m, 2H), 3.49-3.51 (m, 2H), 2.59-2.63 (m, 1H), 2.49-2.52 (m, 2H), 2.41-2.43 (m, 1H), 2.01-2.08 (m, 1H), 1.62-1.72 (m, 1H), 1.13-1.21 (m, 1H).

LC-MS (ESI): m/z=618.3 [M+H]⁺.

### Example 32

### (1R,2S)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide

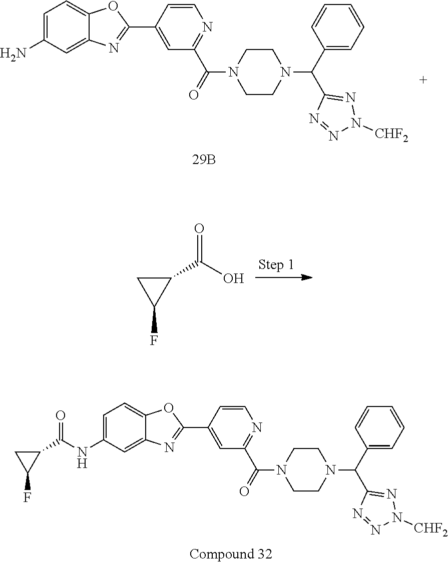 [SMILES: Nc1ccc2oc(-c3ccnc(C(=O)N4CCN(C(c5ccccc5)c5nnn(C(F)F)n5)CC4)c3)nc2c1 ; "+" ; O=C(O)[C@H]1C[C@@H]1F ; O=C(Nc1ccc2oc(-c3ccnc(C(=O)N4CCN(C(c5ccccc5)c5nnn(C(F)F)n5)CC4)c3)nc2c1)[C@H]1C[C@@H]1F]

Step 1:

### (1R,2S)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide (Compound 32)

Compound 32 (25 mg, 82%) was obtained by using compound 29B (50 mg, 0.20 mmol) as a starting material with reference to the same step as that of compound 29.

¹H NMR (400 MHz, CDCl₃) δ 8.70 (d, 1H), 8.48 (s, 1H), 8.15 (dd, 1H), 8.02 (s, 1H), 7.97-7.90 (m, 1H), 7.70 (s, 1H), 7.55 (s, 1H), 7.49 (dd, 4H), 4.99 (t, 1H), 4.45 (s, 2H), 3.58 (s, 1H), 2.20-1.96 (m, 1H), 1.81-1.35 (m, 7H).

LC-MS (ESI): m/z=618.2 [M+H]⁺.

### Example 33

### (1R,2R)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide

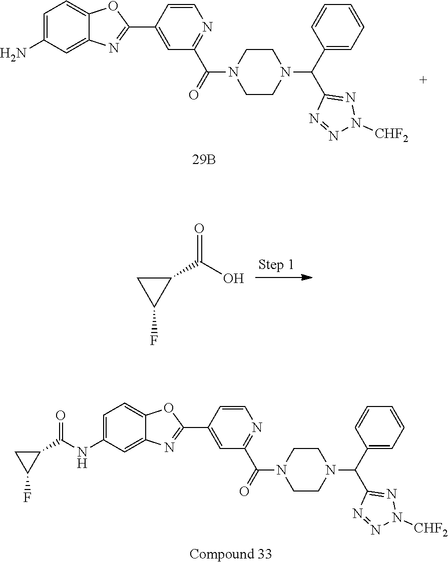 [SMILES: Nc1ccc2oc(-c3ccnc(C(=O)N4CCN(C(c5ccccc5)c5nnn(C(F)F)n5)CC4)c3)nc2c1 ; "+" ; O=C(O)[C@H]1C[C@H]1F ; O=C(Nc1ccc2oc(-c3ccnc(C(=O)N4CCN(C(c5ccccc5)c5nnn(C(F)F)n5)CC4)c3)nc2c1)[C@H]1C[C@H]1F]

Step 1:

### (1R,2R)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide (Compound 33)

Compound 33 (15 mg, 38%) was obtained by using compound 29B (50 mg, 0.10 mmol) as a starting material with reference to the same step as that of compound 29.

¹H NMR (400 MHz, CDCl₃) δ 8.70 (d, 1H), 8.48 (s, 1H), 8.15 (dd, 1H), 8.02 (s, 1H), 7.97-7.90 (m, 1H), 7.70 (s, 1H), 7.55 (s, 1H), 7.49 (dd, 4H), 4.99 (t, 1H), 4.45 (s, 2H), 3.58 (s, 1H), 2.20-1.96 (m, 1H), 1.81-1.35 (m, 7H).

LC-MS (ESI): m/z=618.2 [M+H]⁺.

### Example 34

### (1R,2R)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide

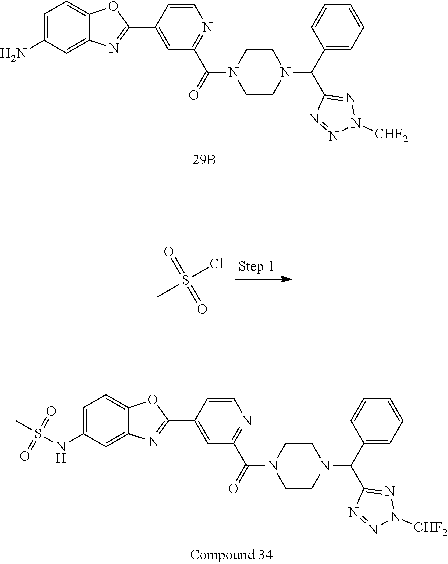

Step 1:

### (1R,2R)-2-fluoro-N-(2-(2-(4-((2-difluoromethyl-2H-tetrazol-5-yl)(phenyl)methyl)piperazine-1-carbonyl)pyridin-4-yl)benzo[d]oxazol-5-yl)cyclopropane-1-carboxamide (Compound 34)

At room temperature, DCM (15 mL), triethylamine (58 mg, 0.57 mmol) and methanesulfonyl chloride (43 mg, 0.38 mmol) were successively added to compound 29B (100 mg, 0.19 mmol). The reaction was stirred for 3 hours. The reaction solution was poured into water (50 mL) and extracted with dichloromethane (50 mL×2). The organic phase was concentrated, and the residue was separated and purified by HPLC to obtain compound 34 (25 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.96 min

¹H NMR (400 MHz, CD₃OD) δ 8.79 (d, 1H), 8.30 (t, 1H), 8.33-8.34 (m, 1H), 8.21-8.23 (m, 1H), 7.71-7.74 (m, 2H), 7.42-7.50 (m, 5H), 7.39-7.41 (m, 1H), 5.58 (s, 1H), 3.90-3.92 (m, 2H), 3.67-3.68 (m, 2H), 2.98 (s, 3H), 2.79-2.80 (m, 2H), 2.69-2.70 (m, 2H).

LC-MS (ESI): m/z=610.2 [M+H]⁺.

### Example 35

### N-(2-(2-(4-((2-methyl-2H-tetrazol-5-yl)(phenyl)methyl)piperazine-1-carbonyl)pyridin-4-yl)benzo[d]oxazol-5-yl)cyclopropanecarboxamide

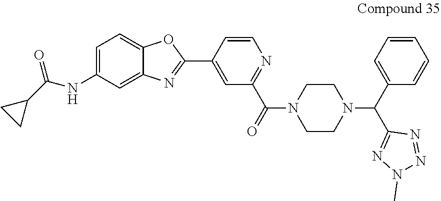 [SMILES: Cn1nnc(C(c2ccccc2)N2CCN(C(=O)c3cc(-c4nc5cc(NC(=O)C6CC6)ccc5o4)ccn3)CC2)n1]

Compound 35 was obtained by using intermediate 4 and intermediate 2 according to the same synthetic method of Example 2.

¹H NMR (400 MHz, CD₃OD) δ 8.77 (d, 1H), 8.28 (s, 1H), 8.23-8.10 (m, 2H), 7.58 (m, 4H), 7.41-7.21 (m, 3H), 5.01 (s, 1H), 4.54 (s, 4H), 4.35 (s, 3H), 3.84 (s, 2H), 3.56 (t, 2H), 1.89-1.72 (m, 1H), 1.05-0.79 (m, 4H).

LC-MS (ESI): m/z=564.3 [M+H]⁺.

### Example 36

### N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclopropanecarboxamide

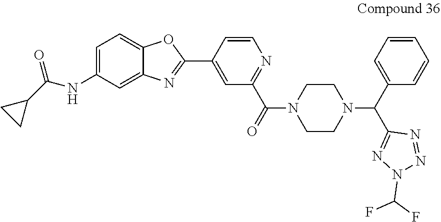 [SMILES: O=C(Nc1ccc2oc(-c3ccnc(C(=O)N4CCN(C(c5ccccc5)c5nnn(C(F)F)n5)CC4)c3)nc2c1)C1CC1]

Compound 36 was obtained by using intermediate 4 and intermediate 6 according to the same synthetic method of Example 2.

¹H NMR (400 MHz, CDCl₃) δ 8.71 (d, 1H), 8.34 (s, 1H), 8.07 (m, 1H), 7.97 (s, 1H), 7.77 (s, 1H), 7.52 (m, 4H), 7.35 (m, 3H), 5.19 (s, 1H), 3.93 (s, 2H), 3.77 (d, 2H), 2.64 (m, 4H), 1.60-1.49 (m, 1H), 1.12 (m, 2H), 0.87 (td, 2H).

LC-MS (ESI): m/z=600.2 [M+H]⁺.

### Example 37

### 1-(2-(2-(4-((2-(difluoromethyl)-2H-tetrazol-5-yl)(phenyl)methyl)piperazine-1-carbonyl)pyridin-4-yl)benzo[d]oxazol-5-yl)pyrrolidin-2-one

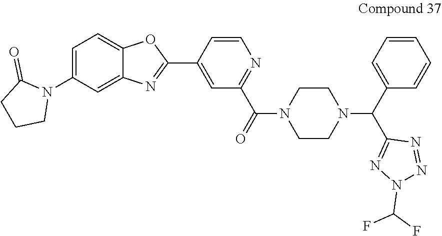 [SMILES: O=C(c1cc(-c2nc3cc(N4CCCC4=O)ccc3o2)ccn1)N1CCN(C(c2ccccc2)c2nnn(C(F)F)n2)CC1]

Compound 37 was obtained by using intermediate 5 and intermediate 6 according to the same synthetic method of Example 2.

¹H NMR (400 MHz, CDCl₃) δ 8.73 (d, 1H), 8.39 (s, 1H), 8.10 (m, 1H), 7.89 (m, 2H), 7.76-7.47 (t, 1H), 7.61 (m, 1H), 7.60-7.52 (m, 2H), 7.47-7.28 (m, 3H), 5.11 (s, 1H), 3.95 (t, 2H), 3.88 (t, 2H), 3.67 (t, 2H), 2.69 (m, 3H), 2.61 (m, 1H), 2.57-2.49 (m, 1H), 2.42 (m, 1H), 2.27-2.18 (m, 2H).

LC-MS (ESI): m/z=600.3 [M+H]⁺.

### Example 38

### (4-((2-methyl-2H-tetrazol-5-yl)(phenyl)methyl)piperazin-1-yl)(4-(5-(((S)-tetrahydrofuran-3-yl)oxy)benzo[d]oxazol-2-yl)pyridin-2-yl)methanone

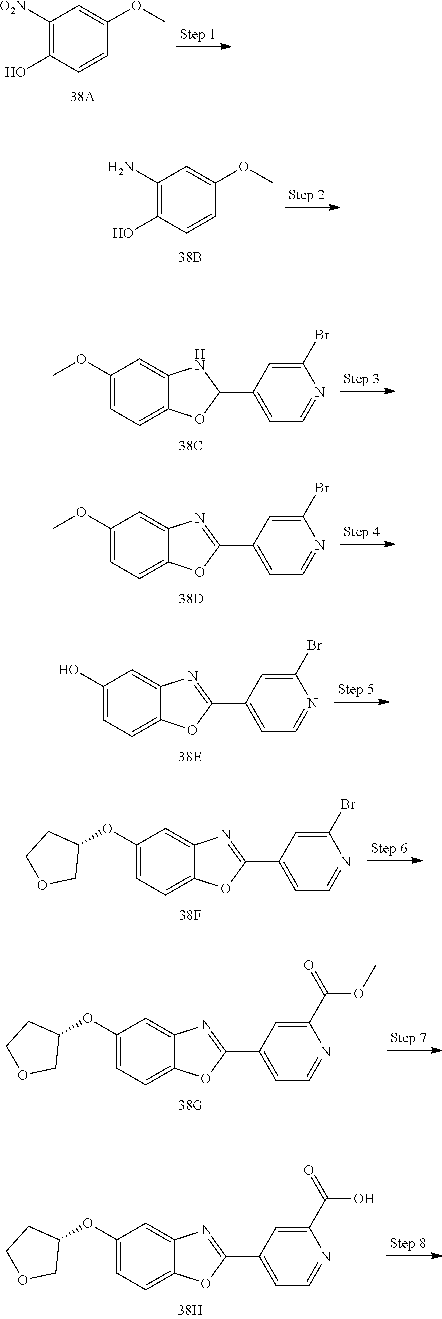

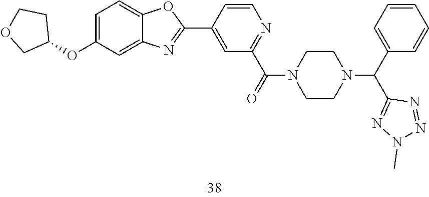 [SMILES: Cn1nnc(C(c2ccccc2)N2CCN(C(=O)c3cc(-c4nc5cc(O[C@H]6CCOC6)ccc5o4)ccn3)CC2)n1]

Step 1:

### 2-amino-4-methoxyphenol (38B)

Compound 38A (22 g, 0.13 mol) and 10% palladium on carbon (2 g) were added to methanol (150 mL), and the mixture was reacted at room temperature for 16 h under hydrogen replacement. The reaction solution was filtered, and the filtrate was concentrated under reduced pressure to obtain 38B (17 g, 94%), which was used directly in the next step without purification.

Step 2:

### 2-(2-bromopyridin-4-yl)-5-methoxy-2,3-dihydrobenzo[d]oxazole (38C)

Compound 38B (16.7 g, 0.12 mol) and 2-bromopyridine-4-carbaldehyde (22.3 g, 0.12 mol) were added to methanol (150 mL), and the mixture was reacted at room temperature for 1 h and directly concentrated under reduced pressure to obtain a crude product of 38C.

Step 3:

### 2-(2-bromopyridin-4-yl)-5-methoxybenzo[d]oxazole (38D)

Compound 38C from the previous step was added to dichloromethane (200 ml), and then 58% activated manganese dioxide (36 g, 0.24 mol) was added. The mixture was reacted at room temperature for 1 h. The reaction solution was directly filtered. The filtrate was concentrated under reduced pressure, and then the residue was slurried with methanol and filtered. The filtered solid was washed with methanol and then dried to obtain 38D (27.5 g, 75%).

LC-MS (ESI): m/z=305,307 [M+H]⁺.

Step 4:

### 2-(2-bromopyridin-4-yl)benzo[d]oxazol-5-ol (38E)

Compound 38D (4.1 g, 13.44 mmol) was added to dichloromethane (40 mL), and boron tribromide (78 mmol, 78 ml) was added dropwise at 0° C. The reaction solution was stirred at room temperature for 2 hours. The reaction was quenched with methanol and then adjusted to pH=7-8 with aqueous sodium bicarbonate solution. The solid undissolved in the system was filtered directly, and the filtered solid was washed with water and dried to obtain compound 38E (3.4 g, 87%).

LC-MS (ESI): m/z=291,293 [M+H]⁺.

Step 5:

### (S)-2-(2-bromopyridin-4-yl)-5-((tetrahydrofuran-3-yl)oxy)benzo[d]oxazole (38F)

Compound 38E (3.3 g, 11.3 mmol), (R)-tetrahydrofuran-3-yl methanesulfonate (2.7 g, 15.9 mmol) and cesium carbonate (9.2 g, 28.25 mmol) were added to N,N-dimethylformamide (30 mL), and the mixture was reacted at 90° C. for 2 hours. The reaction solution was cooled to room temperature, and 150 ml of water was added to the reaction system. The mixture was extracted with EA. The organic phase was washed with saturated sodium chloride solution, dried over anhydrous sodium sulfate and filtered. The filtrate was concentrated under reduced pressure, and then the residue was separated and purified by column chromatography (eluent: PE/EA=1/3) to obtain compound 38F (2.3 g, 56%).

LC-MS (ESI): m/z=361, 363 [M+H]⁺.

Step 6:

### (S)-methyl 4-(5-((tetrahydrofuran-3-yl)oxy)benzo[d]oxazol-2-yl)picolinate (38G)

Compound 38F (2 g, 5.54 mmol), [1,1′-bis(diphenylphosphino)ferrocene]palladium dichloride dichloromethane complex (200 mg) and triethylamine (1.68 g, 16.6 mmol) were added to a mixed system of methanol (30 mL) and dichloromethane (15 ml), and the mixture was reacted at 120° C. under carbon monoxide (30 MPa) for 3 hours. The reaction solution was cooled to room temperature and filtered. The filtrate was concentrated under reduced pressure, and then the residue was separated and purified by column chromatography (eluent: PE/EA=1/3) to obtain compound 38G (1.7 g, 93%).

LC-MS (ESI): m/z=341.1 [M+H]⁺.

Step 7:

### (S)-4-(5-((tetrahydrofuran-3-yl)oxy)benzo[d]oxazol-2-yl)picolinic Acid (38H)

Compound 38G (1.7 g, 5 mmol) and sodium hydroxide (0.67 g, 16.8 mmol) were added to a mixed system of methanol (20 mL) and water (4 ml), and the mixture was reacted at room temperature for 2 hours. The reaction solution was adjusted to pH=1-2 with dilute hydrochloric acid, and a small amount of acetone was added to the reaction solution. The mixture was filtered, and the filtered solid was washed with water to obtain compound 38H (1.1 g, 67%).

LC-MS (ESI): m/z=327.1 [M+H]⁺.

Step 8:

### (4-((2-methyl-2H-tetrazol-5-yl)(phenyl)methyl)piperazin-1-yl)(4-(5-(((S)-tetrahydrofuran-3-yl)oxy)benzo[d]oxazol-2-yl)pyridin-2-yl)methanone (Compound 38)

Intermediate 2 (60 mg, 0.234 mmol) was dissolved in N,N-dimethylformamide (2 mL), and the mixture was cooled to 0° C.; HATU (95 mg, 0.25 mmol) and DIPEA (75 mg, 0.585 mmol) were added; and then compound 38H (80 mg 0.25 mmol) was added. After the addition, the mixture was stirred for 1 hour while the temperature was controlled at 0° C., and then the reaction was quenched by adding ice water (10 mL). The resulting solution was extracted twice with dichloromethane (50 mL×2). The organic phase was combined, dried over anhydrous sodium sulfate and concentrated under reduced pressure, and then the residue was separated and purified by column chromatography (eluent: DCM/MeOH=1/20) to obtain compound 38 (50 mg, 38%).

¹H NMR (400 MHz, CD₃OD) δ 8.77-8.76 (m, 1H), 8.28-8.26 (m, 1H), 8.18-8.16 (m, 1H), 7.64-7.62 (m, 1H), 7.53-7.51 (m, 2H), 7.34-7.28 (m, 4H), 7.11-7.06 (m, 1H), 5.11-5.09 (m, 1H), 5.01 (s, 1H), 4.35 (s, 3H), 4.03-3.97 (m, 3H), 3.92-3.88 (m, 1H), 3.85-3.83 (m, 2H), 3.58-3.55 (m, 2H), 2.70-2.62 (m, 1H), 2.60-2.49 (m, 2H), 2.47-2.37 (m, 1H), 2.35-2.26 (m, 1H), 2.20-2.10 (m, 1H).

LC-MS (ESI): m/z=567.2[M+H]⁺.

### Example 39

### (4-((2-(difluoromethyl)-2H-tetrazol-5-yl)(phenyl)methyl)piperazin-1-yl)(4-(5-(((S)-tetrahydrofuran-3-yl)oxy)benzo[d]oxazol-2-yl)pyridin-2-yl)methanone

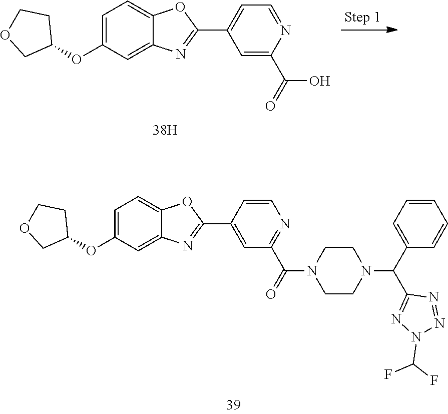 [SMILES: O=C(O)c1cc(-c2nc3cc(O[C@H]4CCOC4)ccc3o2)ccn1 ; O=C(c1cc(-c2nc3cc(O[C@H]4CCOC4)ccc3o2)ccn1)N1CCN(C(c2ccccc2)c2nnn(C(F)F)n2)CC1]

Step 1:

### (4-((2-(difluoromethyl)-2H-tetrazol-5-yl)(phenyl)methyl)piperazin-1-yl)(4-(5-(((S)-tetrahydrofuran-3-yl)oxy)benzo[d]oxazol-2-yl)pyridin-2-yl)methanone (Compound 39)

Intermediate 6 (70 mg, 0.236 mmol) was dissolved in N,N-dimethylformamide (2 mL), and the mixture was cooled to 0° C.; HATU (95 mg, 0.25 mmol) and DIPEA (75 mg, 0.585 mmol) were added; and then compound 38H (70 mg 0.215 mmol) was added. After the addition, the mixture was stirred for 1 hour while the temperature was controlled at 0° C., and then the reaction was quenched by adding ice water (10 mL). The resulting solution was extracted twice with dichloromethane (50 mL×2). The organic phase was combined, dried over anhydrous sodium sulfate and concentrated under reduced pressure, and then the residue was separated and purified by column chromatography (eluent: DCM/MeOH=1/20) to obtain compound 39 (50 mg, 39%).

¹H NMR (400 MHz, CD₃OD) δ 8.77-8.76 (m, 1H), 8.30-8.02 (m, 3H), 7.63-7.61 (m, 1H), 7.54-7.52 (m, 2H), 7.38-7.30 (m, 4H), 7.10-7.06 (m, 1H), 5.19 (s, 1H), 5.11-5.08 (m, 1H), 4.03-3.95 (m, 3H), 3.92-3.83 (m, 3H), 3.59-3.57 (m, 2H), 2.73-2.68 (m, 1H), 2.63-2.52 (m, 2H), 2.46-2.41 (m, 1H), 2.35-2.26 (m, 1H), 2.18-2.12 (m, 1H).

LC-MS (ESI): m/z=603.2[M+H]⁺.

### Example 40

### N-(2-(2-(4-((2-(difluoromethyl)-2H-tetrazol-5-yl)(phenyl)methyl)piperazine-1-carbonyl)pyridin-4-yl)benzo[d]oxazol-5-yl)acetamide

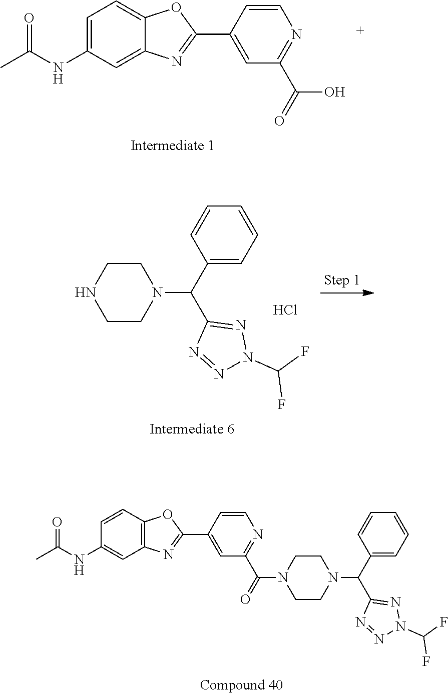 [SMILES: CC(=O)Nc1ccc2oc(-c3ccnc(C(=O)O)c3)nc2c1 ; "+" ; FC(F)n1nnc(C(c2ccccc2)N2CCNCC2)n1 ; Cl ; CC(=O)Nc1ccc2oc(-c3ccnc(C(=O)N4CCN(C(c5ccccc5)c5nnn(C(F)F)n5)CC4)c3)nc2c1]

Step 1:

### N-(2-(2-(4-((2-(difluoromethyl)-2H-tetrazol-5-yl)(phenyl)methyl)piperazine-1-carbonyl)pyridin-4-yl)benzo[d]oxazol-5-yl)acetamide (Compound 40)

Intermediate 1 (75 mg, 0.253 mmol) was dissolved in N,N-dimethylformamide (2 mL), and the mixture was cooled to 0° C.; HATU (96 mg, 0.253 mmol) and DIPEA (98 mg, 0.76 mmol) were added; and then compound intermediate 6 (70 mg, 0.21 mmol) was added. After the addition, the mixture was stirred for 1 hour while the temperature was controlled at 0° C., and then the reaction was quenched by adding ice water (10 mL). The resulting solution was extracted twice with dichloromethane (50 mL×2). The organic phase was combined, dried over anhydrous sodium sulfate and concentrated under reduced pressure, and then the residue was separated and purified by column chromatography (eluent: MeOH/DCM=1/20) to obtain compound 40 (45 mg, 37%).

¹H NMR (400 MHz, CD₃OD) δ8.78 (s, 1H), 8.34-8.05 (m, 4H), 7.67-7.65 (m, 1H), 7.60-7.54 (m, 3H), 7.44-7.36 (m, 3H), 5.48 (5, 1H), 4.95-3.90 (m, 2H), 3.73-3.68 (m, 2H), 2.89-2.72 (m, 4H), 2.17 (s, 3H).

LC-MS (ESI): m/z=574.2[M+H]⁺.

### Example 41

### (S/R)—N-(2-(2-(1-((2-(difluoromethyl)-2H-tetrazol-5-yl)(phenyl)methyl)piperidine-4-carbonyl)pyridin-4-yl)benzo[d]oxazol-5-yl)acetamide

### Example 42

### (R/S)—N-(2-(2-(1-((2-(difluoromethyl)-2H-tetrazol-5-yl)(phenyl)methyl)piperidine-4-carbonyl)pyridin-4-yl)benzo[d]oxazol-5-yl)acetamide

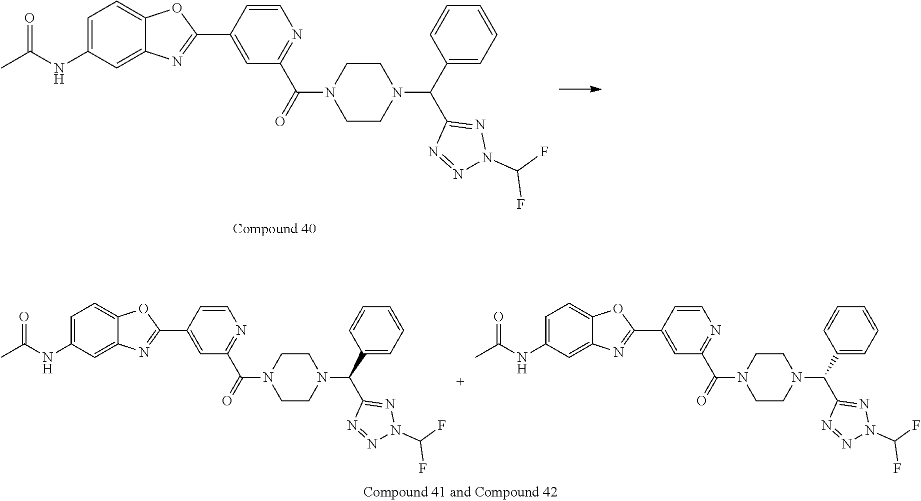

Compound 40 (490 mg, 0.86 mmol) was subject to chiral preparative separation to obtain compound 41 (222 mg, 45%) and compound 42 (221 mg, 45%).

Preparative chromatography conditions: chromatographic column: ChiralPak AD, 10 μm, 250×30 mm; mobile phase system: carbon dioxide/ethanol=60/40 (v) (0.05% DEA); isocratic elution for 15 min. flow rate: 80 ml/min. Compound 41 (retention time: about 6.50 min, 222 mg, a compound in a single configuration) and compound 42 (retention time: about 10.77 min, 221 mg, a compound in a single configuration) were obtained.

Compound 41: ¹H NMR (400 MHz, MeOD) δ 8.78-8.77 (m, 1H), 8.32-8.00 (m, 4H), 7.67-7.65 (m, 1H), 7.58-7.50 (m, 3H), 7.41-7.28 (m, 3H), 5.19 (s, 1H), 3.86-3.84 (m, 2H), 3.60-3.54 (m, 2H), 2.76-2.67 (m, 1H), 2.65-2.50 (m, 2H), 2.48-2.39 (m, 1H), 2.16 (s, 3H).

LC-MS (ESI): 573.2[M+H]⁺.

Compound 42: ¹H NMR (400 MHz, MeOD) δ 8.78-8.77 (m, 1H), 8.32-8.01 (m, 4H), 7.67-7.65 (m, 1H), 7.59-7.50 (m, 3H), 7.40-7.28 (m, 3H), 5.19 (s, 1H), 3.86-3.84 (m, 2H), 3.62-3.55 (m, 2H), 2.74-2.68 (m, 1H), 2.65-2.51 (m, 2H), 2.47-2.43 (m, 1H), 2.17 (s, 3H).

LC-MS (ESI): 573.2[M+H]⁺.

### Example 43

### (4-((2-methyl-2H-tetrazol-5-yl)(phenyl)methyl)piperazin-1-yl)(4-(5-(((R)-tetrahydrofuran-3-yl)oxy)benzo[d]oxazol-2-yl)pyridin-2-yl)methanone

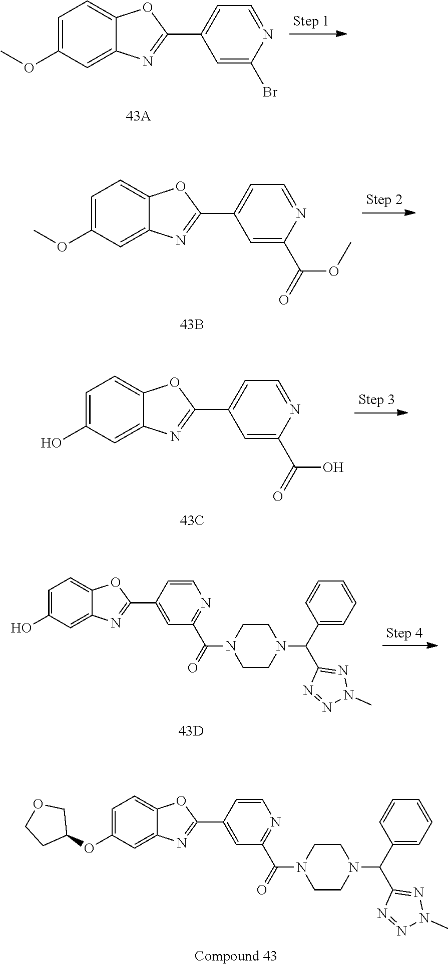

Step 1:

### Methyl 4-(5-methoxybenzo[d]oxazol-2-yl)picolinate (43B)

Compound 43A (4 g, 13.1 mmol), [1,1′-bis(diphenylphosphino)ferrocene]palladium dichloride dichloromethane complex (400 mg) and triethylamine (4 g, 39.3 mmol) were added to a mixed system of methanol (40 mL) and dichloromethane (15 ml), and the mixture was reacted at 120° C. under carbon monoxide (30 MPa) for 3 hours. The reaction solution was cooled to room temperature and filtered. The filtrate was concentrated under reduced pressure, and then the residue was separated and purified by column chromatography (eluent: PE/DCM=1/1) to obtain compound 43B (3.4 g, 91%).

LC-MS (ESI): m/z=385.1 [M+H]⁺.

Step 2:

### 4-(5-hydroxybenzo[d]oxazol-2-yl)picolinic Acid (43C)

Compound 43B (3.4 g, 12 mmol) was added to dichloromethane (40 mL), and boron tribromide (60 mmol, 60 ml) was added dropwise at 0° C. The reaction was stirred at room temperature for 2 hours. The reaction was quenched with methanol and then adjusted to pH=7-8 with aqueous sodium bicarbonate solution. The solid undissolved in the system was filtered directly, and the filtered solid was washed with water and dried to obtain compound 43C (2.2 g, 72%).

LC-MS (ESI): m/z=257.1 [M+H]⁺.

Step 3:

### (4-(5-hydroxybenzo[d]oxazol-2-yl)pyridin-2-yl)(4-((2-methyl-2H-tetrazol-5-yl)(phenyl)methyl)piperazin-1-yl)methanone (43D)

Intermediate 1 (400 mg, 1.55 mmol) was dissolved in N,N-dimethylformamide (5 mL), and the mixture was cooled to 0° C.; HATU (706 mg, 1.86 mmol) and DIPEA (500 mg, 3.88 mmol) were added; and then compound 43C (476 mg, 1.86 mmol) was added. After the addition, the mixture was stirred for 1 hour while the temperature was controlled at 0° C., and then the reaction was quenched by adding water (10 mL). The resulting solution was extracted twice with ethyl acetate (50 mL×2). The organic phase was combined, dried over anhydrous sodium sulfate and concentrated under reduced pressure, and then the residue was separated and purified by column chromatography (eluent: DCM/MeOH=1/20) to obtain 43D (600 mg, 78%).

LC-MS (ESI): m/z=497.2 [M+H]⁺.

Step 6:

### (4-((2-methyl-2H-tetrazol-5-yl)(phenyl)methyl)piperazin-1-yl)(4-(5-(((R)-tetrahydrofuran-3-yl)oxy)benzo[d]oxazol-2-yl)pyridin-2-yl)methanone (Compound 43)

Compound 43D (200 mg, 0.4 mmol), (S)-tetrahydrofuran-3-yl methanesulfonate (89 mg, 0.52 mmol) and cesium carbonate (260 mg, 0.8 mmol) were added to N,N-dimethylformamide (5 mL), and the mixture was reacted at 90° C. for 2 hours. The reaction solution was cooled to room temperature, and 30 ml of water was added to the reaction system. The mixture was extracted with ethyl acetate. The organic phase was washed with saturated sodium chloride solution. The filtrate was concentrated under reduced pressure, and then the residue was separated and purified by column chromatography (eluent: DCM/MeOH=1/25) to obtain compound 43 (100 mg, 44%).

¹H NMR (400 MHz, MeOD) δ 8.77-8.76 (m, 1H), 8.28-8.27 (m, 1H), 8.17-8.15 (m, 1H), 7.62 (d, 1H), 7.54-7.49 (m, 2H), 7.37-7.25 (m, 4H), 7.10-7.07 (m, 1H), 5.12-5.08 (m, 1H), 5.01 (s, 1H), 4.35 (s, 3H), 4.03-3.94 (m, 3H), 3.92-3.87 (m, 1H), 3.85-3.82 (m, 2H), 3.56 (t, 2H), 2.70-2.62 (m, 1H), 2.60-2.49 (m, 2H), 2.46-2.38 (m, 1H), 2.35-2.26 (m, 1H), 2.20-2.11 (m, 1H).

LC-MS (ESI): m/z=567.2[M+H]⁺.

### Example 44

### (4-(5-(difluoromethoxy)benzo[d]oxazol-2-yl)pyridin-2-yl)(4-((2-methyl-2H-tetrazol-5-yl)(phenyl)methyl)piperazin-1-yl)methanone

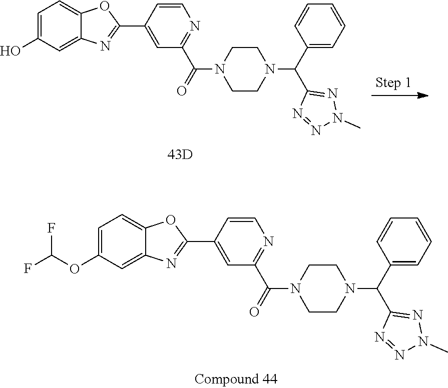 [SMILES: Cn1nnc(C(c2ccccc2)N2CCN(C(=O)c3cc(-c4nc5cc(O)ccc5o4)ccn3)CC2)n1 ; Cn1nnc(C(c2ccccc2)N2CCN(C(=O)c3cc(-c4nc5cc(OC(F)F)ccc5o4)ccn3)CC2)n1]

Step 1:

### (4-(5-(difluoromethoxy)benzo[d]oxazol-2-yl)pyridin-2-yl)(4-((2-methyl-2H-tetrazol-5-yl)(phenyl)methyl)piperazin-1-yl)methanone (compound 44)

Compound 43D (250 mg, 0.5 mmol) was added to acetonitrile (3 mL) and water (3 mL), and the mixture was cooled to 0° C.; and then potassium hydroxide (560 mg, 10 mmol) was added. The mixture was stirred for 10 min, and then diethyl (bromodifluoromethyl)phosphonate (267 mg, 1 mmol) was added. After the addition, the mixture was stirred for 1 hour while the temperature was controlled at 0° C., and then water (10 mL) was added. The resulting solution was extracted twice with ethyl acetate (20 mL×2). The organic phase was combined, dried over anhydrous sodium sulfate and concentrated under reduced pressure, and then the residue was separated and purified by column chromatography (eluent: DCM/MeOH=1/25) to obtain compound 44 (120 mg, 44%).

¹H NMR (400 MHz, MeOD) δ 8.80-8.79 (m, 1H), 8.32-8.31 (m, 1H), 8.21-8.19 (m, 1H), 7.76 (d, 1H), 7.61 (d, 1H), 7.55-7.49 (m, 2H), 7.38-7.24 (m, 4H), 6.88 (t, 1H), 5.01 (s, 1H), 4.35 (s, 3H), 3.86-3.84 (m, 2H), 3.58-3.56 (m, 2H), 2.69-2.64 (m, 1H), 2.60-2.48 (m, 2H), 2.47-2.37 (m, 1H).

LC-MS (ESI): m/z=547.2[M+H]⁺.

### Example 45

### (R/S)-1-(2-(2-(4-((2-methyl-2H-tetrazol-5-yl)(phenyl)methyl)piperazine-1-carbonyl)pyridin-4-yl)benzo[d]oxazol-5-yl)pyrrolidin-2-one

### Example 46

### (S/R)-1-(2-(2-(4-((2-methyl-2H-tetrazol-5-yl)(phenyl)methyl)piperazine-1-carbonyl)pyridin-4-yl)benzo[d]oxazol-5-yl)pyrrolidin-2-one

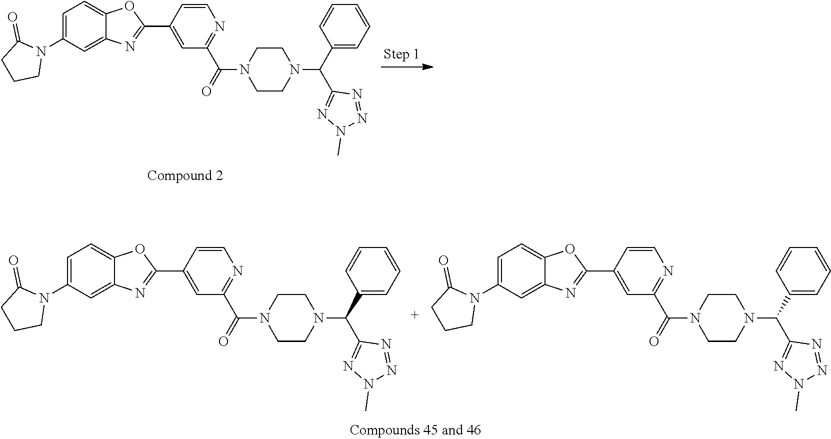 [SMILES: Cn1nnc(C(c2ccccc2)N2CCN(C(=O)c3cc(-c4nc5cc(N6CCCC6=O)ccc5o4)ccn3)CC2)n1 ; Cn1nnc([C@H](c2ccccc2)N2CCN(C(=O)c3cc(-c4nc5cc(N6CCCC6=O)ccc5o4)ccn3)CC2)n1 ; Cn1nnc([C@@H](c2ccccc2)N2CCN(C(=O)c3cc(-c4nc5cc(N6CCCC6=O)ccc5o4)ccn3)CC2)n1]

Compound 2 (810 mg, 1.44 mmol) was subjected to chiral separation by using a chiral chromatographic column, with purification conditions as follows: instrument: Waters 80 preparative SFC (SFC-17) (preparative liquid phase chromatographic instrument); chromatographic column: ChiralCel OJ, 250×30 mm, inner diameter: 10 μm. The sample was dissolved in methanol and filtered with a 0.45 m filter to prepare a sample solution. Preparative chromatography conditions: mobile phase system: A: CO₂, B: methanol (0.1% NH₃H₂O); gradient B: 55%. flow rate: 80 ml/min. Compound 45 (retention time: about 7.856 min, 0.21 g) and compound 46 (retention time: about 8.555 min, 0.22 g) were obtained.

Compound 45: ¹H NMR (400 MHz, CD₃OD) δ 8.80-8.76 (m, 1H), 8.32 (s, 1H), 8.15-8.13 (m, 1H), 8.05 (d, 1H), 7.76-7.72 (m, 2H), 7.56-7.52 (m, 2H), 7.38-7.27 (m, 3H), 5.04 (s, 1H), 4.36 (s, 3H), 4.05-3.98 (m, 2H), 3.84-3.83 (m, 2H), 3.58-3.56 (m, 2H), 2.66-2.44 (m, 6H), 2.27-2.19 (m, 2H).

LC-MS (ESI): m/z=564.2 [M+H]⁺.

Compound 46: ¹H NMR (400 MHz, CD₃OD) δ 8.79-8.77 (m, 1H), 8.31 (s, 1H), 8.14-8.13 (m, 1H), 8.06 (d, 1H), 7.77-7.72 (m, 2H), 7.54-7.52 (m, 2H), 7.35-7.27 (m, 3H), 5.05 (s, 1H), 4.35 (s, 3H), 4.03-3.99 (m, 2H), 3.84-3.83 (m, 2H), 3.59-3.57 (m, 2H), 2.66-2.44 (m, 6H), 2.26-2.19 (m, 2H).

LC-MS (ESI): m/z=564.2 [M+H]⁺.

### Example 47

### N-(2-(2-(4-hydroxy-4-((2-methyl-2H-tetrazol-5-yl)(phenyl)methyl)piperidine-1-carbonyl)pyridin-4-yl)benzo[d]oxazol-5-yl)acetamide

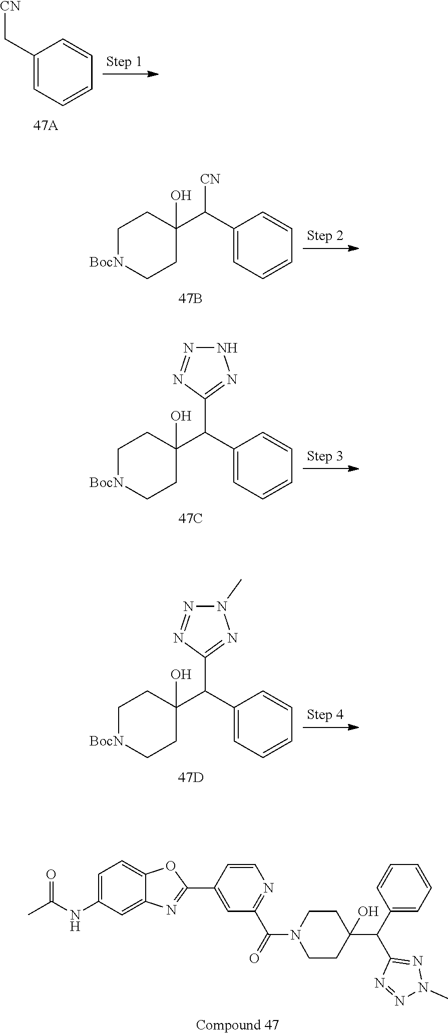 [SMILES: N#CCc1ccccc1 ; CC(C)(C)OC(=O)N1CCC(O)(C(C#N)c2ccccc2)CC1 ; CC(C)(C)OC(=O)N1CCC(O)(C(c2ccccc2)c2nn[nH]n2)CC1 ; Cn1nnc(C(c2ccccc2)C2(O)CCN(C(=O)OC(C)(C)C)CC2)n1 ; CC(=O)Nc1ccc2oc(-c3ccnc(C(=O)N4CCC(O)(C(c5ccccc5)c5nnn(C)n5)CC4)c3)nc2c1]

Step 1:

### tert-butyl 4-(cyano(phenyl)methyl)-4-hydroxypiperidine-1-carboxylate (47B)

Phenylacetonitrile 47A (10.00 g, 85.4 mmol) was dissolved in anhydrous THE (100 mL) at room temperature and cooled to −78° C. under nitrogen protection. Then a solution of n-butyl lithium in n-hexane (37.6 mL, 93.9 mmol) was added dropwise, and the mixture was reacted at −78° C. for 30 minutes; A solution of N-tert-butoxycarbonyl-4-piperidone (18.70 g, 93.9 mmol) in THE (100 mL) was added dropwise at −78° C. After the dropwise addition was completed, the mixture was allowed to naturally warm to room temperature and reacted for another 2 hours. The reaction was quenched by adding saturated ammonium chloride (100 mL) and extracted with ethyl acetate (100 mL×2). The combined organic phase was dried over anhydrous sodium sulfate and concentrated under reduced pressure, and the residue was separated and purified by column chromatography (PE:EA=10:1 to 5:1) to obtain 47B (17.00 g, 63.0%).

LC-MS (ESI): m/z=317.2[M+H]⁺.

Step 2:

### tert-butyl-4-hydroxy-4-(phenyl(2H-tetrazol-5-yl)methyl)piperidine-1-carboxylate (47C)

47B (3.22 g, 10.0 mmol) and triethylamine hydrochloride (4.13 g, 30.0 mmol) were added to toluene (100 mL) at room temperature; then sodium azide (1.95 g, 30.0 mmol) was added; and the mixture was warmed to 100° C. and reacted overnight. The reaction solution was cooled to room temperature, adjusted to pH 3-4 by dropwise adding dilute hydrochloric acid (1 M) and extracted with ethyl acetate (50 mL×2). The organic phase was combined, washed with water (60 mL×2) and saturated sodium chloride solution (60 mL×1) and concentrated under reduced pressure to obtain a crude product of 47C (3.25 g).

LC-MS (ESI): m/z=360.2[M+H]⁺.

Step 3:

### tert-buty-4-hydroxy-4-((2-methyl-2H-tetrazol-5-yl)(phenyl)methyl)piperidine-1-carboxylate (47D)

The crude product of 47C (3.25 g) obtained in step 2 was dissolved in tetrahydrofuran (40 mL) and methanol (10 mL) at room temperature. Then a solution of trimethylsilyldiazomethane in n-hexane (6.8 mL, 13.5 mmol) was added dropwise, and the mixture was stirred at room temperature for another 3 h. The reaction solution was subjected to rotary evaporation and then column chromatography (PE:EA=10:1) to obtain 47D (3.12 g, yield for two steps: 83.5%).

Step 4:

### N-(2-(2-(4-hydroxy-4-((2-methyl-2H-tetrazol-5-yl)(phenyl)methyl)piperidine-1-carbonyl)pyridin-4-yl)benzo[d]oxazol-5-yl)acetamide

47D was used as a starting material to obtain compound 47 according to the synthetic step of compound 1.

LC-MS (ESI): m/z=553.3 [M+H]⁺.

### Example 48

### N-(2-(2-(4-fluoro-4-((2-methyl-2H-tetrazol-5-yl)(phenyl)methyl)piperidine-1-carbonyl)pyridin-4-yl)benzo[d]oxazol-5-yl)acetamide

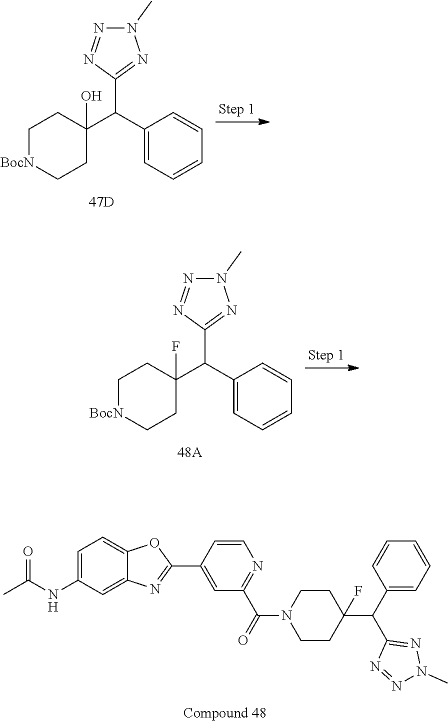 [SMILES: Cn1nnc(C(c2ccccc2)C2(O)CCN(C(=O)OC(C)(C)C)CC2)n1 ; Cn1nnc(C(c2ccccc2)C2(F)CCN(C(=O)OC(C)(C)C)CC2)n1 ; CC(=O)Nc1ccc2oc(-c3ccnc(C(=O)N4CCC(F)(C(c5ccccc5)c5nnn(C)n5)CC4)c3)nc2c1]

Step 1:

### tert-butyl 4-fluoro-4-((2-methyl-2H-tetrazol-5-yl)(phenyl)methyl)piperidine-1-carboxylate (48A)

47D (373 mg, 1.0 mmol) was dissolved in anhydrous dichloromethane (5 mL) at room temperature. DAST (320 mg, 2.0 mmol) was added dropwise at −78° C. under nitrogen protection. After the dropwise addition was completed, the mixture was allowed to naturally warm to room temperature and reacted overnight. 0.5 mL of water was added dropwise, and then the reaction solution was subjected to rotary evaporation and column chromatography to obtain a crude product of 48A (250 mg), which was directly used in the next reaction without purification.

LC-MS (ESI): m/z=320.2 [M−55]⁺.

Step 2:

### N-(2-(2-(4-fluoro-4-((2-methyl-2H-tetrazol-5-yl)(phenyl)methyl)piperidine-1-carbonyl)pyridin-4-yl)benzo[d]oxazol-5-yl)acetamide (Compound 48)

48A was used as a starting material to obtain compound 48 according to the synthetic step of compound 1.

LC-MS (ESI): m/z=555.2 [M+1]⁺.

### Example 49

### N-(2-(2-(4-((2-methyl-2H-tetrazol-5-yl)(phenyl)methylene)piperidine-1-carbonyl)pyridin-4-yl)benzo[d]oxazol-5-yl)acetamide

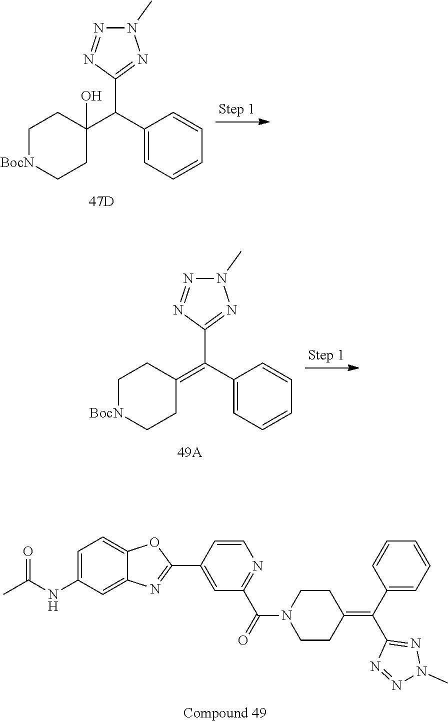 [SMILES: Cn1nnc(C(c2ccccc2)C2(O)CCN(C(=O)OC(C)(C)C)CC2)n1 ; Cn1nnc(C(=C2CCN(C(=O)OC(C)(C)C)CC2)c2ccccc2)n1 ; CC(=O)Nc1ccc2oc(-c3ccnc(C(=O)N4CCC(=C(c5ccccc5)c5nnn(C)n5)CC4)c3)nc2c1]

Step 1:

### tert-butyl-4-((2-methyl-2H-tetrazol-5-yl)(phenyl)methylene)piperidine-1-carboxylate (49A)

47D (373 mg, 1.0 mmol) was dissolved in pyridine (5 mL) at room temperature. SOCl₂ (2.0 ml) was added dropwise at 0° C. under nitrogen protection. After the dropwise addition was completed, the mixture was allowed to naturally warm to room temperature and reacted for 1 h. The reaction solution was subjected to rotary evaporation and column chromatography to obtain a crude product of 49A (250 mg), which was directly used in the next reaction without purification.

LC-MS (ESI): m/z=356.2 [M+H]⁺.

Step 2:

### N-(2-(2-(4-((2-methyl-2H-tetrazol-5-yl)(phenyl)methylene)piperidine-1-carbonyl)pyridin-4-yl)benzo[d]oxazol-5-yl)acetamide (Compound 49)

49A was used as a starting material to obtain compound 49 according to the synthetic step of compound 1.

LC-MS (ESI): m/z=535.2 [M+H]⁺.

### Example 50

### 3-(2-(2-(4-((2-methyl-2H-tetrazol-5-yl)(phenyl)methyl)piperazine-1-carbonyl)pyridin-4-yl)benzo[d]oxazol-5-yl)oxazolidin-2-one

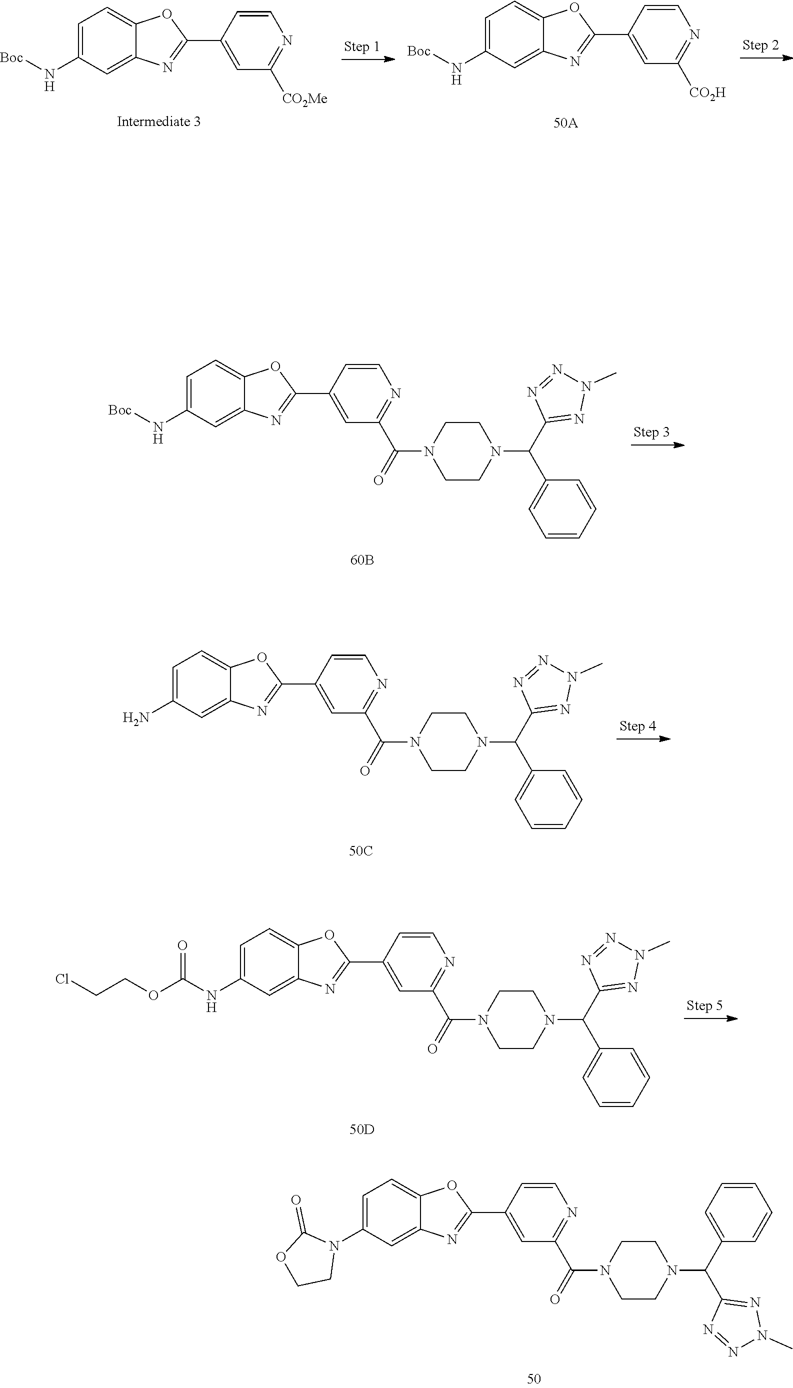 [SMILES: COC(=O)c1cc(-c2nc3cc(NC(=O)OC(C)(C)C)ccc3o2)ccn1 ; CC(C)(C)OC(=O)Nc1ccc2oc(-c3ccnc(C(=O)O)c3)nc2c1 ; Cn1nnc(C(c2ccccc2)N2CCN(C(=O)c3cc(-c4nc5cc(NC(=O)OC(C)(C)C)ccc5o4)ccn3)CC2)n1 ; Cn1nnc(C(c2ccccc2)N2CCN(C(=O)c3cc(-c4nc5cc(N)ccc5o4)ccn3)CC2)n1 ; Cn1nnc(C(c2ccccc2)N2CCN(C(=O)c3cc(-c4nc5cc(NC(=O)OCCCl)ccc5o4)ccn3)CC2)n1 ; Cn1nnc(C(c2ccccc2)N2CCN(C(=O)c3cc(-c4nc5cc(N6CCOC6=O)ccc5o4)ccn3)CC2)n1]

Step 1:

### 4-(5-((tert-butoxycarbonyl)amino)benzo[d]oxazol-2-yl)picolinic Acid (5A)

Intermediate 3 was used as a raw material to obtain compound 50A according to the synthetic step (step 5) of intermediate 1.

LC-MS (ESI): m/z=356.1 [M+H]⁺.

Step 2:

### tert-butyl (2-(2-(4-((2-methyl-2H-tetrazol-5-yl)(phenyl)methyl)piperazine-1-carbonyl)pyridin-4-yl)benzo[d]oxazol-a-yl)carbamate (50B)

Compound 50A was used as a raw material to obtain compound 50B according to the synthetic step (step 5) of compound 1.

LC-MS (ESI): m/z=496.3 [M+H]⁺.

Step 3:

### (4-(2-aminobenzo[d]oxazol-2-yl)pyridin-2-yl)(4-((2-methyl-2H-tetrazol-5-yl)(phenyl)methyl)piperazin-1-yl)methanone (50C)

Compound 50B was used as a raw material to obtain compound C according to the synthetic step (step 1) of intermediate 4.

LC-MS (ESI): m/z=496.2 [M+H]⁺.

Step 4:

### 2-chloroethyl (2-(2-(4-((2-methyl-2H-tetrazol-5-yl)(phenyl)methyl)piperazine-1-carbonyl)pyridin-4-yl)benzo[d]oxazol-5-yl)carbamate (50D)

50C (50 mg, 0.10 mmol) was added to a 50 ml round bottom flask and dissolved in DCM, and then chloroethyl chloroformate (14 mg, 0.1 mmol) and triethylamine (26 mg, 0.2 mmol) were added. The mixture was reacted at room temperature for 2 h, and then water (10 mL) was added. The resulting reaction solution was extracted twice with dichloromethane (30 mL×2). The organic phase was combined, dried and concentrated to obtain compound 50D (50 mg, 82%).

LC-MS (ESI): m/z=602.2 [M+H]⁺.

Step 5:

### 3-(2-(2-(4-((2-methyl-2H-tetrazol-5-yl)(phenyl)methyl)piperazine-1-carbonyl)pyridin-4-yl)benzo[d]oxazol-5-yl)oxazolidin-2-one (Compound 50)

50D (50 mg, 0.08 mmol) was added to a 50 ml round bottom flask and dissolved in DMF (3 mL); then NaH (7 mg, 0.16 mmol) was added; and the mixture was reacted at room temperature for 3 h. Water (10 mL) was added, and the resulting solution was extracted with ethyl acetate (30 mL×2). The organic phase was combined, dried and concentrated, and then the residue was separated and purified by HPLC, wherein the preparation conditions were as follows: instrument: waters 2767 (preparative liquid phase chromatographic instrument); chromatographic column: XBridge C18 5 μm, 19*250 mm. The sample was dissolved in acetonitrile and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 5% to 50%; time: 15 min; flow rate: 12 ml/min. Compound 50 (3 mg, 9%) was obtained using components with retention time of 12.2 min.

¹H NMR (400 MHz, CDCl₃) δ 8.73 (d, 1H), 8.49 (s, 1H), 8.16 (d, 1H), 7.89-7.77 (m, 4H), 7.63 (d, 1H), 7.47-7.48 (m, 3H), 5.84 (s, 1H), 4.53-4.57 (m, 2H), 4.41 (s, 3H), 4.14-4.18 (m, 6H), 3.39-3.49 (m, 2H), 3.17-3.18 (m, 2H).

LC-MS (ESI): m/z=566.2 [M+H]⁺.

### Example 51

### Methyl (2-(2-(4-((2-methyl-2H-tetrazol-5-yl)(phenyl)methyl)piperazine-1-carbonyl)pyridin-4-yl)benzo[d]oxazol-5-yl)carbamate

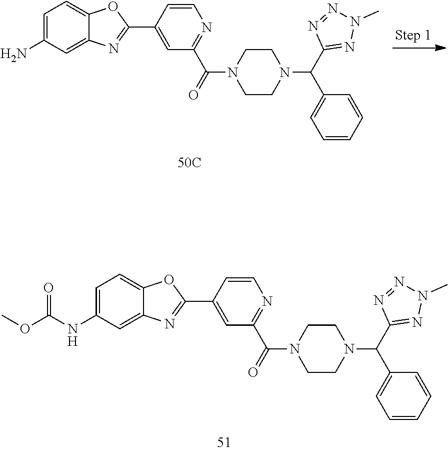

Step 1:

### Methyl (2-(2-(4-((2-methyl-2H-tetrazol-5-yl)(phenyl)methyl)piperazine-1-carbonyl)pyridin-4-yl)benzo[d]oxazol-5-yl)carbamate (Compound 51)

To a 100 ml round bottom flask, 50C (50 mg, 0.10 mmol) was added and dissolved in DCM (5 mL); triphosgene (52 mg, 0.2 mmol) and DIEA (26 mg, 0.20 mmol) were added; and the mixture was reacted at room temperature for 3 h. Then methanol (5 mL) was added, and the resulting mixture was reacted at room temperature for 1 h. The reaction solution was concentrated, and then the residue was separated and purified by HPLC, wherein the preparation conditions were as follows: instrument: waters 2767 (preparative liquid phase chromatographic instrument); chromatographic column: XBridge C18 5 μm, 19*250 mm. The sample was dissolved in acetonitrile and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 5% to 50%; time: 15 min; flow rate: 12 ml/min. Compound 51 (7 mg, 13%) was obtained using components with retention time of 10.6 min.

¹H NMR (400 MHz, CDCl₃) δ 8.73 (d, 1H), 8.49 (s, 1H), 8.17 (d, 1H), 7.93 (s, 1H), 7.79-7.81 (m, 2H), 7.43-7.57 (m, 5H), 6.77 (s, 1H), 6.86 (s, 1H), 4.43 (s, 3H), 4.18-4.24 (m, 4H), 3.83 (s, 3H), 3.52-3.55 (m, 2H), 3.17-3.19 (m, 2H).

LC-MS (ESI): m/z=554.2 [M+H]⁺.

### Example 52

### 1-methyl-3-(2-(2-(4-((2-methyl-2H-tetrazol-5-yl)(phenyl)methyl)piperazine-1-carbonyl)pyridin-4-yl)benzo[d]oxazol-5-yl)urea

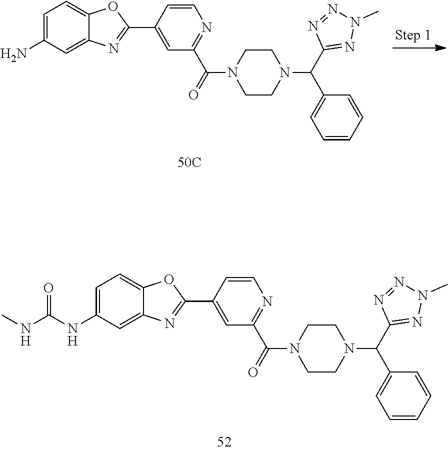

Step 1:

### 1-methyl-3-(2-(2-(4-((2-methyl-2H-tetrazol-5-yl)(phenyl)methyl)piperazine-1-carbonyl)pyridin-4-yl)benzo[d]oxazol-5-yl) urea (Compound 52)

To a 100 ml round bottom flask, 50C (50 mg, 0.10 mmol) was added and dissolved in DCM (5 mL); triphosgene (13 mg, 0.05 mmol) and DIEA (52 mg, 0.40 mmol) were added; and the mixture was reacted at room temperature for 1 h. Then a solution of methylamine in tetrahydrofuran (1 mL) was added, and the resulting mixture was reacted at room temperature for 1 h. The reaction solution was concentrated, and then the residue was separated and purified by HPLC, wherein the preparation conditions were as follows: instrument: waters 2767 (preparative liquid phase chromatographic instrument); chromatographic column: XBridge C18 5 μm, 19*250 mm. The sample was dissolved in acetonitrile and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 5% to 50%; time: 15 min; flow rate: 12 ml/min. Compound 52 (11 mg, 19%) was obtained using components with retention time of 11.5 min.

¹H NMR (400 MHz, CDCl₃) δ 8.72 (d, 1H), 8.42 (s, 1H), 8.11 (m, 1H), 7.75-7.67 (m, 2H), 7.55 (d, 1H), 7.45-7.41 (m, 2H), 7.37 (m, 1H), 7.13 (s, 1H), 5.62 (s, 1H), 5.30 (s, 1H), 4.40 (s, 3H), 4.17-3.96 (m, 5H), 3.26 (s, 2H), 2.95 (d, 2H), 2.88 (s, 3H).

LC-MS (ESI): m/z=553.3 [M+H]⁺.

### Example 53

### N-(2-(2-(6-((2-methyl-2H-tetrazol-5-yl)(phenyl)methyl)-3,6-diazabicyclo[3.1.0]hexane-3-carbonyl)pyridin-4-yl)benzo[d]oxazol-5-yl)acetamide

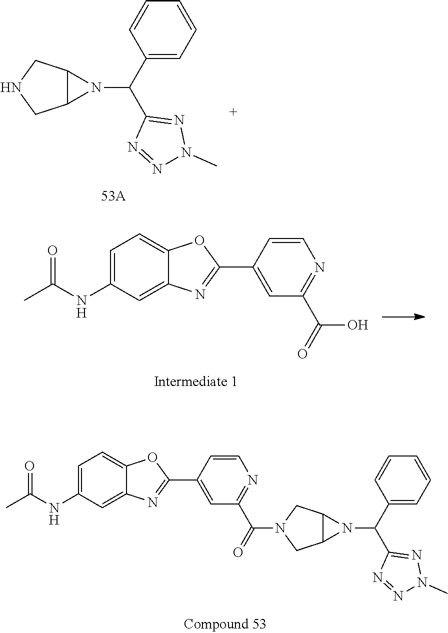 [SMILES: Cn1nnc(C(c2ccccc2)N2C3CNCC32)n1 ; CC(=O)Nc1ccc2oc(-c3ccnc(C(=O)O)c3)nc2c1 ; CC(=O)Nc1ccc2oc(-c3ccnc(C(=O)N4CC5C(C4)N5C(c4ccccc4)c4nnn(C)n4)c3)nc2c1]

With reference to Example 1, intermediate 53A was synthesized by replacing N-tert-butoxycarbonyl piperazine with tert-butyl 3,6-diazabicyclo[3.1.0]hexane-3-carboxylate as a raw material. Compound 53 (25 mg, 16.14%) was obtained with the same operating method except replacing intermediate 2 with 6-((2-methyl-2H-tetrazol-5-yl)(phenyl)methyl)-3,6-diazabicyclo[3.1.0]hexane (53A).

LC-MS (ESI): m/z=536.2 [M+H]⁺.

### Example 54

### N-(2-(2-(5-((2-methyl-2H-tetrazol-5-yl)(phenyl)methyl)-2,5-diazabicyclo[2.2.2]octane-2-carbonyl)pyridin-4-yl)benzo[d]oxazol-5-yl)acetamide

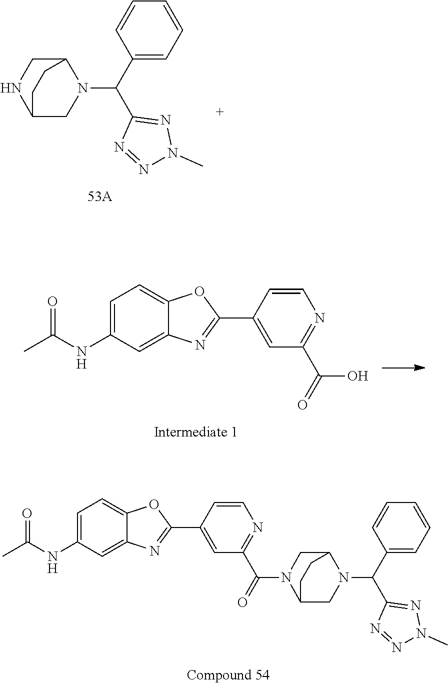 [SMILES: Cn1nnc(C(c2ccccc2)N2CC3CCC2CN3)n1 ; CC(=O)Nc1ccc2oc(-c3ccnc(C(=O)O)c3)nc2c1 ; CC(=O)Nc1ccc2oc(-c3ccnc(C(=O)N4CC5CCC4CN5C(c4ccccc4)c4nnn(C)n4)c3)nc2c1]

With reference to Example 1, intermediate 54A was synthesized by replacing N-tert-butoxycarbonyl piperazine with tert-butyl 2,5-diazabicyclo[2.2.2]hexane-2-carboxylate as a raw material. Compound 54 (25 mg, 16.14%) was obtained with the same operating method except replacing intermediate 2 with 2-((2-methyl-2H-tetrazol-5-yl)(phenyl)methyl)-2,5-diazabicyclo[2.2.2]octane (54A).

¹H NMR (400 MHz, DMSO-d₆) δ 10.16 (s, 1H), 8.84 (s, 1H), 8.45 (s, 1H), 8.25-8.13 (m, 2H), 7.83-7.76 (m, 1H), 7.65-7.50 (m, 3H), 7.40-7.22 (m, 3H), 4.81 (s, 1H), 4.30 (s, 3H), 3.78-3.67 (m, 2H), 3.51-3.49 (m, 2H), 3.42-3.35 (m, 2H), 2.93-2.87 (m, 1H), 2.68-2.62 (m, 1H), 2.09 (s, 3H), 1.93-1.78 (m, 1H), 1.78-1.71 (m, 1H).

LC-MS (ESI): m/z=564.2 [M+H]⁺.

### Example 55

### N-(2-(2-(4-((4-methoxyphenyl)(2-methyl-2H-tetrazol-5-yl)methyl)piperazine-1-carbonyl)pyridin-4-yl)benzo[d]oxazol-5-yl)acetamide

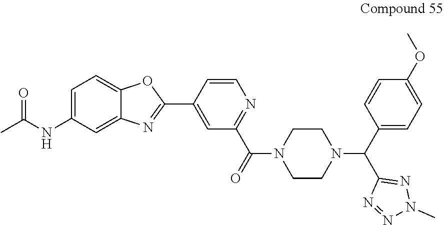 [SMILES: COc1ccc(C(c2nnn(C)n2)N2CCN(C(=O)c3cc(-c4nc5cc(NC(C)=O)ccc5o4)ccn3)CC2)cc1]

P-methoxybenzaldehyde was used as a starting material to obtain compound 55 according to the synthetic step of compound 9.

¹H NMR (400 MHz, CDCl3) δ 8.75-8.74 (m, 1H), 8.35 (s, 1H), 8.10-8.09 (m, 1H), 8.01 (s, 1H), 7.54-7.48 (m, 3H), 7.47-7.44 (s, 2H), 6.91-6.88 (m, 2H), 4.98 (s, 1H), 4.36 (s, 3H), 4.30-4.23 (m, 2H), 3.81 (s, 3H) 3.74-3.50 (m, 2H), 3.35-3.05 (m, 4H), 2.24 (s, 3H).

LC-MS (ESI): m/z=568.2 [M+H]⁺.

### Example 56

### N-(2-(2-(4-((2-(difluoromethyl)-2H-tetrazol-5-yl)(phenyl)methyl)piperazine-1-carbonyl)-3-fluoropyridin-4-yl)benzo[d]oxazol-5-yl)acetamide

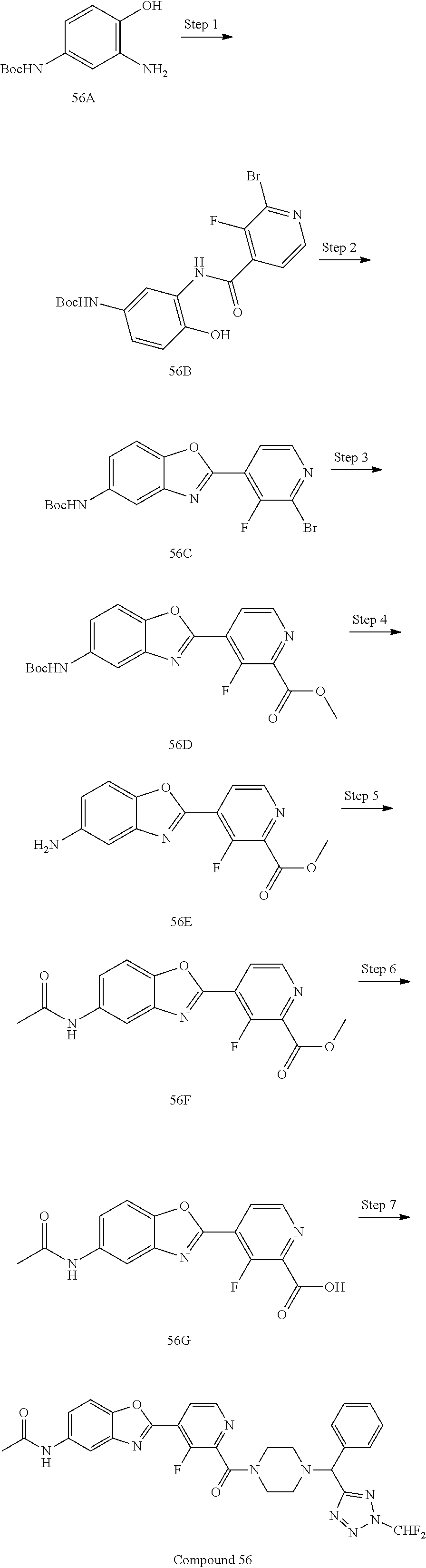

Step 1:

### tert-butyl (3-(2-bromo-3-fluoroisonicotinamido)-4-hydroxyphenyl)carbamate (56B)

Compound 56A (3.9 g, 17.7 mmol), 2-bromo-3-fluoroisonicotinic acid (4 g, 17.7 mmol) and 1-(3-dimethylaminopropyl)-3-ethylcarbodiimide hydrochloride (5 g, 26.5 mmol) were added to pyridine (40 mL), and the mixture was reacted at 85° C. for 5 hours. The reaction solution was cooled to room temperature, then poured into 200 ml of water and filtered. The filtered solid was washed with water and then dried to obtain compound 56B (5 g, 66%).

LC-MS (ESI): m/z=426 [M+H]⁺.

Step 2:

### tert-butyl (2-(2-bromo-3-fluoropyridin-4-yl)benzo[d]oxazol-5-yl)carbamate (56C)

Compound 56B (4.1 g, 9.6 mmol) and triphenylphosphine (3.8 g, 14.4 mmol) were added to tetrahydrofuran (40 mL); diethyl azodicarboxylate (2.5 g, 14.4 mmol) was added dropwise under N2 replacement; and the reaction was stirred at room temperature for 2 hours. After completion of the reaction, water was added, and the resulting solution was extracted with ethyl acetate (50 mL×3). The organic phase was washed with saturated sodium chloride solution, dried over anhydrous sodium sulfate and filtered. The filtrate was concentrated under reduced pressure, and the mixture was slurried with methanol and filtered. The solid filtered was washed with methanol and dried to obtain compound 56C (3.6 g, 92%).

LC-MS (ESI): m/z=408 [M+H]⁺.

Step 3:

### Methyl-4-(5-((tert-butoxycarbonyl)amino)benzo[d]oxazol-2-yl)-3-fluoropicolinate (56D)

Compound 56C (2 g, 4.9 mmol), [1,1′-bis(diphenylphosphino)ferrocene]palladium dichloride dichloromethane complex (400 mg) and triethylamine (1.49 g, 14.7 mmol) were added to a mixed system of methanol (20 mL) and dichloromethane (15 ml), and the mixture was reacted at 80° C. under carbon monoxide (30 MPa) for 3 hours. The reaction solution was cooled to room temperature and filtered. The filtrate was concentrated under reduced pressure, and then the residue was separated and purified by column chromatography (eluent: PE/DCM=1/1) to obtain compound 56D (1.8 g, 95%).

LC-MS (ESI): m/z=388.1 [M+H]⁺.

Step 4:

### Methyl 4-(5-aminobenzo[d]oxazol-2-yl)-3-fluoropicolinate Hydrochloride (56E)

Compound 56D (1.8 g, 4.65 mmol) was added to a solution of hydrochloric acid in dioxane (20 ml), and the mixture was stirred at room temperature for 1 hour. The reaction solution was directly concentrated under reduced pressure to obtain compound 56E (1.5 g, 99%).

Step 5:

### Methyl 4-(5-acetamidobenzo[d]oxazol-2-yl)-3-fluoropicolinate (56F)

Compound 56E (1.5 g, 4.65 mmol) and triethylamine (1.89 g, 18.6 mmol) were added to dichloromethane (30 mL), and acetyl chloride (438 mg, 5.58 mmol) was added dropwise at 0° C. The mixture was reacted at room temperature for 2 hours. The reaction solution was directly concentrated under reduced pressure to obtain a crude product of compound 56F.

LC-MS (ESI): m/z=330.1 [M+H]⁺.

Step 6:

### 4-(5-acetamidobenzo[d]oxazol-2-yl)-3-fluoropicolinic Acid (56G)

Compound 56F and sodium hydroxide (1.12 g, 27.9 mmol) were added to a mixed system of methanol (20 mL) and water (10 ml), and the mixture was reacted at room temperature for 2 hours. The reaction solution was adjusted to pH=1-2 with dilute hydrochloric acid, and a small amount of acetone was added to the reaction solution. The mixture was filtered, and the filtered solid was washed with water to obtain compound 56G (1.2 g).

LC-MS (ESI): m/z=316.1 [M+H]⁺.

Step 7:

### N-(2-(2-(4-((2-(difluoromethyl)-2H-tetrazol-5-yl)(phenyl)methyl)piperazine-1-carbonyl)-3-fluoropyridin-4-yl)benzo[d]oxazol-5-yl)acetamide (Compound 56)

Intermediate 2 (130 mg, 0.44 mmol) and 56G (153 mg, 0.485 mmol) were dissolved in N,N-dimethylformamide (5 mL). HATU (250 mg, 0.66 mmol) and DIPEA (170 mg, 1.32 mmol) were added. After the addition, the mixture was stirred for 1 hour at room temperature, and then the reaction was quenched by adding water (30 mL). The resulting solution was extracted twice with ethyl acetate (30 mL×2). The organic phase was combined, dried over anhydrous sodium sulfate and concentrated under reduced pressure, and then the residue was separated and purified by column chromatography (eluent: MeOH/DCM=1/20) to obtain compound 56 (110 mg, 42%).

¹H NMR (400 MHz, MeOD) δ 8.61 (d, 1H), 8.30-8.02 (m, 3H), 7.69-7.67 (m, 1H), 7.61-7.58 (m, 1H), 7.54-7.52 (m, 2H), 7.39-7.28 (m, 3H), 5.20 (s, 1H), 3.87 (t, 2H), 3.42 (t, 2H), 2.75-2.67 (m, 1H), 2.63-2.53 (m, 2H), 2.47-2.39 (m, 1H), 2.17 (s, 3H).

LC-MS (ESI): m/z=592.2[M+H]⁺.

### Example 57

### N-(2-(2-(4-((2-(difluoromethyl)-2H-tetrazol-5-yl)(phenyl)methyl)piperazine-1-carbonyl)-5-fluoropyridin-4-yl)benzo[d]oxazol-5-yl)acetamide

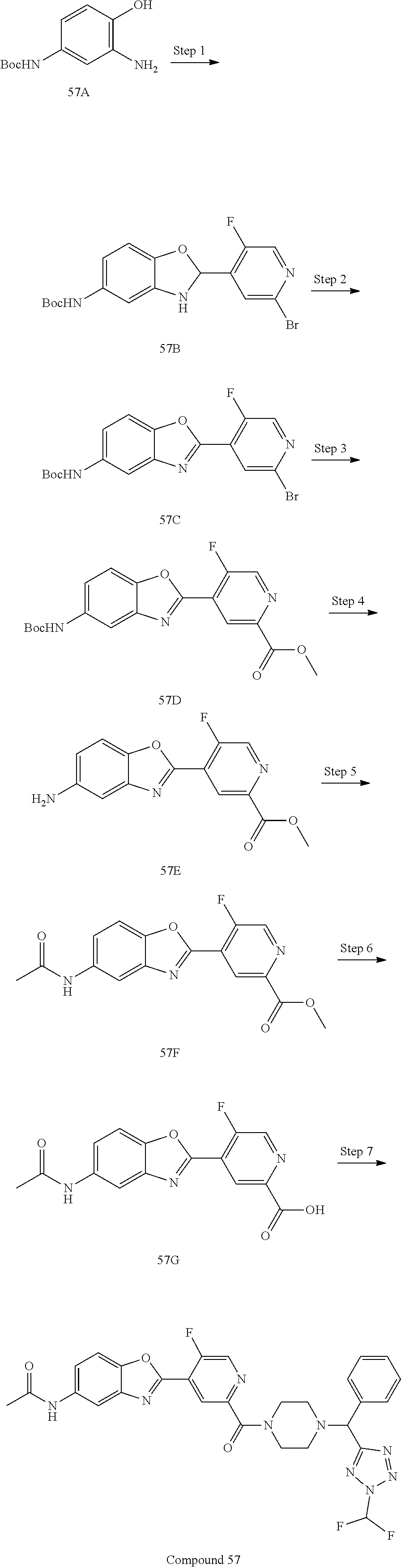

Step 1:

### tert-butyl(2-(2-bromo-5-fluoropyridin-4-yl)-2,3-dihydrobenzo[d]oxazol-5-yl)carbamate (57B)

Compound 57A (3.3 g, 14.7 mmol) and 2-bromo-5-fluoro-4-pyridylaldehyde (3 g, 14.7 mmol) were added to methanol (40 mL), and the mixture was reacted at room temperature for 1 h and directly concentrated under reduced pressure to obtain a crude product of 57B.

Step 2:

### tert-butyl (2-(2-bromo-5-fluoropyridin-4-yl)benzo[d]oxazol-5-yl)carbamate (57C)

Compound 57B from the previous step was added to dichloromethane (50 ml); then 58% activated manganese dioxide (7.67 g, 88 mmol) was added; and the mixture was reacted at room temperature for 1 h. The reaction solution was directly filtered. The filtrate was concentrated under reduced pressure, and then the residue was slurried with methanol and filtered. The filtered solid was washed with methanol and then dried to obtain 57C (4 g).

LC-MS (ESI): m/z=408 [M+H]⁺, 410 [M+H]⁺.

Step 3:

### Methyl 4-(5-((tert-butoxycarbonyl)amino)benzo[d]oxazol-2-yl)-5-fluoropicolinate (57D)

Compound 57C (2 g, 4.9 mmol), [1,1′-bis(diphenylphosphino)ferrocene]palladium dichloride dichloromethane complex (200 mg) and triethylamine (1.48 g, 14.7 mmol) were added to a mixed system of methanol (30 mL) and dichloromethane (15 ml), and the mixture was reacted at 80° C. under carbon monoxide (30 MPa) for 3 hours. The reaction solution was cooled to room temperature and filtered. The filtrate was concentrated under reduced pressure, and then the residue was separated and purified by column chromatography (eluent: PE/DCM=1/1) to obtain compound 57D (1.7 g, 90%).

LC-MS (ESI): m/z=388.1 [M+H]⁺.

Step 4:

### Methyl 4-(5-aminobenzo[d]oxazol-2-yl)-5-fluoropicolinate Hydrochloride (57E)

Compound 57D (1.7 g, 4.39 mmol) was added to a solution of hydrochloric acid in dioxane (30 ml), and the mixture was stirred at room temperature for 1 hour. The reaction solution was directly concentrated under reduced pressure to obtain compound 57E (1.4 g, 97%).

Step 5:

### Methyl 4-(5-acetamidobenzo[d]oxazol-2-yl)-5-fluoropicolinate (57F)

Compound 57E (1.4 g, 4.36 mmol) and triethylamine (1.76 g, 17.4 mmol) were added to dichloromethane (30 mL), and acetyl chloride (410 mg, 5.23 mmol) was added dropwise at 0° C. The mixture was reacted at room temperature for 2 hours. The reaction solution was directly concentrated under reduced pressure to obtain a crude product of compound 57F.

LC-MS (ESI): m/z=330.1 [M+H]⁺.

Step 6:

### 4-(5-acetamidobenzo[d]oxazol-2-yl)-5-fluoropicolinic Acid (57G)

Compound 57F and sodium hydroxide (1.05 g, 26.16 mmol) were added to a mixed system of methanol (20 mL) and water (8 ml), and the mixture was reacted at room temperature for 2 hours. The reaction solution was adjusted to pH=1-2 with dilute hydrochloric acid, and a small amount of acetone was added to the reaction solution. The mixture was filtered, and the filtered solid was washed with water to obtain compound 57G (1.2 g).

LC-MS (ESI): m/z=316.1 [M+H]⁺.

Step 7:

### N-(2-(2-(4-((2-(difluoromethyl)-2H-tetrazol-5-yl)(phenyl)methyl)piperazine-1-carbonyl)-5-fluoropyridin-4-yl)benzo[d]oxazol-5-yl)acetamide (Compound 57)

Intermediate 2 (130 mg, 0.44 mmol) and 57G (153 mg, 0.485 mmol) were dissolved in N,N-dimethylformamide (5 mL). HATU (250 mg, 0.66 mmol) and DIPEA (170 mg, 1.32 mmol) were added. After the addition, the mixture was stirred for 1 hour at room temperature, and then the reaction was quenched by adding water (30 mL). The resulting solution was extracted twice with ethyl acetate (30 mL×2). The organic phase was combined, dried over anhydrous sodium sulfate and concentrated under reduced pressure, and then the residue was separated and purified by column chromatography (eluent: MeOH/DCM=1/20) to obtain compound 57 (110 mg, 42%).

¹H NMR (400 MHz, MeOD) δ 8.70 (d, 1H), 8.39 (d, 1H), 8.32-8.00 (m, 2H), 7.69-7.67 (m, 1H), 7.60-7.58 (m, 1H), 7.56-7.50 (m, 2H), 7.40-7.29 (m, 3H), 5.18 (s, 1H), 3.83 (t, 2H), 3.63 (t, 2H), 2.74-2.66 (m, 1H), 2.64-2.57 (m, 1H), 2.57-2.49 (m, 1H), 2.48-2.39 (m, 1H), 2.16 (s, 3H).

LC-MS (ESI): m/z=592.2[M+H]⁺.

### Example 58

### N-(2-(2-(4-((2-(difluoromethyl)-2H-tetrazol-5-yl)(phenyl)methyl)piperazine-1-carbonyl)-5-methoxypyridin-4-yl)benzo[d]oxazol-5-yl)acetamide

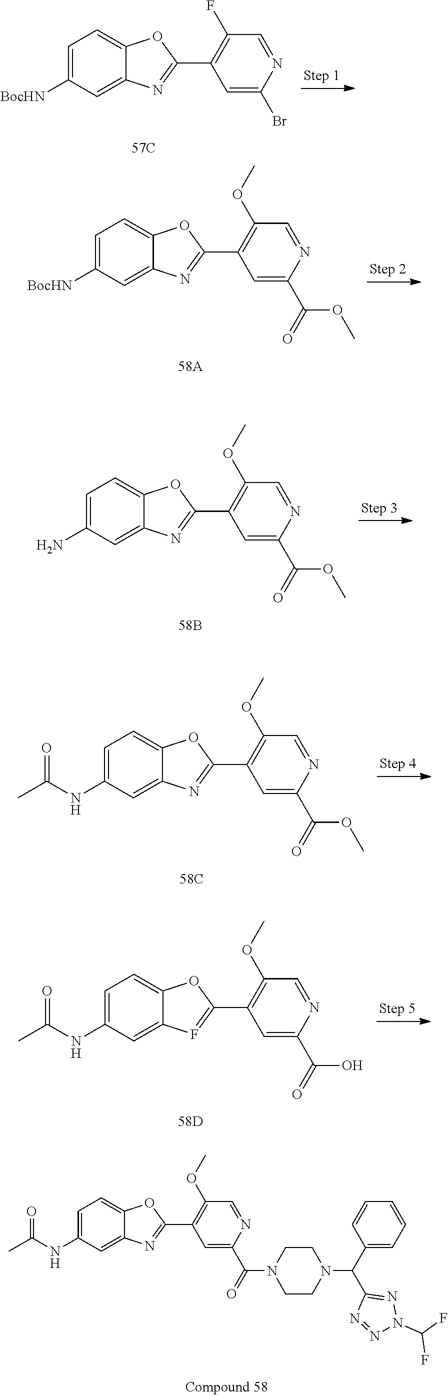

Step 1:

### Methyl 4-(5-((tert-butoxycarbonyl)amino)benzo[d]oxazol-2-yl)-5-methoxypicolinate (58A)

Compound 57C (5 g, 12.3 mmol) and [1,1′-bis(diphenylphosphino)ferrocene]palladium dichloride dichloromethane complex (500 mg) were added to a mixed system of triethylamine (3.71 g, 36.8 mmol) and dichloromethane (30 ml), and the mixture was reacted at 120° C. under carbon monoxide (30 MPa) for 3 hours. The reaction solution was cooled to room temperature and filtered. The filtrate was concentrated under reduced pressure, and then the residue was separated and purified by column chromatography (eluent: PE/DCM=1/1) to obtain compound 58A (1.7 g, 35%).

LC-MS (ESI): m/z=400.1 [M+H]⁺.

Step 2:

### Methyl 4-(5-aminobenzo[d]oxazol-2-yl)-5-methoxypicolinate Hydrochloride (58B)

Compound 58A (1.7 g, 4.26 mmol) was added to a solution of hydrochloric acid in dioxane (30 ml), and the mixture was stirred at room temperature for 1 hour. The reaction solution was directly concentrated under reduced pressure to obtain compound 58B (1.4 g, 99%).

Step 3:

### Methyl 4-(5-acetamidobenzo[d]oxazol-2-yl)-5-methoxypicolinate (58C)

Compound 58B (1.4 g, 4.22 mmol) and triethylamine (1.7 g, 16.88 mmol) were added to dichloromethane (30 mL), and acetyl chloride (400 mg, 5.01 mmol) was added dropwise at 0° C. The mixture was reacted at room temperature for 2 hours. The reaction solution was directly concentrated under reduced pressure to obtain a crude product of compound 58C.

LC-MS (ESI): m/z=342.1 [M+H]⁺.

Step 4:

### 4-(5-acetamidobenzo[d]oxazol-2-yl)-5-methoxypicolinic Acid (58D)

Compound 58C and sodium hydroxide (1.01 g, 25 mmol) were added to a mixed system of methanol (20 mL) and water (8 ml), and the mixture was reacted at room temperature for 2 hours. The reaction solution was adjusted to pH=1-2 with dilute hydrochloric acid, and a small amount of acetone was added to the reaction solution. The mixture was filtered, and the filtered solid was washed with water to obtain compound 58D (1.2 g).

LC-MS (ESI): m/z=328.1 [M+H]⁺.

Step 5:

### N-(2-(2-(4-((2-(difluoromethyl)-2H-tetrazol-5-yl)(phenyl)methyl)piperazine-1-carbonyl)-5-methoxypyridin-4-yl)benzo[d]oxazol-5-yl)acetamide (Compound 58)

Intermediate 2 (130 mg, 0.44 mmol) and 58D (159 mg, 0.484 mmol) were dissolved in N,N-dimethylformamide (5 mL). HATU (250 mg, 0.66 mmol) and DIPEA (170 mg, 1.32 mmol) were added. After the addition, the mixture was stirred for 1 hour at room temperature, and then the reaction was quenched by adding water (30 mL). The resulting solution was extracted twice with ethyl acetate (30 mL×2). The organic phase was combined, dried over anhydrous sodium sulfate and concentrated under reduced pressure, and then the residue was separated and purified by column chromatography (eluent: MeOH/DCM=1/20) to obtain compound 58 (100 mg, 38%).

¹H NMR (400 MHz, MeOD) δ 8.57 (s, 1H), 8.3-8.02 (m, 3H), 7.64-7.61 (m, 1H), 7.58-7.50 (m, 3H), 7.41-7.28 (m, 3H), 5.17 (s, 1H), 4.16 (s, 3H), 3.84-3.82 (m, 2H), 3.71-3.69 (m, 2H), 2.74-2.65 (m, 1H), 2.64-2.57 (m, 1H), 2.56-2.49 (m, 1H), 2.48-2.39 (m, 1H), 2.16 (s, 3H).

LC-MS (ESI): m/z=604.2[M+H]⁺.

### Example 59

### N-(2-(2-(4-((2-(difluoromethyl)-2H-tetrazol-5-yl)(phenyl)methyl)piperazine-1-carbonyl)pyridin-4-yl)-6-fluorobenzo[d]oxazol-5-yl)acetamide

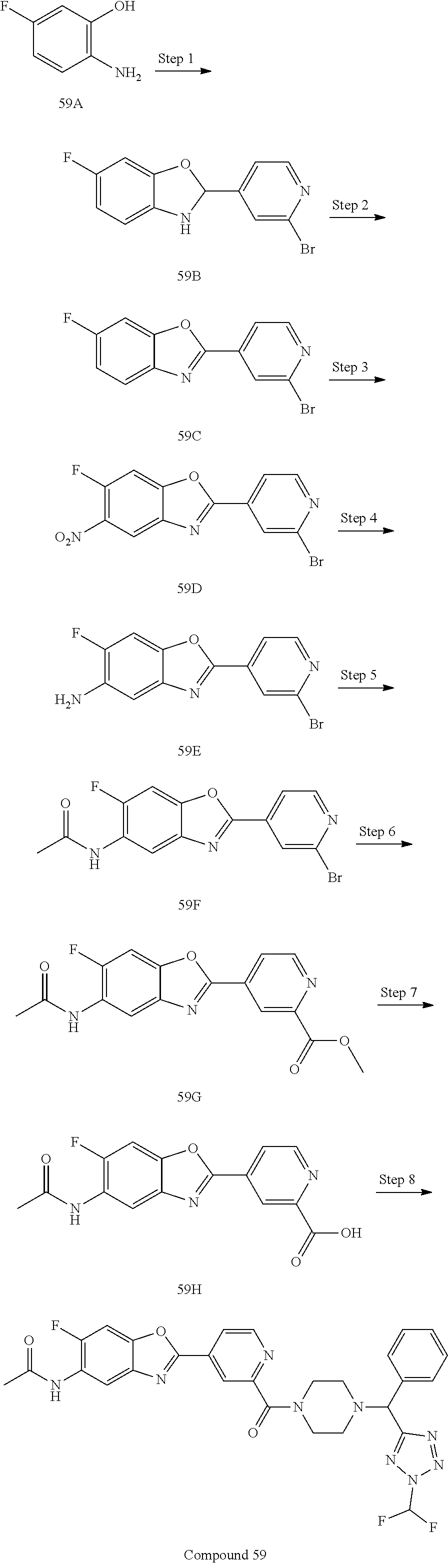

Step 1:

### 2-(2-bromopyridin-4-yl)-6-fluoro-2,3-dihydrobenzo[d]oxazole (59B)

Compound 59A (2.5 g, 19.68 mmol) was dissolved in 50 mL of anhydrous methanol; 2-bromo-4-pyridylaldehyde (4 g, 21.65 mmol) was added; and the mixture was heated to reflux with stirring and reacted for 2 hours. After TLC showed that the reaction was completed, the reaction solution was concentrated to obtain a crude product of 59B.

Step 2:

### 2-(2-bromopyridin-4-yl)-6-fluorobenzo[d]oxazole (59C)

The crude product of 59B obtained in step 1 was dissolved in 50 mL of dichloromethane, and activated manganese dioxide (8.56 g, 98.4 mmol) was added. The reaction was stirred at room temperature for 1 hour. The reaction solution was directly filtered. The filtrate was concentrated under reduced pressure, and then the residue was slurried with methanol and filtered. The filtered solid was washed with methanol and then dried to obtain 59C (2.75 g).

LC-MS (ESI): m/z=292.96 [M+H]⁺.

Step 3:

### 2-(2-bromopyridin-4-yl)-6-fluoro-5-nitrobenzo[d]oxazole (59D)

A mixed solution of concentrated nitric acid (5 mL) and concentrated sulphuric acid (5 mL) was added dropwise to 59C (2.75 g, 9.38 mmol) under an ice bath. After completion of the dropwise addition, the mixture was stirred at room temperature for 3 hours. After TLC showed that the reaction was completed, the reaction solution was added dropwise to saturated aqueous sodium bicarbonate solution until complete neutralization was achieved. The mixture was extracted twice with ethyl acetate. The organic phase was combined, dried and concentrated to obtain 59D (3 g).

LC-MS (ESI): m/z=337.95 [M+H]⁺.

Step 4:

### 2-(2-bromopyridin-4-yl)-6-fluorobenzo[d]oxazol-5-amine (59E)

59D (3 g, 8.87 mmol) was dissolved in 50 mL of anhydrous methanol; 2 mL of saturated ammonium chloride solution was added; and iron powder (1 g, 17.74 mmol) was added with stirring. The mixture was heated to 80° C. and reacted for 4 hours. After TLC showed that the reaction was completed, the reaction solution was concentrated and extracted twice with dichloromethane and water. The organic phase was combined, dried and concentrated to obtain 59E (1.65 g).

LC-MS (ESI): m/z=307.98 [M+H]⁺.

Step 5:

### N-(2-(2-bromopyridin-4-yl)-6-fluorobenzo[d]oxazol-5-yl)acetamide (59F)

59E (1.65 g, 5.35 mmol) was dissolved in 50 mL of dichloromethane; triethylamine (1 g, 10.7 mmol) was added; and acetyl chloride (630 mg, 8.03 mmol) was added dropwise under an ice bath. After completion of the dropwise addition, the mixture was reacted at room temperature for 1 hour. After TLC showed that the reaction was completed, the reaction solution was extracted twice with dichloromethane and water. The organic phase was dried and concentrated to obtain a crude product of 59F.

LC-MS (ESI): m/z=349.99 [M+H]⁺.

Step 6:

### Methyl 4-(5-acetamido-6-fluorobenzo[d]oxazol-2-yl)picolinate (59G)

The crude product of 59F (500 mg) was dissolved in a solution of dichloromethane (25 mL) and methanol (25 mL); and triethylamine (1 mL) and [1,1′-bis(diphenylphosphino)ferrocene]palladium dichloride (224 mg, 0.28 mmol) were added. In an autoclave charged with 150 psi of carbon monoxide gas, the mixture was heated to 80° C. with stirring and reacted for 4 hours. After TLC showed that the reaction was completed, the reaction solution was concentrated. The mixture was slurried with methanol and filtered by suction. The filter cake was dissolved in dichloromethane, and the undissolved solid was filtered by suction. The mother liquor was concentrated to obtain 59G (410 mg).

LC-MS (ESI): m/z=330.08 [M+H]⁺.

Step 7:

### 4-(5-acetamido-6-fluorobenzo[d]oxazol-2-yl)picolinic Acid (59H)

59G (410 mg, 1.25 mmol) was dissolved in anhydrous methanol; lithium hydroxide (150 mg, 6.25 mmol) and 2 mL of water were added; and the mixture was stirred at room temperature for 1 hour. After TLC showed that the reaction was completed, the reaction solution was concentrated to dryness, and the residue was dissolved by adding methanol. The undissolved solid was filtered by suction. The mother liquor was concentrated to obtain a crude product of 59H.

LC-MS (ESI): m/z=315.07 [M+H]⁺.

Step 8:

### N-(2-(2-(4-((2-(difluoromethyl)-2H-tetrazol-5-yl)(phenyl)methyl)piperazine-1-carbonyl)pyridin-4-yl)-6-fluorobenzo[d]oxazol-5-yl)acetamide (Compound 59)

59H (100 mg, 0.32 mmol) was dissolved in 5 mL of N,N-dimethylformamide; HATU (182 mg, 0.48 mmol), N,N-diisopropylethylamine (0.1 mL, 0.64 mmol) and intermediate 6 (188 mg, 0.48 mmol) were added; and the mixture was reacted at room temperature for 2 hours. After TLC showed that the reaction was completed, the reaction solution was extracted with ethyl acetate and water and washed twice with saturated brine. The organic phase was dried and concentrated to obtain a crude product of compound 59, which was subjected to preparative chromatography to obtain compound 59 (75 mg, 40%). Preparation method: instrument: waters 2767 (preparative liquid phase chromatographic instrument); chromatographic column: SunFire@ Prep C18 (19 mm×250 mm); The sample was dissolved in DMF and filtered with a 0.45 m filter to prepare a sample solution. Preparative chromatography conditions: mobile phases A and B: mobile phase A: acetonitrile; mobile phase B: water (5 mM ammonium acetate); gradient elution: the content of mobile phase A rises from 40% to 70%; flow rate: 15 ml/min; elution time: 18 min; retention time: 11.06 min.

¹H NMR (400 MHz, CD₃Cl) δ 8.78 (d, 1H), 8.71 (d, 1H), 8.37 (s, 1H), 8.07-8.09 (m, 1H), 7.63-7.77 (m, 1H), 7.48-7.58 (m, 2H), 7.35-7.42 (m, 5H), 5.23 (s, 1H), 3.96 (s, 2H), 3.77 (s, 2H), 2.53-2.81 (m, 4H), 2.27 (s, 3H)

LC-MS (ESI): m/z=592.2 [M+H]⁺.

### Example 60

### N-(2-(2-(4-((2-(difluoromethyl)-2H-tetrazol-5-yl)(phenyl)methyl)piperazine-1-carbonyl)pyridin-4-yl)-2H-indazol-5-yl)acetamide

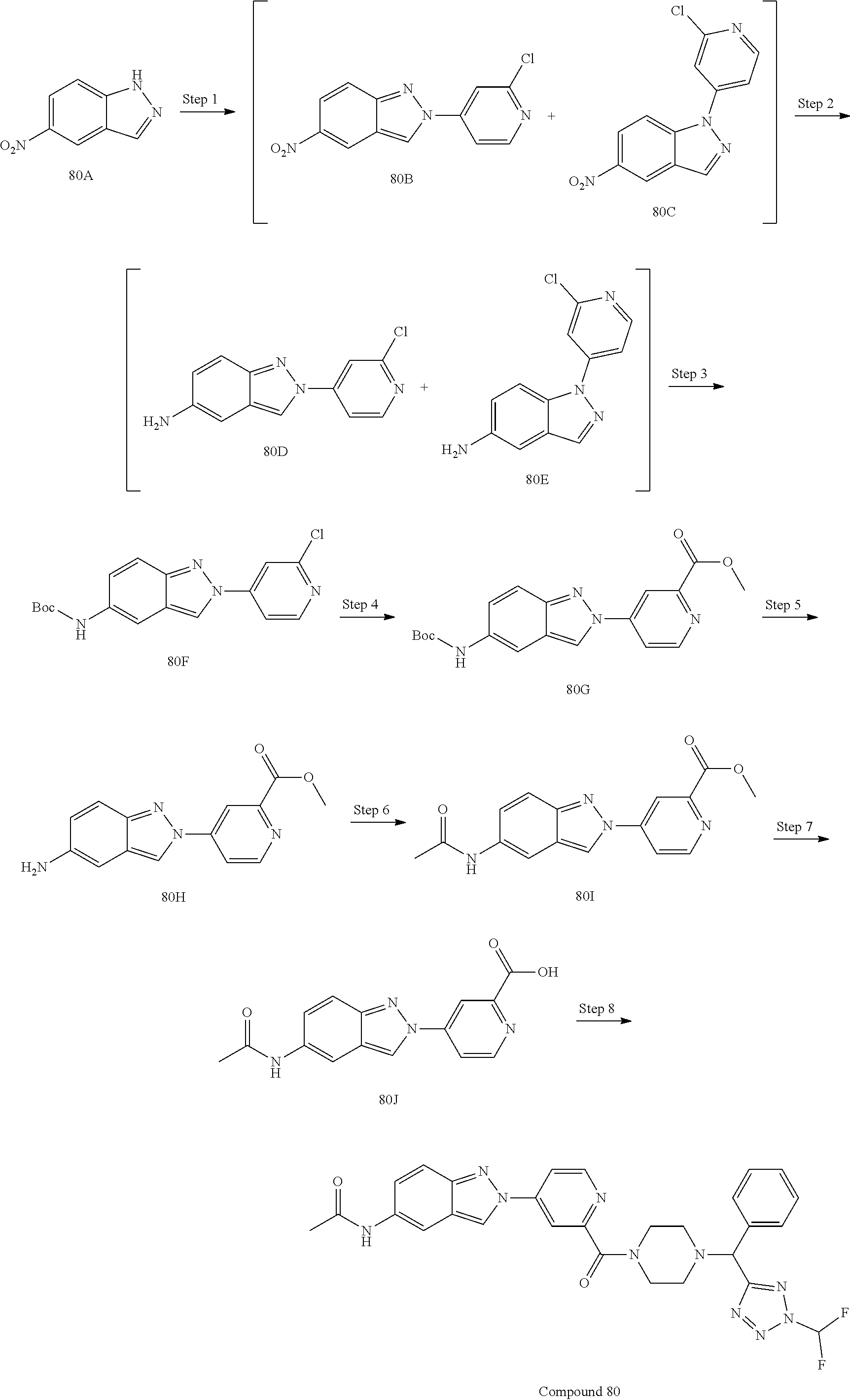

Step 1:

### 2-(2-chloropyridin-4-yl)-5-nitro-2H-indazole (60B)

Under nitrogen protection, sodium hydride (540.0 mg, 13.5 mmol, a content of 600%) was added to DMSO (10 ml), and compound 60A (2.0 g, 12.3 mmol) was slowly added to the reaction solution. The temperature was warmed to 90° C., and then 2-chloro-4 fluoropyridine (1.6 g, 12.3 mmol) was slowly added dropwise. After the addition, the mixture was reacted at 90° C. for 3 hours. The reaction solution was cooled to room temperature, poured into ice water (50 ml) and extracted with ethyl acetate (100 mL×2). The organic layer was combined, washed with water and brine, dried and filtered. The filtrate was concentrated under reduced pressure to obtain a mixture of compound 60B and compound 60C (2.3 g, 62%).

LC-MS (ESI): m/z=275.0 [M+H]⁺.

Step 2:

### 2-(2-chloropyridin-4-yl)-2H-indazol-5-amine (60D)

A mixture of compound 60B and compound 60C (2.3 g, 8.4 mmol) was dissolved in acetic acid (20 ml). Zinc powder (2.7 g, 42.0 mmol) was added at room temperature. After the addition, the mixture was reacted at room temperature for 2 hours. The filtrate was filtered under reduced pressure and concentrated to obtain a mixture of compound 60D and compound 60E (1.8 g, 88%).

LC-MS (ESI): m/z=245.0 [M+H]⁺.

Step 3:

### tert-butyl (2-(2-chloropyridin-4-yl)-2H-indazol-5-yl)carbamate (60F)

A mixture of compound 60D and compound 60E (1.8 g, 7.4 mmol) was dissolved in tetrahydrofuran (20 ml), and triethylamine (2.2 g, 22.2 mmol) and di-tert-butyl dicarbonate (2.4 g, 11.1 mmol) were added. After the addition, the mixture was reacted at room temperature for 16 hours. After completion of the reaction, the reaction solution was concentrated directly, and the residue was separated and purified by column chromatography (eluent: EA/PE=0%-20%) to obtain compound 60F (350 mg, 14%).

LC-MS (ESI): m/z=345.0 [M+H]⁺.

¹H NMR (400 MHz, DMSO-d6) δ 9.42 (s, 1H), 9.19 (s, 1H), 8.56 (d, 1H), 8.21 (d, 1H), 8.12-8.11 (m, 1H), 7.93 (s, 1H), 7.65-7.62 (m, 1H), 7.37-7.34 (m, 1H), 1.50 (s, 9H).

Step 4:

### Methyl 4-(5-((tert-butoxycarbonyl)amino)-2H-indazol-2-yl)picolinate (60G)

Methanol (10 mL), dichloromethane (10 mL), Pd(dppf)Cl₂ (140.0 mg, 0.2 mmol) and triethylamine (404.0 mg, 4.0 mmol) were successively added to compound 60F (350.0 mg, 1.0 mmol); carbon monoxide was introduced; and then the reaction was warmed to 120° C. and stirred for 14 h. The reaction was cooled to room temperature and then filtered. The filtrate was concentrated under reduced pressure, and then the resulting residue was separated and purified by silica gel column chromatography (eluent: EA/PE=10%-30%) to obtain compound 60G (220 mg, 60%).

LC-MS (ESI): m/z=369.2 [M+H]⁺.

Step 5:

### Methyl 4-(5-amino-2H-indazol-2-yl)picolinate (60H)

Compound 60G (220 mg, 0.6 mmol) was dissolved in dichloromethane (5 ml), and a solution of hydrogen chloride in dioxane (3 ml) was added. After the addition, the mixture was stirred at room temperature for 15 hours and concentrated under reduced pressure to obtain compound 60H (160 mg, 99%).

LC-MS (ESI): m/z=269.1 [M+H]⁺.

Step 6:

### Methyl 4-(5-acetamido-2H-indazol-2-yl)picolinate (60I)

Under nitrogen protection, compound 60H (160.0 mg, 0.6 mmol) was dissolved in dichloromethane (5 ml), and triethylamine (180.0 mg, 1.8 mmol) was added, and the mixture was cooled to 0° C.; and acetyl chloride (55.0 mg, 0.7 mmol) was slowly added dropwise. After the addition, the mixture was reacted at 0° C. for 1 hour. The reaction solution was poured into ice water (10 ml) and extracted with dichloromethane (50 mL×2). The organic layer was combined, washed with saturated brine, dried over anhydrous sodium sulfate and filtered. The residue was dried and concentrated to obtain a crude product of compound 60I (150 mg, 80%), which was directly used in the next step.

LC-MS (ESI): m/z=311.1[M+H]⁺.

¹H NMR (400 MHz, DMSO-d6) δ 10.1 (s, 1H), 9.32 (s, 1H), 8.82 (d, 1H), 8.70 (d, 1H), 8.31 (d, 1H), 8.24 (d, 1H), 7.69 (d, 1H), 7.40-7.31 (m, 1H), 3.95 (s, 3H), 2.10 (s, 3H).

Step 7:

### 4-(5-acetamido-2H-indazol-2-yl)picolinic Acid (60J)

Compound 60I (150 mg, 0.5 mmol) was dissolved in methanol (5 ml), and sodium hydroxide (32 mg, 0.8 mmol, 5 ml) aqueous solution was added. The mixture was stirred at room temperature for 3 hours, concentrated under reduced pressure to remove most of methanol, adjusted to pH=2-3 by dropwise adding 1 N hydrochloric acid and filtered. The filter cake was washed with water (3 mL) and dried to obtain compound 60J (110 mg, 75%).

LC-MS (ESI): m/z=297.1[M+H]⁺.

Step 8:

### N-(2-(2-(4-((2-(difluoromethyl)-2H-tetrazol-5-yl)(phenyl)methyl)piperazine-1-carbonyl)pyridin-4-yl)-2H-indazol-5-yl)acetamide (Compound 60)

Compound 60J (110.0 mg, 0.4 mmol) was dissolved in N,N-dimethylformamide (5 ml), and the mixture was cooled to 0° C.; HATU (190.0 mg, 0.5 mmol) and DIPEA (206.0 mg, 1.6 mmol) were added; and then intermediate 6 (150.0 mg, 0.5 mmol) was added. After the addition, the mixture was stirred for 1 hour while the temperature was controlled at 0° C., and then the reaction was quenched by adding ice water (5 ml). The resulting solution was extracted twice with dichloromethane (20 mL×2). The organic phase was combined, dried over anhydrous sodium sulfate and filtered. The filtrate was concentrated under reduced pressure, and then the residue was separated and purified by column chromatography (eluent: EA/DCM=30%-60%) to obtain compound 60 (73 mg, 34%).

LC-MS (ESI): m/z=573.2[M+H]⁺.

¹H NMR (400 MHz, CDCl₃) δ 8.64 (d, 1H), 8.38 (s, 1H), 8.15 (s, 1H), 8.10 (s, 1H), 7.93-7.92 (m, 1H), 7.65 (s, 1H), 7.63 (s, 1H), 7.58-7.56 (m, 2H), 7.40-7.34 (m, 4H), 7.11-7.08 (m, 1H), 5.24 (s, 1H), 3.95-3.81 (m, 4H), 2.83-2.55 (m, 4H), 2.20 (s, 3H).

### Example 61

### N-(2-(2-(4-((2-(difluoromethyl)-2H-tetrazol-5-yl)(phenyl)methyl)piperazine-1-carbonyl)pyridin-4-yl)-7-fluorobenzo[d]oxazol-5-yl)acetamide

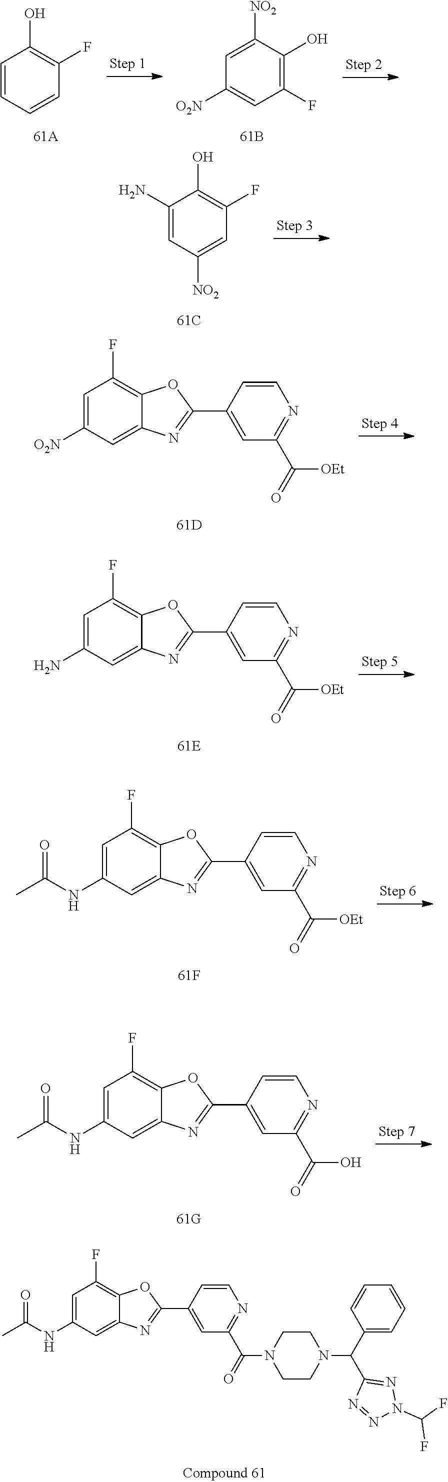

Step 1:

### 2-fluoro-4,6-dinitrophenol (61B)

Compound 61A (5.0 g, 44.6 mmol) was dissolved in dichloromethane (50 ml), and the mixture was cooled to 0° C.; and concentrated nitric acid (10.7 g, 107 mmol, a content of 65%) was slowly added dropwise. After the dropwise addition was completed, the mixture was reacted for 2 hours while the temperature was controlled at 0° C. The reaction solution was poured into 50 ml of ice water and extracted with dichloromethane (100 mL×2). The organic layer was combined, washed with saturated brine, dried over anhydrous sodium sulfate and filtered. The filtrate was concentrated and then crystallized with petroleum ether to obtain compound 61B (7.2 g, 80%).

LC-MS (ESI): m/z=203.1 [M+H]⁺.

Step 2:

### 2-amino-6-fluoro-4-nitrophenol (61C)

Compound 61B (7.2 g, 35.0 mmol) was dissolved in anhydrous ethanol (50 ml), and stannous chloride (11.8 g, 52.5 mmol) was added. After the addition, the mixture was warmed to 70° C. and reacted for 4 hours. The reaction was cooled to room temperature and adjusted to pH=5-6 with 10% sodium hydroxide solution. The reaction solution was filtered. The filtrate was extracted with ethyl acetate (100 mL×2). The organic layer was combined, washed with saturated brine, dried over anhydrous sodium sulfate and filtered. The filtrate was concentrated under reduced pressure. The residue was crystallized with petroleum ether to obtain compound 61C (4.8 g, 79%).

LC-MS (ESI): m/z=173.1 [M+H]⁺.

Step 3:

### Ethyl 4-(7-fluoro-5-nitrobenzo[d]oxazol-2-yl)picolinate (61D)

Compound 61C (1.2 g, 6.9 mmol) was dissolved in ethanol (20 ml), and ethyl 4-formylpicolinate (1.5 g, 8.3 mmol) was added. After the addition, the mixture was warmed to 70° C., reacted for 16 hours and concentrated under reduced pressure to remove methanol, and then dichloromethane (50 ml) and DDQ (2.4 g, 10.4 mmol) were added. After the addition, the mixture was reacted at room temperature for 2 hours. The reaction solution was poured into saturated aqueous sodium bicarbonate solution (50 ml) and extracted with dichloromethane (100 mL×2). The organic layer was combined, washed with saturated brine, dried over anhydrous sodium sulfate and filtered. The filtrate was concentrated under reduced pressure. The residue was purified by silica gel column chromatography (eluent: EA/PE=10%-30%) to obtain compound 61D (0.9 g, 39%).

LC-MS (ESI): m/z=332.2 [M+H]⁺.

¹H NMR (400 MHz, DMSO-d6) δ 9.04 (d, 1H), 8.73 (d, 1H), 8.67 (d, 1H), 8.49-8.46 (m, 1H), 8.41 (d, 1H), 4.47-4.41 (m, 2H), 1.41-1.37 (m, 3H).

Step 4:

### Ethyl 4-(5-amino-7-fluorobenzo[d]oxazol-2-yl)picolinate (61E)

Compound 61D (0.9 g, 2.7 mmol) was dissolved in methanol (20 ml), and 10% palladium on carbon (0.2 g) was added; hydrogen was introduced, and the mixture was reacted at room temperature for 3 hours. The reaction solution was filtered through celite. The filtrate was concentrated to obtain compound 61E (0.7 g, 85%).

LC-MS (ESI): m/z=302.2 [M+H]⁺.

Step 5:

### Ethyl 4-(5-acetamido-7-fluorobenzo[d]oxazol-2-yl)picolinate (61F)

Under nitrogen protection, compound 61E (0.7 g, 2.3 mmol) was dissolved in dichloromethane (10 ml), and triethylamine (0.5 g, 4.8 mmol) was added, and the mixture was cooled to 0° C.; and acetyl chloride (214.0 mg, 2.8 mmol) was slowly added dropwise. After the addition, the mixture was reacted at 0° C. for 1 hour. The reaction solution was poured into ice water (50 ml) and extracted with dichloromethane (50 mL×2). The organic layer was combined, washed with saturated brine, dried over anhydrous sodium sulfate and filtered. The filtrate was dried and concentrated under reduced pressure to obtain a crude product of compound 61F (620 mg, 78%), which was directly used in the next step.

LC-MS (ESI): m/z=344.3[M+H]⁺.

Step 6:

### 4-(5-acetamido-7-fluorobenzo[d]oxazol-2-yl)picolinic Acid (61G)

Compound 61F (620 mg, 1.8 mmol) was dissolved in methanol (5 ml), and sodium hydroxide (108 mg, 2.7 mmol, 5 ml) aqueous solution was added. The mixture was stirred at room temperature for 3 hours, concentrated under reduced pressure to remove most of methanol, adjusted to pH=2-3 by dropwise adding 1 N hydrochloric acid and filtered. The filter cake was washed with water (3 mL) and dried to obtain compound 61G (350 mg, 62%).

LC-MS (ESI): m/z=316.2[M+H]⁺.

¹H NMR (400 MHz, DMSO-d6) δ 10.41 (s, 1H), 8.97 (d, 1H), 8.65 (d, 1H), 8.32-8.30 (m, 1H), 7.96 (d, 1H), 7.73-7.70 (m, 1H), 2.10 (s, 3H).

Step 7:

### N-(2-(2-(4-((2-(difluoromethyl)-2H-tetrazol-5-yl)(phenyl)methyl)piperazine-1-carbonyl)pyridin-4-yl)-7-fluorobenzo[d]oxazol-5-yl)acetamide (Compound 61)

Compound 61G (200.0 mg, 0.63 mmol) was dissolved in N,N-dimethylformamide (5 ml), and the mixture was cooled to 0° C.; HATU (287.0 mg, 0.75 mmol) and DIPEA (325.0 mg, 2.5 mmol) were added; and then intermediate 6 (204.0 mg, 0.69 mmol) was added. After the addition, the mixture was stirred for 1 hour while the temperature was controlled at 0° C., and then the reaction was quenched by adding ice water (5 ml). The resulting solution was extracted twice with dichloromethane (20 mL×2). The organic phase was combined, dried over anhydrous sodium sulfate and filtered. The filtrate was concentrated under reduced pressure, and then the residue was separated and purified by column chromatography (eluent: EA/DCM=30%-60%) to obtain compound 61 (130 mg, 35%).

LC-MS (ESI): m/z=592.2[M+H]⁺.

¹H NMR (400 MHz, CDCl₃) δ 8.72 (d, 1H), 8.26 (s, 1H), 8.04 (d, 1H), 8.00 (s, 1H), 7.62 (s, 1H), 7.60 (s, 1H), 7.53-7.46 (m, 3H), 7.39-7.30 (m, 3H), 5.13 (s, 1H), 3.92-3.89 (m, 2H), 3.67-3.64 (m, 2H), 2.75-2.41 (m, 4H), 2.18 (s, 3H).

### Example 62

### N-(2-(4-(4-((2-(difluoromethyl)-2H-tetrazol-5-yl)(phenyl)methyl)piperazine-1-carbonyl)pyridin-2-yl)benzo[d]oxazol-5-yl)acetamide

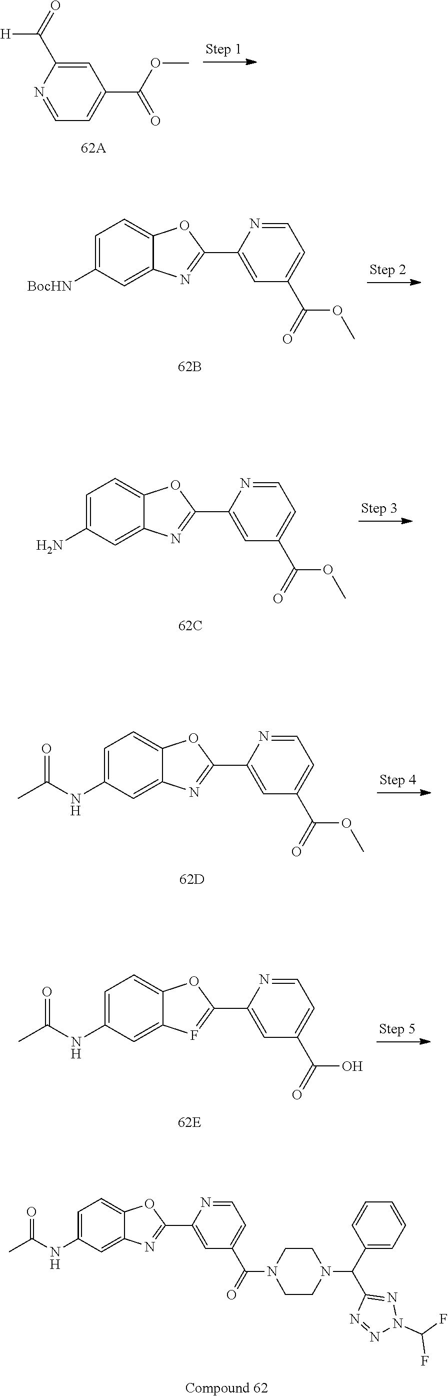

Step 1:

### Methyl 2-(5-((tert-butoxycarbonyl)amino)benzo[d]oxazol-2-yl)picolinate (62B)

Compound tert-butyl(3-amino-4-hydroxyphenyl)carbamate (224 mg, 1 mmol) was dissolved in methanol (5 mL); 62A (165 mg, 1 mmol) was added; and the mixture was warmed to 70° C. and stirred for 15 h. The reaction was cooled to room temperature and concentrated under reduced pressure to remove methanol, and then dichloromethane (10 mL) and MnO2 (435 mg, 5 mmol) were successively added to the residue. The mixture was stirred for 2 h, and saturated aqueous sodium carbonate solution (100 mL) was added. The resulting solution was stirred for 10 min and filtered. The filtrate was extracted with dichloromethane (20 mL×2). The combined organic phase was washed with water (10 mL), dried over anhydrous sodium sulfate and concentrated under reduced pressure, and then the residue was separated and purified by column chromatography (PE:EA=4:1) to obtain 62B (350 mg, 95%).

LC-MS (ESI): m/z=370.3 [M+H]⁺.

Step 2:

### Methyl 2-(5-amino)benzo[d]oxazol-2-yl)picolinate (62C)

Dichloromethane (5 mL) and a solution of hydrochloric acid (4 M) in dioxane (5 ml) were added to compound 62B (350 mg, 0.95 mmol). The mixture was reacted for 2 h and then subjected to rotary evaporation to obtain 62C (250 mg, 97.8%).

LC-MS (ESI): m/z=270.2 [M+H]⁺.

Step 3:

### 2-(5-acetamidobenzo[d]oxazol-2-yl) picolinate (62D)

Dichloromethane (5 mL) and Et3N (188 mg, 1.84 mmol) were added to compound 62C (250 mg, 0.92 mmol). The mixture was stirred at room temperature for 10 min, and acetyl chloride (86.7 mg, 1.1 mmol) was added. The resulting mixture was stirred at room temperature for 1 h, quenched by adding water (10 ml) and extracted with dichloromethane (20 mL×2). The combined organic phase was washed with water (10 mL), dried over anhydrous sodium sulfate and concentrated under reduced pressure, and then the residue was separated and purified by silica gel column chromatography (PE:EA=2:1) to obtain 62D (280 mg, 97.5%).

LC-MS (ESI): m/z=312.3 [M−H]⁻.

Step 4:

### 2-(5-acetamidobenzo[d]oxazol-2-yl)picolinic Acid (62E)

Anhydrous methanol (5 mL) and NaOH (107.7 mg, 2.7 mmol, 2 mL) aqueous solution were successively added to compound 62D (1.4 g, 4.5 mmol). The mixture was stirred at room temperature for 5 h, concentrated under reduced pressure to remove most of methanol, adjusted to pH=2-3 by dropwise adding 1 N hydrochloric acid and filtered. The filter cake was washed with water (3 mL) and dried to obtain 62E.

LC-MS (ESI): m/z=298.2 [M−H]⁻.

Step 5:

### N-(2-(4-(4-((2-(difluoromethyl)-2H-tetrazol-5-yl)(phenyl)methyl)piperazine-1-carbonyl)pyridin-2-yl)benzo[d]oxazol-5-yl)acetamide

62E (50 mg, 0.17 mmol) was dissolved in N,N-dimethylformamide (1 mL). HATU (76 mg, 0.2 mmol) and DIPEA (25.8 mg, 0.2 mmol) were added, and then intermediate 6 (50 mg, 0.17 mmol) was added. After the addition, the mixture was stirred for 1 hour at room temperature, and then the reaction was quenched by adding water (5 mL). The resulting solution was extracted twice with dichloromethane (20 mL×2). The organic phase was combined, dried over anhydrous sodium sulfate and concentrated under reduced pressure. The residue was separated by column chromatography (DCM:CH₃OH=20:1) to obtain compound 62 (36.3 mg, 37%).

¹H NMR (400 MHz, CDCl₃) δ 8.96 (d, J=5.1 Hz, 1H), 8.18 (d, J=5.1 Hz, 1H), 8.04 (s, 1H), 7.63 (s, 1H), 7.58-7.48 (m, 5H), 7.41-7.30 (m, 4H), 5.13 (s, 1H), 3.86 (s, 2H), 3.45 (s, 2H), 2.69 (s, 1H), 2.54 (s, 2H), 2.38 (s, 1H), 2.20 (s, 3H).

LC-MS (ESI): m/z=574.5 [M+H]⁺.

### Example 63

### N-(2-(6-(4-((2-(difluoromethyl)-2H-tetrazol-5-yl)(phenyl)methyl)piperazine-1-carbonyl)pyridin-2-yl)benzo[d]oxazol-5-yl)acetamide

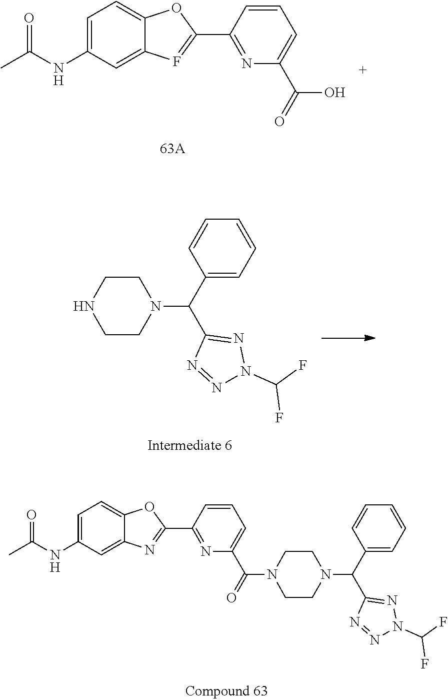

Methyl 6-formylpicolinate was used as a starting material to obtain 63A according to the synthetic step of intermediate 62, and compound 63 was synthesized by using 63A as a starting material with reference to the synthesis of compound 62.

¹H NMR (400 MHz, CDCl₃) δ 8.37-8.34 (m, 1H), 8.00-7.95 (m, 2H), 7.78-7.75 (m, 1H), 7.61 (s, 1H), 7.59-7.48 (m, 4H), 7.38-7.29 (m, 4H), 5.13 (s, 1H), 3.93-3.84 (m, 2H), 3.75-3.73 (m, 2H), 2.75-2.70 (m, 1H), 2.66-2.61 (m, 1H), 2.56-2.51 (m, 1H), 2.47-2.42 (m, 1H), 2.23 (s, 3H).

LC-MS (ESI): m/z=574.5 [M+H]⁺.

### Example 64

### N-(2-(2-(4-((2-(difluoromethyl)-2H-tetrazol-5-yl)(phenyl)methyl)piperazine-1-carbonyl)pyrimidin-4-yl)benzo[d]oxazol-5-yl)acetamide

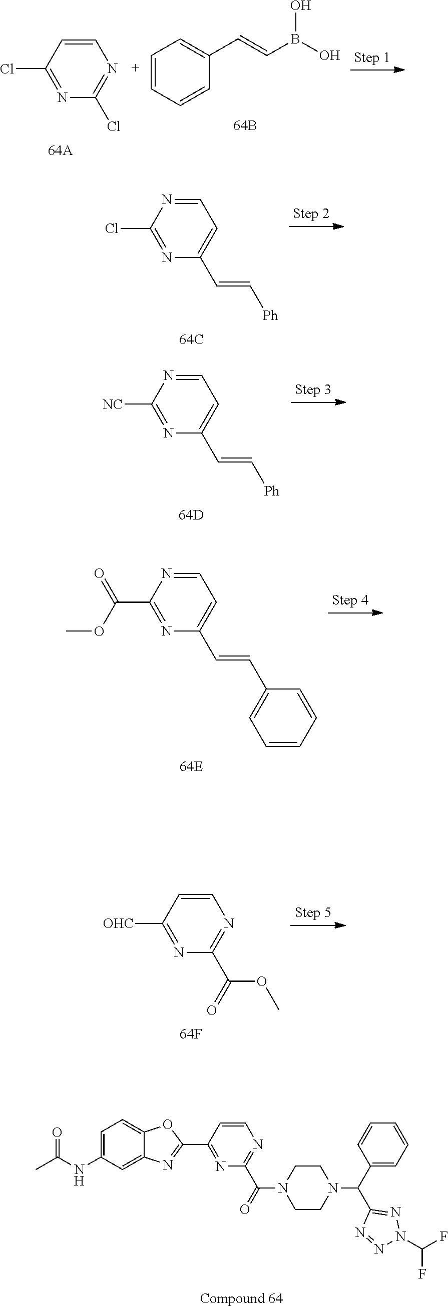

Step 1:

### (E)-2-chloro-4-styrylpyrimidine (64C)

2,4-dichloropyrimidine (64A) (7.5 g, 50 mmol), (E)-phenylethenylboronic acid (64B) (8.88 g, 60 mmol), Pd(PPh₃)₂Cl₂ (3.5 g, 5 mmol) and K₃PO₄ (21.2 g, 100 mmol) were mixed in 1,4-dioxane (100 mL), and the mixture was warmed to 90° C. and reacted for 8 h under nitrogen atmosphere. After the reaction was completed, the reaction solution was cooled to room temperature and diluted with ethyl acetate (100 mL). Saturated aqueous NaHCO₃ solution (200 ml) was added for liquid separation, and the aqueous phase was extracted with ethyl acetate (150 mL×3). The organic phase was combined, washed with saturated brine, dried over anhydrous sodium sulfate and concentrated, and then the residue was separated by silica gel column chromatography (eluent: EA/PE=5%-10%) to obtain target compound 64C (6.7 g, 62%).

¹H NMR (400 MHz, DMSO-d6) δ 8.72 (d, 1H), 7.95 (d, 1H), 7.79-7.73 (m, 2H), 7.66 (d, 1H), 7.50-7.38 (m, 3H), 7.33 (d, 1H).

Step 2:

### (E)-4-styrylpyrimidine-2-carbonitrile (64D)

(E)-2-chloro-4-styrylpyrimidine (64C) (6.7 g, 31 mmol), Zn(CN)₂ (5.3 g, 45 mmol), and Zn (0.39 g, 6 mmol) were mixed in DMA (55 mL). Pd₂(dba)₃ (2.7 g, 3 mmol) and dppf (3.3 g, 6 mmol) were added under nitrogen atmosphere, and the mixture was warmed to 100° C. and reacted for 3 h under nitrogen atmosphere. The reaction solution was cooled to room temperature and diluted with ethyl acetate (100 mL). Saturated aqueous NaHCO₃ solution (150 ml) was added for liquid separation, and the aqueous phase was extracted with ethyl acetate (150 mL×3). The organic phase was combined, washed with saturated brine, dried over anhydrous sodium sulfate and concentrated, and then the residue was separated by silica gel column chromatography (eluent: EA/PE=6%-12%) to obtain target compound 64D (5.1 g, 79%).

LC-MS (ESI): m/z=208.1 [M+H]⁺.

Step 3:

### (E)-methyl 4-styrylpyrimidine-2-carboxylate (64E)

(E)-4-styrylpyrimidine-2-carbonitrile (64D) (5.1 g, 25 mmol) was dissolved in methanol (30 mL) and concentrated hydrochloric acid (30 mL), and the mixture was warmed to 100° C. and reacted for 2 h. After the reaction was completed, the reaction solution was cooled to room temperature. The reaction was quenched by saturated sodium bicarbonate and adjusted to pH 4 with dilute hydrochloric acid. The reaction solution was extracted with ethyl acetate (200 mL×3). The organic phase was combined, washed with saturated brine, dried over anhydrous sodium sulfate and concentrated, and then the residue was separated by silica gel column chromatography (eluent: EA/PE=6%-15%) to obtain target compound 64E (0.78 g, 13%).

LC-MS (ESI): m/z=241.1 [M+H]⁺.

Step 4:

### Methyl 4-formylpyrimidine-2-carboxylate (64F)

(E)-methyl 4-styrylpyrimidine-2-carboxylate (64E) (0.78 g) was dissolved in tetrahydrofuran (15 mL) and water (5 mL), and the system was cooled down to 0° C. Sodium periodate (2.14 g, 10 mmol) and potassium osmium tetroxide (0.1 g) were successively added, and the reaction was stirred for 3 h at 0° C. under an ice bath. The reaction was quenched with saturated Na₂S₂O₃ (100 mL) and extracted with EA (100 mL×3). The organic phase was combined, dried over anhydrous sodium sulfate and filtered. The filtrate was concentrated under reduced pressure. The residue was separated by silica gel column chromatography (eluent: EA/PE=50%-100%) to obtain compound 64F (0.23 g, 43%).

LC-MS (ESI): m/z=167.1 [M+H]⁺, 185.1 [M+H₂O]⁺.

¹H NMR (400 MHz, CDCl₃) δ 10.18 (s, 1H), 9.22 (d, J=4.9, 1H), 8.00 (d, J=4.9 Hz, 1H), 4.13 (s, 3H).

Step 5:

### N-(2-(2-(4-((2-(difluoromethyl)-2H-tetrazol-5-yl)(phenyl)methyl)piperazine-1-carbonyl)pyrimidin-4-yl)benzo[d]oxazol-5-yl)acetamide (Compound 64)

64F was used as a starting material to obtain compound 64 according to the synthetic step of compound 62.

LC-MS (ESI): m/z=575.3 [M+H]⁺.

¹H NMR (400 MHz, CDCl₃) δ 8.96 (d, J=5.1 Hz, 1H), 8.18 (d, J=5.1 Hz, 1H), 8.04 (s, 1H), 7.78 (s, 1H), 7.66-7.46 (m, 5H), 7.43-7.30 (m, 3H), 5.26 (s, 1H), 3.97 (s, 2H), 3.53 (s, 2H), 2.86 (s, 1H), 2.75 (s, 1H), 2.67 (s, 1H), 2.54 (s, 1H), 2.20 (s, 3H).

### Example 66

### N-(2-(2-(4-((4-cyanophenyl)(2-methyl-2H-tetrazol-5-yl)methyl)piperazine-1-carbonyl)pyridin-4-yl)benzo[d]oxazol-5-yl)acetamide

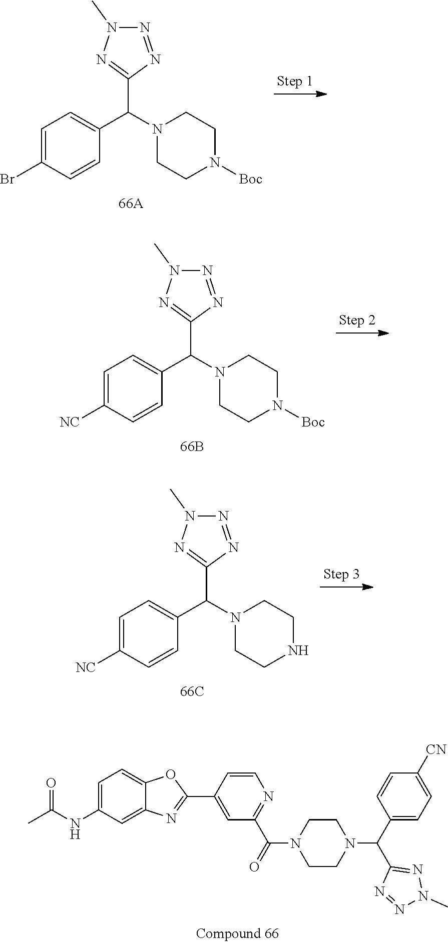 [SMILES: Cn1nnc(C(c2ccc(Br)cc2)N2CCN(C(=O)OC(C)(C)C)CC2)n1 ; Cn1nnc(C(c2ccc(C#N)cc2)N2CCN(C(=O)OC(C)(C)C)CC2)n1 ; Cn1nnc(C(c2ccc(C#N)cc2)N2CCNCC2)n1 ; CC(=O)Nc1ccc2oc(-c3ccnc(C(=O)N4CCN(C(c5ccc(C#N)cc5)c5nnn(C)n5)CC4)c3)nc2c1]

Tert-butyl 4-((4-bromobenzyl)(2-methyl-2H-tetrazol-5-yl)methyl)piperazine-1-carbonate (compound 66A) was synthesized with reference to the same operating method as that of intermediate 2. Then compound 66 was synthesized according to the following synthetic method using compound 66A as a raw material.

Step 1:

### tert-butyl-4-((4-cyanophenyl)(2-methyl-2H-tetrazol-5-yl)methyl)piperazine-1-carboxylate (66B)

Under nitrogen protection, compound 66A (0.67 g, 1.53 mmol), Pd(dba)₂ (140 mg, 0.243 mmol), dppf (1,1′-bis(diphenylphosphino)ferrocene) (170 mg, 0.31 mmol), Zn(CN)₂ (216 mg, 1.83 mmol), zinc powder (20 mg, 0.31 mmol) and DMA (10 mL) were successively added to a single-necked flask, and the reaction was stirred at 120° C. for 24 hours. The reaction was cooled to room temperature and filtered. Water (100 mL) was added to the filtrate, and the residue was extracted with ethyl acetate (100 mL×3). The combined organic phase was dried over anhydrous sodium sulfate and concentrated under reduced pressure, and then the residue was separated and purified by silica gel column chromatography (eluent: EA/PE=1/4) to obtain compound 66B (450 mg, 76.6%).

LC-MS (ESI): m/z=384.3[M+H]⁺.

Step 2:

### 1-((2-methyl-2H-tetrazol-5-yl)(phenyl)methyl)piperazine (66C)

Compound 66B (120 mg, 0.47 mmol), methanol (5 mL) and trifluoroacetic acid (2 mL) were successively added to a single-necked flask. The reaction was stirred at room temperature for 2 h and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure, and then the residue was separated and purified by silica gel column chromatography (eluent: DCM/MeOH=1/60) to obtain compound 66C (80 mg, 98%).

LC-MS (ESI): m/z=284.2[M+H]⁺.

Step 3:

### N-(2-(2-(4-((4-cyanophenyl)(2-methyl-2H-tetrazol-5-yl)methyl)piperazine-1-carbonyl)pyridin-4-yl)benzo[d]oxazol-5-yl)acetamide (Compound 66)

Under nitrogen protection, intermediate 1 (115 mg, 0.38 mmol), dichloromethane (10 mL), DIPEA (80 mg, 0.57 mmol), HATU (173 mg, 0.45 mmol), and 66C (100 mg, 0.38 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (100 mL×2). The combined organic phase was dried over anhydrous sodium sulfate and concentrated under reduced pressure to obtain a crude product from the residue, and the crude product was subjected to preparative high-performance liquid chromatography to obtain compound 66 (35 mg, 16.07%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.45 min.

¹H NMR (400 MHz, CDCl₃) δ 8.71 (d, J=5.1 Hz, 1H), 8.32-8.23 (m, 1H), 8.05 (dd, J=5.1, 1.6 Hz, 1H), 7.96 (s, 1H), 7.76 (s, 1H), 7.71-7.60 (m, 4H), 7.47 (s, 2H), 5.11 (s, 1H), 4.37 (s, 3H), 3.88 (s, 2H), 3.65 (t, J=5.1 Hz, 2H), 2.69-2.36 (m, 4H), 2.20 (s, 3H).

LC-MS (ESI): m/z=563.3 [M+H]⁺.

### Example 67

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclobutanecarboxamide

### Example 68

### (S/R)—N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclobutanecarboxamide

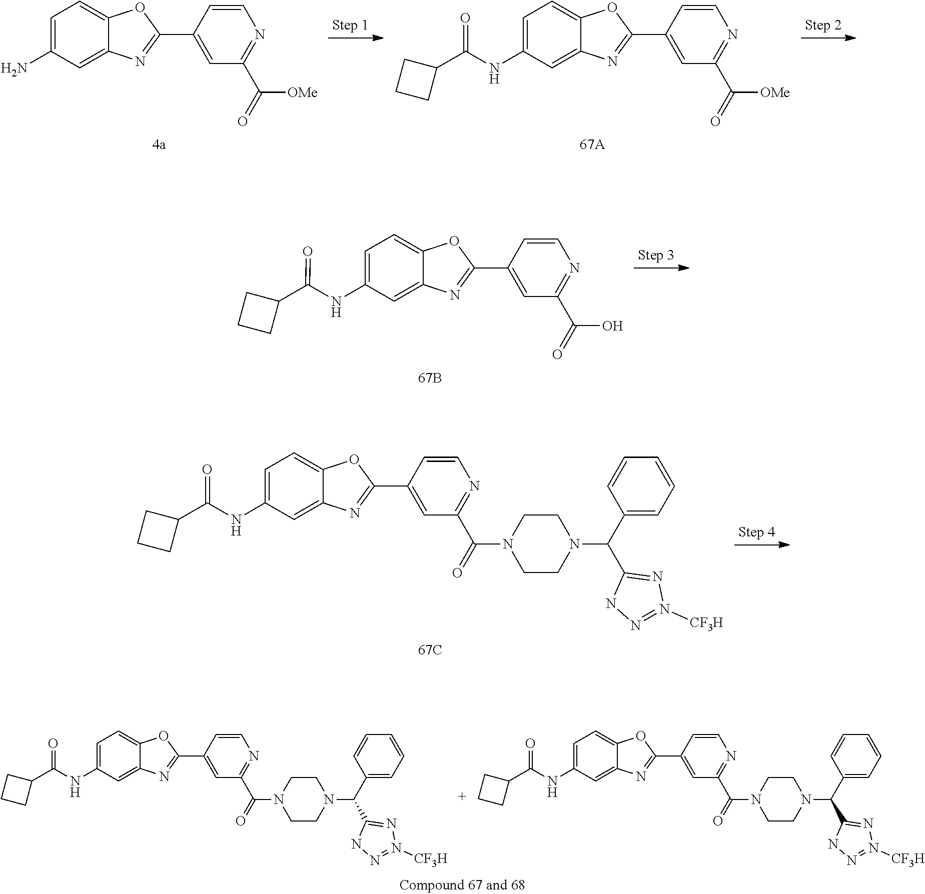

Step 2:

### 4-(5-(cyclobutanecarboxamido)benzo[d]oxazol-2-yl)picolinic Acid (67B)

Methanol (5 mL) and sodium hydroxide (21 mg, 0.51 mmol) were successively added to compound 67A (150 mg, 0.43 mmol), and the reaction was stirred at room temperature for 2 hours. The solvent was subjected to rotary evaporation, and the residue was adjusted to pH=4-5 by dropwise adding 1 M hydrochloric acid solution. The solid was rinsed with a small amount of water and concentrated under reduced pressure to obtain compound 67B (137 mg, 91%).

LC-MS (ESI): m/z=338.1[M+H]⁺.

Step 3:

### N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclobutanecarboxamide (Compound 67C)

Compound 67B (137 mg, 0.44 mmol) was dissolved in N,N-dimethylformamide (2 mL). HATU (251.0 mg, 0.66 mmol) and DIPEA (171.6 mg, 1.32 mmol) were successively added, and then intermediate 6 (142.1 mg, 0.48 mmol) was added. The mixture was reacted at room temperature for one hour. The reaction solution was poured into water (50 mL) and extracted with ethyl acetate (20 mL×2). The organic phase was concentrated, and the residue was separated and purified by column chromatography (DCM/MeOH=20:1) to obtain compound 67C (120 mg, 45.9%).

Step 4:

### (R)—N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclobutanecarboxamide (Compound 67)

### (S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclobutanecarboxamide (Compound 68)

Compound 67C was subjected to SFC chiral preparative separation to obtain compound 67 (23 mg, 15%, a compound in a single configuration) and compound 68 (31 mg, 21%, a compound in a single configuration).

Preparation method: (instrument name: Waters UPC2 analytical SFC (SFC-L); chromatographic column: ChiralPak AD, 150×4.6 mm I.D., 3 μm; mobile phase: A for CO₂ and B for isopropanol (0.05% DEA); gradient: B 50%; flow rate: 2.5 mL/min; column pressure: 100 bar; column temperature: 35° C.; absorption wavelength: 220 nm; cycle time: about 10 min); retention time for compound 67: 5.838 min; retention time for compound 68: 8.122 min. The absolute structures of compound 67 and compound 68 are uncertain.

Compound 67: LC-MS (ESI): m/z=614.2 [M+H]⁺.

¹H NMR (400 MHz, DMSO-d6) δ 9.95 (s, 1H), 8.80 (dd, 1H), 8.55 (s, 1H), 8.54 (t, 1H), 8.26 (d, 1H), 8.21-8.09 (m, 1H), 7.77 (d, 1H), 7.61 (dd, 1H), 7.49 (d, 2H), 7.44-7.29 (m, 2H), 5.31 (s, 1H), 3.71 (s, 2H), 3.49 (s, 2H), 3.25 (d, 1H), 2.58 (s, 1H), 2.36 (s, 1H), 2.30-2.20 (m, 1H), 2.13 (d, 1H), 2.06-1.74 (m, 2H), 1.24 (s, 1H), 1.04 (d, 1H).

Compound 68: LC-MS (ESI): m/z=614.2 [M+H]⁺.

¹H NMR (400 MHz, DMSO-d6) δ 9.95 (s, 1H), 8.80 (dd, 1H), 8.55 (s, 1H), 8.54 (t, 1H), 8.26 (d, 1H), 8.21-8.09 (m, 1H), 7.77 (d, 1H), 7.61 (dd, 1H), 7.49 (d, 2H), 7.44-7.29 (m, 2H), 5.31 (s, 1H), 3.71 (s, 2H), 3.49 (s, 2H), 3.25 (d, 1H), 2.58 (s, 1H), 2.36 (s, 1H), 2.30-2.20 (m, 1H), 2.13 (d, 1H), 2.06-1.74 (m, 2H), 1.24 (s, 1H), 1.04 (d, 1H).

### Example 69

### N-(2-(2-(4-((2-methyl-2H-tetrazol-5-yl)(phenyl)methyl)piperazine-1-carbonyl)pyridin-4-yl)imidazo[1,2-a]pyridin-6-yl)acetamide

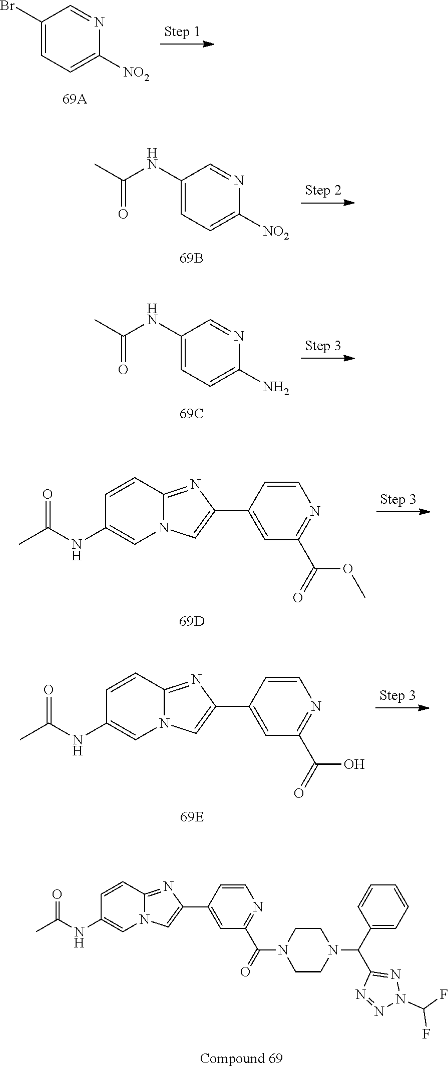

Step 1:

### N-(6-nitropyridin-3-yl)acetamide (69B)

Compound 69A (5.0 g, 24.6 mmol) was added to a 100 mL single-necked flask and dissolved in dioxane (50 mL). Acetamide (2.2 g, 26.9 mmol), Cs₂CO₃ (24.0 g, 73.9 mmol), Xantphos (4,5-bis(diphenylphosphino)-9,9-dimethylxanthene) (2.9 g, 4.9 mmol) and Pd₂(dba)₃ (2.3 g, 2.5 mmol) were added. The mixture was subjected to nitrogen replacement 3 times and reacted at 110° C. for 6 h. Water (100 mL) was added, and the resulting solution was extracted with EA (50 mL×3). The organic phase was combined, washed with saturated sodium chloride solution (100 mL×1), dried over anhydrous sodium sulfate and concentrated under reduced pressure, and the residue was separated and purified by column chromatography (PE/EA=3:1) to obtain compound 69B (3.7 g, yield: 70%).

¹H NMR (400 MHz, CDCl₃) δ 8.59 (dd, 1H), 8.52 (d, 1H), 8.28 (d, 1H), 2.29 (s, 3H).

Step 2:

### N-(6-aminopyridin-3-yl)acetamide (69C)

Compound 69B (2.0 g, 11.0 mmol) was added to a 100 mL single-necked flask and dissolved in methanol (20 mL). Pd—C (1.0 g) was added, and the mixture was subjected to hydrogen replacement 3 times, reacted under normal pressure at room temperature for 4 h, filtered through celite and concentrated under reduced pressure to obtain 69C (1.6 g, yield: 96%).

LC-MS (ESI): m/z=152.2 [M+H]⁺.

Step 3:

### Methyl 4-(6-acetamidoimidazo[1,2-a]pyridin-2-yl)picolinate (69D)

Compound 69C (0.6 g, 3.9 mmol) was added to a 100 mL single-necked flask and dissolved in MeOH (10 mL). Methyl 4-(2-bromoacetyl)picolinate (1.0 g, 3.9 mmol) was added, and the mixture was reacted at 60° C. for 6 h, concentrated, dissolved in EA (10 mL), washed with saturated sodium bicarbonate solution (10 mL), dried and concentrated, and the residue was separated and purified by column chromatography (PE/EA=1:1) to obtain 69D as a yellow oil (0.18 g, yield: 15%).

LC-MS (ESI): m/z=311.2 [M+H]⁺.

Step 4:

### 4-(6-acetamidoimidazo[1,2-a]pyridin-2-yl)picolinic Acid (69E)

Compound 69D (0.18 g, 0.58 mmol) was added to a 100 mL single-necked flask and dissolved in MeOH (3 mL), THE (3 mL) and water (3 mL). NaOH (0.12 g, 2.9 mmol) was added, and the mixture was stirred at room temperature for 3 h. The reaction solution was adjusted to pH=3-4 with HCl (1 M), filtered by suction and dried to obtain 69E as a white solid (0.15 g, yield: 87%).

LC-MS (ESI): m/z=297.2 [M+H]⁺.

Step 5:

### N-(2-(2-(4-((2-methyl-2H-tetrazol-5-yl)(phenyl)methyl)piperazine-1-carbonyl)pyridin-4-yl)imidazo[1,2-a]pyridin-6-yl)acetamide (Compound 69)

Compound 69E (0.12 g, 0.4 mmol) was added to a 100 mL single-necked flask and dissolved in DMF (3 mL). Intermediate 6 (0.12 g, 0.4 mmol), TEA (triethylamine) (0.12 g, 1.2 mmol) and HATU (0.18 g, 0.5 mmol) were added. The mixture was stirred at room temperature for 4 h, and water (10 mL) was added. The resulting solution was extracted with EA (10 mL×3). The organic phase was combined, washed with saturated sodium chloride solution (10 mL×1), dried over anhydrous sodium sulfate and concentrated under reduced pressure, and the residue was separated by HPLC to obtain compound 69 (80 mg, yield: 30%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.38 min.

LC-MS (ESI): m/z=573.3 [M+H]⁺.

¹H NMR (400 MHz, CDCl₃) δ 10.12 (s, 1H), 9.21 (dd, 1H), 8.75-8.67 (m, 1H), 8.60-8.53 (m, 2H), 7.98 (dd, 1H), 7.90 (dd, 1H), 7.60 (m, 1H), 7.53-7.45 (m, 2H), 7.43-7.28 (m, 3H), 7.21 (dd, 1H), 5.31 (s, 1H), 3.70 (t, 2H), 3.48 (t, 2H), 2.46 (s, 1H), 2.51-2.41 (m, 2H), 2.38-2.30 (m, 1H), 2.10 (s, 3H).

### Example 70

### N-(2-(2-(4-((2-(difluoromethyl)-2H-tetrazol-5-yl)(phenyl)methyl)piperazine-1-carbonyl)pyridin-4-yl)benzo[d]oxazol-6-yl)acetamide

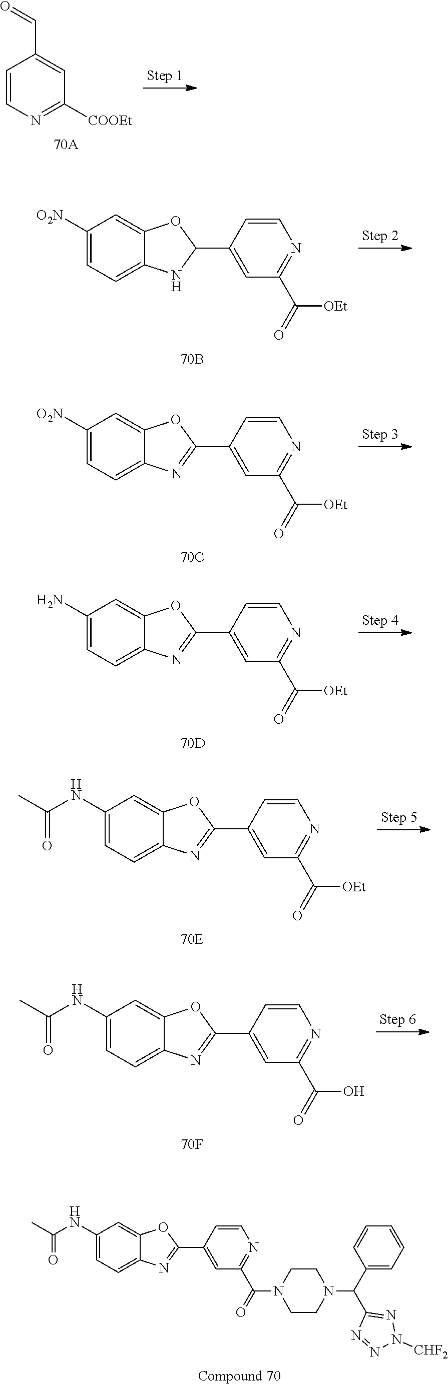

Step 1:

### Ethyl 4-(6-nitro-2,3-dihydrobenzo[d]oxazol-2-yl)picolinate (70B)

Compound 70A (1.0 g, 5.59 mmol) and 2-amino-5-nitrophenol (0.86 g, 5.59 mmol) were added to ethanol (20 mL), and the mixture was warmed to 70° C. and stirred for 15 h. The reaction was cooled to room temperature and then concentrated under reduced pressure to remove ethanol, and 70B (1.76 g, 99%) was obtained.

Step 2:

### Ethyl 4-(6-nitrobenzo[d]oxazol-2-yl)picolinate (70C)

Compound 70B (1.76 g, 5.59 mmol) was added to dichloromethane (20 mL), and then manganese dioxide (2.43 g, 27.95 mmol) was added. The mixture was stirred at 25° C. overnight. The resulting solution was filtered; the filtrate was concentrated; and then the residue was separated and purified by silica gel column chromatography (PE:EA=1:1) to obtain 70C (1.5 g, 86%).

LC-MS (ESI): m/z=314.1[M+H]⁺.

Step 3:

### Ethyl 4-(6-aminobenzo[d]oxazol-2-yl)picolinate (70D)

Compound 70C (0.40 g, 1.28 mmol) was added to methanol (50 mL); zinc powder (0.66 g, 10.2 mmol) and ammonium chloride (0.55 g, 10.2 mmol) were added; and the mixture was stirred at 25° C. for 3 hours. The resulting solution was filtered; the filtrate was concentrated; and the solid was washed with a small amount of water and dried to obtain 70D (0.30 g, 83%).

LC-MS (ESI): m/z=284.1 [M+H]⁺.

Step 4:

### Ethyl 4-(6-acetamidobenzo[d]oxazol-2-yl)picolinate (70E)

Compound 70D (300 mg, 1.06 mmol) was added to dichloromethane (10 mL), and then triethylamine (321 mg, 3.18 mmol) was added. The mixture was cooled to 0-5° C. in an ice bath, and acetyl chloride (250 mg, 3.18 mmol) was added. After the addition, the mixture was reacted for 2 hours; water (20 mL) was added; and the resulting solution was extracted twice with dichloromethane (20 mL×2). The organic phase was combined, dried and concentrated to obtain compound 70E (500 mg, 100%).

LC-MS (ESI): m/z=326.1 [M+H]⁺.

Step 5:

### 4-(6-acetamidobenzo[d]oxazol-2-yl)picolinic Acid (70F)

Anhydrous methanol (15 mL) and NaOH (0.25 g, 6.15 mmol, 2 mL) aqueous solution were successively added to compound 70E (0.5 g, 1.54 mmol). The mixture was stirred at room temperature overnight, concentrated under reduced pressure to remove most of methanol, adjusted to pH=2-3 by dropwise adding 1 N hydrochloric acid and filtered. The filter cake was washed with water (3 mL) and dried to obtain compound 70F (150 mg, 33%).

LC-MS (ESI): m/z=298.1 [M−H]⁻.

Step 6:

### N-(2-(2-(4-((2-(difluoromethyl)-2H-tetrazol-5-yl)(phenyl)methyl)piperazine-1-carbonyl)pyridin-4-yl)benzo[d]oxazol-6-yl)acetamide (Compound 70)

At room temperature, DMF (8 mL), intermediate 6 (148 mg, 0.51 mmol), HATU (291 mg, 0.77 mmol) and DIPEA (197 mg, 1.53 mmol) were successively added to compound 70F (150 mg, 0.51 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 70 (90 mg, 31%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.80 min.

LC-MS (ESI): m/z=574.3 [M+H]⁺.

¹H NMR (400 MHz, DMSO-d₆) δ 10.31 (s, 1H), 8.78-8.79 (m, 1H), 8.55 (t, 1H), 8.31-8.32 (m, 1H), 8.15-8.16 (m, 1H), 8.11-8.12 (m, 1H), 7.80 (d, 1H), 7.45-7.50 (m, 3H), 7.37-7.41 (m, 2H), 7.31-7.35 (m, 1H), 5.31 (s, 1H), 3.70-3.71 (m, 2H), 3.48-3.49 (m, 2H), 2.58-2.60 (m, 1H), 2.47-2.49 (m, 2H), 2.32-2.36 (m, 1H), 2.11 (s, 3H).

### Example 71

### (4-((2-(difluoromethyl)-2H-tetrazol-5-yl)(phenyl)methyl)piperazin-1-yl)(4-(5-(pyrrolidin-1-yl)benzo[d]oxazol-2-yl)pyridin-2-yl)methanone

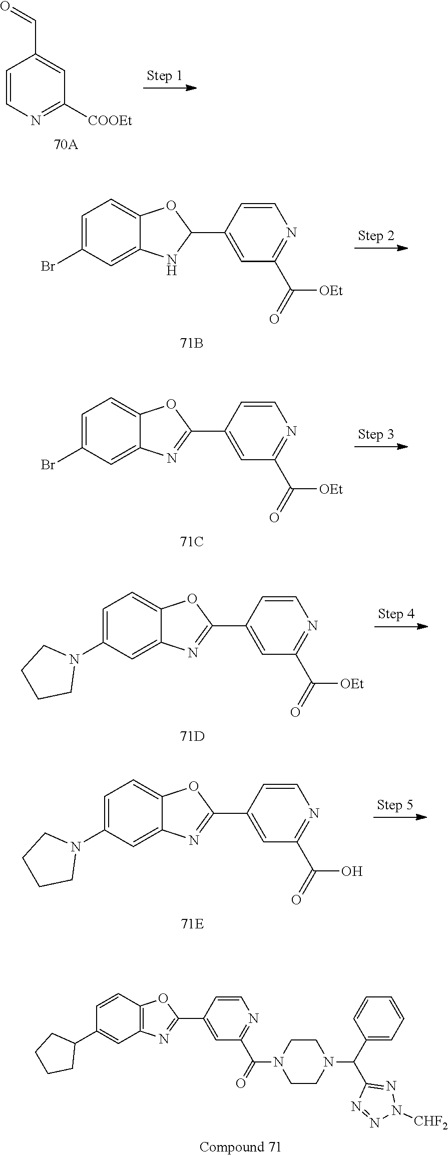 [SMILES: CCOC(=O)c1cc(C=O)ccn1 ; CCOC(=O)c1cc(C2Nc3cc(Br)ccc3O2)ccn1 ; CCOC(=O)c1cc(-c2nc3cc(Br)ccc3o2)ccn1 ; CCOC(=O)c1cc(-c2nc3cc(N4CCCC4)ccc3o2)ccn1 ; O=C(O)c1cc(-c2nc3cc(N4CCCC4)ccc3o2)ccn1 ; O=C(c1cc(-c2nc3cc(C4CCCC4)ccc3o2)ccn1)N1CCN(C(c2ccccc2)c2nnn(C(F)F)n2)CC1]

Step 1:

### Methyl 4-(5-bromo-2,3-dihydrobenzo[d]oxazol-2-yl)picolinate (71B)

Compound 70A (1.0 g, 5.59 mmol) and 2-amino-4-bromophenol (1.05 g, 5.59 mmol) were added to ethanol (20 mL), and the mixture was warmed to 75C and stirred for 15 h. The reaction was cooled to room temperature and then concentrated under reduced pressure to remove ethanol, and 71B (1.95 g, 990) was obtained.

Step 2:

### Methyl 4-(5-bromobenzo[d]oxazol-2-yl)picolinate (71C)

Compound 71B (1.95 g, 5.59 mmol) was added to dichloromethane (40 mL), and then manganese dioxide (2.43 g, 27.95 mmol) was added. The mixture was stirred at 25° C. overnight. The resulting solution was filtered; the filtrate was concentrated; and then the residue was separated and purified by silica gel column chromatography (PE:EA=1:1) to obtain 71C (1.5 g, 77%).

LC-MS (ESI): m/z=347.1 [M+H]⁺.

Step 3:

### Methyl 4-(5-(pyrrolidin-1l-yl)benzo[d]oxazol-2-yl)picolinate (71D)

Compound 71C (400 mg, 1.18 mmol), Pd₂(dba)₃ (63 mg, 0.06 mmol), potassium tert-butoxide (396 mg, 3.54 mmol), BINAP (1,1′-binaphthyl-2,2′-bis(diphenylphosphino)) (77 mg, 0.12 mmol) and tetrahydropyrrole (838 mg, 11.8 mmol) were added to dioxane (20 mL), and the mixture was stirred at 110° C. under nitrogen protection for 4 hours. The resulting solution was filtered; the filtrate was concentrated; and then the residue was separated and purified by silica gel column chromatography (PE:EA=1:2) to obtain 71D (200 mg, 50%).

LC-MS (ESI): m/z=338.1 [M+H]⁺.

Step 4:

### 4-(5-(pyrrolidin-1-yl)benzo[d]oxazol-2-yl)picolinic Acid (71E)

Anhydrous methanol (10 mL) and NaOH (95 mg, 2.37 mmol, 1 mL) aqueous solution were successively added to compound 71D (200 mg, 0.59 mmol). The mixture was stirred at room temperature overnight, concentrated under reduced pressure to remove most of methanol, adjusted to pH=2-3 by dropwise adding 1 N hydrochloric acid and filtered. The filter cake was washed with water (3 mL) and dried to obtain compound 71E (130 mg, 68%).

LC-MS (ESI): m/z=326.1 [M+H]⁺.

Step 5:

### (4-((2-(difluoromethyl)-2H-tetrazol-5-yl)(phenyl)methyl)piperazin-1-yl)(4-(5-(pyrrolidin-1-yl)benzo[d]oxazol-2-yl)pyridin-2-yl)methanone (Compound 71)

At room temperature, DMF (8 mL), intermediate 6 (118 mg, 0.40 mmol), HATU (228 mg, 0.60 mmol) and DIPEA (155 mg, 1.20 mmol) were successively added to compound 71E (130 mg, 0.40 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 71 (110 mg, 47%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.70 min.

LC-MS (ESI): m/z=586.3 [M+H]⁺.

¹H NMR (400 MHz, DMSO-d₆) δ 8.77-8.78 (m, 1H), 8.56 (t, 1H), 8.13-8.14 (m, 1H), 8.08-8.10 (m, 1H), 7.63 (d, 1H), 7.51-7.52 (m, 2H), 7.35-7.42 (m, 3H), 6.87-6.88 (m, 1H), 6.77-6.79 (m, 1H), 5.42 (s, 1H), 3.72-3.73 (m, 2H), 3.52-3.53 (m, 2H), 3.27-3.31 (m, 4H), 2.63-2.67 (m, 1H), 2.52-2.56 (m, 2H), 2.49-2.50 (m, 1H), 1.98-2.01 (m, 4H).

### Example 72

### (4-((2-(difluoromethyl)-2H-tetrazol-5-yl)(phenyl)methyl)piperazin-1-yl)(4-(5-(1-methyl-1H-imidazol-2-yl)benzo[d]oxazol-2-yl)pyridin-2-yl)methanone

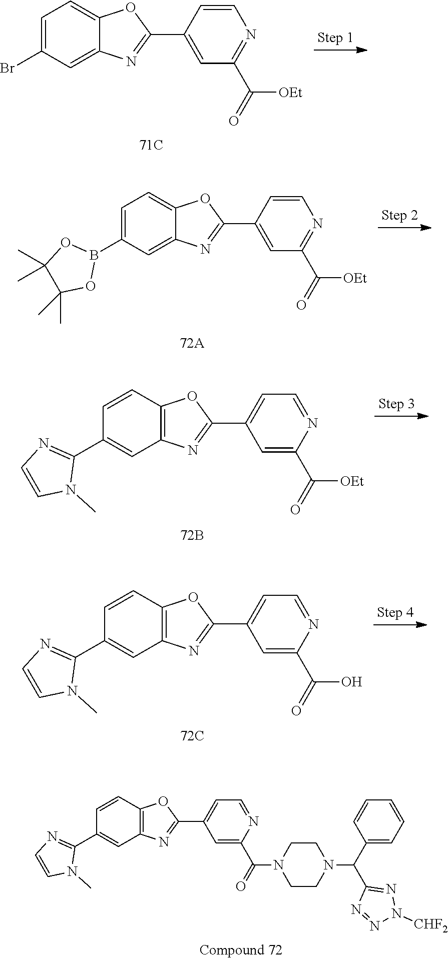 [SMILES: CCOC(=O)c1cc(-c2nc3cc(Br)ccc3o2)ccn1 ; CCOC(=O)c1cc(-c2nc3cc(B4OC(C)(C)C(C)(C)O4)ccc3o2)ccn1 ; CCOC(=O)c1cc(-c2nc3cc(-c4nccn4C)ccc3o2)ccn1 ; Cn1ccnc1-c1ccc2oc(-c3ccnc(C(=O)O)c3)nc2c1 ; Cn1ccnc1-c1ccc2oc(-c3ccnc(C(=O)N4CCN(C(c5ccccc5)c5nnn(C(F)F)n5)CC4)c3)nc2c1]

Step 1:

### Methyl 4-(5-(4,4,5,5-tetramethyl-1,3,2-dioxaborolan-2-yl)benzo[d]oxazol-2-yl)picolinate (72A)

Compound 71C (500 mg, 1.44 mmol) was dissolved in dioxane (20 mL), and then bis(pinacolato)diboron (732 mg, 2.88 mmol), potassium acetate (423 mg, 4.32 mmol) and Pd(dppf)Cl₂ (102 mg, 0.14 mmol) were added. The mixture was warmed to 100° C. and stirred for 5 h under nitrogen protection. The reaction was cooled to room temperature, and the filtrate was concentrated and then separated and purified by silica gel column chromatography (PE:EA=1:2) to obtain 72A (539 mg, 95%).

LC-MS (ESI): m/z=395.2[M+H]⁺.

Step 2:

### Methyl 4-(5-(1-methyl-1H-imidazol-2-yl)benzo[d]oxazol-2-yl)picolinate (72B)

Compound 72A (539 mg, 1.37 mmol) was added to a mixed solvent of dioxane (20 mL) and water (1 mL), and then 2-bromo-1-methyl-1H-imidazole (266 mg, 1.66 mmol), cesium carbonate (1082 mg, 3.32 mmol) and Pd(dppf)Cl₂ (102 mg, 0.14 mmol) were added. The mixture was warmed to 100° C. and stirred for 10 h under nitrogen protection. The reaction was cooled to room temperature, and the filtrate was concentrated and then separated and purified by silica gel column chromatography (PE:EA=1:3) to obtain 72B (150 mg, 31%).

LC-MS (ESI): m/z=349.1[M+H]⁺.

Step 3:

### 4-(5-(1-methyl-1H-imidazol-2-yl)benzo[d]oxazol-2-yl)picolinic Acid (72C)

Anhydrous methanol (8 mL) and NaOH (69 mg, 1.72 mmol, 0.5 mL) aqueous solution were successively added to compound 72B (150 mg, 0.43 mmol), and the mixture was stirred at room temperature overnight. The reaction solution was adjusted to pH=2-3 by dropwise adding 1 N hydrochloric acid, concentrated under reduced pressure to remove the solvent and dried to obtain a crude product of compound 72C, which was directly used in the next reaction (180 mg).

LC-MS (ESI): m/z=321.1 [M+H]⁺.

Step 4:

### (4-((2-(difluoromethyl)-2H-tetrazol-5-yl)(phenyl)methyl)piperazin-1-yl)(4-(5-(1-methyl-1H-imidazol-2-yl)benzo[d]oxazol-2-yl)pyridin-2-yl)methanone (Compound 72)

At room temperature, DMF (8 mL), intermediate 6 (148 mg, 0.51 mmol), HATU (291 mg, 0.77 mmol) and DIPEA (197 mg, 1.53 mmol) were successively added to compound 72C (163 mg, 0.51 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 72 (50 mg, 16%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.93 min.

LC-MS (ESI): m/z=597.3 [M+H]⁺.

¹H NMR (400 MHz, DMSO-d₆) δ 8.86-8.87 (m, 1H), 8.56 (t, 1H), 8.41-8.42 (m, 1H), 8.25-8.26 (m, 1H), 8.18-8.21 (m, 2H), 7.91-7.93 (m, 1H), 7.85-7.89 (m, 2H), 7.49-7.51 (m, 2H), 7.32-7.41 (m, 3H), 5.34 (s, 1H), 3.91 (s, 3H), 3.72-3.73 (m, 2H), 3.52-3.53 (m, 2H), 2.60-2.62 (m, 1H), 2.52-2.56 (m, 2H), 2.36-2.39 (m, 1H).

### Example 73

### (S)—N-(2-(2-(4-((R)-(2-(difluoromethyl)-2H-tetrazol-5-yl)(phenyl)methyl)piperazine-1-carbonyl)pyridin-4-yl)benzo[d]oxazol-5-yl)-2,2-difluorocyclopropane-1-carboxamide

### Example 74

### (S)—N-(2-(2-(4-((S)-(2-(difluoromethyl)-2H-tetrazol-5-yl)(phenyl)methyl)piperazine-1-carbonyl)pyridin-4-yl)benzo[d]oxazol-5-yl)-2,2-difluorocyclopropane-1-carboxamide

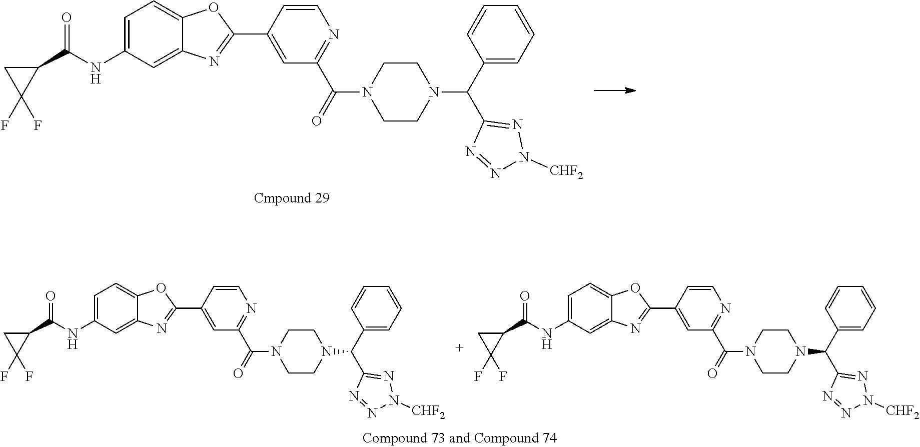 [SMILES: O=C(Nc1ccc2oc(-c3ccnc(C(=O)N4CCN(C(c5ccccc5)c5nnn(C(F)F)n5)CC4)c3)nc2c1)[C@@H]1CC1(F)F ; O=C(Nc1ccc2oc(-c3ccnc(C(=O)N4CCN([C@H](c5ccccc5)c5nnn(C(F)F)n5)CC4)c3)nc2c1)[C@@H]1CC1(F)F ; O=C(Nc1ccc2oc(-c3ccnc(C(=O)N4CCN([C@@H](c5ccccc5)c5nnn(C(F)F)n5)CC4)c3)nc2c1)[C@@H]1CC1(F)F]

Compound 29 was resolved by chiral HPLC to obtain compound 73 and compound 74 (chiral HPLC resolution method: instrument name: Waters UPC2 analytical SFC (SFC-H); chromatographic column: ChiralPak AD, 150×4.6 mm I.D., 3 μm; mobile phase: A for CO₂ and B for isopropanol (0.05% DEA); gradient: B 40%; flow rate: 2.5 mL/min; column pressure: 100 bar; column temperature: 35° C.; absorption wavelength: 220 nm; cycle time: about 20 min). retention time for compound 73: 10.89 min; retention time for compound 74: 14.49 min. Compound 73 and compound 74 are compounds with single configurations.

Compound 73: LC-MS (ESI): m/z=636.2 [M+H]⁺.

¹H NMR (400 MHz, CD₃OD) δ 8.79-8.80 (m, 1H), 8.33-8.34 (m, 1H), 8.20-8.22 (m, 2H), 8.19 (t, J=60 Hz, 1H), 7.69-7.71 (m, 1H), 7.57-7.61 (m, 3H), 7.35-7.43 (m, 3H), 5.41 (s, 1H), 3.91-3.92 (m, 2H), 3.61-3.62 (m, 2H), 2.66-2.85 (m, 5H), 2.08-2.17 (m, 1H), 1.81-1.90 (m, 1H).

Compound 74: LC-MS (ESI): m/z=636.2 [M+H]⁺.

¹H NMR (400 MHz, CD₃OD) δ 8.79-8.80 (m, 1H), 8.33-8.34 (m, 1H), 8.20-8.22 (m, 2H), 8.19 (t, J=60 Hz, 1H), 7.69-7.71 (m, 1H), 7.57-7.61 (m, 3H), 7.35-7.43 (m, 3H), 5.41 (s, 1H), 3.91-3.92 (m, 2H), 3.61-3.62 (m, 2H), 2.66-2.85 (m, 5H), 2.08-2.17 (m, 1H), 1.81-1.90 (m, 1H).

### Example 75

### (1S,2S)—N-(2-(2-(4-((R)-(2-(difluoromethyl)-2H-tetrazol-5-yl)(phenyl)methyl)piperazine-1-carbonyl)pyridin-4-yl)benzo[d]oxazol-5-yl)-2-fluorocyclopropane-1-carboxamide

### Example 76

### (1S,2S)—N-(2-(2-(4-((S)-(2-(difluoromethyl)-2H-tetrazol-5-yl)(phenyl)methyl)piperazine-1-carbonyl)pyridin-4-yl)benzo[d]oxazol-5-yl)-2-fluorocyclopropane-1-carboxamide

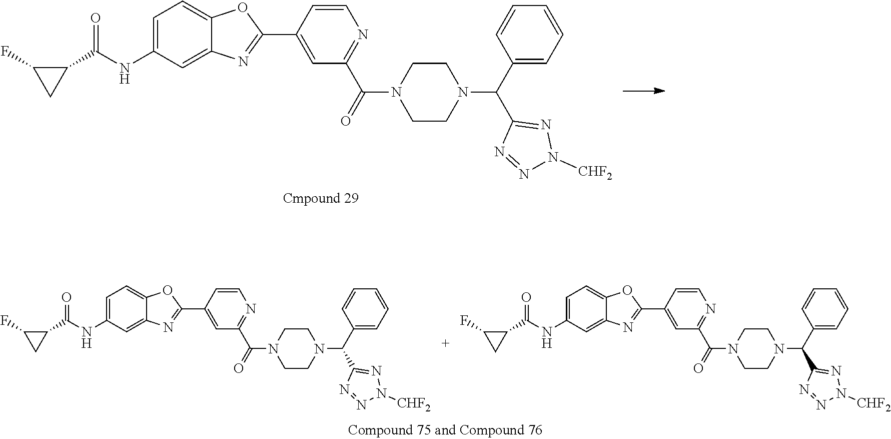

Compound 31 was resolved by chiral HPLC to obtain compound 75 and compound 76, with purification conditions as follows: (instrument name: Waters UPC2 analytical SFC (SFC-L); chromatographic column: ChiralPak AD, 150×4.6 mm I.D., 3 μm; mobile phase: A for CO₂ and B for isopropanol (0.05% DEA; gradient: B 50%; flow rate: 2.5 mL/min; column pressure: 100 bar; column temperature: 35° C.; absorption wavelength: 220 nm; cycle time: about 10 min); retention time for compound 75: 4.61 min; retention time for compound 76: 5.96 min. Compound 75 and compound 76 are compounds with single configurations.

Compound 75: LC-MS (ESI): m/z=618.3 [M+H]⁺.

¹H NMR (400 MHz, DMSO-d₆) δ 10.48 (s, 1H), 8.79-8.81 (m, 1H), 8.56 (t, J=56 Hz, 1H), 8.18-8.24 (m, 2H), 8.13-8.15 (m, 1H), 7.78-7.81 (m, 1H), 7.61-7.63 (m, 1H), 7.50-7.52 (m, 2H), 7.32-7.42 (m, 3H), 5.38 (s, 1H), 4.85-5.06 (m, 1H), 3.71-3.73 (m, 2H), 3.49-3.51 (m, 2H), 2.59-2.63 (m, 1H), 2.49-2.52 (m, 2H), 2.41-2.43 (m, 1H), 2.01-2.08 (m, 1H), 1.62-1.72 (m, 1H), 1.13-1.21 (m, 1H).

Compound 76: LC-MS (ESI): m/z=618.3 [M+H]⁺.

¹H NMR (400 MHz, DMSO-d₆) δ 10.48 (s, 1H), 8.79-8.81 (m, 1H), 8.56 (t, J=56 Hz, 1H), 8.18-8.24 (m, 2H), 8.13-8.15 (m, 1H), 7.78-7.81 (m, 1H), 7.61-7.63 (m, 1H), 7.50-7.52 (m, 2H), 7.32-7.42 (m, 3H), 5.38 (s, 1H), 4.85-5.06 (m, 1H), 3.71-3.73 (m, 2H), 3.49-3.51 (m, 2H), 2.59-2.63 (m, 1H), 2.49-2.52 (m, 2H), 2.41-2.43 (m, 1H), 2.01-2.08 (m, 1H), 1.62-1.72 (m, 1H), 1.13-1.21 (m, 1H).

### Example 77

### N-(2-(2-(4-(cyclopentyl(2-methyl-2H-tetrazol-5-yl)methyl)piperazine-1-carbonyl)pyridin-4-yl)benzo[d]oxazol-5-yl)acetamide

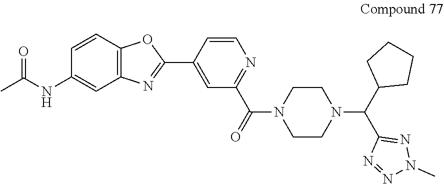 [SMILES: CC(=O)Nc1ccc2oc(-c3ccnc(C(=O)N4CCN(C(c5nnn(C)n5)C5CCCC5)CC4)c3)nc2c1]

Cyclopentanecarbaldehyde was used as a raw material to obtain compound 77 according to the synthetic step of compound 5.

¹H NMR (400 MHz, CDCl3) δ 8.71 (d, 1H), 8.22 (s, 1H), 8.04 (d, 1H), 7.95 (s, 1H), 7.79 (s, 1H), 7.47 (s, 2H), 4.37 (s, 3H), 4.04-3.41 (m, 5H), 2.84-2.38 (m, 4H), 2.20 (s, 3H), 2.03-1.85 (m, 1H), 1.60-1.42 (m, 6H), 1.31 (s, 1H), 0.97 (s, 1H).

LC-MS (ESI): m/z=530.3 [M+H]⁺.

### Example 78

### (1S,2S)—N-(2-(2-(4-((2-cyclopropyl-2H-tetrazol-5-yl)(phenyl)methyl)piperazine-1-carbonyl)pyridin-4-yl)benzo[d]oxazol-5-yl)-2-fluorocyclopropanecarboxamide

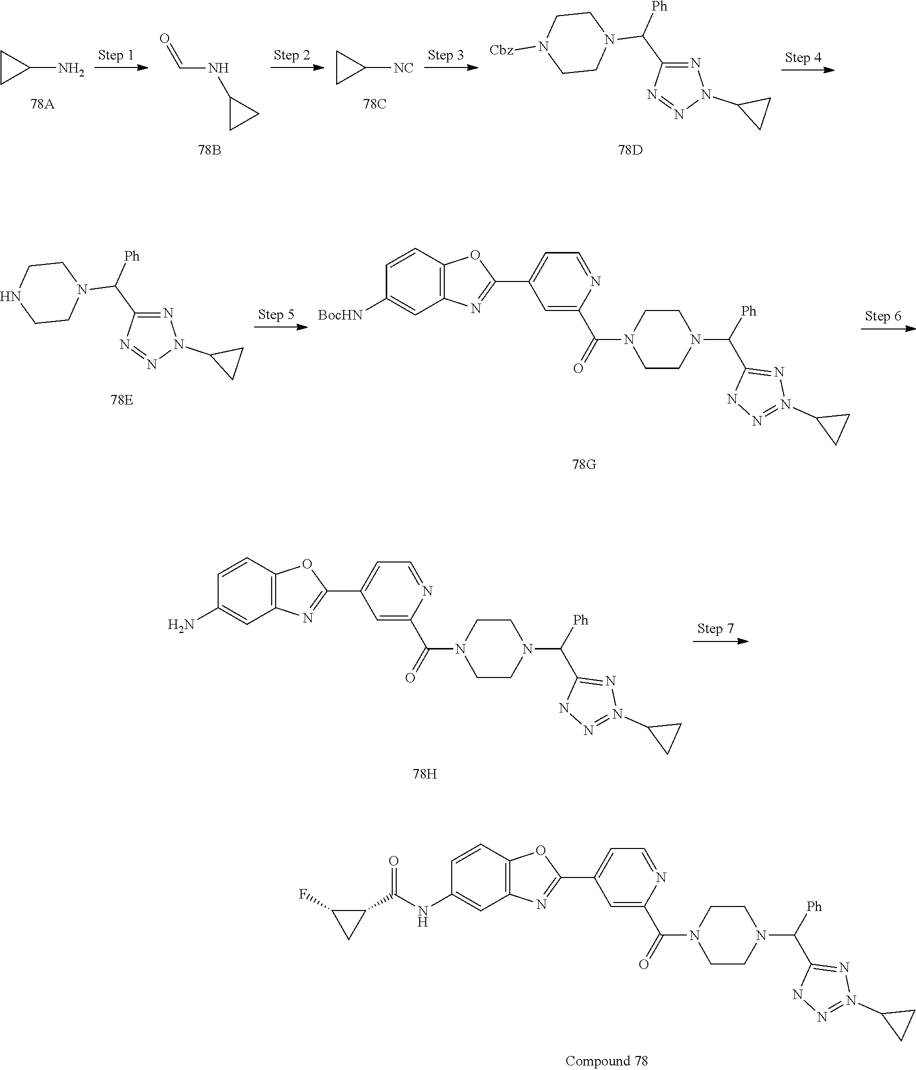

Step 1:

### N-cyclopropylformamide (78B)

Compound 78A (5 g, 87.7 mmol) was added to ethyl formate (30 mL), and the mixture was reacted at 55° C. for 16 h and directly concentrated under reduced pressure to obtain 78B (6 g, 80.5%).

Step 2:

### Isocyanocyclopropane (78C)

78B (5.2 g, 61.2 mmol) was added to dichloromethane (40 ml), and then Burgess reagent (17 g, 73.4 mmol) was added slowly. The mixture was reacted at room temperature for 3 h. After completion of the reaction, the reaction solution was directly used in the next reaction.

Step 3:

### benzyl4-((2-cyclopropyl-2H-tetrazol-5-yl)(phenyl)methyl)piperazine-1-carboxylate (78D)

Benzyl piperazine-1-carboxylate (8.8 g, 40 mmol) and benzaldehyde (4.24 g, 40 mmol) were added to methanol (50 ml), and the mixture was stirred for 10 min. Then azidotrimethylsilane (6.9 g, 60 mmol) and 78C (dichloromethane solution) were added, and the mixture was reacted at room temperature for 3 h. 150 ml of water was added to the reaction system, and the resulting solution was extracted with dichloromethane (50 mL×3). The organic phase was combined, dried over anhydrous sodium sulfate and filtered. The filtrate was concentrated under reduced pressure, and then the residue was separated and purified by silica gel column chromatography (eluent: PE/EA=1/2) to obtain 78D (2.4 g, 14%).

LC-MS (ESI): m/z=419.2 [M+H]⁺.

Step 4:

### 1-((2-cyclopropyl-2H-tetrazol-5-yl)(phenyl)methyl)piperazine (78E)

10% palladium on carbon (300 mg) and methanol (30 ml) were added to 78D (2.4 g, 5.7 mmol). After hydrogen replacement, the mixture was reacted at room temperature for 4 h. After completion of the reaction, the reaction mixture was filtered through celite. The filtrate was concentrated under reduced pressure to obtain 78E (1.6 g, 99%).

LC-MS (ESI): m/z=285.2 [M+H]⁺.

Step 5:

### tert-butyl(2-(2-(4-((2-cyclopropyl-2H-tetrazol-5-yl)(phenyl)methyl)piperazine-1-carbonyl)pyridin-4-yl)benzo[d]oxazol-5-yl)carbamate (78G)

78E (351 mg, 1.24 mmol), DMF (5 ml), HATU (640 mg, 1.68 mmol) and DIPEA (361 mg, 2.8 mmol) were successively added to 24A (400 mg, 1.12 mmol). The mixture was reacted at room temperature for 2 h, and water (30 mL) was added to the reaction solution. The resulting solution was extracted with ethyl acetate (30 mL×2). The combined organic phase was dried over anhydrous sodium sulfate and concentrated under reduced pressure, and then the residue was separated and purified by silica gel column chromatography (eluent: DCM/EA=1/2) to obtain 78G (520 mg, 75%).

LC-MS (ESI): m/z=622.3 [M+H]⁺.

Step 6:

### (4-(5-aminobenzo[d]oxazol-2-yl)pyridin-2-yl)(4-((2-cyclopropyl-2H-tetrazol-5-yl)(phenyl)methyl)piperazin-1-yl)methanone Hydrochloride (78H)

Hydrochloric acid in dioxane (10 ml) was added to 78G (520 mg, 0.84 mmol), and the mixture was reacted at room temperature for 3 h. After completion of the reaction, the reaction solution was concentrated under reduced pressure to obtain 78H (456 mg, 97.6%).

Step 7:

### (1S,2S)—N-(2-(2-(4-((2-cyclopropyl-2H-tetrazol-5-yl)(phenyl)methyl)piperazine-1-carbonyl)pyridin-4-yl)benzo[d]oxazol-5-yl)-2-fluorocyclopropanecarboxamide (Compound 78)

78H (250 mg, 0.38 mmol) was dissolved in N,N-dimethylformamide (2 mL). HATU (90 mg, 0.234 mmol), DIPEA (75 mg, 0.585 mmol) and (1S,2S)-2-fluorocyclopropanecarboxylic acid (40 mg 0.38 mmol) were added. After the addition, the mixture was stirred for 1 hour at room temperature, and then the reaction was quenched by adding water (20 mL). The resulting solution was extracted twice with ethyl acetate (20 mL×2). The organic phase was combined, dried over anhydrous sodium sulfate and concentrated under reduced pressure, and then the residue was separated by column chromatography (eluent: DCM/EA=1/3) to obtain compound 78 (100 mg, 43%).

¹H NMR (400 MHz, CD₃OD) δ 8.79-8.77 (m, 1H), 8.30-8.29 (m, 1H), 8.21-8.15 (m, 2H), 7.68-7.66 (m, 1H), 7.60-7.57 (m, 1H), 7.50-7.48 (m, 2H), 7.42-7.33 (m, 3H), 5.30 (s, 1H), 4.79-4.73 (m, 1H), 3.88-3.86 (m, 2H), 3.74-3.68 (m, 1H), 3.60-3.59 (m, 2H), 2.77-2.74 (m, 1H), 2.70-2.59 (m, 2H), 2.56-2.48 (m, 1H), 2.05-1.98 (m, 1H), 1.84-1.73 (m, 1H), 1.34-1.24 (m, 2H), 1.21-1.12 (m, 2H), 1.08-1.01 (m, 1H).

LC-MS (ESI): m/z=608.2[M+H]⁺.

### Example 79

### (R/S)—N-(2-(2-(4-((2-(difluoromethyl)-2H-tetrazol-5-yl)(phenyl)methyl)piperazine-1-carbonyl)-3-fluoropyridin-4-yl)benzo[d]oxazol-5-yl)acetamide

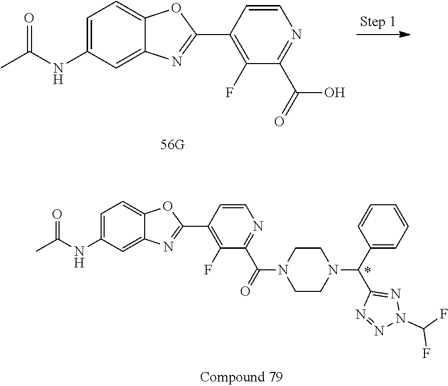 [SMILES: CC(=O)Nc1ccc2oc(-c3ccnc(C(=O)O)c3F)nc2c1 ; CC(=O)Nc1ccc2oc(-c3ccnc(C(=O)N4CCN(C(c5ccccc5)c5nnn(C(F)F)n5)CC4)c3F)nc2c1]

Step 1:

### (R/S)—N-(2-(2-(4-((2-(difluoromethyl)-2H-tetrazol-5-yl)(phenyl)methyl)piperazine-1-carbonyl)-3-fluoropyridin-4-yl)benzo[d]oxazol-5-yl)acetamide (Compound 79)

Intermediate 7 (130 mg, 0.44 mmol) and 56G (153 mg, 0.485 mmol) were dissolved in N,N-dimethylformamide (5 mL). HATU (250 mg, 0.66 mmol) and DIPEA (170 mg, 1.32 mmol) were added. After the addition, the mixture was stirred for 1 hour at room temperature, and then the reaction was quenched by adding water (30 mL). The resulting solution was extracted twice with ethyl acetate (30 mL×2). The organic phase was combined, dried over anhydrous sodium sulfate and concentrated under reduced pressure, and then the residue was separated by column chromatography (eluent: MeOH/DCM=1/20) to obtain compound 79 (110 mg, 42%, a compound in a single configuration).

¹H NMR (400 MHz, MeOD) δ 8.60 (d, 1H), 8.30-8.02 (m, 3H), 7.69-7.67 (m, 1H), 7.60-7.58 (m, 1H), 7.55-7.49 (m, 2H), 7.39-7.27 (m, 3H), 5.20 (s, 1H), 3.87 (t, 2H), 3.42 (t, 2H), 2.76-2.67 (m, 1H), 2.63-2.52 (m, 2H), 2.47-2.39 (m, 1H), 2.17 (s, 3H).

LC-MS (ESI): m/z=592.2[M+H]⁺.

### Example 80

### 1-methyl-N-(2-(2-(4-((2-methyl-2H-tetrazol-5-yl)(phenyl)methyl)piperazine-1-carbonyl)pyridin-4-yl)benzo[d]oxazol-5-yl)-1H-imidazole-5-carboxamide

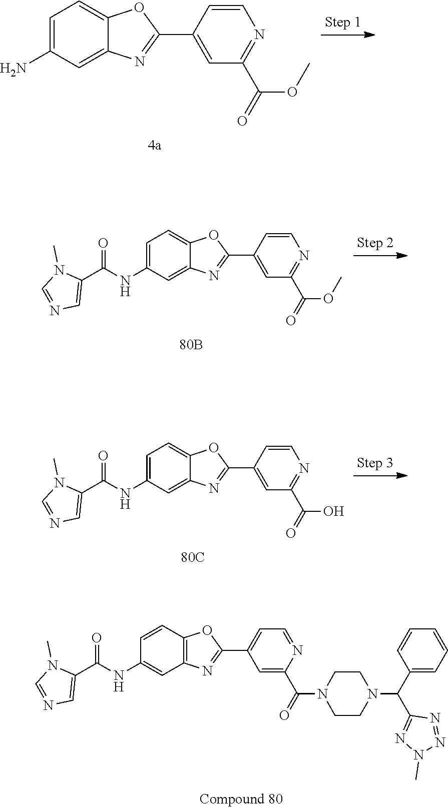 [SMILES: COC(=O)c1cc(-c2nc3cc(N)ccc3o2)ccn1 ; COC(=O)c1cc(-c2nc3cc(NC(=O)c4cncn4C)ccc3o2)ccn1 ; Cn1cncc1C(=O)Nc1ccc2oc(-c3ccnc(C(=O)O)c3)nc2c1 ; Cn1nnc(C(c2ccccc2)N2CCN(C(=O)c3cc(-c4nc5cc(NC(=O)c6cncn6C)ccc5o4)ccn3)CC2)n1]

### Step 1: Methyl 4-(5-(1-methyl-1H-imidazole-5-carboxamido)benzo[d]oxazol-2-yl)picolinate (80B)

Compound 4a (0.7 g, 2.6 mmol), 1-methyl-1H-imidazole-5-carboxylic acid (0.4 g, 3.1 mmol) and DIPEA (1.0 g, 7.8 mmol) were dissolved in DMF (15 mL). HATU (1.5 g, 3.9 mmol) was added. After the addition was completed, the mixture was stirred at room temperature overnight; water (20 mL) was added to the reaction solution; and the resulting solution was extracted 5 times with ethyl acetate. The organic phase was combined, dried over anhydrous sodium sulfate and filtered. The filtrate was evaporated to dryness under reduced pressure. The residue was purified by silica gel column chromatography (eluent: PE/EA=1/1) to obtain compound 80B (0.7 g, 68.7%).

Step 2:

### 4-(5-(1-methyl-1H-imidazole-5-carboxamido)benzo[d]oxazol-2-yl)picolinic Acid (80C)

Compound 80B (0.7 g, 1.9 mmol) was dissolved in methanol (8 ml) and water (4 ml), and NaOH (0.2 g, 5.7 mmol) was added. The mixture was stirred at room temperature for 2 hours, and then methanol was distilled off under reduced pressure. The aqueous phase was adjusted to pH value=5 with 2 N dilute hydrochloric acid and filtered. The filter cake was dried to obtain 80C (0.5 g, 89.6%).

LC-MS (ESI): m/z=364.2[M+H]⁺.

Step 3:

### 1-methyl-N-(2-(2-(4-((2-methyl-2H-tetrazol-5-yl)(phenyl)methyl)piperazine-1-carbonyl)pyridin-4-yl)benzo[d]oxazol-5-yl)-1H-imidazole-5-carboxamide (Compound 80)

80C (65 mg, 0.25 mmol), intermediate 2 (110 mg, 0.30 mmol) and DIPEA (97 mg, 0.75 mmol) were dissolved in DMF (5 ml). HATU (144 mg, 0.38 mmol) was added. After the addition was completed, the mixture was stirred at room temperature overnight, and the reaction solution was filtered. The filtrate was purified by preparative HPLC to obtain compound 80 (102 mg, 67.5%). Preparation method: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.03 min.

¹H NMR (400 MHz, DMSO-d⁶) δ 10.25 (s, 1H), 8.82-8.80 (m, 1H), 8.19 (s, 1H), 8.15 (m, 1H), 7.85-7.82 (m, 3H), 7.78-7.75 (m, 1H), 7.49 (d, 1H), 7.38-7.34 (m, 2H), 7.31-7.27 (m, 1H), 5.10 (s, 1H), 4.35 (s, 3H), 3.89 (s, 3H), 3.70 (brs, 2H), 3.48 (brs, 2H), 2.61-2.58 (m, 1H), 2.49-2.44 (m, 2H), 2.38-2.33 (m, 1H).

LC-MS (ESI): m/z=604.3[M+H]⁺.

### Example 81

### N-(2-(2-(4-((2-(difluoromethyl)-2H-tetrazol-5-yl)(phenyl)methyl)piperazine-1-carbonyl)pyridin-4-yl)benzo[d]oxazol-5-yl)-1-methyl-1H-imidazole-5-carboxamide

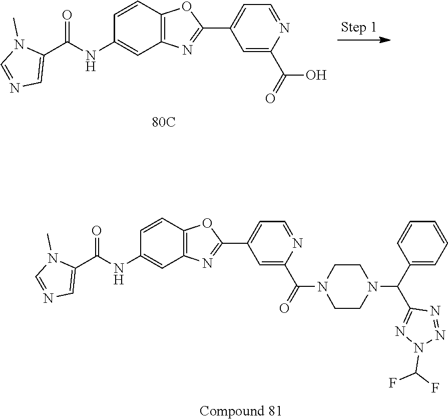 [SMILES: Cn1cncc1C(=O)Nc1ccc2oc(-c3ccnc(C(=O)O)c3)nc2c1 ; Cn1cncc1C(=O)Nc1ccc2oc(-c3ccnc(C(=O)N4CCN(C(c5ccccc5)c5nnn(C(F)F)n5)CC4)c3)nc2c1]

Compound 80C (200 mg, 0.55 mmol), intermediate 6 (162 mg, 0.55 mmol) and DIPEA (213 mg, 1.65 mmol) were dissolved in DMF (7 ml). HATU (314 mg, 0.83 mmol) was added. After the addition was completed, the mixture was stirred at room temperature overnight, and the reaction solution was filtered. The filtrate was purified by preparative HPLC to obtain compound 81 (213 mg, 60.7%). Preparation method: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.7 min.

¹H NMR (400 MHz, DMSO-ad) δ 10.25 (s, 1H), 8.82-8.80 (m, 1H), 8.56 (t, 1H), 8.29 (d, 1H), 8.19 (s, 1H), 8.15-8.14 (m, 1H), 7.85-7.82 (m, 3H), 7.78-7.75 (m, 1H), 7.51-7.49 (m, 2H), 7.41-7.37 (m, 2H), 7.35-7.31 (m, 1H), 5.32 (s, 1H), 3.89 (s, 3H), 3.89 (s, 3H), 3.72 (brs, 2H), 3.50 (brs, 2H), 2.58-2.55 (m, 1H), 2.48-2.43 (m, 2H), 2.34-2.31 (m, 1H).

LC-MS (ESI): m/z=640.3 [M+H]⁺.

### Example 82

### (R/S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-5-yl)-2,2-difluorocyclopropane-1-carboxamide

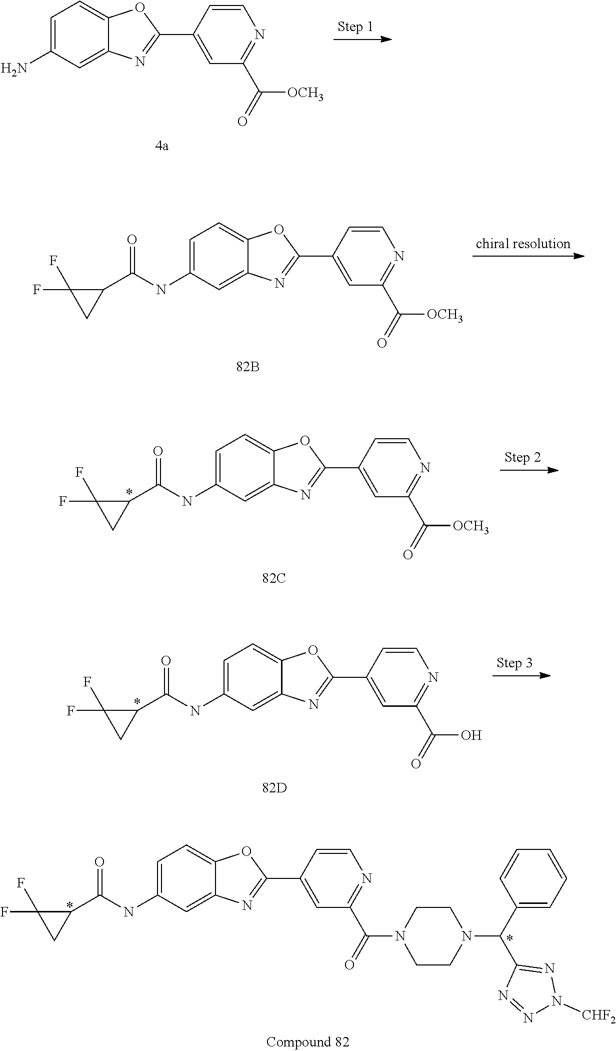 [SMILES: COC(=O)c1cc(-c2nc3cc(N)ccc3o2)ccn1 ; COC(=O)c1cc(-c2nc3cc(NC(=O)C4CC4(F)F)ccc3o2)ccn1 ; COC(=O)c1cc(-c2nc3cc(NC(=O)C4CC4(F)F)ccc3o2)ccn1 ; O=C(O)c1cc(-c2nc3cc(NC(=O)C4CC4(F)F)ccc3o2)ccn1 ; O=C(Nc1ccc2oc(-c3ccnc(C(=O)N4CCN(C(c5ccccc5)c5nnn(C(F)F)n5)CC4)c3)nc2c1)C1CC1(F)F]

Step 1:

### Methyl (R/S)-4-(5-(2,2-difluorocyclopropane-1-carboxamido)benzo[d]oxazol-2-yl)picolinate (82C)

At room temperature, DMF (15 mL), 2,2-difluorocyclopropane-1-carboxylic acid (454 mg, 3.72 mmol), HATU (2.12 g, 5.58 mmol) and DIPEA (1.44 g, 11.16 mmol) were successively added to compound 4a (1.0 g, 3.72 mmol). The reaction was stirred for 1 hour. The reaction solution was poured into water (50 mL) and extracted with ethyl acetate (20 mL×3). The organic phase was concentrated, and the residue was separated and purified by silica gel column chromatography to obtain compound 82B (1.2 g). 82B was purified and separated by chiral HPLC to obtain 82C (400 mg, a compound in a single configuration). (instrument name: Thar 200 preparative SFC (SFC-10); chromatographic column: ChiralPak AD, 300×50 mm I.D., 10 μm; mobile phase: A for CO₂ and B for Methanol; gradient: B 45%; flow rate: 200 mL/min; column pressure: 100 bar; column temperature: 38° C.; absorption wavelength: 220 nm; cycle time: about 7 min; retention time: 5.88 min).

LC-MS (ESI): m/z=374.1 [M+H]⁺.

Step 2:

### (R)-4-(5-(2,2-difluorocyclopropane-1-carboxamido)benzo[d]oxazol-2-yl)picolinic Acid (82D)

Anhydrous methanol (10 mL) and NaOH (215 mg, 5.36 mmol, 2 mL) aqueous solution were successively added to compound 82C (0.4 g, 1.07 mmol). The mixture was stirred at room temperature for 10 h, concentrated under reduced pressure to remove most of methanol, adjusted to pH=2-3 by dropwise adding 1 N hydrochloric acid and filtered. The filter cake was washed with water (3 mL) and dried to obtain intermediate 82D (300 mg, 78%).

LC-MS (ESI): m/z=360.1 [M+H]⁺.

Step 3:

### (R/S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-5-yl)-2,2-difluorocyclopropane-1-carboxamide (Compound 82)

At room temperature, DMF (5 mL), intermediate 7 (82 mg, 0.28 mmol), HATU (138 mg, 0.36 mmol) and DIPEA (100 mg, 0.84 mmol) were successively added to compound 82D (100 mg, 0.28 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 82 (40 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.99 min.

LC-MS (ESI): m/z=636.3 [M+H]⁺.

¹H NMR (400 MHz, DMSO-d₆) δ 10.65 (s, 1H), 8.80-8.81 (m, 1H), 8.55 (t, 1H), 8.21-8.22 (m, 1H), 8.17-8.18 (m, 1H), 8.13-8.15 (m, 1H), 7.82 (d, 1H), 7.59-7.62 (m, 1H), 7.48-7.50 (m, 2H), 7.37-7.41 (m, 2H), 7.31-7.34 (m, 1H), 5.31 (s, 1H), 3.70-3.71 (m, 2H), 3.48-3.49 (m, 2H), 2.80-2.88 (m, 1H), 2.57-2.60 (m, 1H), 2.43-2.47 (m, 2H), 2.32-2.37 (m, 1H), 1.99-2.09 (m, 2H).

### Example 83

### (1R)-2,2-difluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide

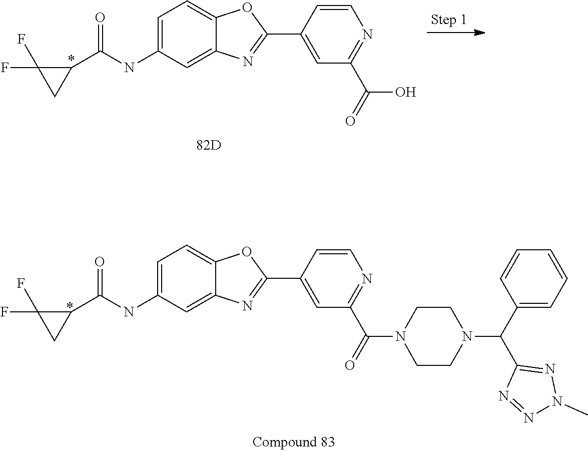 [SMILES: O=C(O)c1cc(-c2nc3cc(NC(=O)C4CC4(F)F)ccc3o2)ccn1 ; Cn1nnc(C(c2ccccc2)N2CCN(C(=O)c3cc(-c4nc5cc(NC(=O)C6CC6(F)F)ccc5o4)ccn3)CC2)n1]

At room temperature, DMF (5 mL), intermediate 2 (72 mg, 0.28 mmol), HATU (138 mg, 0.36 mmol) and DIPEA (100 mg, 0.84 mmol) were successively added to compound 82D (100 mg, 0.28 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 83 (35 mg, 21%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.85 min.

LC-MS (ESI): m/z=600.3 [M+H]⁺.

¹H NMR (400 MHz, DMSO-d₆) δ 10.65 (s, 1H), 8.80-8.81 (m, 1H), 8.21-8.22 (m, 1H), 8.15-8.17 (m, 1H), 8.13-8.15 (m, 1H), 7.82 (d, 1H), 7.59-7.62 (m, 1H), 7.48-7.50 (m, 2H), 7.34-7.37 (m, 2H), 7.27-7.31 (m, 1H), 5.10 (s, 1H), 4.35 (s, 3H), 3.68-3.69 (m, 2H), 3.47-3.48 (m, 2H), 2.80-2.88 (m, 1H), 2.51-2.58 (m, 1H), 2.43-2.47 (m, 2H), 2.30-2.33 (m, 1H), 1.98-2.09 (m, 2H).

### Example 84

### 2-(2-(4-((2-(difluoromethyl)-2H-tetrazol-5-yl)(phenyl)methyl)piperazine-1-carbonyl)pyridin-4-yl)-N-methylbenzo[d]oxazole-5-carboxamide

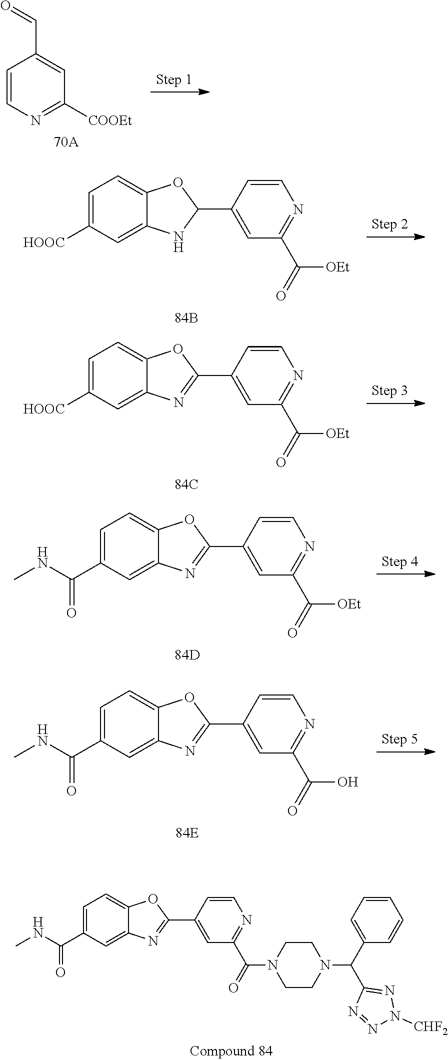 [SMILES: CCOC(=O)c1cc(C=O)ccn1 ; CCOC(=O)c1cc(C2Nc3cc(C(=O)O)ccc3O2)ccn1 ; CCOC(=O)c1cc(-c2nc3cc(C(=O)O)ccc3o2)ccn1 ; CCOC(=O)c1cc(-c2nc3cc(C(=O)NC)ccc3o2)ccn1 ; CNC(=O)c1ccc2oc(-c3ccnc(C(=O)O)c3)nc2c1 ; CNC(=O)c1ccc2oc(-c3ccnc(C(=O)N4CCN(C(c5ccccc5)c5nnn(C(F)F)n5)CC4)c3)nc2c1]

Step 1:

### 2-(2-(ethoxycarbonyl)pyridin-4-yl)-2,3-dihydrobenzo[d]oxazole-5-carboxylic Acid (84B)

Compound 70A (1.0 g, 5.59 mmol) and 3-amino-4-hydroxyl benzoic acid (0.85 g, 5.59 mmol) were added to ethanol (20 mL), and the mixture was warmed to 70° C. and stirred for 15 h. The reaction was cooled to room temperature and then concentrated under reduced pressure to remove ethanol, and 84B (0.85 g, 48%) was obtained.

Step 2:

### 2-(2-(ethoxycarbonyl)pyridin-4-yl)benzo[d]oxazole-5-carboxylic Acid (84C)

Compound 84B (0.85 g, 2.71 mmol) was added to dichloromethane (1 L), and then manganese dioxide (1.18 g, 13.55 mmol) was added. The mixture was stirred at 25° C. overnight. The resulting solution was filtered; the filtrate was concentrated; and then the residue was separated and purified by silica gel column chromatography (DCM:MeOH=10:1) to obtain 84C (0.65 g, 77%).

LC-MS (ESI): m/z=313.1[M+H]⁺.

Step 3:

### Ethyl 4-(5-(methylcarbamoyl)benzo[d]oxazol-2-yl)picolinate (84D)

At room temperature, DMF (20 mL), methylamine hydrochloride (708 mg, 10.42 mmol), HATU (1185 mg, 3.12 mmol) and DIPEA (2683 mg, 20.8 mmol) were successively added to compound 84C (650 mg, 2.08 mmol). The reaction was stirred for 2 hours. The reaction solution was poured into water (70 mL) and extracted with ethyl acetate (50 mL×3). The organic phase was concentrated, and the residue was separated and purified by silica gel column chromatography (PE:EA=1:5) to obtain 84D (200 mg, 30%).

LC-MS (ESI): m/z=326.1 [M+H]⁺.

Step 4:

### 4-(5-(methylcarbamoyl)benzo[d]oxazol-2-yl)picolinic Acid (84E)

Anhydrous methanol (10 mL) and NaOH (123 mg, 3.08 mmol, 2 mL) aqueous solution were successively added to compound 84D (200 mg, 0.62 mmol). The mixture was stirred at room temperature overnight, concentrated under reduced pressure to remove most of methanol, adjusted to pH=2-3 by dropwise adding 1 N hydrochloric acid and filtered. The filter cake was washed with water (3 mL) and dried to obtain compound 84E (100 mg, 54%).

LC-MS (ESI): m/z=298.1 [M+H]⁺.

Step 5:

### 2-(2-(4-((2-(difluoromethyl)-2H-tetrazol-5-yl)(phenyl)methyl)piperazine-1-carbonyl)pyridin-4-yl)-N-methylbenzo[d]oxazole-5-carboxamide (Compound 84)

At room temperature, DMF (8 mL), intermediate 6 (99 mg, 0.34 mmol), HATU (194 mg, 0.51 mmol) and DIPEA (132 mg, 1.02 mmol) were successively added to compound 84E (100 mg, 0.34 mmol). The reaction was stirred for 1 hour. The reaction solution was poured into water (50 mL) and extracted with ethyl acetate (20 mL×3). The organic phase was concentrated, and the residue was separated and purified by HPLC to obtain compound 84 (40 mg, 21%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.88 min

LC-MS (ESI): m/z=574.3 [M+H]⁺.

¹H NMR (400 MHz, DMSO-d₆) δ 10.31 (s, 1H), 8.78-8.79 (m, 1H), 8.55 (t, 1H), 8.31 (d, 1H), 8.15-8.16 (m, 1H), 8.11-8.12 (m, 1H), 7.80 (d, 1H), 7.45-7.50 (m, 3H), 7.37-7.41 (m, 2H), 7.31-7.35 (m, 1H), 5.31 (s, 1H), 3.70-3.71 (m, 2H), 3.48-3.49 (m, 2H), 2.58-2.60 (m, 1H), 2.46-2.49 (m, 2H), 2.32-2.36 (m, 1H), 2.11 (s, 3H).

### Example 85

### (1S,2S)-2-fluoro-N-(2-(2-(4-((5-methyl-2H-tetrazol-2-yl)(phenyl)methyl)piperidine-1-carbonyl)pyridin-4-yl)benzo[d]oxazol-5-yl)cyclopropanecarboxamide

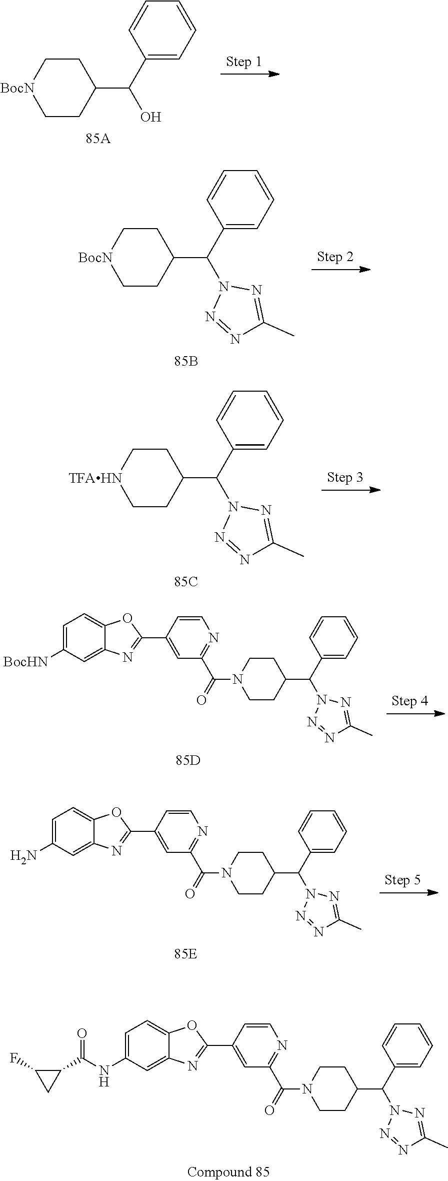

Step 1:

### tert-butyl 4-((5-methyl-2H-tetrazol-2-yl)(phenyl)methyl)piperidine-1-carboxylate (85B)

At room temperature, 85A (580 mg g, 2.0 mmol), 5-methyltetrazole (185 mg, 2.2 mmol) and triphenylphosphine (787 mg, 3.0 mmol) were dissolved in anhydrous THE (20 mL); the mixture was cooled to 0° C. under nitrogen protection; and then DEAD (520 mg, 3.0 mmol) was added dropwise. The mixture was allowed to naturally warm to room temperature, reacted overnight, concentrated under reduced pressure and subjected to column chromatography to obtain 85B (440 mg, 61.00%).

LC-MS (ESI): m/z=358.3[M+H]⁺.

Step 2:

### 4-((5-methyl-2H-tetrazol-2-yl)(phenyl)methyl)piperidin-1-ium 2,2,2-trifluoroacetate (85C)

85B (220 mg, 0.6 mmol) was dissolved in dichloromethane (10 mL) at room temperature; trifluoroacetic acid (2.5 mL) was added dropwise; and the mixture was stirred for another 2 hours; The reaction solution was subjected to rotary evaporation to obtain a crude product of 85C (300 mg), which was directly used in the next reaction without purification.

LC-MS (ESI): m/z=258.2[M+H]⁺.

Step 3:

### tert-butyl(2-(2-(4-((5-methyl-2H-tetrazol-2-yl)(phenyl)methyl)piperidine-1-carbonyl)pyridin-4-yl)benzo[d]oxazol-5-yl)carbamate (85D)

The crude product of 85C (300 mg) obtained in step 2 and 24A (219 mg, 0.6 mmol) were dissolved in DMF (10 mL) at room temperature; diisopropylethylamine (1.0 mL) was added dropwise with stirring; and finally, HATU (350 mg, 9.0 mmol) was added. After the addition, the mixture was reacted at room temperature overnight; 60 mL of ethyl acetate was added, and the mixture was washed with water (10 mL×3) and saturated sodium chloride solution (10 mL×1), dried over anhydrous sodium sulfate and concentrated under reduced pressure to obtain a crude product of 85D (250 mg), which was directly used in the next reaction without purification.

LC-MS (ESI): m/z=595.3[M+H]⁺.

Step 4:

### 2-(2-(4-((5-methyl-2H-tetrazol-2-yl)(phenyl)methyl)piperidine-1-carbonyl)pyridin-4-yl)benzo[d]oxazol-5-aminium 2,2,2-trifluoroacetate (85E)

85D (250 mg) was dissolved in dichloromethane (10 mL) at room temperature; trifluoroacetic acid (2.5 mL) was added dropwise; and the mixture was stirred for another 2 hours; The reaction solution was subjected to rotary evaporation to obtain a crude product of 85E (350 mg), which was directly used in the next reaction without purification.

LC-MS (ESI): m/z=495.3[M+H]⁺.

Step 5:

### (1S,2S)-2-fluoro-N-(2-(2-(4-((5-methyl-2H-tetrazol-2-yl)(phenyl)methyl)piperidine-1-carbonyl)pyridin-4-yl)benzo[d]oxazol-5-yl)cyclopropanecarboxamide (Compound 85)

The crude product of 85E (350 mg) obtained in step 4 and (1S,2S)-2-fluoro-clopropanecarboxylic acid (64 mg, 0.6 mmol) were dissolved in DMF (10 mL) at room temperature; diisopropylethylamine (1.0 mL) was added dropwise with stirring; and finally, HATU (350 mg, 9.0 mmol) was added. After the addition, the mixture was reacted at room temperature overnight. 60 mL of ethyl acetate was added; the reaction was washed with water (10 mL×3) and saturated sodium chloride solution (10 mL×1), dried over anhydrous sodium sulfate and concentrated under reduced pressure; and the residue was purified by column chromatography (PE:EA=10:1) to obtain compound 85.

LC-MS (ESI): m/z=581.3[M+H]⁺.

¹H NMR (400 MHz, CDCl₃) δ 8.74-8.71 (m, 1H), 8.28-8.25 (m, 1H), 8.10-7.98 (m, 3H), 7.56-7.48 (m, 4H), 7.43-7.31 (m, 3H), 5.55-5.52 (m, 1H), 4.91-4.75 (m, 2H), 3.91-3.88 (m, 1H), 3.19-3.11 (m, 1H), 2.91-2.80 (m, 2H), 2.56-2.50 (m, 3H), 2.15-2.05 (m, 1H), 1.94-1.84 (m, 2H), 1.61-1.23 (m, 4H).

### Example 86

### N-(2-(2-(4-(3-cyclopropyl-1-(2-methyl-2H-tetrazol-5-yl)prop-2-yn-1-yl)piperazine-1-carbonyl)pyridin-4-yl)benzo[d]oxazol-5-yl)acetamide

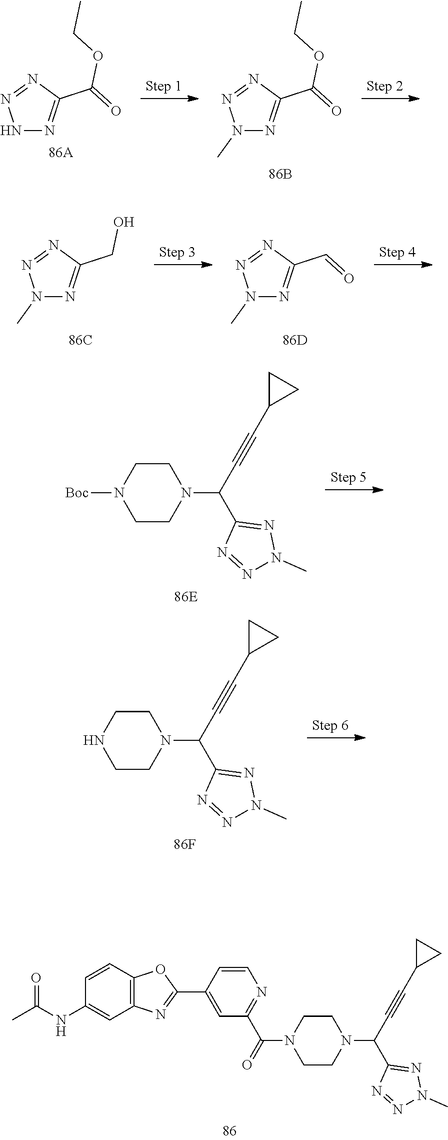

Step 1:

### Ethyl 2-methyl-2H-tetrazole-5-carboxylate (86B)

To a 100 ml round bottom flask, ethyl 2H-tetrazole-5-carboxylate (5.08 g, 35.18 mmol) was added and dissolved in a mixed solvent of THF/MeOH (4:1), and then TMSCHN₂ (trimethylsilazodimethane) (26 ml, 52.0 mmol) was added, and the mixture was reacted at room temperature for 2 h. The reaction solution was concentrated by column chromatography (eluent: PE:EA=5:1) to obtain compound 86B (2.05 g, 36%).

LC-MS (ESI): m/z=157.1 [M+H]⁺.

Step 2:

### (2-methyl-2H-tetrazol-5-yl)methanol (86C)

To a 100 ml round bottom flask, ethyl 2-methyl-2H-tetrazole-5-carboxylate (2.05 g, 12.81 mmol) was added and dissolved in MeOH, and then NaBH₄ (0.82 g, 51.24 mmol) was added. The mixture was stirred at room temperature for 3 h. Saturated ammonium chloride solution was added, and the aqueous phase was extracted with DCM. The organic phase was collected, washed with saturated brine, collected, dried over anhydrous sodium sulfate and concentrated to obtain compound 86C (1.03 g, 68%).

LC-MS (ESI): m/z=115.1 [M+H]⁺.

Step 3:

### 2-methyl-2H-tetrazole-5-carbaldehyde (86D)

To a 100 ml round bottom flask, (2-methyl-2H-tetrazol-5-yl)methanol (1.03 g, 8.76 mmol) was added and dissolved in acetonitrile; IBX (9.82 g, 35.05 mmol) was added; and the mixture was reacted at 60° C. for 4 h. The reaction solution was filtered by suction. The filtrate was concentrated, and the residue was purified by preparative thin-layer chromatography (eluent: PE:EA=5:1) to obtain compound 86D (725 mg, 74%).

LC-MS (ESI): m/z=113.1 [M+H]⁺.

Step 4:

### tert-butyl-4-(3-cyclopropyl-1-(2-methyl-2H-tetrazol-5-yl)prop-2-yn-1-yl)piperazine-1-carboxylate (86E)

To a 100 ml round bottom flask, 2-methyl-2H-tetrazole-5-carbaldehyde (0.50 g, 4.46 mmol) was added and mixed evenly with acetonitrile; tert-butyl piperazine-1-carboxylate (0.85 g, 4.55 mmol), cyclopropyl acetylene (0.27 g, 4.01 mmol) and CuI (0.17 g, 0.89 mmol) were added; and the mixture was refluxed and reacted for 6 h. The reaction solution was concentrated. The crude product (86E) was used directly in the next step.

LC-MS (ESI): m/z=347.2 [M+H]⁺.

Step 5:

### 1-(3-cyclopropyl-1-(2-methyl-2H-tetrazol-5-yl)prop-2-yn-1-yl)piperazine (86F)

To a 50 ml round bottom flask, the crude product of tert-butyl-4-(3-cyclopropyl-1-(2-methyl-2H-tetrazol-5-yl)prop-2-yn-1-yl)piperazine-1-carboxylate was added and dissolved in a mixed solvent of DCM:TFA (2:1) (12 ml). The mixture was reacted at room temperature for 3 h. DCM was added, and the resulting solution was extracted once. The aqueous phase was collected, adjusted to pH=9-10 by adding 20% NaOH, extracted twice with DCM and washed with saturated brine. The organic phase was collected, dried over anhydrous sodium sulfate and concentrated. A second batch of organic phase was concentrated and subjected to rotary evaporation. The crude product (500 mg, 45%) was obtained.

LC-MS (ESI): m/z=247.2 [M+H]⁺.

Step 6:

### N-(2-(2-(4-(3-cyclopropyl-1-(2-methyl-2H-tetrazol-5-yl)prop-2-yn-1-yl)piperazine-1-carbonyl)pyridin-4-yl)benzo[d]oxazol-5-yl)acetamide (Compound 86)

To a 100 ml round bottom flask, 1-(3-cyclopropyl-1-(2-methyl-2H-tetrazol-5-yl)prop-2-yn-1-yl)piperazine (0.50 g, 2.03 mmol) was added and dissolved in DMF. Intermediate 1 (0.60 g, 2.03 mmol), DIEA (0.79 g, 6.09 mmol) and HATU (1.16 g, 3.04 mmol) were added. The mixture was reacted at room temperature for 2 h. Water was added; the aqueous phase was extracted with ethyl acetate; the organic phase was collected, washed with saturated brine, dried over anhydrous sodium sulfate and concentrated; and the residue was subjected to prep-HPLC to obtain compound 86 (147 mg, 14%). Preparation conditions: instrument: waters 2767 (preparative liquid phase chromatographic instrument); chromatographic column: XBridge C18 5 μm, 19×250 mm. The sample was dissolved in acetonitrile and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 5% to 50%; time: 15 min; flow rate: 12 ml/min. Components with retention time of 10.5 min were used.

LC-MS (ESI): m/z=526.2 [M+H]⁺.

¹H NMR (400 MHz, DMSO-d₆) δ 10.16 (s, 1H), 8.84 (d, 1H), 8.23-8.14 (m, 3H), 7.78 (d, 1H), 7.59 (d, 1H), 5.13 (s, 1H), 4.36 (s, 3H), 3.71-3.57 (m, 2H), 3.48-3.46 (m, 2H), 2.67-2.53 (m, 4H), 2.09 (s, 3H), 1.41-1.38 (m, 1H), 0.84-0.80 (m, 2H), 0.65-0.61 (m, 2H).

### Example 87

### N-(2-(2-(4-((2-(difluoromethyl)-2H-tetrazol-5-yl)(thiophen-3-yl)methyl)piperazine-1-carbonyl)pyridin-4-yl)benzo[d]oxazol-5-yl)acetamide

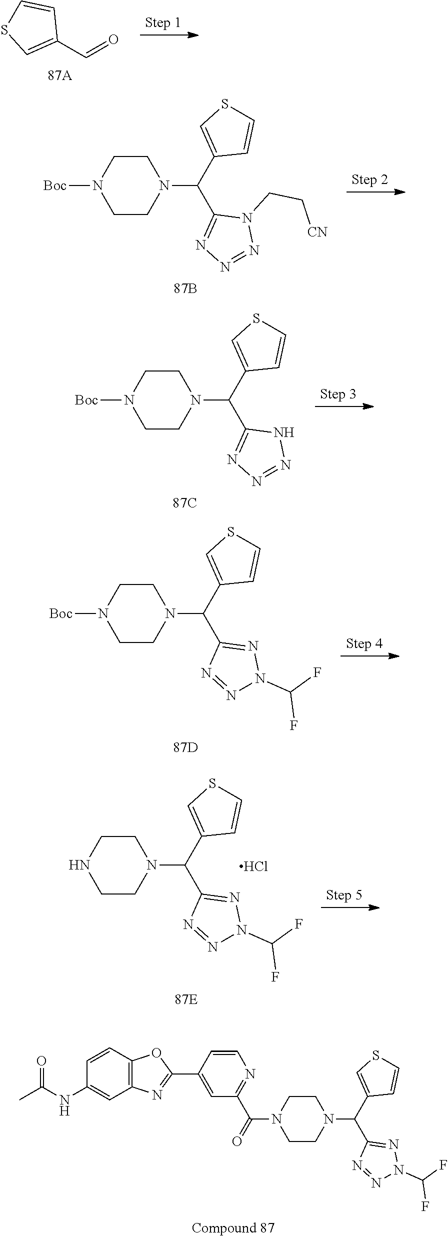

Step 1:

### tert-butyl 4-((1-(2-cyanoethyl)-1H-tetrazol-5-yl)(thiophen-3-yl)methyl)piperazine-1-carboxylate (87B)

Compound 87A (7.0 g, 62.5 mmol) was dissolved in methanol (100 mL). 1-tert-butoxycarbonyl piperazine (11.6 g, 62.5 mmol) was added at room temperature. After the mixture was reacted for ten minutes, azidotrimethylsilane (7.2 g, 62.5 mmol) and 3-isocyanopropanenitrile (5.0 g, 62.5 mmol) were successively added. The reaction solution was stirred at 35° C. for 8 hours and filtered. The filter cake was washed once with 30 mL of methanol and dried under reduced pressure to obtain 87B (12 g, 48%).

LC-MS (ESI): m/z=404.2 [M+H]⁺.

Step 2:

### tert-butyl 4-((1H-tetrazol-5-yl)(thiophen-3-yl)methyl)piperazine-1-carboxylate (87C)

At room temperature, compound 87B (6.0 g, 14.9 mmol) was dissolved in tetrahydrofuran (50 mL), and lithium hydroxide (1.8 g, 74.5 mmol) was dissolved in 50 mL of pure water. An aqueous solution of lithium hydroxide was added to the reaction solution; the mixture was stirred at room temperature for 2 hours and then adjusted to pH 6-7 with 2 N hydrochloric acid; and the resulting solution was extracted with ethyl acetate (100 mL×3). The combined organic phase was dried over anhydrous sodium sulfate and concentrated under reduced pressure to obtain 87C (5 g, 96%).

LC-MS (ESI): m/z=351.2 [M+H]⁺.

Step 3:

### tert-butyl 4-((2-(difluoromethyl)-2H-tetrazol-5-yl)(thiophen-3-yl)methyl)piperazine-1-carboxylate (87D)

Compound 87C (5.0 g, 14.3 mmol) was dissolved in acetonitrile (60 mL); pure water (15 mL) was added; and then potassium hydroxide (1.2 g, 21.5 mmol) was added. The reaction was stirred at 0° C. for 10 minutes, and diethyl(bromodifluoromethyl)phosphonate (3.8 g, 14.3 mmol) was added quickly. The reaction solution was stirred at 0° C. for 30 minutes. Water (100 mL) was added to the reaction solution, and the resulting solution was extracted with ethyl acetate (100 mL×3). The combined organic phase was dried over anhydrous sodium sulfate and concentrated under reduced pressure, and the residue was separated and purified by silica gel column chromatography (EA:PE=20%-40%) to obtain 87D (2.2 g, 39%).

LC-MS (ESI): m/z=401.1 [M+H]⁺.

Step 4:

### 1-((2-(difluoromethyl)-2H-tetrazol-5-yl)(thiophen-3-yl)methyl)piperazine Hydrochloride (87E)

A solution of hydrochloric acid in dioxane (20 mL) was added to compound 87D (2.2 g, 5.5 mmol) at room temperature, and the mixture was stirred for 1 h and then concentrated under reduced pressure to obtain 87E (1.8 g, 97%).

LC-MS (ESI): m/z=301.1 [M+H]⁺.

Step 5:

### N-(2-(2-(4-((2-(difluoromethyl)-2H-tetrazol-5-yl)(thiophen-3-yl)methyl)piperazine-1-carbonyl)pyridin-4-yl)benzo[d]oxazol-5-yl)acetamide (Compound 87)

DMF (20 mL), 4-(5-acetamidobenzo[d]oxazol-2-yl)picolinic acid (442 mg, 1.49 mmol), HATU (1.1 g, 3 mmol) and DIEA (451 mg, 3.5 mmol) were successively added to compound 87E (500 mg, 1.49 mmol), and the mixture was stirred at room temperature for 5 h. The reaction solution was extracted with ethyl acetate and water and washed twice with saturated brine. The organic phase was dried and concentrated to obtain a crude product compound, which was subjected to preparative chromatography to obtain compound 87 (200 mg, 23%). Preparative separation conditions: instrument: waters 2767 (preparative liquid phase chromatographic instrument); chromatographic column: XBridge@ Prep C18 (19 mm×250 mm); The sample was dissolved in DMF and filtered with a 0.45 m filter to prepare a sample solution. Preparative chromatography conditions: mobile phase A: acetonitrile; mobile phase B: water (containing 0.5% ammonia water); gradient elution: the content of mobile phase A rises from 50% to 75%; flow rate: 15 ml/min; elution time: 20 min; retention time: about 16 min;

¹H NMR (400 MHz, DMSO-d₆) δ 10.17 (s, 1H), 8.81 (d, 1H), 8.42-8.72 (m, 1H), 8.05-8.28 (m, 3H), 7.78 (d, 1H), 7.55-7.65 (m, 3H), 7.21 (d, 1H), 5.63 (s, 1H), 3.74 (s, 2H), 3.53 (s, 2H), 2.41-2.78 (m, 4H), 2.09 (s, 3H).

LC-MS (ESI): m/z=580 [M+H]⁻.

### Example 88

### (R/S)-(4-((2-(difluoromethyl)-2H-tetrazol-5-yl)(phenyl)methyl)piperazin-1-yl)(4-(5-(1-methyl-1H-imidazol-2-yl)benzo[d]oxazol-2-yl)pyridin-2-yl)methanone

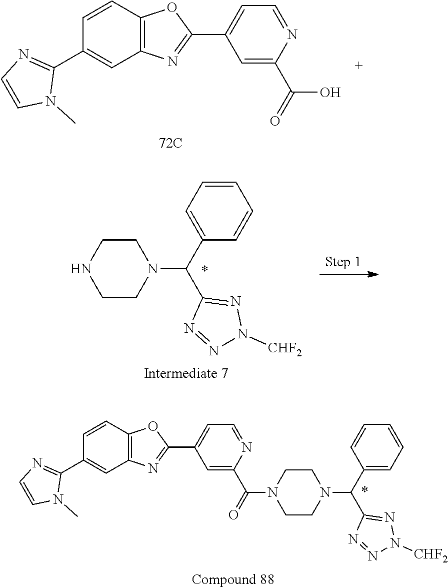 [SMILES: Cn1ccnc1-c1ccc2oc(-c3ccnc(C(=O)O)c3)nc2c1 ; FC(F)n1nnc(C(c2ccccc2)N2CCNCC2)n1 ; Cn1ccnc1-c1ccc2oc(-c3ccnc(C(=O)N4CCN(C(c5ccccc5)c5nnn(C(F)F)n5)CC4)c3)nc2c1]

At room temperature, DMF (8 mL), intermediate 7 (148 mg, 0.51 mmol), HATU (291 mg, 0.77 mmol) and DIPEA (197 mg, 1.53 mmol) were successively added to compound 72C (163 mg, 0.51 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 88 (50 mg, 16%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.98 min.

LC-MS (ESI): m/z=597.3 [M+H]⁺.

¹H NMR (400 MHz, DMSO-d₆) δ8.86-8.87 (m, 1H), 8.56 (t, 1H), 8.41-8.42 (m, 1H), 8.25-8.26 (m, 1H), 8.18-8.21 (m, 2H), 7.91-7.93 (m, 1H), 7.85-7.89 (m, 2H), 7.49-7.51 (m, 2H), 7.32-7.41 (m, 3H), 5.34 (s, 1H), 3.91 (s, 3H), 3.72-3.73 (m, 2H), 3.52-3.53 (m, 2H), 2.60-2.62 (m, 1H), 2.52-2.56 (m, 2H), 2.36-2.39 (m, 1H).

### Example 89

### (R/S)-2-(2-(4-((2-(difluoromethyl)-2H-tetrazol-5-yl)(phenyl)methyl)piperazine-1-carbonyl)pyridin-4-yl)benzo[d]oxazole-5-carbonitrile

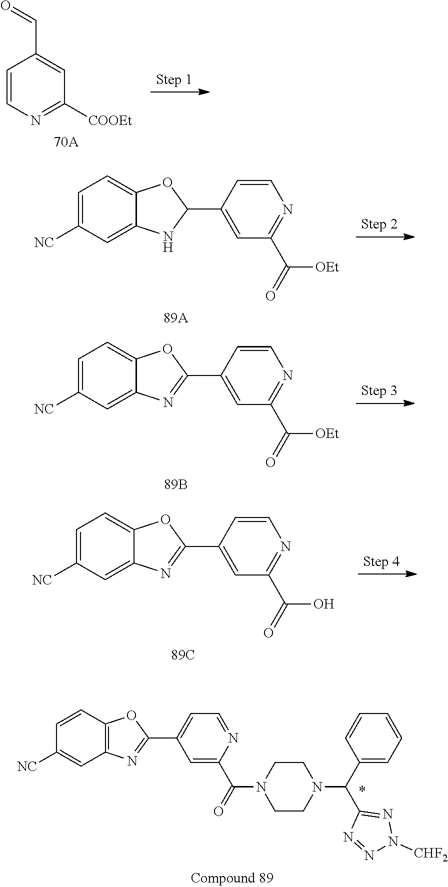 [SMILES: CCOC(=O)c1cc(C=O)ccn1 ; CCOC(=O)c1cc(C2Nc3cc(C#N)ccc3O2)ccn1 ; CCOC(=O)c1cc(-c2nc3cc(C#N)ccc3o2)ccn1 ; N#Cc1ccc2oc(-c3ccnc(C(=O)O)c3)nc2c1 ; N#Cc1ccc2oc(-c3ccnc(C(=O)N4CCN(C(c5ccccc5)c5nnn(C(F)F)n5)CC4)c3)nc2c1]

Step 1:

### Ethyl 4-(5-cyano-2,3-dihydrobenzo[d]oxazol-2-yl)picolinate (89A)

Compound 70A (1.0 g, 5.59 mmol) and 3-amino-4-hydroxybenzonitrile (0.749 g, 5.59 mmol) were added to ethanol (20 mL), and the mixture was warmed to 75° C. and stirred for 5 h. The reaction was cooled to room temperature and concentrated under reduced pressure to remove ethanol, and 89A (1.64 g, 99%) was obtained.

Step 2:

### Ethyl 4-(5-cyanobenzo[d]oxazol-2-yl)picolinate (89B)

Compound 89A (1.64 g, 5.59 mmol) was added to dichloromethane (80 mL), and then manganese dioxide (2.43 g, 27.95 mmol) was added. The mixture was stirred at 25° C. for 10 hours. The resulting solution was filtered; the filtrate was concentrated; and then the residue was separated and purified by silica gel column chromatography (PE:EA=1:1) to obtain 89B (1.3 g, 79%).

LC-MS (ESI): m/z=294.1[M+H]⁺.

Step 3:

### 4-(5-cyanobenzo[d]oxazol-2-yl)picolinic Acid (89C)

Anhydrous methanol (10 mL) and NaOH (170 mg, 4.27 mmol, 1 mL) aqueous solution were successively added to compound 89B (250 mg, 0.85 mmol). The mixture was stirred at room temperature overnight, concentrated under reduced pressure to remove most of methanol, adjusted to pH=2-3 by dropwise adding 1 N hydrochloric acid and filtered. The filter cake was washed with water (2 mL) and dried to obtain compound 89C (200 mg, 89%).

LC-MS (ESI): m/z=266.1 [M+H]⁺.

Step 4:

### (R)-2-(2-(4-((2-(difluoromethyl)-2H-tetrazol-5-yl)(phenyl)methyl)piperazine-1-carbonyl)pyridin-4-yl)benzo[d]oxazole-5-carbonitrile

At room temperature, DMF (8 mL), intermediate 7 (144 mg, 0.49 mmol), HATU (279 mg, 0.74 mmol) and DIPEA (190 mg, 1.47 mmol) were successively added to compound 89C (130 mg, 0.49 mmol). The reaction was stirred for 1 hour. The reaction solution was poured into water (50 mL) and extracted with ethyl acetate (20 mL×3). The organic phase was concentrated, and the residue was separated and purified by HPLC to obtain compound 89 (50 mg, 19%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.13 min.

LC-MS (ESI): m/z=542.2 [M+H]⁺.

¹H NMR (400 MHz, DMSO-d₆) δ8.84-8.85 (m, 1H), 8.55 (t, 1H), 8.53-8.54 (m, 1H), 8.22-8.23 (m, 1H), 8.17-8.19 (m, 1H), 8.09 (d, 1H), 7.98-8.00 (m, 1H), 7.48-7.50 (m, 2H), 7.37-7.41 (m, 2H), 7.31-7.35 (m, 1H), 5.32 (s, 1H), 3.72-3.73 (m, 2H), 3.49-3.50 (m, 2H), 2.58-2.61 (m, 1H), 2.47-2.51 (m, 2H), 2.33-2.37 (m, 1H).

### Example 90

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-6-yl)acetamide

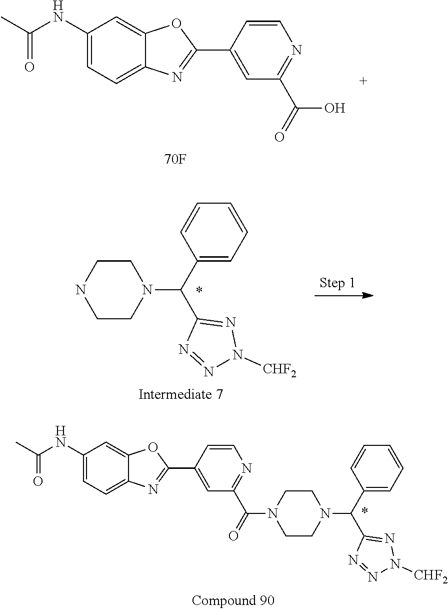 [SMILES: CC(=O)Nc1ccc2nc(-c3ccnc(C(=O)O)c3)oc2c1 ; FC(F)n1nnc(C(c2ccccc2)N2CCNCC2)n1 ; CC(=O)Nc1ccc2nc(-c3ccnc(C(=O)N4CCN(C(c5ccccc5)c5nnn(C(F)F)n5)CC4)c3)oc2c1]

At room temperature, DMF (8 mL), intermediate 7 (148 mg, 0.51 mmol), HATU (291 mg, 0.77 mmol) and DIPEA (197 mg, 1.53 mmol) were successively added to compound 70F (150 mg, 0.51 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 90 (30 mg, 10%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.59 min.

LC-MS (ESI): m/z=574.3 [M+H]⁺.

¹H NMR (400 MHz, DMSO-d₆) δ 10.31 (s, 1H), 8.78-8.79 (m, 1H), 8.55 (t, 1H), 8.31-8.32 (m, 1H), 8.15-8.16 (m, 1H), 8.11-8.12 (m, 1H), 7.80 (d, 1H), 7.45-7.50 (m, 3H), 7.37-7.41 (m, 2H), 7.31-7.35 (m, 1H), 5.31 (s, 1H), 3.70-3.71 (m, 2H), 3.48-3.49 (m, 2H), 2.58-2.60 (m, 1H), 2.47-2.49 (m, 2H), 2.32-2.36 (m, 1H), 2.11 (s, 3H).

### Example 91

### (R)-2,2-difluoro-N-(2-(2-(4-((R)-(2-methyl-2H-tetrazol-5-yl)(phenyl)methyl)piperazine-1-carbonyl)pyridin-4-yl)benzo[d]oxazol-5-yl)cyclopropane-1-carboxamide (Compound 91)

### Example 92

### (R)-2,2-difluoro-N-(2-(2-(4-((S)-(2-methyl-2H-tetrazol-5-yl)(phenyl)methyl)piperazine-1-carbonyl)pyridin-4-yl)benzo[d]oxazol-5-yl)cyclopropane-1-carboxamide (Compound 92)

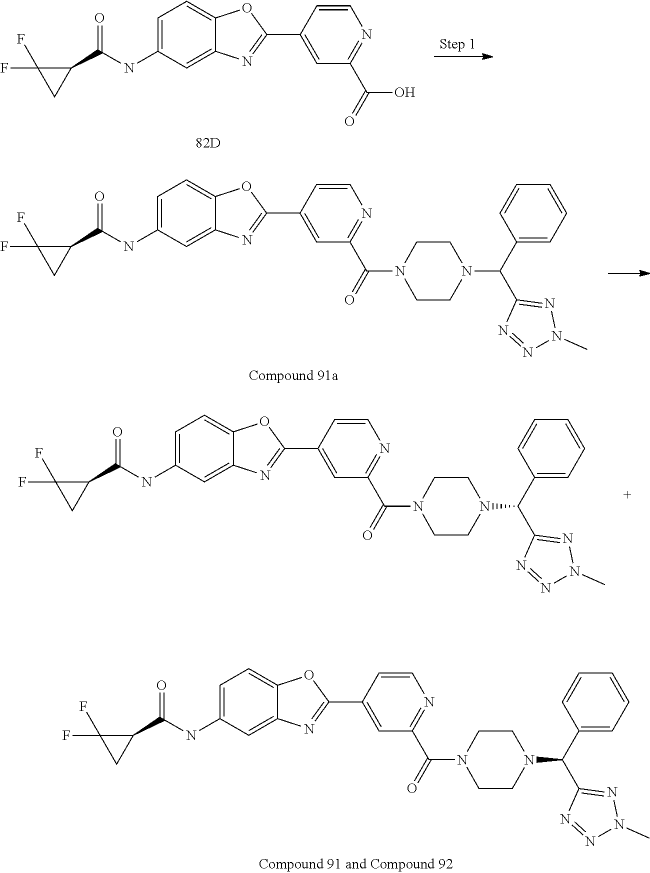 [SMILES: O=C(O)c1cc(-c2nc3cc(NC(=O)[C@H]4CC4(F)F)ccc3o2)ccn1 ; Cn1nnc(C(c2ccccc2)N2CCN(C(=O)c3cc(-c4nc5cc(NC(=O)[C@H]6CC6(F)F)ccc5o4)ccn3)CC2)n1 ; Cn1nnc([C@@H](c2ccccc2)N2CCN(C(=O)c3cc(-c4nc5cc(NC(=O)[C@H]6CC6(F)F)ccc5o4)ccn3)CC2)n1 ; Cn1nnc([C@H](c2ccccc2)N2CCN(C(=O)c3cc(-c4nc5cc(NC(=O)[C@H]6CC6(F)F)ccc5o4)ccn3)CC2)n1]

At room temperature, DMF (15 mL), intermediate 2 (216 mg, 0.84 mmol), HATU (479 mg, 1.26 mmol) and DIPEA (325 mg, 2.52 mmol) were successively added to compound 82D (300 mg, 0.84 mmol). The reaction was stirred for 1 hour. The reaction solution was poured into water (50 mL) and extracted with ethyl acetate (20 mL×3). The organic phase was concentrated, and the residue was separated and purified by silica gel column chromatography (DCM:MeOH=30:1) to obtain compound 91a (300 mg). 91a was purified and separated by chiral HPLC (instrument name: MG II preparative SFC (SFC-1); chromatographic column: Whelk O1(S, S), 250×30 mm I.D., 5 μm; mobile phase: A for CO₂ and B for Methanol; gradient: B 40%; flow rate: 70 mL/min; column pressure: 100 bar; column temperature: 38° C.; absorption wavelength: 220 nm; cycle time: about 30 min) to obtain compound 91 (58 mg) and compound 92 (71 mg). retention time for compound 91: 17.62 min; retention time for compound 92: 23.06 min. Compounds 91 and 92 were compounds in single configurations.

Compound 91 LC-MS (ESI): m/z=600.3 [M+H]⁺.

¹H NMR (400 MHz, DMSO-d₆) δ 10.66 (s, 1H), 8.80-8.81 (m, 1H), 8.21-8.22 (m, 1H), 8.17-8.18 (m, 1H), 8.13-8.15 (m, 1H), 7.82 (d, 1H), 7.59-7.62 (m, 1H), 7.48-7.50 (m, 2H), 7.34-7.37 (m, 2H), 7.27-7.31 (m, 1H), 5.10 (s, 1H), 4.35 (s, 3H), 3.68-3.71 (m, 2H), 3.44-3.47 (m, 2H), 2.80-2.88 (m, 1H), 2.51-2.53 (m, 1H), 2.45-2.51 (m, 2H), 2.30-2.33 (m, 1H), 2.01-2.09 (m, 2H).

Compound 92 LC-MS (ESI): m/z=600.3 [M+H]⁺.

¹H NMR (400 MHz, DMSO-d₆) δ 10.66 (s, 1H), 8.80-8.81 (m, 1H), 8.21-8.22 (m, 1H), 8.17-8.18 (m, 1H), 8.13-8.15 (m, 1H), 7.82 (d, 1H), 7.59-7.62 (m, 1H), 7.48-7.50 (m, 2H), 7.34-7.37 (m, 2H), 7.27-7.31 (m, 1H), 5.10 (s, 1H), 4.35 (s, 3H), 3.68-3.71 (m, 2H), 3.44-3.47 (m, 2H), 2.80-2.88 (m, 1H), 2.51-2.53 (m, 1H), 2.45-2.51 (m, 2H), 2.30-2.33 (m, 1H), 2.01-2.09 (m, 2H).

### Example 93

### (1S,2S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-6-yl)-2-fluorocyclopropane-1-carboxamide

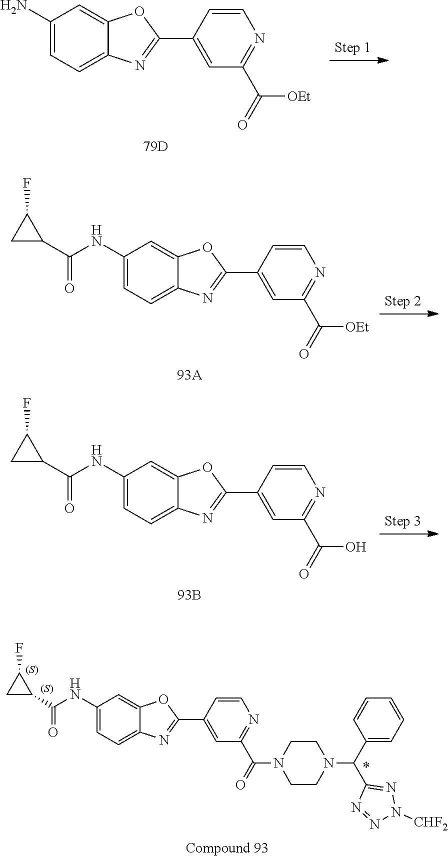

Step 1:

### Ethyl 4-(6-((1 S,2S)-2-fluorocyclopropane-1-carboxamido)benzo[d]oxazol-2-yl)picolinate (93A)

At room temperature, DMF (15 mL), (1S,2S)-2-fluorocyclopropane-1-carboxylic acid (147 mg, 1.41 mmol), HATU (804 mg, 2.12 mmol) and DIPEA (546 mg, 4.23 mmol) were successively added to compound 70D (400 mg, 1.41 mmol). The reaction was stirred for 1 hour. The reaction solution was poured into water (50 mL) and extracted with ethyl acetate (20 mL×3). The organic phase was concentrated, and the residue was separated and purified by silica gel column chromatography (PE:EA=1:5) to obtain compound 93A (450 mg, 87%).

LC-MS (ESI): m/z=370.1 [M+H]⁺.

Step 2:

### 4-(6-((1S,2S)-2-fluorocyclopropane-1-carboxamido)benzo[d]oxazol-2-yl)picolinic Acid (93B)

Anhydrous methanol (10 mL), water (2 mL) and NaOH (244 mg, 6.10 mmol) were successively added to compound 93A (0.45 g, 1.22 mmol). The mixture was stirred at room temperature for 10 h, concentrated under reduced pressure to remove most of methanol, adjusted to pH=2-3 by dropwise adding 1 N hydrochloric acid and filtered. The filter cake was washed with water (3 mL) and dried to obtain intermediate 93B (330 mg, 79%).

LC-MS (ESI): m/z=342.1 [M+H]⁺.

Step 3:

### (1S,2S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-6-yl)-2-fluorocyclopropane-1-carboxamide (Compound 93)

At room temperature, DMF (5 mL), intermediate 7 (112 mg, 0.28 mmol), HATU (217 mg, 0.57 mmol) and DIPEA (221 mg, 1.71 mmol) were successively added to compound 93B (130 mg, 0.38 mmol). The reaction was stirred for 1 hour. The reaction solution was poured into water (50 mL) and extracted with ethyl acetate (20 mL×3). The organic phase was concentrated, and the residue was separated and purified by HPLC to obtain compound 93 (40 mg, 17%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.04 min.

LC-MS (ESI): m/z=618.3 [M+H]⁺.

¹H NMR (400 MHz, DMSO-d₆) δ 10.62 (s, 1H), 8.78-8.79 (m, 1H), 8.55 (t, 1H), 8.31-8.32 (m, 1H), 8.15-8.16 (m, 1H), 8.11-8.13 (m, 1H), 7.82 (d, 1H), 7.48-7.52 (m, 3H), 7.37-7.41 (m, 2H), 7.31-7.35 (m, 1H), 5.32 (s, 1H), 5.03-5.07 (m, 0.5H), 4.86-4.90 (m, 0.5H), 3.70-3.71 (m, 2H), 3.48-3.49 (m, 2H), 2.58-2.61 (m, 1H), 2.45-2.49 (m, 2H), 2.33-2.36 (m, 1H), 2.03-2.10 (m, 1H), 1.65-1.73 (m, 1H), 1.14-1.23 (m, 1H).

### Example 94

### (S/R)—N-(2-(2-(4-((R)-(2-(difluoromethyl)-2H-tetrazol-5-yl)(phenyl)methyl)piperazine-1-carbonyl)pyridin-4-yl)benzo[d]oxazol-5-yl)oxetane-2-carboxamide

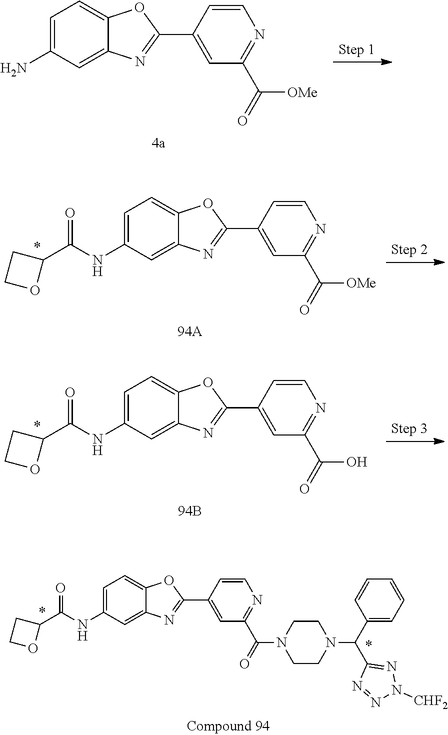 [SMILES: COC(=O)c1cc(-c2nc3cc(N)ccc3o2)ccn1 ; COC(=O)c1cc(-c2nc3cc(NC(=O)C4CCO4)ccc3o2)ccn1 ; O=C(O)c1cc(-c2nc3cc(NC(=O)C4CCO4)ccc3o2)ccn1 ; O=C(Nc1ccc2oc(-c3ccnc(C(=O)N4CCN(C(c5ccccc5)c5nnn(C(F)F)n5)CC4)c3)nc2c1)C1CCO1]

Step 1:

### Methyl (S)-4-(5-(oxetane-2-carboxamido)benzo[d]oxazol-2-yl)picolinate (94A)

Compound 4a (1.3 g, 4.88 mmol) was dissolved in N,N-dimethylformamide (15 mL), and the mixture was cooled to 0° C.; HATU (2.7 g, 7.32 mmol) and DIPEA (1.9 g, 14.64 mmol) were added; and then 2-oxetanecarboxylic acid (547.7 mg, 5.37 mmol) was added. After the addition, the mixture was stirred for 1 hour while the temperature was controlled at 0° C., and then the reaction was quenched by adding ice water (10 mL). The resulting solution was extracted twice with dichloromethane (50 mL×2). The organic phase was combined and dried over anhydrous sodium sulfate. Compound 94A (1.2 g, 70%) was obtained by SFC chiral preparative separation.

Preparation method: (instrument name: MG II preparative SFC (SFC-1); chromatographic column: ChiralPak AD, 250×30 mm I.D., 10 μm; mobile phase: A for CO₂ and B for isopropanol; gradient: B 50%; flow rate: 80 mL/min; column pressure: 100 bar; column temperature: 38° C.; absorption wavelength: 220 nm; cycle time: about 3 min). retention time for compound 94A: 4.320 min. Compound 94A is a compound in a single configuration.

LC-MS (ESI): m/z=354.2 [M+H]⁺.

Step 2:

### (S)-4-(5-(oxetane-2-carboxamido)benzo[d]oxazol-2-yl)picolinic Acid (94B)

Methanol (5 mL) and sodium hydroxide (68 mg, 1.70 mmol) were successively added to compound 94A (400 mg, 1.13 mmol), and the reaction was stirred at room temperature for 2 hours. The solvent was subjected to rotary evaporation, and the residue was adjusted to pH=4-5 by dropwise adding 1 M hydrochloric acid solution. The solid was rinsed with a small amount of water and concentrated under reduced pressure to obtain compound 94B (337 mg, 88%).

LC-MS (ESI): m/z=340.3[M+H]⁺.

Step 3:

### (2S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)oxetane-2-carboxamide (Compound 94)

Compound 94B (168 mg, 0.50 mmol) was dissolved in N,N-dimethylformamide (5 mL). HATU (285.1 mg, 0.75 mmol) and DIPEA (193.5 mg, 1.50 mmol) were successively added, and then intermediate 7 (176.4 mg, 0.60 mmol) was added. The mixture was reacted at room temperature for one hour. The reaction solution was poured into water (50 mL) and extracted with ethyl acetate (20 mL×2). The organic phase was concentrated, and the residue was separated and purified by preparative HPLC to obtain compound 94 (24 mg, 7.8%). instrument: waters 2767 (preparative liquid phase chromatographic instrument); chromatographic column: SunFire@ Prep C18 (19 mm×250 mm; The sample was dissolved in DMF and filtered with a 0.45 m filter to prepare a sample solution. Preparative chromatography conditions: a. mobile phases A and B: mobile phase A: acetonitrile; mobile phase B: water (containing 5 mM ammonium acetate); b. gradient elution: the content of mobile phase A rises from 40% to 70%; c. flow rate: 15 mL/min; d. elution time: 20 min; retention time: 11.15 min.

LC-MS (ESI): m/z=616.6 [M+H]⁺.

1H NMR (400 MHz, Chloroform-d) δ 9.68 (s, 1H), 9.07 (d, 1H), 8.75 (d, 1H), 8.43 (d, 1H), 8.26 (dd, 1H), 7.64 (d, 1H), 7.61-7.54 (m, 2H), 7.51 (dd, 1H), 7.33-7.23 (m, 3H), 4.99 (t, 1H), 4.49 (dd, 1H), 3.81-3.68 (m, 2H), 3.62-3.47 (m, 4H), 2.61 (t, 4H), 2.24-2.08 (m, 2H).

### Example 95

### (1R,2S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-5-yl)-2-fluorocyclopropane-1-carboxamide

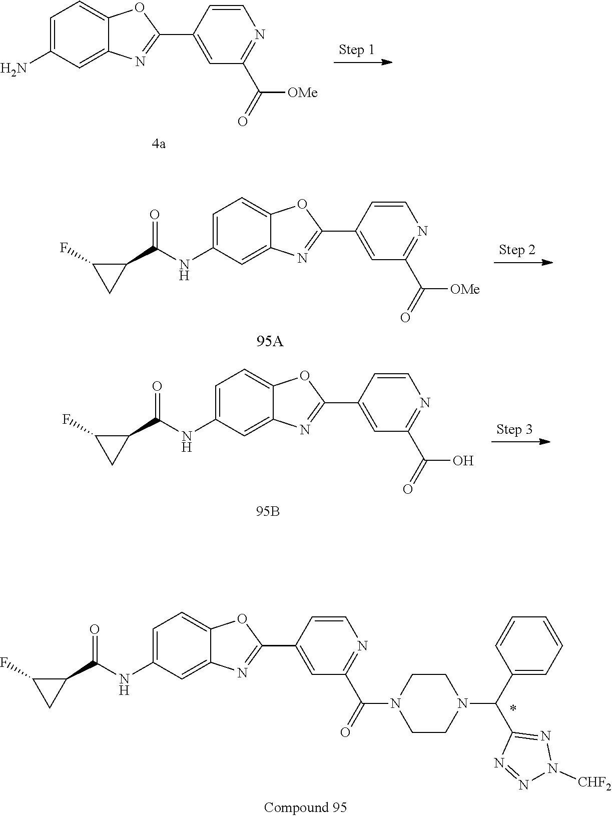 [SMILES: COC(=O)c1cc(-c2nc3cc(N)ccc3o2)ccn1 ; COC(=O)c1cc(-c2nc3cc(NC(=O)[C@H]4C[C@@H]4F)ccc3o2)ccn1 ; O=C(O)c1cc(-c2nc3cc(NC(=O)[C@H]4C[C@@H]4F)ccc3o2)ccn1 ; O=C(Nc1ccc2oc(-c3ccnc(C(=O)N4CCN(C(c5ccccc5)c5nnn(C(F)F)n5)CC4)c3)nc2c1)[C@H]1C[C@@H]1F]

Step 1:

### Methyl 4-(5-((1R,2S)-2-fluorocyclopropane-1-carboxamido)benzo[d]oxazol-2-yl)picolinate (95A)

Compound 4a (550 mg, 2.04 mmol) was dissolved in N,N-dimethylformamide (2 mL), and the mixture was cooled to 0° C.; HATU (1.01 g, 2.66 mmol) and DIPEA (789.5 mg, 6.12 mmol) were added; and then (1R,2S)-2-fluorocyclopropane-1-carboxylic acid (254.6 mg, 2.45 mmol) was added. After the addition, the mixture was stirred for 1 hour while the temperature was controlled at 0° C., and then the reaction was quenched by adding ice water (10 mL). The resulting solution was extracted twice with dichloromethane (50 mL×2). The organic phase was combined and dried over anhydrous sodium sulfate. The reaction solution was separated by column chromatography (PE:EA=1:1) to obtain compound 95A (674.5 mg, 93%).

LC-MS (ESI): m/z=356.3 [M+H]⁺.

Step 2:

### 4-(5-((1R,2S)-2-fluorocyclopropane-1-carboxamido)benzo[d]oxazol-2-yl)picolinic Acid (95B)

Methanol (5 mL) and sodium hydroxide (152 mg, 3.8 mmol) were successively added to compound 95A (674.5 mg, 1.9 mmol), and the reaction was stirred at room temperature for 2 hours. The solvent was subjected to rotary evaporation, and the residue was adjusted to pH=4-5 by dropwise adding 1 M hydrochloric acid solution. The solid was rinsed with a small amount of water and concentrated under reduced pressure to obtain compound 95B (590 mg, 91%).

LC-MS (ESI): m/z=342.3[M+H]⁺.

Step 3:

### (1R,2S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-5-yl)-2-fluorocyclopropane-1-carboxamide (Compound 95)

Compound 95B (68 mg, 0.11 mmol) was dissolved in N,N-dimethylformamide (2 mL). HATU (63.6 mg, 0.17 mmol) and DIPEA (14 mg, 0.33 mmol) were successively added, and then intermediate 7 (38.8 mg, 0.13 mmol) was added. The mixture was reacted at room temperature for one hour. The reaction solution was poured into water (50 mL) and extracted with ethyl acetate (20 mL×2). The organic phase was concentrated, and the residue was separated and purified by preparative HPLC to obtain compound 95 (25 mg, 37%). instrument: waters 2767 (preparative liquid phase chromatographic instrument); chromatographic column: SunFire@ Prep C18 (19 mm×250 mm; The sample was dissolved in DMF and filtered with a 0.45 m filter to prepare a sample solution. Preparative chromatography conditions: a. mobile phases A and B: mobile phase A: acetonitrile; mobile phase B: water (containing 5 mM ammonium acetate); b. gradient elution: the content of mobile phase A rises from 40% to 70%; c. flow rate: 15 mL/min; d. elution time: 20 min; retention time: 12.48 min.

LC-MS (ESI): m/z=618.6 [M+H]⁺.

1H NMR (400 MHz, Chloroform-d) δ 8.73 (d, 1H), 8.43 (d, 1H), 8.15 (dd, 1H), 8.03 (s, 1H), 7.70 (dd, 2H), 7.64-7.52 (m, 2H), 7.46 (dd, 2H), 5.72 (s, 2H), 5.08-4.57 (m, 2H), 4.09 (d, 4H), 3.29 (s, 2H), 3.03 (s, 2H), 2.04-1.71 (m, 2H), 1.28 (d, 1H).

### Example 96

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)propionamide

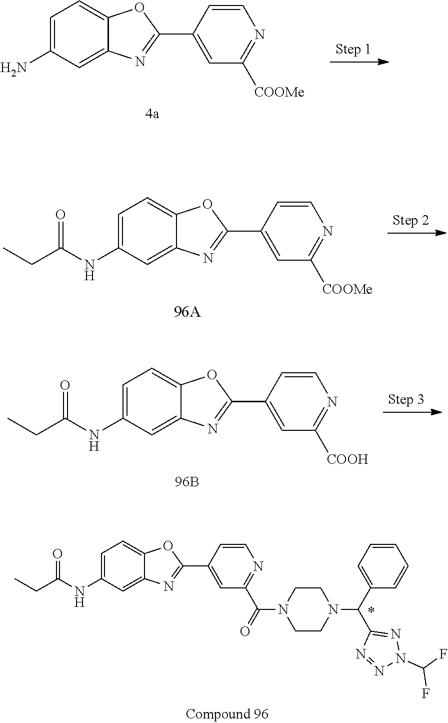 [SMILES: COC(=O)c1cc(-c2nc3cc(N)ccc3o2)ccn1 ; CCC(=O)Nc1ccc2oc(-c3ccnc(C(=O)OC)c3)nc2c1 ; CCC(=O)Nc1ccc2oc(-c3ccnc(C(=O)O)c3)nc2c1 ; CCC(=O)Nc1ccc2oc(-c3ccnc(C(=O)N4CCN(C(c5ccccc5)c5nnn(C(F)F)n5)CC4)c3)nc2c1]

Step 1:

### Methyl 4-(5-propionamidobenzo[d]oxazol-2-yl)picolinate (96A)

Compound 4a (500 mg, 1.86 mmol) was dissolved in 50 mL of dichloromethane; triethylamine (563 mg, 5.58 mmol) was added; and propionyl chloride (258 mg, 2.79 mmol) was added under an ice bath. The mixture was stirred at room temperature and reacted for 2 hours. After TLC showed that the reaction was completed, the reaction was quenched by adding water. The reaction solution was extracted with dichloromethane. The organic phase was dried and concentrated to obtain a crude product of 96A.

LC-MS (ESI): m/z=326.1 [M+H]⁺.

Step 2:

### 4-(5-propionamidobenzo[d]oxazol-2-yl)picolinic Acid (96B)

The crude product of 96A obtained in step 1 was dissolved in 50 mL of methanol, and an aqueous solution of lithium hydroxide (223 mg, 9.3 mmol) was added. The reaction was stirred at room temperature for 1 hour. After TLC showed that the reaction was completed, the reaction solution was concentrated, diluted by adding water and adjusted to pH 3 with 2 N dilute hydrochloric acid solution. The solid was precipitated in the reaction solution and filtered by suction, and the filter cake was dried to obtain 96B (425 mg, 73%).

LC-MS (ESI): m/z=311.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)propionamide (Compound 96)

96B (65 mg, 0.21 mmol) was dissolved in 5 mL of N,N-dimethylformamide; HATU (119 mg, 0.31 mmol), N,N-diisopropylethylamine (0.1 mL, 0.64 mmol) and intermediate 7 (121 mg, 0.31 mmol) were added; and the mixture was reacted at room temperature for 2 hours. After TLC showed that the reaction was completed, the reaction solution was extracted with ethyl acetate and water and washed twice with saturated brine. The organic phase was dried and concentrated to obtain a crude product of compound 96, which was subjected to preparative chromatography to obtain compound 96 (56 mg, 45%). Preparation method: instrument: waters 2767 (preparative liquid phase chromatographic instrument); chromatographic column: SunFire@ Prep C18 (19 mm×250 mm); The sample was dissolved in DMF and filtered with a 0.45 μm filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; c. flow rate: 15 ml/min; d elution time: 18 min; retention time: 10.56 min.

LC-MS (ESI): m/z=588.2 [M+H]⁺.

¹H NMR (400 MHz, CD₃Cl) δ 8.78 (d, 1H), 8.30 (s, 1H), 8.16-8.20 (m, 3H), 7.66-7.68 (m, 1H), 7.52-7.59 (m, 3H), 7.29-7.39 (m, 3H), 5.19 (s, 1H), 3.85 (s, 2H), 3.58 (s, 2H), 2.57-2.59 (m, 3H), 2.41-2.46 (m, 3H), 1.21-1.25 (m, 3H).

### Example 97

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)isobutyramide

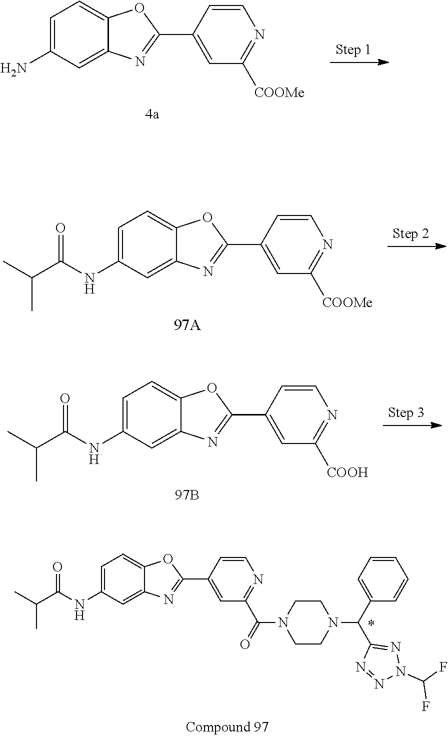 [SMILES: COC(=O)c1cc(-c2nc3cc(N)ccc3o2)ccn1 ; COC(=O)c1cc(-c2nc3cc(NC(=O)C(C)C)ccc3o2)ccn1 ; CC(C)C(=O)Nc1ccc2oc(-c3ccnc(C(=O)O)c3)nc2c1 ; CC(C)C(=O)Nc1ccc2oc(-c3ccnc(C(=O)N4CCN(C(c5ccccc5)c5nnn(C(F)F)n5)CC4)c3)nc2c1]

Step 1:

### Methyl 4-(5-propionamidobenzo[d]oxazol-2-yl)picolinate (97A)

Compound 4a (500 mg, 1.86 mmol) was dissolved in 50 mL of dichloromethane; triethylamine (563 mg, 5.58 mmol) was added; and isobutyryl chloride (297 mg, 2.79 mmol) was added under an ice bath. The mixture was stirred at room temperature and reacted for 2 hours. After TLC showed that the reaction was completed, the reaction was quenched by adding water. The reaction solution was extracted with dichloromethane. The organic phase was dried and concentrated to obtain a crude product of 97A.

LC-MS (ESI): m/z=340.1 [M+H]⁺.

Step 2:

### 4-(5-propionamidobenzo[d]oxazol-2-yl)picolinic Acid (97B)

The crude product of 97A obtained in step 1 was dissolved in 50 mL of methanol, and an aqueous solution of lithium hydroxide (223 mg, 9.3 mmol) was added. The reaction was stirred at room temperature for 1 hour. After TLC showed that the reaction was completed, the reaction solution was concentrated, diluted by adding water and adjusted to pH 3 with 2 N dilute hydrochloric acid solution. The solid was precipitated in the reaction solution and filtered by suction, and the filter cake was dried to obtain 97B (525 mg, 87%).

LC-MS (ESI): m/z=326.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)isobutyramide (Compound 97)

97B (65 mg, 0.2 mmol) was dissolved in 5 mL of N,N-dimethylformamide. HATU (114 mg, 0.3 mmol), N,N-diisopropylethylamine (0.1 mL, 0.6 mmol) and intermediate 7 (119 mg, 0.3 mmol) were added, and the mixture was reacted at room temperature for 2 hours. After TLC showed that the reaction was completed, the reaction solution was extracted with ethyl acetate and water and washed twice with saturated brine. The organic phase was dried and concentrated to obtain a crude product of compound 97, which was subjected to preparative chromatography to obtain compound 97 (55 mg, 46%). Preparation method: instrument: waters 2767 (preparative liquid phase chromatographic instrument); chromatographic column: SunFire@ Prep C18 (19 mm×250 mm); The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.11 min.

LC-MS (ESI): m/z=602.2 [M+H]⁺.

¹H NMR (400 MHz, CD₃Cl) δ 8.78 (d, 1H), 8.30 (s, 1H), 8.16-8.19 (m, 3H), 7.66-7.68 (m, 1H), 7.52-7.60 (m, 3H), 7.24-7.39 (m, 3H), 5.19 (s, 1H), 3.85 (s, 2H), 3.58 (s, 2H), 2.41-2.70 (m, 5H), 1.22-1.24 (m, 6H).

### Example 98

### N-(2-(2-(4-((2-methyl-2H-tetrazol-5-yl)(thiazol-2-yl)methyl)piperazine-1-carbonyl)pyridin-4-yl)benzo[d]oxazol-5-yl)acetamide

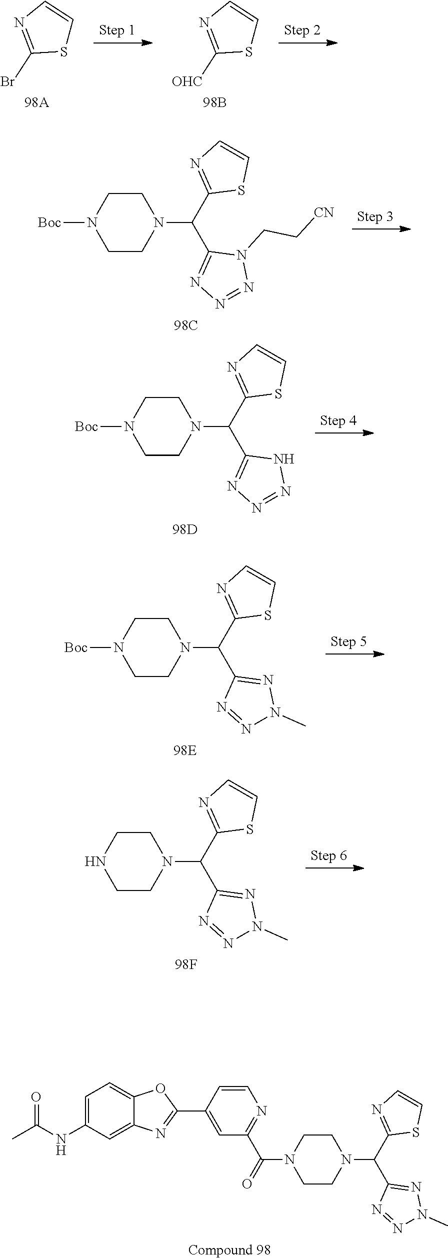

Step 1:

### thiazole-2-carbaldehyde (98B)

2-bromothiazole (3 g, 18.29 mmol) was added to a 250 ml three-necked flask and dissolved in diethyl ether (12 mL). Under nitrogen protection, n-BuLi (10 ml, 25.00 mmol) was added dropwise at −70° C., and the mixture was reacted at −70° C. for 0.5 h. DMF (2.01 g, 27.44 mmol) was added, and the mixture was reacted at −55° C. for 2 h. 4 mol/L hydrochloric acid was added under an ice bath, and liquid separation was performed. The organic phase was washed by adding 4 mol/L of hydrochloric acid. The aqueous phase was collected and adjusted to pH=10 by adding saturated K₂CO₃. The resulting solution was extracted three times with diethyl ether. The organic phase was collected, washed with saturated brine, dried over anhydrous sodium sulfate and concentrated to obtain 98B (1.07 g, 52%).

LC-MS (ESI): m/z=114.1 [M+H]⁺.

Step 2:

### tert-butyl-4-((1-(2-cyanoethyl)-1H-tetrazol-5-yl)(thiazol-2-yl)methyl)piperazine-1-carboxylate (98C)

To a 100 ml round bottom flask, thiazole-2-carbaldehyde (3 g, 26.52 mmol) and tert-butyl piperazine-1-carboxylate (4.94 g, 26.52 mmol) were added and dissolved in MeOH (15 mL). The mixture was reacted at room temperature for 20 min. 3-isocyanopropanenitrile (2.12 g, 26.52 mmol) and TMSN₃ (azidotrimethylsilane) (3.05 g, 26.52 mmol) were added. The mixture was reacted at room temperature overnight. After LCMS monitored a great amount of products, the reaction solution was used directly in the next step.

LC-MS (ESI): m/z=405.1 [M+H]⁺.

Step 3:

### tert-butyl 4-((1H-tetrazol-5-yl)(thiazol-2-yl)methyl)piperazine-1-carboxylate (98D)

To a 100 ml round bottom flask, the reaction solution from the previous step, LiOH (0.71 g, 29.67 mmol) and water (20 mL) were added, and the mixture was reacted at room temperature for 3 h. The reaction solution was extracted with DCM. The aqueous phase was collected and adjusted to pH=3 or so by adding dilute hydrochloric acid. The aqueous phase was extracted with DCM, and the organic phase was collected, washed with saturated brine, dried over anhydrous sodium sulfate and concentrated to obtain 98D (3 g, 35%).

LC-MS (ESI): m/z=352.2 [M+H]⁺.

Step 4:

### tert-butyl-4-((2-methyl-2H-tetrazol-5-yl)(thiazol-2-yl)methyl)piperazine-1-carboxylate (98E)

To a 100 ml round bottom flask, tert-butyl 4-((1H-tetrazol-5-yl)(thiazol-2-yl)methyl)piperazine-1-carboxylate (3 g, 8.54 mmol) was added and dissolved in a mixed solvent of THF/MeOH (4:1) (10 mL). TMSCHN₂ (trimethylsilazodimethane) (6.5 mL, 13 mmol) was added, and the mixture was reacted at room temperature for 2 h. The reaction solution was concentrated by column chromatography (eluent: PE:EA=1:1) to obtain 98E (0.66 g, 21%).

LC-MS (ESI): m/z=366.2 [M+H]⁺.

Step 5:

### 2-((2-methyl-2H-tetrazol-5-yl)(piperazin-1-yl)methyl)thiazole (98F)

To a 100 ml round bottom flask, tert-butyl 4-((2-methyl-2H-tetrazol-5-yl)(thiazole-2-yl)methyl)piperazine-1-carboxylate (0.66 g, 1.80 mmol) was added and dissolved in a mixed solvent of TFA/DCM (2:1) (6 mL), and the mixture was reacted at room temperature for 3 h. The reaction solution was concentrated to obtain a crude product of 98F (0.8 g).

LC-MS (ESI): m/z=266.2 [M+H]⁺.

Step 6:

### N-(2-(2-(4-((2-methyl-2H-tetrazol-5-yl)(thiazol-2-yl)methyl)piperazine-1-carbonyl)pyridin-4-yl)benzo[d]oxazol-5-yl)acetamide (Compound 98)

To a 100 ml round bottom flask, 2-((2-methyl-2H-tetrazol-5-yl)(piperazin-1-yl)methyl)thiazole (0.4 g, 1.51 mmol), 4-(5-acetamido-1,3-benzoxazol-2-yl)pyridine-2-carboxylic acid (0.45 g, 1.51 mmol), HATU (0.86 g, 2.26 mmol) and DIPEA (0.58 g, 4.52 mmol) were added and dissolved in DMF (8 mL), and the mixture was reacted at room temperature overnight. The reaction was diluted by adding water and extracted with ethyl acetate, and the organic phase was collected, washed with saturated brine, dried over anhydrous sodium sulfate and subjected to prep-HPLC to obtain compound 98 (147 mg, 18%). Preparation conditions: instrument: waters 2767 (preparative liquid phase chromatographic instrument); chromatographic column: XBridge C18 5 μm, 19×250 mm. The sample was dissolved in acetonitrile and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 5% to 50%; time: 15 min; flow rate: 12 ml/min. Components with retention time of 9.8 min were used

LC-MS (ESI): m/z=545.2 [M+H]⁺.

¹H NMR (400 MHz, DMSO-d6) δ 10.16 (s, 1H), 8.81 (d, 1H), 8.22-8.13 (m, 3H), 7.79-7.72 (m, 3H), 7.59-7.57 (m, 1H), 5.74 (s, 1H), 4.39 (s, 3H), 3.76-3.72 (m, 2H), 3.44-3.49 (m, 2H), 2.71-2.54 (m, 4H), 2.09 (s, 3H)

### Example 99

### N-(2-(2-(4-(1-(2-(difluoromethyl)-2H-tetrazol-5-yl)-2,2-difluoro-1-(thiazol-2-yl)ethyl)piperazine-1-carbonyl)pyridin-4-yl)benzo[d]oxazol-5-yl)acetamide

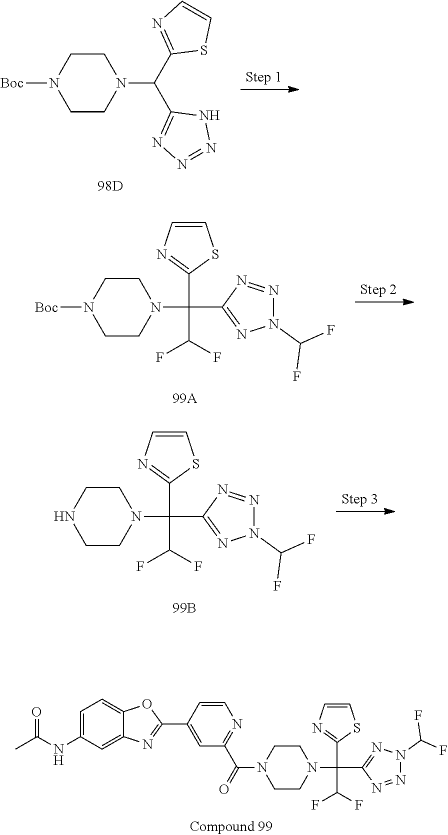 [SMILES: CC(C)(C)OC(=O)N1CCN(C(c2nnn[nH]2)c2nccs2)CC1 ; CC(C)(C)OC(=O)N1CCN(C(c2nnn(C(F)F)n2)(c2nccs2)C(F)F)CC1 ; FC(F)n1nnc(C(c2nccs2)(C(F)F)N2CCNCC2)n1 ; CC(=O)Nc1ccc2oc(-c3ccnc(C(=O)N4CCN(C(c5nnn(C(F)F)n5)(c5nccs5)C(F)F)CC4)c3)nc2c1]

Step 1:

### tert-butyl4-(1-(2-(difluoromethyl)-2H-tetrazol-5-yl)-2,2-difluoro-1-(thiazol-2-yl)ethyl)piperazine-1-carboxylate (99A)

To a 100 ml round bottom flask, tert-butyl 4-((1H-tetrazol-5-yl)(thiazol-2-yl)methyl)piperazine-1-carboxylate (0.5 g, 1.42 mmol), diethyl(bromodifluoromethyl)phosphonate (0.49 g, 1.85 mmol) and KOH (1.60 g, 28.46 mmol) were added and dissolved in a mixed solvent of ACN/H₂O (1:1) (6 mL), and the mixture was reacted at room temperature overnight. The reaction solution was diluted by adding dichloromethane and water, and liquid separation was performed. The aqueous phase was extracted once by adding dichloromethane; the organic phase was combined, washed twice with saturated brine and concentrated; and the residue was purified on a preparative plate (developing solvent PE:EA=1:1) to obtain 99A (0.17 g, 26%).

LC-MS (ESI): m/z=452.1 [M+H]⁺.

Step 2:

### 2-(1-(2-(difluoromethyl)-2H-tetrazol-5-yl)-2,2-difluoro-1-(piperazin-1-yl)ethyl)thiazole (99B)

Intermediate 99A was used as a raw material to obtain compound 99B (130 mg) with reference to the synthetic step (step 5) of example 98.

LC-MS (ESI): m/z=352.1 [M+H]⁺.

Step 3:

### N-(2-(2-(4-(1-(2-(difluoromethyl)-2H-tetrazol-5-yl)-2,2-difluoro-1-(thiazol-2-yl)ethyl)piperazine-1-carbonyl)pyridin-4-yl)benzo[d]oxazol-5-yl)acetamide (Compound 99)

Intermediate 99B was used as a raw material to obtain compound 99 (56 mg, 21%) with reference to the synthetic step (step 6) of example 98.

LC-MS (ESI): m/z=631.1 [M+H]⁺.

1H NMR (400 MHz, DMSO-d₆) δ 10.16 (s, 1H), 9.00-8.72 (m, 2H), 8.22-8.07 (m, 4H), 7.79-7.76 (m, 1H), 7.70-7.44 (m, 3H), 3.80-3.76 (m, 2H), 3.62-3.55 (m, 2H), 2.82-2.61 (m, 4H), 2.09 (s, 3H).

### Example 100

### (R/S)-1-(2-(2-(4-((2-(difluoromethyl)-2H-tetrazol-5-yl)(phenyl)methyl)piperazine-1-carbonyl)pyridin-4-yl)-6,7-dihydrooxazolo[4,5-c]pyridin-5(4H)-yl)ethan-1-one

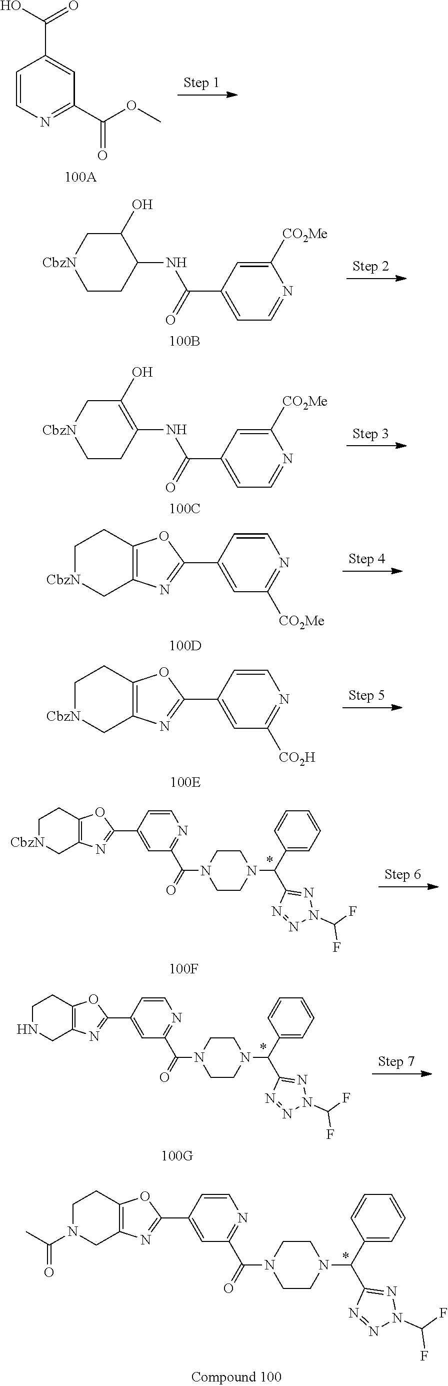

Step 1:

### Methyl 4-((1-((benzyloxy)carbonyl)-3-hydroxypiperidin-4-yl)carbamoyl)picolinate (100B)

100A (1.20 g, 6.6 mmol) and benzyl 4-amino-3-hydroxypiperidine-1-carboxylate (1.65 g, 6.6 mmol) were dissolved in dichloromethane (20 mL) at room temperature, and HATU (3.00 g, 8.0 mmol) was added. The mixture was mixed evenly, and then DIEA (1.50 g, 12.4 mmol) was added dropwise. After completion of the dropwise addition, the reaction was stirred at room temperature for 16 hours. The reaction solution was diluted with dichloromethane (30 ml), washed with 0.5 mol/L of hydrochloric acid aqueous solution (20 ml) and saturated brine. The organic phase was separated, dried over anhydrous sodium sulfate, concentrated under reduced pressure and subjected to column chromatography (petroleum ether:ethyl acetate=3:1) to obtain 100B (200 mg, 55.2%).

LC-MS (ESI): m/z=414.2[M+H]⁺.

Step 2:

### Methyl 4-((1-((benzyloxy)carbonyl)-5-hydroxy-1,2,3,6-tetrahydropyridin-4-yl)carbamoyl)picolinate (100C)

100B (1.50 g, 3.6 mmol) was added to dichloromethane (20 ml) at room temperature, and Dess-Martin reagent (1.70 g) was added in portions. After the addition was completed, the reaction was stirred at room temperature for 4 hours; the reaction solution was diluted by adding dichloromethane (20 ml), washed with saturated sodium bicarbonate (20 ml) and saturated brine (20 ml), dried over anhydrous sodium sulfate and concentrated under reduced pressure; and the residue was purified by column chromatography (petroleum ether:ethyl acetate=2:1) to obtain 100C (1.3 g, 86.7%).

LC-MS (ESI): m/z=412.2[M+H]⁺.

Step 3:

### Benzyl 2-(2-(methoxycarbonyl)pyridin-4-yl)-6,7-dihydrooxazolo[4,5-c]pyridine-5(4H)-carboxylate (100D)

100C (1.10 g, 2.67 mmol) was dissolved in 1,4-dioxane (20 mL) at room temperature, and then phosphorus oxychloride (0.5 mL, 30.0 mmol) was added dropwise with stirring. The reaction was warmed to 90° C. and stirred; after 6 hours, the heating was stopped, and the mixture was naturally cooled to room temperature. The reaction solution was concentrated, and ethyl acetate (30 ml) was added to the residue. The mixture was washed with saturated sodium bicarbonate (20 ml) and saturated brine (20 ml). The reaction solution was subjected to rotary evaporation and then column chromatography (PE:EA=1:1) to obtain 100D (200 mg, 19.1%).

LC-MS (ESI): m/z=394.2 [M+H]⁺.

Step 4:

### 4-(5-((benzyloxy)carbonyl)-4,5,6,7-tetrahydrooxazolo[4,5-c]pyridin-2-yl)picolinic Acid (100E)

At room temperature, 100D (200 mg, 0.5 mmol) was added to a mixed solvent (4 ml) of methanol:tetrahydrofuran:water=2:1:1, and lithium hydroxide monohydrate (42 mg, 1.0 mmol) was added. The reaction was stirred at room temperature for 16 hours, and then the reaction solution was concentrated. The residue was added to water (10 ml), and the impurities were extracted once with ethyl acetate (10 ml). The aqueous phase was adjusted to pH=3 or so with dilute hydrochloric acid and extracted with ethyl acetate (10 ml×2). The organic phase was combined, washed with saturated brine (10 ml), dried over anhydrous sodium sulfate and concentrated to obtain 100E (160 mg, 84.2%).

LC-MS (ESI): m/z=380.2 [M+H]⁺.

Step 5:

### Benzyl (R/S)-2-(2-(4-((2-(difluoromethyl)-2H-tetrazol-5-yl)(phenyl)methyl)piperazine-1-carbonyl)pyridin-4-yl)-6,7-dihydrooxazolo[4,5-c]pyridine-5(4H)-carboxylate (100F)

100E (130 mg, 0.34 mmol) and intermediate 7 (100 mg, 0.34 mmol) were added to dichloromethane (5 ml) at room temperature and mixed evenly; HATU (150 mg, 0.40 mmol) was added; and then DIEA (0.1 ml, 0.8 mmol) was added dropwise. The reaction was stirred at room temperature for 16 hours, and then the reaction solution was diluted with dichloromethane (20 ml) and washed successively with water (10 ml), dilute hydrochloric acid (0.5 N, 10 ml) and saturated brine. The organic phase was dried over anhydrous sodium sulfate and concentrated, and then the residue was purified by column chromatography (ethyl acetate:petroleum ether=1:1) to obtain 100F (120 mg, 53.8%).

LC-MS (ESI): m/z=656.2 [M+H]⁺.

Step 6:

### (R/S)-(4-((2-(difluoromethyl)-2H-tetrazol-5-yl)(phenyl)methyl)piperazin-1-yl)(4-(4,5,6,7-tetrahydrooxazolo[4,5-c]pyridin-2-yl)pyridin-2-yl)methanone (100G)

100F (100 mg, 0.15 mmol) was added to methanol (2 ml), and a catalytic amount of 10% palladium on carbon catalyst was added. The mixture was subjected to hydrogen replacement 3 times, and then the reaction was stirred under hydrogen atmosphere for 16 hours. The reaction solution was filtered through a small amount of celite. The filtrate was concentrated to obtain 100G (70 mg, 87.5%).

LC-MS (ESI): m/z=522.2 [M+H]⁺.

Step 7:

### (R/S)-1-(2-(2-(4-((2-(difluoromethyl)-2H-tetrazol-5-yl)(phenyl)methyl)piperazine-1-carbonyl)pyridin-4-yl)-6,7-dihydrooxazolo[4,5-c]pyridin-5(4H)-yl)ethan-1-one (Compound 100)

100G (50 mg, 0.075 mmol) was added to dichloromethane (2 ml); triethylamine (15 mg, 0.15 mmol) was added; and acetyl chloride (10 mg, 0.125 mmol) was added dropwise. The mixture was stirred at room temperature for 2 hours, diluted by adding dichloromethane, washed with saturated brine, dried over anhydrous sodium sulfate and concentrated, and the residue was separated by a preparative plate to obtain compound 100 (20 mg, 37.5%).

LC-MS (ESI): m/z=564.2 [M+H]⁺.

¹H NMR (400 MHz, CDCl₃) δ8.63 (t, 1H), 8.15 (s, 1H), 7.85 (t, 1H), 7.61 (t, 1H), 7.52 (d, 2H), 7.38-7.31 (m, 3H), 5.1 (s, 1H), 4.66 (s, 1H), 4.51 (s, 1H), 4.01-3.98 (m, 1H), 3.88-3.80 (m, 3H), 3.63-3.65 (m, 2H), 2.95-2.85 (m, 2H), 2.72-2.38 (m, 4H), 2.20 (s, 3H).

### Example 101

### (R/S)-(4-((2-(difluoromethyl)-2H-tetrazol-5-yl)(phenyl)methyl)piperazin-1-yl)(4-(5-(isothiazol-4-yl)benzo[d]oxazol-2-yl)pyridin-2-yl)methanone

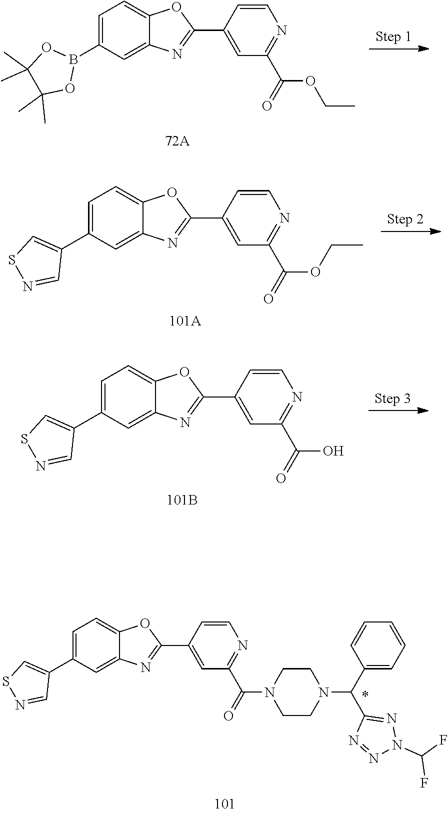

Step 1:

### Ethyl 4-(5-(isothiazol-4-yl)benzo[d]oxazol-2-yl)picolinate (101A)

Under nitrogen protection, 72A (100.0 mg, 0.25 mmol) was dissolved in DMF (5.0 ml), and 4-bromoisoxazole (37.0 mg, 0.25 mmol) and Pd(dppf)Cl₂ (36 mg, 0.05 mmol) were added. After the addition, the mixture was reacted at 100° C. under microwave for 1 hour and cooled, and then the reaction solution was poured into ice water (20 ml) and extracted with ethyl acetate (50 ml×2). The organic layer was combined, washed with water and brine, dried and concentrated, and the residue was purified by column purification (eluent: EA/PE=10%-30%) to obtain compound 101A (40 mg, 43%).

LC-MS (ESI): m/z=352.1 [M+H]⁺.

Step 2:

### 4-(5-(isothiazol-4-yl)benzo[d]oxazol-2-yl)picolinic Acid (101B)

Compound 101A (35 mg, 0.1 mmol) was dissolved in methanol (2 ml), and sodium hydroxide (8 mg, 0.2 mmol, 0.5 ml) aqueous solution was added. The mixture was stirred at room temperature for 3 hours, concentrated under reduced pressure to remove most of methanol, adjusted to pH=2-3 by dropwise adding 1 N hydrochloric acid and filtered. The filter cake was washed with water (3 mL) and dried to obtain compound 101B (25 mg, 72%).

LC-MS (ESI): m/z=324.1[M+H]⁺.

Step 3:

### (R/S)-(4-((2-(difluoromethyl)-2H-tetrazol-5-yl)(phenyl)methyl)piperazin-1-yl)(4-(5-(isothiazol-4-yl)benzo[d]oxazol-2-yl)pyridin-2-yl)methanone (Compound 101)

Compound 101B (25.0 mg, 0.08 mmol) was dissolved in N,N-dimethylformamide (2 ml), and the mixture was cooled to 0° C.; HATU (38.0 mg, 0.1 mmol) and DIPEA (51.0 mg, 0.4 mmol) were added; and then intermediate 7 (30.0 mg, 0.1 mmol) was added. After the addition, the mixture was stirred for 1 hour while the temperature was controlled at 0° C., and the reaction was quenched by adding ice water (5 ml). The resulting solution was extracted twice with dichloromethane (20 ml×2). The organic phase was combined, dried over anhydrous sodium sulfate and concentrated under reduced pressure, and then the residue was separated by column chromatography (eluent: MeOH/DCM=5%-10%) to obtain compound 101 (18 mg, 38%).

LC-MS (ESI): m/z=600.2[M+H]⁺.

¹H NMR (400 MHz, CDCl₃) δ 8.82 (s, 1H), 8.77 (s, 1H), 8.75 (d, 1H), 8.44 (s, 1H), 8.12 (d, 1H), 8.02 (s, 1H), 7.77-7.48 (m, 5H), 7.39-7.33 (m, 3H), 5.17 (s, 1H), 3.93-3.74 (m, 4H), 2.79-2.48 (m, 4H).

### Example 102

### (4-((R/S)-(2-(difluoromethyl)-2H-tetrazol-5-yl)(phenyl)methyl)piperazin-1-yl)(4-(6-(((S)-tetrahydrofuran-3-yl)oxy)benzo[d]oxazol-2-yl)pyridin-2-yl)methanone

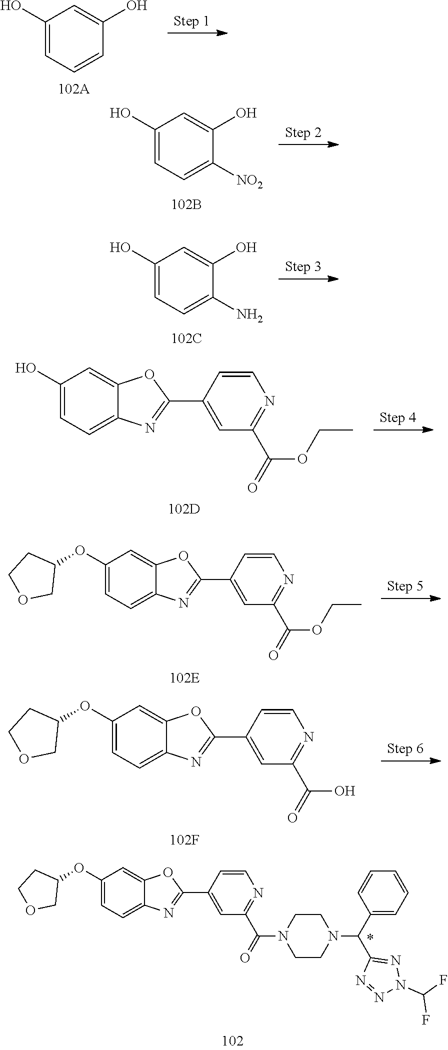

Step 1:

### 4-nitrobenzene-1,3-diol (102B)

Compound 102A (5.0 g, 45.5 mmol) was dissolved in a mixed solvent of dichloromethane (200 ml) and acetic acid (90 ml). The mixture was cooled to 0-5° C., and nitric acid (4.43 g, 47.8 mmol, a content of 68%) was slowly added dropwise. After the dropwise addition was completed, the resulting mixture was reacted at room temperature for 1 hour, and the reaction solution was poured into ice water (500 ml) and extracted twice with dichloromethane (200 ml×2). The organic layer was combined, washed with water and saturated brine, dried over anhydrous sodium sulfate and concentrated under reduced pressure, and the residue was separated by column chromatography (eluent: EA/PE=10%-30%) to obtain compound 102B (3.2 g, 45%).

LC-MS (ESI): m/z=156.0[M+H]⁺.

¹H NMR (400 MHz, DMSO-d6) δ 10.93 (s, 1H), 10.79 (s, 1H), 7.90 (d, 1H), 6.45-6.40 (m, 2H).

Step 2:

### 4-aminobenzene-1,3-diol (102C)

Compound 102B (3.2 g, 20.3 mmol) was dissolved in methanol (50 ml); Pd/C (0.5 g, a content of 10%) was added; and hydrogen was introduced. The mixture was stirred at room temperature for 2 hours and filtered under nitrogen protection. The filtrate was concentrated to obtain compound 102C (2.4 g, 94%).

LC-MS (ESI): m/z=126.0[M+H]⁺.

Step 3:

### Ethyl 4-(6-hydroxy-2,3-dihydrobenzo[d]oxazol-2-yl)picolinate (102D)

Compound 102C (1.0 g, 8.0 mmol) was dissolved in anhydrous ethanol (20 ml), and ethyl 4-formylpicolinate (1.4 g, 8 mmol) was added. The mixture was warmed to 70° C., reacted for 5 hours and concentrated under reduced pressure to remove ethanol. Dichloromethane (50 ml) and DDQ (2.2 g, 9.6 mmol) were added, and the resulting mixture was stirred at room temperature for 2 hours. After completion of the reaction, the reaction solution was poured into ice water (100 ml), extracted with dichloromethane (100 ml×2), washed with saturated aqueous sodium bicarbonate solution and brine, dried over anhydrous sodium sulfate, filtered and concentrated. The residue was purified by column purification (eluent: EA/PE=20%-40%) to obtain compound 102D (0.8 g, 35%).

LC-MS (ESI): m/z=285.1[M+H]⁺.

Step 4:

### Ethyl (S)-4-(6-((tetrahydrofuran-3-yl)oxy)benzo[d]oxazol-2-yl)picolinate (102E)

Compound 102D (0.8 g, 2.8 mmol) was dissolved in DMF (10 ml), and cesium carbonate (1.8 g, 5.6 mmol) and (R)-tetrahydrofuran-3-yl-methanesulfonate (564 mg, 3.4 mmol) was added. After the addition, the mixture was warmed to 90° C. and reacted for 4 hours. After completion of the reaction, the reaction solution was poured into ice water (50 ml), extracted with ethyl acetate (100 ml×2), washed with brine, dried over anhydrous sodium sulfate, filtered and concentrated. The residue was purified by column purification (eluent: EA/PE=10%-30%) to obtain compound 102E (0.7 g, 71%).

LC-MS (ESI): m/z=355.1[M+H]⁺.

Step 5:

### (S)-4-(6-((tetrahydrofuran-3-yl)oxy)benzo[d]oxazol-2-yl)picolinic Acid (102F)

Compound 102E (200.0 mg, 0.56 mmol) was dissolved in methanol (10 ml), and hydroxide (45 mg, 1.12 mmol, 2.0 ml) aqueous solution was added. The mixture was stirred at room temperature for 3 hours, concentrated under reduced pressure to remove most of methanol, adjusted to pH=2-3 by dropwise adding 1 N hydrochloric acid and filtered. The filter cake was washed with water (3 mL) and dried to obtain compound 102F (160 mg, 87%).

LC-MS (ESI): m/z=327[M+H]⁺.

Step 6:

### (4-((R/S)-(2-(difluoromethyl)-2H-tetrazol-5-yl)(phenyl)methyl)piperazin-1-yl)(4-(6-(((S)-tetrahydrofuran-3-yl)oxy)benzo[d]oxazol-2-yl)pyridin-2-yl)methanone (Compound 102)

Compound 102F (160.0 mg, 0.49 mmol) was dissolved in N,N-dimethylformamide (5 ml), and the mixture was cooled to 0° C.; HATU (223.0 mg, 0.59 mmol) and DIPEA (253.0 mg, 1.96 mmol) were added; and then intermediate 7 (145.0 mg, 0.49 mmol) was added. After the addition, the mixture was stirred for 1 hour while the temperature was controlled at 0° C., and then the reaction was quenched by adding ice water (30 ml). The resulting solution was extracted twice with dichloromethane (50 ml×2). The organic phase was combined, dried over anhydrous sodium sulfate and concentrated under reduced pressure, and then the residue was separated by column chromatography (eluent: EA/DCM=30%-60%) to obtain compound 102 (98 mg, 33%).

LC-MS (ESI): m/z=603.2[M+H]⁺.

¹H NMR (400 MHz, CDCl₃) δ 8.70 (d, 1H), 8.37 (s, 1H), 8.07-8.05 (m, 1H), 7.77-7.38 (m, 4H), 7.41-7.35 (m, 3H), 7.08 (d, 1H), 7.00-6.97 (m, 1H), 5.27 (s, 1H), 5.01-4.98 (m, 1H), 3.96-3.80 (m, 8H), 2.86-2.56 (m, 4H), 2.77-2.17 (m, 2H).

### Example 103

### (S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-6-yl)-2,2-difluorocyclopropane-1-carboxamide

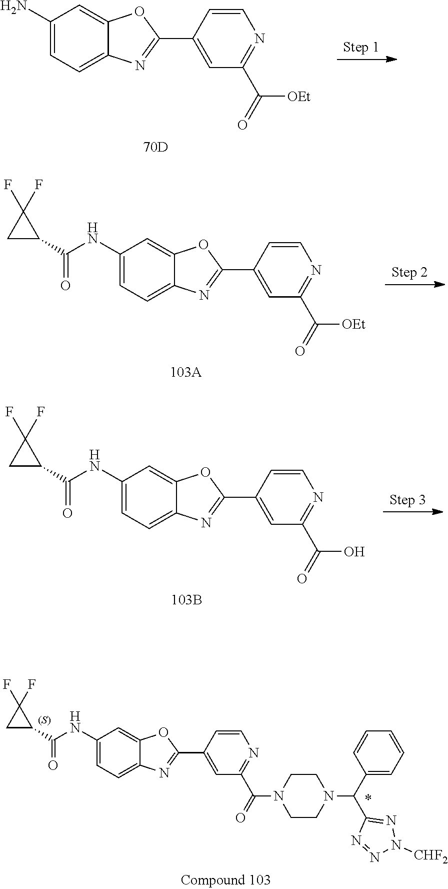

Step 1:

### Ethyl (S)-4-(6-(2,2-difluorocyclopropane-1-carboxamido)benzo[d]oxazol-2-yl)picolinate (103A)

At room temperature, DMF (15 mL), (S)-2,2-difluorocyclopropane-1-carboxylic acid (172 mg, 1.41 mmol), HATU (804 mg, 2.12 mmol) and DIPEA (546 mg, 4.23 mmol) were successively added to compound 70D (400 mg, 1.41 mmol). The reaction was stirred for 1 hour. The reaction solution was poured into water (50 mL) and extracted with ethyl acetate (20 mL×3). The organic phase was concentrated, and the residue was separated and purified by silica gel column chromatography (PE:EA=1:5) to obtain compound 103A (450 mg, 82%).

LC-MS (ESI): m/z=388.1 [M+H]⁺.

Step 2:

### (S)-4-(6-(2,2-difluorocyclopropane-1-carboxamido)benzo[d]oxazol-2-yl)picolinic Acid (103B)

Anhydrous methanol (10 mL) and NaOH (103 mg, 2.58 mmol, 2 mL) aqueous solution were successively added to compound 103A (0.20 g, 0.52 mmol). The mixture was stirred at 25° C. for 10 h, concentrated under reduced pressure to remove most of methanol, adjusted to pH=2-3 by dropwise adding 1 N hydrochloric acid and filtered. The filter cake was washed with water (1 mL) and dried to obtain intermediate 103B (130 mg, 70%).

LC-MS (ESI): m/z=360.1 [M+H]⁺.

Step 3:

### (S)—N-(2-(2-(4-((R/S)-(2-(difluoromethyl)-2H-tetrazol-5-yl)(phenyl)methyl)piperazine-1-carbonyl)pyridin-4-yl)benzo[d]oxazol-6-yl)-2,2-difluorocyclopropane-1-carboxamide (Compound 103)

At room temperature, DMF (5 mL), intermediate 7 (106 mg, 0.36 mmol), HATU (205 mg, 0.54 mmol) and DIPEA (139 mg, 1.08 mmol) were successively added to compound 103B (130 mg, 0.36 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 103 (30 mg, 13%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.76 min.

LC-MS (ESI): m/z=636.3 [M+H]⁺.

¹H NMR (400 MHz, DMSO-d₆) δ 10.79 (s, 1H), 8.78 (d, 1H), 8.55 (t, 1H), 8.28 (s, 1H), 8.15-8.16 (m, 1H), 8.11-8.13 (m, 1H), 7.85 (d, 1H), 7.48-7.50 (m, 3H), 7.37-7.40 (m, 2H), 7.33-7.39 (m, 1H), 5.32 (s, 1H), 3.70-3.71 (m, 2H), 3.48-3.49 (m, 2H), 2.82-2.88 (m, 1H), 2.58-2.67 (m, 2H), 2.36-2.37 (m, 2H), 2.03-2.08 (m, 2H).

### Example 104

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-6-yl)cyclopropanecarboxamide

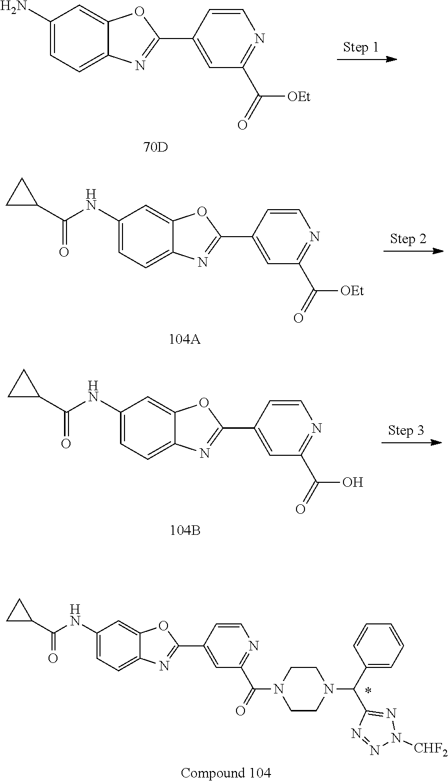 [SMILES: CCOC(=O)c1cc(-c2nc3ccc(N)cc3o2)ccn1 ; CCOC(=O)c1cc(-c2nc3ccc(NC(=O)C4CC4)cc3o2)ccn1 ; O=C(O)c1cc(-c2nc3ccc(NC(=O)C4CC4)cc3o2)ccn1 ; O=C(Nc1ccc2nc(-c3ccnc(C(=O)N4CCN(C(c5ccccc5)c5nnn(C(F)F)n5)CC4)c3)oc2c1)C1CC1]

Step 1:

### Ethyl 4-(6-(cyclopropanecarboxamido)benzo[d]oxazol-2-yl)picolinate (104A)

At room temperature, DCM (10 mL), cyclopropanecarbonyl chloride (147 mg, 1.41 mmol) and DIPEA (275 mg, 2.13 mmol) were successively added to compound 70D (200 mg, 0.71 mmol). The reaction was stirred for 1 hour. The reaction solution was poured into water (30 mL) and extracted with dichloromethane (20 mL×2). The organic phase was concentrated, and the residue was separated and purified by silica gel column chromatography (PE:EA=1:5) to obtain compound 104A (180 mg, 72%).

LC-MS (ESI): m/z=352.1 [M+H]⁺.

Step 2:

### 4-(6-(cyclopropanecarboxamido)benzo[d]oxazol-2-yl)picolinic Acid (104B)

Anhydrous methanol (10 mL) and NaOH (103 mg, 2.56 mmol, 2 mL) aqueous solution were successively added to compound 104A (0.18 g, 0.51 mmol). The mixture was stirred at 25° C. for 10 h, concentrated under reduced pressure to remove most of methanol, adjusted to pH=2-3 by dropwise adding 1 N hydrochloric acid and filtered. The filter cake was washed with water (1 mL) and dried to obtain intermediate 104B (100 mg, 61%).

LC-MS (ESI): m/z=324.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-6-yl)cyclopropanecarboxamide (Compound 104)

At room temperature, DMF (5 mL), intermediate 7 (91 mg, 0.31 mmol), HATU (177 mg, 0.47 mmol) and DIPEA (120 mg, 0.93 mmol) were successively added to compound 104B (100 mg, 0.31 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 104 (30 mg, 16%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.90 min.

LC-MS (ESI): m/z=600.3 [M+H]⁺.

¹H NMR (400 MHz, DMSO-d₆) δ 10.56 (s, 1H), 8.78 (d, 1H), 8.55 (t, 1H), 8.52-8.55 (m, 1H), 8.31-8.32 (m, 1H), 8.15-8.16 (m, 1H), 8.11-8.12 (m, 1H), 7.81 (d, 1H), 7.48-7.52 (m, 2H), 7.33-7.41 (m, 3H), 5.31 (s, 1H), 3.70-3.71 (m, 2H), 3.48-3.49 (m, 2H), 2.51-2.54 (m, 1H), 2.46-2.50 (m, 2H), 2.33-2.36 (m, 1H), 1.82-1.84 (m, 1H), 0.83-0.85 (m, 4H).

### Example 105

### (R/S)-(4-((2-(difluoromethyl)-2H-tetrazol-5-yl)(phenyl)methyl)piperazin-1-yl)(4-(5-(1-methyl-1H-pyrazol-4-yl)benzo[d]oxazol-2-yl)pyridin-2-yl)methanone

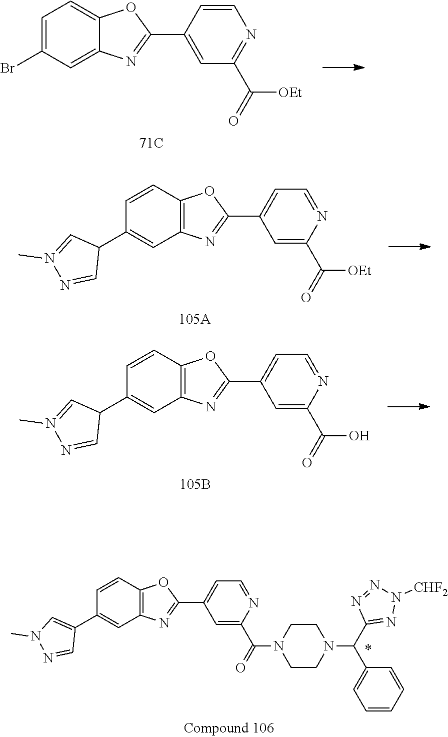

Step 1:

### Ethyl 4-(5-(1-methyl-1H-pyrazol-4-yl)benzo[d]oxazol-2-yl)picolinate (105A)

Compound 71C (250 mg, 0.75 mmol) was added to a mixed solvent of dioxane (10 mL) and water (1 mL), and then 1-methyl-4-(4,4,5,5-tetramethyl-1,3,2-dioxin-2-yl)-1H pyrazole (312 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:5) to obtain 105A (220 mg, 88%).

LC-MS (ESI): m/z=348.1[M+H]⁺.

Step 2:

### 4-(5-(1-methyl-1H-pyrazol-4-yl)benzo[d]oxazol-2-yl)picolinic Acid (105B)

Anhydrous methanol (10 mL) and NaOH (132 mg, 3.29 mmol, 2 mL) aqueous solution were successively added to compound 105A (220 mg, 0.66 mmol), and the mixture was stirred at 25° C. for 10 h. TLC showed that the reaction of the raw materials was completes. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 105B (200 mg, 95%).

LC-MS (ESI): m/z=321.1 [M+H]⁺.

Step 3:

### (R/S)-(4-((2-(difluoromethyl)-2H-tetrazol-5-yl)(phenyl)methyl)piperazin-1-yl)(4-(5-(1-methyl-1H-pyrazol-4-yl)benzo[d]oxazol-2-yl)pyridin-2-yl)methanone (Compound 105)

At room temperature, DMF (8 mL), intermediate 7 (110 mg, 0.38 mmol), HATU (217 mg, 0.57 mmol) and DIPEA (147 mg, 1.14 mmol) were successively added to compound 105B (120 mg, 0.38 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 105 (50 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 14.34 min.

LC-MS (ESI): m/z=597.3 [M+H]⁺.

¹H NMR (400 MHz, DMSO-d₆) δ8.81-8.82 (m, 1H), 8.55 (t, 1H), 8.23 (s, 1H), 8.18-8.19 (m, 1H), 8.14-8.15 (m, 1H), 8.07 (d, 1H), 7.97-7.98 (m, 1H), 7.83 (d, 1H), 7.71-7.74 (m, 1H), 7.49-7.51 (m, 2H), 7.37-7.41 (m, 2H), 7.31-7.35 (m, 1H), 5.32 (s, 1H), 3.89 (s, 3H), 3.72-3.73 (m, 2H), 3.51-3.52 (m, 2H), 2.58-2.61 (m, 1H), 2.47-2.49 (m, 2H), 2.33-2.37 (m, 1H).

### Example 106

### (R/S)-(4-(5-(4H-1,2,4-triazol-4-yl)benzo[d]oxazol-2-yl)pyridin-2-yl)(4-((2-(difluoromethyl)-2H-tetrazol-5-yl)(phenyl)methyl)piperazin-1-yl)methanone

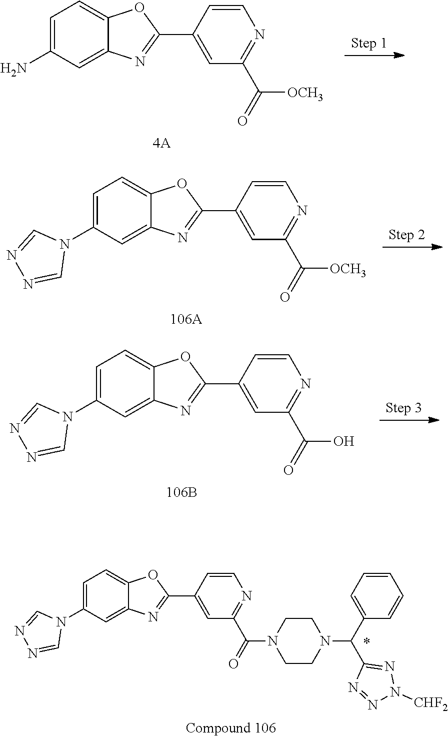 [SMILES: COC(=O)c1cc(-c2nc3cc(N)ccc3o2)ccn1 ; COC(=O)c1cc(-c2nc3cc(-n4cnnc4)ccc3o2)ccn1 ; O=C(O)c1cc(-c2nc3cc(-n4cnnc4)ccc3o2)ccn1 ; O=C(c1cc(-c2nc3cc(-n4cnnc4)ccc3o2)ccn1)N1CCN(C(c2ccccc2)c2nnn(C(F)F)n2)CC1]

Step 1:

### Methyl 4-(5-(4H-1,2,4-triazol-4-yl)benzo[d]oxazol-2-yl)picolinate (106A)

Compound 4a (500 mg, 1.64 mmol) and N,N′-bis(dimethylaminomethylene)hydrazine (349 mg, 2.46 mmol) were added to pyridine (10 mL), and the mixture was warmed to 95° C. and stirred for 17 h. The reaction was cooled to room temperature, and the reaction solution was concentrated and then separated and purified by silica gel column chromatography (DCM:MeOH=30:1) to obtain 106A (80 mg, 15%).

LC-MS (ESI): m/z=322.1[M+H]⁺.

Step 2:

### 4-(5-(4H-1,2,4-triazol-4-yl)benzo[d]oxazol-2-yl)picolinic Acid (106B)

Anhydrous methanol (10 mL) and NaOH (50 mg, 1.25 mmol, 1 mL) aqueous solution were successively added to compound 106A (80 mg, 0.25 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent and then obtain a crude product of 106B, which was directly used in the next reaction.

LC-MS (ESI): m/z=308.1 [M+H]⁺.

Step 3:

### (R/S)-(4-(5-(4H-1,2,4-triazol-4-yl)benzo[d]oxazol-2-yl)pyridin-2-yl)(4-((2-(difluoromethyl)-2H-tetrazol-5-yl)(phenyl)methyl)piperazin-1-yl)methanone (Compound 106)

At room temperature, DMF (8 mL), intermediate 7 (62 mg, 0.21 mmol), HATU (120 mg, 0.32 mmol) and DIPEA (81 mg, 0.63 mmol) were successively added to compound 106B (65 mg, 0.21 mmol). The reaction was stirred for 1 hour. The reaction solution was poured into water (50 mL) and extracted with ethyl acetate (20 mL×3). The organic phase was concentrated, and the residue was separated and purified by HPLC to obtain compound 106 (30 mg, 25%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.12 min.

LC-MS (ESI): m/z=584.3 [M+H]⁺.

¹H NMR (400 MHz, DMSO-d₆) δ 9.19 (s, 2H), 8.84-8.85 (m, 1H), 8.56 (t, 1H), 8.32 (d, 1H), 8.21-8.22 (m, 1H), 8.17-8.19 (m, 1H), 8.08 (d, 1H), 7.85-7.88 (m, 1H), 7.49-7.51 (m, 2H), 7.37-7.41 (m, 2H), 7.31-7.35 (m, 1H), 5.32 (s, 1H), 3.72-3.73 (m, 2H), 3.51-3.52 (m, 2H), 2.58-2.61 (m, 1H), 2.47-2.49 (m, 2H), 2.33-2.37 (m, 1H).

### Example 107

### (R/S)-(4-((2-(difluoromethyl)-2H-tetrazol-5-yl)(phenyl)methyl)piperazin-1-yl)(4-(6-(1-methyl-1H-imidazol-2-yl)benzo[d]oxazol-2-yl)pyridin-2-yl)methanone

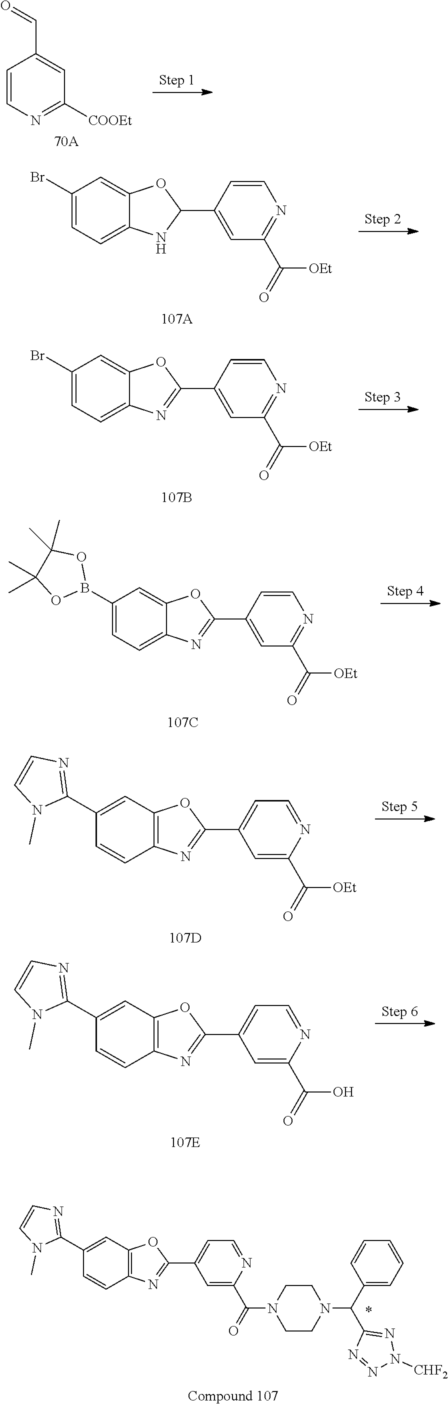

Step 1:

### Ethyl 4-(6-bromo-2,3-dihydrobenzo[d]oxazol-2-yl)picolinate (107A)

Compound 70A (1.0 g, 5.59 mmol) and 2-amino-5-bromophenol (1.05 g, 5.59 mmol) were added to ethanol (20 mL), and the mixture was warmed to 75° C. and stirred for 15 h. The reaction was cooled to room temperature and concentrated under reduced pressure, and the residue was separated and purified by silica gel column chromatography (PE:EA=1:1) to obtain 107A (1.10 g, 56%).

Step 2:

### Ethyl 4-(6-bromobenzo[d]oxazol-2-yl)picolinate (107B)

Compound 107A (710 mg, 2.04 mmol) was added to dichloromethane (300 mL), and then manganese dioxide (887 mg, 10.2 mmol) was added. The mixture was stirred at 25° C. overnight. The resulting solution was filtered; the filtrate was concentrated; and then the residue was separated and purified by silica gel column chromatography (PE:EA=1:1) to obtain 107B (660 mg, 94%).

LC-MS (ESI): m/z=347.1[M+H]⁺.

Step 3:

### Ethyl 4-(6-(4,4,5,5-tetramethyl-1,3,2-dioxaborolan-2-yl)benzo[d]oxazol-2-yl)picolinate (107C)

Compound 107B (500 mg, 1.44 mmol) was dissolved in dioxane (20 mL), and then bis(pinacolato)diboron (732 mg, 2.88 mmol), potassium acetate (423 mg, 4.32 mmol) and Pd(dppf)Cl₂ (102 mg, 0.14 mmol) were added. The mixture was warmed to 100° C. and stirred for 5 h under nitrogen protection. The reaction was cooled to room temperature, and the filtrate was concentrated and then separated and purified by silica gel column chromatography (PE:EA=1:1) to obtain 107C (500 mg, 88%).

LC-MS (ESI): m/z=395.2[M+H]⁺.

Step 4:

### Ethyl 4-(6-(1-methyl-1H-imidazol-2-yl)benzo[d]oxazol-2-yl)picolinate (107D)

Compound 107C (300 mg, 0.76 mmol) was added to a mixed solvent of dioxane (20 mL) and water (1 mL), then 2-bromo-1-methyl-1H-imidazole (367 mg, 2.28 mmol), potassium carbonate (210 mg, 1.52 mmol) and Pd(dppf)Cl₂ (58 mg, 0.08 mmol) were added. The mixture was warmed to 100° C. and stirred for 10 h under nitrogen protection. The reaction was cooled to room temperature, and the filtrate was concentrated and then separated and purified by silica gel column chromatography (PE:EA=1:3) to obtain 107D (150 mg, 57%).

LC-MS (ESI): m/z=349.1[M+H]⁺.

Step 5:

### 4-(6-(1-methyl-1H-imidazol-2-yl)benzo[d]oxazol-2-yl)picolinic Acid (107E)

Anhydrous methanol (8 mL) and NaOH (69 mg, 1.72 mmol, 0.5 mL) aqueous solution were successively added to compound 107D (150 mg, 0.43 mmol), and the mixture was stirred at 25° C. overnight. The reaction solution was adjusted to pH=2-3 by dropwise adding 1 N hydrochloric acid, concentrated under reduced pressure to remove the solvent and dried to obtain a crude product of compound 107E, which was directly used in the next reaction (180 mg).

LC-MS (ESI): m/z=321.1 [M+H]⁺.

Step 6:

### (R/S)-(4-((2-(difluoromethyl)-2H-tetrazol-5-yl)(phenyl)methyl)piperazin-1-yl)(4-(6-(1-methyl-1H-imidazol-2-yl)benzo[d]oxazol-2-yl)pyridin-2-yl)methanone (Compound 107)

At room temperature, DMF (8 mL), intermediate 7 (148 mg, 0.51 mmol), HATU (319 mg, 0.84 mmol) and DIPEA (289 mg, 2.24 mmol) were successively added to compound 107E (180 mg, 0.56 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 107 (35 mg, 10%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.12 min.

LC-MS (ESI): m/z=597.3 [M+H]⁺.

¹H NMR (400 MHz, DMSO-d₆) δ8.82-8.84 (m, 1H), 8.55 (t, 1H), 8.21-8.22 (m, 1H), 8.16-8.18 (m, 1H), 8.14-8.15 (m, 1H), 7.98 (d, 1H), 7.82-7.84 (m, 1H), 7.49-7.51 (m, 2H), 7.37-7.41 (m, 2H), 7.31-7.35 (m, 2H), 7.04 (d, 1H), 5.32 (s, 1H), 3.83 (s, 3H), 3.72-3.73 (m, 2H), 3.51-3.52 (m, 2H), 2.57-2.61 (m, 1H), 2.48-2.51 (m, 2H), 2.33-2.37 (m, 1H).

### Example 108

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-2-fluoro-2-methylpropanamide

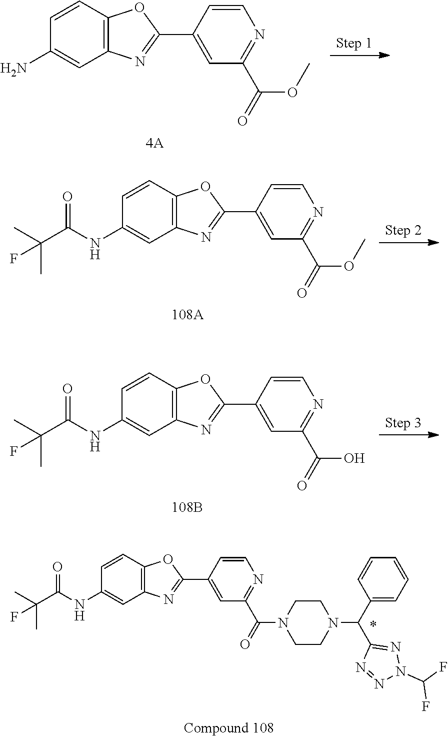

Step 1:

### Methyl 4-(5-(2-fluoro-2-methylpropanamido)benzo[d]oxazol-2-yl)picolinate (108A)

DMF (50 mL), 2-fluoro-2-methylpropanoic acid (395 mg, 3.72 mmol), HATU (2.1 g, 5.58 mmol) and DIEA (1.44 g, 11.16 mmol) were successively added to compound 4a (1.0 g, 3.72 mmol), and the mixture was stirred at room temperature for 5 h. The reaction was quenched by adding water, extracted 3 times with ethyl acetate and washed twice with saturated brine. The organic phase was dried and concentrated, and the residue was separated and purified by silica gel column chromatography (EA:PE=20%-40%) to obtain 108A (700 mg, 52%).

LC-MS (ESI): m/z=358.1[M+H]⁺.

Step 2:

### 4-(5-(2-fluoro-2-methylpropanamido)benzo[d]oxazol-2-yl)picolinic Acid (108B)

At room temperature, compound 108A (700 mg, 1.96 mmol) was dissolved in methanol (20 mL), and lithium hydroxide (700 mg) was dissolved in 20 mL of pure water. An aqueous solution of lithium hydroxide was added to the reaction solution; the mixture was stirred at 40° C. for 0.5 hours and then adjusted to pH 6-7 with 2 N hydrochloric acid; and the resulting solution was extracted with ethyl acetate (30 mL×3). The combined organic phase was dried over anhydrous sodium sulfate and concentrated under reduced pressure to obtain 108B (700 mg, 100%).

LC-MS (ESI): m/z=344.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-2-fluoro-2-methylpropanamide (Compound 108)

DMF (20 mL), intermediate 7 (336 mg, 1.02 mmol), HATU (570 mg, 1.5 mmol) and DIEA (387 mg, 3.0 mmol) were successively added to compound 108B (350 mg, 1.02 mmol), and the mixture was stirred at room temperature for 5 h. The reaction was quenched by adding water, extracted 3 times with ethyl acetate and washed twice with saturated brine. The organic phase was dried and concentrated to obtain a crude product compound, which was subjected to preparative chromatography to obtain compound 108 (200 mg, 32%). Preparative separation conditions: instrument: waters 2767 (preparative liquid phase chromatographic instrument); chromatographic column: XBridge@ Prep C18 (19 mm×250 mm); The sample was dissolved in DMF and filtered with a 0.45 m filter to prepare a sample solution. Preparative chromatography conditions: mobile phase A: acetonitrile; mobile phase B: water (containing 0.5% ammonia water); gradient elution: the content of mobile phase A rises from 40% to 75%; flow rate: 15 ml/min; elution time: 20 min; retention time: about 15 min;

LC-MS (ESI): m/z=620.2 [M+H]⁺.

¹H NMR (400 MHz, DMSO-d₆) δ 10.17 (s, 1H), 8.81 (d, 1H), 8.42-8.72 (m, 1H), 8.29 (d, 1H), 8.15-8.21 (m, 2H), 7.72-7.85 (m, 2H), 7.45-7.55 (m, 2H), 7.31-7.41 (m, 3H), 5.36 (s, 1H), 3.45-3.75 (m, 4H), 2.31-2.65 (m, 4H), 1.64 (s, 3H), 1.58 (s, 3H).

### Example 109: (R/S)—N-(2-(2-(2-(4-((2-(difluoromethyl)-2H-tetrazol-5-yl)(phenyl)methyl)piperazine-1-carbonyl)pyridin-4-yl)benzo[d]oxazol-5-yl)-2-(methyl-d3)propionamide

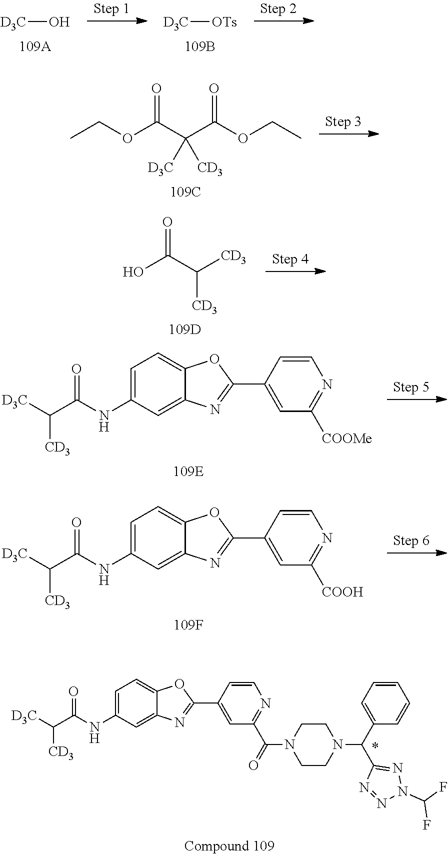

Step 1:

### methyl-d₃ 4-methylbenzenesulfonate (109B)

Compound 109A (1.0 g, 27.78 mmol) and p-toluenesulfonyl chloride (5.0 g, 26.32 mmol) were dissolved in 6 mL of tetrahydrofuran; 20% sodium hydroxide aqueous solution was added below 20° C.; and the mixture was stirred at room temperature for 16 hours. After the reaction was completed, the reaction solution was extracted 3 times with ethyl acetate. The organic phase was combined, dried and concentrated to obtain 109B (4 g, 76%).

Step 2:

### Diethyl 2,2-bis(methyl-d3)malonate (109C)

Compound diethyl malonate (678.8 mg, 4.24 mmol), cesium carbonate (3.45 g, 10.58 mmol), sodium iodide (126 mg, 0.84 mmol) and tetrabutylammonium bromide (270 mg, 0.84 mmol) were dissolved in 10 mL of N,N-dimethylformamide, and compound 109B (2.0 g, 10.58 mmol) dissolved in 1 mL of N,N-dimethylformamide was added. The mixture was stirred at room temperature for 16 hours. After the reaction was completed, water was added, and the resulting solution was extracted with ethyl acetate. The organic phase was combined and dried to obtain compound 109C (575 mg, 70%).

Step 3:

### 2-(methyl-d₃)propanoic-3,3,3-d₃ Acid (109D)

Compound 109C (1.15 g, 5.93 mmol) and sodium hydroxide (474 mg, 11.86 mmol) were dissolved in 1 mL of ethanol and 4 mL of water, and the mixture was heated to 75° C. and stirred for 2 hours. and distilled under reduced pressure to remove ethanol; 8 mL of concentrated hydrochloric acid was added to the reaction solution, and the resulting solution was heated to 130° C. in a sealed tube and reacted for 48 hours. After the reaction was completed, the reaction solution was extracted twice with dichloromethane. The organic phase was dried and concentrated to obtain compound 109D (300 mg, 54%).

Step 4:

### Methyl 4-(5-(2-(methyl-d3)propanamido-3,3,3-d3)benzo[d]oxazol-2-yl)picolinate (109E)

Methyl 4-(5-aminobenzo[d]oxazol-2-yl)picolinate (500 mg, 1.86 mmol) was dissolved in 10 mL of N,N-dimethylformamide; HATU (1.06 g, 2.79 mmol) and N,N-diisopropylethylamine (720 mg, 5.58 mmol) were added; and compound 109D (262 mg, 2.79 mmol) was added. The mixture was stirred at room temperature and reacted for 2 hours. After TLC showed that the reaction was completed, water was added, and the resulting solution was extracted with ethyl acetate. The organic phase was dried and concentrated to obtain a crude product of 109E.

LC-MS (ESI): m/z=346.2 [M+H]⁺.

Step 5:

### 4-(5-(2-(methyl-d3)propanamido-3,3,3-d3)benzo[d]oxazol-2-yl)picolinic Acid (109F)

The crude product of 109E was dissolved in 50 mL of methanol, and an aqueous solution of lithium hydroxide (223 mg, 9.3 mmol) was added. The reaction was stirred at room temperature for 1 hour. After TLC showed that the reaction was completed, the reaction solution was concentrated, diluted by adding water and adjusted to pH 3 with 2 N dilute hydrochloric acid solution. The solid was precipitated in the reaction solution and filtered by suction, and the filter cake was dried to obtain 109F (525 mg, 87%).

LC-MS (ESI): m/z=332.1 [M+H]⁺.

Step 6:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-2-(methyl-d3)propanamide (Compound 109)

109F (300 mg, 0.9 mmol) was dissolved in 10 mL of N,N-dimethylformamide; HATU (517 mg, 1.36 mmol), N,N-diisopropylethylamine (0.5 mL, 2.7 mmol) and intermediate 7 (357 mg, 0.9 mmol) were added; and the mixture was reacted at room temperature for 2 hours. After TLC showed that the reaction was completed, the reaction solution was extracted with ethyl acetate and water and washed twice with saturated brine. The organic phase was dried and concentrated to obtain a crude product of compound 109, which was subjected to preparative chromatography to obtain compound 109 (51 mg, 9%). Preparation method: instrument: waters 2767 (preparative liquid phase chromatographic instrument); chromatographic column: SunFire@ Prep C18 (19 mm×250 mm); The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.66 min.

LC-MS (ESI): m/z=608.2 [M+H]⁺.

¹H NMR (400 MHz, CD₃Cl) δ 8.72 (d, 1H), 8.36 (s, 1H), 8.00-8.09 (m, 2H), 7.56-7.62 (m, 1H), 7.52-7.55 (m, 4H), 7.36-7.38 (m, 3H), 5.19 (s, 1H), 3.94 (s, 2H), 3.73 (s, 2H), 2.52-2.79 (m, 5H).

### Example 110

### (R/S)-(4-((2-(difluoromethyl)-2H-tetrazol-5-yl)(phenyl)methyl)piperazin-1-yl)(4-(5-(thiazol-4-yl)benzo[d]oxazol-2-yl)pyridin-2-yl)methanone

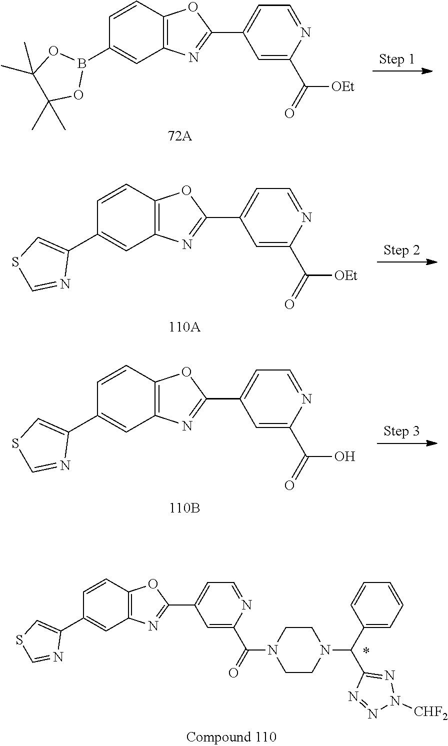 [SMILES: CCOC(=O)c1cc(-c2nc3cc(B4OC(C)(C)C(C)(C)O4)ccc3o2)ccn1 ; CCOC(=O)c1cc(-c2nc3cc(-c4cscn4)ccc3o2)ccn1 ; O=C(O)c1cc(-c2nc3cc(-c4cscn4)ccc3o2)ccn1 ; O=C(c1cc(-c2nc3cc(-c4cscn4)ccc3o2)ccn1)N1CCN(C(c2ccccc2)c2nnn(C(F)F)n2)CC1]

Step 1:

### Ethyl 4-(5-(thiazol-4-yl)benzo[d]oxazol-2-yl)picolinate (110A)

4-bromothiazole (0.43 g, 2.63 mmol), methyl 4-(5-(4,4,5,5-tetramethyl-1,3,2-dioxaborolan-2-yl)benzo[d]oxazol-2-yl)picolinate (0.50 g, 1.32 mmol), Pd(dppf)Cl₂ (0.19 g, 2.63 mmol) and potassium carbonate (0.55 g, 3.95 mmol) were added to a 250 ml three-necked flask, and then the mixture was dissolved by adding DMF (10 mL) and reacted at 80° C. for 3 h. The reaction was quenched by adding water, and the reaction solution was extracted with ethyl acetate, washed with saturated brine, dried over anhydrous sodium sulfate and concentrated, and the residue was purified by column chromatography (eluent (PE:EA=1:1)) to obtain the target compound (0.17 g, 38%).

LC-MS (ESI): m/z=352.1 [M+H]⁺.

Step 2:

### 4-(5-(thiazol-4-yl)benzo[d]oxazol-2-yl)picolinic Acid (110B)

To a 100 ml round bottom flask, ethyl 4-(5-(thiazol-4-yl)benzo[d]oxazol-2-yl)picolinate (0.17 g, 0.50 mmol) and LiOH (0.04 g, 1.52 mmol) were added and dissolved in a mixed solvent of THF/H₂O (4:1). The mixture was reacted at room temperature overnight. The reaction solution was extracted with ethyl acetate. The aqueous phase was collected, adjusted to pH=2 with dilute hydrochloric acid, extracted with DCM, washed with saturated brine, dried over anhydrous sodium sulfate and concentrated to obtain the target compound (0.12 g, 74%).

LC-MS (ESI): m/z=324.1 [M+H]⁺.

Step 3:

### (R/S)-(4-((2-(difluoromethyl)-2H-tetrazol-5-yl)(phenyl)methyl)piperazin-1-yl)(4-(5-(thiazol-4-yl)benzo[d]oxazol-2-yl)pyridin-2-yl)methanone (Compound 110)

To a 100 ml round bottom flask, 4-(5-(thiazol-4-yl)benzo[d]oxazol-2-yl)picolinic acid (0.12 g, 0.37 mmol), intermediate 7 (0.11 g, 0.37 mmol), HATU (0.21 g, 0.56 mmol) and DIEA (0.14 g, 1.11 mmol) were added and dissolved in DMF (10 mL). The mixture was reacted at room temperature overnight. The reaction was diluted by adding water and extracted with ethyl acetate, and the organic phase was collected, washed with saturated brine, dried over anhydrous sodium sulfate and separated and purified by prep-HPLC to obtain compound 110 (80 mg, 36%). Preparation conditions: instrument: waters 2767 (preparative liquid phase chromatographic instrument); chromatographic column: XBridge C18 5 μm, 19×250 mm. The sample was dissolved in acetonitrile and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 5% to 50%; time: 15 min; flow rate: 12 ml/min. Components with retention time of 9.5 min were used

LC-MS (ESI): m/z=600.3 [M+H]⁺.

¹H NMR (400 MHz, DMSO-d₆) δ9.25 (d, J=4.0 Hz, 1H), 8.83 (d, J=4.0 Hz, 1H), 8.70-8.42 (m, 2H), 8.32 (s, 1H), 8.21-8.16 (m, 3H), 7.93 (d, J=8.0 Hz, 1H), 7.50 (d, J=8.0 Hz, 2H), 7.41-7.31 (m, 3H), 5.32 (s, 1H), 3.73-3.71 (m, 2H), 3.52-3.50 (m, 2H), 2.67-2.58 (m, 2H), 2.39-2.33 (m, 2H).

### Example 111

### (1S,2S)—N-(2-(2-(4-((R/S)-(2-(difluoromethyl)-2H-tetrazol-5-yl)(phenyl)methyl)piperazine-1-carbonyl)pyridin-4-yl)benzofuran-5-yl)-2-fluorocyclopropane-1-carboxamide

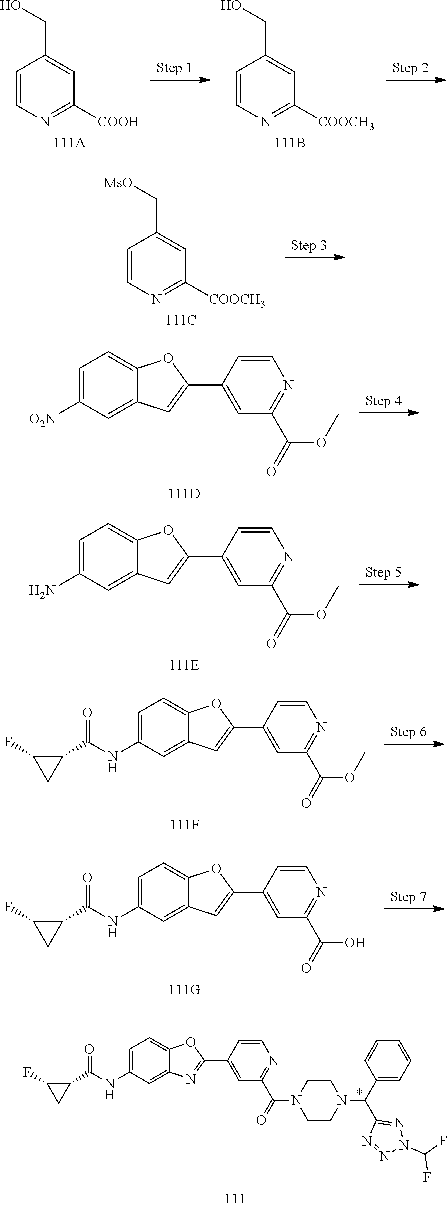

Step 1:

### Methyl-4-(hydroxymethyl)picolinate (111B)

Compound 111A (5 g, 32.6 mmol) was added to a single-necked flask, and thionyl chloride (13.22 g, 98 mmol) was added dropwise under an ice bath. The reaction was stirred at 80° C. for 4 hours. The reaction was cooled to room temperature and concentrated under reduced pressure to obtain a crude product. 100 mL of ethyl acetate was added to the crude product. The organic phase was washed with distilled water (100 mL×2) and concentrated under reduced pressure to obtain compound 111B (5.3 g, 97.1%).

LC-MS (ESI): m/z=168.1[M+H]⁺.

Step 2:

### Methyl-4-(((methylsulfonyl)oxy)methyl)picolinate (111C)

Compound 111B (1.5 g, 8.97 mmol), dichloromethane (5 mL) and triethylamine (1.22 g, 12 mmol) were successively added to a single-necked flask, and methyl sulfonyl chloride (1.34 g, 11.7 mmol) was added dropwise under an ice bath. After the dropwise addition was completed, the reaction was stirred at room temperature for 1 h. 100 mL of distilled water was added. The resulting solution was extracted with dichloromethane (50 mL×2). The combined organic phase was dried over anhydrous sodium sulfate and concentrated under reduced pressure, and then the residue was separated and purified by silica gel column chromatography (eluent: EA/PE=1/10) to obtain compound 111C (2 g, 90.9%).

LC-MS (ESI): m/z=246.1[M+H]⁺.

Step 3:

### Methyl-4-(5-nitrobenzofuran-2-yl)picolinate (111D)

Under nitrogen protection, 111C (700 mg, 2.85 mmol), DMF (15 mL), 2-hydroxyl-5-nitro-benzaldehyde (524 mg, 3.14 mmol) and K₂CO₃ (473 mg, 3.42 mmol) were successively added to a single-necked flask, and the mixture was refluxed with stirring at 85° C. overnight. The reaction was quenched by adding saturated aqueous sodium bicarbonate solution (30 mL), extracted with ethyl acetate (50 mL×1) and allowed to stand for phase separation. The aqueous phase was washed with ethyl acetate (100 mL×2). The combined organic phase was dried over anhydrous sodium sulfate and concentrated under reduced pressure to obtain a crude product from the residue, and the crude product was separated by column chromatography (eluent: EA/PE=1/2) to obtain compound 111D (500 mg, 58.7%).

LC-MS (ESI): m/z=299.1[M+H]⁺.

Step 4:

### Methyl-4-(5-aminobenzofuran-2-yl)picolinate (111E)

111D (500 mg, 1.68 mmol), methanol (40 mL) and palladium on carbon (100 mg) were successively added to a 100 mL single-necked flask. The reaction was stirred at room temperature under hydrogen atmosphere for 2 hours. After TLC showed that the reaction was completed, palladium on carbon was filtered off through celite. The organic phase was concentrated under reduced pressure to obtain a crude product of 111E (430 mg, 95.6%) from the residue. The crude product was directly used in the next reaction without separation by column chromatography.

LC-MS (ESI): m/z=269.2[M+H]⁺.

Step 5:

### methyl-4-(5-((1S,2S)-2-fluorocyclopropane-1-carboxamido)benzofuran-2-yl)picolinate (111F)

Under nitrogen protection, (1S,2S)-2-fluorocycloprop-1-carboxylic acid (200 mg, 1.92 mmol), DMF (10 mL), DIPEA (383 mg, 2.88 mmol), HATU (800 mg, 2.11 mmol) and compound 111E (530 mg, 1.98 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (100 mL×2). The combined organic phase was dried over anhydrous sodium sulfate and concentrated under reduced pressure to obtain a crude product from the residue, and the crude product was separated by column chromatography (eluent: EA/PE=1/2) to obtain compound 111F (500 mg, 73.43%).

LC-MS (ESI): m/z=355.2[M+H]⁺.

Step 6:

### 4-(5-((1S,2S)-2-fluorocyclopropane-1-carboxamido)benzofuran-2-yl)picolinic acid (111G)

Under nitrogen protection, compound 111F (500 mg, 1.41 mmol), tetrahydrofuran (2 mL), methanol (5 ml), distilled water (2 mL) and lithium hydroxide monohydrate (300 mg, 7 mmol) were successively added to a single-necked flask, and the mixture was stirred at room temperature for 3 h. The reaction was quenched by adding dilute hydrochloric acid and extracted with dichloromethane (50 mL×2). The combined organic phase was dried over anhydrous sodium sulfate and concentrated under reduced pressure to obtain a crude product of compound 111G (430 mg, 93.7%) from the residue. The crude product can be directly used in the next reaction.

LC-MS (ESI): m/z=355.2[M+H]⁺.

Step 7:

### (1S,2S)—N-(2-(2-(4-((R/S)-(2-(difluoromethyl)-2H-tetrazol-5-yl)(phenyl)methyl)piperazine-1-carbonyl)pyridin-4-yl)benzofuran-5-yl)-2-fluorocyclopropane-1-carboxamide (Compound 111)

Under nitrogen protection, compound 111 g (100 mg, 0.3 mmol), DMF (10 mL), HATU (137 mg, 0.36 mmol), DIPEA (58 mg, 0.45 mmol) and intermediate 7 (106 mg, 0.36 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (100 mL×2). The combined organic phase was dried over anhydrous sodium sulfate and concentrated under reduced pressure to obtain a crude product from the residue, and the crude product was separated by preparative high-performance liquid chromatography to obtain compound 111 as a light yellow solid (80 mg, 44.15%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.36 min.

¹H NMR (400 MHz, CDCl₃) δ 8.58 (d, J=5.2 Hz, 1H), 8.00 (s, 2H), 7.89-7.55 (m, 4H), 7.55-7.34 (m, 3H), 7.32-7.27 (m, 1H), 7.20 (s, 1H), 5.40 (s, 1H), 5.03-4.55 (m, 2H), 4.03-3.88 (m, 4H), 3.21-2.45 (m, 4H), 2.00 (s, 1H), 1.96-1.73 (m, 2H), 1.31-1.12 (m, 2H).

LC-MS (ESI): m/z=617.2 [M+H]⁺.

### Example 112

### (R/S)-(4-((2-(difluoromethyl)-2H-tetrazol-5-yl)(phenyl)methyl)piperazin-1-yl)(4-(5-(pyrazin-2-yl)benzo[d]oxazol-2-yl)pyridin-2-yl)methanone

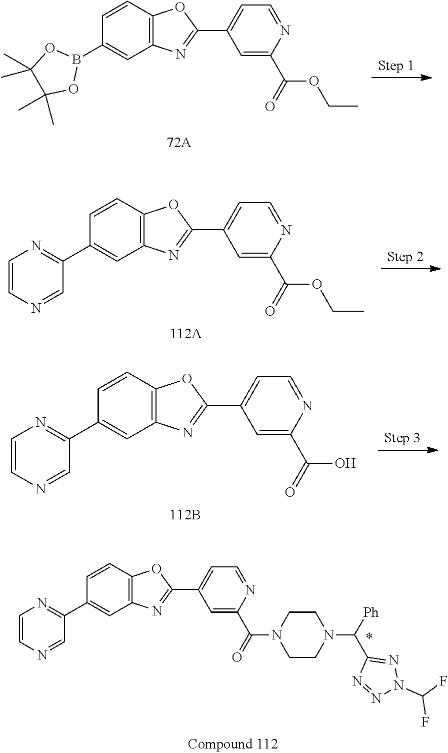 [SMILES: CCOC(=O)c1cc(-c2nc3cc(B4OC(C)(C)C(C)(C)O4)ccc3o2)ccn1 ; CCOC(=O)c1cc(-c2nc3cc(-c4cnccn4)ccc3o2)ccn1 ; O=C(O)c1cc(-c2nc3cc(-c4cnccn4)ccc3o2)ccn1 ; O=C(c1cc(-c2nc3cc(-c4cnccn4)ccc3o2)ccn1)N1CCN(C(c2ccccc2)c2nnn(C(F)F)n2)CC1]

Step 1:

### Ethyl 4-(5-(pyrazin-2-yl)benzo[d]oxazol-2-yl)picolinate (112A)

Compound 72A (600 mg, 1.52 mmol), 2-bromopyrazine (477 mg, 3 mmol), Pd(PPh₃)₄ (526 mg, 0.46 mmol) and potassium carbonate (630 mg, 4.56 mmol) were added to a mixed system of dioxane (10 mL) and water (1 ml). After N₂ replacement, the mixture was reacted at 100° C. for 3 h. After completion of the reaction, the reaction mixture was cooled to room temperature and filtered. The filtrate was concentrated under reduced pressure, and then the residue was separated and purified by column chromatography (eluent: PE/EA=1/3) to obtain 112A (280 mg, 53%).

LC-MS (ESI): m/z=347.1 [M+H]⁺.

Step 2:

### 4-(5-(pyrazin-2-yl)benzo[d]oxazol-2-yl)picolinic Acid (112B)

Compound 112A (280 mg, 0.8 mmol) and sodium hydroxide (200 mg, 4.8 mmol) were added to a mixed system of methanol (5 mL) and water (2 ml), and the mixture was reacted at room temperature for 2 hours. The reaction solution was adjusted to pH=4-5 with dilute hydrochloric acid, and the reaction solution was directly concentrated under reduced pressure and then used in the next reaction.

LC-MS (ESI): m/z=319.1 [M+H]⁺.

Step 3:

### (R/S)-(4-((2-(difluoromethyl)-2H-tetrazol-5-yl)(phenyl)methyl)piperazin-1-yl)(4-(5-(pyrazin-2-yl)benzo[d]oxazol-2-yl)pyridin-2-yl)methanone (Compound 112)

112B was dissolved in N,N-dimethylformamide (5 mL). HATU (456 mg, 1.2 mmol), DIPEA (310 mg, 2.4 mmol) and intermediate 7 (230 mg, 0.8 mmol) were added. After the addition, the mixture was stirred for 1 hour at room temperature, and then the reaction was quenched by adding water (20 mL). The resulting solution was extracted twice with ethyl acetate (20 mL×3). The organic phase was combined, dried over anhydrous sodium sulfate and concentrated under reduced pressure, and then the residue was separated by column chromatography (eluent: MeOH/DCM=1/20) to obtain compound 112 (100 mg, 21%).

¹H NMR (400 MHz, MeOD) δ 9.21 (d, 1H), 8.83-8.81 (m, 1H), 8.72-8.70 (m, 1H), 8.57-8.55 (m, 2H), 8.37-8.34 (m, 1H), 8.31-8.01 (m, 3H), 7.89 (d, 1H), 7.56-7.51 (m, 2H), 7.3-7.30 (m, 3H), 5.20 (s, 1H), 3.86 (t, 2H), 3.60 (t, 2H), 2.78-2.67 (m, 1H), 2.66-2.51 (m, 2H), 2.49-2.40 (m, 1H).

LC-MS (ESI): m/z=595.2[M+H]⁺.

### Example 113

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-N-methylacetamide

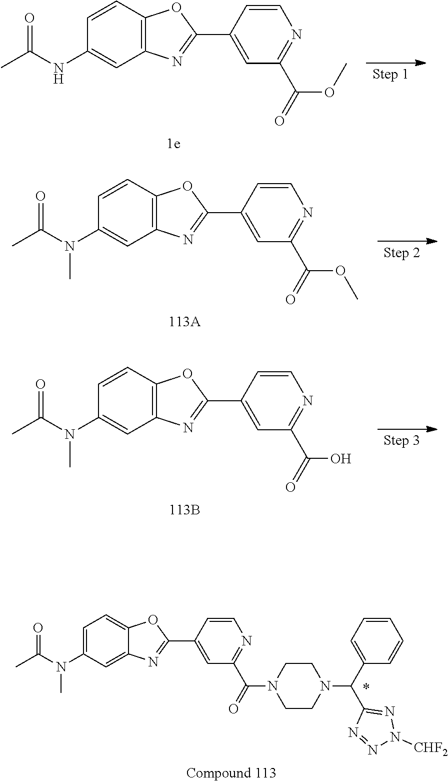

Step 1:

### Methyl 4-(5-(N-methylacetamido)benzo[d]oxazol-2-yl)picolinate (113A)

Compound 1e (1.8 g, 5.8 mmol) was added to DMF (120 mL). NaH (463 mg, 11.6 mmol) was added. The mixture was stirred at 50° C. for 10 min; iodomethane (4.1 g, 29 mmol) was added; and then the resulting mixture was reacted for another 3 h. The reaction solution was cooled to room temperature, then poured into 500 ml of water and filtered. The filtered solid was washed with water, and then subjected to column chromatography to obtain compound 113A (1.2 g, 60%).

LC-MS (ESI): m/z=326.1[M+H]⁺.

Step 2:

### 4-(5-(N-methylacetamido)benzo[d]oxazol-2-yl)picolinic Acid (113B)

Compound 113A (500 mg, 1.54 mmol) and sodium hydroxide (307 mg, 7.7 mmol) were added to a mixed system of methanol (5 mL) and water (2 ml), and the mixture was reacted at room temperature for 2 hours. The reaction solution was adjusted to pH=1-2 with dilute hydrochloric acid, and a small amount of acetone was added to the reaction solution. The mixture was filtered, and the filtered solid was washed with water to obtain compound 113B (420 mg).

LC-MS (ESI): m/z=312.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)-N-methylacetamide (Compound 113)

Intermediate 7 (76 mg, 0.26 mmol) and 113B (100 mg, 0.32 mmol) were dissolved in N,N-dimethylformamide (5 mL). HATU (146 mg, 0.38 mmol) and DIPEA (124 mg, 1 mmol) were added. After the addition, the mixture was stirred for 1 hour at room temperature, and then the reaction was quenched by adding water (30 mL). The resulting solution was extracted twice with ethyl acetate (30 mL×3). The organic phase was combined, dried over anhydrous sodium sulfate and concentrated under reduced pressure, and then the residue was separated by column chromatography (eluent: MeOH/DCM=1/20) to obtain compound 113 (110 mg, 72%).

1H NMR (400 MHz, CDCl₃) δ 8.75 (d, 1H), 8.43 (s, 1H), 8.12-8.11 (m, 1H), 7.78-7.47 (m, 5H), 7.41-7.27 (m, 4H), 5.17 (s, 1H), 3.96-3.94 (m, 2H), 3.75-3.73 (m, 2H), 3.33 (s, 3H), 2.76-2.48 (m, 4H), 1.90 (s, 3H).

LC-MS (ESI): m/z=588.2[M+H]⁺.

### Example 114

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl) Deuteratedacetamide

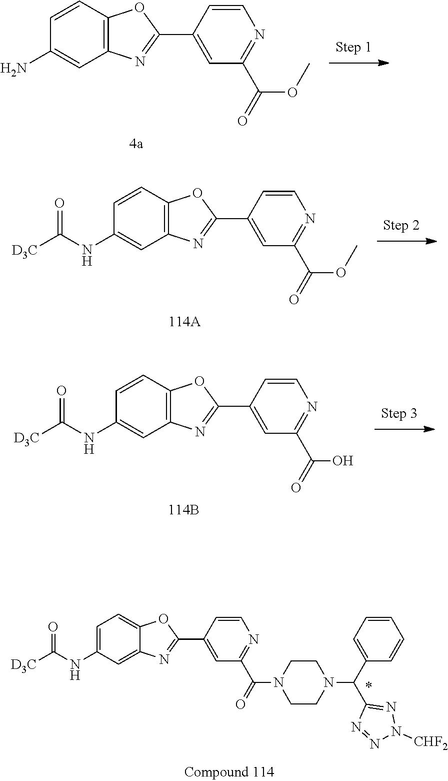 [SMILES: COC(=O)c1cc(-c2nc3cc(N)ccc3o2)ccn1 ; [2H]C([2H])([2H])C(=O)Nc1ccc2oc(-c3ccnc(C(=O)OC)c3)nc2c1 ; [2H]C([2H])([2H])C(=O)Nc1ccc2oc(-c3ccnc(C(=O)O)c3)nc2c1 ; [2H]C([2H])([2H])C(=O)Nc1ccc2oc(-c3ccnc(C(=O)N4CCN(C(c5ccccc5)c5nnn(C(F)F)n5)CC4)c3)nc2c1]

Step 1:

### Methyl 4-(5-deuterated acetamidobenzo[d]oxazol-2-yl)picolinate (114A)

Deuterated acetic acid (250 mg, 3.92 mmol) and 4a (1 g, 3.71 mmol) were dissolved in N,N-dimethylformamide (10 mL). HATU (1.8 g, 4.9 mmol) and DIPEA (1.68 g, 13 mmol) were added. After the addition, the mixture was stirred at room temperature for 1 hour. The reaction was quenched by adding water (50 mL) and directly filtered. The filtered solid was washed with water and dried under reduced pressure to obtain compound 114A (700 mg, 68%).

LC-MS (ESI): m/z=315.1 [M+H]⁺.

Step 2:

### 4-(5-deuterated acetamidobenzo[d]oxazol-2-yl)picolinic Acid (114B)

Compound 114A (700 mg, 2.23 mmol) and sodium hydroxide (446 mg, 11 mmol) were added to a mixed system of methanol (10 mL) and water (3 ml), and the mixture was reacted at room temperature for 2 hours. The reaction solution was adjusted to pH=1-2 with dilute hydrochloric acid, and a small amount of acetone was added to the reaction solution. The mixture was filtered, and the filtered solid was washed with water to obtain compound 114B (600 mg, 90%).

LC-MS (ESI): m/z=301.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl) Deuterated Acetamide (Compound 114)

Intermediate 7 (200 mg, 0.67 mmol) and 114B (200 mg, 0.67 mmol) were dissolved in N,N-dimethylformamide (5 mL). HATU (380 mg, 1 mmol) and DIPEA (258 mg, 2 mmol) were added. After the addition, the mixture was stirred for 1 hour at room temperature, and then the reaction was quenched by adding water (30 mL). The resulting solution was extracted twice with ethyl acetate (30 mL×3). The organic phase was combined, dried over anhydrous sodium sulfate and concentrated under reduced pressure, and then the residue was separated by column chromatography (eluent: MeOH/DCM=1/20) to obtain compound 114 (120 mg, 31%).

¹H NMR (400 MHz, CDCl 3) δ 8.70 (d, 1H), 8.25 (s, 1H), 8.04-8.03 (m, 1H), 7.94 (s, 1H), 7.83 (s, 1H), 7.76-7.53 (m, 3H), 7.49-7.31 (m, 5H), 5.17 (s, 1H), 3.93-3.91 (m, 2H), 3.70-3.68 (m, 2H), 2.76-2.47 (m, 4H).

LC-MS (ESI): m/z=577.2[M+H]⁺.

### Example 115

### (R/S)-(4-((2-(difluoromethyl)-2H-tetrazol-5-yl)(phenyl)methyl)piperazin-1-yl)(4-(5-(isothiazol-5-yl)benzo[d]oxazol-2-yl)pyridin-2-yl)methanone

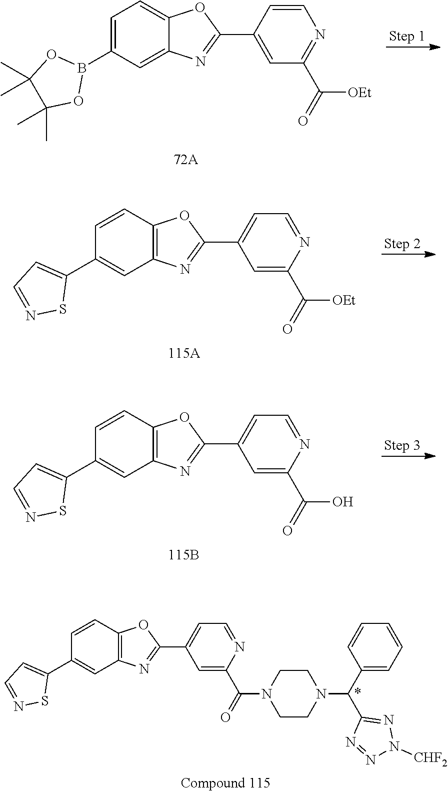 [SMILES: CCOC(=O)c1cc(-c2nc3cc(B4OC(C)(C)C(C)(C)O4)ccc3o2)ccn1 ; CCOC(=O)c1cc(-c2nc3cc(-c4ccns4)ccc3o2)ccn1 ; O=C(O)c1cc(-c2nc3cc(-c4ccns4)ccc3o2)ccn1 ; O=C(c1cc(-c2nc3cc(-c4ccns4)ccc3o2)ccn1)N1CCN(C(c2ccccc2)c2nnn(C(F)F)n2)CC1]

Step 1:

### Ethyl 4-(5-(isothiazol-5-yl)benzo[d]oxazol-2-yl)picolinate (115A)

Compound 72A (400 mg, 1.02 mmol) was added to a mixed solvent of dioxane (10 mL) and water (1 mL), and then 5-bromoisothiazole (334 mg, 2.04 mmol), potassium carbonate (282 mg, 2.04 mmol) and Pd(dppf)Cl₂ (74 mg, 0.10 mmol) were added. The mixture was warmed to 100° C. and stirred for 10 h under nitrogen protection. The reaction was cooled to room temperature, and the filtrate was concentrated and then separated and purified by silica gel column chromatography (DCM:MeOH=20:1) to obtain 115A (160 mg, 45%).

LC-MS (ESI): m/z=352.1[M+H]⁺.

Step 2:

### 4-(5-(isothiazol-5-yl)benzo[d]oxazol-2-yl)picolinic Acid (115B)

Anhydrous methanol (8 mL) and NaOH (91 mg, 2.28 mmol, 1 mL) aqueous solution were successively added to compound 115A (160 mg, 0.46 mmol), and the mixture was stirred at room temperature overnight. The reaction solution was adjusted to pH=2-3 by dropwise adding 1 N hydrochloric acid and concentrated under reduced pressure to remove the solvent. A small amount of water (1 mL) was added, and the resulting mixture was stirred and then filtered. The filter cake was dried to obtain a crude product of compound 115B, which was directly used in the next reaction (120 mg, 81%).

LC-MS (ESI): m/z=324.1 [M+H]⁺.

Step 3:

### (R/S)-(4-((2-(difluoromethyl)-2H-tetrazol-5-yl)(phenyl)methyl)piperazin-1-yl)(4-(5-(isothiazol-5-yl)benzo[d]oxazol-2-yl)pyridin-2-yl)methanone

At room temperature, DMF (8 mL), intermediate 7 (109 mg, 0.37 mmol), HATU (213 mg, 0.56 mmol) and DIPEA (143 mg, 1.11 mmol) were successively added to compound 115B (120 mg, 0.37 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 115 (15 mg, 7%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.36 min.

LC-MS (ESI): m/z=600.3 [M+H]⁺.

¹H NMR (400 MHz, DMSO-d₆) δ8.83-8.84 (m, 1H), 8.64 (d, 1H), 8.56 (t, 1H), 8.34-8.35 (m, 1H), 8.21-8.23 (m, 1H), 8.17-8.18 (m, 1H), 7.99 (d, 1H), 7.93 (d, 1H), 7.89-7.91 (m, 1H), 7.49-7.50 (m, 2H), 7.37-7.41 (m, 2H), 7.31-7.35 (m, 1H), 5.32 (s, 1H), 3.72-3.73 (m, 2H), 3.50-3.51 (m, 2H), 2.58-2.61 (m, 1H), 2.47-2.50 (m, 2H), 2.33-2.37 (m, 1H).

### Example 116

### (R/S)-(4-((2-(difluoromethyl)-2H-tetrazol-5-yl)(phenyl)methyl)piperazin-1-yl)(4-(5-(2-methyl-2H-tetrazol-5-yl)benzo[d]oxazol-2-yl)pyridin-2-yl)methanone

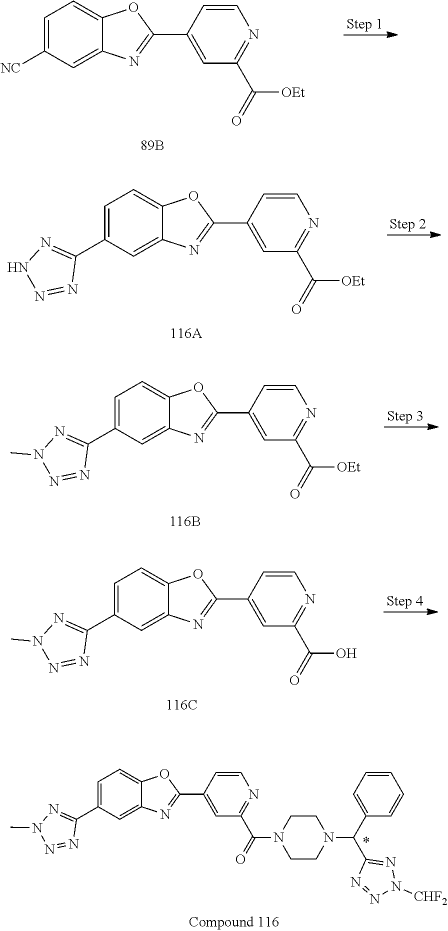 [SMILES: CCOC(=O)c1cc(-c2nc3cc(C#N)ccc3o2)ccn1 ; CCOC(=O)c1cc(-c2nc3cc(-c4nn[nH]n4)ccc3o2)ccn1 ; CCOC(=O)c1cc(-c2nc3cc(-c4nnn(C)n4)ccc3o2)ccn1 ; Cn1nnc(-c2ccc3oc(-c4ccnc(C(=O)O)c4)nc3c2)n1 ; Cn1nnc(-c2ccc3oc(-c4ccnc(C(=O)N5CCN(C(c6ccccc6)c6nnn(C(F)F)n6)CC5)c4)nc3c2)n1]

Step 1:

### Ethyl 4-(5-(2H-tetrazol-5-yl)benzo[d]oxazol-2-yl)picolinate (116A)

Compound 89B (350 mg, 1.19 mmol) and ammonium chloride (192 mg, 3.58 mmol) were added to anhydrous DMF (20 mL), and then sodium azide (233 mg, 3.58 mmol) was added. The mixture was warmed to 100° C. and stirred for 15 h. The reaction was cooled to room temperature and adjusted to pH 2-3 with 1 N hydrochloric acid. The reaction solution was poured into water (50 mL) and extracted with ethyl acetate (30 mL×4). The organic phase was concentrated, and the residue was separated and purified by silica gel column chromatography (DCM:MeOH=10:1) to obtain compound 116A (80 mg, 20%).

LC-MS (ESI): m/z=337.1[M+H]⁺.

Step 2:

### Ethyl 4-(5-(2-methyl-2H-tetrazol-5-yl)benzo[d]oxazol-2-yl)picolinate (116B)

Compound 116A (80 mg, 0.24 mmol) was added to a mixed solvent of methanol (4 mL) and tetrahydrofuran (1 mL), and then trimethylsilyldiazomethane (109 mg, 0.96 mmol) was added. The mixture was stirred at 25° C. for 10 hours. The reaction solution was separated and purified by silica gel column chromatography (DCM MeOH=20:1) to obtain 116B (40 mg, 48%).

LC-MS (ESI): m/z=351.1[M+H]⁺.

Step 3:

### 4-(5-(2-methyl-2H-tetrazol-5-yl)benzo[d]oxazol-2-yl)picolinic Acid (116C)

Anhydrous methanol (3 mL) and NaOH (18 mg, 0.44 mmol, 0.5 mL) aqueous solution were successively added to compound 116B (40 mg, 0.11 mmol), and the mixture was stirred at room temperature overnight. The reaction solution was adjusted to pH 2-3 by dropwise adding 1 N hydrochloric acid and concentrated under reduced pressure to obtain a crude product of compound 116C (56 mg, 100%), which was directly used in the next reaction.

LC-MS (ESI): m/z=323.1 [M+H]⁺.

Step 4:

### (R/S)-(4-((2-(difluoromethyl)-2H-tetrazol-5-yl)(phenyl)methyl)piperazin-1-yl)(4-(5-(2-methyl-2H-tetrazol-5-yl)benzo[d]oxazol-2-yl)pyridin-2-yl)methanone

At room temperature, DMF (8 mL), intermediate 7 (50 mg, 0.17 mmol), HATU (97 mg, 0.26 mmol) and DIPEA (66 mg, 0.51 mmol) were successively added to compound 116C (56 mg, 0.17 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 116 (8 mg, 8%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.80 min.

LC-MS (ESI): m/z=598.3 [M+H]⁺.

¹H NMR (400 MHz, DMSO-d₆) δ8.83-8.85 (m, 1H), 8.56 (t, 1H), 8.48-8.49 (m, 1H), 8.21-8.24 (m, 2H), 8.18-8.20 (m, 1H), 8.04-8.07 (m, 1H), 7.49-7.50 (m, 2H), 7.37-7.41 (m, 2H), 7.31-7.35 (m, 1H), 5.32 (s, 1H), 4.46 (s, 3H), 3.72-3.73 (m, 2H), 3.50-3.51 (m, 2H), 2.58-2.61 (m, 1H), 2.47-2.50 (m, 2H), 2.33-2.37 (m, 1H).

### Example 117

### (1S,2S)-2-fluoro-N-(2-(2-(4-((2-methyl-2H-tetrazol-5-yl)(phenyl)amino)piperidine-1-carbonyl)pyridin-4-yl)benzo[d]oxazol-5-yl)cyclopropane-1-carboxamide

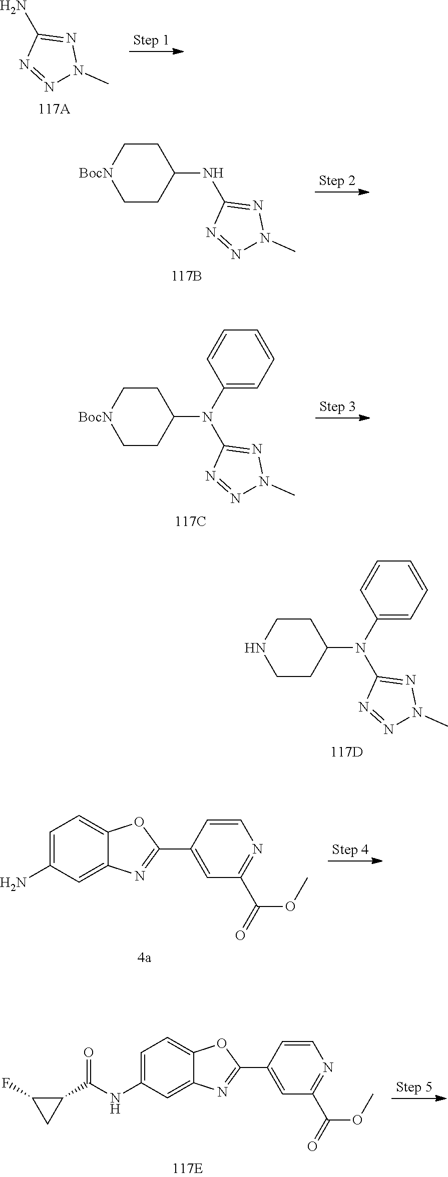

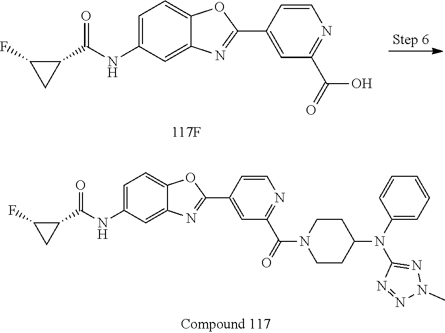 [SMILES: O=C(O)c1cc(-c2nc3cc(NC(=O)[C@@H]4C[C@@H]4F)ccc3o2)ccn1 ; Cn1nnc(N(c2ccccc2)C2CCN(C(=O)c3cc(-c4nc5cc(NC(=O)[C@@H]6C[C@@H]6F)ccc5o4)ccn3)CC2)n1]

Step 1:

### tert-butyl-4-((2-methyl-2H-tetrazol-5-yl)amino)piperidine-1-carboxylate (117B)

To a single-necked flask, compound 117A (2 g, 20.2 mmol), N-Boc-4-piperidone (4.42 g, 22.2 mmol), several drops of glacial acetic acid and dichloroethane (20 mL) were successively added, and sodium triacetoxyborohydride (5.13 g, 24.2 mmol) was added in portions. The reaction was warmed to 50° C. and stirred for 4 hours. The reaction was cooled to room temperature, and 100 mL of dichloromethane was added. The organic phase was washed successively with distilled water (100 mL×2), saturated sodium bicarbonate solution (50 mL) and saturated brine (50 mL) and concentrated under reduced pressure to obtain a crude product as an oil. The crude product was further purified by column chromatography (eluent: EA/PE=1/1) to obtain compound 117B (1.8 g, 31.6%).

LC-MS (ESI): m/z=227.1[M−55]⁺.

Step 2:

### tert-butyl 4-((2-methyl-2H-tetrazol-5-yl)(phenyl)amino)piperidine-1-carboxylate (117C)

Compound 117B (0.9 g, 3.2 mmol), potassium tert-butoxide (0.55 g, 4.8 mmol), Pd₂(dba)₃ (0.44 g, 0.48 mmol), XantPhos (0.55 g, 0.96 mmol), bromobenzene (2.5 g, 16 mmol) and anhydrous toluene (20 mL) were successively added to a single-necked flask, and the reaction was stirred under nitrogen protection at 100° C. overnight. After TLC showed that the reaction was completed, the reaction solution was filtered through celite and concentrated under reduced pressure, and the residue was separated and purified by silica gel column chromatography (eluent: EA/PE=1/4) to obtain compound 117C (0.85 g, 74.4%).

LC-MS (ESI): m/z=303.3[M−55]⁺.

Step 3:

### N-(2-methyl-2H-tetrazol-5-yl)-N-phenylpiperidin-4-amine (117D)

Compound 117C (0.36 g, 1 mmol), DCM (15 mL) and trifluoroacetic acid (5 mL) were added to a single-necked flask, and the reaction was stirred at room temperature for 2 hours. After LCMS showed that the reaction was completed, the reaction solution was concentrated under reduced pressure to obtain a crude compound of 117D (0.24 g, 93.2%) from the residue. The crude product can be directly used in the next reaction.

LC-MS (ESI): m/z=259.1[M+H]⁺.

Step 4:

### Methyl-4-(5-((1S,2S)-2-fluorocyclopropane-1-carboxamido)benzo[d]oxazol-2-yl)picolinate (117E)

DMF (50 mL), (1S,2S)-2-fluorocyclopropanecarboxylic acid (425 mg, 4.1 mmol), HATU (2.1 g, 5.58 mmol) and DIEA (1.44 g, 11.16 mmol) were successively added to compound 4a (1.0 g, 3.71 mmol), and the mixture was stirred at room temperature for 5 h. The reaction was quenched by adding water, extracted 3 times with ethyl acetate and washed twice with saturated brine. The organic phase was dried and concentrated, and the residue was separated and purified by silica gel column chromatography (eluent: EA/PE=1/2) to obtain 117E (1.1 g, 83.4%).

LC-MS (ESI): m/z=356.3 [M+H]⁺.

Step 5:

### 4-(5-((1S,2S)-2-fluorocyclopropane-1-carboxamido)benzo[d]oxazol-2-yl)picolinic Acid (117F)

At room temperature, compound 117E (1 g, 3.1 mmol) was dissolved in methanol (15 mL), and lithium hydroxide (700 mg) was dissolved in 20 mL of pure water. An aqueous solution of lithium hydroxide was added to the reaction solution; the mixture was stirred at 40° C. for 0.5 hours and then adjusted to pH 6-7 with 2 N hydrochloric acid; and the resulting solution was extracted with ethyl acetate (30 mL×3). The combined organic phase was dried over anhydrous sodium sulfate and concentrated under reduced pressure to obtain 117F (1.0 g, 94.6%).

LC-MS (ESI): m/z=342.1 [M+H]⁺.

Step 6:

### (1S,2S)-2-fluoro-N-(2-(2-(4-((2-methyl-2H-tetrazol-5-yl)(phenyl)amino)piperidine-1-carbonyl)pyridin-4-yl)benzo[d]oxazol-5-yl)cyclopropane-1-carboxamide (Compound 117)

Under nitrogen protection, compound 117F (40 mg, 0.12 mmol), DMF (10 mL), DIPEA (30 mg, 0.22 mmol), HATU (55 mg, 0.14 mmol) and compound 117D (40 mg, 0.13 mmol) were successively added to a single-necked flask, and the mixture was stirred at room temperature for 3 h. The reaction was quenched by adding saturated aqueous sodium bicarbonate solution (10 mL), extracted with dichloromethane (50 mL×2) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by high-performance liquid column chromatography to obtain compound 117 (15 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.78 min.

¹H NMR (400 MHz, CDCl₃) δ 8.73 (s, 1H), 8.24 (s, 1H), 8.06 (s, 1H), 7.96 (s, 2H), 7.46-7.34 (m, 5H), 7.20-7.18 (m, 2H), 4.88-4.72 (m, 2H), 4.50 (t, 1H), 4.13 (s, 3H), 3.29 (t, 1H), 2.98 (t, 1H), 2.19-2.16 (m, 1H), 1.98-1.85 (m, 3H), 1.77-1.70 (m, 2H), 1.26-1.23 (m, 2H).

LC-MS (ESI): m/z=582.3 [M+H]⁺.

### Example 118

### Ethyl-(1-(4-(5-((1S,2S)-2-fluorocyclopropane-1-carboxamido)benzo[d]oxazol-2-yl)picolinoyl)piperidin-4-yl)(phenyl)carbamate

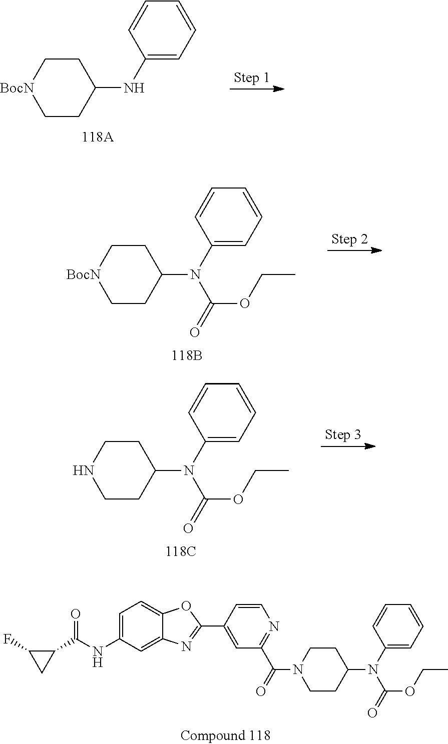 [SMILES: CC(C)(C)OC(=O)N1CCC(Nc2ccccc2)CC1 ; CCOC(=O)N(c1ccccc1)C1CCN(C(=O)OC(C)(C)C)CC1 ; CCOC(=O)N(c1ccccc1)C1CCNCC1 ; CCOC(=O)N(c1ccccc1)C1CCN(C(=O)c2cc(-c3nc4cc(NC(=O)[C@@H]5C[C@@H]5F)ccc4o3)ccn2)CC1]

Step 1:

### tert-butyl 4-((ethoxycarbonyl)(phenyl)amino)piperidine-1-carboxylate (118B)

To a single-necked flask, compound 118A (276 mg, 1 mmol), THE (10 mL), H₂O (3 mL) and sodium carbonate (160 mg, 1.5 mmol) were successively added, and ethyl chloroformate (140 mg, 1.3 mmol) was added dropwise under an ice bath. The reaction was warmed to room temperature and stirred for 2 hours. Dichloromethane (100 mL) was poured into the reaction solution, and the organic phase was washed successively with saturated sodium bicarbonate solution (50 mL) and saturated brine (50 mL) and concentrated under reduced pressure to obtain a crude product as an oil. The crude product was further purified by column chromatography (eluent: EA/PE=1/4) to obtain compound 118B (300 mg, 86.2%).

LC-MS (ESI): m/z=293.2[M−55]⁺.

Step 2:

### Ethyl phenyl(piperidin-4-yl)carbamate (118C)

Compound 118B (0.26 g, 1 mmol), DCM (15 mL) and trifluoroacetic acid (5 mL) were added to a single-necked flask, and the reaction was stirred at room temperature for 2 hours. After LCMS showed that the reaction was completed, the reaction solution was concentrated under reduced pressure to obtain a crude compound of 118C (0.18 g, 97.14%) from the residue. The crude product can be directly used in the next reaction.

LC-MS (ESI): m/z=249.3[M+H]⁺.

Step 3:

### Ethyl-(1-(4-(5-((1S,2S)-2-fluorocyclopropane-1-carboxamido)benzo[d]oxazol-2-yl)picolinoyl)piperidin-4-yl)(phenyl)carbamate

Under nitrogen protection, compound 117F (70 mg, 0.2 mmol), DMF (10 mL), DIPEA (50 mg, 0.38 mmol), HATU (91 mg, 0.24 mmol) and compound 118C (55 mg, 0.22 mmol) were successively added to a single-necked flask, and the mixture was stirred at room temperature for 3 h. The reaction was quenched by adding saturated aqueous sodium bicarbonate solution (10 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by high-performance liquid column chromatography to obtain compound 118 (20 mg, 17.06%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.13 min.

¹H NMR (400 MHz, CDCl₃) δ 8.72 (d, 1H), 8.25 (s, 1H), 8.15-8.04 (m, 1H), 7.98 (s, 1H), 7.79 (s, 1H), 7.57-7.44 (m, 2H), 7.42-7.31 (m, 3H), 7.11-7.03 (m, 2H), 5.00-4.64 (m, 2H), 4.50 (t, 1H), 4.12 (q, 2H), 4.02 (d, 1H), 3.22 (t, 1H), 2.90 (t, 1H), 2.04 (s, 1H), 1.96-1.78 (m, 3H), 1.54-1.41 (m, 2H), 1.34-1.21 (m, 2H), 1.15 (t, 2H).

LC-MS (ESI): m/z=572.3 [M+H]⁺.

### Example 119

### (R/S)-(4-((2-(difluoromethyl)-2H-tetrazol-5-yl)(phenyl)methyl)piperazin-1-yl)(4-(5-(thiazol-2-yl)benzo[d]oxazol-2-yl)pyridin-2-yl)methanone

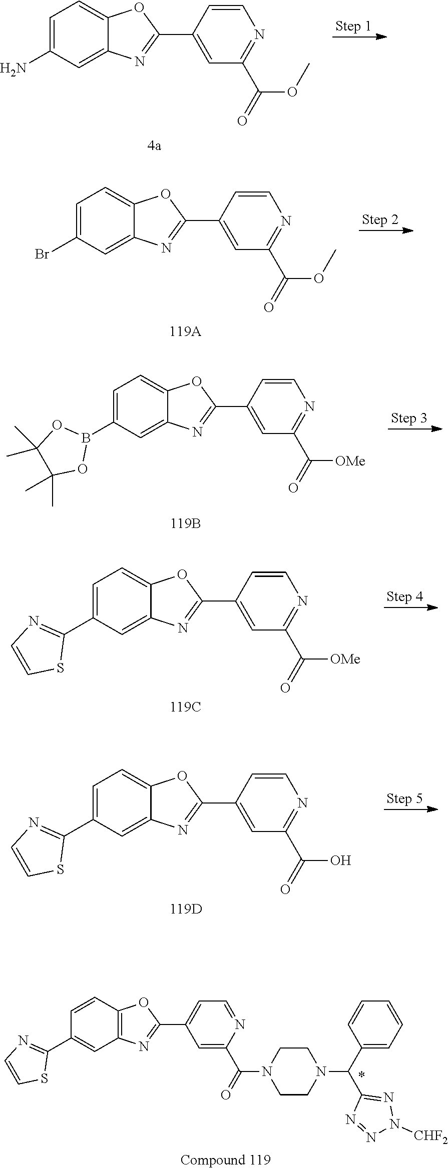

Step 1:

### Methyl 4-(5-bromobenzo[d]oxazol-2-yl)picolinate (119A)

Compound 4a (12.3 g, 45.7 mmol) was dissolved in acetonitrile (500 ml), and D-camphorsulfonic acid (12.7 g, 54.8 mmol), tert-butyl nitrite (5.7 g, 55.3 mmol), tetrabutylammonium bromide (29.4 g, 91.3 mmol) and CuBr₂ (0.1 g, 4.5 mmol) were added. After the addition was completed, the mixture was warmed to 60° C. and stirred overnight. The reaction solution was cooled to room temperature and filtered. The filter cake was washed twice with acetonitrile. The filtrate was combined and evaporated to dryness under reduced pressure. The residue was purified by silica gel column chromatography (eluent: PE/EA=4/1) to obtain compound 119A (6.0 g, 39.5%).

LC-MS (ESI): m/z=335.0[M+H+2]⁺.

Step 2:

### Methyl 4-(5-(4,4,5,5-tetramethyl-1,3,2-dioxaborolan-2-yl)benzo[d]oxazol-2-yl)picolinate (119B)

Compound 119A (8.0 g, 24.0 mmol) and bis(pinacolato)diboron (11.7 g, 48.0 mmol) were dissolved in 1,4-dioxane (100 ml), and potassium acetate (6.8 g, 69.3 mmol) was added. After the addition was completed, the mixture was subjected to nitrogen replacement 3 times, and then Pd(dppf)Cl₂.CH₂Cl₂ (1.0 g, 1.2 mmol) was added. The resulting mixture was then subjected to replacement with nitrogen 3 times, warmed to 100° C. and stirred for 4 hours. The reaction solution was cooled to room temperature and filtered. The filtrate was evaporated to dryness under reduced pressure. The residue was purified by silica gel column chromatography (eluent: PE/EA=5/1) to obtain 119B (8.0 g, 87.9%).

LC-MS (ESI): m/z=381.2[M+H]⁺.

### Step 3: methyl 4-(5-(thiazol-2-yl)benzo[d]oxazol-2-yl)picolinate (119C)

Compound 119B (500 mg, 1.3 mmol) and 2-bromothiazole (431 mg, 2.6 mmol) were placed in a microwave tube, and DMF (6.0 ml) and K₂CO₃ (363 mg, 2.6 mmol) were added. After the addition was completed, Pd(dppf)Cl₂.CH₂Cl₂ (107 mg, 0.13 mmol) was added under nitrogen protection, and then the mixture was reacted at 80° C. microwave for 1 hour. The reaction solution was evaporated to dryness under reduced pressure. The residue was purified by silica gel column chromatography (eluent: PE/EA=4/1) to obtain 119C (160 mg, 36.1%).

Step 4:

### 4-(5-(thiazol-2-yl)benzo[d]oxazol-2-yl)picolinic acid (119D)

Compound 119C (160 mg, 0.47 mmol) was dissolved in methanol (5.0 ml) and water (5.0 ml), and NaOH (57 mg, 1.4 mmol) was added. After the addition was completed, the mixture was stirred at room temperature overnight. The reaction solution was distilled under reduced pressure to remove methanol. The aqueous phase was adjusted to pH value=5 with 1 N dilute hydrochloric acid under ice bath cooling, stirred for 5 minutes and then filtered. The filter cake was dried to obtain 119D (140 mg, 91.5%).

LC-MS (ESI): m/z=324.1[M+H]⁺.

Step 5:

### (R/S)-(4-((2-(difluoromethyl)-2H-tetrazol-5-yl)(phenyl)methyl)piperazin-1-yl)(4-(5-(thiazol-2-yl)benzo[d]oxazol-2-yl)pyridin-2-yl)methanone (Compound 119)

Compound 119D (140 mg, 0.43 mmol) and intermediate 7 (128 mg, 0.43 mmol) were dissolved in DMF (5.0 ml), and DIPEA (168 mg, 1.3 mmol) and HATU (247 mg, 0.65 mmol) were added. After the addition was completed, the mixture was stirred at room temperature for 2 hours. Water (20 ml) was added to the reaction solution, and the mixture was extracted 3 times with EA. The organic phase was combined, washed twice with saturated aqueous NaCl solution, dried over anhydrous sodium sulfate and filtered. The filtrate was evaporated to dryness under reduced pressure. The residue was purified by preparative HPLC to obtain compound 119 (45 mg, 17.4%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.18 min.

LC-MS (ESI): m/z=600.3[M+H]⁺.

¹H NMR (400 MHz, DMSO-d₆) δ 8.82 (d, 1H), 8.64 (d, 1H), 8.42 (d, 1H), 8.21 (s, 1H), 8.19-8.18 (m, 1H), 8.15-8.14 (m, 1H), 7.99-7.97 (m, 2H), 7.81 (d, 1H), 7.54 (d, 2H), 7.44-7.31 (m, 3H), 5.48 (s, 1H), 3.76 (s, 2H), 3.57 (s, 2H), 2.71-2.58 (m, 4H).

### Examples 120 and 121

### (1S,2S)-2-fluoro-N-(2-(2-(4-((R/S)-(5-methyl-2H-tetrazol-2-yl)(phenyl)methyl)piperidine-1-carbonyl)pyridin-4-yl)benzo[d]oxazol-5-yl)cyclopropanecarboxamide and

### (1S,2S)-2-fluoro-N-(2-(2-(4-((S/R)-(5-methyl-2H-tetrazol-2-yl)(phenyl)methyl)piperidine-1-carbonyl)pyridin-4-yl)benzo[d]oxazol-5-yl)cyclopropanecarboxamide

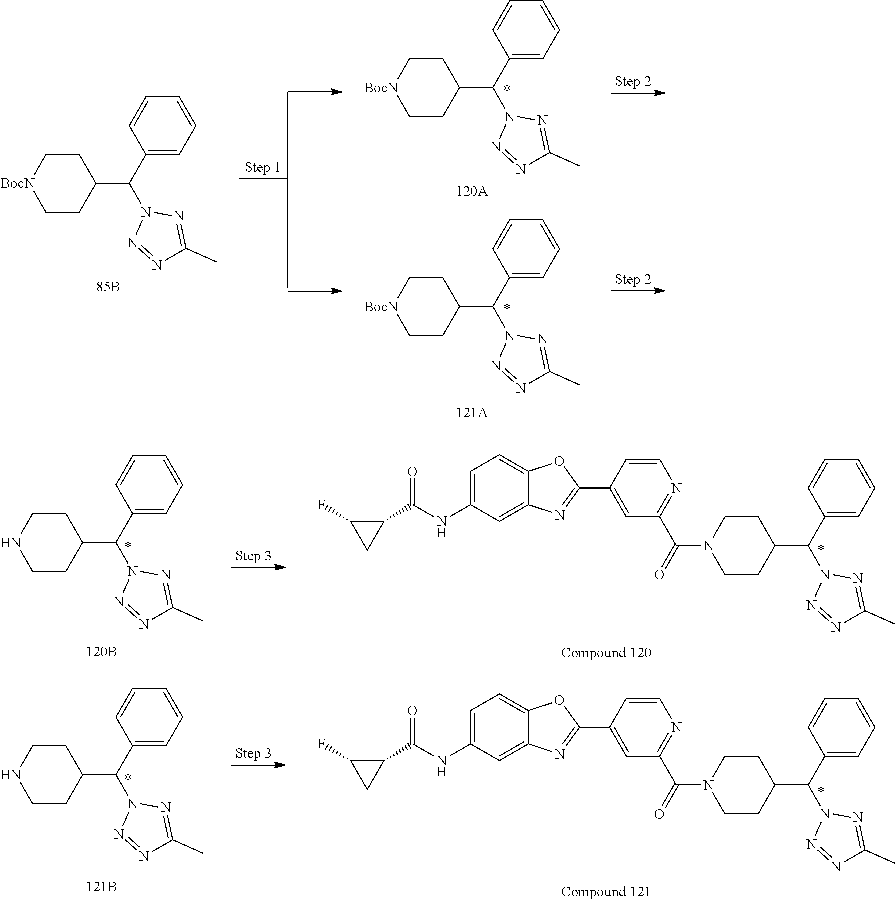

Step 1:

### (R)-tert-butyl 4-((5-methyl-2H-tetrazol-2-yl)(phenyl)methyl)piperidine-1-carboxylate and

### (S)-tert-butyl 4-((5-methyl-2H-tetrazol-2-yl)(phenyl)methyl)piperidine-1-carboxylate

The chiral resolution method of compound 85B was as follows: instrument: MG II preparative SFC (SFC-1); column type: ChiralCel OJ, 250×30 mm I.D., 5 μm; mobile phase: A: CO₂, B: ethanol; gradient: B 15%; flow rate: 60 mL/min; back pressure: 100 bar; column temperature: 38° C.; column length: 220 nm; time period: about 5 min; sample preparation: 3.0 g of compound 85B was dissolved in a mixed solvent (25 ml) of dichloromethane and methanol; sample injection: 2 ml each time.

Two isomers 120A (tR=1.78 min) and 121A (tR=2.60 min) were obtained.

Step 2:

### (R)-4-((5-methyl-2H-tetrazol-2-yl)(phenyl)methyl)piperidine

and

### (S)-4-((5-methyl-2H-tetrazol-2-yl)(phenyl)methyl)piperidine

120A (250 mg, 0.70 mmol) was dissolved in dichloromethane (10 mL) at room temperature; trifluoroacetic acid (2.5 mL) was added dropwise; and the mixture was stirred for another 2 hours; The reaction solution was subjected to rotary evaporation, and the residue was dissolved in dichloromethane (20 mL), neutralized to a basic pH with saturated NaHCO₃, washed with water (10 mL×2) and saturated sodium chloride solution (10 mL×1), dried over anhydrous sodium sulfate, filtered and concentrated to obtain 120B (145 mg, yield: 80%).

LC-MS (ESI): m/z=258.3[M+H]⁺.

Compound 121A was used as a raw material to obtain compound 121B according to the synthetic method of 120B.

LC-MS (ESI): m/z=258.3[M+H]⁺.

Step 3:

117F was used as a raw material to obtain the corresponding compound 120 and compound 121 by condensing with compound 120B and 121B according to the synthetic method of compound 85.

LC-MS (ESI): m/z=581.3[M+H]⁺.

Compound 120: ¹H NMR (400 MHz, CDCl₃) δ 8.74-8.71 (m, 1H), 8.31 (s, 1H), 8.12-8.10 (m, 1H), 8.01 (s, 1H), 7.90-7.87 (m, 1H), 7.56-7.51 (m, 4H), 7.41-7.31 (m, 3H), 5.55-5.52 (m, 1H), 4.93-4.75 (m, 2H), 3.91-3.88 (m, 1H), 3.20-3.11 (m, 1H), 2.91-2.80 (m, 2H), 2.56-2.50 (m, 3H), 1.94-1.84 (m, 2H), 1.61-1.23 (m, 5H).

Compound 121: ¹H NMR (400 MHz, CDCl₃) δ 8.75-8.73 (m, 1H), 8.34 (s, 1H), 8.16-8.13 (m, 1H), 8.04 (s, 1H), 7.88-7.85 (m, 1H), 7.56-7.51 (m, 4H), 7.41-7.34 (m, 3H), 5.56-5.53 (m, 1H), 4.94-4.76 (m, 2H), 3.87-3.84 (m, 1H), 3.20-3.16 (m, 1H), 2.91-2.80 (m, 2H), 2.56-2.50 (m, 3H), 1.94-1.84 (m, 2H), 1.61-1.23 (m, 5H).

### Examples 122 and 123

### (1S,2S)—N-(2-(2-(4-((R/S)-(2-(difluoromethyl)-2H-tetrazol-5-yl)(thiophen-3-yl)methyl)piperazine-1-carbonyl)pyridin-4-yl)benzo[d]oxazol-5-yl)-2-fluorocyclopropane-1-carboxamide and

### (1S,2S)—N-(2-(2-(4-((S/R)-(2-(difluoromethyl)-2H-tetrazol-5-yl)(thiophen-3-yl)methyl)piperazine-1-carbonyl)pyridin-4-yl)benzo[d]oxazol-5-yl)-2-fluorocyclopropane-1-carboxamide

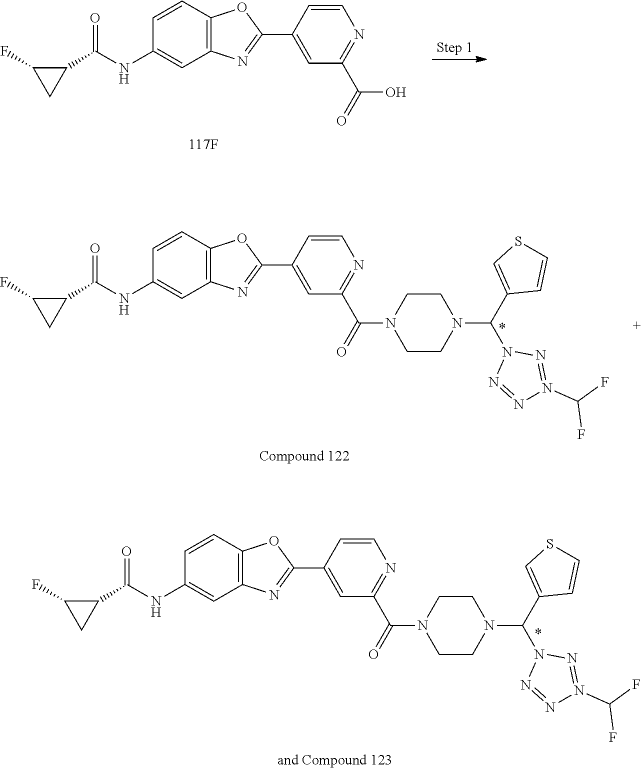

Step 1:

### (1S,2S)—N-(2-(2-(4-((R)-(2-(difluoromethyl)-2H-tetrazol-5-yl)(thiophen-3-yl)methyl)piperazine-1-carbonyl)pyridin-4-yl)benzo[d]oxazol-5-yl)-2-fluorocyclopropane-1-carboxamide and

### (1S,2S)—N-(2-(2-(4-((S)-(2-(difluoromethyl)-2H-tetrazol-5-yl)(thiophen-3-yl)methyl)piperazine-1-carbonyl)pyridin-4-yl)benzo[d]oxazol-5-yl)-2-fluorocyclopropane-1-carboxamide

DMF (20 mL), (1-((2-(difluoromethyl)-2H-tetrazol-5-yl) (thiophen-3-yl)methyl)piperazine hydrochloride (642 mg, 1.91 mmol), HATU (1088 mg, 2.9 mmol) and DIEA (739 mg, 5.73 mmol) were successively added to compound 117F (650 mg, 1.91 mmol), and the mixture was stirred at room temperature for 5 h. The reaction was quenched by adding water, extracted 3 times with ethyl acetate and washed twice with saturated brine. The organic phase was dried and concentrated to obtain a crude product compound, which was separated and purified by silica gel column chromatography and then subjected to chiral resolution to obtain compound 122 (30 mg, 5%) and compound 123 (25 mg, 4%) (compounds in single configurations). The resolution conditions of compounds 122 and 123 were as follows:

instrument: MG II preparative SFC (SFC-14); column type: ChiralPak AD, 250×30 mm I.D., 10 μm; mobile phase: A: CO₂, B: isopropanol (0.1% ammonia water); gradient: B 50%; flow rate: 80 mL/min; back pressure: 100 bar; column temperature: 38° C.; column length: 220 nm; time period: about 20 min; sample preparation: 180 mg of the compound was dissolved in a mixed solvent (15 ml) of dichloromethane and methanol; sample injection: 3.5 ml each time; retention time: 12.860 min for compound 122 and 19.632 min for compound 123.

### Compound 122

LC-MS (ESI): m/z=624.2 [M+H]⁺.

¹H NMR (400 MHz, DMSO-d₆) δ 10.48 (s, 1H), 8.80 (d, 1H), 8.42-8.72 (m, 1H), 8.24 (d, 1H), 8.15-8.21 (m, 2H), 7.79 (d, 1H), 7.50-7.65 (m, 3H), 7.24 (d, 1H), 5.62 (s, 1H), 4.85-5.05 (m, 1H), 3.73 (s, 2H), 3.52 (S, 2H), 2.50-2.80 (m, 4H), 2.05-2.10 (m, 1H), 1.55-1.75 (m, 1H), 1.10-1.25 (m, 1H).

### Compound 123

LC-MS (ESI): m/z=624.2 [M+H]⁺.

¹H NMR (400 MHz, DMSO-d₆) δ 10.48 (s, 1H), 8.80 (d, 1H), 8.42-8.72 (m, 1H), 8.24 (d, 1H), 8.15-8.21 (m, 2H), 7.79 (d, 1H), 7.50-7.65 (m, 3H), 7.24 (d, 1H), 5.62 (s, 1H), 4.85-5.05 (m, 1H), 3.73 (s, 2H), 3.52 (S, 2H), 2.51-2.81 (m, 4H), 2.05-2.10 (m, 1H), 1.55-1.75 (m, 1H), 1.10-1.25 (m, 1H).

### Example 124

### (R/S)—N-(2-(2-(4-((2-(difluoromethyl)-2H-tetrazol-5-yl)(phenyl)methyl)piperazine-1-carbonyl)pyridin-4-yl)-3-oxoisoindolin-5-yl)acetamide

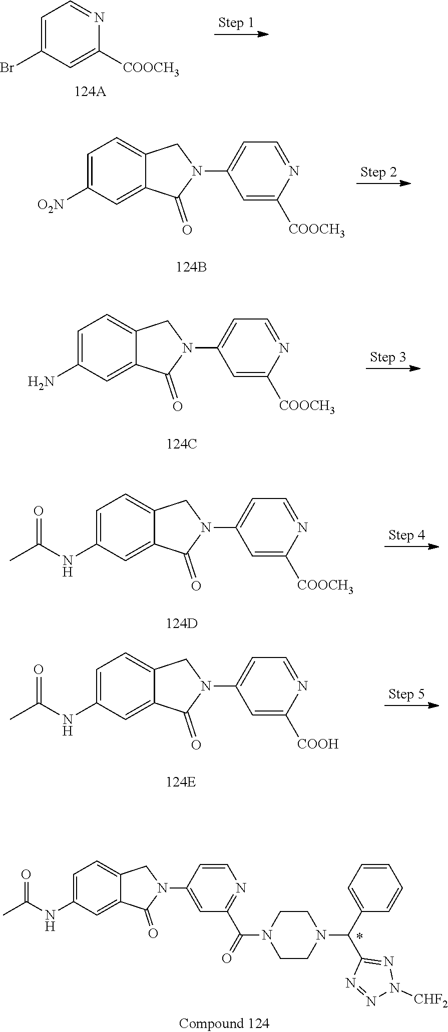 [SMILES: COC(=O)c1cc(Br)ccn1 ; COC(=O)c1cc(N2Cc3ccc([N+](=O)[O-])cc3C2=O)ccn1 ; COC(=O)c1cc(N2Cc3ccc(N)cc3C2=O)ccn1 ; COC(=O)c1cc(N2Cc3ccc(NC(C)=O)cc3C2=O)ccn1 ; CC(=O)Nc1ccc2c(c1)C(=O)N(c1ccnc(C(=O)O)c1)C2 ; CC(=O)Nc1ccc2c(c1)C(=O)N(c1ccnc(C(=O)N3CCN(C(c4ccccc4)c4nnn(C(F)F)n4)CC3)c1)C2]

Step 1:

### Methyl 4-(6-nitro-1-oxoisoindolin-2-yl)picolinate (124B)

Compound 124A (500 mg, 2.31 mmol) was added to dioxane (20 mL), and then 6-nitroisoindolin-1-one (412 mg, 2.31 mmol), cesium carbonate (1130 mg, 3.47 mmol), Pd₂(dba)₃ (92 mg, 0.1 mmol) and Xphos (95 mg, 0.2 mmol) were added, and under nitrogen protection, the mixture was warmed to 100° C. and stirred for 6 h. The reaction was cooled to room temperature, and the filtrate was concentrated and then separated and purified by silica gel column chromatography (PE:EA=1:1) to obtain 124B (300 mg, 41%).

LC-MS (ESI): m/z=314.1[M+H]⁺.

Step 2:

### Methyl 4-(6-amino-1-oxoisoindolin-2-yl)picolinate (124C)

Compound 124B (0.30 g, 0.96 mmol) was added to methanol (50 mL); then palladium on carbon (0.03 g) was added; and the mixture was stirred at 25° C. for 10 hours under the protection of hydrogen replacement. The reaction was filtered, and the filtrate was concentrated to obtain 124C as a light yellow solid (0.20 g, 74%).

LC-MS (ESI): m/z=284.1 [M+H]⁺.

Step 3:

### Methyl 4-(6-acetamido-1-oxoisoindolin-2-yl)picolinate (124D)

Compound 124C (300 mg, 1.06 mmol) was added to dichloromethane (10 mL), and then triethylamine (321 mg, 3.18 mmol) was added. The mixture was cooled to 0-5° C. in an ice bath, and acetyl chloride (832 mg, 10.6 mmol) was added. After the addition, the mixture was reacted for 2 hours; water (20 mL) was added; and the resulting solution was extracted twice with dichloromethane (20 mL×2). The organic phase was combined, dried and concentrated to obtain a crude product of compound 124D (500 mg, 100%).

LC-MS (ESI): m/z=326.1 [M+H]⁺.

Step 4:

### 4-(6-acetamido-1-oxoisoindolin-2-yl)picolinic Acid (124E)

Anhydrous methanol (15 mL) and NaOH (0.25 g, 6.15 mmol, 2 mL) aqueous solution were successively added to compound 124D (0.5 g, 1.54 mmol). The mixture was stirred at room temperature overnight, concentrated under reduced pressure to remove most of methanol, adjusted to pH=2-3 by dropwise adding 1 N hydrochloric acid and filtered. The filter cake was washed with water (3 mL) and dried to obtain compound 124E (150 mg, 33%).

LC-MS (ESI): m/z=312.1 [M−H]⁻.

Step 5:

### (R/S)—N-(2-(2-(4-((2-(difluoromethyl)-2H-tetrazol-5-yl)(phenyl)methyl)piperazine-1-carbonyl)pyridin-4-yl)-3-oxoisoindolin-5-yl)acetamide

At room temperature, DMF (8 mL), compound intermediate 7 (76 mg, 0.26 mmol), HATU (148 mg, 0.39 mmol) and DIPEA (101 mg, 0.78 mmol) were successively added to compound 124E (80 mg, 0.26 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 124 (18 mg, 12%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 14.15 min.

LC-MS (ESI): m/z=588.3 [M+H]⁺.

¹H NMR (400 MHz, DMSO-d₆) δ 10.22 (s, 1H), 8.55 (t, 1H), 8.52 (d, 1H), 8.18 (d, 1H), 8.06 (d, 1H), 7.90-7.92 (m, 1H), 7.76-7.78 (m, 1H), 7.59-7.61 (m, 1H), 7.48-7.50 (m, 2H), 7.37-7.41 (m, 2H), 7.31-7.35 (m, 1H), 5.31 (s, 1H), 4.99 (s, 2H), 3.68-3.70 (m, 2H), 3.44-3.45 (m, 2H), 2.56-2.59 (m, 1H), 2.43-2.49 (m, 2H), 2.32-2.35 (m, 1H), 2.09 (s, 3H).

### Example 125

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-6-yl)-1-fluorocyclopropane-1-carboxamide

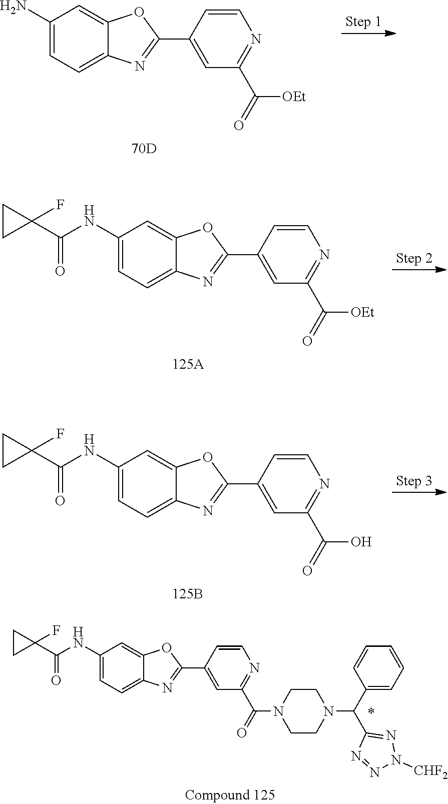 [SMILES: CCOC(=O)c1cc(-c2nc3ccc(N)cc3o2)ccn1 ; CCOC(=O)c1cc(-c2nc3ccc(NC(=O)C4(F)CC4)cc3o2)ccn1 ; O=C(O)c1cc(-c2nc3ccc(NC(=O)C4(F)CC4)cc3o2)ccn1 ; O=C(c1cc(-c2nc3ccc(NC(=O)C4(F)CC4)cc3o2)ccn1)N1CCN(C(c2ccccc2)c2nnn(C(F)F)n2)CC1]

Step 1:

### Ethyl 4-(6-(1-fluorocyclopropane-1-carboxamido)benzo[d]oxazol-2-yl)picolinate (125A)

At room temperature, DMF (8 mL), 1-fluorocyclopropanecarboxylic acid (75 mg, 0.72 mmol), HATU (374 mg, 0.98 mmol) and DIPEA (0.34 g, 2.62 mmol) were successively added to compound 70D (187 mg, 0.66 mmol). The reaction was stirred for 1 hour. The reaction solution was poured into water (30 mL) and extracted with ethyl acetate (30 mL×4). The organic phase was concentrated, and the residue was separated and purified by silica gel column chromatography (PE:EA=1:4) to obtain compound 125A (200 mg, 82%).

LC-MS (ESI): m/z=370.1 [M+H]⁺.

Step 2:

### 4-(6-(1-fluorocyclopropane-1-carboxamido)benzo[d]oxazol-2-yl)picolinic Acid (125B)

Anhydrous methanol (10 mL) and NaOH (108 mg, 2.70 mmol, 1 mL) aqueous solution were successively added to compound 125A (200 mg, 0.54 mmol), and the reaction was stirred at 25° C. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (3 mL) was added, and the mixture was stirred and then filtered. The filter cake was dried to obtain intermediate 125B (180 mg, 98%).

LC-MS (ESI): m/z=342.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-6-yl)-1-fluorocyclopropane-1-carboxamide (Compound 125)

At room temperature, DMF (8 mL), intermediate 7 (85 mg, 0.29 mmol), HATU (165 mg, 0.44 mmol) and DIPEA (112 mg, 0.87 mmol) were successively added to compound 125B (100 mg, 0.29 mmol). The reaction was stirred for 1 hour. TLC showed that the reaction of the raw materials was completed. The reaction solution was directly poured into water (30 mL) and extracted with ethyl acetate (30 mL×3). The organic phase was concentrated, and the residue was separated and purified by HPLC to obtain target compound 125 (40 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.33 min.

LC-MS (ESI): m/z=618.3 [M+H]⁺.

¹H NMR (400 MHz, DMSO-d₆) δ 10.58 (s, 1H), 8.79-8.81 (m, 1H), 8.55 (t, 1H), 8.31-8.32 (m, 1H), 8.15-8.16 (m, 1H), 8.12-8.14 (m, 1H), 7.78-7.86 (m, 2H), 7.48-7.50 (m, 2H), 7.35-7.41 (m, 2H), 7.31-7.33 (m, 1H), 5.32 (s, 1H), 3.71-3.72 (m, 2H), 3.49-3.50 (m, 2H), 2.58-2.61 (m, 1H), 2.49-2.52 (m, 2H), 2.34-2.36 (m, 1H), 1.46-1.51 (m, 1H), 1.42-1.45 (m, 1H), 1.34-1.39 (m, 2H).

### Example 126

### (R/S)-(4-((2-(difluoromethyl)-2H-tetrazol-5-yl)(phenyl)methyl)piperazin-1-yl)(4-(5-(1-methyl-1H-imidazol-5-yl)benzo[d]oxazol-2-yl)pyridin-2-yl)methanone

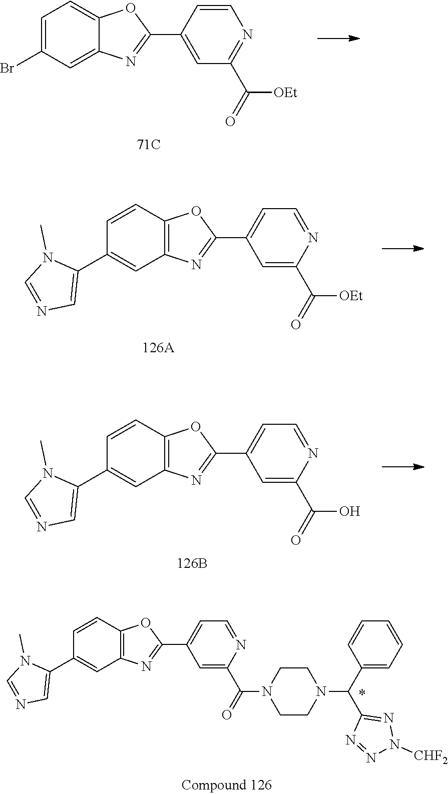 [SMILES: CCOC(=O)c1cc(-c2nc3cc(Br)ccc3o2)ccn1 ; CCOC(=O)c1cc(-c2nc3cc(-c4cncn4C)ccc3o2)ccn1 ; Cn1cncc1-c1ccc2oc(-c3ccnc(C(=O)O)c3)nc2c1 ; Cn1cncc1-c1ccc2oc(-c3ccnc(C(=O)N4CCN(C(c5ccccc5)c5nnn(C(F)F)n5)CC4)c3)nc2c1]

Step 1:

### Ethyl 4-(5-(1-methyl-1H-imidazol-5-yl)benzo[d]oxazol-2-yl)picolinate (126A)

Compound 71C (260 mg, 0.75 mmol) was added to a mixed solvent of dioxane (10 mL) and water (1 mL), and then 1-methyl-5-(4,4,5,5-tetramethyl-1,3,2-dioxin-2-yl)-1H imidazole (312 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 126A (220 mg, 84%).

LC-MS (ESI): m/z=349.1[M+H]⁺.

Step 2:

### 4-(5-(1-methyl-1H-imidazol-5-yl)benzo[d]oxazol-2-yl)picolinic Acid (126B)

Anhydrous methanol (10 mL) and NaOH (126 mg, 3.15 mmol, 2 mL) aqueous solution were successively added to compound 126A (220 mg, 0.63 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent and then obtain a crude product of intermediate 126B (300 mg).

LC-MS (ESI): m/z=321.1 [M+H]⁺.

Step 3:

### (R/S)-(4-((2-(difluoromethyl)-2H-tetrazol-5-yl)(phenyl)methyl)piperazin-1-yl)(4-(5-(1-methyl-1H-imidazol-5-yl)benzo[d]oxazol-2-yl)pyridin-2-yl)methanone (Compound 126)

At room temperature, DMF (8 mL), intermediate 7 (110 mg, 0.38 mmol), HATU (217 mg, 0.57 mmol) and DIPEA (147 mg, 1.14 mmol) were successively added to compound 126B (120 mg, 0.38 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 126 (30 mg, 13%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 14.56 min.

LC-MS (ESI): m/z=597.3 [M+H]⁺.

¹H NMR (400 MHz, DMSO-d₆) δ8.81-8.82 (m, 1H), 8.55 (t, 1H), 8.20-8.21 (m, 1H), 8.16-8.18 (m, 1H), 8.02 (d, 1H), 7.94 (d, 1H), 7.74 (s, 1H), 7.62-7.65 (m, 1H), 7.49-7.51 (m, 2H), 7.37-7.41 (m, 2H), 7.31-7.35 (m, 1H), 7.13 (d, 1H), 5.32 (s, 1H), 3.73 (s, 3H), 3.72-3.73 (m, 2H), 3.51-3.52 (m, 2H), 2.58-2.61 (m, 1H), 2.47-2.49 (m, 2H), 2.35-2.37 (m, 1H).

### Example 127

### (R/S)-(4-((2-(difluoromethyl)-2H-tetrazol-5-yl)(phenyl)methyl)piperazin-1-yl)(4-(5-(1-ethyl-1H-pyrazol-4-yl)benzo[d]oxazol-2-yl)pyridin-2-yl)methanone

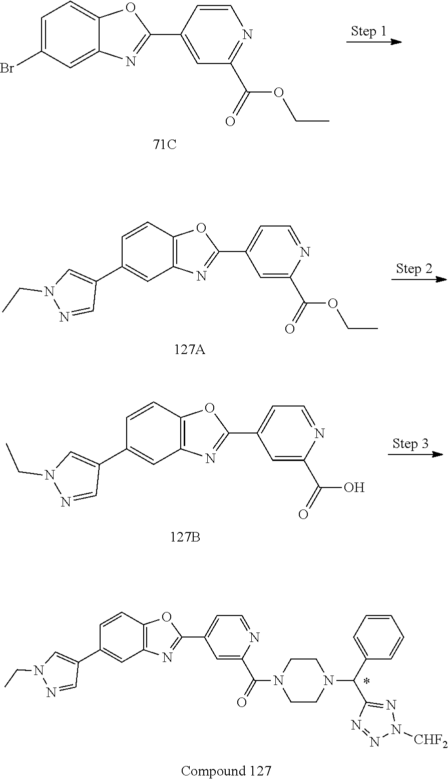

Step 1:

### Ethyl 4-(5-(1-ethyl-1H-pyrazol-4-yl)benzo[d]oxazol-2-yl)picolinate (127A)

Compound 71C (260 mg, 0.75 mmol) was added to a mixed solvent of dioxane (10 mL) and water (1 mL), and then 1-ethyl-4-(4,4,5,5-tetramethyl-1,3,2-dioxin-2-yl)-1H pyrazole (333 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 127A (180 mg, 66%).

LC-MS (ESI): m/z=363.1[M+H]⁺.

Step 2:

### 4-(5-(1-ethyl-1H-pyrazol-4-yl)benzo[d]oxazol-2-yl)picolinic Acid (127B)

Anhydrous methanol (10 mL) and NaOH (100 mg, 2.49 mmol, 2 mL) aqueous solution were successively added to compound 127A (180 mg, 0.50 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 127B (150 mg, 90%).

LC-MS (ESI): m/z=335.1 [M+H]⁺.

Step 3:

### (R/S)-(4-((2-(difluoromethyl)-2H-tetrazol-5-yl)(phenyl)methyl)piperazin-1-yl)(4-(5-(1-ethyl-1H-pyrazol-4-yl)benzo[d]oxazol-2-yl)pyridin-2-yl)methanone (Compound 127)

At room temperature, DMF (8 mL), intermediate 7 (132 mg, 0.45 mmol), HATU (257 mg, 0.68 mmol) and DIPEA (174 mg, 1.35 mmol) were successively added to compound 127B (150 mg, 0.45 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 127 (35 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 15.16 min.

LC-MS (ESI): m/z=611.3 [M+H]⁺.

¹H NMR (400 MHz, DMSO-d₆) δ8.81-8.82 (m, 1H), 8.55 (t, 1H), 8.29 (s, 1H), 8.18-8.19 (m, 1H), 8.14-8.16 (m, 1H), 8.08 (d, 1H), 7.97-7.98 (m, 1H), 7.83 (d, 1H), 7.73-7.75 (m, 1H), 7.49-7.51 (m, 2H), 7.37-7.41 (m, 2H), 7.31-7.35 (m, 1H), 5.32 (s, 1H), 4.14-4.20 (q, 2H), 3.72-3.73 (m, 2H), 3.51-3.52 (m, 2H), 2.58-2.61 (m, 1H), 2.47-2.49 (m, 2H), 2.33-2.37 (m, 1H), 1.43 (t, 3H).

### Example 128

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-6-yl)deuterated Acetamide

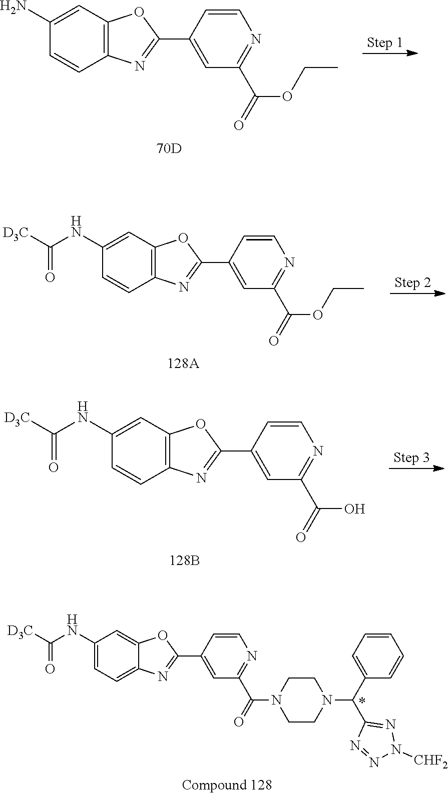 [SMILES: CCOC(=O)c1cc(-c2nc3ccc(N)cc3o2)ccn1 ; [2H]C([2H])([2H])C(=O)Nc1ccc2nc(-c3ccnc(C(=O)OCC)c3)oc2c1 ; [2H]C([2H])([2H])C(=O)Nc1ccc2nc(-c3ccnc(C(=O)O)c3)oc2c1 ; [2H]C([2H])([2H])C(=O)Nc1ccc2nc(-c3ccnc(C(=O)N4CCN(C(c5ccccc5)c5nnn(C(F)F)n5)CC4)c3)oc2c1]

Step 1:

### Ethyl 4-(6-deuteratedacetamidobenzo[d]oxazol-2-yl)picolinate (128A)

Deuterated acetic acid (200 mg, 3 mmol) and 70D (300 mg, 1 mmol) were dissolved in N,N-dimethylformamide (5 mL). HATU (570 mg, 1.5 mmol) and DIPEA (516 mg, 4 mmol) were added. After the addition, the mixture was stirred at room temperature for 1 hour. The reaction was quenched by adding water (30 mL) and directly filtered. The filtered solid was washed with water and dried under reduced pressure to obtain compound 128A (250 mg, 76%).

LC-MS (ESI): m/z=329.1 [M+H]⁺.

Step 2:

### 4-(6-deuteratedacetamidobenzo[d]oxazol-2-yl)picolinic Acid (128B)

Compound 128A (250 mg, 0.76 mmol) and sodium hydroxide (152 mg, 3.8 mmol) were added to a mixed system of methanol (5 mL) and water (2 ml), and the mixture was reacted at room temperature for 2 hours. The reaction solution was adjusted to pH=1-2 with dilute hydrochloric acid, and a small amount of acetone was added to the reaction solution. The mixture was filtered, and the filtered solid was washed with water to obtain compound 128B (150 mg, 66%).

LC-MS (ESI): m/z=301.1 [M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-6-yl) deuterated Acetamide (Compound 128)

Intermediate 7 (147 mg, 0.5 mmol) and 128B (150 mg, 0.5 mmol) were dissolved in N,N-dimethylformamide (5 mL). HATU (285 mg, 0.75 mmol) and DIPEA (194 mg, 1.5 mmol) were added. After the addition, the mixture was stirred for 1 hour at room temperature, and then the reaction was quenched by adding water (30 mL). The resulting solution was extracted twice with ethyl acetate (30 mL×3). The organic phase was combined, dried over anhydrous sodium sulfate and concentrated under reduced pressure, and then the residue was separated by column chromatography (eluent: MeOH/DCM=1/20) to obtain compound 128 (100 mg, 35%).

¹H NMR (400 MHz, MeOD) δ 8.76 (m, 1H), 8.32-8.00 (m, 4H), 7.72 (m, 1H), 7.54-7.52 (m, 2H), 7.43-7.28 (m, 4H), 5.19 (s, 1H), 3.86 (t, 2H), 3.58 (t, 2H), 2.76-2.66 (m, 1H), 2.64-2.50 (m, 2H), 2.48-2.38 (m, 1H).

LC-MS (ESI): m/z=577.2[M+H]⁺.

### Example 129

### (R/S)—N-(2-(6-(4-((2-(difluoromethyl)-2H-tetrazol-5-yl)(phenyl)methyl)piperazine-1-carbonyl)pyridin-2-yl)benzo[d]oxazol-5-yl)-1-fluorocyclopropane-1-carboxamide

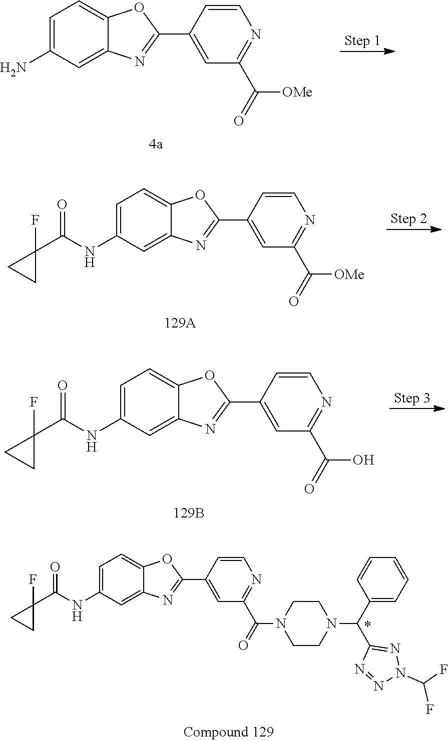 [SMILES: COC(=O)c1cc(-c2nc3cc(N)ccc3o2)ccn1 ; COC(=O)c1cc(-c2nc3cc(NC(=O)C4(F)CC4)ccc3o2)ccn1 ; O=C(O)c1cc(-c2nc3cc(NC(=O)C4(F)CC4)ccc3o2)ccn1 ; O=C(c1cc(-c2nc3cc(NC(=O)C4(F)CC4)ccc3o2)ccn1)N1CCN(C(c2ccccc2)c2nnn(C(F)F)n2)CC1]

Step 1:

### Methyl 4-(5-(1-fluorocyclopropanecarboxamido)benzo[d]oxazol-2-yl)picolinate (129A)

DCM (10 mL), 1-fluorocyclopropanecarboxylic acid (0.15 g, 1.5 mmol), HATU (0.56 g, 1.50 mmol) and DIPEA (0.48 g, 3.7 mmol) were successively added to compound 4a (0.35 g, 1.20 mmol), and the mixture was stirred at room temperature for 5 h. The reaction was quenched by adding water, extracted 3 times with ethyl acetate and washed twice with saturated brine. The organic phase was dried and concentrated, and the residue was separated and purified by silica gel column chromatography (PE/EA=5:1) to obtain 129A (0.4 g, 88.0%).

LC-MS (ESI): m/z=356.1 [M+H]⁺.

Step 2:

### 4-(5-(1-fluorocyclopropanecarboxamido)benzo[d]oxazol-2-yl)picolinic Acid (129B)

At room temperature, compound 129A (0.4 g, 1.1 mmol) was dissolved in methanol (10 mL), and lithium hydroxide (0.1 g) was dissolved in 2 mL of pure water. An aqueous solution of lithium hydroxide was added to the reaction solution; the mixture was stirred at 40° C. for 0.5 hours and then adjusted to pH 6-7 with 2 N hydrochloric acid; and the resulting solution was extracted with ethyl acetate (30 mL×3). The combined organic phase was dried over anhydrous sodium sulfate and concentrated under reduced pressure to obtain 129B (0.26 g, 70.1%).

LC-MS (ESI): m/z=342.1 [M+H]⁺.

Step 3:

### (R/S)—N-(2-(6-(4-((2-(difluoromethyl)-2H-tetrazol-5-yl)(phenyl)methyl)piperazine-1-carbonyl)pyridin-2-yl)benzo[d]oxazol-5-yl)-1-fluorocyclopropane-1-carboxamide

Intermediate 129B (0.26 g, 0.76 mmol) and DMF (10 ml) were added, and DIPEA (0.30 g, 2.3 mmol), HATU (0.35 g, 0.91 mmol) and intermediate 7 (0.27 g, 0.91 mmol) were added. After the addition, the mixture was reacted at room temperature for 2 h. The reaction solution was adjusted to neutral by dropwise adding saturated aqueous ammonium chloride solution, and 30 ml of saturated aqueous sodium chloride solution was added. The mixture was extracted with ethyl acetate (25 ml×3). The organic layer was combined, dried, filtered and concentrated, and the residue was separated by column chromatography (DCM/MeOH=50:1-10:1) to obtain product 129 (0.10 g, 21%).

LC-MS (ESI): m/z=618.2[M+1]⁺.

¹H NMR (400 MHz, CDCl₃) δ 8.73 (dd, 1H), 8.40 (d, 1H), 8.26 (d, 1H), 8.17-8.03 (m, 2H), 7.79-7.29 (m, 7H), 5.17 (s, 1H), 3.92 (s, 2H), 3.72 (s, 2H), 2.63 (dd, 4H), 1.59-1.34 (m, 4H).

### Example 130

### (R/S)-(4-((2-(difluoromethyl)-2H-tetrazol-5-yl)(phenyl)methyl)piperazin-1-yl)(4-(5-(1-methyl-1H-1,2,3-triazol-4-yl)benzo[d]oxazol-2-yl)pyridin-2-yl)methanone

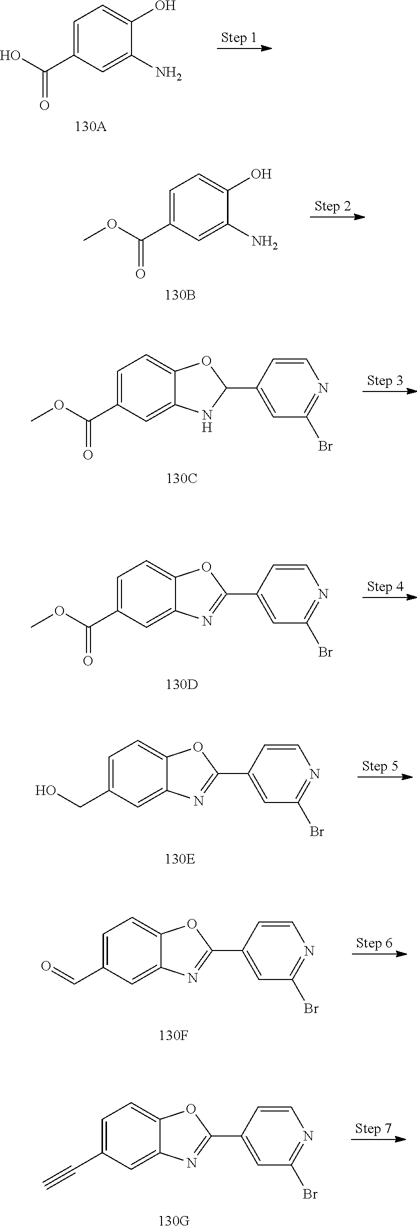

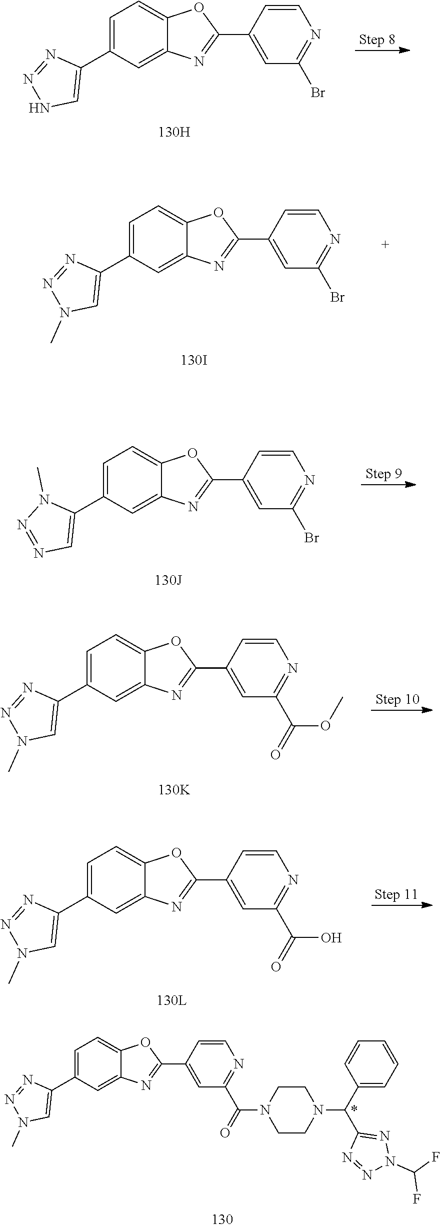 [SMILES: Brc1cc(-c2nc3cc(-c4c[nH]nn4)ccc3o2)ccn1 ; Cn1cc(-c2ccc3oc(-c4ccnc(Br)c4)nc3c2)nn1 ; Cn1nncc1-c1ccc2oc(-c3ccnc(Br)c3)nc2c1 ; COC(=O)c1cc(-c2nc3cc(-c4cn(C)nn4)ccc3o2)ccn1 ; Cn1cc(-c2ccc3oc(-c4ccnc(C(=O)O)c4)nc3c2)nn1 ; Cn1cc(-c2ccc3oc(-c4ccnc(C(=O)N5CCN(C(c6ccccc6)c6nnn(C(F)F)n6)CC5)c4)nc3c2)nn1]

Step 1:

### Methyl 3-amino-4-hydroxybenzoate (130B)

Compound 130A (15.3 g, 100 mmol) was dissolved in anhydrous methanol (150 mL); thionyl chloride (11.9 g, 100 mmol) was added dropwise under an ice-water bath condition; and after the dropwise addition was completed, the mixture was refluxed at 65° C. for 2 h. The reaction was quenched by adding water, concentrated under reduced pressure to remove the organic solvent, adjusted to pH=7-8 with saturated aqueous sodium bicarbonate solution, extracted 3 times with ethyl acetate and washed once with saturated brine. The organic phase was dried and concentrated to obtain 130B (17 g, 100%).

LC-MS (ESI): m/z=168.0 [M+H]⁺.

Step 2:

### Methyl 2-(2-bromopyridin-4-yl)-2,3-dihydrobenzo[d]oxazole-5-carboxylate (130C)

At room temperature, compound 130B (17.0 g, 100 mmol) was dissolved in methanol (200 mL), and 2-bromoisonicotinaldehyde (22.3 g, 120 mmol) was added. The mixture was stirred at 60° C. for 6 h and after TLC showed that the reaction was completed, the mixture was concentrated under reduced pressure to obtain 130C (39 g, 100%).

Step 3:

### Methyl 2-(2-bromopyridin-4-yl)benzo[d]oxazole-5-carboxylate (130D)

Compound 130C (39.0 g, 100 mmol) was dissolved in DCM (200 mL); 58% activated manganese dioxide (87 g, 1 mol) was added; and the mixture was stirred at room temperature for 12 h. The reaction solution was filtered, and the filter cake was washed with methanol. The filtrate was dried and concentrated to obtain a crude product compound, which was separated and purified by silica gel column chromatography (EA:PE=20%-40%) to obtain compound 130D (15 g, 45%).

LC-MS (ESI): m/z=333.1 [M+H]⁺.

Step 4:

### (2-(2-bromopyridin-4-yl)benzo[d]oxazol-5-yl)methanol (130E)

Tetrahydrofuran (100 mL) and lithium aluminum tetrahydride (1.1 g, 30.0 mmol) (under an ice-water bath) were successively added to compound 130D (10.0 g, 30.0 mmol), and the mixture was stirred at room temperature for 0.5 h. The reaction was quenched by adding water (1.1 mL). Then 15% sodium hydroxide aqueous solution (1.1 mL) was added, and then water (3.3 mL) was added. The mixture was filtered, and then the filtrate was dried and concentrated to obtain compound 130E (8.0 g, 88%).

LC-MS (ESI): m/z=305.1 [M+H]⁺.

Step 5:

### 2-(2-bromopyridin-4-yl)benzo[d]oxazole-5-carbaldehyde (130F)

Dichloromethane (100 mL) and Dess-Martin reagent (11.2 g, 26.3 mmol) were successively added to compound 130E (8.0 g, 26.3 mmol), and the mixture was stirred at room temperature for 1 h. The reaction solution was filtered. The filtrate was dried and concentrated to obtain a crude product compound, which was separated and purified by silica gel column chromatography to obtain compound 130F (7 g, 87%).

LC-MS (ESI): m/z=303.1 [M+H]⁺.

Step 6:

### 2-(2-bromopyridin-4-yl)-5-ethynylbenzo[d]oxazole (130G)

Methanol (100 mL), potassium carbonate (6.4 g, 46.2 mmol), dimethyl (1-diazo-2-oxopropyl)phosphonate (6.7 g, 34.7 mmol) were successively added to 130F (7.0 g, 23.1 mmol), and the mixture was stirred at room temperature for 12 h. The reaction was quenched by adding water, concentrated under reduced pressure to remove the organic solvent, extracted 3 times with dichloromethane and washed once with saturated brine. The organic phase was dried and concentrated to obtain a crude product compound, which was separated and purified by silica gel column chromatography (EA:PE=10%-20%) to obtain compound 130G (2.7 g, 39%).

LC-MS (ESI): m/z=299.1 [M+H]⁺.

Step 7:

### 2-(2-bromopyridin-4-yl)-5-(1H-1,2,3-triazol-4-yl)benzo[d]oxazole (130H)

Azidotrimethylsilane (50 mL) was added to compound 130G (2.5 g, 8.3 mmol), and the mixture was reacted in a microwave reactor at 150° C. for 1 h and filtered to obtain the filter cake, which was compound 130H (2.7 g, 39%).

LC-MS (ESI): m/z=342.2 [M+H]⁺.

Step 8:

### 2-(2-bromopyridin-4-yl)-5-(1-methyl-1H-1,2,3-triazol-4-yl)benzo[d]oxazole (130I)

### 2-(2-bromopyridin-4-yl)-5-(1-methyl-1H-1,2,3-triazol-5-yl)benzo[d]oxazole (130J)

DMF (50 mL), potassium carbonate (800 mg, 5.8 mmol) and iodomethane (823 mg, 5.8 mmol) were added to compound 130H (2.0 g, 5.8 mmol), and the mixture was reacted at room temperature overnight, quenched by adding water, extracted 3 times with ethyl acetate, washed 3 times with water and washed once with saturated brine. The organic phase was dried and concentrated to obtain a crude product compound, which was separated and purified by silica gel column chromatography (PE/EA=3/5) (developing solvent (PE/EA=3/5)) to obtain compounds 1301 (1.5 g, 70%, Rf value: 0.4) and 130J (400 mg, 20%, Rf value: 0.2).

LC-MS (ESI): m/z=356.2 [M+H]⁺. (130I)

LC-MS (ESI): m/z=356.2 [M+H]⁺. (130J)

Step 9:

### Methyl 4-(5-(1-methyl-1H-1,2,3-triazol-4-yl)benzo[d]oxazol-2-yl)picolinate (130K)

Compound 130I (500 mg, 1.4 mmol), [1,1′-bis(diphenylphosphino)ferrocene]palladium dichloride dichloromethane complex (100 mg) and triethylamine (424 mg, 4.2 mmol) were added to a mixed system of methanol (20 mL) and dichloromethane (20 ml), and the mixture was reacted at 110° C. under carbon monoxide (20 MPa) in an autoclave for 3 hours. The reaction solution was cooled to room temperature and filtered. The filtrate was concentrated under reduced pressure, and then the residue was subjected to column chromatography (EA:PE=20%-60%) to obtain compound 130K (350 mg, 74%).

LC-MS (ESI): m/z=336.3 [M+H]⁺.

Step 10:

### 4-(5-(1-methyl-1H-1,2,3-triazol-4-yl)benzo[d]oxazol-2-yl)picolinic Acid (130L)

Compound 130K (350 mg, 1.04 mmol) was added to a mixed system of methanol (20 mL), lithium hydroxide (350 mg) and water (20 ml), and the mixture was reacted at 45° C. for 1 hour. The reaction solution was adjusted to pH=5-6 with 2 M hydrochloric acid and filtered to obtain the filter cake, which was compound 130 L (200 mg, 59%).

LC-MS (ESI): m/z=322.1 [M+H]⁺.

Step 11:

### (R/S)-(4-((2-(difluoromethyl)-2H-tetrazol-5-yl)(phenyl)methyl)piperazin-1-yl)(4-(5-(1-methyl-1H-1,2,3-triazol-4-yl)benzo[d]oxazol-2-yl)pyridin-2-yl)methanone (Compound 130)

Intermediate 7 (183 mg, 0.62 mmol) and 130L (200 mg, 0.62 mmol) were dissolved in N,N-dimethylformamide (20 mL). HATU (353 mg, 0.93 mmol) and DIPEA (160 mg, 1.24 mmol) were added. After the addition, the mixture was stirred for 2 hours at room temperature, and then the reaction was quenched by adding water (30 mL). The resulting solution was extracted 3 times with ethyl acetate (30 mL×3), washed 3 times with water and washed once with saturated brine. The organic phase was combined, dried over anhydrous sodium sulfate and concentrated under reduced pressure to obtain a crude product compound, which was subjected to preparative chromatography to obtain compound 130 (30 mg, 8%). Preparative separation conditions: instrument: waters 2767 (preparative liquid phase chromatographic instrument); chromatographic column: XBridge@ Prep C18 (19 mm×250 mm); The sample was dissolved in DMF and filtered with a 0.45 m filter to prepare a sample solution. Preparative chromatography conditions: mobile phase A: acetonitrile; mobile phase B: water (containing 0.5% ammonia water); gradient elution: the content of mobile phase A rises from 45% to 75%; flow rate: 15 ml/min; elution time: 20 min; retention time: about 13 min;

LC-MS (ESI): m/z=598.2 [M+H]⁺.

¹H NMR (400 MHz, DMSO-d6) δ 8.82 (d, 1H), 8.42-8.72 (m, 1H), 8.29-8.39 (m, 2H), 8.15-8.21 (m, 2H), 7.9-8.05 (m, 2H), 7.55 (d, 2H), 7.31-7.45 (m, 3H), 5.41 (s, 1H), 4.23 (s, 3H) 3.45-3.75 (m, 4H), 2.31-2.75 (m, 4H).

### Example 131

### (R/S)-(4-((2-(difluoromethyl)-2H-tetrazol-5-yl)(phenyl)methyl)piperazin-1-yl)(4-(5-(1-methyl-1H-1,2,3-triazol-5-yl)benzo[d]oxazol-2-yl)pyridin-2-yl)methanone

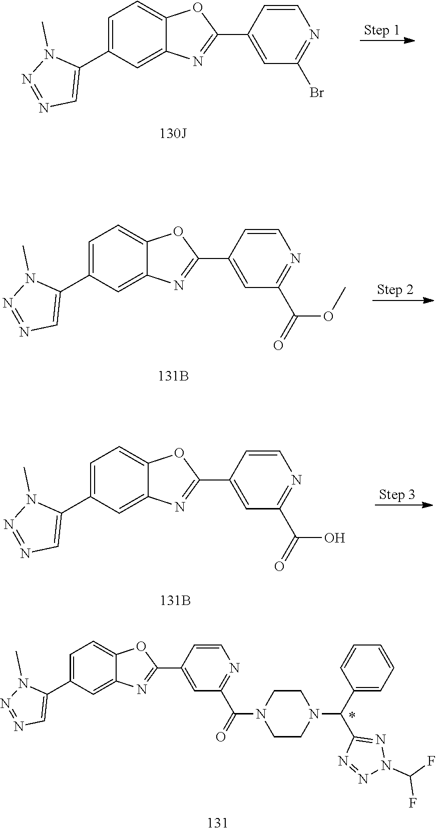 [SMILES: Cn1nncc1-c1ccc2oc(-c3ccnc(Br)c3)nc2c1 ; COC(=O)c1cc(-c2nc3cc(-c4cnnn4C)ccc3o2)ccn1 ; Cn1nncc1-c1ccc2oc(-c3ccnc(C(=O)O)c3)nc2c1 ; Cn1nncc1-c1ccc2oc(-c3ccnc(C(=O)N4CCN(C(c5ccccc5)c5nnn(C(F)F)n5)CC4)c3)nc2c1]

Step 1:

### methyl 4-(5-(1-methyl-1H-1,2,3-triazol-5-yl)benzo[d]oxazol-2-yl)picolinate (131A)

Compound 130J (400 mg, 1.12 mmol), [1,1′-bis(diphenylphosphino)ferrocene]palladium dichloride dichloromethane complex (100 mg) and triethylamine (339 mg, 3.36 mmol) were added to a mixed system of methanol (20 mL) and dichloromethane (20 ml), and the mixture was reacted at 110° C. under carbon monoxide (20 MPa) for 3 hours. The reaction solution was cooled to room temperature and filtered. The filtrate was concentrated under reduced pressure, and then the residue was subjected to column chromatography (EA:PE=20%-60%) to obtain compound 131A (300 mg, 80%).

LC-MS (ESI): m/z=336.3 [M+H]⁺.

Step 2:

### 4-(5-(1-methyl-1H-1,2,3-triazol-5-yl)benzo[d]oxazol-2-yl)picolinic acid (131B)

Compound 131A (300 mg, 0.89 mmol) was added to a mixed system of methanol (20 mL), lithium hydroxide (300 mg) and water (20 ml), and the mixture was reacted at 45° C. for 1 hour. The reaction solution was adjusted to pH=5-6 with 2 M hydrochloric acid and filtered to obtain the filter cake, which was compound 131B (150 mg, 52%).

LC-MS (ESI): m/z=322.1 [M+H]⁺.

Step 3:

### (R/S)-(4-((2-(difluoromethyl)-2H-tetrazol-5-yl)(phenyl)methyl)piperazin-1-yl)(4-(5-(1-methyl-1H-1,2,3-triazol-5-yl)benzo[d]oxazol-2-yl)pyridin-2-yl)methanone (Compound 131)

Intermediate 7 (183 mg, 0.62 mmol) and 131B (150 mg, 0.47 mmol) were dissolved in N,N-dimethylformamide (20 mL). HATU (270 mg, 0.71 mmol) and DIPEA (121 mg, 0.94 mmol) were added. After the addition, the mixture was stirred for 2 hours at room temperature, and then the reaction was quenched by adding water (30 mL). The resulting solution was extracted 3 times with ethyl acetate (30 mL), washed 3 times with water and washed once with saturated brine. The organic phase was combined, dried over anhydrous sodium sulfate and concentrated under reduced pressure to obtain a crude product compound, which was subjected to prep-HPLC to obtain compound 131 (30 mg, 7%). Preparative separation conditions: instrument: waters 2767 (preparative liquid phase chromatographic instrument); chromatographic column: XBridge@ Prep C18 (19 mm×250 mm); The sample was dissolved in DMF and filtered with a 0.45 m filter to prepare a sample solution. Preparative chromatography conditions: mobile phase A: acetonitrile; mobile phase B: water (containing 0.5% ammonia water); gradient elution: the content of mobile phase A rises from 45% to 75%; flow rate: 15 ml/min; elution time: 20 min; retention time: about 13 min.

LC-MS (ESI): m/z=598.2 [M+H]⁺.

¹H NMR (400 MHz, DMSO-d6) δ 8.82 (d, 1H), 8.42-8.72 (m, 1H), 8.29-8.39 (m, 2H), 8.15-8.21 (m, 2H), 7.9-8.05 (m, 2H), 7.50 (d, 2H), 7.31-7.45 (m, 3H), 5.33 (s, 1H), 4.12 (s, 3H) 3.51-3.72 (m, 4H), 2.36-2.85 (m, 4H).

### Example 132

### 3,3,3-trideuterio-N-[2-[2-[4-[(R/S)-(5-methyltetrazol-2-yl)-phenyl-methyl]piperidine-1-carbonyl]-4-pyridyl]-1,3-benzoxazol-5-yl]-2-(trideuteriomethyl)propanamide

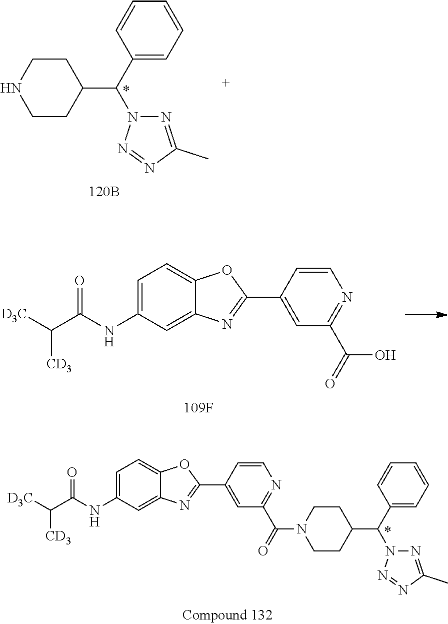 [SMILES: Cc1nnn(C(c2ccccc2)C2CCNCC2)n1 ; [2H]C([2H])([2H])C(C(=O)Nc1ccc2oc(-c3ccnc(C(=O)O)c3)nc2c1)C([2H])([2H])[2H] ; [2H]C([2H])([2H])C(C(=O)Nc1ccc2oc(-c3ccnc(C(=O)N4CCC(C(c5ccccc5)n5nnc(C)n5)CC4)c3)nc2c1)C([2H])([2H])[2H]]

Compounds 120B and 109F were used as raw materials to obtain compound 132 according to the synthetic method of compound 85.

LC-MS (ESI): m/z=571.3[M+H]⁺.

¹H NMR (400 MHz, CDCl₃) δ 8.73-8.70 (m, 1H), 8.31 (s, 1H), 8.08-8.05 (m, 1H), 8.00-7.99 (m, 1H), 7.55-7.48 (m, 4H), 7.40-7.33 (m, 3H), 5.53-5.51 (m, 2H), 4.78-4.76 (m, 1H), 3.97-3.94 (m, 1H), 3.10-3.07 (m, 1H), 2.88-2.81 (m, 2H), 2.55-2.50 (m, 3H), 1.45-1.36 (m, 3H), 1.27-1.24 (m, 2H).

### Example 133

### (R/S)-(4-(5-(1-methyl-1H-pyrazol-4-yl)benzo[d]oxazol-2-yl)pyridin-2-yl)(4-((5-methyl-2H-tetrazol-2-yl)(phenyl)methyl)piperidin-1-yl)methanone

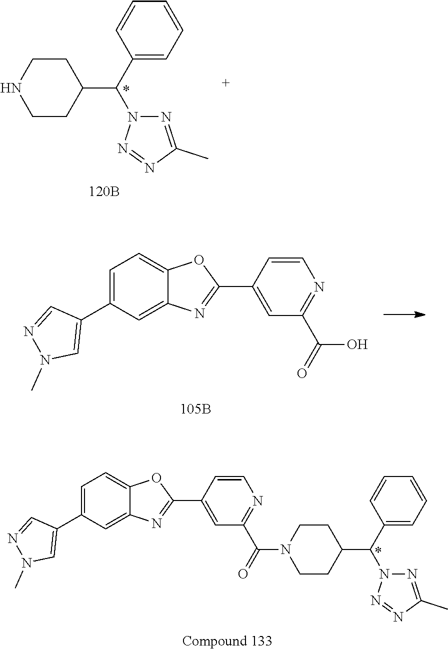 [SMILES: Cc1nnn(C(c2ccccc2)C2CCNCC2)n1 ; Cn1cc(-c2ccc3oc(-c4ccnc(C(=O)O)c4)nc3c2)cn1 ; Cc1nnn(C(c2ccccc2)C2CCN(C(=O)c3cc(-c4nc5cc(-c6cnn(C)c6)ccc5o4)ccn3)CC2)n1]

Compounds 120B and 105B were used as raw materials to obtain compound 133 according to the synthetic method of compound 85.

LC-MS (ESI): m/z=560.3[M+H]⁺.

¹H NMR (400 MHz, CDCl₃) δ 8.75-8.73 (m, 1H), 8.40 (s, 1H), 8.13-8.11 (m, 1H), 7.88-7.87 (m, 1H), 7.82 (s, 1H), 7.67 (s, 1H), 7.62-7.50 (m, 4H), 7.42-7.33 (m, 3H), 5.51-5.51 (m, 1H), 4.78-4.76 (m, 1H), 3.96-4.00 (m, 4H), 3.16-3.08 (m, 1H), 2.93-2.81 (m, 2H), 2.55-2.50 (m, 3H), 1.59-1.26 (m, 4H).

### Example 134

### (1S,2S)-2-fluoro-N-(2-(2-(4-((R/S)-(5-methyl-2H-tetrazol-2-yl)(phenyl)methyl)piperidine-1-carbonyl)pyridin-4-yl)benzo[d]oxazol-6-yl)cyclopropane-1-carboxamide

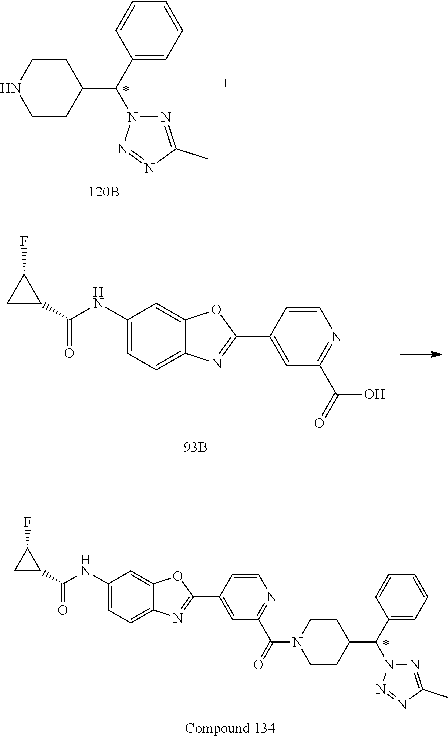

Compounds 120B and 93B were used as raw materials to obtain compound 134 according to the synthetic method of compound 85.

LC-MS (ESI): m/z=581.2[M+H]⁺.

¹H NMR (400 MHz, CDCl₃) δ 8.73 (s, 1H), 8.35-8.32 (m, 2H), 8.15 (s, 1H), 7.72-7.70 (m, 2H), 7.53-7.50 (m, 2H), 7.40-7.33 (m, 3H), 7.20-7.18 (m, 1H), 5.55-5.52 (m, 1H), 4.77-4.73 (m, 2H), 3.82-3.80 (m, 2H), 3.21-3.19 (m, 2H), 2.88-2.85 (m, 2H), 2.55-2.49 (m, 3H), 1.27-1.25 (m, 5H).

### Example 135

### (R/S)-2-fluoro-2-methyl-N-(2-(2-(4-((5-methyl-2H-tetrazol-2-yl)(phenyl)methyl)piperidine-1-carbonyl)pyridin-4-yl)benzo[d]oxazol-5-yl)propanamide

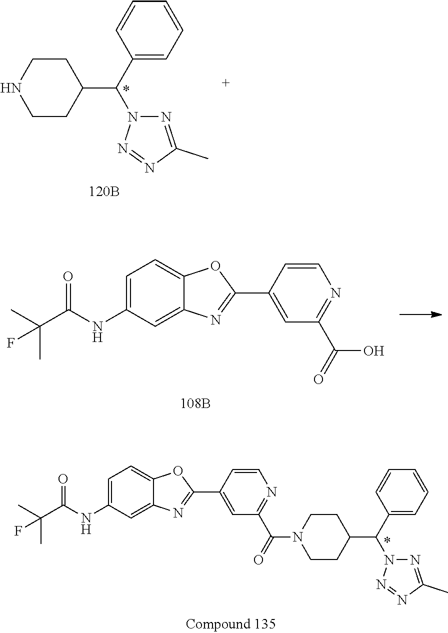 [SMILES: Cc1nnn(C(c2ccccc2)C2CCNCC2)n1 ; CC(C)(F)C(=O)Nc1ccc2oc(-c3ccnc(C(=O)O)c3)nc2c1 ; Cc1nnn(C(c2ccccc2)C2CCN(C(=O)c3cc(-c4nc5cc(NC(=O)C(C)(C)F)ccc5o4)ccn3)CC2)n1]

Compounds 120B and 108B were used as raw materials to obtain compound 135 according to the synthetic method of compound 85.

LC-MS (ESI): m/z=583.3[M+H]⁺.

¹H NMR (400 MHz, CDCl₃) δ 8.75 (s, 1H), 8.42 (s, 1H), 8.25-8.23 (m, 1H), 8.17-8.16 (m, 2H), 7.62-7.57 (m, 2H), 7.55-7.50 (m, 2H), 7.40-7.31 (m, 3H), 5.54-5.52 (m, 1H), 4.77-4.75 (m, 1H), 3.90-3.86 (m, 1H), 2.90-2.80 (m, 2H), 2.55-2.50 (m, 3H), 2.49-2.38 (m, 2H), 1.73 (s, 3H), 1.68 (s, 3H), 0.9-0.83 (m, 3H).

### Example 136

### 3,3,3-trideuterio-N-[2-[2-[4-[(R)-[2-(difluoromethyl)tetrazol-5-yl]-phenyl-methyl]piperazine-1-carbonyl]-4-pyridyl]benzofuran-5-yl]-2-(trideuteriomethyl)propanamide

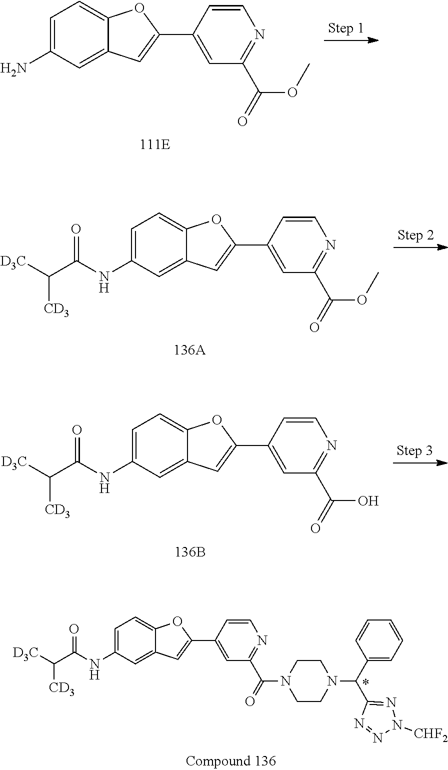 [SMILES: COC(=O)c1cc(-c2cc3cc(N)ccc3o2)ccn1 ; [2H]C([2H])([2H])C(C(=O)Nc1ccc2oc(-c3ccnc(C(=O)OC)c3)cc2c1)C([2H])([2H])[2H] ; [2H]C([2H])([2H])C(C(=O)Nc1ccc2oc(-c3ccnc(C(=O)O)c3)cc2c1)C([2H])([2H])[2H] ; [2H]C([2H])([2H])C(C(=O)Nc1ccc2oc(-c3ccnc(C(=O)N4CCN(C(c5ccccc5)c5nnn(C(F)F)n5)CC4)c3)cc2c1)C([2H])([2H])[2H]]

Step 1:

### methyl-4-[5-[[3,3,3-trideuterio-2-(trideuteriomethyl)propanoyl]amino]benzofuran-2-yl]pyridine-2-carboxylate (136A)

3,3,3-trideuterio-2-(trideuteriomethyl)propanoic acid (118 mg, 1.2 mmol) and 111E (268 mg, 1.0 mmol) were dissolved in dichloromethane (10 mL) at room temperature; then HATU (570 mg, 1.5 mmol) and DIPEA (260 mg, 2.0 mmol) were added; and the mixture was stirred at room temperature for another 1 h. The reaction solution was subjected to rotary evaporation and then directly subjected to column chromatography (DCM:MeOH=30:1) to obtain compound 136A (300 mg, yield: 87.2%).

LC-MS (ESI): m/z=345.3[M+H]⁺.

Step 2:

### 4-[5-[[3,3,3-trideuterio-2-(trideuteriomethyl)propanoyl]amino]benzofuran-2-yl]pyridine-2-carboxylic Acid (136B)

Compound 136A (300 mg, 0.87 mmol) and LiOH H₂O (2.6 mmol) were added to THF/MeOH/H₂O (4:1:1, 12 mL) at room temperature, and the mixture was stirred at room temperature overnight. Low boiling point solvent was removed by rotary evaporation, and the solution was acidified to pH=3-4 with 1 mol/L hydrochloric acid. The solid was precipitated. The resulting solution was filtered by suction and washed with water (5 mL×2), and the filter cake was dried in vacuo to obtain compound 136B (210 mg, yield: 70%).

LC-MS (ESI): m/z=331.1[M+H]⁺.

Step 3:

### 3,3,3-trideuterio-N-[2-[2-[4-[(R/S)-[2-(difluoromethyl)tetrazol-5-yl]-phenyl-methyl]piperazine-1-carbonyl]-4-pyridyl]benzofuran-5-yl]-2-(trideuteriomethyl)propanamide (Compound 136)

Compound 136B and intermediate 7 were used as raw materials to obtain compound 136 according to the synthetic method of compound 1.

LC-MS (ESI): m/z=607.3[M+H]⁺.

¹H NMR (400 MHz, CDCl₃) δ 8.59-8.57 (m, 1H), 8.03-8.02 (m, 1H), 7.77-7.59 (m, 4H), 7.48-7.44 (m, 1H), 7.41-7.34 (m, 3H), 7.31-7.30 (m, 2H), 7.22 (s, 1H), 5.30 (s, 1H), 3.98-3.84 (m, 4H), 2.89-2.81 (m, 2H), 2.70-2.59 (m, 2H), 2.51 (s, 1H).

### Example 137

### 3,3,3-trideuterio-N-[2-[2-[4-[(R/S)-(5-methyltetrazol-2-yl)-phenyl-methyl]piperidine-1-carbonyl]-4-pyridyl]benzofuran-5-yl]-2-(trideuteriomethyl)propanamide

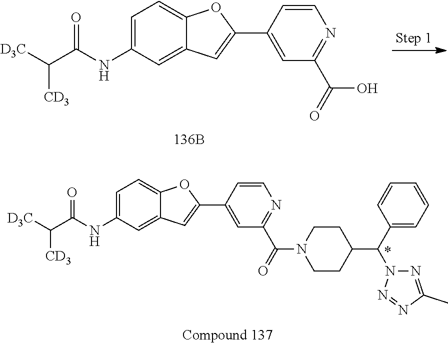 [SMILES: [2H]C([2H])([2H])C(C(=O)Nc1ccc2oc(-c3ccnc(C(=O)O)c3)cc2c1)C([2H])([2H])[2H] ; [2H]C([2H])([2H])C(C(=O)Nc1ccc2oc(-c3ccnc(C(=O)N4CCC(C(c5ccccc5)n5nnc(C)n5)CC4)c3)cc2c1)C([2H])([2H])[2H]]

Compounds 136B and 120B were used as raw materials to obtain compound 137 according to the synthetic method of compound 1.

LC-MS (ESI): m/z=570.3[M+H]⁺.

¹H NMR (400 MHz, CDCl₃) δ 8.59-8.57 (m, 1H), 8.03 (s, 1H), 7.98 (s, 1H), 7.73-7.71 (m, 1H), 7.55-7.50 (m, 2H), 7.45-7.29 (m, 5H), 7.23-7.21 (m, 1H), 5.54-5.51 (m, 1H), 4.76-4.74 (m, 1H), 3.96-3.92 (m, 1H), 3.15-3.10 (m, 1H), 2.89-2.78 (m, 2H), 2.55-2.49 (m, 4H), 1.58-1.39 (m, 4H).

### Example 138

### (1S,2S)—N-(2-(2-(4-((R/S)-(5-(difluoromethyl)-2H-tetrazol-2-yl)(phenyl)methyl)piperidine-1-carbonyl)pyridin-4-yl)benzo[d]oxazol-5-yl)-2-fluorocyclopropanecarboxamide

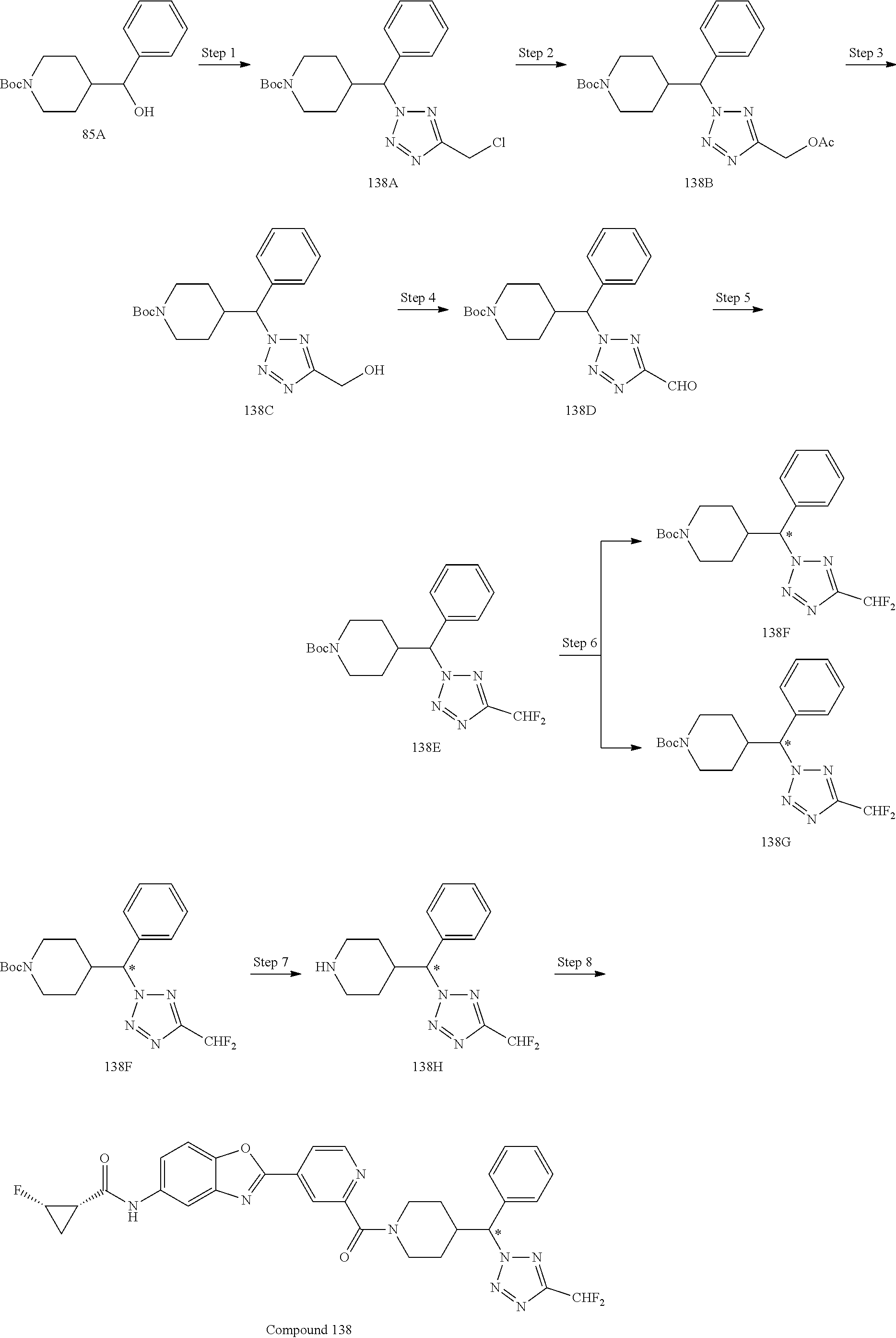

Step 1:

### tert-butyl-4-((5-(chloromethyl)-2H-tetrazol-2-yl)(phenyl)methyl)piperidine-1-carboxylate (138A)

At room temperature, compound 85A (8.61 g, 30.0 mmol), 5-chloromethyltetrazole (3.80 g, 32.0 mmol) and triphenylphosphine (11.79 g, 45.0 mmol) were dissolved in anhydrous THE (100 mL); the mixture was cooled to 0° C. under nitrogen protection; and then DIAD (diisopropyl azodicarboxylate) (9.10 g, 45.0 mmol) was added dropwise. The mixture was allowed to naturally warm to room temperature, reacted overnight, concentrated under reduced pressure and subjected to column chromatography (PE:EA=10:1) to obtain compound 138A (5.70 g, 50.0%).

LC-MS (ESI): m/z=392.2[M+H]⁺.

Step 2:

### 4-((5-(acetoxymethyl)-2H-tetrazol-2-yl)(phenyl)methyl)piperidine-1-carboxylate (138B)

Compound 138A (4.70 g, 12.0 mmol) was dissolved in acetonitrile (100 mL) at room temperature, and then potassium acetate (1.77 g, 18.0 mmol) and benzyltrimethylammonium bromide (2.76 g, 12.0 mmol) was added. The mixture was stirred at 80° C. for 1 hour, and the reaction solution was subjected to rotary evaporation to obtain a crude product of compound 138B (5.00 g), which was directly used in the next reaction without purification.

LC-MS (ESI): m/z=416.3[M+H]⁺.

Step 3:

### 4-((5-(hydroxymethyl)-2H-tetrazol-2-yl)(phenyl)methyl)piperidine-1-carboxylate (138C)

The crude product of 138B (5.00 g, 12.0 mmol) was dissolved in methanol (100 mL) at room temperature; potassium carbonate (3.31 g, 24.0 mmol) was added; and the mixture was stirred at room temperature for 2 hours. The resulting solution was filtered, and the mother liquor was subjected to rotary evaporation to obtain a crude product of compound 138C (4.70 g), which was directly used in the next reaction without purification.

LC-MS (ESI): m/z=374.3[M+H]⁺.

Step 4:

### tert-butyl 4-((5-formyl-2H-tetrazol-2-yl)(phenyl)methyl)piperidine-1-carboxylate (138D)

The crude product of 138C (4.70, 12.0 mmol) was dissolved in dichloromethane (50 mL); Dess-Martin reagent (15.26 g, 36.0 mmol) was added at room temperature; and the mixture was stirred overnight. The reaction was quenched by adding 20% sodium thiosulfate (50 mL), neutralized to a basic pH with saturated sodium bicarbonate and extracted with dichloromethane (50 mL×3). The organic phase was combined, washed with water (50 mL×1) and saturated sodium chloride solution (50 mL×1), dried over anhydrous sodium sulfate and then concentrated under reduced pressure to obtain a crude product of compound 138D (5.00 g), which was directly used in the next reaction without purification.

LC-MS (ESI): m/z=404.3[M+MeOH+H]⁺.

¹H NMR (400 MHz, CDCl₃) δ 10.22 (s, 1H), 7.56-7.53 (m, 2H), 7.42-7.26 (m, 3H), 5.66-5.63 (m, 1H), 4.08-4.07 (m, 2H), 2.81-2.67 (m, 3H), 1.43 (s, 9H), 1.27-1.13 (m, 4H).

Step 5:

### tert-butyl 4-((5-(difluoromethyl)-2H-tetrazol-2-yl)(phenyl)methyl)piperidine-1-carboxylate (138E)

The crude product of 138D (5.00 g, 13.5 mmol) from step 4 was added to anhydrous dichloromethane (50 mL). DAST (diethylaminosulphur trifluoride) (6.51 g, 40.4 mmol) was slowly added dropwise at 0° C. After the addition, the mixture was warmed to room temperature and reacted for 2 hours. The reaction was quenched by adding 10 mL of methanol. The reaction solution was subjected to rotary evaporation and then directly subjected to column chromatography (PE:EA=8:1) to obtain compound 138E (2.50 g, total yield for four steps: 53%).

¹H NMR (400 MHz, CDCl₃) δ 7.54-7.52 (m, 2H), 7.38-7.40 (m, 3H), 6.92 (t, J=52.8 Hz, 1H), 5.60-5.58 (m, 1H), 4.10-4.08 (m, 2H), 2.76-2.66 (m, 3H), 1.56-1.26 (m, 11H), 1.22-1.14 (m, 2H).

¹⁹FNMR (376 MHz, CDCl₃) δ−114.91 (s, 2F).

Step 6:

### (R/S)-tert-butyl 4-((5-(difluoromethyl)-2H-tetrazol-2-yl)(phenyl)methyl)piperidine-1-carboxylate (138F)

### (S/R)-tert-butyl 4-((5-(difluoromethyl)-2H-tetrazol-2-yl)(phenyl)-methyl)piperidine-1-carboxylate (138G)

Compound 138E (2.50 g) obtained from step 5 was subjected to chiral preparative, and the method was as follows:

instrument: Gilson GX-281 (preparative liquid phase chromatographic instrument); chromatographic column: CHIRALPAKAD-H, 5 μm, 20 mm×250 mm; preparation of samples: The sample was dissolved in ethanol and filtered with a 0.45 m filter to prepare a sample solution. Preparative chromatography conditions: mobile phase system: n-hexane/isopropanol (10%); isocratic elution; elution time: 26 min; and flow rate: 9.5 ml/min;

The samples were collected and concentrated to obtain 138F (retention time: 9.7 min, 1.10 g) and 138G (retention time: 11.3 min, 1.10 g).

Step 7:

### (R/S)-4-((5-(difluoromethyl)-2H-tetrazol-2-yl)(phenyl)methyl)piperidine (138H)

Compound 138F (1.10 g, 2.8 mmol) was dissolved in dichloromethane (10 mL) at room temperature, and then trifluoroacetic acid (2.5 mL, 33.7 mmol) was added dropwise. After the dropwise addition was completed, the mixture was stirred at room temperature for another 2 h. The reaction solution was subjected to rotary evaporation, and the residue was dissolved in dichloromethane (20 mL), neutralized to a basic pH with saturated NaHCO₃, washed with water (10 mL×2) and saturated sodium chloride solution (10 mL×1), dried over anhydrous sodium sulfate, filtered and concentrated to obtain a crude product of 138H (0.61 g, yield: 74%).

LC-MS (ESI): m/z=294.2[M+H].

Step 8:

### (1S,2S)—N-(2-(2-(4-((R/S)-(5-(difluoromethyl)-2H-tetrazol-2-yl)(phenyl)methyl)piperidine-1-carbonyl)pyridin-4-yl)benzo[d]oxazol-5-yl)-2-fluorocyclopropanecarboxamide (Compound 138)

Compounds 117F and 138H were used as raw materials to obtain compound 138 according to the synthetic method of compound 1.

LC-MS (ESI): m/z=617.2[M+H]⁺.

¹H NMR (400 MHz, CDCl₃) δ 8.72-8.70 (m, 1H), 8.25-8.16 (m, 2H), 8.06-8.03 (m, 1H), 7.96 (s, 1H), 7.57-7.48 (m, 3H), 7.45-7.34 (m, 4H), 7.08-6.76 (m, 1H), 5.68-5.64 (m, 1H), 4.88-4.69 (m, 2H), 3.96-3.93 (m, 1H), 3.16-3.06 (m, 1H), 2.98-2.79 (m, 1H), 2.08-2.06 (m, 1H), 1.92-1.85 (m, 2H), 1.62-1.38 (m, 3H), 1.30-1.19 (m, 2H).

### Example 139

### (1S,2S)-2-fluoro-N-(2-(2-(4-((R)-(5-methyl-2H-tetrazol-2-yl)(phenyl)methyl)piperidine-1-carbonyl)pyridin-4-yl)benzofuran-5-yl)cyclopropanecarboxamide

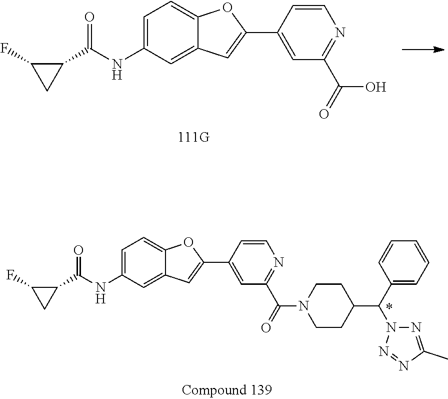 [SMILES: O=C(O)c1cc(-c2cc3cc(NC(=O)[C@@H]4C[C@@H]4F)ccc3o2)ccn1 ; Cc1nnn(C(c2ccccc2)C2CCN(C(=O)c3cc(-c4cc5cc(NC(=O)[C@@H]6C[C@@H]6F)ccc5o4)ccn3)CC2)n1]

111G and 120B were used as raw materials to obtain compound 139 according to the synthetic step of compound 1.

LC-MS (ESI): m/z=580.2 [M+H]⁺.

¹H NMR (400 MHz, CDCl₃) δ 8.58 (s, 1H), 7.99 (s, 1H), 7.92 (s, 1H), 7.79 (s, 1H), 7.70 (s, 1H), 7.58-7.48 (m, 2H), 7.46-7.30 (m, 4H), 7.28-7.22 (m, 1H), 7.15 (s, 1H), 5.52 (d, J=10.8, 1H), 4.82-4.70 (m, 2H), 3.92 (s, 1H), 3.19-3.10 (m, 1H), 2.96-2.76 (m, 2H), 2.52 (d, J=21.0 Hz, 3H), 1.92-1.75 (m, 2H), 1.63-1.53 (m, 1H) 1.50-1.30 (m, 3H), 1.29-1.18 (m, 1H).

### Example 140

### (R/S)-(4-(5-(isothiazol-4-yl)benzo[d]oxazol-2-yl)pyridin-2-yl)(4-((5-methyl-2H-tetrazol-2-yl)(phenyl)methyl)piperidin-1-yl)methanone

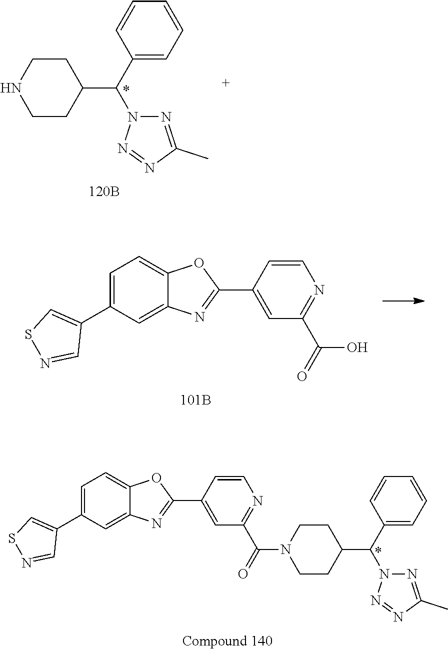 [SMILES: Cc1nnn(C(c2ccccc2)C2CCNCC2)n1 ; O=C(O)c1cc(-c2nc3cc(-c4cnsc4)ccc3o2)ccn1 ; Cc1nnn(C(c2ccccc2)C2CCN(C(=O)c3cc(-c4nc5cc(-c6cnsc6)ccc5o4)ccn3)CC2)n1]

Compounds 120B and 101B were used as raw materials to obtain compound 140 according to the synthetic method of compound 1.

LC-MS (ESI): m/z=563.2[M+H]⁺.

¹H NMR (400 MHz, CDCl₃) δ 8.59 (s, 1H), 7.92 (s, 1H), 7.78-7.71 (m, 3H), 7.63 (s, 1H), 7.63 (s, 1H), 7.55-7.47 (m, 3H), 7.41-7.31 (m, 3H), 5.54-5.51 (m, 1H), 3.17-3.10 (m, 2H), 2.89-2.81 (m, 2H), 2.55-2.50 (m, 3H), 1.30-1.26 (m, 5H).

### Example 141

### (R/S)—N-(2-(2-(4-((2-(difluoromethyl)-2H-tetrazol-5-yl)(phenyl)methyl)piperazine-1-carbonyl)pyridin-4-yl)benzofuran-5-yl)-2-fluoro-2-methylpropanamide

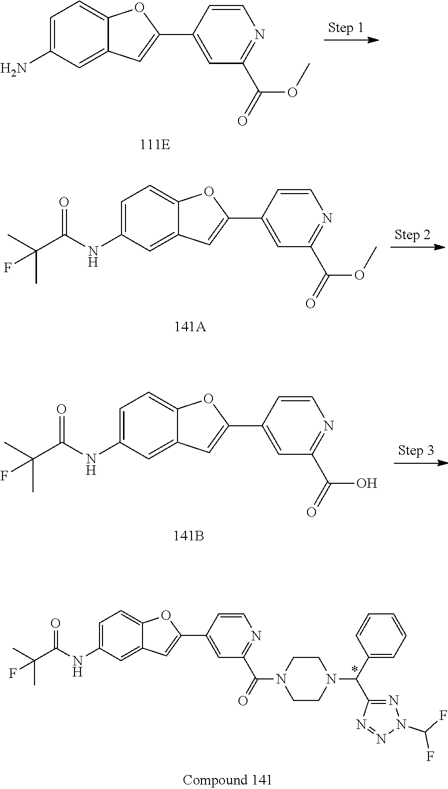

Step 1:

### 4-(5-(2-fluoro-2-methylpropanamido)benzofuran-2-yl)picolinate (141A)

Monofluoroisobutyric acid (127 mg, 1.2 mmol), DMF (10 mL), DIPEA (260 mg, 2 mmol), HATU (456 mg, 1.2 mmol) and compound 111E (268 mg, 1 mmol) were successively added to a single-necked flask, and the mixture was stirred at room temperature for 3 h. The reaction was quenched by adding aqueous solution (30 mL), extracted with dichloromethane (50 mL×1) and allowed to stand for phase separation. The aqueous phase was extracted with dichloromethane (100 mL×2). The combined organic phase was dried over anhydrous sodium sulfate and concentrated under reduced pressure to obtain a crude product from the residue, and the crude product was separated by column chromatography (PE:EA=3:1) to obtain compound 141A (300 mg, 84.27%).

LC-MS (ESI): m/z=357.1[M+H]⁺.

Step 2:

### 4-(5-(2-fluoro-2-methylpropanamido)benzofuran-2-yl)picolinic Acid (141B)

Compound 141A (300 mg, 0.84 mmol), tetrahydrofuran (2 mL), methanol (5 ml), distilled water (2 mL) and lithium hydroxide monohydrate (300 mg, 7 mmol) were successively added to a single-necked flask, and the mixture was stirred at room temperature for 3 h. The reaction was quenched by adding dilute hydrochloric acid and extracted with dichloromethane (50 mL×2). The combined organic phase was dried over anhydrous sodium sulfate and concentrated under reduced pressure to obtain a crude product of compound 141B (280 mg, 97.56%) from the residue. The crude product can be directly used in the next reaction.

LC-MS (ESI): m/z=343.1[M+H]⁺.

Step 3:

### (R/S)—N-(2-(2-(4-((2-(difluoromethyl)-2H-tetrazol-5-yl)(phenyl)methyl)piperazine-1-carbonyl)pyridin-4-yl)benzofuran-5-yl)-2-fluoro-2-methylpropanamide

Under nitrogen protection, compound 141B (80 mg, 0.23 mmol), DMF (10 mL), HATU (137 mg, 0.36 mmol), DIPEA (58 mg, 0.45 mmol) and intermediate 7 (106 mg, 0.36 mmol) were successively added to a single-necked flask, and the mixture was stirred at room temperature for 3 h. The reaction was quenched by adding aqueous solution (30 mL), extracted with dichloromethane (50 mL×1) and allowed to stand for phase separation. The aqueous phase was washed with dichloromethane (100 mL×2). The combined organic phase was dried over anhydrous sodium sulfate and concentrated under reduced pressure to obtain a crude product from the residue, and the crude product was separated by preparative high-performance liquid chromatography to obtain compound 141 as a light yellow solid (50 mg, 35.2%). Preparative separation conditions: instrument: waters 2767 (preparative liquid phase chromatographic instrument); chromatographic column: XBridge@ Prep C18 (19 mm×250 mm); The sample was dissolved in DMF and filtered with a 0.45 m filter to prepare a sample solution. Preparative chromatography conditions: mobile phase A: acetonitrile; mobile phase B: water (containing 0.5% ammonia water); gradient elution: the content of mobile phase A rises from 45% to 75%; flow rate: 15 ml/min; elution time: 20 min; retention time: about 15.47 min;

¹H NMR (400 MHz, CDCl₃) δ 8.60-8.59 (m, 1H), 8.19-8.15 (m, 1H), 8.08-8.06 (m, 2H), 7.77-7.72 (m, 1H), 7.63-7.60 (m, 2H), 7.51-7.42 (m, 2H), 7.40-7.35 (m, 4H), 5.15 (s, 1H) 3.99-3.86 (m, 4H), 2.89-2.67 (m, 4H), 1.72 (s, 3H), 1.67 (s, 3H).

LC-MS (ESI): m/z=619.2 [M+H]⁺.

### Example 142

### (R/S)-2-fluoro-2-methyl-N-(2-(2-(4-((5-methyl-2H-tetrazol-2-yl)(phenyl)methyl)piperidine-1-carbonyl)pyridin-4-yl)benzofuran-5-yl)propanamide

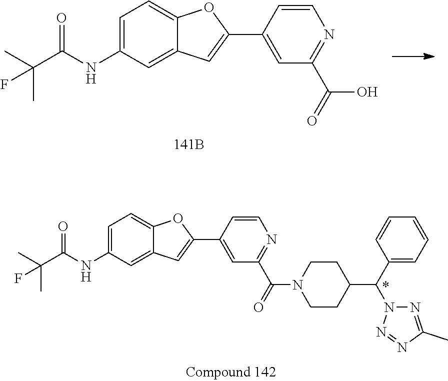 [SMILES: CC(C)(F)C(=O)Nc1ccc2oc(-c3ccnc(C(=O)O)c3)cc2c1 ; Cc1nnn(C(c2ccccc2)C2CCN(C(=O)c3cc(-c4cc5cc(NC(=O)C(C)(C)F)ccc5o4)ccn3)CC2)n1]

Compounds 141B and 120B were used as raw materials to obtain compound 142 according to the synthetic method of compound 2.

¹H NMR (400 MHz, CDCl₃) δ 8.61-8.59 (m, 1H), 8.20-8.11 (m, 1H), 8.08-8.07 (m, 2H), 8.01 (s, 1H), 7.73-7.70 (m, 1H), 7.55-7.49 (m, 2H), 7.41-7.29 (m, 4H), 7.25 (s, 1H), 5.53-5.50 (m, 1H) 4.05-4.02 (m, 1H), 3.13-3.02 (m, 2H), 2.87-2.76 (m, 2H), 2.55-2.50 (m, 3H), 1.72 (s, 3H), 1.67 (s, 3H), 1.29-1.25 (m, 4H)

LC-MS (ESI): m/z=582.3 [M+H]⁺.

### Example 143

### (R/S)—N-(2-(2-(4-((2-(difluoromethyl)-2H-tetrazol-5-yl)(phenyl)methyl)piperazine-1-carbonyl)pyridin-4-yl)benzofuran-5-yl)-1-fluorocyclopropane-1-carboxamide

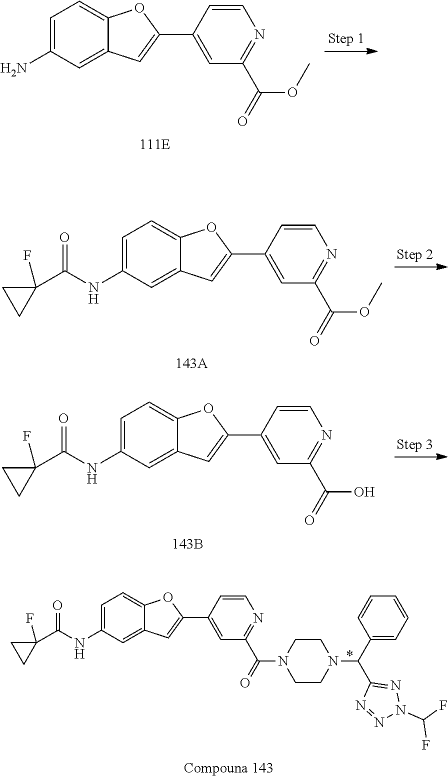 [SMILES: COC(=O)c1cc(-c2cc3cc(N)ccc3o2)ccn1 ; COC(=O)c1cc(-c2cc3cc(NC(=O)C4(F)CC4)ccc3o2)ccn1 ; O=C(O)c1cc(-c2cc3cc(NC(=O)C4(F)CC4)ccc3o2)ccn1 ; O=C(c1cc(-c2cc3cc(NC(=O)C4(F)CC4)ccc3o2)ccn1)N1CCN(C(c2ccccc2)c2nnn(C(F)F)n2)CC1]

Compounds 1-fluorocyclopropanecarboxylic acid and 111E were used as raw materials to obtain compound 143B according to the synthetic method of compound 141.

Compound 143B and intermediate 7 were used as raw materials to obtain compound 143 according to the synthetic method of compound 2.

¹H NMR (400 MHz, CDCl₃) δ 8.61-8.59 (m, 1H), 8.19-8.17 (m, 1H), 8.06-8.05 (m, 2H), 7.77-7.72 (m, 1H), 7.63-7.59 (m, 2H), 7.51-7.48 (m, 1H), 7.42-7.33 (m, 4H), 7.25-7.24 (m, 1H), 5.3 (s, 1H) 3.96 (s, 1H), 3.13-3.02 (m, 2H), 2.87-2.76 (m, 2H), 1.55-1.49 (m, 4H), 1.45-1.36 (m, 2H), 1.30-1.25 (m, 2H).

LC-MS (ESI): m/z=617.2 [M+H]⁺.

### Example 144

### (R/S)-2-fluoro-2-methyl-N-(2-(2-(4-((5-methyl-2H-tetrazol-2-yl)(phenyl)methyl)piperidine-1-carbonyl)pyridin-4-yl)benzofuran-5-yl)propanamide

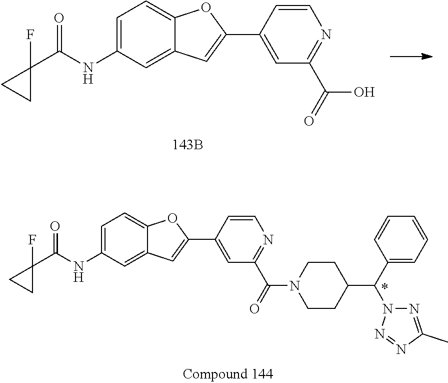 [SMILES: O=C(O)c1cc(-c2cc3cc(NC(=O)C4(F)CC4)ccc3o2)ccn1 ; Cc1nnn(C(c2ccccc2)C2CCN(C(=O)c3cc(-c4cc5cc(NC(=O)C6(F)CC6)ccc5o4)ccn3)CC2)n1]

Compounds 143B and 120B were used as raw materials to obtain compound 144 according to the synthetic method of compound 2.

¹H NMR (400 MHz, CDCl₃) δ 8.61 (s, 1H), 8.21-8.19 (m, 1H), 8.08-8.04 (m, 2H), 7.79 (s, 1H), 7.55-7.50 (m, 3H), 7.42-7.31 (m, 5H), 5.54-5.53 (m, 1H), 4.75 (s, 1H), 3.23-3.16 (m, 2H), 2.90-2.79 (m, 2H), 2.55-2.50 (m, 3H), 1.55-1.49 (m, 2H), 1.47-1.37 (m, 4H), 1.30-1.27 (m, 2H).

LC-MS (ESI): m/z=580.2 [M+H]⁺.

### Example 145

### (R/S)-2-fluoro-2-methyl-N-(2-(2-(4-((5-methyl-2H-tetrazol-2-yl)(phenyl)methyl)piperidine-1-carbonyl)pyridin-4-yl)benzofuran-5-yl)propenamide

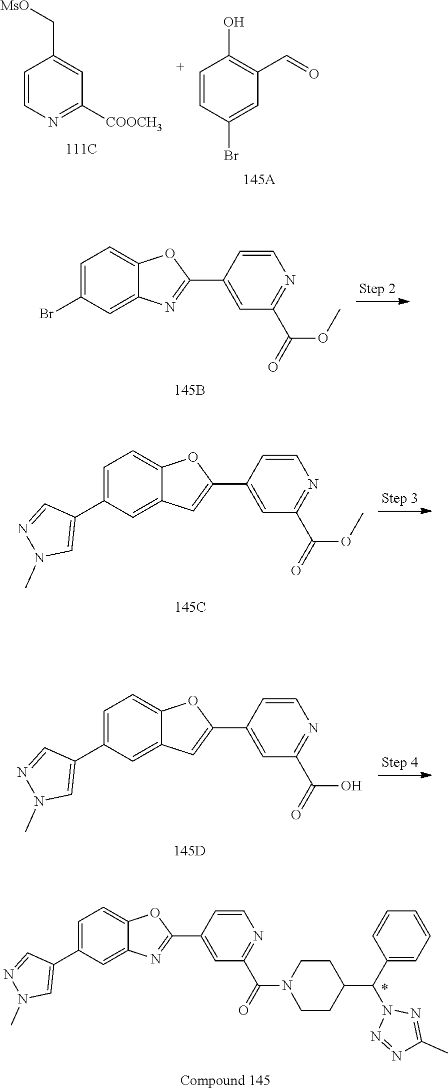 [SMILES: COC(=O)c1cc(COS(C)(=O)=O)ccn1 ; O=Cc1cc(Br)ccc1O ; COC(=O)c1cc(-c2nc3cc(Br)ccc3o2)ccn1 ; COC(=O)c1cc(-c2cc3cc(-c4cnn(C)c4)ccc3o2)ccn1 ; Cn1cc(-c2ccc3oc(-c4ccnc(C(=O)O)c4)cc3c2)cn1 ; Cc1nnn(C(c2ccccc2)C2CCN(C(=O)c3cc(-c4nc5cc(-c6cnn(C)c6)ccc5o4)ccn3)CC2)n1]

Step 1:

### Methyl 6-(5-bromobenzofuran-2-yl)picolinate (145B)

Under nitrogen protection, 111C (1.34 g, 5.47 mmol), DMF (15 mL), 2-hydroxyl-5-bromo-benzaldehyde (1.1 g, 5.47 mmol) and K₂CO₃ (2.07 g, 15 mmol) were successively added to a single-necked flask, and the mixture was refluxed with stirring at 85° C. overnight. The reaction was quenched by adding saturated aqueous sodium bicarbonate solution (30 mL), extracted with ethyl acetate (50 mL×1) and allowed to stand for phase separation. The aqueous phase was washed with ethyl acetate (100 mL×2). The combined organic phase was dried over anhydrous sodium sulfate and concentrated under reduced pressure to obtain a crude product from the residue, and the crude product was separated by column chromatography (PE:EA=10:1) to obtain compound 145B (1.4 g, 74%).

LC-MS (ESI): m/z=332.0 [M+H]⁺.

Step 2:

### Methyl 4-(5-(1-methyl-1H-pyrazol-4-yl)benzofuran-2-yl)picolinate (145C)

Under nitrogen protection, 145B (331 mg, 1 mmol), dioxane (20 mL), 1-methylpyrazole-4-boronic acid pinacol ester (312 mg, 1.5 mmol) and K₂CO₃ (276 mg, 2 mmol) were successively added to a single-necked flask, and finally Pd(dppf)Cl₂ (73.2 mg, 0.1 mmol) and 1 ml of water were added. The reaction was refluxed with stirring at 100° C. for 4 h. The reaction was quenched by adding saturated aqueous sodium bicarbonate solution (30 mL), extracted with DCM (50 mL×1) and allowed to stand for phase separation. The aqueous phase was washed with dichloromethane (100 mL×2). The combined organic phase was dried over anhydrous sodium sulfate and concentrated under reduced pressure to obtain a crude product from the residue, and the crude product was separated by column chromatography (PE:EA=5:1) to obtain compound 145C (220 mg, 88%).

LC-MS (ESI): m/z=334.1 [M+H]⁺.

Step 3:

### 4-(5-(1-methyl-1H-pyrazol-4-yl)benzofuran-2-yl)picolinic Acid (145D)

Compound 145C (220 mg, 0.66 mmol), tetrahydrofuran (2 mL), methanol (5 ml), distilled water (2 mL) and lithium hydroxide monohydrate (300 mg, 7 mmol) were successively added to a single-necked flask, and the mixture was stirred at room temperature for 3 h. The reaction was quenched by adding dilute hydrochloric acid and extracted with dichloromethane (50 mL×2). The combined organic phase was dried over anhydrous sodium sulfate and concentrated under reduced pressure to obtain a crude product of compound 145D (200 mg, 95%) from the residue. The crude product can be directly used in the next reaction.

LC-MS (ESI): m/z=320.1[M+H]⁺.

Step 4:

### (R/S)-2-fluoro-2-methyl-N-(2-(2-(4-((5-methyl-2H-tetrazol-2-yl)(phenyl)methyl)piperidine-1-carbonyl)pyridin-4-yl)benzofuran-5-yl)propenamide (Compound 145)

Under nitrogen protection, compound 145D (80 mg, 0.25 mmol), DMF (10 mL), HATU (137 mg, 0.36 mmol), DIPEA (58 mg, 0.45 mmol) and 120B (106 mg, 0.41 mmol) were successively added to a single-necked flask, and the mixture was stirred at room temperature for 3 h. The reaction was quenched by adding aqueous solution (30 mL), extracted with dichloromethane (50 mL×1) and allowed to stand for phase separation. The aqueous phase was washed with dichloromethane (100 mL×2). The combined organic phase was dried over anhydrous sodium sulfate and concentrated under reduced pressure to obtain a crude product from the residue, and the crude product was separated by preparative high-performance liquid chromatography to obtain compound 145 as a light yellow solid (50 mg, 50%). Preparative separation conditions: instrument: waters 2767 (preparative liquid phase chromatographic instrument); chromatographic column: XBridge@ Prep C18 (19 mm×250 mm); The sample was dissolved in DMF and filtered with a 0.45 m filter to prepare a sample solution. Preparative chromatography conditions: mobile phase A: acetonitrile; mobile phase B: water (containing 0.5% ammonia water); gradient elution: the content of mobile phase A rises from 45% to 75%; flow rate: 15 ml/min; elution time: 20 min; retention time: about 15.67 min;

¹H NMR (400 MHz, CDCl₃) δ 8.61 (s, 1H), 8.03 (s, 1H), 7.78-7.71 (m, 3H), 7.63 (s, 1H), 7.63 (s, 1H), 7.55-7.47 (m, 4H), 7.41-7.31 (m, 3H), 5.54-5.51 (m, 1H), 3.91 (s, 3H), 3.17-3.10 (m, 2H), 2.89-2.81 (m, 2H), 2.55-2.50 (m, 3H), 1.30-1.26 (m, 5H).

LC-MS (ESI): m/z=559.3 [M+H]⁺.

Compound 146:

### (R/S)-(4-((2-(difluoromethyl)-2H-tetrazol-5-yl)(phenyl)methyl)piperazin-1-yl)(4-(5-(1-isopropyl-1H-pyrazol-4-yl)benzo[d]oxazol-2-yl)pyridin-2-yl)methanone

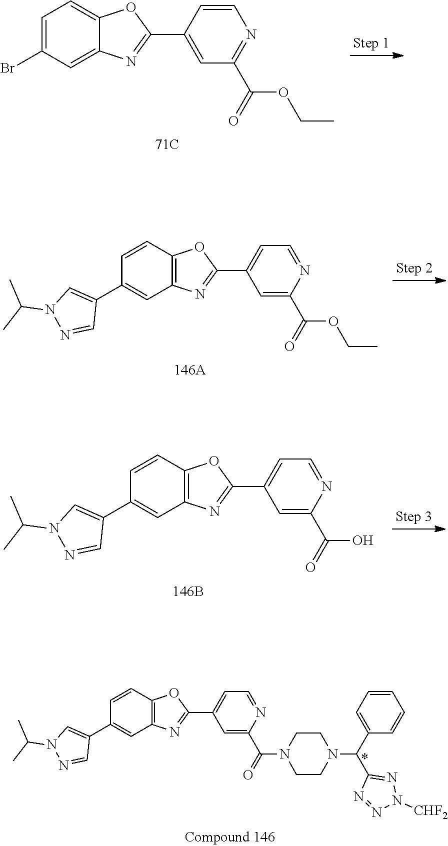

Step 1:

### Ethyl 4-(5-(1-isopropyl-1H-pyrazol-4-yl)benzo[d]oxazol-2-yl)picolinate (146A)

Compound 71C (260 mg, 0.75 mmol) was added to a mixed solvent of dioxane (10 mL) and water (1 mL), and then 1-isopropyl-4-(4,4,5,5-tetramethyl-1,3,2-dioxin-2-yl)-1H pyrazole (354 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 146A (180 mg, 64%).

LC-MS (ESI): m/z=377.2[M+H]⁺.

Step 2:

### 4-(5-(1-isopropyl-1H-pyrazol-4-yl)benzo[d]oxazol-2-yl)picolinic Acid (146B)

Anhydrous methanol (10 mL) and NaOH (96 mg, 2.39 mmol, 2 mL) aqueous solution were successively added to compound 146A (180 mg, 0.48 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 146B (100 mg, 60%).

LC-MS (ESI): m/z=349.1 [M+H]⁺.

Step 3:

### (R/S)-(4-((2-(difluoromethyl)-2H-tetrazol-5-yl)(phenyl)methyl)piperazin-1-yl)(4-(5-(1-isopropyl-1H-pyrazol-4-yl)benzo[d]oxazol-2-yl)pyridin-2-yl)methanone (Compound 146)

At room temperature, DMF (8 mL), intermediate 7 (85 mg, 0.29 mmol), HATU (165 mg, 0.44 mmol) and DIPEA (112 mg, 0.87 mmol) were successively added to compound 146B (100 mg, 0.29 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 146 (25 mg, 14%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.79 min.

LC-MS (ESI): m/z=625.3 [M+H]⁺.

¹H NMR (400 MHz, DMSO-d₆) δ8.81-8.82 (m, 1H), 8.55 (t, 1H), 8.33 (s, 1H), 8.18-8.19 (m, 1H), 8.14-8.15 (m, 1H), 8.10 (d, 1H), 7.97-7.98 (m, 1H), 7.83 (d, 1H), 7.74-7.77 (m, 1H), 7.49-7.50 (m, 2H), 7.37-7.41 (m, 2H), 7.31-7.35 (m, 1H), 5.32 (s, 1H), 4.49-4.55 (m, 1H), 3.72-3.73 (m, 2H), 3.50-3.51 (m, 2H), 2.58-2.61 (m, 1H), 2.47-2.49 (m, 2H), 2.33-2.37 (m, 1H), 1.47 (d, 6H).

Compound 147:

### (R/S)-(4-((2-(difluoromethyl)-2H-tetrazol-5-yl)(phenyl)methyl)piperazin-1-yl)(4-(5-(1-methyl-1H-imidazol-4-yl)benzo[d]oxazol-2-yl)pyridin-2-yl)methanone

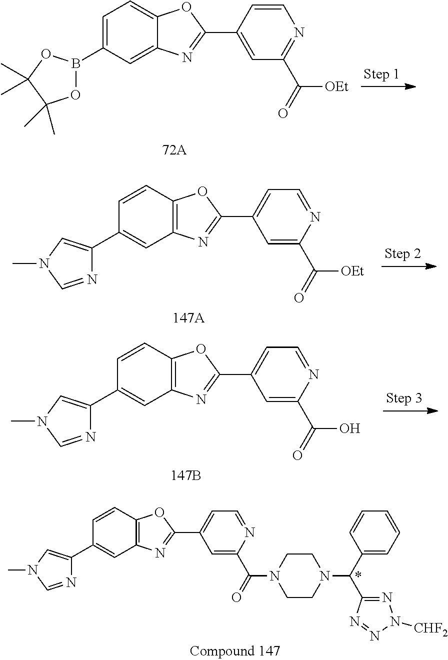 [SMILES: CCOC(=O)c1cc(-c2nc3cc(B4OC(C)(C)C(C)(C)O4)ccc3o2)ccn1 ; CCOC(=O)c1cc(-c2nc3cc(-c4cn(C)cn4)ccc3o2)ccn1 ; Cn1cnc(-c2ccc3oc(-c4ccnc(C(=O)O)c4)nc3c2)c1 ; Cn1cnc(-c2ccc3oc(-c4ccnc(C(=O)N5CCN(C(c6ccccc6)c6nnn(C(F)F)n6)CC5)c4)nc3c2)c1]

Step 1:

### Ethyl 4-(5-(1-methyl-1H-imidazol-4-yl)benzo[d]oxazol-2-yl)picolinate (147A)

Compound 72A (270 mg, 0.69 mmol) was added to a mixed solvent of dioxane (10 mL) and water (0.5 mL), and then 4-bromo-1-methyl-1H-imidazole (133 mg, 0.83 mmol), potassium carbonate (190 mg, 1.38 mmol) and Pd(dppf)Cl₂ (15 mg, 0.02 mmol) were added. The mixture was warmed to 100° C. and stirred for 3 h under nitrogen protection. The reaction was cooled to room temperature, and the filtrate was concentrated and then separated and purified by silica gel column chromatography (PE:EA=1:4) to obtain 147A (90 mg, 38%).

LC-MS (ESI): m/z=349.1[M+H]⁺.

Step 2:

### 4-(5-(1-methyl-1H-imidazol-4-yl)benzo[d]oxazol-2-yl)picolinic Acid (147B)

Anhydrous methanol (8 mL) and NaOH (52 mg, 1.29 mmol, 0.5 mL) aqueous solution were successively added to compound 147A (90 mg, 0.26 mmol), and the mixture was stirred at room temperature overnight. The reaction solution was adjusted to pH=2-3 by dropwise adding 1 N hydrochloric acid, concentrated under reduced pressure to remove the solvent and dried to obtain a crude product of compound 147B, which was directly used in the next reaction (130 mg).

LC-MS (ESI): m/z=321.1 [M+H]⁺.

Step 3:

### (R/S)-(4-((2-(difluoromethyl)-2H-tetrazol-5-yl)(phenyl)methyl)piperazin-1-yl)(4-(5-(1-methyl-1H-imidazol-4-yl)benzo[d]oxazol-2-yl)pyridin-2-yl)methanone

At room temperature, DMF (4 mL), intermediate 7 (59 mg, 0.20 mmol), HATU (114 mg, 0.3 mmol) and DIPEA (77 mg, 0.6 mmol) were successively added to compound 147B (65 mg, 0.20 mmol). The reaction was stirred for 1 hour. The reaction solution was poured into water (20 mL) and extracted with ethyl acetate (20 mL×3). The organic phase was concentrated, and the residue was separated and purified by HPLC to obtain compound 147 (10 mg, 8%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.86 min.

LC-MS (ESI): m/z=597.3 [M+H]⁺.

¹H NMR (400 MHz, DMSO-d₆) δ 9.01 (s, 1H), 8.83-8.84 (m, 1H), 8.56 (t, 1H), 8.29-8.30 (m, 1H), 8.22-8.23 (m, 1H), 8.17-8.19 (m, 2H), 8.01-8.04 (m, 1H), 7.90-7.92 (m, 1H), 7.49-7.51 (m, 2H), 7.38-7.41 (m, 2H), 7.32-7.35 (m, 1H), 5.34 (s, 1H), 3.90 (s, 3H), 3.72-3.73 (m, 2H), 3.52-3.53 (m, 2H), 2.60-2.63 (m, 1H), 2.47-2.50 (m, 2H), 2.36-2.39 (m, 1H).

Compound 148:

### (R/S)-(4-(5-(1-cyclopropyl-1H-pyrazol-4-yl)benzo[d]oxazol-2-yl)pyridin-2-yl)(4-((2-(difluoromethyl)-2H-tetrazol-5-yl)(phenyl)methyl)piperazin-1-yl)methanone

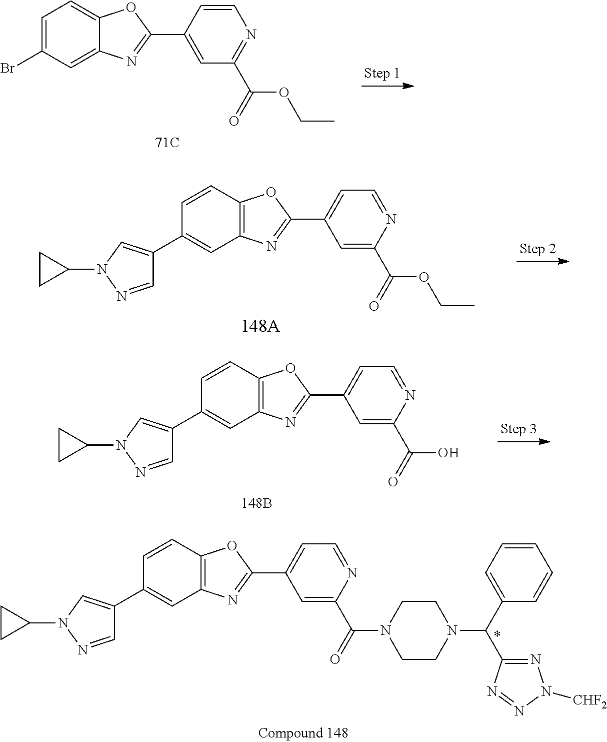 [SMILES: CCOC(=O)c1cc(-c2nc3cc(Br)ccc3o2)ccn1 ; CCOC(=O)c1cc(-c2nc3cc(-c4cnn(C5CC5)c4)ccc3o2)ccn1 ; O=C(O)c1cc(-c2nc3cc(-c4cnn(C5CC5)c4)ccc3o2)ccn1 ; O=C(c1cc(-c2nc3cc(-c4cnn(C5CC5)c4)ccc3o2)ccn1)N1CCN(C(c2ccccc2)c2nnn(C(F)F)n2)CC1]

Step 1:

### Ethyl 4-(5-(1-cyclopropyl-1H-pyrazol-4-yl)benzo[d]oxazol-2-yl)picolinate (148A)

Compound 71C (260 mg, 0.75 mmol) was added to a mixed solvent of dioxane (10 mL) and water (1 mL), and then 1-cyclopropyl-4-(4,4,5,5-tetramethyl-1,3,2-dioxin-2-yl)-1H pyrazole (351 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 148A (220 mg, 78%).

LC-MS (ESI): m/z=375.1[M+H]⁺.

Step 2:

### 4-(5-(1-cyclopropyl-1H-pyrazol-4-yl)benzo[d]oxazol-2-yl)picolinic Acid (148B)

Anhydrous methanol (10 mL) and NaOH (118 mg, 2.94 mmol, 2 mL) aqueous solution were successively added to compound 148A (220 mg, 0.59 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 148B (180 mg, 88%).

LC-MS (ESI): m/z=347.1 [M+H]⁺.

Step 3:

### (R/S)-(4-(5-(1-cyclopropyl-1H-pyrazol-4-yl)benzo[d]oxazol-2-yl)pyridin-2-yl)(4-((2-(difluoromethyl)-2H-tetrazol-5-yl)(phenyl)methyl)piperazin-1-yl)methanone

At room temperature, DMF (8 mL), intermediate 7 (85 mg, 0.29 mmol), HATU (165 mg, 0.44 mmol) and DIPEA (112 mg, 0.87 mmol) were successively added to compound 148B (100 mg, 0.29 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 148 (25 mg, 14%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.99 min.

LC-MS (ESI): m/z=623.3 [M+H]⁺.

¹H NMR (400 MHz, DMSO-d₆) δ8.80-8.82 (m, 1H), 8.55 (t, 1H), 8.34 (s, 1H), 8.18-8.19 (m, 1H), 8.14-8.15 (m, 1H), 8.10 (d, 1H), 7.97-7.98 (m, 1H), 7.82 (d, 1H), 7.74-7.76 (m, 1H), 7.49-7.50 (m, 2H), 7.37-7.41 (m, 2H), 7.31-7.35 (m, 1H), 5.32 (s, 1H), 3.75-3.78 (m, 1H), 3.71-3.74 (m, 2H), 3.50-3.51 (m, 2H), 2.58-2.61 (m, 1H), 2.47-2.49 (m, 2H), 2.33-2.37 (m, 1H), 1.08-1.12 (m, 2H), 0.97-1.04 (m, 2H).

Compound 149:

### (1S,2S)-2-fluoro-N-(2-(2-(4-((R/S)-(2-(fluoromethyl)-2H-tetrazol-5-yl)(phenyl)methyl)piperazine-1-carbonyl)pyridin-4-yl)benzo[d]oxazol-5-yl)cyclopropane-1-carboxamide

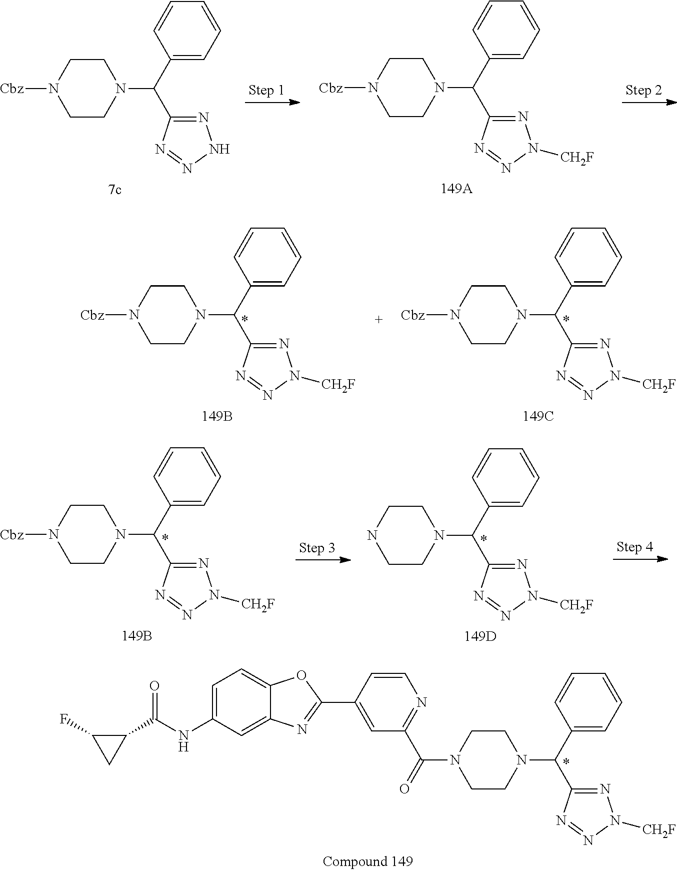

Step 1:

### Benzyl4-((2-(fluoromethyl)-2H-tetrazol-5-yl)(phenyl)methyl)piperazine-1-carboxylate (149A)

Compound 7c (18 g, 47.6 mmol) was added to DMF (300 mL), and NaH (60% in oil, 2.86 g, 71.4 mmol) was slowly added under an ice bath. The mixture was stirred for 1 hour; then fluoroiodomethane (9.14 g, 57.12 mmol) was added; and the resulting mixture was stirred at room temperature for another 2 hours. The reaction solution was poured into water (400 mL) to quench the reaction, extracted with ethyl acetate (200 mL×3), washed with saturated brine, dried over anhydrous sodium sulfate and then filtered. The organic phase was concentrated, and the residue was separated and purified by silica gel column chromatography (PE:EA=1:2) to obtain 149A (5.7 g, 29%).

LC-MS (ESI): m/z=411.2[M+H]⁺.

Step 2:

### Benzyl(R/S)-4-((2-(fluoromethyl)-2H-tetrazol-5-yl)(phenyl)methyl)piperazine-1-carboxylate (149B)

Compound 149A (1.7 g, 4.14 mmol) was purified and separated by chiral HPLC (chromatographic column: AD-H column, 250×30 mm I.D., 5 μm; mobile phase: A for n-hexane (0.1% DEA) and B for ethanol; gradient: B 40%; flow rate: 1 mL/min; column pressure: 100 bar; column temperature: 35° C.; absorption wavelength: 220 nm; cycle time: about 40 min) resolution to obtain 149B (retention time: 23.331 min, 600 mg, 1.46 mmol) and 149C (retention time: 25.623 min, 500 mg, 1.22 mmol).

Step 3:

### (R/S)-1-((2-(fluoromethyl)-2H-tetrazol-5-yl)(phenyl)methyl)piperazine (149D)

Compound 149B (600 mg, 1.46 mmol) was added to methanol (50 mL); then palladium on carbon (0.2 g) was added; and the mixture was stirred at 25° C. for 1 hours under the protection of hydrogen replacement. The reaction was filtered, and the filtrate was concentrated to obtain 149D as a colorless viscous liquid (300 mg, 74%).

LC-MS (ESI): m/z=277.1 [M+H]⁺.

Step 4:

### (1S,2S)-2-fluoro-N-(2-(2-(4-((R/S)-(2-(fluoromethyl)-2H-tetrazol-5-yl)(phenyl)methyl)piperazine-1-carbonyl)pyridin-4-yl)benzo[d]oxazol-5-yl)cyclopropane-1-carboxamide

At room temperature, DMF (8 mL), compound 117F (124 mg, 0.36 mmol), HATU (205 mg, 0.54 mmol) and DIPEA (139 mg, 1.08 mmol) were successively added to compound 149D (100 mg, 0.36 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 149 (25 mg, 12%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.06 min.

LC-MS (ESI): m/z=600.3 [M+H]⁺.

¹H NMR (400 MHz, DMSO-d₆) δ 10.47 (s, 1H), 8.79-8.81 (m, 1H), 8.24 (d, 1H), 8.17-8.18 (m, 1H), 8.13-8.14 (m, 1H), 7.80 (d, 1H), 7.61-7.63 (m, 1H), 7.48-7.50 (m, 2H), 7.36-7.40 (m, 2H), 7.30-7.33 (m, 1H), 6.79 (s, 1H), 6.66 (s, 1H), 5.24 (s, 1H), 5.01-5.05 (m, 0.5H), 4.85-4.87 (m, 0.5H), 3.71-3.72 (m, 2H), 3.49-3.50 (m, 2H), 2.57-2.60 (m, 1H), 2.47-2.49 (m, 2H), 2.32-2.35 (m, 1H), 2.00-2.06 (m, 1H), 1.62-1.72 (m, 1H), 1.14-1.21 (m, 1H).

Compound 150:

### (1S,2S)-2-fluoro-N-(2-(2-(4-((S/R)-(2-(fluoromethyl)-2H-tetrazol-5-yl)(phenyl)methyl)piperazine-1-carbonyl)pyridin-4-yl)benzo[d]oxazol-5-yl)cyclopropane-1-carboxamide

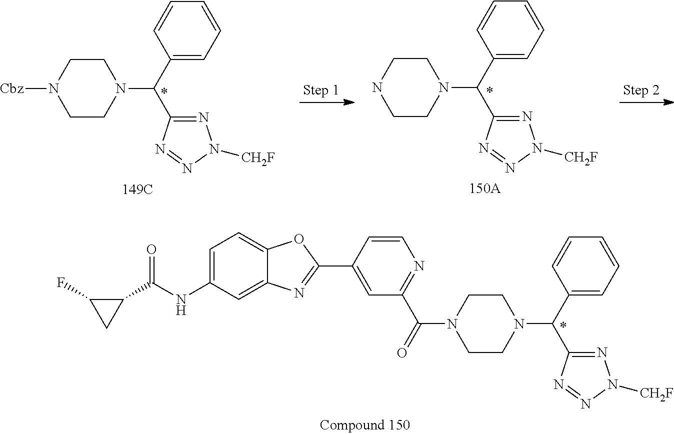

Step 1:

### (S/R)-1-((2-(fluoromethyl)-2H-tetrazol-5-yl)(phenyl)methyl)piperazine (150A)

Compound 149C (600 mg, 1.46 mmol) was added to methanol (50 mL); then palladium on carbon (0.2 g) was added; and the mixture was stirred at 25° C. for 1 hours under the protection of hydrogen replacement. The reaction was filtered, and the filtrate was concentrated to obtain 150A as a colorless viscous liquid (300 mg, 74%).

LC-MS (ESI): m/z=277.1 [M+H]⁺.

Step 2:

### (1S,2S)-2-fluoro-N-(2-(2-(4-((S)-(2-(fluoromethyl)-2H-tetrazol-5-yl)(phenyl)methyl)piperazine-1-carbonyl)pyridin-4-yl)benzo[d]oxazol-5-yl)cyclopropane-1-carboxamide (compound 150)

At room temperature, DMF (8 mL), compound 117F (124 mg, 0.36 mmol), HATU (205 mg, 0.54 mmol) and DIPEA (139 mg, 1.08 mmol) were successively added to compound 150A (100 mg, 0.36 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 150 (25 mg, 12%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.56 min.

LC-MS (ESI): m/z=600.3 [M+H]⁺.

¹H NMR (400 MHz, DMSO-d₆) δ 10.47 (s, 1H), 8.79-8.81 (m, 1H), 8.24 (d, 1H), 8.17-8.18 (m, 1H), 8.13-8.14 (m, 1H), 7.80 (d, 1H), 7.61-7.63 (m, 1H), 7.48-7.50 (m, 2H), 7.36-7.40 (m, 2H), 7.30-7.33 (m, 1H), 6.79 (s, 1H), 6.66 (s, 1H), 5.24 (s, 1H), 5.01-5.05 (m, 0.5H), 4.85-4.87 (m, 0.5H), 3.71-3.72 (m, 2H), 3.49-3.50 (m, 2H), 2.57-2.60 (m, 1H), 2.47-2.49 (m, 2H), 2.32-2.35 (m, 1H), 2.00-2.06 (m, 1H), 1.62-1.72 (m, 1H), 1.14-1.21 (m, 1H).

### Example 151

### (R/S)-(4-((2-(difluoromethyl)-2H-tetrazol-5-yl)(phenyl)methyl)piperazin-1-yl)(4-(5-(1-(oxetan-3-yl)-1H-pyrazol-4-yl)benzo[d]oxazol-2-yl)pyridin-2-yl)methanone

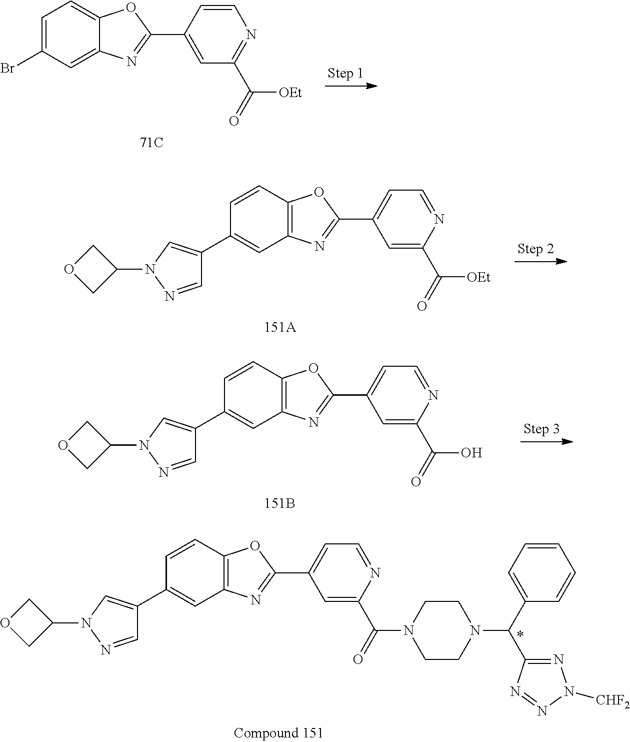 [SMILES: CCOC(=O)c1cc(-c2nc3cc(Br)ccc3o2)ccn1 ; CCOC(=O)c1cc(-c2nc3cc(-c4cnn(C5COC5)c4)ccc3o2)ccn1 ; O=C(O)c1cc(-c2nc3cc(-c4cnn(C5COC5)c4)ccc3o2)ccn1 ; O=C(c1cc(-c2nc3cc(-c4cnn(C5COC5)c4)ccc3o2)ccn1)N1CCN(C(c2ccccc2)c2nnn(C(F)F)n2)CC1]

Step 1:

### Ethyl 4-(5-(1-(oxetan-3-yl)-1H-pyrazol-4-yl)benzo[d]oxazol-2-yl)picolinate (151A)

Compound 71C (260 mg, 0.75 mmol) was added to a mixed solvent of dioxane (10 mL) and water (1 mL); 1-(oxetan-3-yl)-4-(4,4,5,5-tetramethyl-1,3,2-dioxaborolan-2-yl)-1H-pyrazole (375 mg, 1.50 mmol), potassium carbonate (207 mg, 1.50 mmol) and Pd(dppf)Cl₂ (58 mg, 0.08 mmol) were added; and the mixture was warmed to 100° C. and stirred for 7 h under nitrogen protection. The reaction was cooled to room temperature, and the filtrate was concentrated and then separated and purified by silica gel column chromatography (PE:EA=1:4) to obtain 151A (200 mg, 68%).

LC-MS (ESI): m/z=391.1[M+H]⁺.

Step 2:

### 4-(5-(1-(oxetan-3-yl)-1H-pyrazol-4-yl)benzo[d]oxazol-2-yl)picolinic Acid (151B)

Anhydrous methanol (10 mL) and NaOH (77 mg, 1.92 mmol, 2 mL) aqueous solution were successively added to compound 151A (150 mg, 0.38 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 151B (130 mg, 94%).

LC-MS (ESI): m/z=363.1 [M+H]⁺.

Step 3:

### (R/S)-(4-((2-(difluoromethyl)-2H-tetrazol-5-yl)(phenyl)methyl)piperazin-1-yl)(4-(5-(1-(oxetan-3-yl)-1H-pyrazol-4-yl)benzo[d]oxazol-2-yl)pyridin-2-yl)methanone (Compound 151)

At room temperature, DMF (8 mL), intermediate 7 (105 mg, 0.36 mmol), HATU (205 mg, 0.54 mmol) and DIPEA (139 mg, 1.08 mmol) were successively added to compound 151B (130 mg, 0.36 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 151 (40 mg, 17%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.19 min.

LC-MS (ESI): m/z=639.3 [M+H]⁺.

¹H NMR (400 MHz, DMSO-d₆) δ8.81-8.82 (m, 1H), 8.56 (t, 1H), 8.48 (s, 1H), 8.18-8.19 (m, 1H), 8.13-8.16 (m, 3H), 7.85 (d, 1H), 7.76-7.79 (m, 1H), 7.49-7.50 (m, 2H), 7.37-7.41 (m, 2H), 7.31-7.35 (m, 1H), 5.57-5.64 (m, 1H), 5.32 (s, 1H), 4.93-4.99 (m, 4H), 3.72-3.73 (m, 2H), 3.50-3.51 (m, 2H), 2.58-2.61 (m, 1H), 2.47-2.49 (m, 2H), 2.33-2.37 (m, 1H).

### Example 152

### (R/S)-(4-((2-(difluoromethyl)-2H-tetrazol-5-yl)(phenyl)methyl)piperazin-1-yl)(4-(5-(1-methyl-1H-pyrazol-5-yl)benzo[d]oxazol-2-yl)pyridin-2-yl)methanone

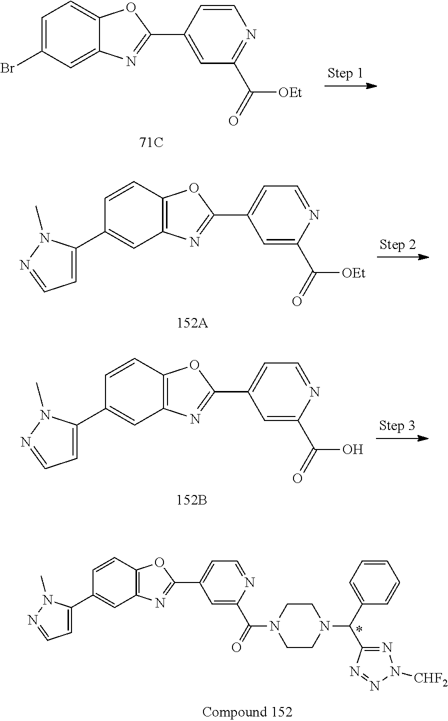 [SMILES: CCOC(=O)c1cc(-c2nc3cc(Br)ccc3o2)ccn1 ; CCOC(=O)c1cc(-c2nc3cc(-c4ccnn4C)ccc3o2)ccn1 ; Cn1nccc1-c1ccc2oc(-c3ccnc(C(=O)O)c3)nc2c1 ; Cn1nccc1-c1ccc2oc(-c3ccnc(C(=O)N4CCN(C(c5ccccc5)c5nnn(C(F)F)n5)CC4)c3)nc2c1]

Step 1:

### Ethyl 4-(5-(1-methyl-1H-pyrazol-5-yl)benzo[d]oxazol-2-yl)picolinate (152A)

Compound 71c (260 mg, 0.75 mmol) was added to a mixed solvent of dioxane (10 mL) and water (1 mL), and then 1-methyl-5-(4,4,5,5-tetramethyl-1,3,2-dioxin-2-yl)-1H pyrazole (312 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 152A (150 mg, 57%).

LC-MS (ESI): m/z=349.1[M+H]⁺.

Step 2:

### 4-(5-(1-methyl-1H-pyrazol-5-yl)benzo[d]oxazol-2-yl)picolinic Acid (152B)

Anhydrous methanol (10 mL) and NaOH (86 mg, 2.16 mmol, 2 mL) aqueous solution were successively added to compound 152A (150 mg, 0.43 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 152B (130 mg, 94%).

LC-MS (ESI): m/z=321.1 [M+H]⁺.

Step 3:

### (R/S)-(4-((2-(difluoromethyl)-2H-tetrazol-5-yl)(phenyl)methyl)piperazin-1-yl)(4-(5-(1-methyl-1H-pyrazol-5-yl)benzo[d]oxazol-2-yl)pyridin-2-yl)methanone (compound 152)

At room temperature, DMF (8 mL), intermediate 7 (119 mg, 0.41 mmol), HATU (233 mg, 0.62 mmol) and DIPEA (159 mg, 1.23 mmol) were successively added to compound 152B (130 mg, 0.41 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 152 (45 mg, 18%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.43 min.

LC-MS (ESI): m/z=597.3 [M+H]⁺.

¹H NMR (400 MHz, DMSO-d₆) δ8.84 (d, 1H), 8.56 (t, 1H), 8.22-8.23 (m, 1H), 8.17-8.19 (m, 1H), 8.07-8.08 (m, 1H), 7.98 (d, 1H), 7.66-7.68 (m, 1H), 7.49-7.51 (m, 3H), 7.37-7.41 (m, 2H), 7.31-7.35 (m, 1H), 6.49 (d, 1H), 5.32 (s, 1H), 3.90 (s, 3H), 3.72-3.73 (m, 2H), 3.51-3.52 (m, 2H), 2.58-2.61 (m, 1H), 2.47-2.49 (m, 2H), 2.33-2.37 (m, 1H).

### Example 153

### (R/S)-(4-((2-(difluoromethyl)-2H-tetrazol-5-yl)(phenyl)methyl)piperazin-1-yl)(4-(5-(1-methyl-1H-pyrazol-3-yl)benzo[d]oxazol-2-yl)pyridin-2-yl)methanone

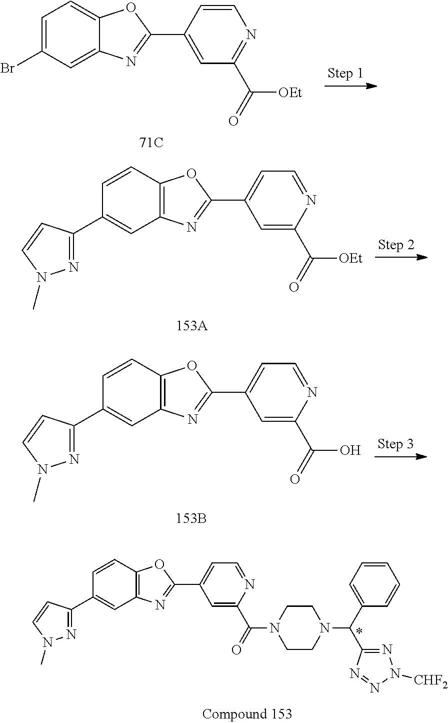 [SMILES: CCOC(=O)c1cc(-c2nc3cc(Br)ccc3o2)ccn1 ; CCOC(=O)c1cc(-c2nc3cc(-c4ccn(C)n4)ccc3o2)ccn1 ; Cn1ccc(-c2ccc3oc(-c4ccnc(C(=O)O)c4)nc3c2)n1 ; Cn1ccc(-c2ccc3oc(-c4ccnc(C(=O)N5CCN(C(c6ccccc6)c6nnn(C(F)F)n6)CC5)c4)nc3c2)n1]

Step 1:

### Ethyl 4-(5-(1-methyl-1H-pyrazol-3-yl)benzo[d]oxazol-2-yl)picolinate (153A)

Compound 71C (260 mg, 0.75 mmol) was added to a mixed solvent of dioxane (10 mL) and water (1 mL), and then 1-methyl-3-(4,4,5,5-tetramethyl-1,3,2-dioxin-2-yl)-1H pyrazole (312 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 153A (150 mg, 57%).

LC-MS (ESI): m/z=349.1[M+H]⁺.

Step 2:

### 4-(5-(1-methyl-1H-pyrazol-3-yl)benzo[d]oxazol-2-yl)picolinic Acid (153B)

Anhydrous methanol (10 mL) and NaOH (86 mg, 2.16 mmol, 2 mL) aqueous solution were successively added to compound 153A (150 mg, 0.43 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 153B (120 mg, 87%).

LC-MS (ESI): m/z=321.1 [M+H]⁺.

Step 3:

### (R/S)-(4-((2-(difluoromethyl)-2H-tetrazol-5-yl)(phenyl)methyl)piperazin-1-yl)(4-(5-(1-methyl-1H-pyrazol-3-yl)benzo[d]oxazol-2-yl)pyridin-2-yl)methanone (Compound 153)

At room temperature, DMF (8 mL), intermediate 7 (119 mg, 0.41 mmol), HATU (233 mg, 0.62 mmol) and DIPEA (159 mg, 1.23 mmol) were successively added to compound 153B (130 mg, 0.41 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 153 (40 mg, 16%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.86 min.

LC-MS (ESI): m/z=597.3 [M+H]⁺.

¹H NMR (400 MHz, DMSO-d₆) δ8.81-8.82 (m, 1H), 8.56 (t, 1H), 8.22-8.23 (m, 1H), 8.20-8.21 (m, 1H), 8.15-8.17 (m, 1H), 7.97-8.00 (m, 1H), 7.84-7.87 (m, 1H), 7.77 (d, 1H), 7.49-7.50 (m, 2H), 7.37-7.41 (m, 2H), 7.31-7.35 (m, 1H), 6.82 (d, 1H), 5.32 (s, 1H), 3.91 (s, 3H), 3.72-3.73 (m, 2H), 3.51-3.52 (m, 2H), 2.58-2.61 (m, 1H), 2.47-2.49 (m, 2H), 2.33-2.37 (m, 1H).

### Example 154

### (R/S)-(4-((2-(difluoromethyl)-2H-tetrazol-5-yl)(phenyl)methyl)piperazin-1-yl)(4-(5-(1,5-dimethyl-1H-pyrazol-4-yl)benzo[d]oxazol-2-yl)pyridin-2-yl)methanone

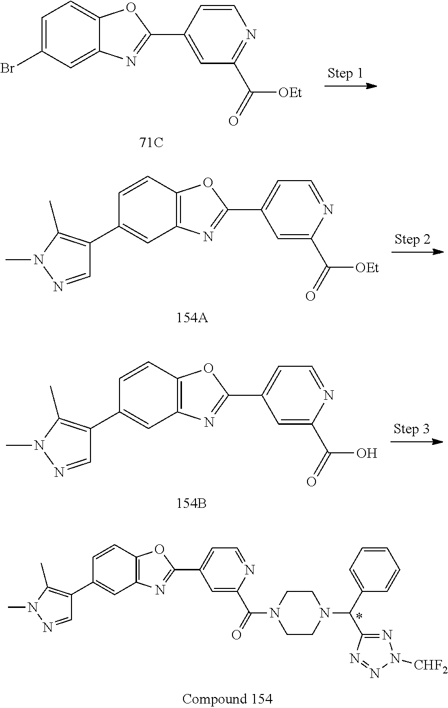 [SMILES: CCOC(=O)c1cc(-c2nc3cc(Br)ccc3o2)ccn1 ; CCOC(=O)c1cc(-c2nc3cc(-c4cnn(C)c4C)ccc3o2)ccn1 ; Cc1c(-c2ccc3oc(-c4ccnc(C(=O)O)c4)nc3c2)cnn1C ; Cc1c(-c2ccc3oc(-c4ccnc(C(=O)N5CCN(C(c6ccccc6)c6nnn(C(F)F)n6)CC5)c4)nc3c2)cnn1C]

Step 1:

### Ethyl 4-(5-(1,5-dimethyl-1H-pyrazol-4-yl)benzo[d]oxazol-2-yl)picolinate (154A)

Compound 71C (260 mg, 0.75 mmol) was added to a mixed solvent of dioxane (10 mL) and water (1 mL), and then 1,5-dimethyl-4-(4,4,5,5-tetramethyl-1,3,2-dioxin-2-yl)-1H pyrazole (333 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 154A (80 mg, 29%).

LC-MS (ESI): m/z=363.1[M+H]⁺.

Step 2:

### 4-(5-(1,5-dimethyl-1H-pyrazol-4-yl)benzo[d]oxazol-2-yl)picolinic Acid (154B)

Anhydrous methanol (7 mL) and NaOH (72 mg, 1.80 mmol, 2 mL) aqueous solution were successively added to compound 154A (130 mg, 0.36 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 154B (100 mg, 83%).

LC-MS (ESI): m/z=335.1 [M+H]⁺.

Step 3:

### (R/S)-(4-((2-(difluoromethyl)-2H-tetrazol-5-yl)(phenyl)methyl)piperazin-1-yl)(4-(5-(1,5-dimethyl-1H-pyrazol-4-yl)benzo[d]oxazol-2-yl)pyridin-2-yl)methanone (Compound 154)

At room temperature, DMF (8 mL), intermediate 7 (85 mg, 0.29 mmol), HATU (165 mg, 0.44 mmol) and DIPEA (112 mg, 0.87 mmol) were successively added to compound 154B (99 mg, 0.29 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 154 (30 mg, 17%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.02 min.

LC-MS (ESI): m/z=611.3 [M+H]⁺.

¹H NMR (400 MHz, DMSO-d₆) δ8.81-8.83 (m, 1H), 8.56 (t, 1H), 8.20-8.21 (m, 1H), 8.15-8.17 (m, 1H), 7.85-7.88 (m, 2H), 7.65 (s, 1H), 7.53-7.55 (m, 1H), 7.49-7.50 (m, 2H), 7.37-7.41 (m, 2H), 7.31-7.35 (m, 1H), 5.32 (s, 1H), 3.81 (s, 3H), 3.72-3.73 (m, 2H), 3.51-3.52 (m, 2H), 2.58-2.61 (m, 1H), 2.47-2.49 (m, 2H), 2.41 (s, 3H), 2.33-2.37 (m, 1H).

### Example 155

### (R/S)-(4-((2-(difluoromethyl)-2H-tetrazol-5-yl)(phenyl)methyl)piperazin-1-yl)(4-(5-(3-methyl-1H-pyrazol-4-yl)benzo[d]oxazol-2-yl)pyridin-2-yl)methanone

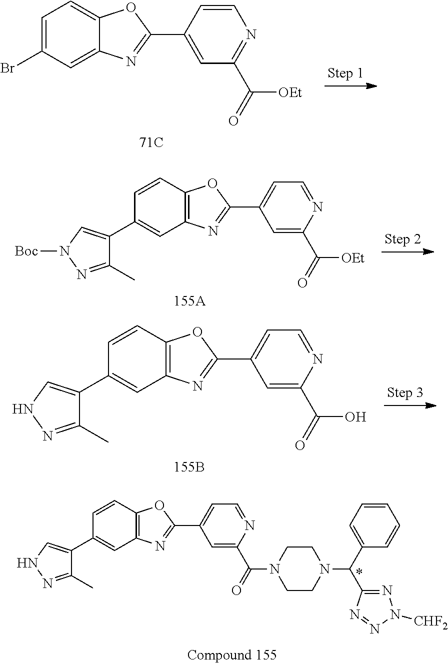 [SMILES: CCOC(=O)c1cc(-c2nc3cc(Br)ccc3o2)ccn1 ; CCOC(=O)c1cc(-c2nc3cc(-c4cn(C(=O)OC(C)(C)C)nc4C)ccc3o2)ccn1 ; Cc1n[nH]cc1-c1ccc2oc(-c3ccnc(C(=O)O)c3)nc2c1 ; Cc1n[nH]cc1-c1ccc2oc(-c3ccnc(C(=O)N4CCN(C(c5ccccc5)c5nnn(C(F)F)n5)CC4)c3)nc2c1]

Step 1:

### Ethyl 4-(5-(1-(tert-butoxycarbonyl)-3-methyl-1H-pyrazol-4-yl)benzo[d]oxazol-2-yl)picolinate (155A)

Compound 71C (260 mg, 0.75 mmol) was added to a mixed solvent of dioxane (10 mL) and water (1 mL); tert-butyl 3-methyl-4-(4,4,5,5-tetramethyl-1,3,2-dioxin-2-yl)-1H-pyrazole-1-carboxylate (333 mg, 1.50 mmol), potassium carbonate (207 mg, 1.50 mmol) and Pd(dppf)Cl₂ (58 mg, 0.08 mmol) were added; and the mixture was warmed to 100° C. and stirred for 3 h under nitrogen protection. The reaction was cooled to room temperature, and the filtrate was concentrated and then separated and purified by silica gel column chromatography (PE:EA=1:4) to obtain 155A (70 mg, 21%).

LC-MS (ESI): m/z=449.2[M+H]⁺.

Step 2:

### 4-(5-(3-methyl-1H-pyrazol-4-yl)benzo[d]oxazol-2-yl)picolinic Acid (155B)

Anhydrous methanol (7 mL) and NaOH (31 mg, 0.80 mmol, 2 mL) aqueous solution were successively added to compound 155A (70 mg, 0.16 mmol), and the mixture was stirred at 25° C. for 10 h. TLC or LCMS showed that the reaction of the raw materials was completed. The reaction solution was adjusted to pH=1-2 by dropwise adding 6 N hydrochloric acid, stirred for half an hour and concentrated under reduced pressure to remove the solvent. A small amount of water (2 mL) was added, and the mixture was stirred and then filtered. The filter cake was dried to obtain intermediate 155B (50 mg, 98%).

LC-MS (ESI): m/z=321.1 [M+H]⁺.

Step 3:

### (R/S)-(4-((2-(difluoromethyl)-2H-tetrazol-5-yl)(phenyl)methyl)piperazin-1-yl)(4-(5-(3-methyl-1H-pyrazol-4-yl)benzo[d]oxazol-2-yl)pyridin-2-yl)methanone (Compound 155)

At room temperature, DMF (8 mL), intermediate 7 (119 mg, 0.41 mmol), HATU (233 mg, 0.62 mmol) and DIPEA (159 mg, 1.23 mmol) were successively added to compound 155B (130 mg, 0.41 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 155 (40 mg, 16%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.78 min.

LC-MS (ESI): m/z=597.3 [M+H]⁺.

¹H NMR (400 MHz, DMSO-d₆) δ8.81-8.83 (m, 1H), 8.56 (t, 1H), 8.20-8.21 (m, 1H), 8.15-8.17 (m, 1H), 7.91-7.92 (m, 1H), 7.85-7.88 (m, 2H), 7.59-7.62 (m, 1H), 7.50-7.52 (m, 2H), 7.38-7.42 (m, 2H), 7.34-7.36 (m, 1H), 5.40 (s, 1H), 3.73-3.74 (m, 2H), 3.53-3.54 (m, 2H), 2.60-2.62 (m, 1H), 2.49-2.50 (m, 2H), 2.43-2.47 (m, 1H), 2.42 (s, 3H).

### Example 156

### (R/S)-(4-((2-(difluoromethyl)-2H-tetrazol-5-yl)(phenyl)methyl)piperazin-1-yl)(4-(5-(3,5-dimethyl-1H-pyrazol-4-yl)benzo[d]oxazol-2-yl)pyridin-2-yl)methanone

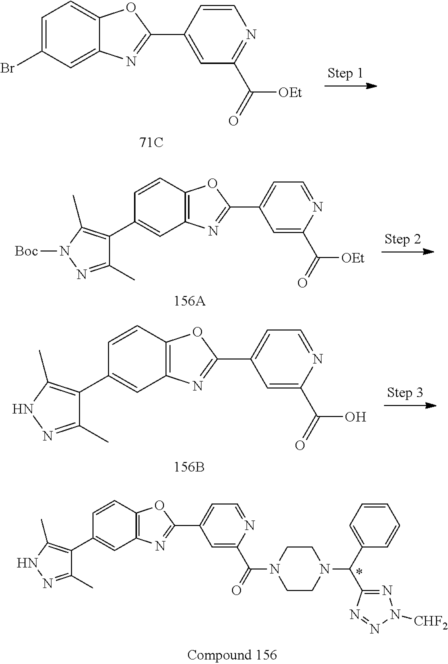 [SMILES: CCOC(=O)c1cc(-c2nc3cc(Br)ccc3o2)ccn1 ; CCOC(=O)c1cc(-c2nc3cc(-c4c(C)nn(C(=O)OC(C)(C)C)c4C)ccc3o2)ccn1 ; Cc1n[nH]c(C)c1-c1ccc2oc(-c3ccnc(C(=O)O)c3)nc2c1 ; Cc1n[nH]c(C)c1-c1ccc2oc(-c3ccnc(C(=O)N4CCN(C(c5ccccc5)c5nnn(C(F)F)n5)CC4)c3)nc2c1]

Step 1:

### Ethyl 4-(5-(1-(tert-butoxycarbonyl)-3,5-dimethyl-1H-pyrazol-4-yl)benzo[d]oxazol-2-yl)picolinate (156A)

Compound 71C (260 mg, 0.75 mmol) was added to a mixed solvent of dioxane (10 mL) and water (1 mL); tert-butyl 3,5-dimethyl-4-(4,4,5,5-tetramethyl-1,3,2-dioxin-2-yl)-1H-pyrazole-1-carboxylate (322 mg, 1.00 mmol), potassium carbonate (207 mg, 1.50 mmol) and Pd(dppf)Cl₂ (58 mg, 0.08 mmol) were added; and the mixture was warmed to 100° C. and stirred for 3 h under nitrogen protection. The reaction was cooled to room temperature, and the filtrate was concentrated and then separated and purified by silica gel column chromatography (PE:EA=1:4) to obtain 156A (130 mg, 38%).

LC-MS (ESI): m/z=463.2[M+H]⁺.

Step 2:

### 4-(5-(3,5-dimethyl-1H-pyrazol-4-yl)benzo[d]oxazol-2-yl)picolinic Acid (156B)

Anhydrous methanol (7 mL) and NaOH (56 mg, 1.40 mmol, 2 mL) aqueous solution were successively added to compound 156A (130 mg, 0.28 mmol), and the mixture was stirred at 25° C. for 10 h. TLC or LCMS showed that the reaction of the raw materials was completed. The reaction solution was adjusted to pH=1-2 by dropwise adding 6 N hydrochloric acid, stirred for half an hour and concentrated under reduced pressure to remove the solvent. A small amount of water (2 mL) was added, and the mixture was stirred and then filtered. The filter cake was dried to obtain intermediate 156B (85 mg, 90%).

LC-MS (ESI): m/z=335.1 [M+H]⁺.

Step 3:

### (R/S)-(4-((2-(difluoromethyl)-2H-tetrazol-5-yl)(phenyl)methyl)piperazin-1-yl)(4-(5-(3,5-dimethyl-1H-pyrazol-4-yl)benzo[d]oxazol-2-yl)pyridin-2-yl)methanone (Compound 156)

At room temperature, DMF (8 mL), intermediate 7 (123 mg, 0.29 mmol), HATU (239 mg, 0.63 mmol) and DIPEA (163 mg, 1.26 mmol) were successively added to compound 156B (140 mg, 0.42 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 156 (45 mg, 17%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.93 min.

LC-MS (ESI): m/z=611.3 [M+H]⁺.

¹H NMR (400 MHz, DMSO-d₆) δ 12.33 (s, 1H), 8.81-8.82 (m, 1H), 8.56 (t, 1H), 8.20-8.21 (m, 1H), 8.15-8.17 (m, 1H), 7.87 (d, 1H), 7.76-7.77 (m, 1H), 7.49-7.50 (m, 2H), 7.37-7.44 (m, 3H), 7.31-7.35 (m, 1H), 5.32 (s, 1H), 3.72-3.73 (m, 2H), 3.51-3.52 (m, 2H), 2.58-2.61 (m, 1H), 2.47-2.49 (m, 2H), 2.33-2.38 (m, 1H), 2.23 (s, 6H).

### Example 157

### (R/S)-3-(2-(2-(4-((2-(difluoromethyl)-2H-tetrazol-5-yl)(phenyl)methyl)piperazine-1-carbonyl)pyridin-4-yl)benzo[d]oxazol-5-yl)-1,2,4-oxadiazol-5(4H)-one

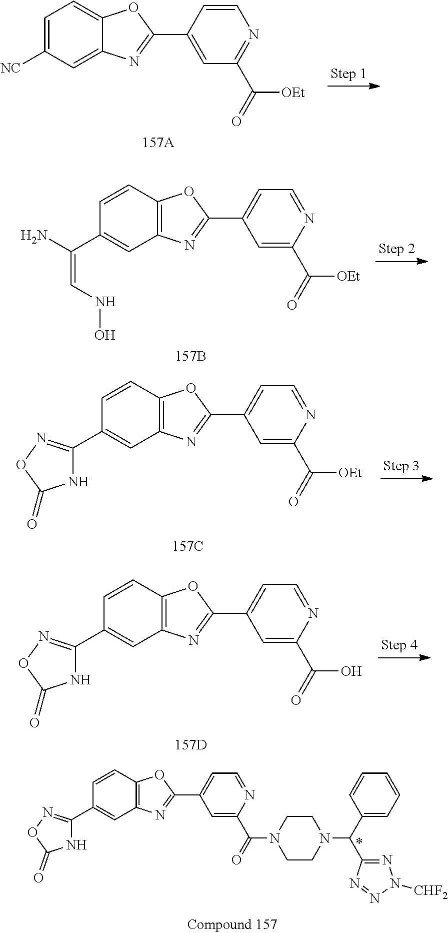 [SMILES: CCOC(=O)c1cc(-c2nc3cc(C#N)ccc3o2)ccn1 ; CCOC(=O)c1cc(-c2nc3cc(/C(N)=C\NO)ccc3o2)ccn1 ; CCOC(=O)c1cc(-c2nc3cc(-c4noc(=O)[nH]4)ccc3o2)ccn1 ; O=C(O)c1cc(-c2nc3cc(-c4noc(=O)[nH]4)ccc3o2)ccn1 ; O=C(c1cc(-c2nc3cc(-c4noc(=O)[nH]4)ccc3o2)ccn1)N1CCN(C(c2ccccc2)c2nnn(C(F)F)n2)CC1]

Step 1:

### Ethyl 4-(5-(1-amino-2-(hydroxyamino)vinyl)benzo[d]oxazol-2-yl)picolinate (157B)

Compound 157A (100 mg, 0.34 mmol) was added to anhydrous ethanol (5 mL) solvent, and then hydroxylamine hydrochloride (47 mg, 0.69 mmol) and DIPEA (132 mg, 1.02 mmol) were added. The mixture was warmed to 78° C. and stirred for 4 h. The reaction was cooled to room temperature, and the filtrate was concentrated and then separated and purified by silica gel column chromatography (PE:EA=1:1) to obtain 157B (90 mg, 78%).

LC-MS (ESI): m/z=341.1[M+H]⁺.

Step 2:

### Ethyl 4-(5-(5-oxo-4,5-dihydro-1,2,4-oxadiazol-3-yl)benzo[d]oxazol-2-yl)picolinate (157C)

Compound 157B (90 mg, 0.26 mmol) was added to dioxane (5 mL) solvent, and then CDI (64 mg, 0.40 mmol) and DBU (109 mg, 0.72 mmol) were added. The mixture was warmed to 100° C. and stirred for 4 h. The reaction was cooled to room temperature, and the filtrate was concentrated and then separated and purified by silica gel column chromatography (PE:EA=1:4) to obtain 157C (70 mg, 76%).

LC-MS (ESI): m/z=353.1[M+H]⁺.

Step 3:

### 4-(5-(5-oxo-4,5-dihydro-1,2,4-oxadiazol-3-yl)benzo[d]oxazol-2-yl)picolinic Acid (157D)

Anhydrous methanol (5 mL) and NaOH (40 mg, 1.00 mmol, 2 mL) aqueous solution were successively added to compound 157C (70 mg, 0.20 mmol), and the mixture was stirred at 25° C. for 10 h. TLC or LCMS showed that the reaction of the raw materials was completed. The reaction solution was adjusted to pH=5-6 by dropwise adding 2 N hydrochloric acid and concentrated under reduced pressure to remove the solvent. A small amount of water (2 mL) was added, and the mixture was stirred and then filtered. The filter cake was dried to obtain intermediate 157D (60 mg, 92%).

LC-MS (ESI): m/z=325.1 [M+H]⁺.

Step 4:

### (R/S)-3-(2-(2-(4-((2-(difluoromethyl)-2H-tetrazol-5-yl)(phenyl)methyl)piperazine-1-carbonyl)pyridin-4-yl)benzo[d]oxazol-5-yl)-1,2,4-oxadiazol-5(4H)-one

At room temperature, DMF (8 mL), intermediate 7 (118 mg, 0.40 mmol), HATU (228 mg, 0.60 mmol) and DIPEA (155 mg, 1.20 mmol) were successively added to compound 157D (130 mg, 0.40 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 157 (45 mg, 19%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.58 min.

LC-MS (ESI): m/z=601.3 [M+H]⁺.

¹H NMR (400 MHz, DMSO-d₆) δ8.82-8.84 (m, 1H), 8.55 (t, 1H), 8.27-8.28 (m, 1H), 8.22-8.23 (m, 1H), 8.18-8.19 (m, 1H), 7.96-8.03 (m, 2H), 7.48-7.50 (m, 2H), 7.37-7.41 (m, 2H), 7.31-7.35 (m, 1H), 5.32 (s, 1H), 3.72-3.73 (m, 2H), 3.50-3.51 (m, 2H), 2.58-2.61 (m, 1H), 2.47-2.49 (m, 2H), 2.33-2.37 (m, 1H).

### Example 158

### (R/S)-(4-((2-(difluoromethyl)-2H-tetrazol-5-yl)(phenyl)methyl)piperazin-1-yl)(4-(5-(4-methyl-1H-imidazol-1-yl)benzo[d]oxazol-2-yl)pyridin-2-yl)methanone

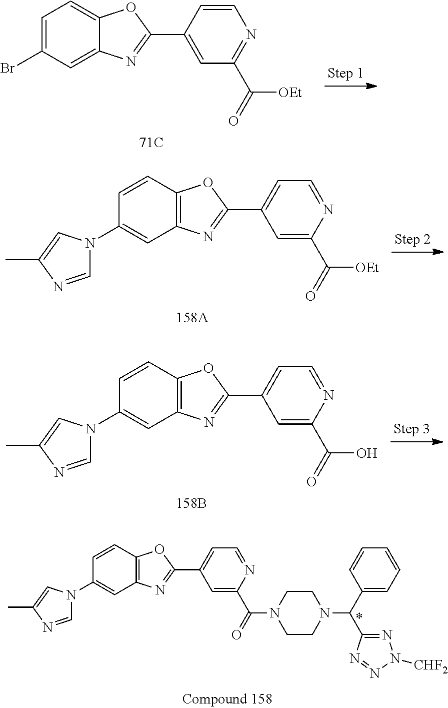 [SMILES: CCOC(=O)c1cc(-c2nc3cc(Br)ccc3o2)ccn1 ; CCOC(=O)c1cc(-c2nc3cc(-n4cnc(C)c4)ccc3o2)ccn1 ; Cc1cn(-c2ccc3oc(-c4ccnc(C(=O)O)c4)nc3c2)cn1 ; Cc1cn(-c2ccc3oc(-c4ccnc(C(=O)N5CCN(C(c6ccccc6)c6nnn(C(F)F)n6)CC5)c4)nc3c2)cn1]

Step 1:

### Ethyl 4-(5-(4-methyl-1H-imidazol-1-yl)benzo[d]oxazol-2-yl)picolinate (158A)

Compound 71C (400 mg, 1.15 mmol) and 4-methyl-1H-imidazole (189 mg, 2.30 mmol) were added to DMSO (5 mL) solvent, and then N1,N2-dimethylethane-1,2-diamine (202 mg, 2.30 mmol), cuprous iodide (44 mg, 0.23 mmol) and triethylamine (348 mg, 3.45 mmol) were added. The mixture was warmed to 120° C. and stirred for 6 h under nitrogen protection. The reaction was cooled to room temperature, diluted by adding water, extracted with EA, dried over anhydrous sodium sulfate and filtered. The filtrate was concentrated and then separated and purified by silica gel column chromatography (PE:EA=1:4) to obtain 158A (130 mg, 33%).

LC-MS (ESI): m/z=349.1[M+H]⁺.

Step 2:

### 4-(5-(4-methyl-1H-imidazol-1-yl)benzo[d]oxazol-2-yl)picolinic Acid (158B)

Anhydrous methanol (7 mL) and NaOH (60 mg, 1.49 mmol, 2 mL) aqueous solution were successively added to compound 158A (130 mg, 0.37 mmol), and the mixture was stirred at 25° C. for 4 h. TLC or LCMS showed that the reaction of the raw materials was completed. The reaction solution was adjusted to pH=1-2 by dropwise adding 6 N hydrochloric acid, stirred for half an hour and concentrated under reduced pressure to remove the solvent. A small amount of water (2 mL) was added, and the mixture was stirred and then filtered. The filter cake was dried to obtain intermediate 158B (100 mg, 85%).

LC-MS (ESI): m/z=321.1 [M+H]⁺.

Step 3:

### (R/S)-(4-((2-(difluoromethyl)-2H-tetrazol-5-yl)(phenyl)methyl)piperazin-1-yl)(4-(5-(4-methyl-1H-imidazol-1-yl)benzo[d]oxazol-2-yl)pyridin-2-yl)methanone (Compound 158)

At room temperature, DMF (8 mL), intermediate 7 (85 mg, 0.29 mmol), HATU (165 mg, 0.44 mmol) and DIPEA (112 mg, 0.87 mmol) were successively added to compound 158B (100 mg, 0.29 mmol). The reaction was stirred for 1 hour. The reaction solution was poured into water (30 mL) and extracted with ethyl acetate (20 mL×3). The organic phase was concentrated, and the residue was separated and purified by HPLC to obtain compound 158 (25 mg, 14%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.43 min.

LC-MS (ESI): m/z=597.3 [M+H]⁺.

¹H NMR (400 MHz, DMSO-d₆) δ8.82-8.84 (m, 1H), 8.56 (t, 1H), 8.20-8.21 (m, 2H), 8.16-8.17 (m, 2H), 7.980 (d, 1H), 7.75-7.78 (m, 1H), 7.53-7.546 (m, 1H), 7.49-7.50 (m, 2H), 7.37-7.41 (m, 2H), 7.31-7.35 (m, 1H), 5.32 (s, 1H), 3.71-3.73 (m, 2H), 3.50-3.51 (m, 2H), 2.58-2.61 (m, 1H), 2.47-2.49 (m, 2H), 2.33-2.37 (m, 1H), 2.19 (s, 3H).

### Example 159

### 4-(5-(1-methyl-1H-imidazol-5-yl)benzo[d]oxazol-2-yl)pyridin-2-yl)(4-((2-methyl-2H-tetrazol-5-yl)(phenyl)methyl)piperazin-1-yl)methanone

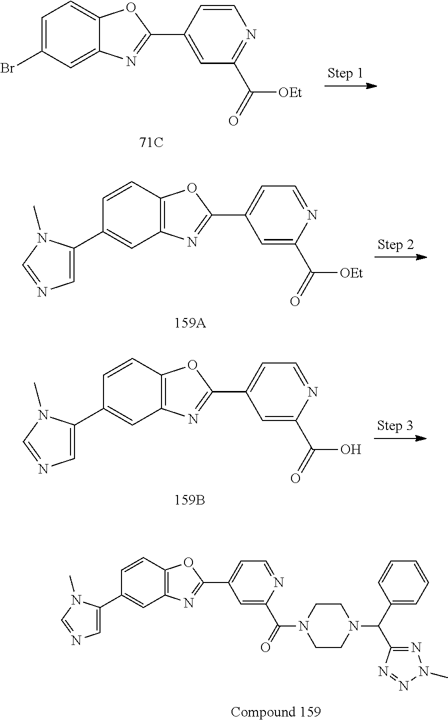 [SMILES: CCOC(=O)c1cc(-c2nc3cc(Br)ccc3o2)ccn1 ; CCOC(=O)c1cc(-c2nc3cc(-c4cncn4C)ccc3o2)ccn1 ; Cn1cncc1-c1ccc2oc(-c3ccnc(C(=O)O)c3)nc2c1 ; Cn1nnc(C(c2ccccc2)N2CCN(C(=O)c3cc(-c4nc5cc(-c6cncn6C)ccc5o4)ccn3)CC2)n1]

Step 1:

### Ethyl 4-(5-(1-methyl-1H-imidazol-5-yl)benzo[d]oxazol-2-yl)picolinate (159A)

Compound 71C (260 mg, 0.75 mmol) was added to a mixed solvent of dioxane (10 mL) and water (1 mL), and then 1-methyl-5-(4,4,5,5-tetramethyl-1,3,2-dioxin-2-yl)-1H imidazole (312 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 159A (220 mg, 84%).

LC-MS (ESI): m/z=349.1[M+H]⁺.

Step 2:

### 4-(5-(1-methyl-1H-imidazol-5-yl)benzo[d]oxazol-2-yl)picolinic Acid (159B)

Anhydrous methanol (10 mL) and NaOH (126 mg, 3.15 mmol, 2 mL) aqueous solution were successively added to compound 159A (220 mg, 0.63 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent and then obtain a crude product of intermediate 159B (300 mg).

LC-MS (ESI): m/z=321.1 [M+H]⁺.

Step 3:

### (4-(5-(1-methyl-1H-imidazol-5-yl)benzo[d]oxazol-2-yl)pyridin-2-yl)(4-((2-methyl-2H-tetrazol-5-yl)(phenyl)methyl)piperazin-1-yl)methanone

At room temperature, DMF (8 mL), intermediate 2 (162 mg, 0.63 mmol), HATU (359 mg, 0.95 mmol) and DIPEA (244 mg, 1.89 mmol) were successively added to compound 159B (200 mg, 0.63 mmol). The reaction was stirred for 1 hour. The reaction solution was poured into water (50 mL) and extracted with ethyl acetate (30 mL×3). The organic phase was concentrated, and the residue was separated and purified by HPLC to obtain compound 159 (150 mg, 42%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.97 min.

LC-MS (ESI): m/z=561.3 [M+H]⁺.

¹H NMR (400 MHz, DMSO-d₆) δ8.82-8.83 (m, 1H), 8.21-8.22 (m, 1H), 8.16-8.18 (m, 1H), 8.02 (d, 1H), 7.94 (d, 1H), 7.74 (s, 1H), 7.62-7.65 (m, 1H), 7.48-7.50 (m, 2H), 7.34-7.38 (m, 2H), 7.27-7.31 (m, 1H), 7.13 (d, 1H), 5.10 (s, 1H), 4.35 (s, 3H), 3.73 (s, 3H), 3.72-3.73 (m, 2H), 3.49-3.50 (m, 2H), 2.55-2.58 (m, 1H), 2.42-2.48 (m, 2H), 2.31-2.34 (m, 1H).

### Example 160

### (R/S)-(4-((2-(difluoromethyl)-2H-tetrazol-5-yl)(phenyl)-13-methyl)piperazin-1-yl)(4-(6-(1-methyl-1H-imidazol-5-yl)benzo[d]oxazol-2-yl)pyridin-2-yl)methanone

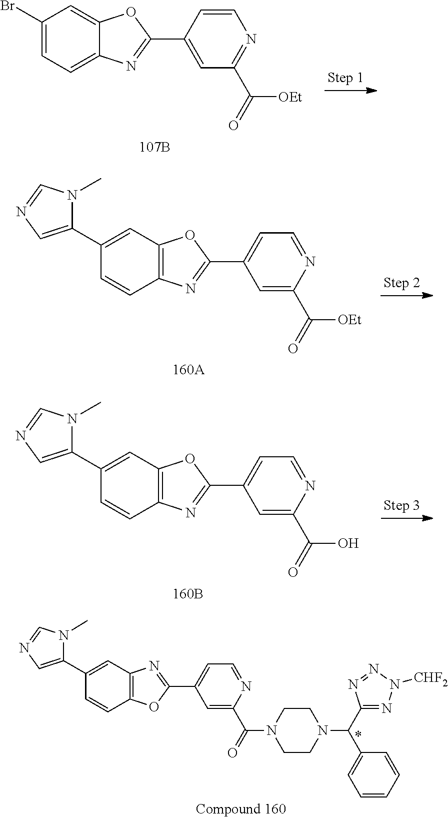 [SMILES: CCOC(=O)c1cc(-c2nc3ccc(Br)cc3o2)ccn1 ; CCOC(=O)c1cc(-c2nc3ccc(-c4cncn4C)cc3o2)ccn1 ; Cn1cncc1-c1ccc2nc(-c3ccnc(C(=O)O)c3)oc2c1 ; Cn1cncc1-c1ccc2oc(-c3ccnc(C(=O)N4CCN(C(c5ccccc5)c5nnn(C(F)F)n5)CC4)c3)nc2c1]

Step 1:

### Ethyl 4-(6-(1-methyl-1H-imidazol-5-yl)benzo[d]oxazol-2-yl)picolinate (160A)

Compound 107B (250 mg, 0.75 mmol) was added to a mixed solvent of dioxane (10 mL) and water (1 mL), and then 1-methyl-4-(4,4,5,5-tetramethyl-1,3,2-dioxin-2-yl)-1H pyrazole (312 mg, 1.50 mmol), potassium carbonate (207 mg, 1.50 mmol) and Pd(dppf)Cl₂ (58 mg, 0.08 mmol) were added. The mixture was warmed to 100° C. and stirred for 7 h under nitrogen protection. The reaction was cooled to room temperature, and the filtrate was concentrated and then separated and purified by silica gel column chromatography (PE:EA=1:4) to obtain 160A (220 mg, 88%).

LC-MS (ESI): m/z=335.1[M+H]⁺.

Step 2:

### 4-(6-(1-methyl-1H-imidazol-5-yl)benzo[d]oxazol-2-yl)picolinic Acid (160B)

Anhydrous methanol (10 mL) and NaOH (132 mg, 3.29 mmol, 2 mL) aqueous solution were successively added to compound 160A (220 mg, 0.66 mmol), and the mixture was stirred at 25° C. for 10 h. TLC or LCMS showed that the reaction of the raw materials was completed. The reaction solution was adjusted to pH=3-4 by dropwise adding 2 N hydrochloric acid and concentrated under reduced pressure to remove the solvent. A small amount of water (2 mL) was added, and the mixture was stirred and then filtered. The filter cake was dried to obtain intermediate 160B (200 mg, 95%).

LC-MS (ESI): m/z=321.1 [M+H]⁺.

Step 3:

### (R/S)-(4-((2-(difluoromethyl)-2H-tetrazol-5-yl)(phenyl)-13-methyl)piperazin-1-yl)(4-(6-(1-methyl-1H-imidazol-5-yl)benzo[d]oxazol-2-yl)pyridin-2-yl)methanone

At room temperature, DMF (8 mL), intermediate 7 (110 mg, 0.38 mmol), HATU (217 mg, 0.57 mmol) and DIPEA (147 mg, 1.14 mmol) were successively added to compound 160B (120 mg, 0.38 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 160 (50 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.32 min.

LC-MS (ESI): m/z=597.3 [M+H]⁺.

¹H NMR (400 MHz, DMSO-d₆) δ9.19 (s, 1H), 8.84-8.86 (m, 1H), 8.56 (t, 1H), 8.23-8.24 (m, 1H), 8.19 (d, 1H), 8.18 (d, 1H), 8.09 (d, 1H), 7.96 (s, 1H), 7.69-7.72 (m, 1H), 7.49-7.51 (m, 2H), 7.38-7.41 (m, 2H), 7.32-7.35 (m, 1H), 5.34 (s, 1H), 3.90 (s, 3H), 3.72-3.73 (m, 2H), 3.53-3.54 (m, 2H), 2.59-2.62 (m, 1H), 2.48-2.50 (m, 2H), 2.36-2.39 (m, 1H).

### Example 161

### (R/S)-(4-((2-(difluoromethyl)-2H-tetrazol-5-yl)(phenyl)methyl)piperazin-1-yl)(4-(5-(1-(trifluoromethyl)-1H-pyrazol-4-yl)benzo[d]oxazol-2-yl)pyridin-2-yl)methanone

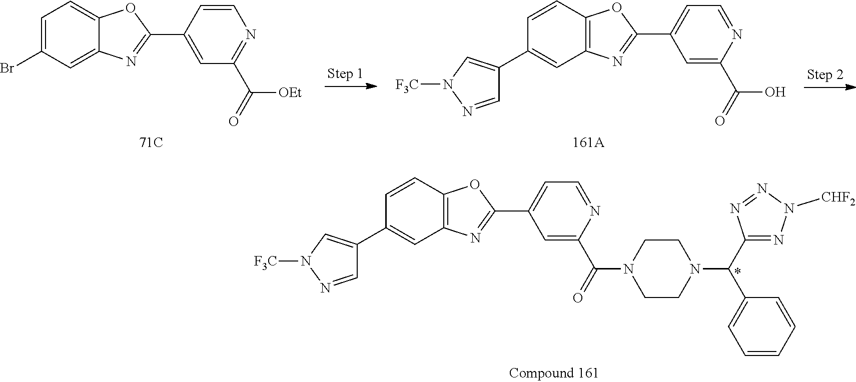 [SMILES: CCOC(=O)c1cc(-c2nc3cc(Br)ccc3o2)ccn1 ; O=C(O)c1cc(-c2nc3cc(-c4cnn(C(F)(F)F)c4)ccc3o2)ccn1 ; O=C(c1cc(-c2nc3cc(-c4cnn(C(F)(F)F)c4)ccc3o2)ccn1)N1CCN(C(c2ccccc2)c2nnn(C(F)F)n2)CC1]

Step 1:

### 4-(5-(1-(trifluoromethyl)-1H-pyrazol-4-yl)benzo[d]oxazol-2-yl)picolinic Acid (161A)

Compound 71C (250 mg, 0.75 mmol) was added to a mixed solvent of dioxane (20 mL) and water (3 mL); 4-(4,4,5,5-tetramethyl-1,3,2-dioxin-2-yl)-1-(trifluoromethyl)-1H pyrazole (197 mg, 0.75 mmol), potassium carbonate (207 mg, 1.50 mmol) and Pd(dppf)Cl₂ (58 mg, 0.08 mmol) were added; and the mixture was warmed to 100° C. and stirred for 5 h under nitrogen protection. The reaction was cooled to room temperature, and the filtrate was concentrated and then separated and purified by silica gel column chromatography (PE:EA=1:4) to obtain 161A (130 mg, 46%).

LC-MS (ESI): m/z=375.1[M+H]⁺.

Step 2:

### (R/S)-(4-((2-(difluoromethyl)-2H-tetrazol-5-yl)(phenyl)methyl)piperazin-1-yl)(4-(5-(1-(trifluoromethyl)-1H-pyrazol-4-yl)benzo[d]oxazol-2-yl)pyridin-2-yl)methanone (Compound 161)

At room temperature, DMF (8 mL), intermediate 7 (102 mg, 0.35 mmol), HATU (217 mg, 0.57 mmol) and DIPEA (147 mg, 1.14 mmol) were successively added to compound 161A (130 mg, 0.35 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 161 (70 mg, 31%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.75 min.

LC-MS (ESI): m/z=651.3 [M+H]⁺.

¹H NMR (400 MHz, DMSO-d₆) δ 9.09 (s, 1H), 8.82-8.83 (m, 1H), 8.60 (s, 1H), 8.56 (t, 1H), 8.32 (s, 1H), 8.16-8.21 (m, 2H), 7.87-7.93 (m, 2H), 7.49-7.51 (m, 2H), 7.38-7.41 (m, 2H), 7.32-7.36 (m, 1H), 5.36 (s, 1H), 3.72-3.73 (m, 2H), 3.52-3.53 (m, 2H), 2.59-2.62 (m, 1H), 2.40-2.45 (m, 2H), 2.33-2.38 (m, 1H).

### Example 162

### (R/S)-(4-((2-(difluoromethyl)-2H-tetrazol-5-yl)(phenyl)methyl)piperazin-1-yl)(4-(5-(1,2-dimethyl-1H-imidazol-5-yl)benzo[d]oxazol-2-yl)pyridin-2-yl)methanone

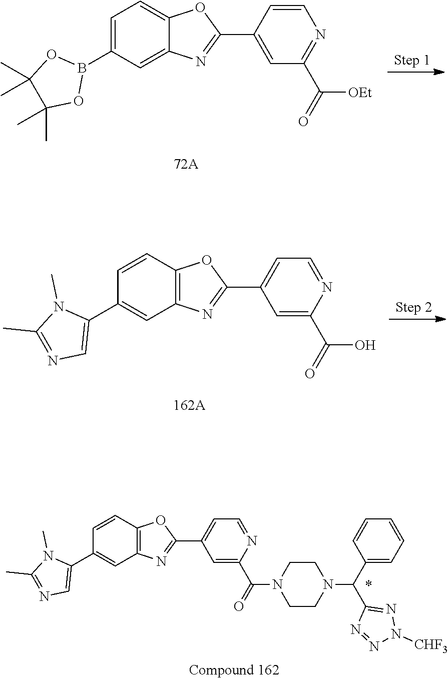

Step 1:

### 4-(5-(1,2-dimethyl-1H-imidazol-5-yl)benzo[d]oxazol-2-yl)picolinic Acid (162A)

Compound 72A (250 mg, 0.75 mmol) was added to a mixed solvent of dioxane (20 mL) and water (3 mL), and then 5-bromo-1,2-dimethyl-1H-imidazole (197 mg, 1.13 mmol), potassium carbonate (207 mg, 1.50 mmol) and Pd(dppf)Cl₂ (58 mg, 0.08 mmol) were added. The mixture was warmed to 100° C. and stirred for 5 h under nitrogen protection. The reaction was cooled to room temperature, and the filtrate was concentrated and then separated and purified by silica gel column chromatography (DCM:MeOH=5:1) to obtain 162A (130 mg, 52%).

LC-MS (ESI): m/z=335.1[M+H]⁺.

Step 2:

### (R/S)-(4-((2-(difluoromethyl)-2H-tetrazol-5-yl)(phenyl)methyl)piperazin-1-yl)(4-(5-(1,2-dimethyl-1H-imidazol-5-yl)benzo[d]oxazol-2-yl)pyridin-2-yl)methanone (Compound 162)

At room temperature, DMF (8 mL), intermediate 7 (114 mg, 0.39 mmol), HATU (217 mg, 0.57 mmol) and DIPEA (147 mg, 1.14 mmol) were successively added to compound 162A (130 mg, 0.39 mmol). The reaction was stirred for 1 hour. The reaction solution was poured into water (50 mL) and extracted with ethyl acetate (20 mL×2). The organic phase was concentrated, and the residue was separated and purified by HPLC to obtain compound 162 (40 mg, 17%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.86 min.

LC-MS (ESI): m/z=611.3 [M+H]⁺.

¹H NMR (400 MHz, DMSO-d₆) δ8.84-8.86 (m, 1H), 8.56 (t, 1H), 8.23-8.24 (m, 1H), 8.18-8.20 (m, 1H), 8.12-8.13 (m, 1H), 8.08 (d, 1H), 7.81 (s, 1H), 7.66-7.69 (m, 1H), 7.49-7.51 (m, 2H), 7.38-7.41 (m, 2H), 7.32-7.35 (m, 1H), 5.34 (s, 1H), 3.72-3.73 (m, 2H), 3.70 (s, 3H), 3.53-3.54 (m, 2H), 2.68 (s, 3H), 2.59-2.62 (m, 1H), 2.48-2.50 (m, 2H), 2.37-2.39 (m, 1H).

### Example 163

### 3,3,3-tideuterio-N-[2-[2-[4-[(R/S)-[5-(difluoromethyl)tetrazol-2-yl]-phenyl-methyl]piperidine-1-carbonyl]-4-pyridyl]-1,3-benzoxazol-5-yl]-2-(trideuteriomethyl)propanamide

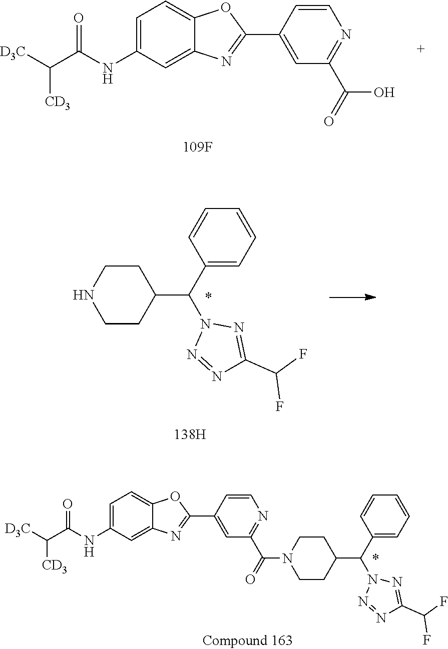 [SMILES: [2H]C([2H])([2H])C(C(=O)Nc1ccc2oc(-c3ccnc(C(=O)O)c3)nc2c1)C([2H])([2H])[2H] ; FC(F)c1nnn(C(c2ccccc2)C2CCNCC2)n1 ; [2H]C([2H])([2H])C(C(=O)Nc1ccc2oc(-c3ccnc(C(=O)N4CCC(C(c5ccccc5)n5nnc(C(F)F)n5)CC4)c3)nc2c1)C([2H])([2H])[2H]]

Compounds 109F and 138H were used as raw materials to obtain compound 163 according to the synthetic method of compound 85.

LC-MS (ESI): m/z=607.2[M+H]⁺.

¹H NMR (400 MHz, CDCl₃) δ 8.74-8.72 (m, 1H), 8.36 (m, 1H), 8.11 (m, 1H), 8.03 (s, 1H), 7.56-7.51 (m, 4H), 7.43-7.36 (m, 4H), 7.08-6.76 (m, 1H), 5.67-5.65 (m, 1H), 4.81-4.77 (m, 2H), 3.98-3.95 (m, 1H), 3.17-3.04 (m, 1H), 2.95-2.79 (m, 1H), 2.53 (s, 1H), 1.58-1.44 (m, 2H), 1.31-1.17 (m, 2H).

### Example 164

### (R/S)—N-(2-(2-(4-((5-(difluoromethyl)-2H-tetrazol-2-yl)(phenyl)methyl)piperidine-1-carbonyl)pyridin-4-yl)benzo[d]oxazol-5-yl)-2-fluoro-2-methylpropanamide

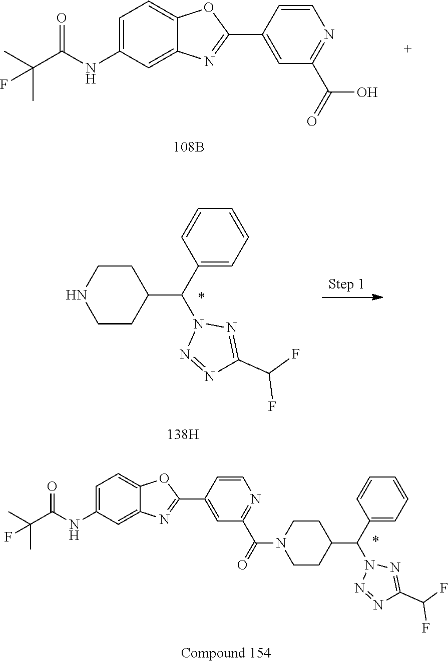 [SMILES: CC(C)(F)C(=O)Nc1ccc2oc(-c3ccnc(C(=O)O)c3)nc2c1 ; FC(F)c1nnn(C(c2ccccc2)C2CCNCC2)n1 ; CC(C)(F)C(=O)Nc1ccc2oc(-c3ccnc(C(=O)N4CCC(C(c5ccccc5)n5nnc(C(F)F)n5)CC4)c3)nc2c1]

Step 1:

### (R/S)—N-(2-(2-(4-((5-(difluoromethyl)-2H-tetrazol-2-yl)(phenyl)methyl)piperidine-1-carbonyl)pyridin-4-yl)benzo[d]oxazol-5-yl)-2-fluoro-2-methylpropanamide

Under nitrogen protection, compound 108B (60 mg, 0.17 mmol), DMF (5 mL), HATU (77.5 mg, 0.20 mmol), DIPEA (33 mg, 0.25 mmol) and 138H (61 mg, 0.21 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 164 as a light yellow solid (40 mg, 37%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.33 min.

¹H NMR (400 MHz, CDCl₃) δ 8.76-8.74 (m, 1H), 8.41 (s, 1H), 8.26-8.24 (m, 1H), 8.16-8.14 (m, 2H), 7.59 (s, 1H), 7.57-7.52 (m, 2H), 7.43-7.36 (m, 3H), 7.08-6.76 (m, 1H), 5.68-5.65 (m, 1H), 4.80-4.76 (m, 1H), 3.98-3.95 (m, 1H), 3.15-3.11 (m, 1H), 2.95-2.82 (m, 2H), 1.73 (s, 3H), 1.67 (s, 3H), 1.62-1.20 (m, 5H).

LC-MS (ESI): m/z=619.2 [M+H]⁺.

### Example 165

### (R/S)-(4-((2-(difluoromethyl)-2H-tetrazol-5-yl)(phenyl)methyl)piperazin-1-yl)(4-(5-(1-methyl-1H-pyrazol-4-yl)benzofuran-2-yl)pyridin-2-yl)methanone

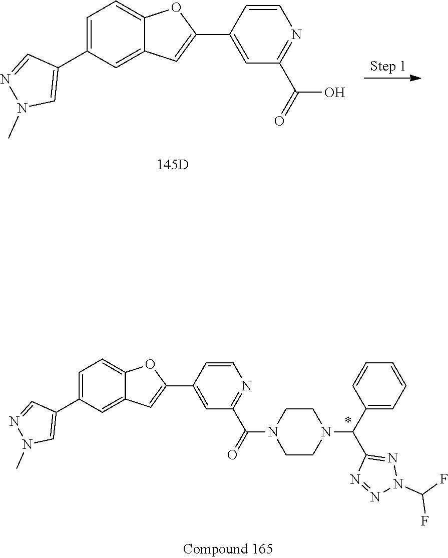 [SMILES: Cn1cc(-c2ccc3oc(-c4ccnc(C(=O)O)c4)cc3c2)cn1 ; Cn1cc(-c2ccc3oc(-c4ccnc(C(=O)N5CCN(C(c6ccccc6)c6nnn(C(F)F)n6)CC5)c4)cc3c2)cn1]

145D and intermediate 7 were used as raw materials to obtain compound 165 according to the synthetic method of compound 85.

¹H NMR (400 MHz, CDCl₃) δ 8.60-8.59 (m, 1H), 8.05 (s, 1H), 7.78-7.70 (m, 4H), 7.63-7.62 (m, 2H), 7.55-7.46 (m, 4H), 7.40-7.31 (m, 3H), 5.17 (s, 1H), 3.97 (s, 3H), 3.92-3.91 (m, 2H), 3.76-3.75 (m, 2H), 2.77-2.48 (m, 4H),

LC-MS (ESI): m/z=596.2 [M+H]⁺.

### Example 166

### (R/S)-(4-((5-(difluoromethyl)-2H-tetrazol-2-yl)(phenyl)methyl)piperidin-1-yl)(4-(5-(1-methyl-1H-imidazol-5-yl)benzo[d]oxazol-2-yl)pyridin-2-yl)methanone

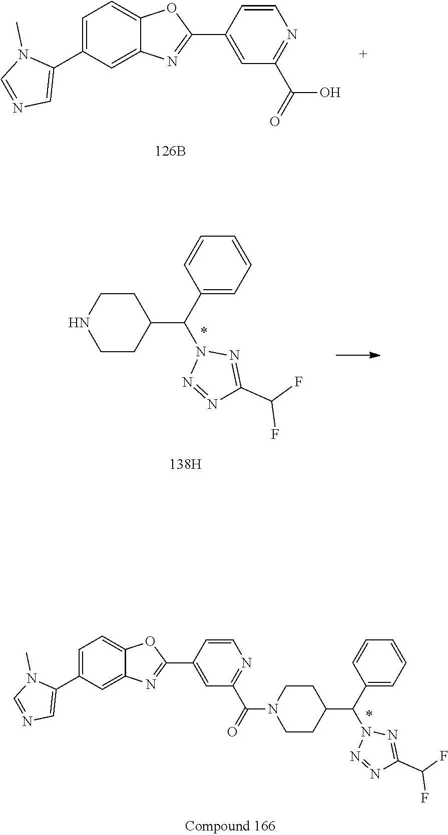 [SMILES: Cn1cncc1-c1ccc2oc(-c3ccnc(C(=O)O)c3)nc2c1 ; FC(F)c1nnn(C(c2ccccc2)C2CCNCC2)n1 ; Cn1cncc1-c1ccc2oc(-c3ccnc(C(=O)N4CCC(C(c5ccccc5)n5nnc(C(F)F)n5)CC4)c3)nc2c1]

126B and 138H were used as raw materials to obtain compound 166 according to the synthetic step of compound 85.

LC-MS (ESI): m/z=596.2 [M+H]⁺.

¹H NMR (400 MHz, CDCl₃) δ 8.77 (t, J=5.3 Hz, 1H), 8.44 (s, 1H), 8.23 (s, 1H), 8.16-8.11 (m, 1H), 7.86 (d, J=1.1 Hz, 1H), 7.75 (d, J=8.5 Hz, 1H), 7.61-7.47 (m, 3H), 7.47-7.28 (m, 4H), 7.11-6.74 (m, 1H), 5.66 (d, J=11.0 Hz, 1H), 4.79 (d, J=12.8 Hz, 1H), 4.11-4.00 (m, 1H), 3.81 (s, 3H), 3.14-2.85 (m, 3H), 1.65-1.10 (m, 4H).

### Example 167

### (R/S)-(4-(5-(1-methyl-1H-imidazol-5-yl)benzo[d]oxazol-2-yl)pyridin-2-yl)(4-((5-methyl-2H-tetrazol-2-yl)(phenyl)methyl)piperidin-1-yl)methanone

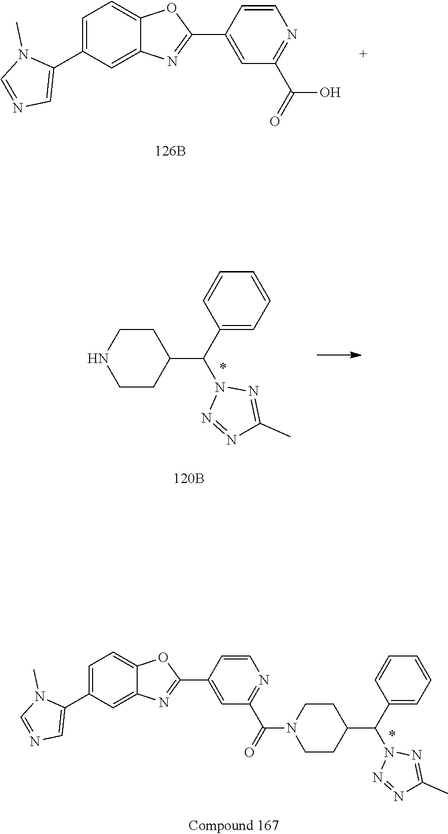 [SMILES: Cn1cncc1-c1ccc2oc(-c3ccnc(C(=O)O)c3)nc2c1 ; Cc1nnn(C(c2ccccc2)C2CCNCC2)n1 ; Cc1nnn(C(c2ccccc2)C2CCN(C(=O)c3cc(-c4nc5cc(-c6cncn6C)ccc5o4)ccn3)CC2)n1]

126B and 120B were used as raw materials to obtain compound 167 according to the synthetic step of compound 85.

LC-MS (ESI): m/z=560.2 [M+H]⁺.

¹H NMR (400 MHz, CDCl₃) δ 8.77 (t, J=4.9 Hz, 1H), 8.42 (s, 1H), 8.15-8.11 (m, 1H), 8.08 (s, 1H), 7.84 (s, 1H), 7.73 (d, J=8.5 Hz, 1H), 7.58-7.45 (m, 3H), 7.44-7.26 (m, 4H), 5.52 (d, J=10.7 Hz, 1H), 4.77 (s, 1H), 4.01 (d, J=13.5 Hz, 1H), 3.78 (s, 3H), 3.16-3.04 (m, 1H), 2.95-2.74 (m, 2H), 2.53 (d, J=19.5 Hz, 3H), 1.63-1.28 (m, 4H).

### Examples 168 and 169

### (1S,2S)—N-(2-(2-(4-((R/S)-(2-(difluoromethyl)-2H-tetrazol-5-yl)(phenyl)methyl)piperidine-1-carbonyl)pyridin-4-yl)benzo[d]oxazol-5-yl)-2-fluorocyclopropane-1-carboxamide

### (1S,2S)—N-(2-(2-(4-((S/R)-(2-(difluoromethyl)-2H-tetrazol-5-yl)(phenyl)methyl)piperidine-1-carbonyl)pyridin-4-yl)benzo[d]oxazol-5-yl)-2-fluorocyclopropane-1-carboxamide

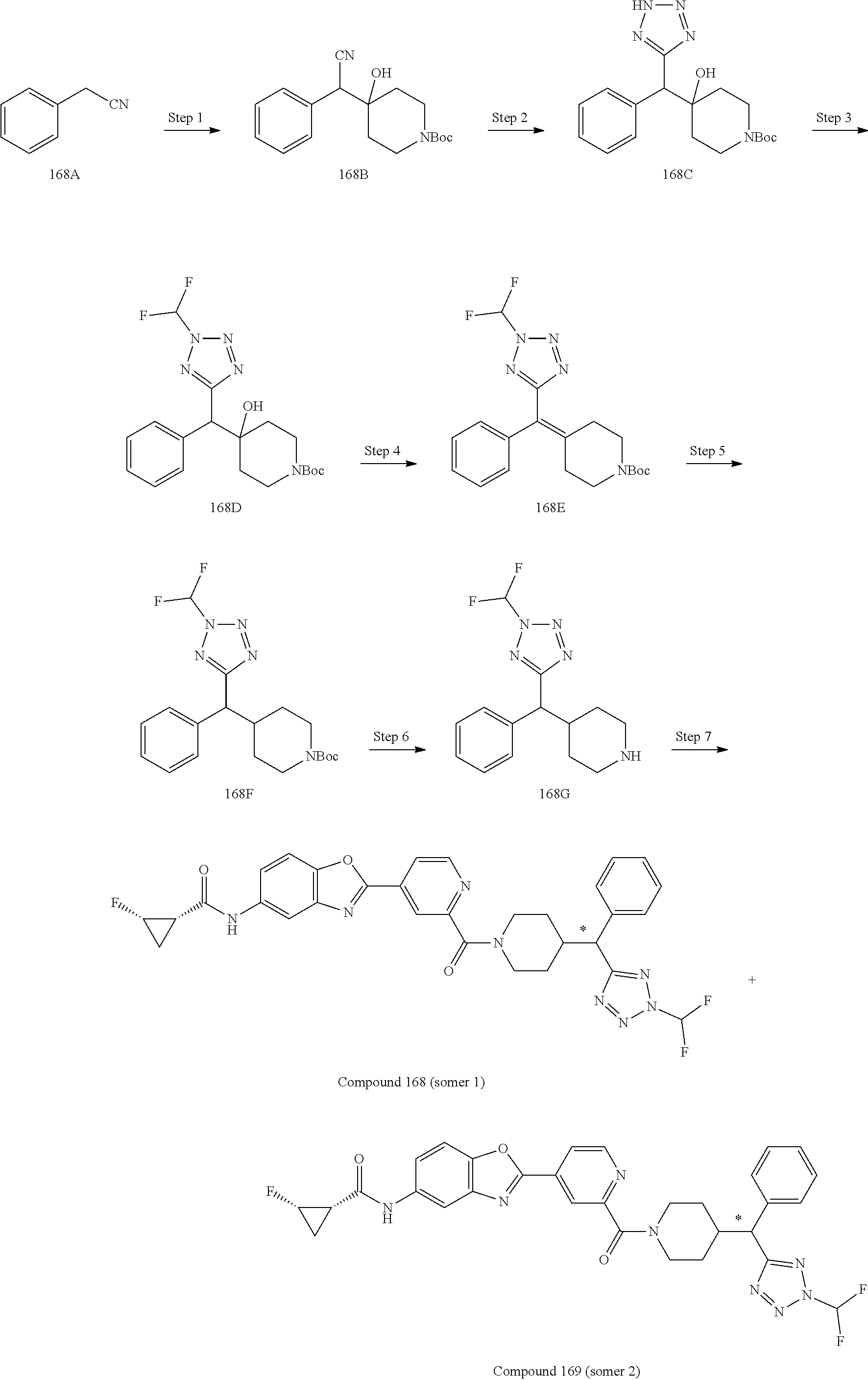

Step 1:

### tert-butyl 4-(cyano(phenyl)methyl)-4-hydroxypiperidine-1-carboxylate (168B)

Phenylacetonitrile (3.0 g, 25.6 mmol) was dissolved in THE (40 mL) under N₂ protection. The resulting solution was cooled to −20° C., and LiHMDS (50 ml, 1 mol/L) was added dropwise. After completion of the dropwise addition, the reaction solution was stirred at −20° C. for 1 hour, and N-Boc piperidone (5.5 g, 27.5 mmol) was added in portions. After the addition was completed, the reaction was allowed to naturally warm to room temperature and stirred for 1 hour. The reaction solution was poured into water, extracted with ethyl acetate, washed with saturated brine, dried over anhydrous sodium sulfate and concentrated to obtain a crude product, which was subjected to column chromatography (petroleum ether:ethyl acetate=3:1) to obtain compound 168B (2.5 g, 30.9%).

LC-MS (ESI): m/z=317.2[M+H]⁺.

Step 2:

### tert-butyl 4-hydroxy-4-(phenyl(2H-tetrazol-5-yl)methyl)piperidine-1-carboxylate (168C)

168B (1.0 g, 3.16 mmol) and triethylamine hydrochloride (0.8 g, 6 mmol) were added to toluene (20 ml) at room temperature, and NaN₃ (0.39 g, 6 mmol) was added in portions. After the addition was completed, the reaction was warmed to 100° C. and stirred overnight. After 20 hours, the heating was stopped, and the mixture was naturally cooled to room temperature and then concentrated to remove most of toluene. The residue was diluted by adding ethyl acetate, washed with water and saturated brine, dried over anhydrous sodium sulfate, concentrated under reduced pressure and then purified by column chromatography (petroleum ether:ethyl acetate=2:1) to obtain 168C (0.7 g, 63.2%).

LC-MS (ESI): m/z=360.2[M+H]⁺.

Step 3:

### tert-butyl 4-((2-(difluoromethyl)-2H-tetrazol-5-yl)(phenyl)methyl)-4-hydroxypiperidine-1-carboxylate (168D)

168C (600 mg, 1.67 mmol) was dissolved in water (10 mL) and acetonitrile (10 ml) at room temperature, and potassium hydroxide (560 mg, 10 mmol) was added in portions under ice-water bath cooling. After the addition was completed, diethyl (bromodifluoromethyl)phosphonate (567 mg, 2.0 mmol) was added dropwise. The reaction was stirred for 1 hour; the reaction solution was diluted with water, extracted with ethyl acetate, washed with saturated brine, dried over anhydrous sodium sulfate and concentrated; and then the residue was subjected to column chromatography (PE:EA=5:1) to obtain 168D (320 mg, 48.8%).

LC-MS (ESI): m/z=410.2 [M+H]⁺.

Step 4:

### tert-butyl 4-((2-(difluoromethyl)-2H-tetrazol-5-yl)(phenyl)methylene)piperidine-1-carboxylate (168E)

168D (270 mg, 0.66 mmol) was added to pyridine (2 ml) at room temperature, and the mixture was cooled in an ice-water bath. SOCl₂ (0.3 ml) was slowly added dropwise. After stirred for 1 hour, the reaction was slowly poured into ice water, and the solution was extracted with ethyl acetate, washed with saturated brine, dried over anhydrous sodium sulfate and concentrated to obtain 168E (220 mg, 84.2%).

LC-MS (ESI): m/z=392.2 [M+H]⁺.

Step 5:

### tert-butyl 4-((2-(difluoromethyl)-2H-tetrazol-5-yl)(phenyl)methyl)piperidine-1-carboxylate (168F)

168E (200 mg, 0.52 mmol) was added to methanol (2 ml), and a catalytic amount of 10% palladium on carbon catalyst was added. The mixture was subjected to hydrogen replacement 3 times, and then the reaction was stirred under hydrogen atmosphere for 16 hours. The reaction solution was filtered through a small amount of celite. The filtrate was concentrated to obtain 168F (180 mg, 80%).

LC-MS (ESI): m/z=394.2 [M+H]⁺.

Step 6:

### 4-((2-(difluoromethyl)-2H-tetrazol-5-yl)(phenyl)methyl)piperidine (168G)

168F (160 mg, 0.40 mmol) was added to ethyl acetate (2 ml), and 6 mol/L HCl/dioxane (2 ml) was added dropwise. The mixture was stirred at room temperature for 2 hours and then concentrated to dryness to obtain the hydrochloride of compound 168G (100 mg, 80.5%), which was directly used in the next reaction.

LC-MS (ESI): m/z=294.2 [M+H]⁺.

Step 7:

### (1S,2S)—N-(2-(2-(4-((R/S)-(2-(difluoromethyl)-2H-tetrazol-5-yl)(phenyl)methyl)piperidine-1-carbonyl)pyridin-4-yl)benzo[d]oxazol-5-yl)-2-fluorocyclopropane-1-carboxamide (Compound 168)

### (1S,2S)—N-(2-(2-(4-((S/R)-(2-(difluoromethyl)-2H-tetrazol-5-yl)(phenyl)methyl)piperidine-1-carbonyl)pyridin-4-yl)benzo[d]oxazol-5-yl)-2-fluorocyclopropane-1-carboxamide (Compound 169)

168G (100 mg, 0.34 mmol) and 117F (116 mg, 0.34 mmol) were added to dichloromethane (2 ml); DIEA (72 mg, 0.6 mmol) was added dropwise; and HATU (152 mg, 0.4 mmol) was added in portions. The mixture was stirred at room temperature for 2 hours; the reaction was poured into water, extracted with dichloromethane, washed with saturated brine, dried over anhydrous sodium sulfate and concentrated; and then the residue was subjected to column chromatography (PE:EA=5:1) to obtain the crude product (100 mg, 47.7%). The products were purified and separated by chiral HPLC (instrument name: MG II preparative SFC (SFC-1); chromatographic column: ChiralCel OJ, 250×30 mm I.D., 5 μm; mobile phase: A for CO₂ and B for isopropanol; gradient: B 40%; flow rate: 60 mL/min; column pressure: 100 bar; column temperature: 38° C.; absorption wavelength: 220 nm; cycle time: about 17 min) to obtain compound 168 (32 mg) and compound 169 (30 mg). Retention time for compound 168: 2.098 min; retention time for compound 169: 4.692 min. Compounds 168 and 169 were compounds in single configurations.

¹H NMR (400 MHz, DMSO) δ10.47 (s, 1H), 8.81-8.79 (m, 1H), 8.69-8.36 (m, 1H), 8.24 (s, 1H), 8.16 (s, 1H), 8.14-8.12 (m, 1H), 7.80 (s, 1H), 7.64-7.61 (m, 1H), 7.48-7.23 (m, 5H), 4.88-4.86 (m, 1H), 4.54-4.44 (m, 1H), 4.36 (s, 1H), 3.69 (s, 1H), 3.10-3.02 (m, 1H), 2.87-2.77 (s, 1H), 2.11-2.08 (m, 1H), 1.67-1.61 (m, 1H), 1.57-1.46 (m, 1H), 1.33-1.15 (m, 5H).

LC-MS (ESI): m/z=617.2 [M+H]⁺.

### Example 170 and Example 171

### (R/S)-(4-(5-(1-(difluoromethyl)-1H-imidazol-4-yl)benzo[d]oxazol-2-yl)pyridin-2-yl)(4-((2-(difluoromethyl)-2H-tetrazol-5-yl)(phenyl)methyl)piperazin-1-yl)methanone (Compound 170) and

### (R/S)-(4-(5-(1-(difluoromethyl)-1H-imidazol-5-yl)benzo[d]oxazol-2-yl)pyridin-2-yl)(4-((2-(difluoromethyl)-2H-tetrazol-5-yl)(phenyl)methyl)piperazin-1-yl)methanone (Compound 171)

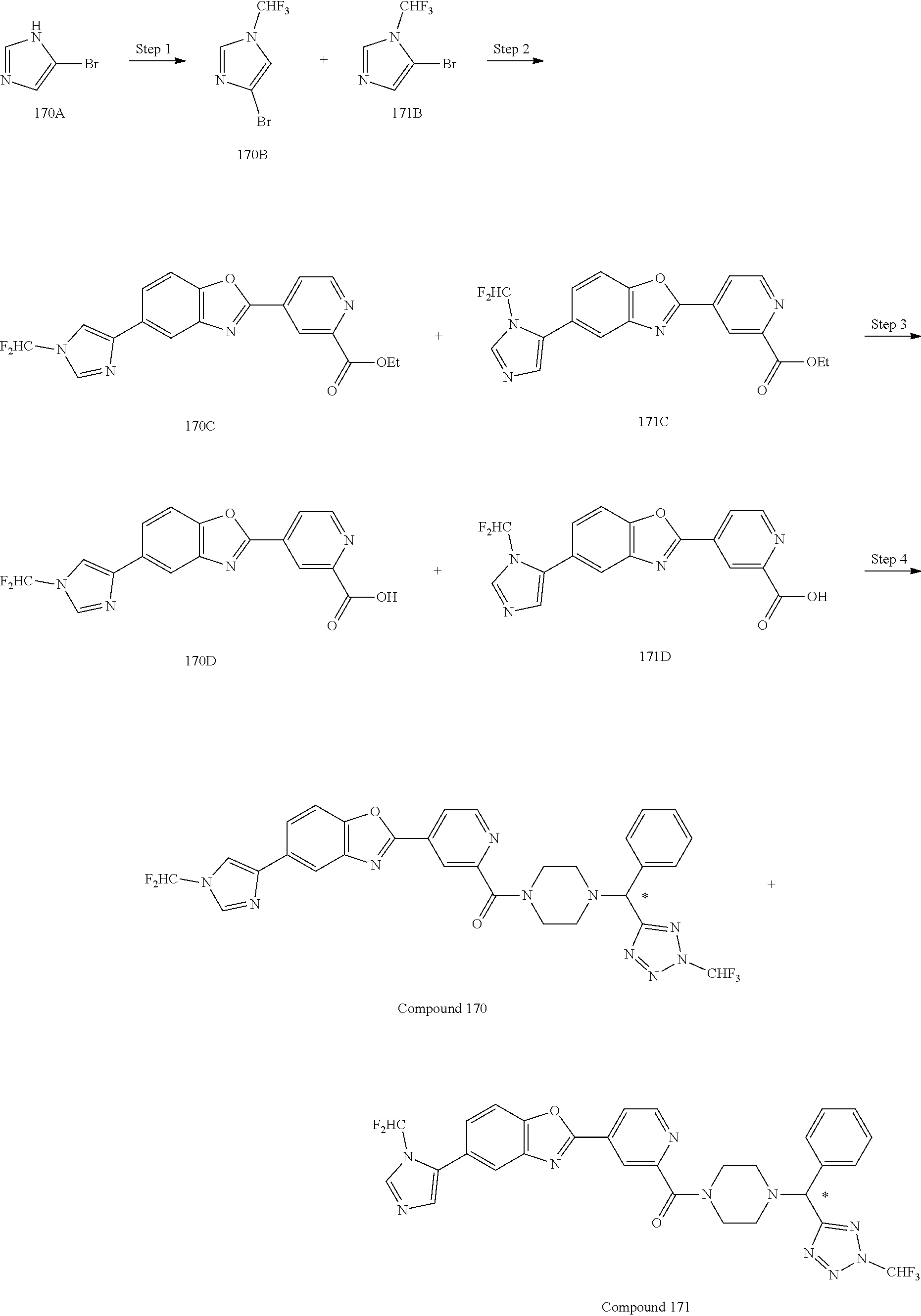

Step 1:

### 4-bromo-1-(difluoromethyl)-1H-imidazole (170B)

### 5-bromo-1-(difluoromethyl)-1H-imidazole (171B)

Compound 5-bromo-1H-imidazole (3000 mg, 20.4 mmol) was added to DMF (50 mL) solvent, and then 2-chloro-2,2-sodium difluoroacetate (9302 mg, 61.2 mmol) and potassium carbonate (8448 mg, 61.2 mmol) were added. The mixture was warmed to 130° C. and stirred for 5 h. The reaction was cooled to room temperature and filtered. The filtrate was concentrated and then separated and purified by silica gel column chromatography (DCM:MeOH=10:1) to obtain a mixture of 170B and 171B (1300 mg, 84%).

LC-MS (ESI): m/z=196.9[M+H]⁺.

Step 2:

### Ethyl 4-(5-(1-(difluoromethyl)-1H-imidazol-4-yl)benzo[d]oxazol-2-yl)picolinate (170C)

### Ethyl 4-(5-(1-(difluoromethyl)-1H-imidazol-5-yl)benzo[d]oxazol-2-yl)picolinate (171C)

A mixture (500 mg, 2.54 mmol) of compound 170B and 171B was added to a mixed solvent of dioxane (20 mL) and water (1 mL), and then ethyl 4-(4,4,5,5-tetramethyl-1,3,2-dioxaborolan-2-yl)benzo[d]oxazol-2-yl)picolinate (1501 mg, 3.81 mmol), potassium carbonate (701 mg, 5.08 mmol) and Pd(dppf)Cl₂ (91 mg, 0.13 mmol) were added. The mixture was warmed to 100° C. and stirred for 5 h under nitrogen protection. The reaction was cooled to room temperature and filtered. The filtrate was concentrated and then separated and purified by silica gel column chromatography (PE:EA=1:4) to obtain a mixture of 170C and 171C (200 mg, 21%).

LC-MS (ESI): m/z=385.1[M+H]⁺.

Step 3:

### 4-(5-(1-(difluoromethyl)-1H-imidazol-4-yl)benzo[d]oxazol-2-yl)picolinic Acid (170D)

### 4-(5-(1-(difluoromethyl)-1H-imidazol-5-yl)benzo[d]oxazol-2-yl)picolinic Acid (171D)

Anhydrous methanol (10 mL) and NaOH (83 mg, 2.08 mmol, 2 mL) aqueous solution were successively added to a mixture of compounds 170C and 171C (200 mg, 0.52 mmol), and the reaction was stirred at 25° C. for 3 h. TLC or LCMS showed that the reaction of the raw materials was completed. The reaction solution was adjusted to pH=3-4 by dropwise adding 2 N hydrochloric acid and concentrated under reduced pressure to remove the solvent and then obtain a crude product. The crude product was washed with a small amount of water, and then the solid was dried to obtain a mixture of intermediates 170D and 171D (130 mg, 70%).

LC-MS (ESI): m/z=357.1 [M+H]⁺.

Step 4:

### (R/S)-(4-(5-(1-(difluoromethyl)-1H-imidazol-4-yl)benzo[d]oxazol-2-yl)pyridin-2-yl)(4-((2-(difluoromethyl)-2H-tetrazol-5-yl)(phenyl)methyl)piperazin-1-yl)methanone (Compound 170)

### (R/S)-(4-(5-(1-(difluoromethyl)-1H-imidazol-5-yl)benzo[d]oxazol-2-yl)pyridin-2-yl)(4-((2-(difluoromethyl)-2H-tetrazol-5-yl)(phenyl)methyl)piperazin-1-yl)methanone (Compound 171)

At room temperature, DMF (8 mL), intermediate (109 mg, 0.37 mmol), HATU (211 mg, 0.56 mmol) and DIPEA (143 mg, 1.11 mmol) were successively added to a mixture of compounds 170D and 171D (130 mg, 0.37 mmol). The reaction was stirred for 1 hour. The reaction solution was poured into water (50 mL) and extracted with ethyl acetate (30 mL×3). The organic phase was concentrated, and the residue was separated and purified by HPLC to obtain compound 170 (110 mg) and compound 171 (25 mg). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.04 min for compound 170 and 11.85 min for compound 171.

### Compound 170

LC-MS (ESI): m/z=633.3 [M+H]⁺.

¹H NMR (400 MHz, DMSO-d₆) δ 8.83-8.85 (m, 1H), 8.56 (t, 1H), 8.50 (s, 1H), 8.24-8.25 (m, 1H), 8.18-8.20 (m, 1H), 8.01-8.02 (m, 2H), 7.86 (t, 1H), 7.63-7.66 (m, 1H), 7.53-7.51 (m, 2H), 7.39-7.43 (m, 3H), 7.33-7.37 (m, 1H), 5.44 (s, 1H), 3.75-3.76 (m, 2H), 3.56-3.57 (m, 2H), 2.66-2.69 (m, 1H), 2.56-2.59 (m, 2H), 2.44-2.46 (m, 1H).

### Compound 171

LC-MS (ESI): m/z=633.3 [M+H]⁺.

¹H NMR (400 MHz, DMSO-d₆) δ 8.81-8.82 (m, 1H), 8.55 (t, 1H), 8.32-8.33 (m, 1H), 8.25-8.26 (m, 2H), 8.20-8.21 (m, 1H), 8.16-8.17 (m, 1H), 8.04-8.06 (m, 1H), 7.89 (t, 1H), 7.86-7.87 (m, 1H), 7.49-7.50 (m, 2H), 7.37-7.41 (m, 2H), 7.31-7.35 (m, 1H), 5.32 (s, 1H), 3.72-3.73 (m, 2H), 3.49-3.50 (m, 2H), 2.55-2.58 (m, 1H), 2.42-2.48 (m, 2H), 2.33-2.37 (m, 1H).

### Example 172

### (R/S)-(4-((5-(difluoromethyl)-2H-tetrazol-2-yl)(phenyl)methyl)piperidin-1-yl)(4-(5-(1-methyl-1H-pyrazol-4-yl)benzo[d]oxazol-2-yl)pyridin-2-yl)methanone

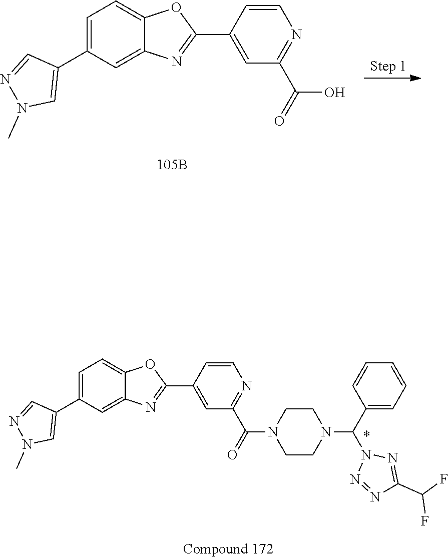 [SMILES: Cn1cc(-c2ccc3oc(-c4ccnc(C(=O)O)c4)nc3c2)cn1 ; Cn1cc(-c2ccc3oc(-c4ccnc(C(=O)N5CCN(C(c6ccccc6)n6nnc(C(F)F)n6)CC5)c4)nc3c2)cn1]

105B and 138H were used as raw materials to obtain compound 172 according to the synthetic method of compound 138.

¹H NMR (400 MHz, CDCl₃) δ 8.58-8.57 (m, 1H), 7.75-7.70 (m, 4H), 7.63-7.62 (m, 2H), 7.50-7.46 (m, 4H), 7.38-7.31 (m, 3H), 5.21 (s, 1H), 3.99 (s, 3H), 3.92-3.91 (m, 2H), 3.76-3.75 (m, 2H), 2.77-2.48 (m, 4H),

LC-MS (ESI): m/z=597.2 [M+H]⁺.

### Example 173

### N-(1-(4-(5-((1S,2S)-2-fluorocyclopropane-1-carboxamido)benzo[d]oxazol-2-yl)picolinoyl)piperidin-4-yl)-1-methyl-N-phenyl-1H-pyrazole-3-carboxamide

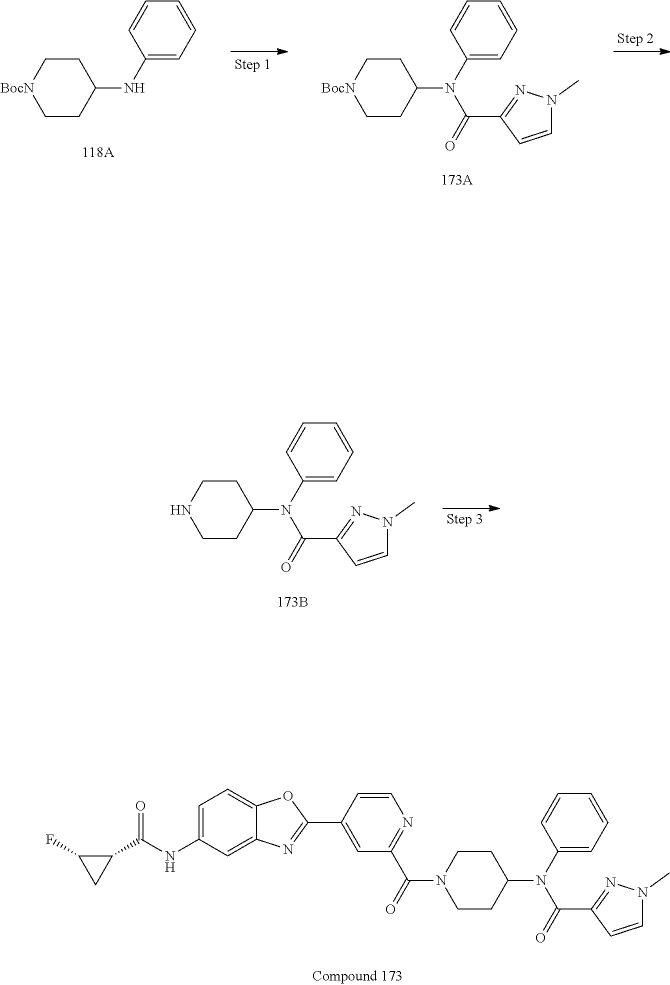 [SMILES: CC(C)(C)OC(=O)N1CCC(Nc2ccccc2)CC1 ; Cn1ccc(C(=O)N(c2ccccc2)C2CCN(C(=O)OC(C)(C)C)CC2)n1 ; Cn1ccc(C(=O)N(c2ccccc2)C2CCNCC2)n1 ; Cn1ccc(C(=O)N(c2ccccc2)C2CCN(C(=O)c3cc(-c4nc5cc(NC(=O)[C@@H]6C[C@@H]6F)ccc5o4)ccn3)CC2)n1]

Step 1:

### tert-buty-(1-methyl-N-phenyl-1H-pyrazole-3-carboxamido)piperidine-1-carboxylate (173A)

To a single-necked flask, 1-methylpyrazole-3-carboxylic acid (0.13 g, 1 mmol) and toluene (15 mL) were successively added, and thionyl chloride (2 mL) was added dropwise under an ice bath. After the dropwise addition was completed, the reaction was stirred at 100° C. for 2 hours. The reaction solution was cooled to room temperature and concentrated under reduced pressure to remove excess thionyl chloride to obtain 1-methylpyrazole-3-carbonyl chloride. 118A (0.3 g, 1.1 mmol) was dissolved in anhydrous dichloromethane (20 mL), and triethylamine (150 mg, 1.5 mmol) was added. The acid chloride prepared above was added dropwise under an ice bath. After the dropwise addition was completed, the reaction was stirred for 2 hours. After LCMS showed that the reaction was completed, the reaction solution was washed successively with distilled water (100 mL×2) and saturated brine (50 mL). The organic phase was concentrated under reduced pressure, and the residue was separated by column chromatography (eluent: EA/PE=1/4) to obtain target compound 173A (0.29 g, 69.5%).

LC-MS (ESI): m/z=385.1[M+H]⁺.

Step 2:

### 1-methyl-N-phenyl-N-(piperidin-4-yl)-1H-pyrazole-3-carboxamide (173B)

Compound 173A (90 mg, 0.23 mmol), dichloromethane (5 mL) and trifluoroacetic acid (2 mL) were successively added to a single-necked flask. The reaction was stirred at room temperature for 2 h and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure, and then the residue was separated and purified by silica gel column chromatography (eluent: DCM/MeOH=1/60) to obtain compound 173B (60 mg, 90%).

LC-MS (ESI): m/z=285.3[M+H]⁺.

Step 3:

### N-(1-(4-(5-((1S,2S)-2-fluorocyclopropane-1-carboxamido)benzo[d]oxazol-2-yl)picolinoyl)piperidin-4-yl)-1-methyl-N-phenyl-1H-pyrazole-3-carboxamide (Compound 173)

Under nitrogen protection, compound 117F (72 mg, 0.21 mmol), DMF (5 mL), HATU (96 mg, 0.25 mmol), DIPEA (58 mg, 0.45 mmol) and 173B (60 mg, 0.21 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 173 as a light yellow solid (30 mg, 23.4%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.05 min.

¹H NMR (400 MHz, CDCl₃) δ 8.73-8.72 (m, 1H), 8.25 (s, 1H), 8.11-8.09 (m, 1H), 7.99 (s, 1H), 7.93 (s, 1H), 7.55-7.47 (m, 2H), 7.36-7.35 (m, 3H), 7.13-7.11 (m, 2H), 7.02 (s, 1H), 5.50 (s, 1H), 5.14-5.08 (m, 1H), 4.90-4.73 (m, 2H), 3.96-3.93 (m, 1H), 3.78 (s, 3H), 3.39-3.32 (m, 3H), 3.02-2.97 (m, 1H), 2.51 (s, 2H), 2.12-2.08 (m, 1H), 1.98-1.83 (m, 2H), 1.65-1.55 (m, 2H).

LC-MS (ESI): m/z=608.3 [M+H]⁺.

### Example 174

### (R/S)-(4-((2-(difluoromethyl)-2H-tetrazol-5-yl)(phenyl)methyl)piperazin-1-yl)(4-(5-(isoxazol-4-yl)benzo[d]oxazol-2-yl)pyridin-2-yl)methanone

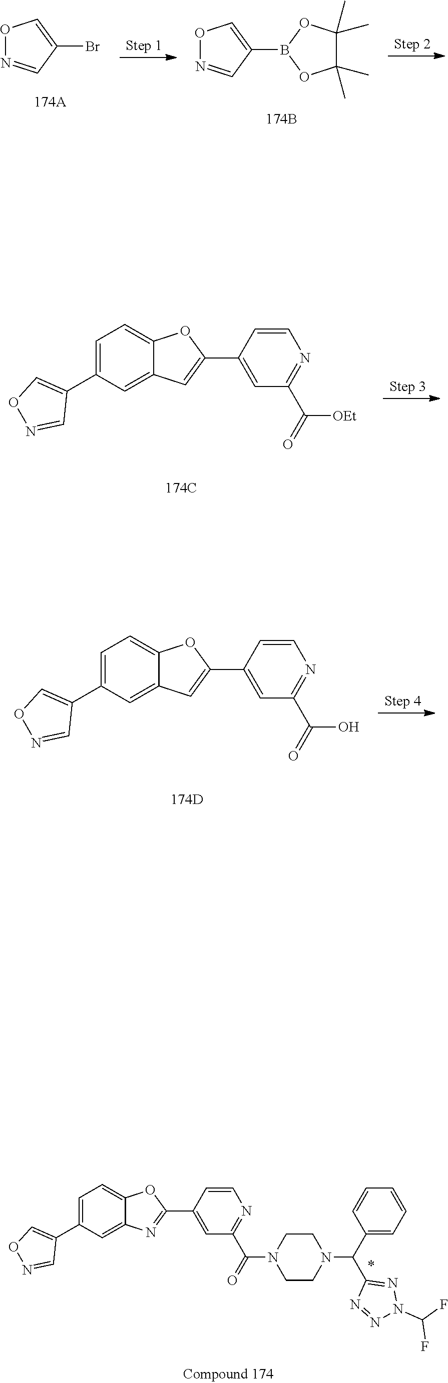 [SMILES: Brc1cnoc1 ; CC1(C)OB(c2cnoc2)OC1(C)C ; CCOC(=O)c1cc(-c2cc3cc(-c4cnoc4)ccc3o2)ccn1 ; O=C(O)c1cc(-c2cc3cc(-c4cnoc4)ccc3o2)ccn1 ; O=C(c1cc(-c2nc3cc(-c4cnoc4)ccc3o2)ccn1)N1CCN(C(c2ccccc2)c2nnn(C(F)F)n2)CC1]

Step 1:

### 4-(4,4,5,5-tetramethyl-1,3,2-dioxaborolan-2-yl)isoxazole (174B)

Compound 174A (3.5 g, 23.65 mmol) was dissolved in tetrahydrofuran (100 mL); triisopropyl borate (13.4 g, 70.95 mmol) was added; and n-butyl lithium (11.5 ml, 2.5 M, 28.38 mmol) was added dropwise at −78° C. under nitrogen protection. The mixture was reacted at room temperature overnight, and then hydrochloric acid (28.5 mL, 1 N, 28.38 mmol) was added, and the reaction solution was extracted with ethyl acetate (100 mL×3) and washed twice with saturated brine. The organic phase was dried and concentrated; the obtained crude product was dissolved in tetrahydrofuran (100 mL); and pinacol (8.3 g, 70.95 mmol) was added. The resulting mixture was reacted at room temperature overnight, dried and concentrated, and then the residue was separated and purified by silica gel column chromatography (EA:PE=10%-20%) to obtain 174B (600 mg, 13%).

LC-MS (ESI): m/z=196.0 [M+H]⁺.

Step 2:

### Ethyl 4-(5-(isoxazol-4-yl)benzo[d]oxazol-2-yl)picolinate (174C)

Compound 174B (600 mg, 3.1 mmol) was dissolved in dioxane (50 mL) at room temperature, and ethyl 4-(5-bromobenzo[d]oxazol-2-yl)picolinate (1.0 g, 2.9 mmol), potassium carbonate (0.6 g, 4.4 mmol), water (5 mL) and [1,1′-bis(diphenylphosphino)ferrocene]palladium dichloride dichloromethane complex (100 mg) were successively added. Under nitrogen protection, the mixture was stirred at 80° C. for 2 hours, filtered and concentrated under reduced pressure, and the residue was separated and purified by silica gel column chromatography (EA:PE=10%-20%) to obtain 174C (500 mg, 51%).

LC-MS (ESI): m/z=336.1 [M+H]⁺.

Step 3:

### 4-(5-(isoxazol-4-yl)benzo[d]oxazol-2-yl)picolinic Acid (174D)

At room temperature, compound 174C (500 mg, 1.5 mmol) was dissolved in methanol (20 mL), and lithium hydroxide (500 mg) was dissolved in 20 mL of pure water. An aqueous solution of lithium hydroxide was added to the reaction solution; the mixture was stirred at 40° C. for 0.5 hours and then adjusted to pH 6-7 with 2 N hydrochloric acid; and the resulting solution was extracted with ethyl acetate (30 mL×3). The combined organic phase was dried over anhydrous sodium sulfate and concentrated under reduced pressure to obtain 174D (400 mg, 87%).

LC-MS (ESI): m/z=308.1 [M+H]⁺.

Step 4:

### (R/S)-(4-((2-(difluoromethyl)-2H-tetrazol-5-yl)(phenyl)methyl)piperazin-1-yl)(4-(5-(isoxazol-4-yl)benzo[d]oxazol-2-yl)pyridin-2-yl)methanone (Compound 174)

DMF (20 mL), intermediate 7 (376 mg, 1.14 mmol), HATU (570 mg, 1.5 mmol) and DIEA (387 mg, 3.0 mmol) were successively added to compound 174D (350 mg, 1.14 mmol), and the mixture was stirred at room temperature for 5 h. The reaction was quenched by adding water, extracted 3 times with ethyl acetate and washed twice with saturated brine. The organic phase was dried and concentrated to obtain a crude product compound, which was subjected to preparative chromatography to obtain compound 174 (10 mg, 1.5%). Preparative separation conditions: instrument: waters 2767 (preparative liquid phase chromatographic instrument); chromatographic column: XBridge@ Prep C18 (19 mm×250 mm); The sample was dissolved in DMF and filtered with a 0.45 m filter to prepare a sample solution. Preparative chromatography conditions: mobile phase A: acetonitrile; mobile phase B: water (containing 0.5% ammonia water); gradient elution: the content of mobile phase A rises from 45% to 75%; flow rate: 15 ml/min; elution time: 20 min; retention time: about 14 min;

LC-MS (ESI): m/z=584.2 [M+H]⁺.

¹H NMR (400 MHz, CDCl₃) δ 8.78-8.69 (m, 2H), 8.61 (s, 1H), 8.44 (s, 1H), 8.17-8.07 (m, 1H), 7.91 (s, 1H), 7.77-7.48 (m, 5H), 7.41-7.31 (m, 3H), 5.19 (s, 1H), 4.02-3.65 (m, 4H), 2.86-2.44 (m, 4H).

### Example 175

### N-(2-(2-(3-((2-(difluoromethyl)-2H-tetrazol-5-yl)(phenyl)methyl)-3,8-diazabicyclo[3.2.1]octane-8-carbonyl)pyridin-4-yl)benzo[d]oxazol-5-yl)cyclopropanecarboxamide

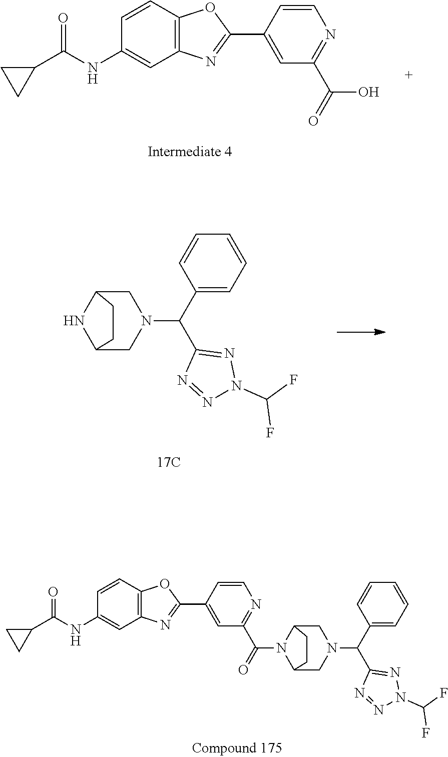 [SMILES: O=C(O)c1cc(-c2nc3cc(NC(=O)C4CC4)ccc3o2)ccn1 ; FC(F)n1nnc(C(c2ccccc2)N2CC3CCC(C2)N3)n1 ; O=C(Nc1ccc2oc(-c3ccnc(C(=O)N4C5CCC4CN(C(c4ccccc4)c4nnn(C(F)F)n4)C5)c3)nc2c1)C1CC1]

Intermediate 4 (53 mg, 0.16 mmol) was dissolved in N,N-dimethylformamide (2 mL), and the mixture was cooled to 0° C.; HATU (75 mg, 0.2 mmol) and DIPEA (68 mg, 0.525 mmol) were added; and then compound 17C (60 mg, 0.16 mmol) was added. After the addition, the mixture was stirred for 1 hour while the temperature was controlled at 0° C., and then the reaction was quenched by adding ice water (10 mL). The resulting solution was extracted twice with dichloromethane (50 mL×2). The organic phase was combined, dried over anhydrous sodium sulfate and concentrated under reduced pressure, and then the residue was separated by column chromatography (DCM:MeOH=20:1) to obtain compound 33 (40 mg, 40%).

¹H NMR (400 MHz, CD₃OD) δ 8.80-8.77 (t, 1H), 8.43 (s, 1H), 8.30-8.00 (m, 3H), 7.68-7.66 (d, 1H), 7.69-7.67 (d, 1H), 7.63-7.60 (m, 2H), 7.39-7.28 (m, 3H), 5.22-5.21 (d, 1H), 4.77-4.75 (m, 1H), 4.64-4.62 (m, 1H), 2.99-2.86 (m, 1H), 2.80-2.46 (m, 4H), 2.17-2.09 (m, 2H), 2.01-1.94 (m, 2H), 1.83-1.77 (m, 1H), 1.01-0.97 (m, 2H), 0.90-0.86 (m, 2H).

LC-MS (ESI): m/z=626.3[M+H]⁺.

### Example 176

### 1-(2-(2-(3-((2-(difluoromethyl)-2H-tetrazol-5-yl)(phenyl)methyl)-3,8-diazabicyclo[3.2.1]octane-8-carbonyl)pyridin-4-yl)benzo[d]oxazol-5-yl)pyrrolidin-2-one

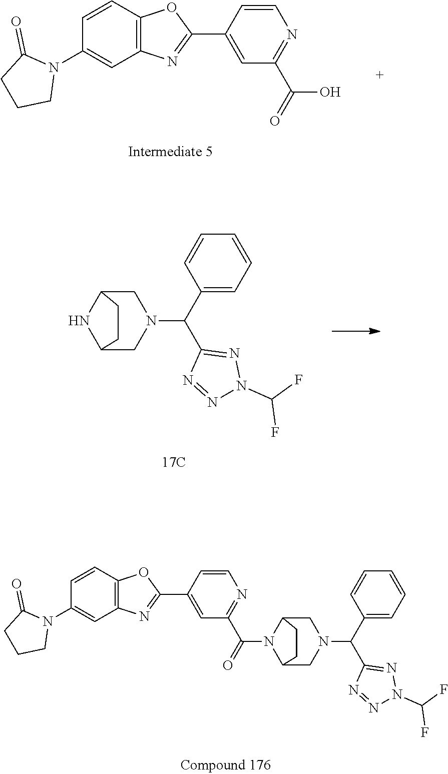 [SMILES: O=C(O)c1cc(-c2nc3cc(N4CCCC4=O)ccc3o2)ccn1 ; FC(F)n1nnc(C(c2ccccc2)N2CC3CCC(C2)N3)n1 ; O=C1CCCN1c1ccc2oc(-c3ccnc(C(=O)N4C5CCC4CN(C(c4ccccc4)c4nnn(C(F)F)n4)C5)c3)nc2c1]

Intermediate 5 (53 mg, 0.18 mmol) was dissolved in N,N-dimethylformamide (2 mL), and the mixture was cooled to 0° C.; HATU (75 mg, 0.2 mmol) and DIPEA (68 mg, 0.525 mmol) were added; and then compound 17C (60 mg, 0.16 mmol) was added. After the addition, the mixture was stirred for 1 hour while the temperature was controlled at 0° C., and then the reaction was quenched by adding ice water (10 mL). The resulting solution was extracted twice with dichloromethane (50 mL×2). The organic phase was combined, dried over anhydrous sodium sulfate and concentrated under reduced pressure, and then the residue was separated by column chromatography (DCM:MeOH=15:1) to obtain compound 34 (20 mg, 20%).

¹H NMR (400 MHz, CDCl₃) δ 8.74 (s, 1H), 8.54 (s, 1H), 8.16 (s, 1H), 7.92 (s, 1H), 7.86-7.81 (m, 1H), 7.70-7.63 (m, 2H), 7.64-7.62 (m, 2H), 7.38-7.30 (m, 3H), 6.13-6.11 (d, 1H), 4.83 (s, 1H), 4.74 (s, 1H), 4.00-3.96 (t, 2H), 3.39 (s, 1H), 2.70-2.66 (m, 4H), 2.51-2.48 (m, 1H), 2.28-2.21 (m, 2H), 2.14-2.12 (m, 2H), 2.00-1.98 (m, 2H).

LC-MS (ESI): m/z=626.3[M+H]⁺.

### Example 177

### (R/S)—N-(5-(2-(4-((2-(difluoromethyl)-2H-tetrazol-5-yl)(phenyl)methyl)piperazine-1-carbonyl)pyridin-4-yl)pyrimidin-2-yl)acetamide (Compound 177)

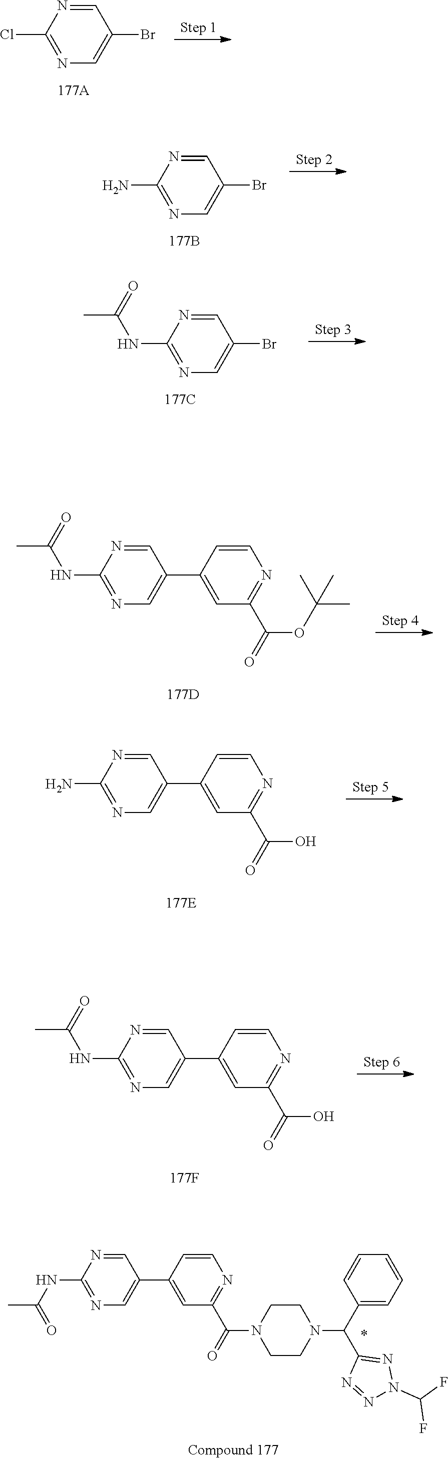

Step 1:

### 5-bromopyrimidin-2-amine (177B)

Aminomethanol solution (4 mol/L, 25 mL) was added to known compound 177A (2.5 g, 12.9 mmol), and the mixture was heated to 100° C. in a sealed tube, stirred for 4 h and concentrated under reduced pressure to obtain 177B (2.1 g, 93%).

LC-MS (ESI): m/z=174.0[M+H]⁺.

Step 2:

### N-(5-bromopyrimidin-2-yl)acetamide (177C)

177B (2.1 g, 12 mmol) was dissolved in glacial acetic acid (21 mL), and then acetic anhydride (3.7 g, 60 mmol) was added. The mixture was stirred at 100° C. for 5 hours. The resulting solution was extracted with water and ethyl acetate, and the ethyl acetate phase was washed once with saturated sodium bicarbonate solution, dried over anhydrous sodium sulfate, concentrated to dryness under reduced pressure and subjected to column chromatography (PE:EA=3:1) to obtain 177C (1.7 g, 65%).

LC-MS (ESI): m/z=216.0[M+H]⁺.

Step 3:

### 4-(2-acetamidopyrimidin-5-yl)picolinate (177D)

177C (400 mg, 1.85 mmol) was dissolved in 1,4-dioxane (8 mL), and then (tert-butyl 4-(4,4,5,5-tetramethyl-1,3,2-dioxin-2-yl)picolinate)(621.2 mg, 2.0 mmol), pd(dppf)Cl₂ (271 mg, 0.37 mmol), sodium carbonate (588 mg, 5.55 mmol) and 2 drops of water were added. Under nitrogen protection, the mixture was warmed to 80° C. and stirred for 7 h. The resulting solution was extracted with water and ethyl acetate; the ethyl acetate phase was dried over anhydrous sodium sulfate and concentrated under reduced pressure; and the residue was separated by column chromatography (PE:EA=2:1) to obtain compound 177D (356 mg, 61.2%).

LC-MS (ESI): m/z=315.2[M+H]⁺.

Step 4:

### 4-(2-aminopyrimidin-5-yl)picolinic Acid (177E)

177D (250 mg, 0.79 mmol) was added to hydrochloric acid/1,4-dioxane (8 mL) solution, and the mixture was stirred at room temperature overnight and concentrated under reduced pressure to obtain 177E (200 mg, 99%).

LC-MS (ESI): m/z=217.0[M+H]⁺.

Step 5:

### 4-(2-acetamidopyrimidin-5-yl)picolinic Acid (177F)

177E (200 mg, 0.9 mmol) was dissolved in glacial acetic acid (4 mL), and then acetic anhydride (283 mg, 5 mmol) was added. The mixture was stirred at 100° C. for 5 hours. The reaction solution was extracted with water and ethyl acetate; the ethyl acetate phase was washed once with saturated sodium bicarbonate solution, dried over anhydrous sodium sulfate and concentrated to dryness under reduced pressure; and the residue was separated and purified by column chromatography (PE:EA=1:2) to obtain 177F (200 mg, 85%).

LC-MS (ESI): m/z=259.0[M+H]⁺.

Step 6:

### (R/S)—N-(5-(2-(4-((2-(difluoromethyl)-2H-tetrazol-5-yl)(phenyl)methyl)piperazine-1-carbonyl)pyridin-4-yl)pyrimidin-2-yl)acetamide (Compound 177)

177F (30 mg, 0.12 mmol) was dissolved in N,N-dimethylformamide (2 mL), and the mixture was cooled to 0° C.; HATU (46 mg, 0.12 mmol) and DIPEA (50 mg, 0.36 mmol) were added; and then intermediate 7 (41 mg, 0.15 mmol) was added. After the addition, the mixture was stirred at room temperature for 2 hours and then extracted twice with water and ethyl acetate. The organic phase was combined, dried over anhydrous sodium sulfate and concentrated under reduced pressure, and then the residue was separated by preparative chromatography to obtain compound 177 (40 mg, 40%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 15.08 min.

¹H NMR (400 MHz, MeOD) δ 9.00 (d, 2H), 8.65 (d, 1H), 8.30-8.01 (t, 1H), 7.91 (s, 1H), 7.81-7.79 (m, 1H), 7.63-7.61 (m, 2H), 7.38-7.31 (m, 3H), 5.18 (m, 1H), 3.83 (s, 2H), 3.55-3.53 (m, 2H), 2.70-2.68 (m, 1H), 2.58-5.56 (m, 2H), 2.43-2.41 (m, 1H), 2.30 (s, 3H).

LC-MS (ESI): m/z=535.2[M+H]⁺.

### Example 178

### (1S,2S)-2-fluoro-N-(2-(2-(4-((R)-(2-Deuteratedmethyl-2H-tetrazol-5-yl)(phenyl)methyl)piperazine-1-carbonyl)pyridin-4-yl)benzo[d]oxazol-5-yl)cyclopropanecarboxamide

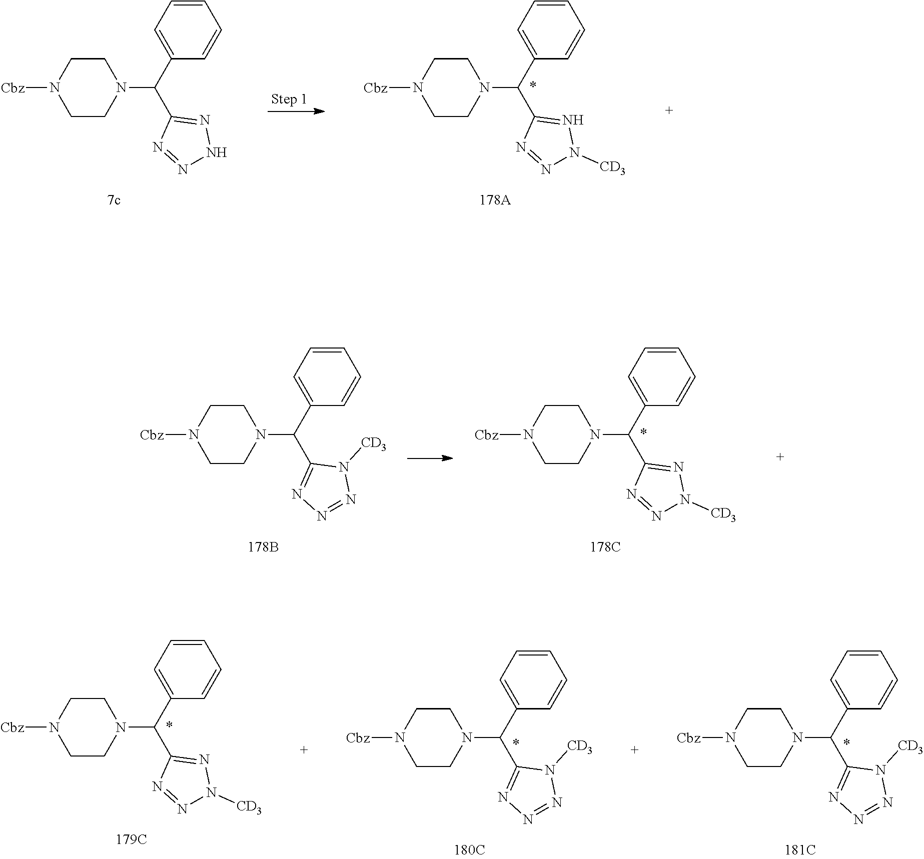

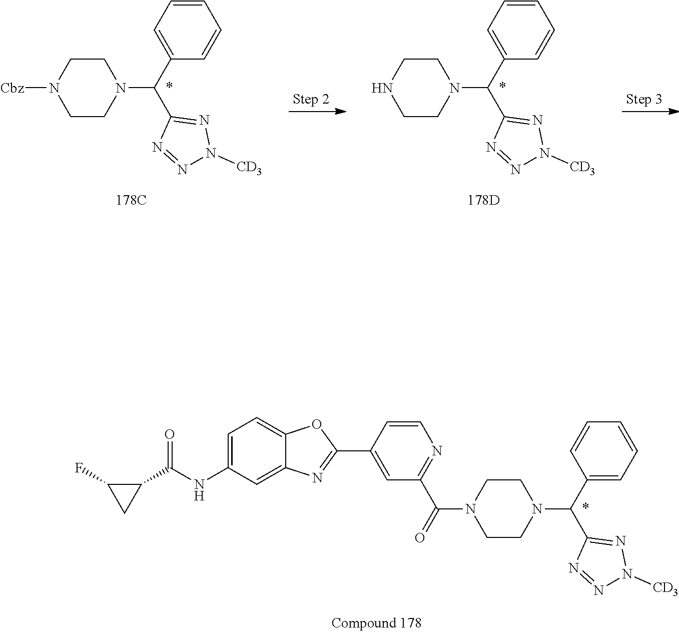 [SMILES: [2H]C([2H])([2H])n1nnc(C(c2ccccc2)N2CCN(C(=O)OCc3ccccc3)CC2)n1 ; [2H]C([2H])([2H])n1nnc(C(c2ccccc2)N2CCNCC2)n1 ; [2H]C([2H])([2H])n1nnc(C(c2ccccc2)N2CCN(C(=O)c3cc(-c4nc5cc(NC(=O)[C@@H]6C[C@@H]6F)ccc5o4)ccn3)CC2)n1]

Step 1:

### (R/S)-benzyl4-((2-Deuteratedmethyl-2H-tetrazol-5-yl)(phenyl)methyl)piperazine-1-carboxylate (178C)

Deuterated iodomethane (5 g, 34 mmol) and benzyl 4-(phenyl(2H-tetrazol-5-yl)methyl)piperazine-1-carboxylate (compound 7c) (9 g, 22.5 mmol) were dissolved in N,N-dimethylformamide (50 mL), and potassium carbonate (6 g, 45 mmol) was added. After the addition, the mixture was stirred at room temperature for 16 hours, and then the reaction was quenched by adding water (200 mL). The reaction solution was extracted with EA. The organic phase was washed with saturated sodium chloride solution, dried over anhydrous sodium sulfate and filtered. The filtrate was concentrated under reduced pressure (EA:PE=1:2) and separated by column chromatography (eluent: PE/EA=30%-50%) to obtain compounds 178A (3.3 g) and 178B (3.5 g). Compound 178A was resolved by chiral HPLC to obtain compound 178C (1.3 g) and compound 179C (1.1 g), with purification conditions as follows: (instrument name: MG II preparative SFC (SFC-1); chromatographic column: ChiralPak AD, 250×30 mm I.D., 10 μm; mobile phase: A for CO₂ and B for ethanol; gradient: B 30%; flow rate: 70 mL/min; column pressure: 100 bar; column temperature: 38° C.; absorption wavelength: 220 nm; cycle time: about 4.5 min). retention time for compound 178C: 5.625 min; retention time for compound 179C: 6.044 min.

Compound 178C: LC-MS (ESI): m/z=396.2 [M+H]⁺.

Compound 179C: LC-MS (ESI): m/z=396.2 [M+H]⁺.

Compound 178B was resolved by chiral HPLC to obtain compound 180C (1.3 g) and compound 181C (1.2 g), with purification conditions as follows:

(instrument name: MG II preparative SFC (SFC-1); chromatographic column: Cellulose-2, 250×30 mm I.D., 5 μm; mobile phase: A for CO₂ and B for methanol; gradient: B 40%; flow rate: 60 mL/min; column pressure: 100 bar; column temperature: 38° C.; absorption wavelength: 220 nm; cycle time: about 6.3 min). retention time for compound 180C: 1.415 min; retention time for compound 181C: 1.709 min.

Compound 180C: LC-MS (ESI): m/z=396.2 [M+H]⁺.

Compound 181C: LC-MS (ESI): m/z=396.2 [M+H]⁺.

Step 2:

### (R/S)-1-((2-Deuteratedmethyl-2H-tetrazol-5-yl)(phenyl)methyl)piperazine (178D)

Compound 178C (1.3 g, 3.29 mmol) and Pd/C (260 mg) were added to methanol (10 mL). Under hydrogen replacement, the mixture was reacted at room temperature for 2 h and filtered. The filtrate was concentrated under reduced pressure to obtain 178D (835 mg, 98%).

Step 3:

### (1S,2S)-2-fluoro-N-(2-(2-(4-((R)-(2-Deuteratedmethyl-2H-tetrazol-5-yl)(phenyl)methyl)piperazine-1-carbonyl)pyridin-4-yl)benzo[d]oxazol-5-yl)cyclopropanecarboxamide (Compound 178)

117F (200 mg, 0.59 mmol) and 178D (184 mg, 0.7 mmol) were dissolved in N,N-dimethylformamide (5 mL). HATU (336 mg, 0.88 mmol) and DIPEA (228 mg, 1.77 mmol) were added. After the addition, the mixture was stirred for 1 hour at room temperature, and then the reaction was quenched by adding water (30 mL). The resulting solution was extracted twice with ethyl acetate (30 mL×3). The organic phase was combined, dried over anhydrous sodium sulfate and concentrated under reduced pressure, and then the residue was separated by column chromatography (eluent: MeOH/DCM=1/20) to obtain compound 178 (100 mg, 29%).

¹H NMR (400 MHz, MeOD) δ 8.76-8.73 (m, 1H), 8.25-8.24 (m, 1H), 8.15-8.10 (m, 2H), 7.62 (d, 1H), 7.58-7.48 (m, 3H), 7.35-7.24 (m, 3H), 5.01 (s, 1H), 4.95-4.91 (m, 1H), 4.79-4.75 (m, 1H), 3.83-3.81 (m, 2H), 3.56 (t, 2H), 2.72-2.60 (m, 1H), 2.61-2.46 (m, 2H), 2.46-2.35 (m, 1H), 2.03-1.96 (m, 1H), 1.83-1.73 (m, 1H), 1.32-1.13 (m, 1H).

LC-MS (ESI): m/z=585.2[M+H]⁺.

### Example 179

### (1S,2S)-2-fluoro-N-(2-(2-(4-((S)-(2-Deuteratedmethyl-2H-tetrazol-5-yl)(phenyl)methyl)piperazine-1-carbonyl)pyridin-4-yl)benzo[d]oxazol-5-yl)cyclopropanecarboxamide

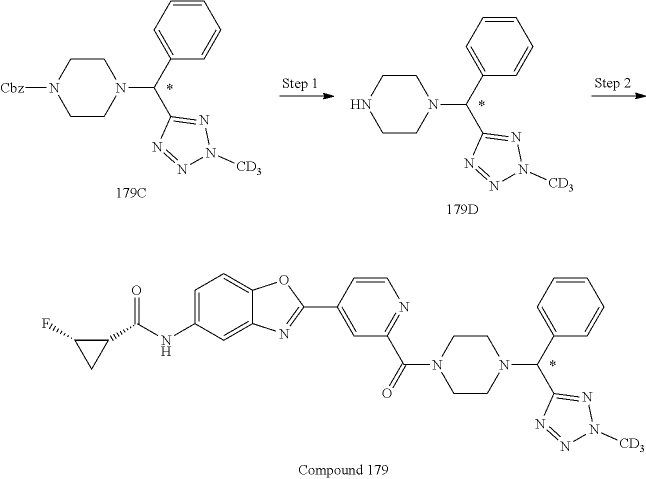

Step 1:

### (S/R)-1-((2-Deuteratedmethyl-2H-tetrazol-5-yl)(phenyl)methyl)piperazine (179D)

Compound 179C (1.1 g, 2.78 mmol) and Pd/C (260 mg) were added to methanol (10 mL). Under hydrogen replacement, the mixture was reacted at room temperature for 2 h and filtered. The filtrate was concentrated under reduced pressure to obtain 179D (691 mg, 95%).

Step 2:

### (1S,2S)-2-fluoro-N-(2-(2-(4-((S/R)-(2-Deuteratedmethyl-2H-tetrazol-5-yl)(phenyl)methyl)piperazine-1-carbonyl)pyridin-4-yl)benzo[d]oxazol-5-yl)cyclopropanecarboxamide (Compound 179)

117F (200 mg, 0.59 mmol) and 179D (184 mg, 0.7 mmol) were dissolved in N,N-dimethylformamide (5 mL). HATU (336 mg, 0.88 mmol) and DIPEA (228 mg, 1.77 mmol) were added. After the addition, the mixture was stirred for 1 hour at room temperature, and then the reaction was quenched by adding water (30 mL). The resulting solution was extracted twice with ethyl acetate (30 mL×3). The organic phase was combined, dried over anhydrous sodium sulfate and concentrated under reduced pressure, and then the residue was separated by column chromatography (eluent: MeOH/DCM=1/20) to obtain compound 162 (100 mg, 29%).

¹H NMR (400 MHz, MeOD) δ 8.79-8.74 (m, 1H), 8.27 (s, 1H), 8.17-8.15 (m, 2H), 7.66-7.64 (m, 1H), 7.59-7.56 (m, 1H), 7.55-7.48 (m, 2H), 7.37-7.25 (m, 3H), 5.01 (s, 1H), 4.94-4.92 (m, 1H), 4.78-4.74 (m, 1H), 3.84-3.82 (m, 2H), 3.56 (t, 2H), 2.70-2.61 (m, 1H), 2.60-2.48 (m, 2H), 2.47-2.36 (m, 1H), 2.05-1.98 (m, 1H), 1.83-1.73 (m, 1H), 1.25-1.11 (m, 1H).

LC-MS (ESI): m/z=585.2[M+H]⁺.

### Example 180

### (1S,2S)-2-fluoro-N-(2-(2-(4-((R/S)-(1-Deuteratedmethyl-1H-tetrazol-5-yl)(phenyl)methyl)piperazine-1-carbonyl)pyridin-4-yl)benzo[d]oxazol-5-yl)cyclopropanecarboxamide

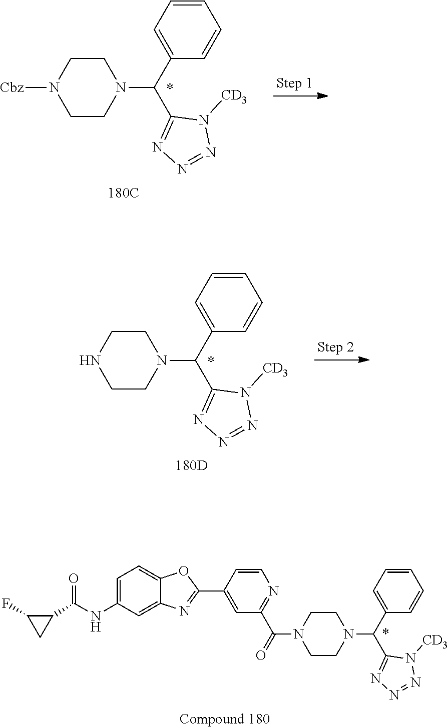

Step 1:

### (R/S)-1-((1-Deuteratedmethyl-1H-tetrazol-5-yl)(phenyl)methyl)piperazine (180D)

Compound 180C (1.3 g, 3.29 mmol) and Pd/C (260 mg) were added to methanol (10 mL). Under hydrogen replacement, the mixture was reacted at room temperature for 2 h and filtered. The filtrate was concentrated under reduced pressure to obtain 180D (800 mg, 93%).

Step 2:

### (1S,2S)-2-fluoro-N-(2-(2-(4-((R/S)-(1-Deuteratedmethyl-1H-tetrazol-5-yl)(phenyl)methyl)piperazine-1-carbonyl)pyridin-4-yl)benzo[d]oxazol-5-yl)cyclopropanecarboxamide (Compound 180)

117F (200 mg, 0.59 mmol) and 180D (184 mg, 0.7 mmol) were dissolved in N,N-dimethylformamide (5 mL). HATU (336 mg, 0.88 mmol) and DIPEA (228 mg, 1.77 mmol) were added. After the addition, the mixture was stirred for 1 hour at room temperature, and then the reaction was quenched by adding water (30 mL). The resulting solution was extracted twice with ethyl acetate (30 mL×3). The organic phase was combined, dried over anhydrous sodium sulfate and concentrated under reduced pressure, and then the residue was separated by column chromatography (eluent: MeOH/DCM=1/20) to obtain compound 180 (110 mg, 32%).

¹H NMR (400 MHz, DMSO) δ 10.47 (s, 1H), 8.82-8.80 (m, 1H), 8.23 (d, 1H), 8.18-8.16 (m, 1H), 8.14-8.13 (m, 1H), 7.79 (d, 1H), 7.62 (dd, 1H), 7.48-7.46 (m, 2H), 7.44-7.32 (m, 3H), 5.40 (s, 1H), 5.06-4.85 (m, 1H), 3.72-3.70 (m, 2H), 3.50-3.48 (m, 2H), 2.69-2.58 (m, 1H), 2.50-2.48 (m, 2H), 2.41-2.33 (m, 1H), 2.09-2.01 (m, 1H), 1.73-1.62 (m, 1H), 1.23-1.13 (m, 1H).

LC-MS (ESI): m/z=585.2[M+H]⁺.

### Example 181

### (1S,2S)-2-fluoro-N-(2-(2-(4-((S/R)-(1-Deuteratedmethyl-1H-tetrazol-5-yl)(phenyl)methyl)piperazine-1-carbonyl)pyridin-4-yl)benzo[d]oxazol-5-yl)cyclopropanecarboxamide

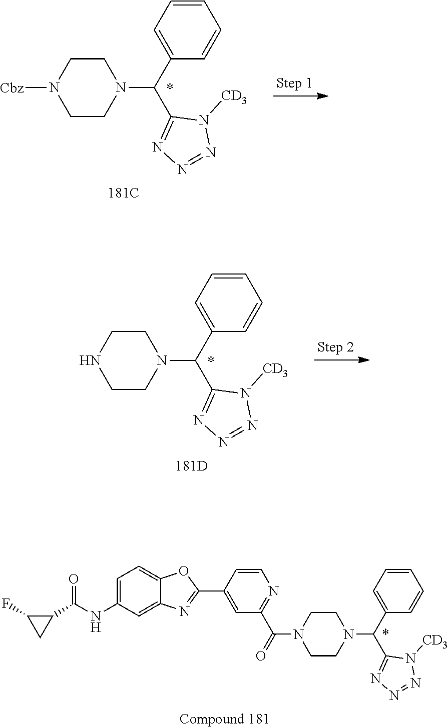

Step 1:

### (S/R)-1-((1-Deuteratedmethyl-1H-tetrazol-5-yl)(phenyl)methyl)piperazine (181D)

Compound 181C (1.2 g, 3.04 mmol) and Pd/C (260 mg) were added to methanol (10 mL). Under hydrogen replacement, the mixture was reacted at room temperature for 2 h and filtered. The filtrate was concentrated under reduced pressure to obtain 181D (757 mg, 95%).

Step 2:

### (1S,2S)-2-fluoro-N-(2-(2-(4-((S/R)-(1-Deuteratedmethyl-1H-tetrazol-5-yl)(phenyl)methyl)piperazine-1-carbonyl)pyridin-4-yl)benzo[d]oxazol-5-yl)cyclopropanecarboxamide (Compound 181)

117F (200 mg, 0.59 mmol) and 181D (184 mg, 0.7 mmol) were dissolved in N,N-dimethylformamide (5 mL). HATU (336 mg, 0.88 mmol) and DIPEA (228 mg, 1.77 mmol) were added. After the addition, the mixture was stirred for 1 hour at room temperature, and then the reaction was quenched by adding water (30 mL). The resulting solution was extracted twice with ethyl acetate (30 mL×3). The organic phase was combined, dried over anhydrous sodium sulfate and concentrated under reduced pressure, and then the residue was separated by column chromatography (eluent: MeOH/DCM=1/20) to obtain compound 181 (110 mg, 32%).

¹H NMR (400 MHz, DMSO) δ 10.47 (s, 1H), 8.81-8.79 (m, 1H), 8.24 (d, 1H), 8.18-8.16 (m, 1H), 8.14-8.13 (m, 1H), 7.79 (d, 1H), 7.63 (dd, 1H), 7.49-7.44 (m, 2H), 7.44-7.33 (m, 3H), 5.40 (s, 1H), 5.06-4.85 (m, 1H), 3.73-7.31 (m, 2H), 3.49 (d, 2H), 2.69-2.59 (m, 1H), 2.51-2.49 (m, 2H), 2.43-2.34 (m, 1H), 2.08-2.01 (m, 1H), 1.73-1.63 (m, 1H), 1.21-1.13 (m, 1H).

LC-MS (ESI): m/z=585.2[M+H]⁺.

### Example 182

### (R/S)-(4-(5-(1-methyl-1H-pyrazol-5-yl)benzo[d]oxazol-2-yl)pyridin-2-yl)(4-((5-methyl-2H-tetrazol-2-yl)(phenyl)methyl)piperidin-1-yl)methanone

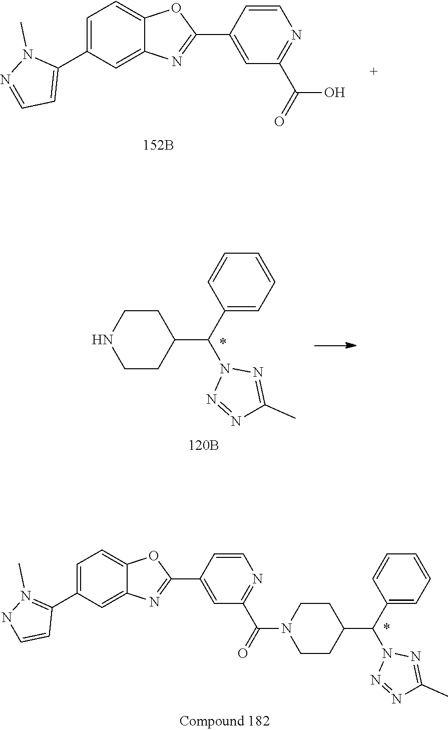

Compounds 152B and 120B were used as raw materials to obtain compound 182 according to the synthetic method of compound 1.

LC-MS (ESI): m/z=560.3 [M+H]⁺.

¹H NMR (400 MHz, CDCl₃) δ 8.79-8.76 (m, 1H), 8.44 (s, 1H), 8.15-8.13 (m, 1H), 7.89 (s, 1H), 7.76-7.74 (m, 1H), 7.70-7.69 (m, 1H), 7.55-7.50 (m, 3H), 7.40-7.34 (m, 3H), 6.48-6.47 (m, 1H), 5.54-5.51 (m, 1H), 4.78-4.76 (m, 1H), 4.05-4.00 (m, 3H), 3.13-3.09 (m, 1H), 2.90-2.82 (m, 2H), 2.55-2.50 (m, 3H), 1.60-1.23 (m, 5H).

### Example 183

### (R/S)-(4-(5-(1-methyl-1H-pyrazol-3-yl)benzo[d]oxazol-2-yl)pyridin-2-yl)(4-((5-methyl-2H-tetrazol-2-yl)(phenyl)methyl)piperidin-1-yl)methanone

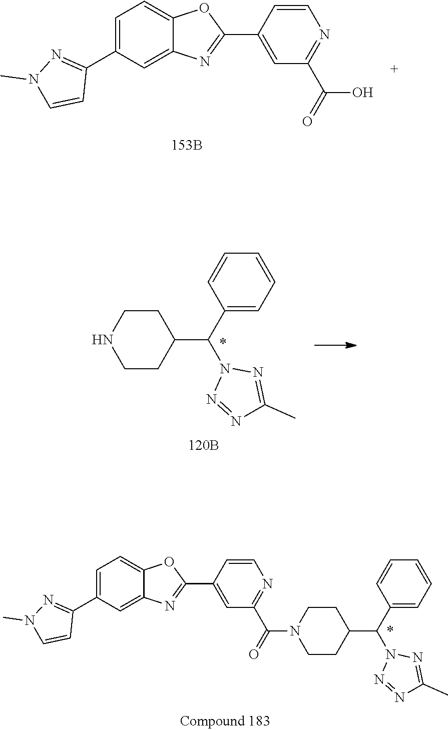

Compounds 153B and 120B were used as raw materials to obtain compound 183 according to the synthetic method of compound 1.

LC-MS (ESI): m/z=560.3 [M+H]⁺.

¹H NMR (400 MHz, CDCl₃) δ 8.76-8.74 (m, 1H), 8.42 (s, 1H), 8.24-8.23 (m, 1H), 8.16-8.14 (m, 1H), 8.10-8.07 (m, 1H), 7.69-7.66 (m, 1H), 7.55-7.48 (m, 3H), 7.40-7.33 (m, 3H), 6.67-6.66 (m, 1H), 5.54-5.51 (m, 1H), 4.78-4.76 (m, 1H), 4.10 (s, 3H), 3.15-3.11 (m, 1H), 2.90-2.82 (m, 2H), 2.55-2.50 (m, 3H), 1.56-1.23 (m, 5H).

### Example 184

### (1R,2S)-2-fluoro-N-(2-(2-(4-((R/S)-(5-methyl-2H-tetrazol-2-yl)(phenyl)methyl)piperidine-1-carbonyl)pyridin-4-yl)benzo[d]oxazol-5-yl)cyclopropanecarboxamide

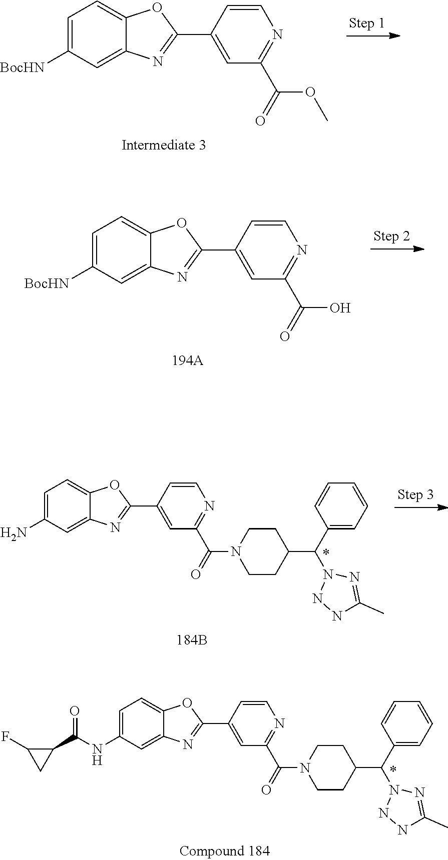 [SMILES: COC(=O)c1cc(-c2nc3cc(NC(=O)OC(C)(C)C)ccc3o2)ccn1 ; CC(C)(C)OC(=O)Nc1ccc2oc(-c3ccnc(C(=O)O)c3)nc2c1 ; CC1=NN(C(c2ccccc2)C2CCN(C(=O)c3cc(-c4nc5cc(N)ccc5o4)ccn3)CC2)NN1 ; CC1=NN(C(c2ccccc2)C2CCN(C(=O)c3cc(-c4nc5cc(NC(=O)[C@H]6CC6F)ccc5o4)ccn3)CC2)NN1]

Step 1:

### 4-(5-((tert-butoxycarbonyl)amino)benzo[d]oxazol-2-yl)picolinic Acid (184A)

Intermediate 3 (2.0 g, 5.4 mmol) was dissolved in methanol (20 mL) at room temperature, and then sodium hydroxide (432 mg, 10.8 mmol) was added. The mixture was stirred at room temperature for 30 min. The resulting solution was adjusted to pH 5-6 by adding 2 M dilute hydrochloric acid and concentrated under reduced pressure to remove methanol. Water (20 mL) was added, and the resulting solution was extracted with DCM (10 mL×3). The organic phase was washed with saturated sodium chloride solution (10 mL×1), dried over anhydrous sodium sulfate, filtered and concentrated to obtain compound 184A (1.8 g, yield: 94.7%).

LC-MS (ESI): m/z=356.2[M+H]⁺.

Step 2:

### (R/S)-(4-(5-aminobenzo[d]oxazol-2-yl)pyridin-2-yl)(4-((5-methyl-2H-tetrazol-2-yl)(phenyl)methyl)piperidin-1-yl)methanone (184B)

Compound 184A (1.8 g, 5.1 mmol) was added to DCM (20 mL) at room temperature; 120B (1.3 g, 5.1 mmol) was added; and then DIPEA (1.9 g, 15.3 mmol) and HATU (2.3 g, 6.1 mmol) were successively added. The mixture was stirred at room temperature for 30 min. Water (20 mL) was added, and the resulting solution was extracted with DCM (10 mL×3). The organic phase was washed with saturated sodium chloride solution (10 mL×1), dried over anhydrous sodium sulfate, filtered, concentrated and then added to DCM (20 mL). Then trifluoroacetic acid (5 mL) was added, and the mixture was stirred at room temperature for 2 h. The mixture was concentrated and then adjusted to pH 8-9 by adding saturated sodium bicarbonate solution. Water (20 mL) was added, and the solution was extracted with DCM (10 mL×3). The organic phase was washed with saturated sodium chloride solution (10 mL×1), dried over anhydrous sodium sulfate, filtered and concentrated, and then the residue was subjected to column chromatography (DCM:MeOH=40:1 to 10:1) to obtain compound 184B (1.2 g, yield: 48%).

LC-MS (ESI): m/z=495.2[M+H]⁺.

Step 3:

### (1R,2S)-2-fluoro-N-(2-(2-(4-((R/S)-(5-methyl-2H-tetrazol-2-yl)(phenyl)methyl)piperidine-1-carbonyl)pyridin-4-yl)benzo[d]oxazol-5-yl)cyclopropanecarboxamide (Compound 184)

Compound 184B (0.2 g, 0.4 mmol) was added to DCM (10 mL) at room temperature; (1R,2S)-2-fluorocyclopropanecarboxylic acid (41.6 mg, 0.4 mmol) was added; and then DIPEA (154 mg, 1.2 mmol) and HATU (182 mg, 0.48 mmol) were successively added. The mixture was stirred at room temperature for 30 min. Water (20 mL) was added, and the solution was extracted with DCM (10 mL×3). The organic phase was washed with saturated sodium chloride solution (10 mL×1), dried over anhydrous sodium sulfate, filtered and concentrated, and the residue was subjected to column chromatography (DCM:MeOH=40:1 to 20:1) to obtain compound 184 (0.1 g, yield: 24%).

LC-MS (ESI): m/z=581.2[M+H]⁺.

¹H NMR (400 MHz, CDCl3) δ 8.72-8.71 (m, 1H), 8.36-8.34 (m, 1H), 8.22 (s, 1H), 8.06-8.04 (m, 1H), 7.89 (s, 1H), 7.55-7.50 (m, 2H), 7.46-7.31 (m, 5H), 5.54-5.51 (m, 1H), 4.98-4.75 (m, 2H), 3.91-3.87 (m, 1H), 3.15-3.11 (m, 1H), 2.96-2.84 (m, 2H), 2.55-2.49 (m, 3H), 1.60-1.23 (m, 7H).

### Example 185

### (1R,2R)-2-fluoro-N-(2-(2-(4-((R/S)-(5-methyl-2H-tetrazol-2-yl)(phenyl)methyl)piperidine-1-carbonyl)pyridin-4-yl)benzo[d]oxazol-5-yl)cyclopropanecarboxamide

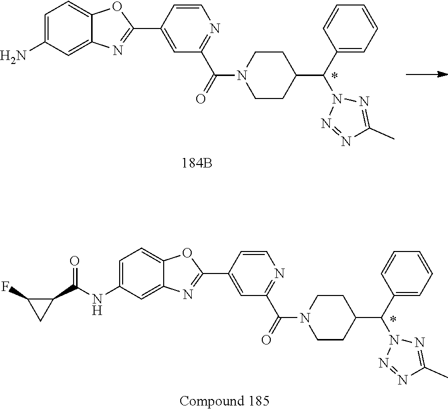 [SMILES: Cc1nnn(C(c2ccccc2)C2CCN(C(=O)c3cc(-c4nc5cc(N)ccc5o4)ccn3)CC2)n1 ; Cc1nnn(C(c2ccccc2)C2CCN(C(=O)c3cc(-c4nc5cc(NC(=O)[C@H]6C[C@H]6F)ccc5o4)ccn3)CC2)n1]

Compounds 184B and (1R,2R)-2-fluorocyclopropanecarboxylic acid were used as raw materials to obtain compound 185 according to the synthetic method of compound 184.

LC-MS (ESI): m/z=581.3[M+H]⁺.

¹H NMR (400 MHz, CDCl₃) δ 8.73-8.71 (m, 1H), 8.26-8.25 (m, 1H), 8.07-7.97 (m, 3H), 7.55-7.31 (m, 7H), 5.54-5.51 (m, 1H), 4.91-4.87 (m, 0.5H), 4.79-4.72 (m, 1.5H), 3.92-3.88 (m, 1H), 3.16-3.07 (m, 1H), 2.94-2.79 (m, 2H), 2.55-2.49 (m, 3H), 1.90-1.82 (m, 2H), 1.46-1.23 (m, 5H).

### Example 186

### (R/S)—N-(2-(2-(4-((5-(difluoromethyl)-2H-tetrazol-2-yl)(phenyl)methyl)piperidine-1-carbonyl)pyridin-4-yl)benzo[d]oxazol-5-yl)-2-fluoro-2-methylpropanamide

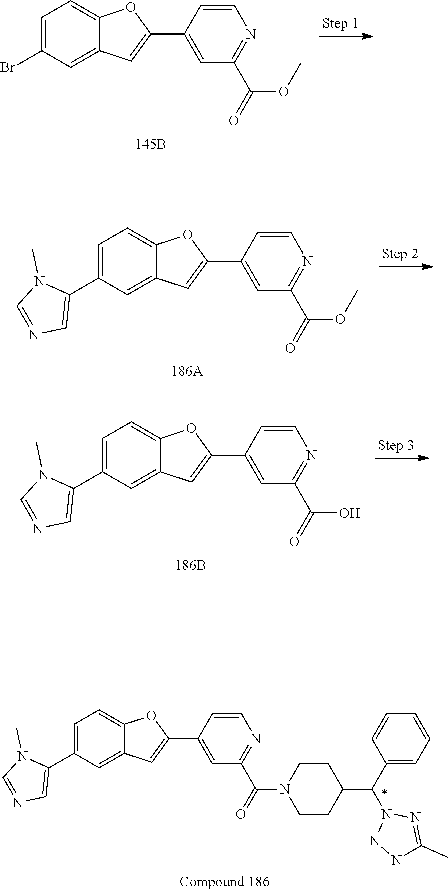 [SMILES: COC(=O)c1cc(-c2cc3cc(Br)ccc3o2)ccn1 ; COC(=O)c1cc(-c2cc3cc(-c4cncn4C)ccc3o2)ccn1 ; Cn1cncc1-c1ccc2oc(-c3ccnc(C(=O)O)c3)cc2c1 ; CC1=NN(C(c2ccccc2)C2CCN(C(=O)c3cc(-c4cc5cc(-c6cncn6C)ccc5o4)ccn3)CC2)NN1]

Step 1:

### Methyl 4-(5-(1-methyl-1H-imidazol-5-yl)benzofuran-2-yl)picolinate (186A)

Under nitrogen protection, 145B (200 mg, 0.17 mmol), 1,4-dioxane (9 mL), water (1 mL), 1-methyl-5-(4,4,5,5-tetramethyl-1,3,2-dioxin-2-yl)-1H imidazole (150 mg, 0.72 mmol), Cs₂CO₃ (326 mg, 1 mmol) and PdCl₂(dppf) (73 mg, 0.1 mmol) were successively added to a single-necked flask, and the mixture was stirred at 100° C. for 3 h. The reaction was cooled to room temperature and concentrated under reduced pressure to obtain a crude product from the residue, and the crude product was separated by preparative column chromatography (eluent: EA/PE=1/2) to obtain compound 186A as a white solid (150 mg, 74.7%).

L C-MS (ESI): m/z=334.2 [M+H]⁺.

Step 2:

### 4-(5-(1-methyl-1H-imidazol-5-yl)benzofuran-2-yl)picolinic Acid (186B)

At room temperature, compound 186A (0.15 g, 0.5 mmol) was dissolved in methanol (15 mL), and lithium hydroxide (50 mg) was dissolved in 20 mL of pure water. An aqueous solution of lithium hydroxide was added to the reaction solution; the mixture was stirred at 40° C. for 0.5 hours and then adjusted to pH 6-7 with 2 N hydrochloric acid; and the resulting solution was extracted with ethyl acetate (30 mL×3). The combined organic phase was dried over anhydrous sodium sulfate and concentrated under reduced pressure to obtain 186B as a white solid (0.12 g, 83.15%).

LC-MS (ESI): m/z=320.2 [M+H]⁺.

Step 3:

### (R/S)-(4-(5-(1-methyl-1H-imidazol-5-yl)benzofuran-2-yl)pyridin-2-yl)(4-((5-methyl-2H-tetrazol-2-yl)(phenyl)methyl)piperidin-1-yl)methanone (Compound 186)

Under nitrogen protection, compound 186B (60 mg, 0.18 mmol), DMF (5 mL), HATU (77.5 mg, 0.20 mmol), DIPEA (33 mg, 0.25 mmol) and 120B (61 mg, 0.21 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 186 as a light yellow solid (30 mg, 28.58%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.08 min.

¹H NMR (400 MHz, CDCl₃) δ 8.65-8.62 (m, 1H), 7.75-7.74 (m, 1H), 7.66-7.63 (m, 2H), 7.55-7.51 (m, 2H), 7.39-7.26 (m, 4H), 7.21-6.19 (m, 1H), 5.53-5.51 (m, 1H), 4.80-4.76 (m, 1H), 4.09-4.05 (m, 1H), 3.79 (s, 3H), 3.11-3.07 (m, 1H), 2.88-2.80 (m, 2H), 2.55-2.50 (m, 3H), 1.45-1.23 (m, 6H).

LC-MS (ESI): m/z=559.2 [M+H]⁺.

### Example 187

### (R/S)-(4-(5-(1,5-dimethyl-1H-pyrazol-4-yl)benzo[d]oxazol-2-yl)pyridin-2-yl)(4-((5-methyl-2H-tetrazol-2-yl)(phenyl)methyl)piperidin-1-yl)methanone

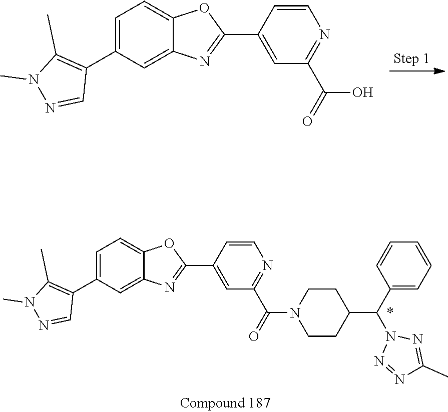

Step 1:

### (R/S)-(4-(5-(1,5-dimethyl-1H-pyrazol-4-yl)benzo[d]oxazol-2-yl)pyridin-2-yl)(4-((5-methyl-2H-tetrazol-2-yl)(phenyl)methyl)piperidin-1-yl)methanone (Compound 187)

Under nitrogen protection, 154B (60 mg, 0.18 mmol), DMF (5 mL), HATU (77.5 mg, 0.20 mmol), DIPEA (33 mg, 0.25 mmol) and 120B (55 mg, 0.21 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 187 as a light yellow solid (40 mg, 38.8%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.45 min.

¹H NMR (400 MHz, CDCl₃) δ 8.77-8.74 (m, 1H), 8.42 (s, 1H), 8.13 (s, 1H), 7.77-7.74 (m, 2H), 7.69-7.67 (m, 1H), 7.55-7.41 (m, 2H), 7.38-7.21 (m, 3H), 5.54-5.51 (m, 1H), 4.80-4.76 (m, 1H), 4.05 (s, 3H), 4.01-3.98 (m, 1H), 3.12-3.09 (m, 1H), 2.95-2.78 (m, 2H), 2.55-2.50 (m, 2H), 2.47 (s, 3H), 1.60-1.23 (m, 5H).

LC-MS (ESI): m/z=574.3 [M+H]⁺.

### Example 188

### (R/S)-(4-(5-(3-methyl-1H-pyrazol-4-yl)benzo[d]oxazol-2-yl)pyridin-2-yl)(4-((5-methyl-2H-tetrazol-2-yl)(phenyl)methyl)piperidin-1-yl)methanone

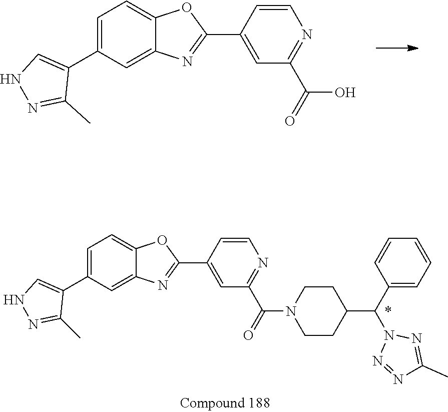 [SMILES: Cc1n[nH]cc1-c1ccc2oc(-c3ccnc(C(=O)O)c3)nc2c1 ; Cc1nnn(C(c2ccccc2)C2CCN(C(=O)c3cc(-c4nc5cc(-c6c[nH]nc6C)ccc5o4)ccn3)CC2)n1]

Step 1:

### (R/S)-(4-(5-(3-methyl-1H-pyrazol-4-yl)benzo[d]oxazol-2-yl)pyridin-2-yl)(4-((5-methyl-2H-tetrazol-2-yl)(phenyl)methyl)piperidin-1-yl)methanone (Compound 188)

Under nitrogen protection, 155B (90 mg, 0.28 mmol), DMF (5 mL), HATU (114 mg, 0.30 mmol), DIPEA (50 mg, 0.5 mmol) and 120B (72 mg, 0.28 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 188 as a light yellow solid (21 mg, 13.3%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 11.05 min.

¹H NMR (400 MHz, CDCl₃) δ 8.76-8.74 (m, 1H), 8.41 (s, 1H), 8.12 (s, 1H), 7.83-7.79 (m, 2H), 7.68-7.66 (m, 1H), 7.55-7.41 (m, 3H), 7.38-7.30 (m, 3H), 5.54-5.51 (m, 1H), 4.80-4.76 (m, 1H), 4.07-4.02 (m, 1H), 3.12-3.08 (m, 1H), 2.95-2.78 (m, 2H), 2.55 (s, 3H), 2.51-2.50 (m, 1H), 2.47 (s, 2H), 1.60-1.26 (m, 5H).

LC-MS (ESI): m/z=560.3 [M+H]⁺.

### Example 189

### (R/S)-(4-(5-(4-methyl-1H-imidazol-1-yl)benzo[d]oxazol-2-yl)pyridin-2-yl)(4-((5-methyl-2H-tetrazol-2-yl)(phenyl)methyl)piperidin-1-yl)methanone

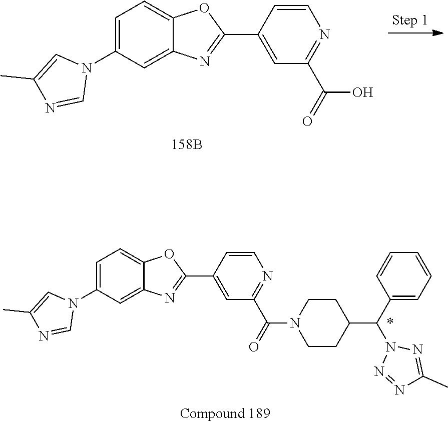

Step 1:

### (R/S)-(4-(5-(4-methyl-1H-imidazol-1-yl)benzo[d]oxazol-2-yl)pyridin-2-yl)(4-((5-methyl-2H-tetrazol-2-yl)(phenyl)methyl)piperidin-1-yl)methanone (Compound 189)

Under nitrogen protection, 158B (50 mg, 0.15 mmol), DMF (5 mL), HATU (64.6 mg, 0.17 mmol), DIPEA (33 mg, 0.25 mmol) and 120B (40 mg, 0.19 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 189 as a light yellow solid (30 mg, 34.3%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.70 min.

¹H NMR (400 MHz, CDCl₃) δ 8.79-8.76 (m, 1H), 8.42 (s, 1H), 8.25 (s, 1H), 8.13 (s, 1H), 7.85-7.76 (m, 2H), 7.55-7.50 (m, 3H), 7.42-7.34 (m, 3H), 7.12 (s, 1H), 5.54-5.51 (m, 1H), 4.80-4.76 (m, 1H), 4.01-3.98 (m, 1H), 3.12-3.09 (m, 1H), 2.95-2.78 (m, 2H), 2.55-2.50 (m, 2H), 2.45 (s, 3H), 1.60-1.24 (m, 5H).

LC-MS (ESI): m/z=560.3 [M+H]⁺.

### Example 190

### (1S,2R)-2-fluoro-N-(2-(2-(4-((R/S)-(5-methyl-2H-tetrazol-2-yl)(phenyl)methyl)piperidine-1-carbonyl)pyridin-4-yl)benzo[d]oxazol-5-yl)cyclopropane-1-carboxamide

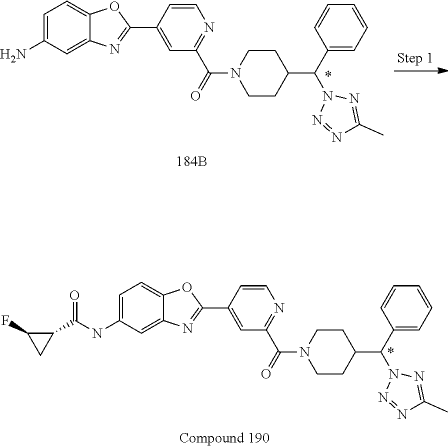 [SMILES: Cc1nnn(C(c2ccccc2)C2CCN(C(=O)c3cc(-c4nc5cc(N)ccc5o4)ccn3)CC2)n1 ; Cc1nnn(C(c2ccccc2)C2CCN(C(=O)c3cc(-c4nc5cc(NC(=O)[C@@H]6C[C@H]6F)ccc5o4)ccn3)CC2)n1]

Step 1:

### (1S,2R)-2-fluoro-N-(2-(2-(4-((R/S)-(5-methyl-2H-tetrazol-2-yl)(phenyl)methyl)piperidine-1-carbonyl)pyridin-4-yl)benzo[d]oxazol-5-yl)cyclopropane-1-carboxamide (Compound 190)

Under nitrogen protection, compound (1S, 2R)-2-fluorocyclopropanecarboxylic acid (42 mg, 0.4 mmol), DMF (5 mL), HATU (182 mg, 0.4 mmol), DIPEA (154 mg, 1.2 mmol) and 184B (200 mg, 0.4 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 190 as a white solid (80 mg, 34.3%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.19 min.

¹H NMR (400 MHz, CDCl3) δ 8.73-8.70 (m, 1H), 8.31-8.22 (m, 2H), 8.05-8.02 (m, 1H), 7.89 (s, 1H), 7.55-7.31 (m, 7H), 5.54-5.51 (m, 1H), 4.97-4.96 (m, 0.5H), 4.80-4.75 (m, 1.5H), 3.94-3.91 (m, 1H), 3.16-3.09 (m, 1H), 2.96-2.87 (m, 2H), 2.55-2.50 (m, 3H), 1.61-1.23 (m, 7H).

LC-MS (ESI): m/z=581.3 [M+H]⁺.

### Example 191

### (3S)—N-(2-(2-(4-((2-methyl-2H-tetrazol-5-yl)(phenyl)methyl)piperazine-1-carbonyl)pyridin-4-yl)benzo[d]oxazol-5-yl)tetrahydrofuran-3-carboxamide (Compound 191)

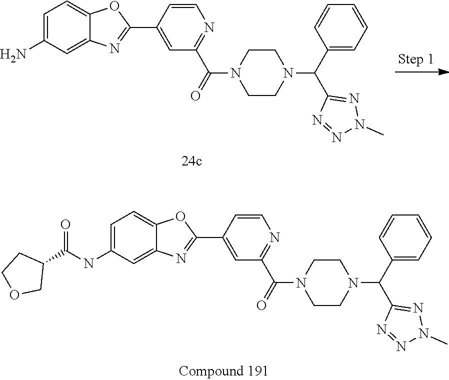 [SMILES: Cn1nnc(C(c2ccccc2)N2CCN(C(=O)c3cc(-c4nc5cc(N)ccc5o4)ccn3)CC2)n1 ; Cn1nnc(C(c2ccccc2)N2CCN(C(=O)c3cc(-c4nc5cc(NC(=O)[C@H]6CCOC6)ccc5o4)ccn3)CC2)n1]

Step 1:

### (3S)—N-(2-(2-(4-((2-methyl-2H-tetrazol-5-yl)(phenyl)methyl)piperazine-1-carbonyl)pyridin-4-yl)benzo[d]oxazol-5-yl)tetrahydrofuran-3-carboxamide (Compound 191)

At room temperature, DMF (5 mL), (3S)-tetrahydrofuran-3-carboxylic acid (56 mg, 0.48 mmol), HATU (183 mg, 0.48 mmol), and TEA (122 mg, 1.2 mmol) were successively added to compound 24C (199 mg, 0.41 mmol). The reaction was stirred for 1 hour. The reaction solution was poured into water (50 mL) and extracted with ethyl acetate (50 mL×2). The organic phase was concentrated, and the residue was separated and purified by HPLC to obtain compound 191 (48 mg, 21%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.33 min.

LC-MS (ESI): m/z=594.3 [M+H]⁺.

¹H NMR (400 MHz, CDCl₃) δ 8.79-8.62 (m, 1H), 8.21 (s, 2H), 8.07-7.96 (m, 1H), 7.92 (s, 1H), 7.55-7.49 (m, 2H), 7.49-7.39 (m, 2H), 7.38-7.28 (m, 3H), 5.01 (s, 1H), 4.34 (s, 3H), 4.11-3.96 (m, 3H), 3.96-3.79 (m, 3H), 3.63 (s, 2H), 3.17-3.01 (m, 1H), 2.76-2.64 (m, 1H), 2.64-2.50 (m, 2H), 2.48-2.37 (m, 1H), 2.36-2.18 (m, 2H).

### Example 192

### (3R)—N-(2-(2-(4-((2-methyl-2H-tetrazol-5-yl)(phenyl)methyl)piperazine-1-carbonyl)pyridin-4-yl)benzo[d]oxazol-5-yl)tetrahydrofuran-3-carboxamide (Compound 192)

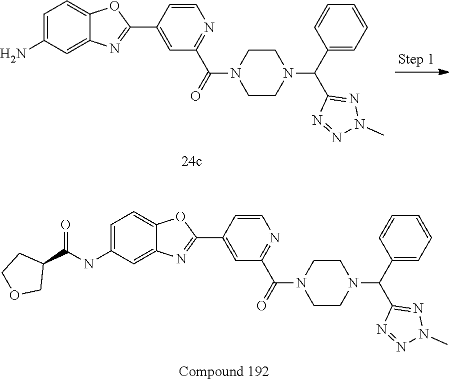 [SMILES: Cn1nnc(C(c2ccccc2)N2CCN(C(=O)c3cc(-c4nc5cc(N)ccc5o4)ccn3)CC2)n1 ; Cn1nnc(C(c2ccccc2)N2CCN(C(=O)c3cc(-c4nc5cc(NC(=O)[C@@H]6CCOC6)ccc5o4)ccn3)CC2)n1]

Step 1:

### (3R)—N-(2-(2-(4-((2-methyl-2H-tetrazol-5-yl)(phenyl)methyl)piperazine-1-carbonyl)pyridin-4-yl)benzo[d]oxazol-5-yl)tetrahydrofuran-3-carboxamide (Compound 192)

At room temperature, DMF (5 mL), (R)-tetrahydrofuran-3-carboxylic acid (42 mg, 0.36 mmol), HATU (138 mg, 0.36 mmol), and TEA (92 mg, 0.91 mmol) were successively added to compound 24C (150 mg, 0.30 mmol). The reaction was stirred for 1 hour. The reaction solution was poured into water (50 mL) and extracted with ethyl acetate (50 mL×2). The organic phase was concentrated, and the residue was separated and purified by HPLC to obtain compound 192 (36 mg, 20%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.73 min.

LC-MS (ESI): m/z=594.3 [M+H]⁺.

### Example 193

### (2R)—N-(2-(2-(4-((2-methyl-2H-tetrazol-5-yl)(phenyl)methyl)piperazine-1-carbonyl)pyridin-4-yl)benzo[d]oxazol-5-yl)tetrahydrofuran-2-carboxamide (Compound 193)

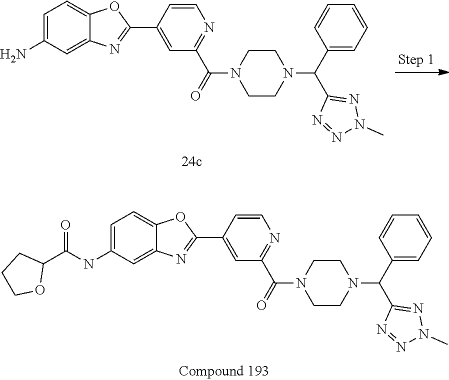 [SMILES: Cn1nnc(C(c2ccccc2)N2CCN(C(=O)c3cc(-c4nc5cc(N)ccc5o4)ccn3)CC2)n1 ; Cn1nnc(C(c2ccccc2)N2CCN(C(=O)c3cc(-c4nc5cc(NC(=O)C6CCCO6)ccc5o4)ccn3)CC2)n1]

Step 1:

### (2R)—N-(2-(2-(4-((2-methyl-2H-tetrazol-5-yl)(phenyl)methyl)piperazine-1-carbonyl)pyridin-4-yl)benzo[d]oxazol-5-yl)tetrahydrofuran-2-carboxamide (Compound 193)

At room temperature, DMF (5 mL), (R)-tetrahydrofuran-2-carboxylic acid (42 mg, 0.36 mmol), HATU (138 mg, 0.36 mmol), and TEA (92 mg, 0.91 mmol) were successively added to compound 24C (150 mg, 0.30 mmol). The reaction was stirred for 1 hour. The reaction solution was poured into water (50 mL) and extracted with ethyl acetate (50 mL×2). The organic phase was concentrated, and the residue was separated and purified by HPLC to obtain compound 193 (34 mg, 19%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 10.66 min.

LC-MS (ESI): m/z=594.3 [M+H]⁺.

¹H NMR (400 MHz, DMSO) δ 9.92 (s, 1H), 8.80 (d, 1H), 8.29 (s, 1H), 8.22-8.09 (m, 2H), 7.79 (s, 2H), 7.55-7.44 (m, 2H), 7.42-7.23 (m, 3H), 5.10 (s, 1H), 4.46-4.40 (m, 1H), 4.35 (s, 3H), 4.01 (dd, 1H), 3.86 (dd, 1H), 3.75-3.66 (m, 2H), 3.64 (s, 1H), 3.50-3.43 (m, 2H), 2.61-2.53 (m, 1H), 2.47-2.39 (m, 2H), 2.26-2.16 (m, 1H), 2.06-1.97 (m, 2H), 1.89-1.86 (m, 1H).

### Example 194

### 1-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide (Compound 194)

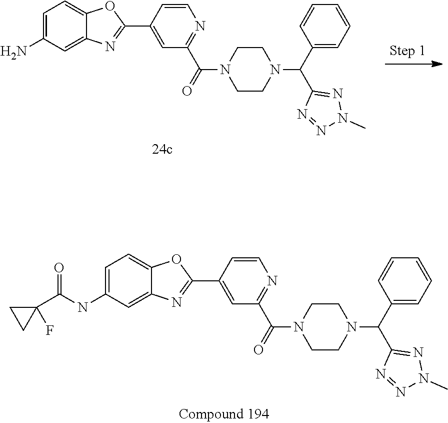 [SMILES: Cn1nnc(C(c2ccccc2)N2CCN(C(=O)c3cc(-c4nc5cc(N)ccc5o4)ccn3)CC2)n1 ; Cn1nnc(C(c2ccccc2)N2CCN(C(=O)c3cc(-c4nc5cc(NC(=O)C6(F)CC6)ccc5o4)ccn3)CC2)n1]

Step 1:

### 1-fluoro-N-(2-(2-(4-((2-methyl-2H-tetrazol-5-yl)(phenyl)methyl)piperazine-1-carbonyl)pyridin-4-yl)benzo[d]oxazol-5-yl)cyclopropane-1-carboxamide (Compound 194)

At room temperature, DMF (5 mL), 1-fluorocyclopropane-1-carboxylic acid (37.5 mg, 0.36 mmol), HATU (138 mg, 0.36 mmol) and TEA (92 mg, 0.91 mmol) were successively added to compound 24C (150 mg, 0.30 mmol). The reaction was stirred for 1 hour. The reaction solution was poured into water (50 mL) and extracted with ethyl acetate (50 mL×2). The organic phase was concentrated, and the residue was separated and purified by HPLC to obtain compound 194 (39 mg, 22%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.23 min.

LC-MS (ESI): m/z=582.3 [M+H]⁺.

¹H NMR (400 MHz, DMSO) δ 10.47 (s, 1H), 8.81 (d, 1H), 8.27 (s, 1H), 8.22-8.08 (m, 2H), 7.82 (s, 2H), 7.57-7.41 (m, 2H), 7.43-7.23 (m, 3H), 5.10 (s, 1H), 4.35 (s, 3H), 3.76-3.59 (m, 2H), 3.55-3.42 (m, 2H), 2.61-2.53 (m, 1H), 2.48-2.38 (m, 2H), 2.36-2.27 (m, 1H), 1.51-1.39 (m, 2H), 1.39-1.31 (m, 2H).

### Example 195

### 3,3-difluoro-N-(2-(2-(4-((2-methyl-2H-tetrazol-5-yl)(phenyl)methyl)piperazine-1-carbonyl)pyridin-4-yl)benzo[d]oxazol-5-yl)cyclobutane-1-carboxamide (Compound 195)

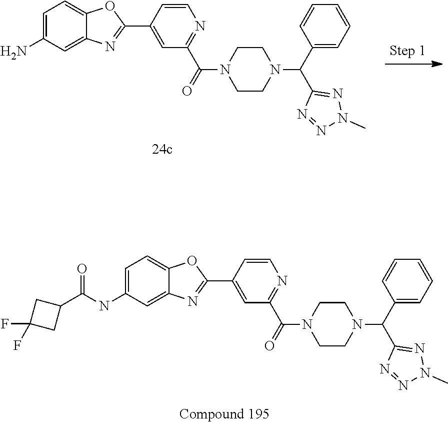 [SMILES: Cn1nnc(C(c2ccccc2)N2CCN(C(=O)c3cc(-c4nc5cc(N)ccc5o4)ccn3)CC2)n1 ; Cn1nnc(C(c2ccccc2)N2CCN(C(=O)c3cc(-c4nc5cc(NC(=O)C6CC(F)(F)C6)ccc5o4)ccn3)CC2)n1]

Step 1:

### 3,3-difluoro-N-(2-(2-(4-((2-methyl-2H-tetrazol-5-yl)(phenyl)methyl)piperazine-1-carbonyl)pyridin-4-yl)benzo[d]oxazol-5-yl)cyclobutane-1-carboxamide (Compound 195)

At room temperature, DMF (5 mL), 3,3-difluorocyclobutane-1-carboxylic acid (49 mg, 0.36 mmol), HATU (138 mg, 0.36 mmol) and TEA (92 mg, 0.91 mmol) were successively added to compound 24C (150 mg, 0.30 mmol). The reaction was stirred for 1 hour. The reaction solution was poured into water (50 mL) and extracted with ethyl acetate (50 mL×2). The organic phase was concentrated, and the residue was separated and purified by HPLC to obtain compound 195 (33 mg, 18%). Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.42 min.

LC-MS (ESI): m/z=614.2 [M+H]⁺.

¹H NMR (400 MHz, DMSO) δ 10.33 (s, 1H), 8.80 (d, J=5.2 Hz, 1H), 8.25 (s, 1H), 8.20-8.09 (m, 2H), 7.81 (d, J=8.9 Hz, 1H), 7.61 (d, J=9.0 Hz, 1H), 7.49 (d, J=7.2 Hz, 2H), 7.36 (t, J=7.4 Hz, 2H), 7.33-7.26 (m, 1H), 5.10 (s, 1H), 4.35 (s, 3H), 3.76-3.61 (m, 2H), 3.54-3.42 (m, 3H), 2.94-2.74 (m, 4H), 2.38-2.25 (m, 2H), 2.00 (dd, J=14.9, 6.7 Hz, 2H).

### Example 196

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclopropanecarboxamide

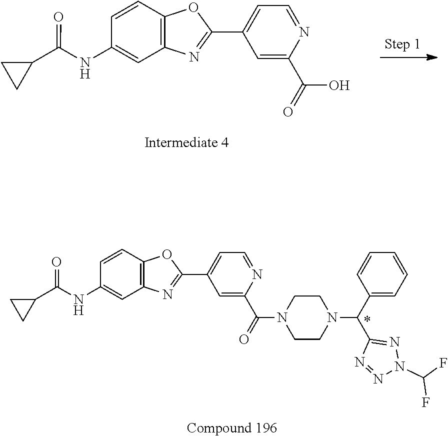 [SMILES: O=C(O)c1cc(-c2nc3cc(NC(=O)C4CC4)ccc3o2)ccn1 ; O=C(Nc1ccc2oc(-c3ccnc(C(=O)N4CCN(C(c5ccccc5)c5nnn(C(F)F)n5)CC4)c3)nc2c1)C1CC1]

Step 1:

### (R/S)—N-(2-(2-(4-((2-(difluoromethyl)-2H-tetrazol-5-yl)(phenyl)methyl)piperazine-1-carbonyl)pyridin-4-yl)benzo[d]oxazol-5-yl)cyclopropanecarboxamide (Compound 196)

Intermediate 4 (0.38 g, 1.17 mmol) and DMF (10 mL) were added, and DIPEA (0.45 g, 3.51 mmol), HATU (0.58 g, 1.52 mmol) and intermediate 7 (0.41 g, 1.40 mmol) were added. After the addition, the mixture was reacted at room temperature for 2 h. The reaction solution was adjusted to neutral by dropwise adding saturated aqueous ammonium chloride solution, and 30 ml of saturated aqueous sodium chloride solution was added. The mixture was extracted with ethyl acetate (25 ml×3). The organic layer was combined, dried, filtered and concentrated, and the residue was separated by column chromatography (dichloromethane/methanol (v/v)=50:1-10:1) to obtain compound 196 (0.10 g, 14%).

LC-MS (ESI): m/z=600.2 [M+H]⁺.

¹H NMR (400 MHz, CDCl₃) δ 8.71 (d, 1H), 8.34 (s, 1H), 8.07 (dd, 1H), 7.97 (s, 1H), 7.77 (s, 1H), 7.62-7.35 (m, 7H), 5.19 (s, 1H), 3.93 (m, 2H), 3.77 (m, 2H), 2.64 (m, 4H), 1.60-1.49 (m, 1H), 1.12 (dd, 2H), 0.87 (m, 2H).

### Example 197

### (1S,2S)-2-fluoro-N-(2-(2-(4-((R/S)-(5-(hydroxymethyl)-2H-tetrazol-2-yl)(phenyl)methyl)piperidine-1-carbonyl)pyridin-4-yl)benzo[d]oxazol-5-yl)cyclopropane-1-carboxamide

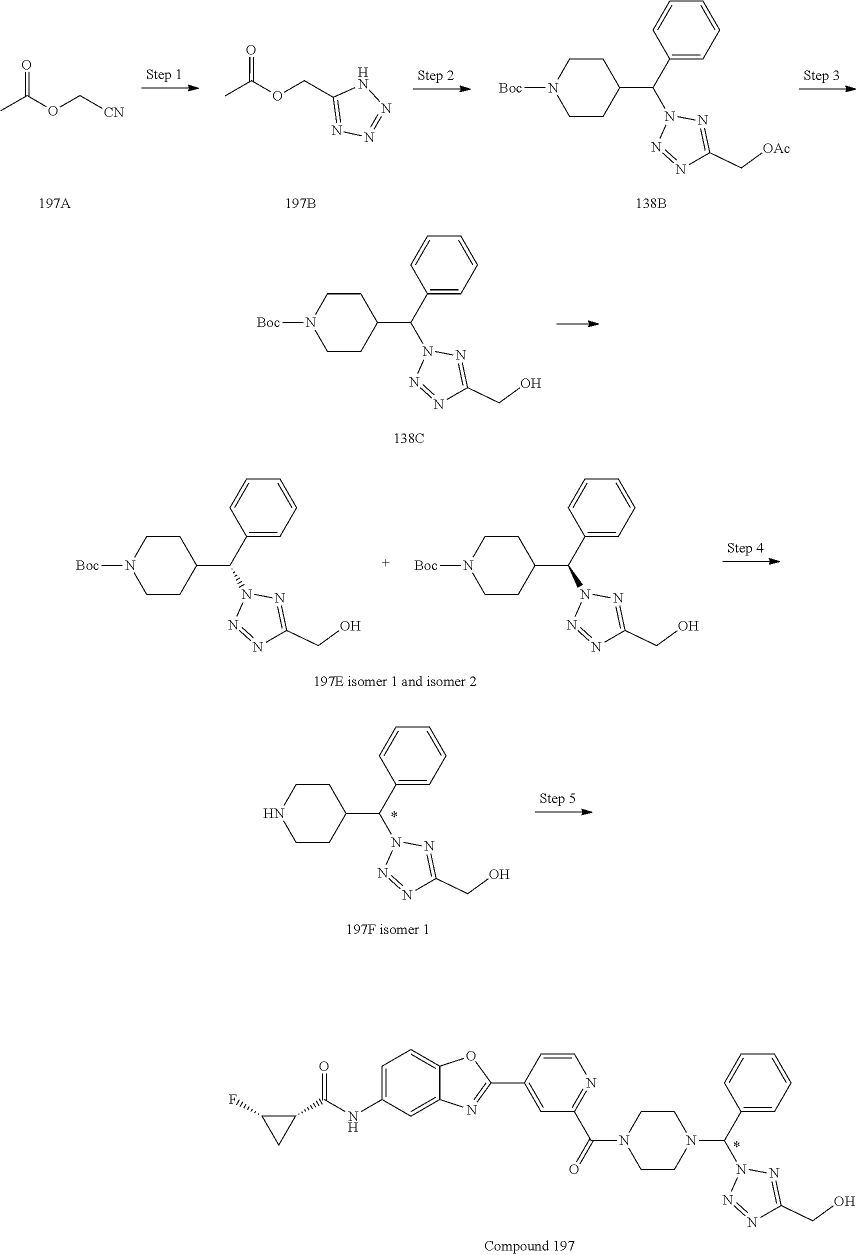

Step 1:

### (1H-tetrazol-5-yl)methyl Acetate (197B)

Under N₂ protection, methyl cyanoacetate (20 g, 0.2 mol) and TMSN₃ (35 g, 0.3 mol) were dissolved in chlorobenzene (200 ml), and a solution of TABF in tetrahydrofuran (100 ml, 0.1 mol) was slowly added. The reaction was warmed to 100° C. and stirred, and after 6 hours, the reaction solution was concentrated to remove most of chlorobenzene. The residue was diluted by adding ethyl acetate, washed with water and saturated brine, dried over anhydrous sodium sulfate and concentrated to obtain 197B as a viscous substance (25 g, 88%), which was directly used in the next reaction without purification.

LC-MS (ESI): m/z=143.2[M+H]⁺.

Step 2:

### tert-butyl 4-((5-(acetoxymethyl)-2H-tetrazol-2-yl)(phenyl)methyl)piperidine-1-carboxylate (138B)

Under N₂ protection, compound 197B (20 g, 0.14 mol), tert-butyl 4-(hydroxyl(phenyl)methyl)piperidine-1-carboxylate (29.1 g, 0.1 mol) and triphenylphosphine (39.3 g, 0.15 mol) were added to dichloromethane (300 ml), and DIAD (31.8 g, 0.15 mol) was added dropwise under ice-water bath cooling. After completion of the dropwise addition, the reaction was allowed to naturally warm to room temperature and stirred for two hours, and the reaction solution was diluted with dichloromethane, washed with water and saturated brine, dried over anhydrous sodium sulfate and concentrated to obtain a crude product of 138B, which was directly used in the next reaction.

LC-MS (ESI): m/z=416.2[M+H]⁺.

Step 3:

### tert-butyl (R)-4-((5-(hydroxymethyl)-2H-tetrazol-2-yl)(phenyl)methyl)piperidine-1-carboxylate

### tert-butyl (S)-4-((5-(hydroxymethyl)-2H-tetrazol-2-yl)(phenyl)methyl)piperidine-1-carboxylate (197E, Isomer 1 and Isomer 2)

The crude product of 138B (100 g) obtained from the previous step was dissolved in a mixed system of water (300 ml) and methanol (300 ml) at room temperature, and potassium carbonate (30 g, 0.22 mol) was added. The reaction was stirred at room temperature, and after two hours, the reaction solution was diluted with water, extracted with ethyl acetate, washed with water and saturated brine, dried over anhydrous sodium sulfate and concentrated to obtain the crude product, which was subjected to column chromatography (PE:EA=1:1) to obtain 138C (11 g, yield for two steps: 29.5%). 1.5 g of the compound was subjected to chiral resolution to obtain 197E isomer 1 (600 mg) and 197E isomer 2 (500 mg).

Resolution Conditions:

preparation instrument: Gilson GX-281; chiral column: CHIRALPAK AD-H, 5 m, 20 mm×250 mm;
mobile phase system: n-hexane:isopropanol, isocratic elution using 10% isopropanol; retention time: 25.2 min for isomer 1 and 28.1 min for isomer 2;

LC-MS (ESI): m/z=374.2 [M+H]⁺.

Step 4:

### (R/S)-(2-(phenyl(piperidin-4-yl)methyl)-2H-tetrazol-5-yl)methanol (197F, Isomer 1)

Compound 197E isomer 1 (400 mg, 1.07 mmol) was added to ethyl acetate (4 ml), and 6 mol/L HCl/dioxane (4 ml) was added dropwise. The mixture was stirred at room temperature for 4 hours and then concentrated to dryness to obtain the hydrochloride of compound 197F isomer 1, which was directly used in the next reaction.

LC-MS (ESI): m/z=274.2 [M+H]⁺.

Step 5:

### (1S,2S)-2-fluoro-N-(2-(2-(4-((R/S)-(5-(hydroxymethyl)-2H-tetrazol-2-yl)(phenyl)methyl)piperidine-1-carbonyl)pyridin-4-yl)benzo[d]oxazol-5-yl)cyclopropane-1-carboxamide (Compound 197)

Compound 197F isomer 1 (330 mg, 1.07 mmol) and 117F (375 mg, 1.1 mmol) obtained from the previous step were added to dichloromethane (5 ml); DIEA (230 mg, 2.0 mmol) was added dropwise; and HATU (570 mg, 1.5 mmol) was added in portions. The mixture was stirred at room temperature for 2 hours, and the reaction was poured into water. The resulting solution was extracted with dichloromethane, washed with saturated brine, dried over anhydrous sodium sulfate and concentrated, and the residue was subjected to column chromatography (PE:EA=1:1) to obtain target compound 197 (180 mg, yield for two steps: 28.2%).

LC-MS (ESI): m/z=597.2 [M+H]⁺.

¹H NMR (400 MHz, DMSO) δ 10.47 (s, 1H), 8.81-8.79 (m, 1H), 8.24 (s, 1H), 8.16 (s, 1H), 8.14-8.12 (m, 1H), 7.80 (s, 1H), 7.64-7.61 (m, 2H), 7.59-7.57 (m, 1H), 7.46-7.33 (m, 3H), 6.00-5.98 (m, 1H), 5.6-5.52 (m, 1H), 5.06-4.86 (m, 1H), 4.70-4.63 (m, 2H), 4.51-4.46 (m, 1H), 3.75-3.71 (m, 1H), 3.12-3.05 (m, 1H), 2.92-2.82 (m, 2H), 2.08-2.01 (m, 1H), 1.72-1.62 (m, 1H), 1.38-1.02 (m, 5H).

### Example 198

### (1S,2S)-2-fluoro-N-(2-(2-(4-((S/R)-(5-(hydroxymethyl)-2H-tetrazol-2-yl)(phenyl)methyl)piperidine-1-carbonyl)pyridin-4-yl)benzo[d]oxazol-5-yl)cyclopropane-1-carboxamide

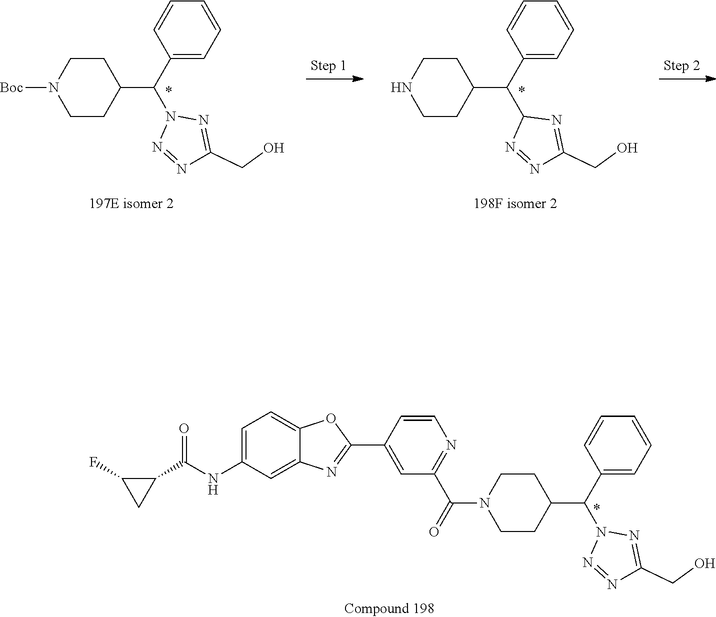 [SMILES: CC(C)(C)OC(=O)N1CCC(C(c2ccccc2)n2nnc(CO)n2)CC1 ; OCC1=NC(C(c2ccccc2)C2CCNCC2)N=N1 ; O=C(Nc1ccc2oc(-c3ccnc(C(=O)N4CCC(C(c5ccccc5)n5nnc(CO)n5)CC4)c3)nc2c1)[C@@H]1C[C@@H]1F]

197E isomer 2 and 117F were used as raw materials to obtain compound 198 (85 mg, yield for two steps: 26.6%) with reference to the synthetic method of example 197.

LC-MS (ESI): m/z=597.2 [M+H]⁺.

¹H NMR (400 MHz, DMSO) δ 10.47 (s, 1H), 8.81-8.79 (m, 1H), 8.24 (s, 1H), 8.16 (s, 1H), 8.14-8.12 (m, 1H), 7.80 (s, 1H), 7.64-7.61 (m, 2H), 7.59-7.57 (m, 1H), 7.46-7.33 (m, 3H), 6.00-5.98 (m, 1H), 5.6-5.52 (m, 1H), 5.06-4.86 (m, 1H), 4.70-4.63 (m, 2H), 4.51-4.46 (m, 1H), 3.75-3.71 (m, 1H), 3.12-3.05 (m, 1H), 2.92-2.82 (m, 2H), 2.08-2.01 (m, 1H), 1.72-1.62 (m, 1H), 1.38-1.02 (m, 5H).

### Example 199

### (2-((R/S)-(1-(4-(5-((1S,2S)-2-fluorocyclopropane-1-carboxamido)benzo[d]oxazol-2-yl)picolinoyl)piperidin-4-yl)(phenyl)methyl)-2H-tetrazol-5-yl)methyl Acetate

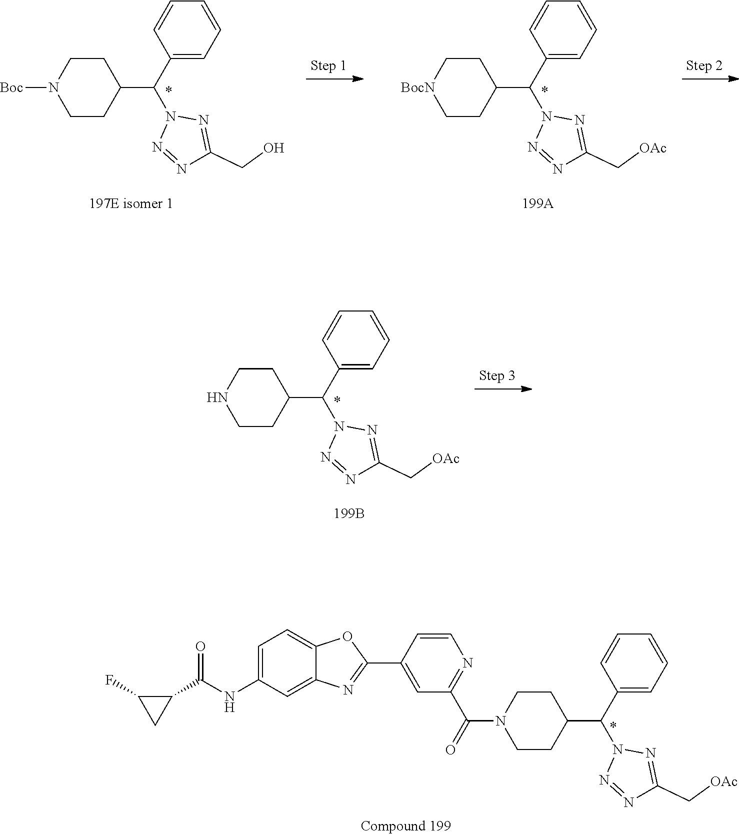 [SMILES: CC(C)(C)OC(=O)N1CCC(C(c2ccccc2)n2nnc(CO)n2)CC1 ; CC(=O)OCc1nnn(C(c2ccccc2)C2CCN(C(=O)OC(C)(C)C)CC2)n1 ; CC(=O)OCc1nnn(C(c2ccccc2)C2CCNCC2)n1 ; CC(=O)OCc1nnn(C(c2ccccc2)C2CCN(C(=O)c3cc(-c4nc5cc(NC(=O)[C@@H]6C[C@@H]6F)ccc5o4)ccn3)CC2)n1]

Step 1:

### tert-butyl (R/S)-4-((5-(acetoxymethyl)-2H-tetrazol-2-yl)(phenyl)methyl)piperidine-1-carboxylate (199A)

Under nitrogen protection, dichloromethane (6 mL), acetic anhydride (273 mg, 2.68 mmol) and triethylamine (0.56 ml, 4.02 mol) were successively added to intermediate 197E isomer 1 (500 mg, 1.34 mmol), and the reaction was stirred at room temperature for 1 hour. Aqueous solution (20 mL) was added, and the resulting solution was extracted ethyl acetate (20 mL×2). The organic phase was concentrated to obtain 199A (500 mg, 90%).

LC-MS (ESI): m/z=416.2 [M+H]⁺.

Step 2:

### (R/S)-(2-(phenyl(piperidin-4-yl)methyl)-2H-tetrazol-5-yl)methyl Acetate (199B)

At room temperature, 2 M hydrochloric acid (10 mL) was added to compound 199A (500 mg, 1.2 mmol). The reaction was stirred at room temperature for 1 hour. The reaction solution was concentrated to obtain 199B (240 mg, 63%).

LC-MS (ESI): m/z=316.2 [M+H]⁺.

Step 3:

### (2-((R/S)-(1-(4-(5-((1S,2S)-2-fluorocyclopropane-1-carboxamido)benzo[d]oxazol-2-yl)picolinoyl)piperidin-4-yl)(phenyl)methyl)-2H-tetrazol-5-yl)methyl acetate (Compound 199)

At room temperature, DMF (5 mL), 117F (390 mg, 1.14 mmol), HATU (330 mg, 1.14 mmol) and DIPEA (0.2 ml, 2.3 mmol) were successively added to compound 199B (240 mg, 0.76 mmol). The reaction was stirred for 2 hours. The reaction solution was poured into water (15 mL) and extracted with ethyl acetate (20 mL×2). The organic phase was concentrated, and the residue was separated and purified by silica gel column chromatography (eluent: PE:EA=65%-70%) to obtain compound 199 (100 mg, 21%).

¹H NMR (400 MHz, MeOD) δ 8.79 (s, 1H), 8.29 (s, 1H), 8.20 (s, 2H), 7.71-7.55 (m, 4H), 7.46-7.32 (m, 3H), 5.84 (d, 1H), 5.33 (d, 2H), 4.95-4.90 (m, 1H), 4.71-4.63 (m, 1H), 3.88-3.82 (m, 1H), 3.16-3.11 (m, 1H), 3.02-2.91 (m, 2H), 2.13-1.99 (m, 3H), 1.83-1.74 (m, 1H), 1.45-1.16 (m, 6H).

LC-MS (ESI): m/z=639.3 [M+H]⁺.

### Example 200

### (1S,2S)-2-fluoro-N-(2-(2-(4-((R/S)-(5-(methoxymethyl)-2H-tetrazol-2-yl)(phenyl)methyl)piperidine-1-carbonyl)pyridin-4-yl)benzo[d]oxazol-5-yl)cyclopropane-1-carboxamide

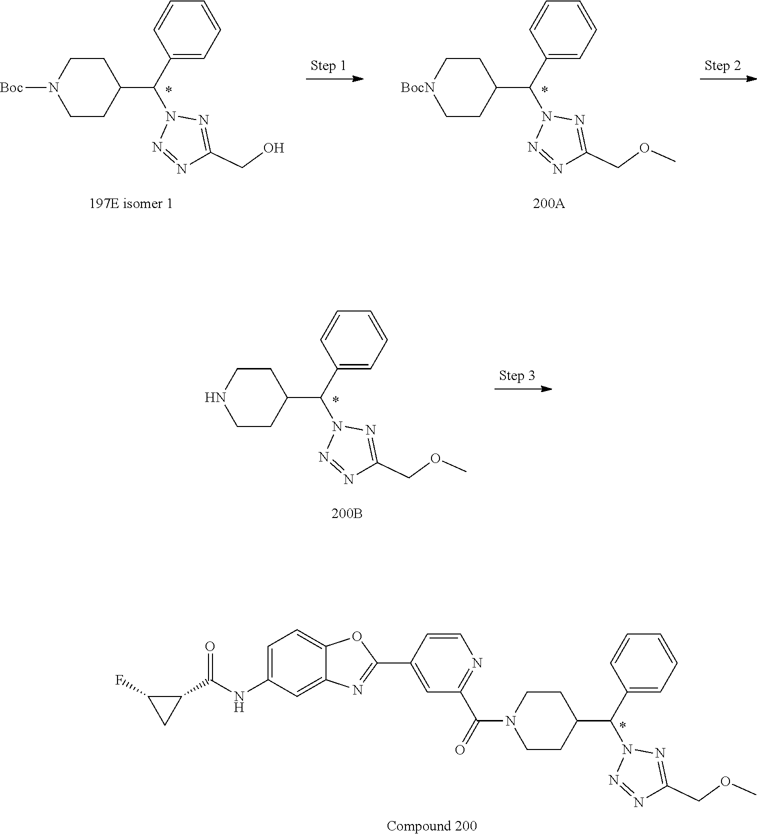 [SMILES: CC(C)(C)OC(=O)N1CCC(C(c2ccccc2)n2nnc(CO)n2)CC1 ; COCc1nnn(C(c2ccccc2)C2CCN(C(=O)OC(C)(C)C)CC2)n1 ; COCc1nnn(C(c2ccccc2)C2CCNCC2)n1 ; COCc1nnn(C(c2ccccc2)C2CCN(C(=O)c3cc(-c4nc5cc(NC(=O)[C@@H]6C[C@@H]6F)ccc5o4)ccn3)CC2)n1]

Step 1:

### tert-butyl (R/S)-4-((5-(methoxymethyl)-2H-tetrazol-2-yl)(phenyl)methyl)piperidine-1-carboxylate (200A)

Under nitrogen protection, dichloromethane (5 mL), methanol (5 mL) and TMSCHN₂ (trimethylsilazodimethane) (3.06 g, 26.8 mmol) were successively added to 197E isomer 1 (1 g, 2.68 mmol). The reaction was stirred at room temperature for 3 hours. 2 drops of acetic acid solution were added. The reaction solution was concentrated, and the residue was separated and purified by silica gel column chromatography (eluent: PE:EA=15%-20%) to obtain 200A (420 mg, 40.5%).

LC-MS (ESI): m/z=388.2 [M+H]⁺.

Step 2:

### (R/S)-4-((5-(methoxymethyl)-2H-tetrazol-2-yl)(phenyl)methyl)piperidine (200B)

At room temperature, 2 M hydrochloric acid (10 mL) was added to compound 200A (420 mg, 1.08 mmol). The reaction was stirred at room temperature for 1 hour. The reaction solution was concentrated to obtain 200B (320 mg, 92%).

LC-MS (ESI): m/z=288.2 [M+H]⁺.

Step 3:

### (1S,2S)-2-fluoro-N-(2-(2-(4-((R/S)-(5-(methoxymethyl)-2H-tetrazol-2-yl)(phenyl)methyl)piperidine-1-carbonyl)pyridin-4-yl)benzo[d]oxazol-5-yl)cyclopropane-1-carboxamide (Compound 200)

At room temperature, DMF (5 mL), 117F (510 mg, 1.5 mmol), HATU (640 mg, 1.5 mmol) and DIPEA (0.62 ml, 3 mmol) were successively added to compound 200B (320 mg, 1.01 mmol). The reaction was stirred for 2 hours. The reaction solution was poured into water (15 mL) and extracted with ethyl acetate (20 mL×2). The organic phase was concentrated, and the residue was separated and purified by silica gel column chromatography (eluent: PE: EA=95%-100%) to obtain compound 200 (320 mg, 50%).

¹H NMR (400 MHz, MeOD) δ 8.79 (m, 1H), 8.28 (s, 1H), 8.17 (s, 2H), 7.70-7.55 (m, 4H), 7.47-7.34 (m, 3H), 5.86 (d, 1H), 5.03-4.90 (m, 1H), 4.79-4.75 (m, 1H), 4.73-4.68 (m, 1H), 3.98-3.84 (m, 1H), 3.53-3.46 (m, 3H), 3.25-2.91 (m, 4H), 2.13-2.03 (m, 1H), 1.87-1.77 (m, 1H), 1.65-1.43 (m, 4H), 1.39-1.28 (m, 2H).

LC-MS (ESI): m/z=611.3 [M+H]⁺.

### Example 201

### (1S,2S)-2-fluoro-N-(2-(2-(4-((R/S)-(5-(methyl-d3)-2H-tetrazol-2-yl)(phenyl)methyl)piperidine-1-carbonyl)pyridin-4-yl)benzo[d]oxazol-5-yl)cyclopropane-1-carboxamide

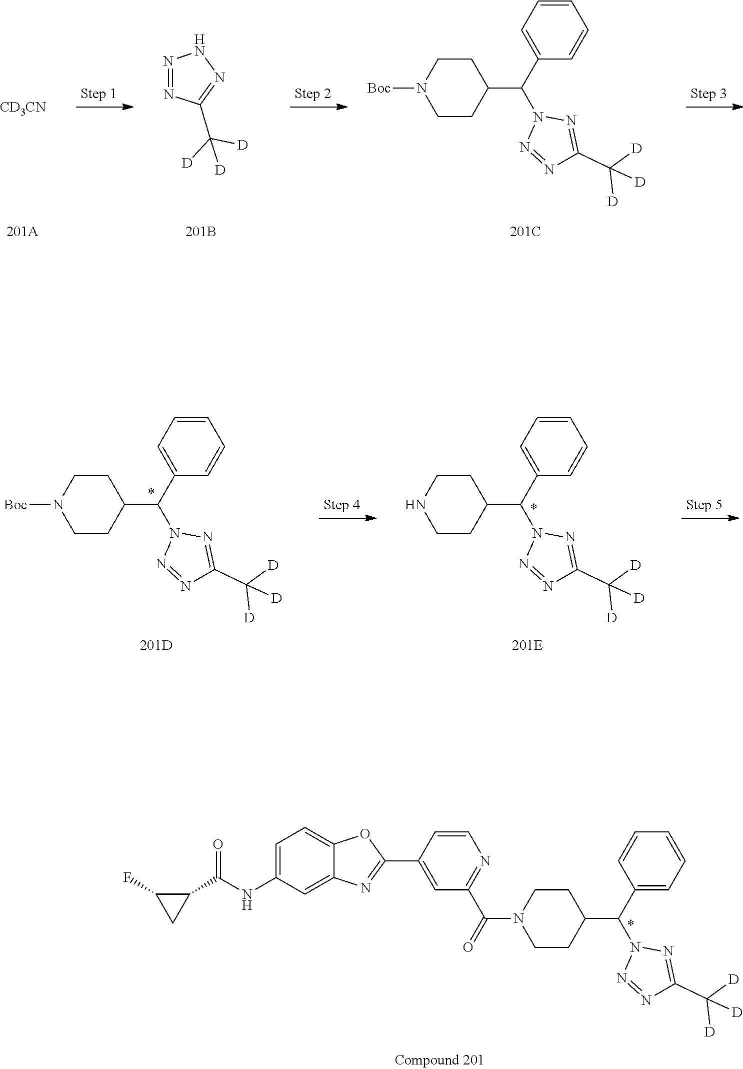 [SMILES: "CD3CN" ; [2H]C([2H])([2H])c1nn[nH]n1 ; [2H]C([2H])([2H])c1nnn(C(c2ccccc2)C2CCN(C(=O)OC(C)(C)C)CC2)n1 ; [2H]C([2H])([2H])c1nnn(C(c2ccccc2)C2CCN(C(=O)OC(C)(C)C)CC2)n1 ; [2H]C([2H])([2H])c1nnn(C(c2ccccc2)C2CCNCC2)n1 ; [2H]C([2H])([2H])c1nnn(C(c2ccccc2)C2CCN(C(=O)c3cc(-c4nc5cc(NC(=O)[C@@H]6C[C@@H]6F)ccc5o4)ccn3)CC2)n1]

Step 1:

### 5-(methyl-d3)-2H-tetrazole (201B)

Under nitrogen protection, compound deuterated acetonitrile (5 g, 0.11 mol), toluene (5 mL), sodium azide (8.86 g, 0.13 mol) and triethylamine hydrochloride (23.4 g, 0.17 mmol) were successively added to a single-necked flask, and the reaction was stirred at 100° C. overnight. The reaction was cooled to room temperature and quenched by adding dilute hydrochloric acid aqueous solution. The aqueous phase was washed with dichloromethane (50 mL×2), soaked with sodium hypochlorite aqueous solution overnight and then poured into a waste liquid tank. The organic phase was combined, dried over anhydrous sodium sulfate and concentrated under reduced pressure to obtain compound 201B (1.7 g, 10.12%) as a light yellow solid from the residue.

¹H NMR (400 MHz, DMSO-d₆) δ 15.93 (s, 1H).

Step 2:

### tert-butyl-4-((5-(methyl-d3)-2H-tetrazol-2-yl)(phenyl)methyl)piperidine-1-carboxylate (201C)

Under nitrogen protection, compound 201B (2.1 g, 24 mmol), dichloromethane (20 mL), tert-butyl 4-(hydroxyl(phenyl)methyl)piperidine-1-carbonate 85A (7.03 g, 24 mmol) and triphenylphosphine (7.54 g, 28.8 mmol) were successively added to a single-necked flask. DIAD (5.82 g, 28.8 mmol) was added dropwise with stirring under an ice bath. After the dropwise addition was completed, the mixture was warmed to room temperature, reacted for another 3 h and concentrated under reduced pressure to obtain a crude product from the residue, and the crude product was separated by column chromatography (eluent: EA/PE=1/4) to obtain compound 201C as a light yellow solid (3.5 g, 40.3%).

LC-MS (ESI): m/z=174.3[M−186]⁺.

Step 3:

### tert-butyl-(R/S)-4-((5-(methyl-d3)-2H-tetrazol-2-yl)(phenyl)methyl)piperidine-1-carboxylate (201D)

Compound 201C was subjected to SFC resolution to obtain compound 201D as an oil (about 1.86 g).

SFC resolution conditions were as follows:

instrument: MG II preparative SFC (SFC-14); column type: ChiralCel OJ, 250×30 mm I.D., 5 μm; the samples were dissolved in about 40 ml of methanol/DCM; mobile phase: A for CO₂ and B for ethanol; gradient: mobile phase B: 15%; flow rate: 60 mL/min; column pressure: 100 bar; column temperature: 38° C.; wavelength: 220 nm; elution time: 25 min; retention time for compound 201D: 1.791 min; sample injection: 1.1 mL each time.

Step 4:

### (R/S)-4-((5-(methyl-d3)-2H-tetrazol-2-yl)(phenyl)methyl)piperidine (201E)

Compound 201D (1.86 g, 2.16 mmol), dichloromethane (20 mL) and trifluoroacetic acid (6 mL) were successively added to a single-necked flask. The reaction was stirred at room temperature for 2 h and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure to obtain compound 201E (1.2 g, 89.3%).

LC-MS (ESI): m/z=261.3[M+H]⁺.

Step 5:

### (1S,2S)-2-fluoro-N-(2-(2-(4-((R/S)-(5-(methyl-d3)-2H-tetrazol-2-yl)(phenyl)methyl)piperidine-1-carbonyl)pyridin-4-yl)benzo[d]oxazol-5-yl)cyclopropane-1-carboxamide (Compound 201)

Under nitrogen protection, compound 117F (0.65 g, 1.9 mmol), DMF (10 mL), TCFH (tetramethylchlorourea hexafluorophosphate) (0.64 g, 2.3 mmol), methyl imidazole (0.5 g, 6 mmol) and compound 201E (500 mg, 1.9 mmol) were successively added to a single-necked flask, and the mixture was stirred at room temperature for 0.5 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by high-performance liquid chromatography to obtain compound 201 as a light yellow solid (0.5 g, 45%).

Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 12.81 min.

¹H NMR (400 MHz, CDCl₃) δ 8.73 (s, 1H), 8.33 (s, 1H), 8.15-8.13 (m, 1H), 8.02 (s, 1H), 7.81 (s, 1H), 7.52-7.50 (m, 4H), 7.38-7.33 (m, 2H), 5.55-5.52 (m, 1H), 4.93-4.74 (m, 2H), 3.86-3.82 (m, 1H), 3.22-3.13 (m, 1H), 2.90-2.84 (m, 2H), 2.42-2.23 (m, 2H), 1.86-1.85 (m, 1H), 1.47-1.39 (m, 3H), 1.28-1.24 (m, 2H).

LC-MS (ESI): m/z=584.3 [M+H]⁺.

### Example 202

### (1S,2S)-2-fluoro-N-(2-(2-(4-((R/S)-(4-fluorophenyl)(5-methyl-2H-tetrazol-2-yl)methyl)piperidine-1-carbonyl)pyridin-4-yl)benzo[d]oxazol-5-yl)cyclopropane-1-carboxamide

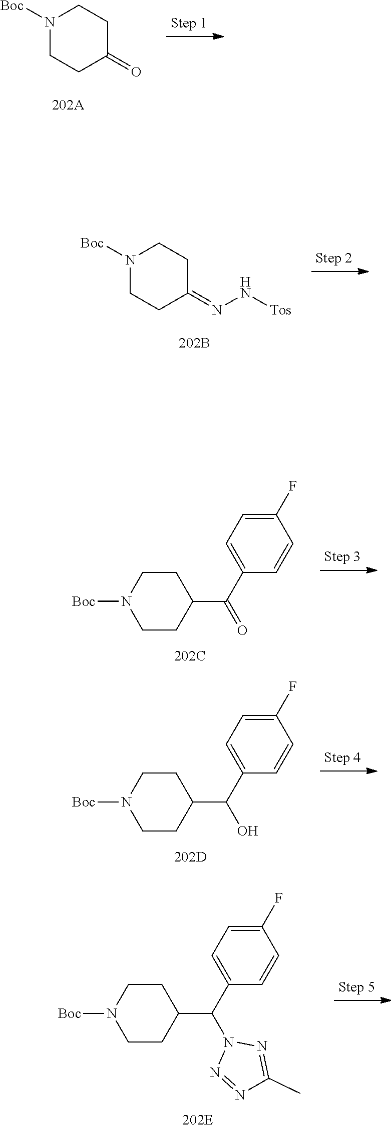

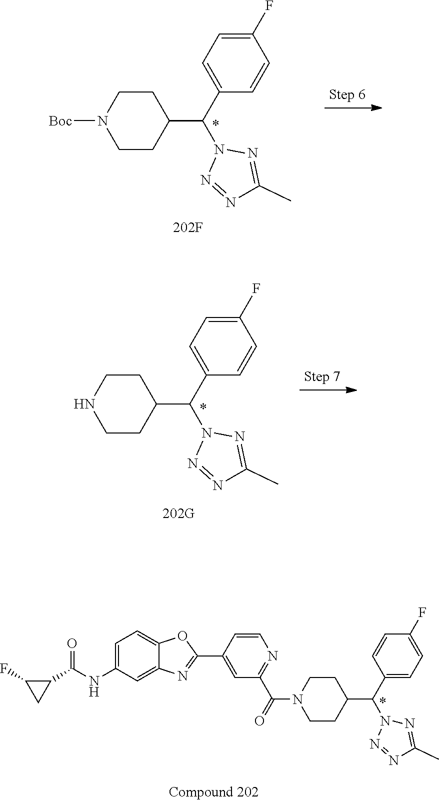

Step 1:

### tert-butyl 4-(2-tosylhydrazineylidene)piperidine-1-carboxylate (202B)

Compound methanol (30 mL), N-tert-butoxycarbonyl-4-piperidone (10 g, 50.2 mmol) and p-toluenesulfonyl hydrazide (9.35 g, 50 mmol) were successively added to a single-necked flask, and the reaction was stirred at room temperature overnight. After TLC showed that the reaction was completed, the reaction solution was concentrated under reduced pressure to obtain compound 202B as a white solid (17.5 g, 95%) from the residue. The compound can be directly used in the next reaction without purification. 1241 LC-MS (ESI): m/z=368.3[M+1]*.

Step 2:

### tert-butyl 4-(4-fluorobenzoyl)piperidine-1-carboxylate (202C)

Under nitrogen protection, compound 202B (6 g, 16.33 mmol), 1,4-dioxane (25 mL), cesium carbonate (6.5 g, 20 mmol) and p-fluorobenzaldehyde (2.43 g, 19.6 mmol) were successively added to a single-necked flask, and the reaction was stirred at 100° C. for 17 h. After TLC showed that the reaction was completed, the reaction was stopped, cooled to room temperature, filtered and concentrated under reduced pressure to obtain a crude product from the residue, and the crude product was separated by column chromatography (eluent: EA/PE=1/6) to obtain compound 202C as a light yellow solid (3.5 g, 69.74%).

LC-MS (ESI): m/z=308.1[M+1]⁺.

Step 3:

### tert-butyl 4-((4-fluorophenyl)(hydroxy)methyl)piperidine-1-carboxylate (202D)

Compound 202C (3.5 g, 11.4 mmol), methanol (25 mL) and sodium borohydride (0.43 g, 11.4 mmol) were successively added to a 50 ml single-necked flask at 0° C., and the reaction was stirred for 1 h. After TLC showed that the reaction was completed, the reaction was stopped. Water (10 mL) was added, and the resulting solution was concentrated under reduced pressure to obtain an aqueous phase, which was extracted with ethyl acetate. The organic phase was concentrated under reduced pressure to obtain compound 202D as a white solid (3.45 g, 98%).

LC-MS (ESI): m/z=192.3[M−117]⁺.

Step 4:

### tert-butyl 4-((4-fluorophenyl)(5-methyl-2H-tetrazol-2-yl)methyl)piperidine-1-carboxylate (202E)

Under nitrogen protection, compound 202D (3.45 g, 11.15 mmol), dichloromethane (25 mL), methyl tetrazole (1.13 g, 13.38 mmol) and triphenylphosphine (3.5 g, 13.38 mmol) were successively added to a single-necked flask, and DIAD (2.7 g, 13.38 mmol) was added dropwise with stirring under an ice bath. After the dropwise addition was completed, the mixture was warmed to room temperature, reacted for another 3 h and concentrated under reduced pressure to obtain a crude product from the residue, and the crude product was separated by column chromatography (eluent: EA/PE=1/4) to obtain compound 202E as a light yellow solid (1.98 g, 47.3%).

LC-MS (ESI): m/z=192.3[M−183]⁺.

Step 5:

### tert-butyl-(R)-4-((4-fluorophenyl)(5-methyl-2H-tetrazol-2-yl)methyl)piperidine-1-carboxylate (202F)

Compound 202E was subjected to SFC resolution to obtain compound 202F (a compound (about 0.85 g) as an oil).

SFC resolution conditions were as follows:

instrument: MG II preparative SFC (SFC-14); column type: ChiralCel OJ, 250×30 mm I.D., 5 μm; the samples were dissolved in about 40 ml of methanol/DCM; mobile phase: A for CO₂ and B for ethanol; gradient: mobile phase B: 15%; flow rate: 60 mL/min; column pressure: 100 bar; column temperature: 38° C.; wavelength: 220 nm; elution time: 25 min; retention time for compound 202F: 1.918 min; sample injection: 1.1 mL each time.

Step 6:

### (R/S)-4-((4-fluorophenyl)(5-methyl-2H-tetrazol-2-yl)methyl)piperidine (202G)

Compound 202F (0.85 g, 2.26 mmol), dichloromethane (20 mL) and trifluoroacetic acid (6 mL) were successively added to a single-necked flask. The reaction was stirred at room temperature for 2 h and adjusted to pH=8-9 by dropwise adding saturated aqueous sodium carbonate solution. The residue was extracted with dichloromethane (50 mL×2). The combined organic phase was dried over anhydrous sodium sulfate and concentrated under reduced pressure to obtain compound 202G (0.6 g, 96.26%).

LC-MS (ESI): m/z=276.3[M+H]⁺.

Step 7:

### (1S,2S)-2-fluoro-N-(2-(2-(4-((R/S)-(4-fluorophenyl)(5-methyl-2H-tetrazol-2-yl)methyl)piperidine-1-carbonyl)pyridin-4-yl)benzo[d]oxazol-5-yl)cyclopropane-1-carboxamide (Compound 202)

Under nitrogen protection, compound 117F (0.35 g, 1 mmol), DMF (10 mL), TCFH (0.42 g, 1.5 mmol), methyl imidazole (0.25 g, 3 mmol) and compound 202G (0.28 g, 1 mmol) were successively added to a single-necked flask, and the mixture was stirred at room temperature for 0.5 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by preparative high-performance liquid chromatography to obtain compound 202 as a light yellow solid (0.25 g, 40%).

Preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time: 13.02 min.

¹H NMR (400 MHz, CDCl₃) δ 8.73-8.72 (m, 1H), 8.29 (s, 1H), 8.09-8.07 (m, 1H), 8.02-7.99 (m, 1H), 7.90-7.87 (m, 1H), 7.55-7.50 (m, 4H), 7.11-7.02 (m, 2H), 5.53-5.50 (m, 1H), 4.91-4.73 (m, 2H), 3.94-3.91 (m, 1H), 3.15-3.09 (m, 1H), 2.88 (s, 3H), 2.55-2.50 (m, 2H), 2.00-1.81 (m, 1H), 1.59-1.40 (m, 4H), 1.39-1.20 (m, 2H).

LC-MS (ESI): m/z=599.2 [M+H]⁺.

### Examples 203 and 204

### (1S,2S)-2-fluoro-N-(2-(2-(4-((R)-(5-methyl-2H-tetrazol-2-yl)(thiophen-3-yl)methyl)piperidine-1-carbonyl)pyridin-4-yl)benzo[d]oxazol-5-yl)cyclopropanecarboxamide and

### (1S,2S)-2-fluoro-N-(2-(2-(4-((S)-(5-methyl-2H-tetrazol-2-yl)(thiophen-3-yl)methyl)piperidine-1-carbonyl)pyridin-4-yl)benzo[d]oxazol-5-yl)cyclopropanecarboxamide

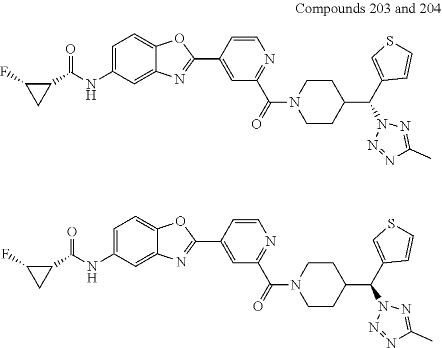

Compound 202A and 3-thiophene carbaldehyde were used as raw materials to obtain compounds 203 and 204 according to the synthetic method of compound 202.

Compound 203: 1H NMR (400 MHz, CDCl3) δ 8.72-8.71 (m, 1H), 8.22-8.14 (m, 2H), 8.05-8.03 (m, 1H), 7.94 (s, 1H), 7.51-7.26 (m, 5H), 5.72-5.69 (m, 1H), 4.90-4.70 (m, 2H), 3.95-3.89 (m, 1H), 3.12-3.05 (m, 1H), 2.86-2.72 (m, 2H), 2.56-2.50 (m, 3H), 1.96-1.84 (m, 2H), 1.51-1.13 (m, 5H).

LC-MS (ESI): m/z=587.2 [M+H]⁺.

Compound 204: 1H NMR (400 MHz, CDCl3) δ 8.72-8.71 (m, 1H), 8.22-8.18 (m, 2H), 8.04-8.03 (m, 1H), 7.94 (s, 1H), 7.51-7.26 (m, 5H), 5.72-5.69 (m, 1H), 4.89-4.69 (m, 2H), 3.93-3.89 (m, 1H), 3.12-3.06 (m, 1H), 2.86-2.77 (m, 2H), 2.56-2.50 (m, 3H), 1.96-1.89 (m, 1H), 1.51-1.13 (m, 6H).

LC-MS (ESI): m/z=587.2 [M+H]⁺.

### Example 205

### (1S,2S)-2-fluoro-N-[2-[2-[4-[(R)-(5-methyltetrazol-2-yl)-(2,3,4,5,6-pentadeuteriophenyl)methyl]piperidine-1-carbonyl]-4-pyridyl]-1,3-benzoxazol-5-yl]cyclopropanecarboxamide

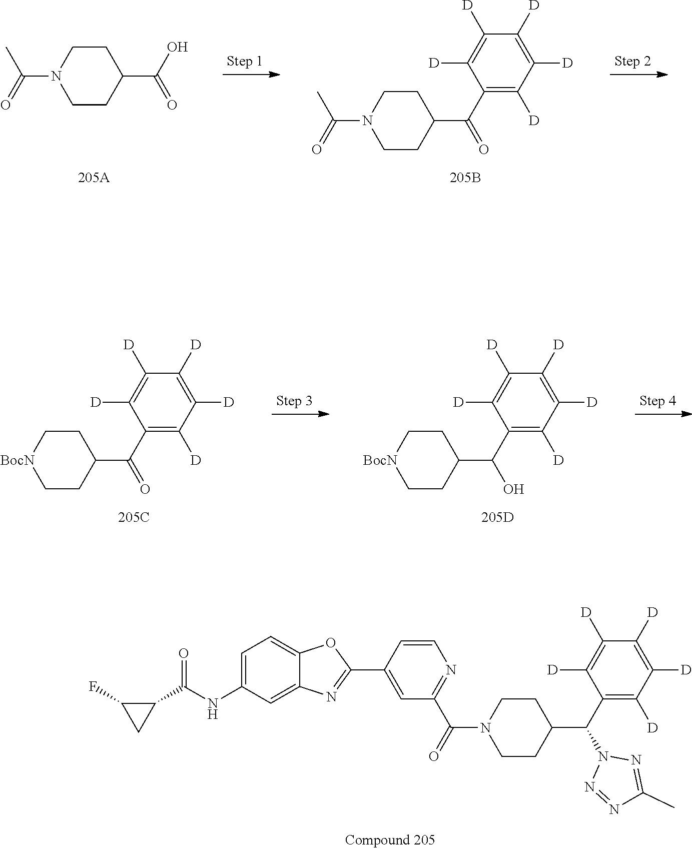 [SMILES: CC(=O)N1CCC(C(=O)O)CC1 ; [2H]c1c([2H])c([2H])c(C(=O)C2CCN(C(C)=O)CC2)c([2H])c1[2H] ; [2H]c1c([2H])c([2H])c(C(=O)C2CCN(C(=O)OC(C)(C)C)CC2)c([2H])c1[2H] ; [2H]c1c([2H])c([2H])c(C(O)C2CCN(C(=O)OC(C)(C)C)CC2)c([2H])c1[2H] ; [2H]c1c([2H])c([2H])c([C@@H](C2CCN(C(=O)c3cc(-c4nc5cc(NC(=O)[C@@H]6C[C@@H]6F)ccc5o4)ccn3)CC2)n2nnc(C)n2)c([2H])c1[2H]]

Step 1:

### 1-[4-(2,3,4,5,6-pentadeuteriobenzoyl)-1-piperidyl]ethanone (205B)

1-acetyl-4-piperidine carboxylic acid (205A)(17.1 g, 0.1 mol) was dissolved in 1,2-dichloroethane (40 mL), and a solution of thionyl chloride (13.1 g, 0.11 mol) in 1,2-dichloroethane (20 mL) was added dropwise at 40° C. After the dropwise addition was completed, the mixture was warmed to 65° C., reacted for another 1 h and cooled to room temperature. Then benzene-d6 (20 mL) was added and mixed evenly. Aluminum trichloride (26.7 g, 0.2 mol) was added in portions to the system. After the addition was completed, the reaction was reacted at room temperature for another 6 h. After the reaction was completed, the system was added to ice water, and the mixture was extracted with dichloromethane, washed with saturated brine, dried over anhydrous sodium sulfate and concentrated under reduced pressure; and then the residue was separated by silica gel column chromatography (EA/PE=10%-50%) to obtain compound 205B (18.2 g, 77%).

LC-MS (ESI): m/z=237.2[M+H]⁺.

Step 2:

### tert-butyl 4-(2,3,4,5,6-pentadeuteriobenzoyl)piperidine-1-carboxylate (205C)

1-[4-(2,3,4,5,6-pentadeuteriobenzoyl)-1-piperidyl]ethanone (205B) (18.2 g, 77 mol) was dissolved in 40 ml of ethanol; 6 N hydrochloric acid (80 mL) was added; and the mixture was reacted at 80° C. for 16 h. After the reaction was completed, the reaction was concentrated under reduced pressure, and dichloromethane (80 mL), triethylamine (15.1 g, 0.15 mol) and BOC anhydride (21.8 g, 0.1 mol) were successively added to the residue. The resulting mixture was reacted at room temperature for 1 h. After the reaction was completed, the reaction solution was concentrated under reduced pressure and separated by silica gel column chromatography (EA/PE=5%-10%) to obtain compound 205C (19.1 g, 84%).

¹H NMR (400 MHz, CDCl₃) δ 4.20-4.12 (m, 2H), 3.46-3.34 (m, 1H), 2.97-2.81 (m, 2H), 1.88-1.77 (m, 2H), 1.76-1.62 (m, 2H), 1.45 (s, 9H).

Step 3:

### tert-butyl 4-[hydroxy-(2,3,4,5,6-pentadeuteriophenyl)methyl]piperidine-1-carboxylate (205D)

Tert-butyl 4-(2,3,4,5,6-pentadeuteriobenzoyl)piperidine-1-carboxylate (205C) (19.1 g, 65 mmol) was dissolved in methanol (50 mL), and sodium borohydride (1.51 g, 40 mmol) was slowly added in portions. After the addition was completed, the mixture was reacted at room temperature for 30 min and concentrated under reduced pressure. Dichloromethane (200 mL) was added to the residue, and the mixture was washed with saturated brine, dried over anhydrous sodium sulfate and concentrated under reduced pressure to obtain target compound, which was directly used in the subsequent reaction.

¹H NMR (400 MHz, CDCl₃) δ 4.39 (d, 1H), 4.19-4.12 (m, 1H), 4.08-4.01 (m, 1H), 2.70-2.62 (m, 1H), 2.62-2.52 (m, 1H), 2.00-1.90 (m, 1H), 1.79-1.70 (m, 2H), 1.44 (s, 9H), 1.33-1.22 (m, 2H).

Step 4:

### (1S,2S)-2-fluoro-N-[2-[2-[4-[(R)-(5-methyltetrazol-2-yl)-(2,3,4,5,6-pentadeuteriophenyl)methyl]piperidine-1-carbonyl]-4-pyridyl]-1,3-benzoxazol-5-yl]cyclopropanecarboxamide

205D was used as a raw material to obtain the corresponding compound 205 according to the synthetic method of compound 120.

LC-MS (ESI): m/z=586.3[M+H]⁺.

Compound 205: ¹H NMR (400 MHz, CDCl₃) δ 8.72 (t, 1H), 8.24 (s, 1H), 8.10-8.00 (m, 2H), 7.95 (s, 1H), 7.56-7.41 (m, 2H), 5.53 (d, 1H), 4.92-4.68 (m, 2H), 3.91 (d, 1H), 3.17-3.02 (m, 1H), 2.94-2.75 (m, 2H), 2.52 (d, 3H), 1.98-1.78 (m, 2H), 1.62-1.13 (m, 5H).

### Example 206 and Example 207

### (R/S)-(4-(5-(1-(difluoromethyl)-1H-imidazol-5-yl)benzo[d]oxazol-2-yl)pyridin-2-yl)(4-((5-(hydroxymethyl)-2H-tetrazol-2-yl)(phenyl)methyl)piperidin-1-yl)methanone and

### (R/S)-(4-(5-(1-(difluoromethyl)-1H-imidazol-4-yl)benzo[d]oxazol-2-yl)pyridin-2-yl)(4-((5-(hydroxymethyl)-2H-tetrazol-2-yl)(phenyl)methyl)piperidin-1-yl)methanone

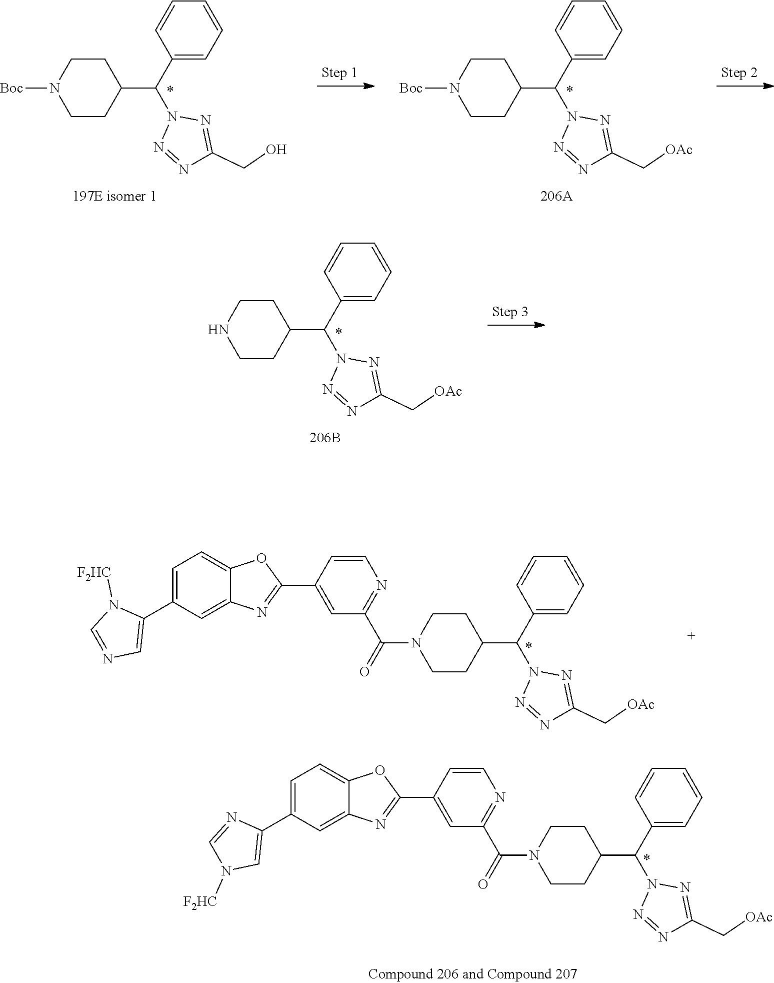

Step 1:

### tert-butyl-4-((5-(acetoxymethyl)-2H-tetrazol-2-yl)(phenyl)methyl)piperidine-1-carboxylate (206A)

Raw materials (197E, isomer 1 (750 mg, 2.0 mmol) and acetic anhydride (410 mg, 4.0 mmol)) were dissolved in 10 mL of dichloromethane at room temperature; then triethylamine (600 mg, 6.0 mmol) was added; and the mixture was stirred at room temperature for another 2 h. The reaction solution was subjected to rotary evaporation and then directly subjected to silica gel column chromatography (PE:EA=10:1) to obtain compound 206A (550 mg, yield: 660%).

LC-MS (ESI): m/z=415.2 [M+H]⁺.

Step 2:

### (2-(phenyl(piperidin-4-yl)methyl)-2H-tetrazol-5-yl)methyl acetate (206B)

Compound 206A (550 mg, 1.3 mmol) was dissolved in dichloromethane (6 mL) at room temperature, and then trifluoroacetic acid (2 mL) was added. The mixture was stirred at room temperature for another 2 h. The reaction solution was subjected to rotary evaporation, and then 10 mL of saturated sodium bicarbonate was added. The mixture was extracted with dichloromethane (5 mL×3), and the organic phase was combined, washed with saturated sodium chloride solution (10 mL×1), dried over anhydrous sulfuric acid, filtered and concentrated under reduced pressure to obtain compound 206B (300 mg, yield: 74%), which was directly used in the next reaction without purification.

LC-MS (ESI): m/z=315.2 [M+H]⁺.

Step 3:

### (R/S)-(4-(5-(1-(difluoromethyl)-1H-imidazol-4-yl)benzo[d]oxazol-2-yl)pyridin-2-yl)(4-((5-(hydroxymethyl)-2H-tetrazol-2-yl)(phenyl)methyl)piperidin-1-yl)methanone and (R/S)-(4-(5-(1-(difluoromethyl)-1H-imidazol-5-yl)benzo[d]oxazol-2-yl)pyridin-2-yl)(4-((5-(hydroxymethyl)-2H-tetrazol-2-yl)(phenyl)methyl)piperidin-1-yl)methanone (Compound 206 and Compound 207)

According to the synthetic method of compound 170, the compounds were obtained by using a mixture of compound 170D and compound 171D as a raw material, condensing with compound 206B and using prep-HPLC, wherein the preparation conditions were as follows:

instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 18 min; retention time for compound 1: 14.3 min; retention time for compound 2: 16.8 min.

LC-MS (ESI): m/z=654.2 [M+H]⁺.

Compound 206: 1H NMR (400 MHz, CD₃OD) δ 8.82 (t, 1H), 8.35 (s, 1H), 8.26-8.23 (m, 2H), 7.96 (d, 1H), 7.89-7.87 (m, 1H), 7.67-7.52 (m, 4H), 7.43-7.35 (m, 3H), 7.23 (d, 1H), 5.86-5.84 (m, 1H), 5.36-5.32 (m, 2H), 4.68-4.65 (m, 1H), 3.83-3.81 (m, 1H), 3.21-3.14 (m, 1H), 3.01-2.95 (m, 2H), 2.11-2.06 (m, 3H), 1.55-1.37 (m, 4H).

Compound 207: 1H NMR (400 MHz, CD₃OD) δ 8.81 (t, 1H), 8.33 (s, 1H), 8.26-8.23 (m, 2H), 8.17 (d, 1H), 7.99-7.96 (m, 2H), 7.88-7.75 (m, 1H), 7.64-7.58 (m, 3H), 7.45-7.34 (m, 3H), 5.87-5.84 (m, 1H), 5.37-5.32 (m, 2H), 4.71-4.67 (m, 1H), 3.18-3.13 (m, 1H), 3.01-2.95 (m, 2H), 2.11-2.06 (m, 3H), 1.55-1.38 (m, 4H), 1.21-1.17 (m, 1H).

### Example 208 and Example 209

### (1S,2S)-2-fluoro-N-(2-(2-(3-((R/S)-(5-methyl-2H-tetrazol-2-yl)(phenyl)methyl)-8-azabicyclo[3.2.1]octane-8-carbonyl)pyridin-4-yl)benzo[d]oxazol-5-yl)cyclopropanecarboxamide and

### (1S,2S)-2-fluoro-N-(2-(2-(3-((S/R)-(5-methyl-2H-tetrazol-2-yl)(phenyl)methyl)-8-azabicyclo[3.2.1]octane-8-carbonyl)pyridin-4-yl)benzo[d]oxazol-5-yl)cyclopropanecarboxamide

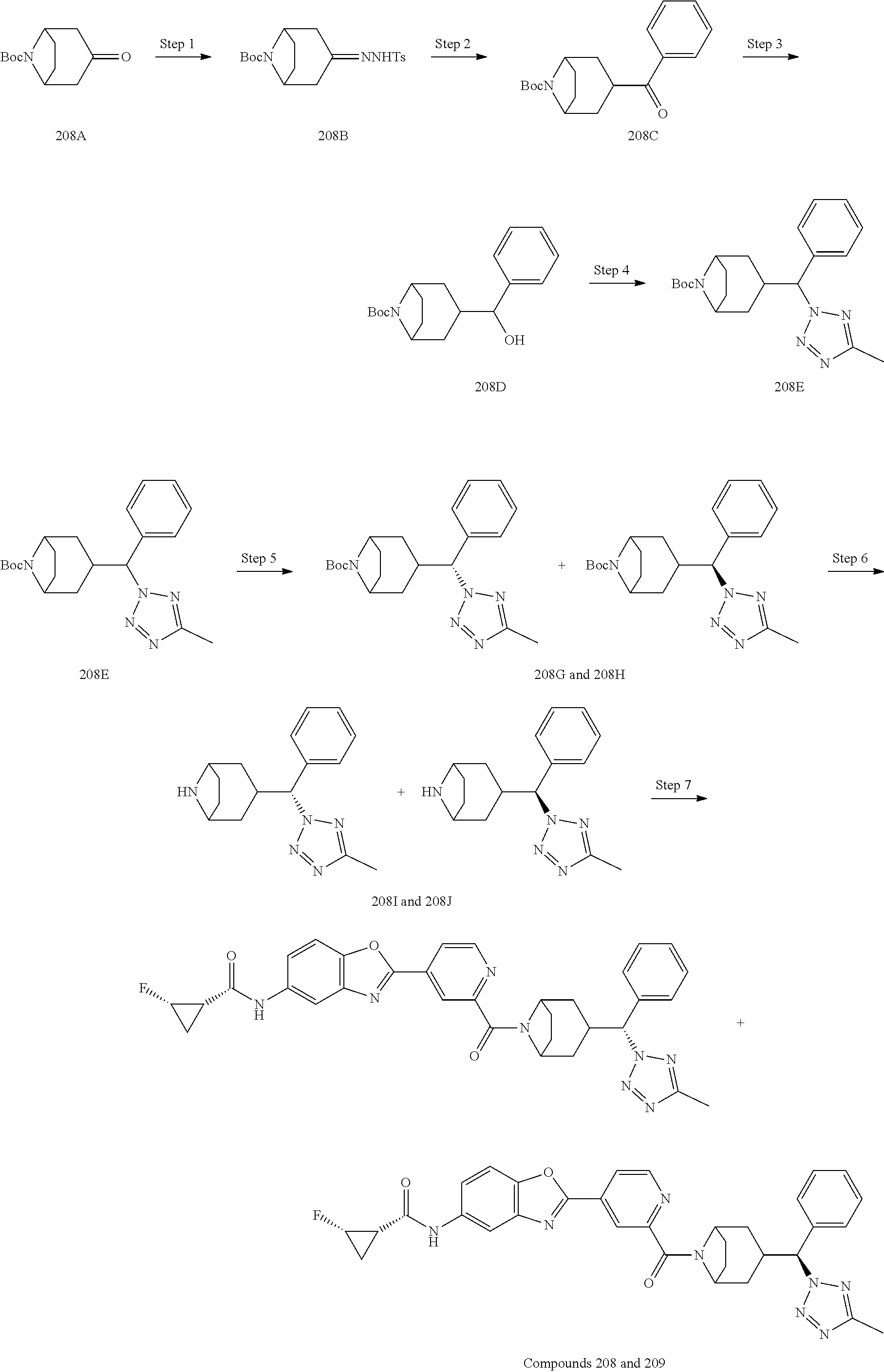

Compound 208A N-tert-butoxycarbonyl-nortropinone was used as a raw material to synthesize compound 208E according to the synthetic method of compound 202.

LC-MS (ESI): m/z=200.2 [M−183]⁺ (carbon positive ion peak removing Boc and tetrazole).

Step 5:

### tert-butyl 3-((R/S)-(5-methyl-2H-tetrazol-2-yl)(phenyl)methyl)-8-azabicyclo[3.2.1]octane-8-carboxylate and

### tert-butyl 3-((S/R)-(5-methyl-2H-tetrazol-2-yl)(phenyl)methyl)-8-azabicyclo[3.2.1]octane-8-carboxylate (208G and 208H)

Compound 208E (950 mg) was resolved by SFC to obtain compound 208G (425 mg) and compound 208H (519 mg).

SFC resolution conditions were as follows:

instrument: MG II preparative SFC (SFC-14); column type: ChiralCel OJ, 250×30 mm I.D., 5 μm; the samples were dissolved in about 20 ml of methanol/DCM; mobile phase: A for CO₂ and B for isopropanol; gradient: mobile phase B: 20%; flow rate: 60 mL/min; column pressure: 100 bar; column temperature: 38° C.; wavelength: 220 nm; elution time: 4 min; retention time: 2.801 min for compound 208G and 3.338 min for compound 208H; sample injection: 1.0 mL each time.

Compounds 208G and 208H were used as raw materials to obtain compounds 208 and 209 according to the synthetic method of compound 202.

Compound 208: 1H NMR (400 MHz, CDCl₃) δ 8.72-8.70 (m, 1H), 8.46-8.44 (m, 1H), 8.33-8.27 (m, 1H), 8.08-8.06 (m, 1H), 7.96 (s, 1H), 7.55-7.31 (m, 7H), 5.50-5.48 (m, 1H), 4.90-4.85 (m, 1.5H), 4.73-4.69 (m, 1.5H), 3.29-3.15 (m, 1H), 2.53-2.50 (m, 3H), 1.92-1.64 (m, 8H), 1.40-1.19 (m, 3H).

LC-MS (ESI): m/z=607.3 [M+H]⁺.

Compound 209: 1H NMR (400 MHz, CDCl₃) δ 8.72-8.70 (m, 1H), 8.46-8.43 (m, 1H), 8.37-8.29 (m, 1H), 8.08-8.05 (m, 1H), 7.96 (s, 1H), 7.55-7.31 (m, 7H), 5.51-5.48 (m, 1H), 4.90-4.86 (m, 1.5H), 4.70-4.68 (m, 1.5H), 3.28-3.15 (m, 1H), 2.53-2.50 (m, 3H), 1.92-1.63 (m, 8H), 1.40-1.18 (m, 3H).

LC-MS (ESI): m/z=607.3 [M+H]⁺.

### Example 210

### (1S,2S)—N-(2-(2-(4-((R/S)-(5-(aminomethyl)-2H-tetrazol-2-yl)(phenyl)methyl)piperidine-1-carbonyl)pyridin-4-yl)benzo[d]oxazol-5-yl)-2-fluorocyclopropane-1-carboxamide

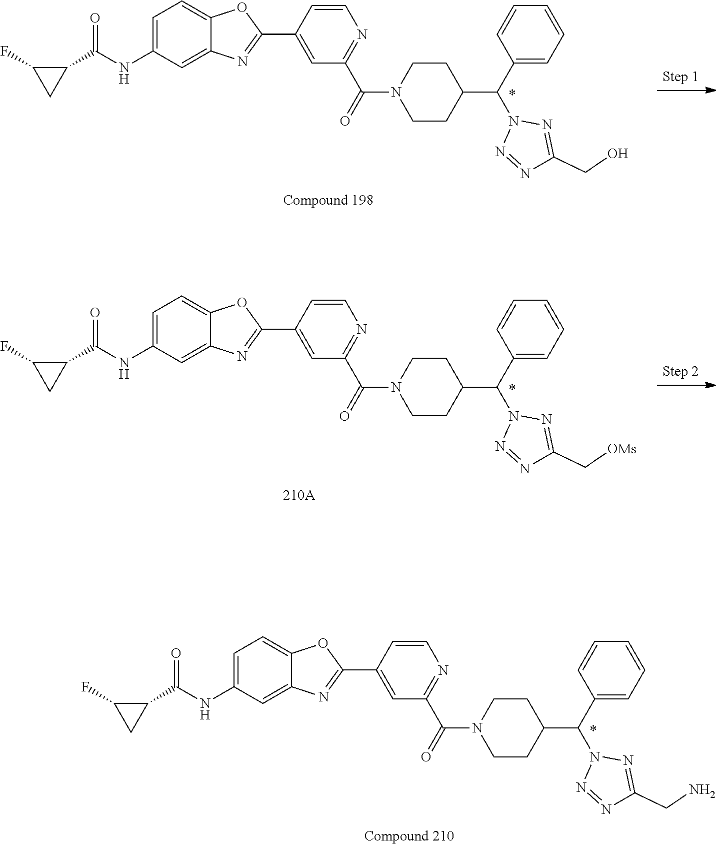 [SMILES: O=C(Nc1ccc2oc(-c3ccnc(C(=O)N4CCC(C(c5ccccc5)n5nnc(CO)n5)CC4)c3)nc2c1)[C@@H]1C[C@@H]1F ; CS(=O)(=O)OCc1nnn(C(c2ccccc2)C2CCN(C(=O)c3cc(-c4nc5cc(NC(=O)[C@@H]6C[C@@H]6F)ccc5o4)ccn3)CC2)n1 ; NCc1nnn(C(c2ccccc2)C2CCN(C(=O)c3cc(-c4nc5cc(NC(=O)[C@@H]6C[C@@H]6F)ccc5o4)ccn3)CC2)n1]

Step 1:

### (2-((R/S)-(1-(4-(5-((1S,2S)-2-fluorocyclopropane-1-carboxamido)benzo[d]oxazol-2-yl)picolinoyl)piperidin-4-yl)(phenyl)methyl)-2H-tetrazol-5-yl)methyl Methanesulfonate (210A)

Compound 198 (200 mg, 0.33 mmol) was dissolved in dichloromethane (10 mL), and triethylamine (66 mg, 0.66 mmol) was added. The mixture was cooled to about 0° C. in an ice-water bath, and methyl sulfonyl chloride (45.6 mg, 0.40 mmol) was added dropwise. After completion of the dropwise addition, the reaction was allowed to naturally warm to room temperature and stirred for 2 hours, and the reaction solution was diluted with dichloromethane (20 ml), washed with water and saturated brine, dried over anhydrous sodium sulfate and concentrated to obtain light yellow viscous title compound 210A (180 mg, yield: 81.0%).

LCMS m/z=675.2 [M+1]*.

Step 2:

### (1S,2S)—N-(2-(2-(4-((R/S)-(5-(aminomethyl)-2H-tetrazol-2-yl)(phenyl)methyl)piperidine-1-carbonyl)pyridin-4-yl)benzo[d]oxazol-5-yl)-2-fluorocyclopropane-1-carboxamide (Compound 210)

Compound 210A (90 mg, 0.133 mmol) was dissolved in acetonitrile (5 mL), and ammonia water (25%, 2 ml) was added. The reaction was stirred at room temperature for 16 hours. The reaction solution was diluted with water (20 ml) and extracted with ethyl acetate (10 ml×3). The organic phase was combined, washed with water (10 mL×2), dried over anhydrous sodium sulfate and concentrated. The residue was separated and purified by silica gel column chromatograph (dichloromethane: methanol (v/v)=1:0-10:1) to obtain title compound 210 as a white solid (30 mg, yield: 37.9%).

¹H NMR (400 MHz, d-DMSO) δ 10.67 (s, 1H), 8.81-8.80 (m, 1H), 8.25 (d, 1H), 8.17-8.16 (m, 1H), 8.15-8.13 (m, 1H), 7.81-7.78 (d, 1H), 7.66-7.57 (m, 3H), 7.45-7.33 (m, 3H), 6.00-5.95 (m, 1H), 5.05-5.01 (m, 0.5H), 4.89-4.86 (m, 0.5H), 4.55-4.45 (m, 1H), 4.05-3.96 (m, 3H), 3.11-3.06 (m, 1H), 2.90-2.81 (m, 2H), 2.10-2.05 (m, 1H), 1.72-1.62 (m, 1H), 1.40-1.35 (m, 3H), 1.30-1.15 (m, 3H), 1.06-1.02 (m, 1H).

LCMS m/z=596.2 [M+1]+.

### Example 211

### (1S,2S)-2-fluoro-N-(2-(2-(4-((R/S)-(5-((methylamino)methyl)-2H-tetrazol-2-yl)(phenyl)methyl)piperidine-1-carbonyl)pyridin-4-yl)benzo[d]oxazol-5-yl)cyclopropane-1-carboxamide

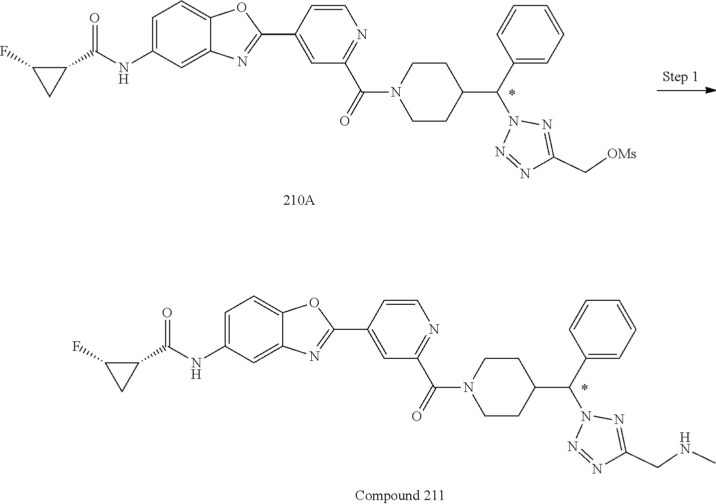 [SMILES: CS(=O)(=O)OCc1nnn(C(c2ccccc2)C2CCN(C(=O)c3cc(-c4nc5cc(NC(=O)[C@@H]6C[C@@H]6F)ccc5o4)ccn3)CC2)n1 ; CNCc1nnn(C(c2ccccc2)C2CCN(C(=O)c3cc(-c4nc5cc(NC(=O)[C@@H]6C[C@@H]6F)ccc5o4)ccn3)CC2)n1]

Step 1:

### (1S,2S)-2-fluoro-N-(2-(2-(4-((R/S)-(5-((methylamino)methyl)-2H-tetrazol-2-yl)(phenyl)methyl)piperidine-1-carbonyl)pyridin-4-yl)benzo[d]oxazol-5-yl)cyclopropane-1-carboxamide (Compound 211)

Compound 210A (90 mg, 0.133 mmol) was dissolved in acetonitrile (5 mL), and a solution of methylamine in dioxane (2 mol/L, 1 ml) was added. The reaction was stirred at room temperature for 2 hours. The reaction solution was diluted with water (20 ml) and extracted with ethyl acetate (10 ml×3). The organic phase was combined, washed with water (10 mL×2), dried over anhydrous sodium sulfate and concentrated. The residue was separated and purified by silica gel column chromatograph (dichloromethane: methanol (v/v)=1:0-10:1) to obtain title compound 211 as a white solid (45 mg, yield: 56.2%).

¹H NMR (400 MHz, d-DMSO) δ 10.48 (s, 1H), 8.82-8.80 (m, 1H), 8.24 (d, 1H), 8.17-8.16 (m, 1H), 8.15-8.12 (m, 1H), 7.81-7.78 (d, 1H), 7.66-7.57 (m, 3H), 7.45-7.30 (m, 3H), 6.00-5.95 (m, 1H), 5.05-5.01 (m, 0.5H), 4.89-4.86 (m, 0.5H), 4.51-4.45 (m, 1H), 3.88-3.83 (m, 3H), 3.11-3.06 (m, 1H), 2.90-2.81 (m, 2H), 2.28-2.23 (m, 3H), 2.05-2.00 (m, 1H), 1.72-1.62 (m, 1H), 1.40-1.35 (m, 3H), 1.25-1.15 (m, 3H), 1.05-1.01 (m, 1H).

LCMS m/z=610.2 [M+1]+.

### Examples 212 and 213

### (1S,2S)-2-fluoro-N-(2-(2-(4-((R/S)(2-(2-hydroxyethyl)-2H-tetrazol-5-yl)(phenyl)methyl)piperazine-1-carbonyl)pyridin-4-yl)benzo[d]oxazol-5-yl)cyclopropane-1-carboxamide and

### (1S,2S)-2-fluoro-N-(2-(2-(4-((S/R)(2-(2-hydroxyethyl)-2H-tetrazol-5-yl)(phenyl)methyl)piperazine-1-carbonyl)pyridin-4-yl)benzo[d]oxazol-5-yl)cyclopropane-1-carboxamide

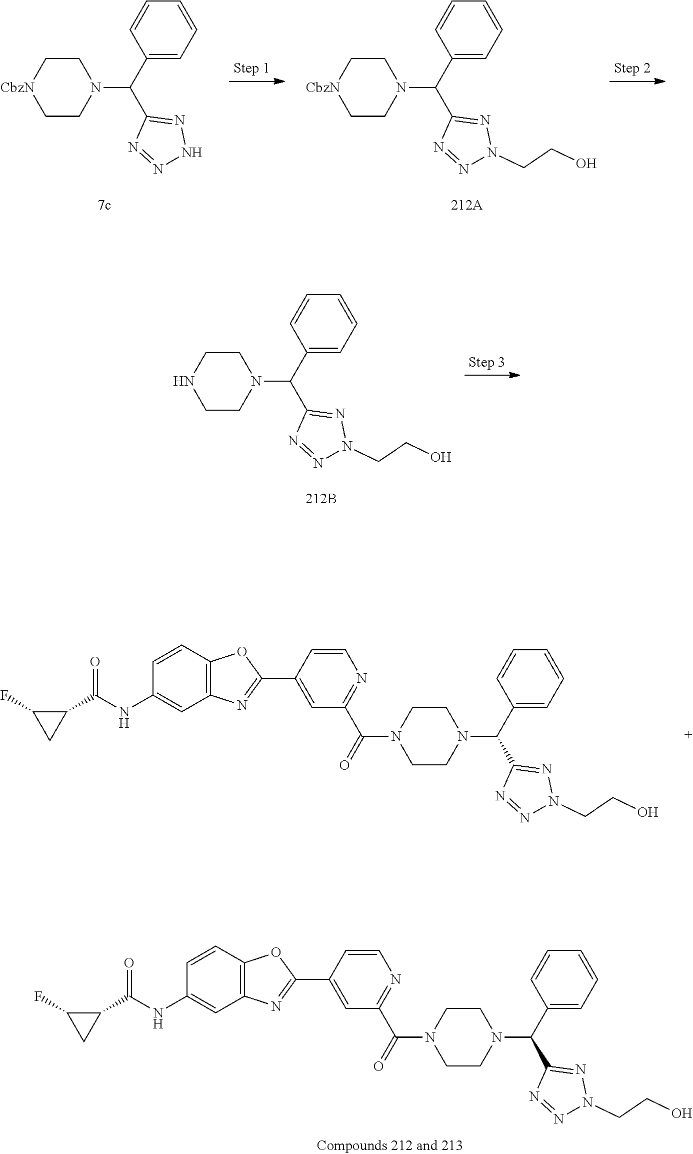 [SMILES: O=C(OCc1ccccc1)N1CCN(C(c2ccccc2)c2nn[nH]n2)CC1 ; O=C(OCc1ccccc1)N1CCN(C(c2ccccc2)c2nnn(CCO)n2)CC1 ; OCCn1nnc(C(c2ccccc2)N2CCNCC2)n1 ; O=C(Nc1ccc2oc(-c3ccnc(C(=O)N4CCN([C@H](c5ccccc5)c5nnn(CCO)n5)CC4)c3)nc2c1)[C@@H]1C[C@@H]1F ; O=C(Nc1ccc2oc(-c3ccnc(C(=O)N4CCN([C@@H](c5ccccc5)c5nnn(CCO)n5)CC4)c3)nc2c1)[C@@H]1C[C@@H]1F]

Step 1:

### Benzyl 4-((2-(2-hydroxyethyl)-2H-tetrazol-5-yl)(phenyl)methyl)piperazine-1-carboxylate (212A)

7c (6 g, 15.8 mol) and potassium carbonate (2.2 g, 15.8 mol) were added to DMF (120 ml), and then 2-bromomethane-1-ol (3.0 g, 23.7 mol) was added. The reaction was warmed to 80° C., stirred for 16 hours and then cooled to room temperature. The reaction solution was poured into water, extracted twice with ethyl acetate and dried over anhydrous sodium sulfate. The organic phase was concentrated, and the residue was separated and purified by HPLC to obtain compound 212A (2.5 g, 37%). Preparation method: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 20 min; retention time: 12.65 min.

LC-MS (ESI): m/z=423.3[M+H]⁺.

Step 2:

### 2-(5-(phenyl(piperazin-1-yl)methyl)-2H-tetrazol-2-yl)ethan-1-ol (212B)

Compound 212A (1.3 g, 3.0 mmol) was added to ethanol (13 ml) and water (2 ml), and then potassium hydroxide (6.9 g, 120 mmol) was added. The reaction was warmed to 80° C. and stirred for 16 hours. When the reaction solution was concentrated to dryness, the reaction solution was extracted with ethyl acetate, dried over anhydrous sodium sulfate and concentrated to obtain a crude product of 212B, which was directly used in the next reaction.

LC-MS (ESI): m/z=289.2[M+H]⁺.

Step 3:

### (1S,2S)-2-fluoro-N-(2-(2-(4-((R/S)-(2-(2-hydroxyethyl)-2H-tetrazol-5-yl)(phenyl)methyl)piperazine-1-carbonyl)pyridin-4-yl)benzo[d]oxazol-5-yl)cyclopropane-1-carboxamide and

### (1S,2S)-2-fluoro-N-(2-(2-(4-((S/R)-(2-(2-hydroxyethyl)-2H-tetrazol-5-yl)(phenyl)methyl)piperazine-1-carbonyl)pyridin-4-yl)benzo[d]oxazol-5-yl)cyclopropane-1-carboxamide (compound 212 and compound 213)

Intermediate 117F (450 mg, 1.3 mmol) was dissolved in N,N-dimethylformamide (5 mL), and the mixture was cooled to 0° C.; HATU (550 mg, 1.45 mmol) and DIPEA (500 mg, 3.9 mmol) were added; and then 212B (750 mg, 2.6 mmol) was added. After the addition, the mixture was stirred at room temperature for two hours and then extracted twice with water and ethyl acetate. The organic phase was combined, dried over anhydrous sodium sulfate and concentrated to dryness under reduced pressure, and the residue was separated by prep-HPLC and then subjected to chiral separation to obtain compound 212 (200 mg, 25%) and compound 213 (190 mg, 24%).

HPLC preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 20% to 70%; flow rate: 15 ml/min; elution time: 18 min; retention time: 13.68 min. Chiral resolution conditions: instrument: Waters UPC2 analytical SFC (SFC-H); chromatographic column: ChiralPak AD, 150×4.6 mm, inner diameter: 3 μm; mobile phase: A: CO₂, B: ethanol (0.05% DEA); gradient: b5-40%; flow rate: 2.5 ml/min; back pressure: 100 bar; column temperature: 35° C.; wavelength: 220 nm; retention time for compound 212: 7.023 min; retention time for compound 213: 9.907 min.

Compound 212:

LC-MS (ESI): m/z=612.3 [M+H]⁺.

¹H NMR (400 MHz, MeOD) δ 8.76-8.75 (m, 1H), 8.28-8.27 (m, 1H), 8.17-8.15 (m, 2H), 7.70-7.62 (m, 1H), 7.61-7.49 (m, 3H), 7.38-7.23 (m, 3H), 5.04 (s, 1H), 4.93-4.77 (m, 1H), 4.76-4.71 (m, 2H), 4.09-4.03 (m, 2H), 3.85-3.83 (t, 2H), 3.58-3.56 (t, 2H), 2.70-2.66 (m, 1H), 2.58-2.52 (m, 2H), 2.46-2.42 (m, 1H), 2.02-1.97 (m, 1H), 1.85-1.71 (m, 1H), 1.21-1.14 (m, 1H).

Compound 213:

LC-MS (ESI): m/z=612.3 [M+H]⁺.

¹H NMR (400 MHz, MeOD) δ 8.82-8.74 (m, 1H), 8.32-8.30 (m, 1H), 8.23-8.16 (m, 2H), 7.67-7.59 (m, 2H), 7.54-7.53 (m, 2H), 7.36-7.27 (m, 3H), 5.04 (s, 1H), 4.96-4.89 (m, 1H), 4.76-4.71 (m, 2H), 4.09-4.06 (t, 2H), 3.86-3.84 (t, 2H), 3.60-3.58 (t, 2H), 2.70-2.67 (m, 1H), 2.58-2.57 (m, 2H), 2.46-2.43 (m, 1H), 2.06-1.99 (m, 1H), 1.86-1.76 (m, 1H), 1.23-1.14 (m, 1H).

### Examples 214 and 215

### (1S,2S)-2-fluoro-N-(2-(2-(4-((R/S)(2-(methoxymethyl)-2H-tetrazol-5-yl)(phenyl)methyl)piperazine-1-carbonyl)pyridin-4-yl)benzo[d]oxazol-5-yl)cyclopropane-1-carboxamide and

### (1S,2S)-2-fluoro-N-(2-(2-(4-((S/R)(2-(methoxymethyl)-2H-tetrazol-5-yl)(phenyl)methyl)piperazine-1-carbonyl)pyridin-4-yl)benzo[d]oxazol-5-yl)cyclopropane-1-carboxamide

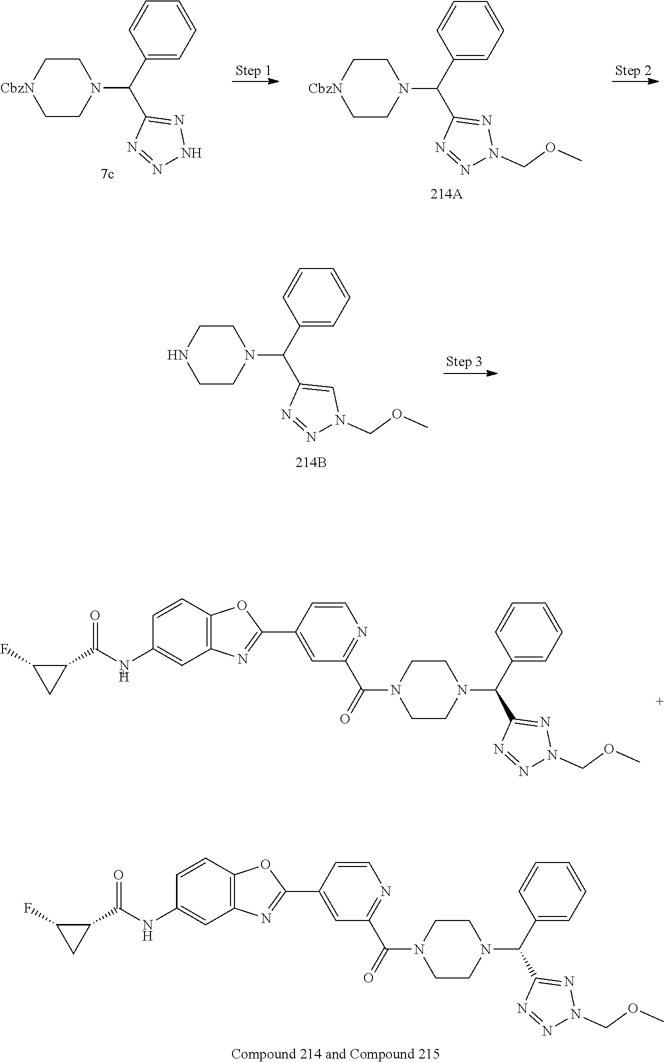 [SMILES: O=C(OCc1ccccc1)N1CCN(C(c2ccccc2)c2nn[nH]n2)CC1 ; COCn1nnc(C(c2ccccc2)N2CCN(C(=O)OCc3ccccc3)CC2)n1 ; COCn1cc(C(c2ccccc2)N2CCNCC2)nn1 ; COCn1nnc([C@H](c2ccccc2)N2CCN(C(=O)c3cc(-c4nc5cc(NC(=O)[C@@H]6C[C@@H]6F)ccc5o4)ccn3)CC2)n1 ; COCn1nnc([C@@H](c2ccccc2)N2CCN(C(=O)c3cc(-c4nc5cc(NC(=O)[C@@H]6C[C@@H]6F)ccc5o4)ccn3)CC2)n1]

Step 1:

### Benzyl 4-((2-(methoxymethyl)-2H-tetrazol-5-yl)(phenyl)methyl)piperazine-1-carboxylate (214A)

7c (4.5 g, 12.0 mmol) and sodium hydroxide (1.44 g, 36.0 mmol) were added to DMF (90 ml), and then bromo(methoxy)methane (3.0 g, 24 mmol) was added. The reaction was stirred at room temperature for 5 hours, and then cooled to room temperature, and the reaction solution was poured into water, extracted twice with ethyl acetate and dried over anhydrous sodium sulfate. The organic phase was concentrated, and the residue was separated and purified by HPLC to obtain compound 214A (1.4 g, 28%). Preparation method: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 40% to 70%; flow rate: 15 ml/min; elution time: 20 min; retention time: 15.82 min.

LC-MS (ESI): m/z=423.3 [M+H]⁺.

Step 2:

### 1-((2-(methoxymethyl)-2H-tetrazol-5-yl)(phenyl)methyl)piperazine (214B)

Compound 214A (1.3 g, 3.0 mmol) was added to ethanol (13 ml) and water (2 ml), and then potassium hydroxide (6.9 g, 120 mmol) was added. The reaction was warmed to 80° C. and stirred for 16 hours. When the reaction solution was concentrated to dryness, the reaction solution was extracted with ethyl acetate, dried over anhydrous sodium sulfate and concentrated to obtain a crude product of 214B, which was directly used in the next reaction.

LC-MS (ESI): m/z=289.2[M+H]⁺.

Step 3:

### (1S,2S)-2-fluoro-N-(2-(2-(4-((R/S)(2-(methoxymethyl)-2H-tetrazol-5-yl)(phenyl)methyl)piperazine-1-carbonyl)pyridin-4-yl)benzo[d]oxazol-5-yl)cyclopropane-1-carboxamide and

### (1S,2S)-2-fluoro-N-(2-(2-(4-((S/R)(2-(methoxymethyl)-2H-tetrazol-5-yl)(phenyl)methyl)piperazine-1-carbonyl)pyridin-4-yl)benzo[d]oxazol-5-yl)cyclopropane-1-carboxamide (Compound 214 and Compound 215)

Intermediate 117F (450 mg, 1.3 mmol) was dissolved in N,N-dimethylformamide (5 mL), and the mixture was cooled to 0° C.; HATU (550 mg, 1.45 mmol) and DIPEA (500 mg, 3.9 mmol) were added; and then 200B (750 mg, 2.6 mmol) was added. After the addition, the mixture was stirred at room temperature for two hours and then extracted twice with water and ethyl acetate. The organic phase was combined, dried over anhydrous sodium sulfate and concentrated to dryness under reduced pressure, and the residue was separated by prep-HPLC and then subjected to chiral separation to obtain compound 214 (130 mg, 16%) and compound 215 (150 mg, 19%). HPLC preparation conditions: instrument: waters 2767 (preparative liquid phase chromatographic instrument); chromatographic column: SunFire@ Prep C18 (19 mm×250 mm). The sample was dissolved in DMF and filtered with a 0.45 m filter to prepare a sample solution. Preparative chromatography conditions: mobile phases A and B: mobile phase A: acetonitrile; mobile phase B: water (containing 5 mM ammonium acetate); gradient elution: the content of mobile phase A rises from 20% to 70%; flow rate: 15 ml/min; elution time: 18 min; retention time: 15.24 min. Chiral resolution conditions: instrument: Waters UPC2 analytical SFC(SFC-H); chromatographic column: ChiralCel OJ, 150×4.6 mm, inner diameter: 3 μm; mobile phase: A: CO₂, B: methanol (0.05% DEA); gradient: b5-40%; flow rate: 2.5 ml/min; back pressure: 100 bar; column temperature: 35° C.; wavelength: 220 nm; cycle time: about 4 min. retention time for compound 214: 5.919 min; retention time for compound 215: 6.298 min.

Compound 214:

LC-MS (ESI): m/z=612.3 [M+H]⁺.

¹H NMR (400 MHz, MeOD) δ 8.77-8.76 (m, 1H), 8.28-8.27 (m, 1H), 8.17-8.16 (m, 2H), 7.66-7.64 (m, 1H), 7.58-7.52 (m, 3H), 7.37-7.27 (m, 3H), 5.89 (s, 1H), 5.12 (s, 1H), 4.93-4.77 (m, 1H), 3.86-3.83 (t, 2H), 3.60-3.57 (t, 2H), 3.43 (s, 3H), 2.73-2.70 (m, 1H), 2.62-2.55 (m, 2H), 2.47-2.44 (m, 1H), 2.04-1.97 (m, 1H), 1.85-1.71 (m, 1H), 1.22-1.14 (m, 1H).

Compound 215:

LC-MS (ESI): m/z=612.3 [M+H]⁺.

¹H NMR (400 MHz, MeOD) δ 8.78-8.77 (m, 1H), 8.31-8.30 (m, 1H), 8.19-8.18 (m, 2H), 7.67-7.64 (m, 2H), 7.54-7.52 (m, 2H), 7.37-7.27 (m, 3H), 5.89 (s, 2H), 5.10 (s, 1H), 4.92-4.74 (m, 1H), 3.87-3.85 (t, 2H), 3.61-3.59 (t, 2H), 3.44 (s, 3H), 2.74-2.68 (m, 1H), 2.63-2.54 (m, 2H), 2.47-2.42 (m, 1H), 2.04-1.98 (m, 1H), 1.86-1.76 (m, 1H), 1.24-1.13 (m, 1H).

### Example 216 and Example 217

### (4-(5-(1-(difluoromethyl)-1H-imidazol-5-yl)benzo[d]oxazol-2-yl)pyridin-2-yl)(4-((5-(hydroxymethyl)-2H-tetrazol-2-yl)(phenyl)methyl)piperidin-1-yl)methanone and

### (4-(5-(1-(difluoromethyl)-1H-imidazol-4-yl)benzo[d]oxazol-2-yl)pyridin-2-yl)(4-((5-(hydroxymethyl)-2H-tetrazol-2-yl)(phenyl)methyl)piperidin-1-yl)methanone

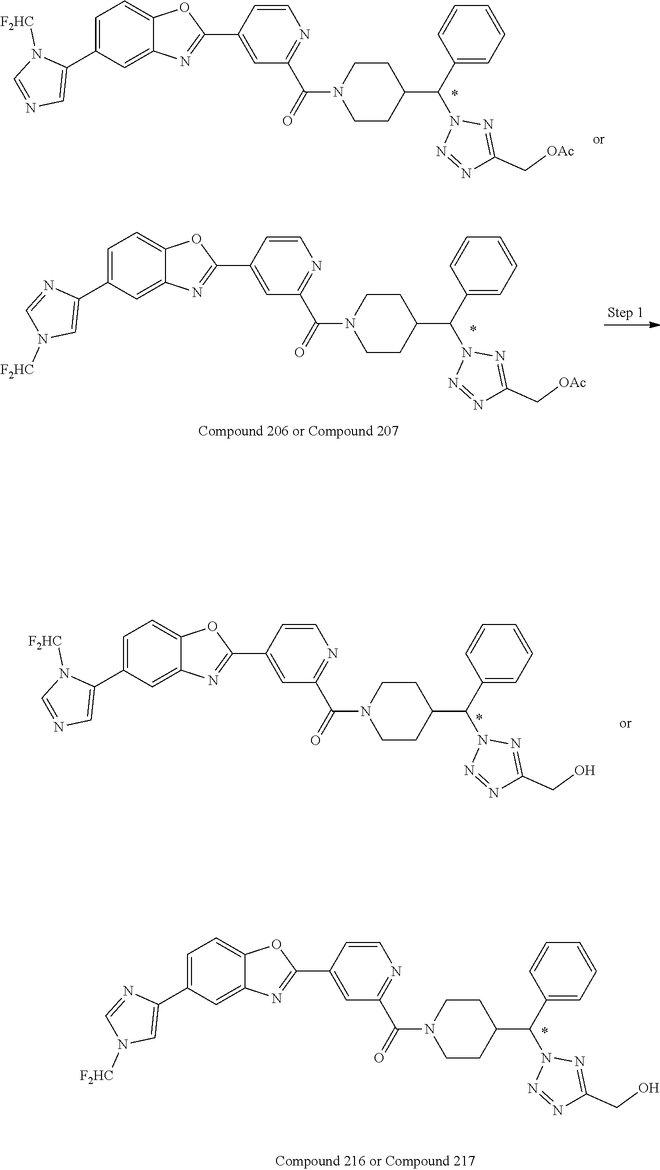 [SMILES: CC(=O)OCc1nnn(C(c2ccccc2)C2CCN(C(=O)c3cc(-c4nc5cc(-c6cncn6C(F)F)ccc5o4)ccn3)CC2)n1 ; CC(=O)OCc1nnn(C(c2ccccc2)C2CCN(C(=O)c3cc(-c4nc5cc(-c6cn(C(F)F)cn6)ccc5o4)ccn3)CC2)n1 ; O=C(c1cc(-c2nc3cc(-c4cncn4C(F)F)ccc3o2)ccn1)N1CCC(C(c2ccccc2)n2nnc(CO)n2)CC1 ; O=C(c1cc(-c2nc3cc(-c4cn(C(F)F)cn4)ccc3o2)ccn1)N1CCC(C(c2ccccc2)n2nnc(CO)n2)CC1]

Step 1:

### (4-(5-(1-(difluoromethyl)-1H-imidazol-5-yl)benzo[d]oxazol-2-yl)pyridin-2-yl)(4-((5-(hydroxymethyl)-2H-tetrazol-2-yl)(phenyl)methyl)piperidin-1-yl)methanone and

### (4-(5-(1-(difluoromethyl)-1H-imidazol-4-yl)benzo[d]oxazol-2-yl)pyridin-2-yl)(4-((5-(hydroxymethyl)-2H-tetrazol-2-yl)(phenyl)methyl)piperidin-1-yl)methanone

Compound 206 (65 mg, 0.1 mmol) was dissolved in methanol (5 mL) at room temperature; potassium carbonate (28 mg, 0.2 mmol) was added; and the mixture was stirred at room temperature for another 1 h. The reaction solution was subjected to rotary evaporation and then directly purified by silica gel column chromatography (DCM:MeOH=20:1) to obtain compound 216 (30 mg, yield: 49%).

LC-MS (ESI): m/z=612.3 [M+H]⁺.

¹H NMR (400 MHz, CDCl₃) δ 8.78-8.76 (m, 1H), 8.43 (s, 1H), 8.18 (d, 1H), 8.14-8.12 (m, 1H), 7.88 (s, 1H), 7.74 (d, 1H), 7.56-7.49 (m, 3H), 7.41-7.34 (m, 3H), 7.24 (s, 1H), 7.05 (t, 1H), 5.59 (d, 1H), 4.97-4.92 (m, 2H), 4.78-4.76 (m, 1H), 4.02-4.00 (m, 1H), 3.15-3.05 (m, 1H), 2.93-2.78 (m, 2H), 2.09-1.96 (m, 1H), 1.61-1.22 (m, 4H).

Compound 207 was used as a raw material to obtain compound 217 according to the synthetic method of compound 216.

LC-MS (ESI): m/z=612.2 [M+H]⁺.

¹H NMR (400 MHz, CDCl₃) δ 7.75-8.73 (m, 1H), 8.41 (s, 1H), 8.22 (d, 1H), 8.43-8.11 (m, 1H), 7.94-7.92 (m, 1H), 7.89 (d, 1H), 7.66-7.63 (m, 1H), 7.60-7.52 (m, 3H), 7.41-7.34 (m, 3H), 7.13 (t, 1H), 5.58 (d, 1H), 4.98-4.91 (m, 2H), 4.78-4.76 (m, 1H), 4.01-3.98 (m, 1H), 3.14-3.07 (m, 1H), 2.93-2.78 (m, 2H), 2.22-2.14 (m, 1H), 1.55-1.25 (m, 4H).

### Example 218

### (1S,2S)-2-fluoro-N-(2-(3-methoxy-2-(4-((R/S)-(5-methyl-2H-tetrazol-2-yl)(phenyl)methyl)piperidine-1-carbonyl)pyridin-4-yl)benzo[d]oxazol-5-yl)cyclopropane-1-carboxamide

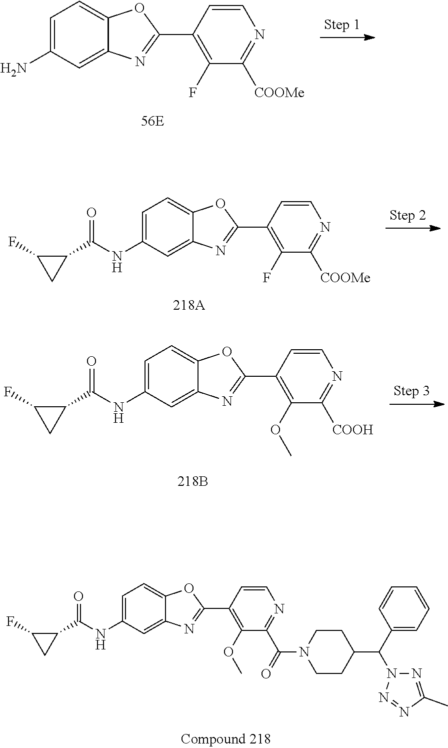 [SMILES: COC(=O)c1nccc(-c2nc3cc(N)ccc3o2)c1F ; COC(=O)c1nccc(-c2nc3cc(NC(=O)[C@@H]4C[C@@H]4F)ccc3o2)c1F ; COc1c(-c2nc3cc(NC(=O)[C@@H]4C[C@@H]4F)ccc3o2)ccnc1C(=O)O ; COc1c(-c2nc3cc(NC(=O)[C@@H]4C[C@@H]4F)ccc3o2)ccnc1C(=O)N1CCC(C(c2ccccc2)n2nnc(C)n2)CC1]

Step 1:

### Methyl-3-fluoro-4-(5-((1S,2S)-2-fluorocyclopropane-1-carboxamido)benzo[d]oxazol-2-yl)picolinate (218A)

(1S,2S)-2-fluorocycloprop-1-carboxylic acid (66 mg, 0.63 mmol), DMF (6 mL), DIPEA (155 mg, 1.2 mmol), HATU (228 mg, 0.6 mmol) and compound 56E (170 mg, 0.6 mmol) were successively added to a single-necked flask, and the mixture was stirred at room temperature for 3 h. The reaction was quenched by adding saturated aqueous sodium bicarbonate solution (30 mL), extracted with dichloromethane (50 mL×1) and allowed to stand for phase separation. The aqueous phase was washed with dichloromethane (100 mL×2). The combined organic phase was dried over anhydrous sodium sulfate and concentrated under reduced pressure to obtain a crude product from the residue, and the crude product was separated by column chromatography (eluent: EA/PE=1/2) to obtain compound 218A as a yellow solid (200 mg, 84.5%).

LC-MS (ESI): m/z=374.2[M+H]⁺.

Step 2:

### 4-(5-((1S,2S)-2-fluorocyclopropane-1-carboxamido)benzo[d]oxazol-2-yl)-3-methoxypicolinic Acid (218B)

Compound 218A (200 mg, 0.54 mmol), tetrahydrofuran (2 mL), methanol (5 ml), distilled water (2 mL) and lithium hydroxide monohydrate (230 mg, 5.4 mmol) were successively added to a single-necked flask, and the mixture was stirred at room temperature for 3 h. The reaction was quenched by adding dilute hydrochloric acid and extracted with dichloromethane (50 mL×2). The combined organic phase was dried over anhydrous sodium sulfate and concentrated under reduced pressure to obtain a crude product of compound 218B (160 mg, 80%) from the residue. The crude product can be directly used in the next reaction.

LC-MS (ESI): m/z=372.0[M+H]⁺.

Step 3:

### (1S,2S)-2-fluoro-N-(2-(3-methoxy-2-(4-((R)-(5-methyl-2H-tetrazol-2-yl)(phenyl)methyl)piperidine-1-carbonyl)pyridin-4-yl)benzo[d]oxazol-5-yl)cyclopropane-1-carboxamide (Compound 218)

Compound 218B (160 mg, 0.43 mmol), DMF (10 mL), HATU (178 mg, 0.47 mmol), DIPEA (130 mg, 1 mmol) and compound 120B (120 mg, 0.47 mmol) were successively added to a single-necked flask, and the mixture was stirred at room temperature for 2 h. The reaction was quenched by adding saturated aqueous sodium bicarbonate solution (30 mL), extracted with dichloromethane (50 mL×1) and allowed to stand for phase separation. The aqueous phase was washed with dichloromethane (50 mL×2). The combined organic phase was dried over anhydrous sodium sulfate and concentrated under reduced pressure to obtain a crude product from the residue, and the crude product was separated by column chromatography (eluent: DCM/MeOH=40/1) to obtain compound 218 as a light yellow solid (0.12 g, 45.6%).

¹H NMR (400 MHz, CDCl₃) δ 8.50-8.48 (m, 1H), 8.10-8.07 (m, 2H), 7.71 (s, 1H), 7.60-7.48 (m, 4H), 7.42-7.29 (m, 3H), 5.53-5.50 (m, 1H), 4.92-4.75 (m, 2H), 4.07-4.04 (m, 3H), 3.37-3.33 (m, 1H), 3.13-3.02 (m, 1H), 2.90-2.79 (m, 2H), 2.55-2.47 (m, 3H), 1.92-1.84 (m, 2H), 1.45-1.26 (m, 5H).

LC-MS (ESI): m/z=611.2 [M+H]⁺.

### Example 219

### (1S,2S)-2-fluoro-N-(2-(3-fluoro-2-(4-((R/S)-(5-methyl-2H-tetrazol-2-yl)(phenyl)methyl)piperidine-1-carbonyl)pyridin-4-yl)benzo[d]oxazol-5-yl)cyclopropanecarboxamide

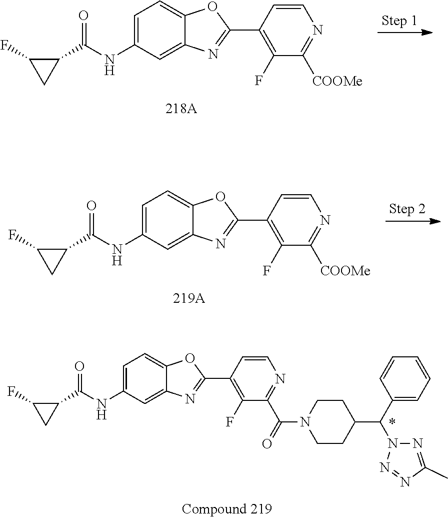 [SMILES: COC(=O)c1nccc(-c2nc3cc(NC(=O)[C@@H]4C[C@@H]4F)ccc3o2)c1F ; COC(=O)c1nccc(-c2nc3cc(NC(=O)[C@@H]4C[C@@H]4F)ccc3o2)c1F ; Cc1nnn(C(c2ccccc2)C2CCN(C(=O)c3nccc(-c4nc5cc(NC(=O)[C@@H]6C[C@@H]6F)ccc5o4)c3F)CC2)n1]

Step 1:

### 3-fluoro-4-(5-((1S,2S)-2-fluorocyclopropanecarboxamido)benzo[d]oxazol-2-yl)picolinic Acid (219A)

Compound 218A (100 mg, 0.27 mmol) was added to toluene (10 mL) at room temperature, and then tributyltin oxide (320 mg, 0.54 mmol) was added dropwise. After the dropwise addition was completed, the mixture was warmed to 100° C. and reacted overnight. The reaction solution was cooled to room temperature, then acidified to pH=4 with dilute hydrochloric acid (1.0 mol/L), filtered by suction and washed with water (5 mL×3). The filter cake was dried to obtain a brownish yellow crude product (200 mg), which was directly used in the next reaction without purification.

LC-MS (ESI): m/z=360.1 [M+H]⁺.

Step 2:

### (1S,2S)-2-fluoro-N-(2-(3-fluoro-2-(4-((R/S)-(5-methyl-2H-tetrazol-2-yl)(phenyl)methyl)piperidine-1-carbonyl)pyridin-4-yl)benzo[d]oxazol-5-yl)cyclopropanecarboxamide (Compound 219)

Compounds 219A and 120B were used as raw materials to obtain compound 219 according to the synthetic method of compound 85.

LC-MS (ESI): m/z=599.2 [M+H]⁺.

¹H NMR (400 MHz, CDCl₃) δ 8.54 (s, 1H), 8.16-8.06 (m, 2H), 7.96 (s, 1H), 7.55-7.49 (m, 4H), 7.42-7.30 (m, 3H), 5.53-5.51 (m, 1H), 4.89-4.73 (m, 2H), 3.49-3.47 (m, 1H), 3.17-3.10 (m, 1H), 2.89-2.81 (m, 2H), 2.55-2.48 (m, 3H), 1.92-1.87 (m, 2H), 1.62-1.60 (m, 2H), 1.47-1.36 (m, 2H), 1.22-1.26 (m, 1H).

### Test Part:

Test method: the cytopathic effect (CPE) method was used to test the antiviral activity of the compound of the present disclosure against influenza virus, and the MDCK cytotoxicity was also measured. Each compound was tested for 8 concentrations (double replicate wells). CCK-8 reagent was used to detect cell viability. MDCK cells were seeded at a certain density in a microplate and cultured overnight at 37° C. and 5% CO₂. The compounds and viruses were added the next day. Cell control (without compound treatment or virus infection) and virus infection control (cells infected with virus and without compound treatment) were provided. The final concentration of DMSO in the cell culture medium was 0.5%. The cells were cultured at 37° C. and 5% CO₂ for 5 days until the rate at which a cytopathic effect appears in virus control wells reached 80%-95%. CCK-8 reagent was used to detect cell viability, and the raw data was used to calculate the antiviral activity of the compounds. GraphPad Prism software was used to analyze dose response curves of the compounds and calculate EC₅₀ values. The results were shown in Table 1 and Table 2.

TABLE 1
Anti-proliferative activity results of compounds of the present
disclosure on IFVA/Mal/302/54 and IFVA/Weiss/43 viruses

| Example | EC₅₀(IFVA/Mal/302/54) μM | EC₅₀(IFVA/Weiss/43) μM |
|---|---|---|
| 1 | 0.573 | 0.480 |
| 2 | 0.026 | <0.005 |
| 3 | 1.167 | 0.780 |
| 4 | 0.020 | 0.017 |
| 5 | 0.024 | 0.016 |
| 6a | >10 | >7.873 |
| 6b | >10 | >10 |
| 7 | 0.841 | 0.520 |
| 8 | >10 | >10 |
| 9 | 0.214 | 0.126 |
| 10 | >10 | >10 |
| 11 | 0.26 | 0.173 |
| 12 | 0.055 | 0.022 |
| 13 | 7.832 | >10 |
| 14 | 1.104 | 0.383 |
| 15 | >10 | >10 |
| 16a | >10 | 7.159 |
| 16b | >10 | >10 |
| 17 | 0.022 | 0.009 |
| 18 | 0.177 | 0.422 |
| 19 | 0.019 | 0.023 |
| 20 | 0.919 | 0.545 |
| 21 | 0.436 | 0.163 |
| 22 | 0.033 | 0.025 |
| 23 | >10 | >10 |
| 24 | 0.032 | 0.012 |
| 25 | 0.068 | 0.060 |
| 26 | 0.020 | 0.003 |
| 27 | 0.127 | 0.033 |
| 28 | 0.027 | 0.017 |
| 29 | 0.022 | 0.004 |
| 30 | 0.027 | 0.012 |
| 31 | 0.007 | 0.002 |
| 32 | 0.003 | 0.026 |
| 33 | 0.031 | 0.016 |
| 34 | >10 | >10 |
| 35 | 0.019 | 0.012 |
| 36 | 0.010 | 0.004 |
| 37 | 0.022 | 0.017 |
| 38 | 0.018 | 0.020 |
| 39 | 0.011 | 0.004 |
| 40 | 0.024 | 0.016 |
| 42 | 0.010 | 0.020 |
| 43 | 0.010 | 0.057 |
| 44 | 0.065 | 0.110 |
| 45 | 0.012 | 0.015 |
| 47 | 0.081 | 0.035 |
| 49 | 0.180 | 0.112 |
| 50 | 0.120 | 0.242 |
| 51 | 0.034 | 0.084 |
| 56 | 0.033 | 0.017 |
| 59 | 0.058 | 0.056 |
| 60 | 0.084 | 0.054 |
| 61 | 0.078 | 0.028 |
| 67 | 0.021 | 0.012 |
| 70 | 0.032 | 0.032 |
| 71 | 0.052 | 0.031 |
| 72 | 0.004 | 0.004 |
| 73 | 0.006 | 0.002 |
| 75 | 0.006 | 0.002 |
| 79 | 0.013 | 0.007 |
| 82 | 0.011 | 0.007 |
| 85 | 0.004 | 0.004 |
| 88 | 0.002 | 0.002 |
| 90 | 0.018 | 0.028 |
| 95 | 0.010 | 0.017 |
| 96 | 0.007 | 0.012 |
| 97 | 0.006 | 0.007 |
| 101 | 0.006 | 0.004 |
| 103 | 0.017 | 0.007 |
| 104 | 0.016 | 0.012 |
| 105 | 0.009 | 0.007 |
| 107 | 0.006 | 0.002 |
| 108 | 0.006 | 0.002 |
| 109 | 0.011 | 0.006 |
| 110 | 0.003 | 0.003 |
| 111 | 0.002 | 0.0005 |
| 112 | 0.008 | 0.011 |
| 113 | 0.018 | 0.019 |
| 114 | 0.010 | 0.019 |
| 119 | 0.006 | 0.002 |
| 120 | 0.014 | 0.003 |
| 175 | 0.021 | 0.013 |
| 176 | 0.021 | 0.033 |
| 194 | 0.025 | 0.024 |
| 196 | 0.008 | 0.006 |

TABLE 2
Anti-proliferative activity results of compounds
of the present disclosure on IFV A/PR/8/34 virus

| Example | EC₅₀(IFV A/PR/8/34) μM |
|---|---|
| 36 | 0.017 |
| 42 | 0.020 |
| 45 | 0.027 |
| 67 | 0.017 |
| 72 | 0.004 |
| 73 | 0.003 |
| 75 | 0.002 |
| 85 | 0.004 |
| 93 | 0.016 |
| 101 | 0.027 |
| 103 | 0.026 |
| 104 | 0.059 |
| 105 | 0.011 |
| 107 | 0.009 |
| 108 | 0.023 |
| 109 | 0.012 |
| 111 | 0.002 |
| 112 | 0.036 |
| 113 | 0.020 |
| 114 | 0.025 |
| 115 | 0.064 |
| 119 | 0.022 |
| 120 | 0.012 |
| 122 | 0.019 |
| 122 | 0.019 |
| 125 | 0.012 |
| 126 | 0.001 |
| 127 | 0.004 |
| 129 | 0.012 |
| 130 | 0.029 |
| 131 | 0.012 |
| 132 | 0.004 |
| 133 | 0.033 |
| 134 | 0.004 |
| 135 | 0.023 |
| 136 | 0.008 |
| 137 | 0.011 |
| 138 | 0.004 |
| 139 | 0.004 |
| 140 | 0.004 |
| 141 | 0.016 |
| 142 | 0.023 |
| 143 | 0.013 |
| 144 | 0.007 |
| 146 | 0.026 |
| 147 | 0.014 |
| 148 | 0.033 |
| 150 | 0.008 |
| 151 | 0.038 |
| 152 | 0.005 |
| 153 | 0.007 |
| 154 | 0.005 |
| 155 | 0.001 |
| 156 | 0.007 |
| 158 | 0.003 |
| 160 | 0.018 |
| 161 | 0.020 |
| 162 | 0.003 |
| 163 | 0.008 |
| 164 | 0.013 |
| 165 | 0.010 |
| 166 | 0.006 |
| 167 | 0.005 |
| 168 | 0.002 |
| 170 | 0.007 |
| 171 | 0.005 |
| 172 | 0.013 |
| 174 | 0.015 |
| 178 | 0.007 |
| 182 | 0.006 |
| 183 | 0.018 |
| 184 | 0.014 |
| 185 | 0.024 |
| 186 | 0.002 |
| 187 | 0.006 |
| 189 | 0.011 |
| 190 | 0.011 |
| 197 | 0.0009 |
| 199 | 0.0018 |
| 200 | 0.0021 |
| 201 | 0.0036 |
| 202 | 0.0026 |
| 203 | 0.019 |
| 205 | 0.0055 |
| 206 | 0.0045 |
| 207 | 0.0013 |
| 208 | 0.0069 |
| 210 | 0.0008 |
| 211 | 0.0003 |
| 212 | 0.0022 |
| 214 | 0.0019 |
| 217 | 0.0033 |
| 219 | 0.0019 |

### CONCLUSION

The compounds of the present disclosure exhibit good anti-proliferative activity against IFVA/Mal/302/54, IFVA/Weiss/43 and IFV A/PR/8/34 viruses.

1. A compound represented by general formula (I-a), an isomer thereof or a pharmaceutically acceptable salt thereof, 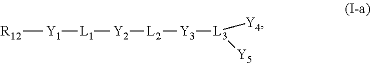
wherein:
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally further substituted with 0-5 R^{y1};
Y₂ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y2};
Y₃ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y3};
Y₄ is selected from C₃₋₁₂ carbocyclyl, C₅₋₁₅ aryl, 5-15 membered heteroaryl, 3-12 membered heterocyclyl, C₂₋₈ alkenyl and C₂₋₈ alkynyl, wherein the carbocyclyl, aryl, heteroaryl, heterocyclyl, alkenyl and alkynyl are optionally substituted with 0-5 R^{y4};
Y₅ is selected from C₃₋₁₂ carbocyclyl, C₅₋₁₅ aryl, 5-15 membered heteroaryl, 3-12 membered heterocyclyl, —C(O)NHOCH₃, —C(O)NHCN, —P(O)(OCH₃)₂ and —C(O)OCH₂CH₃, wherein the carbocyclyl, aryl, heteroaryl and heterocyclyl are optionally substituted with 0-5 R⁵;
R^{y1}, R^{y2}, R^{y3}, R^{y4} and R^{y5} are each independently selected from deuterium, halogen, ═O, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ hydroxylalkyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl, 5-10 membered heteroaryl or acetoxy; or
R^{y3} and the atom on Y₃ ring form C₃₋₈ cycloalkyl; or
R^{y1} and R^{y2} together with L₁ form 4-8 membered heterocyclyl, C4-s carbocyclyl or 5-8 membered heteroaryl;
R₁₂ is selected from H, cyano, C₁₋₈ alkoxy, C₃₋₈ cycloalkyl, 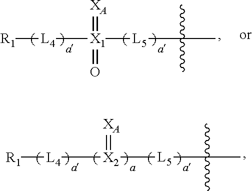
3-8 membered heterocyclyl, C₅₋₁₀ aryl or 5-10 membered heteroaryl, wherein the cycloalkyl, heterocyclyl, aryl or heteroaryl is optionally further substituted with 0-5 of the following groups: halogen, ═O, acetamido, hydroxyl, cyano, sulfhydryl, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
X₁ is selected from P or S;
X₂ is selected from C, PR₁ₐ or S;
L₄ and L₅ are each independently selected from: a bond, NR₂, CR₃R₄, O, or S;
X_{A} is selected from C, NR₂ₐ, O and S;
a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, C₁₋₈ alkyl, cyano, ═O, C₁₋₈ alkoxy or C₁₋₈ haloalkyl;
R₁ₐ, R₂, R₂ₐ, R₃ and R₄ are each independently selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: halogen, C₁₋₈ alkyl, C₁₋₈ alkoxy or C₁₋₈ haloalkyl;
L₁ is selected from a bond, —(CR₆)ₜ—NR₅—(CR₇)_{t′}—, C₂₋₈ alkynyl, C₂₋₈ alkenyl, 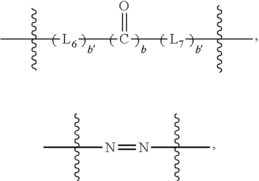
3-8 membered heterocyclyl, C₃₋₈ cycloalkyl, C₅₋₁₀ aryl or 5-10 membered heteroaryl, wherein the alkynyl, alkenyl, heterocyclyl, cycloalkyl, aryl or heteroaryl is optionally substituted with 0-5 groups selected from: hydroxyl, halogen, C₁₋₈ alkyl or C₁₋₈ haloalkyl;
t and t′ are selected from 0, 1, 2 and 3;
L₂ is selected from a bond, 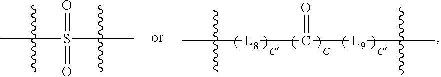
L₆, L₇, L₈ and L₉ are each independently selected from a bond, O, S, NRs or CR₆R₇;
b and c are each independently selected from 0 or 1;
b′ and c′ are each independently selected from 0, 1, 2, 3, 4, 5 or 6;
R₅, R₆ and R₇ are each independently selected from: H, halogen, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl or 3-8 membered heterocyclyl;
L₃ is selected from a bond, 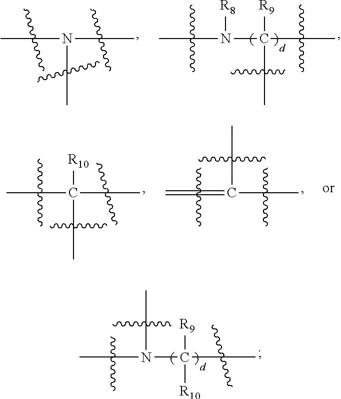
d is an integer selected from 1-6;
and R₈, R₉ and R₁₀ are each independently selected from H, halogen, sulfhydryl, cyano, nitro, C₁₋₈ alkyl, C₂₋₈ alkenyl, C₂₋₈ alkynyl, C₃₋₈ cycloalkyl, ═O, C₁₋₈ alkoxy, C₁₋₈ haloalkyl or 3-8 membered heterocyclyl;
provided that:
L₁, L₂ and L₃ are not simultaneously a bond;
when Y₁ is selected from 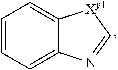
X^{y1} is O or S, L₁ is a bond, L₂ is carbonyl, L₃ is 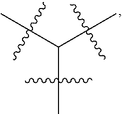 [SMILES: *C(*)*]
Y₂ is selected from 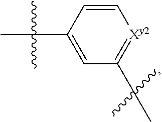
X^{y2} is C or N, Y₃ is selected from 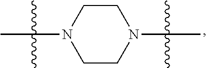 [SMILES: *N1CCN(*)CC1]
Y₄ is selected from a benzene ring, Y₁ is substituted with 0 R^{y1}, and R₁₂ is selected from acetamido, halogen, methyl, methoxy, trifluoromethyl, trifluoromethyloxy, hydroxyl, methanesulfonylamido, methylamino or N,N-dimethylamino, Y₅ is not selected from the following groups: 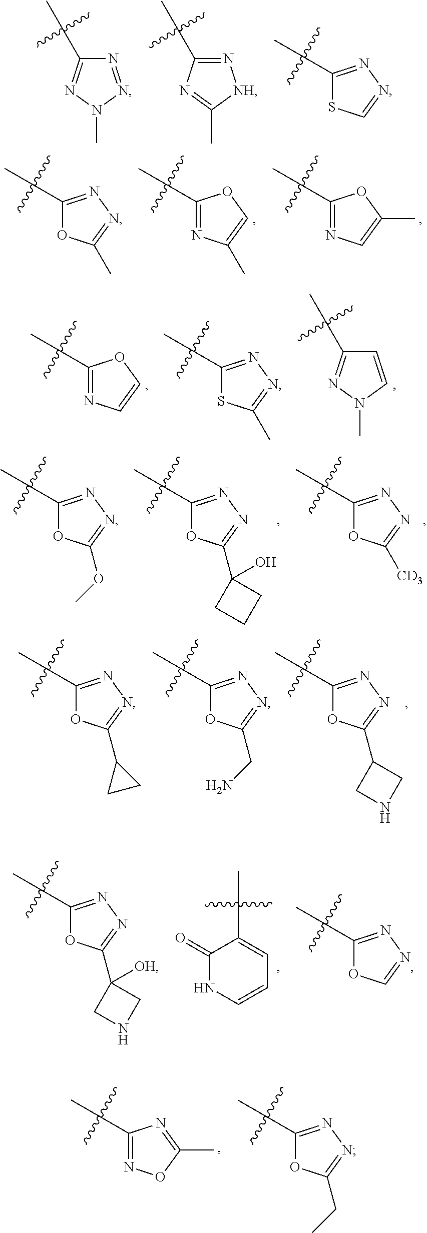
when R₁₂—Y₁ is selected from 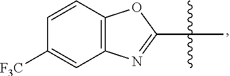 [SMILES: *c1nc2cc(C(F)(F)F)ccc2o1]
L₁ is a bond, L₂ is carbonyl, L₃ is 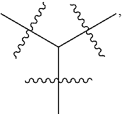 [SMILES: *C(*)*]
Y₃ is selected from 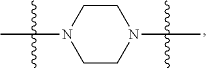 [SMILES: *N1CCN(*)CC1]
Y₄ is selected from a benzene ring and Y₅ is selected from 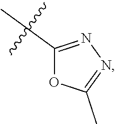 [SMILES: *c1nnc(C)o1]
Y₂ is not selected from the following groups: 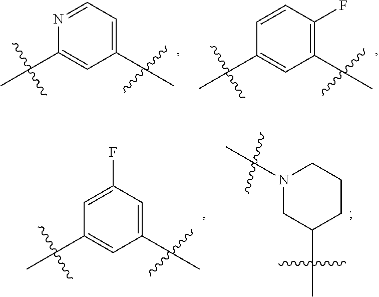 [SMILES: *c1ccnc(*)c1 ; *c1ccc(F)c(*)c1 ; *c1cc(*)cc(F)c1 ; *C1CCCN(*)C1]
when Y₅ is selected from acetamido, L₃ is a bond;
and when Y₅ is —C(O)OCH₂CH₃, it does not have the following structure:

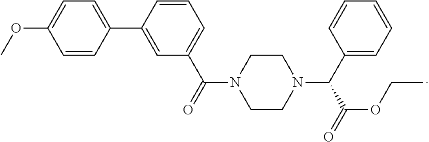 [SMILES: CCOC(=O)[C@@H](c1ccccc1)N1CCN(C(=O)c2cccc(-c3ccc(OC)cc3)c2)CC1]

2. The compound of formula (I-a), the isomer thereof or the pharmaceutically acceptable salt thereof according to claim 1, having a structure of formula (I-1), 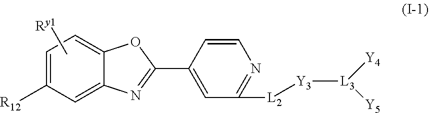
wherein Y₃ is selected from 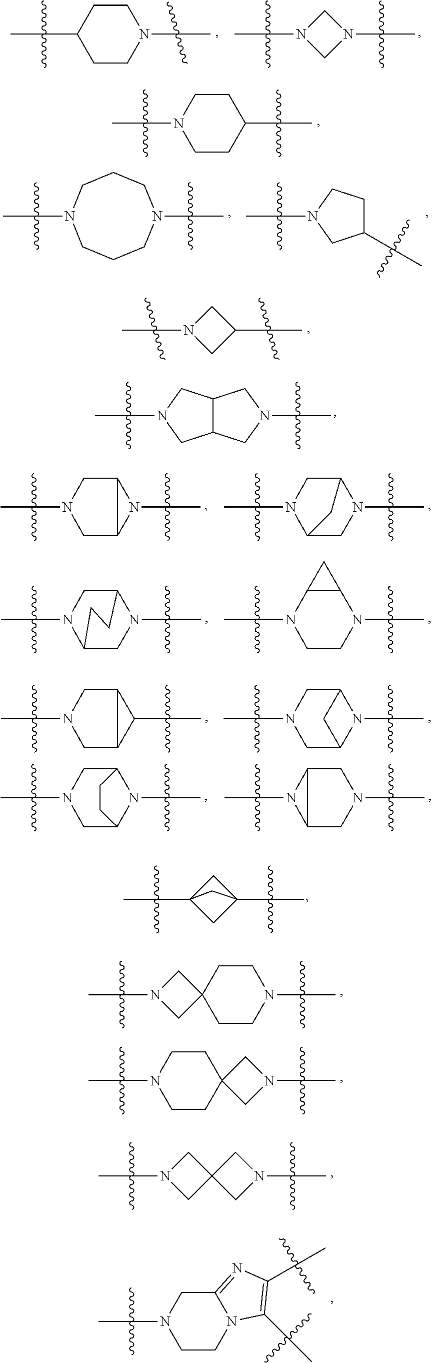 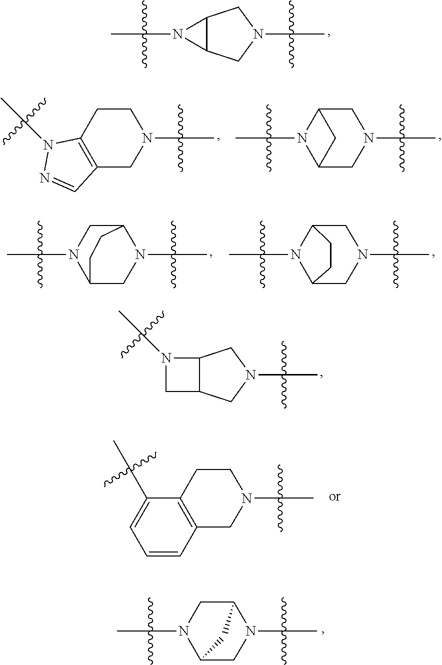
wherein the above-mentioned groups are optionally further substituted with 0 to 3 of the following groups: halogen, hydroxyl, cyano, C₁₋₈ alkyl or C₁₋₈ haloalkyl;
Y₄ is selected from phenyl;
Y₅ is selected from 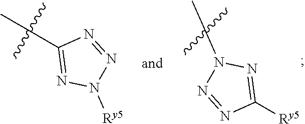
R^{y5} is selected from methyl or fluoroethyl;
L₂ is selected from carbonyl;
L₃ is selected from 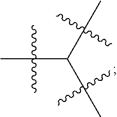 [SMILES: *C(*)*]
R₁₂ is selected from acetamido, cyclopropylacetamido, fluorocyclopropylacetamido or 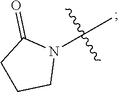 [SMILES: *N1CCCC1=O]
and R^{y1} is selected from H.

3. The compound of formula (I-a), the isomer thereof or the pharmaceutically acceptable salt thereof as shown in claim 1, wherein
Y₄ is selected from phenyl optionally substituted with 0-5 R^{y4};
and Y₅ is selected from tetrazolyl and 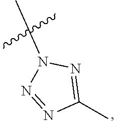 [SMILES: *n1nnc(C)n1]
wherein the tetrazolyl is further substituted with monofluoromethyl, difluoromethyl, fluoroethyl, hydroxymethyl, hydroxyethyl and methoxymethyl.

4. The compound of formula (I-a), the isomer thereof or the pharmaceutically acceptable salt thereof as shown in claim 3, having a structure of formula (I-2), 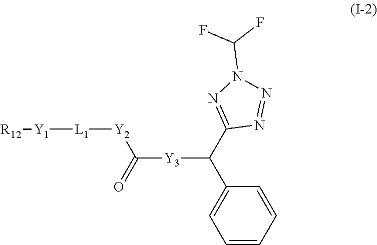
wherein,
Y₁ is selected from C₃₋₁₂ carbocyclyl, C₅₋₁₅ aryl, 5-15 membered heteroaryl and 3-12 membered heterocyclyl, wherein the carbocyclyl, aryl, heteroaryl and heterocyclyl are optionally substituted with 0-5 R^{y1};
R₁₂ is selected from cyano, 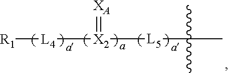
3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R^{y1} is selected from halogen, cyano and C₁₋₈ haloalkyl; or
R^{y1} and R^{y2} together with L₁ form 4-8 membered heterocyclyl, C4-s carbocyclyl or 5-8 membered heteroaryl;
X₂ is selected from C;
L₄ and L₅ are each independently selected from: a bond, NR₂, CR₃R₄, O, or S;
X_{A} is selected from O and S;
a is 0 or 1;
a′ is selected from 0, 1, 2, 3, 4, 5 or 6;
R₁ is selected from: H, halogen, ═O, sulfhydryl, cyano, nitro, C₁₋₈ alkyl, C₂₋₈ alkenyl, C₂₋₈ alkynyl, C₃₋₈ cycloalkyl, C₁₋₈ alkoxy, C₁₋₈ haloalkyl, 3-8 membered heterocyclyl, C₅₋₁₀ aryl or 5-10 membered heteroaryl, wherein the alkyl, alkenyl, alkynyl, cycloalkyl, alkoxy, haloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl; and R₂, R₃ and R₄ are each independently selected from: H, halogen, C₁₋₈ alkyl or C₁₋₈ haloalkyl.

5. The compound of formula (I-2), the isomer thereof or the pharmaceutically acceptable salt thereof according to claim 4, wherein
Y₃ is selected from 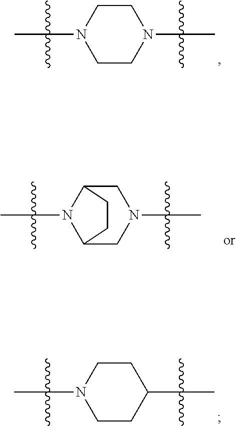 [SMILES: *N1CCN(*)CC1 ; *N1CC2CCC(C1)N2* ; *C1CCN(*)CC1]
and/or
Y₂ is selected from 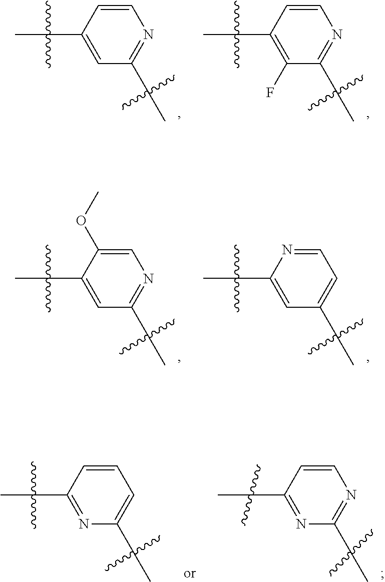
and/or
L₁ is selected from a bond,

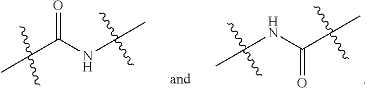 [SMILES: *NC(*)=O ; *NC(*)=O]

6. The compound of formula (I-2), the isomer thereof or the pharmaceutically acceptable salt thereof according to claim 5, wherein
Y₃ is selected from 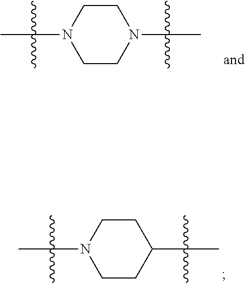 [SMILES: *N1CCN(*)CC1 ; *C1CCN(*)CC1]
and/or
Y₂ is selected from 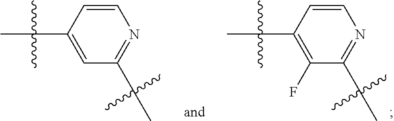 [SMILES: *c1ccnc(*)c1 ; *c1ccnc(*)c1F]
and/or
L₁ is selected from a bond.

7. The compound of formula (I-2), the isomer thereof or the pharmaceutically acceptable salt thereof according to claim 4, wherein
Y₁ is selected from phenyl, 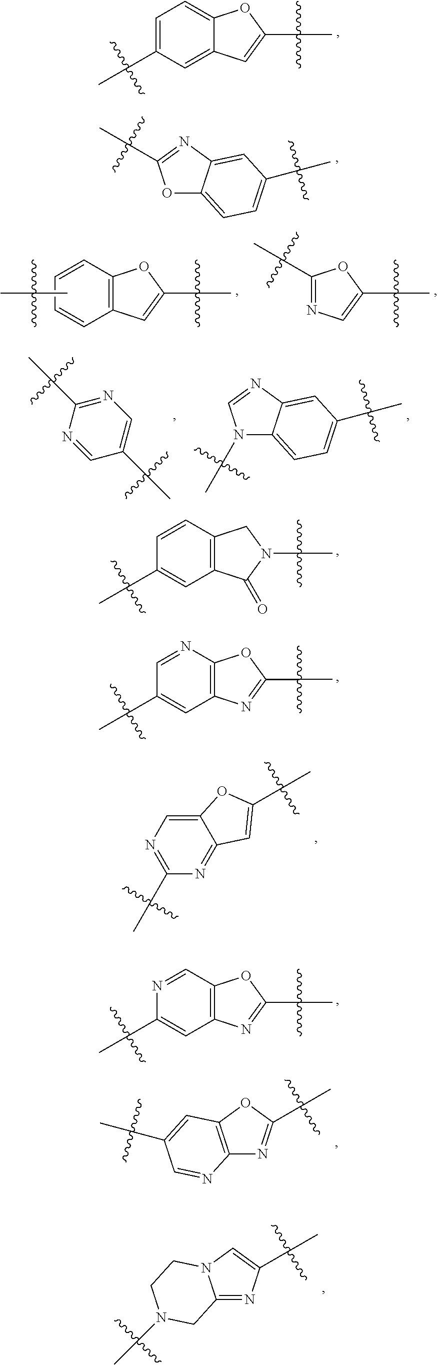 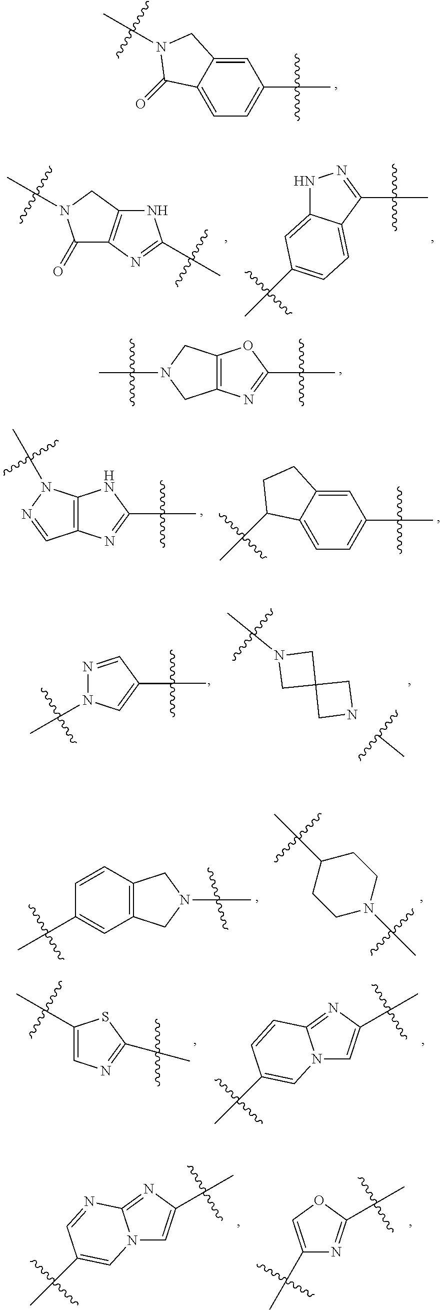 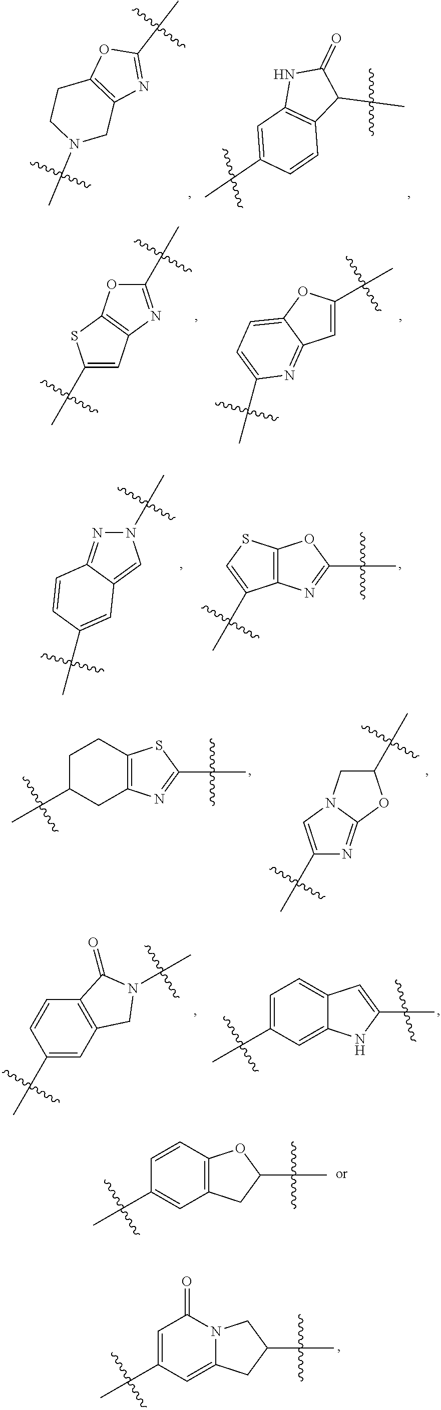
wherein the above-mentioned groups are optionally substituted with 0-3 R^{y1}.

8. The compound of formula (I-2), the isomer thereof or the pharmaceutically acceptable salt thereof according to claim 7, wherein
Y₁ is selected from 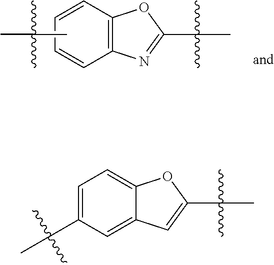
and optionally substituted with 0-3 R^{y1};
and R^{y1} is selected from F, methyl, difluoromethyl or trifluoromethyl.

9. The compound of formula (I-2), the isomer thereof or the pharmaceutically acceptable salt thereof according to claim 7, wherein
Y₁ is selected from 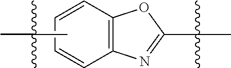
and optionally substituted with 0-3 R^{y1};
and R^{y1} is selected from F, methyl, difluoromethyl or trifluoromethyl.

10. The compound of formula (I-2), the isomer thereof or the pharmaceutically acceptable salt thereof according to claim 3, wherein
R₁₂ is selected from 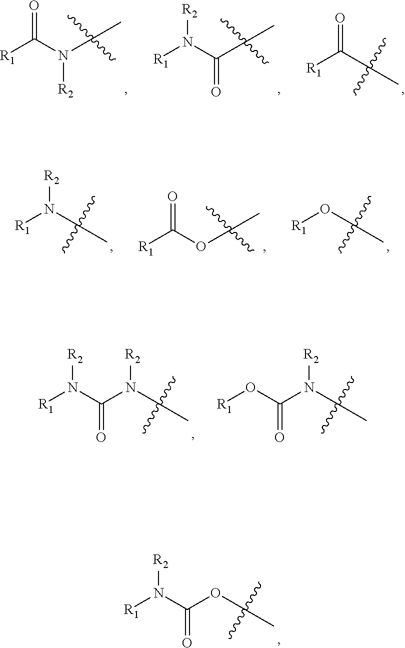
3-8 membered heterocyclyl, C₅₋₁₀ aryl or 5-10 membered heteroaryl, wherein the heterocyclyl, aryl, and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
and each R₂ is independently selected from: H or C₁₋₈ alkyl.

11. The compound of formula (I-2), the isomer thereof or the pharmaceutically acceptable salt thereof according to claim 10, wherein
R₁₂ is selected from 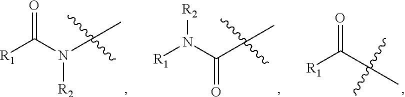
3-8 membered heterocyclyl or 5-6 membered heteroaryl, wherein the heterocyclyl and heteroaryl are optionally substituted with 0-3 of the following groups: halogen, ═O, acetamido, hydroxyl, sulfhydryl, cyano, nitro, C₁₋₄ alkyl, C₁₋₄ alkoxy, C₁₋₄ haloalkyl, C₂₋₄ alkenyl, C₂₋₄ alkynyl, C₃₋₆ cycloalkyl, 3-6 membered heterocyclyl or 5-6 membered heteroaryl;
R₁ is selected from: C₁₋₄ alkyl, C₃₋₆ cycloalkyl, 3-6 membered heterocyclyl or 5-6 membered heteroaryl, wherein the alkyl, cycloalkyl, heterocyclyl or heteroaryl is optionally further substituted with 0-3 groups selected from: deuterium, hydroxyl, halogen, cyano, ═O, C₁₋₄ alkyl, C₁₋₄ alkoxy or C₁₋₄ haloalkyl;
and each R₂ is independently selected from: H or C₁₋₂ alkyl.

12. The compound of formula (I-2), the isomer thereof or the pharmaceutically acceptable salt thereof according to claim 10, wherein
R₁₂ is selected from acetyl, pyridyl, thiazolyl, pyrazinyl, acetamido,

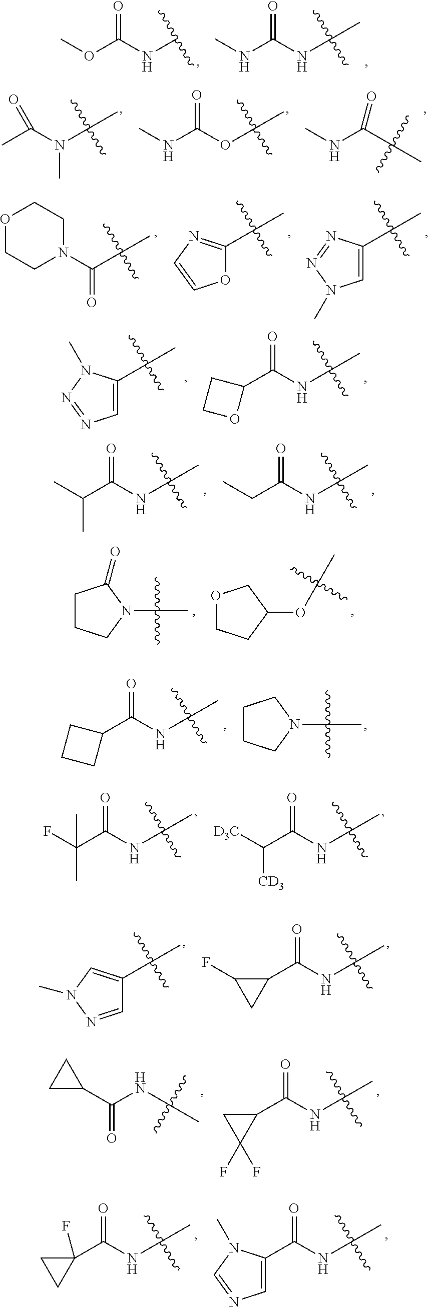

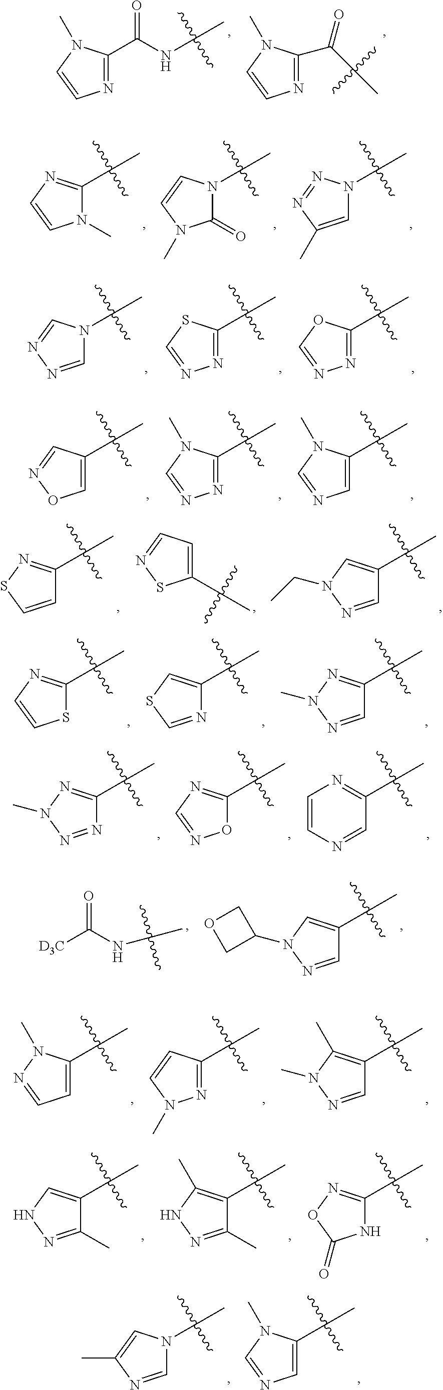

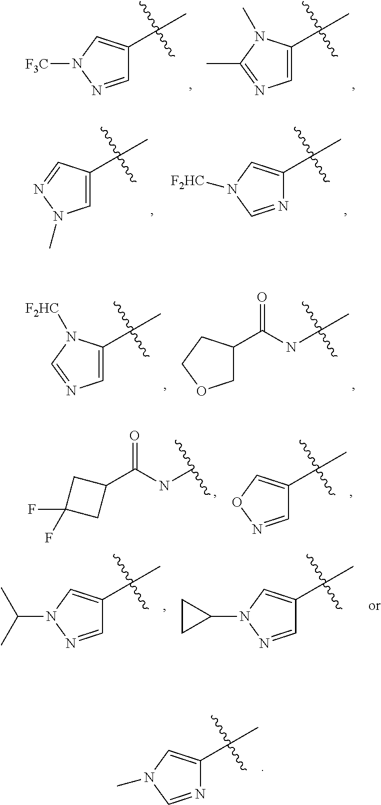

13. The compound of formula (I-2), the isomer thereof or the pharmaceutically acceptable salt thereof according to claim 3, wherein
R₁₂ is selected from

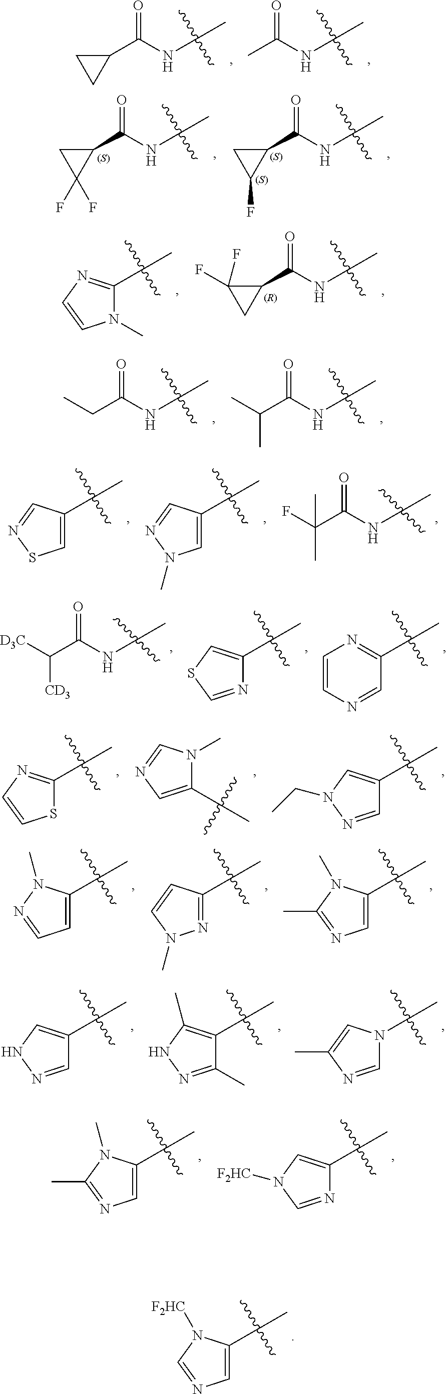

14. The compound of formula (I-a), the isomer thereof or the pharmaceutically acceptable salt thereof according to claim 1, wherein
R¹² is selected from 3-8 membered heterocyclyl, 5-10 membered heteroaryl or 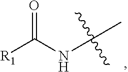
wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy, C₃₋₈ cycloalkyl, 3-8 membered heterocyclyl, or C₁₋₈ haloalkyl;
R₁ is selected from C₃₋₈ cycloalkyl, wherein the cycloalkyl is optionally further substituted with 0-3 halogen groups;
and R^{y1} is selected from halogen, cyano and C₁₋₈ haloalkyl.

15. The compound of formula (I-a), the isomer thereof or the pharmaceutically acceptable salt thereof according to claim 14, having a structure of formula (I-3) or formula (I-3-1),
wherein 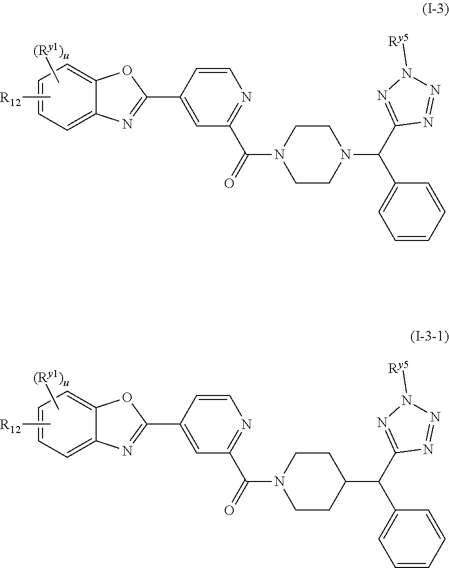
R₁₂ is selected from 3-8 membered heterocyclyl, 5-10 membered heteroaryl and 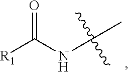
wherein the heterocyclyl and heteroaryl are optionally substituted with 0 to 3 of the following groups: halogen, ═O, acetamido, hydroxyl, sulfhydryl, cyano, nitro, C₁₋₈ alkyl, C₁₋₈ alkoxy, C₃₋₈ cycloalkyl or C₁₋₈ haloalkyl;
R^{y1} is selected from F, methyl, difluoromethyl or trifluoromethyl;
R^{y5} is selected from C₁₋₈ alkyl, C₁₋₈ haloalkyl, C₁₋₈ alkyl-C₁₋₈ alkoxy, C₁₋₈ hydroxylalkyl or C₃₋₈ cycloalkyl;
and u is selected from 0, 1 or 2.

16. The compound of formula (I-a), the isomer thereof or the pharmaceutically acceptable salt thereof according to claim 15, wherein
R₁₂ is selected from 5-6 membered heteroaryl and 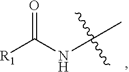
wherein the heteroaryl is optionally substituted with 0 to 3 of the following groups: halogen, ═O, acetamido, hydroxyl, C₁₋₄ alkyl, C₁₋₄ alkoxy, C₃₋₆ cycloalkyl or C₁₋₄ haloalkyl;
R^{y5} is selected from methyl, monofluoromethyl, difluoromethyl, monofluoroethyl, trifluoromethyl, hydroxymethyl, hydroxyethyl and methoxymethyl;
and u is selected from 0.

17. The compound, the isomer thereof or the pharmaceutically acceptable salt thereof according to claim 14, wherein R₁₂ is selected from

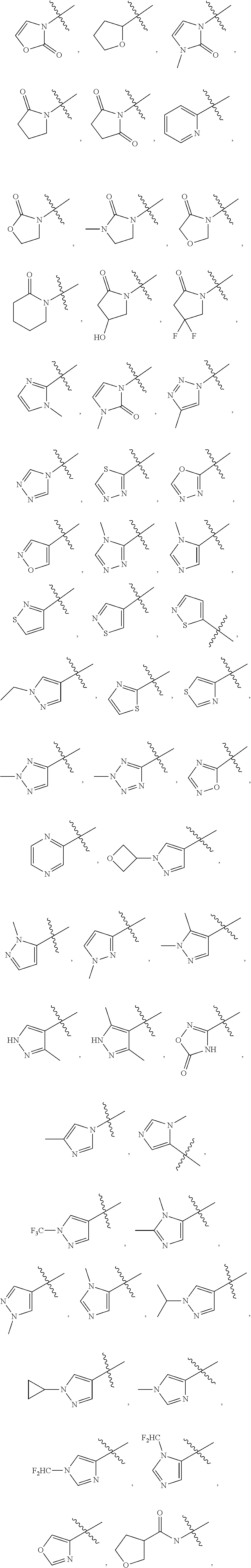

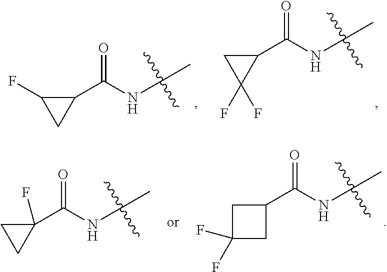 [SMILES: *NC(=O)C1CC1F ; *NC(=O)C1CC1(F)F ; *NC(=O)C1(F)CC1 ; *NC(=O)C1CC(F)(F)C1]

18. The compound of formula (I-a), the isomer thereof or the pharmaceutically acceptable salt thereof according to claim 1, having a structure of formula (I-9) or (I-9-1), 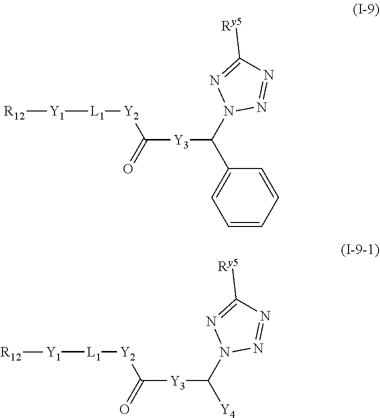
wherein each R^{y5} is independently selected from deuterium, halogen, hydroxyl, cyano, nitro, C₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl or 5-10 membered heteroaryl, wherein the alkyl, alkoxy, alkenyl, alkynyl, cycloalkyl, heterocyclyl, aryl and heteroaryl are optionally further substituted with 0-5 groups selected from: deuterium, halogen, ═O, acetamido, hydroxyl, sulfhydryl, cyano, nitro, silyl, C₁₋₈ hydroxylalkyl, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl, 5-10 membered heteroaryl and acetoxy;
Y₄ is selected from 5-15 membered heteroaryl or phenyl, wherein the phenyl is further substituted with 1-5 R^{y4}, and the heteroaryl is optionally substituted with 1-5 R^{y4};
R¹² is selected from cyano, 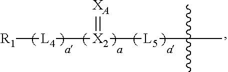
3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
and L₁ is selected from a bond.

19. The compound of formula (I-9) or (I-9-1), the isomer thereof or the pharmaceutically acceptable salt thereof according to claim 18, wherein
R^{y5} is selected from C₁₋₃ alkyl and C₃₋₈ cycloalkyl, wherein the alkyl is optionally further substituted with 0-5 groups selected from: deuterium, hydroxyl, acetoxy, halogen, C₁₋₃ alkoxy, amino and C₁₋₃ alkylamino;
and Y₄ is selected from 5 membered heteroaryl, or phenyl which is further substituted with 1-5 deuterium and halogen.

20. The compound of formula (I-9) or (I-9-1), the isomer thereof or the pharmaceutically acceptable salt thereof according to claim 19, wherein
Y₁ is selected from 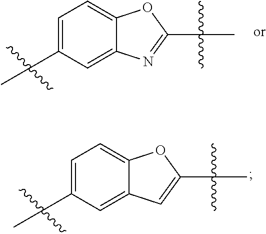 [SMILES: *c1ccc2oc(*)nc2c1 ; *c1ccc2oc(*)cc2c1]
Y₂ is selected from 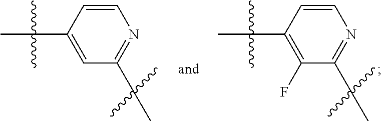 [SMILES: *c1ccnc(*)c1 ; *c1ccnc(*)c1F]
or selected from 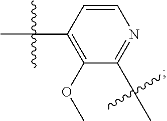 [SMILES: *c1ccnc(*)c1OC]
Y₃ is selected from 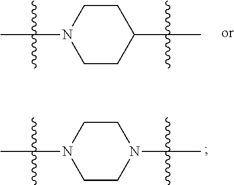 [SMILES: *C1CCN(*)CC1 ; *N1CCN(*)CC1]
and R^{y5} is selected from methyl, difluoromethyl, fluoroethyl, hydroxymethyl, hydroxyethyl, cyclopropyl, cyclobutyl, aminomethyl, —CH₂NHCH₃, —CH₂OC(O)CH₃ or —CD₃.

21. The compound of formula (I-9) or (I-9-1), the isomer thereof or the pharmaceutically acceptable salt thereof according to claim 18, wherein
Y₁ is selected from 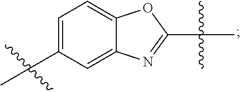 [SMILES: *c1ccc2oc(*)nc2c1]
Y₂ is selected from 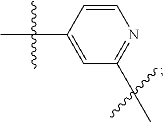 [SMILES: *c1ccnc(*)c1]
Y₃ is selected from 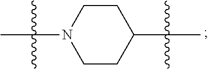 [SMILES: *C1CCN(*)CC1]
and R^{y5} is selected from methyl, difluoromethyl and hydroxymethyl.

22. The compound of formula (I-9) or (I-9-1), the isomer thereof or the pharmaceutically acceptable salt thereof according to claim 18, wherein
R₁₂ is selected from 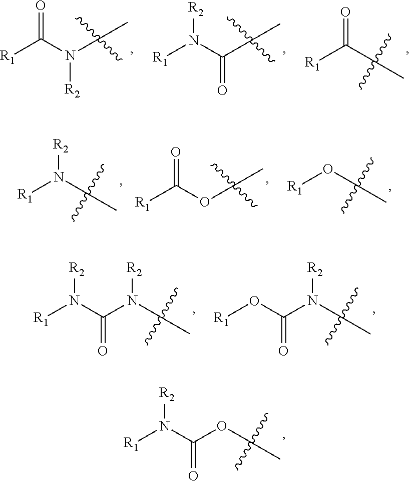
3-8 membered heterocyclyl, C₅₋₁₀ aryl or 5-10 membered heteroaryl, wherein the heterocyclyl, aryl and heteroaryl are optionally substituted with 0-5 of the following groups: halogen, ═O, acetamido, hydroxyl, sulfhydryl, cyano, nitro, C₁₋₈ alkyl, C₁₋₈ alkoxy, C₁₋₈ haloalkyl, C₂₋₈ alkenyl, C₂₋₈ alkynyl, C₃₋₈ cycloalkyl, 3-8 membered heterocyclyl, C₅₋₁₀ aryl or 5-10 membered heteroaryl;
R₁ is selected from: C₁₋₈ alkyl, C₃₋₈ cycloalkyl, 3-8 membered heterocyclyl, C₅₋₁₀ aryl or 5-10 membered heteroaryl, wherein the alkyl, cycloalkyl, heterocyclyl, aryl or heteroaryl is optionally further substituted with 0-5 groups selected from: deuterium, hydroxyl, halogen, cyano, ═O, C₁₋₈ alkyl, C₁₋₈ alkoxy or C₁₋₈ haloalkyl;
and each R₂ is independently selected from: H or C₁₋₈ alkyl.

23. The compound of formula (I-9) or (I-9-1), the isomer thereof or the pharmaceutically acceptable salt thereof according to claim 18, wherein
R₁₂ is selected from 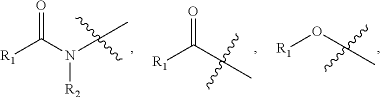
5 membered heteroaryl and 6 membered heteroaryl, wherein the heteroaryl is optionally substituted with 0-3 of the following groups: halogen, C₁₋₄ alkyl, C₁₋₄ alkoxy and C₁₋₄ haloalkyl;
R₁ is selected from three membered cycloalkyl, four membered cycloalkyl, five membered cycloalkyl and 5 membered heterocycloalkyl, wherein the cycloalkyl and heterocycloalkyl are optionally further substituted with 0-2 groups selected from: deuterium, hydroxyl, halogen, cyano, ═O, C₁₋₂ alkyl, C₁₋₂ alkoxy or C₁₋₂ haloalkyl;
and each R₂ is independently selected from: H or C₁₋₂ alkyl.

24. The compound of formula (I-9) or (I-9-1), the isomer thereof or the pharmaceutically acceptable salt thereof according to claim 18, wherein
Y₁ is selected from 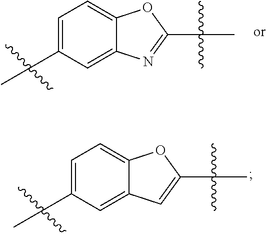 [SMILES: *c1ccc2oc(*)nc2c1 ; *c1ccc2oc(*)cc2c1]
Y₂ is selected from 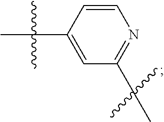 [SMILES: *c1ccnc(*)c1]
Y₃ is selected from 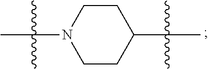 [SMILES: *C1CCN(*)CC1]
L₁ is selected from a bond;
R^{y5} is selected from methyl, difluoromethyl and hydroxymethyl;
R₁₂ is selected from 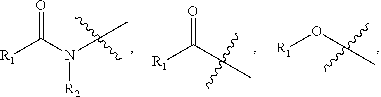
5 membered heteroaryl and 6 membered heteroaryl, wherein the heteroaryl is optionally substituted with 0-3 of the following groups: halogen, C₁₋₄ alkyl, C₁₋₄ alkoxy and C₁₋₄ haloalkyl;
R₁ is selected from 3 membered cycloalkyl, 4 membered cycloalkyl, 5 membered cycloalkyl, 5 membered heterocycloalkyl and 5 membered heteroaryl, wherein the cycloalkyl and heterocycloalkyl are optionally further substituted with 0-2 groups selected from: deuterium, hydroxyl, halogen, cyano, ═O, C₁₋₂ alkyl, C₁₋₂ alkoxy or C₁₋₂ haloalkyl;
and each R₂ is independently selected from: H or C₁₋₂ alkyl.

25. The compound of formula (I-9) or (I-9-1), the isomer thereof or the pharmaceutically acceptable salt thereof according to claim 18, wherein
R₁₂ is selected from acetyl, pyridyl, thiazolyl, pyrazinyl, acetamido,

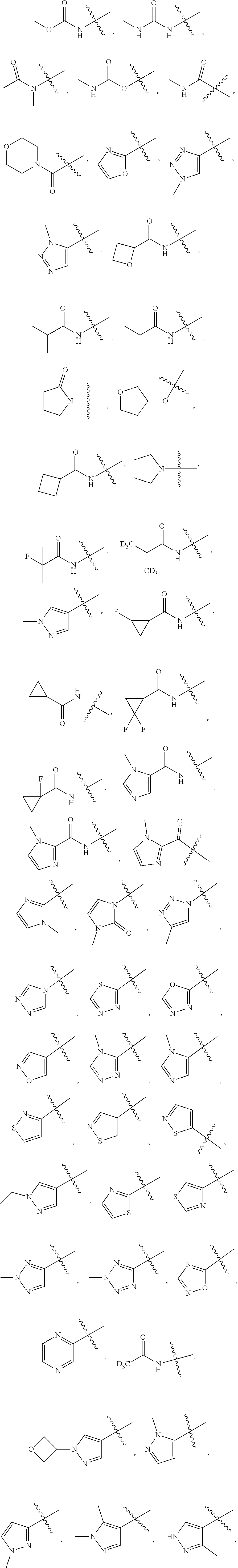

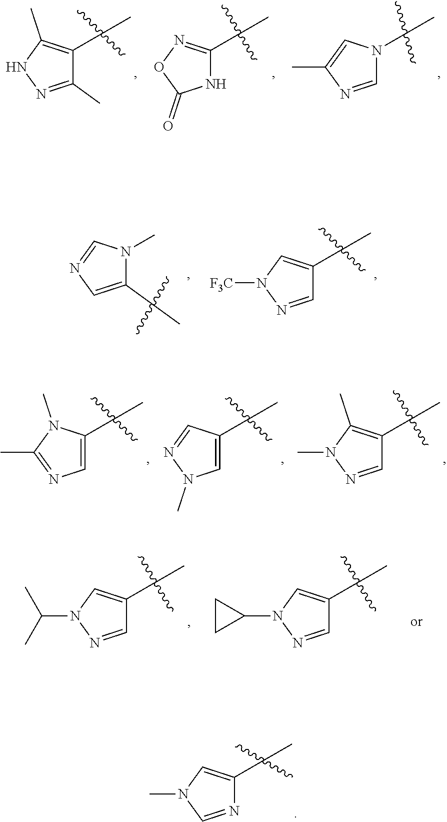

26. The compound, the isomer thereof or the pharmaceutically acceptable salt thereof according to claim 1, wherein R₁₂ is selected from

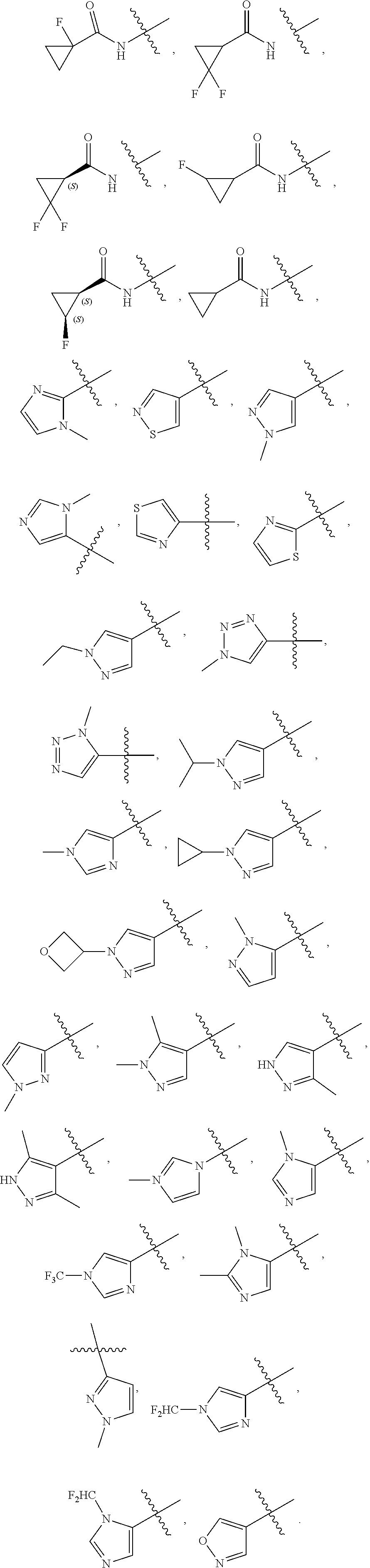

27. The compound, the isomer thereof or the pharmaceutically acceptable salt thereof according to claim 1, wherein the compound is selected from:

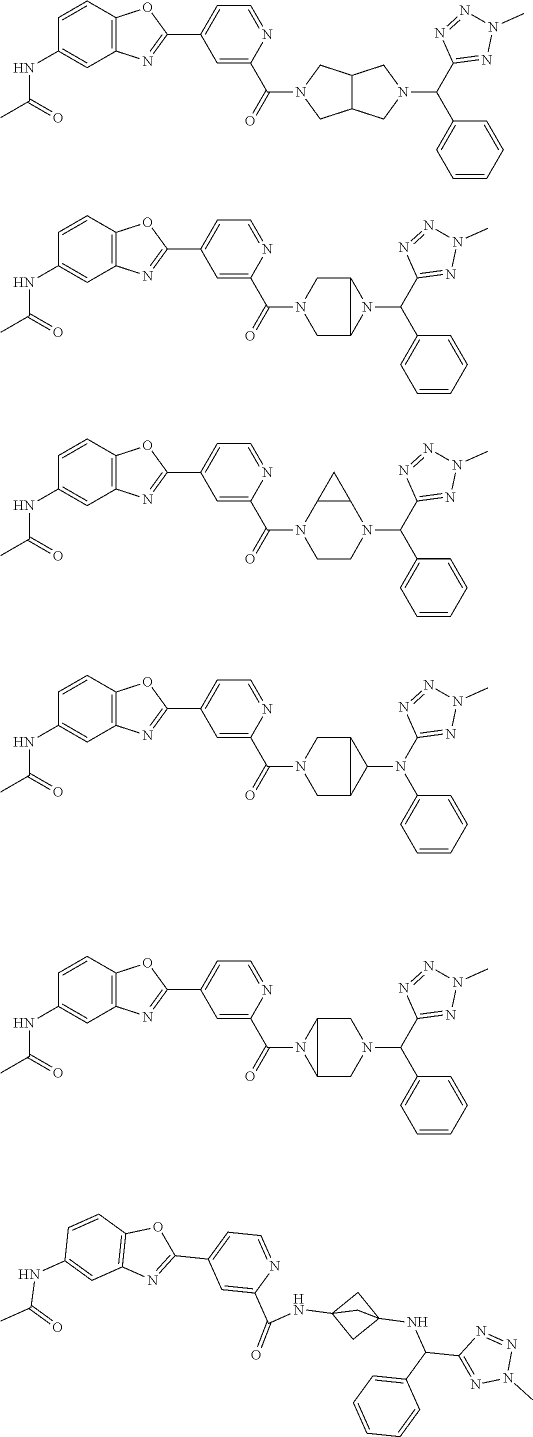

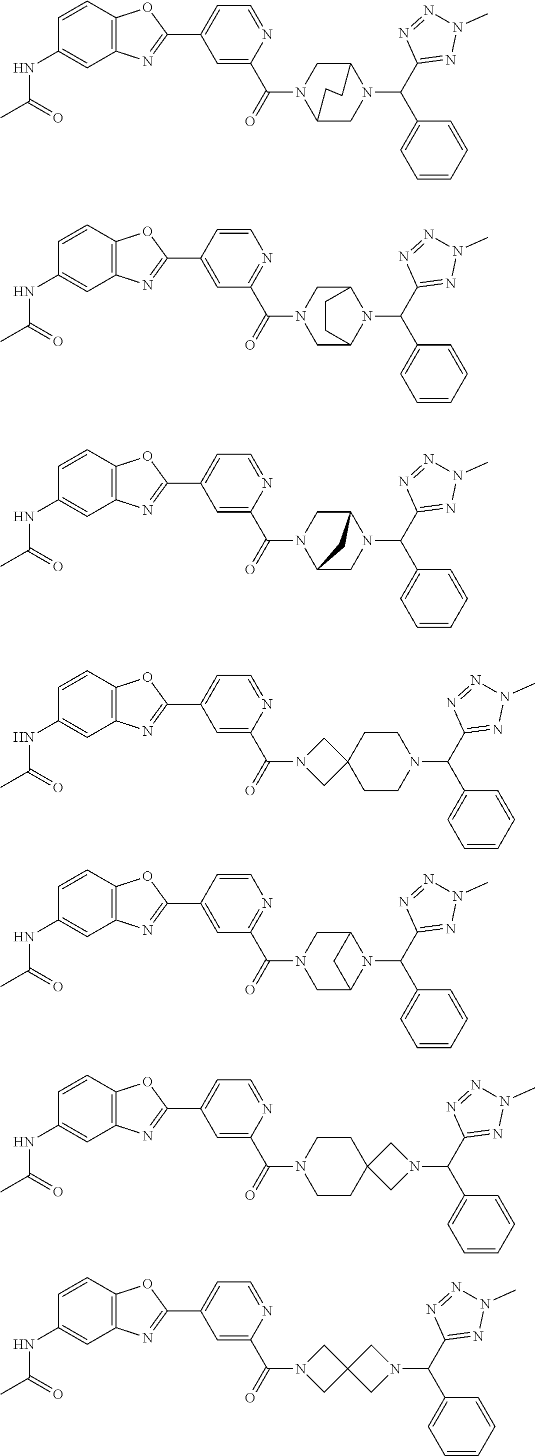

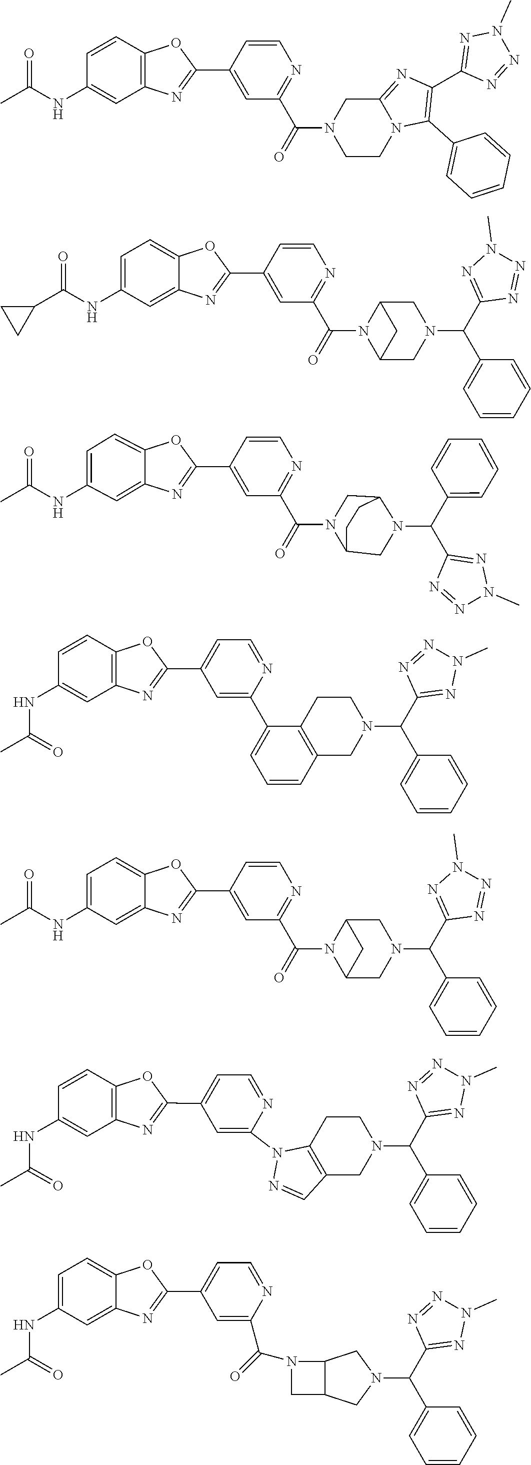

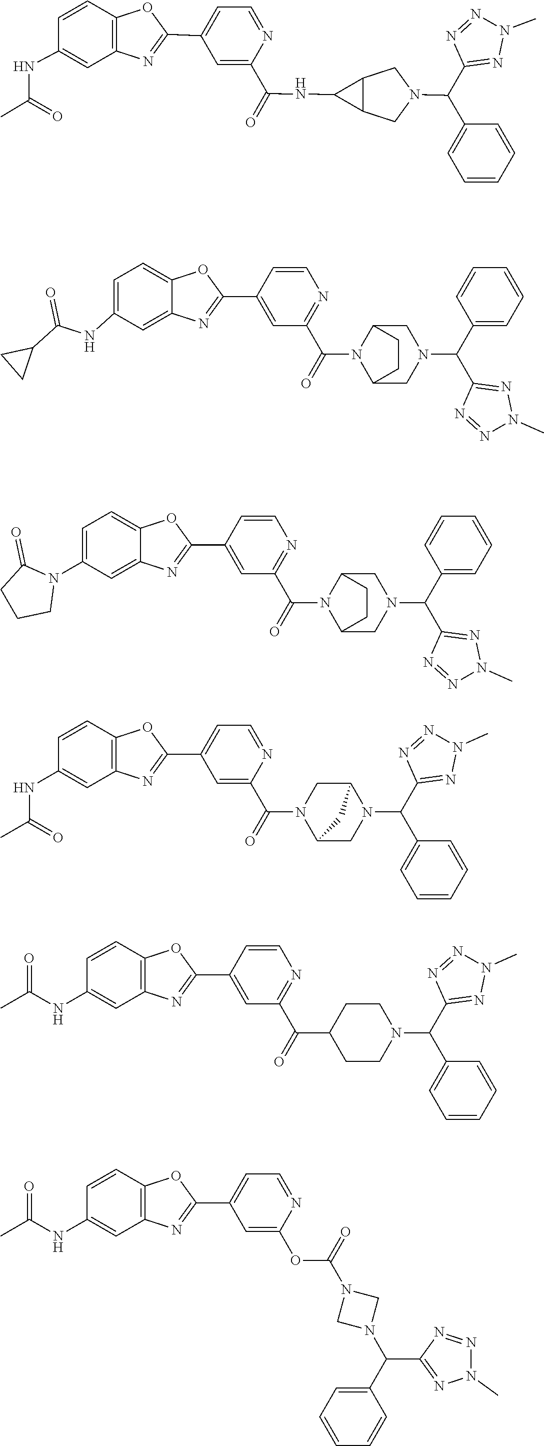

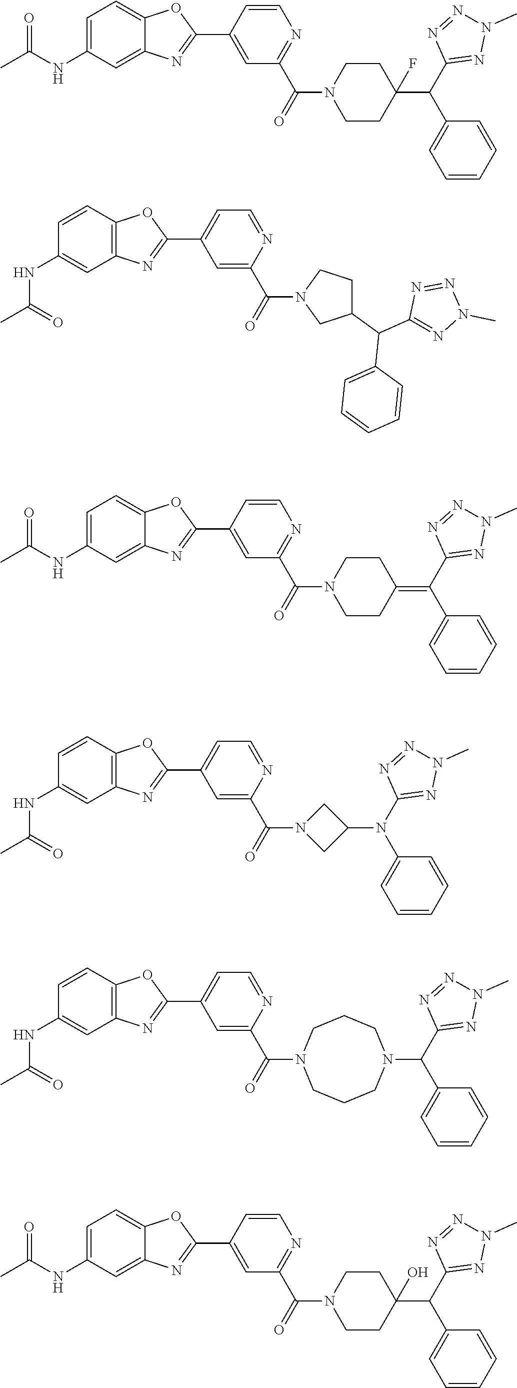

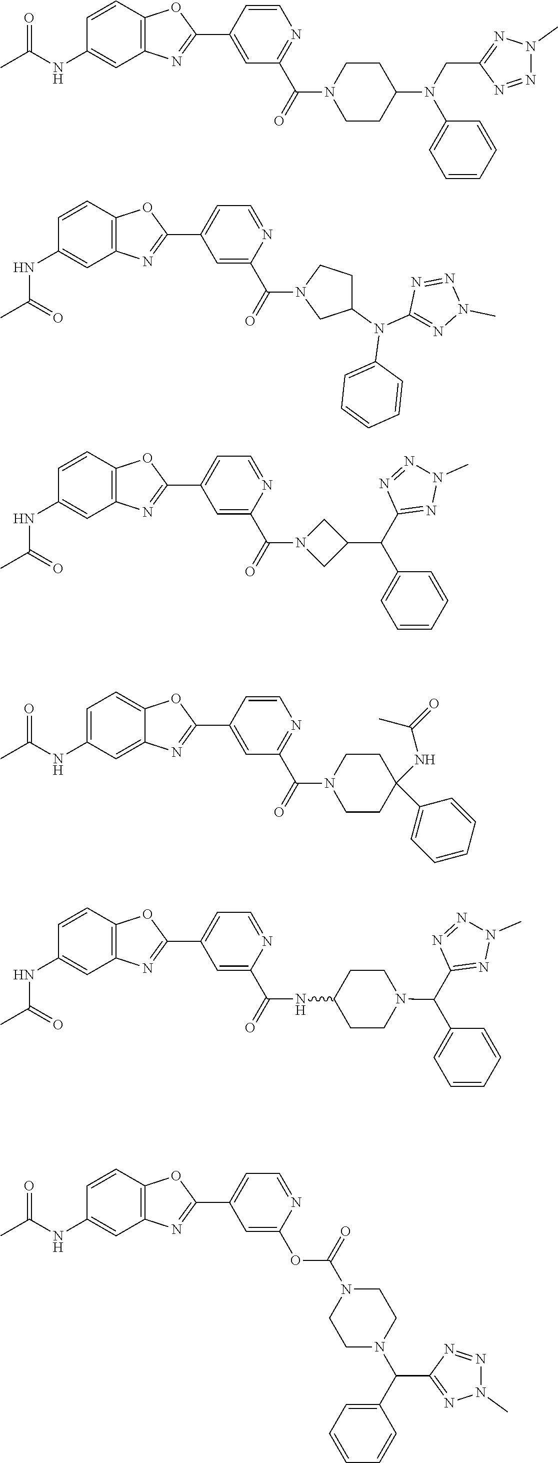

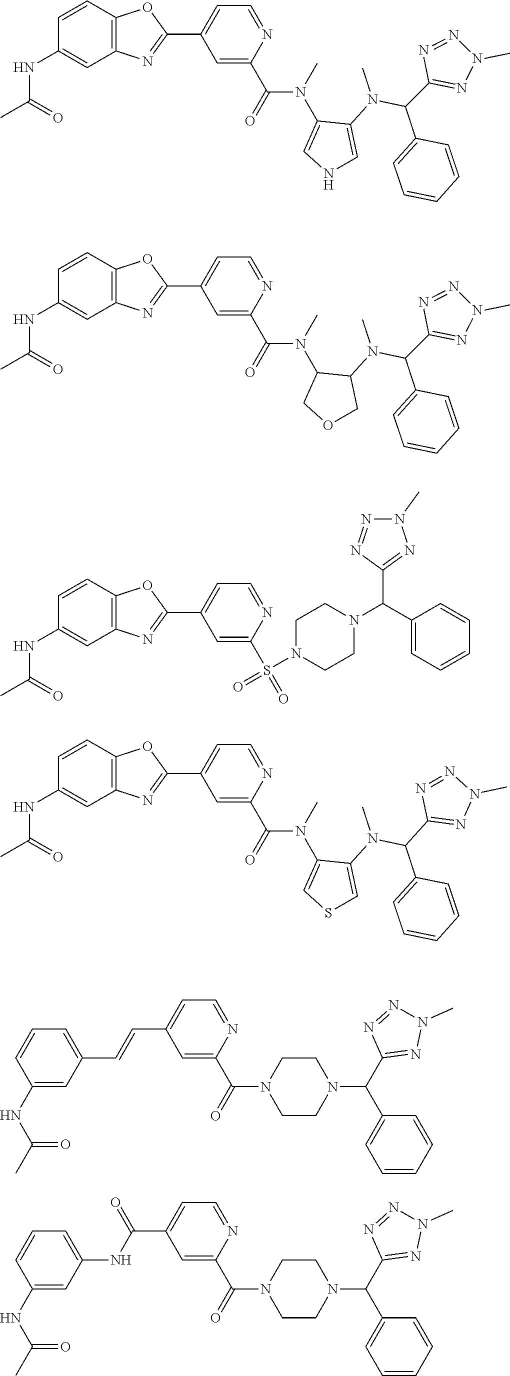

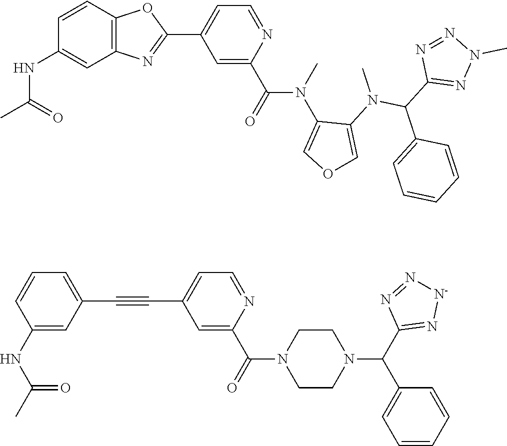 [SMILES: CC(=O)Nc1ccc2oc(-c3ccnc(C(=O)N(C)c4cocc4N(C)C(c4ccccc4)c4nnn(C)n4)c3)nc2c1 ; CC(=O)Nc1cccc(C#Cc2ccnc(C(=O)N3CCN(C(c4ccccc4)c4nn[n-]n4)CC3)c2)c1]

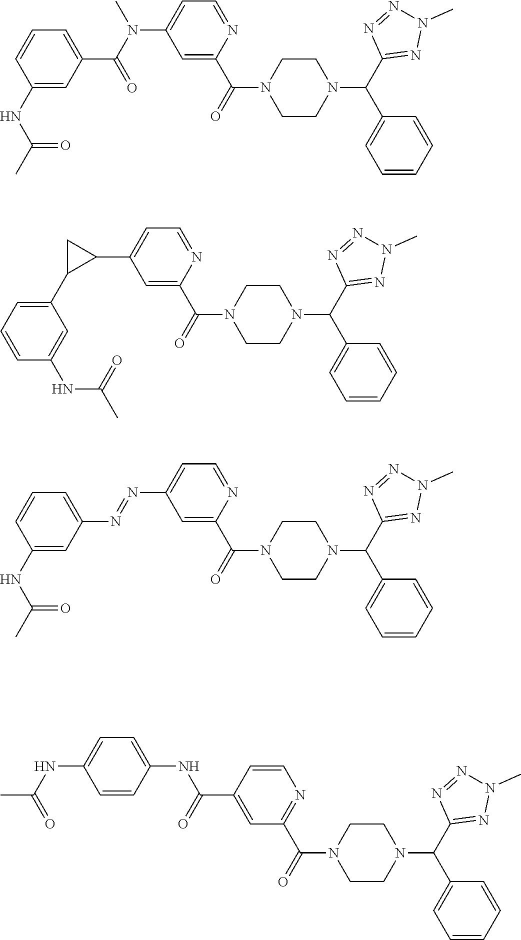

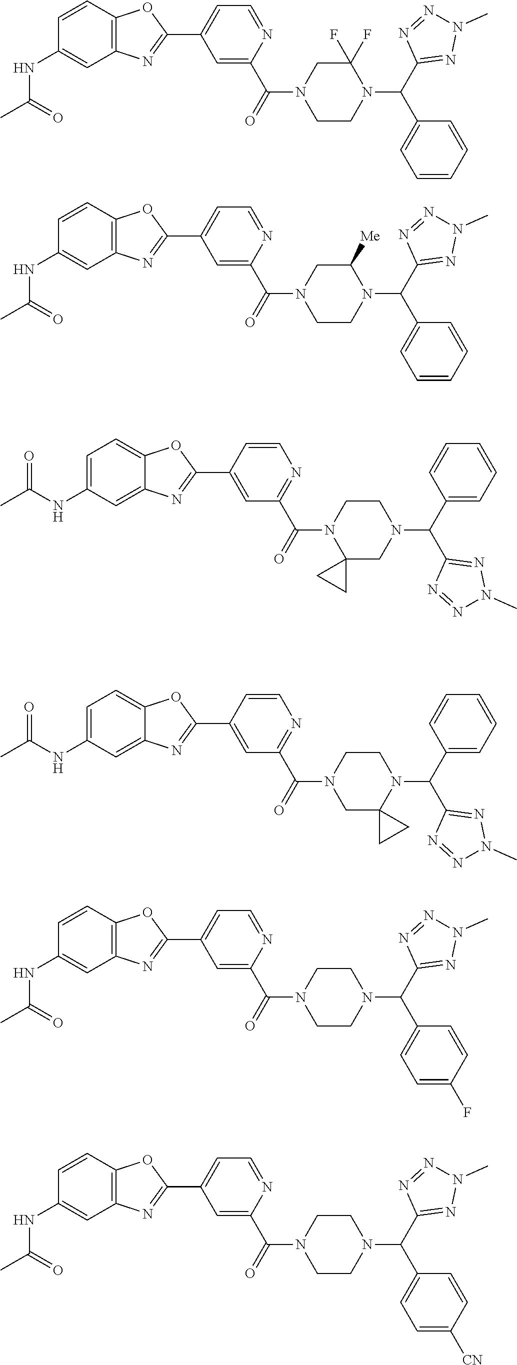

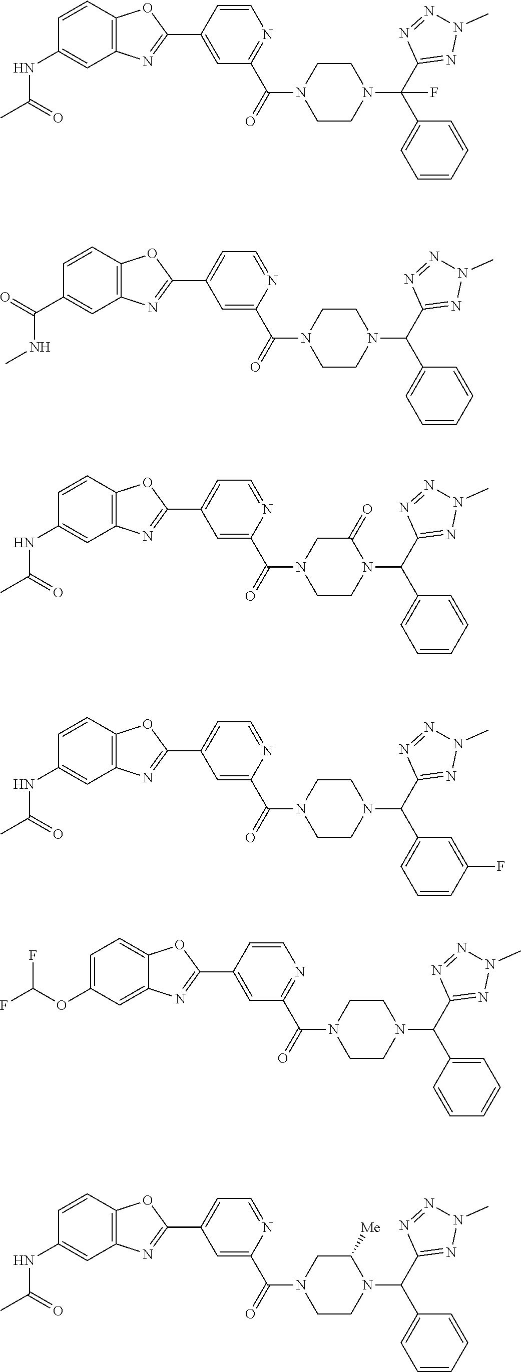

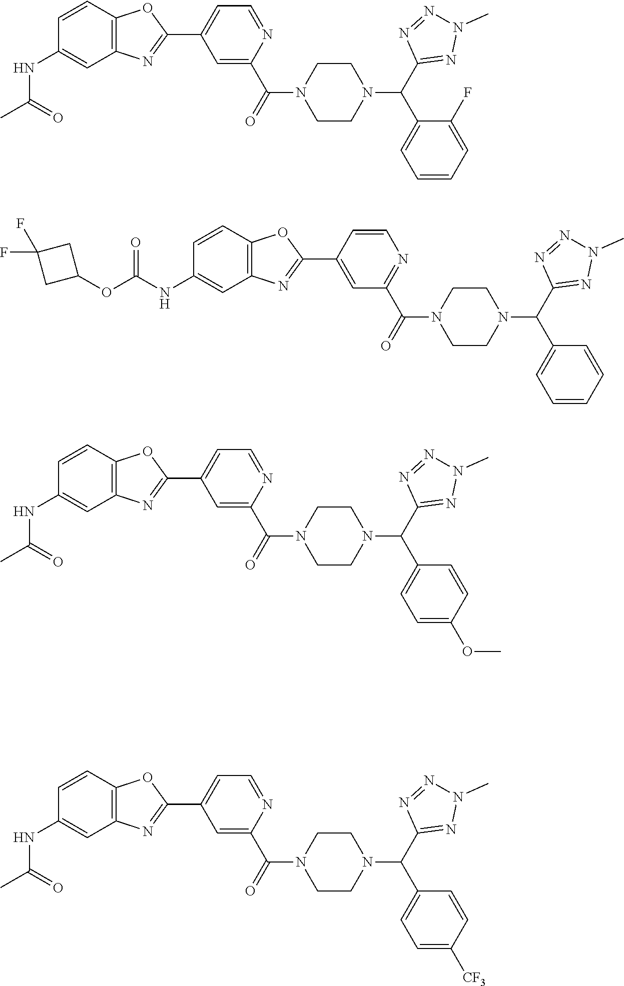

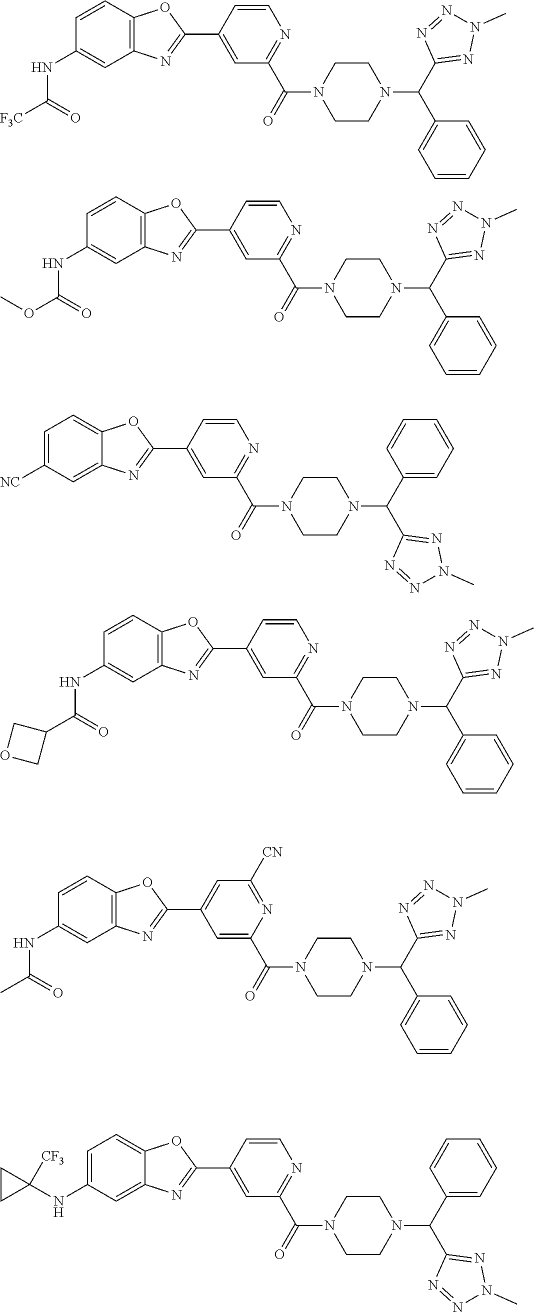

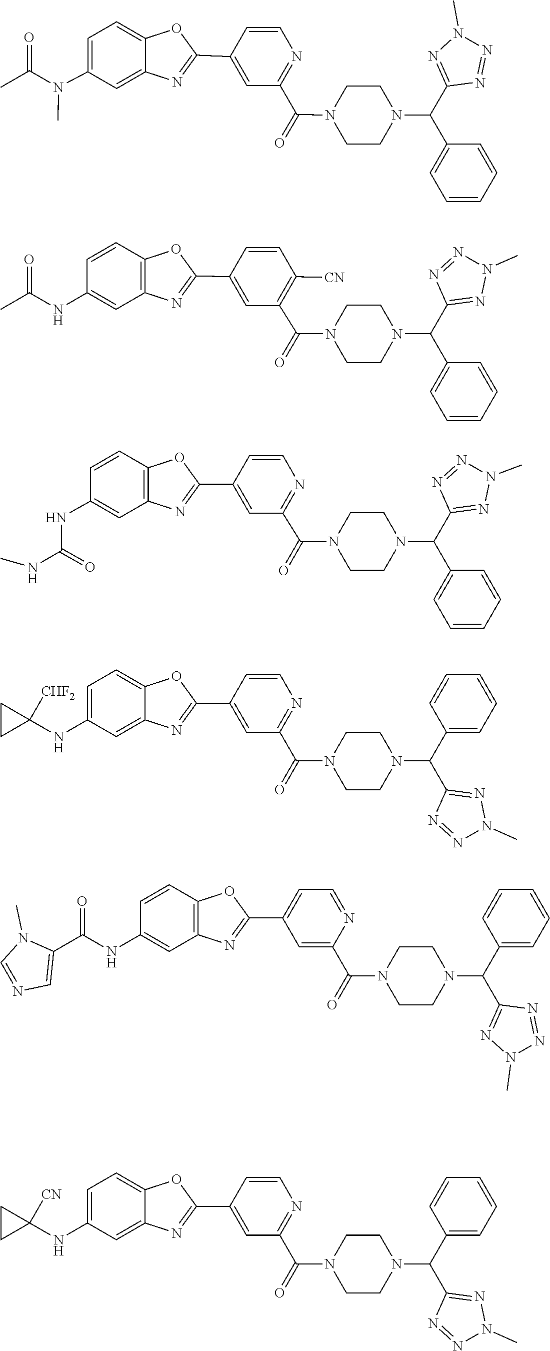

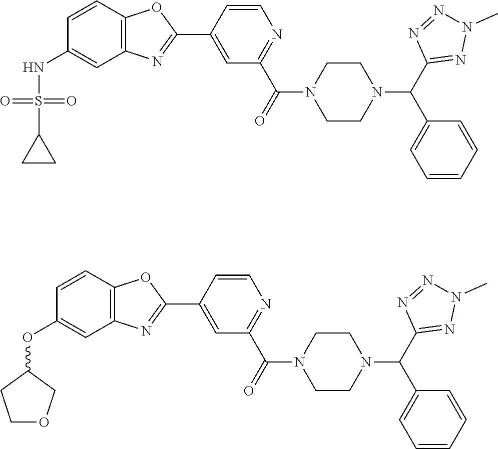 [SMILES: Cn1nnc(C(c2ccccc2)N2CCN(C(=O)c3cc(-c4nc5cc(NS(=O)(=O)C6CC6)ccc5o4)ccn3)CC2)n1 ; Cn1nnc(C(c2ccccc2)N2CCN(C(=O)c3cc(-c4nc5cc(OC6CCOC6)ccc5o4)ccn3)CC2)n1]

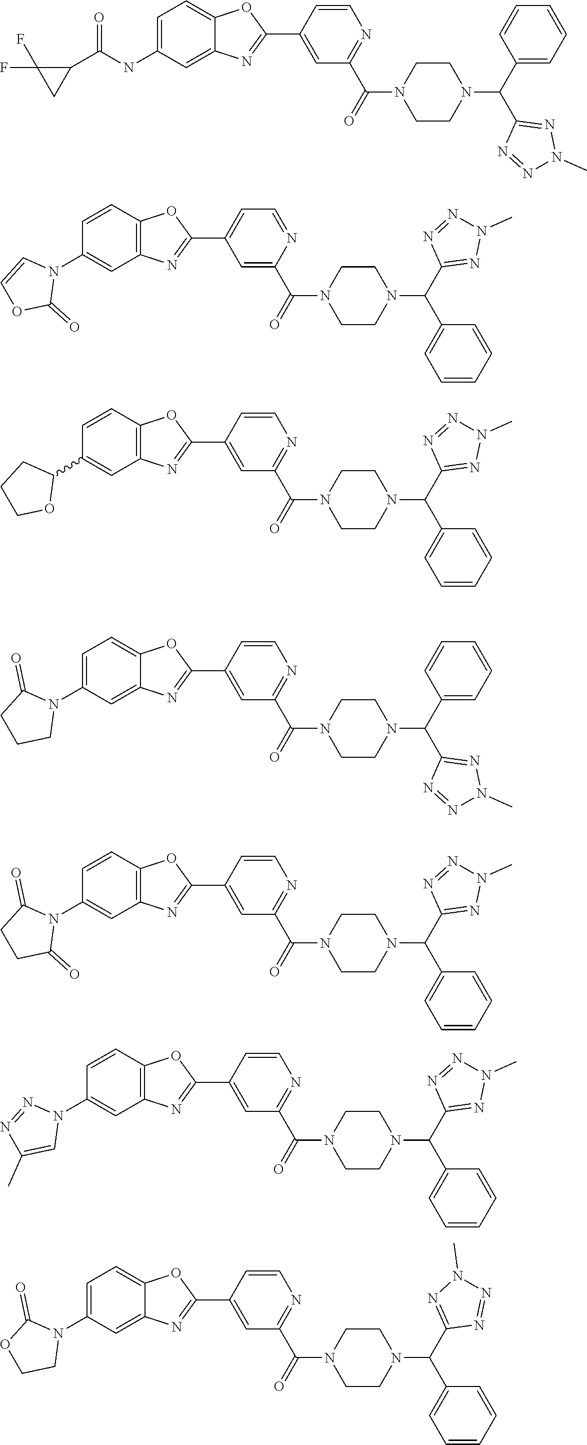

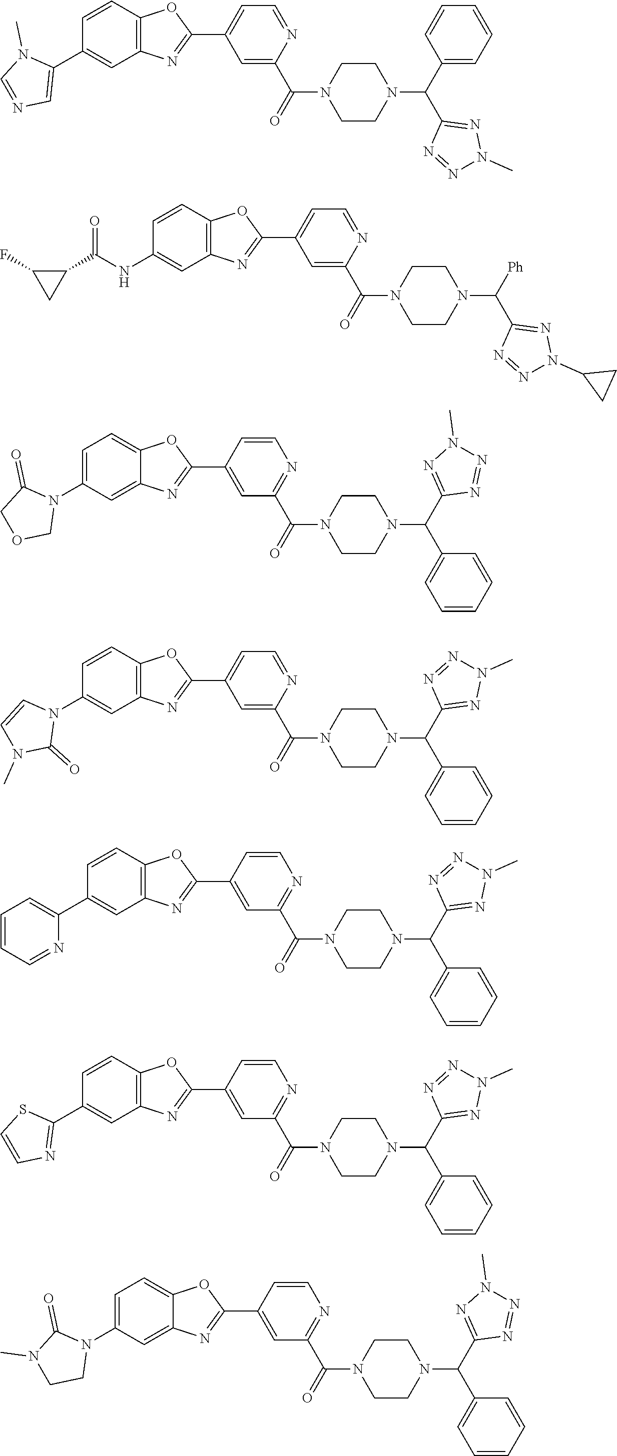

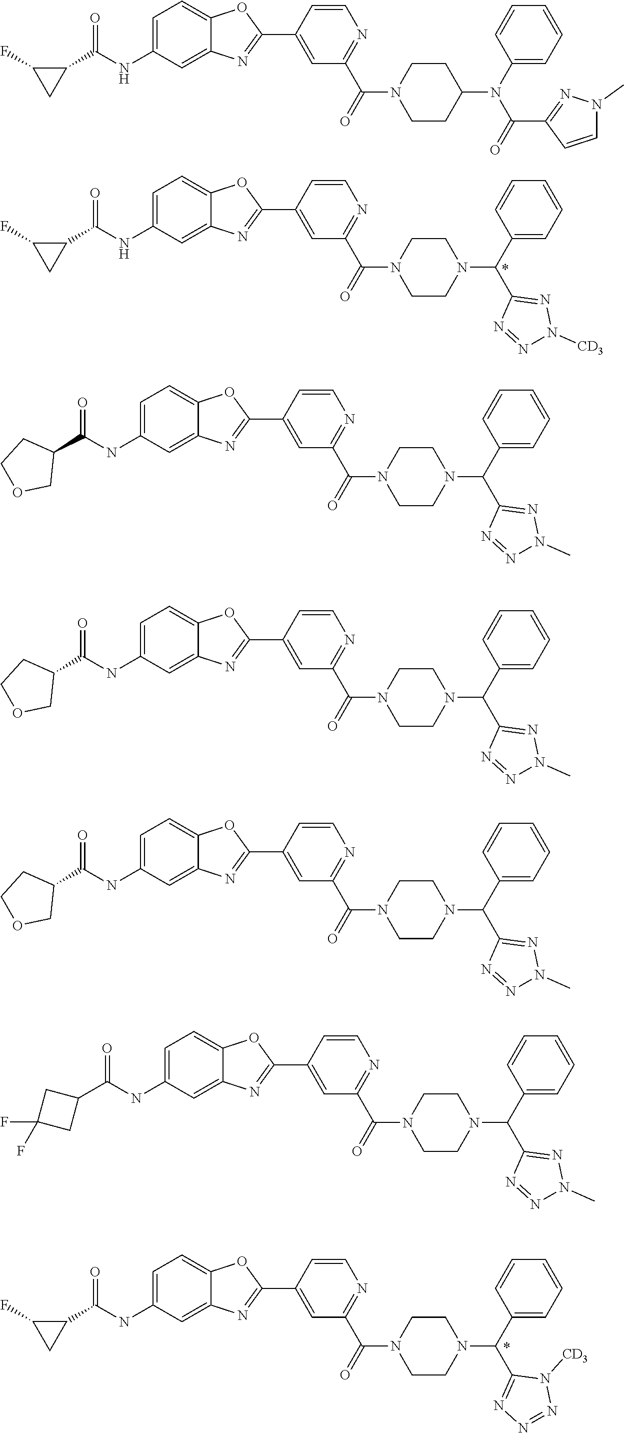

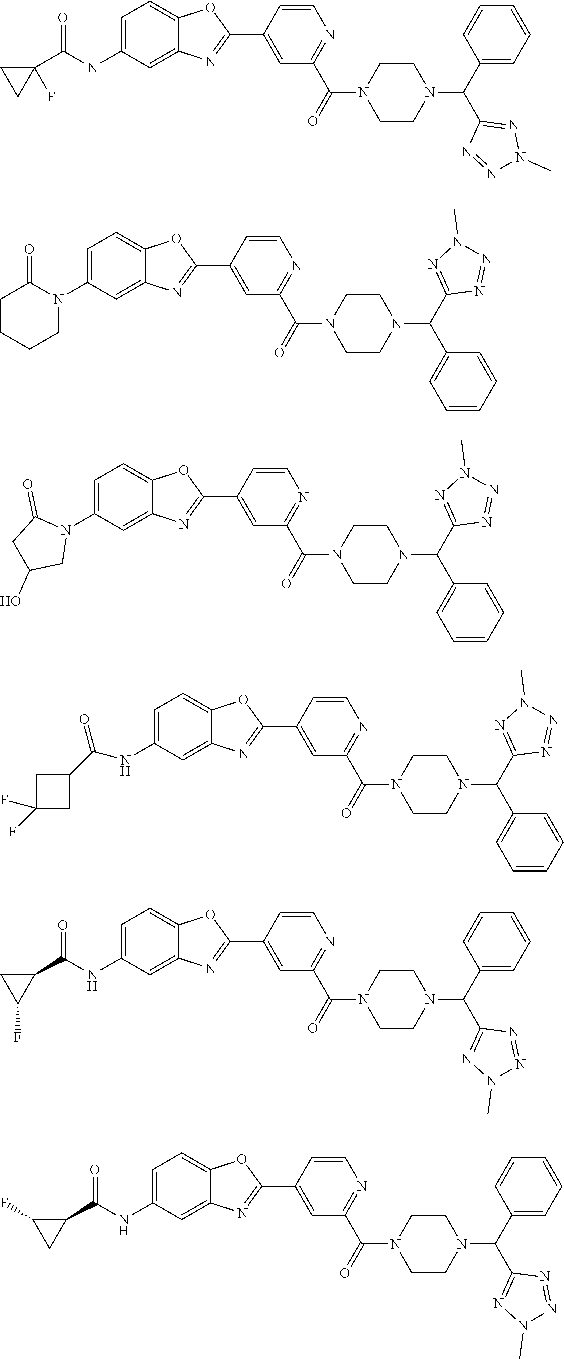

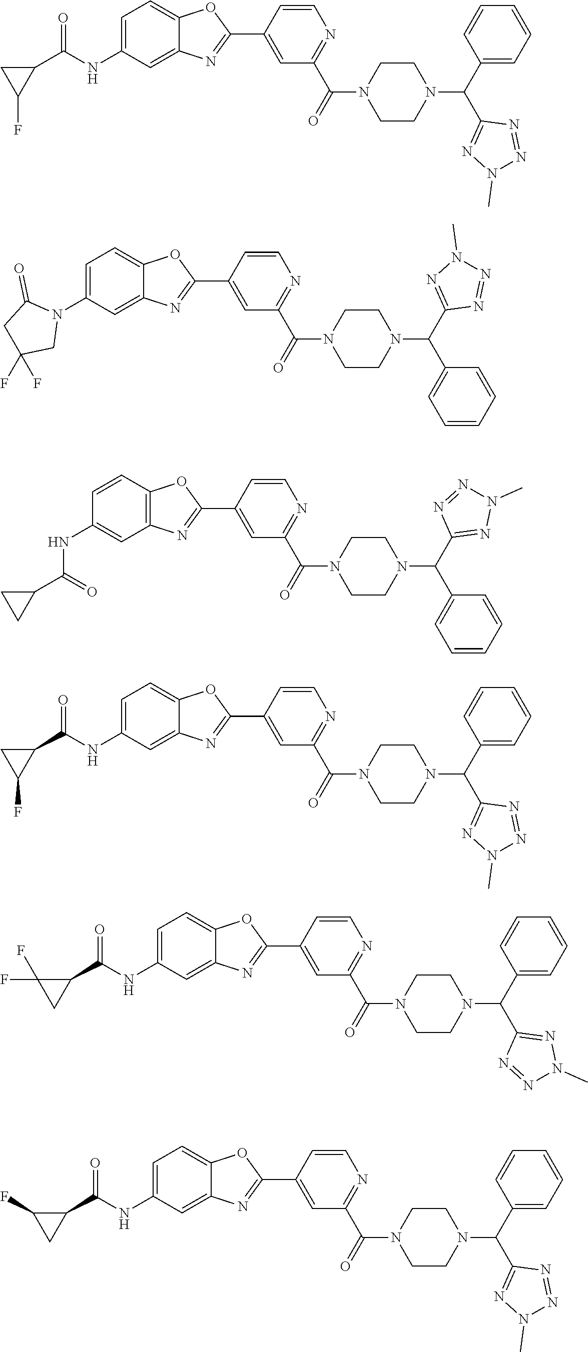

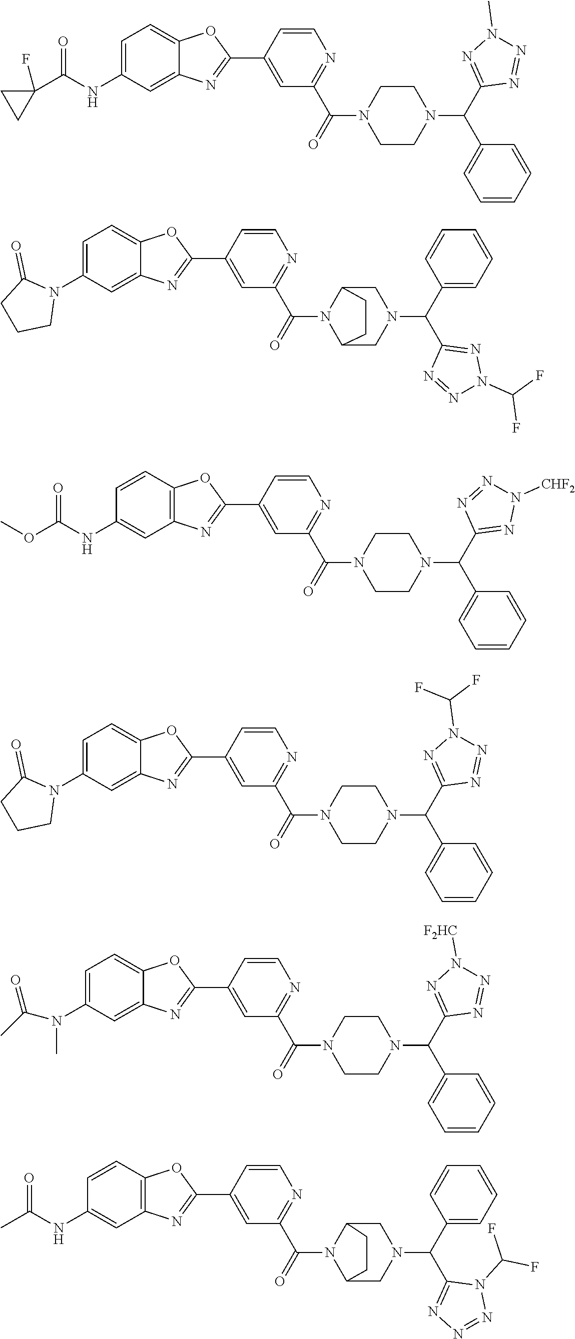

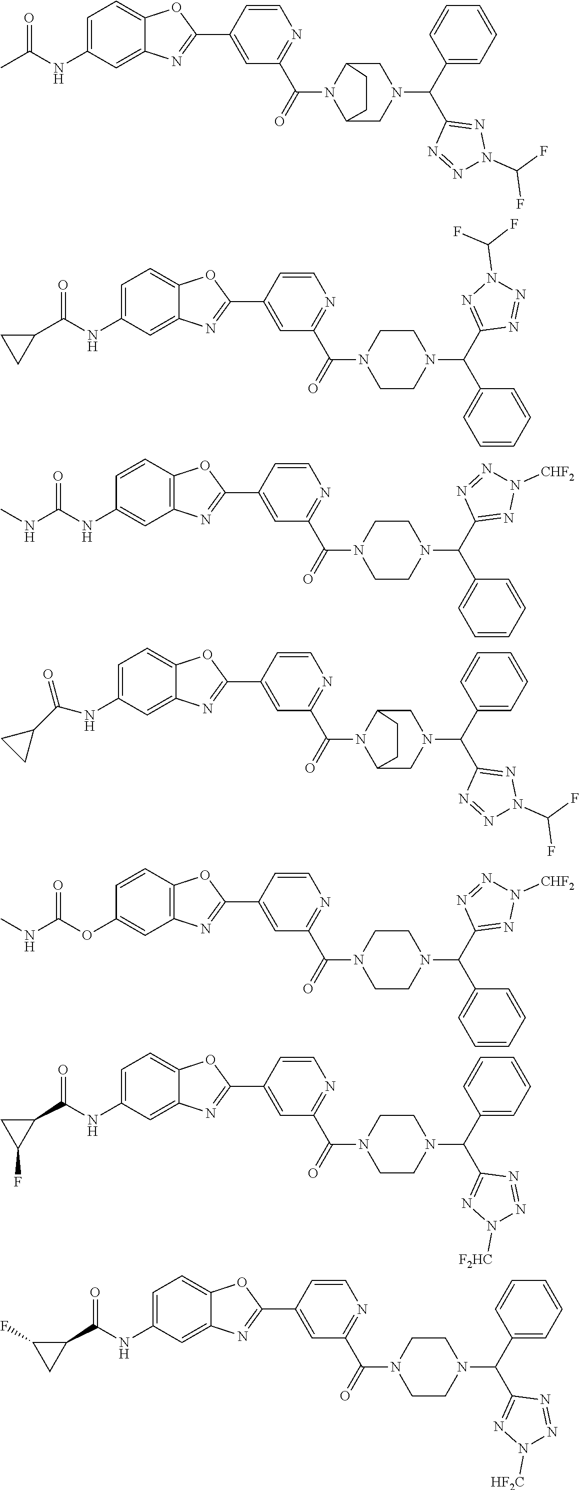

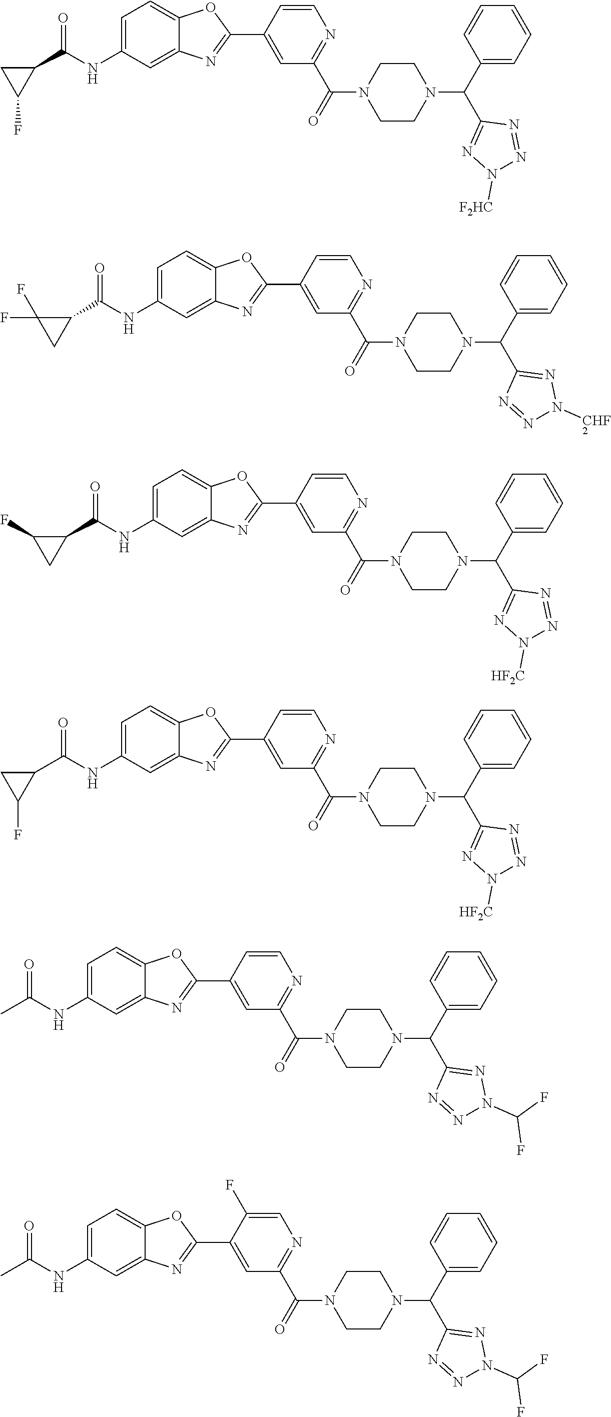

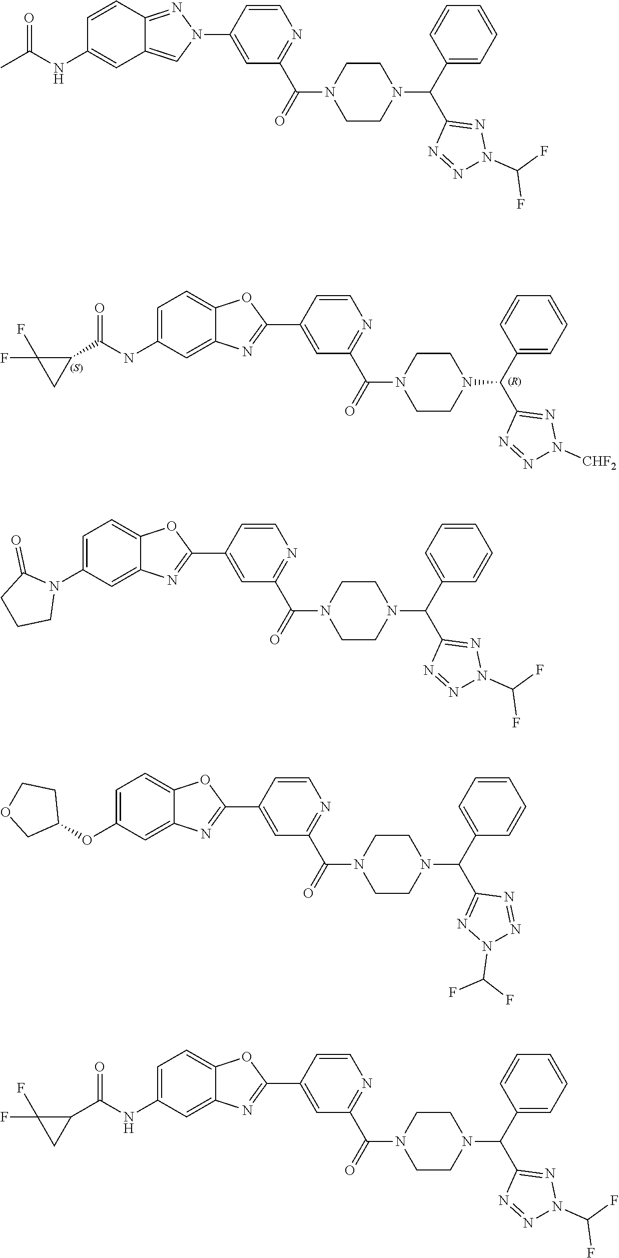

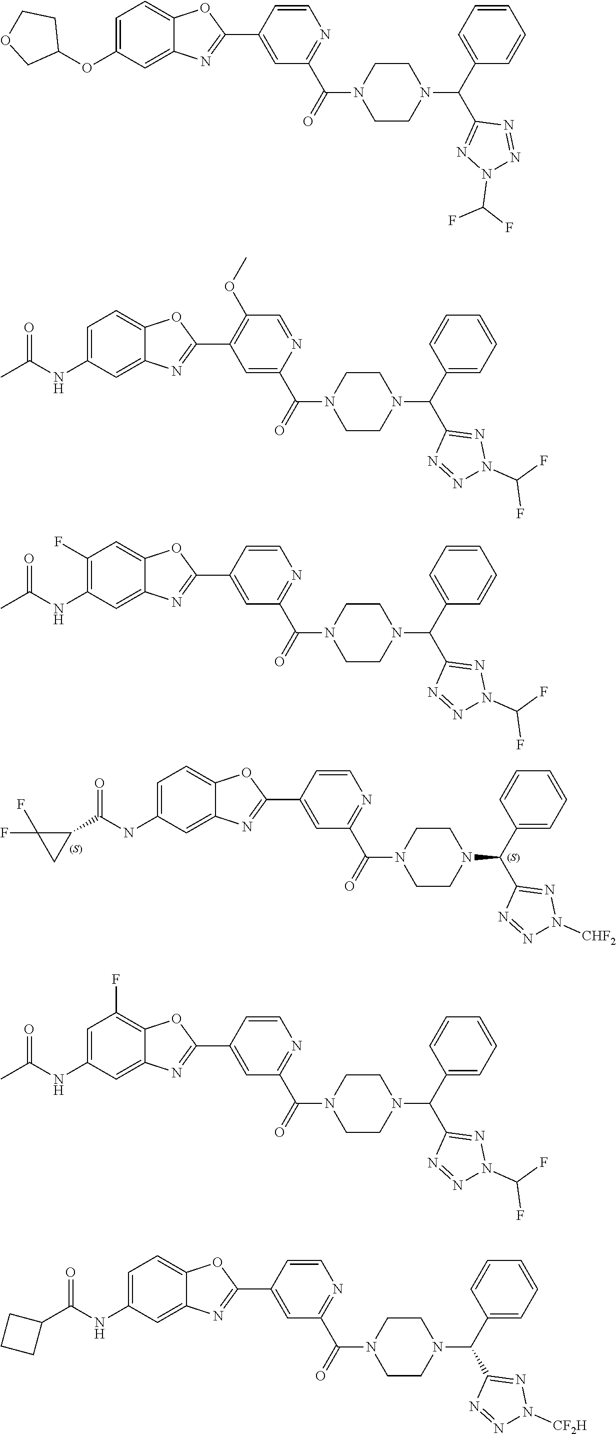

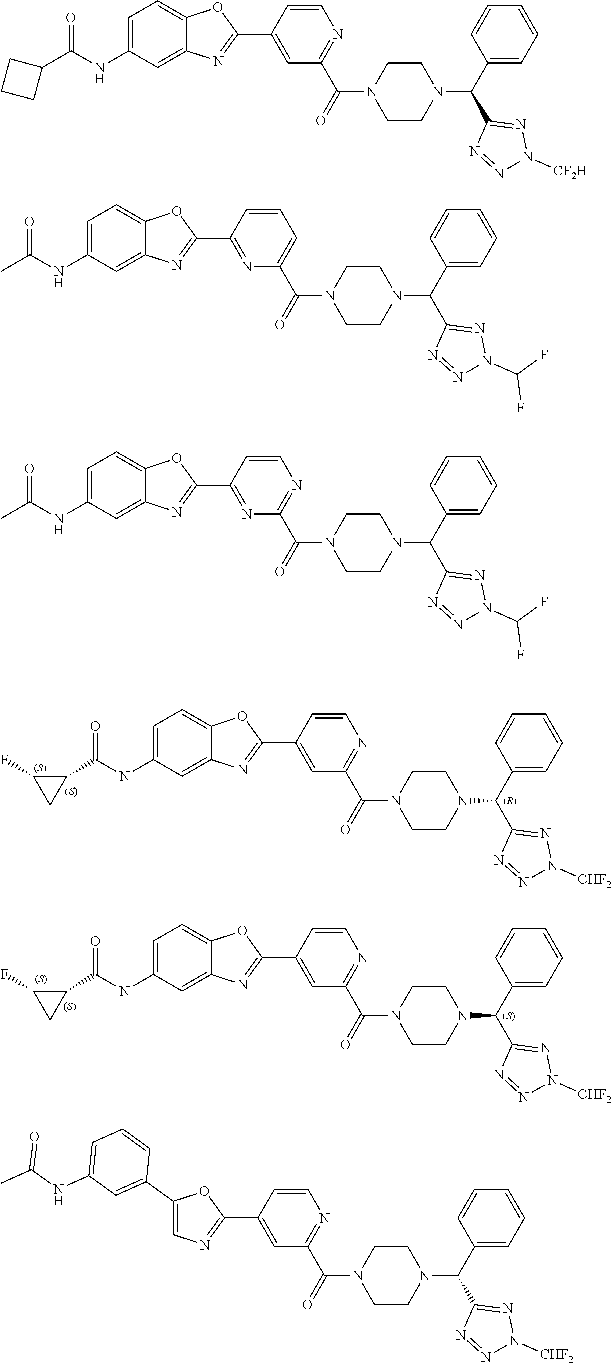

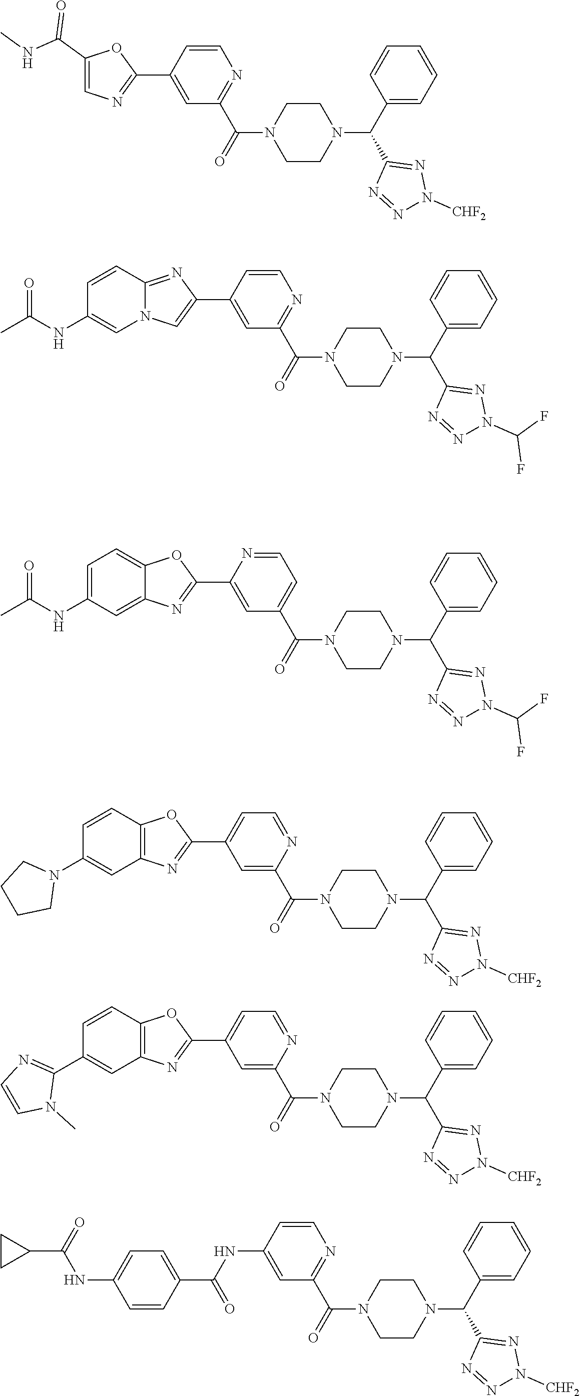

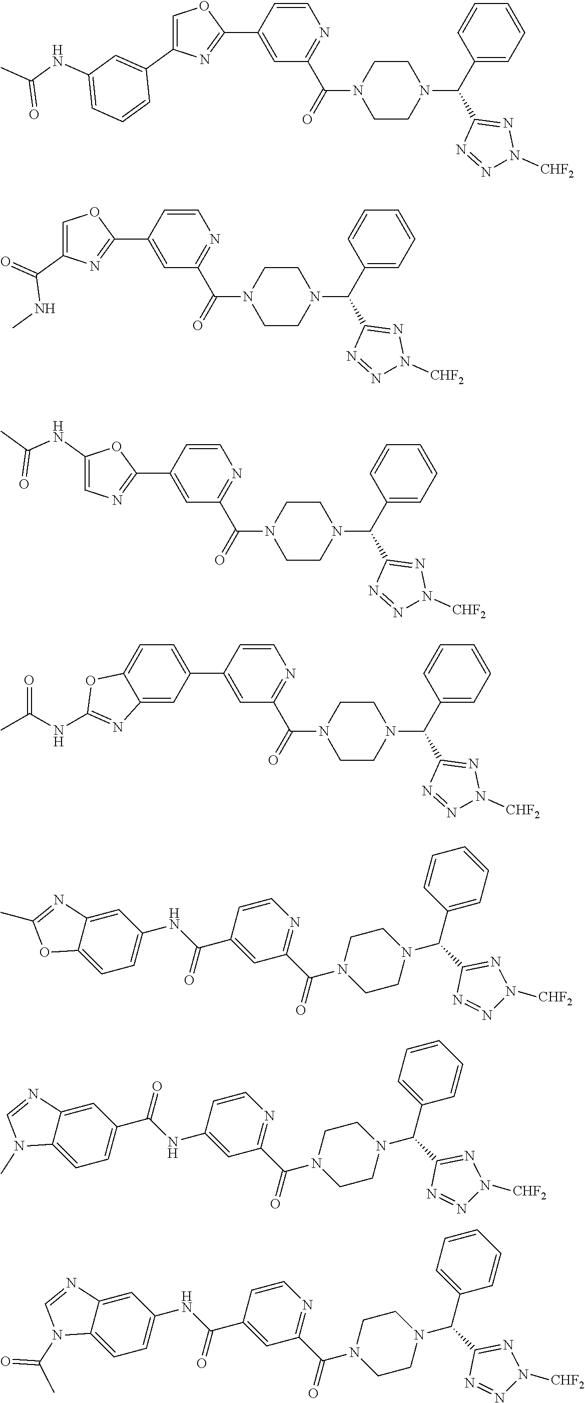

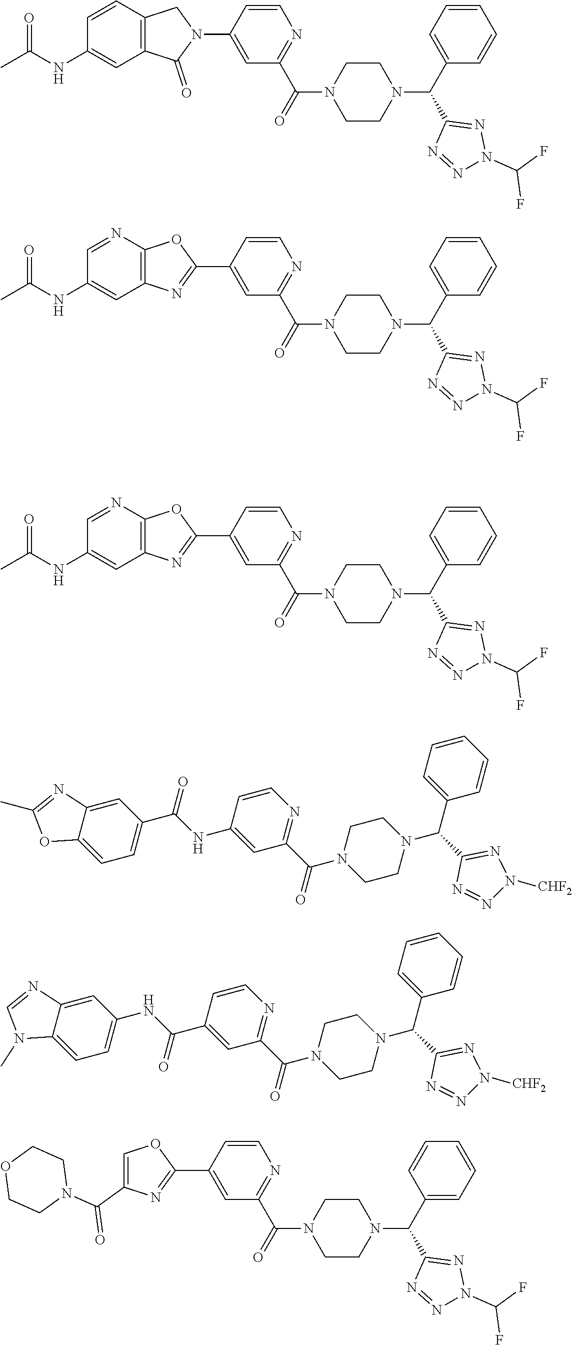

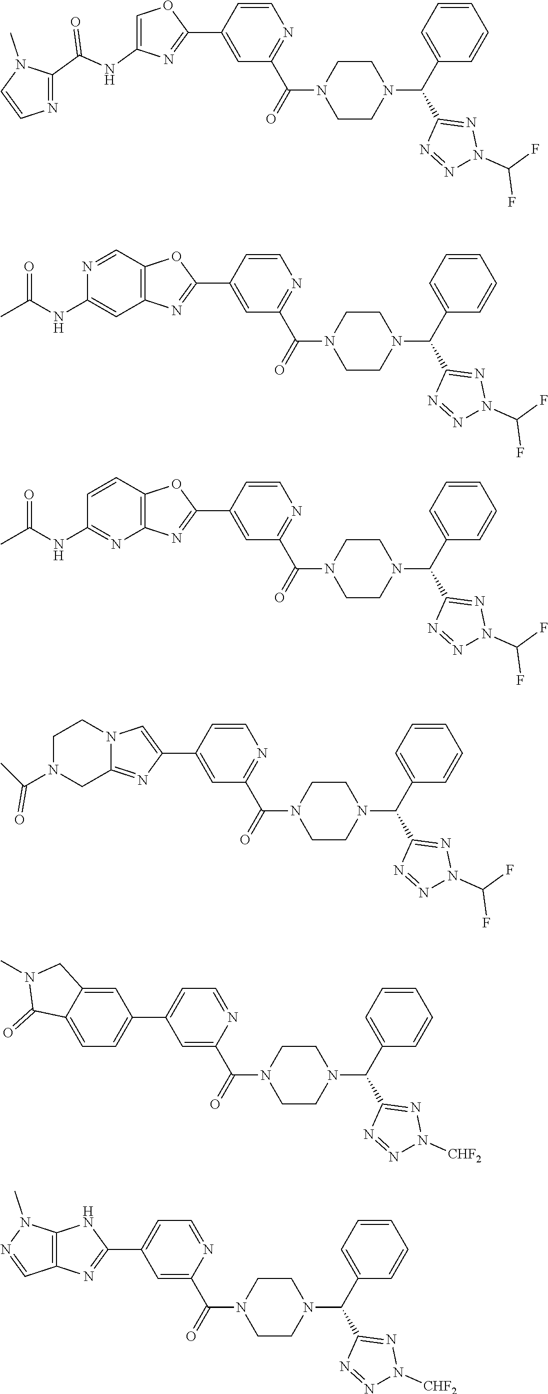 [SMILES: Cn1ccnc1C(=O)Nc1coc(-c2ccnc(C(=O)N3CCN([C@H](c4ccccc4)c4nnn(C(F)F)n4)CC3)c2)n1 ; CC(=O)Nc1cc2nc(-c3ccnc(C(=O)N4CCN([C@H](c5ccccc5)c5nnn(C(F)F)n5)CC4)c3)oc2cn1 ; CC(=O)Nc1ccc2oc(-c3ccnc(C(=O)N4CCN([C@H](c5ccccc5)c5nnn(C(F)F)n5)CC4)c3)nc2n1 ; CC(=O)N1CCn2cc(-c3ccnc(C(=O)N4CCN([C@H](c5ccccc5)c5nnn(C(F)F)n5)CC4)c3)nc2C1 ; CN1Cc2cc(-c3ccnc(C(=O)N4CCN([C@H](c5ccccc5)c5nnn(C(F)F)n5)CC4)c3)ccc2C1=O ; Cn1ncc2nc(-c3ccnc(C(=O)N4CCN([C@H](c5ccccc5)c5nnn(C(F)F)n5)CC4)c3)[nH]c21]

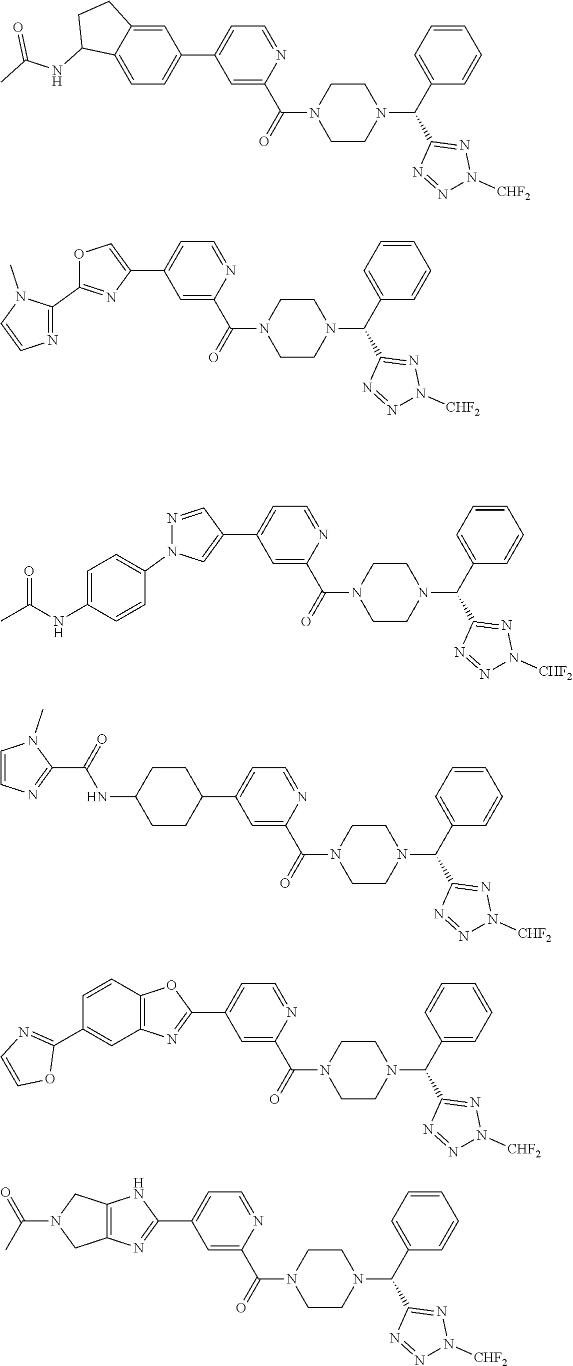

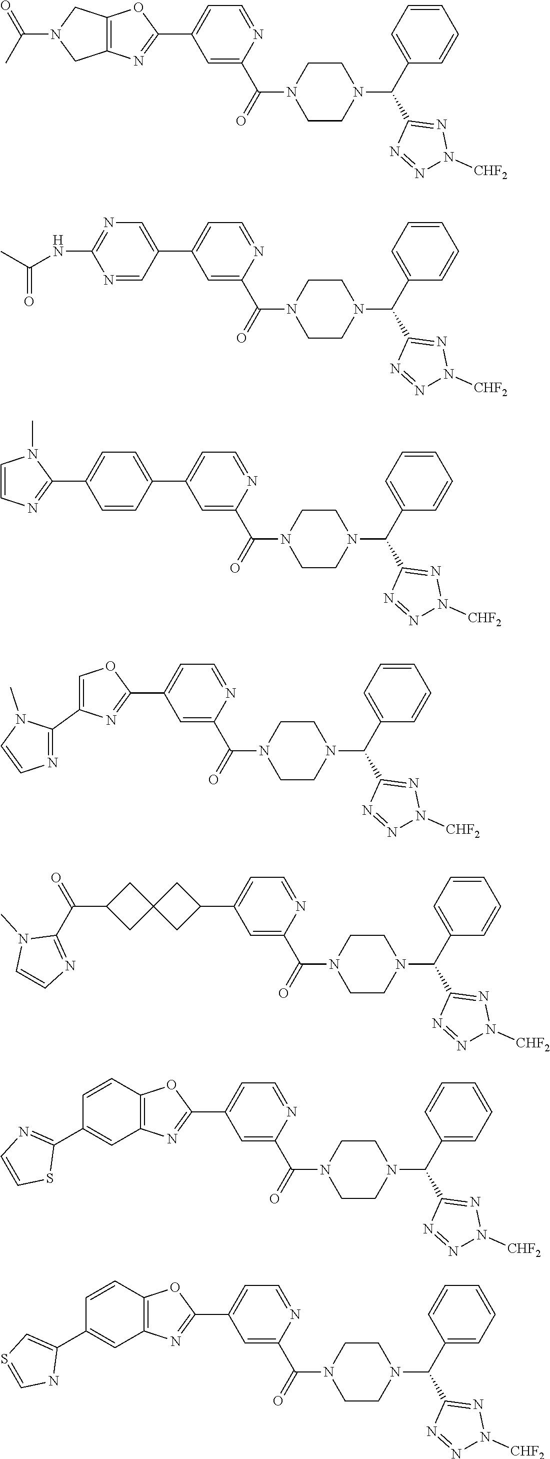

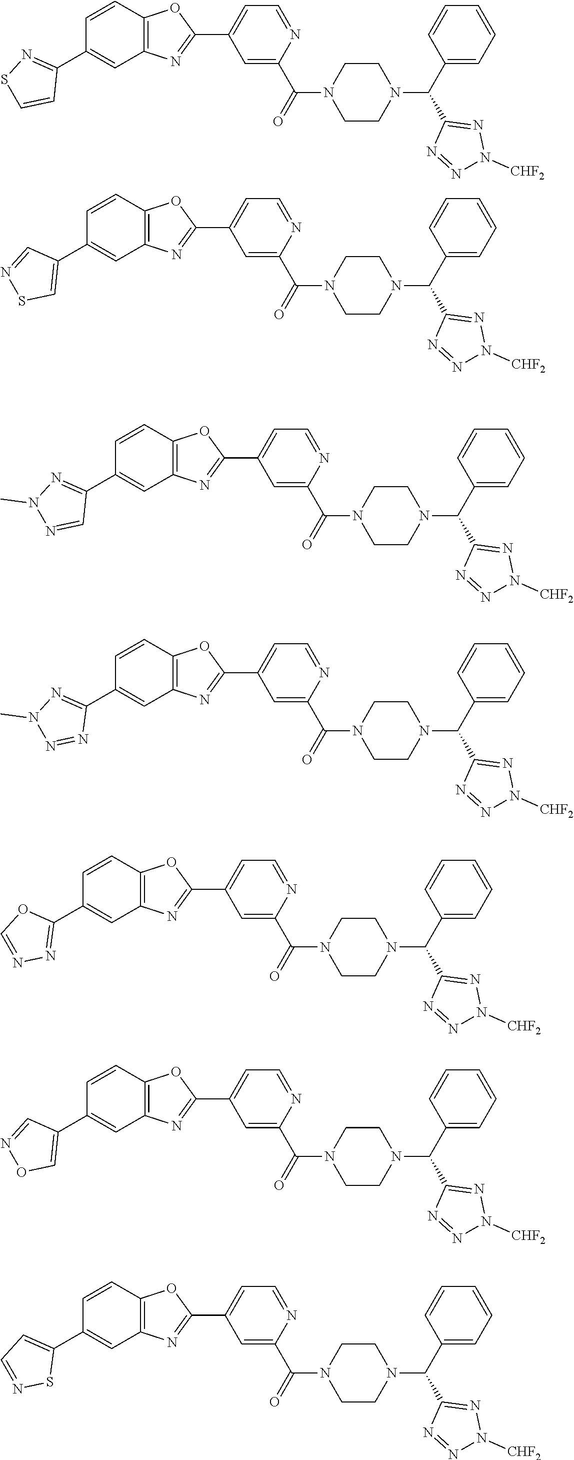

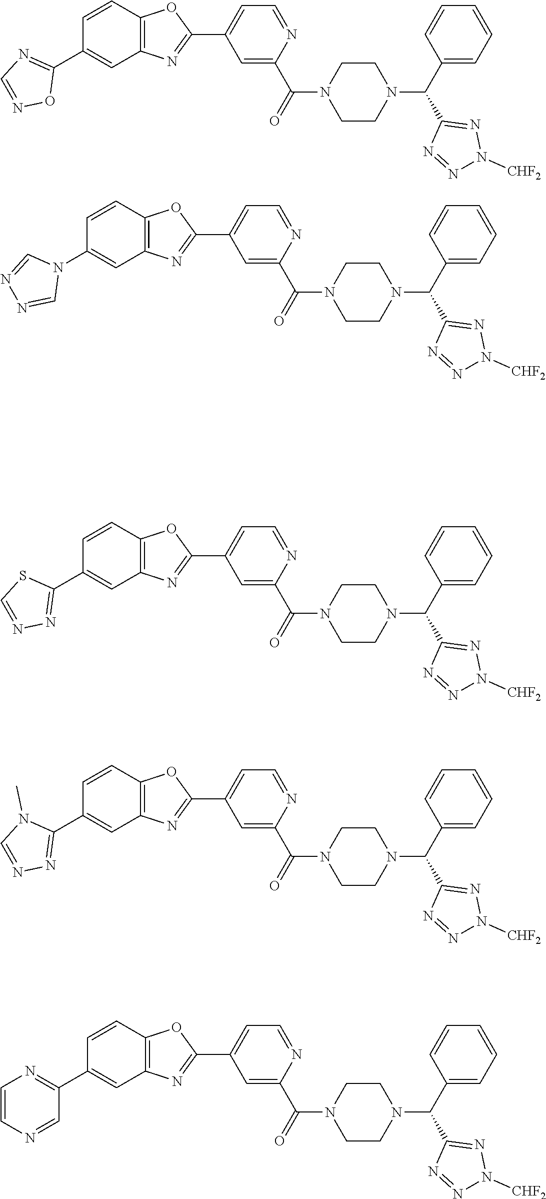

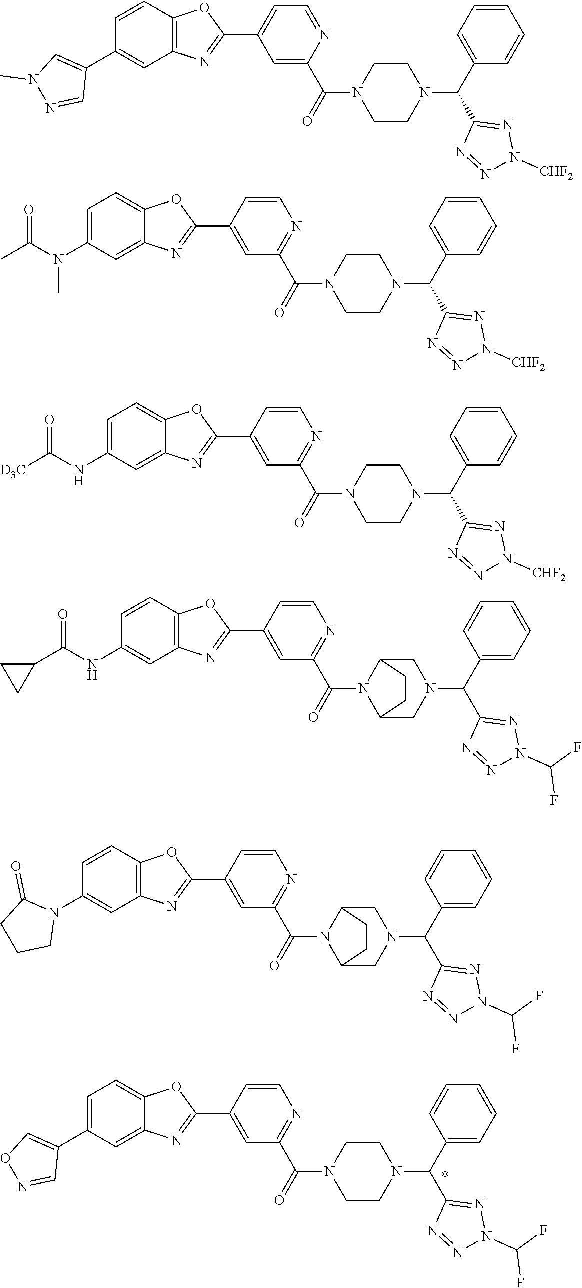

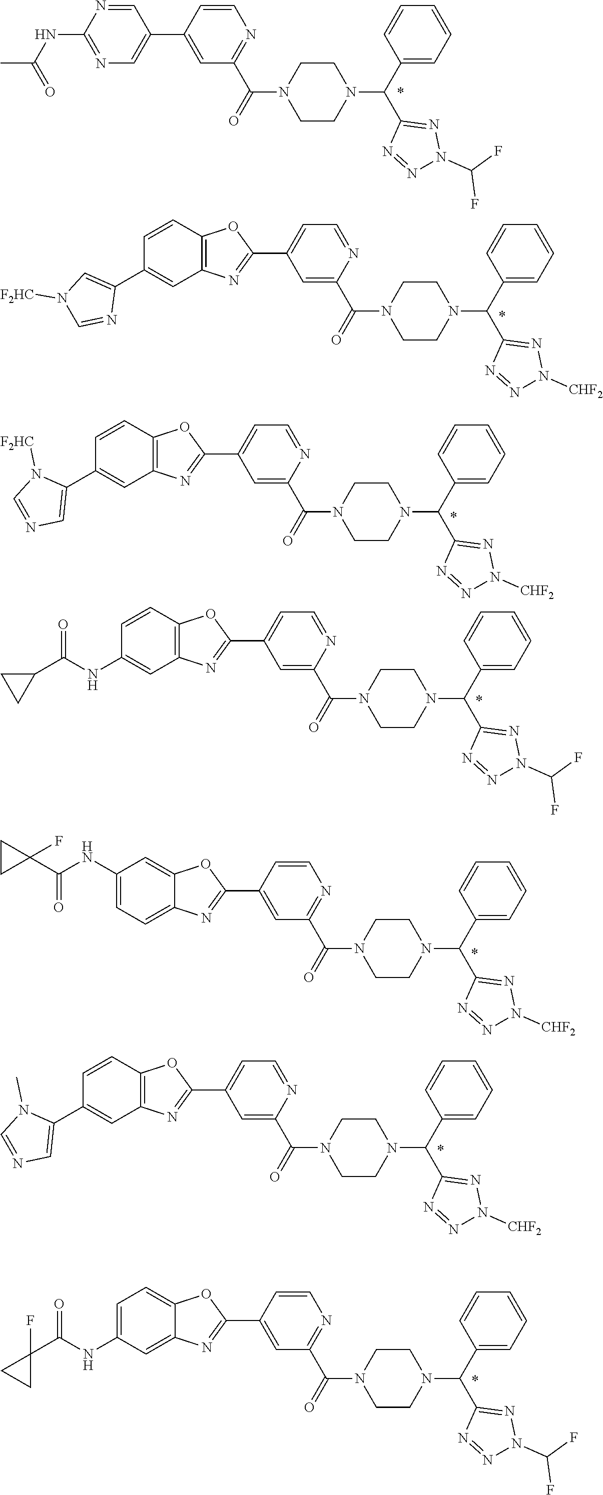

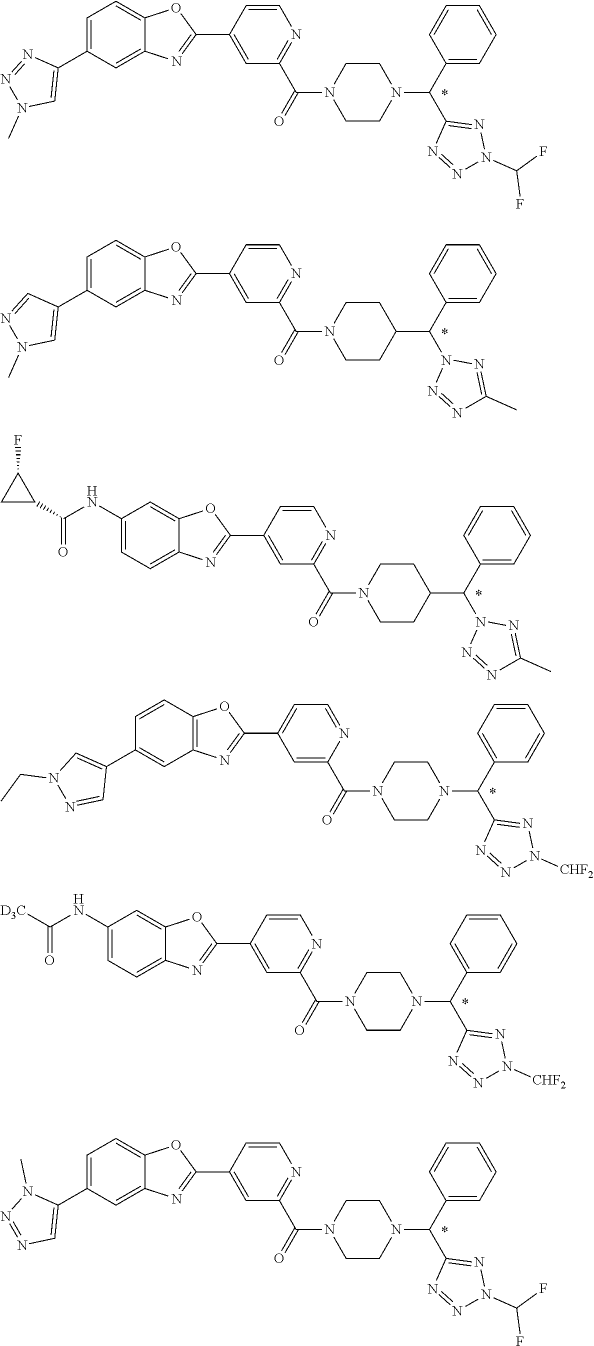

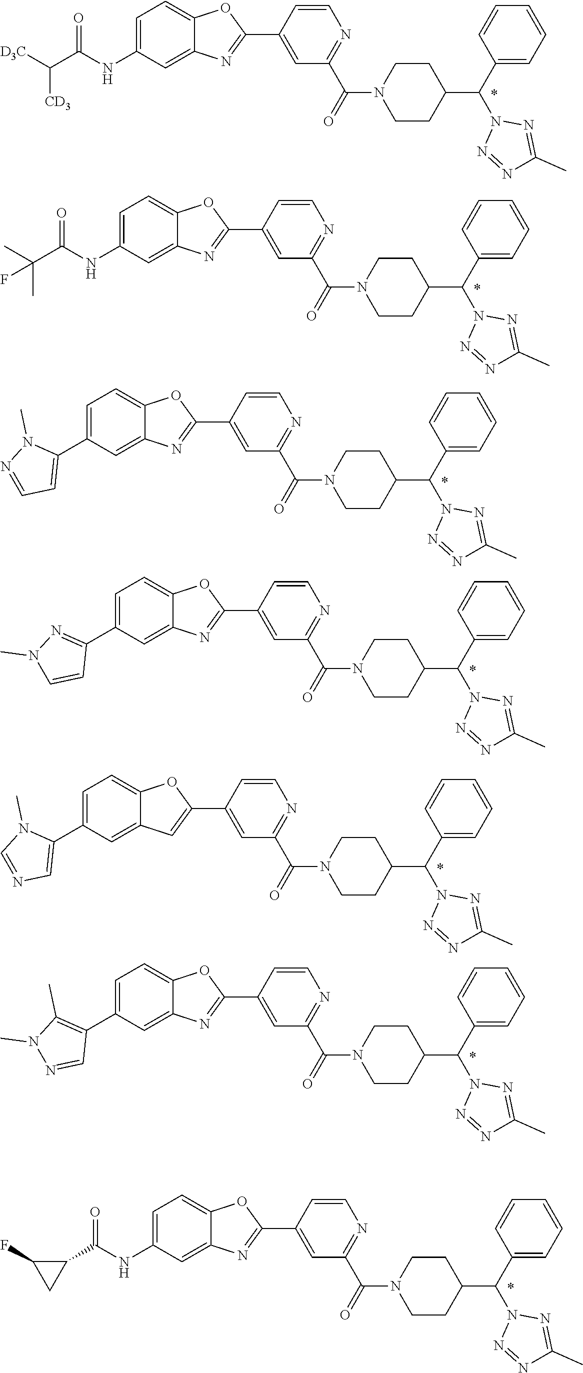

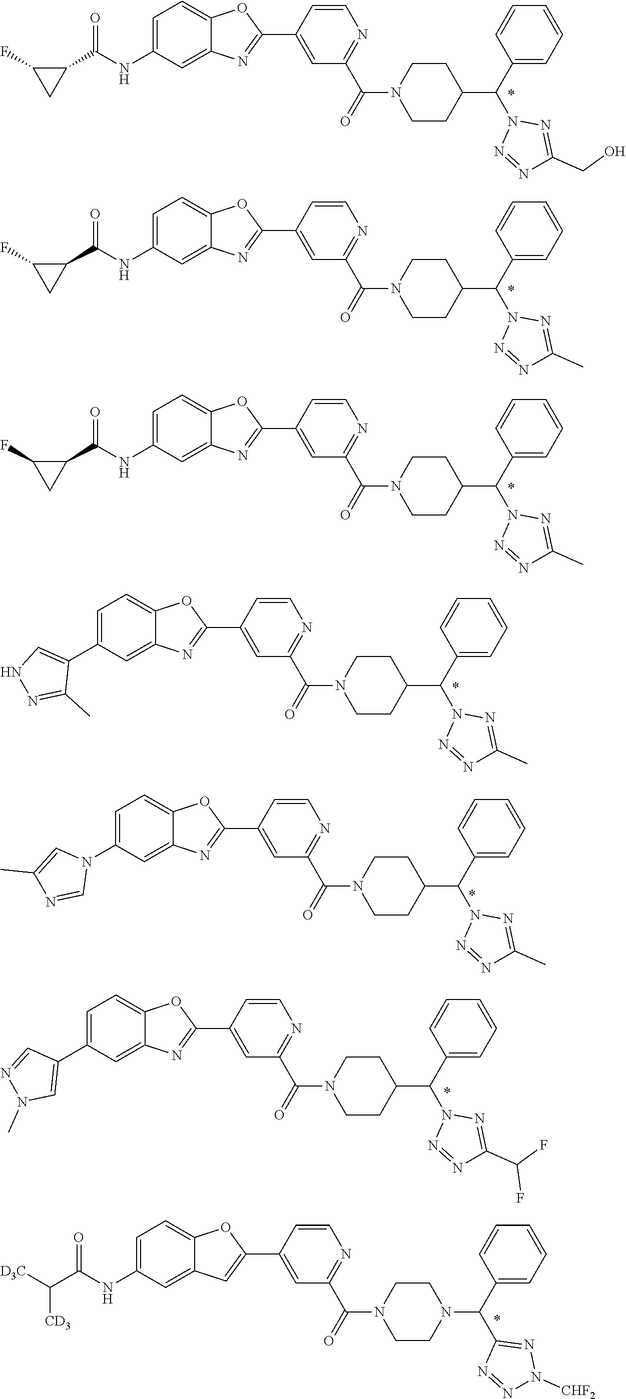

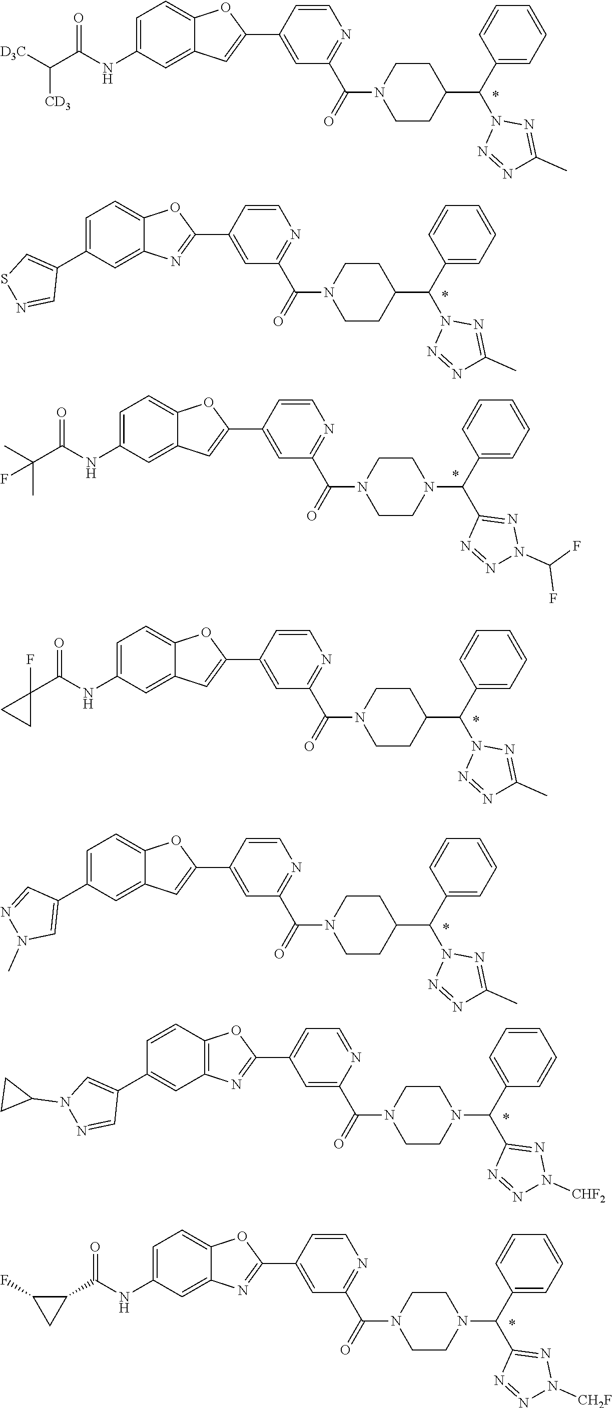

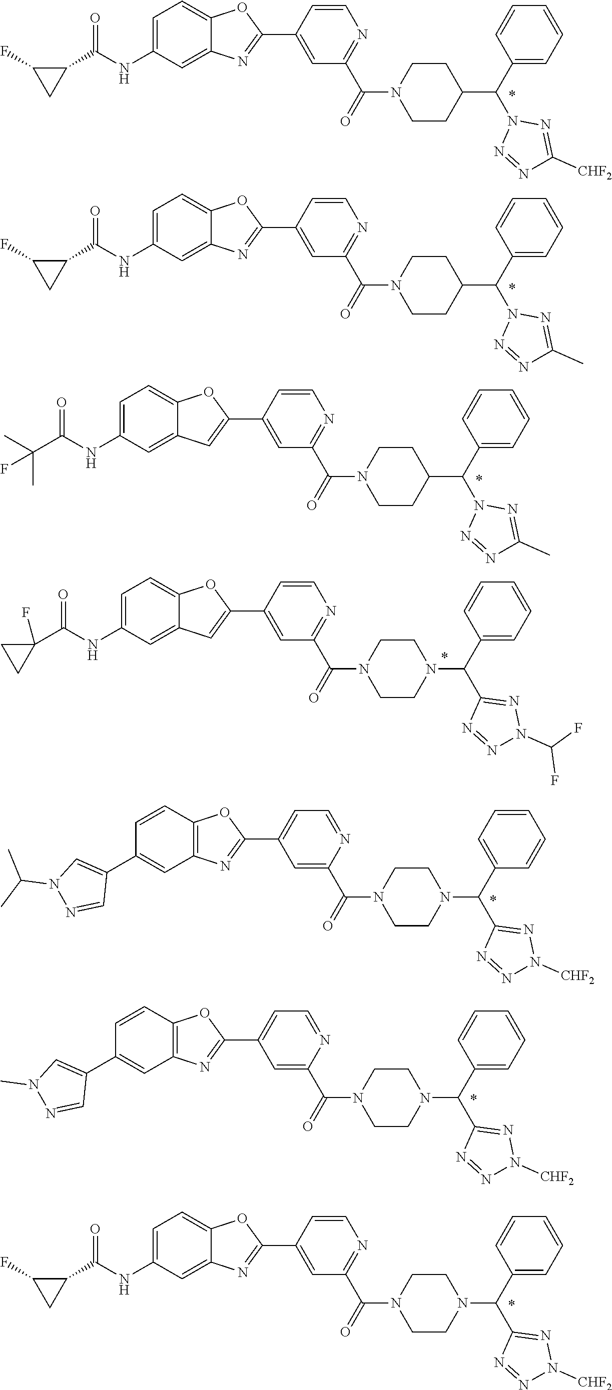

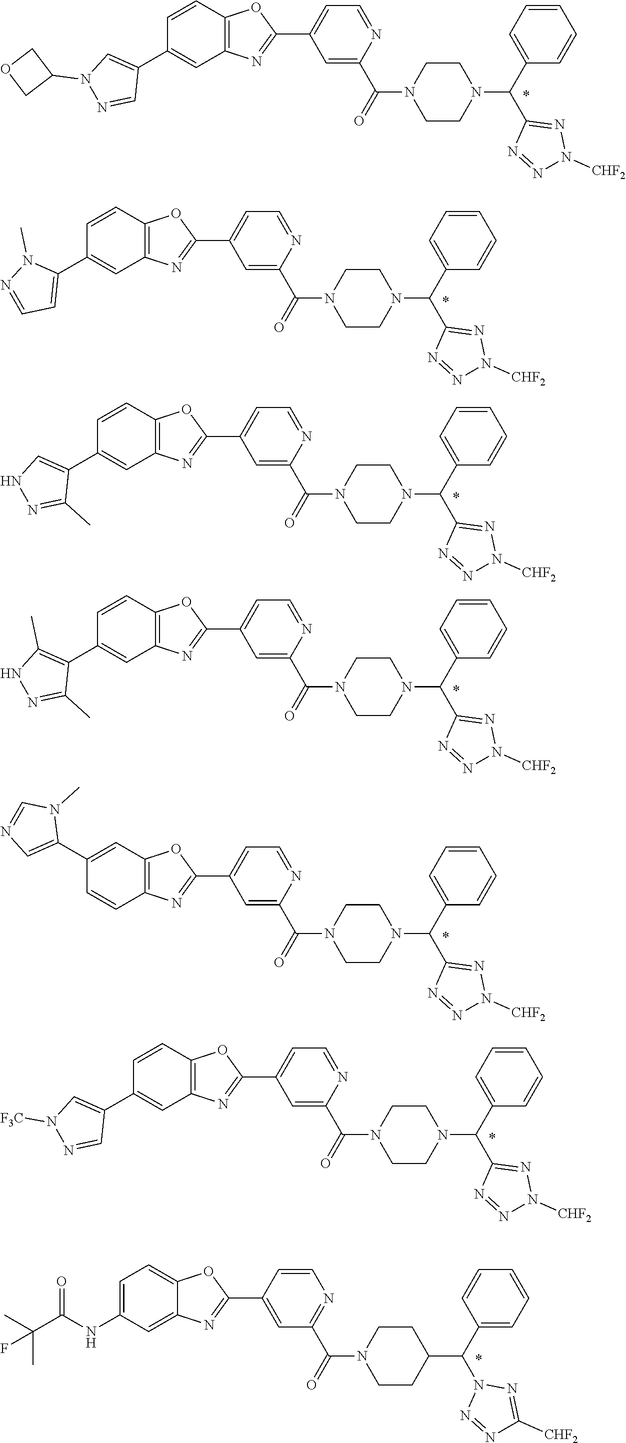

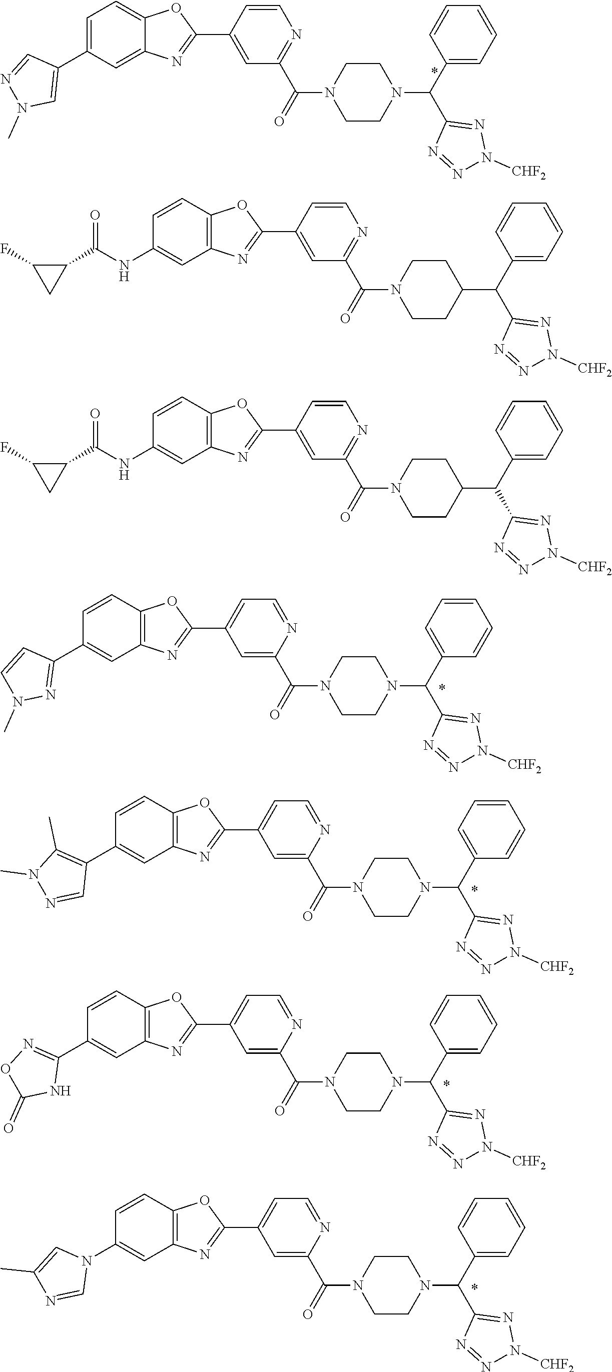

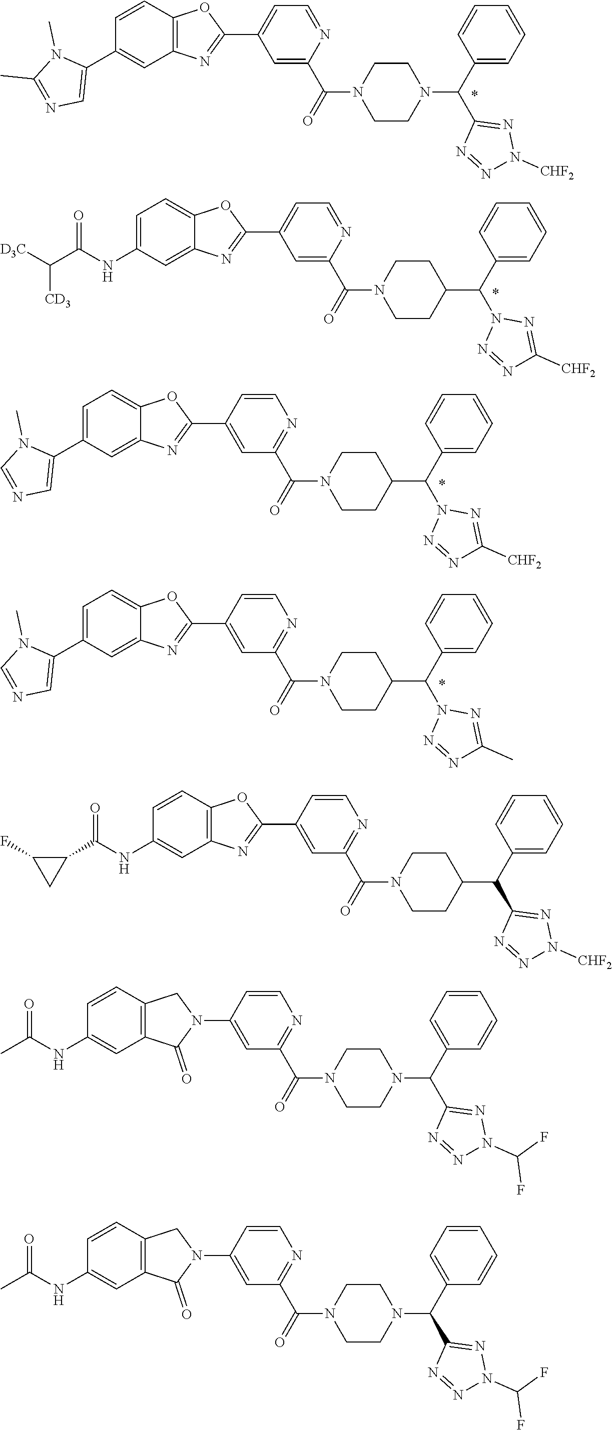

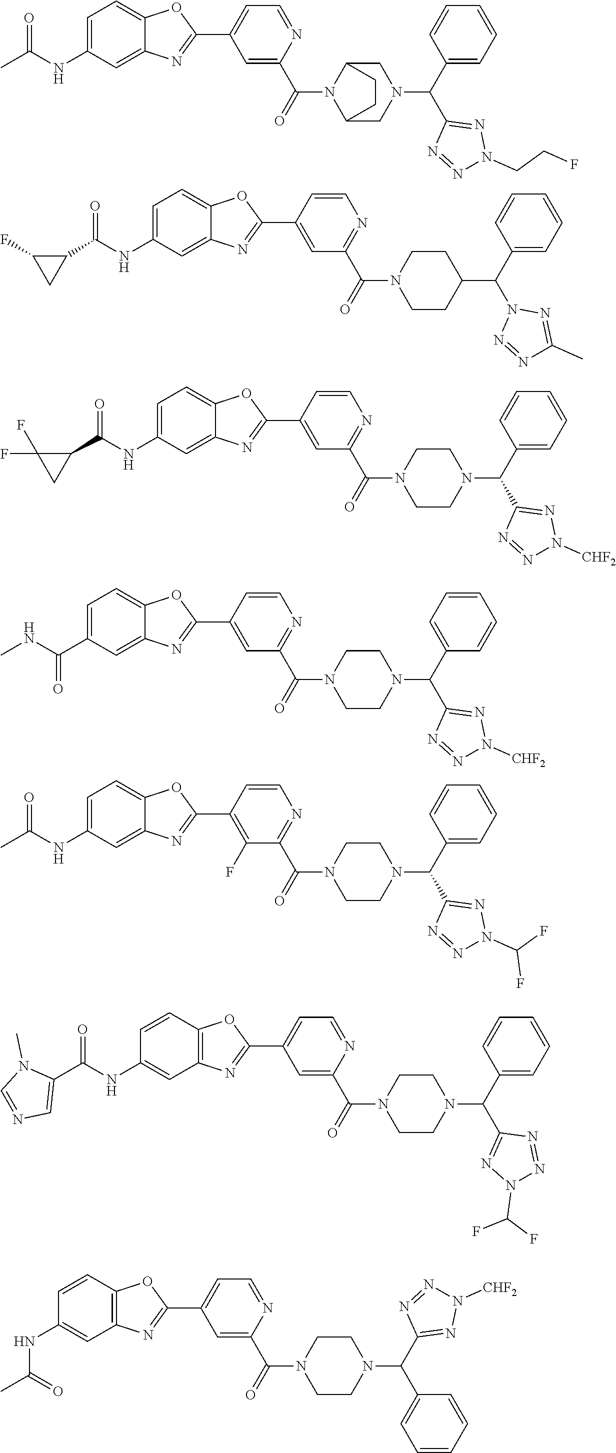

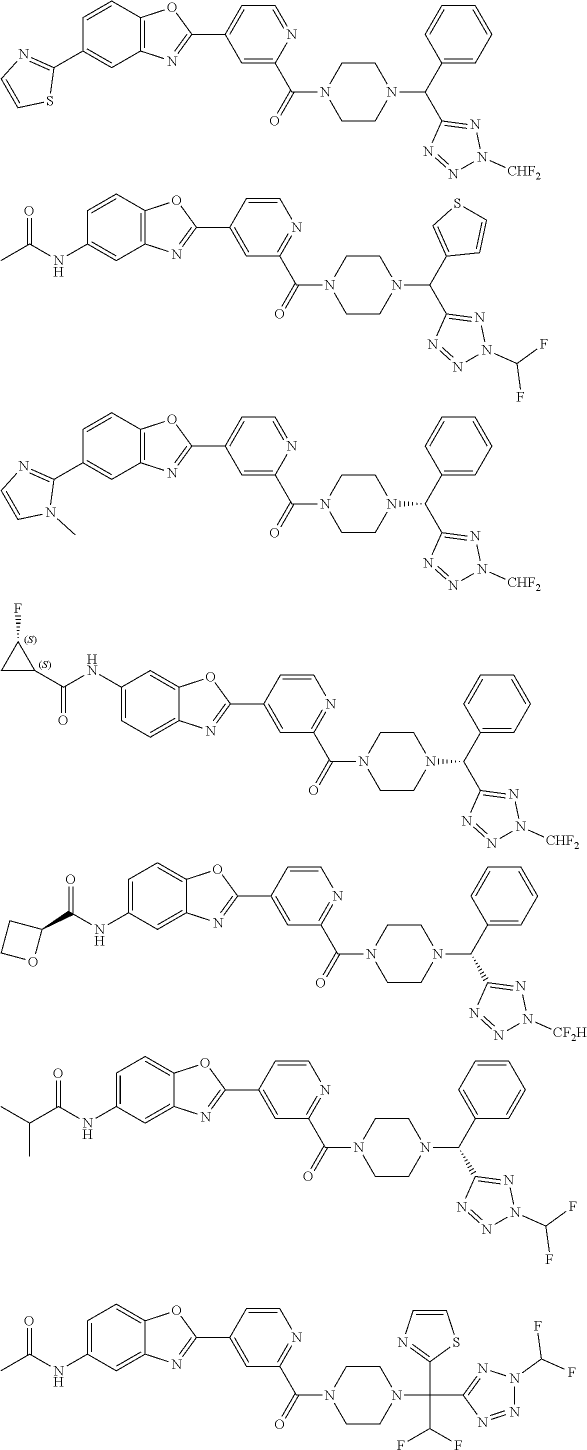

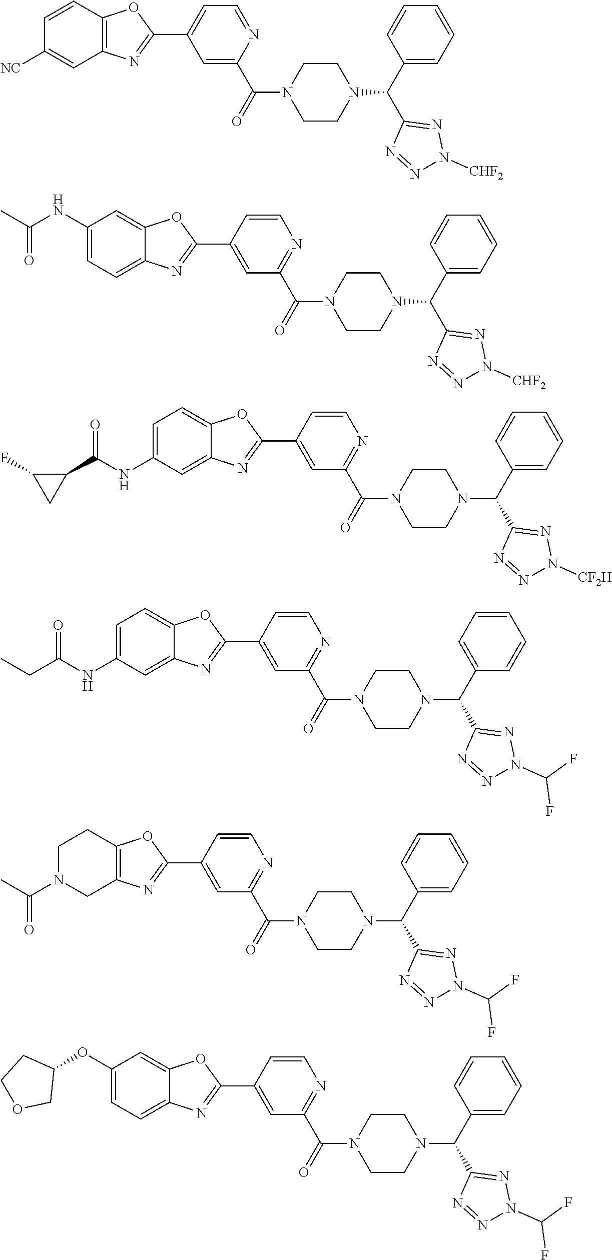

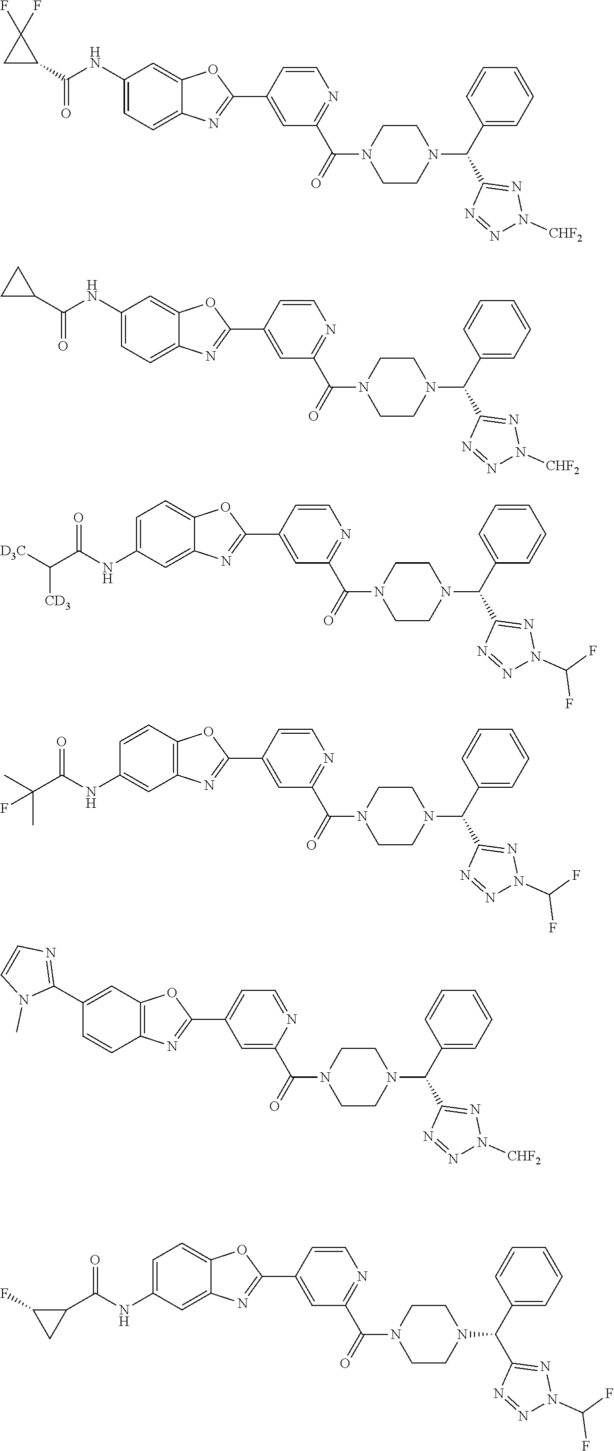

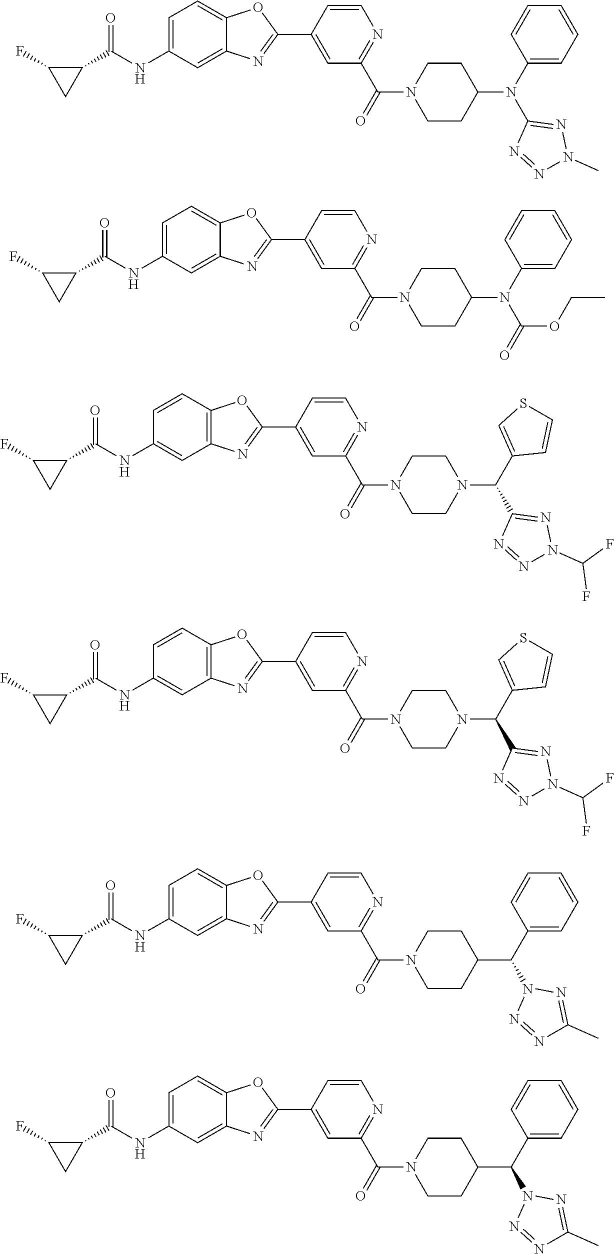

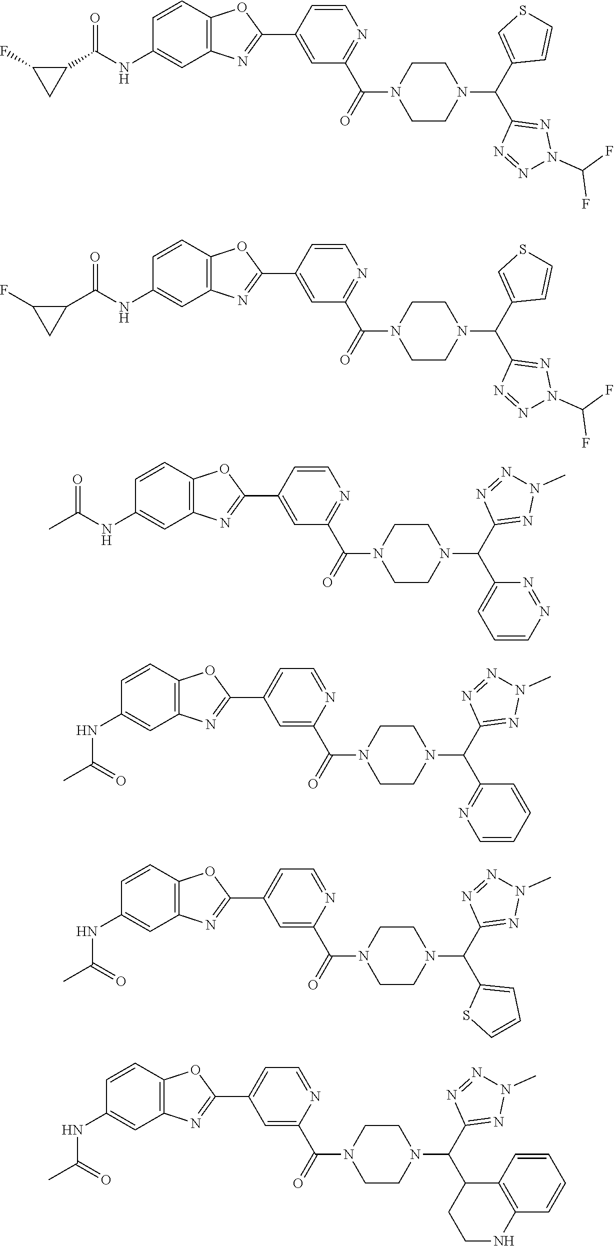

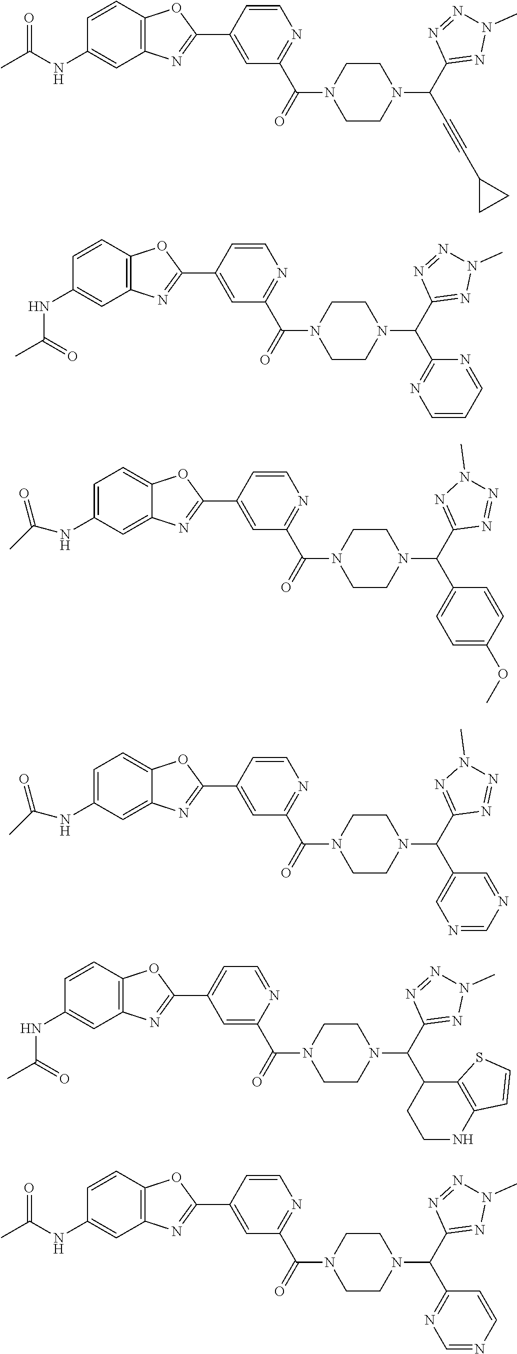

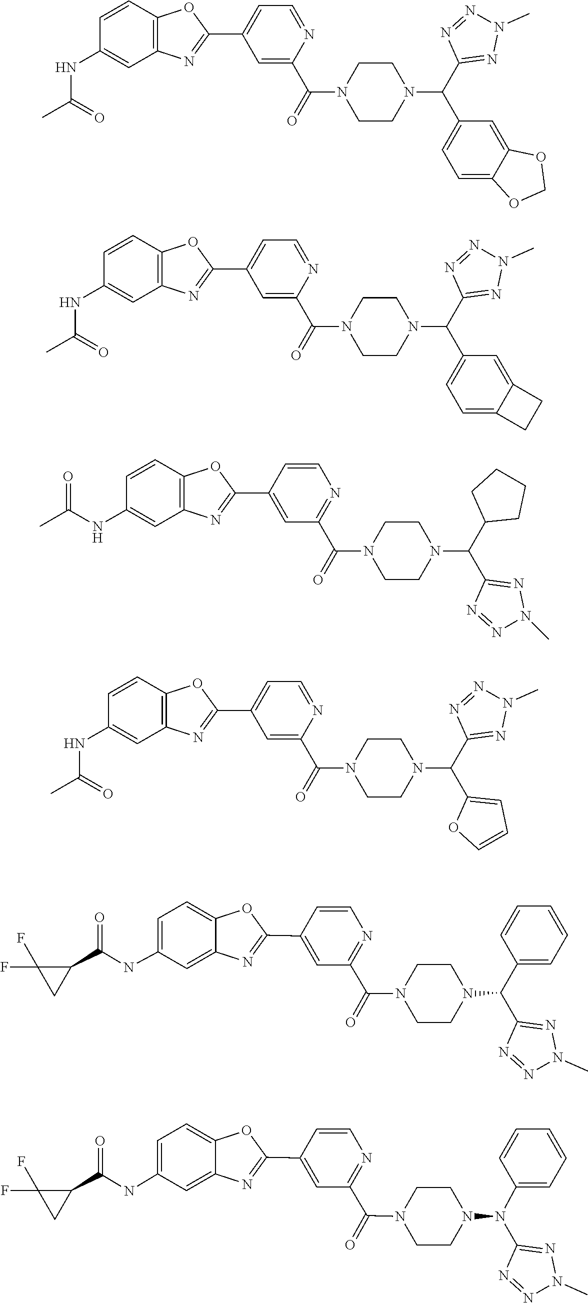

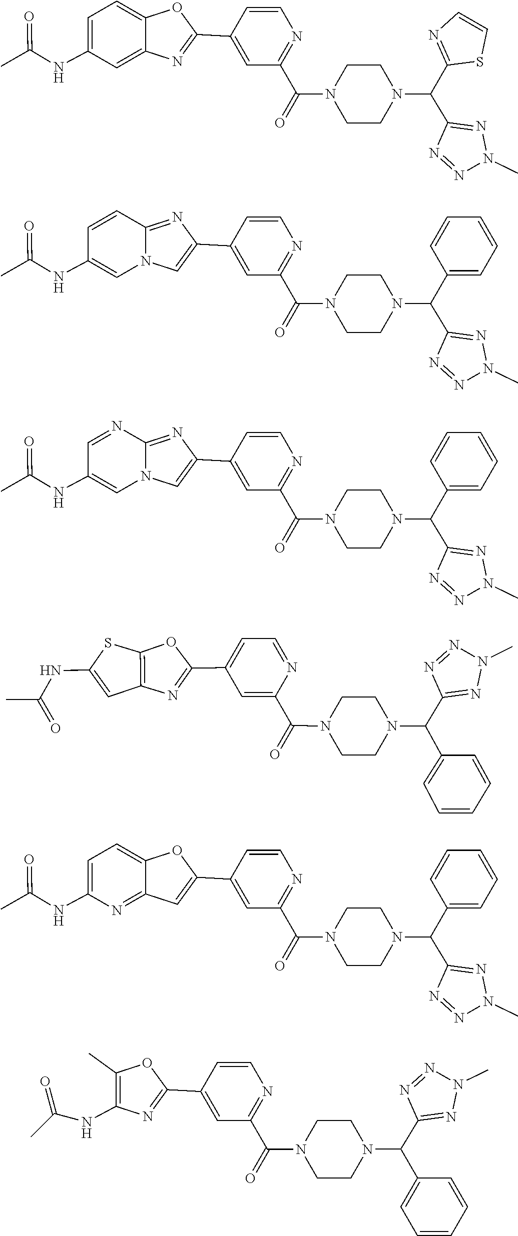

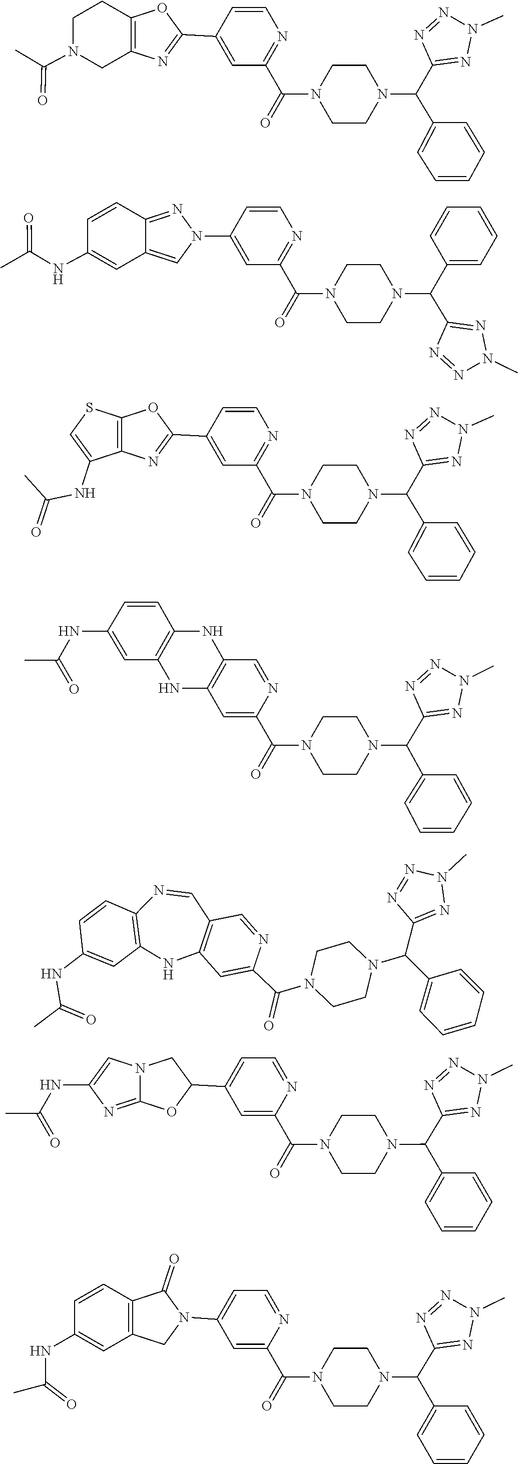

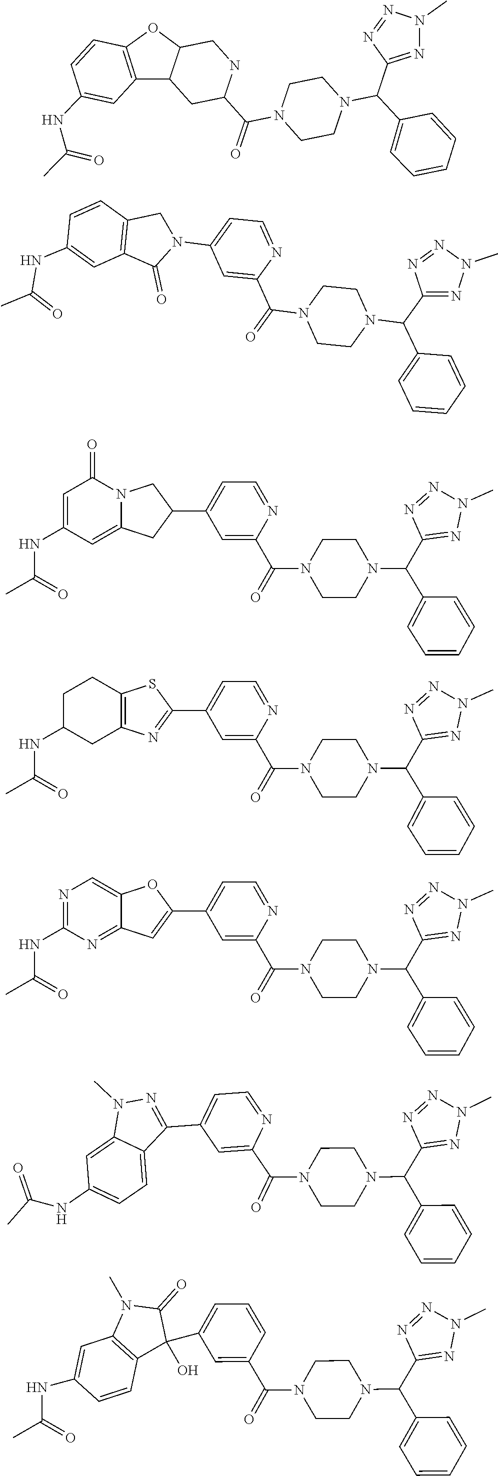

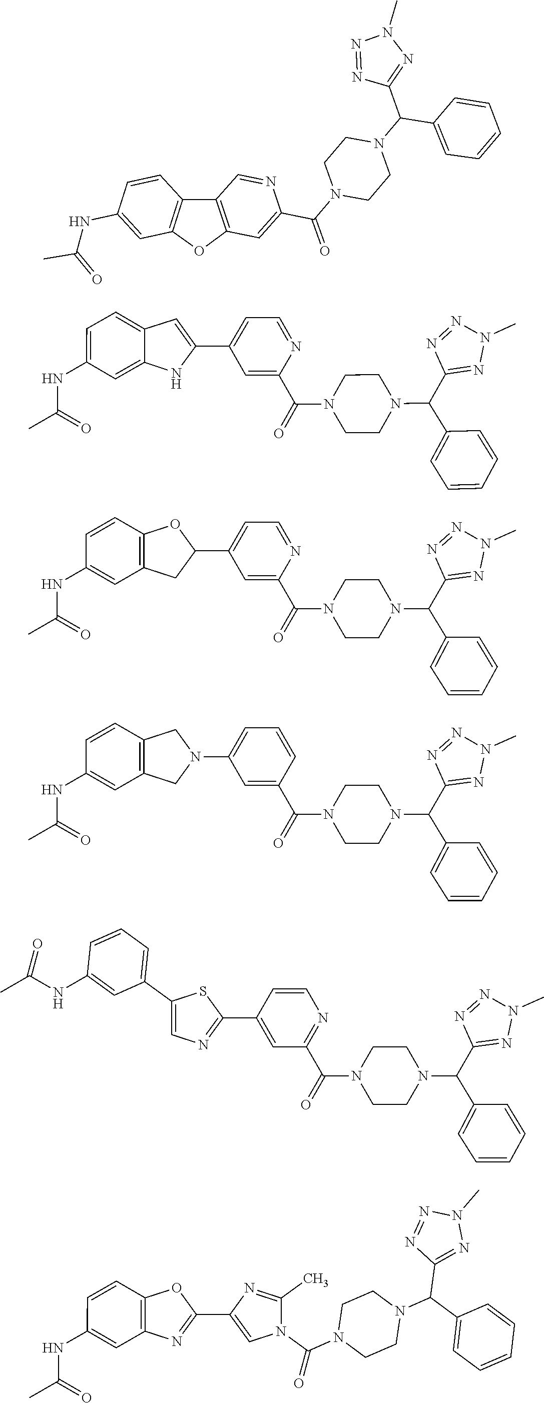

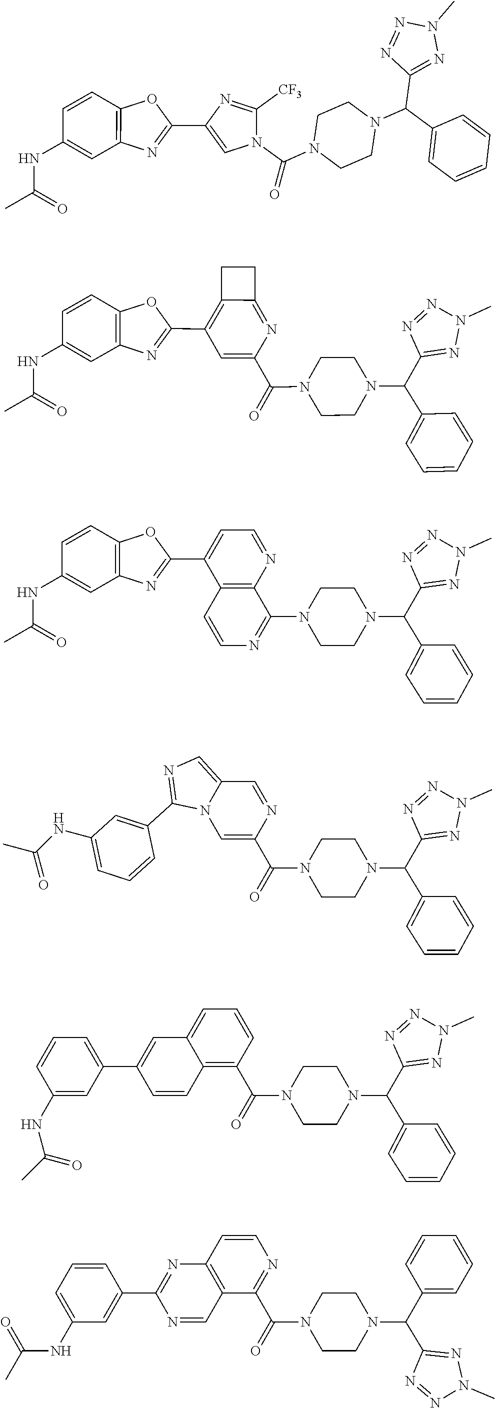

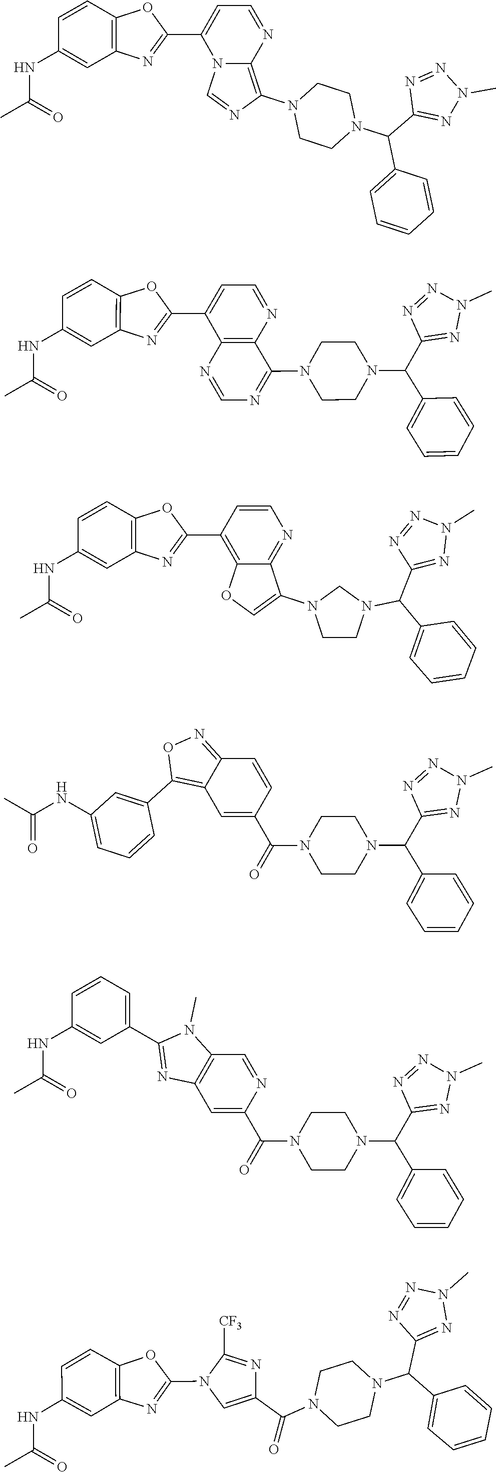

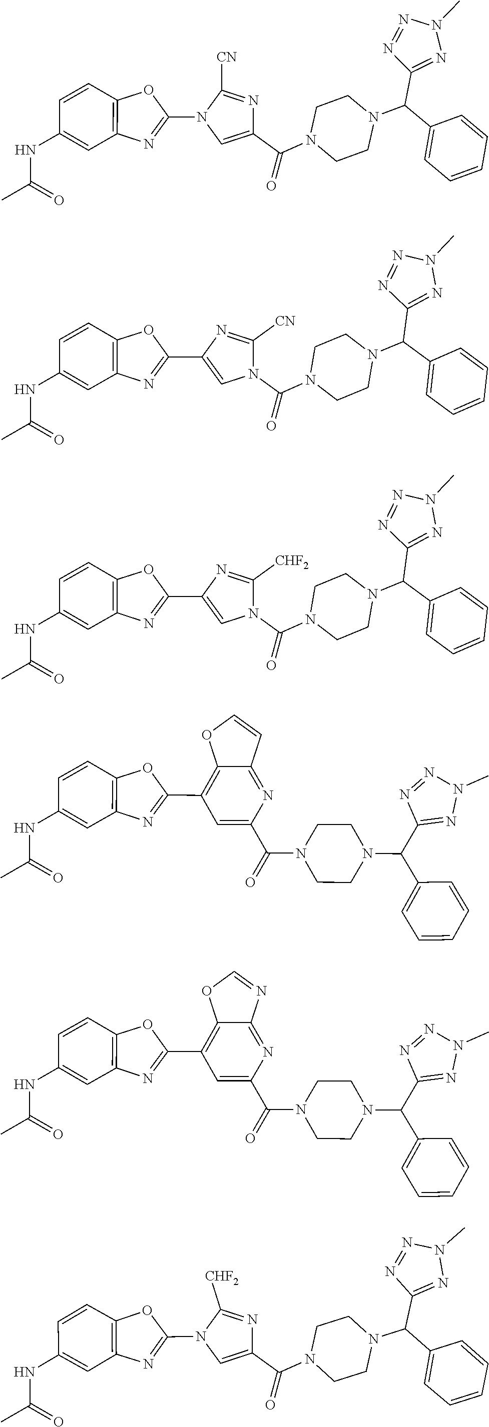

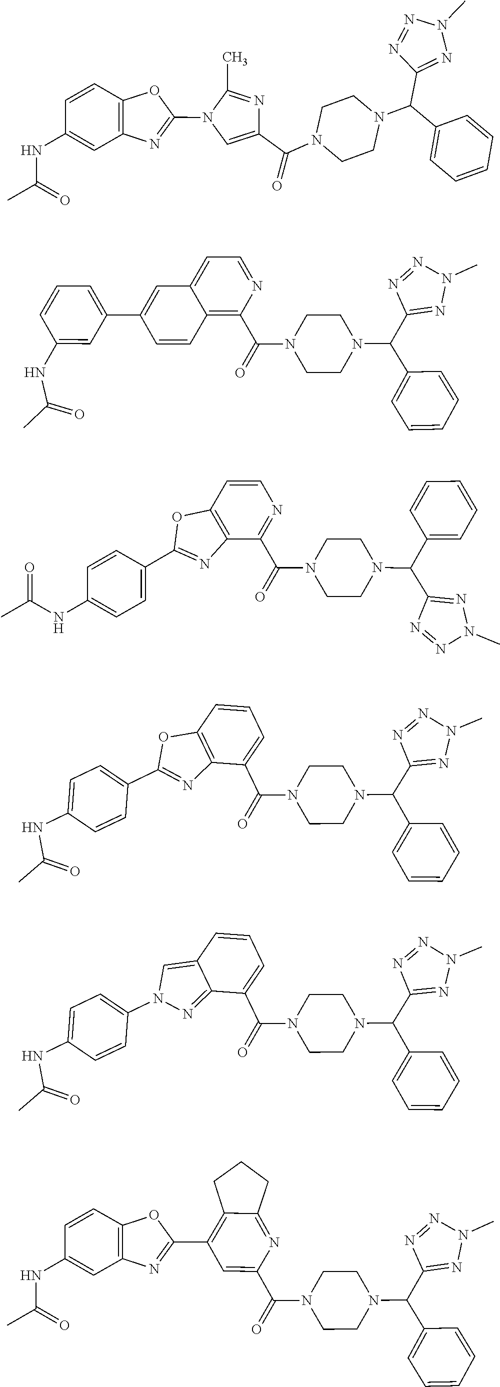

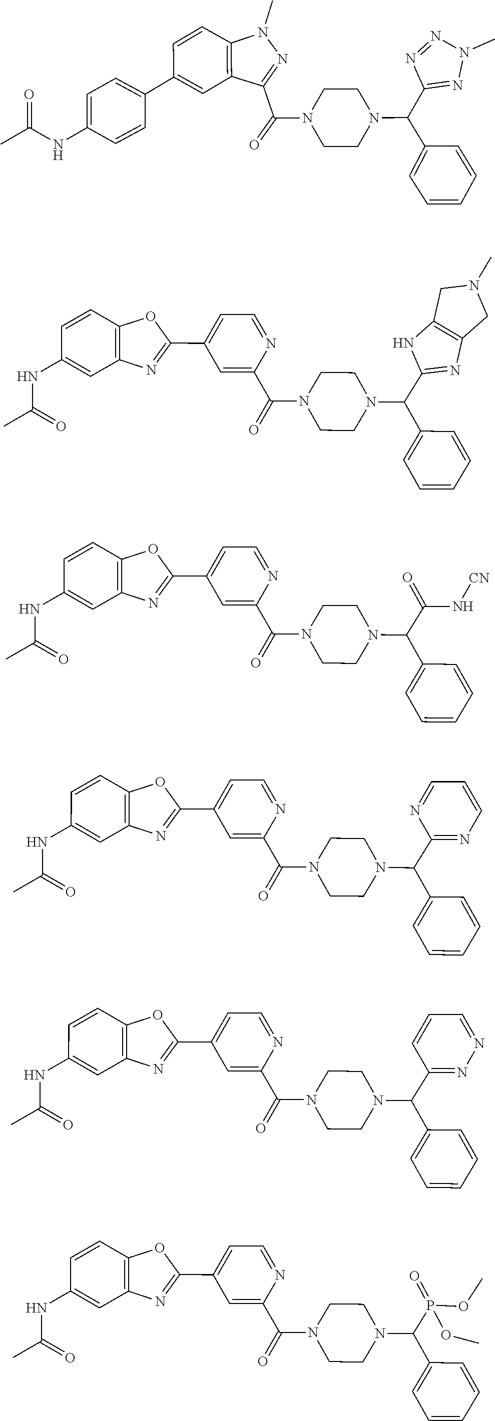

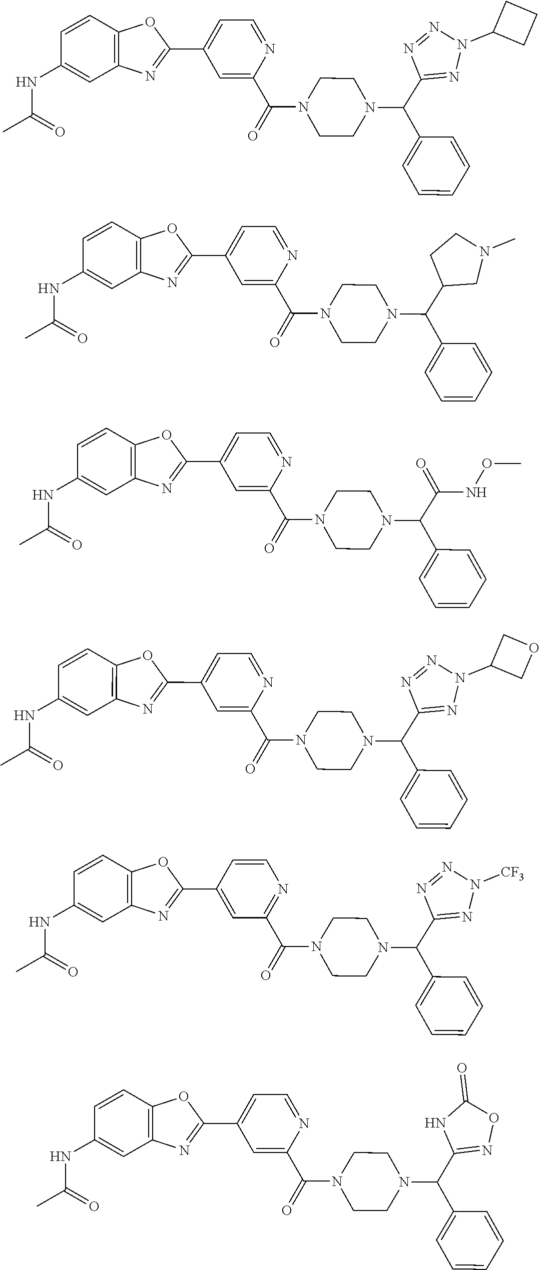

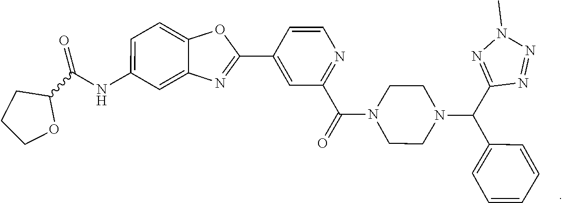 [SMILES: Cn1nnc(C(c2ccccc2)N2CCN(C(=O)c3cc(-c4nc5cc(NC(=O)C6CCCO6)ccc5o4)ccn3)CC2)n1]

28. The compound, the isomer thereof or the pharmaceutically acceptable salt thereof according to claim 27, wherein the compound is selected from:

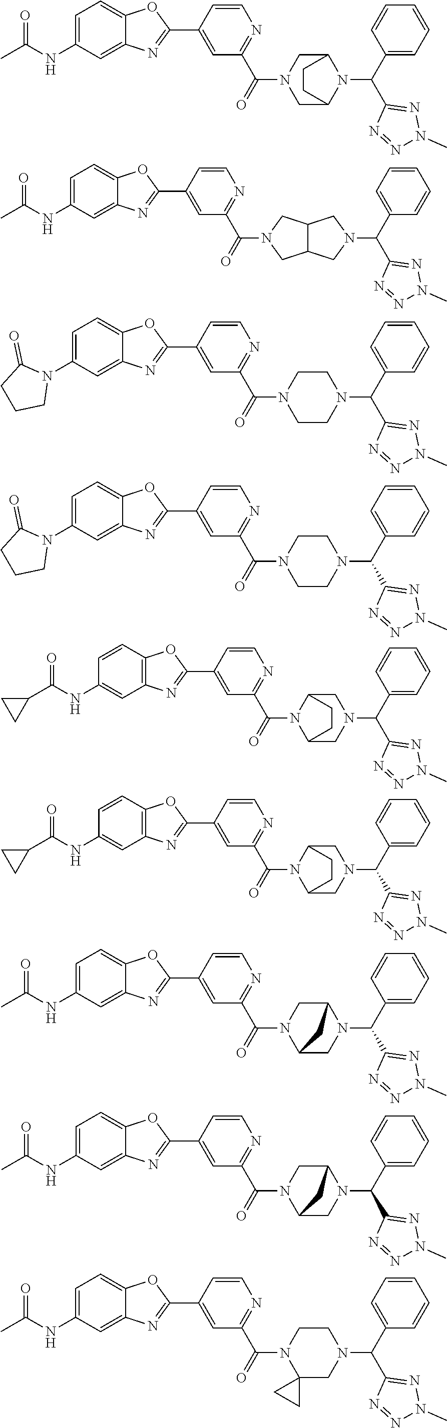

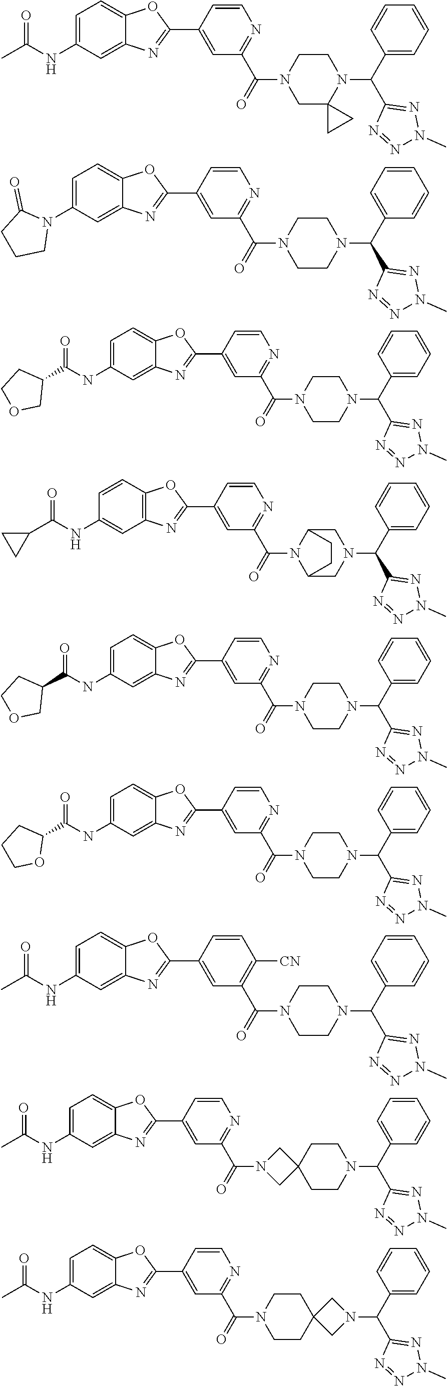

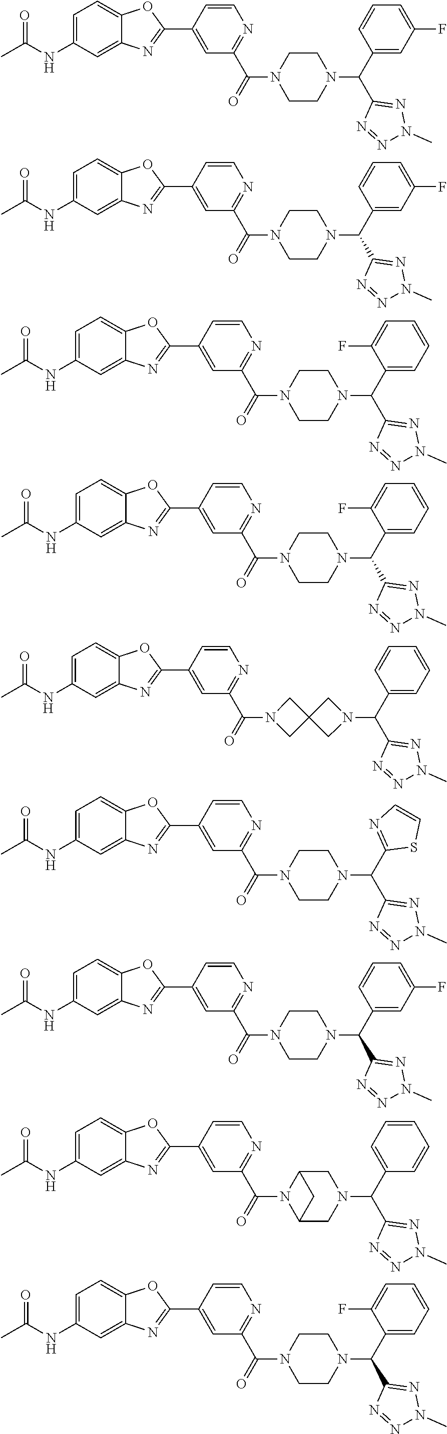

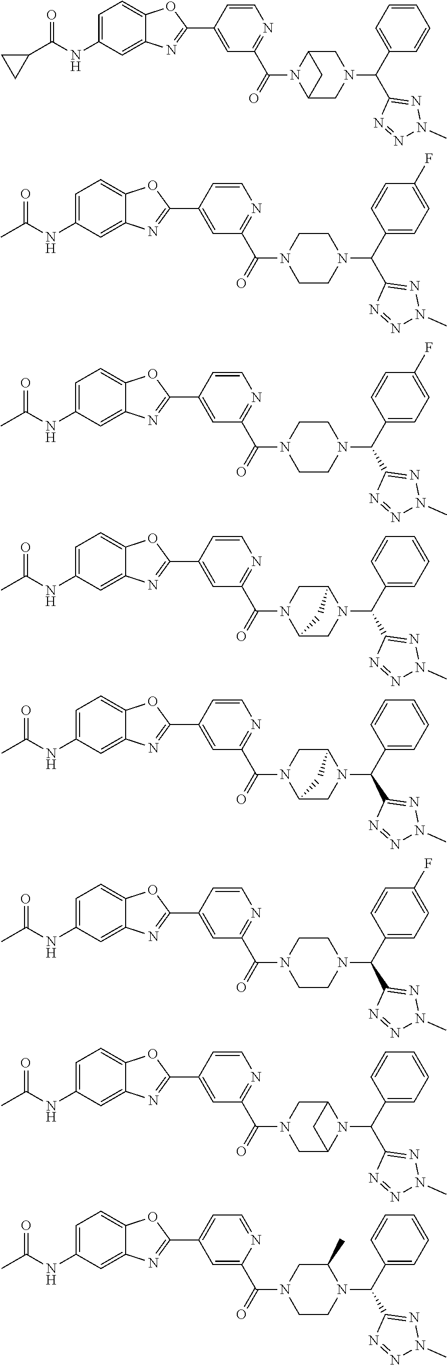

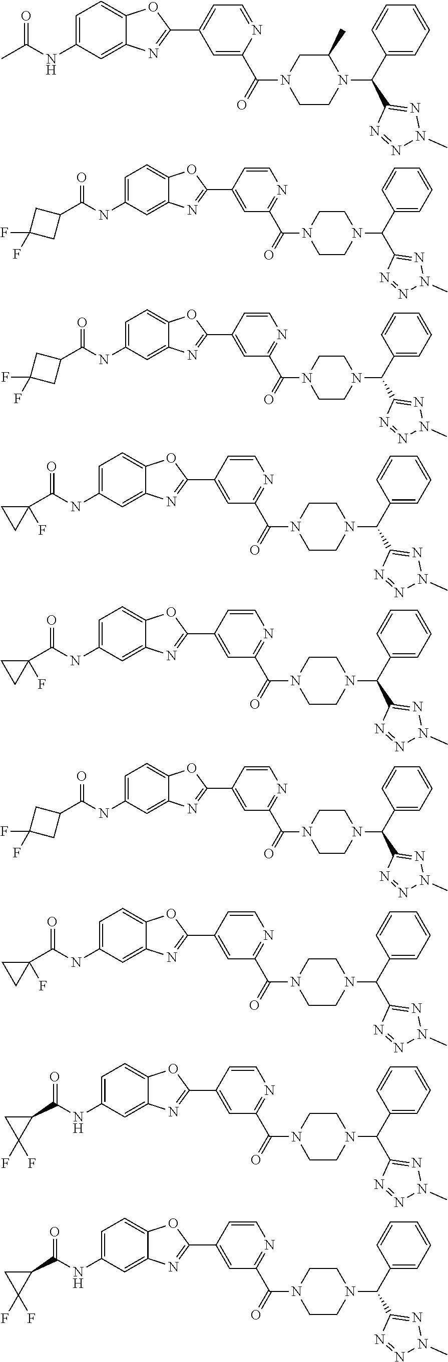

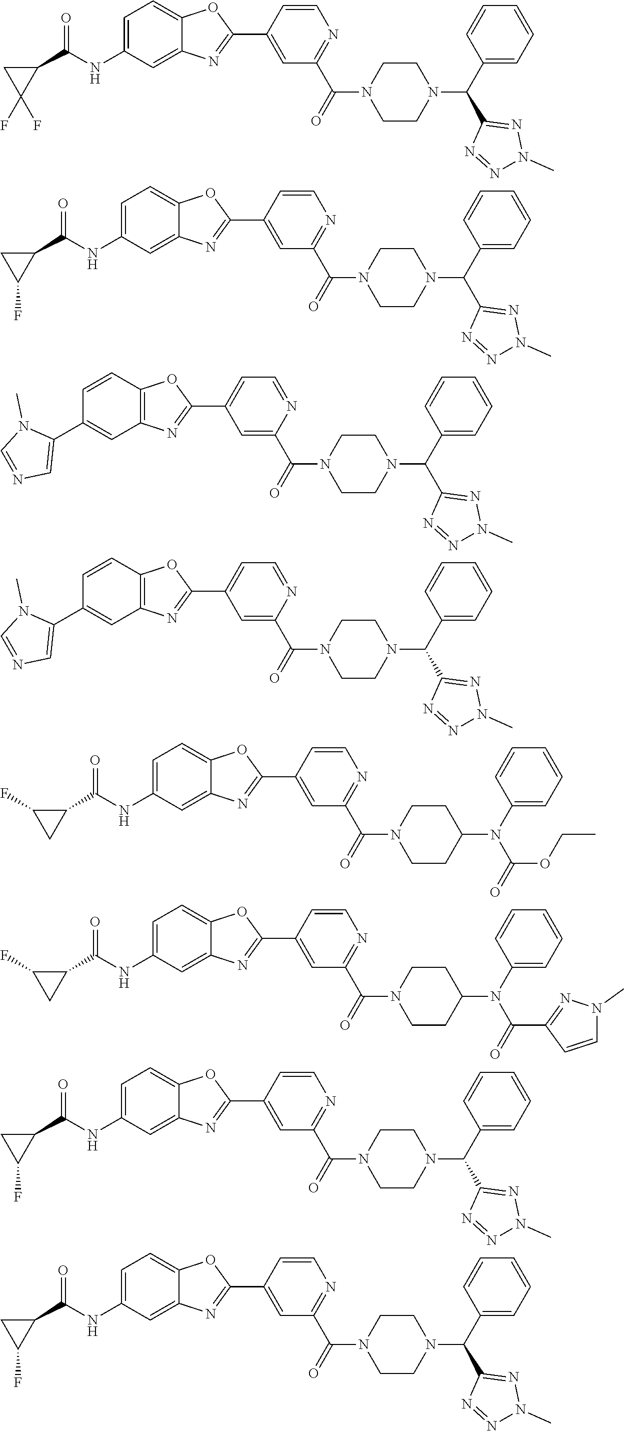

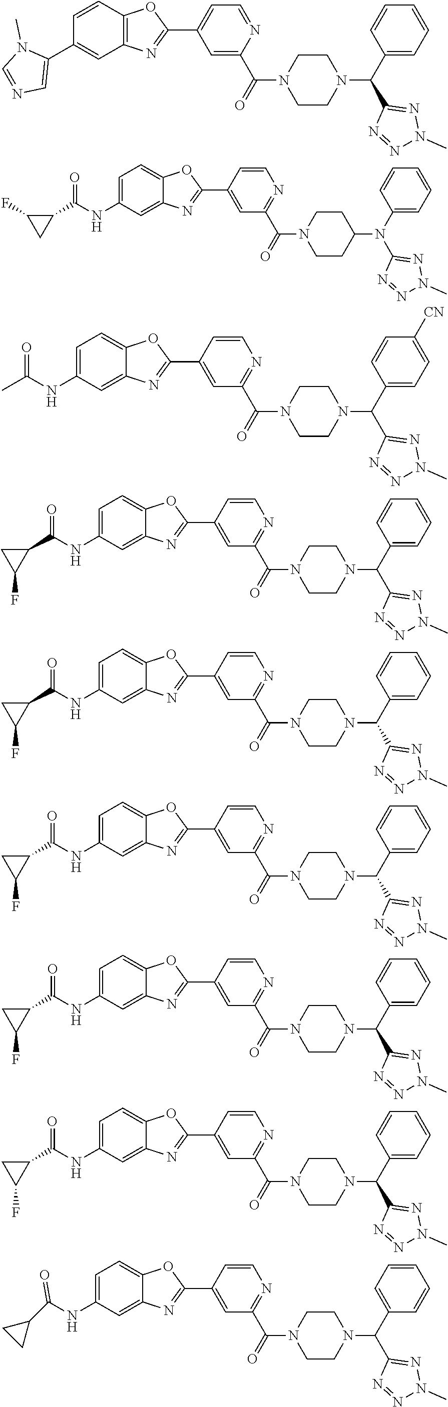

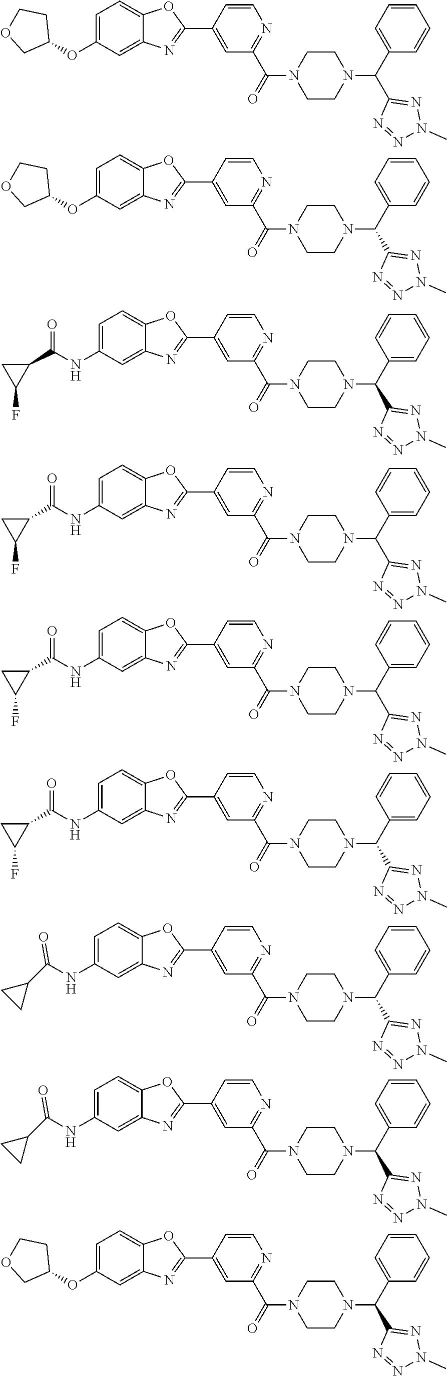

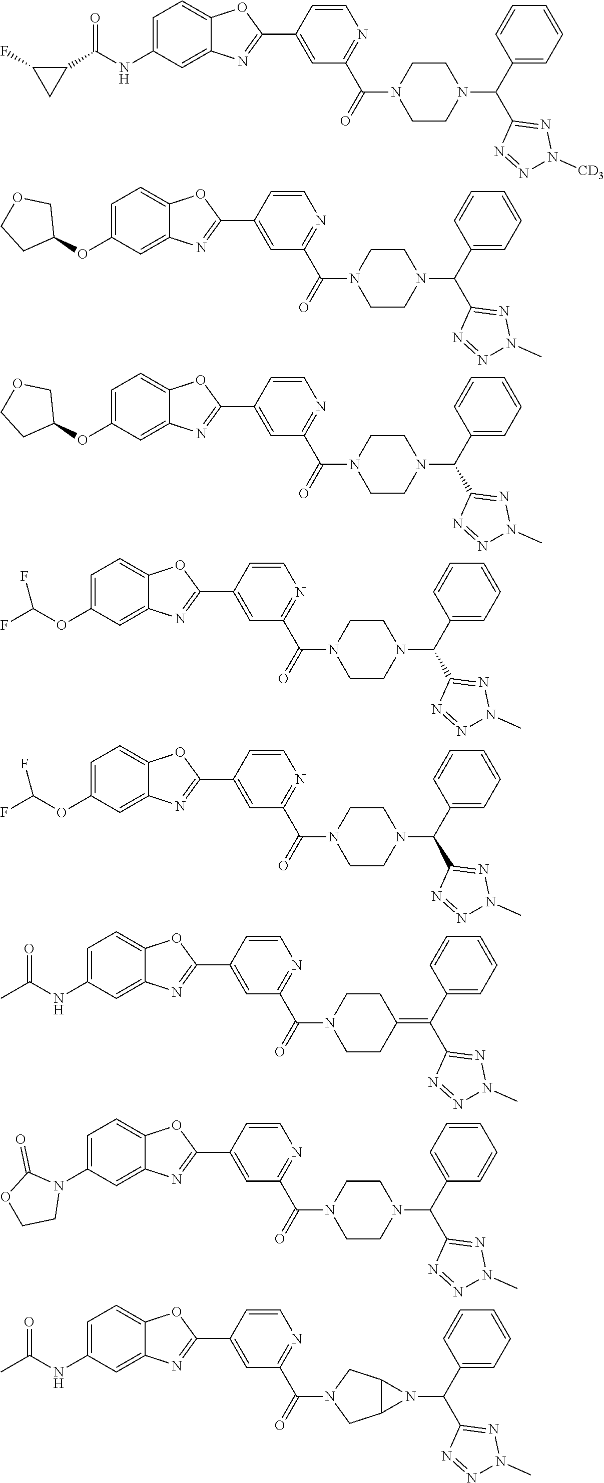

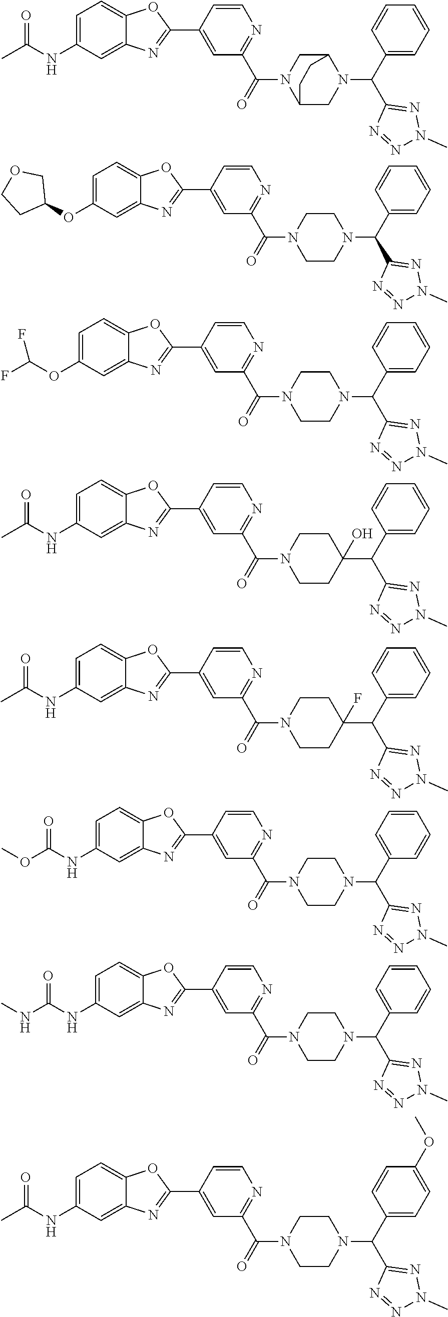

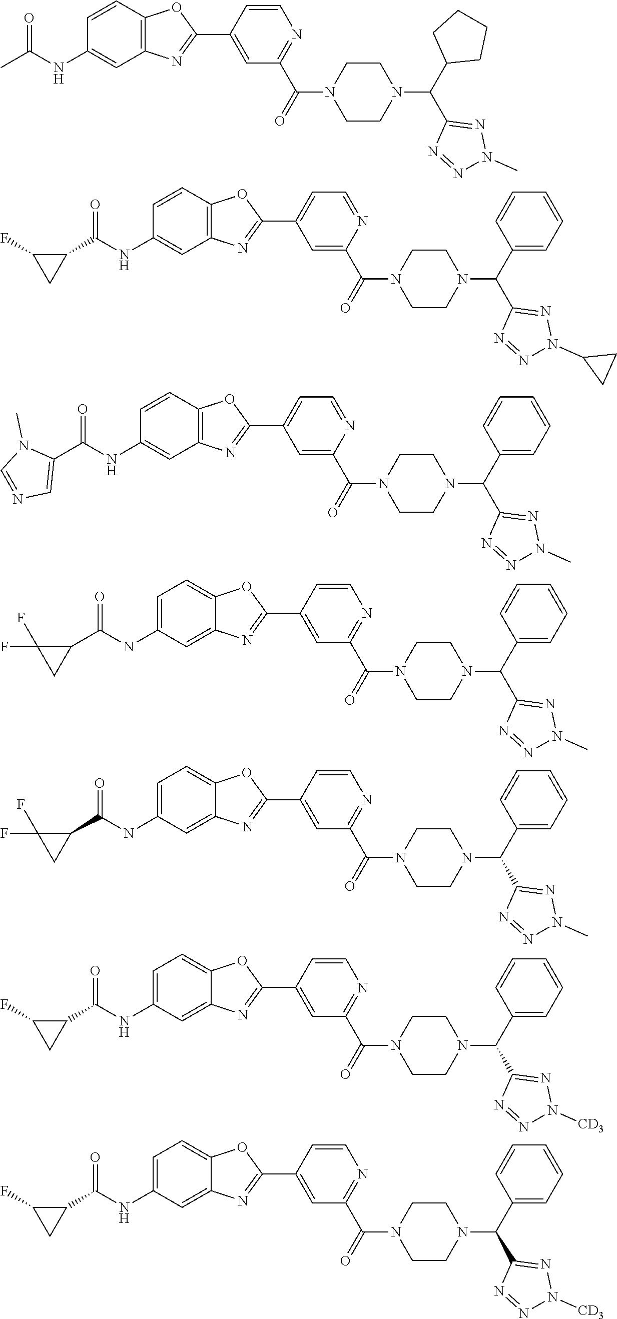

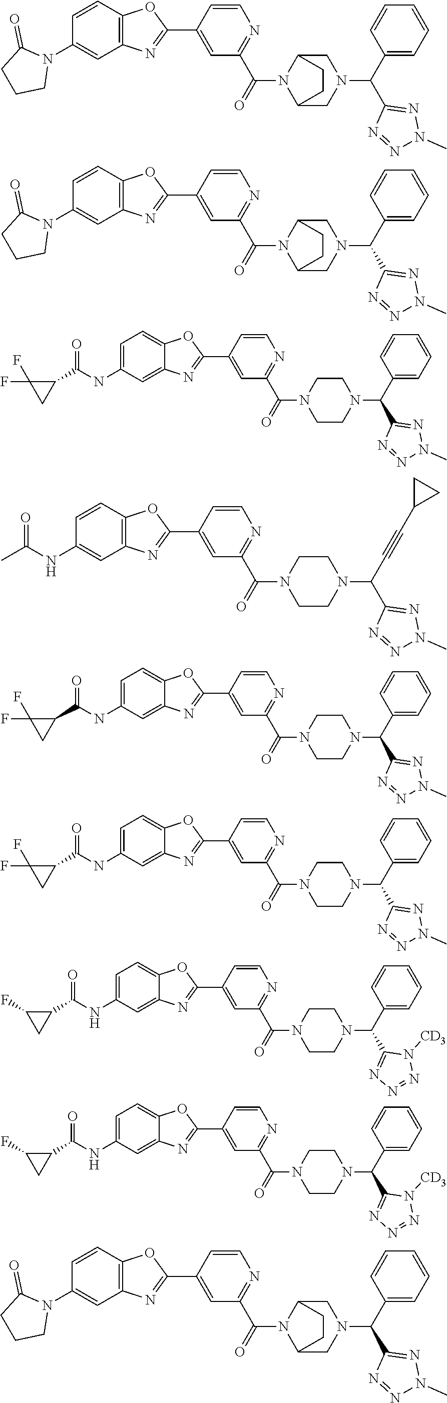

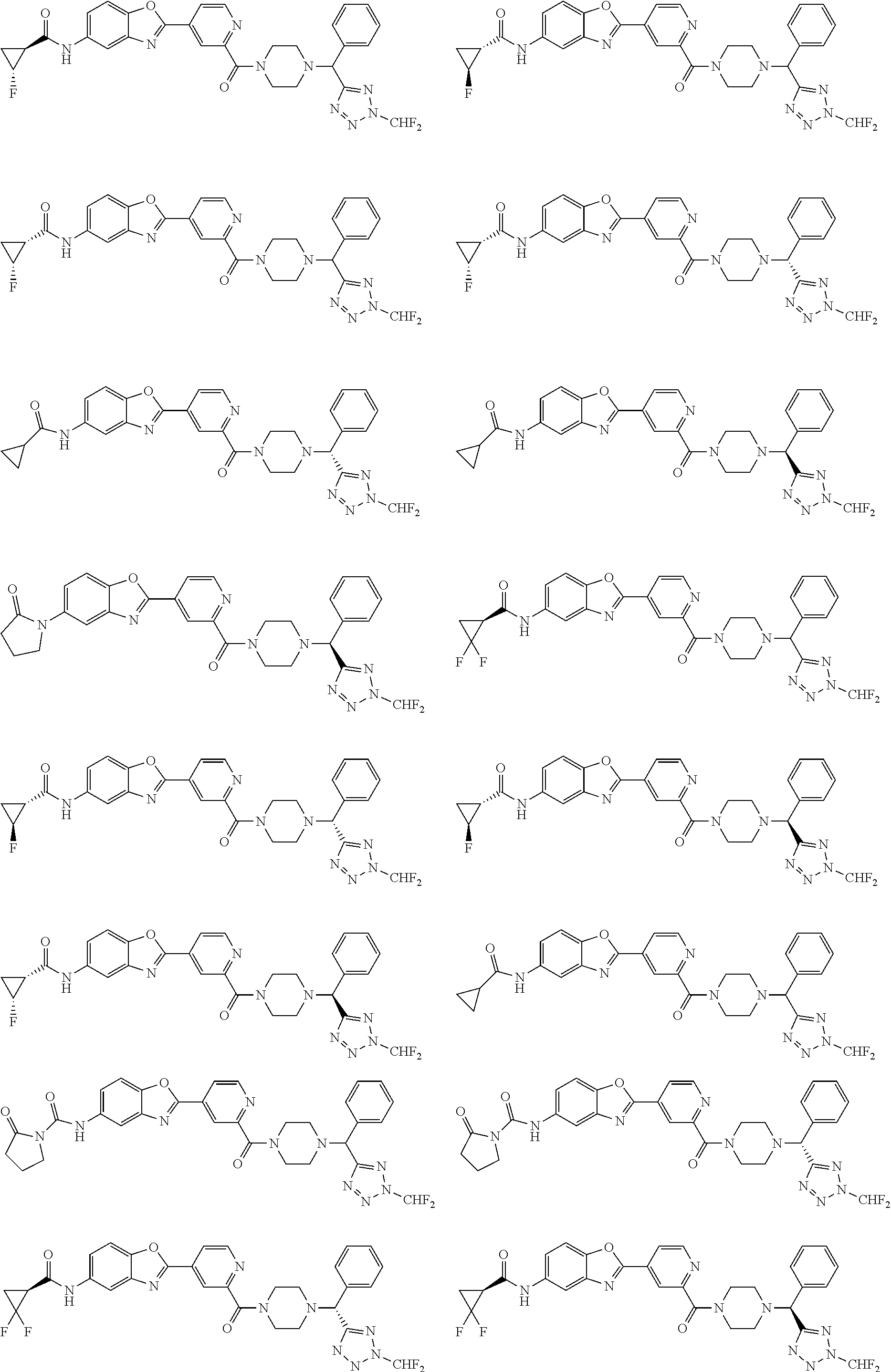

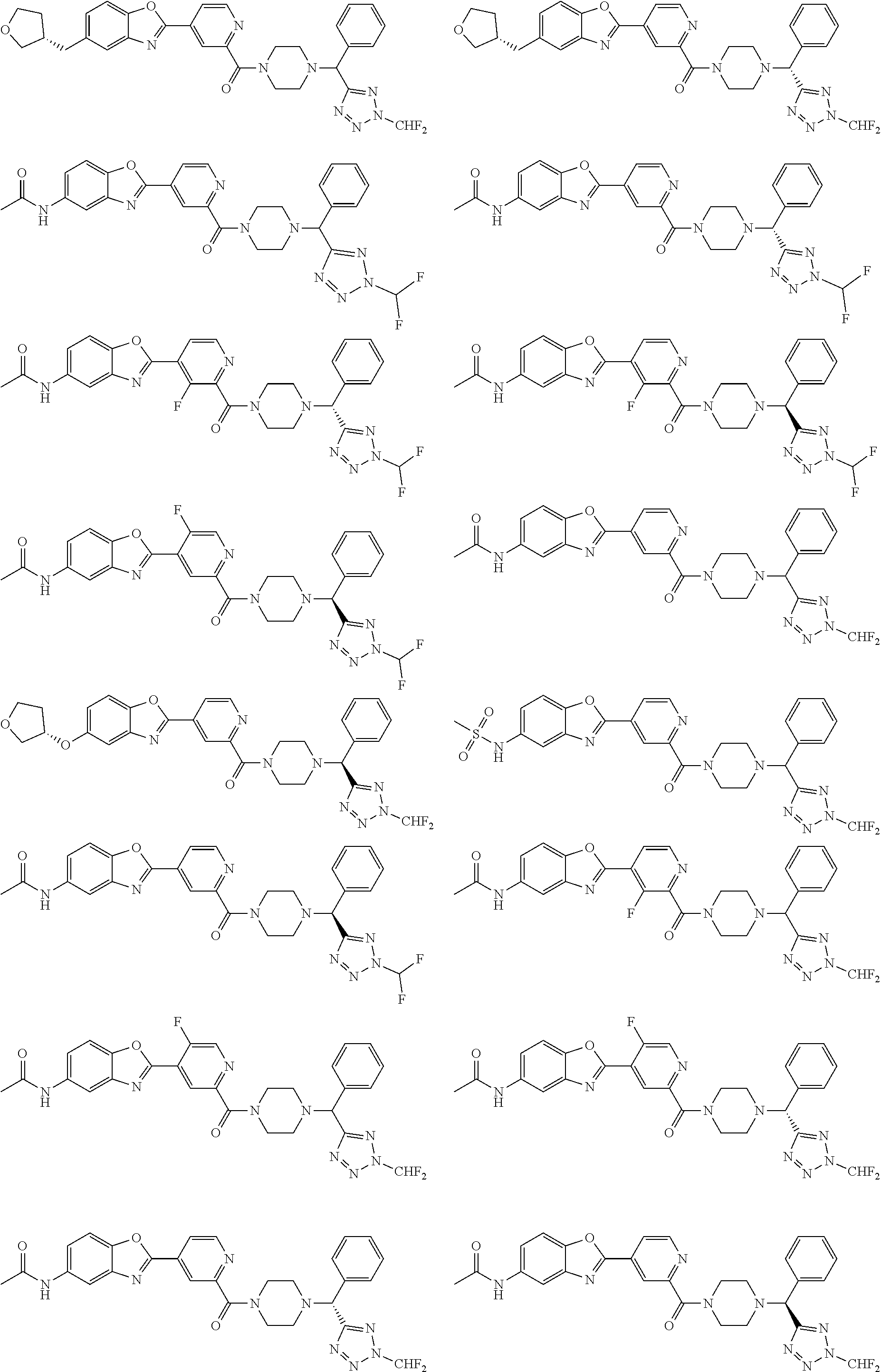

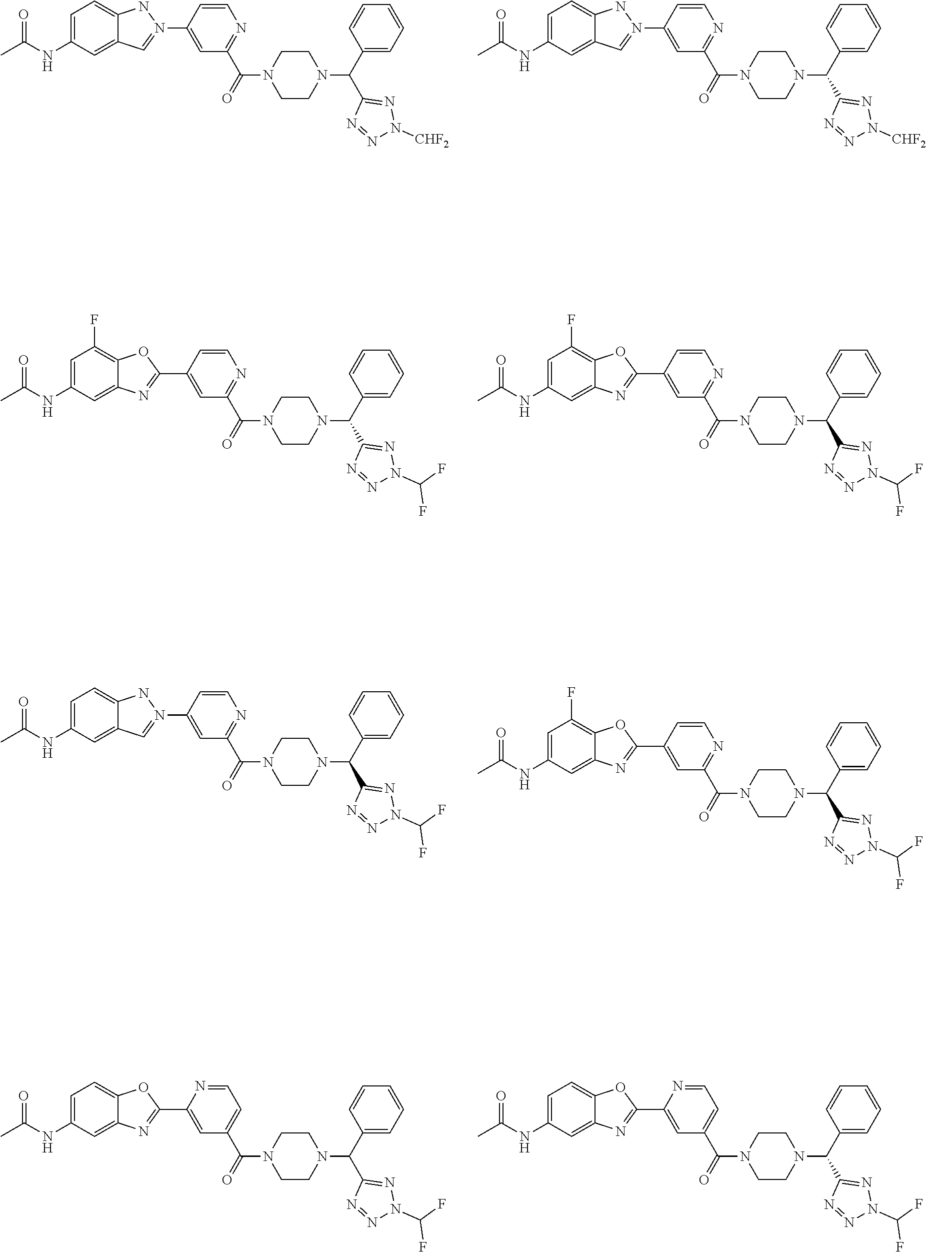

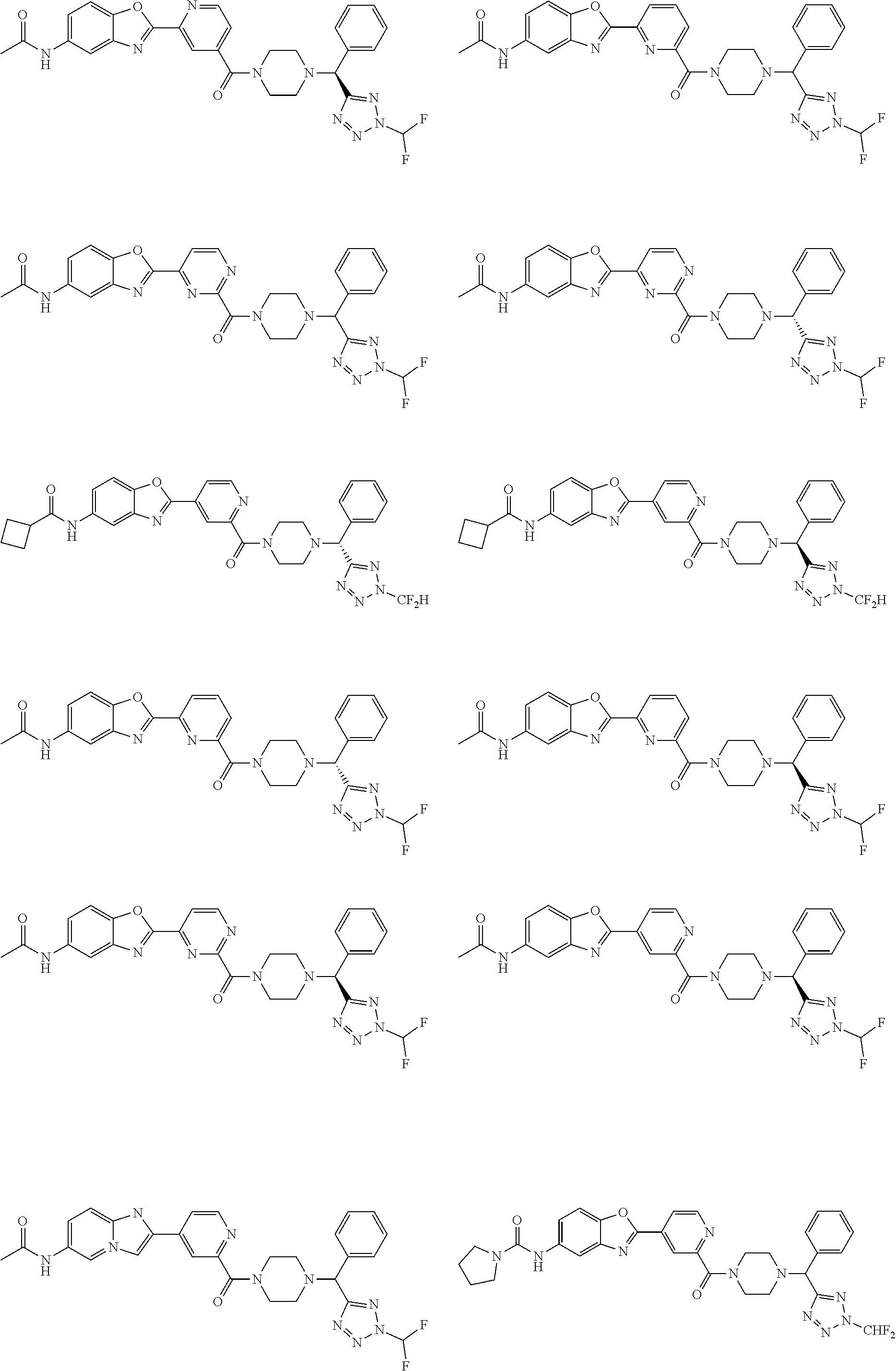

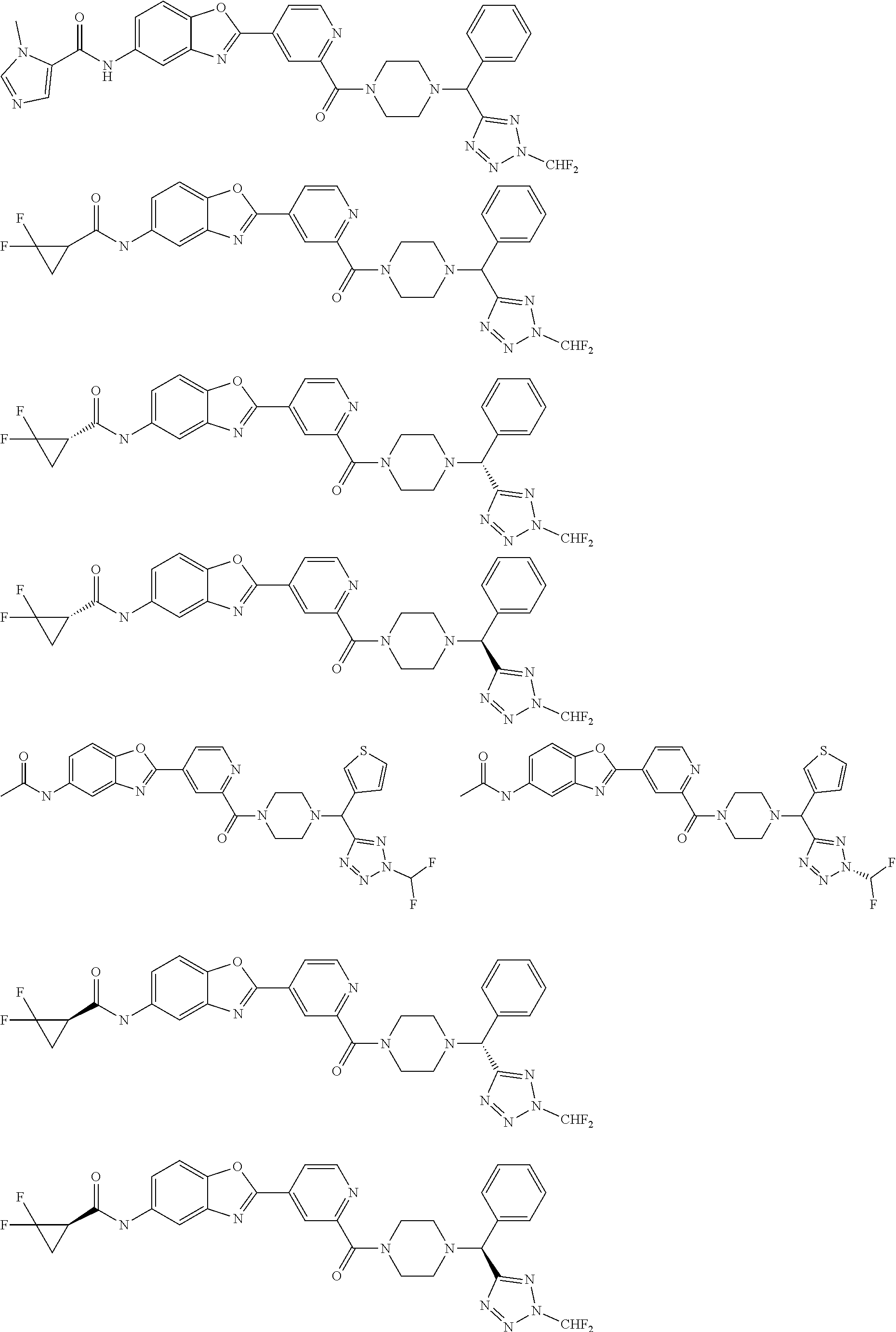

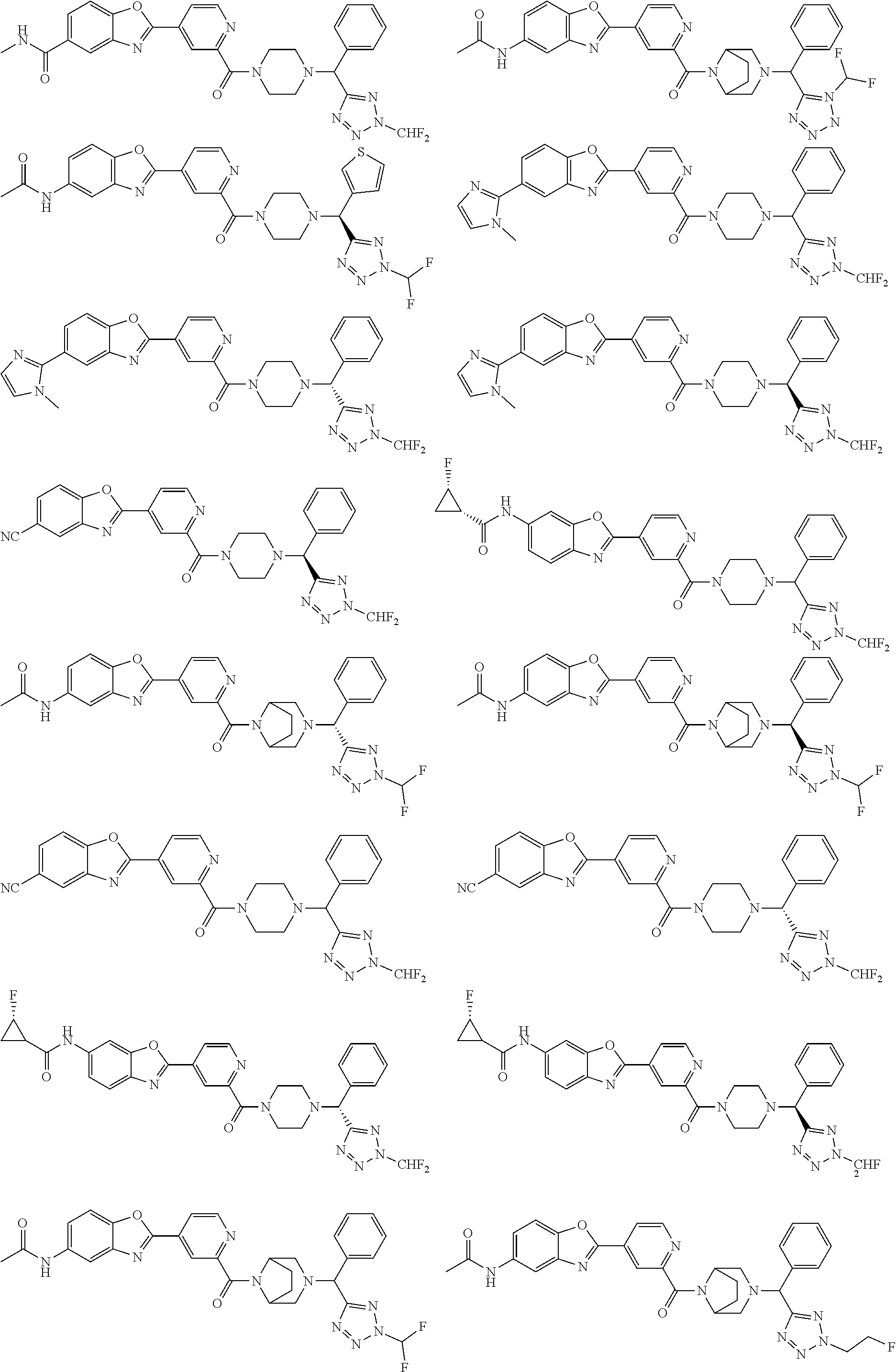

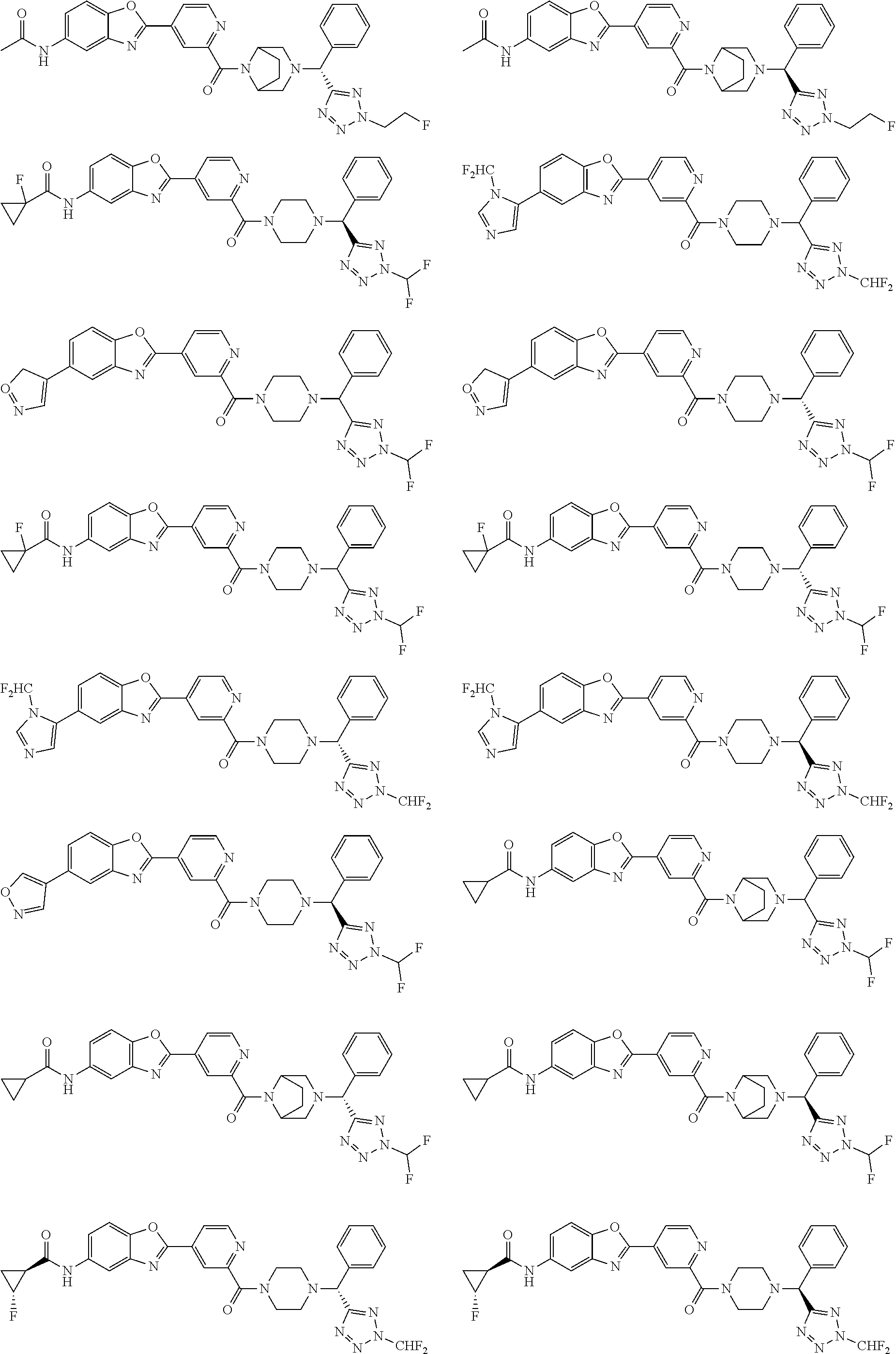

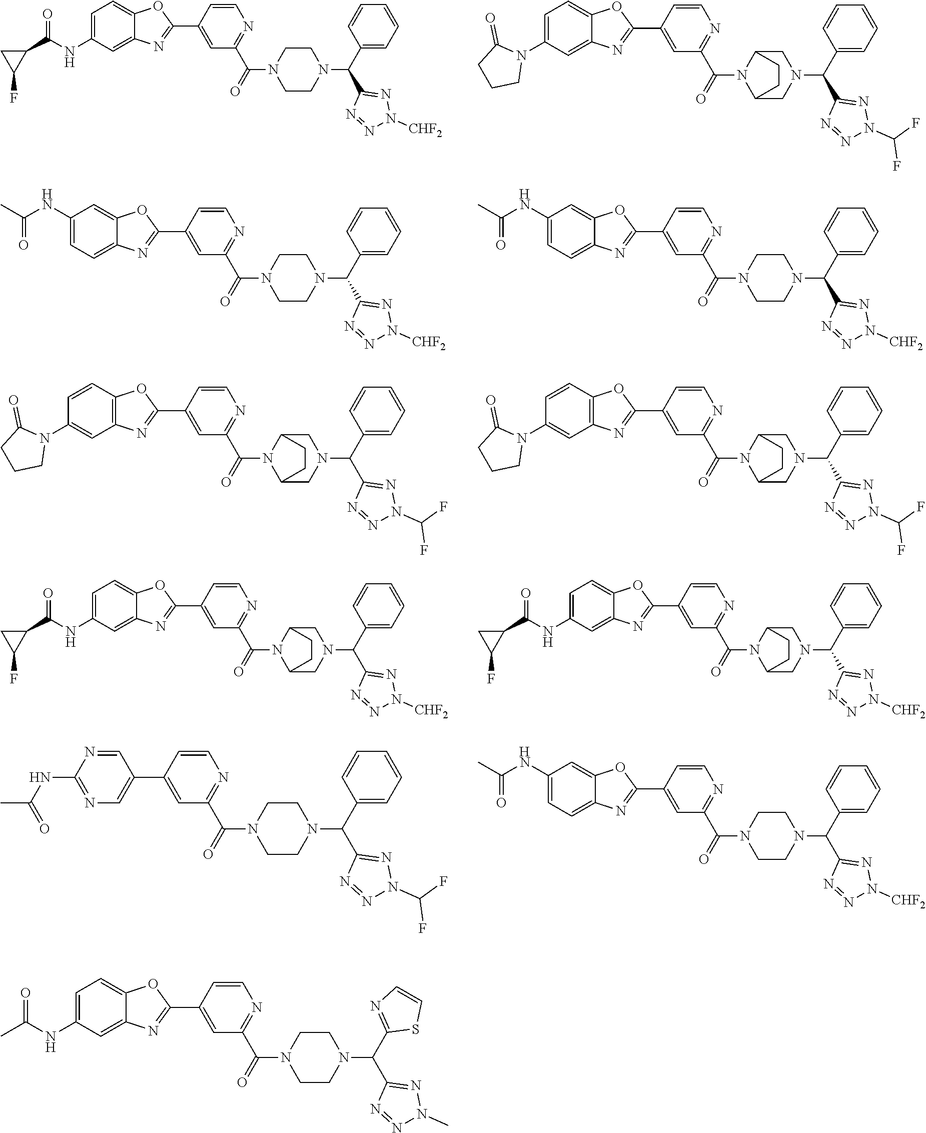

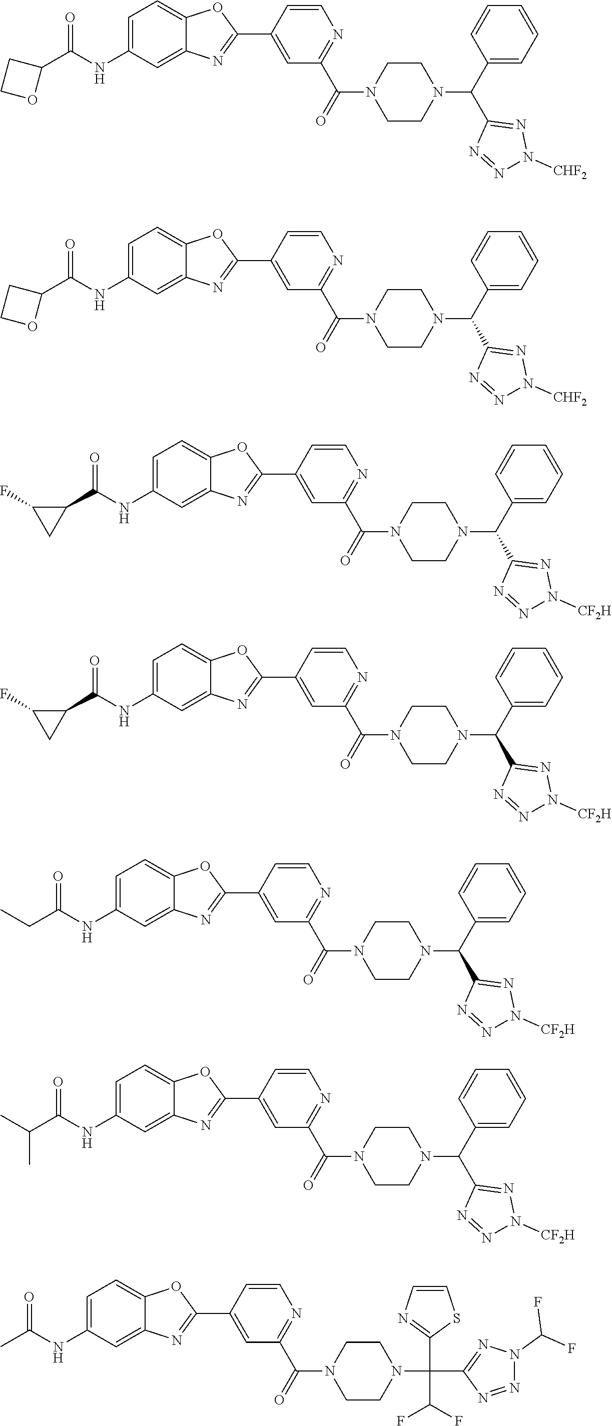

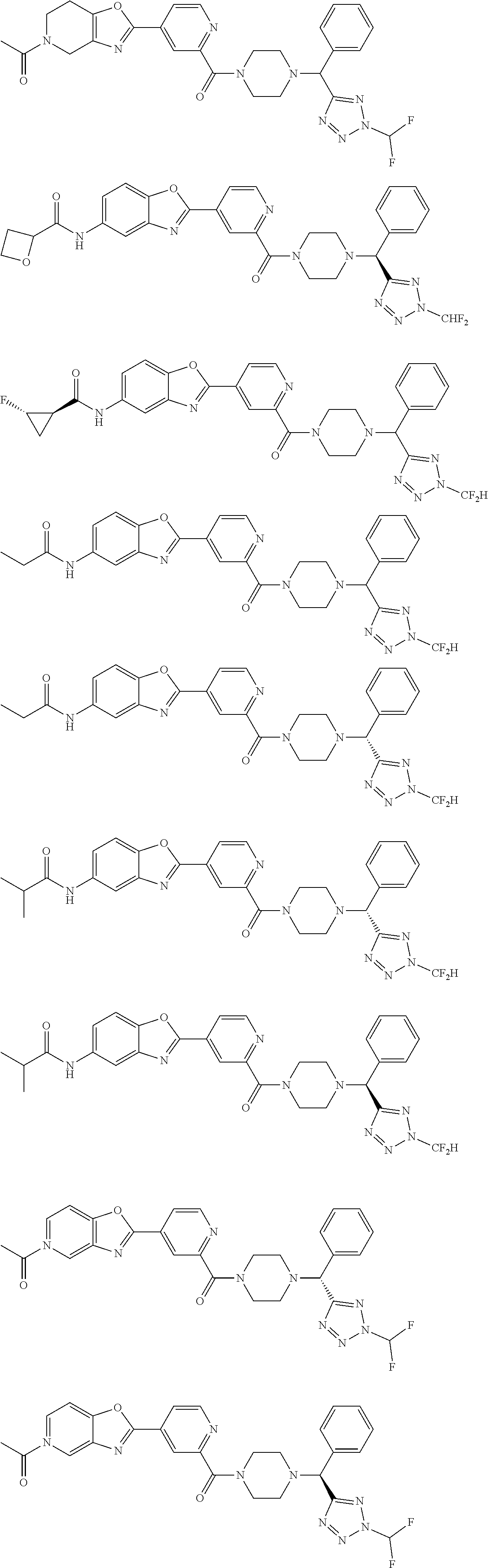

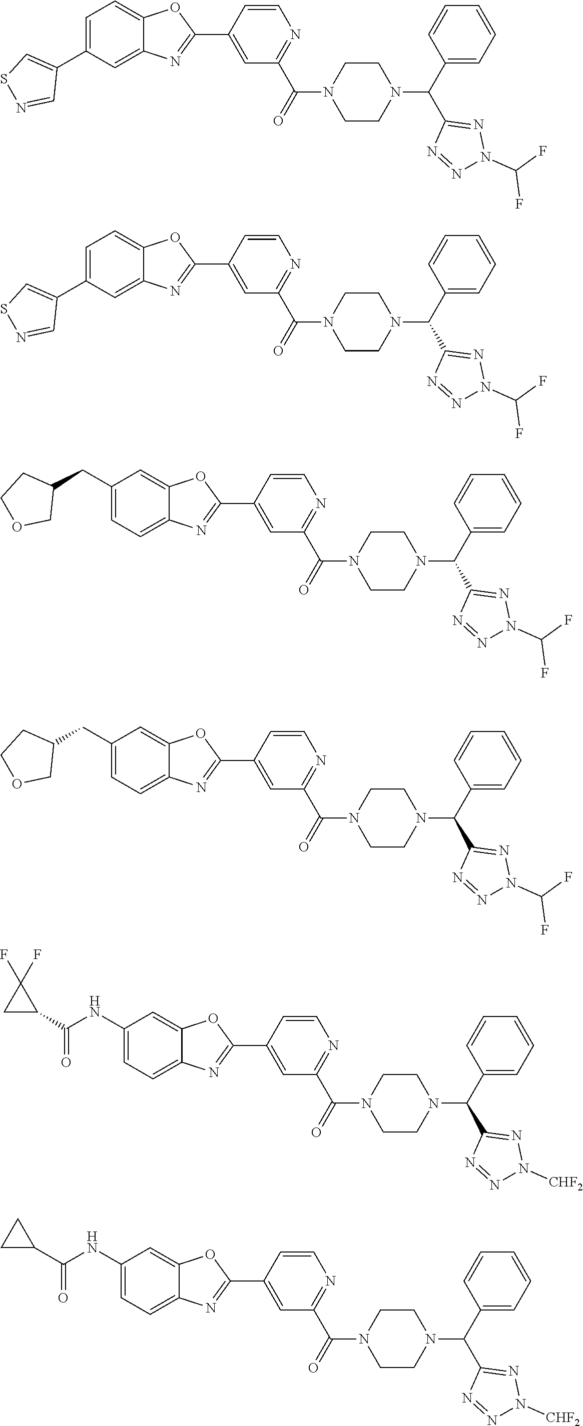

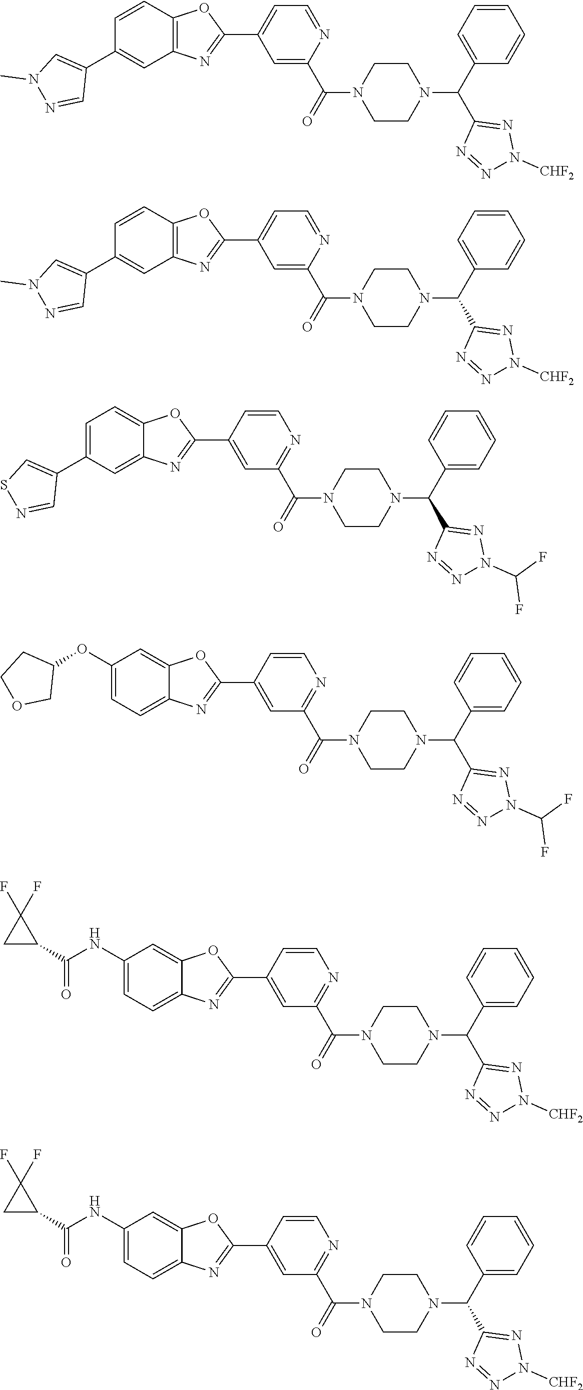

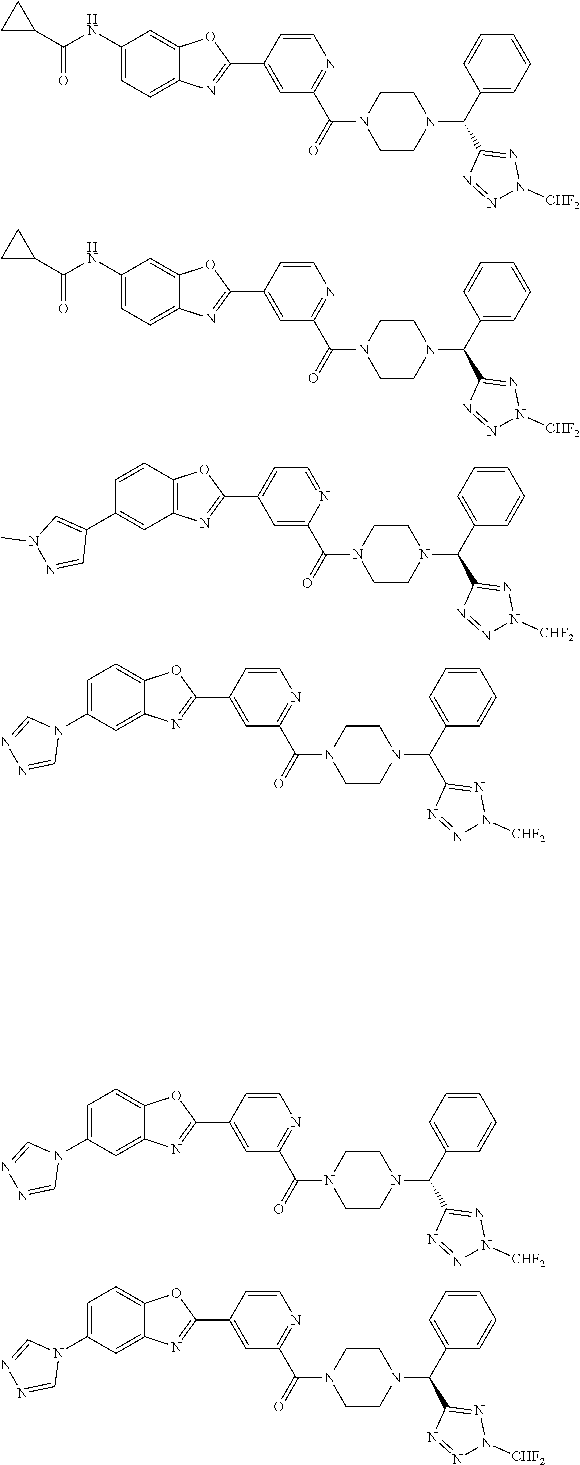

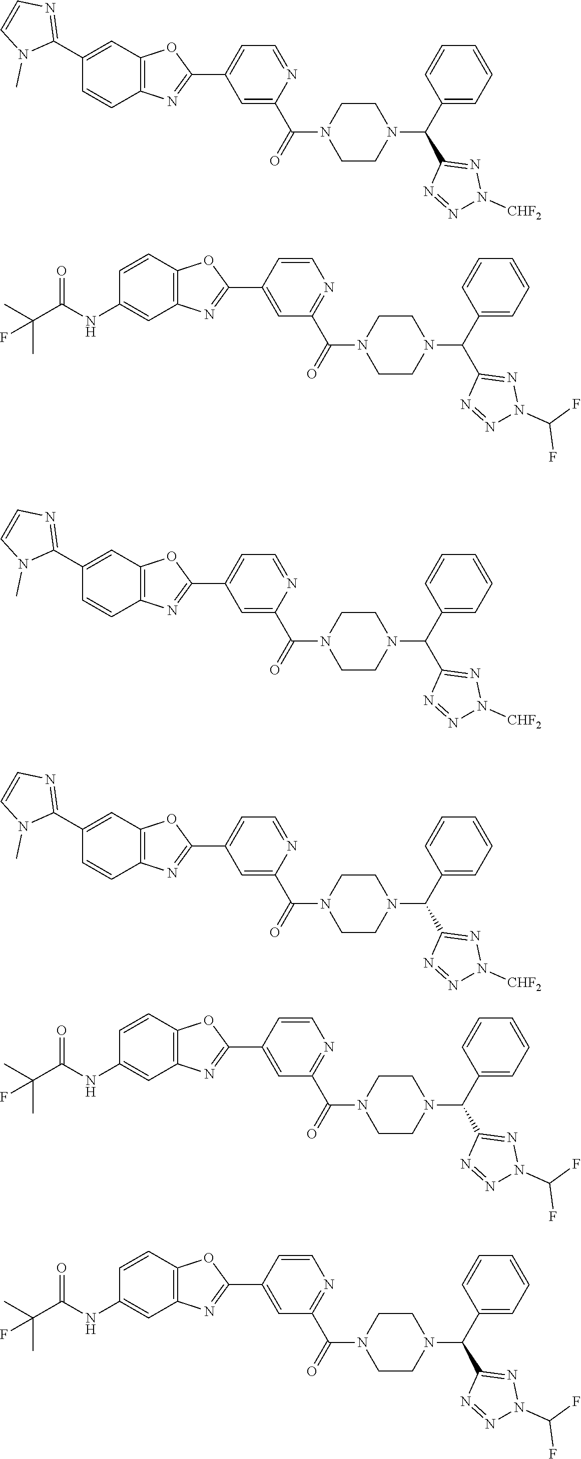

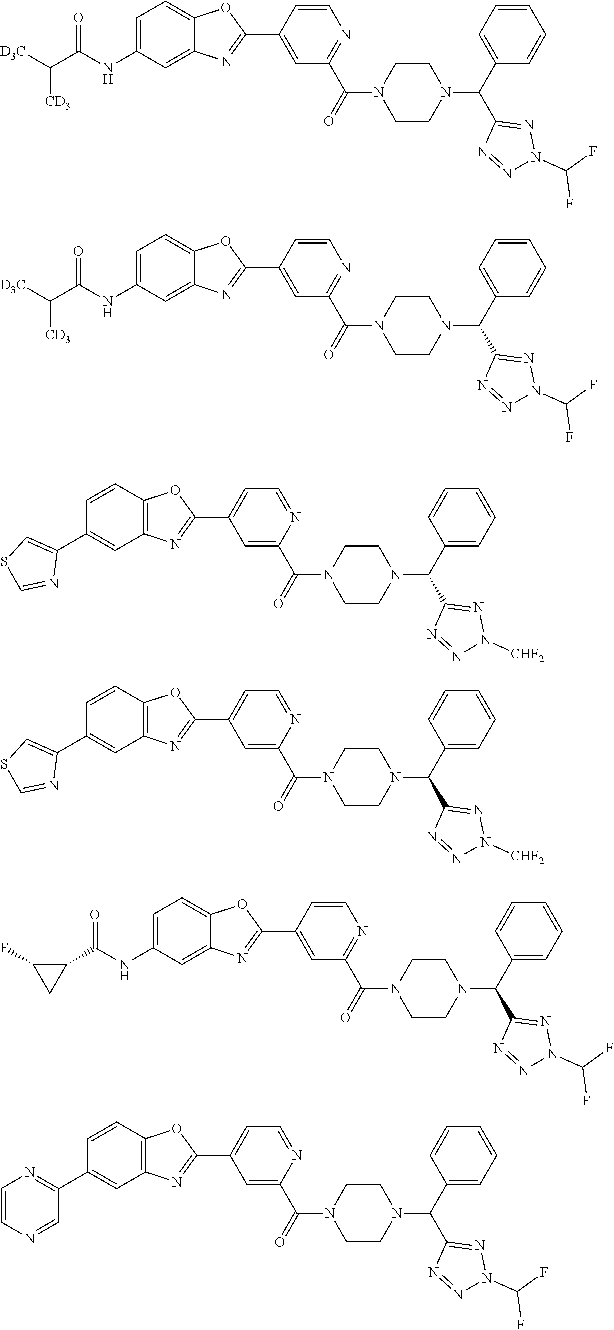

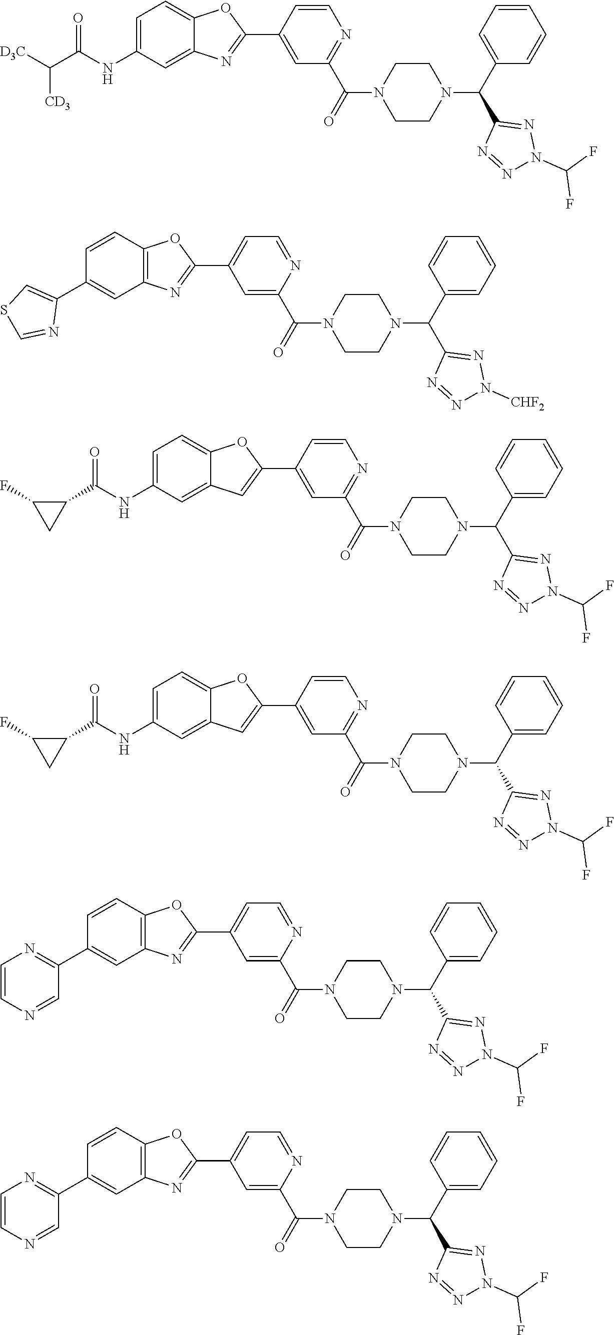

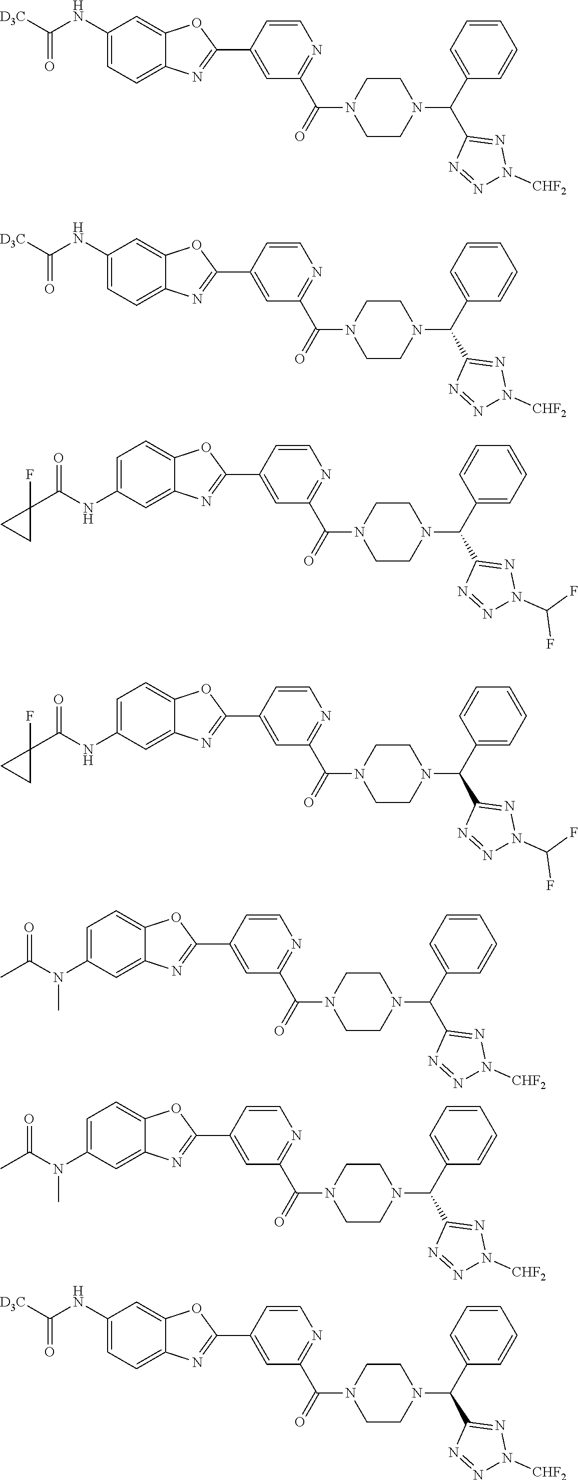

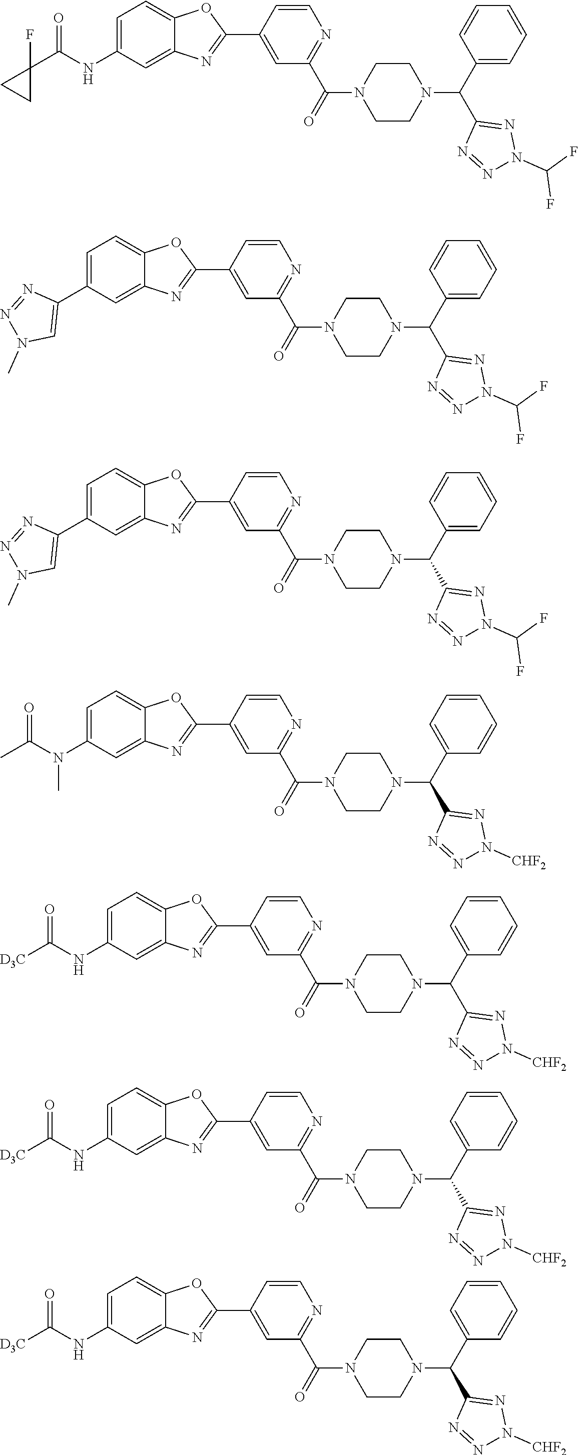

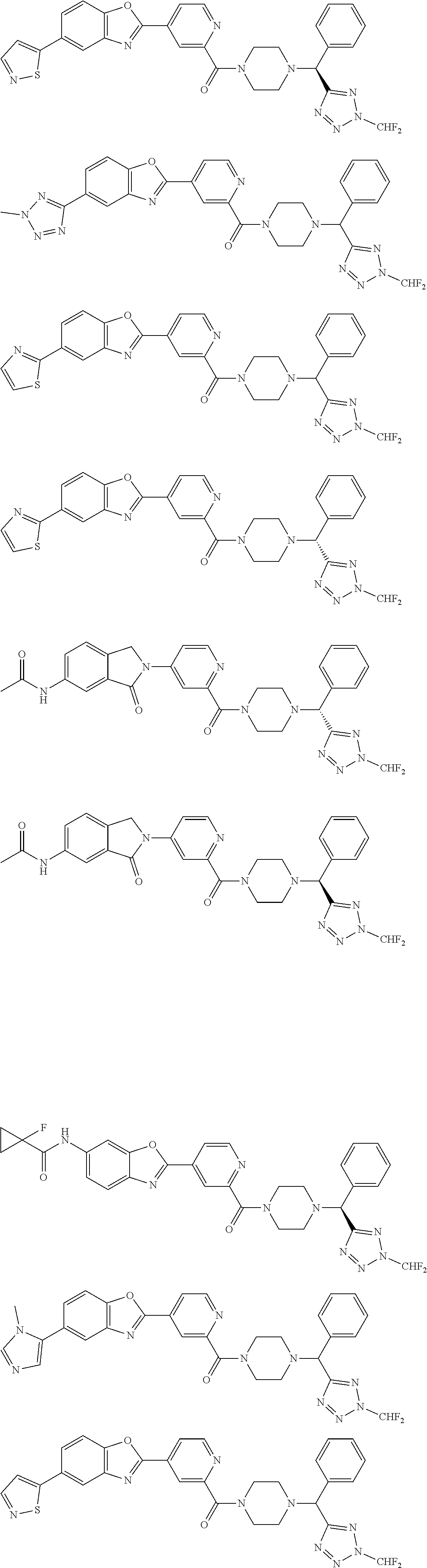

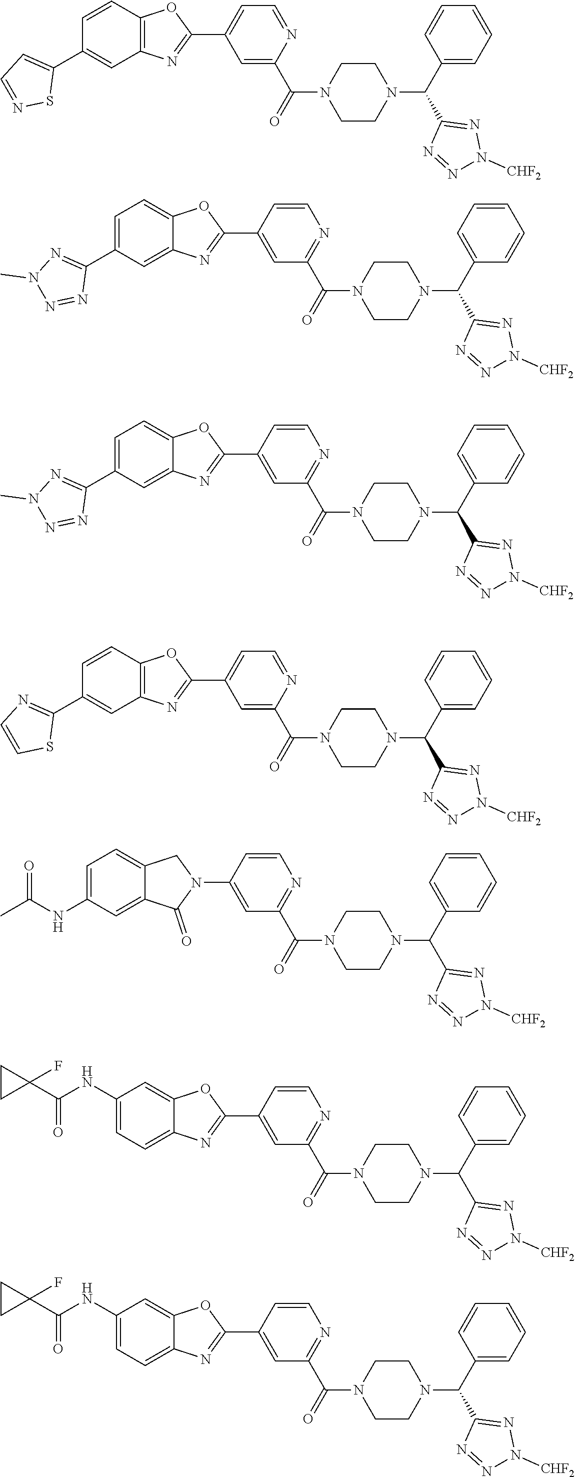

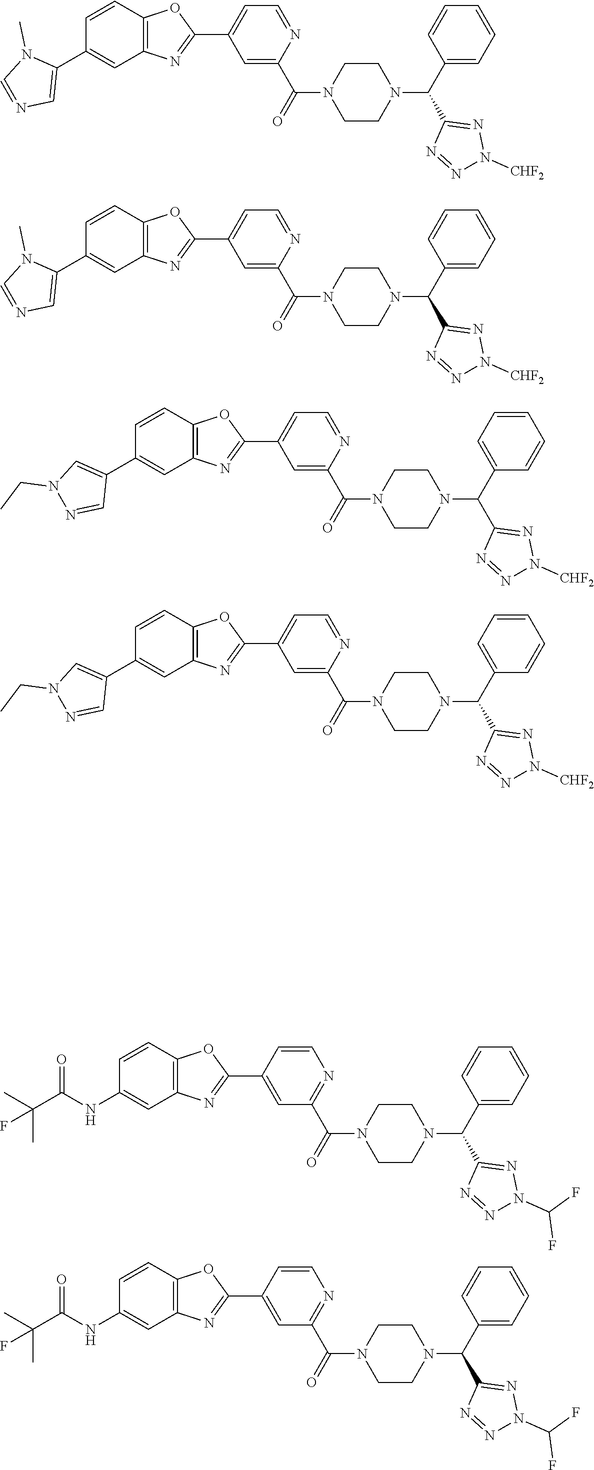

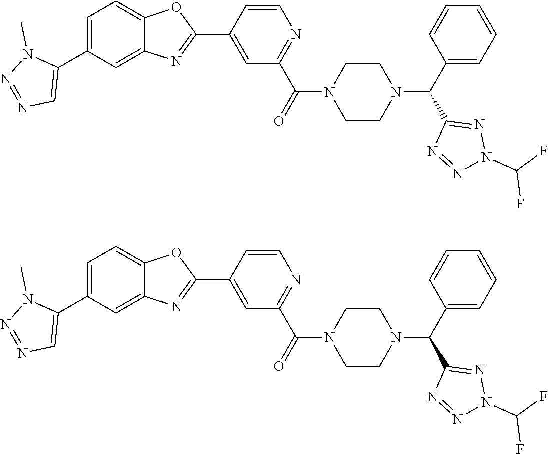 [SMILES: Cn1nncc1-c1ccc2oc(-c3ccnc(C(=O)N4CCN([C@H](c5ccccc5)c5nnn(C(F)F)n5)CC4)c3)nc2c1 ; Cn1nncc1-c1ccc2oc(-c3ccnc(C(=O)N4CCN([C@@H](c5ccccc5)c5nnn(C(F)F)n5)CC4)c3)nc2c1]

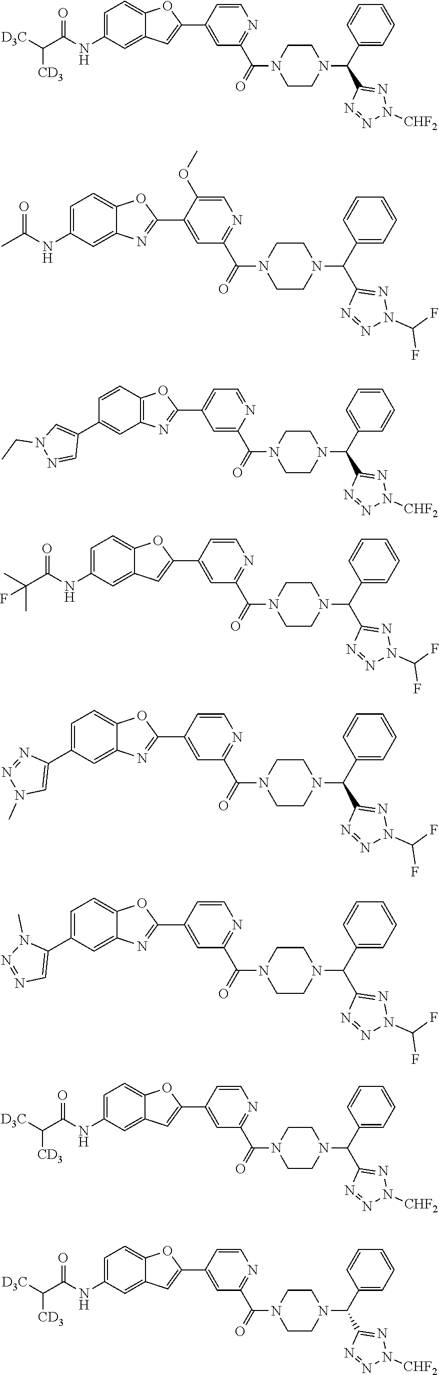

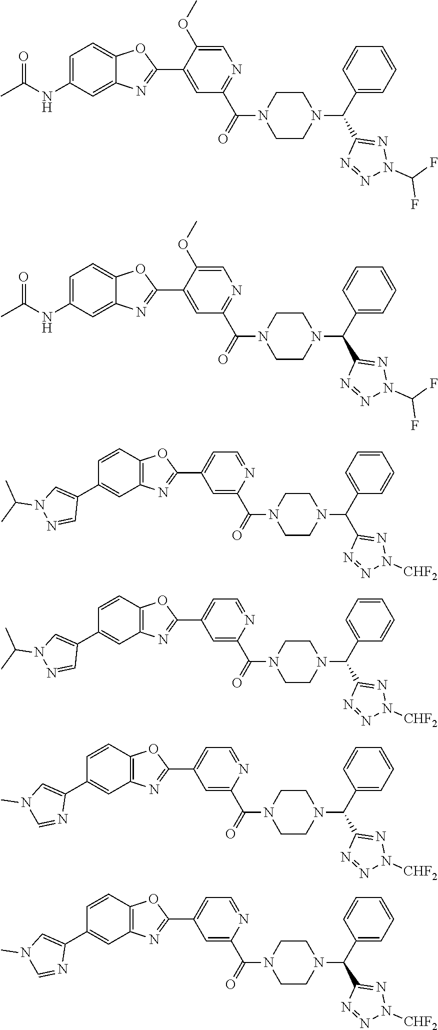

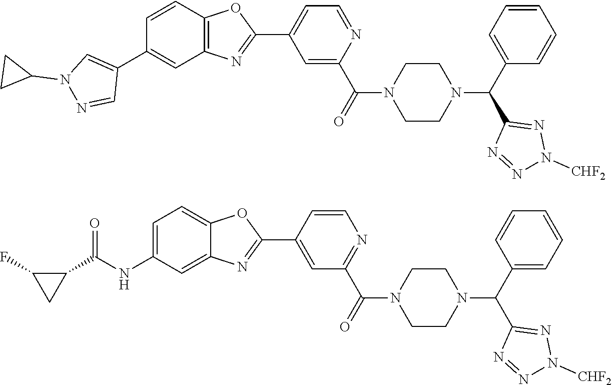

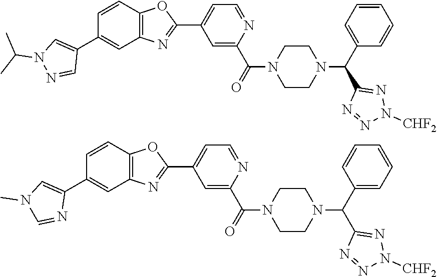

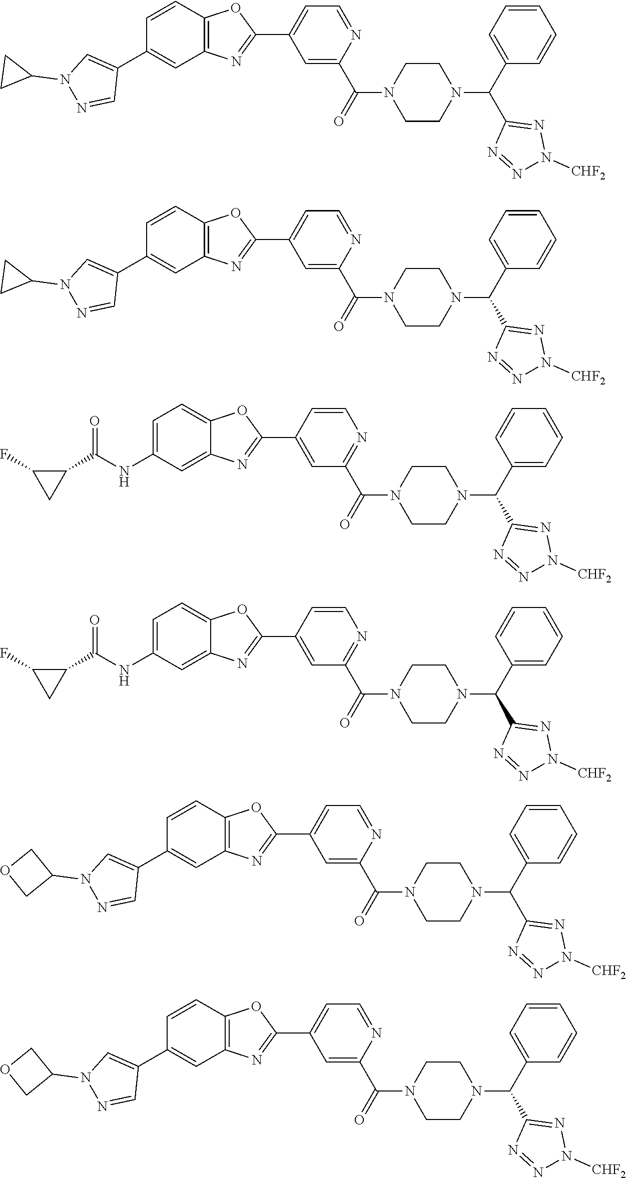

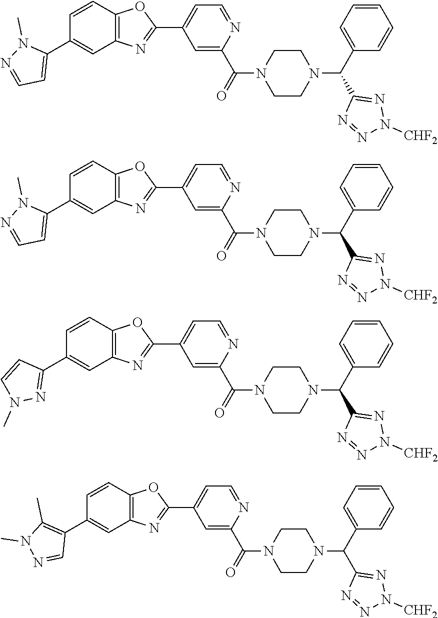

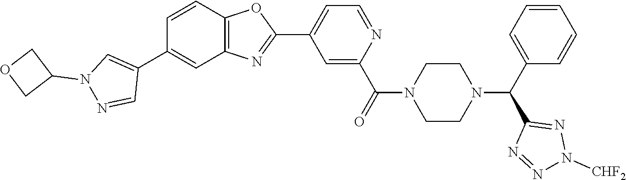 [SMILES: O=C(c1cc(-c2nc3cc(-c4cnn(C5COC5)c4)ccc3o2)ccn1)N1CCN([C@@H](c2ccccc2)c2nnn(C(F)F)n2)CC1]

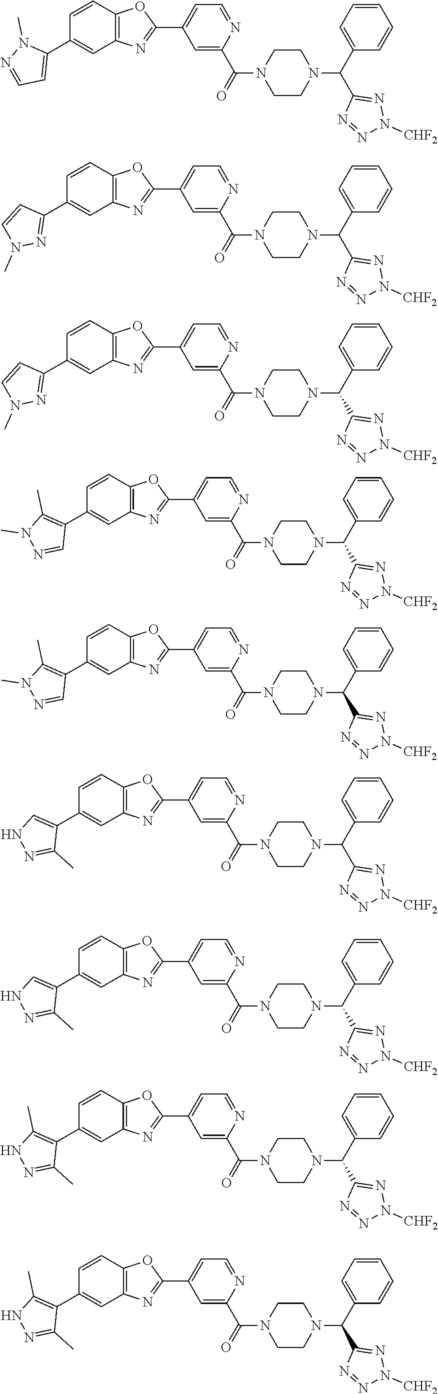

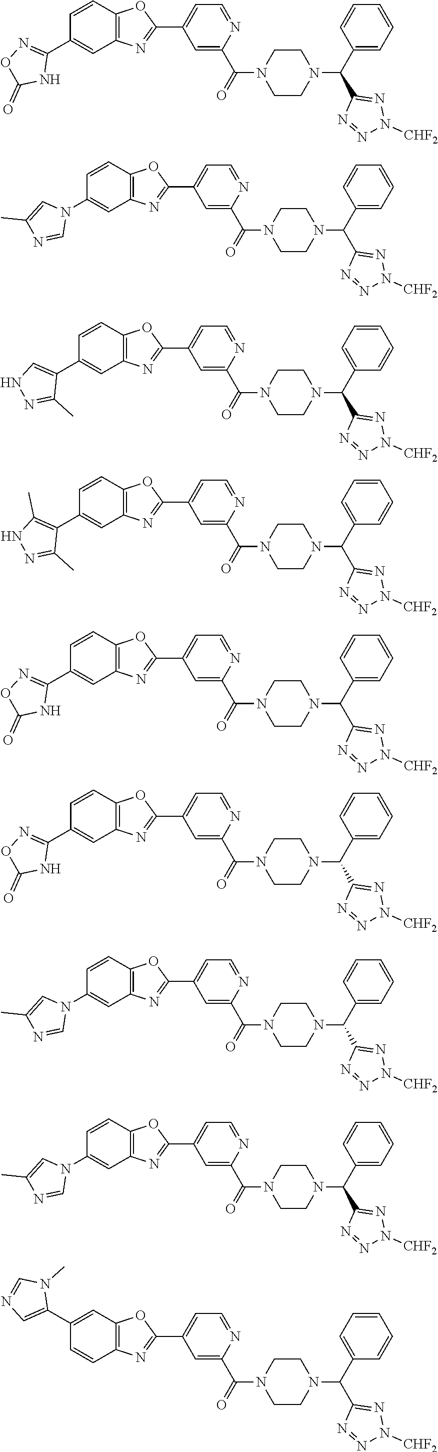

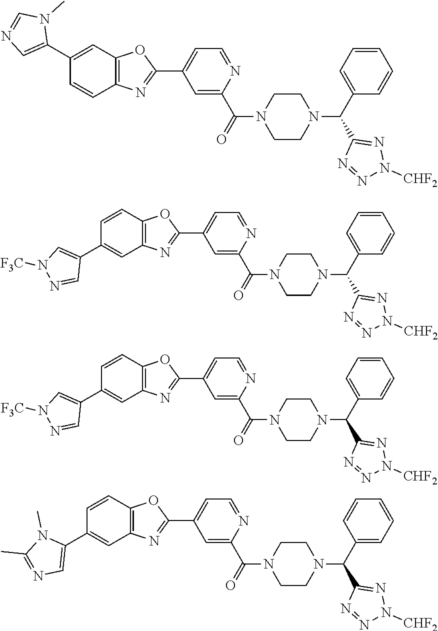

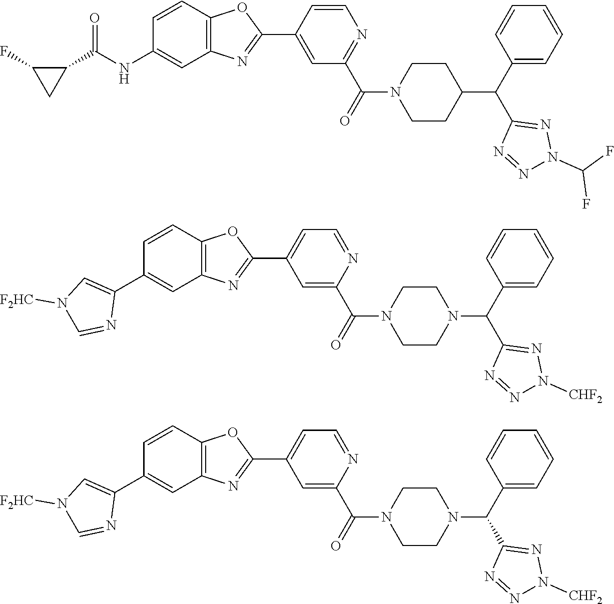

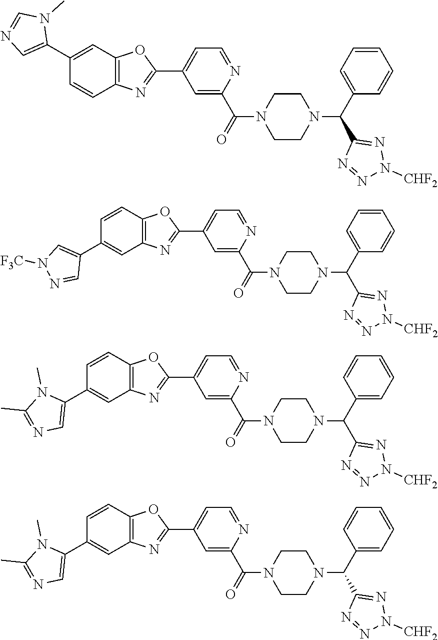

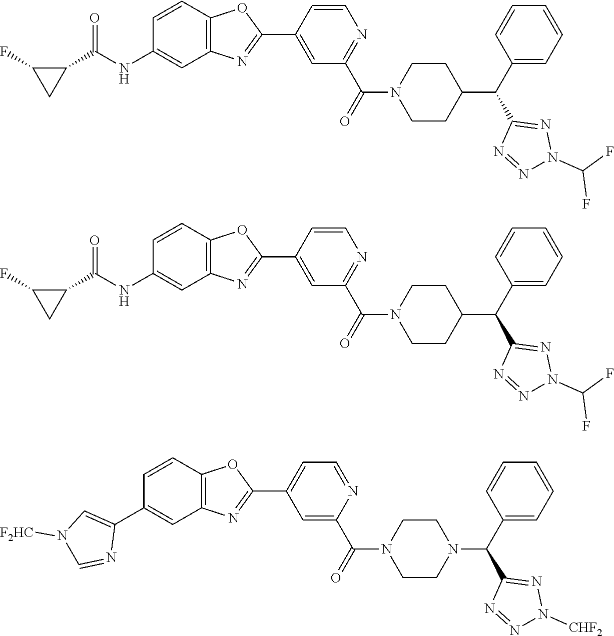 [SMILES: O=C(Nc1ccc2oc(-c3ccnc(C(=O)N4CCC([C@H](c5ccccc5)c5nnn(C(F)F)n5)CC4)c3)nc2c1)[C@@H]1C[C@@H]1F ; O=C(Nc1ccc2oc(-c3ccnc(C(=O)N4CCC([C@@H](c5ccccc5)c5nnn(C(F)F)n5)CC4)c3)nc2c1)[C@@H]1C[C@@H]1F ; O=C(c1cc(-c2nc3cc(-c4cn(C(F)F)cn4)ccc3o2)ccn1)N1CCN([C@@H](c2ccccc2)c2nnn(C(F)F)n2)CC1]

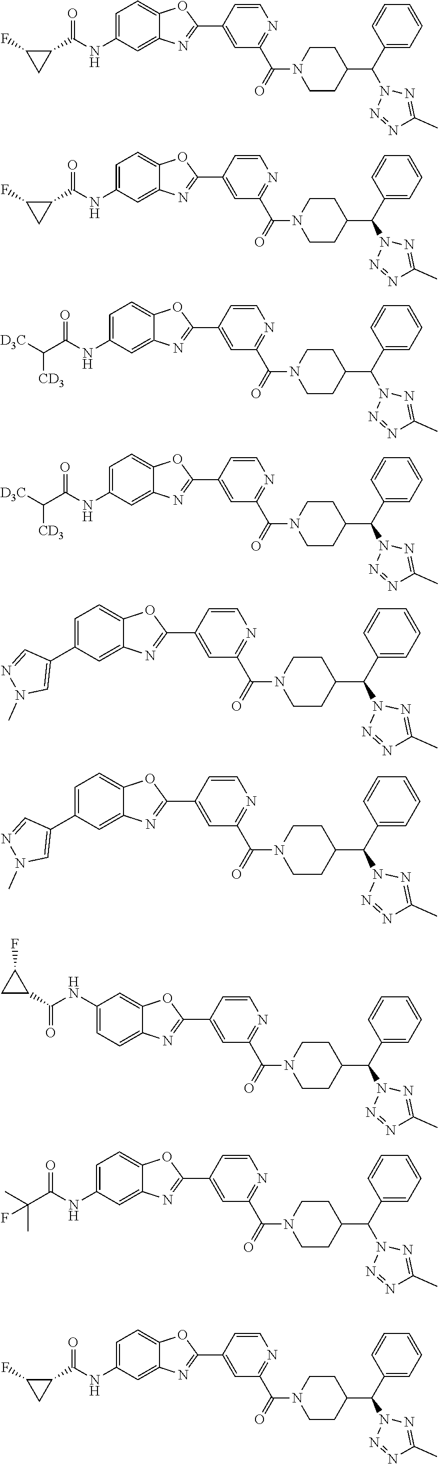

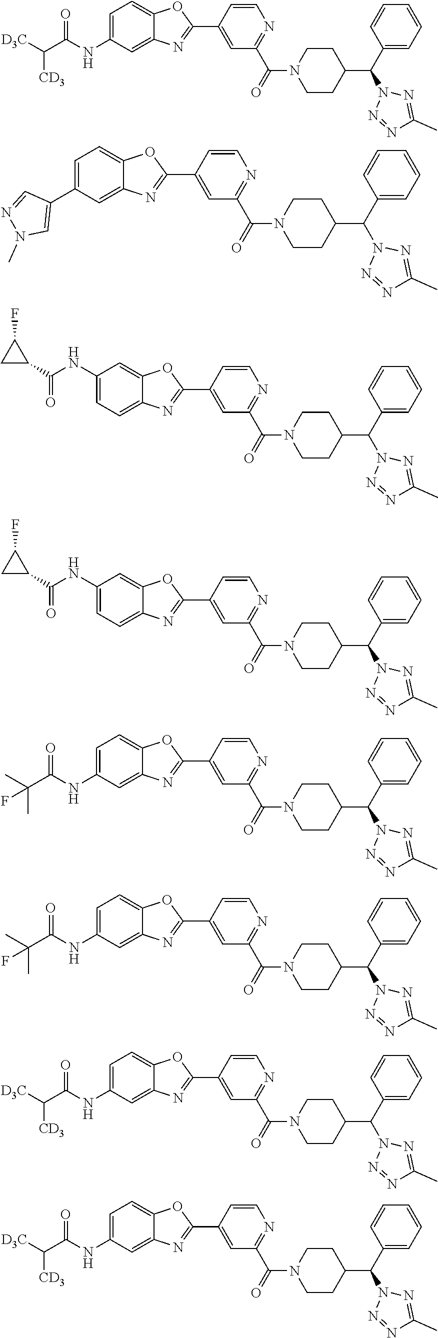

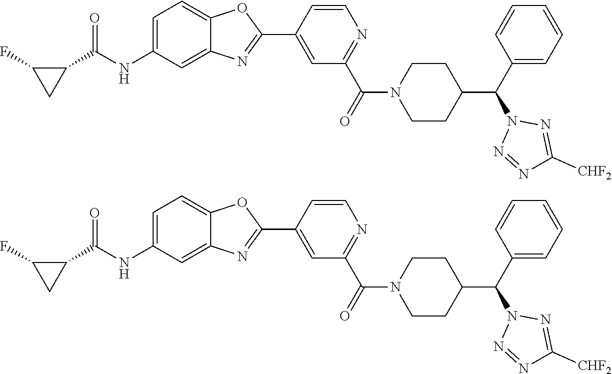

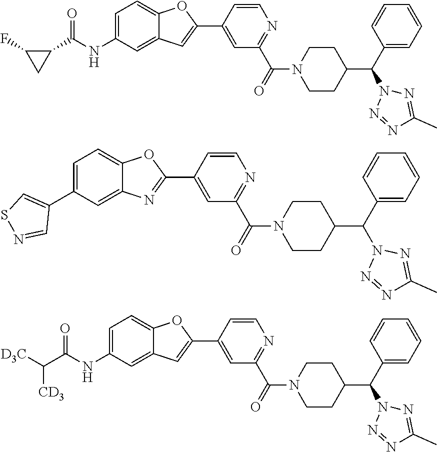

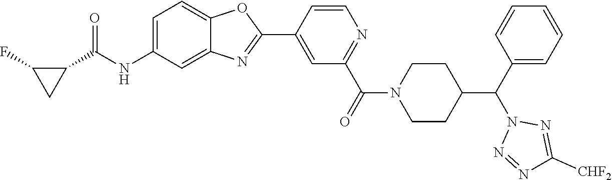 [SMILES: O=C(Nc1ccc2oc(-c3ccnc(C(=O)N4CCC(C(c5ccccc5)n5nnc(C(F)F)n5)CC4)c3)nc2c1)[C@@H]1C[C@@H]1F]

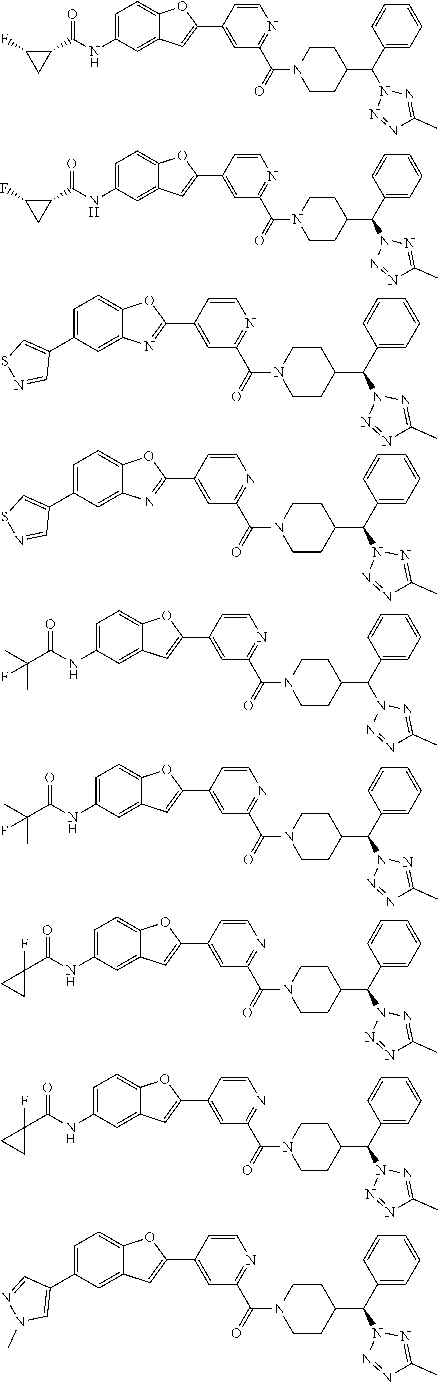

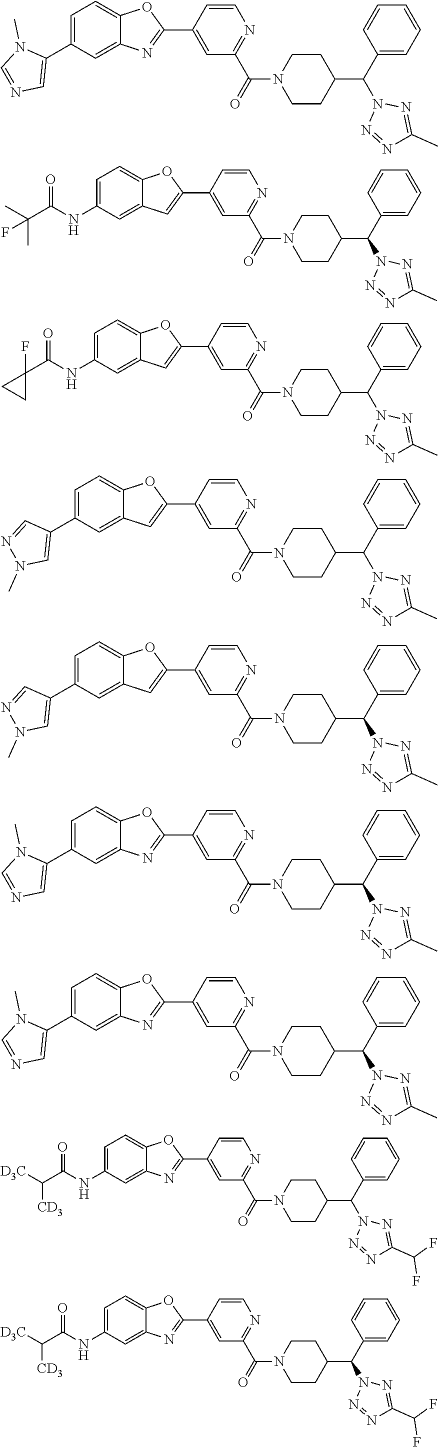

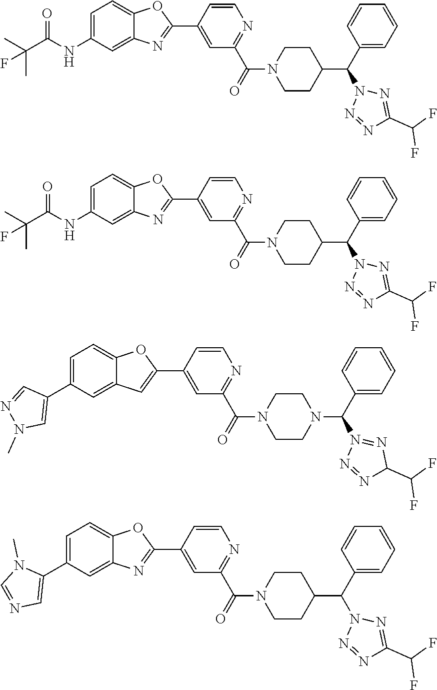

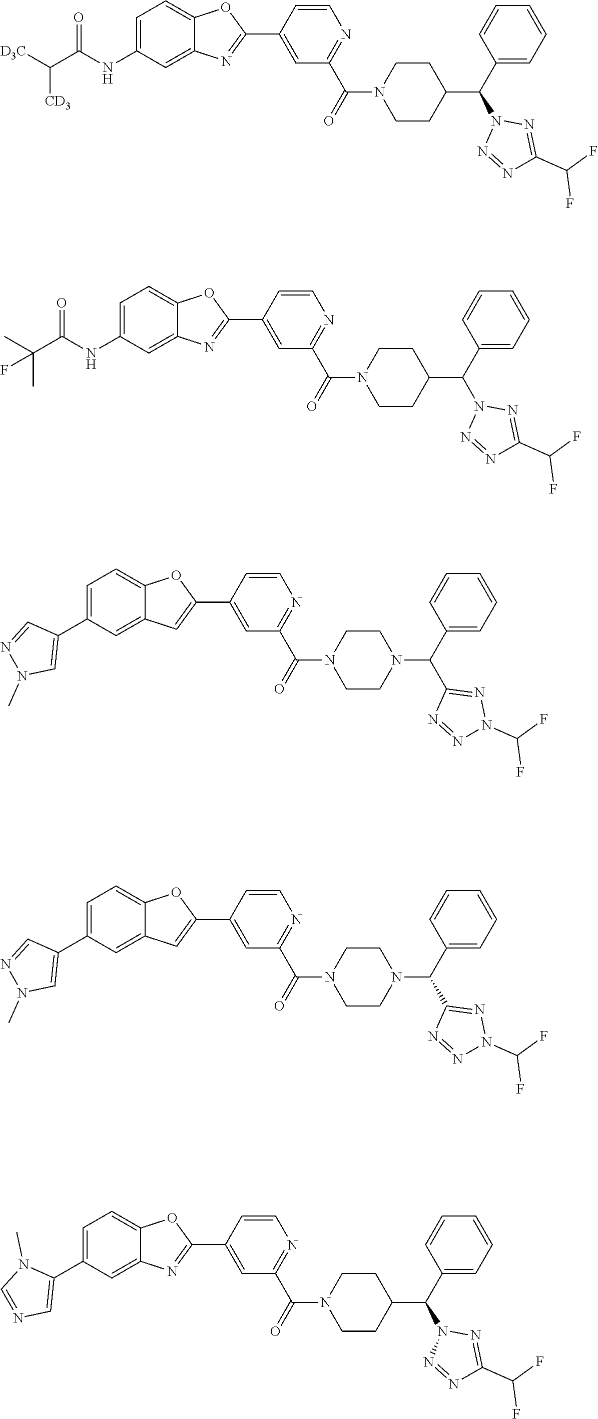

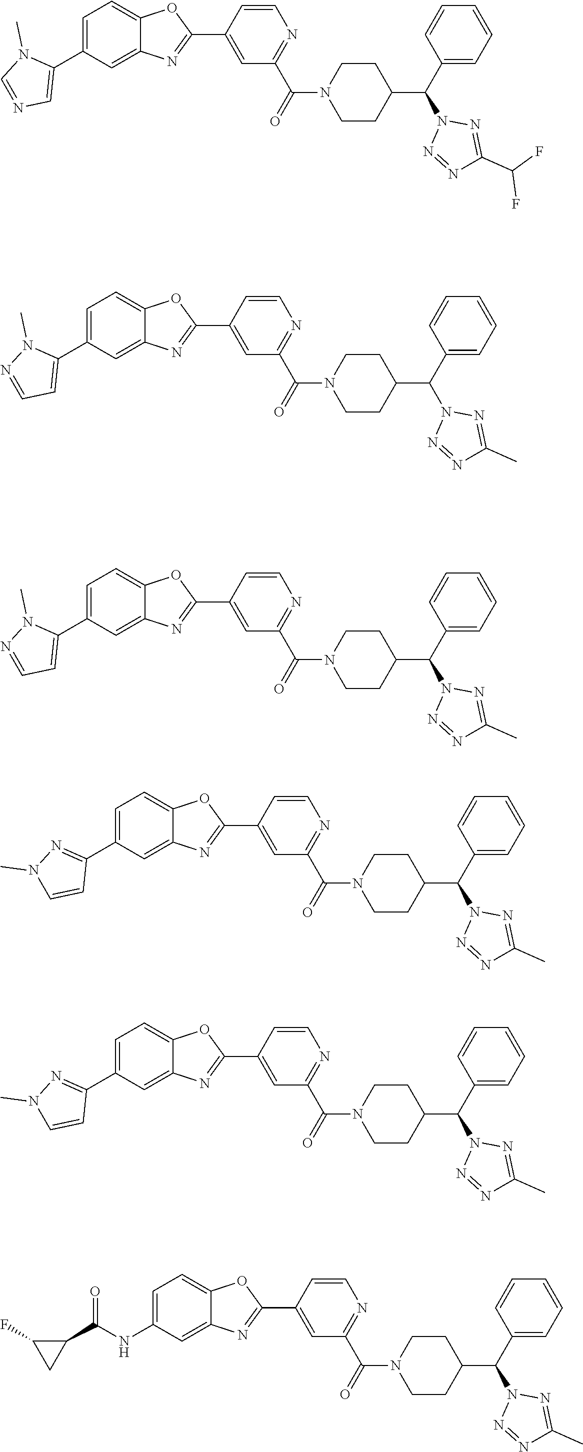

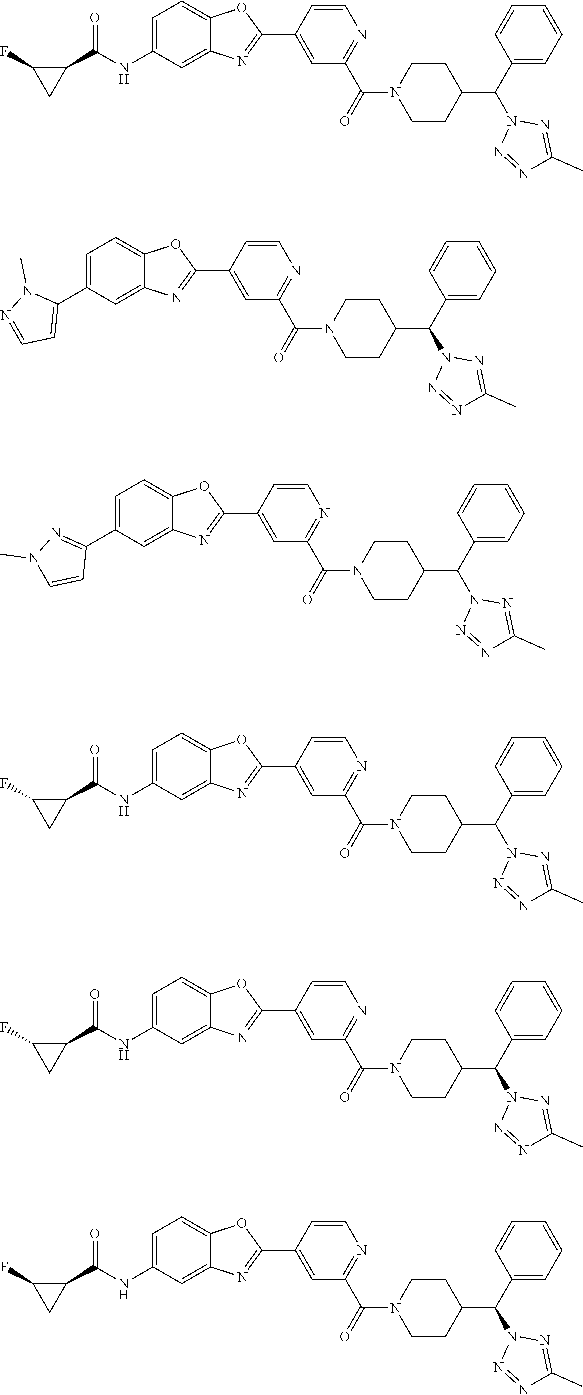

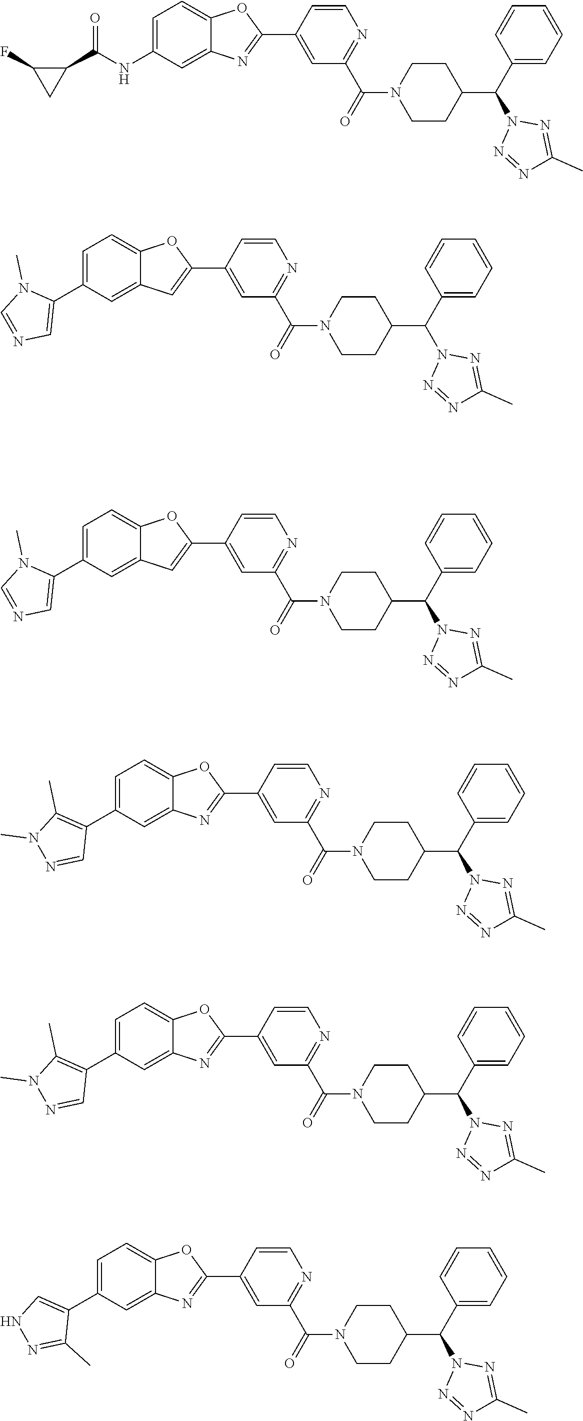

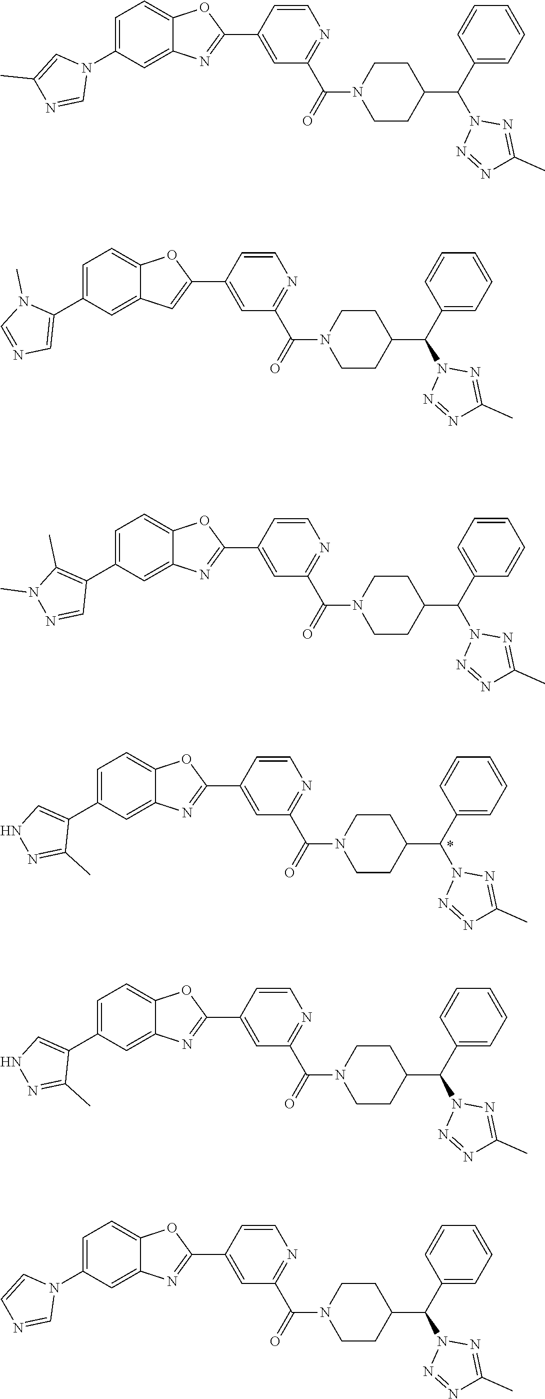

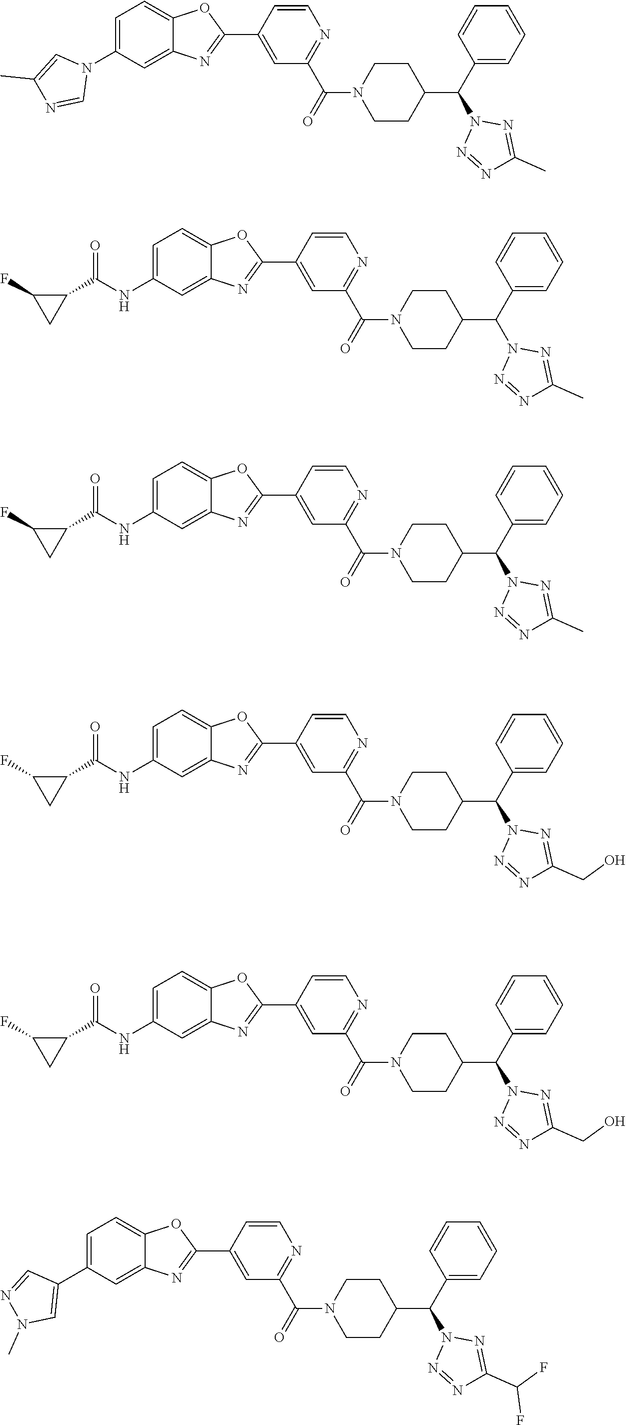

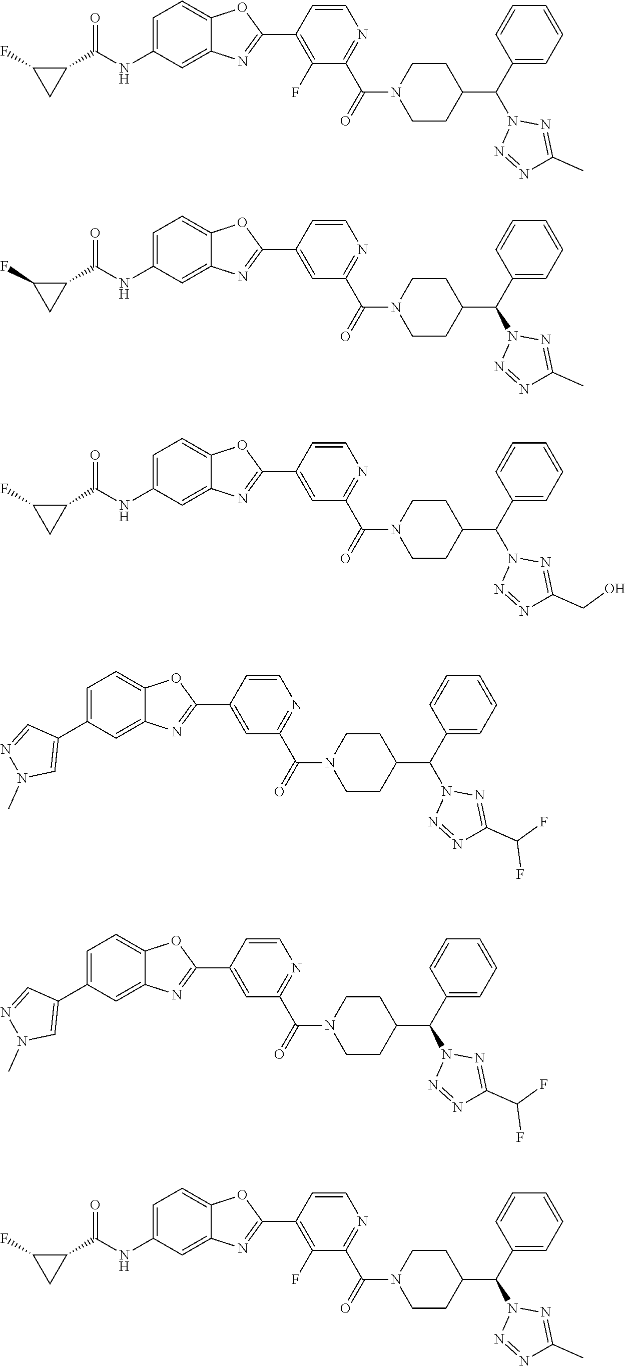

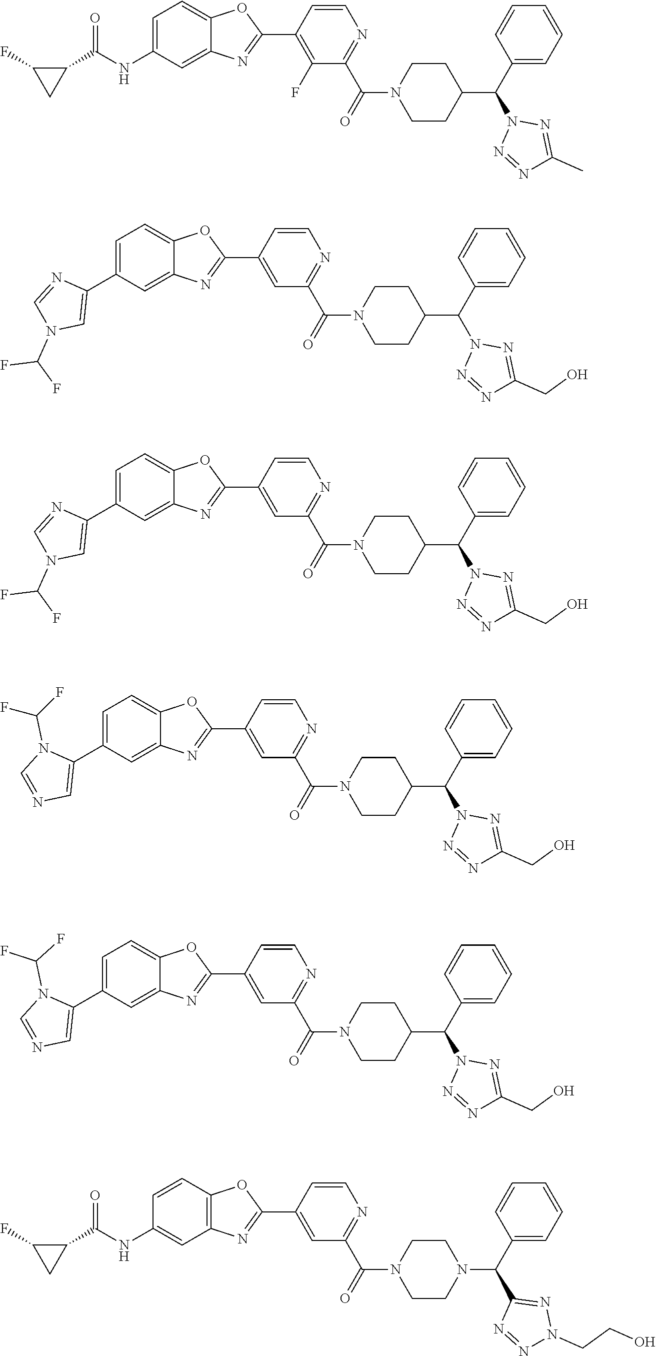

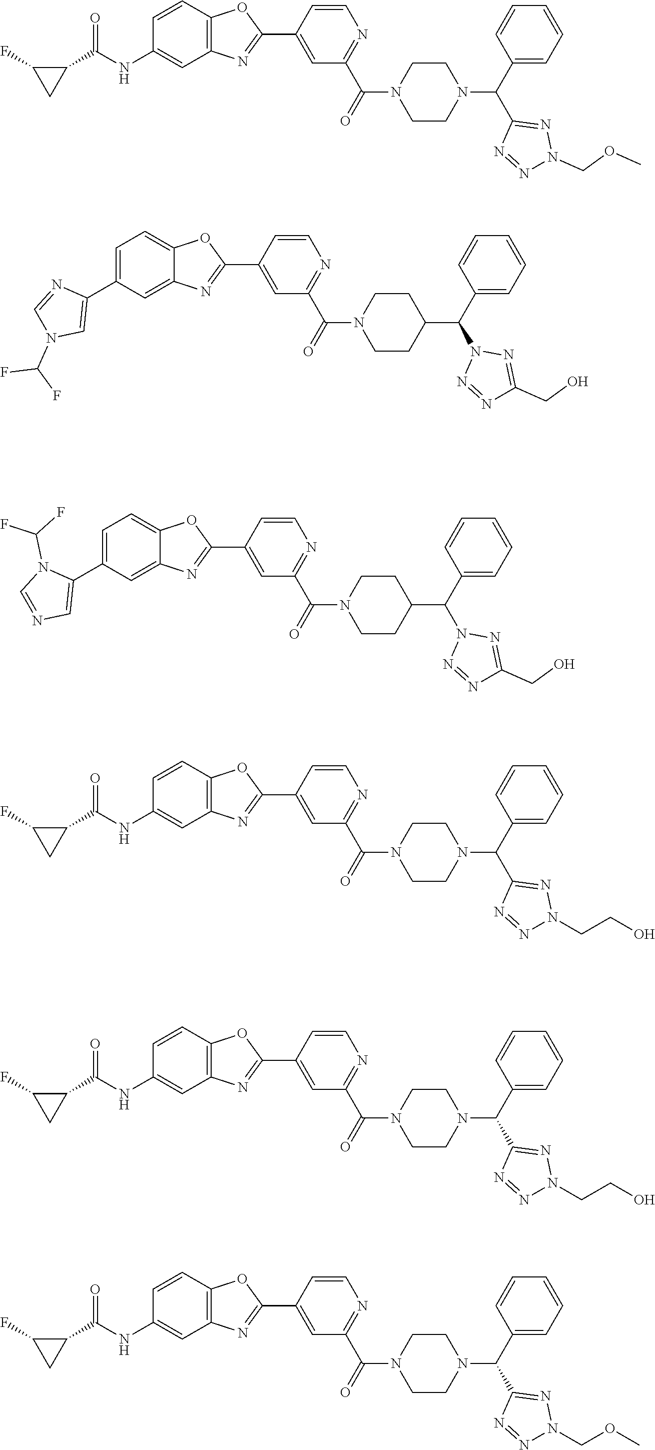

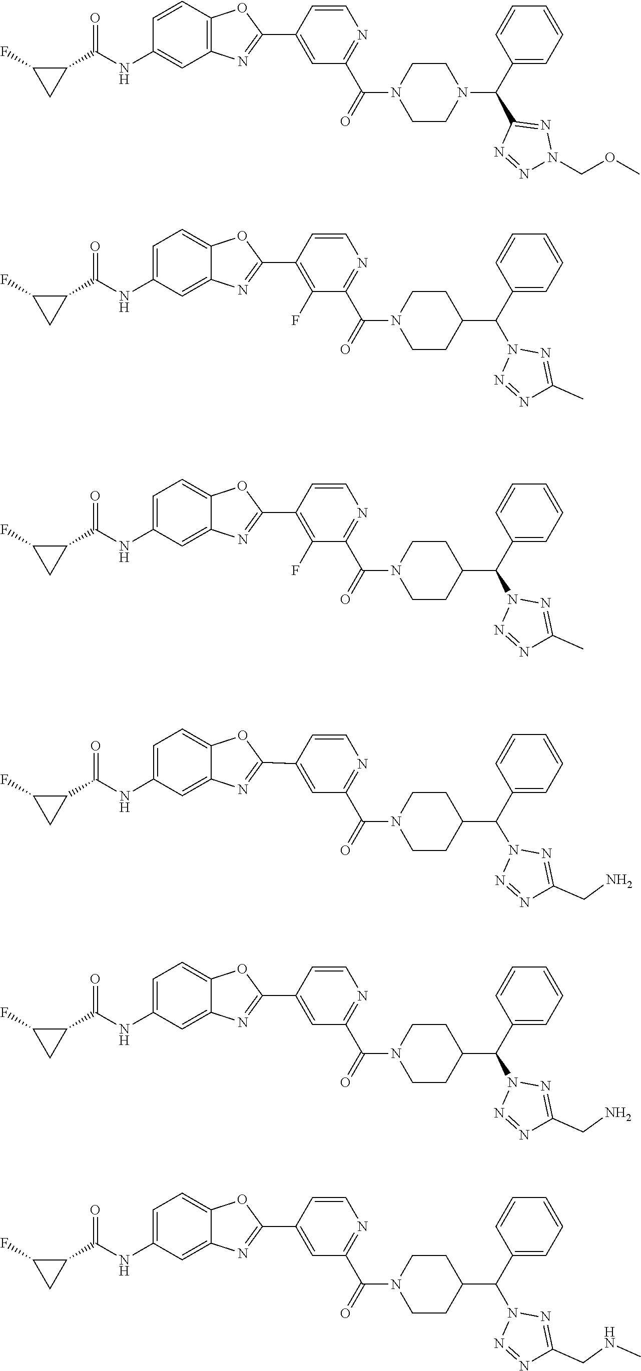

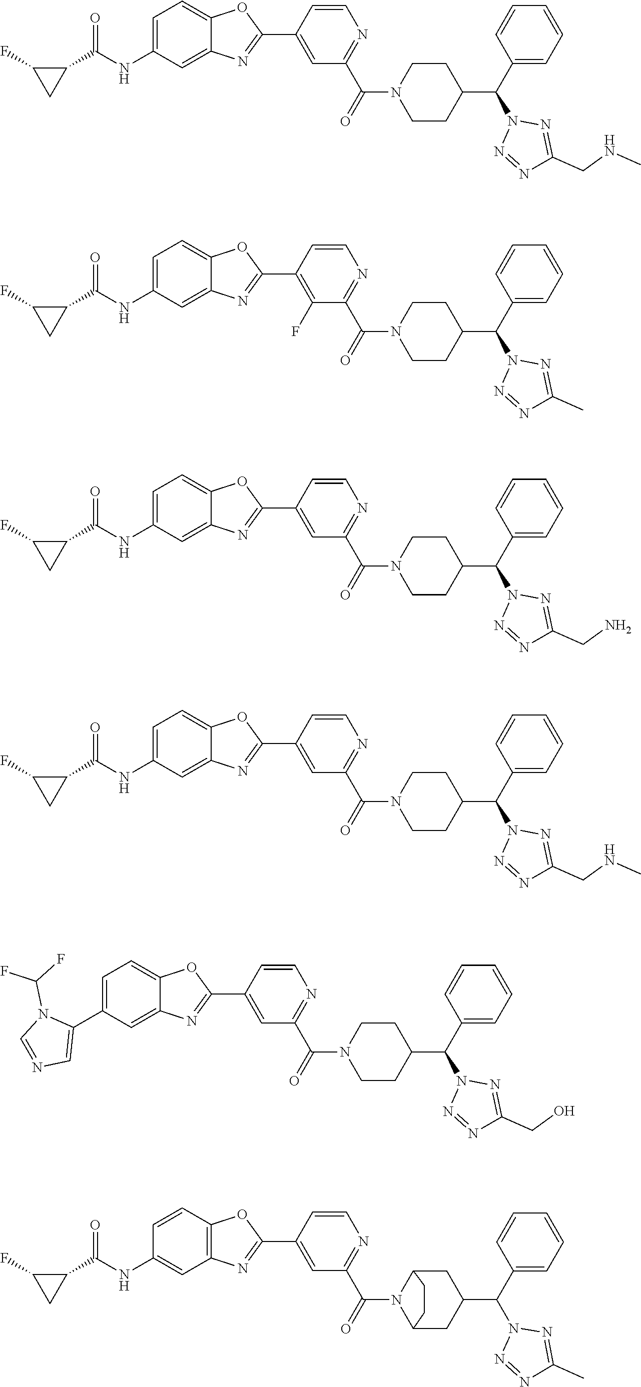

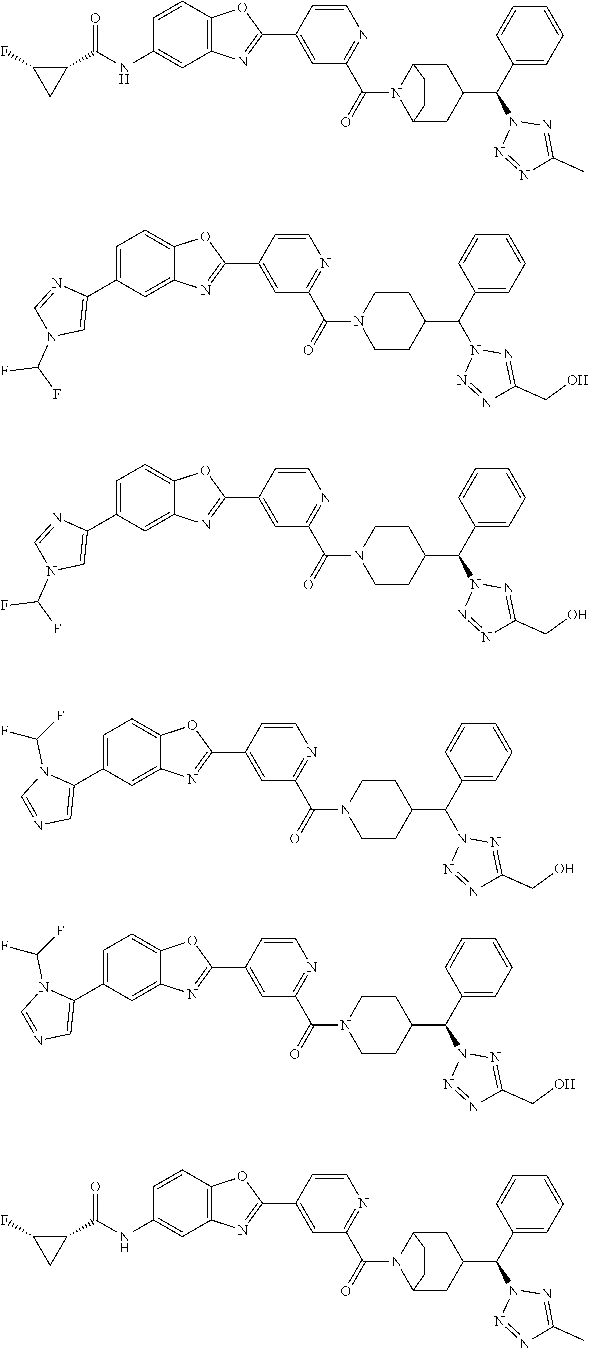

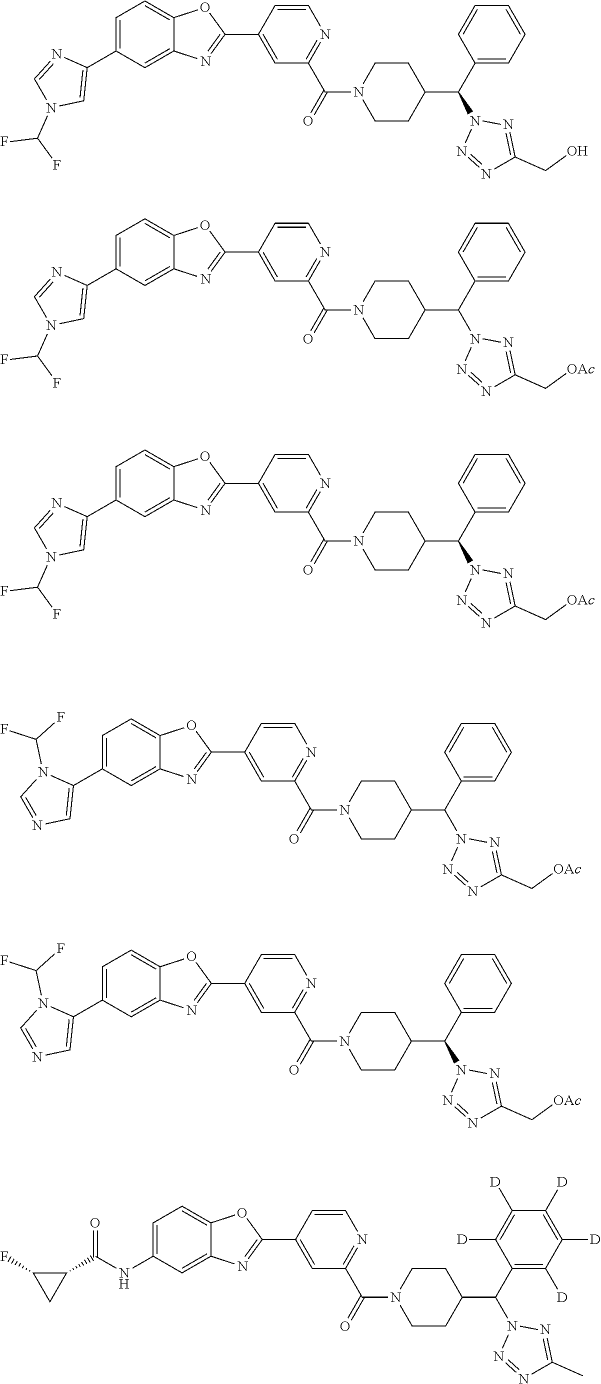

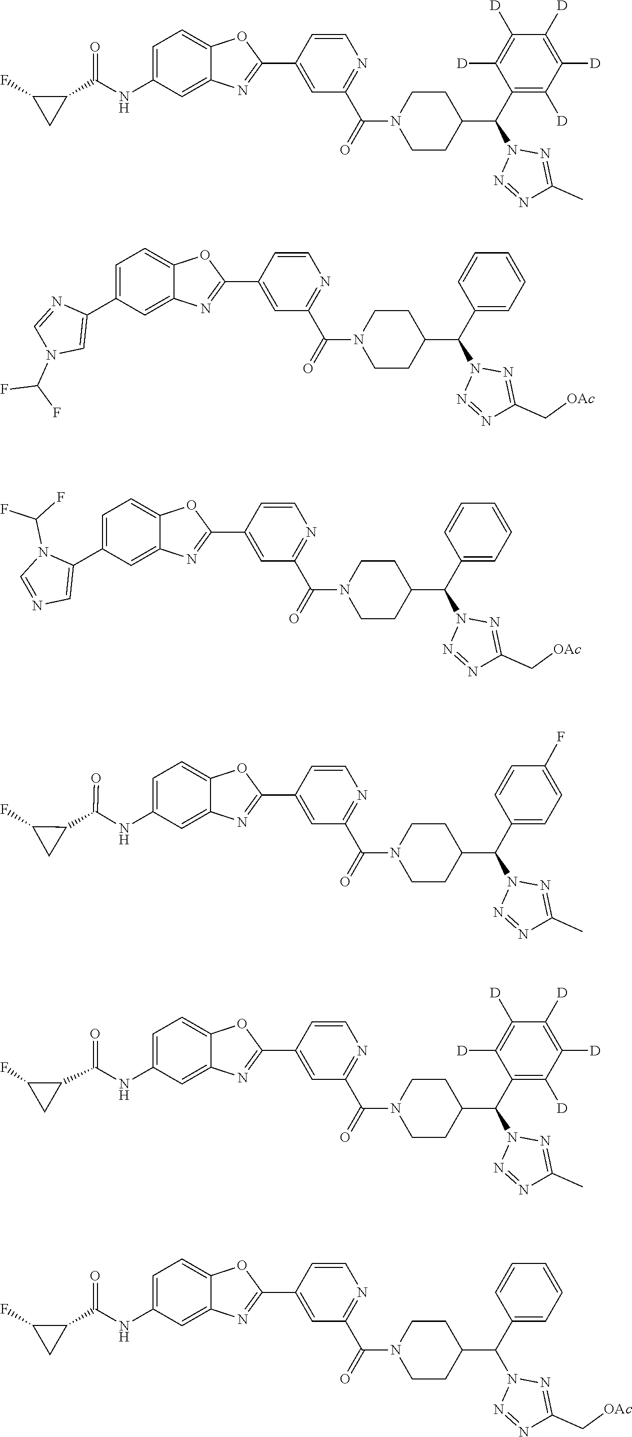

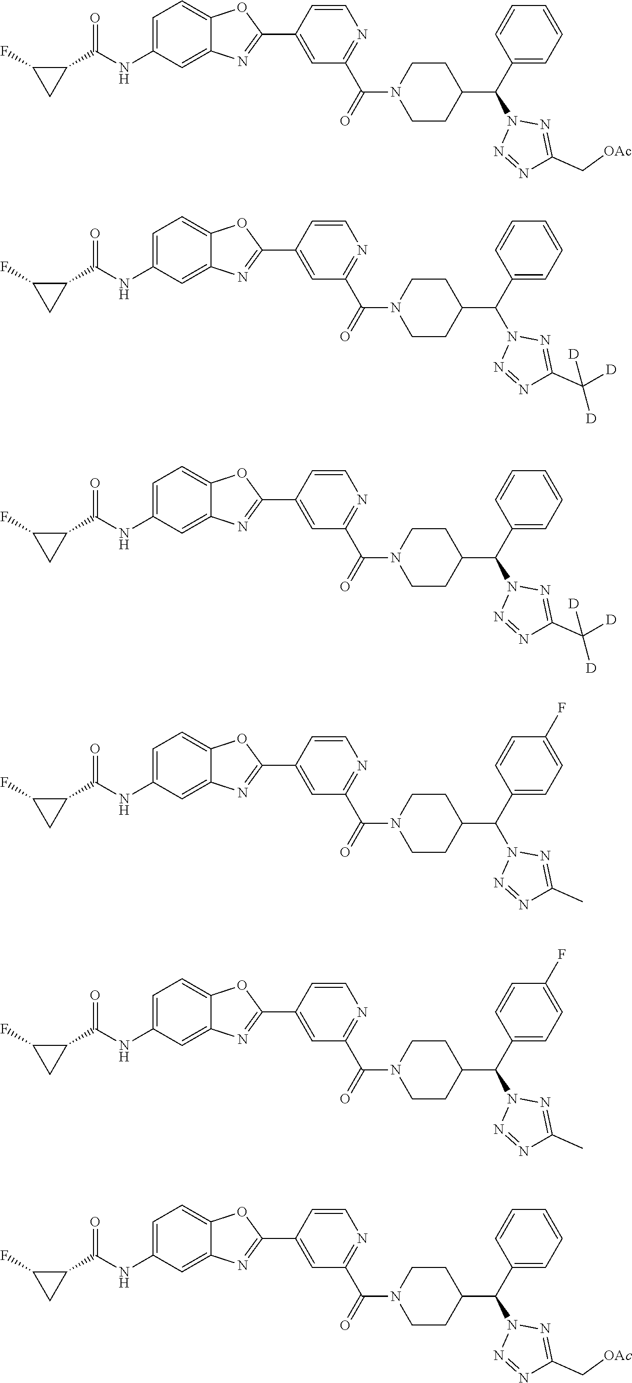

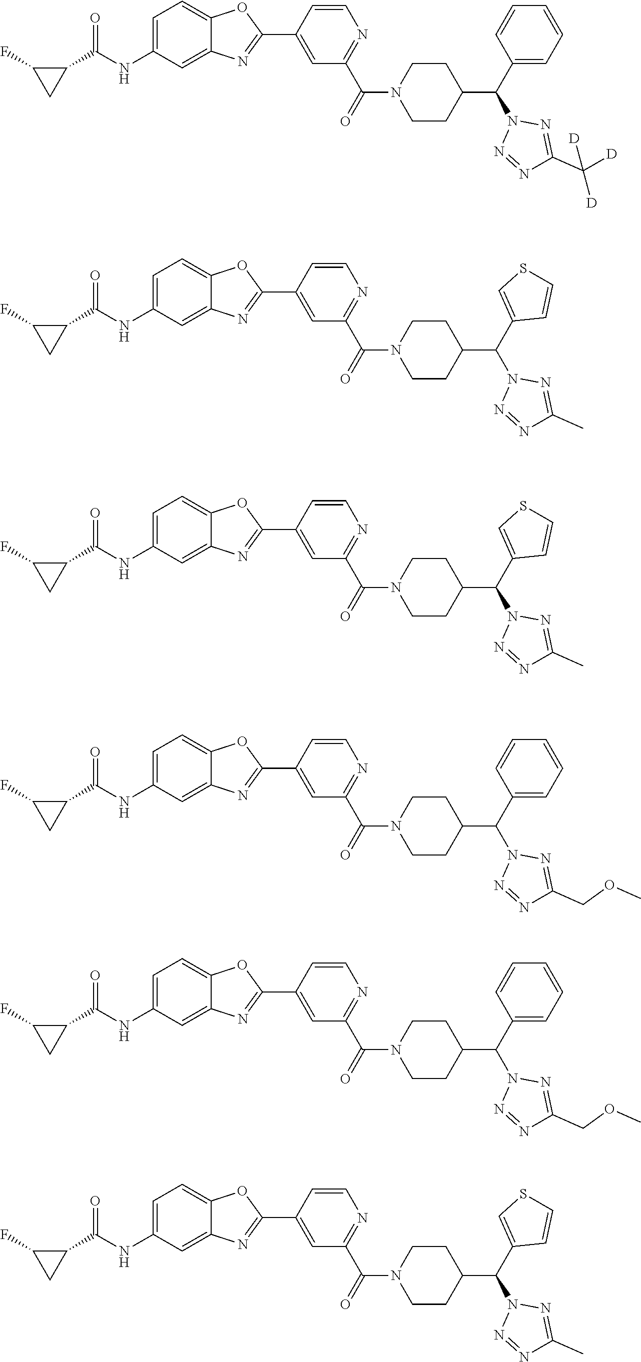

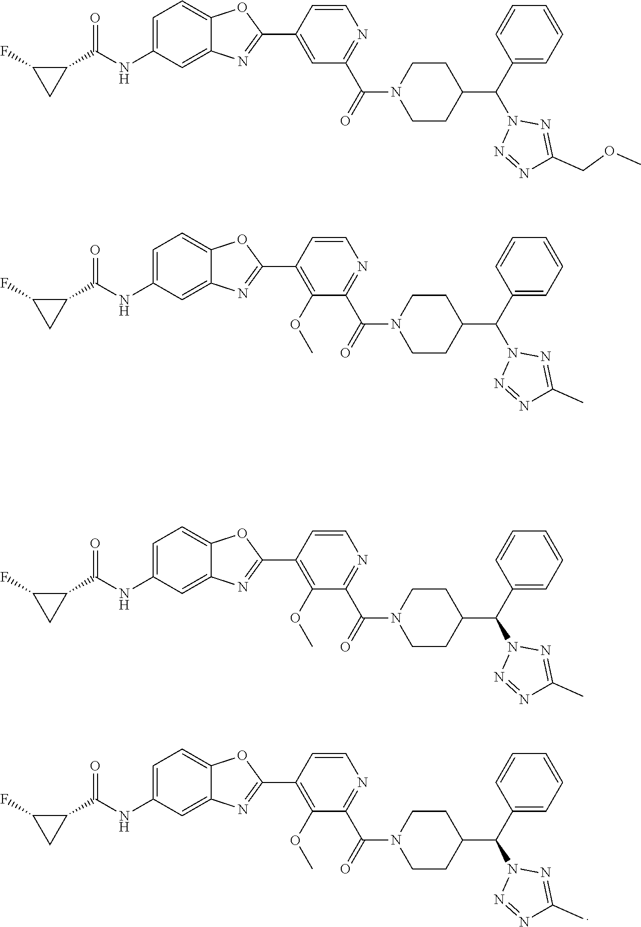

29. A pharmaceutical composition, wherein the pharmaceutical composition contains a therapeutically effective amount of the compound, the isomer thereof or the pharmaceutically acceptable salt thereof according to claim 1, and a pharmaceutically acceptable carrier and excipient.

30. A method for anti-influenza virus comprising administering the compound, the isomer thereof or the pharmaceutically acceptable salt thereof according to claim 1.

31. The compound, the isomer thereof or the pharmaceutically acceptable salt thereof according to claim 4, wherein R₁₂ is selected from

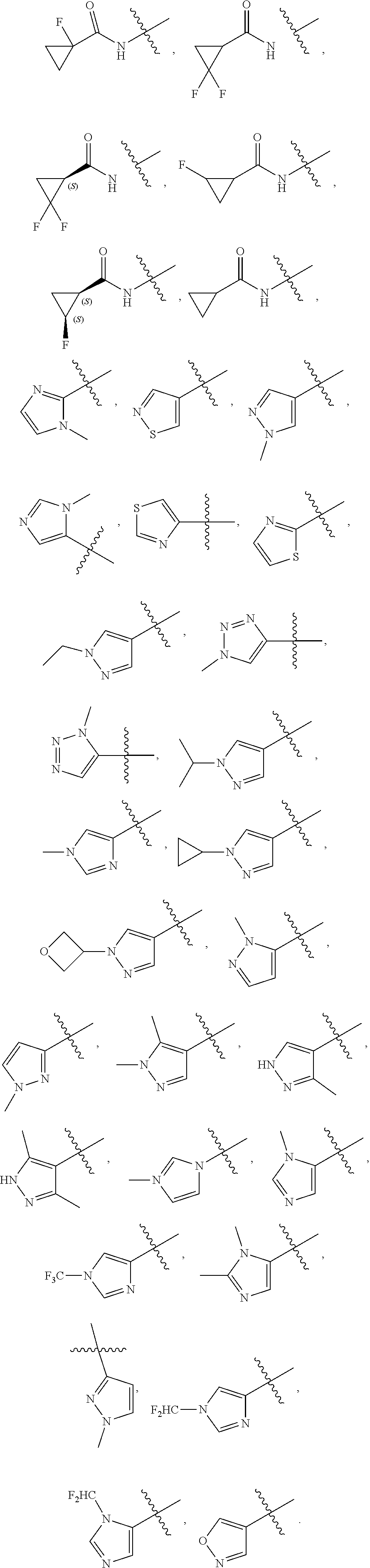

32. The compound, the isomer thereof or the pharmaceutically acceptable salt thereof according to claim 15, wherein R₁₂ is selected from

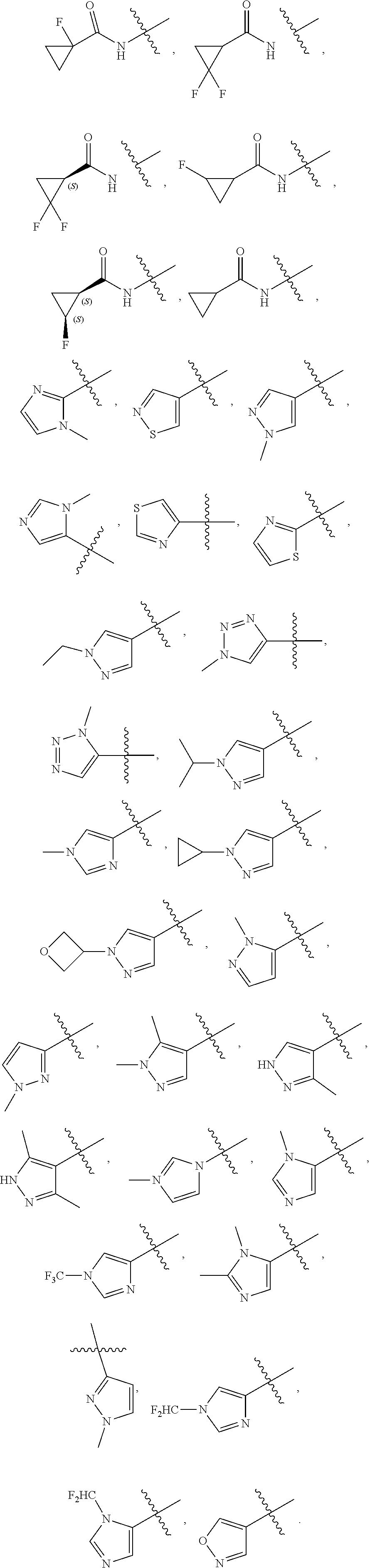

33. The compound, the isomer thereof or the pharmaceutically acceptable salt thereof according to claim 18, wherein R₁₂ is selected from [chemical structure]
